(19) United States
(12) Patent Application Publication
(10) Pub. No.: US 2012/0009151 A1
(43) Pub. Date: Jan. 12, 2012

(54) Triazines And Related Compounds Having Antiviral Activity, Compositions And Methods Thereof

(75) Inventors: Amy Qi Han, Hockessin, DE (US); Glen A. Coburn, Danbury, CT (US); Kathleen P. Provoncha, Bethpage, NY (US); Yakov Rotshteyn, Monroe, NY (US)

(73) Assignee: Progenics Pharmaceuticals, Inc., Tarrytown, NY (US)

(21) Appl. No.: 12/808,406

(22) PCT Filed: Dec. 19, 2008
(86) PCT No.: PCT/US2008/013964

§ 371 Date: Sep. 27, 2011

Related U.S. Application Data
(60) Provisional application No. 61/016,286, filed on Dec. 21, 2007.
(60) Provisional application No. 61/033,275, filed on Mar. 3, 2008.
(60) Provisional application No. 61/034,343, filed on Apr. 1, 2008.
(60) Provisional application No. 61/051,630, filed on May 8, 2008.
(60) Provisional application No. 61/110,415, filed on Oct. 31, 2008.

(51) Int. Cl. A61K 38/21; C07D 405/14; A61K 31/53; C07D 413/14; A61K 31/5377; C07D 417/14; A61K 31/538; C07D 403/12; C07D 409/12; C07D 251/48; C07D 251/42; C07D 401/12; C07D 401/14; A61K 31/55; C07D 471/18; C07D 495/04; C07D 403/04; C07D 401/04; A61K 39/42; A61K 31/7056; C12N 5/071; A01N 43/66; A01N 43/84; A01N 43/90; A01P 1/00; A61P 1/16; A61P 31/14; A61P 35/00; A61P 31/00; C07D 405/12

(52) U.S. Cl. 424/85.7; 544/198; 544/209; 514/245; 544/113; 514/236.2; 544/105; 514/230.5; 544/208; 544/211; 540/598; 514/217.05; 424/161.1; 514/43; 435/375; 424/149.1

(57) ABSTRACT

Disclosed herein are novel triazines and related compounds, the synthesis thereof, and compositions, including pharmaceutical compositions, comprising the novel triazines and related compounds. Such novel triazines and related compounds function to inhibit or block entry of viruses of the Flaviviridae family, including Hepatitis C virus (HCV), into cells that are susceptible to virus infection. These compounds are useful for the treatment, therapy and/or prophylaxis of viral diseases and infection, including HCV infection.

This application is a submission under 35 U.S.C. 371 of International Application No. PCT/US2008/013964, International Filing Date, 19 Dec. 2008, and claims benefit of U.S. Provisional Application Ser. Nos. 61/016,286, filed Dec. 21, 2007, 61/034,343, filed Apr. 1, 2008, 61/110,415, filed Oct. 31, 2008, 61/033,275, filed Mar. 3, 2008, and 61/051,630, filed May 8, 2008, the contents of all of which are incorporated by reference herein in their entireties.

This invention was made in part with support under United States Government Grant No. R44AI051134 from the National Institute of Allergy and Infectious Diseases, National Institutes of Health. The U.S. Government has certain rights in the invention.

### TECHNICAL FIELD OF DISCLOSURE

The present invention generally provides derivatives and sub-derivatives of nitrogen-containing heterocyclic compounds, azines, and amino- and alkoy-substituted 1,3,5-triazines—their stereoisomers, polymorphs, solvates, prodrugs, all salts thereof, particularly pharmaceutically acceptable salts, synthetic methods for their preparation, pharmaceutical compositions of the same, and methods for their therapeutic and prophylactic utilization. All such compounds may be useful in general embodiments for the treatment of viral diseases of the Flaviviridae family, and in one embodiment, for therapy for acute and chronic infections by hepatotrophic virions of the Hepatitis C class (NANB, Non-A, Non-B virus, HCV).

All references cited in this specification, and their references, are incorporated by reference herein where appropriate for teachings of additional alternative details, features, and/or technical background.

### INTRODUCTION

Hepatitis C virus (HCV), a virus of the family Flaviviridae and genus Hepacivirus, is responsible for chronically infecting approximately 170-200 million persons worldwide, roughly 3% of the current population of 6.6 billion (1). Infection predominantly occurs via the percutaneous exchange of infected blood. The initial infection fails to clear in most instances, and chronic hepatitis, resulting in decompensated liver disease or hepatocellular carcinoma occurs in many cases. Other pathologies associated with chronic HCV infection are mixed cryoglobulinemia, overt B-cell non-Hodgkin's lymphoma, and idiopathic pulmonary fibrosis (2).

HCV is structurally related to hepatitis G (HGV-C), GBV-A and GBV-B viruses that infect Tamarin monkeys, West-Nile virus, dengue fever, and yellow fever viruses (3). HCV shows considerable intra-genomic diversity, existing in at least 6 major genotypes, with at least 50 subtypes having been identified.

The US Center for Disease Control estimates that 1.8% of United States inhabitants show seropositivity for HCV antibodies. Roughly 3 out of 4 of these seropositive individuals are also viremic, presenting acute or chronic active infections. HCV infection accounts for roughly 30,000 new, acute infections and 8,000 to 10,000 deaths yearly in the United States.

The continued failure to develop a highly efficacious treatment for chronic HCV infection is well known, as are the difficulties and experimental uncertainties in developing efficacious medicaments (4). The most effective, proven therapeutic regimen for HCV infection is a combination therapy incorporating alpha-interferon (IFN-α) or pegylated IFN-α and ribavirin, 1-(β-D-Ribofuranosyl)-1H-1,2,4-triazole-3-carboxamide. This regimen is substantially more efficacious against infections of HCV genotypes 2 and 3, compared to genotype 1, as measured by sustained viral response. Genotype 1, comprised of subtypes 1a and 1b, is the major infective agent in the United States, constituting roughly 80% of reported cases (4). The detailed mechanism of ribavirin interaction with the viral life-cycle is not well defined, but IFN-α probably functions as a general inhibitor of viral replication as well as favorably modulating the host's antiviral immune response (4).

HCV is an enveloped, positive sense RNA virus possessing a −9.6 kb genome with a single open reading frame. The virus is approximately spherical in shape with a diameter of about 60 nm (FIG. 1). In the intact virus the genome resides in an icosahedral core. The genome is translated into a single ˜3,000 amino acid polyprotein directed from an internal ribosome entry site (IRES) located within the 5′ non-translated region. The structural proteins are released from the polyprotein by cellular peptidases, whereas non-structural proteins are cleaved by virally encoded proteases.

Two of the structural subunits, the envelope glycoproteins, E1 and E2, form heterodimers and mediate the process of viral attachment, fusion and entry (5,6). The envelope protein E2 possesses a binding site for CD81, a tetraspannin receptor expressed on the cell surface of hepatocytes that acts as a receptor or co-receptor of the HCV viral particle (6).

CD81 is necessary but not sufficient for HCV entry. The expression of CD81 alone cannot explain the cellular tropism exhibited by HCV, because this receptor is ubiquitously expressed by a large number of tissue types (6). VanCampernolle et al. (5) proposed that cellular permissivity relates to sympathetic mutations in a helical segment of the second extracellular loop of CD81, known as helix D (5). Therein, residues I(182), N(184), and F(186) have been shown to be particularly important for CD81 binding to HCV-E2 (11), and as such, are targets for competitive inhibition and pharmaceutical intervention.

Fusion of the viral capsule with the lipid membrane of a potential host cell is important for viral entry into the cell and is thought to occur by a low-pH endocytotic process mediated by CD81, as shown in FIG. 2 (7).

HCV research has been hampered by the lack of suitable infectivity models, but recent advances have demonstrated that unmodified HCV envelope proteins can pseudotype retroviral particles and thereby mediate cell entry. Details of HCV tropism and cell entry can now be studied, because such HCV pseudovirus particles (HCVpp) seem to accurately replicate early stages of the viral life cycle (6-10). Such HCVpp accurately reproduce the essential biology of HCV entry into cells susceptible to infection by HCV, (See, e.g., reference 7) and serve as an authentic source of native, fusogenic forms of HCV envelope glycoproteins. HCVpp also provide a means by which to assess HCV entry into cells and to screen small molecule compounds for inhibitory activity. The findings obtained using HCVpp have been substantiated using authentic HCV (12-15).

HCVpp entry into liver cells requires co-expression of both the E1 and E2 HCV envelope glycoproteins; neither individual protein is sufficient for entry. Similar to authentic HCV and related viruses, HCVpp fusion does not occur at the cell surface but rather requires endocytosis of virus into mildly acidic endosomes, where fusion is triggered by exposure to low pH (7,16). HCVpp have been shown to be specifically inhibited by monoclonal antibodies directed against E2, as well as by HCV patient sera (7-8, 17-18). Studies with HCVpp have identified the presence of naturally-occurring, broad and cross-genotype neutralizing antibodies in sera from HCV-infected individuals (16-18).

HCVpp infect CD81-positive primary hepatocytes and liver cell lines, and monoclonal antibodies directed against CD81 inhibit HCVpp infection (6-8, 19-20). CD81-negative human hepatoma cells are resistant to HCVpp entry, but such cells become permissive when modified to express CD81. In contrast, non-hepatic cells are resistant to infection regardless of CD81 expression. Thus, CD81 expression is necessary but not sufficient for HCVpp to enter target cells. It has been demonstrated that CD81 functions as a post-attachment co-receptor for HCV as shown by the potent inhibitory activity of CD81 monoclonal antibodies added to HCVpp that were pre-bound to target cells (6). In addition, certain mutations in E2 abolish binding to CD81 but not to target cells (5, 21).

Currently, there is no vaccine against HCV and licensed antiviral therapies, such as pegylated interferon (IFN)-α and ribavirin, are associated with modest efficacies and significant toxicities. Roughly 15% of infected individuals clear the virus, and approximately 170 million people worldwide are persistently infected with HCV. The majority of these individuals may remain asymptomatic or develop chronic hepatitis or cirrhosis, which often leads to hepatocellular carcinoma. Thus, HCV infection is the cause of significant long term morbidity and mortality. In view of the prevalent and insidious nature of HCV infection in the US, as well as in other parts of the world, new, potent, more specific and effective inhibitors of HCV are needed and would be highly beneficial for treating and preventing HCV infection, as well as diseases that are caused by, or associated with, infection by HCV.

### SUMMARY

The present invention provides compounds of formula (I), pharmaceutically acceptable salts thereof, polymorphs, hydrates, stereoisomers, or prodrugs thereof:

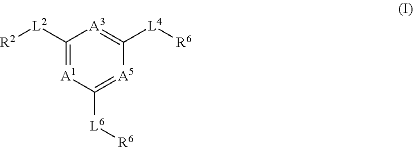

wherein,
A¹, A³, A⁵ are N;
L², L⁴, and L⁶ are independently H, O, S, NHR^{m}, (CH₂)ₙ, CN, CRR′, SO₂, CO, CONR′R, NHCONR′R, halide, cycloalkyl, heterocycle, aryl, alkyne, alkene, wherein n=0-5, and m is independently 2,4,6;
R², R⁴, and R⁶ are independently none, R, OR, amino, amine, (CH₂)ₙCF₃, CF₃, CH₂CF₃, (CH₂)ₙW, alkyl, aryl, phenyl, cycloalkyl, piperidinyl, heterocycle, fused aryl, alkylaryl, or heteroalkylaryl, all of which are optionally substituted with 0-5 W, wherein n is chosen independently to be 0-5;
W is H, halide, OR, CF₃, NO₂, CN, SO₂NRR′, SO₂R, amino, amine, aniline, ester, amide, sulfonamide, sulfamoyl, sulfone, amino acid, ether, urea, acid, heterocycle, heteroaromatic, alkyl, aryl, arylalkyl, alkylaryl, sulfone, sulfonamide substituted with alkyl, aryl, heterocycle, amino, aniline; and
R or R′ are independently H, alkyl, aryl, amide.

The invention further provides a compound of formula (Ia), its salts, including pharmaceutically acceptable salts, polymorphs, hydrates, stereoisomers, or prodrugs thereof:

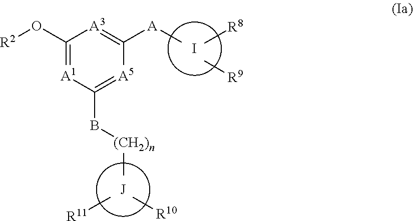

wherein:
A¹, A³, A⁵ are N;
I and J are independently, (un)substituted cycloalkyl, phenyl, aryl, piperidinyl, or heterocyclic rings of 3 to 10 carbon atoms, containing 0 to 3 heteroatoms;
A and B are independently O, S, CHR, NZ, CF₃, or bond;
n=0-5;
R⁷ is selected from H, (un)substituted alkyl, CF₃CH₂, CF₃, haloalkyl, (un)substituted aryl, (un)substituted heterocycle;
R⁸ is selected from H, OH, halo, amino, acid, ester, substituted amino, cyano, CF₃, halide, amide, acid, ester, sulfonamide, urea, (C₁-C₅) alkyl, (C₁-C₅) haloalkyl, (C₄-C₁₀) alkylcycloalkyl, (C₁-C₅) alkenyl, substituted (C₃-C₈) heterocycle of 0-3 heteroatoms;
R⁹ is selected from H, OH, halo, amino, substituted amino, cyano, amide, sulfonamide, sulfone, urea, halide, CF₃, CF₃, (C₁-C₅) alkyl, (C₁-C₅) haloalkyl, (C₄-C₁₀) alkylcycloalkyl, (C₁-C₅) alkenyl, substituted (C₃-C₈) heterocycle of 0-3 heteroatoms; further, R⁸ and R⁹ may be joined to form one or more substituted (C₃-C₈) heterocycles of 0-3 heteroatoms;
R is selected from H, alkyl, heteroaryl;
Z is selected from H, alkyl, heteroaryl; and
R¹⁰ and R¹¹ are independently selected from H, alkyl, aryl, alkylcycloalkyl, substituted alkylsulfonyl, substituted arylsulfonyl, haloalkyl, substituted heterocycle, (un)substituted alkyl or aryl, amide, sulfonamide, sulfone, urea, wherein substituents include —CO-alkyl, —CO-cycloalkyl, —CO-cycloc₅H₉, and —CO-cycloNC₄H₈.

The invention further provides compounds of formula (Ib), its pharmaceutically acceptable salts, polymorphs, hydrates, stereoisomers, or prodrugs thereof:

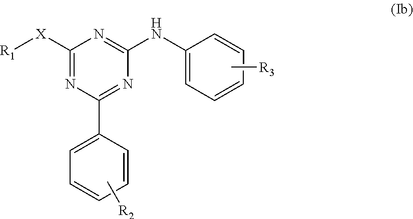

wherein:
X=H, O, NH, CH₂, halide, none;
R₁=H, OH, CF₃(CH₂)ₙ, (un)substituted alkyl or aryl, CN, CF₃;
R₂=halide, CF₃, CN, amide, amine, sulfonamide, (un)substituted alkyl or aryl, hetero ring;
R₃=H, (mono or bis) halide, CF₃, OR, amine, amide, sulfonamide, fused alkyl or aryl ring, hetero ring, (un)substituted alkyl or aryl; and
n=0-5.

The invention further provides compounds of formula (Ic), its pharmaceutically acceptable salts, polymorphs, hydrates, stereoisomers, or prodrugs thereof.

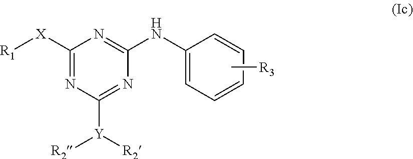

wherein:
X=H, O, NH, CH₂, halide, none;
Y=N, O(R₂″ is none), CH, alkene, alkyne;
R₁=H, OH, CF₃(CH₂)ₙ, (un)substituted alkyl or aryl, CN, CF₃;
R₂′, R₂″, independently, is H, (un)substituted alkyl, aryl, (CH₂)R, (CH₂)Ar, (CH₂)ᵣ, SO₂NRR, or together form a (un)substituted hetero ring;
R₃=H, (mono or bis) halide, CF₃, OR, amine, amide, sulfonamide, fused alkyl or aryl ring, hetero ring, (un)substituted alkyl or aryl; and
n=0-5.

The invention also further provides compounds of formula (Id), its pharmaceutically acceptable salts, polymorphs, hydrates, stereoisomers, or prodrugs thereof

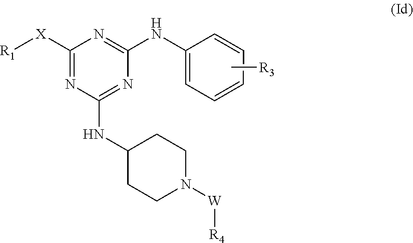

wherein:
X=H, O, NH, CH₂, halide, none;
R₃=H, (mono or bis) halide, CF₃, OR, amine, amide, sulfonamide, fused alkyl or aryl ring, hetero ring, (un)substituted alkyl or aryl;
R₄=(un)substituted alkyl, aryl, amine;
W=SO₂, CO, CH₂, none; and
n=0-5.

Additionally is provided by the invention is a compound of formula (Ie), its pharmaceutically acceptable salts, polymorphs, hydrates, stereoisomers, or prodrugs thereof

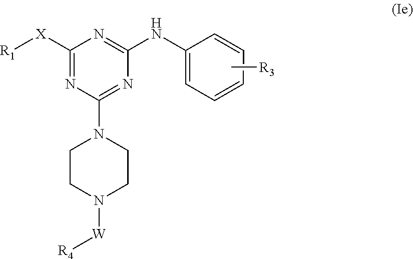

wherein:
X=H, O, NH, CH₂, halide, none;
R₁=H, OH, CF₃(CH₂)ₙ, (un)substituted alkyl or aryl, CN, CF₃;
R₃=H, (mono or bis) halide, CF₃, OR, amine, amide, sulfonamide, fused alkyl or aryl ring, hetero ring, (un)substituted alkyl or aryl;
R₄ (un)substituted alkyl, aryl, amine;
W=SO₂, CO, CH₂, none; and
n=0-5.

Also is provided by the invention is a compound of formula (If), its pharmaceutically acceptable salts, polymorphs, hydrates, stereoisomers, or prodrugs thereof

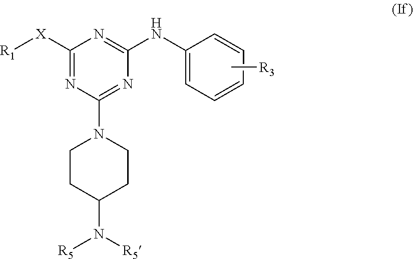

wherein:
X=H, O, NH, CH₂, halide, none;
R₁=H, OH, CF₃(CH₂)ₙ, (un)substituted alkyl or aryl, CN, CF₃;
R₃=H, (mono or bis) halide, CF₃, OR, amine, amide, sulfonamide, fused alkyl or aryl ring, hetero ring, (un)substituted alkyl or aryl;
R₅, R₅¹, independently, is (un)substituted alkyl, aryl, sulfonamide, amide; and
n=0-5.

Also provided by the present invention are at least one of a compound, stereoisomer, hydrate, polymorph, prodrug, or a salt thereof, from the exemplary compounds listed in Tables 1 to 44.

Further provided by the present invention are compositions comprising at least one of a compound, stereoisomer, hydrate, polymorph, prodrug, or a salt thereof, from the group comprising the exemplary compounds listed in Tables 1 to 44.

The invention also provides prodrugs, pharmaceutically acceptable salts, radioisomers, stereoisomers, hydrates, solvates, and acid hydrates of the compounds of the invention for use in the methods and compositions described herein. For example, prodrugs may enhance a number of desirable pharmaceutical qualities (e.g., solubility, bioavailability, manufacturing, etc.). Prodrugs of the compounds of the invention may be prepared by modifying functional groups present in the compound in such a way that the modifications are cleaved, either in routine manipulation or in vivo, to the parent compound.

The compounds are also useful as research or diagnostic reagents, as radioisomers or otherwise, whereby such compounds can be used, for example, to establish competitive binding constants for other compounds, or, for example, as quantitative reagents to assess viral titer. The compounds may be radiolabeled using radioisotopes for use alone or in a composition.

The invention further provides methods of synthesis for compounds of the invention, including salts, pharmaceutically acceptable salts, radioisomers, stereoisomers, hydrates, solvates, and acid hydrates of the compounds of the invention.

The present invention further provides a pharmaceutical dosage unit composition comprising a pharmaceutical carrier and a therapeutically effective amount of one or more compounds of the invention suitable for treating viral infections of the family Flaviviridae, and in particular, HCV infection.

The invention further provides a composition comprising a compound of the invention, or one or more compounds of the invention, and a carrier, diluent, or excipient. In an embodiment, the composition is a pharmaceutical composition and the carrier, diluent, or excipient is a pharmaceutically acceptable carrier, diluent, or excipient.

The invention also provides a method for prophylactically preventing or diminishing HCV infections in warm-blooded animals, which comprises administering before, soon after or during the exposure of said animal to HCV, a prophylactically effective amount of a compound of the invention.

In accordance with the present invention, the inventive compounds inhibit or block entry of HCV into cells that are susceptible to infection by HCV. The invention provides a method of inhibiting HCV infection of a cell susceptible to HCV infection, comprising contacting the cell with a compound of the invention in an amount effective to inhibit HCV infection of the cell. It will be generally understood that one or more compounds of the invention may be suitable for use in the methods and compositions described herein.

Also provided by the present invention is a method for treating or preventing infection by a virus of the family Flaviviridae, comprising administering to a patient in need thereof a compound or composition of the invention in an amount effective to treat or prevent the infection. In an embodiment, HCV infection is treated by administering an effective amount of one or more compounds of the invention to a patient in need thereof. In an embodiment, HCV infection is prevented by administering an effective amount of one or more compounds of the invention to a patient in need thereof. In an embodiment, HCV infection is reduced or diminished by administering an effective amount of one or more compounds of the invention to a patient in need thereof.

The invention further provides a method of reducing the occurrence of HCV infection in a population of individuals, comprising administering to the population of individuals in need thereof a compound and/or composition of the invention in an amount effective to reduce the occurrence of HCV infection in the population.

The invention also provides a method of reducing exposure of a subject to HCV infection outside or on the external body surface of the subject, comprising contacting the outside or external body surface of the subject with a compound of the invention in an amount effective to inactivate or inhibit the virus so as to reduce exposure of the subject to HCV infection.

The invention also provides a method of treating or preventing a liver disease in a subject, which comprises administering to the subject a compound and/or composition of the invention, in an amount effective to inhibit infection of the subject's HCV susceptible cells, thereby treating or preventing the liver disease in the subject.

The invention additionally provides a method of treating or preventing an HCV associated disorder in a subject, which comprises administering to the subject a compound and/or composition of the invention, in an amount effective to inhibit infection of the subject's HCV susceptible cells, thereby treating or preventing the liver disease in the subject.

Also provided by the invention is a method of inactivating, inhibiting, decontaminating, or rendering inactive or weakly infective spaces, objects, surfaces, or substances that have been contaminated with Flaviviridae viruses such as Hepatitis C Virus by bringing the spaces, objects, surfaces or substances in contact with an effective amount of a compound of the invention to accomplish inactivating, inhibiting, decontaminating, or rendering inactive or weakly infective the Hepatitis C Virus.

In an embodiment of the invention, when a viral infection is being treated, or prophylaxis is desired, a compound of the invention, or a composition containing a compound of the invention, may be administered by any route of administration, including, without limitation, intravenously, parenterally, subcutaneously, intramuscularly, orally, and as further described herein. When oral administration is utilized, a compound of the invention may be formulated to provide an immediate release dosage form that predominantly releases compound in the stomach. Alternatively, a compound of the invention may be coated to provide an enteric dosage form designed to preferentially release in the intestine, with little or no release in the stomach. Doses of the compound or the composition may be determined by routine skill and knowledge of those skilled in the pertinent art.

The invention further provides a method for inhibiting infection of a susceptible cell, wherein the cell is contacted with a compound or composition of the invention, in an amount effective to inhibit HCV infection of the cell; further, wherein the cell is in a patient and a compound of the invention is administered to the patient.

The invention provides compounds that inhibit or block entry of HCV into susceptible target cells, in one embodiment, with an EC₅₀ (half maximal effective concentration) less than or equal to 10 micromolar. In another embodiment the invention provides compounds that inhibit or block entry of HCV into susceptible target cells with an EC₅₀ less than or equal to 1 micromolar. In another embodiment the invention provides compounds that inhibit or block entry of HCV into susceptible target cells with and EC₅₀ less than or equal to 100 nanomolar. In another embodiment, the invention provides compounds that inhibit or block entry of HCV into susceptible target cells with an EC₅₀ less or equal to 50 nanomolar. In a further embodiment, the invention provides compounds that inhibit or block entry of HCV into susceptible target cells with an EC₅₀ less or equal to 10 nanomolar. In another embodiment, the invention provides compounds that inhibit or block entry of HCV into susceptible target cells with an EC₅₀ less than or equal to 5 nanomolar. In another embodiment, the invention provides compounds that inhibit or block entry of HCV into susceptible target cells with an EC₅₀ less than or equal to 1 nanomolar. In a further embodiment, the invention provides compounds that inhibit or block entry of HCV into susceptible target cells with an EC₅₀ less than or equal to 100 picomolar. In another embodiment, the invention provides compounds that inhibit or block entry of HCV into susceptible target cells with an EC₅₀ less than or equal to 10 picomolar. In yet another embodiment, the invention provides compounds that inhibit or block entry of HCV into susceptible target cells with an EC₅₀ less than or equal to 1 picomolar.

The compounds of the invention inhibit infection of susceptible cells by HCV of genotype 1. In an embodiment, the compounds of the invention inhibit infection of cells by HCV of genotype 1a. In another embodiment, the compounds of the invention inhibit infection of cells by HCV of genotype 1b. In a further embodiment, the compounds of the invention inhibit infection of cells by HCV of genotype 1a and by HCV of genotype 1b and/or other genotypes, such as genotypes 2-6 and subtypes thereof. Experiments using cloned HCV envelope glycoproteins of various 1a and 1b genotypes in the HCVpp assay, as well as in a cell culture-based HCVcc assay, were performed to determine and elucidate the HCV genotype specificity of the compounds of the invention.

The invention further provides a method of reducing or diminishing the severity of HCV infection in a subject infected or exposed to HCV comprising administering a compound of the invention to the patient in an effective amount.

Different doses of a compound of the invention may be needed depending on the infectious viral genotype. Further, different doses of a compound of the invention may be needed depending on the status of the viral infection, such that different dosages may be needed prior to potential infection, or for early post infection. Other, different dosages may be needed for obtaining a sustained viral response in the case of a long-term, chronic infection. Such doses may be determined using routine skill and methods known by those having skill in the pertinent art. To obtain therapeutic or prophylactic effects, a compound of the invention may be co-administered with one or more chemotherapeutic drugs or therapeutic or antiviral drugs or agents, or with other small molecule anti-HCV compounds. In one example, a compound of the invention may be administered with antiviral agents such as ribavirin and/or interferon-alpha (IFN-α), and in other examples, with microbial anti-infective agents or with anti-cancer agents. Administration of a compound of the invention with another drug or agent may be at the same time, or at different times. A compound of the invention may be administered to a subject at a predefined interval either prior to or subsequent to the administration of another antiviral drug, small molecule, or agent, or other therapeutic agent as described herein.

The invention also provides a method of inactivating, inhibiting, decontaminating, or rendering inactive or weakly infective objects, surfaces, or substances that have been contaminated with HCV, which comprises contacting the objects, surfaces, or substances with a compound of the invention, in an amount effective to inactivate, inhibit, decontaminate, or render inactive or weakly infective the HCV.

The compounds of the invention may be used alone as monotherapy to treat or prevent HCV infection. The compounds of the invention may also be used in combination with other antiviral drugs, including small molecules and antibodies, e.g., monoclonal, humanized, chimeric, etc. antibodies, that inhibit HCV infection. Illustratively and without limitation, such antibodies can block, prevent, disable, disrupt, or otherwise interfere with the ability of HCV to infect or reinfect cells, replicate, bind to target molecules, internalize, and the like. The compounds of the invention may further be used in combination with inactivating or decontaminating agents or drugs to render inactive or weakly infective surfaces or substances that have been contaminated with Flaviviridae such as HCV or other viruses. For instance, allograft or xenograft tissues, blood, surgical instrument surfaces, syringes, garments, and transfusion apparatuses that pose an viral infective risk to others may be rendered virally inactive or weakly infective by use of the compounds. The present invention provides a method of treating or preventing HCV infection or recurrent HCV infection in a liver transplant patient by administering a compound of the invention prior to, at the time of, or following the liver transplant. The invention further provides a method of treating or preventing HCV infection or recurrent HCV infection post-liver transplantation in patients who have undergone a liver transplant. In a nonlimiting embodiment, treatment outcome may include a decrease or reduction in viral load or viremia, or a decrease, reduction, non-detection, or absence of virus, e.g., viral particles, virions, viral nucleic acid, in the transplant patient. According to the methods of the invention, treatment of the liver transplant patient may diminish or reduce the severity of, inhibit, block or eradicate liver damage, liver fibrosis, advanced fibrosis, or cirrhosis in the patient. In an embodiment, a compound of the invention is administered to the patient prior to liver transplant. In an embodiment, a compound of the invention is administered to the patient at the time of liver transplant. In an embodiment, a compound of the invention is administered to the patient post-liver transplant. In an embodiment, a compound of the invention is administered to the patient post-liver transplant over a prolonged period of time, such as days, weeks, or months following the liver transplant. In an embodiment, a compound of the invention is administered to the patient prior to, at the time of and following liver transplantation in the patient. The methods of the invention involve administering a compound of the invention, either alone or in combination with another antiviral or anti-HCV drug, compound, therapeutic, or inhibitor, or with an HCV standard of care (SSOC) drug or therapeutic, e.g., interferon and ribavirin, in an amount effective to treat or prevent the HCV infection or recurrent HCV infection in a liver transplant patient.

### BRIEF DESCRIPTION OF FIGURES

FIG. 1 provides a schematic structural representation of the hepatitis C virus. Shown are the viral lipid bilayer (viral envelope), icosahedral core, viral RNA, and envelope glycoproteins E1 and E2.

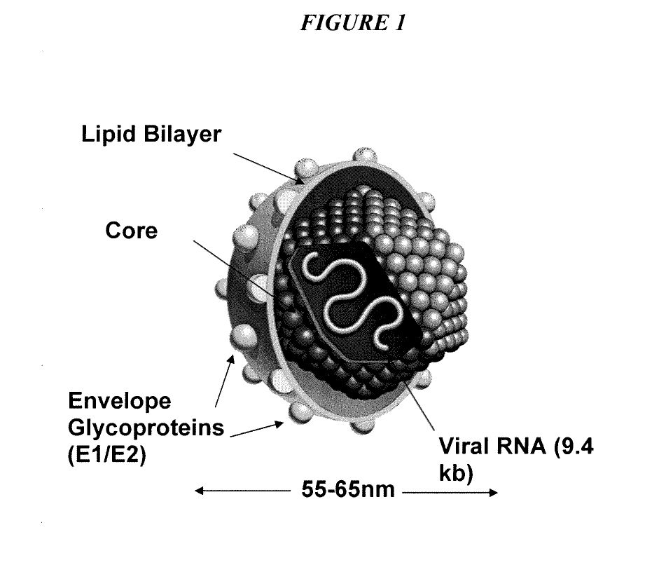

FIG. 2 provides a schematic representation of HCV-entry into a permissive liver-derived human hepatoma cell line showing cell receptor-virus binding and low pH-dependent membrane fusion with release of the viral nucleocapsid.

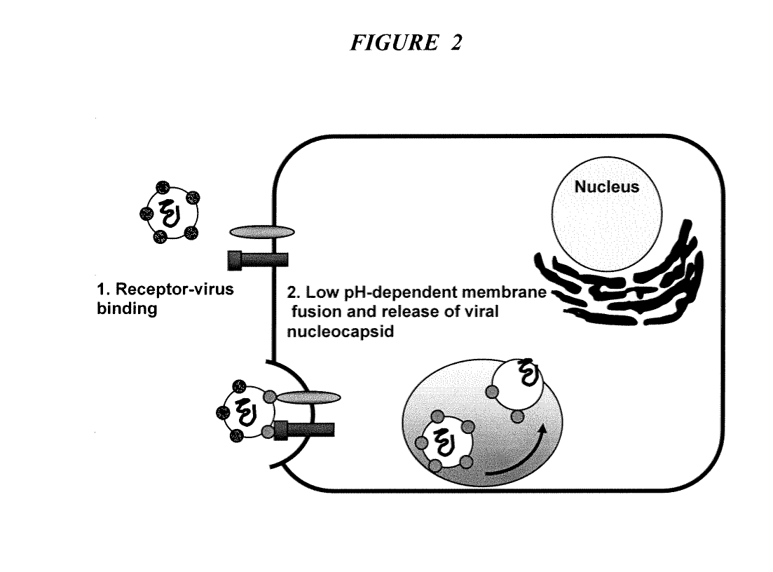

FIG. 3 depicts a schematic representation of the action of an HCV entry inhibitor. An inhibitor compound may bind to the HCV E1, E2, or E1/E2 envelope glycoproteins, or to a cell receptor or receptor complex, and inhibit attachment and/or fusion of the virus, thus inhibiting virus entry into and infection of the cell.

FIG. 4 provides a schematic representation of the HCVpp high throughput screening assay. Compounds are evaluated at a single concentration for their ability to inhibit entry of HCVpp into permissive human hepatoma cells.

FIG. 5 depicts a time-of-addition assay. Target cells were infected with HCVpp. Compounds were added at various time points (0-130 min) post-infection, and luciferase activity (RLU-relative light units) was analyzed 72 hours post-infection. Representative inhibitors, including the anti-CD81 monoclonal antibody JS-81, completely block HCVpp entry only when added during the first 60 minutes of infection.

FIG. 6 depicts a schematic representation of various HIV-1 based pseudoviral particles utilized in assays to evaluate the compounds of the invention. Test and control pseudoparticles include HCVpp, VSVpp (Vesicular Stomatitis Virus pp), MLVpp (Murine Leukemia Virus pp), HIV-1 pp, and human patient specific HCVpp. The pseudoviruses are capable of only a single round of infection in target cells. Of the pseudoparticles, HCVpp only recapitulate the process of HCV entry.

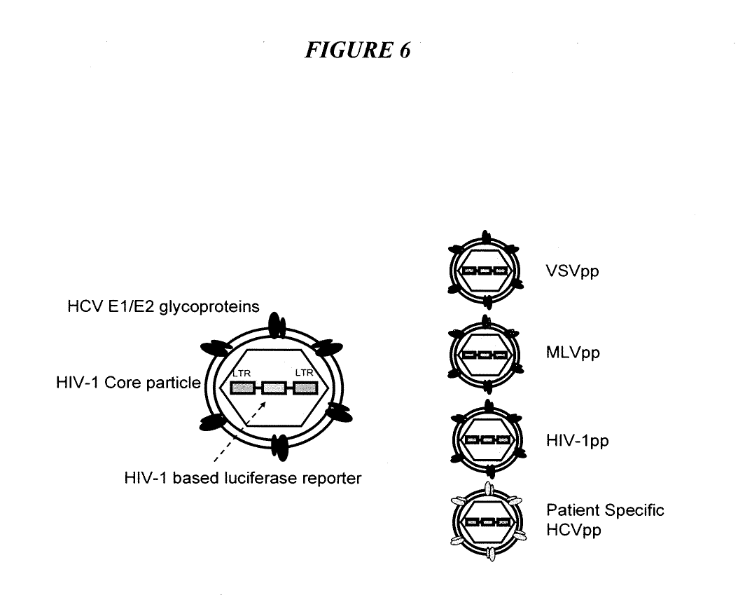

FIG. 7 demonstrates an HCVpp entry assay used to evaluate compounds of the invention. In this assay, for HCVpp: HIV-1 core particles encoding a luciferase reporter gene were pseudotyped with the E1 and E2 envelope glycoproteins from HCV by co-transfection of 293T cells with the appropriate expression constructs. Viral particles were harvested and clarified by centrifugation. HCVpp were used to infect Hep3B cells in the presence of test compounds or controls. Entry activity was determined by quantifying luciferase gene expression 72 hours post infection.

FIG. 8 demonstrates the potency of one of the HIV inhibitor compounds of the invention, designated PRO206, in the HCVpp assay. PRO206 was subjected to 0.5 log serial dilutions in DMSO. HCVpp pseudotyped with the H77 envelope were added to Hep3B cells in 384-well microplates in the presence of various concentrations of PRO206. Luciferase activity was measured 72 hours post-infection. The EC₅₀ value was 2.2 nM (n=22). Accordingly, PRO206 demonstrated potent activity against HCVpp (H77).

FIG. 9 demonstrates the antiviral efficacy of PRO206 in an HCV cell culture (HCVcc) model. H77/JFH-1 chimeric HCVcc system was used to determine the antiviral activity of the PRO206 compound. HCV particles produced in Huh-7.5 cells were harvested, clarified and used to infect naive Huh 7.5 cells in the presence of various concentrations of PRO206. The anti-CD81 MAb, JS-81, and the nucleoside analog 2-CMA were used as positive controls in the assay. Renilla luciferase activity was measured 72 hours post-infection. The antiviral activity in the HCV cell culture model was consistent with that obtained in the HCVpp-based assay. For the PRO206 compound, the EC₅₀ value was 6.9 nM; the EC₉₀ value was 31 nM. For the assay controls, the EC₅₀ value for JS-81 was 0.18 μg/mL, and the EC₅₀ value for 2-CMA was 92 nM. Thus, PRO206 exhibited potent and specific antiviral activity against HCVcc in the cell culture efficacy model.

FIG. 10 illustrates the protocol for a time-of-addition assay to investigate the mechanism of action of PRO206, an exemplary compound of the invention.

FIG. 11 depicts the results of experiments showing that the activity of PRO 206 is consistent with its being a post-attachment entry inhibitor. PRO206 (10 nM) or JS-81 (1 μg/mL) were added to Hep3B cells at different stages of the virus entry process. Pre-Treatment: PRO206 and JS-81 were pre-incubated with Hep3B cells for 2 hours at 4° C. Unbound compound was washed away with PBS and HCVpp were added. The culture was then shifted to 37° C. Co-treatment: The assay was performed in the standard format as described above for FIG. 8. Attachment: PRO206 and JS-81 were added to Hep3B cells in the presence of HCVpp at 4° C. After 2 hours, unbound virus and compound were washed away with PBS. Post-attachment: HCVpp were added to Hep3B cells at 4° C. After 2 hours, unbound HCVpp was washed away with PBS. PRO206 was added and the cultures were shifted to 37° C. to allow entry to proceed. Potency of PRO206: EC50: 0.002 μM. Potency of JS-81: EC50: 0.08 μg/mL. The time of inhibition activity of PRO206 is consistent with that of a post-attachment entry inhibitor.

FIG. 12 demonstrates a pharmacokinetic profile for PRO206 in rats. Both intravenous (IV) dosing and oral dosing were evaluated. Favorable levels of oral exposure and bioavailability (% F=34%) were observed in vivo. The PRO206 compound is predicted to achieve trough concentrations that are many multiples above the EC₅₀. The PK properties of PRO206 are supportive of exploration of once daily dosing in humans.

### DETAILED DESCRIPTION

The novel compounds of the invention inhibit HCV infection of, or HCV entry into, cells that are susceptible to infection by HCV, such as liver cells, hepatocytes and other permissive cell types. According to the invention, heterocyclic compounds, such as azines, e.g., substituted 1,3,5-triazine-2,4,6-diamines, and substituted alkoxy-triazine-2,4-diamines, were discovered to be highly active in inhibiting or blocking the entry of HCV into susceptible cells. The compounds of the invention inhibit infection of susceptible cells by HCV of genotype 1, in particular genotype 1a, genotype 1b, or both genotype 1a and 1b. The inhibitory activity of the compounds of the invention may include activity against other virions of the Flaviviridae family. The properties of the inventive compounds as described herein are highly advantageous and offer significant therapeutic benefit, illustratively because HCV of genotype 1 represents the predominant HCV genotype of the HCV infected population in the US. For example, recent analyses have reported that the HCV genotype 1a subtype represents approximately 56.7% of the HCV infected population in the US, while the HCV genotype 1b subtype represents approximately 17% of the HCV infected population in the US.

The compounds of the present invention are advantageous as potent and selective inhibitors of HCV infection of susceptible cells. The compounds of the invention are advantageous as potent and selective blockers of HCV entry into susceptible cells. Viral entry represents a novel and particularly attractive treatment class for HCV, because entry is mediated by conserved structures on the viral and cellular membranes. That the compounds of the invention target and inhibit HCV entry into susceptible cells, e.g., liver cells, is particularly advantageous because such inhibitors do not need to cross the plasma membrane or be modified intracellularly. Consequently, inhibitors of viral entry can be very potent, broadly active and present a higher barrier to viral resistance.

The inhibitory activity and function of the disclosed compounds can be assessed and measured in a Hepatitis C Virus cell culture system (HCVcc) model of HCV infection that utilizes cloned, recombinant, infectious virus and susceptible target cells, which can be infected by the cloned virions. Nonlimiting examples of HCVcc include those such as are described, for example, in U.S. Pat. Nos. 5,874,565; 6,127,116 and 7,235,394 to C. M. Rice et al.; WO08/024,413 to Novartis AG; WO91/02820 to Chiron Corp. Such HCV cell culture systems involve an authentic HCV nucleic acid (e.g., DNA, cDNA or RNA) clone that is capable of replication in an appropriate cell line, expression of functional viral proteins and infection of cells. Such an HCV nucleic acid clone is genetically engineered and contains the appropriate genetic machinery for replication, virion production and infection of cells, including polyprotein coding sequences from one or more HCV genotypes, e.g., HCV-1, HCV-1a, HCV-1b, HCV-1c, HCV-2a, HCV-2b, HCV-2c, HCV-3a, or quasi-species and variants thereof. The HCV clones typically contain an adaptive mutation that allows for higher levels of HCV replication in the cell line. The HCV clones may also be chimeric and encode proteins of two or more different genotypes, e.g., 1a/2a; 1a/2b; 1b/2a, and the like. Recently, chimeric full-length constructs containing the nonstructural proteins of JFH-1 and the structural proteins of genotype 1 clones such as H77C, J4 or Coni have also become available (Gottwein, J. M. et al., 2008. “Development and characterization of hepatitis C virus genotype 1-7 cell culture systems: Role of CD81 and SR-BI and effect of antiviral drugs”, Hepatology 9999:999 A; Scheel, T. K. et al., 2008., “Development of JFH1-based cell culture systems for hepatitis C virus genotype 4a and evidence for cross-genotype neutralization”, Proc Natl Acad Sci USA, 105:997-1002; Zhang, Y., et al., 2008, “Novel chimeric genotype 1b/2a hepatitis C virus suitable for high-throughput screening”, Antimicrob Agents Chemother, 52:666-674). These chimeric HCVcc systems provide unique tools to determine the antiviral activity, mechanism of action and determinants of drug resistance for inhibitors of HCV entry in a genotype 1 context.

The inhibitory activity of a compound of the invention can be analyzed in an HCVcc system, for example, by contacting a cell line infected by an infectious HCV RNA and assaying for an increase or decrease in level of HCV infection or activity compared with a level of HCV infection or activity in a control cell line, or in the cell line prior to administration of the compound. A decrease in the level of HCV infection or activity compared with the level of HCV infection or activity in a control cell line or in the cell line prior to addition of the compound is indicative of the ability of the compound to inhibit HCV infection or activity. Testing for the level of HCV infection in such a system can be accomplished by measuring viral titer in the cells, culture medium, or both; and/or measuring viral proteins in the cells, culture medium, or both. In another aspect, the HCV genome used to infect the cell line may contain a heterologous gene operatively associated with an expression control sequence, in which the heterologous gene and expression control sequence are oriented on the positive-strand nucleic acid molecule. In such a case, testing for the level of HCV activity involves measuring the level of a marker protein, e.g., in a tissue sample from the subject. Additionally, HCVcc systems involving a cloned reporter HCV, which provides a read-out, such as enzyme activity, e.g., luciferase, are conveniently employed using techniques known in the art.

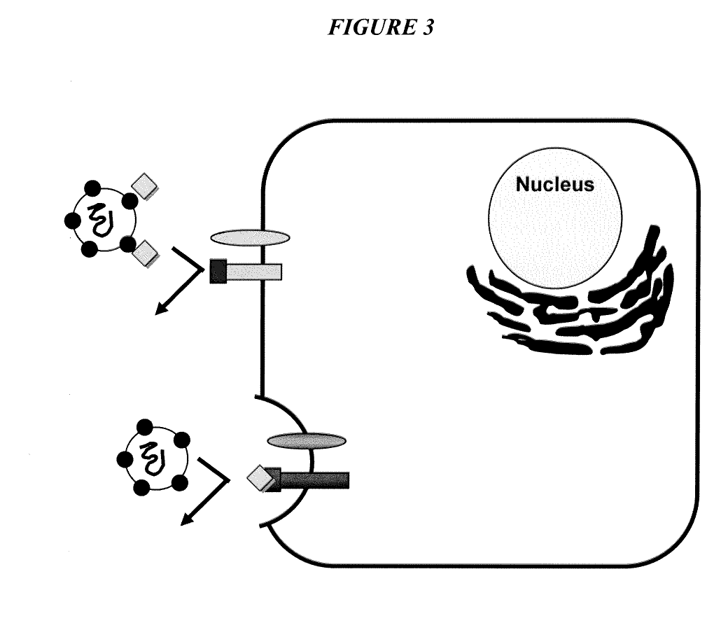

Without wishing to be bound by theory related to a mechanism of receptor blockade, the blocking mechanism of the compounds may involve blocking or inhibiting a direct interaction of HCV with its receptor on a susceptible cell. For example, the interaction may involve ectopic domain of the CD81 receptor. Alternatively, a direct binding of the compounds with the viral envelope glycoproteins E1, E2, or E1/E2 may occur, such that virion docking or fusion is impeded. (FIG. 3). In a different modality, prior to the release of newly-formed viral replicons, the compounds may also potentially block the assembly and/or facile release of viral particles from infected cells via intracellular binding to newly formed E1/E2 structural proteins. In this therapeutic modality, the compounds would function as virion assembly or exit inhibitors. Without wishing to be bound by theory, the compounds of the present invention may function as post-attachment viral entry inhibitors.

The invention further provides the stereoisomers of the compounds disclosed herein, as well as to prodrugs, polymorphs, solvates, all salts thereof, particularly pharmaceutically acceptable salts, synthetic methods for the preparation of compounds of the invention, pharmaceutical compositions of the same, and methods for therapeutic and/or prophylactic utilization, preparation, and pharmaceutical compositions.

The invention also provides methods for utilizing these compounds in anti-viral treatment, therapy, or prophylaxis, either as monotherapy or in combination with other antiviral or chemotherapeutic and/or prophylactic agents. Further, the invention provides for any human and/or animal subject or patient that may be treated with compounds according to the invention.

In this application it will be understood that the terms “therapeutic” and “therapy” are used to describe the administration of medicaments to a subject or patient to treat, reduce, diminish, correct, ameliorate, or eradicate an infection, condition, or pathology that has already initiated. The terms “prophylactically” and “prophylaxis” describe protective medications or preventive treatments that are administered to a subject and/or applied to an object before contact with HCV, for example, to prevent, reduce, or diminish the intensity or severity of a subsequent infection of the patient by the virus, or to prevent, reduce, or diminish contamination of the object by the virus.

The term “acyl”, whether used alone, or within a term such as “acylamino”, denotes a radical provided by the residue after removal of hydroxyl from an organic acid. The term “acylamino” embraces an amine radical substituted with an acyl group. An example of an “acylamino” radical is acetylamine (CH₃C(═O)—NH—). The term “aryloxy” denotes a radical provided by the residue after removal of hydrido from a hydroxy-substituted aryl moiety (e.g., phenol).

As used herein, “alkanoyl” refers to a-C(═O)-alkyl group, wherein alkyl is as previously defined. Exemplary alkanoyl groups include acetyl (ethanoyl), n-propanoyl, n-butanoyl, 2-methylpropanoyl, n-pentanoyl, 2-methylbutanoyl, 3-methylbutanoyl, 2,2-dimethylpropanoyl, heptanoyl, decanoyl, and palmitoyl.

The term “alkenyl” includes unsaturated aliphatic groups analogous in length and possible substitution to the alkyls described below, but that contain at least one double bond and must contain at least two carbon atoms. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “lower alkylene” herein refers to those alkylene groups having from about 1 to about 6 carbon atoms. The term “alkenyl” includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkenylene”, in general, refers to an alkylene group containing at least one carbon-carbon double bond. Exemplary alkenylene groups include, for example, ethenylene (—CH═CH—) and propenylene (—CH═CHCH₂—). Preferred alkenylene groups have from 2 to about 4 carbons.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro chloro or bromo to provide “haloalkoxy” or “haloalkoxyalkyl” radicals. Examples of “alkoxy” radicals include methoxy butoxy and trifluoromethoxy.

“Alkyl” in general, refers to an aliphatic hydrocarbon group which may be straight, branched or cyclic having from 1 to about 10 carbon atoms in the chain, and all combinations and subcombinations of ranges therein, e.g., a cycloalkyl, branched cycloalkylalkyl, a branched alkylcycloalkyl having 4-10 carbon atoms. The term “alkyl” includes both “unsubstituted alkyls” and “substituted alkyls,” the latter of which refers to alkyl moieties having substituents replacing a hydrogen on one or more carbons of the backbone. “Lower alkyl” refers to an alkyl group having 1 to about 6 carbon atoms. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2-dimethylbutyl, and 2,3-dimethylbutyl, cyclopropylmethyl and cyclobutylmethyl. Alkyl substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. The term “aralkyl” embraces aryl-substituted alkyl radicals such as benzyl, diphenylmethyl, triphenylmethyl, phenethyl, phenylpropyl, and diphenethyl. The terms benzyl and phenylmethyl are interchangeable. The term “n-alkyl” means a straight chain (i.e. unbranched) unsubstituted alkyl group. “Branched” refers to an alkyl group in which a lower alkyl group, such as methyl, ethyl or propyl, is attached to a linear alkyl chain.

The term “alkynyl” includes unsaturated aliphatic groups analogous in length and possible substitution to the alkyls described above, but which contain at least one triple bond and two carbon atoms. For example, the term “alkynyl” includes straight-chain alkynyl groups (e.g., ethynyl, propynyl, butynyl, pentynyl, hexynyl, heptynyl, ° C.tynyl, nonynyl, decynyl, etc.), branched-chain alkynyl groups, and cycloalkyl or cycloalkenyl substituted alkynyl groups.

The term “amido” when used by itself or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkyl radical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl”embraces alkyl radicals substituted with an N-alkyl-N-hydroxyamido radical. The term “amidoalkyl” embraces alkyl radicals substituted with amido radicals.

The terms “amine” or “amino” have their common, ordinary meaning. In general, the amines useful in the invention have the general formula:

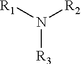

wherein R₁, R₂, and R₃ are identical or a combination of different hydrido, straight or branched chain alkyl groups, alkenyl groups, alkylene groups, alkenylene groups, cycloalkyl groups, cycloalkyl-substituted alkyl groups, cycloalkenyl groups, alkoxy groups, alkoxy-alkyl groups, acyl groups, aryl groups, aryl-substituted alkyl groups, and heterocyclic groups, such as morpholine. If none of R₁₋₃ are hydrido, the compound is a tertiary amine. Exemplary tertiary amines useful according to the invention are those where R₁₋₃ is an alkyl group of the formula (CₙH₂ₙ₊₁, n=1-4), or aralkyl group of the formula (C₆H₅ (CH₂)ₙ— [n=1-2]. Exemplary tertiary amines useful according to the invention also are cycloalkyl tertiary amines (e.g., N-methylmorpholine, N-methylpyrrolidine, N-methylpiperidine), pyridine and Proton Sponge® (N,N,N′,N′-tetramethyl-1,8-naphthalene).

The term “aminoalkyl” embraces alkyl radicals substituted with amine radicals. The term “alkylaminoalkyl” embraces aminoalkyl radicals having the nitrogen atom substituted with an alkyl radical. The term “amidino” denotes an —C(═NH)—NH₂ radical. The term “cyanoamidino” denotes an —C(═N—CN)—NH₂ radical.

The term “anti-HCV compound” refers to any compound showing the effect of inactivating HCV or inhibiting, blocking, or diminishing infectivity or replication of the virus in any way. One possibility for anti-HCV activity, for example, is a compound that interferes with the entry of HCV into an animal cell; such a compound is an “entry inhibitor”. If such a compound interferes with the exit of viral replicons from the cell, after infection by the virus, the compound is an “exit inhibitor”. A third possibility is a compound that enhances the effectiveness of the subject's immune system in attacking and neutralizing the virus. Yet another possibility, for example, is a compound that interferes with the viral life cycle once the virus has gained cellular entry. An example of such a compound is a “HCV-metalloprotease” inhibitor which inhibits the virus' metalloprotease, a viral enzyme that is thought to cleave the viral polypeptide at its NS2/NS3 junction. Another example is an “HCV polymerase” inhibitor which inhibits the HCV encoded RNA dependent RNA polymerase (known as NS5B) that the virus needs in order to replicate its genome. The viral HCV polymerase, NS5B, is essential for viral replication. Yet another example is an “HCV serine protease” inhibitor. Such a compound interferes with the virally encoded serine protease known as NS3/4A that is essential for viral polypeptide cleavage. And yet another such compound is an “HCV helicase” inhibitor which prevents the unwinding of the viral genome by interfering with the enzyme HCV-helicase encoded by the virus.

“Anti-HCV monoclonal antibodies” are antibodies that are reactive toward HCV. Monoclonal antibodies are identical in their binding specificity, having been produced by B cells that are all genetically identical clones of a single parent B cell. “Anti-HCV polyclonal antibodies” are antibodies that are reactive against HCV. Such antibodies are produced from different B cells, and are a mixture of different immunoglobulin molecules, each of which recognizes a specific antigenic site or epitope on the virus.

The term “anti-infective agent” refers to a compound, composition, substance, reagent, drug, and the like, which acts therapeutically or prophylactically against infectious viral (e.g. HCV), bacterial, protozoal, or other agents by inhibiting their growth, replication, and survival. Anti-infective agents may comprise preparations that contain natural or synthetic antibiotic agents.

The term “anti-cancer agent” or “cancer chemotherapeutic agent” refers to a compound, composition, substance, reagent, drug, and the like, which acts therapeutically or prophylactically by inhibiting the growth, replication, spread, and survival of cancer cells. Anti-cancer agents may comprise preparations that contain natural or synthetic materials that act therapeutically singly or in combination to achieve their effect.

The term “antisense molecule” refers to a nucleic acid molecule (DNA, RNA, or a chemical analogue) that will complementarily bind to viral RNA, thus preventing the translation of viral proteins, thereby interfering with the viral life cycle. More generally, an antisense molecule binds to or pairs with messenger RNA (mRNA), e.g., an mRNA transcript, to block the expression of a gene, thus effectively turning off that gene and inhibiting its function. The interfering molecule, typically an oligonucleotide, is termed “antisense” because its base sequence is complementary to the RNA, i.e., the “sense” sequence.

The term “aryl”, alone or in combination, means a carBocyclic aromatic system containing one, two or three rings wherein such rings may be attached together in a pendent manner or may be fused. The term “aryl” embraces aromatic radicals such as phenyl, naphthyl, tetrahydronapthyl, indane and biphenyl.

“Aryl-substituted alkyl”, in general, refers to an linear alkyl group, preferably a lower alkyl group, substituted at a carbon with an optionally substituted aryl group, preferably an optionally substituted phenyl ring. Exemplary aryl-substituted alkyl groups include, for example, phenylmethyl, phenylethyl and 3-(4-methylphenyl)propyl.

As used herein, the term “associated liver disorder” refers to any liver dysfunction, pathology, or malady associated with infection by a virus of the Flaviviridae family, in one example HCV.

The term “cycloalkyl” embraces radicals having three to ten carbon atoms, such as cyclopropyl cyclobutyl, cyclopentyl, cyclohexyl and cycloheptyl.

The term “carbocycle” is intended to mean any stable 3- to 7-membered monocyclic or bicyclic or 7- to 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, or tetrahydronaphthyl (tetralin). Preferred “carbocycle” are cyclopropyl, cyclobutyl, cyclopentyl, and cyclohexyl.

“Cycloalkyl-substituted alkyl”, in general, refers to a linear alkyl group, preferably a lower alkyl group, substituted at a terminal carbon with a cycloalkyl group, preferably a C₃-C₈ cycloalkyl group. Typical cycloalkyl-substituted alkyl groups include cyclohexylmethyl, cyclohexylethyl, cyclopentylethyl, cyclopentylpropyl, cyclopropylmethyl and the like.

“Cycloalkenyl”, in general, refers to an olefinically unsaturated cycloalkyl group having from about 4 to about 10 carbons, and all combinations and subcombinations of ranges therein. In some embodiments, the cycloalkenyl group is a C₅-C₈ cycloalkenyl group, i.e., a cycloalkenyl group having from about 5 to about 8 carbons.

The term “halo” means halogens such as fluorine, chlorine, bromine or iodine atoms. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either a bromo, chloro or a fluoro atom within the radical. Dihalo radicals may have two or more of the same halo atoms or a combination of different halo radicals and polyhaloalkyl radicals may have more than two of the same halo atoms or a combination of different halo radicals.

As used herein, the term “heterocycle” or “heterocyclic ring” or “heterocycloalkyl ring” is intended to mean a stable 5- to 7-membered monocyclic or bicyclic or 7- to 14-membered bicyclic heterocyclic ring which is saturated, partially unsaturated, or unsaturated (aromatic), and which consists of carbon atoms and 1, 2, 3 or 4 heteroatoms independently selected from the group consisting of N, O and S and including any bicyclic group in which any of the above-defined heterocyclic rings is fused to a benzene ring. Examples of saturated heterocyclic radicals include pyrrolidinyl, piperidinyl, and morpholinyl.

The term “hydroxyalkyl” embraces linear or branched alkyl radicals having one to about ten carbon atoms any one of which may be substituted with one or more hydroxyl radicals.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

The term “interferon” denotes a natural protein produced by the cells of the immune response of most vertebrates when challenged by foreign agents such as viruses, bacteria, parasites and tumor cells. There are different types of interferons (e.g., interferon-alpha, interferon-beta, interferon-gamma), which belong to the class of glycoproteins known as cytokines. Interferons are biological defense modifiers, which inhibit viral replication within cells of the body and thereby assist immune response, e.g., the eradication of virus and viral infection. Interferons are antiviral and anti-oncogenic, assist macrophage and natural killer lymphocyte activation, and enhance major histocompatibility complex glycoprotein classes I and II, and thereby the presentation of foreign (microbial) peptides to T cells, which have immune effector function to combat infection.

The term “liver disease” refers to any pathology, dysfunction, condition, illness, inflammation, cancer or malady of the liver. Non-limiting examples are amebic liver abscess, autoimmune hepatitis, biliary atresia, cirrhosis, dessiminated coccidioido-mycosis, delta agent (hepatitis D), drug-induced cholestasis, hemochromatosis, hepatitis A, hepatitis B, hepatitis C, hepatocellular carcinoma, liver disease due to alcohol, primary biliary cirrhosis, pyogenic liver abscess. Reye's syndrome, sclerosing cholangitis, and Wilson's disease.

The terms “N-alkylamino” and “N,N-dialkylamino” denote amine groups which have been substituted with one alkyl radical and with two alkyl radicals, respectively.

“Organic solvent” has its common ordinary meaning to those of skill in the art. Exemplary organic solvents useful in the invention include, but are not limited to, tetrahydrofuran, acetone, hexane, ether, chloroform, acetic acid, acetonitrile, chloroform, cyclohexane, methanol, and toluene. Anhydrous organic solvents are included.

As used herein, “patient” or “subject” refers to humans and to animals, including mammals, e.g., rodents (mice, rats) dogs, rabbits, sheep, goats, and non-human primates. The term “patient” refers preferably to humans.

As used herein, “prodrug” refers to compounds specifically designed to maximize the amount of active species that reaches the desired site of reaction that are of themselves typically inactive or minimally active for the activity desired, but through biotransformation or chemical reaction are converted into biologically active metabolites.

As used herein, “pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problem complications commensurate with a reasonable benefit/risk ratio. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, ethylenediaminetetraacetic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Certain acidic or basic compounds of the present invention may exist as zwitterions. All forms of the compounds, including free acid, free base and zwitterions, are contemplated to be within the scope of the present invention. It is well known in the art that compounds containing both amino and carboxyl groups often exist in equilibrium with their zwitterionic forms. Thus, any of the compounds described herein throughout that contain, for example, both amino and carboxyl groups, also include reference to their corresponding zwitterions.

The term “ribozyme”, derived from a contraction of ribonucleic acid enzyme, refers to a RNA molecule that catalyzes a chemical reaction, typically either the hydrolysis of one of its' own phosphodiester bonds, or the hydrolysis of bonds in other RNAs. Ribozymes are naturally occurring or synthetic. Non-limiting examples of naturally occurring ribozymes are Peptidyl transferase 23S rRNA, Rnase P, GIR1 branching ribozyme, Hairpin ribozyme, Hammerhead ribozyme, HDV ribozyme, Mammalian CPEB3 ribozyme, VS ribozyme, glmS ribozyme and CoTC ribozyme.

As used herein, a “susceptible cell” is a cell which is subject to and/or permissive to infection by a virus, in one example, HCV. HCV may enter and infect a susceptible cell. A susceptible may also be referred to as a target cell.

As used herein, the term “virion” refers to a mature virus, such as a mature virus particle, either existing outside a cell, or nascent within a cell prior to release. The subjects or patients to which the compounds of the present invention may be administered are vertebrates, in particular mammals. In embodiments the mammal is a human, nonhuman primate, dog, cat, sheep, goat, horse, cow, pig or rodent. In one embodiment, the mammal is a human.

The pharmaceutical preparations or compositions of the invention, when used alone or in cocktails, are administered in therapeutically effective amounts. An effective amount will be determined by the parameters discussed below; but typically is that amount which establishes a level of the drug(s) effective for treating a subject, such as a human subject, having one of the conditions described herein. An effective amount means that amount alone, as a single dose, or as multiple doses, necessary to delay or prevent the onset of, lessen the severity of, inhibit completely, lessen or reduce the progression of, eradicate, or halt altogether the onset or progression of the condition being treated or a symptom associated therewith. In the case of an active viral infection, an effective therapeutic amount, for example, is that amount which eliminates viral infection, eradicates viral infection, relieves a symptom of infection, causes or induces a decrease in viral load, increases the time before relapse, or decreases circulating viral RNA. In the case of prophylactic usage, either before transmission or soon after transmission, an effective amount, for example, would be an amount that prevents active infection, lowers the frequency of active infection, slows or reduces the time before an active infection occurs, or diminishes the intensity of the infection.

Patients amenable to the therapy of the present invention also include but are not limited to patients suffering from other dysfunctions.

A variety of routes of administration are encompassed by the invention. The particular mode selected will depend, of course, upon the particular combination of drugs selected, the severity of the condition being treated, or prevented, the condition of the patient, and the dosage required for therapy and/or efficacy. The methods of this invention, generally speaking, may be practiced using any mode of administration that is medically acceptable, meaning any mode that produces effective levels of the active compounds without causing clinically unacceptable adverse effects. Such modes of administration include, without limitation, oral, rectal, topical, transdermal, sublingual, intravenous infusion, pulmonary, intra-arterial, intra-adipose tissue, intra-lymphatic, intramuscular, intracavity, intraperitoneal (IP), intrathecal, subcutaneous (SC), aerosol, aural (e.g., via eardrops), intranasal, inhalation, intra-articular, needleless injection, subcutaneous or intradermal (e.g., transdermal) delivery. For continuous infusion, a patient-controlled device or an implantable drug delivery device may be employed. The administration may be by the patient, using an injection device for SC self-administration. Oral, rectal, or topical administration may be important for long-term treatment. Preferred rectal modes of delivery include administration as a suppository or enema wash.

The pharmaceutical preparations may conveniently be provided in unit dosage form and may be prepared by any of the methods well known in the art of pharmacy. In such form, the entire unit is intended to be administered to the patient as a separate dose. All methods include the step of bringing the compounds of the invention into association with a carrier which constitutes one or more accessory ingredients. In general, the compositions are prepared by uniformly and intimately bringing the compounds of the invention into association with a liquid carrier, a finely divided solid carrier, or both, and then, if necessary, shaping the product.

When administered, the pharmaceutical preparations of the invention are applied in pharmaceutically acceptable compositions. Such preparations may routinely contain salts, buffering agents, preservatives, compatible carriers, lubricants, and optionally other therapeutic substances and/or ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic.

It should be understood that when referring to compounds of the invention, salts of the same are encompassed. Such salts are of a variety well known to those or ordinary skill in the art. When used in pharmaceutical preparations, the salts preferably are pharmaceutically-acceptable for use in humans. A bromide salt is an example of one such salt in the case that the parent compound is basic. A sodium salt is an example of one such salt in the case that the parent compound is acidic.

It should be understood that when referring to compounds of the invention, radioisomers of the same are encompassed. Such isomers, obtained by replacing one or more component atoms of the compound by a radioactive atom, are of a variety well known to those or ordinary skill in the art. In the present case, such radioisomers can be used therapeutically to deliver localized radiation to a tissue, in one embodiment, a tissue infected with HCV; or in another example, a radioisomer may be used as a tracer to measure metabolic pathways in an animal, or to measure competitive binding in a laboratory sample of tissue. Non-radioactively labeled compounds, produced by replacing one or more of the component atoms with an atomic isotope thereof, are also encompassed.

It should also be understood that when referring to compounds of the invention, hydrates, solvates, and polymorphs of the same are encompassed. Hydrates are formed when water binds to the crystal structure of a compound in a fixed stoichiometric ratio, although generally this ratio will change depending on the surrounding humidity with which the hydrate is in equilibrium. Hydration is a more specific form of solvation. Solvates are crystalline solid adducts containing either stoichiometric or nonstoichiometric amounts of a solvent incorporated within the crystal structure. If the incorporated solvent is water, the solvates are also commonly known as hydrates. Hydrates and solvates are well known to those or ordinary skill in the art.

Polymorphism is characterized as the ability of a compound or drug substance to exist as two or more crystalline phases that have different arrangements and/or conformations of the molecules in the crystal lattice. Amorphous solids consist of disordered arrangements of molecules and do not possess a distinguishable crystal lattice. Polymorphism refers to the occurrence of different crystalline forms of the same drug substance. Polymorphs are well know to those of ordinary skill in the art.

Polymorphs or solvates of a solid can have different chemical and physical properties such as melting point, chemical reactivity, apparent solubility, dissolution rate, optical and electrical properties, vapor pressure, and density, for example. These properties can have a direct impact on the processing of drug substances and the quality or performance of drug products. Chemical and physical stability, dissolution, and bioavailability are some of these qualities. A metastable solid form may change crystalline structure or solvate or desolvate in response to changes in environmental conditions, processing, or over time. New polymorphs can develop spontaneously over time.

Infection by Hepatitis C virus predominantly occurs via the percutaneous exchange of infected blood from an outside source, such as a contaminated syringe needle. In an embodiment, after being applied in an effective amount to such a source, a compound of the disclosure is capable of reducing exposure of a patient to HCV infection. In this embodiment, an effective amount of a compound of the invention inactivates, inhibits, or diminishes infectivity of the virus outside, or on the body, of a subject after contacting virus. An effective amount would be the amount of a compound of the invention that diminishes or eliminates the infectivity of a virally contaminated outside source, upon the contact of the source with the subject.

Compounds of the invention may be used alone, i.e., as a monotherapy or mono-treatment for treating or preventing HCV infection. Additionally, compounds of the invention may be used in combination with other antivirals or other drugs, including small molecule drugs and biologics, i.e., as a combination therapy, for treating or preventing HCV infection. Compounds of the invention may further be used with other inactivating or decontaminating agents or drugs to render inactive or weakly infective spaces, sources, surfaces or substances that have been contaminated with HCV or other viruses. For instance, allograft or xenograft tissues, blood, surgical instrument surfaces, syringes, garments, and transfusion apparatuses that pose a viral infective risk to others may be rendered virally inactive or weakly infective by use of the compounds. In another example, airborne virally-contaminated blood particles pose an infective risk. In the presence of such a risk, a compound of the invention may be dispersed as an aerosol in the contaminated space in an effective amount to inactivate or diminish the infectivity of the airborne virus by contact with the virus.

Other anti-HCV compounds or drugs which can be used in combination with the compounds of the present invention include, without limitation. HCV protease inhibitors. HCV metalloprotease inhibitors. HCV serine protease inhibitors, HCV RNA polymerase inhibitors (HCV RdRp inhibitors), HCV helicase inhibitors, interferons, interferon-alpha or pegylated interferon-alpha (INF-α), interferon-α-2β, ribavirin (e.g., Pegasys®; Copegus® (Roche)), a combination of interferon-alpha or pegylated interferon-alpha (INF-α), interferon-α-2β, ribavirin, anti-HCV monoclonal antibodies, anti-HCV humanized antibodies, anti-HCV polyclonal antibodies. IRES inhibitors, antisense compounds, anti-viral small molecules, and ribozymes, or a combination of the foregoing. The compounds of the invention can be used in combination with anti-cancer agents, anti-infective agents, and combinations thereof. The compounds of the invention may be co-administered with one or more additional antivirals, anti-HCV drugs, anti-cancer agents, or anti-infective agents. Co-administration can involve providing one or more compounds of the invention in a composition comprising one or more antivirals, anti-HCV drugs, or other drugs. Co-administration can also involve administering or providing one or more compounds of the invention, or a pharmaceutically acceptable composition comprising one or more compounds of the invention, at the same time as, at a time before, or at a time after another antiviral, anti-HCV drug, or other drug is administered or provided to a subject in need of such treatment. Administration of one or more compounds of the invention can alternate with the administration of one or more additional antivirals, anti-HCV drugs, or other drugs as described.

Some antiviral agents in development are orally bioavailable inhibitors targeting the HCV NS3/4A protease and the HCV NS5B polymerase. Illustratively and without limitation, telaprevir/VX-950 (Vertex/J&J) and Boceprevir/SCH503034 (Schering-Plough) are examples of compounds in late stage clinical testing that target the HCV NS3/4A protease. Other HCV inhibitors in clinical development include macrocyclic NS3/4A protease inhibitors, such as ITMN-191/R7227 (Intermune/Roche), BI-201335 (Boehringer Ingelheim), TMC435350 (Medivir/J&J) and MK7009 (Merck); NS5B polymerase inhibitors, such as R7128 (Pharmasset/Roche), VCH-759 (ViRochem Pharma), PF-00868554 (Pfizer), ANA-598 (Anadys); and NS5A inhibitors BMS-790052 (Bristol-Myers Squibb). Accordingly, an embodiment of the invention encompasses the administration of one or more compounds of this invention with one or more drugs, such as those above, as combination therapy, for example, to provide treatment regimens involving a wider repertoire of HCV inhibitor compounds having different and novel mechanisms of action. In an embodiment, the anti-HCV inhibitory activity of a compound of the invention is synergistic or additive with one or more of the abovementioned therapeutics or compounds, which is used in combination with the inventive compound. In an embodiment, the compound of the invention is PRO206.

The pharmaceutical preparations of the present invention may include, or be diluted into, a pharmaceutically-acceptable carrier. The term “pharmaceutically-acceptable carrier” as used herein means one or more compatible solid or liquid fillers, diluents or encapsulating substances which are suitable for administration to a human or other mammal such as non-human primate, for example, a dog, cat, horse, cow, sheep, pig, or goat.

The terms “carrier” or “vehicle” denote an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington: The Science and Practice of Pharmacy, 20th Edition. (Alfanso R. Gennaro): Lippincott Williams & Wilkins, Baltimore, Md., 2000.

Aqueous formulations may include one or more chelating agents, buffering agents, anti-oxidants and, optionally, isotonicity agents, preferably pH adjusted, for example, to between 3.0 and 3.5.

Chelating agents include, for example and without limitation, ethylenediaminetetraacetic acid (EDTA) and derivatives thereof, citric acid and derivatives thereof, niacinamide and derivatives thereof, sodium desoxycholate and derivatives thereof, and L-glutamic acid, N,N-diacetic acid and derivatives thereof.

Buffering agents include, without limitation, those selected from the group consisting of citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid, and combinations thereof.

Antioxidants include, without limitation, those selected from the group consisting of an ascorbic acid derivative, butylated hydroxy anisole, butylated hydroxy toluene, alkyl gallate, sodium meta-bisulfite, sodium bisulfite, sodium dithionite, sodium thioglycollate acid, sodium formaldehyde sulfoxylate, tocopherol and derivatives thereof, monothioglycerol, sodium sulfite, and combinations thereof.

Isotonicity agents include, without limitation, those selected from the group consisting of sodium chloride, mannitol, lactose, dextrose, glycerol, and sorbitol and combinations thereof.

Preservatives that can be used with the present compositions include, without limitation, benzyl alcohol, parabens, thimerosal, chlorobutanol and preferably benzalkonium chloride and combinations thereof. Typically, the preservative is present in a composition in a concentration of up to about 2% by weight. The exact concentration of the preservative, however, will vary depending upon the intended use and can be easily ascertained by one skilled in the art.

The compounds of the invention can be prepared in lyophilized compositions, preferably in the presence of one or more cryoprotecting agents such as trehalose, mannitol, lactose, sucrose, polyethylene glycol, and polyvinyl pyrrolidines. Cryoprotecting agents that result in a reconstitution pH of 6.0 or less are suitable. The invention therefore provides a lyophilized preparation of compounds and/or compositions of the invention. The preparation can contain a cryoprotecting agent, such as mannitol or lactose, which is preferably neutral or acidic in water.

Oral, parenteral and suppository formulations of agents are well known and commercially available. The therapeutic compounds and/or compositions of the invention can be added to such well known formulations, which can be mixed together in solution or semi-solid solution in such formulations, can be provided in a suspension within such formulations, or can be contained in particles within such formulations.

A product containing one or more therapeutic compounds of the invention and, optionally, one or more other active agents can be configured as an oral dosage. In an embodiment of the present invention, one or more HCV inhibitor compounds of the invention are orally bioavailable, or are provided as orally bioavailable products or pharmaceutically acceptable compositions. The oral dosage may be a liquid, a semisolid or a solid. The oral dosage may be configured to release the therapeutic compound of the invention before, after, or simultaneously with the other agent. The oral dosage may be configured to have the therapeutic compound of the invention and the other agents release completely in the stomach, release partially in the stomach and partially in the intestine, in the intestine, in the colon, partially in the stomach, or wholly in the colon. The oral dosage also may be configured whereby the release of the therapeutic compound of the invention is confined to the stomach or intestine while the release of the other active agent is not so confined or is confined differently from the therapeutic compound of the invention. For example, the therapeutic compound of the invention may comprise an enterically coated core or pellets contained within a pill or capsule that releases the other agent first and releases the therapeutic compound of the invention only after the therapeutic compound of the invention passes through the stomach and into the intestine. A therapeutic compound of the invention also can be in a sustained release material, whereby the therapeutic compound of the invention is released throughout the gastrointestinal tract and the other agent is released on the same or a different schedule. The same objective for a therapeutic compound of the invention can be achieved with an immediate release of the therapeutic compound of the invention, combined with an enteric coated therapeutic compound of the invention. In this instance, the therapeutic compound could be released immediately in the stomach, throughout the gastrointestinal tract, or only in the intestine.

The materials useful for achieving these different release profiles are well known to those of ordinary skill in the art. Immediate release is obtainable by conventional tablets with binders which dissolve in the stomach. Coatings which dissolve at the pH of the stomach or which dissolve at elevated temperatures will achieve the same purpose. Release only in the intestine is achieved using conventional enteric coatings such as pH sensitive coatings which dissolve in the pH environment of the intestine (but not the stomach) or coatings which dissolve over time. Release throughout the gastrointestinal tract is achieved by using sustained-release materials and/or combinations of the immediate release systems and sustained and/or delayed intentional release systems (e.g., pellets which dissolve at different pHs).

In the event that it is desirable to release the therapeutic compound of the invention first, a therapeutic compound of the invention could be coated on the surface of the controlled release formulation in any pharmaceutically acceptable carrier suitable for such coatings and for permitting the release of the therapeutic agent of the invention, such as in a temperature sensitive pharmaceutically acceptable carrier used for controlled release routinely. Other coatings which dissolve when placed in the body are well known to those of ordinary skill in the art.

A therapeutic compound of the invention also may be mixed throughout a controlled release formulation, whereby it is released before, after or simultaneously with another agent. The therapeutic compound of the invention may be free, that is, solubilized within the material of the formulation. The therapeutic compound of the invention also may be in the form of vesicles, such as wax coated micropellets dispersed throughout the material of the formulation. The coated pellets can be fashioned to immediately release the therapeutic compound of the invention based on temperature, pH, or the like. The pellets also can be configured so as to delay the release of the therapeutic compound of the invention, allowing the other agent a period of time to act before the therapeutic compound of the invention exerts its effects. The therapeutic compound of the invention pellets also can be configured to release the therapeutic compound of the invention in virtually any sustained release pattern, including patterns exhibiting first order release kinetics or sigmoidal order release kinetics using materials of the prior art and well known to those of ordinary skill in the art.

A therapeutic compound of the invention also can be contained within a core within the controlled release formulation. The core may have any one or any combination of the properties described above in connection with the pellets. The therapeutic agent of the invention may be, for example, in a core coated with a material, dispersed throughout a material, coated onto a material or adsorbed into or throughout a material. It should be understood that the pellets or core may be of virtually any type. They may be drug coated with a release material, drug interspersed throughout material, drug adsorbed into a material, and so on. The material may be erodible or nonerodible.

A therapeutic compound of the invention also may be mixed throughout a controlled release formulation, whereby it is released before, after or simultaneously with another agent. The therapeutic compound of the invention may be free, that is, solubilized within the material of the formulation. The therapeutic compound of the invention also may be in the form of vesicles, such as wax coated micropellets dispersed throughout the material of the formulation. The coated pellets can be fashioned to immediately release the therapeutic compound of the invention based on temperature, pH, or the like. The pellets also can be configured so as to delay the release of the therapeutic compound of the invention, allowing the other agent a period of time to act before the therapeutic compound of the invention exerts its effects. The therapeutic compound of the invention pellets also can be configured to release the therapeutic compound of the invention in virtually any sustained release pattern, including patterns exhibiting first order release kinetics or sigmoidal order release kinetics using materials of the prior art and well known to those of ordinary skill in the art.

A therapeutic compound of the invention also can be contained within a core within the controlled release formulation. The core may have any one or any combination of the properties described above in connection with the pellets. The therapeutic agent of the invention may be, for example, in a core coated with a material, dispersed throughout a material, coated onto a material or adsorbed into or throughout a material. It should be understood that the pellets or core may be of virtually any type. They may be drug coated with a release material, drug interspersed throughout material, drug adsorbed into a material, and so on. The material may be erodible or nonerodible.

A therapeutic compound of the invention may be provided in particles. Particles as used herein means nano- or microparticles (or in some instances larger) which can consist in whole or in part of a compound of the invention or other agents as described herein. The particles may contain the therapeutic compounds in a core surrounded by a coating, including, but not limited to, an enteric coating. Such compounds also may be dispersed throughout the particles. These compounds also may be adsorbed into the particles. The particles may be of any order release kinetics, including zero order release, first order release, second order release, delayed release, sustained release, immediate release, and any combination thereof, etc. The particle may include, in addition to the therapeutic compound, any of those materials routinely used in the art of pharmacy and medicine, including, but not limited to, erodible, nonerodible, biodegradable, or nonbiodegradable material or combinations thereof. The particles may be microcapsules which contain the antiviral compound in a solution or in a semi-solid state. The particles may be of virtually any shape.

Both non-biodegradable and biodegradable polymeric materials can be used in the manufacture of particles for delivering the therapeutic compounds of the invention. Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic compounds of the invention may be contained in controlled release systems. The term “controlled release” is intended to refer to any drug-containing formulation in which the manner and profile of drug release from the formulation are controlled. This refers to immediate as well as nonimmediate release formulations, with nonimmediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.” These formulations may be for any mode of administration.

Delivery systems specific for the gastrointestinal tract are roughly divided into three types: the first is a delayed release system designed to release a drug in response to, for example, a change in pH; the second is a timed-release system designed to release a drug after a predetermined time; and the third is a microflora enzyme system making use of the abundant enterobacteria in the lower part of the gastrointestinal tract (e.g., in a colonic site-directed release formulation).

An example of a delayed release system is one that uses, for example, an acrylic or cellulosic coating material and dissolves on pH change. Because of ease of preparation, many reports on such “enteric coatings” have been made. In general, an enteric coating is one which passes through the stomach without releasing substantial amounts of drug in the stomach (i.e., less than 10% release, 5% release and even 1% release in the stomach) and sufficiently disintegrating in the intestinal tract (by contact with approximately neutral or alkaline intestine juices) to allow the transport (active or passive) of the active agent through the walls of the intestinal tract.

Various in vitro tests for determining whether or not a coating is classified as an enteric coating have been published in the pharmacopoeia of various countries. A coating which remains intact for at least 2 hours, in contact with artificial gastric juices such as HCl of pH 1 at 36 to 38° C. and thereafter disintegrates within 30 minutes in artificial intestinal juices such as a KH₂PO₄ buffered solution of pH 6.8 is one example. One such well known system is EUDRAGIT® material, commercially available and reported on by Behringer, Manchester University, Saale Co., and the like. Enteric coatings are discussed further, below.

The enteric coating is typically, although not necessarily, a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl methacrylate (e.g., those copolymers sold under the trade name EUDRAGIT®); vinyl polymers and copolymers such as polyvinyl acetate, polyvinylacetate phthalate, vinylacetate crotonic acid copolymer, and ethylene-vinyl acetate copolymers; and shellac (purified lac). Combinations of different coating materials may also be used. Well known enteric coating material for use herein are those acrylic acid polymers and copolymers available under the trade name EUDRAGIT® from Rohm Pharma (Germany). The EUDRAGIT® series E, L, S, RL, RS and NE copolymers are available as solubilized in organic solvent, as an aqueous dispersion, or as a dry powder. The EUDRAGIT® series RL, NE, and RS copolymers are insoluble in the gastrointestinal tract but are permeable and are used primarily for extended release. The EUDRAGIT® series E copolymers dissolve in the stomach. The EUDRAGIT® series L, L-30D and S copolymers are insoluble in stomach and dissolve in the intestine, and are thus most preferred herein.

A particular methacrylic copolymer is EUDRAGIT® L, particularly L-30D and EUDRAGIT® L 100-55. In EUDRAGIT® L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particular methacrylic acid polymer is EUDRAGIT® S, which differs from EUDRAGIT® L-30D in that the ratio of free carboxyl groups to ester groups is approximately 1:2. EUDRAGIT® S is insoluble at pH below 5.5, but unlike EUDRAGIT® L-30D, is poorly soluble in gastrointestinal fluids having a pH in the range of 5.5 to 7.0, such as in the small intestine. This copolymer is soluble at pH 7.0 and above, i.e., the pH generally found in the colon. EUDRAGIT® S can be used alone as a coating to provide drug delivery in the large intestine. Alternatively, EUDRAGIT® S, being poorly soluble in intestinal fluids below pH 7, can be used in combination with EUDRAGIT® L-30D, soluble in intestinal fluids above pH 5.5, in order to provide a delayed release composition which can be formulated to deliver the active agent to various segments of the intestinal tract. The more EUDRAGIT L-30D used, the more proximal release and delivery begins, and the more EUDRAGIT® S used, the more distal release and delivery begins. It will be appreciated by those skilled in the art that both EUDRAGIT® L-30D and EUDRAGIT® S can be replaced with other pharmaceutically acceptable polymers having similar pH solubility characteristics. In certain embodiments of the invention, the preferred enteric coating is ACRYL-EZE™ (methacrylic acid co-polymer type C; Colorcon, West Point, Pa.).

The enteric coating provides for controlled release of the active agent, such that drug release can be accomplished at some generally predictable location. The enteric coating also prevents exposure of the therapeutic and/or agent and carrier to the epithelial and mucosal tissue of the buccal cavity, pharynx, esophagus, and stomach, and to the enzymes associated with these tissues. The enteric coating therefore helps to protect the active agent, carrier and a patient's internal tissue from any adverse event prior to drug release at the desired site of delivery. Furthermore, the coated material of the present invention allows optimization of drug absorption, active agent protection, and safety. Multiple enteric coatings targeted to release the active agent at various regions in the gastrointestinal tract would enable even more effective and sustained improved delivery throughout the gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex® 2), triacetin (glyceryl triacetate), acetyl triethyl citrate (Citroflec® A2), Carbowax™ 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to particles of the therapeutic and/or agent(s), tablets of the therapeutic and/or agent(s), capsules containing the therapeutic agent(s) and the like, using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6th Ed. (Media, Pa.: Williams & Wilkins, 1995). The coating thickness, as noted above, must be sufficient to ensure that the oral dosage form remains intact until the desired site of topical delivery in the lower intestinal tract is reached.

In another embodiment, drug dosage forms are provided that comprise an enterically coated, osmotically activated device housing a formulation of the invention. In this embodiment, the drug-containing formulation is encapsulated in a semipermeable membrane or barrier containing a small orifice. As known in the art with respect to so-called “osmotic pump” drug delivery devices, the semipermeable membrane allows passage of water, but not drug, in either direction. Therefore, when the device is exposed to aqueous fluids, water will flow into the device due to the osmotic pressure differential between the interior and exterior of the device. As water flows into the device, the drug-containing formulation in the interior will be “pumped” out through the orifice. The rate of drug release will be equivalent to the inflow rate of water times the drug concentration. The rate of water influx and drug efflux can be controlled by the composition and size of the orifice of the device. Suitable materials for the semipermeable membrane include, but are not limited to, polyvinyl alcohol, polyvinyl chloride, semipermeable polyethylene glycols, semipermeable polyurethanes, semipermeable polyamides, semipermeable sulfonated polystyrenes and polystyrene derivatives; semipermeable poly(sodium styrenesulfonate), semipermeable poly(vinylbenzyltrimethylammonium chloride), and cellulosic polymers such as cellulose acetate, cellulose diacetate, cellulose triacetate, cellulose propionate, cellulose acetate propionate, cellulose acetate butyrate, cellulose trivalerate, cellulose trilmate, cellulose tripalmitate, cellulose trioctanoate, cellulose tripropionate, cellulose disuccinate, cellulose dipalmitate, cellulose dicylate, cellulose acetate succinate, cellulose propionate succinate, cellulose acetate octanoate, cellulose valerate palmitate, cellulose acetate heptanate, cellulose acetaldehyde dimethyl acetal, cellulose acetate ethylcarbamate, cellulose acetate methylcarbamate, cellulose dimethylaminoacetate and ethylcellulose.

In another embodiment, drug dosage forms are provided that comprise a sustained release coated device housing a formulation of the invention. In this embodiment, the drug-containing formulation is encapsulated in a sustained release membrane or film. The membrane may be semipermeable, as described above. A semipermeable membrane allows for the passage of water inside the coated device to dissolve the drug. The dissolved drug solution diffuses out through the semipermeable membrane. The rate of drug release depends upon the thickness of the coated film and the release of drug can begin in any part of the GI tract. Suitable membrane materials for such a membrane include ethylcellulose.

In another embodiment, drug dosage forms are provided that comprise a sustained release device housing a formulation of the invention. In this embodiment, the drug-containing formulation is uniformly mixed with a sustained release polymer. These sustained release polymers are high molecular weight water-soluble polymers, which when in contact with water, swell and create channels for water to diffuse inside and dissolve the drug. As the polymers swell and dissolve in water, more of drug is exposed to water for dissolution. Such a system is generally referred to as sustained release matrix. Suitable materials for such a device include hydropropyl methylcellulose, hydroxypropyl cellulose, hydroxyethyl cellulose and methyl cellulose.

In another embodiment, drug dosage forms are provided that comprise an enteric coated device housing a sustained release formulation of the invention. In this embodiment, the drug containing product described above is coated with an enteric polymer. Such a device would not release any drug in the stomach and when the device reaches the intestine, the enteric polymer is first dissolved and only then would the drug release begin. The drug release would take place in a sustained release fashion.

Enterically coated, osmotically activated devices can be manufactured using conventional materials, methods and equipment. For example, osmotically activated devices may be made by first encapsulating, in a pharmaceutically acceptable soft capsule, a liquid or semi-solid formulation of the compounds of the invention as described previously. This interior capsule is then coated with a semipermeable membrane composition (comprising, for example, cellulose acetate and polyethylene glycol 4000 in a suitable solvent such as a methylene chloride-methanol admixture), for example using an air suspension machine, until a sufficiently thick laminate is formed, e.g., around 0.05 mm. The semipermeable laminated capsule is then dried using conventional techniques. Then, an orifice having a desired diameter (e.g., about 0.99 mm) is provided through the semipermeable laminated capsule wall, using, for example, mechanical drilling, laser drilling, mechanical rupturing, or erosion of an erodible element such as a gelatin plug. The osmotically activated device may then be enterically coated as previously described. For osmotically activated devices containing a solid carrier rather than a liquid or semi-solid carrier, the interior capsule is optional; that is, the semipermeable membrane may be formed directly around the carrier-drug composition. However, preferred carriers for use in the drug-containing formulation of the osmotically activated device are solutions, suspensions, liquids, immiscible liquids, emulsions, sols, colloids, and oils. Particularly preferred carriers include, but are not limited to, those used for enterically coated capsules containing liquid or semisolid drug formulations.

Cellulose coatings include those of cellulose acetate phthalate and trimellitate; methacrylic acid copolymers, e.g. copolymers derived from methylacrylic acid and esters thereof, containing at least 40% methylacrylic acid; and especially hydroxypropyl methylcellulose phthalate. Methylacrylates include those of molecular weight above 100,000 daltons based on, e.g. methylacrylate and methyl or ethyl methylacrylate in a ratio of about 1:1. Typical products include Endragit L, e.g. L 100-55, marketed by Rohm GmbH, Darmstadt, Germany. Typical cellulose acetate phthalates have an acetyl content of 17-26% and a phthalate content of from 30-40% with a viscosity of ca. 45-90 cP. Typical cellulose acetate trimellitates have an acetyl content of 17-26%, a trimellityl content from 25-35% with a viscosity of ca. 15-20 cS. An example of a cellulose acetate trimellitate is the marketed product CAT (Eastman Kodak Company, USA). Hydroxypropyl methylcellulose phthalates typically have a molecular weight of from 20,000 to 130,000 daltons, a hydroxypropyl content of from 5 to 10%, a methoxy content of from 18 to 24% and a phthalyl content from 21 to 35%. An example of a cellulose acetate phthalate is the marketed product CAP (Eastman Kodak, Rochester N.Y., USA). Examples of hydroxypropyl methylcellulose phthalates are the marketed products having a hydroxypropyl content of from 6-10%, a methoxy content of from 20-24%, a phthalyl content of from 21-27%, a molecular weight of about 84,000 daltons, sold under the trademark HP50 and available from Shin-Etsu Chemical Co. Ltd., Tokyo, Japan, and having a hydroxypropyl content, a methoxyl content, and a phthalyl content of 5-9%, 18-22% and 27-35%, respectively, and a molecular weight of 78,000 daltons, known under the trademark HP55 and available from the same supplier.

A timed release system is represented by Time Erosion System (TES) by Fujisawa Pharmaceutical Co., Ltd. and Pulsincap by R. P. Scherer. According to these systems, the site of drug release is decided by the time of transit of a preparation in the gastrointestinal tract. Since the transit of a preparation in the gastrointestinal tract is largely influenced by the gastric emptying time, some time release systems are also enterically coated.

Systems making use of the enterobacteria can be classified into those utilizing degradation of azoaromatic polymers by an azo reductase produced from enterobacteria as reported by the group of Ohio University (M. Saffran, et al., Science, Vol. 233: 1081 (1986)) and the group of Utah University (J. Kopecek, et al., Pharmaceutical Research, 9(12), 1540-1545 (1992)); and those utilizing degradation of polysaccharides by beta-galactosidase of enterobacteria as reported by the group of Hebrew University (unexamined published Japanese patent application No. 5-50863 based on a PCT application) and the group of Freiberg University (K. H. Bauer et al., Pharmaceutical Research, 10(10), S218 (1993)). In addition, the system using chitosan degradable by chitosanase by Teikoku Seiyaku K. K. (unexamined published Japanese patent application No. 4-217924 and unexamined published Japanese patent application No. 4-225922) is also included.

A therapeutic compound of the invention may be provided in particles. Particles as used herein means nano- or microparticles (or in some instances larger) which can consist in whole or in part of a compound of the invention or other agents as described herein. The particles may contain the therapeutic compounds in a core surrounded by a coating, including, but not limited to, an enteric coating. Such compounds also may be dispersed throughout the particles. These compounds also may be adsorbed into the particles. The particles may be of any order release kinetics, including zero order release, first order release, second order release, delayed release, sustained release, immediate release, and any combination thereof, etc. The particle may include, in addition to the therapeutic compound, any of those materials routinely used in the art of pharmacy and medicine, including, but not limited to, erodible, nonerodible, biodegradable, or nonbiodegradable material or combinations thereof. The particles may be microcapsules which contain the antiviral compound in a solution or in a semi-solid state. The particles may be of virtually any shape.

Both non-biodegradable and biodegradable polymeric materials can be used in the manufacture of particles for delivering the therapeutic compounds of the invention. Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In another embodiment, drug dosage forms are provided that comprise a sustained release coated device housing a formulation of the invention. In this embodiment, the drug-containing formulation is encapsulated in a sustained release membrane or film. The membrane may be semipermeable, as described above. A semipermeable membrane allows for the passage of water inside the coated device to dissolve the drug. The dissolved drug solution diffuses out through the semipermeable membrane. The rate of drug release depends upon the thickness of the coated film and the release of drug can begin in any part of the GI tract. Suitable membrane materials for such a membrane include ethylcellulose.

In another embodiment, drug dosage forms are provided that comprise a sustained release device housing a formulation of the invention. In this embodiment, the drug-containing formulation is uniformly mixed with a sustained release polymer. These sustained release polymers are high molecular weight water-soluble polymers, which when in contact with water, swell and create channels for water to diffuse inside and dissolve the drug. As the polymers swell and dissolve in water, more of drug is exposed to water for dissolution. Such a system is generally referred to as sustained release matrix. Suitable materials for such a device include hydropropyl methylcellulose, hydroxypropyl cellulose, hydroxyethyl cellulose and methyl cellulose.

In another embodiment, drug dosage forms are provided that comprise an enteric coated device housing a sustained release formulation of the invention. In this embodiment, the drug containing product described above is coated with an enteric polymer. Such a device would not release any drug in the stomach and when the device reaches the intestine, the enteric polymer is first dissolved and only then would the drug release begin. The drug release would take place in a sustained release fashion.

The therapeutic compounds may be provided in capsules, coated or not. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals. A product containing one or more therapeutic compounds of the invention can be configured as a suppository. The therapeutic compound of the invention can be placed anywhere within or on the suppository to favorably affect the relative release of the therapeutic compound. The nature of the release can be zero order, first order, or sigmoidal, as desired.

Suppositories are solid dosage forms of medicine intended for administration via the rectum. Suppositories are compounded so as to melt, soften, or dissolve in the body cavity (around 98.6° F.) thereby releasing the medication contained therein. Suppository bases should be stable, nonirritating, chemically inert, and physiologically inert. Many commercially available suppositories contain oily or fatty base materials, such as cocoa butter, coconut oil, palm kernel oil, and palm oil, which often melt or deform at room temperature necessitating cool storage or other storage limitations. U.S. Pat. No. 4,837,214 to Tanaka et al. describes a suppository base comprised of 80 to 99 percent by weight of a lauric-type fat having a hydroxyl value of 20 or smaller and containing glycerides of fatty acids having 8 to 18 carbon atoms combined with 1 to 20 percent by weight diglycerides of fatty acids (which erucic acid is an example of). The shelf life of these type of suppositories is limited due to degradation. Other suppository bases contain alcohols, surfactants, and the like which raise the melting temperature but also can lead to poor absorption of the medicine and side effects due to irritation of the local mucous membranes (see for example, U.S. Pat. No. 6,099,853 to Hartelendy et al., U.S. Pat. No. 4,999,342 to Ahmad et al., and U.S. Pat. No. 4,765,978 to Abidi et al.).

The base used in the pharmaceutical suppository composition of this invention includes, in general, oils and fats comprising triglycerides as main components such as cacao butter, palm fat, palm kernel oil, coconut oil, fractionated coconut oil, lard and WITEPSOL®, waxes such as lanolin and reduced lanolin; hydrocarbons such as VASELINE®, squalene, squalane and liquid paraffin; long to medium chain fatty acids such as caprylic acid, lauric acid, stearic acid and oleic acid; higher alcohols such as lauryl alcohol, cetanol and stearyl alcohol; fatty acid esters such as butyl stearate and dilauryl malonate; medium to long chain carboxylic acid esters of glycerin such as triolein and tristearin; glycerin-substituted carboxylic acid esters such as glycerin acetoacetate; and polyethylene glycols and its derivatives such as macrogols and cetomacrogol. They may be used either singly or in combination of two or more. If desired, the composition of this invention may further include a surface-active agent, a coloring agent, etc., which are ordinarily used in suppositories.

The pharmaceutical composition of this invention may be prepared by uniformly mixing predetermined amounts of the active ingredient, the absorption aid and optionally the base, etc. in a stirrer or a grinding mill, if required at an elevated temperature. The resulting composition may be formed into a suppository in unit dosage form by, for example, casting the mixture in a mold, or by forming it into a gelatin capsule using a capsule filling machine.

The compositions according to the present invention also can be administered as a nasal spray, nasal drop, solution, suspension, gel, ointment, cream or powder. The administration of a composition can also include using a nasal tampon or a nasal sponge containing a composition of the present invention.

The nasal delivery systems that can be used with the present invention can take various forms including aqueous preparations, non-aqueous preparations and combinations thereof. Aqueous preparations include, for example, aqueous gels, aqueous suspensions, aqueous liposomal dispersions, aqueous emulsions, aqueous microemulsions and combinations thereof. Non-aqueous preparations include, for example, non-aqueous gels, non-aqueous suspensions, non-aqueous liposomal dispersions, non-aqueous emulsions, non-aqueous microemulsions and combinations thereof. The various forms of the nasal delivery systems can include a buffer to maintain pH, a pharmaceutically acceptable thickening agent and a humectant. The pH of the buffer can be selected to optimize the absorption of the therapeutic agent(s) across the nasal mucosa.

With respect to the non-aqueous nasal formulations, suitable forms of buffering agents can be selected such that when the formulation is delivered into the nasal cavity of a mammal, selected pH ranges are achieved therein upon contact with, e.g., a nasal mucosa. In the present invention, the pH of the compositions may be maintained from about 2.0 to about 6.0. It is desirable that the pH of the compositions is one which does not cause significant irritation to the nasal mucosa of a recipient upon administration.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

The compositions of the present invention can also include a humectant to reduce or prevent drying of the mucus membrane and to prevent irritation thereof. Suitable humectants that can be used in the present invention include sorbitol, mineral oil, vegetable oil and glycerol; soothing agents; membrane conditioners; sweeteners; and combinations thereof. The concentration of the humectant in the present compositions will vary depending upon the agent selected.

One or more therapeutic and/or agents may be incorporated into the nasal delivery system or any other delivery system described herein.

A composition formulated for topical administration may be liquid or semi-solid (including, for example, a gel, lotion, emulsion, cream, ointment, spray or aerosol) or may be provided in combination with a “finite” carrier, for example, a non-spreading material that retains its form, including, for example, a patch, bioadhesive, dressing or bandage. It may be aqueous or non-aqueous; it may be formulated as a solution, emulsion, dispersion, a suspension or any other mixture.

Important modes of administration include topical application to the skin, eyes or mucosa. Thus, typical vehicles are those suitable for pharmaceutical or cosmetic application to body surfaces. The compositions provided herein may be applied topically or locally to various areas in the body of a patient. As noted above, topical application is intended to refer to application to the tissue of an accessible body surface, such as, for example, the skin (the outer integument or covering) and the mucosa (the mucous-producing, secreting and/or containing surfaces). Exemplary mucosal surfaces include the mucosal surfaces of the eyes, mouth (such as the lips, tongue, gums, cheeks, sublingual and roof of the mouth), larynx, esophagus, bronchial, nasal passages, vagina and rectum/anus; in some embodiments, preferably the mouth, larynx, esophagus, vagina and rectum/anus; in other embodiments, preferably the eyes, larynx, esophagus, bronchial, nasal passages, and vagina and rectum/anus. As noted above, local application herein refers to application to a discrete internal area of the body, such as, for example, a joint, soft tissue area (such as muscle, tendon, ligaments, intraocular or other fleshy internal areas), or other internal area of the body. Thus, as used herein, local application refers to applications to discrete areas of the body.

With respect to topical and/or local administration of the present compositions, desirable efficacy may involve, for example, penetration of therapeutic agent(s) of the invention into the skin and/or tissue to substantially reach systemic circulation or a peripheral or central locus.

Also in certain embodiments, including embodiments that involve aqueous vehicles, the compositions may also contain a glycol, that is, a compound containing two or more hydroxy groups. A glycol which may be particularly useful for use in the compositions is propylene glycol. The glycol may be included in the compositions in a concentration of from greater than 0 to about 5 wt. %, based on the total weight of the composition.

For local internal administration, such as intra-articular administration, the compositions are preferably formulated as a solution or a suspension in an aqueous-based medium, such as isotonically buffered saline or are combined with a biocompatible support or bioadhesive intended for internal administration. Lotions, which, for example, may be in the form of a suspension, dispersion or emulsion, contain an effective concentration of one or more of the compounds. The effective concentration is preferably to deliver an effective amount. For example, the compound of the present invention may find use at a concentration of between about 0.1-50% [by weight] or more of one or more of the compounds provided herein.

The lotions may contain, for example, [by weight] from 1% to 50% of an emollient and the balance water, a suitable buffer, and other agents as described above. Any emollients known to those of skill in the art as suitable for application to human skin may be used. These include, but are not limited to, the following: (a) Hydrocarbon oils and waxes, including mineral oil, petrolatum, paraffin, ceresin, ozokerite, microcrystalline wax, polyethylene, and perhydrosqualene. b) Silicone oils, including dimethylpolysiloxanes, methylphenylpolysiloxanes, water-soluble and alcohol-soluble silicone-glycol copolymers. (c) Triglyceride fats and oils, including those derived from vegetable, animal and marine sources. Examples include, but are not limited to, castor oil, safflower oil, cotton seed oil, corn oil, olive oil, cod liver oil, almond oil, avocado oil, palm oil, sesame oil, and soybean oil. (d) Acetoglyceride esters, such as acetylated monoglycerides. (e) Ethoxylated glycerides, such as ethoxylated glyceryl monostearate. (f) Alkyl esters of fatty acids having 10 to 20 carbon atoms. Methyl, isopropyl and butyl esters of fatty acids are useful herein. Examples include, but are not limited to, hexyl laurate, isohexyl laurate, isohexyl palmitate, isopropyl palmitate, isopropyl myristate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate. (g) Alkenyl esters of fatty acids having 10 to 20 carbon atoms. Examples thereof include, but are not limited to, oleyl myristate, oleyl stearate, and oleyl oleate. (h) Fatty acids having 9 to 22 carbon atoms. Suitable examples include, but are not limited to, pelargonic, lauric, myristic, palmitic, stearic, isostearic, hydroxystearic, oleic, linoleic, ricinoleic, arachidonic, behenic, and erucic acids. (i) Fatty alcohols having 10 to 22 carbon atoms, such as, but not limited to, lauryl, myristyl, cetyl, hexadecyl, stearyl, isostearyl, hydroxystearyl, oleyl, ricinoleyl, behenyl, erucyl, and 2-octyl dodecyl alcohols. (j) Fatty alcohol ethers, including, but not limited to ethoxylated fatty alcohols of 10 to 20 carbon atoms, such as, but are not limited to, the lauryl, cetyl, stearyl, isostearyl, oleyl, and cholesterol alcohols having attached thereto from 1 to 50 ethylene oxide groups or 1 to 50 propylene oxide groups or mixtures thereof. (k) Ether-esters, such as fatty acid esters of ethoxylated fatty alcohols. (l) Lanolin and derivatives, including, but not limited to, lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases. (m) polyhydric alcohols and polyether derivatives, including, but not limited to, propylene glycol, dipropylene glycol, polypropylene glycol [M.W. 2000-4000], polyoxyethylene polyoxypropylene glycols, polyoxypropylene polyoxyethylene glycols, glycerol, ethoxylated glycerol, propoxylated glycerol, sorbitol, ethoxylated sorbitol, hydroxypropyl sorbitol, polyethylene glycol [M.W. 200-6000], methoxy polyethylene glycols 350, 550, 750, 2000, 5000, poly(ethylene oxide) homopolymers [M.W. 100,000-5,000,000], polyalkylene glycols and derivatives, hexylene glycol (2-methyl-2,4-pentanediol), 1,3-butylene glycol, 1,2,6,-hexanetriol, ethohexadiol USP (2-ethyl-1,3-hexanediol), C₁₅-C₁₈ vicinal glycol and polyoxypropylene derivatives of trimethylolpropane. (n) polyhydric alcohol esters, including, but not limited to, ethylene glycol mono- and di-fatty acid esters, diethylene glycol mono- and di-fatty acid esters, polyethylene glycol [M.W. 200-6000], mono- and di-fatty esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters. (o) Wax esters, including, but not limited to, beeswax, spermaceti, myristyl myristate, and stearyl stearate and beeswax derivatives, including, but not limited to, polyoxyethylene sorbitol beeswax, which are reaction products of beeswax with ethoxylated sorbitol of varying ethylene oxide content that form a mixture of ether-esters. (p) Vegetable waxes, including, but not limited to, carnauba and candelilla waxes. (q) phospholipids, such as lecithin and derivatives. (r) Sterols, including, but not limited to, cholesterol and cholesterol fatty acid esters. (s) Amides, such as fatty acid amides, ethoxylated fatty acid amides, and solid fatty acid alkanolamides.

The lotions further preferably contain [by weight] from 1% to 10%, more preferably from 2% to 5%, of an emulsifier. The emulsifiers can be nonionic, anionic or cationic. Examples of satisfactory nonionic emulsifiers include, but are not limited to, fatty alcohols having 10 to 20 carbon atoms, fatty alcohols having 10 to 20 carbon atoms condensed with 2 to 20 moles of ethylene oxide or propylene oxide, alkyl phenols with 6 to 12 carbon atoms in the alkyl chain condensed with 2 to 20 moles of ethylene oxide, mono- and di-fatty acid esters of ethylene oxide, mono- and di-fatty acid esters of ethylene glycol where the fatty acid moiety contains from 10 to 20 carbon atoms, diethylene glycol, polyethylene glycols of molecular weight 200 to 6000, propylene glycols of molecular weight 200 to 3000, glycerol, sorbitol, sorbitan, polyoxyethylene sorbitol, polyoxyethylene sorbitan and hydrophilic wax esters. Suitable anionic emulsifiers include, but are not limited to, the fatty acid soaps, e.g., sodium, potassium and triethanolamine soaps, where the fatty acid moiety contains from 10 to 20 carbon atoms. Other suitable anionic emulsifiers include, but are not limited to, the alkali metal, ammonium or substituted ammonium alkyl sulfates, alkyl arylsulfonates, and alkyl ethoxy ether sulfonates having 10 to 30 carbon atoms in the alkyl moiety. The alkyl ethoxy ether sulfonates contain from 1 to 50 ethylene oxide units. Among satisfactory cationic emulsifiers are quaternary ammonium, morpholinium and pyridinium compounds. Certain of the emollients described in preceding paragraphs also have emulsifying properties. When a lotion is formulated containing such an emollient, an additional emulsifier is not needed, though it can be included in the composition.

The balance of the lotion is water or a C₂ or C₃ alcohol, or a mixture of water and the alcohol. The lotions are formulated by simply admixing all of the components together. Preferably the compound, is dissolved, suspended or otherwise uniformly dispersed in the mixture.

Other conventional components of such lotions may be included. One such additive is a thickening agent at a level from 1% to 10% by weight of the composition. Examples of suitable thickening agents include, but are not limited to: cross-linked carboxypolymethylene polymers, ethyl cellulose, polyethylene glycols, gum tragacanth, gum kharaya, xanthan gums and bentonite, hydroxyethyl cellulose, and hydroxypropyl cellulose.

Creams can be formulated to contain a concentration effective to deliver an effective amount of therapeutic agent(s) of the invention to the treated tissue, typically at between about 0.1%, preferably at greater than 1% up to and greater than 50%, preferably between about 3% and 50%, more preferably between about 5% and 15% therapeutic agent(s) of the invention. The creams also contain from 5% to 50%, preferably from 10% to 25%, of an emollient and the remainder is water or other suitable non-toxic carrier, such as an isotonic buffer. The emollients, as described above for the lotions, can also be used in the cream compositions. The cream may also contain a suitable emulsifier, as described above. The emulsifier is included in the composition at a level from 3% to 50%, preferably from 5% to 20%.

These compositions that are formulated as solutions or suspensions may be applied to the skin, or, may be formulated as an aerosol or foam and applied to the skin as a spray-on. The aerosol compositions typically contain [by weight] from 25% to 80%, preferably from 30% to 50%, of a suitable propellant. Examples of such propellants are the chlorinated, fluorinated and chlorofluorinated lower molecular weight hydrocarbons. Nitrous oxide, carbon dioxide, butane, and propane are also used as propellant gases. These propellants are used as understood in the art in a quantity and under a pressure suitable to expel the contents of the container.

Suitably prepared solutions and suspensions may also be topically applied to the eyes and mucosa. Solutions, particularly those intended for ophthalmic use, may be formulated as 0.01%-10% isotonic solutions, pH about 5-7, with appropriate salts, and preferably containing one or more of the compounds herein at a concentration of about 0.1%, preferably greater than 1%, up to 50% or more. Suitable ophthalmic solutions are known [see, e.g., U.S. Pat. No. 5,116,868, which describes typical compositions of ophthalmic irrigation solutions and solutions for topical application]. Such solutions, which have a pH adjusted to about 7.4, contain, for example, 90-100 mM sodium chloride, 4-6 mM dibasic potassium phosphate, 4-6 mM dibasic sodium phosphate, 8-12 mM sodium citrate, 0.5-1.5 mM magnesium chloride, 1.5-2.5 mM calcium chloride, 15-25 mM sodium acetate, 10-20 mM D,L.-sodium β-hydroxybutyrate and 5-5.5 mM glucose.

Gel compositions can be formulated by simply admixing a suitable thickening agent to the previously described solution or suspension compositions. Examples of suitable thickening agents have been previously described with respect to the lotions.

The gelled compositions contain an effective amount of therapeutic agent(s) of the invention, typically at a concentration of between about 0.1-50% by weight or more of one or more of the compounds provided herein.; from 5% to 75%, preferably from 10% to 50%, of an organic solvent as previously described; from 0.5% to 20%, preferably from 1% to 10% of the thickening agent; the balance being water or other aqueous or non-aqueous carrier, such as, for example, an organic liquid, or a mixture of carriers.

The dosing regimens, as well as the timing for administering the compounds and/or compositions of the present invention are able to be determined by the skilled practitioner in the art. Illustratively and without limitation, a compound or composition of the invention may be administered to a subject at least once per day, daily, every other day, every 6 to 8 days, weekly, bi-weekly, monthly, or bi-monthly.

The formulations can be designed and provided to create steady state plasma levels. Steady state plasma concentrations can be measured using HPLC techniques, as are known to those of skill in the art. Steady state is achieved when the rate of drug availability is equal to the rate of drug elimination from the circulation. In typical therapeutic and/or settings, the therapeutic agent(s) of the invention will be administered to patients either on a periodic dosing regimen or with a constant infusion regimen. The concentration of drug in the plasma will tend to rise immediately after the onset of administration and will tend to fall over time as the drug is eliminated from the circulation by means of distribution into cells and tissues, by metabolism, or by excretion. Steady state will be obtained when the mean drug concentration remains constant over time. In the case of intermittent dosing, the pattern of the drug concentration cycle is repeated identically in each interval between doses with the mean concentration remaining constant. In the case of constant infusion, the mean drug concentration will remain constant with very little oscillation. The achievement of steady state is determined by means of measuring the concentration of drug in plasma over at least one cycle of dosing such that one can verify that the cycle is being repeated identically from dose to dose. Typically, in an intermittent dosing regimen, maintenance of steady state can be verified by determining drug concentrations at the consecutive troughs of a cycle, just prior to administration of another dose. In a constant infusion regimen where oscillation in the concentration is low, steady state can be verified by any two consecutive measurements of drug concentration.

HCV is the most frequent indication for liver transplant in the United States and in Europe. By the year 2020, the proportion of untreated HCV patients developing cirrhosis is expected to increase by 30%, the number of cirrhotic patients with HCV to increase by 100% and the number of HCV cirrhotic patients developing hepatocellular carcinoma by 80% (Schiano T D, Martin P. 2006, “Management of HCV Infection and Liver Transplantation”, Int J Med Sci., 3:79-83; Davis G L, 2003, “Projecting future complications of chronic hepatitis C in the United States”, Liver Transpl., 9:331-8). With the anticipated increase in patients requiring liver transplants for HCV related liver disease, the development and use of effective strategies and treatments to reduce liver graft failure due to HCV recurrence is essential.

A major challenge facing liver transplant recipients and their physicians is recurrence of HCV infection following otherwise technically successful liver transplantation. Recurrent viral infection leads to diminished graft and patient survival. There is a need for therapeutics to treat and/or prevent HCV recurrence in post-transplant patients. The recurrence of HCV is typically determined by serial liver biopsies with the decision to intervene with traditional antiviral therapy based on local philosophy and expertise. (Schiano T D, Martin P. 2006, Int J Med Sci., 3:79-83). Treating hepatitis C in the liver transplant patient population has a number of major challenges, including diminished patient tolerance for side-effects as well as managing the patient's immune suppression. A treatment goal is to achieve sustained viral responses with the potential to reduce the impact of recurrent hepatitis on the graft. Unfortunately, without appropriate or optimal intervention and treatment, recurrent FICV infection is likely to remain the most frequent form of recurrent disease in liver transplant programs for the foreseeable future.

Treatment with HCV-fighting therapeutics or medications may help prevent a recurrence of infection or treat recurrent illness that develops after a liver transplant. Thus, in accordance with the present invention, one or more compounds of the invention can be used alone or in combination with one or more other anti-HCV drugs and therapeutics, such as a combination of pegylated interferon alpha-2b and ribavirin, and along with hematopoietic growth factors, as necessary, to maintain blood counts, to treat a transplant patient prior to liver transplant, particularly when the patient has an undetectable or low viral load at the time of transplant, which is associated with less severe HCV recurrence. According to this aspect of the invention, by pretreating a patient with one or more compounds of the present invention, with or without other antiviral therapeutics or anti-HCV inhibitors, the patient can be cleared of HCV prior to liver transplantation to minimize the risk associated with recurrence following transplantation.

Prophylactic and preemptive antiviral therapy using one or more compounds of the invention, alone or in combination with other HCV inhibitors and/or interferon-based therapy, may also be used in special circumstances, for example, for non-HCV patients receiving HCV (+) donor allografts, which have been necessitated in recipients with HCV due to the worsening organ donor shortage. It has been reported that short-term patient and graft survival are similar for HCV (+) patients receiving HCV (+) donor allografts compared with a cohort of HCV (+) recipients receiving HCV (−) allografts (Schiano T D, Martin P. 2006, Int J Med Sci., 3:79-83).

In an embodiment for the management of HCV infection after transplant surgery, one or more compounds of the invention are used as anti-HCV therapy to treat a transplant patient early after liver transplant at a time when the patient may experience increased susceptibility to infection and rejection and may be prone to anemia and renal dysfunction that lessen the patient's tolerance for interferon and ribavirin. In another embodiment, the invention encompasses liver transplant treatment of patients involving an effective amount of one or more compounds of the invention administered to the patient prior to, at the time of, or following liver transplantation. In another embodiment, the invention encompasses treatment of liver transplant patients involving the use of an effective amount of one or more compounds of the invention in combination with the same medications as those used for general HCV treatment, such as interferon, e.g., peginterferon, and ribavirin. In other embodiments, the compounds of the present invention are used in combination with other small molecule anti-HCV medications or compounds to treat a patient who has undergone liver graft or transplant surgery. In other embodiment, patients are treated with a compound of the invention, with or without combination therapy, for 1-24 months, 3-15 months, 6-12 months, or 12 months following liver transplant and are subjected to monthly laboratory testing to exclude acute and chronic rejection.

In an embodiment, the invention encompasses a method of reducing or preventing HCV infection or recurrence in a liver transplant patient, which involves administering to the patient one or more compounds of the invention, or a composition containing one or more compounds of the invention, in an amount effective to reduce or prevent HCV infection or recurrence in the liver transplant patient. In various embodiments, a compound is administered to the patient at a time prior to, at the time of, or following the liver transplant. In an embodiment, the compound may be administered to a patient in a combination of modes, for example, prior to the liver transplant and at the time of transplant; or at the time of transplant and post-transplant; or prior to transplant, at the time of transplant and post-transplant. In an embodiment, a compound may be administered to the patient in combination with at least one other antiviral drug or therapeutic, for example and without limitation, other anti-HCV compounds or drugs, HCV protease inhibitors, HCV polymerase inhibitors, HCV helicase inhibitors, such as an interferon-alpha, pegylated interferon-alpha, ribavirin, or a combination thereof. In an embodiment, a compound may be administered to a patient in combination with at least one other pharmaceutical agent that is not an antiviral agent, such as an antimicrobial or anti-infective drug or therapeutic or an anti-cancer drug or therapeutic, etc.

It is desirable that an HCV inhibitory compound demonstrate one or more characteristics in order to provide improved results in HCV treatment, such as improved efficacy in the major patient population (genotype 1) as measured by an increase in the sustained virologic response (SVR) rate (as defined as HCV negative compared with the standard of care alone); improved safety and tolerability with fewer or more benign side effects compared with the standard of care; ability to shorten the duration of treatment compared with the standard of care alone; ability to replace current non-specific agents while maintaining or improving SVR rates; improved efficacy in difficult-to-treat patient groups, e.g., treatment failures (non-responders, relapsers and viral breakthroughs), African American and transplant patients; high genetic barrier to resistance; and complementary mechanism of action and resistance profile in connection with current HCV drugs.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### EXAMPLES

### Synthesis of Intermediates and Final Compounds

The following general examples illustrate the synthesis of compounds of the present invention, and should not be construed to limit the scope of the claims which follow. Variations of the synthetic procedures to produce the same or similar compounds in a somewhat different manner should be evident to one skilled in the art. Also, the procedures and methods may be suitably adapted to produce the compounds of this invention, including those not specifically disclosed.

Common abbreviations used to identify the chemical compounds and chemical techniques used herein include: diameter (d), diisopropylethylamine (DIPEA), dichloromethane (DCM), trifluoroacetic acid (TFA) thin-layer chromatography (TLC), high performance liquid chromatography (HPLC), photodiode array (PDA), evaporative light-scattering detector (ELSD), tetrahydrofuran (THF), hexa-deuterio dimethylsulfide (DMSO-d₆), proton (H), Hertz (Hz), liquid chromatography-mass spectrometry (LCMS), high performance liquid chromatography (HPLC), retention time (t_{R}), mass spectrum (MS), total ion current (TIC), atmospheric pressure chemical ionization (APCI), coupling constant, Hz (J), mass of parent ion+1 proton ([M+H]), charge to mass ratio (m/z), melting point (Mp), mega-Hertz (MHZ)., δ, ¹H NMR chemical shift, is reported in ppm.

Exemplary chemical intermediates and compounds of the present invention were prepared according to the methods that follow.

### Section 1. Generic Procedures

There are six approaches to make triazine derivatives.

### 1. R2-R4-R6

### 1.1. R2

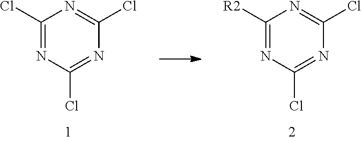

R2=alkoxy

A mixture of cyanuric chloride (1) (5.00 g, 27.1 mmol), NaHCO₃ (2.34 g, 27.8 mmol) and ethanol (30 mL) was stirred at 0° C. for 1 hour and at room temperature for 5 hours and poured onto ice. The formed solid was collected by filtration and washed with water giving compound 2.

In case of using 2,2,2-trifluoroethanol a mixture of the target product and disubstituted byproduct with ratio 1/1 was obtained. The mixture could be used on the next stage with removing of disubstituted byproduct on the last stage.

R2=alkyl

n-Propylbromide (1.23 g, 10 mmol) was added to a mixture of magnesium (2.43 g, 100 mmol) and ether (50 mL) under argon atmosphere. After start of the reaction additional portion of n-propylbromide (11.07 g, 90 mmol) was added dropwise to the reaction mixture. The obtained mixture was stirred at refluxing for 30 minutes and left overnight at room temperature under argon atmosphere. The obtained solution was added to a solution of cyanuric chloride (1) (4.15 g, 22.5 mmol) in DCM (100 mL) at −20° C. The mixture was stirred at the same temperature for 4 hours. Water (12 mL) was added dropwise keeping temperature of the reaction mixture below −10° C. The organic layer was separated and diluted with water. The formed solid was filtered off, the filtrate was extracted with dichloromethane. The combined organic layers were dried over sodium sulfate and concentrated at reduced pressure giving compound 2 stored in refrigerator and used on the next stage without additional purification.

### 1.2. R2-R4

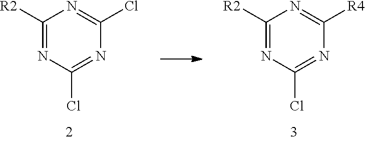

### R4=N-aryl

A solution of DIPEA (387 mg, 3 mmol) and corresponding aniline (3 mmol) in THF (5 mL) was added to a solution of compound 2 (3 mmol) in THF (5 mL) dropwise at 0° C. The obtained mixture was stirred at 0° C. or at room temperature for 1-3 hours (TLC control), diluted with water and extracted with dichloromethane. The combined organic phases were dried over sodium sulfate, concentrated and dried. The obtained residue was used on the next stage without additional purification or purified by column chromatography or recrystallization giving compound 3.

### 1.3. R2-R4-R6

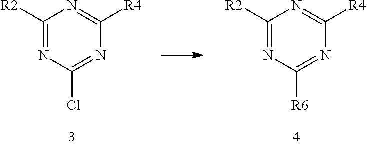

### R6=N—R

### Method A

A mixture of compound 3 (1.78 mmol), corresponding amine (1.78 mmol), DIPEA (230 mg, 1.78 mmol) and THF (10 mL) was stirred at room temperature or at 50° C. or at 100° C. for 2-4 hours (TLC control), cooled to room temperature, concentrated and subjected to preparative TLC to give compounds 4.

### Method B

A mixture of compound 3 (1.0 mmol), corresponding amine (1.1 mmol), DIPEA or NEt₃ (1.1 mmol) and acetonitrile (5 mL) was stirred at room temperature or at 50° C. or refluxing for up to 8 hours (TLC control), cooled down to room temperature, diluted with water, filtered or extracted with chloroform. The combined organic phases were concentrated at reduced pressure. Purification by appropriate method furnished compound 4.

### Method C

A mixture of compound 3 (1.5 mmol), corresponding amine (1.5 mmol), K₂CO₃ (417 mg, 3.0 mmol) and DMSO (0.2 mL) was stirred at 60-100° C. for 3-16 hours (TLC control), cooled down to room temperature, diluted with water and extracted with dichloromethane. The combined organic phases were concentrated at reduced pressure. Purification by appropriate method furnished compound 4.

### Method D

DIPEA or NEt₃ (108 mg, 1 mmol) was added to a solution of compound 3 (1 mmol) and corresponding amine (1 mmol) in dioxane (15 mL). The obtained mixture was stirred at 50° C. for 3-16 hours (TLC control), diluted with water and extracted with ethyl acetate. The combined organic phases were dried over sodium sulfate and concentrated. Purification by column chromatography on silica gel or by recrystallization from appropriate solvents gave compound. 4

### R6=O-alkyl

Sodium hydride (60% in oil, 60 mg, 1.5 mmol) was dissolved in a solution of corresponding alcohol (1.5 mmol) in THF (5 mL) at 0° C. To the obtained solution a solution of compound 3 (1.5 mmol) in THF (15 mL) was added at 0° C. The resulting mixture was stirred at 0° C. for 30 minutes, then at room temperature for 1 hour and at 50° C. for 4 hours, cooled to room temperature, diluted with water and extracted with dichloromethane. The combined organic phases were dried over sodium sulfate and concentrated at reduced pressure. Purification by column chromatography furnished compound 4.

### 2. R2-R6-R4

### 2.1. R2 See Item 1.1.

### 2.2. R2-R6

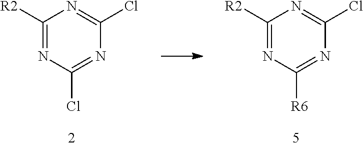

For such transformation (R2-R6) the same procedure as in item 4.2 was applied.

### 2.3. R2-R6-R4

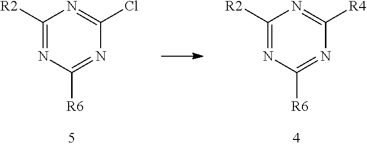

For such transformation (R2-R6-R4) the same procedure as in item 5.3 was applied.

### 3. R4-R2-R6

### 3.1. R4

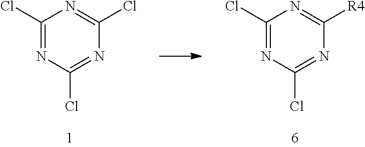

### R4=N-aryl

To a solution of cyanuric chloride (1) (5.00 g, 27 mmol) in THF (50 mL) a solution of corresponding aniline (27 mmol) and DIPEA (3.50 g, 27 mmol) in THF (50 mL) was added slowly dropwise at −20° C. The resulting mixture was stirred at −20° C. for 2 hours (TLC control), warmed up to room temperature and concentrated at reduced pressure. Purification by column chromatography on silica gel gave compound 6.

### 3.2. R4-R2

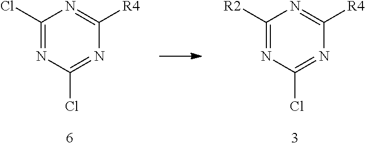

R2=alkoxy

### Method A

Sodium (1.20 g, 52 mmol) was dissolved in anhydrous alcohol (20 mL) at room temperature. The obtained solution was added slowly dropwise to a precooled to 0° C. solution of compound 6 (52 mmol) in THF (50 mL). The resulting mixture was stirred at 0° C. for 1 hour (TLC control), warmed up to room temperature, concentrated at reduced pressure, diluted with dichloromethane and washed with water. The organic phase was concentrated at reduced pressure. The obtained product was recrystallized to give compound 3.

### Method B

Sodium (250 mg, 10.8 mmol) was dissolved in a mixture of 2,2,2-trifluoroethanol (1.63 g, 16.3 mmol) and THF (10 mL). The obtained solution was added to a solution of crude compound 6 (10.8 mmol) in THF (10 mL) dropwise at 0° C. The mixture was stirred at 0° C. for 2 hours and warmed up to room temperature. The solvent was removed at reduced pressure. The residue was treated with water and extracted with chloroform. The combined organic phases were dried over sodium sulfate and concentrated at reduced pressure. Recrystallization from an appropriate solvent gave compound 3.

### R2=N-alkyl (HNEt)

A mixture of compound 6 (1.84 mmol), ethylamine hydrochloride (144 mg, 1.84 mmol), DIPEA (0.641 mL, 3.68 mmol) and THF (15 mL) was stirred at 50° C. for 11 hours, cooled to room temperature and diluted with water. The formed solid was collected by filtration and washed with methanol to give compound 3.

### 3.3. R4-R2-R6 See Item 1.3

### 4. R4-R6-R2

### 4.1. R4 See Item 3.1.

### 4.2. R4-R6

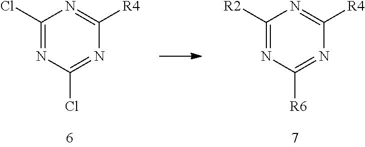

### R6=N-alkyl

To a solution of compound 6 (1.85 mmol) in THF (20 mL) a solution of corresponding amine (1.85-2.03 mmol) and triethylamine or DIPEA (3.7 mmol) in THF (5 mL) was added. The mixture was stirred at 0° C. or at room temperature for 30 minutes-3 hours (TLC control), diluted with water and extracted with ethyl acetate. The combined organic phases were dried over sodium sulfate and concentrated. The residue was purified by column chromatography and crystallized to give compound 7.

Note: if starting compound 6 has low solubility in THF, DMSO could be used as a solvent or ethanol could be added to THF as a co-solvent.

### 4.3. R4-R6-R2

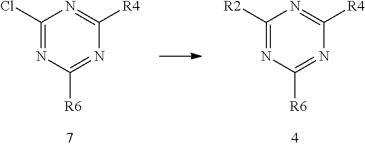

### R2=N—R

### Method A

A mixture of compound 7 (1 mmol), corresponding aniline (1 mmol), anhydrous K₂CO₃ (415 mg, 3 mmol) and DMSO (200 μL) was heated at 150° C. for 2 hours. After cooling to room temperature the resulting mixture was washed with water and extracted with ethyl acetate. The combined organic phases were dried over Na₂SO₄ and concentrated at reduced pressure. Purification by column chromatography on silica gel or by other appropriate methods of purification gave compound 4.

### Method B

PBu^{t}₃ (0.2 mg, 7 mol %) was added to a solution of Pd₂(dba)₃ (37 mg, 5 mol %) in toluene (1 mL). The solution was stirred at room temperature for 15 minutes. Then it was transferred to a solution of compound 7 (0.78 mmol), corresponding aniline (1.57 mmol) and NaOBu^{t} (160 mg, 1.7 mmol) in toluene (3 mL). The obtained mixture was stirred at refluxing for 4 hours, diluted with water and extracted with ethyl acetate. The combined organic fractions were dried over sodium sulfate and concentrated. Purification by column chromatography on silica gel gave compound 4.

### Method C

A mixture of compound 7 (0.78 mmol), corresponding amine (3.7 mmol) and DIPEA (5 mL) was stirred for 2 hours at 100° C., cooled down to room temperature and diluted with water. The formed solid was collected by filtration and recrystallized from appropriate solvent to give compound 4.

### Method D

A mixture of compound 7 (0.3 mmol) and corresponding amine (2.3 mmol) was stirred at 120° C. for 1 hour (TLC control), cooled to room temperature and diluted with water. The formed solid was collected by filtration, washed with water and diethyl ether to give compound 4.

### Method E

A mixture of compound 7 (0.4 mmol), corresponding amine (2 mmol), triethylamine (0.14 mL, 1 mmol) and ethanol (2 mL) was stirred at refluxing for 1 hour (TLC control), cooled down to room temperature and diluted with water. The formed solid was collected by filtration, washed with water and diethyl ether to give compound 4.

### Method F

A mixture of compound 7 (0.81 mmol), corresponding amine (0.81 mmol), triethylamine or DIPEA (0.89 mmol) and acetonitrile (4 mL) was stirred at 50-70° C. for 4-12 hours (TLC control), cooled down to room temperature and concentrated at reduced pressure. The residue was purified by column chromatography to give compound 4.

### Method G

A mixture of compound 7 (1.0 mmol), corresponding amine (1.0 mmol), K₂CO₃ (417 mg, 3.0 mmol) and dioxane (5 mL) was stirred at 80° C. for 8 hours and cooled to room temperature. Then the obtained mixture was transferred to column directly. Purification by column chromatography and by preparative TLC gave compound 4.

### R2=O-alkyl

### Method A

Sodium (46 mg, 2.0 mmol) was dissolved in alcohol (1 mL) at room temperature. The obtained solution was added dropwise to a solution of compound 7 (1.0 mmol) in anhydrous ethanol (3 mL). The resulting mixture was stirred at room temperature for 20 minutes, and then at refluxing for 2 hours. After completion of the reaction (TLC control) the solvent was removed at reduced pressure. The residue was washed with water and extracted with chloroform. The combined organic phases were concentrated at reduced pressure. Purification by column chromatography on silica gel gave compounds 4.

### Method B

Sodium hydride (59 mg, 60% in oil, 1.5 mmol) was added to a solution of corresponding alcohol (1.5 mmol) in THF (5 mL) at 0° C. The mixture was stirred for 15 minutes at 0° C. Then a solution of compound 7 (1.5 mmol) in THF (15 mL) was added to the obtained suspension at 0° C. The final reaction mixture was stirred at room temperature for 1 hour and at 50° C. for 3.5 hours, cooled to room temperature, diluted with water and extracted with dichloromethane. The combined organic phases were concentrated at reduced pressure and purified by column chromatography to give compound 4.

### Method C

Sodium hydride (68 mg, 60% in oil, 1.7 mmol) was added to a solution of alcohol (1.7 mmol) in THF (2 mL) at 0° C. The mixture was stirred for 20 minutes at 0° C. Then a solution of compound 7 (0.81 mmol) in THF (2 mL) was added to the obtained suspension at 0° C. The final reaction mixture was stirred at room temperature for 8 hours or at refluxing for 15 minutes, diluted with water and extracted with dichloromethane. The combined organic phases were concentrated at reduced pressure and purified by column chromatography to give compounds 4.

### Method D

Sodium hydride (60% in oil, 117 mg, 3.0 mmol) was added slowly to a solution of alcohol (2.6 mmol) in DMF (5 mL) at 0° C. The obtained mixture was allowed to warm up to room temperature. Compound 7 (1.3 mmol) was added to the mixture at room temperature and the resulting mixture was stirred at room temperature for 4 hours and diluted with water. The formed solid was collected by filtration and purified by appropriate method giving compound 4.

### Method E

A mixture of compound 7 (1.0 mmol), 2,2,2-trifluoroethanol (1 mL), DMSO (3 L) and K₂CO₃ (500 mg) was stirred at 100° C. for 2.5 hours, cooled to room temperature and diluted with water. The formed solid was collected by filtration and recrystallized from appropriate solvent giving compound 4.

### Method F

Sodium hydride (60% in oil, 150 mg, 3.8 mmol) was dissolved in 2,2,2-trifluoroethanol (3 mL). Then compound 7 (0.5 mmol) was added to the obtained solution. The resulting mixture was stirred at refluxing for 4 hours, cooled to room temperature and diluted with water. The formed solid was collected by filtration, washed with water and diethyl ether and purified by preparative TLC giving compound 4.

### R2=O-aryl

### Method A

Sodium hydride (60% in oil, 24 mg, 0.6 mmol) was added portionwise to a solution of a phenol (0.6 mmol) in DMF (5 mL) at 0° C. The obtained mixture was stirred at 0° C. for 5 minutes. Then compound 7 (0.5 mmol) was added portionwise at 0° C. The resulting mixture was stirred at 0° C. for 30 minutes and for 2 hours at room temperature and diluted with water. The formed solid was collected by filtration and purified by column chromatography to give compound 4.

### Method B

A mixture of compound 7 (0.78 mmol), p-hydroxyphenol (257 mg, 2.34 mmol), potassium carbonate (414 mg, 3 mmol) and DMSO (200 μl) was stirred for 4 hours at 180° C., diluted with water and extracted with ethyl acetate. The combined organic phases were concentrated at reduced pressure. Purification by column chromatography gave compound 4.

R2=aryl(Het)

A mixture of compound 7 (1.0 mmol), boronic acid (1.0 mmol), Pd(PPh₃)₄ (57 g, 0.05 mol), Na₂CO₃ (430 mg, 4.0 mmol), dimethoxy ethane (2 mL) and water (2 mL) was stirred at refluxing for 4-6 hours (TLC control), cooled to room temperature, diluted with water and extracted with ethyl acetate. The combined organic phases were dried over sodium sulfate and concentrated. Purification by column chromatography and preparative TLC gave a final compound 4.

### R2=CN

A mixture of compound 7 (12.17 mmol), NaCN (2.98 g, 60.85 mmol) and DMSO (40 mL) was stirred at 60-80° C. for 3-12 hours (LCMS control of the reaction), cooled to room temperature and diluted with water. The formed solid was collected by filtration and purified by column chromatography to give compound 4.

### R2=0H

A mixture of compound 7 (1 mmol), sodium hydroxide (0.12 g, 3 mmol) and water (10 mL) was stirred at refluxing for 4 hours, cooled to room temperature and neutralized with concentrated HCl aqueous solution to reach pH 2. The formed solid was collected by filtration, washed with water and cold ethanol to give compound 4.

### 5. R6-R2-R4

### 5.1. R6

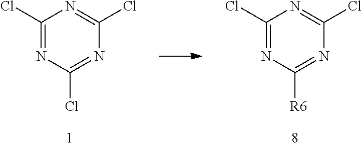

### R6=N-alkyl

To a cooled to −20° C. solution of cyanuric chloride (1) (5.50 g, 30 mmol) and N,N-diisopropylethylamine (DIPEA) or triethylamine (30 mmol) in THF (60 mL) a solution of corresponding amine (30 mmol) in THF (60 mL) was added slowly dropwise at −20° C. The resulting mixture was stirred at −20° C. for 30 minutes (TLC control) and allowed to warm up to room temperature. Then the reaction mixture was transferred on the column directly or quenched with water and extracted with dichloromethane or ethyl acetate. Purification by column chromatography or by recrystallization gave compound 8

### 5.2. R6-R2 See Item 3.2

### 5.3. R6-R2-R4

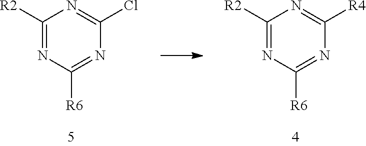

### R4=N—R

### Method A

A mixture of compound 5 (0.4 mmol), corresponding aniline (0.4 mmol), K₂CO₃ (170 mg, 1.2 mmol) and DMF (2.5 mL) was stirred at 100-150° C. for 2-4 hours (TLC control), cooled down to room temperature and diluted with water. The formed solid was collected by filtration. Purification by column chromatography on silica gel or by other appropriate methods furnished compound 4.

### Method B

A mixture of compound 5 (1.0 mmol), corresponding aniline (1.0 mmol), K₂CO₃ (400 mg, 3 mmol) and DMSO (5.0 mL) was stirred at 80-150° C. for 2-4 hours (TLC control), cooled down to room temperature and diluted with water. The formed solid was collected by filtration or the reaction mixture was extracted with dichloromethane. Purification by column chromatography on silica gel or by other appropriate methods furnished a final compound 4.

### Method C

A mixture of compound 5 (2 mmol), corresponding amine (2 mmol), KOH (168 mg, 3 mmol) and acetone (5 mL) was stirred at refluxing for 20 hours, diluted with water and extracted with ethyl acetate. The combined organic phases were concentrated. Purification by column chromatography gave compound 4.

### Method D

A mixture of compound 5 (1.0 mmol), corresponding amine (1.0 mmol), DIPEA (194 mg, 1.5 mmol) and dioxane (5 mL) was stirred at 70° C. for 8 hours and cooled down to room temperature. Then the obtained mixture was transferred to column and purified by column chromatography. Additional purification by preparative TLC gave a final compound 4.

### Method E

A mixture of compound 5 (1.18 mmol), corresponding aniline (1.18 mmol), DIPEA (460 mg, 3.54 mmol) and acetonitrile (6 mL) was stirred at room temperature for 4 hours and diluted with water. The formed solid was collected by filtration. Purification by prepTLC provided compound 4.

### Method F

A mixture of compound 5 (1.18 mmol), corresponding amine (1.24 mmol), NEt₃ (0.174 mL, 1.24 mmol) and acetonitrile (10 mL) was stirred at room temperature for 2 hours. Then DMSO (2 mL) was added to the reaction mixture. The obtained mixture was stirred at 100° C. for 14 hours, cooled to room temperature. The formed solid was collected by filtration and recrystallized giving compound 4.

### Method G

To a solution of compound 5 (1 mmol) in acetic acid (3 mL) sodium acetate (100 mg, 1.22 mmol) and corresponding amine (1.15 mmol) were added. The mixture was stirred at 50° C.-90° C. for 3 hours, cooled down to room temperature, neutralized with aqueous ammonia solution and extracted with ethyl acetate. The organic phases were combined, dried over sodium sulfate and concentrated. The residue was purified by column chromatography and recrystallized giving compound 4.

### Method H

A mixture of compound 5 (1.18 mmol), corresponding amine (1.30 mmol), NEt₃ or DIPEA (3.54 mmol) and acetonitrile (6 mL) was stirred at refluxing for 3 hours, cooled to room temperature, diluted with water and extracted with chloroform. The combined organic phases were concentrated. Purification by column chromatography gave compound 4.

### Method I

Corresponding amine (1.56 mmol) and NaHCO₃ (150 mg, 1.79 mmol) were added to a solution of compound 5 (1.18 mmol) in ethanol (5 mL). The mixture was stirred at refluxing for 1 hour, cooled to room temperature, diluted with water and extracted with chloroform. The combined organic phases were dried over sodium sulfate and concentrated at reduced pressure. Purification by column chromatography gave compound 4.

### Method J

A mixture of compound 5 (0.94 mmol), corresponding amine (1.13 mmol), NaHCO₃ (95 mg, 1.13 mmol) or K₂CO₃ (156 mg, 1.13 mmol) and acetonitrile (3 mL) was stirred at 60° C. or at refluxing for 2-24 hours (TLC control). The mixture was diluted with water, extracted with chloroform, dried over sodium sulfate and concentrated. The residue was purified by column chromatography or preparative TLC giving compound 4.

### Method K

A mixture of compound 5 (2 mmol), corresponding amine (2 mmol), DIPEA (387 mg, 3 mmol) and THF (5 mL) was stirred at 50° C. for 5 hours, cooled to room temperature, washed with water, dried over sodium sulfate and concentrated at reduced pressure. Purification by appropriate method gave compound 4.

### Method L

A mixture of PBu^{t}₃ (500 mg, 2.47 mmol), Pd(OAc)₂ (40 mg, 0.18 mmol), compound 5 (1.57 mmol), corresponding amine (1.88 mmol), K₂CO₃ (325 mg, 2.36 mmol) and toluene (5 mL) was stirred at refluxing for 3 hours under argon atmosphere, cooled to room temperature and concentrated. Purification by column chromatography on silica gel gave compound 4.

R4=aryl

A mixture of compound 5 (1.0 mmol), boronic acid (1.0 mmol), Pd(PPh₃)₄ (120 mg, 0.1 mol, 10 mol %), Na₂CO₃ (424 mg, 4.0 mmol), dimethoxy ethane (3 mL) and water (3 mL) was stirred at refluxing for 3-24 hours (TLC control), cooled to room temperature, filtered through a pad of Celite and extracted with ethyl acetate. The combined organic phases were dried over sodium sulfate and concentrated. Purification by column chromatography gave compound 4.

### R4=CN

A mixture of compound 5 (4.71 mmol), NaCN (1.155 g, 23.56 mmol) and DMSO (12 mL) was stirred at 60° C. for 4 hours, cooled to room temperature, diluted with water and extracted with dichloromethane. The combined organic phases were washed with brine, dried over sodium sulfate and purified by column chromatography giving compound 4.

### R4=O-aryl

Sodium hydride (60% in oil, 24 mg, 0.6 mmol) was added portionwise to a solution of phenol (0.6 mmol) in DMF (5 mL) at 0° C. The obtained mixture was stirred at 0° C. for 5 minutes. Then compound 5 (0.5 mmol) was added portionwise at 0° C. The resulting mixture was stirred at 0° C. for 30 minutes and for 2 hours at room temperature and diluted with water. The formed solid was collected by filtration and purified by column chromatography to give compound 4.

### R4=O-alkyl

A solution of corresponding alcohol (2.4 mmol) in THF (1 mL) was added to a suspension of sodium hydride (60% in oil, 96 mg, 2.4 mmol) in THF (3 mL). The mixture was stirred at room temperature for 30 minutes. A solution of compound 5 (1.18 mmol) in THF (2 mL) was added to the obtained mixture and the resulting mixture was stirred at refluxing for 2 hours, cooled to room temperature, poured into water and extracted with chloroform. The combined organic phases were washed with water, dried over sodium sulfate and concentrated. The residue was purified by column chromatography giving compound 4.

### 6. R6-R4-R2

### 6.1. R6 See Item 5.1.

### 6.2. R6-R4 See Item 1.2

### 6.3. R6-R4-R2 See Item 4.3

### General Analytical Characterization and Purification Procedures:

Column chromatography: silica gel Sorbfil 40-60 A; l=10 cm; d=2.5 cm.

Preparative TLC was done on Silica Gel 60 F254 plates 200×200×2 mm (EMD).

HPLC analyses for the final compounds were done on the Finnigan instrument. Column: Nova-Pak C18 3.9×150 mm 5 mkm column, Mobile Phase: acetonitrile/(water+tfa 0.05%) 5/95 (0 min)-100/0 (25 min)-100/0 (35 min). Flow: 1.0 ml/min. Column Temperature: ambient. Detection: UV at 220 nm and 254 nm.

NMR spectra were registered on <<MERCURY plus 400 MHz>> spectrometer (Varian). Chemical shift values are given in ppm relative to tetramethylsilane (TMS), with the residual solvent proton resonance as internal standard. All samples were dissolved in DMSO-D₆ or in CDCl₃. Peak broadening and splitting results from the restricted rotation around the specified bond.

LC/MS analyses for the compounds were done on the following instruments

Surveyor MSQ (Thermo Finnigan, USA) with APCI ionization. Type of HPLC column: Waters XTerra MS C18 3.5 um 2.1×30 mm. Solvent: 50% DMSO, 50% acetonitrile. Flow rate—1.5 ml/min. Column temperature 25° C. Mobil phase: A—0.1% solution of formic acid; B—acetonitrile.

Total run time −4.5 min. Gradient:

| time, min. | A % | B % |
|---|---|---|
| 0.0 | 100 | 0 |
| 0.1 | 100 | 0 |
| 2.1 | 5 | 95 |
| 2.5 | 5 | 95 |
| 2.6 | 100 | 0 |
| 4.0 | 100 | 0 |

Total run time −6.0 min. Gradient:

| time, min. | A % | B % |
|---|---|---|
| 0.0 | 100 | 0 |
| 0.1 | 100 | 0 |
| 3.3 | 5 | 95 |
| 3.9 | 5 | 95 |
| 4.0 | 100 | 0 |
| 5.5 | 100 | 0 |

Detection: diode array (PDA), 190-800 nm; photodiode array detector. Detection was carried out in the full ultraviolet-visible range from 190 to 800 nm. APCI (+ or − ions)—atmospheric pressure chemical ionization. TIC—total ion current. ELSD (PL-ELS 2100)—evaporated light scattering detector. Injection volume: 1 μl.

### Standard LC-MS Analysis

### Sample Preparation:

Compound is dissolved in a solvent mixture of DMSO, acetonitrile and water at 0.5 mg/ml concentration.

Prepared solution is transferred into standard 1.4 ml minitube.

The minitube with ˜100 μl of the analyzed solution is placed into Matrix minitube rack and submitted for analysis.

### Instrumentation:

Shimadzu Analytical HPLC with Gilson 215 autosampler and Dual UV wavelength detection, in tandem with Sedex 75 ELSD and PESCIEX API 150EX mass spectrometer.

### Standard LCMS System Chromatographic and Mass Spectrometer Parameters:

Column: Synergi 2μ Hydro-RPMercury, 20×2.0 mm;

Sample injection, μL—3.0-5.0 (depending on system settings);

Solvent A—water with 0.05% of TFA;

Solvent B—acetonitrile with 0.05% TEA;

Gradient time programs: A: B

| | |
|---|---|
| Program 1 (routine analysis) | Program 2 (research samples) |

| Time, min | % B | Time, min | % B |
|---|---|---|---|
| 0.01 | 8 | 0.01 | 8 |
| 2.90 | 95 | 2.20 | 95 |
| 3.70 | 95 | 2.75 | 95 |
| 3.95 | 5 | 2.95 | 5 |
| 4.02 | Stop | 3.02 | Stop |

Pause time for re-equilibration of column 40 s;

Flow rate 0.50 mL/min;

UV detection wavelength, nM—215, 254;

Mass range, m/z—100 . . . 1000 positive mode.

### Standard ¹H NMR Analysis of Library Samples

### Sample Preparation:

3 mg of Compound is dissolved in 500 microliters of appropriate deuterated solvent (DMSO-d6 or CDCl₃) and transferred into a 5 mm NMR tube.

Tube with prepared solution of analyzed compound is submitted for analysis.

### Instrumentation:

The ¹H NMR is conducted on a 400 MHz Bruker DPX Instrument using the following parameters:

sw=8000 Hz; np=32768; fn=64000; ns=1; temp=25° C.

### Data Processing:

The spectrum is processed using Bruker XWinNMR software.

Data is fourier transformed without weighting functions, phased, and integrated.

Printouts of 0-10 ppm are standard; if peaks are visible in the 10-18 ppm range, then the full spectrum is printed.

The raw data is stored electronically using XWinNMR software.

Spectrum analysis and final EMF files are created using ADVASP software.

The final data is submitted according to the project specification in one of the following formats: EMF, FID or printed hardcopy of the spectrum.

### Exemplary Synthesis of Intermediates

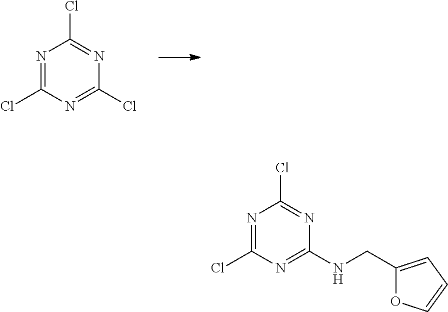

### 2-(Furan-2-ylmethyl-amino)-4,6-dichloro-[1,3,5]triazine (I-1)

To a cooled to −20° C. solution of cyanuric chloride (5.50 g, 30 mmol) and N,N-diisopropylethylamine (3.90 g, 30 mmol) in THF (60 mL) a solution of furfurylamine (2.9 g, 30 mmol) in THF (60 mL) was added slowly dropwise at −20° C. The resulting mixture was stirred at −20° C. for 30 minutes (TLC control), allowed to warm up to room temperature and transferred on the column. Purification by column chromatography on silica gel (ethyl acetate/hexane) gave 1 (6.62 g, 90%).

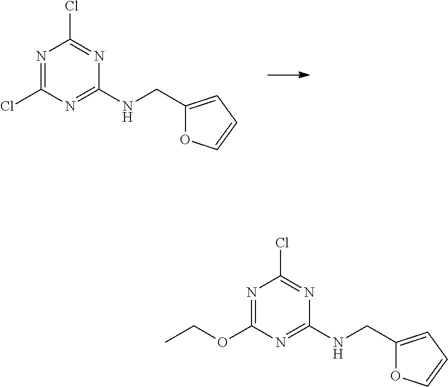

### 2-(Furan-2-ylmethyl-amino)-4-Chloro-6-ethoxy-[1,3,5]triazine (I-2)

Sodium (1.20 g, 52 mmol) was dissolved in anhydrous ethanol (20 mL) at room temperature. The obtained solution was added slowly dropwise to a precooled to 0° C. solution of compound 3 (12.77 g, 52 mmol) in THF (50 mL). The resulting mixture was stirred at 0° C. for 1 hour (TLC control), warmed up to room temperature, concentrated at reduced pressure, diluted with dichloromethane, washed with water. The organic phase was concentrated at reduced pressure. The obtained product was recrystallized from dichloromethane/hexane to give compound 4.

Yield 11.90 g, 90%. ¹H-NMR (400 MHz, DMSO-D₆) δ_{H}: 1.30 (3H, t, J=7.5 Hz), 4.28-4.42 (2H, two q, J=7.5 Hz, Z/E forms), 4.49 (2H, broad), 6.29 (1H, broad), 6.39 (1H, broad), 7.06 (1H, s), 8.74-8.90 (1H, broad, Z/E forms). LCMS t_{R} (min): 1.72. MS (APCI), m/z 254.95, 256.95 [M+H]⁺. HPLC t_{R} (min): 12.70.

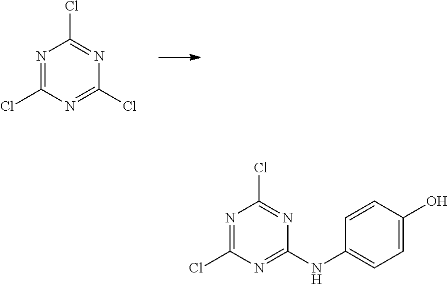

### 2-(4-Hydroxy-phenylamino)-4,6-dichloro-[1,3,5]triazine (I-3)

To a solution of cyanuric chloride (5.00 g, 27 mmol) in THF (50 mL) a solution of 4-hydroxyaniline (3.00 g, 27 mmol) and N,N-diisopropylethylamine (3.50 g, 27 mmol) in THF (50 mL) was added slowly dropwise at −30° C. The resulting mixture was stirred at −30° C. for 2 hours (TLC control), warmed up to room temperature and concentrated at reduced pressure. Purification by column chromatography on silica gel (ethyl acetate/hexane) gave 3 (5.55 g, 80%).

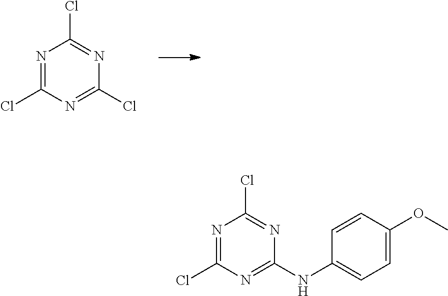

### 2-(4-Methoxy-phenylamino)-4,6-dichloro-[1,3,5]triazine (I-4)

Compound I-4 was prepared according to the procedure for I-3 (5.048 g, 69%).

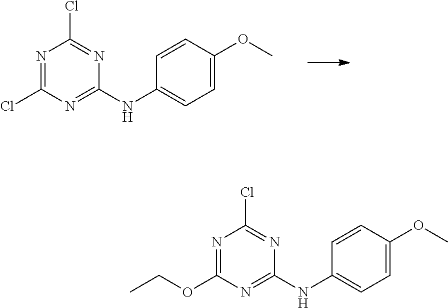

### 2-(4-methoxy-phenylamino)-4-chloro-6-ethoxy-[1,3,5]triazine (I-5)

Method A Compound I-5 was prepared according to the procedure for I-2 (2.135 g, 74%).

Method B A mixture of compound I-4 (1.50 g, 5.5 mmol), NaHCO₃ (560 mg, 6.6 mmol) and ethanol (20 mL) was stirred at room temperature for 3 hours, then at 70° C. for 3 hours (TLC control), cooled to room temperature, concentrated at reduced pressure and washed with water to give I-5 (150 mg, 10%).

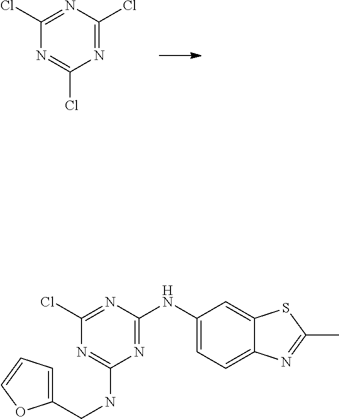

### 6-Chloro-N-furan-2-ylmethyl-N′-(2-methyl-benzothiazol-6-yl)-[1,3,5]triazine-2,4-diamine (I-6)

Compound I-6 was prepared according to the procedure for I-45 (312 mg, 29%).

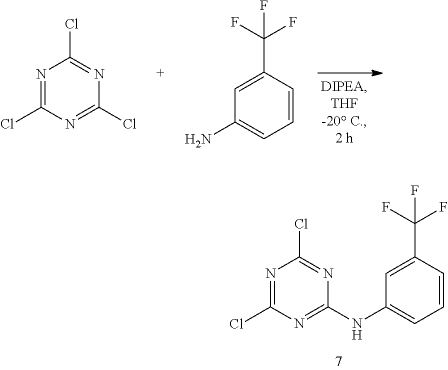

### (4,6-Dichloro-[1,3,5]triazin-2-yl)-(3-trifluoromethyl-phenyl)-amine (I-7)

To a solution of cyanuric chloride (5.00 g, 27 mmol) in THF (50 mL) a solution of 3-CF₃-aniline (4.35 g, 27 mmol) and DIPEA (3.50 g, 27 mmol) in THF (50 mL) was added slowly dropwise at −20° C. The resulting mixture was stirred at −30° C. for 2 hours (TLC control), warmed up to room temperature and concentrated at reduced pressure. Purification by column chromatography on silica gel (ethyl acetate/hexane) gave I-7 (6.87 g, 60%).

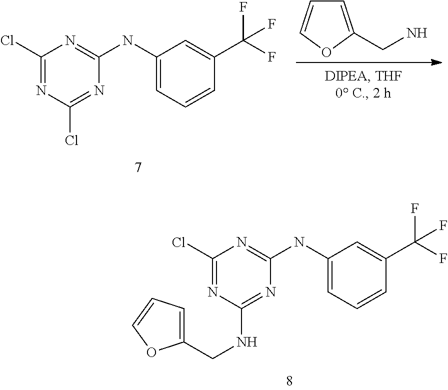

### 6-Chloro-N-furan-2-ylmethyl-N′-(3-trifluoromethyl-phenyl)-[1,3,5]triazine-2,4-diamine (I-8)

A solution of m-CF₃-aniline (1.0 g, 8.84 mmol), DIPEA (1.14 g, 8.84 mmol) in THF (10 mL) was added slowly dropwise to a solution of cyanuric chloride (1.62 g, 8.84 mmol) in THF (10 mL) at −20° C. during 30 min. Then the mixture was stirred at −20° C. for 1.5 hour and warmed up to 0° C. A solution of furfuryl amine (1.42 g, 8.84 mmol) and DIPEA (1.40 g, 8.84 mmol) in THF (10 mL) was added slowly dropwise to the resulting mixture during 30 minutes at 0° C. The obtained solution was stirred at 0° C. for 2 hours, concentrated at reduced pressure, washed with water and extracted with chloroform. The combined organic phases were concentrated at reduced pressure to give desired compound I-8 (2.94 g, 90%) used further without any additional purification.

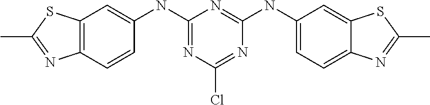

### 6-Chloro-N,N′-bis-(2-methyl-benzothiazol-6-yl)-[1,3,5]triazine-2,4-diamine (I-9)

To a solution of 1 (1.000 g, 5.4 mmol) in THF (10 mL) a solution of aniline (1.773 g, 10.8 mmol), DIPEA (1.400 g, 10.8 mmol) in THF (5 mL) was added dropwise at −30° C. The resulting mixture was stirred for 1.5 hours at −20° C., let to warm up to room temperature, stirred for 3 hours at room temperature, concentrated at reduced pressure, washed with water, extracted with chloroform and used on the next stage without additional purification.

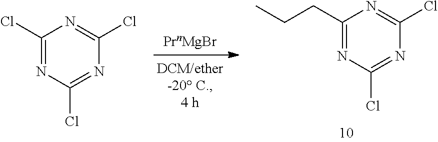

### 2,4-Dichloro-6-propyl-[1,3,5]triazine (I-10)

n-Propylbromide (1.23 g, 10 mmol) was added to a mixture of magnesium (2.43 g, 100 mmol) and ether (50 mL) under argon atmosphere, and additional portion of n-propylbromide (11.07 g, 90 mmol) was added dropwise to the reaction mixture. The obtained mixture was stirred at refluxing for 30 min and left overnight at room temperature under argon atmosphere. The obtained solution was added to a solution of cyanuric chloride (4.15 g, 22.5 mmol) in DCM (100 mL) at −20° C. The mixture was stirred at the same temperature for 4 hours. Water (12 mL) was added dropwise keeping temperature of the reaction mixture below −10° C. The organic layer was separated, diluted with water. The formed solid was filtered off, the filtrate was extracted with dichloromethane. The combined organic layers were dried over sodium sulfate and concentrated at reduced pressure giving compound I-10 stored in refrigerator and used on the next stage without additional purification. Yield 4.22 g, 98%.

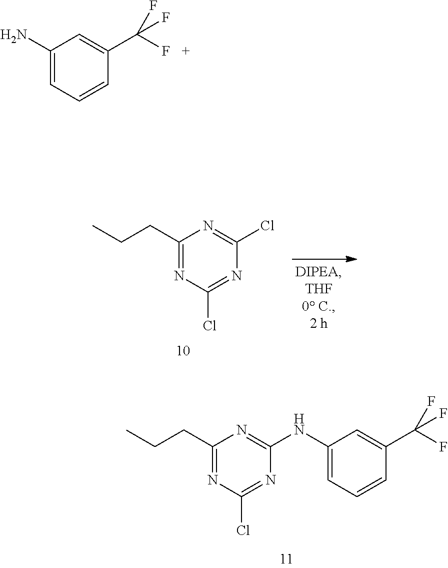

### (4-Chloro-6-propyl-[1,3,5]triazin-2-yl)-(3-trifluoromethyl-phenyl)-amine (I-11)

A mixture of m-CF₃-aniline (0.163 mL, 1.3 mmol) and DIPEA (0.226 mL, 1.3 mL) was added to a solution of compound I-10 (250 mg, 1.3 mmol) in THF (5 mL) at 0° C. The resulted mixture was stirred at 0° C. for 2 hours, diluted with water and extracted with ethyl acetate. The combined organic phases were washed with brine, dried over sodium sulfate and concentrated giving compound crude product I-11 used further without additional purification. Yield 465 mg, 98%.

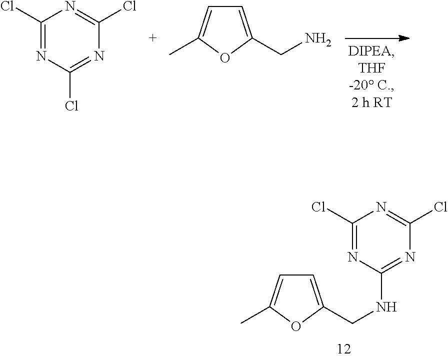

### (4,6-Dichloro-[1,3,5]triazin-2-yl)-(5-methyl-furan-2-ylmethyl)-amine (I-12)

To a cooled to −20° C. solution of cyanuric chloride (5.50 g, 30 mmol) and N,N-diisopropylethylamine (3.90 g, 30 mmol) in THF (60 mL) a solution of 5-methylfurfurylamine (3.3 g, 30 mmol) in THF (60 mL) was added slowly dropwise at −20° C. The resulting mixture was stirred at −20° C. for 30 minutes (TLC control), allowed to warm up to room temperature and transferred on the column. Purification by column chromatography on silica gel (ethyl acetate/hexane) gave compound I-12. Yield 6.602 mg, 85%.

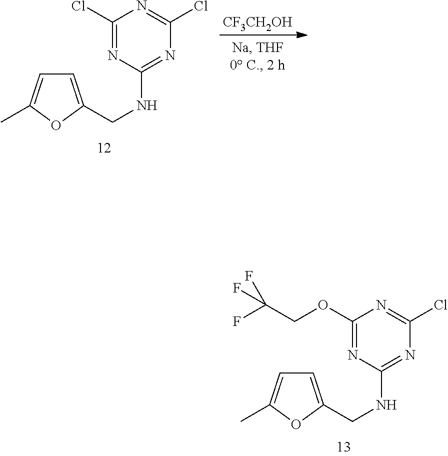

### [4-Chloro-6-(2,2,2-trifluoro-ethoxy)-[1,3,5]triazin-2-yl]-(5-methyl-furan-2-ylmethyl)-amine (I-13)

Sodium (250 mg, 10.8 mmol) was dissolved in a solution of 2,2,2-trifluoroethanol (1.63 g, 16.3 mmol) in THF (10 mL) at room temperature. Then the obtained solution was added to a solution of compound I-12 (1.40 g, 5.4 mmol) in THF (10 mL) at 0° C. The resulting mixture was stirred at 0° C. for 2 hours, warmed to room temperature and concentrated. Water was added to the residue. The mixture was extracted with chloroform. The combined organic phases were dried over sodium sulfate and concentrated and dried. Recrystallization from diethyl ether gave compound I-13 (1.47 g, 84% for two steps). ¹H-NMR (400 MHz, DMSO-D₆) δ_{H}: 2.21 (3H, s), 4.45 (2H, superposition of two d, J=7.5 Hz, Z/E forms), 4.91-5.07 (2H, two q, J=7.5 Hz, Z/E forms), 5.97 (1H, broad), 6.12-6.28 (1H, broad, Z/E forms), 8.96-9.14 (1H, broad, Z/E forms). MW 322.68. LCMS t_{R} (min): 1.96. MS (APCI), m/z 322.88, 324.86 [M+H]⁺. HPLC t_{R} (min): 14.80. M_{P} 70-72° C.

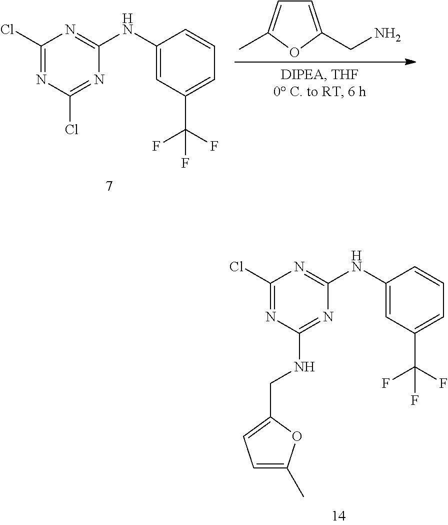

### 6-Chloro-N-(5-methyl-furan-2-ylmethyl)-1V-(3-trifluoromethyl-phenyl)-[1,3,5]triazine-2,4-diamine (I-14)

To a solution of compound 7 (309 mg, 1 mmol) in THF (3 mL) a solution of 5-methylfurfuryl amine (0.110 mL, 1 mmol), DIPEA (0.130 mL, 1 mmol) in THF (3 mL) was added at 0° C. The obtained mixture was stirred at 0° C. for 1 hour, then warmed up to room temperature and stirred at room temperature for 5 hours. The solvent was removed at reduced pressure. The residue was diluted with water and extracted with dichloromethane. The combined organic phases were concentrated and dried giving final compound I-14 (335 mg, 87%).

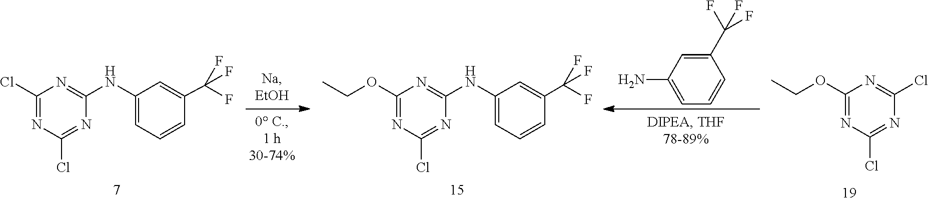

### 2-(4-methoxy-phenylamino)-4-chloro-6-ethoxy-[1,3,5]triazine (I-15)

Sodium (1.20 g, 52 mmol) was dissolved in anhydrous ethanol (20 mL) at room temperature. The obtained solution was added slowly dropwise to a precooled to 0° C. solution of compound I-14 (1.61 g, 52 mmol) in THF (50 mL). The resulting mixture was stirred at 0° C. for 1 hour (TLC control), warmed up to room temperature, concentrated at reduced pressure, diluted with dichloromethane, washed with water. The organic phase was concentrated at reduced pressure. The obtained product was recrystallized from dichloromethane/hexane to give 15 (4.00 g, 30%)

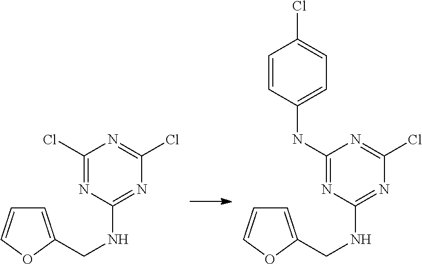

### 6-Chloro-N-(4-chloro-phenyl)-N′-furan-2-ylmethyl-[1,3,5]triazine-2,4-diamine (I-16)

Compound I-16 (1.06 g, 67%) was prepared according to procedure for I-45.

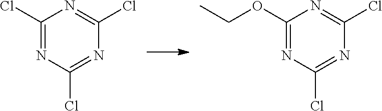

### 2,4-Dichloro-6-ethoxy-[1,3,5]triazine (I-19)

A mixture of cyanuric chloride (5.00 g, 27.1 mmol), K₂CO₃ (2.34 g, 27.8 mmol) and ethanol (30 mL) was stirred at 0° C. for 1 hour and at room temperature for 5 hours, poured onto ice. The formed solid was collected by filtration and washed with water to give compound I-19 (4.80 g, 77%).

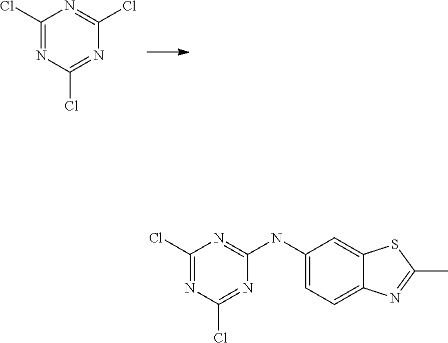

### (4,6-Dichloro-[1,3,5]triazin-2-yl)-(2-methyl-benzothiazol-6-yl)-amine (I-20)

Compound I-20 was prepared according to the procedure for I-3 (2.100 g, 67%).

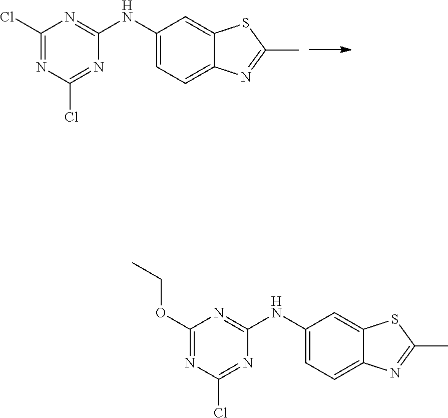

### (4-Chloro-6-ethoxy-[1,3,5]triazin-2-yl)-(2-methyl-benzothiazol-6-yl)-amine (I-21)

Method A A mixture of compound I-19 (500 mg, 2.58 mmol), 2-methyl-benzothiazol-6-ylamine (424 mg, 2.58 mmol), DIPEA (0.500 mL, 2.85 mmol) and THF (10 mL) was stirred at 35° C. for 2 hours, diluted with water and extracted with dichloromethane. The combined organic phases were dried over sodium sulfate, concentrated at reduced pressure and dried to give the compound (749 mg, 90%).

Method B Compound I-21 was prepared according to the procedure for I-2 (160 mg, 50%).

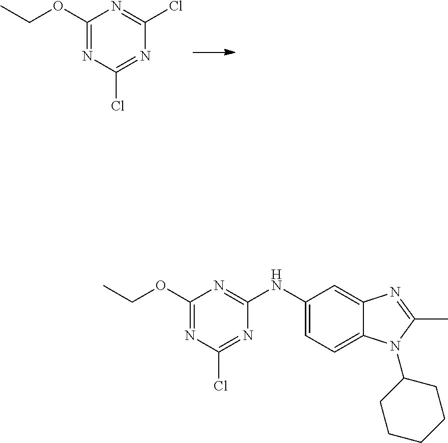

### (4-Chloro-6-ethoxy-[1,3,5]triazin-2-yl)-(1-cyclohexyl-2-methyl-1H-benzoimidazol-5-yl)-amine (I-22)

DIPEA (330 mg, 2.6 mmol) was added dropwise to a mixture of compound I-19 (500 mg, 2.6 mmol), 1-cyclohexyl-2-methyl-1H-benzoimidazol-5-ylamine (780 mg, 2.6 mmol) and THF (10 mL). The obtained mixture was stirred at room temperature for 2 hours, diluted with water. The formed solid was collected by filtration and recrystallized form acetonitrile/water to give I-22 (600 mg, 60%).

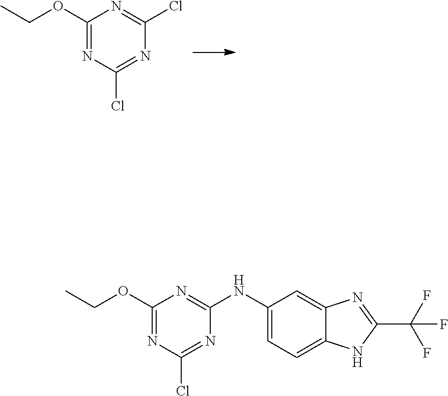

### (4-Chloro-6-ethoxy-[1,3,5]triazin-2-yl)-(2-trifluoromethyl-1H-benzoimidazol-5-yl)-amine (I-23)

Compound I-23 was prepared according to the procedure for I-22 (260 mg, 47%).

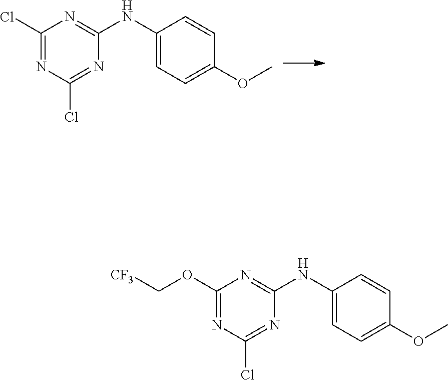

### 2-(4-methoxy-phenylamino)-4-chloro-6-(2,2,2-trifluoroethoxy-[1,3,5]triazine (I-24)

Compound I-24 was prepared according to the procedure for I-2 from I-5 (260 mg, 47%).

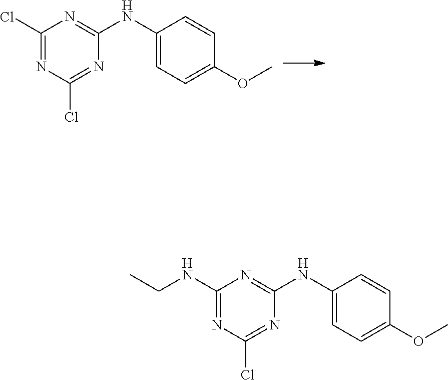

### 6-Chloro-N-ethyl-N′-(4-methoxy-phenyl)-[1,3,5]triazine-2,4-diamine (I-25)

A mixture of compound I-4 (500 mg, 1.84 mmol), ethylamine hydrochloride (144 mg, 1.84 mmol), DIPEA (0.641 mL, 3.68 mmol) and THF (15 mL) was stirred at 50° C. for 11 hours, cooled to room temperature, diluted with water. The formed solid was collected by filtration and washed with methanol to give I-25 (502 mg, 97%).

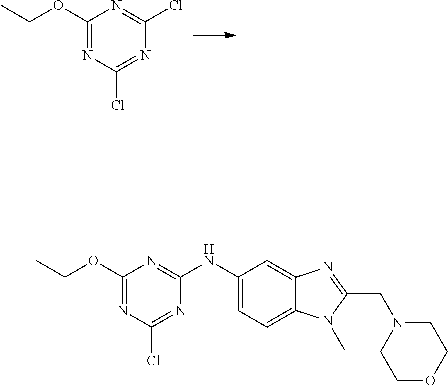

### (4-Chloro-6-ethoxy-[1,3,5]triazin-2-yl)-(1-methyl-2-morpholin-4-ylmethyl-1H-benzoimidazol-5-yl)-amine (I-26)

A mixture of compound I-21 (500 mg, 2.58 mmol), 1-methyl-2-morpholin-4-ylmethyl-1H-benzoimidazol-5-ylamine trihydrochloride (917 mg, 2.58 mmol), DIPEA (1.8 mL0 and THF (12 mL) was stirred at room temperature for 1.5 h, diluted with water and extracted with dichloromethane. The combined organic phases were concentrated at reduced pressure and dried to give I-26 (948 mg, 91%).

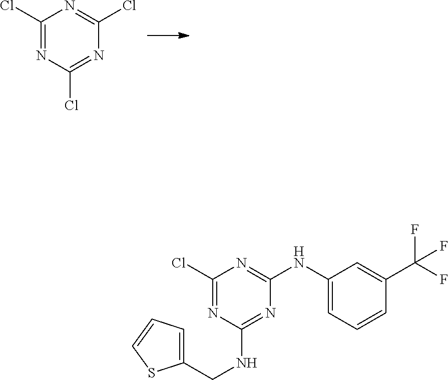

### 6-Chloro-N-thiophen-2-ylmethyl-N′-(3-trifluoromethyl-phenyl)-[1,3,5]triazine-2,4-diamine (I-27)

A solution of amine (1.0 g, 8.84 mmol), DIPEA (1.14 g, 8.84 mmol) in THF (10 mL) was added slowly dropwise to a solution of trichlorotriazine (1.62 g, 8.84 mmol) in THF (10 mL) at −20° C. during 30 min. Then the mixture was stirred at −20° C. for 1.5 hour and warmed up to 0° C. A solution of m-CF₃-aniline (1.42 g, 8.84 mmol) and DIPEA (1.40 g, 8.84 mmol) in THF (10 mL) was added slowly dropwise to the resulting mixture during 30 minutes at 0° C. The obtained solution was stirred at 0° C. for 2 hours, concentrated at reduced pressure, washed with water and extracted with chloroform. The combined organic phases were concentrated at reduced pressure to give desired the compound (3.07 g, 90%) used further without any additional purification.

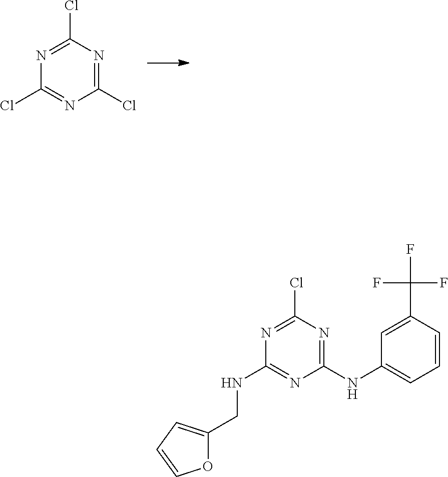

### 6-Chloro-N-furan-2-ylmethyl-N′-(3-trifluoromethyl-phenyl)-[1,3,5]triazine-2,4-diamine (I-28)

Compound I-28 was prepared according to the procedure for I-27 (2.94 g, 90%).

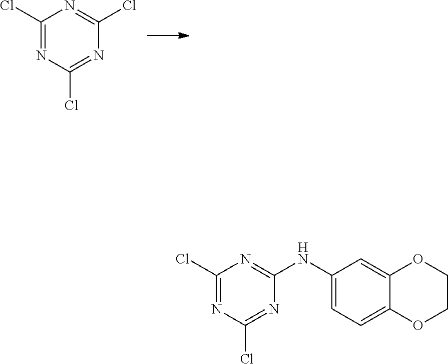

### (4,6-Dichloro-[1,3,5]triazin-2-yl)-(2,3-dihydro-benzo[1,4]dioxin-6-yl)-amine (I-29)

Compound I-29 was prepared according to the procedure for I-3 (4.801 g, 98%).

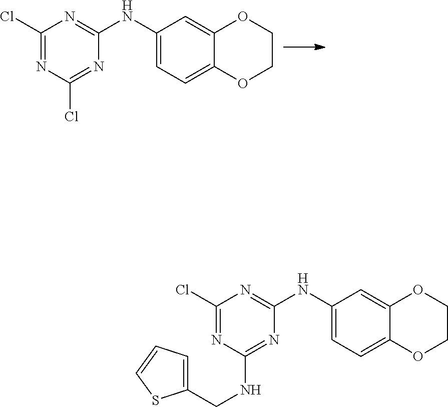

### 6-Chloro-N-(2,3-dihydro-benzo[1,4]dioxin-6-yl)-N′-thiophen-2-ylmethyl-[1,3,5]triazine-2,4-diamine (I-30)

A solution of C-Thiophen-2-yl-methylamine (570 mg, 5 mmol), DIPEA (650 mg, 5 mmol) in THF (25 mL) was added dropwise to a solution of compound I-29 (1.500 g, 5 mmol) in THF (25 mL) at 0° C. The obtained mixture was stirred at 0° C. for 30 minutes, concentrated at reduced pressure and washed with water to give I-30 (1.800 g, 96%).

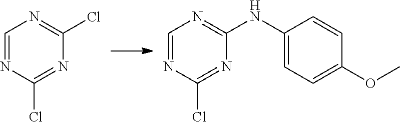

### (4-Chloro-[1,3,5]triazin-2-yl)-(4-methoxy-phenyl)-amine (I-31)

A solution of p-methoxyaniline (246 mg, 2 mmol) and DIPEA (258 mg, 2 mmol) in THF (2.5 mL) was added dropwise to a solution of dichlorotriazine (300 mg, 2 mmol) in THF (2.5 mL) at −5° C. The obtained mixture was allowed to warm up to room temperature and subjected to column chromatography purification (silica gel, ethyl acetate) to give the compound (470 mg, 98%).

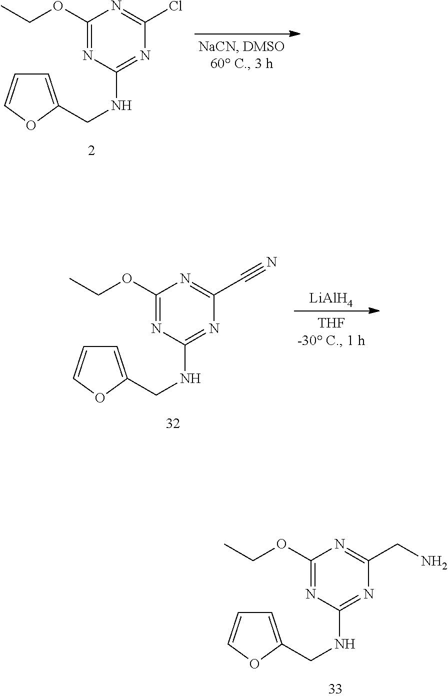

### 4-Ethoxy-6-[(furan-2-ylmethyl)-amino]-[1,3,5]triazine-2-carbonitrile (I-32)

A mixture of compound I-2 (1.200 g, 4.71 mmol), NaCN (1.155 g, 23.56 mmol) and DMSO (12 mL) was stirred at 60° C. for 4 hours, cooled to room temperature, diluted with water and extracted with dichloromethane. The combined organic phases were washed with brine, dried over sodium sulfate and purified by column chromatography (silica gel, dichloromethane) giving the compound (503 mg, 44%).

### (4-Aminomethyl-6-ethoxy-[1,3,5]triazin-2-yl)-furan-2-ylmethyl-amine (I-33)

Compound 32 (500 mg, 2.03 mmol) was added portionwise to a suspension of LiAlH₄ (387 mg, 10.19 mmol) in THF (12 mL) at −35° C. The mixture was stirred at the same temperature for 1 hour and then was let warm up slowly. Ethanol (6 mL) was added dropwise, when internal temperature of the mixture was −10° C. Then 15% aqueous KOH solution (50 mL) was added to the reaction mixture. The formed solid was filtered off and washed with ethyl acetate. The combined solutions were washed with water, brine, dried over sodium sulfate, concentrated at reduced pressure and dried in vacuum giving the compound (329 mg, 65%).

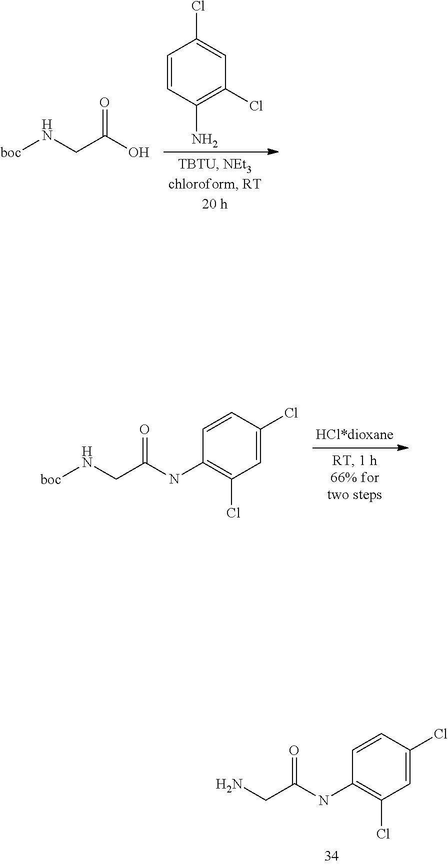

### 2-Amino-N-(2,4-dichloro-phenyl)-acetamide (I-34)

A mixture of N-Boc glycine (410 mg, 2.34 mmol), 2,4-dichloroaniline (316 mg, 1.95 mmol), TBTU (814 mg, 2.53 mmol), NEt₃ (0.73 mL, 5.21 mmol) and chloroform (8 mL) was stirred at room temperature for 20 hours, concentrated at reduced pressure, diluted with saturated aqueous K₂CO₃ solution and extracted with ethyl acetate. The combined organic phases were washed with saturated aqueous NaHCO₃ solution, water and brine and concentrated. The residue was treated with dioxane saturated with HCl (10 mL) at room temperature and the mixture was stirred at room temperature for 1 hour. The formed solid was collected by filtration, washed with dioxane and air-dried giving the compound (240 mg, 66%).

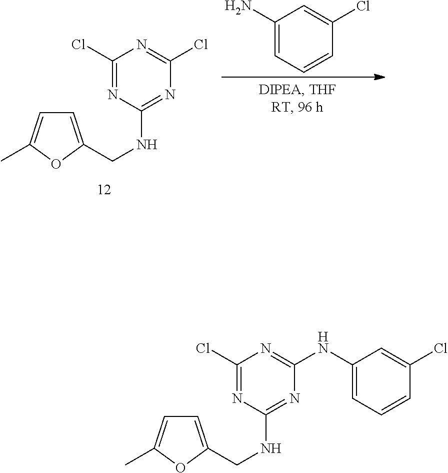

### 6-Chloro-N-(3-chloro-phenyl)-N′-(5-methyl-furan-2-ylmethyl)-[1,3,5]triazine-2,4-diamine (I-35)

A solution of m-chloro aniline (254 mg, 2 mmol) and DIPEA (258 mg, 2 mmol) in THF (10 mL) was added to a solution of compound I-12 (518 mg, 2 mmol) in THF (10 mL). The mixture was stirred at room temperature for 96 hours (TLC control) and washed with water. The aqueous layer was separated and extracted with ethyl acetate. The combined organic phases were dried over sodium sulfate and concentrated at reduced pressure. Purification by column chromatography (silica gel, acetone/dichloromethane) and recrystallization from ethyl acetate/hexane gave the compound. Yield 382 mg, 54%.

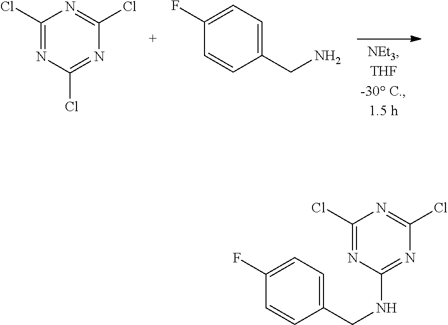

### (4,6-Dichloro-[1,3,5]triazin-2-yl)-(4-fluoro-benzyl)-amine (I-36)

To a solution of cyanuric chloride (20 g, 110 mmol) in THF (100 mL) a solution of p-fluorobenzyl amine (12.5 g, 100 mmol) and NEt₃ (15 mL) in THF (50 mL) was added dropwise at −30° C. during 1.5 hours. The resulting mixture was stirred at −30° C. for 1.5 hours, diluted with water and extracted with dichloromethane. The combined organic phases were dried over sodium sulfate and concentrated. Purification by column chromatography (silica gel, dichloromethane) and triturating with hexane gave the compound. Yield 14.65 g, 54%.

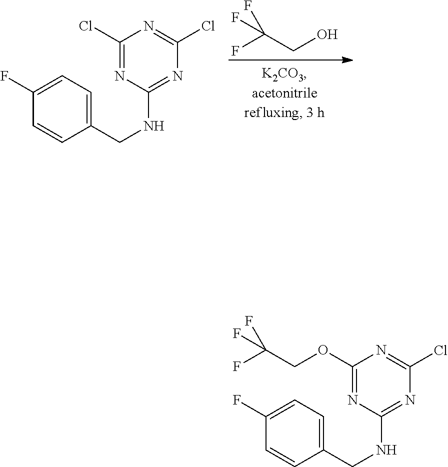

### [4-Chloro-6-(2,2,2-trifluoro-ethoxy)-[1,3,5]triazin-2-yl]-(4-fluoro-benzyl)-amine (I-37)

To a solution of compound 36 (5.0 g, 18.3 mmol) in acetonitrile (40 mL) 2,2,2-trifluoroethanol (2.75 g, 27.5 mmol) and K₂CO₃ (2.53 g, 18.3 mmol) were added. The reaction mixture was stirred under refluxing for 3 hours, cooled to room temperature and concentrated. The residue was triturated with water and diethyl ether and recrystallized from diethyl ether giving the compound. Yield 4.7 g, 76%.

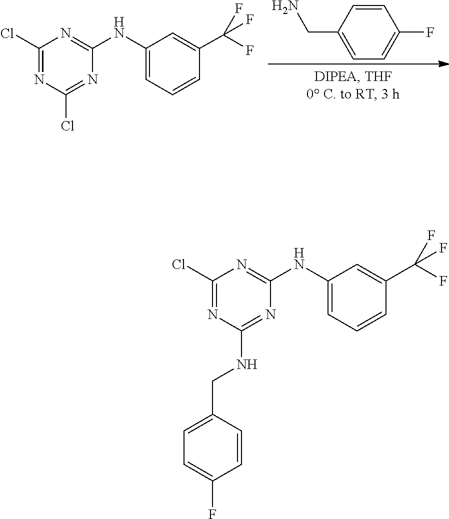

### 6-Chloro-N-(4-fluoro-benzyl)-N′-(3-trifluoromethyl-phenyl)-[1,3,5]triazine-2,4-diamine (I-38)

To a solution of compound 7 (382 mg, 1.236 mmol) in THF (4 mL) a solution of p-fluorobenzylamine (154 mg, 1.236 mmol) and DIPEA (160 mg, 1.236 mmol) in THF (4 mL) was added dropwise at 0° C. The resulting mixture was stirred at 0° C. for 2 hours and at room temperature for 1 hour, concentrated at reduced pressure. The residue was washed with water and a mixture of ethyl acetate/hexane (3/7) and dried giving the compound (397 mg, 81%).

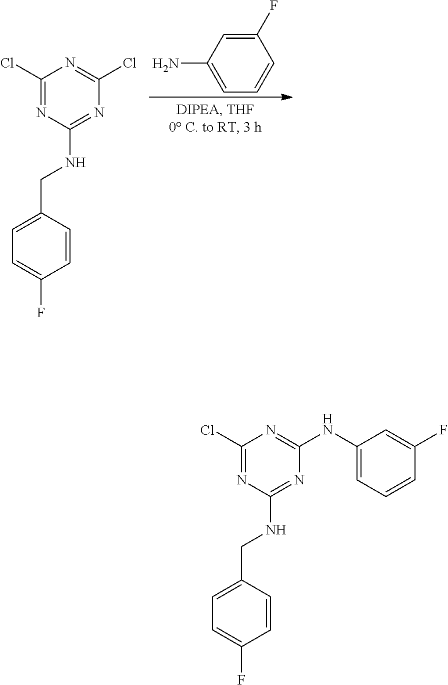

### 6-Chloro-N-(4-fluoro-benzyl)-N′-(3-fluoro-phenyl)-[1,3,5]triazine-2,4-diamine (I-39)

To a solution of compound I-36 (410 mg, 1.5 mmol) in THF (20 mL) a solution of m-fluoroaniline (167, 1.5 mmol) and DIPEA (300 mg, 2.3 mmol) in THF (20 mL) was added at 0° C. The resulting mixture was stirred at 0° C. for 1 hour and at room temperature for 2 hours. The solvent was removed. The residue was diluted with water and extracted with dichloromethane. The combined organic phases were dried over sodium sulfate and concentrated. Purification by column chromatography (silica gel, dichloromethane/hexane) and recrystallization from dichloromethane/hexane) gave the compound (432 mg, 80%).

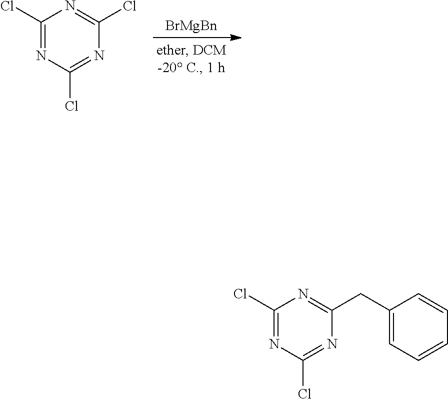

### 2-Benzyl-4,6-dichloro-[1,3,5]triazine (I-40)

To a powder of magnesium (1.022 g, 46.24 mmol) in diethyl ether (15 mL) benzyl bromide (7.190 g, 42.04 mmol) was added dropwise maintaining gentle refluxing of the reaction mixture for 1 hour. refluxing was continued for 1.5 hours, then cooled down to room temperature. Then the obtained solution was added to a solution of cyanuric chloride (7.380 g, 40 mmol) in dichloromethane (50 mL) at −20° C. The resulting mixture was stirred at −20° C. for 1 hour. Water was added dropwise at −10° C. and the organic phase was separated and filtered. The inorganic solid was washed with dichloromethane. The aqueous phase was extracted dichloromethane. the combined organic phases were washed with water and brine, dried over sodium sulfate and concentrated. Purification by column chromatography (silica gel, dichloromethane) gave the compound (8.92 g, 93%).

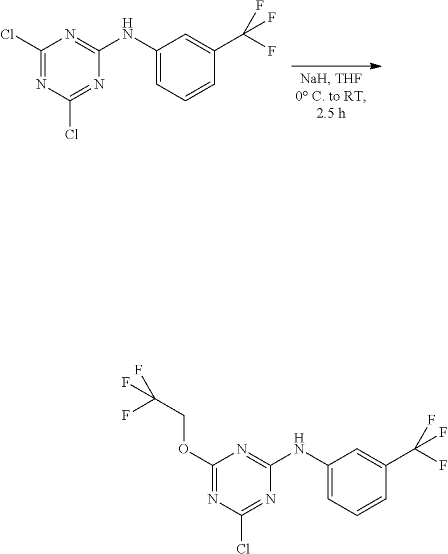

### [4-Chloro-6-(2,2,2-trifluoro-ethoxy)-[1,3,5]triazin-2-yl]-(3-trifluoromethyl-phenyl)-amine (I-41)

Sodium hydride (60% in oil, 260 mg, 6.48 mmol) was added to a solution of 2,2,2-trifluoroethanol (650 mg, 6.48 mmol) in THF (25 mL) at room temperature. The obtained solution was stirred at the same temperature for 30 minutes. Then it was added dropwise to a solution of compound 7 (2.00 g, 6.48 mmol) in THF (25 mL) at 0° C. The resulting mixture was stirred at 0° C. for 1.5 hour and at room temperature for 1 hour, diluted with water and extracted with dichloromethane (2×70 mL). The combined organic phases were dried over potassium carbonate and concentrated. The residue was triturated with hexane and recrystallized (hexane/diethyl ether, 5H) giving 1.45 g of the compound with purity 75% (LCMS). The compound was used on the next stage without additional purification. MW 372.66. LCMS t_{R} (min): 2.03. MS (APCI+), m/z 373, 375 [M+H]⁺.

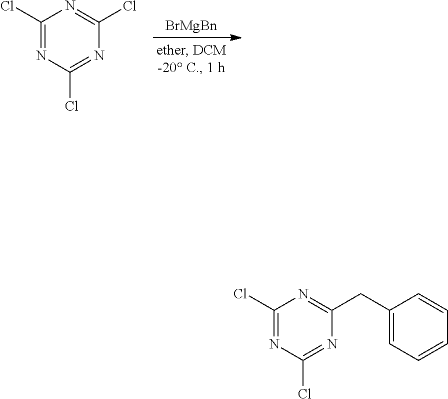

### 2-Benzyl-4,6-dichloro-[1,3,5]triazine (I-42)

To a powder of magnesium (1.022 g, 46.24 mmol) in diethyl ether (15 mL) benzyl bromide (7.190 g, 42.04 mmol) was added dropwise maintaining gentle refluxing of the reaction mixture for 1 hour. refluxing was continued for 1.5 hours, then cooled down to room temperature. Then the obtained solution was added to a solution of cyanuric chloride (7.380 g, 40 mmol) in dichloromethane (50 mL) at −20° C. The resulting mixture was stirred at −20° C. for 1 hour. Water was added dropwise at −10° C. and the organic phase was separated and filtered. The inorganic solid was washed with dichloromethane. The aqueous phase was extracted dichloromethane. the combined organic phases were washed with water and brine, dried over sodium sulfate and concentrated. Purification by column chromatography (silica gel, dichloromethane) gave the compound. Yield 8.92 g, 93%.

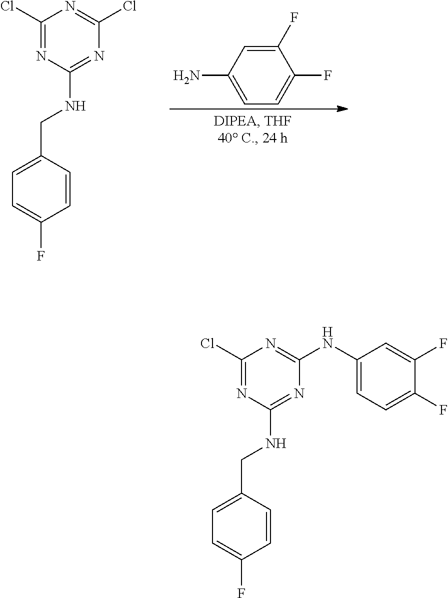

### 6-Chloro-N-(3,4-difluoro-phenyl)-N′-(4-fluoro-benzyl)-[1,3,5]triazine-2,4-diamine (I-43)

To a solution of compound I-36 (891 mg, 3 mmol) in THF (5 mL) a solution of 3,4-difluoro-phenylamine (387 mg, 3 mmol) and DIPEA (387 mg, 3 mmol) in THF (5 mL) was added dropwise. The reaction mixture was stirred at 40° C. for 24 hours, cooled to room temperature, diluted with ethyl acetate and diethyl ether, washed with water, concentrated, dissolved in dichloromethane and purified by column chromatography (silica gel, dichloromethane) giving the compound. Yield 920 mg, 84%.

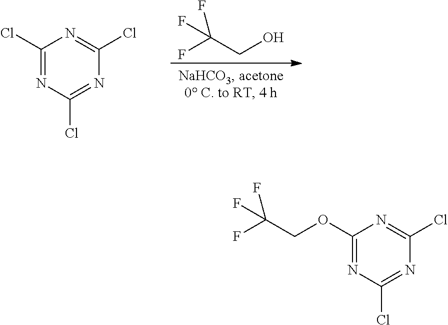

### 2,4-Dichloro-6-(2,2,2-trifluoro-ethoxy)-[1,3,5]triazine (I-44)

A mixture of cyanuric chloride (5.532 g, 30 mmol), 2,2,2-trifluoroethanol (3.000 g, 30 mmol), NaHCO₃ (2.520 g, 30 mmol) and acetone (15 mL) was stirred at 0° C. for 4 hours slowly warming up to room temperature, then diluted with water and extracted with dichloromethane. The combined organic phases were concentrated at reduced pressure. The residue was purified by column chromatography (silica gel, ethyl acetate/hexane) to remove an excess of cyanuric chloride. The rest amount of cyanuric chloride was isolated by crystallization from I-44 giving the desired compound.

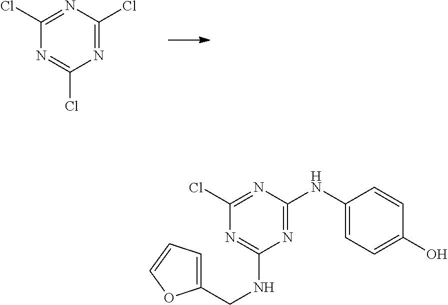

### 2-(4-Hydroxy-phenylamino)-4-Chloro-6-[(furan-2-ylmethyl)-amino-]-[1,3,5]triazine (I-45)

To a cooled to −20° C. solution of cyanuric chloride (18.44 g, 100 mmol) in anhydrous THF (200 mL) a solution of N,N-diisopropylethylamine (13.57 g, 105 mmol) and furfurylamine (9.71 g, 100 mmol) in anhydrous THF (150 mL) was added dropwise at −20° C. for a period of 30 minutes. The resulting mixture was stirred at −20° C. for 1 hour (TLC control). Then the reaction mixture was allowed to warm up to 0° C. A solution of N,N-diisopropylethylamine (13.57 g, 105 mmol) and p-aminophenol (10.91 g, 100 mmol) in anhydrous THF (200 mL) was added dropwise at 0° C. to the reaction mixture. The resulting mixture was stirred at 0° C. for 2 hours then at room temperature for 10 hours. The solvent was removed at reduced pressure. The crude product was purified by column chromatography on silica gel (methanol/ethyl acetate/hexane) giving I-45 (13.66 g, 43%).

### Section 2. Preparation of R2-Triazine Libraries

### Prepare N,N,N-triazines in Table 5

The “R2-variation” compounds containing N-linker at 2-position were synthesized through three step reaction sequence as shown in Schemes 1 and 2:

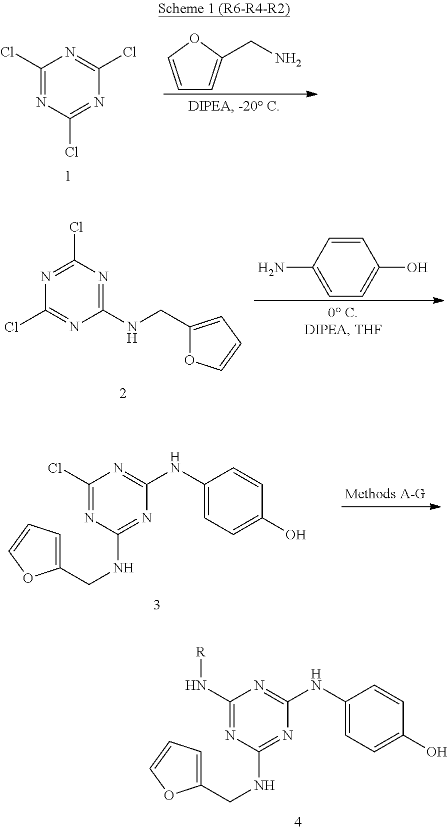

The general method used for the synthesis of a series of N,N,N-substituted triazines is to prepare a key intermediate 3 via two step “one pot” sequence. A mixture of compound 3, the corresponding amine, and base listed in the following table in the minimal volume of DMSO were heated up to 200° C., following standard column chromatography in combination with preparative HPLC method to provide final compounds 4.

| Method | Conditions |
|---|---|
| A | K₂CO₃, min. volume DMSO, 150-200^{° C.}, 2-3 h. |
| B | Pd₃(dba)₂, PBu^{t}₃, toluene, NaOBu^{t}, refluxing, 1-4 h |
| C | DIPEA, 100^{° C.}, 2 hours |
| D | DIPEA, DMSO, 100^{° C.} |
| E | K₂CO₃, DMF, 150^{° C.} |
| F | fusion, 120-200^{° C.}, 1 h |
| G | NEt₃, ethanol, refluxing, 1 h |

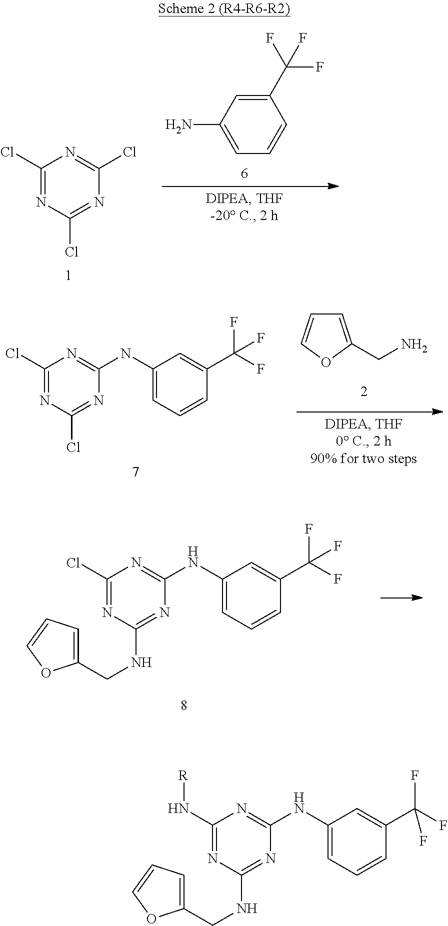

A compound containing biarylic moiety at 2-position was synthesized in 4 steps as shown in Scheme 3:

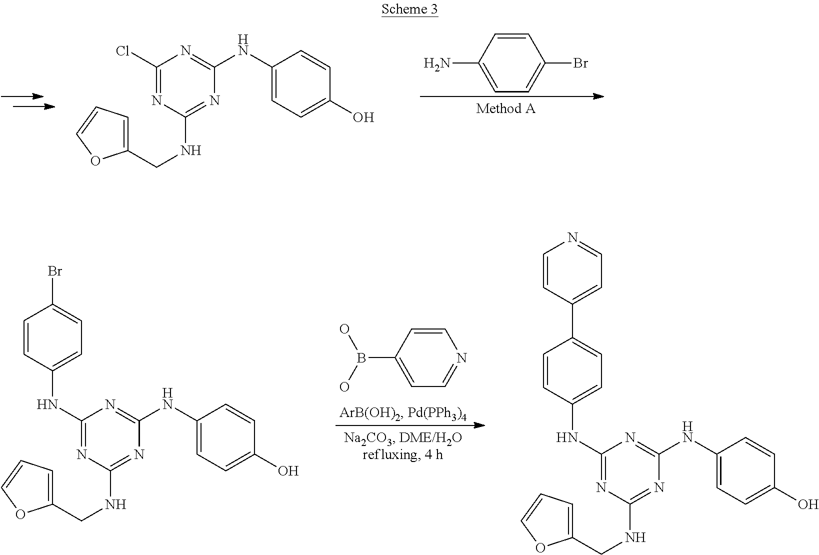

TABLE 5

| En | Structure | IUPAC Name | MW | Formula |
|---|---|---|---|---|
| 1 | 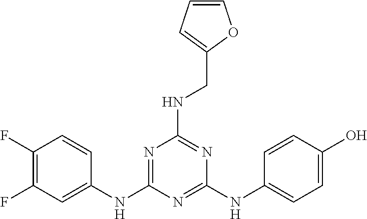 | 4-({4-[(3,4-difluorophenyl)amino]- 6-[(furan-2-ylmethyl)amino]-1,3,5- triazin-2-yl}amino)phenol | 410.4 | C20H16F2N6O2 |
| 2 | 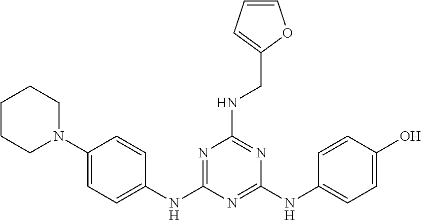 | 4-[(4-[(furan-2-ylmethyl)amino]-6- {[4-(piperidin-1-yl)phenyl]amino}- 1,3,5-triazin-2-yl)amino]phenol | 457.5 | C25H27N7O2 |
| 3 | 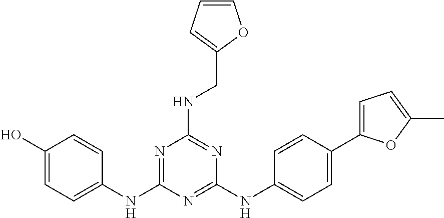 | 4-[(4-[(furan-2-ylmethyl)amino]-6- {[4-(5-methylfuran-2- yl)phenyl]amino}-1,3,5-triazin-2- yl)amino]phenol | 454.5 | C25H22N6O3 |
| 4 | 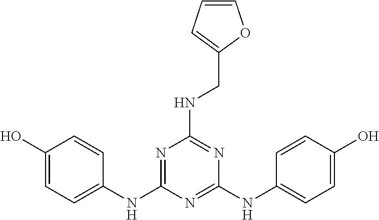 | 4,4″-({6-[(furan-2- ylmethyl)amino]-1,3,5-triazine- 2,4-diyl}diimino)diphenol | 390.4 | C20H18N6O3 |
| 5 | 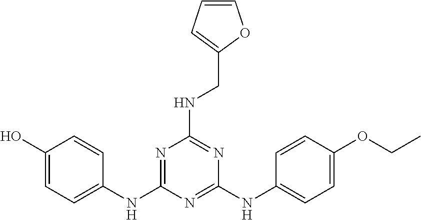 | 4-({4-[(4-ethoxyphenyl)amino]-6- [(furan-2-ylmethyl)amino]-1,3,5- triazin-2-yl}amino)phenol | 418.5 | C22H22N6O3 |
| 6 | 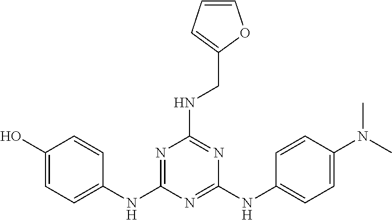 | 4-[(4-{[4- (dimethylamino)phenyl]amino}-6- [(furan-2-ylmethyl)amino]-1,3,5- triazin-2-yl)amino]phenol hydrochloride | 417.5 | C22H24ClN7O2 |
| 7 | 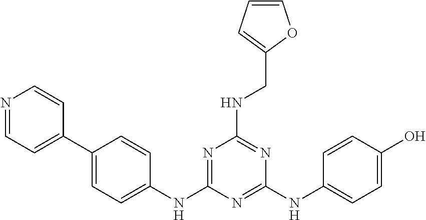 | 4-[(4-[(furan-2-ylmethyl)amino]-6- {[4-(pyridin-4-yl)phenyl]amino}- 1,3,5-triazin-2-yl)amino]phenol | 451.5 | C25H21N7O2 |
| 8 | 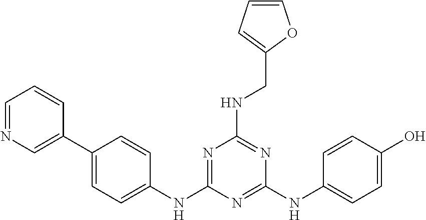 | 4-[(4-(furan-2-ylmethyl)amino]-6- {[4-(pyridin-3-yl)phenyl]amino}- 1,3,5-triazin-2-yl)amino]phenol | 451.5 | C25H21N7O2 |
| 9 | 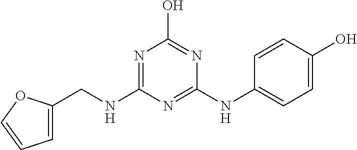 | 4-[(furan-2-ylmethyl)amino]-6-[(4- hydroxyphenyl)amino]-1,3,5- triazin-2-ol | 299.3 | C14H13N5O3 |
| 10 | 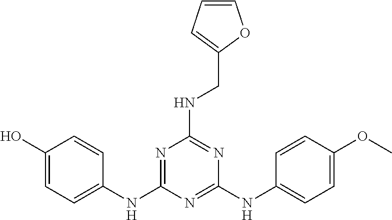 | 4-({4-[(furan-2-ylmethyl)amino]-6- [(4-methoxyphenyl)amino]-1,3,5- triazin-2-yl}amino)phenol | 404.4 | C21H20N6O3 |
| 11 | 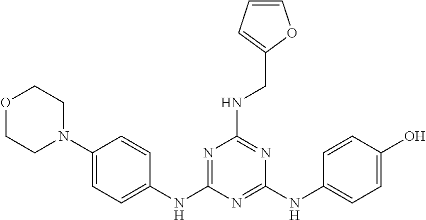 | 4-[(4-[(furan-2-ylmethyl)amino]-6- {[4-(morpholin-4-yl)phenyl]amino}- 1,3,5-triazin-2-yl)amino]phenol | 459.5 | C24H25N7O3 |
| 12 | 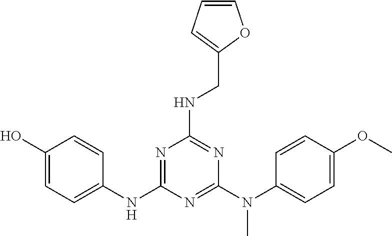 | 4-({4-[(furan-2-ylmethyl)amino]-6- [(4-methoxyphenyl)(methyl)amino]- 1,3,5-triazin-2-yl}amino)phenol | 418.5 | C22H22N6O3 |
| 13 | 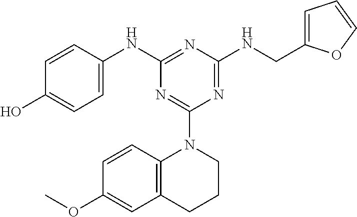 | 4-({4-[(furan-2-ylmethyl)amino]-6-(6- methoxy-3,4-dihydroquinolin-1(2H)- yl)-1,3,5-triazin-2-yl}amino)phenol | 444.5 | C24H24N6O3 |
| 14 | 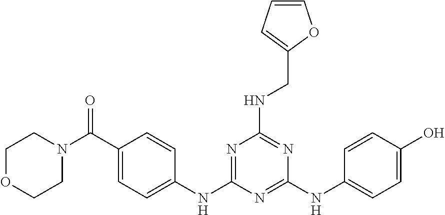 | [4-({4-[(furan-2-ylmethyl)amino]-6- [(4-hydroxyphenyl)amino]-1,3,5- triazin-2-yl}amino)phenyl](morpholin- 4-yl)methanone | 487.5 | C25H25N7O4 |
| 15 | 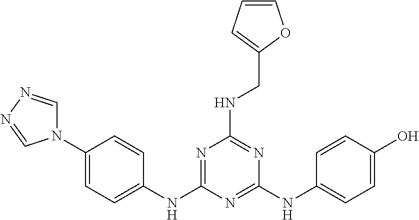 | 4-[(4-[(furan-2-ylmethyl)amino]-6-{[4- (4H-1,2,4-triazol-4-yl)phenyl]amino}- 1,3,5-triazin-2-yl)amino]phenol | 441.4 | C22H19N9O2 |
| 16 | 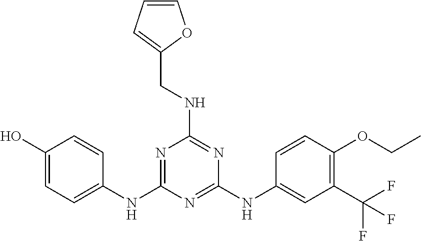 | 4-[(4-{[4-ethoxy-3- (trifluoromethyl)phenyl]amino}-6- [(furan-2-ylmethyl)amino]-1,3,5- triazin-2-yl)amino]phenol | 486.4 | C23H21F3N6O3 |
| 17 | 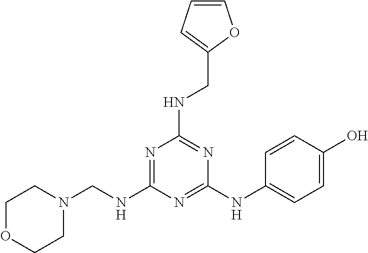 | 4-({4-[(furan-2-ylmethyl)amino]-6- (morpholin-4-yl)-1,3,5-triazin-2- yl}amino)phenol | 368.4 | C18H20N6O3 |
| 18 | 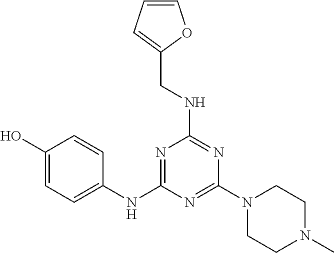 | 4-({4-[(furan-2-ylmethyl)amino]-6- (4-methylpiperazin-1-yl)-1,3,5- triazin-2-yl}amino)phenol | 381.4 | C19H23N7O2 |
| 19 | 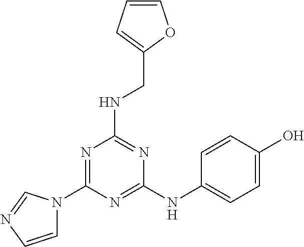 | 4-({4-[(furan-2-ylmethyl)amino]-6- (1H-imidazol-1-yl)-1,3,5-triazin-2- yl}amino)phenol | 349.3 | C17H15N7O2 |
| 20 | 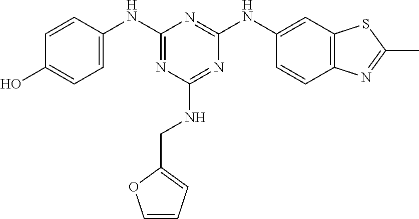 | 4-({4-[(furan-2-ylmethyl)amino]-6-[(2- methyl-1,3-benzothiazol-6-yl)amino]- 1,3,5-triazin-2-yl}amino)phenol | 445.5 | C22H19N7O2S |
| 21 | 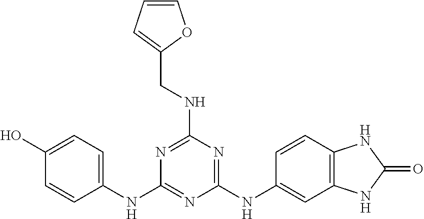 | 5-({4-[(furan-2-ylmethyl)amino]-6- [(4-hydroxy-phenyl)amino]-1,3,5- triazin-2-yl}amino)-1,3-dihydro-2H- benzimidazol-2-one | 430.4 | C21H18N8O3 |
| 22 | 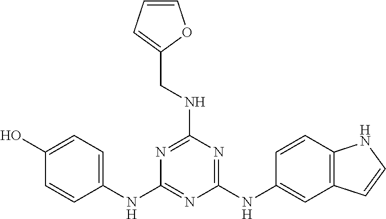 | 4-({4-[(furan-2-ylmethyl)amino]-6- (1H-indol-5-ylamino)-1,3,5-triazin- 2-yl}amino)phenol | 413.4 | C22H19N7O2 |
| 23 | 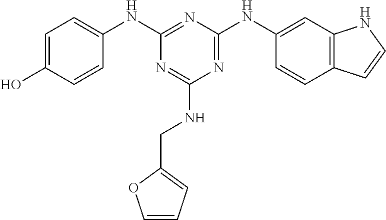 | 4-({4-[(furan-2-ylmethyl)amino]-6- (1H-indol-6-ylamino)-1,3,5-triazin- 2-yl}amino)phenol | 413.4 | C22H19N7O2 |
| 24 | 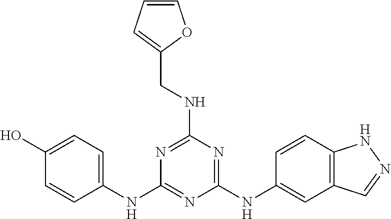 | 4-({4-[(furan-2-ylmethyl)amino]-6- (1H-indazol-5-ylamino)-1,3,5-triazin- 2-yl}amino)phenol | 414.4 | C21H18N8O2 |
| 25 | 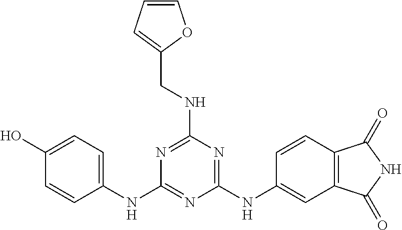 | 5-({4-[(furan-2-ylmethyl)amino]-6- [(4-hydroxyphenyl)amino]-1,3,5- triazin-2-yl}amino)-1H-isoindole- 1,3(2H)-dione | 443.4 | C22H17N7O4 |
| 26 | 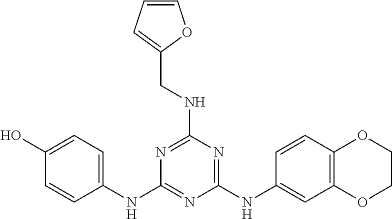 | 4-({4-(2,3-dihydro-1,4-benzodioxin-6- ylamino)-6-[(furan-2-ylmethyl)amino]- 1,3,5-triazin-2-yl}amino)phenol | 432.4 | C22H20N6O4 |
| 27 | 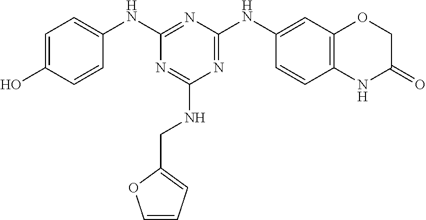 | 7-({4-[(furan-2-ylmethyl)amino]-6- [(4-hydroxypheny)amino]-1,3,5- triazin-2-yl}amino)-2H-1,4- benzoxazin-3(4H)-one | 445.4 | C22H19N7O4 |
| 28 | 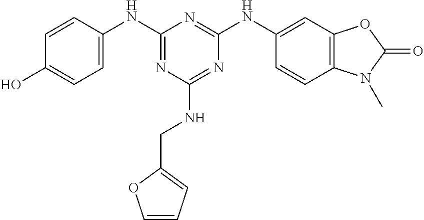 | 6-({4-[(furan-2-ylmethyl)amino]-6- [(4-hydroxyphenyl)amino]-1,3,5- triazin-2-yl}amino)-3-methyl-1,3- benzoxazol-2(3H)-one | 445.4 | C22H19N7O4 |
| 29 | 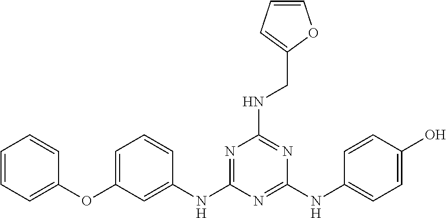 | 4-({4-[(furan-2-ylmethyl)amino]-6- [(3-phenoxyphenyl)amino]-1,3,5- triazin-2-yl}amino)phenol | 466.5 | C26H22N6O3 |
| 30 | 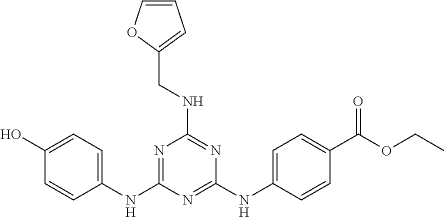 | ethyl 4-({4-[(furan-2-ylmethyl)amino]- 6-[(4-hydroxyphenyl)amino]-1,3,5- triazin-2-yl}amino)benzoate | 446.5 | C23H22N6O4 |
| 31 | 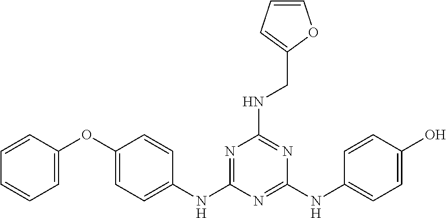 | 4-({4-[(furan-2-ylmethyl)amino]-6- [(4-phenoxyphenyl)amino]-1,3,5- triazin-2-yl}amino)phenol | 466.5 | C26H22N6O3 |
| 32 | 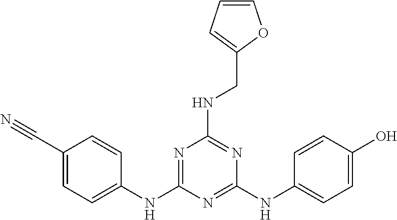 | 4-({4-[(furan-2-ylmethyl)amino]-6- [(4-hydroxyphenyl)amino]-1,3,5- triazin-2-yl}amino)benzonitrile | 399.4 | C21H17N7O2 |
| 33 | 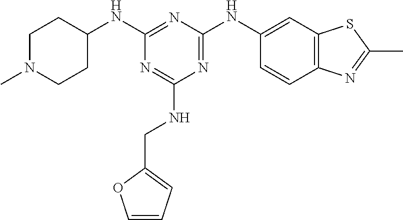 | N~2~-(furan-2-ylmethyl)-N~4~-(2- methyl-1,3-benzothiazol-6-yl)- N~6~-(1-methylpiperidin-4-yl)- 1,3,5-triazine-2,4,6-triamine | 450.6 | C22H26N8OS |
| 34 | 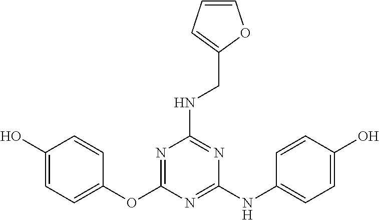 | 4-({4-[(furan-2-ylmethyl)amino]-6- (4-hydroxy-phenoxy)-1,3,5-triazin- 2-yl}amino)phenol | 391.4 | C20H17N5O4 |
| 35 | 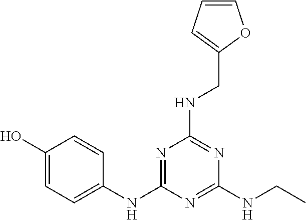 | 4-({4-(ethylamino)-6-[(furan-2- ylmethyl)-amino]-1,3,5-triazin-2- yl}amino)phenol | 326.4 | C16H18N6O2 |
| 36 | 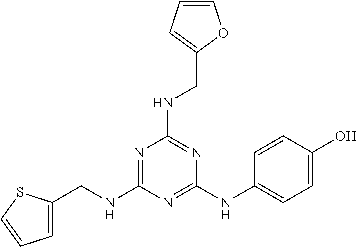 | 4-({4-[(furan-2-ylmethyl)amino]-6- [(thiophen-2-ylmethyl)amino]-1,3,5- triazin-2-yl}amino)phenol | 394.5 | C19H18N6O2S |
| 37 | 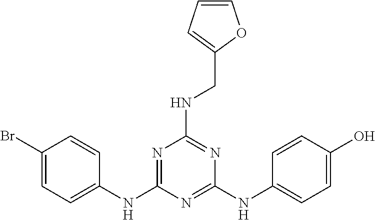 | 4-({4-[(4-bromophenyl)amino]-6- [(furan-2-ylmethyl)amino]-1,3,5- triazin-2-yl}amino)phenol | 453.3 | C20H17BrN6O2 |
| 38 | 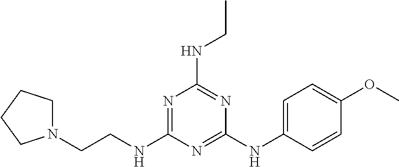 | N2-ethyl-N4-(4-methoxyphenyl)-N6 [symbol] [2-(pyrrolidin-1-yl)ethyl]-1,3,5- triazine-2,4,6-triamine hydrochlorid [symbol] | 357.5 | C18H28ClN7O |
| 39 | 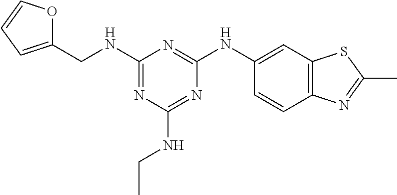 | N~2~-ethyl-N~4~-(furan-2-ylmethyl)- N~6~-(2-methyl-1,3-benzothiazol- 6-yl)-1,3,5-triazine-2,4,6-triamine | 381.5 | C18H19N7OS |
| 40 | 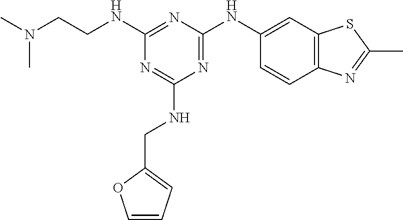 | N~2~-[2-(dimethylamino)ethyl]- N~4~-(furan-2-ylmethyl)-N~6~-(2- methyl-1,3-benzothiazol-6-yl)-1,3,5- triazine-2,4,6-triamine | 424.5 | C20H24N8OS |
| 41 | 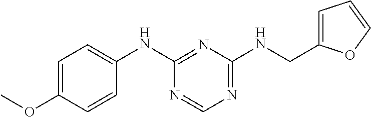 | N-(furan-2-ylmethyl)-N′-(4- methoxyphenyl)-1,3,5-triazine- 2,4-diamine | 297.3 | C15H15N5O2 |
| 42 | 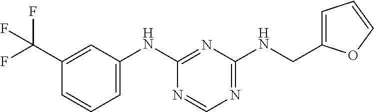 | N-(furan-2-ylmethyl)-N′-[3- (trifluoromethyl)-phenyl]-1,3,5- triazine-2,4-diamine | 335.3 | C15H12F3N5O |
| 43 | 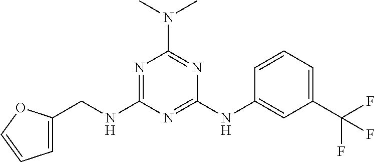 | N~4~-(furan-2-ylmethyl)-N~2~, N~2~-dimethyl-N~6~-[3- (trifluoromethyl)phenyl]-1,3,5- triazine-2,4,6-triamine | 378.4 | C17H17F3N6O |
| 44 | 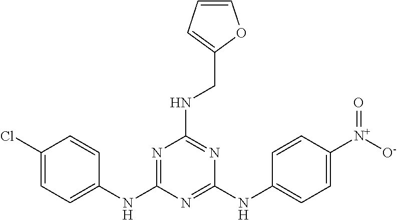 | N~2~-(4-chlorophenyl)-N~4~-(furan- 2-ylmethyl)-N~6~-(4-nitrophenyl)- 1,3,5-triazine-2,4,6-triamine | 437.9 | C20H16ClN7O3 |
| 45 | 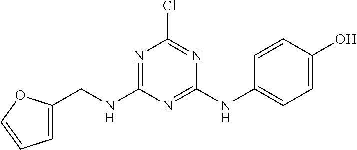 | 4-({4-chloro-6-[(furan-2- ylmethyl)amino]-1,3,5-triazin-2- yl}amino)phenol | 317.7 | C14H12ClN5O2 |
| 46 | 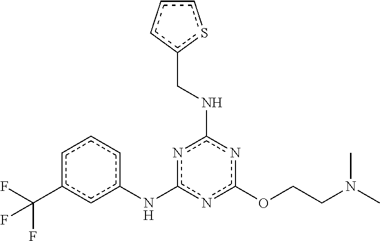 | 6-[2-(dimethylamino)ethoxy]-N- (thiophen-2-ylmethyl)-N′-[3- (trifluoromethyl)phenyl]-1,3,5- triazine-2,4-diamine | 438.5 | C19H21F3N6OS |
| 47 | 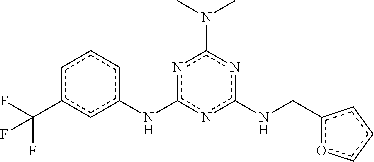 | N~4~-(furan-2-ylmethyl)- N~2~,N~2~-dimethyl-N~6~-[3- (trifluoromethyl)phenyl]-1,3,5- triazine-2,4,6-triamine | 378.4 | C17H17F3N6O |

[symbol]

indicates data missing or illegible when filed

### Procedures and Analytical Data for Compounds in Table 5.

### 1. 4-{4-(3,4-Difluoro-phenylamino)-6-[(furan-2-ylmethyl)-amino]-[1,3,5]triazin-2-ylamino}-phenol

A mixture of compound 45 (317 mg, 1 mmol), 3,4-difluoroaniline (129 mg, 1 mmol), anhydrous K₂CO₃ (415 mg, 3 mmol) and DMF (200 μL) was heated at 150° C. for 2 hours. After cooling to room temperature the resulting mixture was washed with hot water (50 mL) and extracted with hot ethyl acetate (2×30 mL). The combined organic phases were dried over Na₂SO₄ and concentrated. Purification by column chromatography on silica gel (dichloromethane) gave ASE the product as crystals (29 mg, 7%).

¹H-NMR (400 MHz, DMSO-D₆) δ_{H}: 4.46 (2H, d, J=7.4 Hz), 6.23 (1H, broad), 6.37 (1H, broad), 6.67 (2H, d, J=8.5 Hz), 7.15-7.30 (1H, m), 7.35-7.50 (4H, m), 7.53 (1H, d, J=1.8 Hz), 8.07 (1H, broad), 8.77 (1H, broad), 8.95 (1H, broad), 9.12 (1H, broad). LCMS t_{R} 2.34 (min). MS (APCI), m/z 410.99 [M+H]⁺. Mₚ 75-77° C.

### 2. 4-[4-[(Furan-2-ylmethyl)-amino]-6-(4-piperidin-1-yl-phenylamino)-[1,3,5]triazin-2-ylamino]-phenol

¹H-NMR (400 MHz, DMSO-D₆) δ_{H}: 1.49-1.55 (2H, m), 1.55-1.68 (4H, m), 2.97-3.08 (4H, m4), 4.47 (2H, d, J=7.5 Hz), 6.22 (1H, broad, Z/E forms), 6.36 (1H, broad, Z/E forms), 6.64 (2H, d, J=8.5 Hz), 6.81 (2H, d, J=8.5 Hz), 7.14 (1H, broad), 7.46 (2H, d, J=8.5 Hz), 7.48-7.55 (3H, m), 8.60 (2H, broad, Z/E forms), 8.89 (1H, s). LCMS t_{R} 1.88 (min). MS (APCI), m/z 458.16 [M+H]⁺. Mₚ 51-53° C.

### 3. 4-{4-[(Furan-2-ylmethyl)-amino]-6-[4-(5-methyl-furan-2-yl)-phenylamino]-[1,3,5]triazin-2-ylamino}-phenol

¹H-NMR (400 MHz, DMSO-D₆) δ_{H}: 2.31 (3H, s), 4.40 (2H, d, J=7.5 Hz), 6.17 (1H, d, J=3.6 Hz), 6.23 (1H, broad), 6.37 (1H, broad), 6.62 (1H, d, J=3.6 Hz), 6.68 (2H, d, J=8.5 Hz), 7.28 (1H, broad), 7.46 (4H, d, J=8.5 Hz), 7.53 (1H, d, J=1.8 Hz), 7.78 (2H, d, J=8.5 Hz), 8.68 (1H, broad), 8.92 (1H, s), 8.98 (1H, broad). LCMS t_{R} 2.57 (min). MS (APCI), m/z 455.05 [M+H]⁺. Mₚ 62-64° C.

### 4. 4,6-bis-(4-methoxy-phenylamino)-[1,3,5]triazin-2-ylamino]-phenol

¹H-NMR (400 MHz, DMSO-D₆) δ_{H}: 4.46 (2H, d, J=7.5 Hz), 6.22 (1H, broad, Z/E forms), 6.36 (1H, broad, Z/E forms), 6.64 (4H, d, J=8.5 Hz), 7.24 (1H, broad, Z/E forms), 7.43 (4H, d, J=8.5 Hz), 7.53 (1H, s), 8.68 (2H, broad, Z/E forms), 8.92 (1H, s). LCMS t_{R} 1.39 (min). MS (APCI), m/z 390.75 [M+H]⁺. Mₚ 44-46° C.

### 5. 2-(4-Hydroxy-phenylamino)-4-(4-ethoxy-phenylamino)-6-[(furan-2-ylmethyl)-amino]-[1,3,5]triazine

A mixture of compound 45 (317 mg, 1 mmol), p-ethoxyaniline (135 mg, 1 mmol), anhydrous K₂CO₃ (415 mg, 3 mmol) and DMSO (200 μL) was heated at 200° C. for 5 minutes. After cooling to room temperature the resulting mixture was washed with hot water (50 mL) and extracted with hot ethyl acetate (2×30 mL). The combined organic phases were dried over Na₂SO₄ and concentrated. Purification by column chromatography on silica gel (dichloromethane) furnished the product (115 mg, 28%).

¹H-NMR (400 MHz, DMSO-D₆) δ_{H}: 1.31 (3H, t, J=7.5 Hz), 3.98 (2H, q, J=7.5 Hz), 4.47 (2H, d, J=7.5 Hz), 6.23 (1H, dd, J=3.6, 1.8 Hz), 6.36 (1H, d, J=3.6 Hz), 6.63 (2H, d, J=8.5 Hz), 8.78 (2H, d, J=8.5 Hz), 7.25 (1H, broad), 7.44 (2H, d, J=8.5 Hz), 7.51 (1H, d, J=1.8 Hz), 7.58 (2H, d, J=8.5 Hz), 8.53-8.80 (2H, broad, Z/E forms), 8.88 (1H, s). LCMS t_{R} (min) 2.25. MS (APCI), m/z 419.06 [M+H]⁺. Mₚ 43-45° C.

### 6. 4-{4-(4-Dimethylamino-phenylamino)-6-[(furan-2-ylmethyl)-amino]-[1,3,5]triazin-2-ylamino}-phenol

¹H-NMR (400 MHz, DMSO-D₆) δ_{H}: 2.83 (6H, s), 4.47 (2H, d, J=7.5 Hz), 6.22 (1H, broad, Z/E forms), 6.36 (1H, broad, Z/E forms), 6.60-6.68 (4H, m), 7.12 (1H, broad), 7.42-7.50 (4H, m), 7.53 (1H, d, J=8.5 Hz), 8.55 (2H, broad, Z/E forms), 8.89 (1H, s). LCMS t_{R} 1.87 (min). MS (APCI), m/z 418.06 [M+H]⁺. Mₚ 62-64° C.

### 7. 4-[4-[(Furan-2-ylmethyl)-amino]-6-(4-pyridin-4-yl-phenylamino)-[1,3,5]triazin-2-ylamino]-phenol

A mixture of compound 37 (453 mg, 1.0 mmol), 4-pyridyl boronic acid (123 mg, 1.0 mmol), Pd(PPh₃)₄ (57 mg, 0.05 mol), Na₂CO₃ (430 mg, 4.0 mmol), dimethoxy ethane (2 mL) and water (2 mL) was stirred at refluxing for 4 hours, cooled to room temperature, diluted with water (20 mL) and extracted with ethyl acetate (2×20 mL). The combined organic phases were combined, dried over sodium sulfate and concentrated. Purification by column chromatography (silica gel, methanol/ethyl acetate) and preparative TLC gave the compound as yellow solid (27 mg, 6%).

¹H-NMR (400 MHz, DMSO-D₆) δ_{H}: 4.52 (2H, broad), 6.26 (1H, broad), 6.38 (1H, d, J=3.6 Hz), 6.70 (2H, d, J=8.5 Hz), 7.32 (1H, broad), 7.48 (2H, d, J=8.5 Hz), 7.55 (1H, d, J=1.8 Hz), 7.66 (2H, d, J=5.0 Hz), 7.68 (2H, d, J=8.5 Hz), 7.94 (2H, d, J=8.5 Hz), 8.58 (2H, d, J=8.5 Hz), 8.75 (1H, broad), 8.92 (1H, s), 9.15 (1H, broad). LCMS t_{R} 1.35 (min). MS (APCI), m/z 452.02 [M+H]⁺.

### 8. 4-[4-[(Furan-2-ylmethyl)-amino]-6-(4-pyridin-3-yl-phenylamino)-[1,3,5]triazin-2-ylamino]-phenol

A mixture of compound 37 (679 mg, 1.5 mmol), 3-pyridyl boronic acid (184 mg, 1.5 mmol), Pd(PPh₃)₄ (173 mg, 0.15 mol, 10 mol %), Na₂CO₃ (430 mg, 4.0 mmol), dimethoxy ethane (2 mL) and water (2 mL) was stirred at 60° C. for 4 hours, cooled to room temperature, diluted with water (20 mL) and extracted with dichloromethane. The combined organic phases were dried over sodium sulfate and concentrated. Purification by column chromatography (silica gel, dichloromethane) and preparative HPLC (acetonitrile/water) gave the compound.

Yield 30 mg, 7%. ¹H-NMR (400 MHz, DMSO-D₆) δ_{H}: 4.52 (2H, broad), 6.28 (1H, broad), 6.39 (1H, broad), 6.69 (2H, d, J=8.5 Hz), 7.32 (1H, broad, Z/E forms), 7.42-7.52 (3H, m), 7.55 (1H, s), 7.60 (2H, d, J=8.5 Hz), 7.92 (2H, d, J=8.5 Hz), 8.03 (1H, d, J=8.5 Hz), 8.52 (1H, d, J=5.0 Hz), 8.73 (1H, broad), 8.87 (1H, s), 8.92 (1H, s), 9.08 (1H, broad). LCMS t_{R} 1.40 (min). MS (APCI), m/z 452.11 [M+H]⁺. HPLC t_{R} (min): 8.18. Mₚ 110-112° C.

### 9. 4-[(Furan-2-ylmethyl)-amino]-6-(4-hydroxy-phenylamino)-[1,3,5]-triazin-2-ol

A mixture of compound 45 (320 mg, 1 mmol), sodium hydroxide (0.12 g, 3 mmol) and water (10 mL) was stirred at refluxing for 4 hours, cooled to room temperature and neutralized with concentrated HCl aqueous solution to reach pH 2. The formed solid was collected by filtration, washed with water and cold ethanol to give the compound (105 mg, 35%).

¹H-NMR (400 MHz, DMSO-D₆) δ_{H}: 3.20 (1H, broad), 4.45 (2H, broad), 6.27 (1H, broad), 6.39 (1H, broad), 6.73 (2H, d, J=8.6 Hz), 7.36 (2H, d, J=8.6 Hz), 7.57 (1H, broad), 7.80-8.30 (1H, broad), 9.15-9.40 (1H, broad). LCMS t_{R} (min) 1.22. MS (APCI), m/z 299.64 [M+H]⁺.

### 10. 4-[4-[(Furan-2-ylmethyl)-amino]-6-(4-methoxy-phenylamino)-[1,3,5]triazin-2-ylamino]-phenol

¹H-NMR (400 MHz, DMSO-D₆) δ_{H}: 3.70 (3H, s), 4.45 (2H, d, J=7.5 Hz), 6.22 (1H, broad), 6.37 (1H, broad), 6.64 (2H, d, J=8.5 Hz), 6.81 (2H, d, J=8.5 Hz), 7.18 (1H, broad), 7.44 (2H, d, J=8.5 Hz), 7.53 (1H, d, J=1.8 Hz), 7.61 (2H, d, J=8.5 Hz), 8.50-8.80 (2H, broad), 8.89 (1H, s). LCMS t_{R} 2.12 (min). MS (APCI), m/z 405.05 [M+H]⁺. Mₚ 55-57° C.

### 11. 4-[4-[(Furan-2-ylmethyl)-amino]-6-(4-morpholin-4-yl-phenylamino)-[1,3,5]triazin-2-ylamino]-phenol

¹H-NMR (400 MHz, DMSO-D₆) δ_{H}: 3.00 (4H, m), 3.20 (1H, broad), 3.72 (4H, m), 4.44 (2H, d, J=7.5 Hz, broad), 6.22 (1H, broad, Z/E forms), 6.35 (1H, broad, Z/E forms), 6.63 (2H, d, J=8.5 Hz), 6.82 (2H, d, J=8.5 Hz), 7.14 (1H, broad peak, Z/E forms), 7.43 (2H, d, J=8.5 Hz), 7.52 (1H, d, J=1.5 Hz), 7.55 (2H, d, J=8.5 Hz), 8.50-8.80 (2H, broad, Z/E forms). LCMS t_{R} 2.21 (min). MS (APCI), m/z 460.16 [M+H]⁺. Mₚ 162-164° C.

### 12. 4-{4-[(Furan-2-ylmethyl)-amino]-6-[(4-methoxy-phenyl)-methyl-amino]-[1,3,5]triazin-2-ylamino}-phenol

¹H-NMR (400 MHz, DMSO-D₆) δ_{H}: 3.34 (3H, s), 3.76 (3H, s), 4.44 (2H, broad, Z/E forms), 6.22 (1H, broad, Z/E forms), 6.33 (1H, broad, Z/E forms), 6.51 (2H, d, J=8.5 Hz), 6.91 (2H, d, J=8.5 Hz), 7.06 (1H, broad peak), 7.19 (2H, d, J=8.5 Hz), 7.33 (2H, d, J=8.5 Hz), 7.48 (1H, d, J=1.5 Hz), 8.50 (1H, broad, Z/E forms), 8.77 (1H, s). LCMS t_{R} 1.53 (min). MS (APCI), m/z 418.91 [M+H]⁺. Mₚ 63-65° C.

### 13. 4-[4-[(Furan-2-ylmethyl)-amino]-6-(6-methoxy-3,4-dihydro-2H-quinolin-1-yl)-[1,3,5]triazin-2-ylamino]-phenol

¹H-NMR (400 MHz, DMSO-D₆) δ_{H}: 1.85 (2H, t, J=7.5 Hz), 2.73 (2H, t, J=7.5 Hz), 3.72 (3H, s), 3.94 (2H, broad, Z/E forms), 4.45 (2H, broad, Z/E forms), 6.22 (1H, broad, Z/E forms), 6.37 (1H, broad, Z/E forms), 6.64 (2H, d, J=8.5 Hz), 6.70 (2H, broad), 7.41 (2H, d, J=8.5 Hz), 7.45 (1H, broad), 7.54 (1H, s), 7.63 (1H, d, J=8.5 Hz), 8.80-9.10 (2H, broad). LCMS t_{R} 1.62 (min). MS (APCI), m/z 444.84 [M+H]⁺. Mₚ 67-69° C.

### 14. {4-[4-[(Furan-2-ylmethyl)-amino]-6-(4-hydroxy-phenylamino)[1,3,5]triazin-2-ylamino]-phenyl}-morpholin-4-yl-methanone

¹H-NMR (400 MHz, DMSO-D₆) δ_{H}: 3.48 (2H, m), 3.58 (2H, m), 4.49 (2H, d, J=7.5 Hz), 6.22 (1H, broad, Z/E forms), 6.37 (1H, broad, Z/E forms), 6.66 (2H, d, J=8.5 Hz), 7.27 (2H, d, J=8.5 Hz), 7.32 (1H, broad peak, Z/E forms), 7.42 (2H, d, J=8.5 Hz), 7.53 (1H, d, J=1.5 Hz), 7.82 (2H, d, J=8.5 Hz), 8.74 (1H, broad, Z/E forms), 8.91 (1H, s), 9.2 (1H, broad). LCMS t_{R} 1.43 (min). MS (APCI), m/z 487.70 [M+H]⁺. Mₚ 61-63° C.

### 15. 4-[4-[(Furan-2-ylmethyl)-amino]-6-(4-[1,2,4]triazol-4-yl-phenylamino)-[1,3,5]triazin-2-ylamino]-phenol

¹H-NMR (400 MHz, DMSO-D₆) δ_{H}: 4.46-4.54 (2H, m, Z/E forms), 6.28 (1H, broad, Z/E forms), 6.37 (1H, broad, Z/E forms), 6.70 (2H, d, J=8.5 Hz), 7.43 (2H, d, J=8.5 Hz), 7.50-7.58 (3H, m), 7.64 (1H, broad), 7.83 (2H, d, J=8.5 Hz), 9.00-9.08 (3H, m), 9.40 (1H, broad). LCMS t_{R} 1.90 (min). MS (APCI), m/z 442.07 [M+H]⁺. Mₚ 241-243° C.

### 16. 4-{4-(4-Ethoxy-3-trifluoromethyl-phenylamino)-6-[(furan-2-ylmethyl)-amino]-[1,3,5]triazin-2-ylamino}-phenol

¹H-NMR (400 MHz, DMSO-D₆) δ_{H}: 1.30 (3H, t, J=7.5 Hz), 4.12 (2H, q, J=7.5 Hz), 4.47 (2H, d, J=7.5 Hz), 6.21 (1H, broad), 6.36 (1H, broad), 6.64 (2H, d, J=8.5 Hz), 7.12 (1H, d, J=8.5 Hz), 7.27 (1H, broad), 7.43 (2H, d, J=8.5 Hz), 7.52 (1H, d, J=1.8 Hz), 7.80-8.10 (2H, broad), 8.65 (1H, broad), 8.91 (1H, s), 8.85-9.05 (1H, broad). LCMS t_{R} 2.55 (min). MS (APCI), m/z 487.05 [M+H]⁺. Mₚ 46-48° C.

### 17. 2-(4-Hydroxy-phenylamino)-4-[(furan-2-ylmethyl)-amino]-6-morpholin-4-yl-[1,3,5]triazine

A mixture of compound 45 (100 mg, 0.3 mmol) and morpholine (0.2 mL, 2.3 mmol) was stirred at 120° C. for 1 hour (TLC control), cooled to room temperature and diluted with water (50 mL). The formed solid was collected by filtration, washed with water and diethyl ether to give the product (80 mg, 22%).

¹H-NMR (400 MHz, DMSO-D₆) δ_{H}: 3.52-3.60 (4H, m), 3.60-3.70 (4H, m), 4.42 (2H, d, J=7.5 Hz), 6.20 (1H, dd, J=3.6, 1.8 Hz), 6.35 (1H, d, J=3.6 Hz), 6.63 (2H, d, J=8.5 Hz), 7.12 (1H, broad), 7.41 (2H, d, J=8.5 Hz), 7.50 1H, d, J=8.5 Hz), 8.55 (1H, broad), 8.88 (1H, s, broad). LCMS t_{R} (min) 1.87. MS (APCI), m/z 369.04 [M+H]⁺. Mₚ 163-165° C.

### 18. 2-(4-Hydroxy-phenylamino)-4-[(furan-2-ylmethyl)-amino]-6-(4-methyl-piperazin-1-yl)-[1,3,5]triazine

A mixture of compound I-45 (130 mg, 0.4 mmol), N-methylpiperizine (0.2 mL, 2 mmol), triethylamine (0.14 mL, 1 mmol) and ethanol (2 mL) was stirred at refluxing for 1 hour (TLC control), diluted with water (20 mL). The formed solid was collected by filtration, washed with water and diethyl ether to give desired product (75 mg, 20%).

¹H-NMR (400 MHz, DMSO-D₆) δ_{H}: 2.18 (3H, s), 2.21-2.30 (4H, m), 3.64-3.70 (4H, m), 4.44 (2H, d, J=7.5 Hz), 6.20 (1H, dd, J=3.6, 1.8 Hz), 6.38 (1H, d, J=3.8 Hz), 6.63 (2H, d, J=8.5 Hz), 7.07 (1H, broad), 7.41 (2H, d, J=8.5 Hz), 7.52 (1H, d, J=1.8 Hz), 8.52 (1H, broad), 8.86 (1H, s). LCMS t_{R} (min) 1.48. MS (APCI), m/z 382.05 [M+H]⁺. Mₚ 116-118° C.

### 19. 2-(4-Hydroxy-phenylamino)-4-[(furan-2-ylmethyl)-amino]-6-imidazol-1-yl-[1,3,5]triazine

A mixture of compound I-45 (140 mg, 0.44 mmol) and imidazole (250 mg, 3.7 mmol) was fused at 200° C. for 1 hour, cooled to room temperature and treated with water (50 mL). The formed solid was collected by filtration, washed with water, ethanol and diethyl ether to give desired product (115 mg, 33%).

¹H-NMR (400 MHz, DMSO-D₆) δ_{H}: 4.53 (2H, d, J=8.5, broad), 6.18-6.35 (1H, broad, Z/E forms), 6.39 (1H, d, J=3.6 Hz), 6.70 (2H, d, J=8.5 Hz), 7.18 (1H, d, J=1.5 Hz), 7.45 (2H, d, J=8.5, broad), 7.55 (1H, d, J=1.8 Hz), 7.70-7.85 (1H, broad, Z/E forms), 8.00-8.30 (1H, broad, Z/E form), 8.30-8.50 (1H, broad, Z/E form a), 9.07 (1H, s), 9.35-9.60 (1H, broad, Z/E forms). LCMS t_{R} (min) 1.77. MS (APCI), m/z 350.08 [M+H]⁺. Mₚ 244-246° C.

### 20. 4-[4-[(Furan-2-ylmethyl)-amino]-6-(2-methyl-benzothiazol-6-ylamino)-[1,3,5]triazin-2-ylamino]-phenol

¹H-NMR (400 MHz, DMSO-D₆) δ_{H}: 2.72 (3H, s), 4.49 (2H, d, J=7.5 Hz), 6.25 (1H, broad, Z/E forms), 6.37 (1H, broad, Z/E forms), 6.68 (2H, d, J=8.5 Hz), 7.35 (1H, broad, Z/E forms), 7.44 (2H, d, J=8.5 Hz), 7.54 (1H, s), 7.60 (1H, broad, Z/E forms), 7.72 (1H, d, J=8.5 Hz), 8.66 (1H, broad, Z/E forms), 8.77 (1H, broad, Z/E forms), 8.97 (1H, s), 9.15 (1H, broad, Z/E forms). LCMS t_{R} 1.50 (min). MS (APCI), m/z 445.74 [M+H]⁺. Mₚ 68-70° C.

### 21. 5-[4-[(Furan-2-ylmethyl)-amino]-6-(4-hydroxy-phenylamino)[1,3,5]triazin-2-ylamino]-1,3-dihydro-benzoimidazol-2-one

¹H-NMR (400 MHz, DMSO-D₆) δ_{H}: 4.48 (2H, d, J=7.5 Hz), 6.23 (1H, dd, J=3.6 1.8 Hz), 6.37 (1H, d, J=3.6 Hz), 6.64 (2H, d, J=8.5 Hz), 6.77 (1H, d, J=8.5 Hz), 7.10 (1H, broad), 7.25 (1H, broad), 7.29 (1H, d, J=8.5 Hz), 7.47 (2H, d, =8.5 Hz), 7.52 (1H, d, J=1.8 Hz), 8.56 (1H, broad), 8.68 (1H, broad), 8.88 (1H, s), 10.25 (1H, s), 10.32 (1H, s). LCMS t_{R} 1.33 (min). MS (APCI), m/z 430.98 [M+H]⁺.

### 22. 4-[4-[(Furan-2-ylmethyl)-amino]-6-(1H-indol-5-ylamino)-[1,3,5]-triazin-2-ylamino]-phenol

¹H-NMR (400 MHz, DMSO-D₆) δ_{H}: 4.48 (2H, q, J=7.5 Hz), 6.22 (1H, broad), 6.30 (1H, broad), 6.36 (1H, broad), 6.63 (2H, d, J=8.5 Hz), 7.08 (1H, broad), 7.22 (1H, s), 7.20-7.30 (2H, m), 7.46 (2H, d, J=8.5 Hz), 7.53 (1H, s), 7.92 (1H, s), 8.60 (2H, broad), 8.86 (1H, s), 10.78 (1H, s). LCMS t_{R} 1.45 (min). MS (APCI), m/z 413.76 [M+H]⁺. Mₚ 54-56° C.

### 23. 4-[4-[(Furan-2-ylmethyl)-amino]-6-(1H-indol-6-ylamino)-[1,3,5]triazin-2-ylamino]-phenol

^{23. 1}H-NMR (400 MHz, DMSO-D₆) δ_{H}: 4.50 (2H, broad), 6.23 (1H, broad), 6.34 (1H, broad), 6.37 (1H, broad), 6.63 (2H, d, J=8.5 Hz), 7.14 (1H, broad), 7.20 (1H, s), 7.32 (1H, d, J=8.5 Hz), 7.38 (1H, d, J=8.5 Hz), 7.50 (2H, d, J=8.5 Hz), 7.54 (1H, s), 7.72 (1H, s), 8.60 (1H, broad), 8.77 (1H, broad), 8.86 (1H, s), 10.77 (1H, s). LCMS t_{R} 1.50 (min). MS (APCI), m/z 413.77 [M+H]⁺. Mₚ 53-55° C.

### 24. 4-[4-[(Furan-2-ylmethyl)-amino]-6-(1H-indazol-5-ylamino)-[1,3,5]triazin-2-ylamino]-phenol

¹H-NMR (400 MHz, DMSO-D₆) δ_{H}: 4.49 (2H, d, J=7.5 Hz), 6.23 (1H, broad, Z/E forms), 6.37 (1H, broad, Z/E forms), 6.66 (2H, d, J=8.5 Hz), 7.22 (1H, broad), 7.39 (1H, d, J=8.5 Hz), 7.45 (2H, d, J=8.5 Hz), 7.50-7.57 (2H, m), 7.90 (1H, s), 8.19 (1H, s), 8.67 (1H, broad), 8.89 (1H, broad), 8.94 (1H, s), 12.80 (1H, s). LCMS t_{R} 1.35 (min). MS (APCI), m/z 414.76 [M+H]⁺. Mₚ 78-80° C.

### 5-[4-[(Furan-2-ylmethyl)-amino]-6-(4-hydroxy-phenylamino)-[1,3,5]triazin-2-ylamino]-isoindole-1,3-dione

¹H-NMR (400 MHz, DMSO-D₆) δ_{H}: 4.45 (2H, broad, Z/E forms), 6.25 (1H, broad), 6.37 (1H, broad), 6.67 (2H, d, J=8.5 Hz), 7.35-7.50 (1H, broad, Z/E forms), 7.45 (2H, d, J=8.5 Hz), 7.53 (1H, s), 7.62 (1H, d, J=8.5 Hz), 8.18 (2H, broad), 8.70-9.00 (1H, broad, Z/E forms), 8.95 (1H, s), 9.57 (1H, broad), 1.95 (1H, broad). LCMS t_{R} 1.48 (min). MS (APCI), m/z 443.83 [M+H]⁺. Mₚ 162-164° C.

### 26. 4-{4-(2,3-Dihydro-benzo[1,4]dioxin-6-ylamino)-6-[(furan-2-ylmethyl)-amino]-[1,3,5]triazin-2-ylamino}-phenol

¹H-NMR (400 MHz, DMSO-D₆) δ_{H}: 4.21 (4H, m), 4.48 (2H, broad, Z/E forms), 6.22 (1H, broad), 6.38 (1H, broad), 6.66 (2H, d, J=8.5 Hz), 6.70 (1H, d, J=8.5 Hz), 7.14 (1H, d, J=8.5 Hz), 7.21 (1H, broad), 7.37 (1H, s), 7.48 (2H, d, J=8.5 Hz), 7.53 (1H, s), 8.67 (2H, broad, Z/E forms), 8.89 (1H, s). LCMS t_{R} 1.49 (min). MS (APCI), m/z 433.12 [M+H]⁺. Mₚ 44-46° C.

### 27. 7-[4-[(Furan-2-ylmethyl)-amino]-6-(4-hydroxy-phenylamino)-[1,3,5]triazin-2-ylamino]-4H-benzo[1,4]oxazin-3-one

¹H-NMR (400 MHz, DMSO-D₆) δ_{H}: 4.45 (2H, d, J=7.5 Hz), 4.50 (2H, s), 6.24 (1H, broad, Z/E forms), 6.36 (1H, broad, Z/E forms), 6.65 (2H, d, J=8.5 Hz), 6.74 (1H, d, J=8.5 Hz), 7.27 (2H, broad, Z/E forms), 7.43 (2H, d, J=8.5 Hz), 7.52 (1H, d, J=8.5 Hz), 7.53 (1H, s), 8.69 (1H, broad, Z/E forms), 8.85 (1H, broad, Z/E forms), 8.91 (1H, s), 10.40 (1H, s). LCMS t_{R} 1.31 (min). MS (APCI), m/z 445.71 [M+H]⁺. Mₚ 54-56° C.

### 28. 6-[4-[(Furan-2-ylmethyl)-amino]-6-(4-hydroxy-phenylamino)-[1,3,5]triazin-2-ylamino]-3-methyl-3H-benzooxazol-2-one

¹H-NMR (400 MHz, DMSO-D₆) δ_{H}: 3.30 (3H, s), 4.49 (2H, broad, Z/E forms), 6.22 (1H, broad), 6.37 (1H, broad), 6.67 (2H, d, J=8.5 Hz), 7.08 (1H, d, J=8.5 Hz), 7.30 (1H, broad), 7.42 (1H, d, J=8.5 Hz), 7.46 (2H, d, J=8.5 Hz), 7.55 (1H, s), 8.05 (1H, broad), 8.73 (1H, broad), 8.94 (1H, s), 9.00 (1H, broad). LCMS t_{R} 1.45 (min). MS (APCI), m/z 446.05 [M+H]⁺. Mₚ 126-129° C.

### 29. 4-[4-[(Furan-2-ylmethyl)-amino]-6-(3-phenoxy-phenylamino)-[1,3,5]triazin-2-ylamino]-phenol

PBu^{t}₃ (0.2 mg, 7 mol %) was added to a solution of Pd₂(dba)₃ (37 mg, 5 mol %) in toluene (1 mL). The solution was stirred at room temperature for 15 minutes. Then it was transferred to a solution of I-45 (250 mg, 0.78 mmol), aniline (290 mg, 1.57 mmol) and NaOBu^{t} (160 mg, 1.7 mmol) in toluene (3 mL). The obtained mixture was stirred at refluxing for 4 hours, diluted with water (20 mL), extracted with ethyl acetate (3×10 mL). The combined organic fractions were dried over sodium sulfate and concentrated. Purification by column chromatography (silica gel, ethyl acetate/hexane) gave the product (37 mg, 8%).

¹H-NMR (400 MHz, DMSO-D₆) δ_{H}: 4.40 (2H, broad), 6.21 (1H, broad), 6.34 (1H, broad), 6.53 (1H, broad, Z/E forms), 6.65 (2H, d, J=8.5 Hz), 6.99 (2H, J=8.5 Hz), 7.09 (1H, t, J=8.5 Hz), 7.20 (1H, t, J=8.5 Hz), 7.25 (1H, broad), 7.35 (2H, t, J=8.5 Hz), 7.45 (2H, d, J=8.5 Hz), 7.51 (1H, s), 7.55 (2H, broad), 8.55-8.75 (1H, broad, Z/E forms), 8.90 (1H, s), 9.00 (1H, broad). LCMS t_{R} 1.81 (min). MS (APCI), m/z 467.09 [M+H]⁺. Mₚ 80-83° C.

### 30. 4-[4-[(Furan-2-ylmethyl)-amino]-6-(4-hydroxy-phenylamino)[1,3,5]triazin-2-ylamino]-benzoic acid ethyl ester

¹H-NMR (400 MHz, DMSO-D₆) δ_{H}: 1.30 (3H, t, J=7.5 Hz), 4.28 (2H, d, J=7.5 Hz), 4.51 (2H, broad), 6.25 (1H, broad), 6.38 (1H, broad), 6.70 (2H, d, J=8.5 Hz), 7.40 (1H, broad), 7.44 (2H, d, J=8.5 Hz), 7.54 (1H, s), 7.81 (2H, d, J=8.5 Hz), 7.93 (2H, d, J=8.5 Hz), 8.80 (1H, broad), 8.96 (1H, s), 9.32 (1H, broad). LCMS t_{R} 1.68 (min). MS (APCI), m/z 446.83 [M+H]⁺.

### 31. 4-[4-[(Furan-2-ylmethyl)-amino]-6-(4-phenoxy-phenylamino)-[1,3,5]triazin-2-ylamino]-phenol

¹H-NMR (400 MHz, DMSO-D₆) δ_{H}: 4.48 (2H, d, J=7.5 Hz), 6.23 (1H, broad), 6.36 (1H, broad), 6.65 (2H, d, J=8.5 Hz), 6.91 (2H, J=8.5 Hz), 6.95 (2H, d, J=8.5 Hz), 7.06 (1H, t, J=8.5 Hz), 7.23 (1H, broad), 7.35 (2H, t, J=8.5 Hz), 7.45 (2H, d, J=8.5 Hz), 7.52 (1H, s), 7.77 (2H, d, J=8.5 Hz), 8.65 (1H, broad), 8.89 (1H, s), 8.90 (1H, broad). LCMS t_{R} 1.73 (min). MS (APCI), m/z 446.83 [M+H]⁺. Mₚ 103-105° C.

### 32. 4-[4-[(Furan-2-ylmethyl)-amino]-6-(4-hydroxy-phenylamino)-[1,3,5]triazin-2-ylamino]-benzonitrile

¹H-NMR (400 MHz, DMSO-D₆) δ_{H}: 4.50 (2H, d, J=7.5 Hz), 6.25 (1H, broad), 6.38 (1H, broad), 6.69 (2H, d, J=8.5 Hz), 7.45 (2H, d, J=8.5 Hz), 7.49 (1H, broad), 7.55 (1H, s), 7.62 (2H, d, J=8.5 Hz), 7.98 (2H, broad), 8.75-9.00 (1H, broad, Z/E forms), 8.96 (1H, s), 9.44 (1H, broad). LCMS t_{R} 1.62 (min). MS (APCI), m/z 399.79 [M+H]⁺.
33. N-Furan-2-ylmethyl-N′-(2-methyl-benzothiazol-6-yl)-N″-(1-methyl-piperidin-4-yl)-[1,3,5]triazine-2,4,6-triamine

A mixture of compound I-6 (1.000 g, 2.68 mmol), 1-Methyl-piperidin-4-ylamine (306 mg, 2.68 mmol), K₂CO₃ (1.100 g, 8.00 mmol) and DMSO (1 mL) was stirred for 3 hours at 150° C., cooled to room temperature and diluted with water. The formed solid was collected by filtration. Purification by column chromatography (silica gel, methanol/ethyl acetate) and preparative TLC gave the product (32 mg, 3%).

¹H-NMR (400 MHz, DMSO-D₆) δ_{H}: 1.62 (2H, m), 1.88 (2H, m), 2.20-2.40 (5H, m), 2.72 (3H, s), 2.94 (2H, broad), 3.81 (1H, broad), 4.48 (2H, d, J=7.5 Hz), 6.22 (1H, dd, J=3.6 1.8 Hz), 6.38 (1H, d, J=3.6 Hz), 6.78 (1H, broad), 7.12 (1H, broad, Z/E forms), 7.52 (1H, d, J=1.8 Hz), 7.62 (1H, d, J=5.0 Hz), 7.71 (1H, d, J=8.5 Hz), 8.66 (1H, broad), 8.80-9.20 (1H, broad, Z/E forms). LCMS t_{R} 1.37 (min). MS (APCI), m/z 451.06 [M+H]⁺. Mₚ 62-65° C.

### 34. 4-[4-[(Furan-2-ylmethyl)-amino]-6-(4-hydroxy-phenoxy)-[1,3,5]triazin-2-ylamino]-phenol

A mixture of 45 (250 mg, 0.78 mmol), p-hydroxyphenol (257 mg, 2.34 mmol), potassium carbonate (414 mg, 3 mmol) and DMSO (200 μl) was stirred for 4 hours at 180° C., diluted with water (20 mL) and extracted with ethyl acetate (2×10 mL). The combined organic phases were concentrated at reduced pressure. Purification by column chromatography (silica gel, ethyl acetate hexane) gave the product (40 mg, 13%). ¹H-NMR (400 MHz, DMSO-D₆) δ_{H}: 4.30-4.50 (2H, broad, Z/E forms), 6.05-6.30 (1H, broad, Z/E forms), 6.35 (1H, t, J=3.6 Hz), 6.60 (2H, broad), 6.74 (2H, d, J=8.5 Hz), 6.94 (2H, d, J=8.5 Hz), 7.35 (2H, broad), 7.52 (1H, d, J=1.8 Hz), 7.73 (1H, broad), 8.92 (1H, s), 8.88-9.20 (1H, broad, Z/E forms), 9.20 (1H, s). LCMS t_{R} 1.44 (min). MS (APCI), m/z 391.98 [M+H]⁺. Mₚ 80-83° C.

### 35. 4-{4-Ethylamino-6-[(furan-2-ylmethyl)-amino]-[1,3,5]triazin-2-ylamino}-phenol

A mixture of I-45 (250 mg, 0.78 mmol), NH₂Et*HCl (300 mg, 3.7 mmol), DIPEA (5 mL) was stirred for 2 hours at 100° C., cooled down to room temperature, diluted with water (20 mL). The formed solid was collected by filtration and recrystallized twice from ethyl acetate/hexane to give the product (110 mg, 43%). ¹H-NMR (400 MHz, DMSO-D₆) δ_{H}: 1.09 (3H, t, J=7.5 Hz), 3.38 (2H, q, J=7.5 Hz), 4.42 (2H, d, J=7.5 Hz), 6.20 (1H, broad), 6.36 (1H, broad), 6.55 (1H, broad), 6.62 (2H, d, J=8.5 Hz), 6.90 (1H, broad), 7.46 (2H, d, J=8.5 Hz), 7.50 (1H, s), 8.43 (1H, broad), 8.81 (1H, s). LCMS t_{R} 1.44 (min). MS (APCI), m/z 327.02 [M+H]⁺. Mₚ 76-78° C.

### 36. 4-{4-[(Furan-2-ylmethyl)-amino]-6-[(thiophen-2-ylmethyl)-amino]-[1,3,5]triazin-2-ylamino}-phenol

¹H-NMR (400 MHz, DMSO-D₆) δ_{H}: 4.42 (2H, broad), 4.58 (2H, d, J=7.5 Hz), 6.19 (1H, broad), 6.32 (1H, broad), 6.60 (2H, d, J=8.5 Hz), 6.91 (1H, broad), 6.95 (1H, broad), 7.05 (1H, broad), 7.15 (1H, broad), 7.28 (1H, d, J=8.5 Hz), 7.43 (2H, d, J=8.5 Hz), 7.49 (1H, s), 8.50 (1H, broad), 8.81 (1H, s). LCMS t_{R} 1.51 (min). MS (APCI), m/z 395.03 [M+H]⁺. Mₚ 77-79° C.

### 37. 4-{4-(4-Bromo-phenylamino)-6-[(furan-2-ylmethyl)-amino]-[1,3,5]triazin-2-ylamino}-phenol

A mixture of compound 45 (317 mg, 1 mmol), 4-bromoaniline (172 mg, 1 mmol), anhydrous K₂CO₃ (415 mg, 3 mmol) and DMSO (200 μL) was heated at 150° C. for 2 hours. After cooling to room temperature the resulting mixture was washed with hot water (50 mL) and extracted with hot ethyl acetate (2×30 mL). The combined organic phases were dried over Na₂SO₄ and concentrated. Purification by column chromatography on silica gel (dichloromethane) gave the product (140 mg, 31%). ¹H-NMR (400 MHz, DMSO-D₆) δ_{H}: 4.49 (2H, d, J=7.5 Hz), 6.24 (1H, t, J=3.6/1.8 Hz), 6.39 (1H, d, J=3.6 Hz), 6.69 (2H, d, J=8.5 Hz), 7.36 (1H, broad), 7.36 (2H, d, J=8.5 Hz), 7.45 (2H, d, J=8.5 Hz), 7.54 (1H, d, J=1.8 Hz), 7.75 (2H, broad), 8.72 (1H, broad), 8.94 (1H, s), 9.08 (1H, broad). LCMS t_{R} (min): 1.71. MS (APCI), m/z 452.87, 454.89 [M+H]⁺. HPLC t_{R} (min): 11.92. Mₚ 37-39° C.

### 38. N-Ethyl-N′-(4-methoxy-phenyl)-N″-(2-pyrrolidin-1-yl-ethyl)-[1,3,5]triazine-2,4,6-triamine

A mixture of compound I-25 (280 mg, 1.0 mmol), 2-pyrrolidin-1-yl-ethylamine (0.127 mL, 1.0 mmol), DIPEA (0.174 mL, 1.0 mmol) and acetonitrile (17 mL) was stirred at 70° C. for 12 hours, cooled down to room temperature, diluted with water, extracted with dichloromethane. The combined organic phases were dried over sodium sulfate, concentrated at reduced pressure and dried to give the product (138, mg, 38%). ¹H-NMR (400 MHz, DMSO-D₆) δ_{H}: 1.18 (3H, t, J=7.5 Hz), 1.68 (4H, m), 2.51 (2H, t, J=7.5 Hz), 3.22 (6H, m), 3.35 (2H, broad), 3.71 (3H, s), 6.20-6.70 (2H, broad, Z/E forms), 6.76 (2H, d, J=8.5 Hz), 7.61 (2H, d, J=8.5 Hz, broad), 8.55 (1H, broad). LCMS t_{R} (min): 1.25. MS (APCI), m/z 358.15 [WH]. HPLC t_{R} (min): 7.64. Mₚ 29-31° C.

### 39. N-Ethyl-N′-furan-2-ylmethyl-N″-(2-methyl-benzothiazol-6-yl)-[1,3,5]triazine-2,4,6-triamine

A mixture of compound I-6 (372 mg, 1.0 mmol), ethylamine hydrochloride (82 mg, 1.0 mmol), K₂CO₃ (417 mg, 3.0 mmol) and dioxane (5 mL) was stirred at 80° C. for 8 hours and cooled to room temperature. Then the obtained mixture was transferred to column (silica gel, methanol/dichloromethane). Additional purification by preparative TLC (ethyl acetate) gave the product (35 mg, 9%). ¹H-NMR (400 MHz, DMSO-D₆) δ_{H}: 1.10 (3H, t, J=7.5 Hz), 2.73 (3H, s), 3.28 (2H, q, J=7.5 Hz), 4.48 (2H, d, J=7.5 Hz), 6.22 (1H, broad), 6.36 (1H, broad), 6.77 (1H, broad), 7.18 (1H, broad), 7.53 (1H, s), 7.67 (1H, d, J=8.5 Hz, broad), 7.72 (1H, d, J=8.5 Hz), 8.60-8.75 (1H, broad, Z/E forms), 8.80-9.15 (1H, broad, Z/E forms). LCMS t_{R} (min): 1.52. MS (APCI), m/z [M+H]⁺ 381.99. HPLC t_{R} (min): 10.40. Mₚ 97-99° C.

### 40. N-(2-Dimethylamino-ethyl)-N′-furan-2-ylmethyl-N″-(2-methyl-benzothiazol-6-yl)-[1,3,5]triazine-2,4,6-triamine

¹H-NMR (400 MHz, DMSO-D₆) δ_{H}: 2.19 (6H, s), 2.41 (2H, t, J=7.5 Hz), 2.72 (3H, s), 3.38 (2H, broad), 4.49 (2H, d, J=7.5 Hz), 6.23 (1H, broad), 6.38 (1H, broad), 6.60 (1H, broad), 7.18 (1H, broad), 7.52 (1H, s), 7.64 (1H, d, J=8.5 Hz), 7.71 (1H, d, J=8.5 Hz), 8.67 (1H, broad), 9.04 (1H, broad). LCMS t_{R} (min): 1.44. MS (APCI), m/z 425.11 [M+H]⁺. HPLC t_{R} (min): 8.24. Mₚ 92-94° C.

### 41. N-Furan-2-ylmethyl-N′-(4-methoxy-phenyl)-[1,3,5]triazine-2,4-diamine

A mixture of compound I-31 (470 mg, 1.9 mmol), furfuryl amine (194 mg, 2 mmol), K₂CO₃ (278 mg, 4 mmol) and DMSO (1 mL) was stirred at 90° C. for 10 hours, cooled to room temperature and diluted with water. The formed solid was collected by filtration, recrystallized from CHCl₃/MeOH and purified via preparative TLC (ethyl acetate/hexane) to give the product (300 mg, 50%). ¹H-NMR (400 MHz, DMSO-D₆) δ_{H}: 3.74 (3H, s), 4.49 (2H, broad, Z/E forms), 6.24 (2H, broad), 6.38 (1H, broad), 6.85 (2H, d, J=8.5 Hz), 7.54 (1H, d, J=1.5 Hz), 7.59 (2H, broad, Z/E forms), 7.71-7.89 (1H, broad, Z/E forms), 8.08-8.23 (1H, broad, Z/E forms), 9.19-9.42 (1H, broad, Z/E forms). LCMS t_{R} (min): 1.54. MS (APCI), m/z 298.08 [M+H]⁺. HPLC t_{R} (min): 9.65. Mₚ 184-186° C.

### 42. N-Furan-2-ylmethyl-N′-(3-trifluoromethyl-phenyl)-[1,3,5]triazine-2,4-diamine

A solution of m-CF₃-aniline (322 mg, 2 mmol) and DIPEA (258 mg, 2 mmol) in DMSO (0.5 mL) was added dropwise to a solution of dichlorotriazine (300 mg, 2 mmol) in DMSO (0.5 mL) at 10° C. The reaction mixture was stirred at room temperature for 1 hour. Then furfuryl amine (194 mg, 2 mmol) and K₂CO₃ (278 mg, 2 mmol) were added. The obtained mixture was stirred for 1 hour at 100° C., cooled down to room temperature and diluted with water. The formed solid was collected by filtration, purified by column chromatography (silica gel, ethyl acetate), preparative TLC (ethyl acetate) and recrystallized from ethyl acetate to give the product (70 mg, 10%). ¹H-NMR (400 MHz, DMSO-D₆) δ_{H}: 4.52 (2H, d, J=7.5 Hz), 6.24 (1H, broad), 6.37 (1H, broad), 7.30 (1H, d, J=8.5 Hz), 7.50 (1H, t, J=8.5 Hz), 7.53 (1H, s), 7.85-8.00 (1H, broad, Z/E forms), 8.11 (1H, broad), 8.11-8.25 (1H, broad, Z/E forms), 8.31 (1H, broad), 9.70-9.90 (1H, broad, Z/E forms). LCMS t_{R} (min): 1.79. MS (APCI), m/z 336.04 [M+H]⁺. HPLC t_{R} (min): 12.53. Mₚ 207-209° C.

### 43. N-Furan-2-ylmethyl-N′,N′-dimethyl-N″-(3-trifluoromethyl-phenyl)-[1,3,5]triazine-2,4,6-triamine

A mixture of compound I-28 (500 mg, 1.4 mmol), dimethylamine hydrochloride (325 mg, 4 mmol), triethylamine (550 mg, 5.42 mmol) and acetonitrile (5 mL) was stirred at 50° C. for 4 hours, cooled down to room temperature, concentrated at reduced pressure, diluted with water and ectracted with chloroform. The combined organic phases were concentrated at reduced pressure. Purification by column chromatography (silica gel, ethyl acetate/hexane) and preparative TLC gave the product (328 mg, 46%).

¹H-NMR (400 MHz, DMSO-D₆) δ_{H}: 3.09 (6H, s), 4.49 (2H, d, J=7.5 Hz), 6.21 (1H, broad), 6.37 (1H, broad), 7.21 (1H, d, J=8.5 Hz), 7.26 (1H, broad), 7.44 (1H, t, J=8.5 Hz), 7.52 (1H, s), 7.89 (1H, broad), 8.41 (1H, broad, Z/E forms), 9.26 (1H, broad). LCMS t_{R} (min): 1.84. MS (APCI), m/z 379.10 [M+H]⁺. HPLC t_{R} (min): 12.92. Mₚ 81-83° C.

### 44. N-(4-Chloro-phenyl)-N′-furan-2-ylmethyl-N″-(4-nitro-phenyl)-[1,3,5]triazine-2,4,6-triamine

¹H-NMR (400 MHz, DMSO-D₆) δ_{H}: 4.56 (2H, d, J=7.5 Hz), 6.30 (1H, dd, J=3.6, 1.8 Hz), 6.40 (1H, broad), 7.32 (2H, d, J=8.5 Hz), 7.57 (1H, s), 7.70-7.90 (3H, m, broad), 8.80-8.20 (4H, m), 9.20-9.40 (1′-1, broad, Z/E forms), 9.75-9.90 (1H, broad, Z/E forms). LCMS t_{R} (min) 2.14. MS (APCI), m/z 437.59 [M+H]⁺.

### 45. 2-(4-Hydroxy-phenylamino)-4-Chloro-6-[(furan-2-ylmethyl)-amino]-[1,3,5]triazine

To a cooled to −20° C. solution of cyanuric chloride (18.44 g, 100 mmol) in anhydrous THF (200 mL) a solution of N,N-diisopropylethylamine (13.57 g, 105 mmol) and furfurylamine (9.71 g, 100 mmol) in anhydrous THF (150 mL) was added dropwise at −20° C. for a period of 30 minutes. The resulting mixture was stirred at −20° C. for 1 hour (TLC control). Then the reaction mixture was allowed to warm up to 0° C. A solution of N,N-diisopropylethylamine (13.57 g, 105 mmol) and p-aminophenol (10.91 g, 100 mmol) in anhydrous THF (200 mL) was added dropwise at 0° C. to the reaction mixture. The resulting mixture was stirred at 0° C. for 2 hours then at room temperature for 10 hours. The solvent was removed at reduced pressure. The crude product was purified by column chromatography on silica gel (methanol/ethyl acetate/hexane) giving I-45 (13.66 g, 43%). ¹H-NMR (400 MHz, DMSO-D₆) δ_{H}: 4.46 (2H, d, J=7.5 Hz); 6.24 (1H, d, J=7.5 Hz, broad), 6.38 (1H, broad), 6.70 (2H, d, J=8.6 Hz), 7.40 (2H, broad), 7.54 (1H, d, J=1.8 Hz), 8.25-8.40 (1H, broad, Z/E forms), 9.20 (1H, broad), 9.50-9.70 (1H, broad, Z/E forms). LCMS t_{R} (min) 1.56. MS (APCI), m/z 317.52, 319.52 [M+H]⁺. Mₚ 188-190° C.

### 46. 6-(2-Dimethylamino-ethoxy)-N-thiophen-2-ylmethyl-N′-(3-trifluoromethyl-phenyl)-[1,3,5]triazine-2,4-diamine

To a solution of N,N-dimethylaminoethanol (89 mg, 1 mmol) in THF (1.5 mL) NaH (60% in oil, 40 mg, 1 mmol) was added at 0° C. The mixture was stirred at 0° C. for 30 min. Then a solution of compound N-thiophen-2-ylmethyl-N′-(3-trifluoromethyl-phenyl)-6-chloro-[1,3,5]triazine-2,4-diamine (385 mg, 1 mmol) in THF (1.5 mL) was added to the resulting mixture at 0° C. Stirring was continued at 0° C. for 30 min. Then the reaction mixture was warmed up to room temperature, stirred at room temperature for 4 hours, concentrated at reduced pressure, diluted with water, extracted with chloroform. The combined organic phases were concentrated at reduced pressure. Purification by preparative TLC gave the compound (82 mg, 19%). ¹H-NMR (400 MHz, DMSO-D₆) δ_{H}: 2.16 (6H, s), 3.93 (2H, broad), 4.32 (2H, broad), 4.65 (2H, broad), 6.92 (1H, broad), 6.98 (1H, broad), 7.26 (1H, d, J=8.5 Hz), 7.33 (1H, broad), 7.46 (1H, t, J=8.5 Hz), 7.85-8.10 (1H, broad, Z/E forms), 8.00 (1H, broad), 8.10-8.30 (1H, broad, Z/E forms), 9.55-9.80 (1H, broad, Z/E forms). LCMS t_{R} (min): 1.62. MS (APCI), m/z 438.82 [M+H]⁺. HPLC t_{R} (min): 11.41. Mₚ 51-53° C.

### 47. N-Furan-2-ylmethyl-N′,N′-dimethyl-N″-(3-trifluoromethyl-phenyl)-[1,3,5]triazine-2,4,6-triamine

A mixture of compound I-8 (500 mg, 1.4 mmol), dimethylamine hydrochloride (325 mg, 4 mmol), triethylamine (550 mg, 5.42 mmol) and acetonitrile (5 mL) was stirred at 50° C. for 4 hours, cooled down to room temperature, concentrated at reduced pressure, diluted with water and extracted with chloroform. The combined organic phases were concentrated at reduced pressure. Purification by column chromatography (silica gel, ethyl acetate/hexane) and preparative TLC gave the compound (328 mg, 46%). ¹H-NMR (400 MHz, DMSO-D₆) δ_{H}: 3.09 (6H, s), 4.49 (2H, d, J=7.5 Hz), 6.21 (1H, broad), 6.37 (1H, broad), 7.21 (1H, d, J=8.5 Hz), 7.26 (1H, broad), 7.44 (1H, t, J=8.5 Hz), 7.52 (1H, s), 7.89 (1H, broad), 8.41 (1H, broad, Z/E forms), 9.26 (1H, broad). LCMS t_{R} (min): 1.84. MS (APCI), m/z 379.10 [M+H]⁺. HPLC t_{R} (min): 12.92. Mₚ 81-83° C.

### Preparation of O,N,N-triazines in Table 6

The “R2-variation” compounds containing O-link at 2-position were synthesized through three step reaction sequence as shown in Scheme 4:

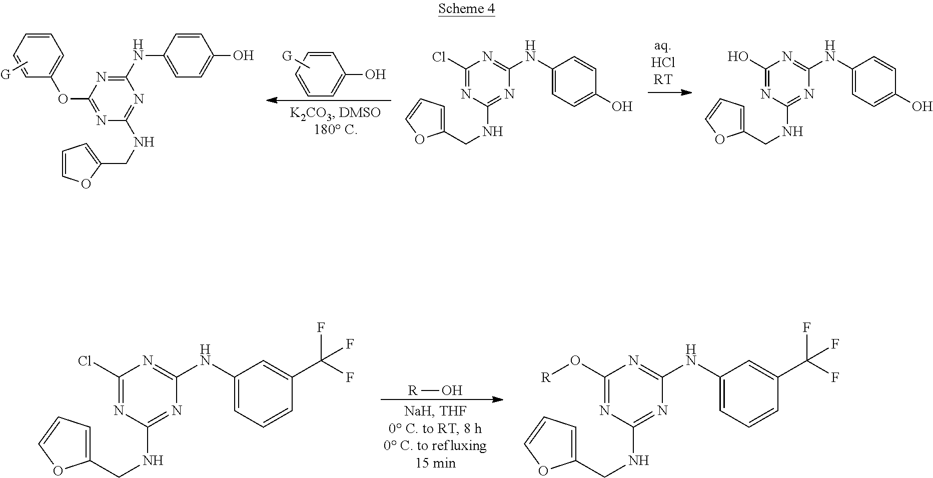

### Generic Procedure for Synthesis of 0,N,N-triazines

NaH (68 mg, 60% in oil, 1.7 mmol) was added to a solution of alcohol (1.7 mmol) in THF (2 mL) at 0° C. The mixture was stirred for 20 minutes at 0° C. Then a solution of mono-chloro-triazine (300 mg, 0.81 mmol) in THF (2 mL) was added to the obtained suspension at 0° C. The final reaction mixture was stirred at room temperature for 8 hours or at refluxing for 15 minutes, diluted with water, extracted with dichloromethane. The combined organic phases were concentrated at reduced pressure and purified by column chromatography to give final compounds.

TABLE 6

| En | Structure | Name0 | MW | Formula |
|---|---|---|---|---|
| 1 | 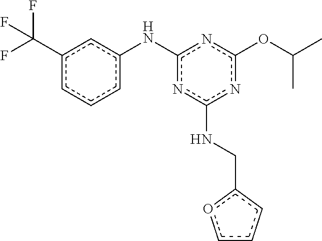 | N-(furan-2-ylmethyl)-6- (propan-2-yloxy)-N′-[3- (trifluoromethyl)phenyl]-1,3,5- triazine-2,4-diamine | 393.4 | C18H18F3N5O2 |
| 2 | 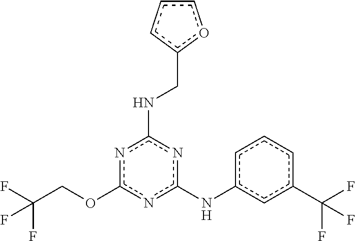 | N-(furan-2-ylmethyl)-6-(2,2,2- trifluoroethoxy)-N′-[3- (trifluoromethyl)phenyl]-1,3,5- triazine-2,4-diamine | 433.3 | C17H13F6N5O2 |
| 3 | 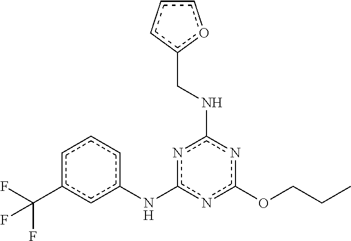 | N-(furan-2-ylmethyl)-6- propoxy-N′-[3- (trifluoromethyl)phenyl]-1,3,5- triazine-2,4-diamine | 393.4 | C18H18F3N5O2 |
| 4 | 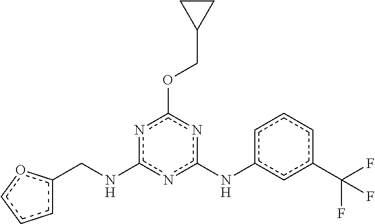 | 6-(cyclopropylmethoxy)-N- (furan-2-ylmethyl)-N′-[3- (trifluoromethyl)phenyl]-1,3,5- triazine-2,4-diamine | 405.4 | C19H18F3N5O2 |
| 5 | 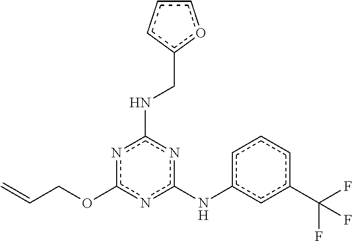 | N-(furan-2-ylmethyl)-6-(prop- 2-en-1-yloxy)-N′-[3- (trifluoromethyl)phenyl]-1,3,5- triazine-2,4-diamine | 391.3 | C18H16F3N5O2 |
| 6 | 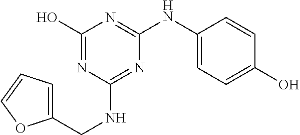 | 4-[(Furan-2-ylmethyl)-amino]- 6-(4-hydroxy-phenylamino)- [1,3,5]triazin-2-ol | 299.1 | C14H13N5O3 |
| 7 | 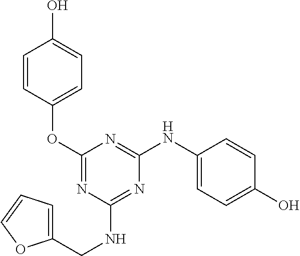 | 4-[4-[(Furan-2-ylmethyl)- amino]-6-(4-hydroxy- phenoxy)-[1,3,5]triazin-2- ylamino]-phenol | 391.1 | C20H17N5O4 |
| 8 | 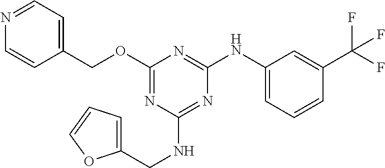 | N-Furan-2-ylmethyl-6-(pyridin- 4-ylmethoxy)-N′-(3- trifluoromethyl-phenyl)- [1,3,5]triazine-2,4-diamine | 442.4 | C21H17F3N6O2 |
| 9 | 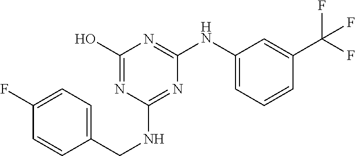 | 4-(4-Fluoro-benzylamino)-6- (3-trifluoromethyl- phenylamino)-[1,3,5]triazin-2- ol | 379.1 | C17H13F4N5O |

### Procedures and Analytical Data for Compounds in Table 6.

### 1. N-Furan-2-ylmethyl-6-isopropoxy-N′-(3-trifluoromethyl-phenyl)-[1,3,5]-triazine-2,4-diamine

¹H-NMR (400 MHz, DMSO-D₆) δ_{H}: 1.30 (6H, d, J=7.5 Hz), 4.50 (2H, broad), 5.20 (1H, m), 6.24 (1H, broad), 6.39 (1H, broad), 7.29 (1H, d, J=8.5 Hz), 7.49 (1H, t, J=8.5 Hz), 7.53 (1H, s), 7.87 (1H, broad), 7.99-8.06 (1H, broad, Z/E forms), 8.13-8.38 (1H, broad, Z/E forms), 9.55-9.74 (1H, broad, Z/E forms). LCMS t_{R} (min): 2.06. MS (APCI), m/z 393.96 [M+H]⁺. HPLC t_{R} (min): 15.45. Mₚ 124-126° C.

### 2. N-Furan-2-ylmethyl-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 4.52 (2H, s), 4.99 (2H, broad), 6.22-6.31 (1H, broad, Z/E forms), 6.40 (1H, broad), 7.33 (1H, d, J=8.5 Hz), 7.50 (1H, broad), 7.53 (1H, s), 7.83-8.09 (1H, broad, Z/E forms), 8.09-8.25 (1H, broad, Z/E forms), 8.15-8.35 (1H, broad, Z/E forms), 9.80-10.00 (1H, broad, Z/E forms). LCMS t_{R} (min): 2.07. MS (APCI), m/z 434.04 [M+H]⁺. HPLC t_{R} (min): 16.25. Mₚ 149-150° C.

### 3. N-Furan-2-ylmethyl-6-propoxy-N′-(3-trifluoromethyl-phenyl)-[1,3,5]triazine-2,4-diamine

¹H-NMR (400 MHz, DMSO-D₆) δ_{H}: 0.95 (3H, t, J=7.5 Hz), 1.70 (2H, q, J=7.5 Hz), 4.22 (2H, broad), 4.50 (2H, broad), 6.25 (1H, broad), 6.38 (1H, broad), 7.30 (1H, d, J=8.5 Hz), 7.50 (1H, t, J=8.5 Hz), 7.54 (1H, s), 7.88 (1H, broad), 7.92-8.08 (1H, broad, Z/E forms), 8.15-8.38 (1H, broad, Z/E forms), 9.62-9.80 (1H, broad, Z/E forms). LCMS t_{R} (min): 2.07. MS (APCI), m/z 394.09 [M+H]⁺. HPLC t_{R} (min): 15.65. Mₚ 60-62° C.

### 4. 6-Cyclopropylmethoxy-N-furan-2-ylmethyl-N′-(3-trifluoromethyl-phenyl)-[1,3,5]triazine-2,4-diamine

¹H-NMR (400 MHz, DMSO-D₆) δ_{H}: 0.31 (2H, m), 0.55 (2H, m), 1.24 (1H, m), 4.12 (2H, broad), 4.50 (2H, broad), 6.25 (1H, broad), 6.40 (1H, broad), 7.30 (1H, d, J=8.5 Hz), 7.50 (1H, t, J=8.5 Hz), 7.55 (1H, s), 7.88 (1H, broad), 7.90-8.09 (1H, broad, Z/E forms), 8.16-8.40 (1H, broad, Z/E forms), 9.60-9.80 (1H, broad, Z/E forms). LCMS t_{R} (min): 2.04. MS (APCI), m/z 406.02 [M+H]⁺. HPLC t_{R} (min): 15.69. Mₚ 138-140° C.

### 5. 6-Allyloxy-N-furan-2-ylmethyl-N′-(3-trifluoromethyl-phenyl)-[1,3,5]triazine-2,4-diamine

Yield 490 mg, 66%. ¹H-NMR (400 MHz, DMSO-D₆) δ_{H}: 4.51 (2H, broad), 4.81 (2H, broad), 5.23 (1H, d, J=10.0 Hz), 5.38 (1H, d, J=16.0 Hz), 6.05 (1H, m), 6.25 (1H, broad), 6.39 (1H, broad), 7.30 (1H, d, J=8.5 Hz), 7.50 (1H, t, J=8.5 Hz), 7.55 (1H, s), 7.84-7.98 (1H, broad, Z/E forms), 8.00 (1H, broa), 8.11-8.36 (1H, broad, Z/E forms), 9.65-9.88 (1H, broad, Z/E forms). LCMS t_{R} (min): 2.00. MS (ARCI), m/z 391.90 [M+H]⁺. HPLC t_{R} (min): 15.38. Mₚ 235-23° C.

### 6. 4-[(Furan-2-ylmethyl)-amino]-6-(4-hydroxy-phenylamino)-[1,3,5]triazin-2-ol

A mixture of compound I-45 (320 mg, 1 mmol), sodium hydroxide (0.12 g, 3 mmol) and water (10 mL) was stirred at refluxing for 4 hours, cooled to room temperature and neutralized with concentrated HCl aqueous solution to reach pH 2. The formed solid was collected by filtration, washed with water and cold ethanol to give the compound (105 mg, 35%). ¹H-NMR (400 MHz, DMSO-D₆) δ_{H}: 3.20 (1H, broad), 4.45 (2H, broad), 6.27 (1H, broad), 6.39 (1H, broad), 6.73 (2H, d, J=8.6 Hz), 7.36 (2H, d, J=8.6 Hz), 7.57 (1H, broad), 7.80-8.30 (1H, broad), 9.15-9.40 (1H, broad). LCMS t_{R} (min) 1.22. MS (APCI), m/z 299.64 [M+H]⁺.

### 7. 4-[4-[(Furan-2-ylmethyl)-amino]-6-(4-hydroxy-phenoxy)[1,3,5]triazin-2-ylamino]-phenol

A mixture of I-45 (250 mg, 0.78 mmol), p-hydroxyphenol (257 mg, 2.34 mmol), potassium carbonate (414 mg, 3 mmol) and DMSO (200 μl) was stirred for 4 hours at 180° C., diluted with water (20 mL) and extracted with ethyl acetate (2×10 mL). The combined organic phases were concentrated at reduced pressure. Purification by column chromatography (silica gel, ethyl acetate hexane) gave the compound (40 mg, 13%). ¹H-NMR (400 MHz, DMSO-D₆) δ_{H}: 4.30-4.50 (2H, broad, Z/E forms), 6.05-6.30 (1H, broad, Z/E forms), 6.35 (1H, t, J=3.6 Hz), 6.60 (2H, broad), 6.74 (2H, d, J=8.5 Hz), 6.94 (2H, d, J=8.5 Hz), 7.35 (2H, broad), 7.52 (1H, d, J=1.8 Hz), 7.73 (1H, broad), 8.92 (1H, s), 8.88-9.20 (1H, broad, Z/E forms), 9.20 (1H, s). LCMS t_{R} 1.44 (min). MS (APCI), m/z 391.98 [M+H]⁺. Mₚ 80-83° C.

### 8. N-Furan-2-ylmethyl-6-(pyridin-4-ylmethoxy)-N′-(3-trifluoromethyl-phenyl)-[1,3,5]triazine-2,4-diamine

To a solution of pyridin-4-yl-methanol (90 mg, 0.81 mmol) in THF (2 mL) NaH (60% in oil, 35 mg, 0.89 mmol) was added at 0° C. The obtained suspension was stirred at 0° C. for 15 min. Then a solution of N-furan-2-ylmethyl-6-(chloro)-N′-(3-trifluoromethyl-phenyl)-[1,3,5]triazine-2,4-diamine (300 mg, 0.81 mmol) in THF (2 mL) was added to the obtained suspension at 0° C. The final reaction mixture was stirred at 0° C. for 30 minutes, at room temperature for 1 hour and at 50° C. for 3.5 hours, cooled to room temperature, concentrated at reduced pressure and purified by preparative TLC to give the compound (47 mg, 13%). ¹H-NMR (400 MHz, DMSO-D₆) δ_{H}: 4.51 (2H, broad), 5.43 (2H, s), 6.22 (1H, broad), 6.38 (1H, broad), 7.30 (1H, d, J=8.5 Hz), 7.38 (2H, d, J=5.0 Hz), 7.48 (1H, t, J=8.5 Hz), 7.52 (1H, s), 7.85-8.01 (1H, broad, Z/E forms), 8.05 (1H, broad), 8.18-8.31 (1H, broad, Z/E forms), 8.56 (2H, d, J=5.0 Hz), 9.68-9.88 (1H, broad, Z/E forms). LCMS t_{R} (min): 1.61. MS (APCI), m/z 443.01 [M+H]⁺. HPLC t_{R} (min): 10.06. Mₚ 124-126° C.

### 9. 6. 4-(4-Fluoro-benzylamino)-6-(3-trifluoromethyl-phenylamino)-[1,3,5]triazin-2-ol

A mixture of compound 5 (200 mg, 0.515 mmol), sulfuric acid (6 mL) and water (6 mL) was stirred at 125° C. for 4 hours and cooled to room temperature. The formed precipitate was collected by filtration, washed with water and ethyl acetate and dried giving the compound. Yield 106 mg, 54%. ¹H-NMR (400 MHz, DMSO-D₆) δ_{H}: 4.15 (1H, broad), 4.50 (2H, broad, Z/E forms), 7.12 (2H, broad, Z/E forms), 7.32 (2H, broad, Z/E forms), 7.43 (1H, d, J=8.5 Hz), 7.53 (1H, t, J=8.5 Hz), 7.78 (1H, broad), 8.04 (2H, broad), 9.89 (1H, broad). MW 379.32. LCMS t_{R} (min): 1.65. MS (APCI), m/z 380.08 [M+H]⁺. HPLC t_{R} (min): 11.16. Mₚ 266-268° C.

### Preparation of C,N,N-Triazines in Table 7

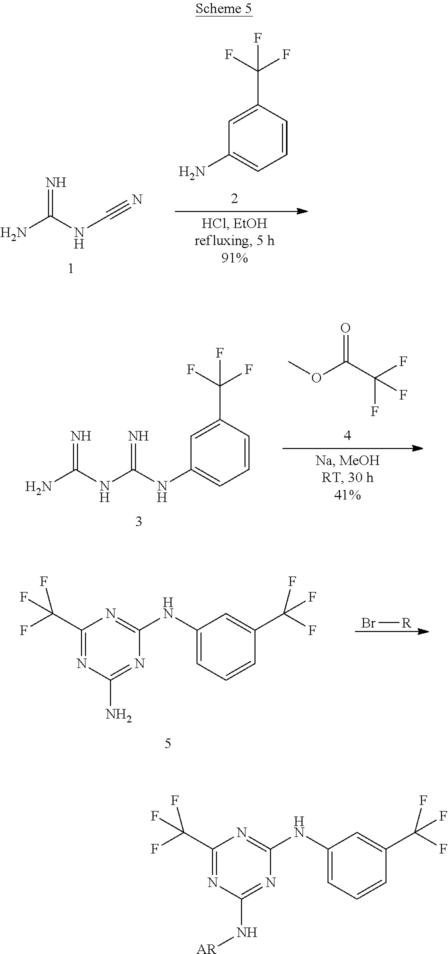

### Generic Procedure for Synthesis of CF₃,N,N-triazines

Compounds with trifluoromethyl group directly connected to the triazine core were prepared starting from acyclic precursors. Condensation of bis-guanidine 3 with TFA methyl ester gave the triazine derivative 5 in 41% yield. Further derivatization of amine function in 5 was performed by the alkylation with various aryl bromides and alkyl halides or acylation followed by amide reduction affording the final products.

### Preparation of 6-Trifluoromethyl-N-(3-trifluoromethyl-phenyl)-[1,3,5]triazine-2,4-diamine (5)

A mixture of compound 1 (2.58 g, 16.01 mmol), m-trifluoroaniline (2) (1.481 g, 17.61 mmol), concentrated aqueous solution of HCl (1.383 mL, 16.10 mmol) and ethanol (10 mL) was stirred at refluxing for 5 hours, cooled to 5° C., concentrated at reduced pressure and dried giving compound 3 (4.112 g, 91%) used on the next stage without additional purification. Sodium (165 mg, 7.15 mmol) was dissolved in methanol (10 mL). Then compound 3 (1.00 g, 3.55 mmol) was added to the obtained solution of sodium methoxide. The mixture was stirred at room temperature for 1.5 hours. Then 2,2,2-trifluoroacetate (4) (445 mg, 3.55 mmol) was added. The resulting mixture was stirred at room temperature for 30 hours, diluted with water and extracted with ethyl acetate. The combined organic phases were dried over sodium sulfate and concentrated at reduced pressure. The residue was purified by column chromatography (silica gel, dichloromethane/ethanol 20/1) giving compound 5. Yield 469 mg, 41%.

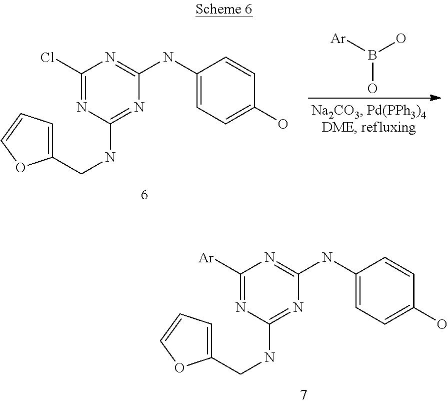

### Generic Procedure for Synthesis of Ar,N,N-triazines

Suzuki coupling starting from monochloro-1,3,5-triazine led to the formation of the derivative aryl substituted triazine derivatives. A mixture of monochloro-1,3,5-triazine (1.0 mmol), boronic acid (1.0 mmol), Pd(PPh₃)₄ (120 mg, 0.1 mol, 10 mol %), Na₂CO₃ (424 mg, 4.0 mmol), dimethoxy ethane (3 mL) and water (3 mL) was stirred at refluxing for 3 hours, cooled to room temperature, filtered through a pad of Celite, extracted with ethyl acetate (2×20 mL). The combined organic phases were combined, dried over sodium sulfate and concentrated. Purification by column chromatography gave a final compound.

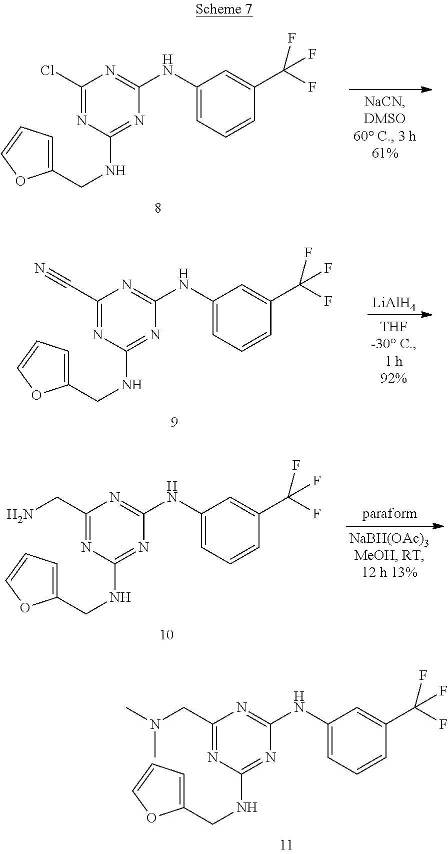

### Generic Procedure for Synthesis of Cyanotriazine and Related Derivatives

Preparation of the Cyanotriazine and Related Derivatives was Started from conversion of mono-chloros triazine 8 to the nitrile triazine 9, and the nitro group was reduced to aminomethyl group in 10 by the reaction with LiAlH₄ at −30° C. The primary amine 10 was further converted into N,N-alkylated derivative 11 by the reductive amination.

TABLE 7

| En | Structure | Name0 | MW | Formula |
|---|---|---|---|---|
| 1 | 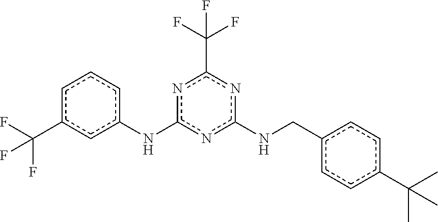 | N-(4-tert-butylbenzyl)-6- (trifIuoromethyl)-N′-[3- (trifluoromethyl)phenyl]-1,3,5- triazine-2,4-diamine | 469.4 | C22H21F6N5 |
| 2 | 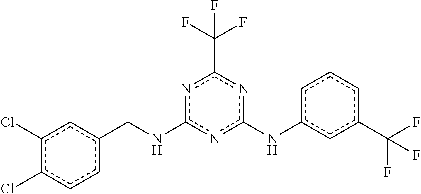 | N-(3,4-dichlorobenzyl)-6- (trifluoromethyl)-N′-[3- (trifluoromethyl)phenyl]-1,3,5- triazine-2,4-diamine | 482.2 | C18H11Cl2F6N5 |
| 3 | 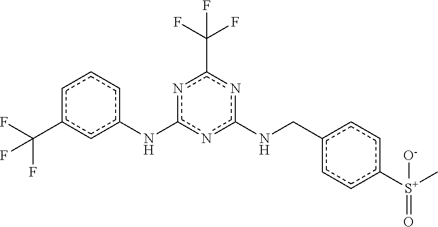 | Structure contains group(s) not supported in current version! | 491.4 | C19H15F6N5O2S |
| 4 | 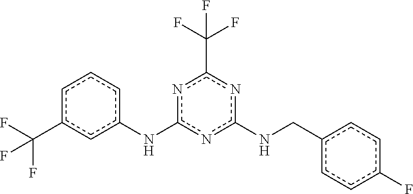 | N-(4-fluorobenzyl)-6- (trifluoromethyl)-N′-[3- (trifluoromethyl)phenyl]-1,3,5- triazine-2,4-diamine | 431.3 | C18H12F7N5 |
| 5 | 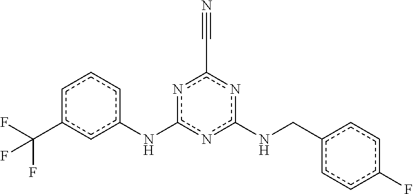 | 4-[(4-fluorobenzyl)amino]-6-{[3- (trifluoromethyl)phenyl]amino}- 1,3,5-triazine-2-carbonitrile | 388.3 | C18H12F4N6 |
| 6 | 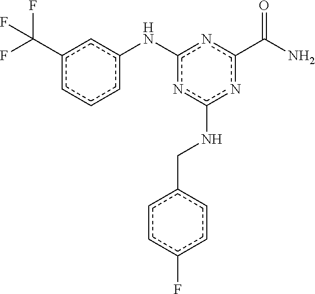 | 4-[(4-fluorobenzyl)amino]-6-{[3- (trifluoromethyl)phenyl]amino}- 1,3,5-triazine-2-carboxamide | 406.3 | C18H14F4N6O |
| 7 | 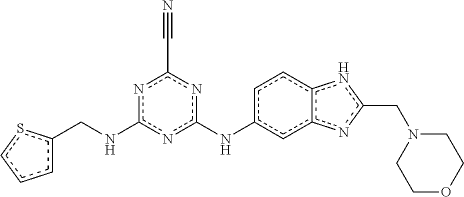 | 4-{[2-(morpholin-4-ylmethyl)-1H- benzimidazol-5-yl]amino}-6- [(thiophen-2-ylmethyl)amino]- 1,3,5-triazine-2-carbonitrile | 447.5 | C12H21N9OS |
| 8 | 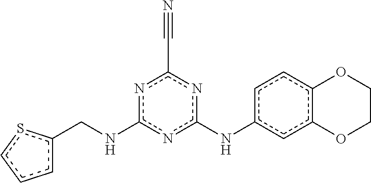 | 4-(2,3-dihydro-1,4-benzodioxin- 6-ylamino)-6-[(thiophen-2- ylmethyl)amino]-1,3,5-triazine-2- carbonitrile | 366.4 | C17H14N6O2S |
| 9 | 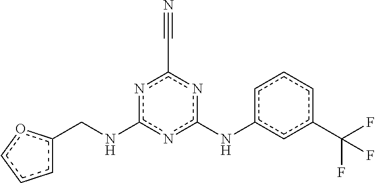 | 4-[(furan-2-ylmethyl)amino]-6- {[3- (trifluoromethyl)phenyl]amino}- 1,3,5-triazine-2-carbonitrile | 360.3 | C16H11F3N6O |
| 10 | 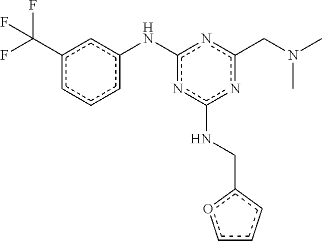 | 6-[(dimethylamino)methyl]-N- (furan-2-ylmethyl)-N′-[3- (trifluoromethyl)phenyl]-1,3,5- triazine-2,4-diamine | 392.4 | C18H19F3N6O |
| 11 | 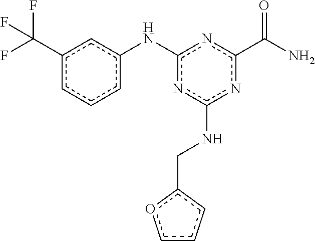 | 4-[(furan-2-ylmethyl)amino]-6- {[3- (trifluoromethyl)phenyl]amino}- 1,3,5-triazine-2-carboxamide | 378.3 | C16H13F3N6O2 |
| 12 | 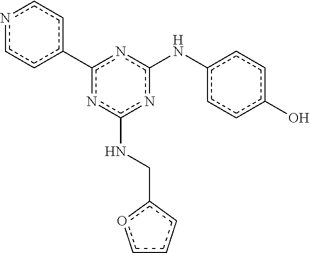 | 4-({4-[(furan-2-ylmethyl)amino]- 6-(pyridin-4-yl)-1,3,5-triazin-2- yl}amino)phenol | 360.4 | C19H16N6O2 |
| 13 | 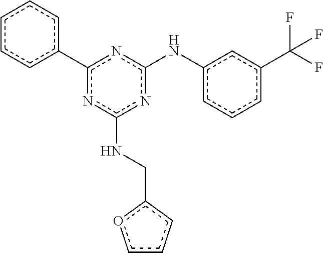 | N-(furan-2-ylmethyl)-6-phenyl-N′- [3-(trifluoromethyl)phenyl]-1,3,5- triazine-2,4-diamine | 411.4 | C21H16F3N5O |

### Procedures and Analytical Data for Compounds in Table 7.

### 1. 6. N-(4-tert-Butyl-benzyl)-6-trifluoromethyl-N′-(3-trifluoromethyl-phenyl)-[1,3,5]triazine-2,4-diamine

To a mixture of compound 5 (200 mg, 0.62 mmol) and KOH (139 mg, 1.24 mmol) in DMSO (2 mL) p-tert-butylbenzyl bromide (211 mg, 0.93 mmol) was added. The resulting mixture was stirred at room temperature for 40 minutes, diluted with water and extracted with ethyl acetate. The combined organic phases were washed with water, brine, dried over sodium sulfate and concentrated. The residue was purified by prepTLC (dichloromethane/hexane 1/3) giving the compound. Yield 172 mg, 59%. ¹H-NMR (400 MHz, DMSO-D₆) δ_{H}: 1.20 (9H, s), 5.21 (2H, broad), 7.14 (2H, d, J=8.5 Hz), 7.28 (2H, d, J=8.5 Hz), 7.56 (5H, broad), 7.65 (1H, broad). MW 469.44. LCMS t_{R} (min): 2.26. MS (APCI), m/z 470.19 [M+H]⁺. HPLC t_{R} (min): 19.95. M_{P} 53-55° C.

### 2. N-(3,4-Dichloro-benzyl)-6-trifluoromethyl-N′-(3-trifluoromethyl-phenyl)-[1,3,5]triazine-2,4-diamine

¹H-NMR (400 MHz, DMSO-D₆) δ_{H}: 5.25 (2H, broad), 7.25 (1H, d, J=8.5 Hz), 7.51 (1H, broad d, J=8.5 Hz), 7.56 (4H, broad), 7.69 (2H, broad). MW 482.22. LCMS t_{R} (min): 2.17. MS (APCI), m/z 482.12, 484.10 [M+H]⁺. HPLC t_{R} (min): 18.19. M_{P} 58-60° C.

### 3. N-(4-Methanesulfonyl-benzyl)-6-trifluoromethyl-N′-(3-trifluoromethyl-phenyl)-[1,3,5]triazine-2,4-diamine

¹H-NMR (400 MHz, DMSO-D₆) δ_{H}: 3.14 (3H, s), 5.38 (2H, broad), 7.56 (4H, broad m), 7.74 (2H, broad), 7.82 (2H, d, J=8.5 Hz). MW 491.42. LCMS t_{R} (min): 1.94. MS (APCI), m/z 492.04 [M+H]⁺. HPLC t_{R} (min): 14.40. M_{P} 156-158° C.

### 4. N-(4-Fluoro-benzyl)-6-trifluoromethyl-N′-(3-trifluoromethyl-phenyl)-[1,3,5]triazine-2,4-diamine

To a mixture of compound 5 (200 mg, 0.62 mmol) and KOH (139 mg, 1.24 mmol) in DMSO (2 mL) p-fluorobenzyl bromide (234 mg, 1.24 mmol) was added. The resulting mixture was stirred at room temperature for 40 minutes, diluted with water and extracted with ethyl acetate. The combined organic phases were washed with water, brine, dried over sodium sulfate and concentrated. The residue was purified by prepTLC (dichloromethane/acetone 30/1) giving the compound. Yield 110 mg, 41%. ¹H-NMR (400 MHz, DMSO-D₆) δ_{H}: 5.25 (2H, broad), 7.07 (2H, m), 7.27 (2H, broad), 7.52 (1H, broad), 7.55 (3H, broad), 7.61 (1H, broad), 7.68 (1H, broad). MW 431.32. LCMS t_{R} (min): 2.04. MS (APCI), m/z 432.04 [M+H]⁺. HPLC t_{R} (min): 16.59. M_{P} 106-107° C.

### 5. 4-[(4-fluorobenzyl)amino]-6-{[3-(trifluoromethyl)phenyl]amino}-1,3,5-triazine-2-carbonitrile

MS (APCI), m/z 389.3 [M+H]⁺.

### 6. 4-[(4-fluorobenzyl)amino]-6-{[3-(trifluoromethyl)phenyl]amino}-1,3,5-triazine-2-carboxamide

Basic hydrolysis with a solution of NaOH in ethanol in the presence of 30% H₂O₂ at room temperature for 1.5 hour gave the desired amide in 47% yield. MS (APCI), m/z 407.3 [M+H]⁺.

### 7. 4-(2-Morpholin-4-ylmethyl-1H-benzoimidazol-5-ylamino)-6-[(thiophen-2-ylmethyl)-amino]-[1,3,5]triazine-2-carbonitrile

A mixture of 2-chloro-4-(2-Morpholin-4-ylmethyl-1H-benzoimidazol-5-ylamino)-6-[(thiophen-2-ylmethyl)-amino]-[1,3,5]triazine-(315 mg, 0.69 mmol), NaCN (169 mg, 3.45 mmol) and DMSO (3 mL) was stirred at 80° C. for 12 hours, cooled down to room temperature, diluted with water and extracted with dichloromethane. The combined organic phases were dried over sodium sulfate and concentrated at reduced pressure. Purification by preparative TLC (5% EtOH/dichloromethane) gave the compound. Yield 13 mg, 4%. ¹H-NMR (400 MHz, CDCl₃) δ_{H}: 2.60 (4H, m), 3.77 (4H, m), 3.83 (2H, s), 4.82 (2H, broad), 5.70-6.02 (1H, broad, Z/E forms), 7.01 (2H, m), 7.24 (1H, broad), 7.29 (1H, broad), 7.36-7.69 (2H, broad, Z/E forms), 7.99 (1H, broad), 9.58 (1H, broad). LCMS t_{R} (min): 1.44. MS (APCI), m/z 448.04 [M+H]⁺. HPLC t_{R} (min): 9.64.

### 8. 4-(2,3-Dihydro-benzo[1,4]dioxin-6-ylamino)-6-[(thiophen-2-ylmethyl)-amino]-[1,3,5]triazine-2-carbonitrile

¹H-NMR (400 MHz, DMSO-D₆) δ_{H}: 4.21 (4H, m), 4.68 (2H, d, J=7.5 Hz), 6.78 (1H, d, J=8.5 Hz), 6.97 (1H, broad), 7.03 (1H, broad), 7.08 (1H, broad, Z/E forms), 7.29 (1H, broad), 7.38 (1H, broad, Z/E forms), 8.60-8.85 (1H, broad, Z/E forms), 9.80-10.09 (1H, broad, Z/E forms). LCMS t_{R} (min): 1.88. MS (APCI), m/z 367.00 [M+H]⁺. HPLC t_{R} (min): 14.15. Mₚ 184-186° C.

### 9. 4-[(Furan-2-ylmethyl)-amino]-6-(3-trifluoromethyl-phenylamino)-[1,3,5]triazine-2-carbonitrile (19)

A mixture of compound 8 (4.50 g, 12.17 mmol), NaCN (2.98 g, 60.85 mmol) and DMSO (40 mL) was stirred at 60° C. for 3 hours (LCMS control of the reaction), cooled to room temperature and diluted with water. The formed solid was collected by filtration and purified by column chromatography (silica gel, dichloromethane) to give the compound.

Yield 2.67 g, 61%. ¹H-NMR (400 MHz, DMSO-D₆) δ_{H}: 4.53 (2H, broad), 6.22-6.37 (1H, broad, BE forms), 6.37-6.44 (1H, broad, Z/E forms), 7.40 (1H, d, J=8.5 Hz), 7.58 (2H, m), 7.88 (1H, d, J=8.5 Hz), 8.02-8.30 (1H, broad, BE forms), 8.68-8.90 (1H, broad, BE forms), 10.30-10.51 (1H, broad, Z/E forms). LCMS t_{R} (min): 1.96. MS (APCI), m/z 360.68 [M+H]⁺. HPLC t_{R} (min): 15.55. Mₚ 157-159° C.

### 10. 6-Dimethylaminomethyl-N-furan-2-ylmethyl-N′-(3-trifluoromethyl-phenyl)-[1,3,5]triazine-2,4-diamine

To a suspension of LiAlH₄ (1.41 g, 37.05 mmol) in THF (50 mL) 6-nitrile-N-furan-2-ylmethyl-N′-(3-trifluoromethyl-phenyl)-[1,3,5]triazine-2,4-diamine (2.67 g, 7.41 mmol) was added slowly portionwise at −30° C. The reaction mixture was stirred at the same temperature for 1 hour. Then ethanol (20 mL) was added dropwise to the reaction mixture at −30° C. following by addition of 15% aqueous solution of KOH (160 mL). The formed solid was filtered off and washed by ethylacetate. The combined filtrates were washed with water, brine and dried under sodium sulfate yielding amino derivative (2.50 g, 92%).

A mixture of the amino derivative (1.00 g, 2.78 mmol), paraform (574 mg, 6.38 mmol), sodium triacetoxyboronhydride (1.76 g, 8.33 mmol) in methanol (12 mL) was stirred at room temperature for 12 hours, diluted with saturated aqueous sodium hydrocarbonate solution and water and extracted with ethyl acetate. The combined organic phases were washed with water, brine, dried over sodium sulfate and concentrated at reduced pressure. Purification by column chromatography on silica gel (ethanol/dichloromethane) gave the compound. Yield 75 mg, 13%. ¹H-NMR (400 MHz, DMSO-D₆) δ_{H}: 2.28 (6H, s), 3.35 (2H, s), 4.50 (2H, broad), 6.22 (1H, broad), 6.34 (1H, broad), 7.28 (1H, d, J=8.5 Hz), 7.49 (1H, t, J=8.5 Hz), 7.53 (1H, s), 7.89 (1H, d, J=8.5 Hz), 8.10 (1H, broad), 8.15-8.40 (1H, broad, Z/E forms), 9.75-9.95 (1H, broad, Z/E forms). LCMS t_{R} (min): 1.52. MS (APCI), m/z 392.83 [M+H]⁺. HPLC t_{R} (min): 11.11. Mₚ 133-135° C.

### 11. 4-[(Furan-2-ylmethyl)-amino]-6-(3-trifluoromethyl-phenylamino)-[1,3,5]triazine-2-carboxylic acid amide

H₂O₂ (30%, 1 mL) was added dropwise to a mixture of compound 21 (30 mg, 0.08 mmol), K₂CO₃ (31 mg, 0.22 mmol) and DMSO (0.5 mL). The obtained reaction mixture was stirred at room temperature for 1 hour, diluted with water. The formed solid was collected by filtration, washed with water and dried to give compound 22. Yield 22 mg, 70%. ¹H-NMR (400 MHz, DMSO-D₆) δ_{H}: 4.51-4.68 (2H, broad, Z/E forms), 6.21-6.39 (1H, broad, Z/E forms), 6.32-6.42 (1H, broad, Z/E forms), 7.32 (1H, d, J=8.5 Hz), 7.53 (2H, m), 7.62 (1H, broad), 7.92 (1H, broad), 8.12-8.25 (2H, broad, Z/E forms), 8.30-8.45 (1H, broad, Z/E forms), 10.12-10.26 (1H, broad, Z/E forms). LCMS t_{R} (min): 1.73. MS (APCI), m/z 379.08 [M+H]⁺. HPLC t_{R} (min): 12.44. Mₚ 258-260° C.

### 12. 4-{4-[(Furan-2-ylmethyl)-amino]-6-pyridin-4-yl-[1,3,5]triazin-2-ylamino}-phenol

¹H-NMR (400 MHz, DMSO-D₆) δ_{H}: 4.45-4.65 (2H, broad, Z/E forms), 6.20-6.35 (1H, broad, Z/E forms), 6.37 (1H, s), 6.70 (2H, d, J=8.5 Hz), 7.48 (2H, broad, Z/E forms), 7.52 (1H, s), 7.60-8.00 (1H, broad, Z/E forms), 8.00-8.20 (2H, broad, Z/E forms), 8.72 (2H, d, J=5 Hz), 9.02 (1H, s), 9.20-9.50 (1H, broad, Z/E forms). LCMS t_{R} 1.36 (min). MS (APCI), m/z 361.13 [M+H]⁺. Mₚ 87-89° C.

### 13. N-Furan-2-ylmethyl-6-phenyl-N′-(3-trifluoromethyl-phenyl)-[1,3,5]triazine-2,4-diamine

A mixture of compound 8 (200 mg, 0.54 mmol), phenyl boronic acid (99 mg, 0.81 mmol), Pd(PPh₃)₄ (17 mg, 0.015 mol, 3 mol %), K₂CO₃ (140 mg, 1.0 mmol), dimethoxy ethane (4 mL) and water (4 mL) was stirred at 80° C. for 6.5 hours, cooled to room temperature, concentrated at reduced pressure and diluted with THF. The formed solid was filtered off, washed with THF and combined filtrates were concentrated. Purification by preparative HPLC (acetonitrile/water) gave the compound. Yield 70 mg, 32%. ¹H-NMR (400 MHz, DMSO-D₆) δ_{H}: 4.52-4.72 (2H, broad, Z/E forms), 6.25-6.36 (1H, broad, Z/E forms), 6.38 (1H, broad), 7.32 (1H, d, J=8.5 Hz), 7.55 (5H, m), 7.92-8.08 (1H, broad, Z/E forms), 8.09-8.22 (1H, broad, Z/E forms), 8.34 (2H, broad), 8.35-8.54 (1H, broad, Z/E forms), 9.80-9.99 (1H, broad, Z/E forms). LCMS t_{R} 2.20 (min). MS (APCI), m/z 412.11 [M+H]⁺. HPLC t_{R} (min): 16.88. Mₚ 133-134° C.

### Prepare C,N,N-triazines in Table 8

The “R2-variation” compounds containing C-linker at 2-position were synthesized through three step reaction sequence as shown in Scheme 8:

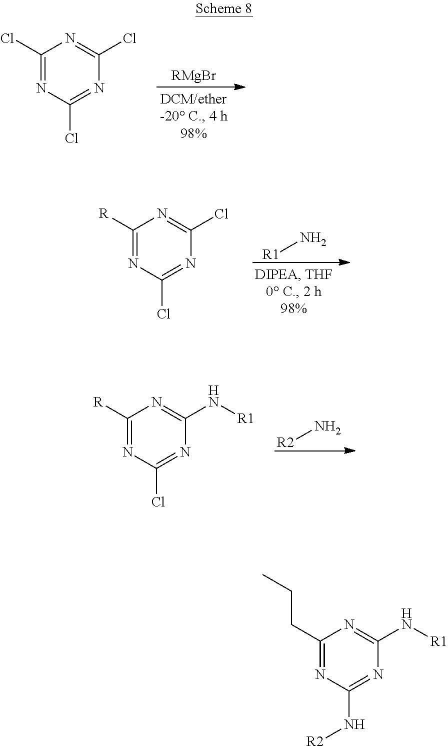

Introduction of n-propyl and alkylaryl fragment via reaction of the corresponding Grinard reagent on the second and third stages gave complex mixtures of byproducts. The reaction of 2,4,6-trichlorotriazine with magnesium-derivative at reduced temperature gave rise to the dichloro-substituted compound in quantitative yield. Then, the next chlorine atom was replaced by amino-substituted moiety that resulted in the key intermediates in good yields. The final compounds were obtained by reacting with secondary amine or aniline in the presence of weak bases (triethylamine or potassium carbonate) at heating in most cases in good yields.

TABLE 8

| En | Structure | IUPAC Name | MW | Formula |
|---|---|---|---|---|
| 1 | 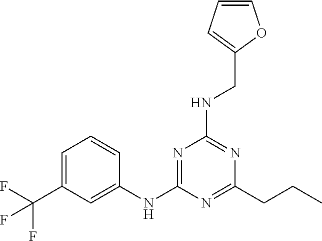 | N-(furan-2-ylmethyl)-6-propyl-N′-[3- (trifluoromethyl)phenyl]-1,3,5- triazine-2,4-diamine | 377.4 | C18H18F3N5O |
| 2 | 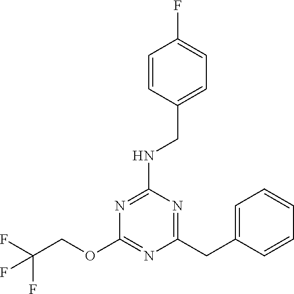 | 4-benzyl-N-(4-fluorobenzyl)-6-(2,2,2- trifluoroethoxy)-1,3,5-triazin-2-amine | 392.4 | C19H16F4N4O |
| 3 | 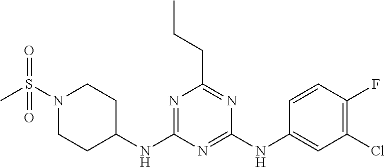 | N-(3-chloro-4-fluorophenyl)-N′-[1- (methylsulfonyl)piperidin-4-yl]-6- propyl-1,3,5-triazine-2,4-diamine | 444.0 | C18H24ClFN6O2S |
| 4 | 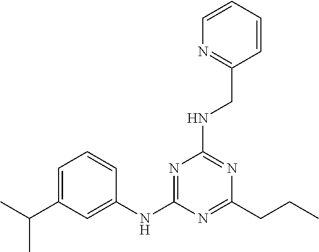 | N-[3-(propan-2-yl)phenyl]-6-propyl- N′-(pyridin-2-ylmethyl)-1,3,5-triazine- 2,4-diamine | 362.5 | C21H26N6 |
| 5 | 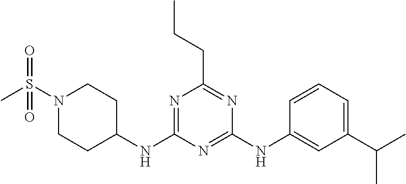 | N-[1-(methylsulfonyl)piperidin-4-yl]- N′-[3-(propan-2-yl)phenyl]-6-propyl- 1,3,5-triazine-2,4-diamine | 433.6 | C21H32N6O2S |
| 6 | 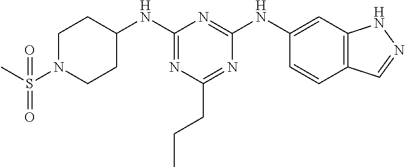 | N-(1H-indazol-6-yl)-N′-[1- (methylsulfonyl)piperidin-4-yl]-6- propyl-1,3,5-triazine-2,4-diamine | 431.5 | C19H26N8O2S |
| 7 | 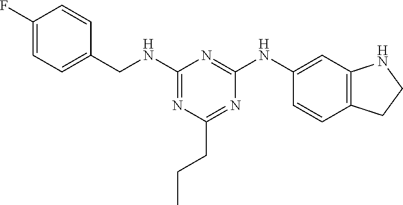 | N-(2,3-dihydro-1H-indol-6-yl)-N′-(4- fluorobenzyl)-6-propyl-1,3,5-triazine- 2,4-diamine | 378.4 | C21H23FN6 |
| 8 | 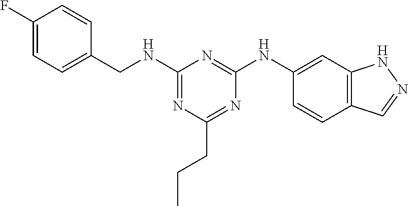 | N-(4-fluorobenzyl)-N′-(1H-indazol-6- yl)-6-propyl-1,3,5-triazine-2,4- diamine | 377.4 | C20H20FN7 |
| 9 | 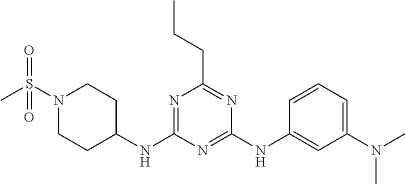 | N-[3-dimethylamino)phenyl]-N′-[1- (methylsulfonyl)piperidin-4-yl]-6- propyl-1,3,5-triazine-2,4-diamine | 434.6 | C20H31N7O2S |
| 10 | 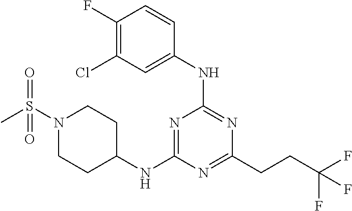 | N-(3-chloro-4-fluorophenyl)-N′-[1- (methylsulfonyl)piperidin-4-yl]-6- (3,3,3-trifluoropropyl)-1,3,5-triazine- 2,4-diamine | 497.9 | C18H21ClF4N6O2S |
| 11 | 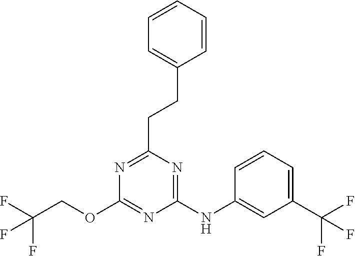 | 4-(2-phenylethyl)-6-(2,2,2- trifluoroethoxy)-N-[3- (trifluoromethyl)phenyl]-1,3,5-triazin- 2-amine | 442.4 | C20H16F6N4O |

### Procedures and Analytical Data for Compounds in Table 8.

### 1. N-Furan-2-ylmethyl-6-propyl-N′-(3-trifluoromethyl-phenyl)-[1,3,5]triazine-2,4-diamine

A mixture of compound (3-trifluoromethyl-phenyl)-(4-chloro-6-propyl-[1,3,5]triazin-2-yl)-amine (465 mg, 1.5 mmol), furfurylamine (0.137 mL, 1.5 mmol), DIPEA (0.267 mL, 1.5 mL) and acetonitrile (6 mL) was stirred at room temperature for 3 hours and at 50° C. for 1 h, cooled to room temperature, diluted with water and extracted with ethyl acetate. The combined organic phases were washed with brine, dried over sodium sulfate and concentrated. Purification by column chromatography (silica gel, dichloromethane), preparative TLC (ethyl acetate/hexane) gave desired product. Yield 53 mg, 10%. ¹H-NMR (400 MHz, DMSO-D₆) δ_{H}: 0.93 (3H, t, J=7.5 Hz), 1.72 (2H, m), 2.45 (2H, broad), 4.52 (2H, broad), 6.23 (1H, broad), 6.39 (1H, broad), 7.28 (1H, d, J=8.5 Hz), 7.48 (1H, t, J=8.5 Hz), 7.53 (1H, s), 7.80-7.91 (1H, broad, Z/E forms), 7.91-8.10 (1H, broad, Z/E forms), 8.19-8.42 (1H, broad, Z/E forms), 9.65-9.82 (1H, broad, Z/E forms). LCMS t_{R} (min): 1.91. MS (APCI), m/z 378.08 [M+H]⁺. HPLC t_{R} (min): 12.93. Mₚ 110-112° C.

### 2. [4-Benzyl-6-(2,2,2-trifluoro-ethoxy)-[1,3,5]triazin-2-yl]-(4-fluoro-benzyl)-amine

Yield 152 mg, 21%. ¹H-NMR (400 MHz, DMSO-D₆) δ_{H}: 3.81 (2H, s), 4.45 (2H, broad, Z/E forms), 4.90 (2H, broad q, J=7.5 Hz, Z/E forms), 7.08 (2H, superposition of two d/d, J=8.5/8.0 Hz), 7.25 (6H, broad), 7.32 (1H, t, J=8.5 Hz), 8.52-8.70 (1H, two broad signals, Z/E forms). MW 392.36. LCMS t_{R} (min): 2.04. MS (APCI), m/z 393.09 [M+H]⁺. HPLC t_{R} (min): 16.28. M_{P} 89-91° C.

### 3. N-(3-Chloro-4-fluoro-phenyl)-N′-(1-methanesulfonyl-piperidin-4-yl)-6-propyl-[1,3,5]triazine-2,4-diamine

A mixture of 3-chloro-4-fluoro-phenylamine (674 mg, 4.63 mol) and triethylamine (468 mg, 0.651 mL, 4.63 mol) in THF (8 mL) was added in portions to 2,4-dichloro-6-propyl-[1,3,5]triazine (890 mg, 4.63 mmol) dissolved in THF (10 mL) at 0° C. for 2.5 hours. Then the mixture was stirred at 0° C. for 3 hours. Then, the mixture was diluted with water and extracted with ethyl acetate, washed with water, with brine, dried over sodium sulfate and concentrated. Purification by column chromatography on silica gel (DCM) gave 3-Chloro-4-fluoro-phenyl)-(4-chloro-6-propyl-[1,3,5]triazin-2-yl)-amine. Yield 702 mg, 50%.

A mixture of 3-Chloro-4-fluoro-phenyl)-(4-chloro-6-propyl-[1,3,5]triazin-2-yl)-amine (200 mg, 0.66 mmol), 1-methanesulfonyl-piperidin-4-ylamine hydrochloride (150 mg, 0.73 mmol) and triethylamine (148 mg, 0.205 mL, 1.46 mmol) dissolved in acetonitrile (5 mL) was stirred for 7 hours at room temperature and left overnight. The reaction was monitored by TLC. When the reaction was over, the reaction mixture was poured into water (30 mL) and extracted with ethyl acetate. The combined organic phases were washed with water, and a target compound was extracted with EtOAc (2×30 mL). The organic phases were combined, washed with water, with brine, dried over sodium sulfate and concentrated. The residue (214 mg) was purified by flash-chromatography on silica gel (ethyl acetate/hexane, 1/1) that gave the final compound as white solid. Yield 124 mg, 42%. ¹H-NMR (400 MHz, DMSO-D₆) δ_{H}: 0.93 (3H, superposition of two t, J=7.5 Hz, Z/E forms), 1.60 (2H, m), 1.70 (2H, m), 1.94 (2H, m), 2.42 (2H, broad), 2.88 (5H, superposition of s and m), 3.60 (2H, broad), 3.90 (1H, broad), 7.28 (1H, broad), 7.34-7.75 (1H, two broad peaks, Z/E forms), 7.60 (1H, broad peak), 8.02-8.16 (1H, two broad peaks. Z/E forms), 9.48-9.62 (1H, two broad peaks. Z/E forms). MW 442.94. LCMS t_{R} (min): 1.73. MS (APCI), m/z 443.10, 445.10 [M+H]⁺. HPLC t_{R} (min): 11.27. Mp 86-87° C.

### 4. N-(3-Isopropyl-phenyl)-6-propyl-N′-pyridin-2-ylmethyl-[1,3,5]triazine-2,4-diamine

To a solution of 2,4-dichloro-6-propyl-[1,3,5]triazine (0.768 g, 4.0 mmol) and DIPEA (0.517, 4.0 mmol) in THF (3 mL) a solution of 3-isopropyl-phenylamine (0.54 g, 4 mmol) in THF (3 mL) was added slowly dropwise at 15-20° C. The resulting mixture was stirred at 20° C. for 0.5 hour (TLC control), diluted with water and ether. The organic phase was dried over Na₂SO₄ and concentrated. Purification by column chromatography on silica gel (hexane/ethyl acetate, 10/1) gave (4-Chloro-6-propyl-[1,3,5]triazin-2-yl)-(3-isopropyl-phenyl)-amine. Yield 1.1 g, 95%.

A mixture of (4-Chloro-6-propyl-[1,3,5]triazin-2-yl)-(3-isopropyl-phenyl)-amine (0.2 g, 0.69 mmol), C-pyridin-2-yl-methylamine (0.76 g, 0.7 mmol) and powdered K₂CO₃ (0.191 g, 1.38 mmol) in DMSO (1.5 mL) was stirred for 1.5 hours at 80° C. When the reaction was over according to TLC, the mixture was cooled, diluted with water, and extracted with dichloroethane. Extract was concentrated at the temperature below 40° C., and the residue was purified by column chromatography on silica gel (ethyl acetate/hexane, 1/1) that gave the final compound. Yield 224 mg, 90%. ¹H-NMR (400 MHz, DMSO-D₆) δ_{H}: 0.84-0.95 (3H, two broad peaks. Z/E forms), 1.09-1.21 (6H, two broad peaks, Z/E forms), 1.64-1.75 (2H, two broad peaks, Z/E forms), 2.42 (2H, broad), 2.70-2.86 (1H, two broad peaks. Z/E forms), 4.63 (2H, d, J=7.5 Hz), 6.77-6.83 (1H, two broad peaks. Z/E forms), 7.06-7.18 (1H, two broad peaks. Z/E forms), 7.22 (1H, broad), 7.30 (2H, broad), 7.66 (1H, broad), 7.72 (1H, t, J=8.5 Hz), 7.72-7.91 (1H, two broad peaks, Z/E forms), 8.49 (1H, d, J=5.0 Hz), 9.22-9.35 (1H, two broad peaks. Z/E forms). MW 362.48. LCMS t_{R} (min): 1.74. MS (APCI), m/z 363.23 [M+H]⁺. HPLC t_{R} (min): 10.41. M_{P} 102.6-103.6° C.

### 5. N-(3-Isopropyl-phenyl)-N′-(1-methanesulfonyl-piperidin-4-yl)-6-propyl-[1,3,5]triazine-2,4-diamine

A mixture of (4-Chloro-6-propyl-[1,3,5]triazin-2-yl)-(3-isopropyl-phenyl)-amine (0.2 g, 0.69 mmol), 1-methanesulfonyl-piperidin-4-ylamine hydrochloride (0.15 g, 0.7 mmol) and powdered K₂CO₃ (0.191 g, 1.38 mmol) in DMSO (1 mL) was stirred for 1.5 hour at 80° C. When the reaction was over according to TLC, the mixture was cooled and diluted with water. The formed precipitate was filtered off, washed with water, dried and purified by column chromatography on silica gel (EtOAc/hexane, 1/1) that gave the final compound. Yield 250 mg, 84%. ¹H-NMR (400 MHz, DMSO-D₆) δ_{H}: 0.92 (3H, t, J=7.5 Hz), 1.20 (6H, d, J=7.5 Hz), 1.59 (2H, m), 1.73 (2H, m), 1.95 (2H, m), 2.40 (2H, broad t, J=7.5 Hz), 2.85 (6H, superposition of s and two m), 3.55 (2H, m), 3.95 (1H, broad peak, Z/E forms), 6.85 (1H, d, J=8.5 Hz), 7.18 (1H, broad t, J=8.5 Hz), 7.50 (2H, broad peak, Z/E forms), 7.72 (1H, broad peak), 9.12-9.30 (1H, two broad peaks, Z/E forms). MW 432.59. LCMS t_{R} (min): 1.84. MS (APCI), m/z 433.22 [M+H]⁺. HPLC t_{R} (min): 11.98. M_{P} 80.4-81.8° C.

### 6. N-(1H-Indazol-6-yl)-N′-(1-methanesulfonyl-piperidin-4-yl)-6-propyl-[1,3,5]-triazine-2,4-diamine

To a solution of 2,4-dichloro-6-propyl-[1,3,5]triazine (1.5 g, 7.81 mmol) in THF (10 mL) a solution of 1H-indazol-6-ylamine (1.04 g, 7.81 mmol), DIPEA (1.0 g 7.81 mmol) in THF (10 mL) was added slowly dropwise at 0° C. The resulting mixture was stirred at 0° C. for 3 hours (TLC control), warmed up to RT and stirred at RT for 1 hour, diluted with water and ether. The organic phase was dried over Na₂SO₄ and concentrated. Purification by column chromatography on silica gel (30% acetone/DCM) gave (4-Chloro-6-propyl-[1,3,5]triazin-2-yl)-(1H-indazol-6-yl)-amine. Yield 2 g, 88%.

A mixture of (4-Chloro-6-propyl-[1,3,5]triazin-2-yl)-(1H-indazol-6-yl)-amine (0.231 g, 0.8 mmol), 1-methanesulfonyl-piperidin-4-ylamine hydrochloride (0.172 g, 0.8 mmol) and triethylamine (0.162 g, 1.6 mmol) in acetonitrile (10 mL) was refluxed for 4 hours. When the reaction was over according to TLC, the mixture was cooled, and filtered. The filtrated was concentrated in vacuum, washed with water, dried and purified by column chromatography on silica gel (ethyl acetate/methanol, 50/1) that gave the final compound. Yield 80 mg, 23%. ¹H-NMR (400 MHz, DMSO-D₆) δ_{H}: 0.94 (3H, t, J=7.5 Hz), 1.60 (2H, m), 1.73 (2H, q, J=7.5 Hz), 2.00 (2H, broad), 2.45 (2H, m), 2.90 (3H, broad peak), 2.96 (2H, broad peak), 3.57 (2H, m), 3.99 (1H, broad peak), 7.22-7.35 (1H, broad peak, Z/E forms), 7.43-7.51 (1H, broad peak, Z/E forms), 7.60 (1H, d, J=8.5 Hz), 7.91 (1H, s), 8.00-8.18 (1H, broad peak, Z/E forms), 9.39-9.55 (1H, broad peak, Z/E forms), 12.67-12.80 (1H, broad peak, Z/E forms).

MW 430.55. LCMS t_{R} (min): 1.46. MS (APCI), m/z 431.16 [M+H]⁺. HPLC t_{R} (min): 8.99. M_{P} 219.3-221.0° C.

### 7. N-(2,3-Dihydro-1H-indol-6-yl)-N′-(4-fluoro-benzyl)-6-propyl-[1,3,5]-triazine-2,4-diamine

To a solution of 2,4-dichloro-6-propyl-[1,3,5]triazine (400 mg, 2.08 mmol) and DIPEA (296 mg, 2.29 mmol) in THF (5 mL) a solution of (4-fluoro-benzyl)-amine (261 mg, 2.08 mmol) in THF (5 mL) was added slowly dropwise at 0° C. The resulting mixture was stirred at 0° C. for 1 hour (TLC control), diluted with water, and extracted with ethyl acetate. The organic layers were combined and stirred with Na₂SO₄, and concentrated. The residue was triturated with hexane. The formed precipitate was filtered, washed with hexane and dried on air. As a result, (4-Chloro-6-propyl-[1,3,5]-triazin-2-yl)-(4-fluoro-benzyl)-amine was obtained as yellow crystals. Yield 420 mg, 72%.

To a solution of (4-Chloro-6-propyl-[1,3,5]triazin-2-yl)-(4-fluoro-benzyl)-amine (415 mg, 1.48 mmol) and 6-amino-2,3-dihydro-indole-1-carboxylic acid tert-butyl ester (381 mg, 1.63 mmol) in DMSO (4 mL) K₂CO₃ (408 mg, 2.96 mmol) was added. The mixture was stirred for 3 hours at 150° C. When the reaction was over according to TLC, the mixture was cooled down to room temperature, and diluted with water (50 mL). The formed precipitate was filtered off, and dried that gave 6-[4-(4-Fluoro-benzylamino)-6-propyl-[1,3,5]triazin-2-ylamino]-2,3-dihydro-indole-1-carboxylic acid tert-butyl ester. Yield 560 mg, 79%.

The BOC-intermediate (560 mg, 1.17 mmol) was dissolved in iso-propyl alcohol (25 ml), and 5-6N HCl in i-PrOH (10 mL) was added. The mixture was stirred at 50° C. for 4 hours. The solvent and excess of HCl were evaporated. The obtained residue was neutralized with 30% aqueous solution of NaOH, and the product was extracted with chloroform. The extract was concentrated, and the resulting residue was purified by column chromatography on silica gel (ethyl acetate/hexane, 1/2.5). After the additional column chromatography on silica gel (acetonitrile/methanol) the final compound 33 obtained with purity of 94.18% (HPLC). Yield 100 mg, 23%. ¹H-NMR (400 MHz, DMSO-D₆) δ_{H}: 0.90 (3H, broad), 1.70 (2H, broad), 2.39 (2H, broad), 2.82 (2H, t, J=7.5 Hz), 3.39 (2H, t, J=7.5 Hz), 4.48 (2H, d, J=7.5 Hz), 5.39 (1H, broad peak. Z/E forms), 6.85 (1H, broad peak), 6.92 (1H, broad peak, Z/E forms), 6.97-7.06 (1H, broad, Z/E forms), 7.11 (2H, d/d, J=8.5/8.0 Hz), 7.35 (2H, broad m), 7.67-7.88 (1H, broad peak. Z/E forms), 9.00-9.13 (1H, broad peak. Z/E forms). MW 378.46. LCMS t_{R} (min): 1.52. MS (APCI), m/z 379.19 [M+H]⁺. HPLC t_{R} (min): 8.90. M_{P} 122-124° C.

### 8. N-4-Fluoro-benzyl)-N′-(1H-indazol-6-yl)-6-propyl-[1,3,5]triazine-2,4-diamine

A mixture of 2-(1H-indazol-6-yl)-4-chloro-6-propyl-[1,3,5]triazine-amine (0.289 g, 1.0 mmol), 4-fluoro-benzylamine (0.125 g, 1.0 mmol) and powdered K₂CO₃ (0.276 g, 2.0 mmol) in DMSO (1 mL) was stirred for 1.5 hours at 80° C. When the reaction was over according to TLC, the mixture was cooled down to room temperature and diluted with water. The formed precipitate was filtered off, washed with water, dried and dissolved in dichloromethane. The unresolved solids were filtered off, and filtrate was purified by column chromatography on silica gel (EtOAc/hexane) that gave the final compound. Yield 60 mg, 16%. ¹H-NMR (400 MHz, DMSO-D₆) δ_{H}: 0.93 (3H, broad), 1.73 (2H, broad), 2.45 (2H, broad), 4.52 (2H, broad peak), 7.10 (2H, broad t, J=8.5/8.0 Hz, Z/E forms), 7.38 (3H, broad peak), 7.56 (1H, d, J=8.5 Hz), 7.72-7.97 (1H, broad peak. Z/E forms), 7.90 (1H, s), 8.04-8.13 (1H, broad peak. Z/E forms), 9.40-9.58 (1H, broad peak. Z/E forms), 12.73 (1H, broad peak, Z/E forms).

MW 377.43. LCMS t_{R} (min): 1.64. MS (APCI), m/z 378.17 [M+H]⁺. HPLC t_{R} (min): 10.65. M_{P} 185-187° C.

### 9. N-(3-Dimethylamino-phenyl)-N′-(1-methanesulfonyl-piperidin-4-yl)-6-propyl-[1,3,5]triazine-2,4-diamine

The mixture of 2,4-dichloro-6-propyl-[1,3,5]triazine (210 mg, 1.09 mmol), N,N-dimethyl-benzene-1,3-diamine dihydrochloride (229 mg, 1.09 mmol) and DIPEA (426 mg, 3.30 mmol) dissolved in acetonitrile (5 mL) was stirred at room temperature for 3 hours. Then, the mixture was diluted with water and extracted with DCM. The organic phase was concentrated. The residue was purified by column chromatography on silica gel (2% acetone/DCM) giving the compound N-(4-Chloro-6-propyl-[1,3,5]triazin-2-yl)-N′,N′-dimethyl-benzene-1,3-diamine. Yield 120 mg, 38%.

A mixture of N-(4-Chloro-6-propyl-[1,3,5]-triazin-2-yl)-N′,N′-dimethyl-benzene-1,3-diamine (120 mg, 0.41 mmol), 1-methanesulfonyl-piperidin-4-ylamine hydrochloride (108 mg, 0.50 mmol) and DIPEA (155 mg, 1.20 mmol) dissolved in acetonitrile (4 mL) was stirred at refluxing for 12 hours. The reaction was monitored by TLC (DCM/ethyl acetate, 5/1). When the reaction was over, a target compound was extracted with DCM. The organic phases were combined, dried over Na₂SO₄, and concentrated. The residue was purified by column chromatography on silica gel (DCM/MeOH, 10/1). The fractions containing the product were concentrated, and the resulting foam was triturated with c-hexane that gave the final compound as white powder. Yield 149 mg, 79%. ¹H-NMR (400 MHz, DMSO-D₆) δ_{H}: 1.94 (4H, broad), 3.68 (4H, broad peak), 5.32 (2H, s), 7.27 (5H, m), 7.37 (1H, d/d, J=8.5/8.0 Hz), 7.72 (1H, broad peak), 8.02 (1H, s), 8.40 (1H, broad peak), 9.72 (1H, broad peak). MW 433.58. LCMS t_{R} (min): 1.54. MS (APCI), m/z 434.21 [M+H]⁺. HPLC t_{R} (min): 8.43. Mp 134-136° C.

### 10. N-(3-Chloro-4-fluoro-phenyl)-N′-(1-methanesulfonyl-piperidin-4-yl)-6-(3,3,3-trifluoro-propyl)-[1,3,5]triazine-2,4-diamine

To solution of cyanuric chloride in DCM (20 mL) a 6.44M solution of n-3,3,3-trifluoro-propyl magnesium bromide in THF (15 mL) was added at −15° C. The resulting mixture was stirred at −15° C. for 3.5 hours, poured to water, extracted with DCM. The combined organic layers was washed with water, dried over Na₂SO₄ and evaporated. Purification by column chromatography on silica gel (DCM) gave 2,4-Dichloro-6-(3,3,3-trifluoro-propyl)-[1,3,5]triazine as oil. Yield 679 mg, 43%.

To a solution of 2,4-Dichloro-6-(3,3,3-trifluoro-propyl)-[1,3,5]-triazine (679 g, 2.76 mmol) in THF (8 mL) a solution of 3-chloro-4-fluoro-phenylamine (402 mg, 2.76 mmol) and NEt₃ (279 mg 2.76 mmol) in THF (5 mL) was added slowly dropwise at 0° C. The resulting mixture was stirred at 0° C. for 40 minutes, warmed up to room temperature and stirred at room temperature for 8 hours, diluted with water and ethyl acetate. The organic phases was dried over Na₂SO₄ and concentrated. Purification by column chromatography on silica gel (hexane/DCM) gave compound (3-Chloro-4-fluoro-phenyl)-[4-chloro-6-(3,3,3-trifluoro-propyl)-[1,3,5]triazin-2-yl]-amine. Yield 580 mg, 59%.

A mixture of (3-Chloro-4-fluoro-phenyl)-[4-chloro-6-(3,3,3-trifluoro-propyl)-[1,3,5]triazin-2-yl]-amine (250 mg, 0.7 mmol), 1-methanesulfonyl-piperidin-4-ylamine hydrochloride (2) (172 mg, 0.80 mmol) and powdered K₂CO₃ (304 mg, 2.20 mmol) in DMSO (4 mL) was stirred for 6.5 hours at 50° C. When the reaction was over according to TLC, the mixture was cooled down to room temperature, diluted with water. The residue was filtered and washed with water. Purification by column chromatography on silica gel (ethyl acetate/hexane) gave a final compound. Yield 76 mg, 22%. ¹H-NMR (400 MHz, DMSO-D₆) δ_{H}: 1.61 (2H, m), 2.05 (2H, m), 2.69 (4H, m), 2.91 (5H, superposition of s (3H) and m (2H)), 3.61 (2H, m), 3.91-3.98 (1H, two broad peaks. Z/E forms), 7.31 (1H, superposition of m. Z/E forms), 7.51-7.61 (1H, two broad peaks. Z/E forms), 7.71 (1H, broad peak, Z/E forms), 8.05-8.19 (1H, two broad peaks, Z/E forms), 9.51-9.71 (1H, two broad peaks. Z/E forms). MW 496.91. LCMS t_{R} (min): 1.90. MS (APCI+), m/z 497.10, 499.09 [M+H]⁺. HPLC t_{R} (min): 13.94. M_{P} 192-193° C.

### 11. 4-(2-phenylethyl)-6-(2,2,2-trifluoroethoxy)-N-[3-(trifluoromethyl)phenyl]-1,3,5-triazin-2-amine

LCMS: M+1=443; ¹H NMR, CDCl₃, ppm: 3.22 m (4H, CH); 4.6 m (4H CH); 7.22 m (6H, CH); 7.4 d (1H, CH); 7.5 t (1H, CH); 7.66 s (1H; CH;); 8.2 s (1H, NH).

### Section 3. Preparation of R4-Triazine Libraries

Several synthetic schemes were applied to synthesize ‘R4-variation’ subset.

### Preparation of O,N,N-Triazines in Tables 9, 10, 11 and 12

It is facile to scale up a key intermediate 1 from a two steps synthesis from 2,4,6-trichlor-1,3,5-triazine by reaction with amine at −20° C. followed by the reaction with sodium and alcohol. Several activated and deactivated aromatic amines were used for the preparation of the desired compounds. Depending on aniline reactivity, the reaction conditions with variation of time, solvent, base and temperature were optimized for each single compound under reaction conditions listed in the following table. In general, R6-R2-R4 route was used to prepare this library (Scheme 9). In some exceptional cases, a general synthetic route gave very low yields and therefore elaboration of alternative approaches (Schemes 10, 11 and 12) using some other order of introduction of fragments was employed and described in the experimental procedures.

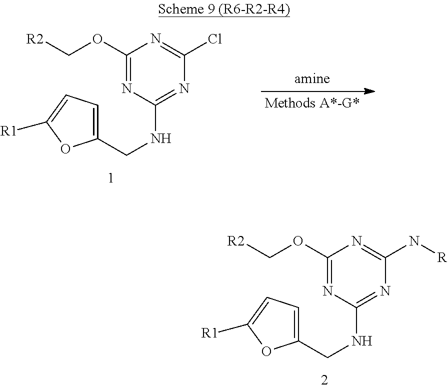

| Method | Conditions |
|---|---|
| A* | DMF, K₂CO₃, 100-150^{° C.}, 2-4 h |
| B* | DMSO, K₂CO₃, 80-150^{° C.}, 2-4 h |
| C* | organic base, fusion, 1-8 h |
| D* | Pd₂dba₃, t-Bu₃P, t-BuONa, toluene, 130^{° C.}, 1 h |
| E* | acetone, KOH, refluxing, 20 h |
| F* | MW irradiation, organic base, acetinitrile, 140^{° C.}, |
| | 5-60 min |
| G* | DIPEA, dioxane, 70^{° C.}, 8 h |
| H* | DIPEA, acetonitrile, RT, 4 h |
| I* | NEt₃, acetonitrile/DMSO, 100^{° C.}, 14 h |
| J* | NaOAc, acetic acid, 50^{° C.}, 3 h |
| K* | NEt₃ or DIPEA, acetonitrile, refluxing, 3 h |
| L* | NaHCO₃, Et, refluxing, 1 h |
| M* | NaHCO₃, acetonitrile, 60^{° C.}, 2 h |
| N* | DIPEA, THF, 50^{° C.}, 5 h |
| O* | Pd(OAc)₂, t-Bu₃P, K₂CO₃, toluene, refluxing, 3 h |

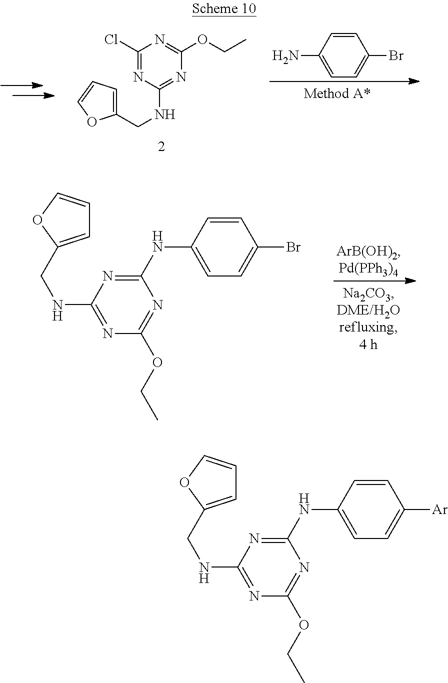

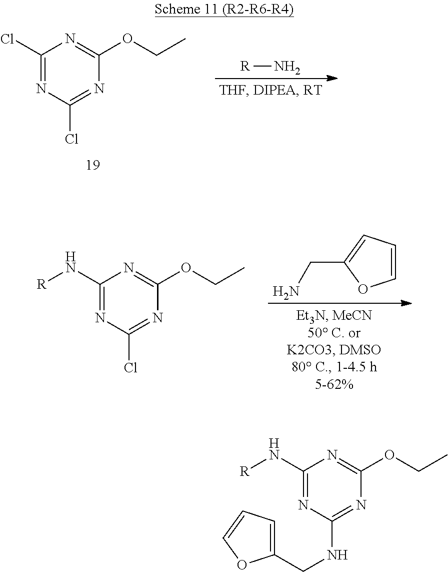

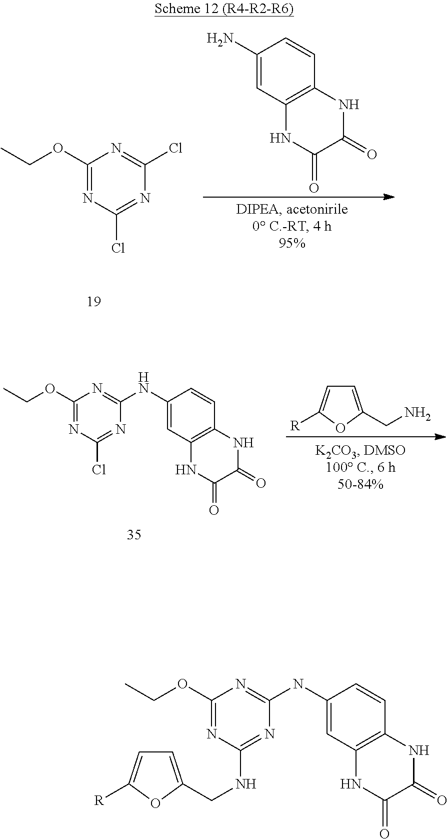

TABLE 9

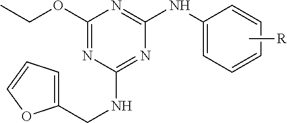

| En | Structure | IUPAC Name | MW | Formula |
|---|---|---|---|---|
| 1 | 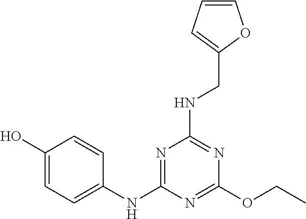 | 4-({4-ethoxy-6-[(furan-2- ylmethyl)amino]-1,3,5- triazin-2-yl}amino) phenol | 327.3 | C₁₇H₁₈N₆O₃ |
| 2 | 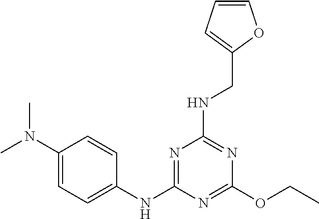 | N-[4-(dimethylamino) phenyl]-6-ethoxy-N′- (furan-2-ylmethyl)-1,3,5- triazine-2,4-diamine | 354.4 | C₁₈H₁₈N₆O₂S |
| 3 | 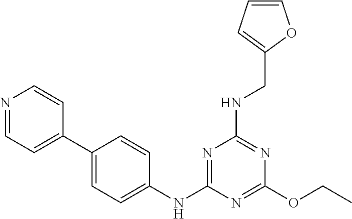 | 6-ethoxy-N-(furan-2- ylmethyl)-N′-[4- (pyridin-4-yl)phenyl]- 1,3,5-triazine- 2,4-diamine | 388.4 | C₂₂H₁₉N₇O₃ |
| 4 | 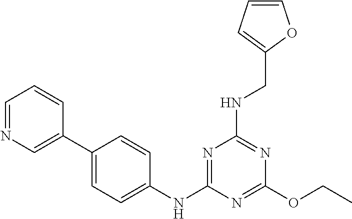 | 6-ethoxy-N-(furan-2- ylmethyl)-N′-[4- (pyridin-3-yl)phenyl]- 1,3,5-triazine- 2,4-diamine | 388.4 | C₂₁H₂₀N₆O₂ |
| 5 | 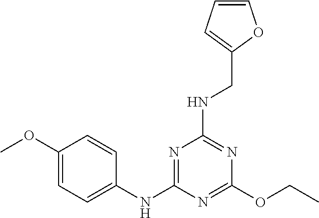 | 6-ethoxy-N-(furan-2- ylmethyl)-N′-(4- methoxyphenyl)-1,3,5- triazine-2,4-diamine | 341.4 | C₁₇H₁₉N₅O₃ |
| 6 | 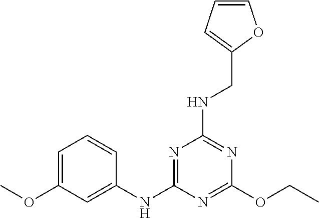 | 6-ethoxy-N-(furan-2- ylmethyl)-N′-(3- methoxyphenyl)-1,3,5- triazine-2,4-diamine | 341.4 | C₁₇H₁₆F₃N₅O₃ |
| 7 | 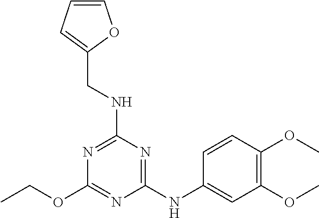 | N-(3,4-dimethoxyphenyl)- 6-ethoxy-N′-furan-2- ylmethyl)-1,3,5-triazine- 2,4-diamine | 371.4 | C₁₆H₁₆ClN₅O₂ |
| 8 | 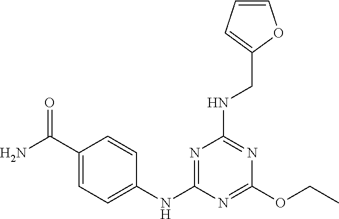 | 4-({4-ethoxy-6- [(furan-2- ylmethyl)amino]- 1,3,5-triazin-2- yl}amino)benzamide | 354.4 | C₁₈H₂₂N₆O₂ |
| 9 | 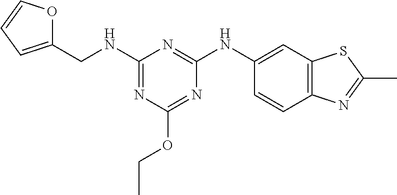 | 6-ethoxy-N-(furan-2- ylmethyl)-N′-(2- methyl-1,3-benzothiazol- 6-yl)-1,3,5- triazine-2,4-diamine | 382.4 | C₁₇H₁₉N₅O₂ |
| 10 | 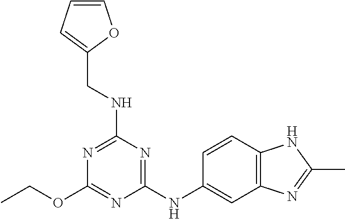 | 6-ethoxy-N-(furan-2- ylmethyl)-N′-(2- methyl-1H-benzimidazol- 6-yl)-1,3,5- triazine-2,4-diamine | 365.4 | C₁₇H₁₉N₅O₃ |
| 11 | 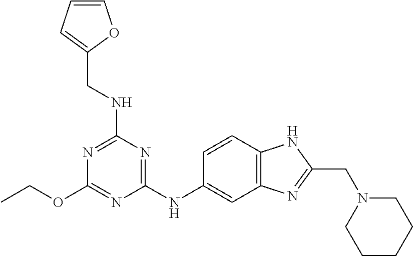 | 6-ethoxy-N-(furan-2- ylmethyl)-N′-[2- (piperidin-1- ylmethyl)-1H- benzimidazol-6-yl]- 1,3,5-triazine- 2,4-diamine | 448.5 | C₂₂H₂₆N₆O₃ |
| 12 | 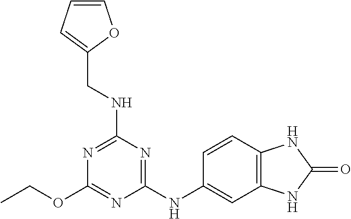 | 5-({4-ethoxy- 6-[(furan-2- ylmethyl)amino]- 1,3,5-triazin-2- yl}amino)-1,3- dihydro-2H- benzimidazol-2-one | 367.4 | C₁₇H₁₅F₃N₄O₃ |
| 13 | 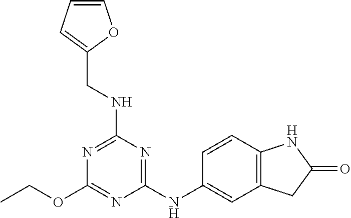 | 5-({4-ethoxy-6- [(furan-2- ylmethyl)amino]- 1,3,5-triazin-2- yl}amino)-1,3-dihydro- 2H-indol-2-one | 366.4 | C₂₃H₂₈N₈O₂ |
| 14 | 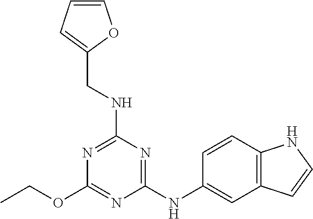 | 6-ethoxy-N-(furan-2- ylmethyl)-N′- (1H-indol-5-yl)-1,3,5- triazine-2,4- diamine | 350.4 | C₁₇H₁₇N₇O₃ |
| 15 | 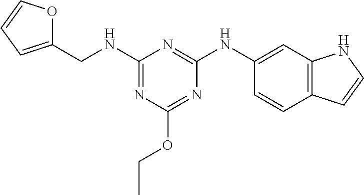 | 6-ethoxy-N-(furan-2- ylmethyl)-N′- (1H-indol-6-yl)- 1,3,5-triazine-2,4- diamine | 350.4 | C₁₈H₁₈N₆O₂ |
| 16 | 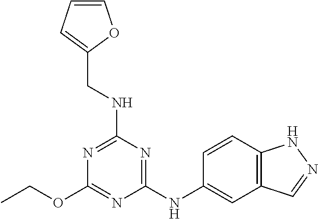 | 6-ethoxy-N-(furan-2- ylmethyl)-N′- (1H-indazol-5-yl)- 1,3,5-triazine-2,4- diamine | 351.4 | C₁₈H₁₉N₇O₂ |
| 17 | 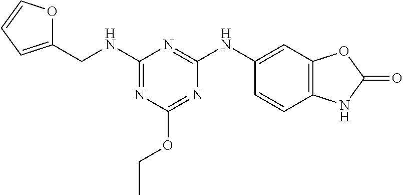 | 6-({4-ethoxy- 6-[(furan-2- ylmethyl)amino]- 1,3,5-triazin-2- yl}amino)-1,3- benzoxazol-2(3H)- one | 368.3 | C₁₈H₁₇N₇O₄ |
| 18 | 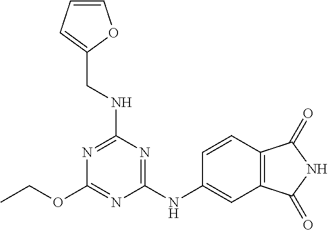 | 5-({4-ethoxy-6- [(furan-2- ylmethyl)amino]- 1,3,5-triazin-2- yl}amino)-1H- isoindole-1,3(2H)- dione | 380.4 | C₁₉H₂₃N₅O₃ |
| 19 | 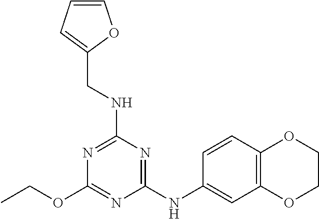 | N-(2,3-dihydro-1,4- benzodioxin-6- yl)-6-ethoxy-N′- (furan-2-ylmethyl)- 1,3,5-triazine-2,4-diamine | 369.4 | C₁₈H₁₈N₆O₂ |
| 20 | 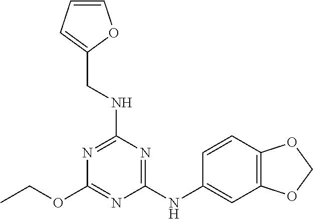 | N-(1,3-benzodioxol- 5-yl)-6-ethoxy- N′-(furan-2-ylmethyl)- 1,3,5-triazine- 2,4-diamine | 355.3 | C₂₀H₂₄N₆O₂ |
| 21 | 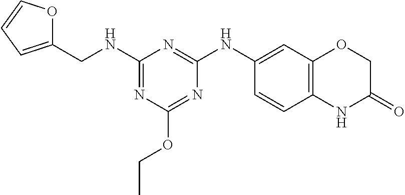 | 7-({4-ethoxy-6- [(furan-2- ylmethyl)amino]- 1,3,5-triazin-2- yl}amino)-2H- 1,4-benzoxazin- 3(4H)-one | 382.4 | C₁₆H₁₇N₅O₃ |
| 22 | 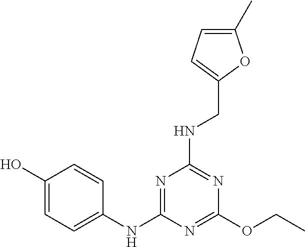 | 4-[(4-ethoxy-6- {[(5-methylfuran-2- yl)methyl]amino}- 1,3,5-triazin-2- yl)amino]phenol | 341.4 | C₁₆H₁₅ClN₄O₃ |
| 23 | 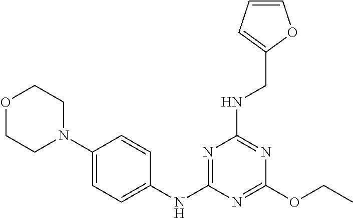 | 6-ethoxy-N-(furan-2- ylmethyl)-N′-[4- (morpholin-4- yl)phenyl]-1,3,5- triazine-2,4-diamine | 396.4 | C₁₈H₁₈N₆O₄ |
| 24 | 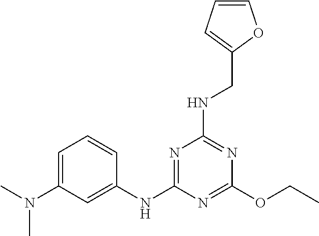 | N-[3-(dimethylamino) phenyl]-6- ethoxy-N′-(furan-2- ylmethyl)-1,3,5- triazine-2,4-diamine | 354.4 | C₂₂H₂₆N₈O₃ |
| 25 | 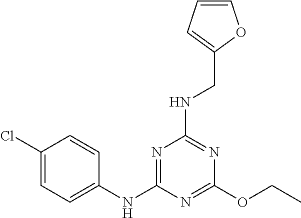 | N-(4-chlorophenyl)- 6-ethoxy-N′- (furan-2-ylmethyl)- 1,3,5-triazine- 2,4-diamine | 345.8 | C₂₀H₂₄N₆O₃ |
| 26 | 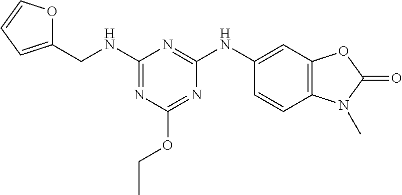 | 6-({4-ethoxy-6- [(furan-2- ylmethyl)amino]- 1,3,5-triazin-2- yl}amino)-3-methyl- 1,3-benzoxazol- 2(3H)-one | 382.4 | C₂₂H₂₁N₅O₃ |
| 27 | 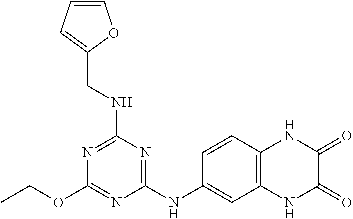 | 6-({4-ethoxy-6- [(furan-2- ylmethyl)amino]- 1,3,5-triazin-2- yl}amino)-1,4- dihydroquinoxaline- 2,3-dione | 395.4 | C₁₉H₁₉N₇O₂ |
| 28 | 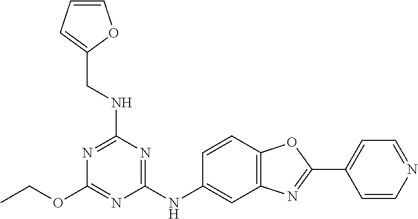 | 6-ethoxy-N-(furan-2- ylmethyl)-N′-[2- (pyridin-4-yl)-1,3- benzoxazol-5-yl]- 1,3,5-triazine-2,4-diamine | 429.4 | C₁₈H₁₈N₆O₄ |
| 29 | 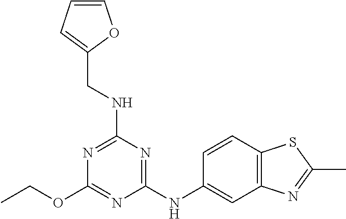 | 6-ethoxy-N-(furan-2- ylmethyl)-N′-(2- methyl-1,3-benzothiazol- 5-yl)-1,3,5- triazine-2,4-diamine | 382.4 | C₁₇H₁₇N₇O₂ |
| 30 | 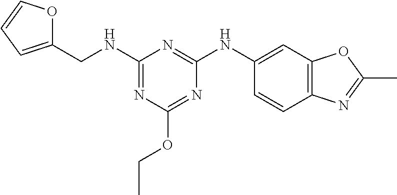 | 6-ethoxy-N-(furan-2- ylmethyl)-N′-(2- methyl-1,3-benzoxazol- 6-yl)-1,3,5- triazine-2,4-diamine | 366.4 | C₁₈H₂₂N₆O₂ |
| 31 | 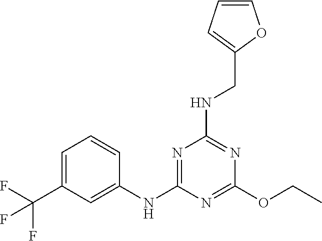 | 6-ethoxy-N-(furan-2- ylmethyl)-N′-[3- (trifluoromethyl) phenyl]-1,3.5- triazine-2,4-diamine | 379.3 | C₂₂H₂₈N₆O₂ |
| 32 | 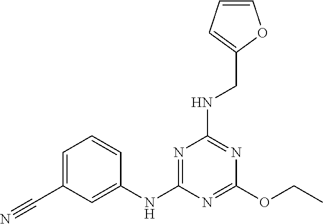 | 3-({4-ethoxy-6- [(furan-2- ylmethyl)amino]- 1,3,5-triazin-2- yl}amino)benzonitrile | 336.3 | C₂₁H₂₀N₆O₂ |
| 33 | 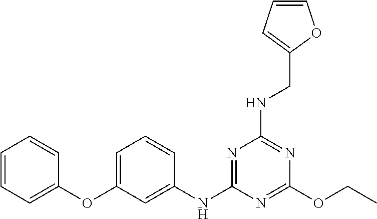 | 6-ethoxy-N-(furan-2- ylmethyl)-N′-(3- phenoxyphenyl)-1,3,5- triazine-2,4- diamine | 403.4 | C₁₇H₁₆F₃N₅O₂ |
| 34 | 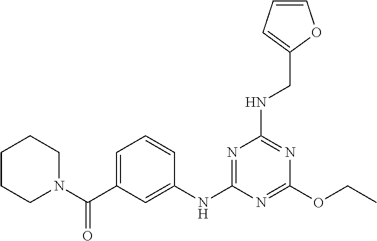 | [3-({4-ethoxy- 6-[(furan-2- ylmethyl)amino]- 1,3,5-triazin-2- yl}amino)phenyl] (piperidin-1- yl)methanone | 422.5 | C₁₇H₁₇N₇O₂ |
| 35 | 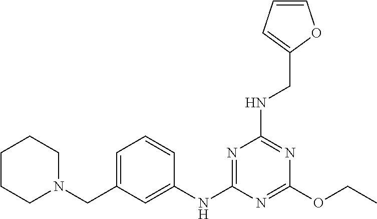 | 6-ethoxy-N-(furan-2- ylmethyl)-N′-[3- (piperidin-1-ylmethyl) phenyl]-1,3,5- triazine-2,4-diamine | 408.5 | C₁₈H₁₈N₆O₃ |
| 36 | 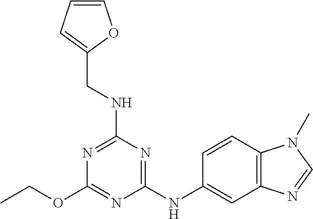 | 6-ethoxy-N-(furan-2- ylmethyl)-N′-(1- methyl-1H-benzimidazol- 5-yl)-1,3,5- triazine-2,4-diamine | 365.4 | C₂₃H₂₈N₈O₃ |
| 37 | 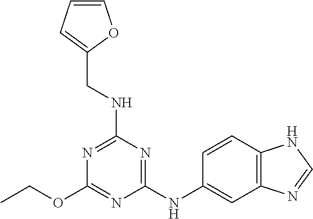 | N-(1H-benzimidazol-5- yl)-6-ethoxy- N′-furan-2-ylmethyl)- 1,3,5-triazine- 2,4-diamine | 351.4 | C₂₁H₁₉N₇O₂S |
| 38 | 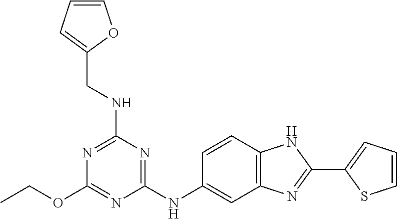 | 6-ethoxy-N-(furan-2- ylmethyl)-N′-[2- (thiophen-2-yl)- 1H-benzimidazol-5- yl]-1,3,5-triazine- 2,4-diamine | 433.5 | C₁₈H₁₆F₃N₇O₂ |
| 39 | 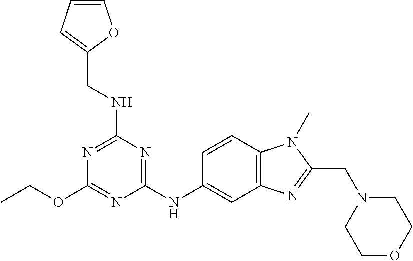 | 6-ethoxy-N-(furan-2- ylmethyl)-N′-[1- methyl-2-(morpholin- 4-ylmethyl)- 1H-benzimidazol- 5-yl]-1,3,5- triazine-2,4-diamine | 464.5 | C₁₆H₁₆BrN₅O₂ |
| 40 | 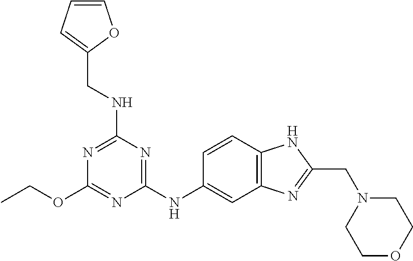 | 6-ethoxy-N-(furan-2- ylmethyl)-N′-[2- (morpholin-4- ylmethyl)-1H- benzimidazol-5-yl]- 1,3,5-triazine- 2,4-diamine | 450.5 | C₂₅H₃₄N₆O₄ |
| 41 | 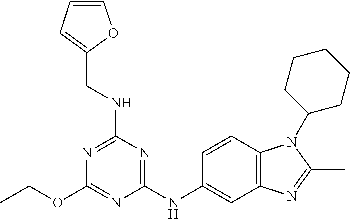 | N-(1-cyclohexyl- 2-methyl-1H- benzimidazol-5-yl)- 6-ethoxy-N′- (furan-2-ylmethyl)- 1,3,5-triazine- 2,4-diamine | 447.5 | C₁₈H₁₉N₇O₂ |
| 42 | 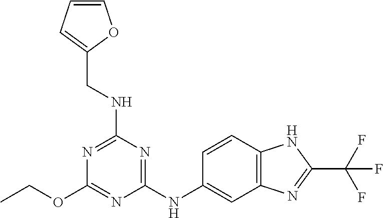 | 6-ethoxy-N-(furan-2- ylmethyl)-N′-[2- (trifluoromethyl)-1H- benzimidazol-5- yl]-1,3,5-triazine- 2,4-diamine | 419.4 | C₂₄H₂₉N₇O₂ |
| 43 | 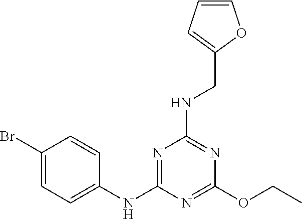 | N-(4-bromophenyl)- 6-ethoxy-N′- (furan-2-ylmethyl)- 1,3,5-triazine- 2,4-diamine | 390.2 | C₁₇H₁₆N₆O₂ |
| 44 | 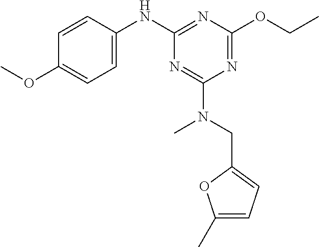 | 6-ethoxy-N′-(4- methoxyphenyl)-N- methyl-N-[(5- methylfuran-2- yl)methyl]-1,3,5- triazine-2,4-diamine | 369.4 | C₂₂H₂₀N₈O₂ |
| 45 | 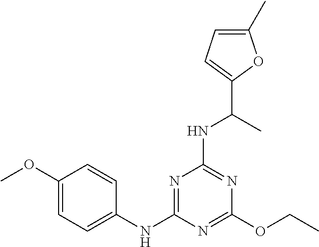 | 6-ethoxy-N-(4- methoxyphenyl)-N′- [1-(5-methylfuran- 2-yl)ethyl]-1,3,5- triazine-2,4-diamine | 369.4 | C₂₀H₂₂N₆O₃ |
| 46 | 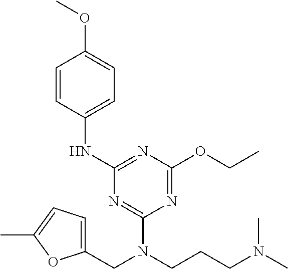 | N-[3-(dimethylamino) propyl]-6- ethoxy-N′-(4- methoxyphenyl)-N-[(5- methylfuran-2- yl)methyl]-1,3,5- triazine-2,4-diamine | 440.5 | C₂₀H₂₄N₆O₂ |
| 47 | 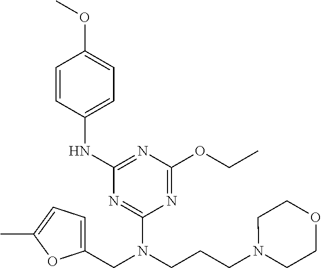 | 6-ethoxy-N′-(4- methoxyphenyl)-N- [(5-methylfuran-2- yl)methyl]-N-[3- (morpholin-4- yl)propyl]-1,3,5- triazine-2,4-diamine | 482.6 | C₂₃H₃₂N₆O₃ |
| 48 | 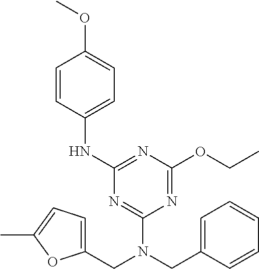 | N-benzyl-6-ethoxy-N′-(4- methoxyphenyl)-N- [(5-methylfuran- 2-yl)methyl]- 1,3,5-triazine-2,4- diamine | 445.5 | C₁₉H₂₃N₅O₃ |
| 49 | 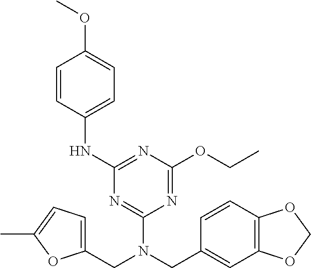 | N-(1,3-benzodioxol- 5-ylmethyl)-6- ethoxy-N′-(4- methoxyphenyl)-N-[(5- methylfuran-2- yl)methyl]-1,3,5- triazine-2,4-diamine | 489.5 | C₂₃H₂₇N₇O₂ |
| 50 | 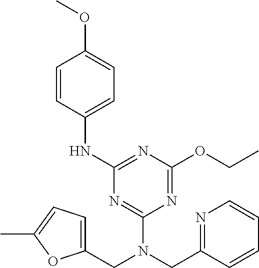 | 6-ethoxy-N′-(4- methoxyphenyl)-N- [(5-methylfuran-2- yl)methyl]-N- (pyridin-2-ylmethyl)- 1,3,5-triazine- 2,4-diamine | 446.5 | C₂₄H₂₇F₃N₈O₅ |
| 51 | 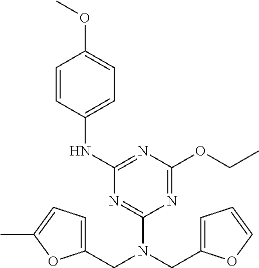 | 6-ethoxy-N-(furan-2- ylmethyl)-N′-(4- methoxyphenyl)-N- [(5-methylfuran- 2-yl)methyl]-1,3,5- triazine-2,4- diamine | 435.5 | C₂₆H₂₇N₅O₅ |
| 52 | 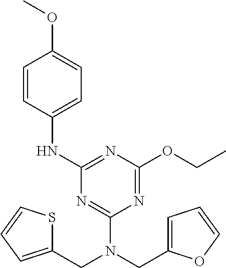 | 6-ethoxy-N-(furan-2- ylmethyl)-N′-(4- methoxyphenyl)- N-(thiophen-2- ylmethyl)-1,3,5- triazine-2,4-diamine | 437.5 | C₂₄H₂₆N₆O₃ |
| 53 | 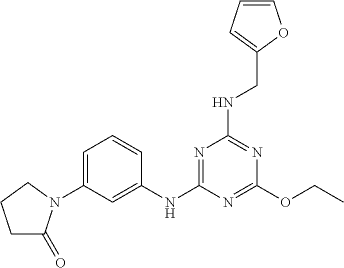 | 1-[3-({4-ethoxy-6- [(furan-2- ylmethyl)amino]- 1,3,5-triazin-2- yl}amino)phenyl] pyrrolidin-2-one | 394.4 | C₁₇H₁₉N₅O₃ |
| 54 | 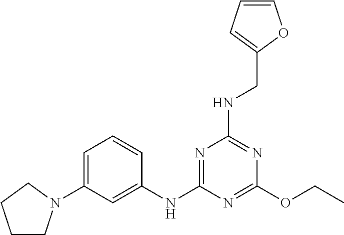 | 6-ethoxy-N-(furan-2- ylmethyl)-N′-[3- (pyrrolidin-1-yl) phenyl]-1,3,5- triazine-2,4-diamine | 380.4 | C₁₈H₂₂N₆O₂ |
| 55 | 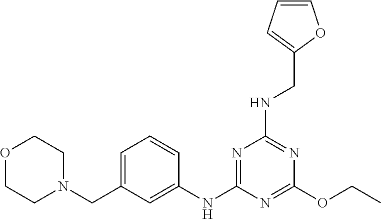 | 6-ethoxy-N-(furan-2- ylmethyl)-N′-[3- (morpholin-4- ylmethyl)phenyl]- 1,3,5-triazine-2,4-diamine | 410.5 | C₁₈H₁₈N₆O₂S |
| 56 | 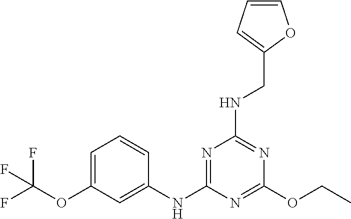 | 6-ethoxy-N-(furan-2- ylmethyl)-N′-[3- (trifluoromethoxy) phenyl]-1,3,5- triazine-2,4-diamine | 395.3 | C₂₁H₂₆N₆O₃ |
| 57 | 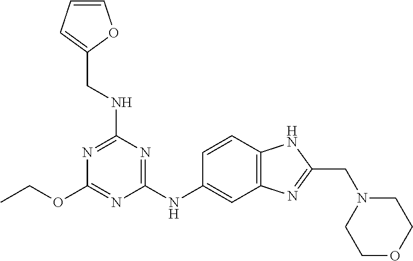 | 6-ethoxy-N-(furan-2- ylmethyl)-N′-[2- (morpholin-4- ylmethyl)-1H- benzimidazol-5-yl]- 1,3,5-triazine- 2,4-diamine trifluoroacetate | 450.5 | C₁₈H₁₆N₆O₄ |
| 58 | 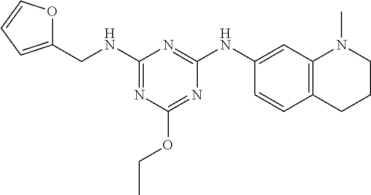 | 6-ethoxy-N-(furan-2- ylmethyl)-N′-(1- methyl-1,2,3,4- tetrahydroquinolin-7- yl)-1,3,5-triazine- 2,4-diamine | 380.4 | C₁₈H₁₈N₈O₂ |
| 59 | 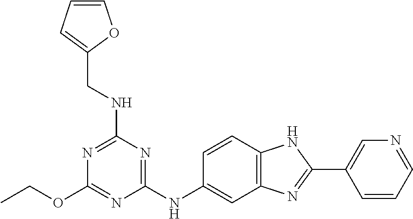 | 6-ethoxy-N-(furan-2- ylmethyl)-N′-[2- (pyridin-3-yl)-1H- benzimidazol-5-yl]- 1,3,5-triazine-2,4-diamine | 428.4 | C₂₃H₂₅N₅O₄ |
| 60 | 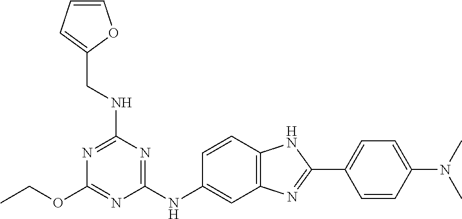 | N-{2-[4-(dimethylamino) phenyl]-1H- benzimidazol-5-yl}- 6-ethoxy-N′- (furan-2-ylmethyl)- 1,3,5-triazine- 2,4-diamine | 470.5 | C₁₇H₁₆N₆O₂S |
| 61 | 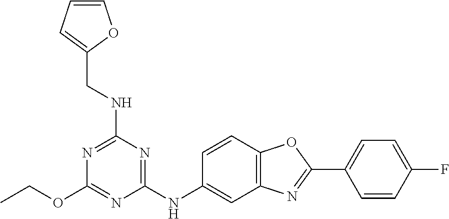 | 6-ethoxy-N-[2-(4- fluorophenyl)-1,3- benzoxazol-5-yl]- N′-(furan-2- ylmethyl)-1,3,5- triazine-2,4-diamine | 446.4 | C₂₂H₂₇N₇O₄ |
| 62 | 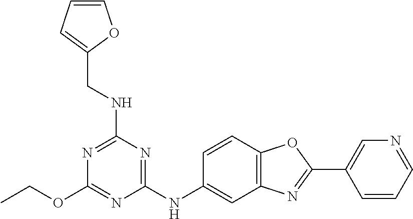 | 6-ethoxy-N-(furan-2- ylmethyl)-N′-[2- (pyridin-3-yl)-1,3- benzoxazol-5-yl]- 1,3,5-triazine-2,4-diamine | 429.4 | C₂₄H₂₁N₇O₂ |
| 63 | 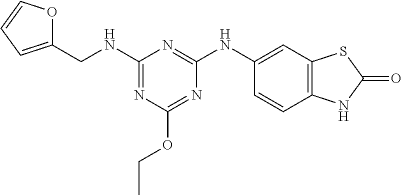 | 6-({4-ethoxy-6- [(furan-2- ylmethyl)amino]- 1,3,5-triazin-2- yl}amino)-1,3- benzothiazol-2(3H)- one | 384.4 | C₂₂H₂₈N₆O₄ |
| 64 | 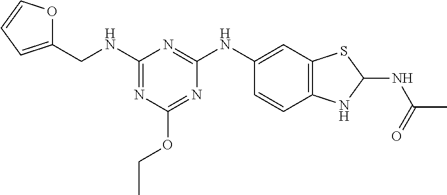 | N-[6-({4-ethoxy- 6-[(furan-2- ylmethyl)amino]- 1,3,5-triazin-2- yl}amino)-1,3- benzothiazol-2- yl]acetamide | 425.5 | C₂₂H₂₃N₅O₃S |
| 65 | 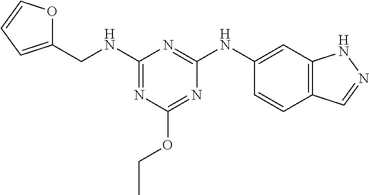 | 6-ethoxy-N-(furan-2- ylmethyl)-N′- (1H-indazol-6-yl)- 1,3,5-triazine-2,4- diamine | 351.4 | C₁₉H₁₉N₇O₃S |
| 66 | 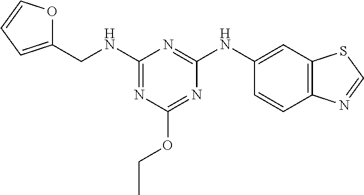 | N-(1,3-benzothiazol- 6-yl)-6-ethoxy- N′-(furan-2-ylmethyl)- 1,3,5-triazine- 2,4-diamine | 368.4 | C₂₅H₂₇N₅O₃ |
| 67 | 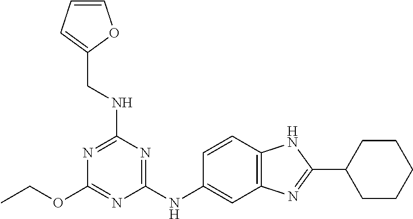 | N-(2-cyclohexyl-1H- benzimidazol-5- yl)-6-ethoxy-N′- (furan-2-ylmethyl)- 1,3,5-triazine-2,4-diamine | 433.5 | C₁₇H₁₇N₇O₂ |
| 68 | 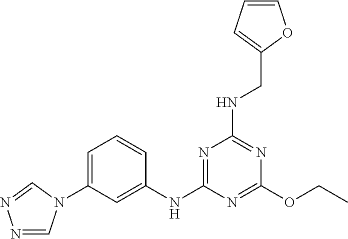 | 6-ethoxy-N-(furan-2- ylmethyl)-N′-[3- (4H-1,2,4-triazol-4- yl)phenyl]-1,3,5- triazine-2,4-diamine | 378.4 | C₂₅H₂₆N₆O₂ |
| 69 | 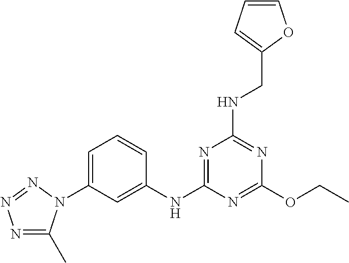 | 6-ethoxy-N-(furan-2- ylmethyl)-N′-[3- (5-methyl-1H-tetrazol- 1-yl)phenyl]- 1,3,5-triazine- 2,4-diamine | 393.4 | C₂₅H₂₄N₆O₃ |
| 70 | 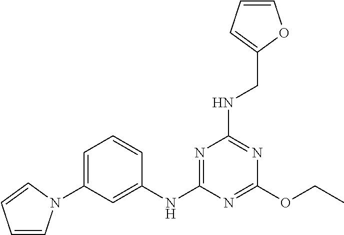 | 6-ethoxy-N-(furan-2- ylmethyl)-N′-[3- (1H-pyrrol-1- yl)phenyl]-1,3,5- triazine-2,4-diamine | 376.4 | C₁₇H₁₇N₅O₄ |
| 71 | 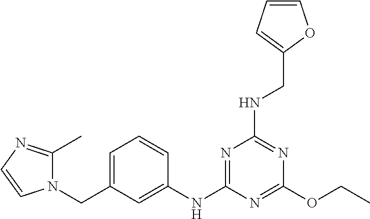 | 6-ethoxy-N-(furan-2- ylmethyl)-N′-{3- [(2-methyl- 1H-imidazol-1- yl)methyl]phenyl}- 1,3,5-triazine-2,4- diamine | 405.5 | C₂₃H₂₀N₆O₂S |
| 72 | 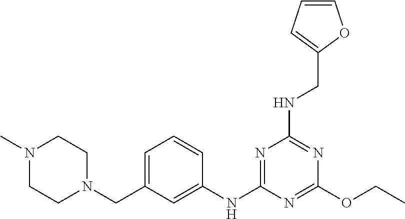 | 6-ethoxy-N-(furan-2- ylmethyl)-N′-{3- [(4-methylpiperazin-1- yl)methyl]phenyl}- 1,3,5-triazine-2,4- diamine | 423.5 | C₂₃H₁₉FN₆O₃ |
| 73 | 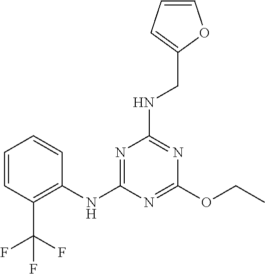 | 6-ethoxy-N-(furan- 2-ylmethyl)-N′-[2- (trifluoromethyl) phenyl]-1,3,5- triazine-2,4-diamine | 379.3 | C₁₇H₁₆N₆O₄ |
| 74 | 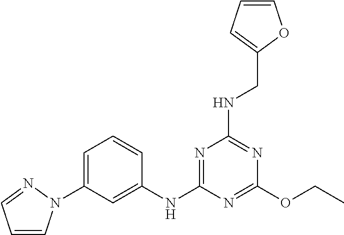 | 6-ethoxy-N-(furan-2- ylmethyl)-N′-[3- (1H-pyrazol-1-yl) phenyl]-1,3,5- triazine-2,4-diamine | 377.4 | C₁₆H₁₆BrN₅O₂ |
| 75 | 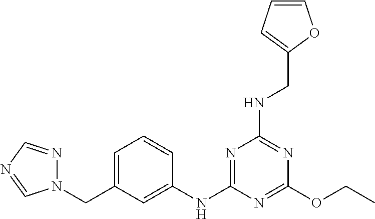 | 6-ethoxy-N-(furan-2- ylmethyl)-N′-[3- (1H-1,2,4-triazol-1- ylmethyl)phenyl]- 1,3,5-triazine- 2,4-diamine | 392.4 | C₂₃H₂₁N₇O₂ |
| 76 | 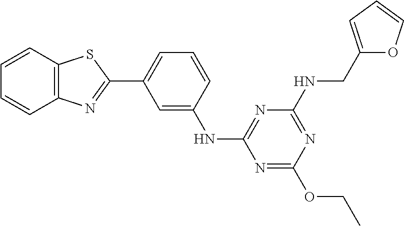 | N-[3-(1,3-benzothiazol- 2-yl)phenyl]- 6-ethoxy-N′-(furan- 2-ylmethyl)- 1,3,5-triazine- 2,4-diamine | 444.5 | C₁₉H₂₄N₆O₃ |
| 77 | 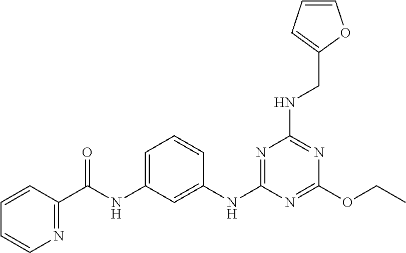 | N-[3-({4-ethoxy- 6-[(furan-2- ylmethyl)amino]- 1,3,5-triazin-2- yl}amino)phenyl] pyridine-2- carboxamide | 431.4 | C₂₀H₂₄N₆O₃ |
| 78 | 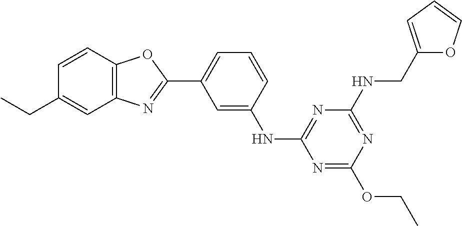 | 6-ethoxy-N-[3- (5-ethyl-1,3- benzoxazol-2-yl) phenyl]-N′-(furan-2- ylmethyl)-1,3,5- triazine-2,4-diamine | 456.5 | C₂₃H₂₄N₆O₂ |
| 79 | 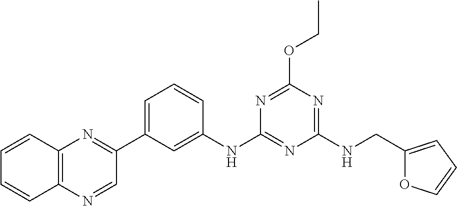 | 6-ethoxy-N-(furan- 2-ylmethyl)-N′-[3- (quinoxalin-2- yl)phenyl]-1,3,5- triazine-2,4-diamine | 439.5 | C₂₉H₃₄N₆O₄ |
| 80 | 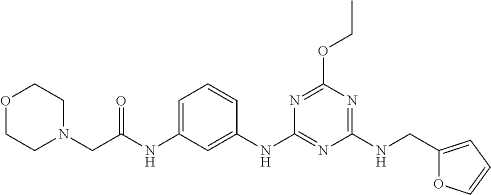 | N-[3-({4-ethoxy- 6-[(furan-2- ylmethyl)amino]- 1,3,5-triazin-2- yl}amino)phenyl]- 2-(morpholin-4- yl)acetamide | 453.5 | C₁₈H₁₈N₆O₃ |
| 81 | 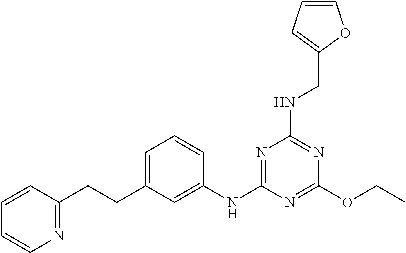 | 6-ethoxy-N-(furan- 2-ylmethyl)-N′-{3- [2-(pyridin-2-yl) ethyl]phenyl}-1,3,5- triazine-2,4-diamine | 416.5 | C₁₇H₁₆N₆O₃S |
| 82 | 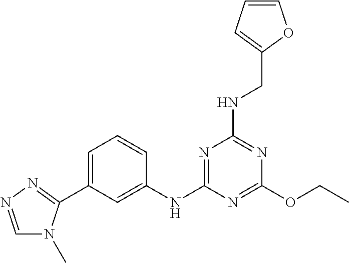 | 6-ethoxy-N-(furan-2- ylmethyl)-N′-[3- (4-methyl-4H- 1,2,4-triazol-3- yl)phenyl]-1,3,5- triazine-2,4-diamine | 392.4 | C₁₇H₁₆F₃N₅O₂ |
| 83 | 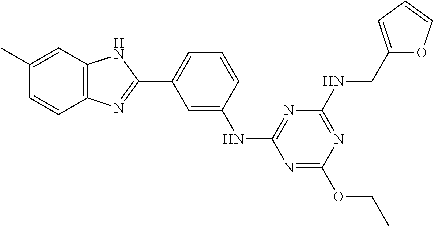 | 6-ethoxy-N-(furan- 2-ylmethyl)-N′-[3- (5-methyl- 1H-benzimidazol-2- yl)phenyl]-1,3,5- triazine-2,4-diamine | 441.5 | C₂₀H₂₀N₆O₂ |
| 84 | 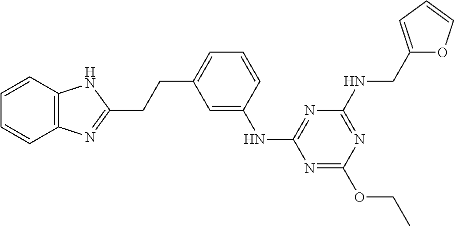 | N-{3-[2-(1H- benzimidazol-2- yl)ethyl]phenyl}- 6-ethoxy-N′-(furan- 2-ylmethyl)- 1,3,5-triazine-2,4- diamine | 455.5 | C₂₄H₂₃N₇O₂ |
| 85 | 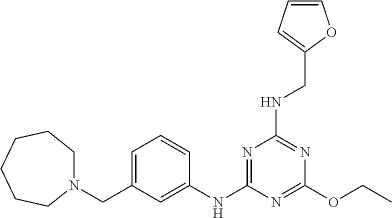 | N-[3-(azepan-1- ylmethyl)phenyl]-6- ethoxy-N′-(furan-2- ylmethyl)-1,3,5- triazine-2,4-diamine | 422.5 | C₁₉H₂₀N₈O₂ |
| 86 | 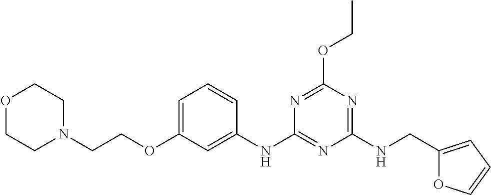 | 6-ethoxy-N-(furan- 2-ylmethyl)-N′-{3- [2-(morpholin-4- yl)ethoxy]phenyl}- 1,3,5-triazine-2,4- diamine | 440.5 | C₁₈H₁₈N₆O₃ |
| 87 | 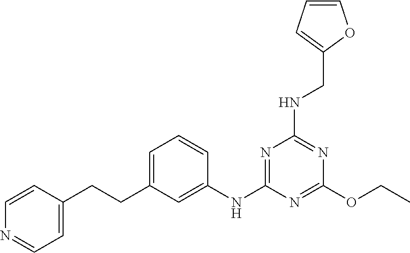 | 6-ethoxy-N-(furan- 2-ylmethyl)-N′-{3- [2-(pyridin-4-yl) ethyl]phenyl}-1,3,5- triazine-2,4-diamine | 416.5 | C₂₅H₂₅N₇O₂ |
| 88 | 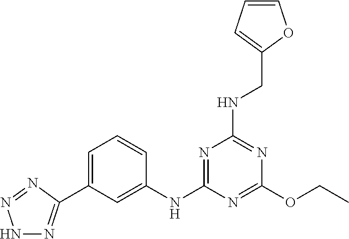 | 6-ethoxy-N-(furan- 2-ylmethyl)-N′-[3- (2H-tetrazol-5- yl)phenyl]-1,3,5- triazine-2,4-diamine | 379.4 | C₁₉H₁₉N₇O₃ |
| 89 | 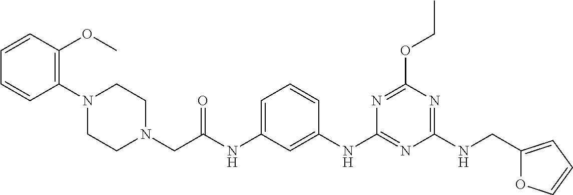 | N-[3-({4-ethoxy-6- [(furan-2- ylmethyl)amino]- 1,3,5-triazin-2- yl}amino)phenyl]- 2-[4-(2- methoxyphenyl) piperazin-1- yl]acetamide | 558.6 | C₁₉H₂₀N₈O₂ |
| 90 | 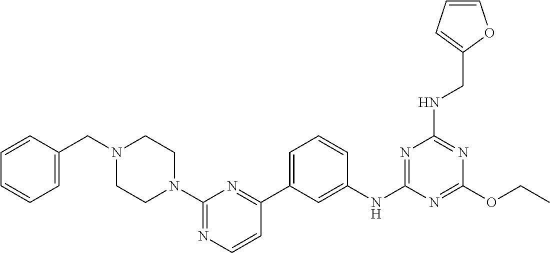 | N-{3-[2-(4- benzylpiperazin-1- yl)pyrimidin-4- yl]phenyl}-6-ethoxy- N′-(furan-2-ylmethyl)- 1,3,5-triazine- 2,4-diamine | 563.7 | C₂₃H₃₀N₆O₂ |
| 91 | 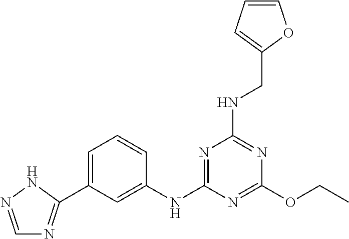 | 6-ethoxy-N-(furan-2- ylmethyl)-N′-[3- (4H-1,2,4-triazol-3- yl)phenyl]-1,3,5- triazine-2,4-diamine | 378.4 | C₁₉H₁₉N₇O₄ |
| 92 | 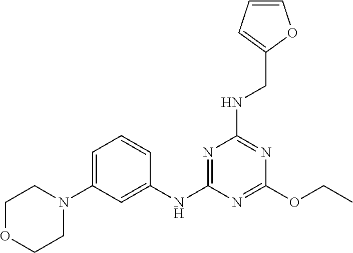 | 6-ethoxy-N-(furan-2- ylmethyl)-N′-[3- (morpholin-4- yl)phenyl]-1,3,5- triazine-2,4-diamine | 396.4 | C₂₂H₂₉N₇O₂ |
| 93 | 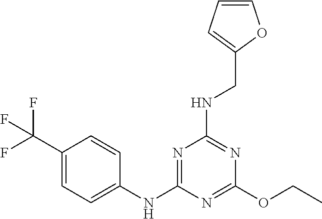 | 6-ethoxy-N-(furan- 2-ylmethyl)-N′-[4- (trifluoromethyl) phenyl]-1,3,5- triazine-2,4-diamine | 379.3 | C₂₃H₂₄N₆O₂ |
| 94 | 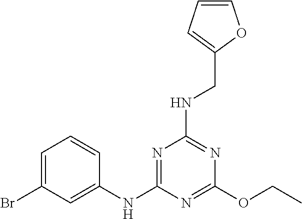 | N-(3-bromophenyl)- 6-ethoxy-N′- (furan-2-ylmethyl)- 1,3,5-triazine- 2,4-diamine | 390.2 | C₂₁H₂₃N₇O₂ |
| 95 | 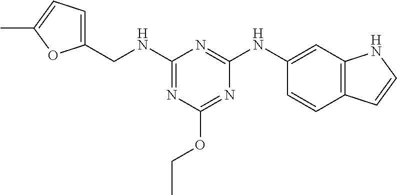 | 6-ethoxy-N-(1H- indol-6-yl)-N′-[(5- methylfuran-2- yl)methyl]-1,3,5- triazine-2,4-diamine | 364.4 | C₁₈H₁₉N₉O₂ |
| 96 | 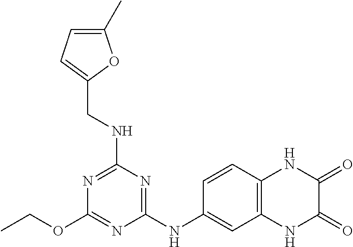 | 6-[(4-ethoxy-6- {[(5-methylfuran-2- yl)methyl]amino}- 1,3,5-triazin-2- yl)amino]-1,4- dihydroquinoxaline- 2,3-dione | 409.4 | C₁₆H₁₆BrN₅O₂ |
| 97 | 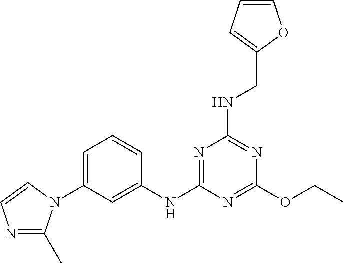 | 6-ethoxy-N-(furan- 2-ylmethyl)-N′-[3- (2-methyl-1H- imidazol-1-yl)phenyl]- 1,3,5-triazine-2,4- diamine | 391.4 | C₁₇H₁₈N₄O₃ |
| 98 | 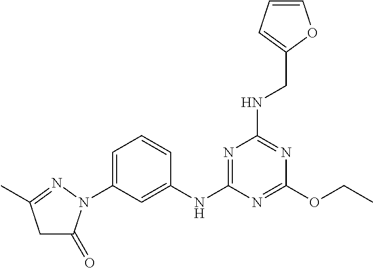 | 2-[3-({4-ethoxy- 6-[(furan-2- ylmethyl)amino]- 1,3,5-triazin-2- yl}amino)phenyl]- 5-methyl-2,4- dihydro-3H- pyrazol-3-one | 407.4 | C₁₉H₂₀N₆O₂ |
| 99 | 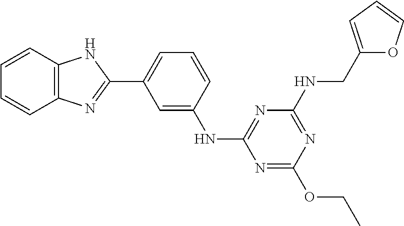 | N-[3-(1H-benzimidazol- 2-yl)phenyl]- 6-ethoxy-N′-(furan- 2-ylmethyl)- 1,3,5-triazine-2,4- diamine | 427.5 | C₂₂H₁₉N₇O₃ |
| 100 | 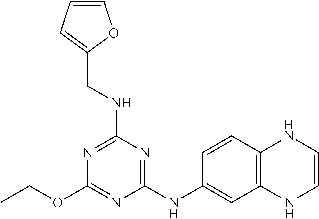 | 6-ethoxy-N-(furan- 2-ylmethyl)-N′- (quinoxalin-6-yl)- 1,3,5-triazine-2,4- diamine | 365.4 | C₁₇H₁₇N₉O₂ |
| 101 | 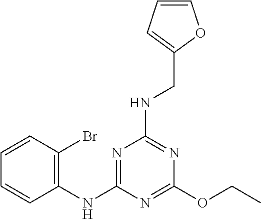 | N-(2-bromophenyl)- 6-ethoxy-N′- (furan-2-ylmethyl)- 1,3,5-triazine- 2,4-diamine | 390.2 | C₂₀H₂₁N₇O₃ |
| 102 | 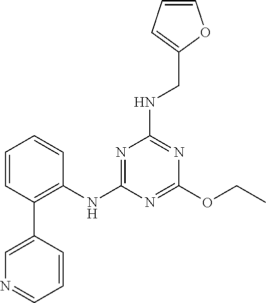 | 6-ethoxy-N-(furan-2- ylmethyl)-N′-[2- (pyridin-3-yl)phenyl]- 1,3,5-triazine- 2,4-diamine | 388.4 | C₁₈H₁₈N₈O₂ |
| 103 | 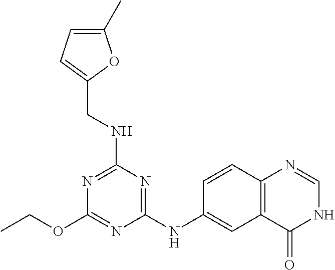 | 6-[(4-ethoxy-6- {[(5-methylfuran-2- yl)methyl]amino}- 1,3,5-triazin-2- yl)amino]quinazolin- 4(3H)-one | 393.4 | C₁₅H₂₂N₆O₂ |
| 104 | 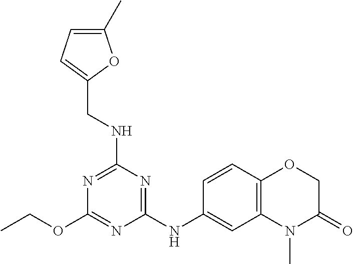 | 6-[(4-ethoxy-6- {[(5-methylfuran-2- yl)methyl]amino}- 1,3,5-triazin-2- yl)amino]-4- methyl-2H-1,4- benzoxazin-3(4H)-one | 410.4 | C₃₁H₃₃N₉O₂ |
| 105 | 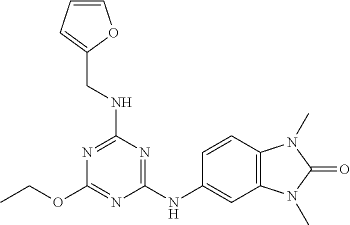 | 5-({4-ethoxy-6- [(furan-2- ylmethyl)amino]- 1,3,5-triazin-2- yl}amino)-1,3-dimethyl- 1,3-dihydro- 2H-benzimidazol-2-one | 395.4 | C₁₇H₁₈N₄O₃ |
| 106 | 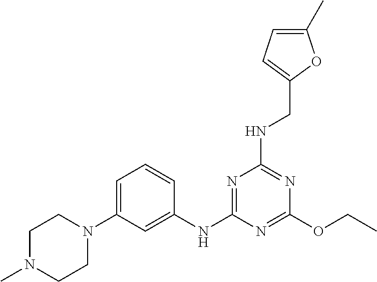 | 6-ethoxy-N- [(5-methylfuran-2- yl)methyl]-N′-[3- (4-methylpiperazin- 1-yl)phenyl]- 1,3,5-triazine-2,4- diamine | 423.5 | C₁₈H₁₈Cl₂N₆O₃ |
| 107 | 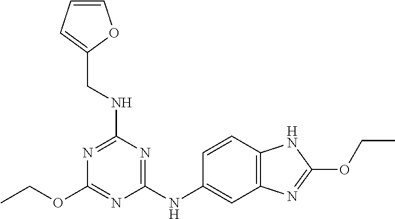 | 6-ethoxy-N- (2-ethoxy-1H- benzimidazol-6- yl)-N′-(furan-2- ylmethyl)-1,3,5- triazine-2,4-diamine | 395.4 | C₁₇H₂₀N₆O₂ |
| 108 | 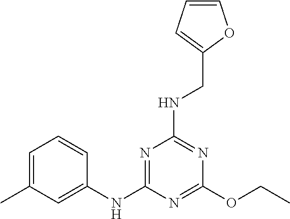 | 6-ethoxy-N-(furan- 2-ylmethyl)-N′-(3- methylphenyl)- 1,3,5-triazine-2,4- diamine | 325.4 | C₁₇H₁₉N₅O₂ |
| 109 | 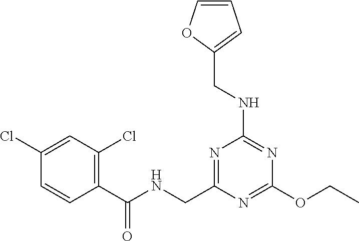 | 2,4-dichloro-N- ({4-ethoxy-6-[(furan- 2-ylmethyl)amino]- 1,3,5-triazin-2- yl}methyl)benzamide | 422.3 | C21H27N7O2 |

### Procedures and Analytical Data for Compounds in Table 9.

### 1. 2-(4-Hydroxy-phenylamino)-4-Ethoxy-6-[(furan-2-ylmethyl)-amino]-[1,3,5]triazine

Sodium (15 mg, 0.63 mmol) was dissolved in ethanol (0.3 mL). The obtained solution was added dropwise at room temperature to a solution of I-45 (200 mg, 0.63 mmol) in ethanol (2 mL). The resulting mixture was stirred at room temperature for 1 hour, then at refluxing for 4 hours (TLC control), cooled down to room temperature, concentrated at reduced pressure. Purification by column chromatography on silica gel (ethyl acetate/hexane) furnished the product (72 mg, 35%).

¹H-NMR (400 MHz, DMSO-D₆) δ_{H}: 1.24 (3H, t, J=7.5 Hz), 4.24 (2H, broad), 4.46 (2H, broad), 6.22 (1H, broad), 6.36 (1H, broad), 6.76 (2H, d, J=8.5 Hz), 7.42 (2H, d, J=8.5 Hz, broad), 7.51 (1H, d, J=1.8 Hz), 7.62 (1H, broad), 8.94 (1H, s), 9.00-9.50 (1H, broad, Z/E forms). LCMS t_{R} (min) 1.50. MS (APCI), m/z 327.70 [M+H]⁺. Mₚ 45° C.

### 2. N-(4-Dimethylamino-phenyl)-6-ethoxy-N′-furan-2-ylmethyl-[1,3,5]triazine-2,4-diamine

¹H-NMR (400 MHz, DMSO-D₆) δ_{H}: 1.25 (3H, t, J=7.5 Hz), 2.80 (6H, s), 4.26 (2H, q, J=7.5 Hz), 4.47 (2H, d, J=7.5 Hz), 6.21 (1H, dd, J=3.6, 1.8 Hz), 6.35 (1H, d, J=3.6 Hz), 6.64 (2H, d, J=8.5 Hz), 7.48 (2H, d, J=8.5 Hz), 7.53 (1H, d, J=1.8 Hz), 7.50-8.10 (1H, broad, Z/E forms), 8.90-9.10 (1H, broad, Z/E forms). LCMS t_{R} (min) 1.37. MS (APCI), m/z 354.73 [M+H]⁺. Mₚ 49-51° C.

### 3. 6-Ethoxy-N-furan-2-ylmethyl-N′-(4-pyridin-4-yl-phenyl)-[1,3,5]triazine-2,4-diamine

¹H-NMR (400 MHz, DMSO-D₆) δ_{H}: 1.30 (3H, t, J=7.5 Hz), 4.32 (2H, broad), 4.52 (2H, broad), 6.28 (1H, broad), 6.39 (1H, d, J=3.6 Hz), 7.56 (1H, d, J=1.8 Hz), 7.67 (2H, d, J=5.0 Hz), 7.73 (2H, broad), 7.90 (3H, broad), 8.58 (2H, d, J=5.0 Hz), 9.50-9.70 (1H, broad, Z/E forms). LCMS t_{R} 1.51 (min). MS (APCI), m/z 389.09 [M+H]⁺. Mₚ 207-209° C.

### 4. 6-Ethoxy-N-furan-2-ylmethyl-N′-(4-pyridin-3-yl-phenyl)[1,3,5]-triazine-2,4-diamine

¹H-NMR (400 MHz, DMSO-D₆) δ_{H}: 1.30 (3H, t, J=7.5 Hz), 4.32 (2H, broad), 4.52 (2H, broad), 6.28 (1H, broad), 6.39 (1H, d, J=3.6 Hz), 7.42 (1H, dd, J=8.0, 5.0 Hz), 7.55 (1H, d, J=1.8 Hz), 7.64 (2H, d, J=8.5 Hz), 7.81 (1H, broad), 7.88 (2H, broad), 8.50 (2H, d, J=5.0 Hz), 8.88 (1H, d, J=1.5 Hz), 9.40-9.60 (1H, broad, Z/E forms). LCMS t_{R} 1.50 (min). MS (APCI), m/z 389.00 [M+H]⁺. Mₚ 181-183° C.

### 5. 6-Ethoxy-N-furan-2-ylmethyl-N′-(4-methoxy-phenyl)-[1,3,5]triazine-2,4-diamine

¹H-NMR (400 MHz, DMSO-D₆) δ_{H}: 1.24 (3H, t, J=7.5 Hz), 3.70 (3H, s), 4.23 (2H, q, J=J=7.5 Hz, broad), 4.46 (2H, d, J=7.6 Hz, broad), 6.20 (1H, dd, J=1.8, 1.2 Hz), 6.38 (1H, dd, 3.8, 1.6 Hz), 6.83 (2H, d, J=8.5 Hz), 7.50-7.65 (3H, m), 7.76 (1H, broad), 9.00-9.35 (1H, broad, Z/E forms). LCMS t_{R} (min) 1.76. MS (APCI), m/z 341.76 [M+H]⁺.

### 6. 6-Ethoxy-N-furan-2-ylmethyl-N′-(3-methoxy-phenyl)-[1,3,5]triazine-2,4-diamine

¹H-NMR (400 MHz, DMSO-D₆) δ_{H}: 1.29 (3H, t, J=7.5 Hz), 3.71 (3H, s), 4.25-4.55 (2H, broad, Z/E forms), 4.53 (2H, broad, Z/E forms), 6.22-6.34 (1H, broad, Z/E forms), 6.34-6.42 (1H, broad, Z/E forms), 6.52-6.63 (1H, broad, two “d”, J=8.5 Hz, Z/E forms), 7.10-7.30 (2H, m), 7.39-7.55 (1H, broad, Z/E forms), 7.52-7.60 (1H, two “s”, Z/E forms), 7.79 (1H, broad), 9.20-9.40 (1H, broad, Z/E forms). LCMS t_{R} (min): 1.87. MS (APCI), m/z 342.10 [M+H]⁺. HPLC t_{R} (min): 12.87. Mₚ 125-127° C.

### 7. N-(3,4-Dimethoxy-phenyl)-6-ethoxy-N′-furan-2-ylmethyl-[1,3,5]triazine-2,4-diamine

A mixture of compound 2 (254 mg, 1 mmol), 3,4-dimethoxyaniline (153 mg, 1.0 mmol), K₂CO₃ (400 mg, 3 mmol) and DMF (5.0 mL) was stirred at 100° C. for 2 hours (TLC control), cooled down to room temperature, diluted with water (50 mL). The formed solid was collected by filtration. Purification by column chromatography on silica gel (methanol/dichloromethane) furnished the product (60 mg, 16%).

¹H-NMR (400 MHz, DMSO-D₆) δ_{H}: 1.28 (3H, t, J=7.6 Hz), 3.20 (3H, s), 3.60-3.80 (3H, broad, Z/E forms), 4.31 (2H, q, J=7.5 Hz), 4.48 (2H, broad), 6.22 (1H, broad), 6.38 (1H, broad), 6.84 (1H, d, J=8.5 Hz), 7.17 (1H, broad), 7.45-7.55 (2H, m, broad), 7.68 (1H, broad), 9.05-9.25 (1H, broad, Z/E forms). LCMS t_{R} 1.68 (min). MS (APCI), m/z 371.98 [M+H]⁺. Mₚ 100-102° C.

### 8. 2-(4-benzamideamino)-4-Ethoxy-6-[(furan-2-ylmethyl)-amino]-[1,3,5]triazine

¹H-NMR (400 MHz, DMSO-D₆) δ_{H}: 1.25 (3H, t, J=7.5), 4.29 (2H, broad), 4.50 (2H, broad), 6.25 (1H, broad), 6.37 (1H, broad), 6.53 (2H, d, J=8.5 Hz), 6.90-7.10 (1H, broad, Z/E forms), 7.56 (2H, d, J=8.5 Hz), 7.70-7.90 (3H, m), 9.50-9.70 (1H, broad, Z/E forms). LCMS t_{R} (min) 2.07. MS (APCI), m/z 354.97 [M+H]⁺. Mₚ 130-132° C.

### 9. 6-Ethoxy-N-furan-2-ylmethyl-N′-(2-methyl-benzothiazol-6-yl)-[1,3,5]triazine-2,4-diamin

¹H-NMR (400 MHz, DMSO-D₆) δ_{H}: 1.30 (3H, t, J=7.5 Hz), 2.75 (3H, s), 4.32 (2H, broad), 4.51 (2H, broad), 6.26 (1H, broad), 6.38 (1H, broad), 7.56 (1H, s), 6.65 (1H, d, J=8.5 Hz), 7.76 (1H, d, J=8.5 Hz), 7.97 (1H, broad), 8.40-8.70 (1H, broad, Z/E forms), 9.50-9.70 (1H, broad, Z/E forms). LCMS t_{R} 1.74 (min). MS (APCI), m/z 382.98 [M+H]⁺. Mₚ 96-98° C.

### 10. 6-Ethoxy-N-furan-2-ylmethyl-N′-(2-methyl-3H-benzoimidazol-5-yl)-[1,3,5]triazine-2,4-diamine

A mixture of compound 2 (254 mg, 1 mmol), aniline (150 mg, 1.0 mmol), K₂CO₃ (400 mg, 3 mmol) and DMSO (5.0 mL) was stirred at 100° C. for 2 hours (TLC control), cooled down to room temperature, diluted with water (50 mL). The formed solid was collected by filtration. Purification by column chromatography on silica gel (methanol/dichloromethane) furnished the product (40 mg, 11%)

¹H-NMR (400 MHz, DMSO-D₆) δ_{H}: 1.28 (3H, t, J=7.5 Hz), 2.42 (3H, s), 4.30 (2H, broad, Z/E forms), 4.50 (2H, broad, Z/E forms), 6.22 (1H, broad, Z/E forms), 6.36 (1H, broad, Z/E forms), 7.35-7.40 (2H, m), 7.53 (1H, s), 7.67 (1H, broad, Z/E forms), 7.80-8.05 (1H, broad, Z/E forms), 9.10-9.35 (1H, broad, Z/E forms), 12.10 (1H, broad). LCMS t_{R} 1.37 (min). MS (APCI), m/z 366.11 [M+H]⁺. Mₚ 150-152° C.

### 11. 6-Ethoxy-N-furan-2-ylmethyl-N′-(2-piperidin-1-ylmethyl-3H-benzoimidazol-5-yl)-[1,3,5]triazine-2,4-diamine

¹H-NMR (400 MHz, DMSO-D₆) δ_{H}: 1.25 (3H, t, J=7.5 Hz), 1.30-1.60 (6H, m), 2.40 (4H, m), 3.59 (2H, s), 4.28 (2H, broad, Z/E forms), 4.48 (2H, broad, Z/E forms), 6.21 (1H, broad, Z/E forms), 6.33 (1H, broad, Z/E forms), 7.33 (2H, broad, Z/E forms), 7.52 (1H, s), 7.65 (1H, s, broad), 7.98 (1H, broad, Z/E forms), 9.10-9.35 (1H, broad, Z/E forms), 11.96 (1H, broad). LCMS t_{R} 1.34 (min). MS (APCI), m/z 449.02 [M+H]⁺. Mₚ 155-157° C.

### 12. 5-{4-Ethoxy-6-[(furan-2-ylmethyl)-amino]-[1,3,5]triazin-2-ylamino}-1,3-dihydro-benzoimidazol-2-one

¹H-NMR (400 MHz, DMSO-D₆) δ_{H}: 1.27 (3H, t, J=7.5 Hz), 4.28 (2H, broad, Z/E forms), 4.48 (2H, broad, Z/E forms), 6.23 (1H, broad, Z/E forms), 6.37 (1H, broad, Z/E forms), 6.79 (1H, d, J=8.5 Hz), 7.20 (1H, broad peak, Z/E forms), 7.25-7.70 (2H, m), 7.53 (1H, d, J=1.5 Hz), 9.18-9.30 (1H, broad, Z/E forms), 10.31 (1H, s), 10.44 (1H, broad, Z/E forms). LCMS t_{R} 1.38 (min). MS (APCI), m/z 368.08 [M+H]⁺. Mₚ 198-200° C.

### 13. 5-{4-Ethoxy-6-[(furan-2-ylmethyl)-amino]-[1,3,5]triazin-2-ylamino}-1,3-dihydro-indol-2-one

¹H-NMR (400 MHz, DMSO-D₆) δ_{H}: 1.25 (3H, t, J=7.5 Hz), 3.41 (2H, s), 4.25 (2H, broad, Z/E forms), 4.49 (2H, broad), 6.22 (1H, broad, Z/E forms), 6.38 (1H, broad, Z/E forms), 6.71 (1H, d, J=8.5 Hz), 7.44 (1H, d, J=8.5 Hz), 7.54 (1H, s), 7.58 (1H, s, broad), 7.67 (1H, broad, Z/E forms), 9.05-9.30 (1H, broad, Z/E forms), 10.12 (1H, s). LCMS t_{R} 1.42 (min). MS (APCI), m/z 367.03 [M+H]⁺. Mₚ 260-262° C. (decomp)

### 14. 6-Ethoxy-N-furan-2-ylmethyl-N′-(1H-indol-5-yl)-[1,3,5]triazine-2,4-diamine

¹H-NMR (400 MHz, DMSO-D₆) δ_{H}: 1.28 (3H, t, J=7.5 Hz), 4.28 (2H, q, J=7.5 Hz), 4.48 (2H, broad, Z/E forms), 6.23 (1H, broad, Z/E forms), 6.33 (1H, s), 6.37 (1H, broad), 7.24-7.33 (2H, m), 7.54 (1H, s), 7.63 (1H, broad), 7.88 (1H, s), 8.95-9.20 (1H, broad, Z/E forms), 10.83 (1H, broad). LCMS t_{R} 1.58 (min). MS (APCI), m/z 351.01 [M+H]⁺. Mₚ 108-110° C.

### 15. 6-Ethoxy-N-furan-2-ylmethyl-N′-(1H-indol-6-yl)-[1,3,5]triazine-2,4-diamine

¹H-NMR (400 MHz, DMSO-D₆) δ_{H}: 1.30 (3H, t, J=7.5 Hz), 4.31 (2H, q, J=7.5 Hz), 4.47-4.65 (2H, broad, Z/E forms), 6.27 (1H, broad, Z/E forms), 6.33 (1H, s), 6.38 (1H, broad), 7.18-7.28 (2H, broad), 7.40 (1H, d, J=8.5 Hz), 7.55 (1H, s), 7.65-7.98 (2H, broad, Z/E forms), 9.20-9.50 (1H, broad, Z/E forms), 10.91 (1H, broad). LCMS t_{R} 1.65 (min). MS (APCI), m/z 351.08 [M+H]⁺. Mₚ 112-114° C.

### 16. 6-Ethoxy-N-furan-2-ylmethyl-N′-(1H-indazol-5-yl)-[1,3,5]triazine-2,4-diamine

¹H-NMR (400 MHz, DMSO-D₆) δ_{H}: 1.25 (3H, t, J=7.5 Hz), 4.28 (2H, broad, Z/E forms), 4.45 (2H, broad, Z/E forms), 6.23 (1H, broad, Z/E forms), 6.36 (1H, broad, Z/E forms), 6.40 (1H, d, J=8.5 Hz), 7.53 (2H, broad), 7.71 (1H, broad, Z/E forms), 8.10 (1H, s), 8.13 (1H, broad), 9.20-9.40 (1H, broad, Z/E forms), 12.81 (1H, s). LCMS t_{R} 1.51 (min). MS (APCI), m/z 352.01 [M+H]⁺. Mₚ 248-250° C. (decomp)

### 17. 6-{4-Ethoxy-6-[(furan-2-ylmethyl)-amino]-[1,3,5]triazin-2-ylamino}-3H-benzooxazol-2-one

Yield 35 mg, 5%. ¹H-NMR (400 MHz, DMSO-D₆) δ_{H}: 1.28 (3H, t, J=7.5 Hz), 4.30 (2H, broad), 4.50 (2H, broad), 6.25 (1H, broad), 6.38 (1H, broad), 6.97 (1H, d, J=8.5 Hz), 7.32 (1H, broad doublet), 7.54 (1H, s), 7.78-8.00 (1H, broad, Z/E forms), 7.82 (1H, broad), 9.32-9.48 (1H, broad, Z/E forms), 11.37 (1H, broad). LCMS t_{R} (min): 1.58. MS (APCI), m/z 369.05 [M+H]⁺. HPLC t_{R} (min): 9.94. Mₚ 254-256° C.

### 18. 5-{4-Ethoxy-6-[(furan-2-ylmethyl)-amino]-[1,3,5]triazin-2-ylamino}-isoindole-1,3-dione

¹H-NMR (400 MHz, DMSO-D₆) δ_{H}: 1.31 (3H, t, J=7.5 Hz), 4.35 (2H, broad), 4.52 (2H, broad), 6.22-6.40 (1H, broad, Z/E forms), 6.39 (1H, broad), 7.56 (1H, s), 7.70 (1H, broad), 8.04 (1H, broad), 8.00-8.19 (1H, broad, Z/E forms), 8.23-8.39 (1H, broad, Z/E forms), 9.93-10.12 (1H, broad, Z/E forms), 11.00 (1H, broad). LCMS t_{R} (min): 1.64. MS (APCI), m/z 381.00 [M+H]⁺. HPLC t_{R} (min): 11.63. Mₚ 242-244° C.

### 19. N-(2,3-Dihydro-benzo[1,4]dioxin-6-yl)-6-ethoxy-N′-furan-2-ylmethyl-[1,3,5]triazine-2,4-diamine

¹H-NMR (400 MHz, DMSO-D₆) δ_{H}: 1.27 (3H, t, J=7.5 Hz), 4.19 (4H, broad), 4.28 (2H, broad, Z/E forms), 4.46 (2H, broad, Z/E forms), 6.24 (1H, d, J=8.5 Hz, broad), 6.37 (1H, broad), 6.72 (1H, d, J=8.5 Hz), 7.19 (1H, broad), 7.38 (1H, broad), 7.54 (1H, s), 7.76 (1H, broad), 9.05-9.30 (1H, broad, Z/E forms). LCMS t_{R} 1.67 (min). MS (APCI), m/z 370.01 [M+H]⁺. Mₚ 160-162° C.

### 20. N-Benzo[1,3]dioxol-5-yl-6-ethoxy-N′-furan-2-ylmethyl-[1,3,5]triazine-2,4-diamine

Yield 214 mg, 38%. ¹H-NMR (400 MHz, DMSO-D₆) δ_{H}: 1.29 (3H, t, J=7.5 Hz), 4.30 (2H, broad), 4.48 (2H, broad), 5.95 (2H, s), 6.23 (1H, broad), 6.38 (1H, broad), 6.80 (1H, d, J=8.5 Hz), 7.07 (1H, d, J=8.5 Hz), 7.43-7.52 (1H, broad, Z/E forms), 7.54 (1H, s), 7.76 (1H, broad), 9.15-9.30 (1H, broad, Z/E forms). LCMS t_{R} (min): 1.76. MS (APCI), m/z 356.08 [M+H]⁺. HPLC t_{R} (min): 11.98. Mₚ 89-91° C.

### 21. 7-{4-Ethoxy-6-[(furan-2-ylmethyl)-amino]-[1,3,5]triazin-2-ylamino}-4H-benzo[1,4]oxazin-3-one

A mixture of compound 2 (102 mg, 0.4 mmol), 7-amino-4H-benzo[1,4]oxazin-3-one (66 mg, 0.4 mmol), K₂CO₃ (170 mg, 1.2 mmol) and DMF (2.5 mL) was stirred at 100° C. for 2 hours (TLC control), cooled down to room temperature, diluted with water (30 mL). The formed solid was collected by filtration. Purification by column chromatography on silica gel (methanol/dichloromethane) furnished the product (61 mg, 40%).

¹H-NMR (400 MHz, DMSO-D₆) δ_{H}: 1.36 (3H, t, J=7.5 Hz), 4.28 (2H, q, J=7.5, brs), 4.47 (2H, d, J=7.4, brs), 4.50 (2H, s), 6.24 (1H, dd, J=3.6, 1.8 Hz), 6.35 (1H, d, J=3.6 Hz), 6.77 (1H, d, J=8.5 Hz), 7.23 (1H, d, J=8.5 Hz), 7.44 (1H, dd, J=8.5, 1.7 Hz, broad), 7.52 (1H, d, J=1.8 Hz), 7.68 (1H, broad), 9.23-9.34 (1H, broad, Z/E forms), 10.46 (1H, s). LCMS t_{R} (min) 2.21. MS (APCI), m/z 382.99 [M+H]⁺. Mₚ 270° C. (decomp.)

### 22. 4-{4-Ethoxy-6-[(5-methyl-furan-2-ylmethyl)-amino]-[1,3,5]-triazin-2-ylamino}-phenol

To a solution of I-3 (514 mg, 2.0 mmol) in THF (10 mL) a solution of 5-methylfurfurylamine (222 mg, 2.0 mmol) and N,N-diisopropylethylamine (260 mg, 2.0 mmol) in THF (10 mL) was added slowly dropwise at 0° C. The resulting mixture was stirred at 0° C. for 2 hours and 3 hours at room temperature (TLC control) and concentrated at reduced pressure. Purification by column chromatography on silica gel (ethyl acetate/hexane) gave 4-{4-chloro-6-[(5-methyl-furan-2-ylmethyl)-amino]-[1,3,5]triazin-2-ylamino}-phenol (463 mg, 70%).

Sodium (46 mg, 2.0 mmol) was dissolved in anhydrous ethanol (1 mL) at room temperature. The obtained solution was added dropwise to a solution of 4-{4-Ethoxy-6-[(5-methyl-furan-2-ylmethyl)-amino]-[1,3,5]-triazin-2-ylamino}-phenol (332 mg, 1.0 mmol) in anhydrous ethanol (3 mL). The resulting mixture was stirred at room temperature for 20 minutes, and then at refluxing for 2 hours. After completion of the reaction (TLC control) the solvent was removed at reduced pressure. The reaction mixture was washed with water (10 mL) and extracted with chloroform (3×5 mL). The combined organic phases were concentrated at reduced pressure. Purification by column chromatography (silica gel, methanol/ethyl acetate) gave the product (34 mg, 10%). ¹H-NMR (400 MHz, DMSO-D₆) δ_{H}: 1.23 (3H, t, J=7.5 Hz), 2.19 (3H, s), 4.22 (2H, broad, Z/E forms), 4.36 (2H, d, J=7.5 Hz), 5.91 (1H, broad), 6.05 (1H, broad), 6.63 (2H, d, J=8.5 Hz), 7.41 (2H, d, J=8.5 Hz), 7.54 (1H, broad, Z/E forms), 8.93 (1H, s), 9.15 (1H, broad, Z/E forms). LCMS t_{R} 1.60 (min). MS (APCI), m/z 341.78 [M+H]⁺. Mₚ 40-42° C.

### 23. 6-Ethoxy-N-furan-2-ylmethyl-N′-(4-morpholin-4-yl-phenyl)-[1,3,5]triazine-2,4-diamine

¹H-NMR (400 MHz, DMSO-D₆) δ_{H}: 1.25 (3H, t, J=7.5), 2.96-3.02 (4H, m), 3.69-3.74 (4H, m), 4.25 (2H, q, J=7.5 Hz), 4.45 (2H, d, J=7.4 Hz), 6.21 (1H, dd, J=3.6, 1.8 Hz), 6.35 (1H, d, J=3.6 Hz), 6.85 ((2H, d, J=8.5 Hz), 7.50-7.58 (3H, m), 7.64 (1H, broad), 7.60-8.70 (1H, broad, Z/E forms), 9.00-9.20 (1H, broad, Z/E forms). LCMS t_{R} (min) 1.73. MS (APCI), m/z 396.70 [M+H]⁺.

### 24. N-(3-Dimethylamino-phenyl)-6-ethoxy-N′-furan-2-ylmethyl-[1,3,5]triazine-2,4-diamine

Yield 480 mg, 47%. ¹H-NMR (400 MHz, DMSO-D₆) δ_{H}: 1.30 (3H, t, J=7.5 Hz), 2.86 (6H, s), 4.31 (2H, broad), 4.43-4.59 (2H, broad, Z/E forms), 6.24 (1H, broad), 6.38 (2H, broad), 7.05 (2H, broad multiplet), 7.23-7.31 (1H, broad, Z/E forms), 7.52 (1H, s), 7.70 (1H, broad), 9.01-9.20 (1H, broad, Z/E forms). LCMS t_{R} (min): 1.56. MS (APCI), m/z 355.13 [M+H]⁺. HPLC t_{R} (min): 9.55. Mₚ 162-164° C.

### 25. N-(4-Chloro-phenyl)-6-ethoxy-N′-furan-2-ylmethyl-[1,3,5]triazine-2,4-diamine

¹H-NMR (400 MHz, DMSO-D₆) δ_{H}: 1.26 (3H, t, J=7.6 Hz), 4.28 (2H, q, J=7.5 Hz), 4.47 (2H, d, J=7.5 Hz), 6.22 (1H, broad), 6.37 (1H, broad), 7.28 (2H, d, J=8.5 Hz), 7.53 (1H, d, J=1.8 Hz), 7.65-7.85 (3H, m, broad, Z/E forms), 9.35-9.55 (1H, broad, Z/E forms). LCMS t_{R} 1.99 (min). MS (APCI), m/z 345.67 [M+H]⁺. Mₚ 115-118° C.

### 26. 6-{4-Ethoxy-6-[(furan-2-ylmethyl)-amino]-[1,3,5]triazin-2-ylamino}-3-methyl-3H-benzooxazol-2-one

¹H-NMR (400 MHz, DMSO-D₆) δ_{H}: 1.28 (3H, t, J=7.5 Hz), 3.20 (3H, s), 4.28 (2H, q, J=7.5 Hz, broad), 4.50 (2H, d, J=7.5 Hz, broad), 6.26 (1H, broad), 6.48 (1H, broad), 7.12 (1H, d, J=8.5 Hz), 7.41 (1H, broad), 7.52 (1H, d, J=1.8 Hz), 7.80 (1H, broad), 7.80-8.05 (1H, broad, Z/E forms), 9.30-9.50 (1H, broad, Z/E forms). LCMS t_{R} 1.56 (min). MS (APCI), m/z 382.97 [M+H]⁺. Mₚ 220-222° C.

### 27. 6-(4-Ethoxy-6-[(furan-2-ylmethyl)-amino]-[1,3,5]triazin-2-ylamino}-1,4-dihydro-quinoxaline-2,3-dione

Yield 184 mg, 50%. ¹H-NMR (400 MHz, DMSO-D₆) δ_{H}: 1.30 (3H, t, J=7.5 Hz), 4.30 (2H, broad), 4.52 (2H, broad), 6.25 (1H, broad), 6.39 (1H, broad), 7.01 (1H, d, J=8.5 Hz), 7.36 (1H, broad), 7.53 (1H, s), 7.55 (1H, broad), 7.60-7.78 (1H, broad, Z/E forms), 9.30-9.47 (1H, broad, Z/E forms), 11.80 (2H, broad). LCMS t_{R} (min): 1.47. MS (APCI), m/z 396.05 [M+H]⁺. HPLC t_{R} (min): 8.50. Mₚ 205-208° C.

### 28. 6-Ethoxy-N-furan-2-ylmethyl-N′-(2-pyridin-4-yl-benzooxazol-5-yl)-[1,3,5]triazine-2,4-diamine

¹H-NMR (400 MHz, DMSO-D₆) δ_{H}: 1.30 (3H, t, J=7.5 Hz), 4.31 (2H, q, J=7.5 Hz, broad), 4.53 (2H, broad), 6.25-6.35 (1H, broad, Z/E forms), 6.49 (1H, broad), 7.56 (1H, s), 7.72 (2H, m), 7.80-8.00 (1H, broad, Z/E forms), 8.08 (2H, d, J=5.0 Hz), 8.35-8.55 (1H, broad, Z/E forms), 8.83 (2H, d, J=5.0 Hz), 9.50-9.70 (1H, broad, Z/E forms). LCMS t_{R} 1.67 (min). MS (APCI), m/z 430.03 [M+H]⁺. Mₚ 220-202° C. (decomp.)

### 29. 6-Ethoxy-N-furan-2-ylmethyl-N′-(2-methyl-benzothiazol-5-yl)-[1,3,5]triazine-2,4-diamine

¹H-NMR (400 MHz, DMSO-D₆) δ_{H}: 1.29 (3H, t, J=7.5 Hz), 2.79 (3H, s), 4.31 (2H, broad), 4.51 (2H, broad), 6.18-6.40 (1H, broad, Z/E forms), 6.38 (1H, broad), 7.55 (1H, broad), 7.69 (1H, d, J=8.5 Hz), 7.85 (2H, m), 8.48 (1H, broad), 9.45-9.65 (1H, broad, Z/E forms). LCMS t_{R} (min): 1.81. MS (APCI), m/z 383.07 [M+H]⁺. HPLC t_{R} (min): 12.32.

### 30. 6-Ethoxy-N-furan-2-ylmethyl-N′-(2-methyl-benzooxazol-6-yl)-[1,3,5]triazine-2,4-diamine

Yield 243 mg, 25%. ¹H-NMR (400 MHz, DMSO-D₆) δ_{H}: 1.30 (3H, t, J=7.5 Hz), 2.57 (3H, s), 4.32 (2H, broad), 4.51 (2H, broad), 6.28 (1H, broad), 6.38 (1H, broad), 7.49 (2H, m), 7.53 (1H, s), 7.85 (1H, broad), 8.20-8.40 (1H, broad, Z/E forms), 9.45-9.62 (1H, broad, Z/E forms). LCMS t_{R} (min): 1.69. MS (APCI), m/z 367.07 [M+H]⁺. HPLC t_{R} (min): 11.67. Mₚ 80-82° C.

### 31. 6-Ethoxy-N-furan-2-ylmethyl-N′-(3-trifluoromethyl-phenyl)-[1,3,5]triazine-2,4-diamine

¹H-NMR (400 MHz, DMSO-D₆) δ_{H}: 1.30 (3H, t, J=7.5 Hz), 4.30 (2H, broad), 4.51 (2H, broad), 6.22 (1H, broad), 6.37 (1H, broad), 7.28 (1H, d, J=8.5 Hz), 7.46 (1H, t, J=8.5 Hz), 7.50 (1H, s), 7.80-8.10 (2H, broad, Z/E forms), 8.10-8.40 (1H, broad, Z/E forms), 9.55-9.80 (1H, broad, Z/E forms). LCMS t_{R} 2.00 (min). MS (APCI), m/z 379.95 [M+H]⁺. Mₚ 147-149° C.

### 32. 3-{4-Ethoxy-6-[(furan-2-ylmethyl)-amino]-[1,3,5]triazin-2-ylamino}-benzonitrile

¹H-NMR (400 MHz, DMSO-D₆) δ_{H}: 1.30 (3H, t, J=7.5 Hz), 4.32 (2H, broad), 4.51 (2H, d, J=7.5 Hz), 6.28 (1H, dd, J=3.6, 1.8 Hz), 6.39 (1H, d, J=3.6 Hz), 7.39 (1H, d, J=8.5 Hz), 7.46 (1H, t, J=8.5 Hz), 7.55 (1H, d, J=1.8 Hz), 7.96 (2H, broad), 8.20-8.40 (1H, broad, Z/E forms), 9.60-9.80 (1H, broad, Z/E forms). LCMS t_{R} 1.83 (min). MS (APCI), m/z 336.94 [M+H]⁺. Mₚ 164-166° C.

### 33. 6-Ethoxy-N-furan-2-ylmethyl-N′-(3-phenoxy-phenyl)-[1,3,5]triazine-2,4-diamine

¹H-NMR (400 MHz, DMSO-D₆) δ_{H}: 1.22 (3H, t, J=7.5 Hz), 4.21 (2H, broad), 4.30-4.50 (2H, broad, Z/E forms), 6.21 (1H, broad), 6.34 (1H, broad), 6.61 (1H, broad), 7.00 (2H, d, J=8.5 Hz), 7.11 (1H, broad), 7.25 (1H, broad), 7.38 (2H, broad), 7.51 (2H, broad), 7.55-7.70 (1H, broad, Z/E forms), 7.77 (1H, broad), 9.35-9.55 (1H, broad, Z/E forms). LCMS t_{R} 2.05 (min). MS (APCI), m/z 404.02 [M+H]⁺. Mₚ 145-147° C.

### 34. (3-{4-Ethoxy-6-[(furan-2-ylmethyl)-amino]-[1,3,5]triazin-2-ylamino}-phenyl)-piperidin-1-yl-methanone

¹H-NMR (400 MHz, DMSO-D₆) δ_{H}: 1.24 (3H, t, J=7.5 Hz), 1.35-1.65 (6H, m), 3.20-3.60 (4H, m, broad), 4.28 (2H, broad, Z/E forms), 4.49 (2H, broad, Z/E forms), 6.22 (1H, broad, Z/E forms), 6.35 (1H, broad, Z/E forms), 6.92 (1H, d, J=8.5 Hz), 7.28 (1H, t, J=8.5 Hz), 7.51 (1H, s), 7.60-7.90 (3H, broad, Z/E forms), 9.35-9.55 (1H, broad, Z/E forms). LCMS t_{R} 1.72 (min). MS (APCI), m/z 422.85 [M+H]⁺. Mₚ 146-148° C.

### 35. 6-Ethoxy-N-furan-2-ylmethyl-N′-(3-piperidin-1-ylmethyl-phenyl)-[1,3,5]triazine-2,4-diamine

¹H-NMR (400 MHz, DMSO-D₆) δ_{H}: 1.26 (3H, t, J=7.5 Hz), 1.33 (2H, broad), 1.47 (4H, broad), 2.30 (4H, broad), 3.34 (2H, broad), 4.31 (2H, d, J=7.5 Hz), 4.50 (2H, broad), 6.25 (1H, broad), 6.36 (1H, broad), 6.90 (1H, d, J=8.5 Hz), 7.18 (1H, t, J=8.5 Hz), 7.50-7.90 (3H, m), 7.53 (1H, s), 9.15-9.40 (1H, broad, Z/E forms). LCMS t_{R} 1.39 (min). MS (APCI), m/z 409.06 [M+H]⁺.

### 36. 6-Ethoxy-N-furan-2-ylmethyl-N′-(1-methyl-1H-benzoimidazol-5-yl)-[1,3,5]triazine-2,4-diamine

A mixture of 2 (508 mg, 2 mmol), aniline (294 mg, 2 mmol), KOH (168 mg, 3 mmol) and acetone (5 mL) was stirred at refluxing for 20 hours, diluted with water, extracted with ethyl acetate. The combined organic phases were concentrated. Purification by column chromatography (silica gel, methanol/ethyl acetate) gave the product (70 mg, 10%)

¹H-NMR (400 MHz, DMSO-D₆) δ_{H}: 1.28 (3H, t, J=7.5 Hz), 3.80 (3H, s), 4.30 (2H, q, J=7.5 Hz), 4.48 (2H, broad), 6.20-6.40 (1H, broad, Z/E forms), 6.38 (1H, broad), 7.44 (1H, J=8.5 Hz), 7.53 (1H, broad), 7.55 (1H, d, J=1.8 Hz), 7.72 (1H, broad), 8.10 (1H, broad), 8.12 (1H, s), 9.15-9.40 (1H, broad, Z/E forms). LCMS t_{R} 1.38 (min). MS (APCI), m/z 366.02 [M+H]⁺. Mₚ 105-107° C.

### 37. N-(1H-Benzoimidazol-5-yl)-6-ethoxy-N′-furan-2-ylmethyl-[1,3,5]triazine-2,4-diamine

A mixture of compound 2 (300 mg, 1.17 mmol), aniline dihydrochloride (240 mg, 1.17 mmol), K₂CO₃ (815 mg, 5.85 mmol) and DMSO (0.5 mL) was stirred at 150° C. for 4 hours, cooled down to room temperature, diluted with water, extracted with chloroform. The combined organic phases were concentrated at reduced pressure. Purification by column chromatography (silica gel, ethyl acetate/methanol, 3 times) and preparative TLC gave the product (33 mg, 8%).

¹H-NMR (400 MHz, DMSO-D₆) δ_{H}: 1.32 (3H, t, J=7.5 Hz), 4.43 (2H, q, J=7.5 Hz), 4.50-4.70 (2H, broad, Z/E forms), 5.02 (1H, broad), 5.15 (1H, broad), 6.25-6.40 (2H, m), 6.52 (1H, broad), 7.34 (1H, d, J=8.5 Hz), 7.54 (1H, s), 7.61-7.72 (1H, broad, Z/E forms), 8.50 (1H, broad), 8.55-8.70 (1H, broad, Z/E forms). LCMS t_{R} (min): 1.47. MS (APCI), m/z 352.13 [M+H]⁺. HPLC t_{R} (min): 9.20. Mₚ 110-112° C.

### 38. 6-Ethoxy-N-furan-2-ylmethyl-N′-(2-thiophen-2-yl-1H-benzoimidazol-5-yl)-[1,3,5]triazine-2,4-diamine

A mixture of compound 2 (255 mg, 1.0 mmol), 2-thiophen-2-yl-1H-benzoimidazol-5-ylamine (215 mg, 1.0 mmol), DIPEA (194 mg, 1.5 mmol) and dioxane (5 mL) was stirred at 70° C. for 8 hours, cooled down to room temperature. Then the obtained mixture was transferred to column (silica gel, methanol/ethyl acetate). Additional purification by preparative TLC (methanol/ethyl acetate) gave the product (38 mg, 9%).

¹H-NMR (400 MHz, DMSO-D₆) δ_{H}: 1.29 (3H, broad), 4.28 (2H, broad), 4.51 (2H, broad), 6.24 (1H, broad), 6.36 (1H, broad), 7.18 (1H, broad), 7.30-7.50 (1H, broad, Z/E forms), 7.50-7.55 (1H, broad, Z/E forms), 7.64 (2H, broad), 7.73 (2H, broad), 7.90-8.20 (1H, broad, Z/E forms) 9.10-9.45 (1H, broad, Z/E forms), 12.66 (1H, s).

LCMS t_{R} (min): 1.54. MS (APCI), m/z 434.13 [M+H]⁺. HPLC t_{R} (min): 9.93. Mₚ 170-172° C.

### 39. 6-Ethoxy-N-furan-2-ylmethyl-N′-(1-methyl-2-morpholin-4-ylmethyl-1H-benzoimidazol-5-yl)-[1,3,5]triazine-2,4-diamine

¹H-NMR (400 MHz, DMSO-D₆) δ_{H}: 1.28 (3H, t, J=7.5 Hz), 2.42 (4H, m), 3.55 (4H, m), 3.75 (2H, s), 3.80 (3H, s), 4.30 (2H, q, J=7.5 Hz), 4.51 (2H, broad), 6.20-6.35 (1H, broad, Z/E forms), 6.38 (1H, d, J=3.6 Hz), 7.39 (1H, d, J=8.5 Hz), 7.48 (1H, broad, Z/E forms), 7.53 (1H, d, J=1.8 Hz), 7.68 (1H, broad), 8.00-8.8.15 (1H, broad, Z/E forms), 9.15-9.35 (1H, broad, Z/E forms). LCMS t_{R} 1.45 (min). MS (APCI), m/z 464.99 [M+H]⁺. Mₚ 65-67° C.

### 40. 6-Ethoxy-N-furan-2-ylmethyl-N′-(2-morpholin-4-ylmethyl-1H-benzoimidazol-5-yl)-[1,3,5]triazine-2,4-diamine

¹H-NMR (400 MHz, CDCl₃) δ_{H}: 1.42 (3H, broad), 2.60 (4H, m), 3.77 (4H, m), 3.82 (2H, s), 4.41 (2H, broad), 4.66 (2H, broad), 5.42 (1H, broad), 6.28 (1H, broad), 6.35 (1H, broad), 7.00 (1H, broad), 7.12 (1H, broad), 7.39 (1H, broad), 7.62 (1H, d, J=8.5 Hz), 8.05 (1H, broad), 9.39 (1H, broad, Z/E forms). LCMS t_{R} (min): 1.39. MS (APCI), m/z 451.07 [M+H]⁺. HPLC t_{R} (min): 8.58. Mₚ 115-117° C.

### 41. N-(1-Cyclohexyl-2-methyl-1H-benzoimidazol-5-yl)-6-ethoxy-N′-furan-2-ylmethyl-[1,3,5]triazine-2,4-diamine

¹H-NMR (400 MHz, DMSO-D₆) δ_{H}: 1.28 (3H, t, J=7.5 Hz), 1.23-2.20 (10H, m), 2.49 (3H, s), 4.20 (1H, m), 4.23 (2H, broad, Z/E forms), 4.49 (2H, broad), 6.15-6.35 (1H, broad, Z/E forms), 6.38 (1H, broad), 7.39 (1H, broad), 7.53 (2H, m), 7.68 (1H, broad, Z/E forms), 7.95 (1H, broad), 9.05-9.30 (1H, broad, Z/E forms). LCMS t_{R} (min): 1.55. MS (APCI), m/z 448.06 [M+H]⁺. HPLC t_{R} (min): 11.17. Mₚ 216-218° C.

### 42. 6-Ethoxy-N-furan-2-ylmethyl-N′-(2-trifluoromethyl-1H-benzoimidazol-5-yl)-[1,3,5]triazine-2,4-diamine

A mixture of 6-Ethoxy-4-chloro-N′-(2-trifluoromethyl-1H-benzoimidazol-5-yl)-[1,3,5]triazine-2-amine (200 mg, 0.56 mmol), furfurylamine (110 mg, 1.12 mmol), NEt₃ (110 mg, 1.12 mmol) and acetonitrile (15 mL) was stirred at room temperature for 1 hour and at 50° C. for 3 hours, cooled to room temperature, diluted with water. The formed solid was collected by filtration and recrystallized from ethanol. The mother liquid was concentrated at reduced pressure. The residue was recrystallized from ethyl acetate/hexane and diethyl ether/hexane to give the product (90 mg, 38%). ¹H-NMR (400 MHz, DMSO-D₆) δ_{H}: 1.28 (3H, t, J=7.5 Hz), 4.30 (2H, broad), 4.40-4.60 (2H, broad, Z/E forms), 6.25 (1H, broad), 6.38 (1H, broad), 7.52 (1H, s), 7.58 (2H, m), 7.74 (1H, broad), 8.15-8.30 (1H, broad, Z/E forms), 9.35-9.60 (1H, broad, Z/E forms), 13.60 (1H, broad). LCMS t_{R} (min): 1.67. MS (APCI), m/z 419.08 [M+H]⁺. HPLC t_{R} (min): 11.58. Mₚ 188-190° C.

### 43. N-(4-Bromo-phenyl)-6-ethoxy-N′-furan-2-ylmethyl-[1,3,5]triazine-2,4-diamine

¹H-NMR (400 MHz, DMSO-D₆) δ_{H}: 1.28 (3H, t, J=7.5 Hz), 4.30 (2H, broad), 4.49 (2H, d, J=7.5 Hz), 6.25 (1H, dd, J=3.6, 1.8 Hz), 6.39 (1H, d, J=3.6 Hz), 7.41 (2H, d, J=8.5 Hz), 7.55 (1H, d, J=1.8 Hz), 7.70 (2H, broad), 7.82 (1H, broad), 9.40-9.60 (1H, broad, Z/E forms). LCMS t_{R} 3.01 (min). MS (APCI), m/z 389.95; 391.85 [M+H]⁺. Mₚ 172-174° C.

### 44. 6-Ethoxy-N-(4-methoxy-phenyl)-N′-methyl-N′-(5-methyl-furan-2-ylmethyl)-[1,3,5]triazine-2,4-diamine

¹H-NMR (400 MHz, DMSO-D₆) δ_{H}: 1.30 (3H, t, J=7.5 Hz), 2.22 (3H, s), 3.07 (3H, s), 3.72 (3H, s), 4.32 (2H, q, J=7.5 Hz), 4.73 (2H, s), 5.98 (1H, broad), 6.18 (1H, broad), 6.88 (2H, d, J=8.5 Hz), 7.59 (2H, d, J=8.5 Hz), 9.24 (1H, broad). LCMS t_{R} (min): 2.04. MS (APCI), m/z 370.10 [M+H]⁺. HPLC t_{R} (min): 14.26. Mₚ 114-117° C.

### 45. 6-Ethoxy-N-(4-methoxy-phenyl)-N′-[1-(5-methyl-furan-2-yl)-ethyl]-[1,3,5]triazine-2,4-diamine

¹H-NMR (400 MHz, DMSO-D₆) δ_{H}: 1.29 (3H, t, J=7.5 Hz), 1.44 (3H, d, J=7.5 Hz), 2.23 (3H, s), 3.73 (3H, s), 4.30 (2H, q, J=7.5 Hz), 5.20 (1H, m), 5.95 (1H, broad), 6.07 (1H, broad), 6.84 (2H, d, J=8.5 Hz), 7.46-7.53 (1H, broad), 7.60 (2H, broad), 8.99-9.19 (1H, broad, Z/E forms). LCMS t_{R} (min): 1.94. MS (APCI), m/z 370.00 [M+H]⁺. HPLC t_{R} (min): 13.25. Mₚ 57-59° C.

### 46. N-(3-Dimethylamino-propyl)-6-ethoxy-N′-(4-methoxy-phenyl)-N-(5-methyl-furan-2-ylmethyl)-[1,3,5]triazine-2,4-diamine

¹H-NMR (400 MHz, DMSO-D₆) δ_{H}: 1.29 (3H, t, J=7.5 Hz), 1.67 (2H, m), 2.09 (6H, s), 2.19 (2H, t, J=7.5 Hz), 2.21 (3H, s), 3.50 (2H, t, broad, J=7.5 Hz), 3.71 (3H, s), 4.30 (2H, q, J=7.5 Hz), 4.72 (2H, broad), 5.98 (1H, broad), 6.16 (1H, broad), 6.84 (2H, d, J=8.5 Hz), 7.58 (2H, broad), 9.21 (1H, broad). LCMS t_{R} (min): 1.55. MS (APCI), m/z 441.10 [M+H]⁺. HPLC t_{R} (min): 10.99.

### 47. 6-Ethoxy-N-(4-methoxy-phenyl)-N′-(5-methyl-furan-2-ylmethyl)-N′-(3-morpholin-4-yl-propyl)-[1,3,5]triazine-2,4-diamine

¹H-NMR (400 MHz, DMSO-D₆) δ_{H}: 1.29 (3H, t, J=7.5 Hz), 1.69 (2H, m), 2.21 (3H, s), 2.29 (6H, m), 3.53 (6H, m), 3.72 (3H, s), 4.30 (2H, broad), 4.73 (2H, broad), 5.99 (1H, broad), 6.19 (1H, broad), 6.85 (2H, d, J=8.5 Hz), 7.59 (2H, d, J=8.5 Hz), 9.21 (1H, broad). LCMS t_{R} (min): 1.59. MS (APCI), m/z 483.11 [M+H]⁺. HPLC t_{R} (min): 11.07.

### 48/ N-Benzyl-6-ethoxy-N′-(4-methoxy-phenyl)-N-(5-methyl-furan-2-ylmethyl)-[1,3,5]triazine-2,4-diamine

¹H-NMR (400 MHz, DMSO-D₆) δ_{H}: 1.20-1.38 (3H, broad, Z/E forms), 2.20 (3H, s), 3.58-3.68 (3H, broad, Z/E forms), 4.21-4.40 (2H, broad, Z/E forms), 4.72 (2H, broad), 4.79 (2H, broad), 5.95 (1H, broad), 6.15 (1H, broad), 6.72-6.90 (2H, broad, Z/E forms), 7.20-7.30 (3H, m), 7.32 (2H, t, J=8.5 Hz), 7.41-7.67 (2H, broad, Z/E forms), 9.22-9.35 (1H, broad, Z/E forms). LCMS t_{R} (min): 2.23. MS (APCI), m/z 446.13 [M+H]⁺. HPLC t_{R} (min): 16.68. Mₚ 99-101° C.

### 49. N-Benzo[1,3]dioxol-5-ylmethyl-6-ethoxy-N′-(4-methoxy-phenyl)-N-(5-methyl-furan-2-ylmethyl)-[1,3,5]triazine-2,4-diamine

¹H-NMR (400 MHz, DMSO-D₆) δ_{H}: 1.20-1.37 (3H, broad, Z/E forms), 2.21 (3H, s), 3.21 (3H, s), 3.71 (2H, s), 4.23-4.38 (2H, broad, Z/E forms), 4.70 (2H, s), 5.97 (2H, s), 6.15 (1H, broad), 6.71 (1H, d, J=8.5 Hz), 6.80 (1H, broad), 6.85 (3H, m), 7.44-7.64 (2H, broad, Z/E forms), 9.23-9.34 (1H, broad, Z/E forms). LCMS t_{R} (min): 2.17. MS (APCI), m/z 490.12 [M+H]⁺. HPLC t_{R} (min): 16.07. Mₚ 88-90° C.

### 50. 6-Ethoxy-N-(4-methoxy-phenyl)-N′-(5-methyl-furan-2-ylmethyl)-N″-pyridin-2-ylmethyl-[1,3,5]triazine-2,4-diamine

¹H-NMR (400 MHz, DMSO-D₆) δ_{H}: 1.22-1.38 (3H, broad, Z/E forms), 2.18 (3H, s), 3.66-3.78 (3H, broad, Z/E forms), 4.12-4.40 (2H, broad, Z/E forms), 4.84 (4H, m), 5.94 (1H, broad), 6.16 (1H, broad), 6.69-6.90 (2H, broad, Z/E forms), 7.15 (1H, d, J=8.0 Hz), 7.24 (1H, d/d, J=8.0/5.0 Hz), 7.32-7.65 (2H, broad, Z/E forms), 7.70 (1H, t, J=8.0 Hz), 8.51 (1H, broad), 9.18-9.32 (1H, broad, Z/E forms). LCMS t_{R} (min): 1.74. MS (APCI), m/z 447.08 [M+H]⁺. HPLC t_{R} (min): 11.46.

### 51. 6-Ethoxy-N-furan-2-ylmethyl-N′-(4-methoxy-phenyl)-N-(5-methyl-furan-2-ylmethyl)-[1,3,5]triazine-2,4-diamine

¹H-NMR (400 MHz, DMSO-D₆) δ_{H}: 1.29 (3H, t, J=7.5 Hz), 2.20 (3H, s), 3.72 (3H, s), 4.32 (2H, q, J=7.5 Hz), 4.72 (2H, s), 4.78 (2H, s), 5.97 (1H, broad), 6.13 (1H, broad), 6.27 (1H, broad), 6.38 (1H, broad), 6.85 (2H, d, J=8.5 Hz), 7.57 (3H, broad), 9.31 (1H, broad). LCMS t_{R} (min): 2.14. MS (APCI), m/z 436.11 [M+H]⁺. HPLC t_{R} (min): 16.08.

### 52. 6-Ethoxy-N-furan-2-ylmethyl-N′-(4-methoxy-phenyl)-N-thiophen-2-ylmethyl-[1,3,5]triazine-2,4-diamine

¹H-NMR (400 MHz, DMSO-D₆) δ_{H}: 1.28 (3H, broad), 3.72 (3H, s), 4.36 (2H, broad), 4.77 (2H, s), 4.91 (2H, s), 6.31 (1H, broad), 6.39 (1H, broad), 6.85 (2H, d, J=8.5 Hz), 6.97 (1H, broad), 7.05 (1H, broad), 7.39 (1H, broad), 7.68 (3H, m), 9.34 (1H, broad). LCMS t_{R} (min): 2.11. MS (APCI), m/z 438.02 [M+H]⁺. HPLC t_{R} (min): 15.99. Mₚ 81-83° C.

### 53. 1-(3-{4-Ethoxy-6-[(furan-2-ylmethyl)-amino]-[1,3,5]triazin-2-ylamino}-phenyl)-pyrrolidin-2-one

¹H-NMR (400 MHz, DMSO-D₆) δ_{H}: 1.28 (3H, t, J=7.5 Hz), 2.05 (2H, m), 3.25 (2H, m), 3.70-3.88 (2H, broad, Z/E forms), 4.32 (2H, broad), 4.45-4.59 (2H, broad, Z/E forms), 6.24 (1H, broad), 6.38 (1H, broad), 7.24 (1H, t, J=8.5 Hz), 7.32-7.48 (1H, broad, Z/E forms), 7.54 (1H, s), 7.69-7.83 (1H, broad, Z/E forms), 7.88-8.12 (1H, broad, Z/E forms), 9.30-9.48 (1H, broad, Z/E forms). LCMS t_{R} (min): 1.69. MS (APCI), m/z 392.11 [M+H]⁺. HPLC t_{R} (min): 11.48. Mₚ 82-84° C.

### 54. 6-Ethoxy-N-furan-2-ylmethyl-N′-(3-pyrrolidin-1-yl-phenyl)-[1,3,5]triazine-2,4-diamine

¹H-NMR (400 MHz, DMSO-D₆) δ_{H}: 1.30 (3H, t, J=7.5 Hz), 1.92 (4H, broad), 3.18 (4H, broad), 4.31 (2H, q, J=7.5 Hz), 4.43-4.60 (2H, broad, Z/E forms), 6.18 (1H, d, J=8.5 Hz), 6.24 (1H, broad), 6.38 (1H, broad), 6.83-7.05 (1H, broad, Z/E forms), 7.02 (1H, d, J=8.5 Hz), 7.00-7.22 (1H, broad, Z/E forms), 7.53 (1H, s), 7.61-7.78 (1H, broad, Z/E forms), 9.00-9.19 (1H, broad, Z/E forms). LCMS t_{R} (min): 1.96. MS (APCI), m/z 381.15 [M+H]⁺. HPLC t_{R} (min): 12.76. Mₚ 107-109° C.

### 55. 6-Ethoxy-N-furan-2-ylmethyl-N′-(3-morpholin-4-ylmethyl-phenyl)-[1,3,5]triazine-2,4-diamine

A mixture of compound I-2 (382 mg, 1.5 mmol), 3-morpholin-4-ylmethyl-phenylamine (289 mg, 1.5 mmol), K₂CO₃ (417 mg, 3.0 mmol) and DMSO (0.5 mL) was stirred at 90° C. for 3 hours, cooled to room temperature and diluted with water. The formed solid was collected by filtration and purified by column chromatography (silica gel, methanol/ethyl acetate) and by preparative TLC (methanol/ethyl acetate) to give the product (159 mg, 26%).

¹H-NMR (400 MHz, DMSO-D₆) δ_{H}: 1.28 (3H, t, J=7.5 Hz), 2.33 (4H, m), 3.40 (2H, m), 3.58 (4H, m), 4.31 (2H, q, J=7.5 Hz), 4.52 (2H, broad), 6.26 (1H, broad), 6.38 (1H, broad), 6.91 (1H, d, J=8.5 Hz), 7.20 (1H, t, J=8.5 Hz), 7.51 (2H, m), 7.68 (1H, broad), 7.72-7.89 (1H, broad), 9.18-9.39 (1H, broad, Z/E forms). LCMS t_{R} (min): 1.42. MS (APCI), m/z 411.15 [M+H]⁺. HPLC t_{R} (min): 9.21. Mₚ 69-71° C.

### 56. 6-Ethoxy-N-furan-2-ylmethyl-N′-(3-trifluoromethoxy-phenyl)-[1,3,5]triazine-2,4-diamine

Yield 87 mg, 14%. ¹H-NMR (400 MHz, DMSO-D₆) δ_{H}: 1.25 (3H, t, J=7.5 Hz), 4.30 (2H, broad), 4.49 (2H, broad), 6.22 (1H, broad), 6.35 (1H, broad), 6.89 (1H, d, J=8.5 Hz), 7.35 (1H, t, J=8.5 Hz), 7.52 (1H, s), 7.58-7.73 (1H, broad, Z/E forms), 7.82-7.99 (1H, broad, Z/E forms), 7.88 (1H, broad), 9.52-9.74 (1H, broad, Z/E forms). LCMS t_{R} (min): 2.03. MS (APCI), m/z 396.03 [M+H]⁺. HPLC t_{R} (min): 15.17. Mₚ 128-130° C.

### 57. 6-Ethoxy-N-furan-2-ylmethyl-N′-(2-morpholin-4-ylmethyl-1H-benzoimidazol-5-yl)-[1,3,5]triazine-2,4-diamine

Compound 40 (30 mg. 0.07 mmol) was dissolved in a mixture of acetonitrile (3 mL) and water (3 mL). Then 1N CF₃COOH aqueous solution (0.21 mL, 0.21 mmol) was added to the obtained solution. The mixture was stirred for 30 minutes at room temperature. The solvent was removed in high vacuum at low temperature giving the TFA salt (34 mg, 90%). HPLC t_{R} (min): 8.63. Mₚ 60-62° C.

### 58. 6-Ethoxy-N-furan-2-ylmethyl-N′-(1-methyl-1,2,3,4-tetrahydro-quinolin-7-yl)-[1,3,5]triazine-2,4-diamine

¹H-NMR (400 MHz, CDCl₃) δ_{H}: 1.40 (3H, t, J=7.5 Hz), 1.99 (2H, m), 2.73 (2H, t, J=7.5 Hz), 2.90 (3H, s), 3.23 (2H, t, J=7.5 Hz), 4.40 (2H, broad), 4.68 (2H, d, J=7.5 Hz), 5.32 (1H, broad), 6.25 (1H, broad), 6.33 (1H, broad), 6.75 (1H, d, J=8.5 Hz), 6.80 (1H, broad), 6.90 (1H, d, J=8.5 Hz), 6.95 (1H, broad), 7.38 (1H, broad). LCMS t_{R} (min): 1.84. MS (APCI), m/z 381.15 [M+H]⁺. HPLC t_{R} (min): 11.47. Mₚ 76-78° C.

### 59. 6-Ethoxy-N-furan-2-ylmethyl-N′-(2-pyridin-3-yl-1H-benzoimidazol-5-yl)-[1,3,5]triazine-2,4-diamine

¹H-NMR (400 MHz, DMSO-D₆) δ_{H}: 1.31 (3H, t, J=7.5 Hz), 4.31 (2H, broad), 4.52 (2H, broad), 6.29 (1H, broad), 6.39 (1H, broad), 7.51 (3H, m), 7.56 (1H, s), 7.72 (1H, broad), 8.17 (1H, broad), 8.47 (1H, d, J=8.0 Hz), 8.65 (1H, d, J=5.0 Hz), 9.32 (1H, s), 9.44 (1H, broad), 12.89 (1H, broad). LCMS t_{R} (min): 1.45. MS (APCI), m/z 429.03 [M+H]⁺. HPLC t_{R} (min): 9.00. Mₚ 127-129° C.

### 60. N-[2-(4-Dimethylamino-phenyl)-1H-benzoimidazol-5-yl]-6-ethoxy-N′-furan-2-ylmethyl-[1,3,5]triazine-2,4-diamine

¹H-NMR (400 MHz, DMSO-D₆) δ_{H}: 1.30 (3H, broad), 2.99 (6H, s), 4.32 (2H, broad), 4.52 (2H, broad), 6.28 (1H, broad), 6.38 (1H, broad), 6.82 (2H, d, J=8.5 Hz), 7.39 (2H, broad), 7.54 (1H, s), 7.67 (1H, broad), 7.95 (1H, broad), 7.98 (2H, d, J=8.5 Hz), 9.17-9.34 (1H, broad, Z/E forms), 12.00 (1H, broad). LCMS t_{R} (min): 1.56. MS (APCI), m/z 471.16 [M+H]⁺. HPLC t_{R} (min): 10.73. Mₚ 153-155° C.

### 61. 6-Ethoxy-N-[2-(4-fluoro-phenyl)-benzooxazol-5-yl]-N′-furan-2-ylmethyl-[1,3,5]triazine-2,4-diamine

¹H-NMR (400 MHz, DMSO-D₆) δ_{H}: 1.30 (3H, t, J=7.5 Hz), 4.33 (2H, broad), 4.52 (2H, broad), 6.23-6.33 (1H, broad, Z/E forms), 6.38 (1H, broad), 7.44 (2H, m), 7.57 (1H, s), 7.66 (2H, m), 7.85 (1H, broad), 8.24 (2H, broad), 8.25-8.42 (1H, broad, Z/E forms), 9.45-9.59 (1H, broad, Z/E forms). LCMS t_{R} (min): 2.02. MS (APCI), m/z 447.10 [M+H]⁺. HPLC t_{R} (min): 14.50. Mₚ 105-107° C.

### 62. 6-Ethoxy-N-furan-2-ylmethyl-N′-(2-pyridin-3-yl-benzooxazol-5-yl)-[1,3,5]triazine-2,4-diamine

¹H-NMR (400 MHz, DMSO-D₆) δ_{H}: 1.30 (3H, t, J=7.5 Hz), 4.32 (2H, broad), 4.52 (2H, broad), 6.22-6.35 (1H, broad, Z/E forms), 6.39 (1H, broad), 7.53 (1H, s), 7.65 (1H, broad), 7.70 (2H, broad), 7.82-7.98 (1H, broad, Z/E forms), 8.30-8.48 (1H, broad, Z/E forms), 8.52 (1H, d, J=8.0 Hz), 8.80 (1H, d, J=5.0 Hz), 9.33 (1H, d, J=1.5 Hz), 9.50-9.65 (1H, broad, Z/E forms). LCMS t_{R} (min): 1.70. MS (APCI), m/z 430.04 [M+H]⁺. HPLC t_{R} (min): 11.45. Mₚ 198-200° C.

### 63. 6-{4-Ethoxy-6-[(furan-2-ylmethyl)-amino]-[1,3,5]triazin-2-ylamino}-3H-benzothiazol-2-one

¹H-NMR (400 MHz, DMSO-D₆) δ_{H}: 1.30 (3H, broad), 4.30 (2H, broad), 4.49 (2H, broad), 6.23 (1H, broad), 6.39 (1H, broad), 7.01 (1H, d, J=8.5 Hz), 7.45 (1H, d, J=8.5 Hz), 7.55 (1H, s), 7.78 (1H, broad), 7.91-8.19 (1H, broad, Z/E forms), 9.28-9.42 (1H, broad, Z/E forms), 11.60 (1H, broad). LCMS t_{R} (min): 1.62. MS (APCI), m/z 385.05 [M+H]⁺. HPLC t_{R} (min): 10.64. Mₚ 298-270° C. (decomp.).

### 64. N-(6-{4-Ethoxy-6-[(furan-2-ylmethyl)-amino]-[1,3,5]triazin-2-ylamino}-benzothiazol-2-yl)-acetamide

¹H-NMR (400 MHz, DMSO-D₆) δ_{H}: 1.29 (3H, t, J=7.5 Hz), 2.20 (3H, s), 4.32 (2H, broad), 4.52 (2H, broad), 6.27 (1H, broad), 6.39 (1H, broad), 7.55 (1H, s), 7.61 (2H, m), 7.81 (1H, broad), 8.35-8.65 (1H, broad, Z/E forms), 9.40-9.58 (1H, broad, Z/E forms), 12.12 (1H, broad, Z/E forms). LCMS t_{R} (min): 1.65. MS (APCI), m/z 426.07 [M+H]⁺. HPLC t_{R} (min): 10.77. Mₚ 257-259° C.

### 65. 6-Ethoxy-N-furan-2-ylmethyl-N′-(1H-indazol-6-yl)-[1,3,5]triazine-2,4-diamine

¹H-NMR (400 MHz, DMSO-D₆) δ_{H}: 1.31 (3H, t, J=7.5 Hz), 4.34 (2H, broad), 4.53 (2H, broad), 6.28 (1H, broad), 6.38 (1H, broad), 7.37 (1H, broad), 7.54 (1H, s), 7.60 (1H, d, J=8.5 Hz), 7.67-7.83 (1H, broad, Z/E forms), 7.92 (1H, s), 8.04-8.20 (1H, broad, Z/E forms), 9.35-9.54 (1H, broad, Z/E forms), 12.77 (1H, broad). LCMS t_{R} (min): 1.62. MS (APCI), m/z 352.09 [M+H]⁺. HPLC t_{R} (min): 10.68. Mₚ 201-203° C.

### 66. 6-Ethoxy-N-(furan-2-ylmethyl)-N′-(1H-indazol-6-yl)-1,3,5-triazine-2,4-diamine

¹H-NMR (400 MHz, DMSO-D₆) δ_{H}: 1.29 (3H, t, J=7.5 Hz), 4.32 (2H, broad), 4.52 (2H, broad), 6.27 (1H, broad), 6.38 (1H, broad), 7.55 (1H, s), 7.73 (1H, d, J=8.5 Hz), 7.91 (1H, broad), 7.95 (1H, d, J=8.5 Hz), 8.58-8.73 (1H, broad, Z/E forms), 9.19 (1H, s), 9.57-9.71 (1H, broad, Z/E forms). LCMS t_{R} (min): 1.71. MS (APCI), m/z 369.04 [M+H]⁺. HPLC t_{R} (min): 11.82. Mₚ 191-193° C.

### 67. N-(2-Cyclohexyl-1H-benzoimidazol-5-yl)-6-ethoxy-N′-furan-2-ylmethyl-[1,3,5]triazine-2,4-diamine

¹H-NMR (400 MHz, DMSO-D₆) δ_{H}: 1.30 (3H, t, J=7.5 Hz), 1.36 (1H, m), 1.41 (2H, m), 1.60 (2H, m), 1.70 (1H, m), 1.80 (2H, m), 2.01 (2H, m), 2.80 (1H, m), 4.30 (2H, broad), 4.51 (2H, broad), 6.26 (1H, broad), 6.39 (1H, broad), 7.33 (2H, broad), 7.53 (1H, s), 7.68 (1H, broad), 7.95 (1H, broad), 9.12-9.34 (1H, broad, Z/E forms), 11.89 (1H, broad). LCMS t_{R} (min): 1.54. MS (APCI), m/z 434.25 [M+H]⁺. HPLC t_{R} (min): 10.44. Mₚ 165-167° C.

### 68. 6-Ethoxy-N-furan-2-ylmethyl-N′-[3-(4H-[1,2,4]triazol-3-yl)-phenyl]-[1,3,5]triazine-2,4-diamine

¹H-NMR (400 MHz, DMSO-D₆) δ_{H}: 1.30 (3H, t, J=7.5 Hz), 4.32 (2H, broad), 4.45-4.61 (2H, broad, Z/E forms), 6.25 (1H, broad), 6.37 (1H, broad), 7.35 (1H, broad), 7.52 (1H, s), 7.62 (1H, broad), 7.79 (2H, broad), 7.95-8.40 (1H, broad, Z/E forms), 8.55 (1H, broad), 9.38-9.60 (1H, broad, Z/E forms), 13.90-14.40 (1H, broad, Z/E forms). LCMS t_{R} (min): 1.57. MS (APCI), m/z 379.08 [M+H]⁺. HPLC t_{R} (min): 9.89. Mₚ 231-233° C.

### 69. 6-Ethoxy-N-furan-2-ylmethyl-N′-[3-(5-methyl-tetrazol-1-yl)-phenyl]-[1,3,5]triazine-2,4-diamine

¹H-NMR (400 MHz, DMSO-D₆) δ_{H}: 1.28 (3H, t, J=7.5 Hz), 2.55 (3H, s), 4.30 (2H, broad), 4.48 (2H, broad), 6.12-6.25 (1H, broad, Z/E forms), 6.29-6.38 (1H, broad, Z/E forms), 7.22 (1H, broad), 7.42-7.49 (1H, broad, Z/E forms), 7.52 (1H, d, J=8.5 Hz), 7.87 (2H, broad), 8.08-8.22 (1H, broad, Z/E forms), 9.63-9.82 (1H, broad, Z/E forms). LCMS t_{R} (min): 1.66. MS (APCI), m/z 393.93 [M+H]⁺. HPLC t_{R} (min): 11.78. Mₚ 146-148° C.

### 70. 6-Ethoxy-N-furan-2-ylmethyl-N′-(3-pyrrol-1-yl-phenyl)-[1,3,5]triazine-2,4-diamine

¹H-NMR (400 MHz, DMSO-D₆) δ_{H}: 1.30 (3H, t, J=7.5 Hz), 4.32 (2H, broad), 4.51 (2H, broad), 6.25 (3H, m), 6.38 (1H, broad), 7.12 (1H, d, J=8.5 Hz), 7.18 (1H, broad), 7.27 (1H, broad), 7.34 (1H, t, J=8.5 Hz), 7.53 (1H, s), 7.59 (1H, broad), 7.84 (1H, broad), 7.98-8.06 (1H, broad, Z/E forms), 9.38-9.57 (1H, broad, Z/E forms). LCMS t_{R} (min): 1.97. MS (APCI), m/z 377.06 [M+H]⁺. HPLC t_{R} (min): 14.41. Mₚ 144-146° C.

### 71. 6-Ethoxy-N-furan-2-ylmethyl-N′-[3-(2-methyl-imidazol-1-ylmethyl)-phenyl]-[1,3,5]triazine-2,4-diamine

¹H-NMR (400 MHz, DMSO-D₆) δ_{H}: 1.29 (3H, t, J=7.5 Hz), 2.22 (3H, s), 4.29 (2H, broad), 4.49 (2H, s), 5.09 (2H, broad), 6.25 (1H, broad), 6.48 (1H, broad), 6.73 (1H, d, J=8.5 Hz), 6.79 (1H, s), 7.08 (1H, broad), 7.24 (1H, broad), 7.53 (1H, s), 7.58 (1H, broad), 7.65 (1H, d, J=8.5 Hz), 7.67-7.82 (1H, broad, Z/E forms), 9.32-9.49 (1H, broad, Z/E forms). LCMS t_{R} (min): 1.44. MS (APCI), m/z 406.06 [M+H]⁺. HPLC t_{R} (min): 9.18. Mₚ 193-195° C.

### 72. 6-Ethoxy-N-furan-2-ylmethyl-N′-[3-(4-methyl-piperazin-1-ylmethyl)-phenyl]-[1,3,5]triazine-2,4-diamine

¹H-NMR (400 MHz, DMSO-D₆) δ_{H}: 1.30 (3H, t, J=7.5 Hz), 2.13 (3H, s), 2.28 (4H, broad), 2.34 (4H, broad), 3.40 (2H, broad), 4.31 (2H, broad), 4.51 (2H, broad), 6.25 (1H, broad), 6.37 (1H, broad), 6.90 (1H, d, J=8.5 Hz), 7.19 (1H, t, J=8.5 Hz), 7.50 (1H, broad), 7.53 (1H, s), 7.58-7.72 (1H, broad, Z/E forms), 7.72-7.87 (1H, broad, Z/E forms), 9.21-9.39 (1H, broad, Z/E forms). LCMS t_{R} (min): 1.3. MS (APCI), m/z 423.96 [M+H]⁺. HPLC t_{R} (min): 815. Mₚ 83-85° C.

### 73. 6-Ethoxy-N-furan-2-ylmethyl-N′-(2-trifluoromethyl-phenyl)-[1,3,5]triazine-2,4-diamine

¹H-NMR (400 MHz, DMSO-D₆) δ_{H}: 1.22 (3H, broad), 4.22 (2H, broad), 4.30-4.47 (2H, broad, Z/E forms), 6.00-6.13 (1H, broad, Z/E forms), 6.35 (1H, broad), 7.42 (1H, t, J=8.5 Hz), 7.52 (1H, s), 7.63 (2H, m), 7.72 (2H, m), 8.52-8.72 (1H, broad, Z/E forms).

LCMS t_{R} (min): 1.93. MS (APCI), m/z 380.04 [M+H]⁺. HPLC t_{R} (min): 14.21. Mₚ 70-72° C.

### 74. 6-Ethoxy-N-furan-2-ylmethyl-N′-(3-pyrazol-1-yl-phenyl)-[1,3,5]triazine-2,4-diamine

¹H-NMR (400 MHz, DMSO-D₆) δ_{H}: 1.30 (3H, t, J=7.5 Hz), 4.34 (2H, broad), 4.48-4.60 (2H, broad, Z/E forms), 6.25 (1H, s), 6.38 (1H, broad), 6.51 (1H, broad), 7.35 (1H, t, J=8.5 Hz), 7.40 (1H, broad), 7.53 (1H, s), 7.54-7.69 (1H, broad, Z/E forms), 7.72 (1H, broad), 7.88 (1H, broad), 8.28 (1H, broad), 8.32-8.50 (1H, broad, Z/E forms), 9.45-9.65 (1H, broad, Z/E forms). LCMS t_{R} (min): 1.81. MS (APCI), m/z 378.16 [M+H]⁺. HPLC t_{R} (min): 12.70. Mₚ 138-140° C.

### 75. 6-Ethoxy-N-furan-2-ylmethyl-N′-(3-[1,2,4]triazol-1-ylmethyl-phenyl)-[1,3,5]triazine-2,4-diamine

¹H-NMR (400 MHz, DMSO-D₆) δ_{H}: 1.28 (3H, t, J=7.5 Hz), 4.30 (2H, broad), 4.50 (2H, d, J=7.5 Hz), 5.38 (2H, s), 6.25 (1H, broad), 6.40 (1H, broad), 6.88 (1H, broad), 7.25 (1H, t, J=8.5 Hz), 7.55 (1H, s), 7.59-7.68 (1H, broad, Z/E forms), 7.70 (1H, broad), 7.72-7.82 (1H, broad, Z/E forms), 7.98 (1H, broad), 8.52-8.65 (1H, broad, Z/E forms), 9.31-9.50 (1H, broad, Z/E forms). LCMS t_{R} (min): 1.59. MS (APCI), m/z 393.14 [M+H]⁺. HPLC t_{R} (min): 10.22. Mₚ 141-143° C.

### 76. N-(3-Benzothiazol-2-yl-phenyl)-6-ethoxy-N′-furan-2-ylmethyl-[1,3,5]triazine-2,4-diamine

¹H-NMR (400 MHz, CDCl₃) δ_{H}: 1.41 (3H, broad), 4.46 (2H, broad), 4.75 (2H, broad), 5.64 (1H, broad), 6.29 (1H, broad), 6.32 (1H, broad), 7.21 (1H, broad), 7.38 (1H, broad), 7.42 (1H, t, J=8.5 Hz), 7.44 (1H, t, J=8.5 Hz), 7.50 (1H, t, J=8.5 Hz), 7.57-7.72 (1H, broad, Z/E forms), 7.79 (1H, d, J=8.5 Hz), 7.91 (1H, d, J=8.5 Hz), 8.05 (1H, broad), 8.35-8.75 (1H, broad, Z/E forms). LCMS t_{R} (min): 2.15. MS (APCI), m/z 445.09 [M+H]⁺. HPLC t_{R} (min): 15.38. Mₚ 42-44° C.

77. Pyridine-2-carboxylic acid (3-{4-ethoxy-6-[(furan-2-ylmethyl)-amino]-[1,3,5]triazin-2-ylamino}-phenyl)-amide

¹H-NMR (400 MHz, DMSO-D₆) δ_{H}: 1.29 (3H, t, J=7.5 Hz), 4.31 (2H, broad), 4.44-4.63 (2H, broad, Z/E forms), 6.25 (1H, broad), 6.37 (1H, broad), 7.24 (1H, t, J=8.5 Hz), 7.42 (1H, broad, Z/E forms), 7.52 (2H, broad), 7.67 (1H, broad), 7.78 (1H, broad), 8.05 (1H, t, J=8.5 Hz), 8.10-8.40 (1H, broad, Z/E forms), 8.25 (1H, broad), 8.72 (1H, broad), 9.33-9.49 (1H, broad, Z/E forms), 10.29-10.40 (1H, broad, Z/E forms). LCMS t_{R} (min): 1.80. MS (APCI), m/z 432.07 [M+H]⁺. HPLC t_{R} (min): 12.64. Mₚ 181-183° C.

### 78. 6-Ethoxy-N-[3-(5-ethyl-benzooxazol-2-yl)-phenyl]-N′-furan-2-ylmethyl-[1,3,5]triazine-2,4-diamine

¹H-NMR (400 MHz, DMSO-D₆) δ_{H}: 1.25 (3H, t, J=7.5 Hz), 1.33 (3H, broad), 2.75 (2H, q, J=7.5 Hz), 4.38 (2H, broad), 4.46-4.69 (2H, broad, Z/E forms), 6.23-6.32 (1H, broad, Z/E forms), 6.39 (1H, broad), 7.27 (1H, broad), 7.49 (1H, t, J=8.5 Hz), 7.52 (1H, s), 7.53-7.70 (2H, m), 7.80 (1H, d, J=8.5 Hz), 7.83 (1H, broad), 7.88-8.10 (1H, broad, Z/E forms), 8.53-9.00 (1H, broad, Z/E forms), 9.59-9.79 (1H, broad, Z/E forms). LCMS t_{R} (min): 2.24. MS (APCI), m/z 457.12 [M+H]⁺. HPLC t_{R} (min): 16.28. Mₚ 59-61° C.

### 79. 6-Ethoxy-N-furan-2-ylmethyl-N′-(3-quinoxalin-2-yl-phenyl)-[1,3,5]triazine-2,4-diamine

¹H-NMR (400 MHz, CDCl₃) δ_{H}: 1.34-1.62 (3H, broad, Z/E forms), 4.46 (2H, broad), 4.73 (2H, broad), 5.51 (1H, broad), 6.28 (1H, broad), 6.32 (1H, broad), 7.14 (1H, broad), 7.38 (1H, s), 7.52 (1H, t, J=8.5 Hz), 7.75 (3H, broad), 7.89 (1H, d, J=8.5 Hz), 8.13 (2H, broad), 8.43-8.75 (1H, broad, Z/E forms), 9.35 (1H, s). LCMS t_{R} (min): 2.00. MS (APCI), m/z 440.12 [M+H]⁺. HPLC t_{R} (min): 13.97.

### 80. N-(3-{4-Ethoxy-6-[(furan-2-ylmethyl)-amino]-[1,3,5]triazin-2-ylamino}-phenyl)-2-morpholin-4-yl-acetamide

Yield 242 mg, 36%. ¹H-NMR (400 MHz, DMSO-D₆) δ_{H}: 1.30 (3H, t, J=7.5 Hz), 2.55 (4H, m), 3.15 (2H, broad), 3.64 (4H, m), 4.32 (2H, broad), 4.52 (2H, broad), 6.25 (1H, broad), 6.37 (1H, broad), 7.18 (1H, d, J=8.5 Hz), 7.22 (1H, broad), 7.30-7.48 (1H, broad, Z/E forms), 7.52 (1H, s), 7.60-7.79 (1H, broad, Z/E forms), 7.80-8.20 (1H, broad, Z/E forms), 9.22-9.42 (1H, broad, Z/E forms), 9.58 (1H, broad). LCMS t_{R} (min): 1.42. MS (APCI), m/z 454.13 [M+H]⁺. HPLC t_{R} (min): 8.82. Mₚ 163-165° C.

### 81. 6-Ethoxy-N-furan-2-ylmethyl-N′-[3-(2-pyridin-2-yl-ethyl)-phenyl]-[1,3,5]triazine-2,4-diamine

¹H-NMR (400 MHz, DMSO-D₆) δ_{H}: 1.29 (3H, t, J=7.5 Hz), 2.95 (2H, broad), 3.02 (2H, broad), 4.30 (2H, broad), 4.50 (2H, broad), 6.24 (1H, broad), 6.37 (1H, broad), 6.82 (1H, d, J=8.5 Hz), 7.14 (1H, t, J=8.5 Hz), 7.21 (2H, broad), 7.52 (2H, broad), 7.68 (3H, broad), 8.50 (1H, d, J=5.0 Hz), 9.17-9.34 (1H, broad, Z/E forms). LCMS t_{R} (min): 1.49. MS (APCI), m/z 417.09 [M+H]⁺. HPLC t_{R} (min): 9.49. Mₚ 102-104° C.

### 82. 6-Ethoxy-N-furan-2-ylmethyl-N′-[3-(4-methyl-4H-[1,2,4]triazol-3-yl)-phenyl]-[1,3,5]triazine-2,4-diamine

¹H-NMR (400 MHz, DMSO-D₆) δ_{H}: 1.27 (3H, t, J=7.5 Hz), 3.72 (3H, s), 4.30 (2H, broad), 4.49 (2H, broad), 6.21 (1H, broad), 6.38 (1H, broad), 7.30 (1H, d, J=8.5 Hz), 7.41 (1H, t, J=8.5 Hz), 7.52 (1H, broad), 7.84 (1H, broad), 7.92 (1H, broad), 8.07 (1H, broad), 8.51 (1H, s), 9.49-9.67 (1H, broad, Z/E forms). LCMS t_{R} (min): 1.53. MS (APCI), m/z 392.76 [M+H]⁺. HPLC t_{R} (min): 9.57. Mₚ 120-122° C.

### 83. 6-Ethoxy-N-furan-2-ylmethyl-N′-[3-(5-methyl-1H-benzoimidazol-2-yl)-phenyl]-[1,3,5]triazine-2,4-diamine

¹H-NMR (400 MHz, DMSO-D₆) δ_{H}: 1.30 (3H, t, J=7.5 Hz), 2.42 (3H, s), 4.35 (2H, broad), 4.48-4.68 (2H, broad, Z/E forms), 6.28 (1H, broad), 6.37 (1H, broad), 7.02 (1H, broad), 7.27-7.38 (1H, broad, Z/E forms), 7.42 (1H, t, J=8.5 Hz), 7.53 (2H, broad), 7.72 (1H, d, J=8.5 Hz), 7.78 (2H, broad), 8.43-8.78 (1H, broad, Z/E forms), 9.41-9.58 (1H, broad, Z/E forms), 12.58 (1H, broad). LCMS t_{R} (min): 1.59. MS (APCI), m/z 442.18 [M+H]⁺. HPLC t_{R} (min): 10.97. Mₚ 82-84° C.

### 84. N-{3-[2-(1H-Benzoimidazol-2-yl)-ethyl]-phenyl}-6-ethoxy-N′-furan-2-ylmethyl-[1,3,5]triazine-2,4-diamine

¹H-NMR (400 MHz, DMSO-D₆) δ_{H}: 1.30 (3H, t, J=7.5 Hz), 3.10 (4H, m), 4.20-4.35 (2H, broad, Z/E forms), 4.51 (2H, broad), 6.23 (1H, broad), 6.37 (1H, broad), 6.88 (1H, d, J=8.5 Hz), 7.11 (2H, broad), 7.17 (1H, t, J=8.5 Hz), 7.47 (2H, broad), 7.54 (2H, broad), 7.69 (1H, s), 7.78 (1H, broad), 9.21-9.38 (1H, broad, Z/E forms), 12.18 (1H, broad). LCMS t_{R} (min): 1.54. MS (APCI), m/z 456.16 [M+H]⁺. HPLC t_{R} (min): 10.07. Mₚ 55-57° C.

### 85. N-(3-Azepan-1-ylmethyl-phenyl)-6-ethoxy-N′-furan-2-ylmethyl-[1,3,5]triazine-2,4-diamine

¹H-NMR (400 MHz, DMSO-D₆) δ_{H}: 1.30 (3H, t, J=7.5 Hz), 1.59 (8H, m), 2.60 (4H, m), 3.58 (2H, broad), 4.30 (2H, broad), 4.51 (2H, broad), 6.25 (1H, broad), 6.40 (1H, broad), 6.92 (1H, d, J=8.5 Hz), 7.20 (1H, t, J=8.5 Hz), 7.50 (1H, broad), 7.52 (1H, s), 7.67 (1H, broad), 7.71-7.84 (1H, broad, Z/E forms), 9.20-9.40 (1H, broad, Z/E forms). LCMS t_{R} (min): 1.52. MS (APCI), m/z 423.21 [M+H]⁺. HPLC t_{R} (min): 10.02. Mₚ 70-73° C.

### 86. 6-Ethoxy-N-furan-2-ylmethyl-N′-[3-(2-morpholin-4-yl-ethoxy)-phenyl]-[1,3,5]triazine-2,4-diamine

¹H-NMR (400 MHz, DMSO-D₆) δ_{H}: 1.29 (3H, t, J=7.5 Hz), 2.77 (6H, m), 3.60 (4H, m), 4.10 (2H, broad), 4.31 (2H, broad), 4.51 (2H, broad), 6.27 (1H, broad), 6.37 (1H, broad), 6.57 (1H, d, J=8.5 Hz), 7.15 (1H, t, J=8.5 Hz), 7.21-7.35 (1H, broad, Z/E forms), 7.41-7.55 (1H, broad, Z/E forms), 7.55 (1H, s), 7.80 (1H, broad), 9.22-9.40 (1H, broad, Z/E forms). LCMS t_{R} (min): 1.44. MS (APCI), m/z 441.15 [M+H]⁺. HPLC t_{R} (min): 9.18. Mₚ 70-72° C.

### 87. 6-Ethoxy-N-furan-2-ylmethyl-N′-[3-(2-pyridin-4-yl-ethyl)-phenyl]-[1,3,5]triazine-2,4-diamine

¹H-NMR (400 MHz, DMSO-D₆) δ_{H}: 1.29 (3H, t, J=7.5 Hz), 2.89 (4H, broad), 4.31 (2H, broad), 4.50 (2H, broad), 6.24 (1H, broad), 6.38 (1H, broad), 6.83 (1H, d, J=8.5 Hz), 6.86-7.16 (1H, two triplets, J=8.5 Hz, Z/E forms), 7.19 (1H, broad, Z/E forms), 7.22 (2H, d, J=5.0 Hz), 7.42-7.55 (1H, broad, Z/E forms), 7.50-7.67 (1H, broad, Z/E forms), 7.70-7.82 (1H, broad, Z/E forms), 8.42 (2H, d, J=5.0 Hz), 9.19-9.38 (1H, broad, Z/E forms). LCMS t_{R} (min): 1.50. MS (APCI), m/z 417.07 [M+H]⁺. HPLC t_{R} (min): 9.43. Mₚ 129-131° C.

### 88. 6-Ethoxy-N-furan-2-ylmethyl-N′-[3-(2H-tetrazol-5-yl)-phenyl]-[1,3,5]triazine-2,4-diamine

¹H-NMR (400 MHz, DMSO-D₆) δ_{H}: 1.30 (3H, broad triplet, J=7.5 Hz), 4.33 (2H, broad), 4.52 (2H, broad), 6.25 (1H, broad), 6.31-6.40 (1H, broad, Z/E forms), 7.50 (2H, m), 7.60 (1H, d, J=8.5 Hz), 7.75-7.90 (1H, broad, Z/E forms), 7.82-7.97 (1H, broad, Z/E forms), 8.43-8.62 (1H, broad, Z/E forms), 9.58-9.72 (1H, broad, Z/E forms), 16.60 (1H, broad). LCMS t_{R} (min): 1.65. MS (APCI), m/z 380.02 [M+H]⁺. HPLC t_{R} (min): 10.70. M_{P} 137-139° C.

### 89. N-(3-{4-Ethoxy-6-[(furan-2-ylmethyl)-amino]-[1,3,5]triazin-2-ylamino}-phenyl)-2-[4-(2-methoxy-phenyl)-piperazin-1-yl]-acetamide

¹H-NMR (400 MHz, DMSO-D₆) δ_{H}: 1.27 (3H, t, J=7.5 Hz), 2.70 (4H, m), 3.02 (4H, m), 3.19 (2H, s), 3.78 (3H, s), 4.30 (2H, broad), 4.41-4.60 (2H, broad, Z/E forms), 6.22 (1H, broad), 6.34 (1H, broad), 6.91 (4H, m), 7.19 (1H, t, J=8.5 Hz), 7.25 (1H, broad), 7.38 (1H, broad), 7.52 (1H, s), 7.62-7.80 (1H, broad, Z/E forms), 7.80-8.18 (1H, broad, Z/E forms), 9.27-9.43 (1H, broad, Z/E forms), 9.57 (1H, broad). LCMS t_{R} (min): 1.58. MS (APCI), m/z 559.36 [M+H]⁺. HPLC t_{R} (min): 10.67. Mₚ 96-98° C.

### 90. N-{3-[2-(4-Benzyl-piperazin-1-yl)-pyrimidin-4-yl]-phenyl}-6-ethoxy-N′-furan-2-ylmethyl-[1,3,5]triazine-2,4-diamine

¹H-NMR (400 MHz, DMSO-D₆) δ_{H}: 1.28 (3H, t, J=7.5 Hz), 2.48 (4H, m), 3.54 (2H, s), 3.87 (4H, m, broad), 4.30 (2H, broad), 4.51 (2H, broad), 6.23 (1H, broad), 6.37 (1H, broad), 7.00-7.12 (1H, broad, Z/E forms), 7.28 (1H, broad), 7.34 (4H, broad), 7.38 (1H, t, J=8.5 Hz), 7.53 (1H, s), 7.69 (1H, d, J=8.5 Hz), 7.73-7.85 (1H, broad, Z/E forms), 7.88-8.00 (1H, broad, Z/E forms), 8.39 (1H, broad), 8.47 (1H, broad), 9.43-9.58 (1H, broad, Z/E forms). LCMS t_{R} (min): 1.66. MS (APCI), m/z 564.29 [M+H]⁺. HPLC t_{R} (min): 11.30. Mₚ 100-102° C.

### 91. 6-Ethoxy-N-furan-2-ylmethyl-N′-[3-(4H-[1,2,4]triazol-3-yl)-phenyl]-[1,3,5]triazine-2,4-diamine

¹H-NMR (400 MHz, DMSO-D₆) δ_{H}: 1.30 (3H, t, J=7.5 Hz), 4.32 (2H, broad), 4.45-4.61 (2H, broad, Z/E forms), 6.25 (1H, broad), 6.37 (1H, broad), 7.35 (1H, broad), 7.52 (1H, s), 7.62 (1H, broad), 7.79 (2H, broad), 7.95-8.40 (1H, broad, Z/E forms), 8.55 (1H, broad), 9.38-9.60 (1H, broad, Z/E forms), 13.90-14.40 (1H, broad, Z/E forms). LCMS t_{R} (min): 1.57. MS (APCI), m/z 379.08 [M+H]⁺. HPLC t_{R} (min): 9.89. Mₚ 231-233° C.

### 92. 6-Ethoxy-N-furan-2-ylmethyl-N′-(3-morpholin-4-yl-phenyl)-[1,3,5]triazine-2,4-diamine

¹H-NMR (400 MHz, DMSO-D₆) δ_{H}: 1.29 (3H, t, J=7.5 Hz), 3.05 (4H, m), 3.72 (4H, m), 4.31 (2H, d, J=7.5 Hz), 4.48-4.60 (2H, broad), 6.24 (1H, broad), 6.38 (1H, broad), 6.59 (1H, d, J=8.5 Hz), 7.06-7.28 (1H, broad, Z/E forms), 7.11 (1H, broad), 7.37-7.52 (1H, broad, Z/E forms), 7.56 (1H, s), 7.80 (1H, broad), 9.11-9.32 (1H, broad, Z/E forms). LCMS t_{R} (min): 1.75. MS (APCI), m/z 397.15 [M+H]⁺. HPLC t_{R} (min): 11.32. Mₚ 92-95° C.

### 93. 6-Ethoxy-N-furan-2-ylmethyl-N′-(4-trifluoromethyl-phenyl)-[1,3,5]triazine-2,4-diamine

¹H-NMR (400 MHz, DMSO-D₆) δ_{H}: 1.30 (3H, t, J=7.5 Hz), 4.32 (2H, broad), 4.51 (2H, broad), 6.28 (1H, broad), 6.39 (1H, broad), 7.55 (1H, s), 7.60 (2H, broad), 7.95 (3H, broad), 9.70-9.84 (1H, broad, Z/E forms). LCMS t_{R} (min): 2.01. MS (APCI), m/z 380.06 [M+H]⁺. HPLC t_{R} (min): 15.19. Mₚ 139-142° C.

### 94. N-(3-Bromo-phenyl)-6-ethoxy-N′-furan-2-ylmethyl-[1,3,5]triazine-2,4-diamine

¹H-NMR (400 MHz, DMSO-D₆) δ_{H}: 1.30 (3H, t, J=7.5 Hz), 4.32 (2H, broad), 4.50 (2H, broad), 6.27 (1H, broad), 6.37 (1H, broad), 7.12 (1H, d, J=8.5 Hz), 7.22 (1H, t, J=8.5 Hz), 7.53 (1H, s), 7.61-7.78 (1H, broad, Z/E forms), 7.91 (1H, broad), 8.02-8.18 (1H, broad, Z/E forms), 9.44-9.62 (1H, broad, Z/E forms). LCMS t_{R} (min): 1.95. MS (APCI), m/z 389.91, 391.89 [M+H]⁺. HPLC t_{R} (min): 14.66. Mₚ 172-174° C.

### 96. 6-{4-Ethoxy-6-[(5-methyl-furan-2-ylmethyl)-amino]-[1,3,5]triazin-2-ylamino}-1,4-dihydro-quinoxaline-2,3-dione

¹H-NMR (400 MHz, DMSO-D₆) δ_{H}: 1.30 (3H, broad), 2.21 (3H, s), 4.30 (2H, broad), 4.48 (2H, broad), 5.98 (1H, broad), 6.10 (1H, broad), 7.00 (1H, d, J=8.5 Hz), 7.36 (1H, broad), 7.48-7.58 (1H, broad, Z/E forms), 7.58-7.70 (1H, broad, Z/E forms), 9.22-9.43 (1H, broad, Z/E forms), 11.50 (2H, broad). LCMS t_{R} (min): 1.54. MS (APCI), m/z 410.04 [M+H]⁺. HPLC t_{R} (min): 9.20. Mₚ 207-209° C.

### 97. 6-Ethoxy-N-furan-2-ylmethyl-N′-[3-(2-methyl-imidazol-1-yl)-phenyl]-[1,3,5]triazine-2,4-diamine

¹H-NMR (400 MHz, DMSO-D₆) δ_{H}: 1.24 (3H, t, J=7.5 Hz), 2.21 (3H, s), 4.28 (2H, broad), 4.46 (2H, broad), 6.10-6.25 (1H, broad, Z/E forms), 6.28-6.40 (1H, broad, Z/E forms), 6.86 (1H, s), 6.98 (1H, broad), 7.19 (1H, d, J=8.5 Hz), 7.38 (1H, t, J=8.5 Hz), 7.50 (1H, d, J=8.5 Hz), 7.76 (1H, broad), 7.87 (2H, broad), 9.50-9.70 (1H, broad, Z/E forms). LCMS t_{R} (min): 1.46. MS (APCI), m/z 392.18 [M+H]⁺. HPLC t_{R} (min): 9.32.

### 98. 2-(3-{4-Ethoxy-6-[(furan-2-ylmethyl)-amino]-[1,3,5]triazin-2-ylamino}-phenyl)-5-methyl-2,4-dihydro-pyrazol-3-one hydrochloride

¹H-NMR (400 MHz, DMSO-D₆) δ_{H}: 1.27 (3H, broad), 2.22 (3H, broad), 3.60 (1H, broad), 4.30 (4H, m), 4.49 (2H, broad), 6.11 (1H, broad), 6.13-6.28 (1H, broad), 6.38 (1H, broad), 6.97 (1H, broad), 7.15 (1H, broad), 7.27 (1H, broad), 7.33 (1H, broad), 7.53 (1H, s), 8.61 (1H, broad). LCMS t_{R} (min): 1.66. MS (APCI), m/z 408.01 [M+H]⁺. HPLC t_{R} (min): 10.90. Mₚ 141-143° C.

### 99. N-[3-(1H-Benzoimidazol-2-yl)-phenyl]-6-ethoxy-N′-furan-2-ylmethyl-[1,3,5]triazine-2,4-diamine hydrochloride

¹H-NMR (400 MHz, DMSO-D₆) δ_{H}: 1.30 (3H, broad), 3.40 (2H, broad), 4.36 (2H, broad), 4.52 (2H, broad), 6.18-6.34 (1H, broad, Z/E forms), 6.25-6.42 (1H, broad, Z/E forms), 7.52 (3H, broad), 7.60 (1H, t, J=8.5 Hz), 7.73-7.90 (4H, broad, Z/E forms), 7.90-8.29 (1H, broad, Z/E forms), 8.54-8.82 (1H, broad, Z/E forms), 9.70-9.87 (1H, broad, Z/E forms). LCMS t_{R} (min): 1.56. MS (APCI), m/z 428.18 [M+H]⁺. HPLC t_{R} (min): 10.42. Mₚ 127-129° C.

### 100. 6-Ethoxy-N-furan-2-ylmethyl-N′-quinoxalin-6-yl-[1,3,5]triazine-2,4-diamine

¹H-NMR (400 MHz, DMSO-D₆) δ_{H}: 1.30 (3H, t, J=7.5 Hz), 4.38 (2H, broad), 4.48-4.63 (2H, broad, Z/E forms), 6.22-6.45 (1H, broad, Z/E forms), 6.40 (1H, broad), 7.57 (1H, s), 7.98 (1H, d, J=8.5 Hz), 8.04 (1H, broad), 8.12 (1H, broad), 8.70 (1H, broad), 8.75 (1H, s), 8.84 (1H, s), 9.89-10.05 (1H, broad, Z/E forms). LCMS t_{R} (min): 1.68. MS (APCI), m/z 364.10 [M+H]⁺. HPLC t_{R} (min): 11.30. M_{P} 165-168° C. (decomp.).

### 101. N-(2-Bromo-phenyl)-6-ethoxy-N′-furan-2-ylmethyl-[1,3,5]triazine-2,4-diamine

¹H-NMR (400 MHz, DMSO-D₆) δ_{H}: 1.28 (3H, t, J=7.5 Hz), 4.24 (2H, broad), 4.41 (2H, broad), 6.10-6.31 (1H, broad, Z/E forms), 6.37 (1H, broad), 7.10 (1H, t, J=8.5 Hz), 7.37 (1H, t, J=8.5 Hz), 7.52 (1H, s), 7.63 (1H, d, J=8.5 Hz), 7.76 (2H, broad), 8.40-8.58 (1H, broad, Z/E forms). LCMS t_{R} (min): 1.94. MS (APCI), m/z 389.93, 391.90 [M+H]⁺. HPLC t_{R} (min): 14.33. Mₚ 110-112° C.

### 102. 6-Ethoxy-N-furan-2-ylmethyl-N′-(2-pyridin-3-yl-phenyl)-[1,3,5]triazine-2,4-diamine

A mixture of compound 101 (390 mg, 1.0 mmol), 3-pyridyl boronic acid (123 mg, 1.0 mmol), Pd(PPh₃)₄ (116 mg, 0.1 mmol), Na₂CO₃ (440 mg, 4.0 mmol), dimethoxy ethane (2 mL) and water (2 mL) was stirred at refluxing for 4 hours under argon atmosphere, cooled to room temperature, diluted with water (20 mL) and extracted with dichloromethane (2×20 mL). The combined organic phases were combined, dried over sodium sulfate and concentrated. Purification by column chromatography (silica gel, acetone/dichloromethane) and preparative TLC (actone/dichloromethane) gave a final compound. ¹H-NMR (400 MHz, DMSO-D₆) δ_{H}: 1.20 (3H, broad), 4.16 (2H, broad), 4.25-4.40 (2H, broad, Z/E forms), 6.00-6.20 (1H, broad, Z/E forms), 6.33 (1H, broad), 7.35 (4H, broad), 7.49 (2H, m), 7.56 (1H, broad), 7.75 (1H, broad), 8.48 (1H, broad), 8.57 (1H, broad), 8.60-8.80 (1H, broad, Z/E forms). LCMS t_{R} (min): 1.47. MS (APCI), m/z 388.92 [M+H]⁺. HPLC t_{R} (min): 9.23. Mₚ 90-92° C.

### 103. 6-{4-Ethoxy-6-[(5-methyl-furan-2-ylmethyl)-amino]-[1,3,5]triazin-2-ylamino}-3H-quinazolin-4-one

¹H-NMR (400 MHz, DMSO-D₆) δ_{H}: 1.30 (3H, broad), 2.21 (3H, s), 4.32 (2H, broad), 4.40-4.55 (2H, broad, Z/E forms), 5.94 (1H, broad), 6.05-6.25 (1H, broad, Z/E forms), 7.57 (1H, d, J=8.5 Hz), 7.70-7.90 (1H, broad, Z/E forms), 7.94 (1H, s), 8.05-8.30 (1H, broad, Z/E forms), 8.40-8.75 (1H, broad, Z/E forms), 9.65-9.80 (1H, broad, Z/E forms), 11.98 (1H, broad). LCMS t_{R} (min): 1.60. MS (APCI), m/z 394.01 [M+H]⁺. HPLC t_{R} (min): 10.07. M_{P} 256-258° C.

### 105. 5-{4-Ethoxy-6-[(furan-2-ylmethyl)-amino]-[1,3,5]triazin-2-ylamino}-1,3-dimethyl-1,3-dihydro-benzoimidazol-2-one

¹H-NMR (400 MHz, DMSO-D₆) δ_{H}: 1.29 (3H, t, J=7.5 Hz), 3.30 (6H, s), 4.30 (2H, broad), 4.50 (2H, broad), 6.22 (1H, broad), 6.35 (1H, broad), 6.99 (1H, d, J=8.5 Hz), 7.22 (1H, d, J=8.5 Hz), 7.52 (1H, s), 7.73 (2H, broad), 9.15-9.40 (1H, broad, Z/E forms). LCMS t_{R} (min): 1.60. MS (APCI), m/z 396.09 [M+H]⁺. HPLC t_{R} (min): 9.97. Mₚ 290-293° C.

### 106. 6-Ethoxy-N-(5-methyl-furan-2-ylmethyl)-N′-[3-(4-methyl-piperazin-1-yl)-phenyl]-[1,3,5]triazine-2,4-diamine

¹H-NMR (400 MHz, DMSO-D₆) δ_{H}: 1.30 (3H, broad triplet, J=7.5 Hz), 2.20 (6H, m), 2.41 (4H, broad), 3.08 (4H, broad), 4.30 (2H, broad), 4.38-4.52 (2H, broad, Z/E forms), 5.95 (1H, broad), 6.10 (1H, broad), 6.55 (1H, broad doublet, J=8.5 Hz), 7.05-7.27 (1H, broad, Z/E forms), 7.08 (1H, broad), 7.30-7.55 (1H, broad, Z/E forms), 7.70 (1H, broad), 9.03-9.28 (1H, broad, Z/E forms). LCMS t_{R} (min): 1.51. MS (APCI), m/z 424.16 [M+H]⁺. HPLC t_{R} (min): 9.40. M_{P} 127-129° C.

### 107. 6-Ethoxy-N-(2-ethoxy-3H-benzoimidazol-5-O—N′-furan-2-ylmethyl-[1,3,5]triazine-2,4-diamine

¹H-NMR (400 MHz, DMSO-D₆) δ_{H}: 1.28 (3H, t, J=7.5 Hz), 1.38 (3H, t, J=7.5 Hz), 4.30 (2H, broad), 4.40-4.54 (4H, broad), 6.20-6.34 (1H, broad, Z/E forms), 6.38 (1H, broad), 7.08-7.22 (1H, two d, J=8.5 Hz, Z/E forms), 7.29 (1H, broad), 7.52 (1H, s), 7.65 (1H, broad), 7.78 (1H, broad), 9.05-9.30 (1H, broad, Z/E forms), 11.50-11.62 (1H, broad, Z/E forms). LCMS t_{R} (min): 1.57. MS (APCI), m/z 396.11 [M+H]⁺. HPLC t_{R} (min): 9.03. M_{P} 169-171° C.

### 108. 6-Ethoxy-N-furan-2-ylmethyl-N′-m-tolyl-[1,3,5]triazine-2,4-diamine

¹H-NMR (400 MHz, DMSO-D₆) δ_{H}: 1.28 (3H, t, J=7.5 Hz), 2.26 (3H, s), 4.30 (2H, d, J=7.5 Hz), 4.49 (2H, broad), 6.22 (1H, broad), 6.37 (1H, broad), 6.78 (1H, d, J=8.5 Hz), 7.13 (1H, t, J=8.5 Hz), 7.48 (1H, broad), 7.54 (1H s), 7.58 (1H, broad), 7.75 (1H, broad), 9.10-9.40 (1H, broad, Z/E forms). LCMS t_{R} 1.87 (min). MS (APCI), m/z 326.01 [M+H]⁺. Mₚ 147-148° C.

### 109. 2,4-Dichloro-N-{4-ethoxy-6-[(furan-2-ylmethyl)-amino]-[1,3,5]triazin-2-ylmethyl}-benzamide

Yield 43 mg, 8%. ¹H-NMR (400 MHz, DMSO-D₆) δ_{H}: 1.30 (3H, t, J=7.5 Hz), 4.20-4.35 (2H, two doublets, J=7.5 Hz, Z/E forms), 4.35 (2H, q, J=7.5 Hz), 4.45-4.60 (2H, two doublets, J=7.5 Hz, Z/E forms), 6.26 (1H, broad), 6.38 (1H, broad), 7.45-7.55 (1H, d=8.5 Hz), 7.45-7.6.3 (1H, two doublets, J=8.5 Hz, Z/E forms), 7.56 (1H, s), 7.68 (1H, s), 8.22-8.35 (1H, broad, Z/E forms), 8.68-8.80 (1H, broad, Z/E forms). LCMS t_{R} (min): 1.78. MS (APCI), m/z 421.98, 423.89 [M+H]⁺. HPLC t_{R} (min): 13.46. M_{P} 156-158° C.

TABLE 10

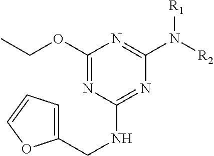

| En | Structure | IUPAC Name | MW | Formula |
|---|---|---|---|---|
| 1 | 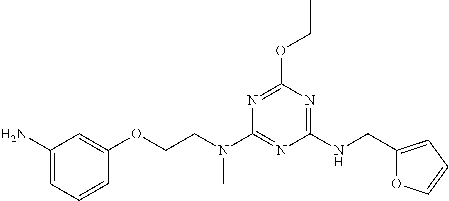 | N-[2-(3-aminophenoxy)ethyl]-6- ethoxy-N′-(furan-2-ylmethyl)-N- methyl-1,3,5-triazine-2,4-diamine | 384.4 | C22H21N7O3 |
| 2 | 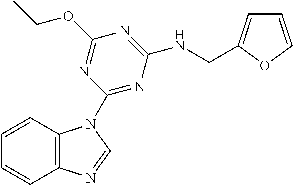 | 4-(1H-benzimidazol-1-yl)-6- ethoxy-N-(furan-2-ylmethyl)-1,3,5- triazin-2-amine | 336.3 | C16H15ClN4O2 |
| 3 | 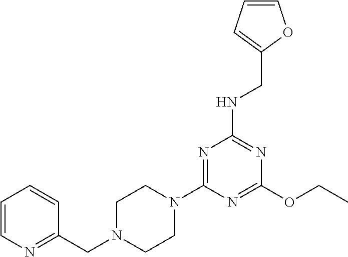 | 4-ethoxy-N-(furan-2-ylmethyl)-6- [4-(pyridin-2-ylmethyl)piperazin-1- yl]-1,3,5-triazin-2-amine | 395.5 | C21H20N6O2 |
| 4 | 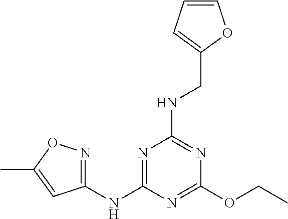 | 6-ethoxy-N-(furan-2-ylmethyl)-N′- (5-methylisoxazol-3-yl)-1,3,5- triazine-2,4-diamine | 316.3 | C18H17Cl2N5O3 |
| 5 | 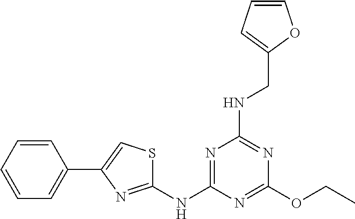 | 6-ethoxy-N-(furan-2-ylmethyl)-N′- (4-phenyl-1,3-thiazol-2-yl)-1,3,5- triazine-2,4-diamine | 394.5 | C17H26N6O2 |
| 6 | 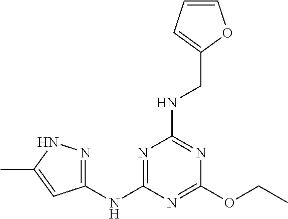 | 6-ethoxy-N-(furan-2-ylmethyl)-N′- (3-methyl-1H-pyrazol-5-yl)-1,3,5- triazine-2,4-diamine | 315.3 | C22H29N7O2 |
| 7 | 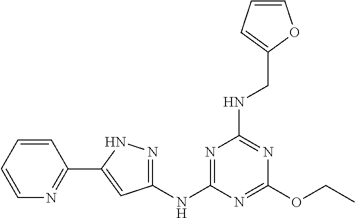 | 6-ethoxy-N-(furan-2-ylmethyl)-N′- [3-(pyridin-2-yl)-1H-pyrazol-5-yl]- 1,3,5-triazine-2,4-diamine | 378.4 | C17H16N6O2 |
| 8 | 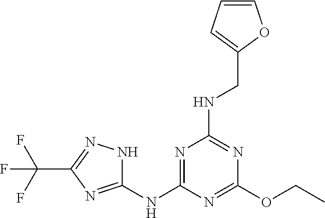 | 6-ethoxy-N-(furan-2-ylmethyl)-N′- [5-(trifluoromethyl)-1H-1,2,4- triazol-3-yl]-1,3,5-triazine-2,4- diamine | 370.3 | C16H23N5O3 |
| 9 | 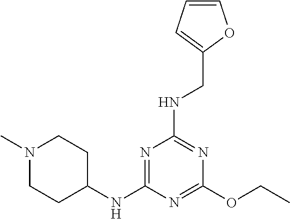 | 6-ethoxy-N-(furan-2-ylmethyl)-N′- (1-methylpiperidin-4-yl)-1,3,5- triazine-2,4-diamine | 332.4 | C14H16N6O3 |
| 10 | 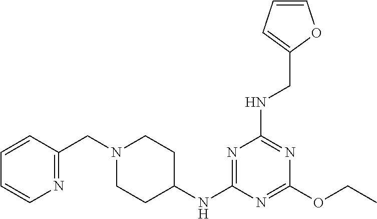 | 6-ethoxy-N-(furan-2-ylmethyl)-N′- [1-(pyridin-2-ylmethyl)piperidin-4- yl]-1,3,5-triazine-2,4-diamine | 409.5 | C20H25N7O2 |
| 11 | 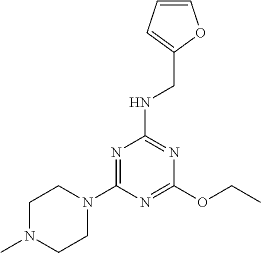 | 4-ethoxy-N-(furan-2-ylmethyl)-6- (4-methylpiperazin-1-yl)-1,3,5- triazin-2-amine | 318.4 | C20H21N7O2 |
| 12 | 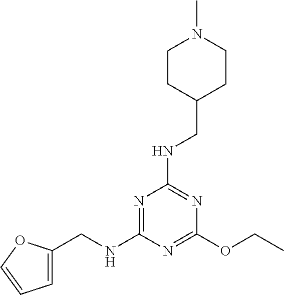 | 6-ethoxy-N-(furan-2-ylmethyl)-N′- [(1-methylpiperidin-4-yl)methyl]- 1,3,5-triazine-2,4-diamine | 346.4 | C13H14N6O2S |
| 13 | 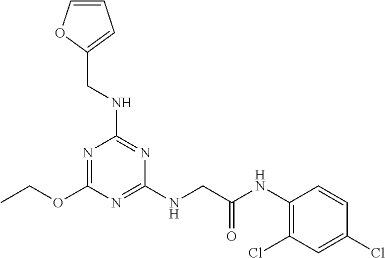 | N-(2,4-dichlorophenyl)-N~2~-{4- ethoxy-6-[(furan-2- ylmethyl)amino]-1,3,5-triazin-2- yl}glycinamide | 437.3 | C18H18N8O2 |
| 14 | 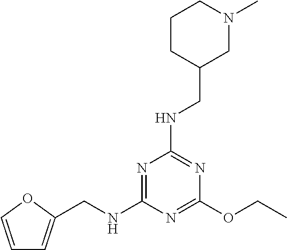 | 6-ethoxy-N-(furan-2-ylmethyl)-N′- [(1-methylpiperidin-3-yl)methyl]- 1,3,5-triazine-2,4-diamine | 346.4 | C16H24N6O2 |
| 15 | 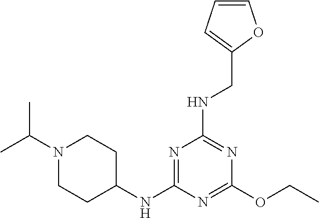 | 6-ethoxy-N-(furan-2-ylmethyl)-N′- [1-(propan-2-yl)piperidin-4-yl]- 1,3,5-triazine-2,4-diamine | 360.5 | C13H13F3N8O2 |
| 16 | 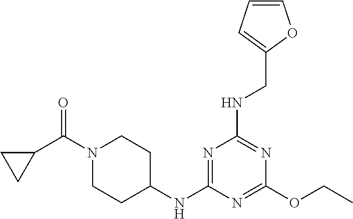 | cyclopropyl[4-({4-ethoxy-6-[(furan- 2-ylmethyl)amino]-1,3,5-triazin-2- yl}amino)piperidin-1-yl]methanone | 386.4 | C16H21N7O2 |
| 17 | 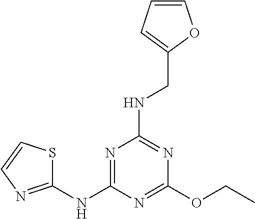 | 6-ethoxy-N-(furan-2-ylmethyl)-N′- (1,3-thiazol-2-yl)-1,3,5-triazine- 2,4-diamine | 318.4 | C17H20N6O2 |
| 18 | 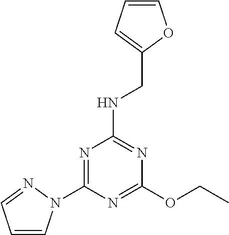 | 4-ethoxy-N-(furan-2-ylmethyl)-6- (1H-pyrazol-1-yl)-1,3,5-triazin-2- amine | 286.3 | C14H17N7O2 |
| 19 | 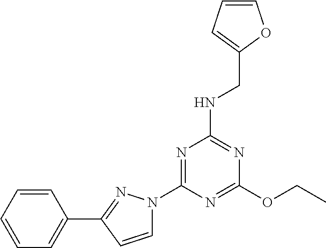 | 4-ethoxy-N-(furan-2-ylmethyl)-6- (3-phenyl-1H-pyrazol-1-yl)-1,3,5- triazin-2-amine | 362.4 | C10H11ClN4O2 |
| 20 | 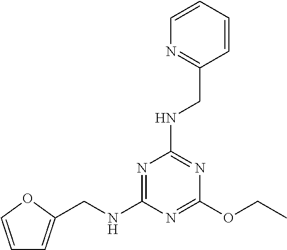 | 6-ethoxy-N-(furan-2-ylmethyl)-N′- (pyridin-2-ylmethyl)-1,3,5-triazine- 2,4-diamine | 326.4 | C13H14N6O2 |
| 21 | 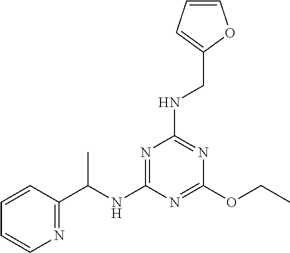 | 6-ethoxy-N-(furan-2-ylmethyl)-N′- [1-(pyridin-2-yl)ethyl]-1,3,5- triazine-2,4-diamine | 340.4 | C18H28N6O2 |
| 22 | 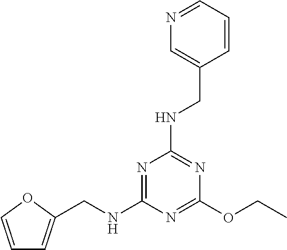 | 6-ethoxy-N-(furan-2-ylmethyl)-N′- (pyridin-3-ylmethyl)-1,3,5-triazine- 2,4-diamine | 326.4 | C19H26N6O3 |
| 23 | 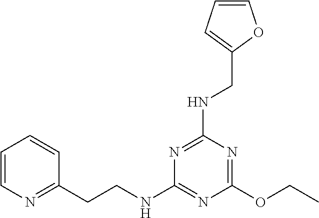 | 6-ethoxy-N-(furan-2-ylmethyl)-N′- [2-(pyridin-2-yl)ethyl]-1,3,5- triazine-2,4-diamine | 340.4 | C16H18N6O2 |
| 24 | 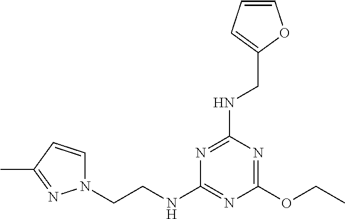 | 6-ethoxy-N-(furan-2-ylmethyl)-N′- [2-(3-methyl-1H-pyrazol-1- yl)ethyl]-1,3,5-triazine-2,4-diamine | 343.4 | C19H18N6O2S |
| 25 | 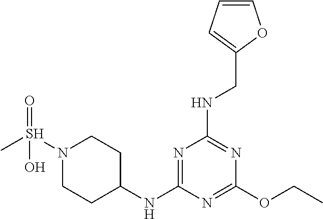 | 6-ethoxy-N-(furan-2-ylmethyl)-N′- [1-(methylsulfonyl)piperidin-4-yl]- 1,3,5-triazine-2,4-diamine | 397.5 | C19H21N7O3 |

### Procedures and Analytical Data for Compounds in Table 10.

### 1. N-[2-(3-Amino-phenoxy)-ethyl]-6-ethoxy-N′-furan-2-ylmethyl-N-methyl-[1,3,5]triazine-2,4-diamine

Yield 176 mg, 39%. ¹H-NMR (400 MHz, DMSO-D₆) δ_{H}: 1.27 (3H, t, J=7.5 Hz), 3.12 (3H, broad), 3.84 (2H, broad), 4.03 (2H, broad), 4.25 (2H, broad), 4.43 (2H, broad), 4.90 (2H, broad), 6.08 (1H, d, J=8.5 Hz), 6.17 (2H, m), 6.20 (1H, broad), 6.34 (1H, broad), 6.88 (1H, t, J=8.5 Hz), 7.50 (2H, broad). LCMS t_{R} (min): 1.53. MS (APCI), m/z 385.09 [M+H]⁺. HPLC t_{R} (min): 8.72. Mₚ 34-36° C.

### 3. [4-Ethoxy-6-(4-pyridin-2-ylmethyl-piperazin-1-yl)-[1,3,5]triazin-2-yl]-furan-2-ylmethyl-amine

A mixture of 4-Ethoxy-6-chloro-[1,3,5]triazin-2-yl]-furan-2-ylmethyl-amine (300 mg, 1.18 mmol), 1-Pyridin-2-ylmethyl-piperazine (230 mg, 1.30 mmol), NEt₃ (360 mg, 3.54 mmol) and acetonitrile (6 mL) was stirred at refluxing for 3 hours, cooled to room temperature, diluted with water, extracted with chloroform. The combined organic phases were concentrated. Purification by column chromatography (silica gel, ethyl acetate/hexane) gave the compound. Yield 270 mg, 58%. ¹H-NMR (400 MHz, DMSO-D₆) δ_{H}: 1.22 (3H, t, J=7.5 Hz), 2.42 (4H, m), 3.61 (2H s), 3.70 (4H, broad), 4.22 (2H, broad), 4.40 (2H, broad doublet, J=7.5 Hz), 6.19 (1H, broad), 6.33 (1H, broad), 7.24 (1H, broad), 7.44 (1H, d, J=8.0 Hz), 7.49 (1H, s), 7.53 (1H, broad), 7.74 (1H, t, J=8.5 Hz), 8.48 (1H, broad). LCMS t_{R} (min): 1.37. MS (APCI), m/z 396.14 [M+H]⁺. HPLC t_{R} (min): 8.23. Mₚ 115-117° C.

### 4. 6-Ethoxy-N-furan-2-ylmethyl-N′-(5-methyl-isoxazol-3-yl)-[1,3,5]triazine-2,4-diamine

Yield 110 mg, 35%. ¹H-NMR (400 MHz, DMSO-D₆) δ_{H}: 1.29 (3H, broad triplet, J=7.5 Hz), 2.35 (3H, s), 4.30 (2H, broad), 4.50 (2H, broad doublet, J=7.5 Hz), 6.28 (1H, broad), 6.40 (1H, broad), 6.60-6.90 (1H, broad, Z/E forms), 7.55 (1H, broad), 7.96 (1H, broad triplet, J=7.5 Hz), 10.00-10.16 (1H, broad, Z/E forms). LCMS t_{R} (min): 1.74. MS (APCI), m/z 317.06 [M+H]⁺. HPLC t_{R} (min): 11.98. M_{P} 194-196° C.

### 5. 6-Ethoxy-N-furan-2-ylmethyl-N′-(4-phenyl-thiazol-2-yl)-[1,3,5]triazine-2,4-diamine

Yield 63 mg, 11%. ¹H-NMR (400 MHz, DMSO-D₆) δ_{H}: 1.32 (3H, broad triplet, J=7.5 Hz, Z/E forms), 4.39 (2H, broad), 4.48-4.70 (2H, broad, Z/E forms), 6.22-6.35 (1H, broad, Z/E forms), 6.40 (1H, broad), 7.30 (1H, t, J=8.5 Hz), 7.41 (2H, t, J=8.5 Hz), 7.54 (1H, s), 7.90 (2H, d, J=8.5 Hz), 8.03-8.22 (1H, broad, Z/E forms), 11.38-11.57 (1H, broad, Z/E forms). LCMS t_{R} (min): 2.00. MS (APCI), m/z 395.08 [M+H]⁺. HPLC t_{R} (min): 14.61. M_{P} 204-206° C.

### 6. 6-Ethoxy-N-furan-2-ylmethyl-N′-(5-methyl-2H-pyrazol-3-yl)-[1,3,5]triazine-2,4-diamine

Yield 80 mg, 26%. ¹H-NMR (400 MHz, DMSO-D₆) δ_{H}: 1.29 (3H, broad triplet, J=7.5 Hz), 2.18 (3H, s), 4.29 (2H, broad q, J=7.5 Hz), 4.48 (2H, broad), 6.22 (2H, broad), 6.39 (1H, broad), 7.52 (1H, s), 7.70 (1H, broad), 9.27 (1H, broad), 11.80 (1H, broad). LCMS t_{R} (min): 1.55. MS (APCI), m/z 316.11 [M+H]⁺. HPLC t_{R} (min): 9.47. M_{P} 200-202° C.

### 7. 6-Ethoxy-N-furan-2-ylmethyl-N′-(5-pyridin-2-O-2H-pyrazol-3-yl)-[1,3,5]triazine-2,4-diamine

Yield 137 mg, 31%. ¹H-NMR (400 MHz, DMSO-D₆) δ_{H}: 1.30 (3H, broad triplet, J=7.5 Hz), 4.33 (2H, broad), 4.53 (2H, broad), 6.30 (1H, broad), 6.35-6.48 (1H, broad, Z/E forms), 7.10-7.40 (1H, broad, Z/E forms), 7.32 (1H, broad), 7.51-7.63 (1H, broad, Z/E forms), 7.70-7.85 (1H, broad, Z/E forms), 7.90 (1H, broad), 7.95-8.20 (1H, broad, Z/E forms), 8.52-8.68 (1H, broad, Z/E forms), 9.43-10.38 (1H, broad, Z/E forms), 12.87 (1H, broad). LCMS t_{R} (min): 1.55. MS (APCI), m/z 379.14 [M+H]⁺. HPLC t_{R} (min): 9.50. M_{P} 198-200° C.

### 8. 6-Ethoxy-N-furan-2-ylmethyl-N′-(5-trifluoromethyl-1H-[1,2,4]triazol-3-yl)-[1,3,5]triazine-2,4-diamine

Yield 200 mg, 46%. ¹H-NMR (400 MHz, DMSO-D₆) δ_{H}: 1.32 (3H, broad triplet, J=7.5 Hz), 4.42 (2H, superposition of two quartets, J=7.5 Hz), 4.59 (2H, superposition of two doublets, J=7.5 Hz), 6.35 (1H, broad), 6.41 (1H, broad), 7.53-7.62 (1H, two s, Z/E forms), 7.92-8.20 (1H, broad Z/E forms), 8.15 (1H, broad), 8.80-8.95 (1H, broad Z/E forms). LCMS t_{R} (min): 1.73. MS (APCI), m/z 371.04 [M+H]⁺. HPLC t_{R} (min): 12.73. M_{P} 289-291° C.

### 9. 6-Ethoxy-N-furan-2-ylmethyl-N′-(1-methyl-piperidin-4-yl)-[1,3,5]triazine-2,4-diamine

Yield 87 mg, 28%. ¹H-NMR (400 MHz, DMSO-D₆) δ_{H}: 1.23 (3H, broad), 1.50 (2H, broad), 1.75 (2H, m), 1.96 (2H, m), 2.17 (3H, s), 2.74 (2H, m), 3.68 (1H, broad), 4.22 (2H, broad), 4.42 (2H, broad), 6.20 (1H, broad), 6.36 (1H, broad), 6.85-7.08 (1H, broad, Z/E forms), 7.22-7.55 (1H, broad, Z/E forms), 7.50 (1H, s). LCMS t_{R} (min): 1.30. MS (APCI), m/z 333.16 [M+H]⁺. HPLC t_{R} (min): 7.16. M_{P} 125-127° C.

### 10. 6-Ethoxy-N-furan-2-ylmethyl-N′-(1-pyridin-2-ylmethyl-piperidin-4-yl)-[1,3,5]triazine-2,4-diamine

A mixture of compound 24 (300 mg, 1.18 mmol), 1-pyridin-2-ylmethyl-piperidin-4-ylamine dihydrochloride (311 mg, 1.18 mmol), DIPEA (460 mg, 3.54 mmol) and acetonitrile (6 mL) was stirred at room temperature for 4 hours, diluted with water. The formed solid was collected by filtration. Purification by prepTLC (10% ethanol/ethyl acetate) gave compound 26ae.

Yield 104 mg, 34%. ¹H-NMR (400 MHz, DMSO-D₆) δ_{H}: 1.21 (3H, broad), 1.50 (2H, m), 1.76 (2H, m), 2.10 (2H, broad), 2.81 (2H, m), 3.60 (2H, s), 3.72 (1H, broad), 4.21 (2H, broad), 4.40 (2H, broad), 6.18 (1H, broad), 6.33 (1H, broad), 6.90-7.08 (1H, broad, Z/E forms), 7.24 (1H, dd, J=8.0, 5.0 Hz), 7.25-7.50 (1H, broad, Z/E forms), 7.41 (1H, d, J=8.0 Hz), 7.50 (1H, s), 7.76 (1H, d, J=8.0 Hz), 8.48 (1H, broad). LCMS t_{R} (min): 1.34. MS (APCI), m/z 410.17 [M+H]⁺. HPLC t_{R} (min): 7.88.

### 11. [4-Ethoxy-6-(4-methyl-piperazin-1-yl)-[1,3,5]triazin-2-yl]-furan-2-ylmethyl-amine CF₃COOH

Yield 86 mg, 10%. ¹H-NMR (400 MHz, DMSO-D₆) δ_{H}: 1.25 (3H, t, J=7.5 Hz), 2.81 (3H, s), 3.01 (2H, broad), 3.21 (2H, broad), 3.45 (2H, broad), 4.27 (2H, broad), 4.44 (2H, broad), 4.61 (2H, broad), 6.22 (1H, broad), 6.38 (1H, broad), 7.52 (1H, s), 7.38-7.85 (1H, broad, Z/E forms), 9.85 (1H, broad). LCMS t_{R} (min): 1.28. MS (APCI), m/z 319.10 [M+H]⁺. HPLC t_{R} (min): 7.59.

### 12. 6-Ethoxy-N-furan-2-ylmethyl-N′-(1-methyl-piperidin-4-ylmethyl)-[1,3,5]triazine-2,4-diamine (9b)

Yield 100 mg, 25%. ¹H-NMR (400 MHz, DMSO-D₆) δ_{H}: 1.13 (2H, m), 1.22 (3H, broad), 1.45 (1H, m), 1.60 (2H, m), 1.79 (2H, m), 2.12 (3H, s), 2.71 (2H, m), 3.11 (2H, broad), 4.22 (2H, broad), 4.42 (2H, d, J=7.5 Hz), 6.20 (1H, broad), 6.38 (1H, broad), 7.00-7.20 (1H, broad, Z/E forms), 7.25-7.48 (1H, broad, Z/E forms), 7.52 (1H, s). LCMS t_{R} (min): 1.39. MS (APCI), m/z 347.15 [M+H]⁺. HPLC t_{R} (min): 7.14. M_{P} 154-156° C.

### 13. N-(2,4-Dichloro-phenyl)-2-{4-ethoxy-6-[(furan-2-ylmethyl)-amino]-[1,3,5]triazin-2-ylamino}-acetamide

Yield 133 mg, 24%. ¹H-NMR (400 MHz, DMSO-D₆) δ_{H}: 1.22 (3H, broad), 4.07 (2H, broad), 4.23 (2H, broad), 4.42 (2H, broad), 6.20 (1H, broad), 6.20-6.40 (1H, broad, Z/E forms), 7.40 (1H, d, J=8.5 Hz), 7.43 (1H, broad), 7.52 (1H, broad), 7.58 (1H, broad), 7.62 (1H, broad), 7.90 (1H, broad), 9.40 (1H, broad). LCMS t_{R} (min): 1.86. MS (APCI), m/z 437.00, 439.00 [M+H]⁺. HPLC t_{R} (min): 12.38. M_{P} 166-168° C.

### 14. 6-Ethoxy-N-furan-2-ylmethyl-N″-(1-methyl-piperidin-3-ylmethyl)-[1,3,5]triazine-2,4-diamine

Yield 150 mg, 22%. ¹H-NMR (400 MHz, DMSO-D₆) δ_{H}: 0.91 (1H, broad), 1.23 (3H, broad), 1.47 (1H, broad), 1.62 (2H, broad), 1.80 (2H, broad), 2.00 (1H, broad), 2.21 (3H, s), 2.63-2.80 (2H, broad, Z/E forms), 3.11 (2H, broad), 4.22 (2H, broad), 4.42 (2H, broad), 6.21 (1H, broad), 6.37 (1H, broad), 7.00-7.22 (1H, broad, Z/E forms), 7.28-7.50 (1H, broad, Z/E forms), 7.52 (1H, s). LCMS t_{R} (min): 1.32. MS (APCI), m/z 347.20 [M+H]⁺. HPLC t_{R} (min): 7.31.

### 15. 6-Ethoxy-N-furan-2-ylmethyl-N′-(1-isopropyl-piperidin-4-yl)-[1,3,5]triazine-2,4-diamine

Yield 150 mg, 35%. ¹H-NMR (400 MHz, DMSO-D₆) δ_{H}: 0.92 (6H, d, J=7.5 Hz), 1.20 (3H, broad), 1.40 (2H, m), 1.76 (2H, m), 2.11 (2H, m), 2.65 (1H, m), 2.71 (2H, m), 3.63 (1H, broad), 4.20 (2H, broad), 4.40 (2H, broad), 6.18 (1H, broad), 6.34 (1H, broad), 6.93 (1H, broad), 7.25-7.43 (1H, broad, Z/E forms), 7.50 (1H, s). LCMS t_{R} (min): 1.34. MS (APCI), m/z 361.16 [M+H]⁺. HPLC t_{R} (min): 7.52. M_{P} 136-138° C.

### 16. Cyclopropyl-(4-{4-ethoxy-6-[(furan-2-ylmethyl)-amino]-[1,3,5]triazin-2-ylamino}-piperidin-1-yl)-methanone

Yield 215 mg, 47%. ¹H-NMR (400 MHz, DMSO-D₆) δ_{H}: 0.70 (4H, broad m), 1.23 (3H, broad), 1.36 (2H, broad), 1.80 (2H, broad), 1.95 (1H, broad), 2.73 (1H, broad), 3.15 (1H, broad), 3.98 (1H, broad), 4.24 (4H, broad), 4.42 (2H, broad), 6.21 (1H, broad), 6.37 (1H, broad), 6.96-7.20 (1H, broad, Z/E forms), 7.20-7.60 (1H, broad Z/E forms), 7.53 (1H, s). LCMS t_{R} (min): 1.47. MS (APCI), m/z 387.06 [M+H]⁺. HPLC t_{R} (min): 9.35. M_{P} 153-154° C.

### 17. 6-Ethoxy-N-furan-2-ylmethyl-N′-thiazol-2-yl-[1,3,5]triazine-2,4-diamine

Yield 77 mg, 21%. ¹H-NMR (400 MHz, DMSO-D₆) δ_{H}: 1.30 (3H, broad triplet, J=7.5 Hz), 4.38 (2H, broad), 4.60 (2H, broad), 6.29 (1H, broad), 6.39 (1H, broad), 7.05 (1H, broad), 7.35 (1H, broad), 7.52 (1H, s), 7.82 (1H, broad), 10.50 (1H, broad). LCMS t_{R} (min): 1.65. MS (APCI), m/z 319.04 [M+H]⁺. HPLC t_{R} (min): 10.54. M_{P} 252-254° C.

### 18. (4-Ethoxy-6-pyrazol-1-yl)-[1,3,5]triazin-2-yl)-furan-2-ylmethyl-amine

Yield 158 mg, 47%. ¹H-NMR (400 MHz, DMSO-D₆) δ_{H}: 1.34 (3H, broad), 4.40 (2H, superposition of two q, J=7.5 Hz), 4.50-4.65 (2H, superposition of two doublets, J=7.5 Hz, Z/E forms), 6.26-6.40 (1H, broad, Z/E forms), 6.39 (1H, broad), 6.58 (1H, broad), 7.55 (1H, s), 7.78-7.90 (1H, broad Z/E forms), 8.48-8.58 (1H, broad Z/E forms), 8.58-8.80 (1H, broad Z/E forms). LCMS t_{R} (min): 1.64. MS (APCI), m/z 287.03 [M+H]⁺. HPLC t_{R} (min): 11.64.

### 19. [4-Ethoxy-6-(3-phenyl-pyrazol-1-yl)-[1,3,5]-triazin-2-yl]-furan-2-ylmethyl-amine

Yield 265 mg, 62%. ¹H-NMR (400 MHz, DMSO-D₆) δ_{H}: 1.33 (3H, broad), 4.35-4.50 (2H, two q, J=7.5 Hz, Z/E forms), 4.50-4.69 (2H, two d, J=7.5 Hz, Z/E forms), 6.28-6.40 (1H, broad, Z/E forms), 6.35-6.44 (1H, broad, Z/E forms), 7.07 (1H, broad), 7.40 (1H, broad), 7.48 (2H, m), 7.56 (1H, s), 7.92 (2H, broad), 8.59 (1H, broad), 8.67-8.87 (1H, broad, Z/E forms). LCMS t_{R} (min): 2.54. MS (APCI), m/z 363.09 [M+H]⁺. HPLC t_{R} (min): 15.30. M_{P} 172-174° C.

### 20. 6-Ethoxy-N-furan-2-ylmethyl-N′-pyridin-2-ylmethyl-[1,3,5]triazine-2,4-diamine

Yield 110 mg, 27%. ¹H-NMR (400 MHz, DMSO-D₆) δ_{H}: 1.12-1.30 (3H, broad, Z/E forms), 4.10-4.27 (2H, broad, Z/E forms), 4.27-4.49 (2H, broad, Z/E forms), 4.54 (2H, broad), 5.95-6.21 (1H, broad, Z/E forms), 6.21-6.40 (1H, broad, Z/E forms), 7.22 (1H, dd, J=8.0, 5.0 Hz), 7.27 (1H, d, J=8.0 Hz), 7.42-7.59 (3H, broad, Z/E forms), 7.59-7.77 (1H, broad, Z/E forms), 8.48 (1H, d, J=5.0 Hz). LCMS t_{R} (min): 1.34. MS (APCI), m/z 327.09 [M+H]⁺. HPLC t_{R} (min): 7.42. M_{P} 125-127° C.

### 21. 6-Ethoxy-N-furan-2-ylmethyl-N′-(1-pyridin-2-yl-ethyl)-[1,3,5]triazine-2,4-diamine

Yield 90 mg, 22%. ¹H-NMR (400 MHz, DMSO-D6) OH: 1.10-1.30 (3H, broad Z/E forms), 1.42 (3H, d, J=7.5 Hz), 4.10-4.32 (2H, broad Z/E forms), 4.42 (2H, broad), 5.15 (1H, broad), 6.00-6.20 (1H, broad Z/E forms), 6.27-6.40 (1H, broad Z/E forms), 7.22 (1H, dd, J=8.0, 5.0 Hz), 7.38 (1H, broad), 7.50 (3H, broad), 7.71 (1H, broad), 8.50 (1H, broad). LCMS t_{R} (min): 1.37. MS (APCI), m/z 341.09 [M+H]⁺. HPLC t_{R} (min): 7.79.

### 22. 6-Ethoxy-N-furan-2-ylmethyl-N′-pyridin-3-ylmethyl-[1,3,5]triazine-2,4-diamine

Yield 124 mg, 32%. ¹H-NMR (400 MHz, DMSO-D₆) δ_{H}: 1.20 (3H, broad), 4.20 (2H, broad), 4.41 (4H, broad), 6.00-6.23 (1H, broad, Z/E forms), 6.32 (1H, broad), 7.30 (1H, broad), 7.38-7.55 (1H, broad, Z/E forms), 7.48 (1H, broad), 7.55-7.70 (1H, broad, Z/E forms), 7.66 (1H, broad), 8.41 (1H, broad), 8.50 (1H, broad). LCMS t_{R} (min): 1.31. MS (APCI), m/z 327.09 [M+H]⁺. HPLC t_{R} (min): 7.07. M_{P} 127-129° C.

### 23. 6-Ethoxy-N-furan-2-ylmethyl-N′-(2-pyridin-2-yl-ethyl)-[1,3,5]triazine-2,4-diamine

Yield 250 mg, 62%. ¹H-NMR (400 MHz, DMSO-D₆) δ_{H}: 1.22 (3H, broad), 2.92 (2H, t, J=7.5 Hz), 3.58 (2H, broad q, J=7.5 Hz), 4.20 (2H, broad), 4.40 (2H, broad), 6.20 (1H, broad), 6.32 (1H, broad), 6.95-7.15 (1H, broad Z/E forms), 7.18 (2H, broad), 7.21-7.40 (1H, broad Z/E forms), 7.49 (1H, s), 7.66 (1H, broad triplet, J=8.0 Hz), 8.49 (1H, broad doublet, J=5.0 Hz). LCMS t_{R} (min): 1.31. MS (APCI), m/z 341.08 [M+H]⁺. HPLC t_{R} (min): 7.20. M_{P} 144-146° C.

### 24. 6-Ethoxy-N-furan-2-ylmethyl-N′-[2-(3-methyl-pyrazol-1-yl)-ethyl]-[1,3,5]triazine-2,4-diamine

Yield 140 mg, 41%. ¹H-NMR (400 MHz, DMSO-D₆) δ_{H}: 1.25 (3H, broad), 2.12 (3H, s), 3.59 (2H, broad), 4.12 (2H, t, J=7.5 Hz), 4.23 (2H, broad), 4.42 (2H, broad), 5.96 (1H, s), 6.16-6.30 (1H, broad, Z/E forms), 6.36 (1H, broad), 7.00-7.31 (1H, broad, Z/E forms), 7.40-7.50 (1H, broad, Z/E forms), 7.51 (1H, s), 7.51-7.75 (1H, broad, Z/E forms). LCMS t_{R} (min): 1.52. MS (APCI), m/z 344.08 [M+H]⁺. HPLC t_{R} (min): 9.28. M_{P} 143-145° C.

### 25. 6-Ethoxy-N-furan-2-ylmethyl-N′-(1-methanesulfonyl-piperidin-4-yl)-[1,3,5]triazine-2,4-diamine

Yield 263 mg, 56%. ¹H-NMR (400 MHz, DMSO-D₆) δ_{H}: 1.22 (3H, broad), 1.52 (2H, broad), 1.89 (2H, m), 2.83 (2H, broad), 2.85 (3H, s), 3.52 (2H, m), 3.87 (1H, broad), 4.21 (2H, broad), 4.42 (2H, broad), 6.20 (1H, broad), 6.38 (1H, broad), 7.14 (1H, broad), 7.25-7.48 (1H, broad, Z/E forms), 7.53 (1H, s). MW 396.47. LCMS t_{R} (min): 1.45. MS (APCI), m/z 397.11 [M+H]⁺. HPLC t_{R} (min): 9.44. M_{P} 127-129° C.

TABLE 11

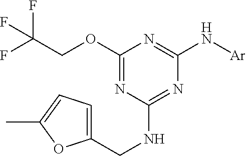

| En | Structure | IUPAC Name | MW | Formula |
|---|---|---|---|---|
| 1 | 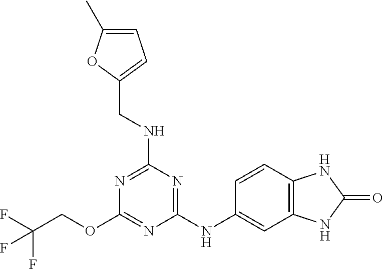 | 5-{[4-{[(5-methylfuran-2- yl)methyl]amino}-6-{2,2,2- trifluoroethoxy)-1,3,5-triazin-2- yl]amino}-1,3-dihydro-2H- benzimidazol-2-one | 435.4 | C18H16F3N7O3 |
| 2 | 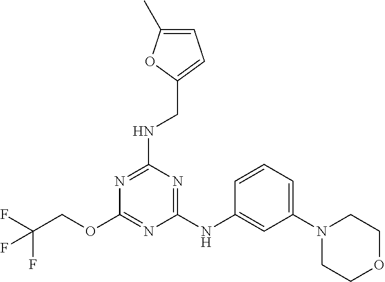 | N-[(5-methylfuran-2-yl)methyl]-N′-[3- (morpholin-4-yl)phenyl]-6-(2,2,2- trifluoroethoxy)-1,3,5-triazine-2,4- diamine | 464.4 | C21H23F3N6O3 |
| 3 | 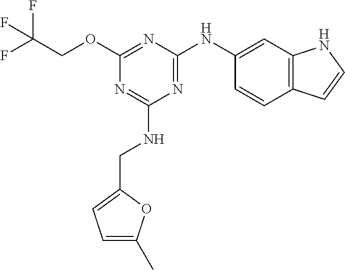 | N-(1H-indol-6-yl)-N′-[(5-methylfuran-2- yl)methyl]-6-(2,2,2-trifluoroethoxy)- 1,3,5-triazine-2,4-diamine | 418.4 | C19H17F3N6O2 |
| 4 | 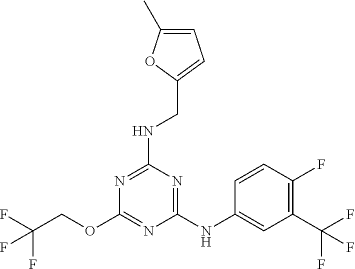 | N-[4-fluoro-3-(trifluoromethyl)phenyl]- N′-[(5-methylfuran-2-yl)methyl]-6- (2,2,2-trifluoroethoxy)-1,3,5-triazine- 2,4-diamine | 465.3 | C18H14F7N5O2 |
| 5 | 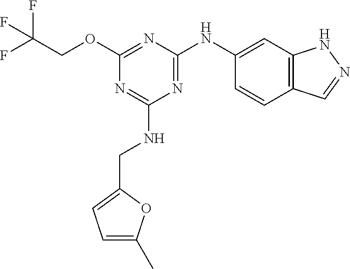 | N-(1H-indazol-6-yl)-N′-[(5- methylfuran-2-yl)methyl]-6-(2,2,2- trifluoroethoxy)-1,3,5-triazine-2,4- diamine | 419.4 | C18H16F3N7O2 |
| 6 | 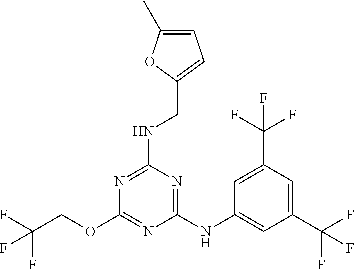 | N-[3,5-bis(trifluoromethyl)phenyl]-N′- [(5-methylfuran-2-yl)methyl]-6-(2,2,2- trifluoroethoxy)-1,3,5-triazine-2,4- diamine | 515.3 | C19H14F9N5O2 |
| 7 | 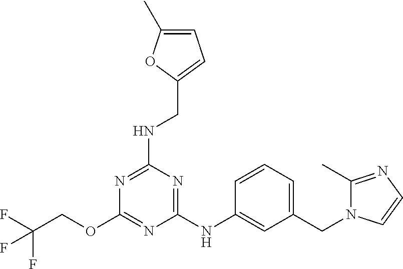 | N-[(5-methylfuran-2-yl)methyl]-N′-{3- [(2-methyl-1H-imidazol-1- yl)methyl]phenyl}-6-(2,2,2- trifluoroethoxy)-1,3,5-triazine-2,4- diamine | 473.5 | C22H22F3N7O2 |
| 8 | 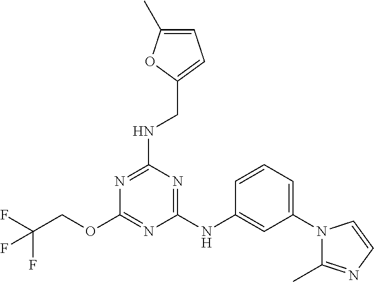 | N-[(5-methylfuran-2-yl)methyl]-N′-[3- (2-methyl-1H-imidazol-1-yl)phenyl]-6- (2,2,2-trifluoroethoxy)-1,3,5-triazine- 2,4-diamine | 459.4 | C21H20F3N7O2 |
| 9 | 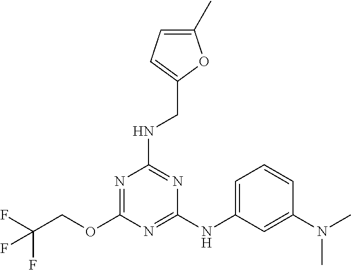 | N-[3-(dimethylamino)phenyl]-N′-[(5- methylfuran-2-yl)methyl]-6-(2,2,2- trifiuoroethoxy)-1,3,5-triazine-2,4- diamine | 422.4 | C19H21F3N6O2 |
| 10 | 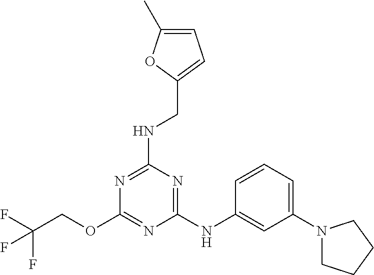 | N-[(5-methylfuran-2-yl)methyl]-N′-[3- (pyrrolidin-1-yl)phenyl]-6-(2,2,2- trifluoroethoxy)-1,3,5-triazine-2,4- diamine | 448.4 | C21H23F3N6O2 |
| 11 | 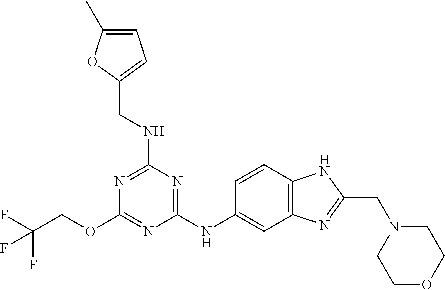 | N-[(5-methylfuran-2-yl)methyl]-N′-[2- (morpholin-4-ylmethyl)-1H- benzimidazol-6-yl]-6-(2,2,2- trifluoroethoxy)-1,3,5-triazine-2,4- diamine | 518.5 | C23H25F3N8O3 |
| 12 | 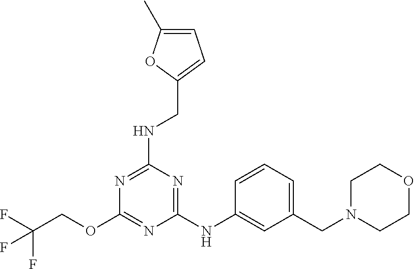 | N-[(5-methylfuran-2-yl)methyl]-N′-[3- (morpholin-4-ylmethyl)phenyl]-6- (2,2,2-trifluoroethoxy)-1,3,5-triazine- 2,4-diamine | 478.5 | C22H25F3N6O3 |
| 13 | 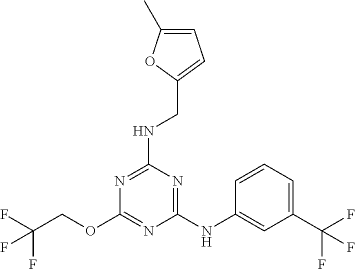 | N-[(5-methylfuran-2-yl)methyl]-6- (2,2,2-trifluoroethoxy)-N′-[3- (trifluoromethyl)phenyl]-1,3,5-triazine- 2,4-diamine | 447.3 | C18H15F6N5O2 |
| 14 | 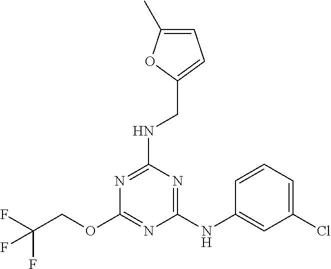 | N-(3-chlorophenyl)-N′-[(5-methylfuran- 2-yl)methyl]-6-(2,2,2-trifluoroethoxy)- 1,3,5-triazine-2,4-diamine | 413.8 | C17H15ClF3N5O2 |
| 15 | 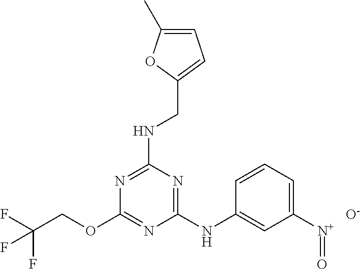 | N-[(5-methylfuran-2-yl)methyl]-N′-(3- nitrophenyl)-6-(2,2,2-trifluoroethoxy)- 1,3,5-triazine-2,4-diamine | 424.3 | C17H15F3N6O4 |
| 16 | 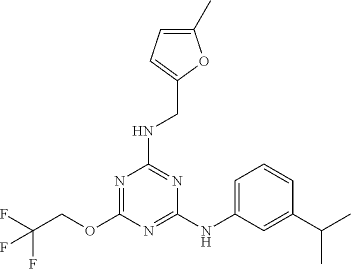 | N-[(5-methylfuran-2-yl)methyl]-N′-[3- (propan-2-yl)phenyl]-6-(2,2,2- trifluoroethoxy)-1,3,5-triazine-2,4- diamine | 421.4 | C20H22F3N5O2 |
| 17 | 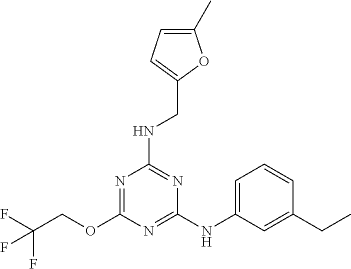 | N-(3-ethylphenyl)-N′-[(5-methylfuran- 2-yl)methyl]-6-(2,2,2-trifluoroethoxy)- 1,3,5-triazine-2,4-diamine | 407.4 | C19H20F3N5O2 |
| 18 | 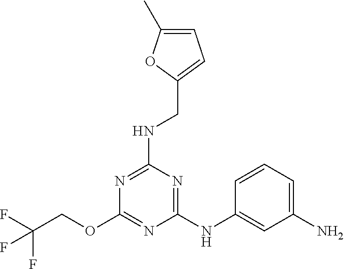 | N-(3-aminophenyl)-N′-(5-methylfuran- 2-yl)methyl]-6-(2,2,2-trifluoroethoxy}- 1,3,5-triazine-2,4-diamine | 394.4 | C17H17F3N6O2 |

### Procedures and Analytical Data for Compounds in Table 11.

### 1. 5-[4-[(5-Methyl-furan-2-ylmethyl)-amino]-6-(2,2,2-trifluoro-ethoxy)-[1,3,5]triazin-2-ylamino]-1,3-dihydro-benzoimidazol-2-one

To a solution of (4,6-Dichloro-[1,3,5]triazin-2-yl)-(5-methyl-furan-2-ylmethyl)-amine (777 mg, 3.0 mmol) in DMSO (2.5 mL) a solution of 5-amino-1,3-dihydro-benzoimidazol-2-one (447 mg, 3.0 mmol) and DIPEA (387 mg, 3.0 mmol) in DMSO (2.5 mL) was added at room temperature. The obtained mixture was stirred at room temperature for 2 hours, diluted with water. The formed solid was collected by filtration and purified by column chromatography (silica gel ethyl acetate) to give 5-{4-Chloro-6-[(5-methyl-furan-2-ylmethylyamino-]-[1,3,5]triazin-2-ylamino}-1,3-dihydro-benzoimidazol-2-one (600 mg, 53%). Sodium hydride (60% in oil, 117 mg 3.0 mmol) was added slowly to a solution of 2,2,2-trifluoroethanol (260 mg, 2.6 mmol) in DMF (5 mL) at 0° C. The obtained mixture was allowed to warm up to room temperature. The formed intermediate (500 mg, 1.3 mmol) was added to the mixture at room temperature and the resulting mixture was stirred at room temperature for 4 hours, diluted with water. The formed solid was collected by filtration and purified by recrystallization from ethanol, prepTLC (10% MeOH/ethyl acetate), recrystallization from ethanol and from MeOH/ether giving the desired compound (yield 93 mg, 16%). ¹H-NMR (400 MHz, DMSO-D₆) δ_{H}: 2.22 (3H, s), 4.42 (2H, broad), 4.93 (2H, broad), 5.96 (1H, broad), 6.12 (1H, broad), 6.80 (1H, d, J=8.5 Hz), 7.20-7.45 (1H, broad, Z/E forms), 7.22 (1H, broad), 7.89 (1H, broad), 9.29-9.51 (1H, broad, Z/E forms), 10.28-10.53 (2H, broad, Z/E forms). LCMS t_{R} (min): 1.66. MS (APCI), m/z 436.04 [M+H]⁺. HPLC t_{R} (min): 11.72. Mₚ 164-166° C.

### 2. N-(5-Methyl-furan-2-ylmethyl)-N′-(3-morpholin-4-yl-phenyl)-6-(2,2,2-trifluoro-ethoxy)-[1,3,5]triazine-2,4-diamine

A mixture of [4-Chloro-6-(2,2,2-trifluoro-ethoxy)-[1,3,5]triazin-2-yl]-(5-methyl-furan-2-ylmethyl)-amine (I-13) (300 mg, 0.93 mmol), 3-Morpholin-4-yl-phenylamine (200 mg, 1.12 mmol), K₂CO₃ (500 mg, 3.62 mmol) and DMSO (3 mL) was stirred at 90° C. for 3 hours, cooled to room temperature, diluted with water, extracted with chloroform. The combined organic phases were washed with water, dried over sodium sulfate and concentrated. Purification by column chromatography (silica gel, chloroform/acetone) gave the desired compound (85 mg, 20%). ¹H-NMR (400 MHz, DMSO-D₆) δ_{H}: 2.22 (3H, s), 3.00-3.15 (4H, broad, Z/E forms), 3.65-3.80 (4H, broad, Z/E forms), 4.40-4.55 (2H, broad, Z/E forms), 4.98 (2H, broad), 5.98 (1H, broad), 6.12 (1H, broad), 6.62 (1H, broad), 7.05-7.25 (1H, broad, Z/E forms), 7.12 (1H, broad), 7.29-7.55 (1H, broad, Z/E forms), 8.00 (1H, broad), 9.31-9.51 (1H, broad, Z/E forms). LCMS t_{R} (min): 1.98. MS (APCI), m/z 465.08 [M+H]⁺. HPLC t_{R} (min): 14.33. Mₚ 130-134° C.

### 3. (4-Benzoimidazol-1-yl-6-ethoxy-[1,3,5]triazin-2-yl)-furan-2-ylmethyl-amine

¹H-NMR (400 MHz, DMSO-D₆) δ_{H}: 1.37 (3H, t, J=7.5 Hz), 4.40-4.55 (2H, two q, J=7.5 Hz, Z/E forms), 4.45-4.60 (2H, two d, J=7.5 Hz, Z/E forms), 6.32-6.45 (2H, broad, Z/E forms), 7.35 (1H, d, J=8.5 Hz), 7.40 (1H, broad), 7.58 (1H, s), 7.75 (1H, d, J=8.5 Hz), 8.40-8.65 (1H, two d, J=8.5 Hz, Z/E forms), 8.65-8.85 (1H, broad, Z/E forms), 8.95-9.10 (1H, two s, Z/E forms). LCMS t_{R} (min): 1.86. MS (APCI), m/z 337.12 [M+H]⁺. HPLC t_{R} (min): 13.39. M_{P} 200-202° C.

### 4. N-(4-Fluoro-3-trifluoromethyl-phenyl)-N′-(5-methyl-furan-2-ylmethyl)-6-(2,2,2-trifluoro-ethoxy)-[1,3,5]triazine-2,4-diamine

¹H-NMR (400 MHz, DMSO-D₆) δ_{H}: 2.20 (3H, s), 4.42 (2H, s), 4.94 (2H, broad), 5.96 (1H, broad), 6.05-6.14 (1H, broad, Z/E forms), 7.40 (1H, broad), 7.87-8.05 (1H, broad, Z/E forms), 8.10 (1H, broad), 8.10-8.40 (1H, broad, Z/E forms), 9.90 (1H, broad). LCMS t_{R} (min): 2.28. MS (APCI), m/z 465.95 [M+H]⁺. HPLC t_{R} (min): 17.50. M_{P} 151-153° C.

### 5. N-(1H-Indazol-6-yl)-N′-(5-methyl-furan-2-ylmethyl)-6-(2,2,2-trifluoro-ethoxy)-[1,3,5]triazine-2,4-diamine

¹H-NMR (400 MHz, DMSO-D₆) δ_{H}: 2.20 (3H, s), 4.40-4.58 (2H, broad, Z/E forms), 4.94 (2H, broad), 5.96 (1H, broad), 6.18 (1H, broad), 7.39 (1H, broad), 7.63 (1H, d, J=8.5 Hz), 7.93 (1H, s), 7.95 (1H, broad), 7.97-8.15 (1H, broad, Z/E forms), 9.60-9.80 (1H, broad, Z/E forms), 12.24-12.85 (1H, broad, Z/E). LCMS t_{R} (min): 1.95. MS (APCI), m/z 420.02 [M+H]⁺. HPLC t_{R} (min): 13.93. M_{P} 206-208° C.

### 6. N-(3,5-Bis-trifluoromethyl-phenyl)-N′-(5-methyl-furan-2-ylmethyl)-6-(2,2,2-trifluoro-ethoxy)-[1,3,5]triazine-2,4-diamine

¹H-NMR (400 MHz, DMSO-D₆) δ_{H}: 2.13-2.27 (3H, two s, Z/E forms), 4.48 (2H, broad), 4.49 (2H, two q, J=7.5 Hz, Z/E forms), 5.92-6.00 (1H, broad, Z/E forms), 6.07-6.18 (1H, broad, Z/E forms), 7.59-7.67 (1H, broad, Z/E forms), 8.30 (1H, broad), 8.42 (1H, s), 8.49 (1H, s), 10.12-10.32 (1H, broad, Z/E forms). LCMS t_{R} (min): 2.36. MS (APCI), m/z 515.95 [M+H]⁺. HPLC t_{R} (min): 18.66. M_{P} 151-153° C.

### 7. N-(5-Methyl-furan-2-ylmethyl)-N′-[3-(2-methyl-imidazol-1-ylmethyl)-phenyl]-6-(2,2,2-trifluoro-ethoxy)-[1,3,5]triazine-2,4-diamine

¹H-NMR (400 MHz, DMSO-D₆) δ_{H}: 2.20 (6H, two s), 4.42 (2H, broad), 4.84-5.04 (2H, two q, J=7.5 Hz), 5.10 (2H, d, J=7.5 Hz), 5.96 (1H, broad), 6.07-6.20 (1H, broad, Z/E forms), 6.74 (2H, broad), 7.01-7.09 (1H, broad, Z/E forms), 7.25 (1H, superposition of two t, J=8.5 Hz), 7.52 (1H, broad), 7.62 (1H, broad d, J=8.5 Hz), 7.90-8.05 (1H, broad, Z/E forms), 9.53-9.69 (1H, broad, Z/E forms). LCMS t_{R} (min): 1.57. MS (APCI), m/z 474.12 [M+H]⁺. HPLC t_{R} (min): 11.31.

### 8. N-(5-Methyl-furan-2-ylmethyl)-N′-[3-(2-methyl-imidazol-1-yl)-phenyl]-6-(2,2,2-trifluoro-ethoxy)-[1,3,5]triazine-2,4-diamine

¹H-NMR (400 MHz, DMSO-D₆) δ_{H}: 2.18-2.20 (3H, two s, Z/E forms), 2.28-2.32 (3H, two s. Z/E forms), 4.44 (2H, broad d, J=7.5 Hz), 4.97 (2H, superposition of two broad q, J=7.5 Hz, Z/E forms), 5.92-5.95 (1H, two broad signals, Z/E forms), 5.95-6.12 (1H, two broad signals. Z/E forms), 6.88 (1H, broad d, J=8.5 Hz, Z/E forms), 7.03 (1H, broad t, J=8.5 Hz, Z/E forms), 7.22 (1H, d, J=8.5 Hz), 7.42 (1H, broad d, J=8.5 Hz, Z/E forms), 7.78 (1H, broad), 7.86 (1H, broad), 8.13 (1H, broad), 9.75-9.88 (1H, two broad signals. Z/E forms). MW 459.43. LCMS t_{R} (min): 1.1.57. MS (APCI), m/z 460.24 [M+H]⁺. HPLC t_{R} (min): 11.35. M_{P} 108-110° C.

### 9. N-(3-Dimethylamino-phenyl)-N′-(5-methyl-furan-2-ylmethyl)-6-(2,2,2-trifluoro-ethoxy)-[1,3,5]triazine-2,4-diamine

¹H-NMR (400 MHz, DMSO-D₆) δ_{H}: 2.20 (3H, s), 2.84 (6H, broad), 4.37-4.49 (2H, broad, Z/E forms), 4.93 (2H, broad), 5.94 (1H, broad), 6.09 (1H, broad), 6.38 (1H, d, J=8.5 Hz), 6.91-7.10 (1H, broad, Z/E forms), 7.06 (1H, broad), 7.06-7.28 (1H, broad, Z/E forms), 7.92 (1H, broad), 9.20-9.44 (1H, broad, Z/E forms). LCMS t_{R} (min): 2.44. MS (APCI), m/z 423.04 [M+H]⁺. HPLC t_{R} (min): 12.00. M_{P} 136-138° C.

### 10. N-(5-Methyl-furan-2-ylmethyl)-N′-(3-pyrrolidin-1-yl-phenyl)-6-(2,2,2-trifluoro-ethoxy)[1,3,5]triazine-2,4-diamine

To a mixture of 3-pyrrolidin-1-yl-phenylamine (330 mg, 2 mmol) and Et₃N (0.3 mL) in dry acetone (7 mL) cyanuric chloride (368 mg, 2 mmol) was added at −10° C. The mixture was stirred at 0° C. for 30 minutes, diluted with aqueous solution of NaHCO₃ and extracted with dichloromethane. The combined organic phases were dried over sodium sulfate, concentrated, purified by column chromatography (silica gel, dichloromethane) and triturated with hexane to give (4,6-Dichloro-[1,3,5]triazin-2-yl)-(3-pyrrolidin-1-yl-phenyl)-amine. Yield 287 mg, 46%.

To a solution of the dichlorotriazine intermediate (200 mg, 0.64 mmol) in acetonitrile (3 mL) a solution of 5-methylfurfuryl amine (67 mg, 0.61 mmol) and Et₃N (0.1 ml) acetonitrile (2 mL) was added at 0° C. The mixture was stirred at room temperature for 30 minutes and diluted with water. The formed precipitate was collected by filtration, washed with water and dried to give 6-Chloro-N-(5-methyl-furan-2-ylmethyl)-N′-(3-pyrrolidin-1-yl-phenyl)-[1,3,5]triazine-2,4-diamine (140 mg, 60%).

To a suspension of sodium hydride (60% in oil, 100 mg) in dry THF (3 mL) 2,2,2-trifluoroethanol (2 mL) was added. The mixture was stirred for 10 minutes at room temperature and added to a solution of the monochlorotriazine intermediate (140 mg, 0.36 mmol) in THF (2 mL). The resulting mixture was stirred at refluxing for 3 hours and diluted with water. The formed solid was collected by filtration, washed with water and hexane and dried to give the final compound. Yield 140 mg, 86%. ¹H-NMR (400 MHz, DMSO-D₆) δ_{H}: 1.91 (4H, broad), 2.21 (3H, s), 3.12 (4H, broad), 4.38-4.54 (2H, two broad signals, Z/E forms), 4.96 (2H, superposition of two broad q, J=7.5 Hz, Z/E forms), 5.94 (1H, s), 6.10 (1H, s), 6.20 (1H, d, J=8.5 Hz), 6.84-6.97 (1H, two broad signals, Z/E forms), 7.02 (1H, t, J=8.5 Hz), 7.09-7.18 (1H, broad, Z/E forms), 7.94 (1H, broad), 9.20-9.45 (1H, broad, Z/E forms). MW 448.45. LCMS t_{R} (min): 2.13. MS (APCI), m/z 449.11 [M+H]⁺. HPLC t_{R} (min): 15.72. M_{P} 145-14° C.

### 11. N-(5-Methyl-furan-2-ylmethyl)-N′-(2-morpholin-4-ylmethyl-3H-benzoimidazol-5-yl)-6-(2,2,2-trifluoro-ethoxy)-[1,3,5]triazine-2,4-diamine

¹H-NMR (400 MHz, DMSO-D₆) δ_{H}: 2.22 (3H, s), 2.45 (4H, m), 3.61 (4H, m), 3.70 (2H, s), 4.42-4.51 (2H, two broad signals, Z/E forms), 4.95 (2H, broad), 5.92 (1H, broad), 6.10-6.18 (1H, broad, Z/E forms), 7.31 (1H, broad), 7.41 (1H, broad), 7.81-7.91 (1H, broad, Z/E forms), 7.91-8.03 (1H, broad, Z/E forms), 9.61-9.38 (1H, broad, Z/E forms), 12.10-12.18 (1H, broad, Z/E forms). MW 518.50. LCMS t_{R} (min): 1.57. MS (APCI) m/z 519.06 [M+H]⁺. HPLC t_{R} (min) 10.67. M_{P} 130-132° C.

### 12. N-(5-Methyl-furan-2-ylmethyl)-N′-(3-morpholin-4-ylmethyl-phenyl)-6-(2,2,2-trifluoro-ethoxy)-[1,3,5]triazine-2,4-diamine

¹H-NMR (400 MHz, DMSO-D₆) δ_{H}: 2.20 (3H, s), 2.32 (4H, m), 3.32-3.47 (2H, broad, Z/E forms), 3.56 (4H, broad), 4.38-4.50 (2H, broad, Z/E forms), 4.95 (2H, superposition of two q, J=7.5 Hz, Z/E forms), 5.95 (1H, broad), 6.12 (1H, d, J=3.6 Hz), 6.93 (1H, d, J=8.5 Hz), 7.20 (1H, t, J=8.5 Hz), 7.46-7.61 (1H, two broad d, J=8.5 Hz, Z/E forms), 7.61-7.89 (1H, broad, Z/E forms), 8.00 (1H, broad), 9.40-9.58 (1H, broad, Z/E forms). MW 478.48. LCMS t_{R} (min): 1.59. MS (APCI), m/z 479.14 [M+H]⁺. HPLC t_{R} (min): 11.00. M_{P} 101-103° C.

### 13. N-(5-Methyl-furan-2-ylmethyl)-6-(2,2,2-trifluoro-ethoxy)-N′-(3-trifluoromethyl-phenyl)-[1,3,5]triazine-2,4-diamine

Sodium (72 mg, 3.13 mmol) was dissolved in 2,2,2-trifluoroethanol (7) (2.5 mL) at room temperature. The obtained solution was added to a solution of 6-Chloro-N-(4-chloro-phenyl)-N′-furan-2-ylmethyl-[1,3,5]triazine-2,4-diamine (I-16) (300 mg, 0.78 mmol) in 2,2,2-trifluoroethanol (7) (1 mL). The resulting mixture as stirred at 100° C. for 4 hours, cooled to room temperature and concentrated at reduced pressure. The residue was purified by prepTLC (30% ethyl acetate/hexane) giving final compound.

Yield 207 mg, 60%. ¹H-NMR (400 MHz, DMSO-D₆) δ_{H}: 2.12-2.25 (3H, two s, Z/E forms), 4.44 (2H, broad), 4.97 (2H, superposition of two q, J=7.5 Hz, Z/E forms), 5.94 (1H, broad), 6.04-6.16 (1H, broad, Z/E forms), 7.31 (1H, broad t, J=8.5 Hz), 7.49 (1H, superposition of two d, J=8.5 Hz, Z/E forms), 7.84-8.05 (1H, two broad d, J=8.5 Hz, Z/E forms), 8.07-8.16 (1H, broad, Z/E forms), 8.11-8.31 (1H, broad, Z/E forms), 9.78-10.00 (1H, broad, Z/E forms). MW 447.34. LCMS t_{R} (min): 2.10. MS (APCI), m/z 448.00 [M+H]⁺. HPLC t_{R} (min): 16.79. M_{P} 72-74° C.

### 14. N-(3-Chloro-phenyl)-N′-(5-methyl-furan-2-ylmethyl)-6-(2,2,2-trifluoro-ethoxy)-[1,3,5]triazine-2,4-diamine

A solution of m-chloro aniline (254 mg, 2 mmol) and DIPEA (258 mg, 2 mmol) in THF (10 mL) was added to a solution of compound I-6 (518 mg, 2 mmol) in THF (10 mL). The mixture was stirred at room temperature for 96 hours (TLC control) and washed with water. The aqueous layer was separated and extracted with ethyl acetate. The combined organic phases were dried over sodium sulfate and concentrated at reduced pressure. Purification by column chromatography (silica gel, acetone/dichloromethane) and recrystallization from ethyl acetate/hexane gave compound 6-Chloro-N-(3-chloro-phenyl)-N′-(5-methyl-furan-2-ylmethyl)-[1,3,5]-triazine-2,4-diamine. Yield 382 mg, 54%.

### N-(3-Chloro-phenyl)-N′-(5-methyl-furan-2-ylmethyl)-6-(2,2,2-trifluoro-ethoxy)-[1,3,5]triazine-2,4-diamine

To a solution of 2,2,2-trifluoroethanol (600 mg, 6 mmol) in THF (5 mL) sodium (69 mg, 3 mmol) was added at room temperature. After dissolving the resulting solution was stirred at room temperature for 10 minutes. To the obtained solution 6-Chloro-N-(3-chloro-phenyl)-N′-(5-methyl-furan-2-ylmethyl)-[1,3,5]triazine-2,4-diamine (250 mg, 1 mmol) was added. The reaction mixture was stirred at room temperature for 2 hours, diluted with water and extracted with ethyl acetate. Purification by column chromatography (silica gel, 3% acetone/dichloromethane) and by recrystallization from diethyl ether/hexane furnished compound (265 mg, 64%). ¹H-NMR (400 MHz, DMSO-D₆) δ_{H}: 2.20 (3H, s), 4.44 (2H, broad), 4.97 (2H, m), 5.94 (1H, broad), 6.12 (1H, broad), 7.02 (1H, broad t, J=8.5 Hz), 7.28 (1H, superposition of two d, J=8.5 Hz), 7.55-7.67 (1H, two broad d, J=8.5 Hz, Z/E forms), 7.92 (1H, superposition of two s), 8.14 (1H, broad), 9.67-9.88 (1H, broad, Z/E forms). MW 413.79. LCMS t_{R} (min): 2.08. MS (APCI), m/z 413.97 [M+H]⁺. HPLC t_{R} (min): 16.54. M_{P} 130-132° C.

### 15. N-(5-Methyl-furan-2-ylmethyl)-N′-(3-nitro-phenyl)-6-(2,2,2-trifluoro-ethoxy)-[1,3,5]triazine-2,4-diamine

¹H-NMR (400 MHz, DMSO-D₆) δ_{H}: 2.15-2.25 (3H, two s, Z/E forms), 4.40-4.53 (2H, two broad signals, Z/E forms), 4.98 (2H, superposition of two q, J=7.5 Hz, Z/E forms), 5.95 (1H, broad, Z/E forms), 6.12 (1H, broad, Z/E forms), 7.55 (1H, superposition of two d, J=8.5 Hz, Z/E forms), 7.82 (1H, broad t, J=8.5 Hz, Z/E forms), 8.02-8.17 (1H, two d, J=8.5 Hz, Z/E forms), 8.19 (1H, broad, Z/E forms), 8.64-8.88 (1H, two broad signals, Z/E forms), 10.00-10.15 (1H, two broad signals, Z/E forms). MW 424.34. LCMS t_{R} (min): 2.09. MS (APCI) m/z 425.01 [M+H]⁺. HPLC t_{R} (min) 15.65. M_{P} 73-75° C.

### 16. N-(3-Isopropyl-phenyl)-N′-(5-methyl-furan-2-ylmethyl)-6-(2,2,2-trifluoro-ethoxy)-[1,3,5]triazine-2,4-diamine

¹H-NMR (400 MHz, DMSO-D₆) δ_{H}: 1.19 (6H, superposition of two d, J=7.5 Hz, Z/E forms), 2.20 (3H, s), 2.81 (1H, m), 4.40-4.48 (2H, two broad signals, Z/E forms), 4.95 (2H, superposition of two q, Z/E forms), 5.95 (1H, broad, Z/E forms), 6.07-6.12 (1H, two broad, Z/E forms), 6.87 (1H, d, J=8.5 Hz), 7.17 (1H, t, J=8.5 Hz), 7.42-7.54 (1H, two d, J=8.5 Hz, Z/E forms), 7.54-7.68 (1H, two broad signals, Z/E forms), 8.00 (1H, broad, Z/E forms), 9.42-9.58 (1H, two broad signals, Z/E forms). MW 421.43. LCMS t_{R} (min): 2.16. MS (APCI) m/z 422.02 [M+H]⁺. HPLC t_{R} (min) 17.39. M_{P} 106-108° C.

### 17. N-(3-Ethyl-phenyl)-N′-(5-methyl-furan-2-ylmethyl)-6-(2,2,2-trifluoro-ethoxy)-[1,3,5]triazine-2,4-diamine

To a solution of cyanuric chloride (500 mg, 2.7 mmol) in THF (5 mL) a solution of 3-ethylaniline (330 mg, 2.7 mmol) and DIPEA (350 mg, 2.7 mmol) in THF (5 mL) was added at −30° C. The mixture was stirred at −30° C. for 2 hours and warmed up to 0° C. Then to the obtained solution a solution of 5-methylfurfurylamine (330 mg, 2.7 mmol) and DIPEA (0.35 mL, 2.7 mmol) in THF (5 mL) was added dropwise at 0° C. The resulting mixture was stirred at 0° C. for 2 hours and at room temperature for 1 hour and concentrated at reduced pressure. The residue was washed with water and a mixture of ethyl acetate/hexane (3/7) and dried giving 6-Chloro-N-(3-ethyl-phenyl)-N′-(5-methyl-furan-2-ylmethyl)-[1,3,5]triazine-2,4-diamine (875 mg, 94% for two steps).

Sodium (50 mg, 2.17 mmol) was dissolved in 2,2,2-trifluoroethanol (22) (0.5 mL) at room temperature. Then the obtained solution was added to a solution of the chloro intermediate (238 mg, 0.7 mmol) in 2,2,2,-trifluorotethanol (1 mL). The resulting mixture was stirred at 100° C. for 5 hours, concentrated at reduced pressure, washed with water and a mixture of ethyl acetate and hexane (1/5) and purified by column chromatography (silica gel, 20% ethyl acetate/hexane) giving thr final compound (30 mg, 11%). ¹H-NMR (400 MHz, DMSO-D₆) δ_{H}: 1.15 (3H, superposition of two t, Z/E forms), 2.20 (3H, s), 2.55 (2H, superposition of two q, Z/E forms), 4.50 (2H, superposition of two d, J=7.5 Hz, Z/E forms), 4.97 (2H, superposition of two q, J=7.5 Hz, Z/E forms), 5.91 (1H, d, J=3.6 Hz), 6.08-6.12 (1H, broad, Z/E forms), 6.85 (1H, d, J=8.5 Hz), 7.15 (1H, broad t, J=8.5 Hz), 7.43-7.53 (1H, broad, Z/E forms), 7.53-7.61 (1H, broad, Z/E forms), 8.00 (1H, broad), 9.40-9.58 (1H, broad, Z/E forms). MW 407.40. LCMS t_{R} (min): 2.08. MS (APCI) m/z 408.05 [M+H]⁺. HPLC t_{R} (min) 16.37. M_{P} 55-57° C.

### 18. N-(3-Amino-phenyl)-N′-(5-methyl-furan-2-ylmethyl)-6-(2,2,2-trifluoro-ethoxy)-[1,3,5]triazine-2,4-diamine

To a solution of compound 15 in the table (150 mg, 0.353 mmol) and hydrazine hydrate (60 mg, 1.081 mmol) in ethanol (3 mL) a suspension of Ra—Ni in water (0.5 mL) was added dropwise at room temperature. The mixture was stirred at room temperature for 3 hours, filtered and concentrated. The residue was purified by column chromatography (silica gel, 30% acetone/dichloromethane) and by prepTLC (20% acetone/dichloromethane) giving the compound (94 mg, 67%). ¹H-NMR (400 MHz, DMSO-D₆) δ_{H}: 2.19 (3H, s), 4.41 (2H, broad), 4.95 (4H, broad), 5.94 (1H, d, J=3.6 Hz), 6.11 (1H, d, J=3.6 Hz), 6.23 (1H, d, J=8.5 Hz), 6.87 (2H, broad), 6.93 (1H, broad), 7.82-7.90 (1H, two broad signals, Z/E forms), 9.21-9.33 (1H, two broad signals, Z/E forms). MW 394.36. LCMS t_{R} (min): 1.68. MS (APCI) m/z 394.97 [M+H]⁺. HPLC t_{R} (min) 10.77. M_{P} 75-77° C.

TABLE 12

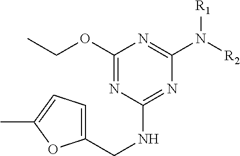

| En | Structure | IUPAC Name | MW | Formula |
|---|---|---|---|---|
| 1 | 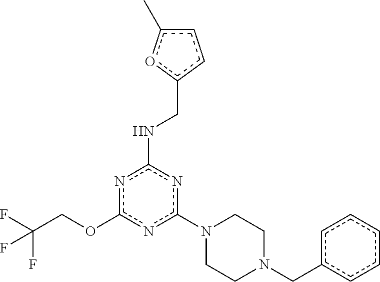 | 4-(4-benzylpiperazin-1-yl)-N-[(5- methylfuran-2-yl)methyl]-6-(2,2,2- trifluoroethoxy)-1,3,5-triazin-2- amine | 462.5 | C22H25F3N6O2 |
| 2 | 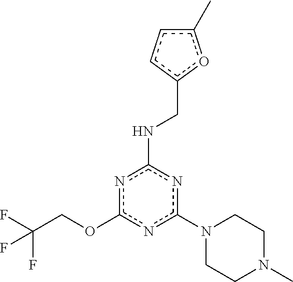 | N-[(5-methylfuran-2-yl)methyl]-4- (4-methylpiperazin-1-yl)-6-(2,2,2- trifluoroethoxy)-1,3,5-triazin-2- amine | 386.4 | C16H21F3N6O2 |
| 3 | 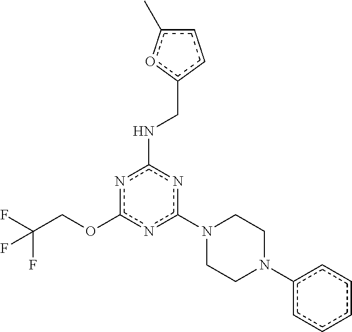 | N-[(5-methylfuran-2-yl)methyl]-4- (4-phenylpiperazin-1-yl)-6-(2,2,2- tnfluoroethoxy)-1,3,5-triazin-2- amine | 448.4 | C21H23F3N6O2 |
| 4 | 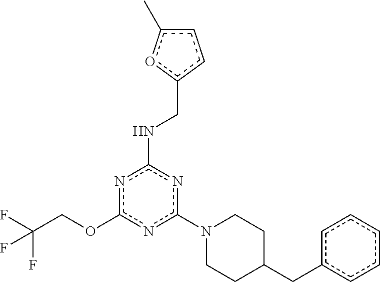 | 4-(4-benzylpiperidin-1-yl)-N-[(5- methylfuran-2-yl)methyl]-6-(2,2,2- trifluoroethoxy)-1,3,5-triazin-2- amine | 461.5 | C23H26F3N5O2 |
| 5 | 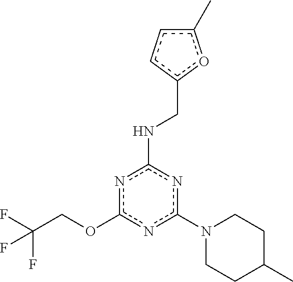 | N-[(5-methylfuran-2-yl)methyl]-4- (4-methylpiperidin-1-yl)-6-(2,2,2- trifluoroethoxy)-1,3,5-triazin-2- amine | 385.4 | C17H22F3N5O2 |
| 6 | 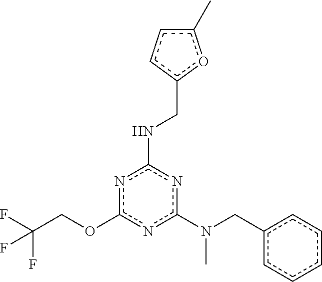 | N-benzyl-N-methyl-N′-[(5- methylfuran-2-yl)methyl]-6-(2,2,2- trifluoroethoxy)-1,3,5-triazine-2,4- diamine | 407.4 | C19H20F3N5O2 |
| 7 | 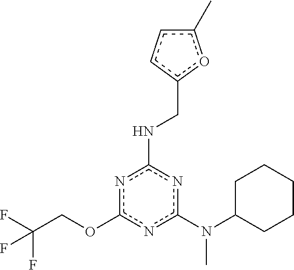 | N-cyclohexyl-N-methyl-N′-[(5- methylfuran-2-yl)methyl]-6-(2,2,2- trifluoroethoxy)-1,3,5-triazine-2,4- diamine | 399.4 | C18H24F3N5O2 |
| 8 | 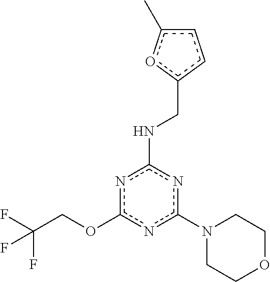 | N-[(5-methylfuran-2-yl)methyl]-4- (morpholin-4-yl)-6-(2,2,2- trifluoroethoxy)-1,3,5-triazin-2- amine | 373.3 | C15H18F3N5O3 |
| 9 | 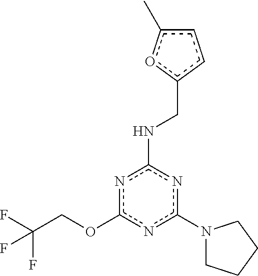 | N-[(5-methylfuran-2-yl)methyl]-4- (pyrrolidin-1-yl)-6-(2,2,2- trifluoroethoxy)-1,3,5-triazin-2- amine | 357.3 | C15H18F3N5O2 |
| 10 | 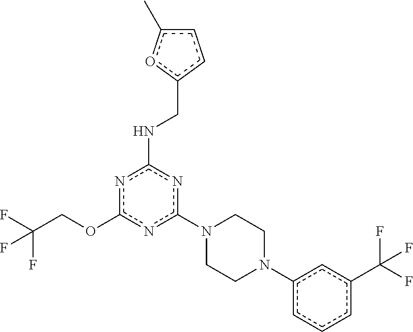 | N-[(5-methylfuran-2-yl)methyl]-4- (2,2,2-trifluoroethoxy)-6-{4-[3- (trifluoromethyl)phenyl]piperazin- 1-yl}-1,3,5-triazin-2-amine | 516.4 | C22H22F6N6O2 |
| 11 | 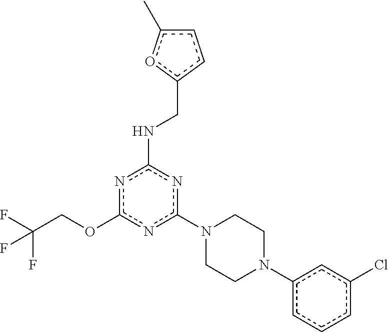 | 4-[4-(3-chlorophenyl)piperazin-1- yl]-N-[(5-methylfuran-2- yl)methyl]-6-(2,2,2- trifluoroethoxy)-1,3,5-triazin-2- amine | 482.9 | C21H22ClF3N6O2 |
| 12 | 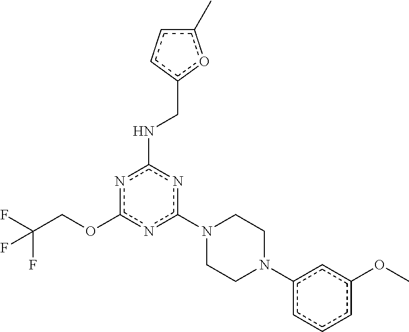 | 4-[4-(3-methoxyphenyl)piperazin- 1-yl]-N-[(5-methylfuran-2- yl)methyl]-6-(2,2,2- trifluoroethoxy)-1,3,5-triazin-2- amine | 478.5 | C22H25F3N6O3 |
| 13 | 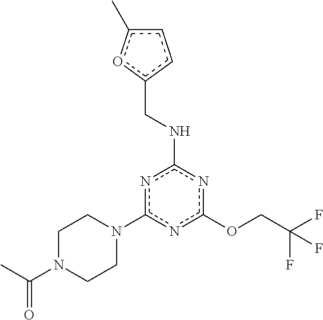 | 1-{4-[4-{[(5-methylfuran-2- yl)methyl]amino}-6-(2,2,2- trifluoroethoxy)-1,3,5-triazin-2- yl]piperazin-1-yl}ethanone | 414.4 | C17H21F3N6O3 |
| 14 | 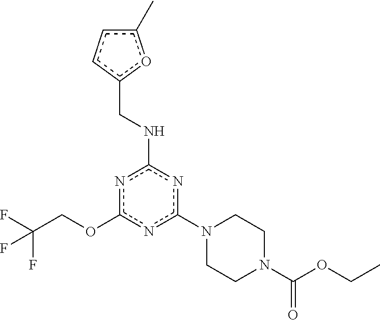 | ethyl 4-[4-{[(5-methylfuran-2- yl)methyl]amino}-6-(2,2,2- trifluoroethoxy)-1,3,5-triazin-2- yl]piperazine-1-carboxylate | 444.4 | C18H23F3N6O4 |
| 15 | 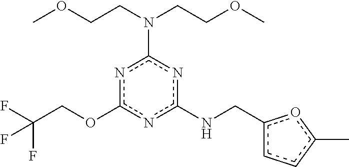 | 1,3,5-triazine-2,4-diamine, N2,N2-bis(2-methoxyethyl)-N4- [(5-methyl-2-furanyl)methyl]-6- (2,2,2-trifluoroethoxy)- | 419.4 | C17H24F3N5O4 |
| 16 | 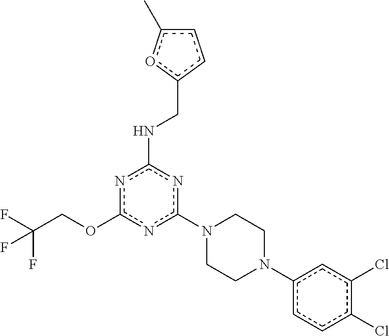 | 4-[4-(3,4- dichlorophenyl)piperazin-1-yl]-N- [(5-methylfuran-2-yl)methyl]-6- (2,2,2-trifluoroethoxy)-1,3,5- triazin-2-amine | 517.3 | C21H21Cl2F3N6O2 |
| 17 | 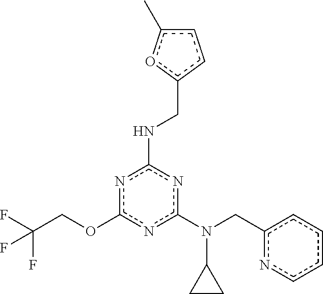 | N-cyclopropyl-N′-[(5-methylfuran- 2-yl)methyl]-N-(pyridin-2- ylmethyl)-6-(2,2,2- trifluoroethoxy)-1,3,5-triazine-2,4- diamine | 434.4 | C20H21F3N6O2 |
| 18 | 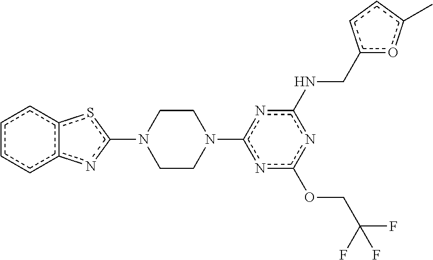 | 4-[4-(1,3-benzothiazol-2- yl)piperazin-1-yl]-N-[(5- methylfuran-2-yl)methyl]-6-(2,2,2- trifluoroethoxy)-1,3,5-triazin-2- amine | 505.5 | C22H22F3N7O2S |
| 19 | 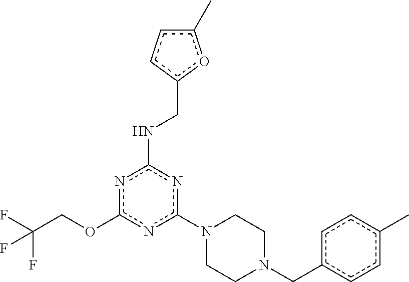 | 4-[4-(4-methylbenzyl)piperazin-1- yl]-N-[(5-methylfuran-2- yl)methyl]-6-(2,2,2- trifluoroethoxy)-1,3,5-triazin-2- amine | 476.5 | C23H27F3N6O2 |
| 20 | 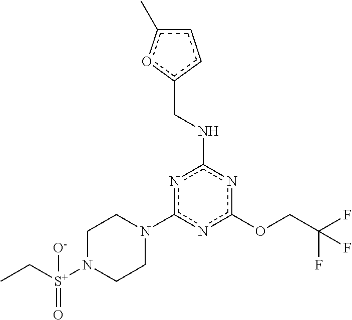 | 4-[4-(ethylsulfonyl)piperazin-1-yl]- N-[(5-methylfuran-2-yl)methyl]-6- (2,2,2-trifluoroethoxy)-1,3,5- triazin-2-amine | 464.5 | C17H23F3N6O4S |
| 21 | 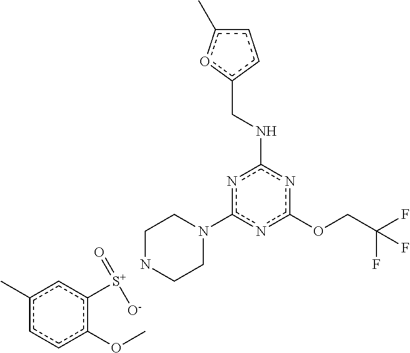 | 4-{4-[(2-methoxy-5- methylphenyl)sulfonyl]piperazin- 1-yl}-N-[(5-methylfuran-2- yl)methyl]-6-(2,2,2- trifluoroethoxy)-1,3,5-triazin-2- amine | 556.6 | C23H27F3N6O5S |
| 22 | 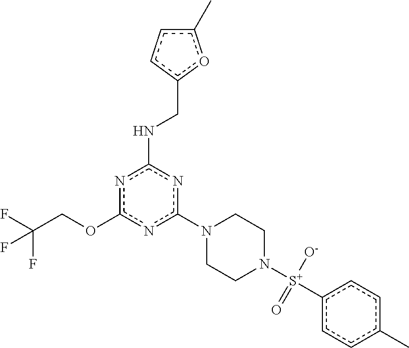 | N-[(5-methylfuran-2-yl)methyl]-4- {4-[(4- methylphenyl)sulfonyl]piperazin- 1-yl}-6-(2,2,2-trifluoroethoxy)- 1,3,5-triazin-2-amine | 526.5 | C22H25F3N6O4S |
| 23 | 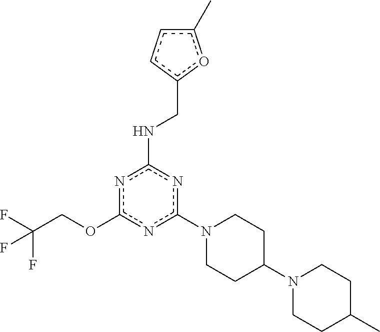 | 4-(4-methyl-1,4″-bipiperidin-1″- yl)-N-[(5-methylfuran-2- yl)methyl]-6-(2,2,2- trifluoroethoxy)-1,3,5-triazin-2- amine | 468.5 | C22H31F3N6O2 |
| 24 | 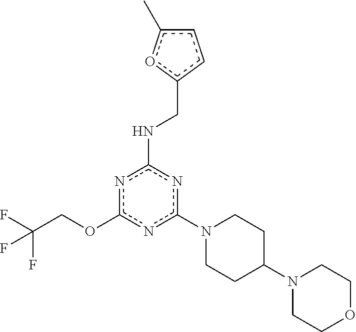 | N-[(5-methylfuran-2-yl)methyl]-4- [4-(morpholin-4-yl)piperidin-1-yl]- 6-(2,2,2-trifluoroethoxy)-1,3,5- triazin-2-amine | 456.5 | C20H27F3N6O3 |
| 25 | 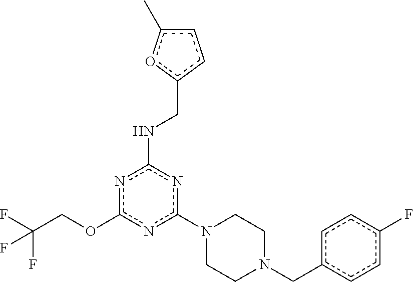 | 4-[4-(4-fluorobenzyl)piperazin-1- yl]-N-[(5-methylfuran-2- yl)methyl]-6-(2,2,2- trifluoroethoxy)-1,3,5-triazin-2- amine | 480.5 | C22H24F4N6O2 |
| 26 | 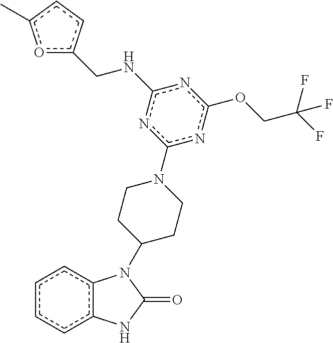 | 1-{1-[4-{[(5-methylfuran-2- yl)methyl]amino}-6-(2,2,2- trifluoroethoxy)-1,3,5-triazin-2- yl]piperidin-4-yl}-1,3-dihydro-2H- benzimidazol-2-one | 503.5 | C23H24F3N7O3 |
| 27 | 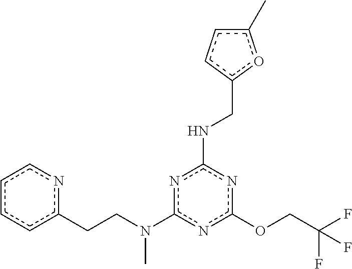 | N-methyl-N′-[(5-methylfuran-2- yl)methyl]-N-[2-(pyridin-2- yl)ethyl]-6-(2,2,2-trifluoroethoxy)- 1,3,5-triazine-2,4-diamine | 422.4 | C19H21F3N6O2 |
| 28 | 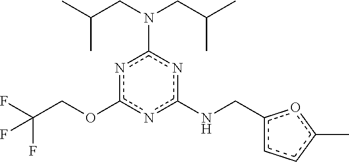 | N″-[(5-methylfuran-2-yl)methyl]- N,N-bis(2-methylpropyl)-6-(2,2,2- trifluoroethoxy)-1,3,5-triazine-2,4- diamine | 415.5 | C19H28F3N5O2 |
| 29 | 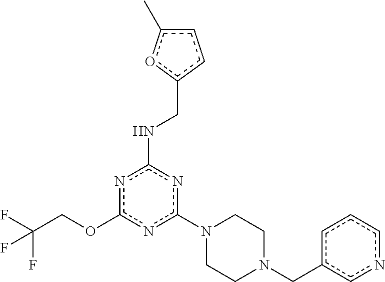 | N-[(5-methylfuran-2-yl)methyl]-4- [4-(pyridin-3-ylmethyl)piperazin- 1-yl]-6-(2,2,2-trifluoroethoxy)- 1,3,5-triazin-2-amine | 463.5 | C21H24F3N7O2 |
| 30 | 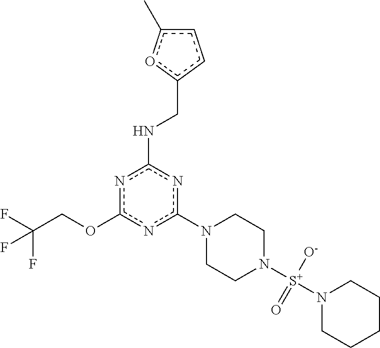 | N-[(5-methylfuran-2-yl)methyl]-4- [4-(piperidin-1- ylsulfonyl)piperazin-1-yl]-6-(2,2,2- trifluoroethoxy)-1,3,5-triazin-2- amine | 519.5 | C20H28F3N7O4S |
| 31 | 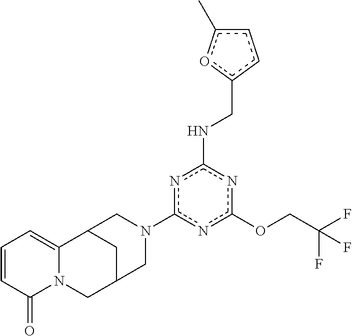 | 3-[4-{[(5-methylfuran-2- yl)methyl]amino}-6-(2,2,2- trifluoroethoxy)-1,3,5-triazin-2-yl]- 1,2,3,4,5,6-hexahydro-8H-1,5- methanopyrido[1,2- a][1,5]diaz^{o}Cin-8-one | 476.5 | C22H23F3N6O3 |
| 32 | 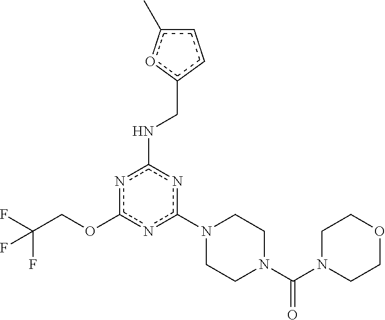 | {4-[4-{[(5-methylfuran-2- yl)methyl]amino}-6-(2,2,2- trifluoroethoxy)-1,3,5-triazin-2- yl]piperazin-1-yl}(morpholin-4- yl)methanone | 485.5 | C20H26F3N7O4 |
| 33 | 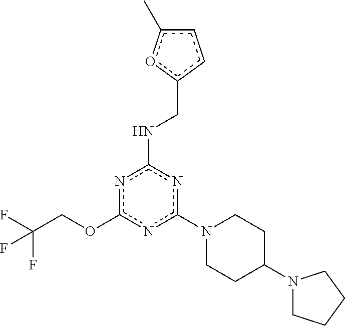 | N-[(5-methylfuran-2-yl)methyl]-4- [4-(pyrrolidin-1-yl)piperidin-1-yl]- 6-(2,2,2-trifluoroethoxy)-1,3,5- triazin-2-amine | 440.5 | C20H27F3N6O2 |
| 34 | 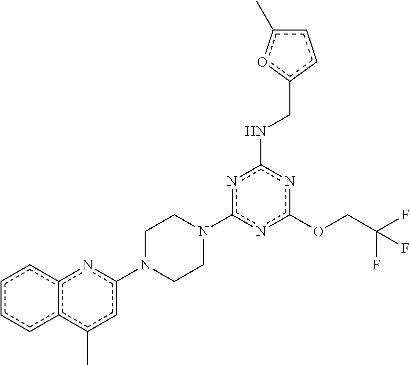 | N-[(5-methylfuran-2-yl)methyl]-4- [4-(4-methylquinolin-2- yl)piperazin-1-yl]-6-(2,2,2- trifluoroethoxy)-1,3,5-triazin-2- amine | 513.5 | C25H26F3N7O2 |
| 35 | 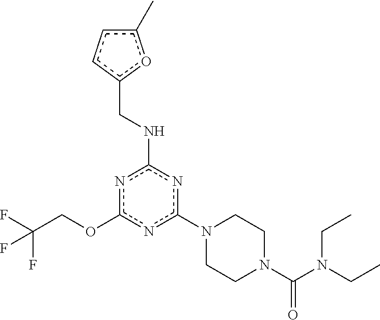 | N,N-diethyl-4-[4-{[(5-methylfuran- 2-yl)methyl]amino}-6-(2,2,2- trifluoroethoxy)-1,3,5-triazin-2- yl]piperazine-1-carboxamide | 471.5 | C20H28F3N7O3 |
| 36 | 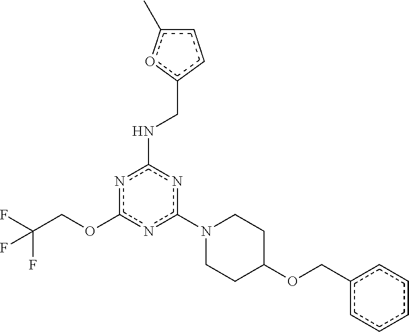 | 4-[4-(benzyloxy)piperidin-1-yl]-N- [(5-methylfuran-2-yl)methyl]-6- (2,2,2-trifluoroethoxy)-1,3,5- triazin-2-amine | 477.5 | C23H26F3N5O3 |
| 37 | 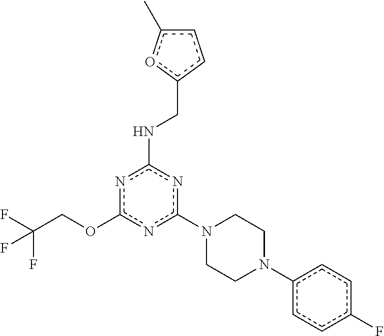 | 4-[4-(4-fluorophenyl)piperazin-1- yl]-N-[(5-methylfuran-2- yl)methyl]-6-(2,2,2- trifluoroethoxy)-1,3,5-triazin-2- amine | 466.4 | C21H22F4N6O2 |
| 38 | 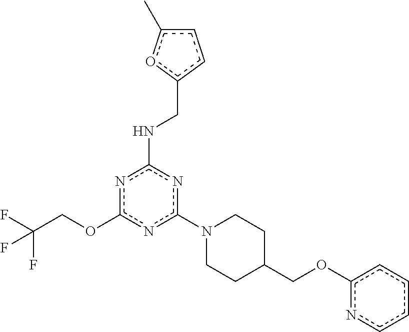 | N-[(5-methylfuran-2-yl)methyl]-4- {4-[(pyridin-2- yloxy)methyl]piperidin-1-yl}-6- (2,2,2-trifluoroethoxy)-1,3,5- triazin-2-amine | 478.5 | C22H25F3N6O3 |
| 39 | 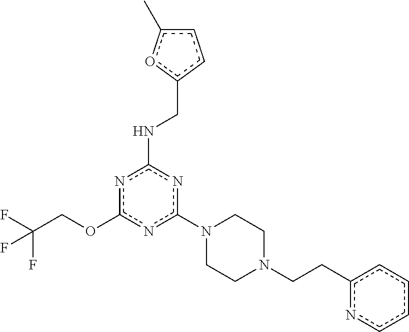 | N-[(5-methylfuran-2-yl)methyl]-4- {4-[2-(pyridin-2-yl)ethyl]piperazin- 1-yl}-6-(2,2,2-trifluoroethoxy)- 1,3,5-triazin-2-amine | 477.5 | C22H26F3N7O2 |
| 40 | 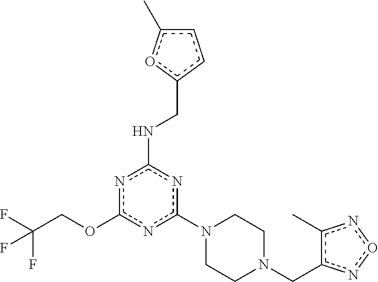 | N-[(5-methylfuran-2-yl)methyl]-4- {4-[(4-methyl-1,2,5-oxadiazol-3- yl)methyl]piperazin-1-yl}-6-(2,2,2- trifluoroethoxy)-1,3,5-triazin-2- amine | 468.4 | C19H23F3N8O3 |
| 41 | 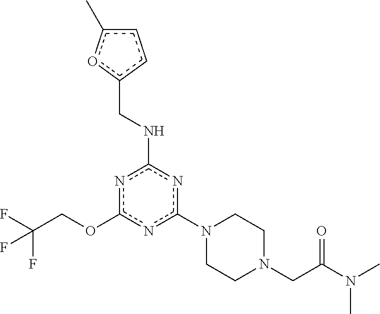 | N,N-dimethyl-2-{4-[4-{[(5- methylfuran-2-yl)methyl]amino}- 6-(2,2,2-trifluoroethoxy)-1,3,5- triazin-2-yl]piperazin-1- yl}acetamide | 457.5 | C19H26F3N7O3 |
| 42 | 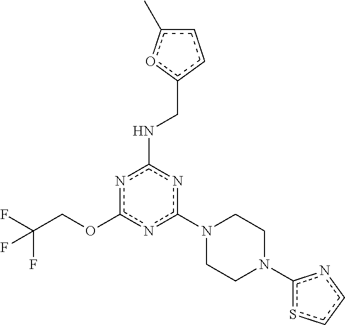 | N-[(5-methylfuran-2-yl)methyl]-4- [4-(1,3-thiazol-2-yl)piperazin-1- yl]-6-(2,2,2-trifluoroethoxy)-1,3,5- triazin-2-amine | 455.5 | C18H20F3N7O2S |
| 43 | 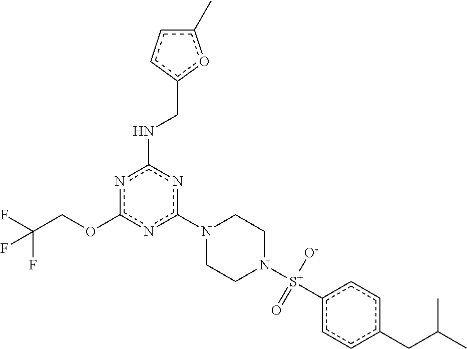 | N-[(5-methylfuran-2-yl)methyl]-4- (4-{[4-(2- methylpropyl)phenyl]sulfonyl} piperazin-1-yl)-6-(2,2,2- trifluoroethoxy)-1,3,5-triazin-2- amine | 568.6 | C25H31F3N6O4S |
| 44 | 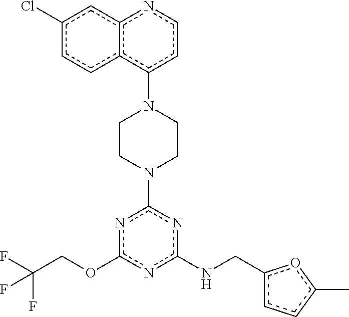 | 4-[4-(7-chloroquinolin-4- yl)piperazin-1-yl]-N-[(5- methylfuran-2-yl)methyl]-6-(2,2,2- trifluoroethoxy)-1,3,5-triazin-2- amine | 533.9 | C24H23ClF3N7O2 |
| 45 | 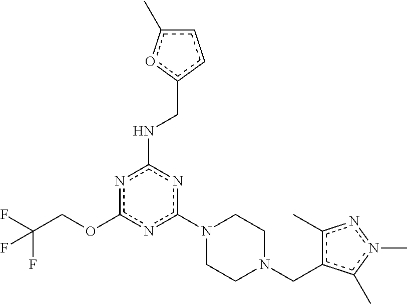 | N-[(5-methylfuran-2-yl)methyl]-4- (2,2,2-trifluoroethoxy)-6-{4- [(1,3,5-trimethyl-1H-pyrazol-4- yl)methyl]piperazin-1-yl}-1,3,5- triazin-2-amine | 494.5 | C22H29F3N8O2 |
| 46 | 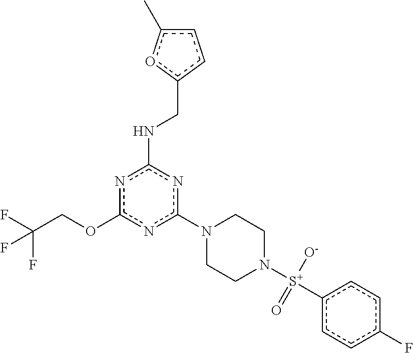 | 4-{4-[(4- fluorophenyl)sulfonyl]piperazin-1- yl}-N-[(5-methylfuran-2- yl)methyl]-6-(2,2,2- trifluoroethoxy)-1,3,5-triazin-2- amine | 530.5 | C21H22F4N6O4S |
| 47 | 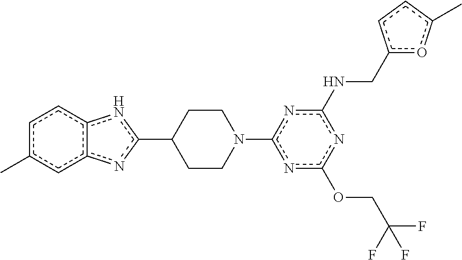 | 4-[4-(5-methyl-1H-benzimidazol- 2-yl)piperidin-1-yl]-N-[(5- methylfuran-2-yl)methyl]-6-(2,2,2- trifluoroethoxy)-1,3,5-triazin-2- amine | 501.5 | C24H26F3N7O2 |
| 48 | 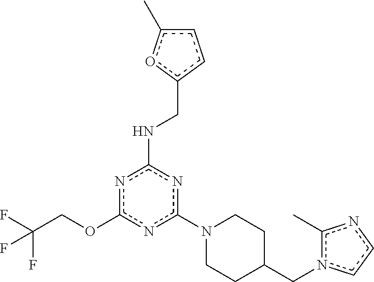 | N-[(5-methylfuran-2-yl)methyl]-4- {4-[(2-methyl-1H-imidazol-1- yl)methyl]piperidin-1-yl}-6-(2,2,2- trifluoroethoxy)-1,3,5-triazin-2- amine | 465.5 | C21H26F3N7O2 |
| 49 | 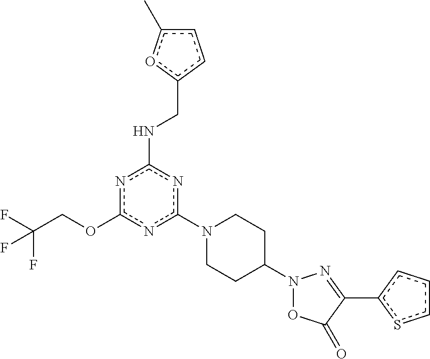 | 2-{1-[4-{[(5-methylfuran-2- yl)methyl]amino}-6-(2,2,2- trifluoroethoxy)-1,3,5-triazin-2- yl]piperidin-4-yl}-4-(thiophen-2- yl)-1,2,3-oxadiazol-5(2H)-one | 537.5 | C22H22F3N7O4S |
| 50 | 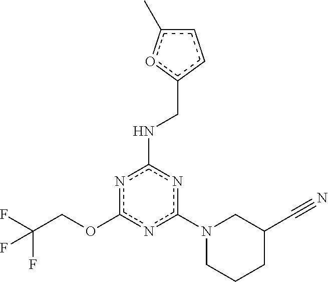 | 1-[4-{[(5-methylfuran-2- yl)methyl]amino}-6-(2,2,2- trifluoroethoxy)-1,3,5-triazin-2- yl]piperidine-3-carbonitrile | 396.4 | C17H19F3N6O2 |
| 51 | 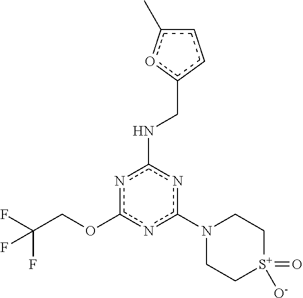 | 4-(1,1-dioxidothiomorpholin-4-yl)- N-[(5-methylfuran-2-yl)methyl]-6- (2,2,2-trifluoroethoxy)-1,3,5- triazin-2-amine | 421.4 | C15H18F3N5O4S |
| 52 | 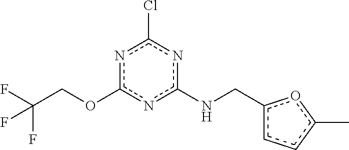 | 4-chloro-N-[(5-methylfuran-2- yl)methyl]-6-(2,2,2- trifluoroethoxy)-1,3,5-triazin-2- amine | 322.7 | C11H10ClF3N4O2 |
| 53 | 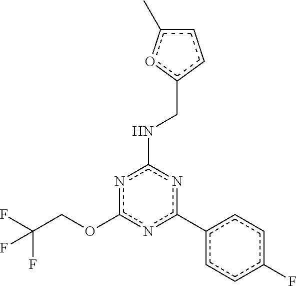 | 4-(4-fluorophenyl)-N-[(5- methylfuran-2-yl)methyl]-6-(2,2,2- trifluoroethoxy)-1,3,5-triazin-2- amine | 382.3 | C17H14F4N4O2 |
| | 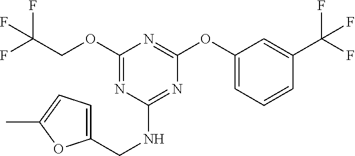 | (5-Methyl-furan-2-ylmethyl)-[4- (2,2,2-trifluoro-ethoxy)-6-(3- trifluoromethyl-phenoxy)- [1,3,5]triazin-2-yl]-amine | 448.10 | C18H14F6N4O3 |

### Procedures and Analytical Data for Compounds in Table 12.

### 1. [4-(4-Benzyl-piperazin-1-yl)-6-(2,2,2-trifluoro-ethoxy)-[1,3,5]triazin-2-yl]-(5-methyl-furan-2-ylmethyl)-amine

Yield 312 mg, 73%. ¹H-NMR (400 MHz, DMSO-D₆) δ_{H}: 2.20 (3H, s), 2.39 (4H, broad), 3.50 (2H, s), 3.70 (4H, broad), 4.36 (2H, broad), 4.90 (2H, superposition of two q, J=7.5 Hz, Z/E forms), 5.92 (1H, d, J=3.6 Hz), 6.09 (1H, d, J=3.6 Hz), 7.30 (5H, m), 7.75 (1H, broad). MW 462.48. LCMS t_{R} (min): 1.60. MS (APCI) m/z 463.13 [M+H]⁺. HPLC t_{R} (min) 11.84. M_{P} 115-117° C.

### 2. (5-Methyl-furan-2-ylmethyl)-[4-(4-methyl-piperazin-1-yl)-6-(2,2,2-trifluoro-ethoxy)-[1,3,5]triazin-2-yl]-amine

Yield 100 mg, 28%. ¹H-NMR (400 MHz, DMSO-D₆) δ_{H}: 2.19 (3H, s), 2.22 (3H, s), 2.30 (4H, broad), 3.70 (4H, broad), 4.38 (2H, d, J=7.5 Hz), 4.89 (2H, superposition of two q, J=7.5 Hz, Z/E forms), 5.94 (1H, d, J=3.6 Hz), 6.09 (1H, broad), 7.74 (1H, broad). MW 386.38. LCMS t_{R} (min): 1.47. MS (APCI) m/z 387.08 [M+H]⁺. HPLC t_{R} (min) 10.10. M_{P} 122-124° C.

### 3. (5-Methyl-furan-2-ylmethyl)-[4-(4-phenyl-piperazin-1-yl)-6-(2,2,2-trifluoro-ethoxy)-[1,3,5]triazin-2-yl]-amine

Yield 372 mg, 89%. ¹H-NMR (400 MHz, DMSO-D₆) δ_{H}: 2.21 (3H, s), 3.13 (4H, m), 3.88 (4H, broad), 4.40 (2H, d, J=7.5 Hz), 4.92 (2H, superposition of two broad q, J=7.5 Hz, Z/E forms), 5.96 (1H, d, J=3.6 Hz), 6.11 (1H, broad), 6.80 (1H, t, J=8.5 Hz), 6.96 (2H, d, J=8.5 Hz), 7.23 (2H, broad t, J=8.5 Hz, Z/E forms), 7.81 (1H, broad t, J=7.5 Hz). MW 448.45. LCMS t_{R} (min): 2.14. MS (APCI), m/z 449.08 [M+H]⁺. HPLC t_{R} (min): 16.15. M_{P} 137-139° C.

### 4. [4-(4-Benzyl-piperidin-1-yl)-6-(2,2,2-trifluoro-ethoxy)-[1,3,5]triazin-2-yl]-(5-methyl-furan-2-ylmethyl)-amine

Yield 202 mg, 57%. ¹H-NMR (400 MHz, DMSO-D₆) δ_{H}: 1.05 (2H, m), 1.60 (2H, m), 1.80 (1H, broad), 2.20 (3H, s), 2.53 (2H, m), 2.80 (2H, broad), 4.35 (2H, broad), 4.47-4.70 (2H, broad, Z/E forms), 4.89 (2H, broad), 5.94 (1H, broad), 6.09 (1H, broad), 7.17 (3H, m), 7.29 (2H, t, J=8.5 Hz), 7.70 (1H, broad triplet, J=7.5 Hz). MW 461.49. LCMS t_{R} (min): 2.30. MS (APCI), m/z 462.11 [M+H]⁺. HPLC t_{R} (min): 18.25. M_{P} 103-105° C.

### 5. (5-Methyl-furan-2-ylmethyl)-[4-(4-methyl-piperidin-1-yl)-6-(2,2,2-trifluoro-ethoxy)-[1,3,5]triazin-2-yl]-amine

Yield 75 mg, 25%. ¹H-NMR (400 MHz, DMSO-D₆) δ_{H}: 0.90 (3H, s), 1.00 (2H, m), 1.64 (3H, m), 2.20 (3H, s), 2.81 (2H, broad), 4.35 (2H, broad), 4.50-4.70 (2H, broad, Z/E forms), 4.90 (2H, broad), 5.95 (1H, broad), 6.08 (1H, broad), 7.70 (1H, broad). MW 385.39. LCMS t_{R} (min): 2.20. MS (APCI) m/z 386.07 [M+H]⁺. HPLC t_{R} (min) 17.27. M_{P} 106-108° C.

### 6. N-Benzyl-N-methyl-N′-(5-methyl-furan-2-ylmethyl)-6-(2,2,2-trifluoro-ethoxy)-[1,3,5]triazine-2,4-diamine

Yield 500 mg, 65%. ¹H-NMR (400 MHz, DMSO-D₆) δ_{H}: 2.2.12-2.22 (3H, two s, Z/E forms), 2.93-3.08 (3H, two s, Z/E forms), 4.40 (2H, broad), 4.72-4.83 (2H, broad, Z/E forms), 4.91 (2H, broad), 5.85-5.98 (1H, broad, Z/E forms), 5.95-6.13 (1H, broad, Z/E forms), 7.24 (3H, m), 7.31 (2H, m), 7.78 (1H, broad). MW 407.40. LCMS t_{R} (min): 2.10. MS (APCI), m/z 408.04 [M+H]⁺. HPLC t_{R} (min): 16.86. M_{P} 45-47° C.

### 7. N-Cyclohexyl-N-methyl-N′-(5-methyl-furan-2-ylmethyl)-6-(2,2,2-trifluoro-ethoxy)-[1,3,5]triazine-2,4-diamine

Yield 400 mg, 50%. ¹H-NMR (400 MHz, DMSO-D₆) δ_{H}: 1.12 (1H, m), 1.32 (2H, m), 1.48 (2H, m), 1.58 (3H, m), 1.78 (2H, m), 2.20 (3H, s), 2.85-3.00 (3H, broad, Z/E forms), 4.39 (2H, broad), 4.50 (1H, broad), 4.89 (2H, broad), 5.95 (1H, broad), 6.08 (1H, broad), 7.70 (1H, broad). MW 399.42. LCMS t_{R} (min): 2.23. MS (APCI), m/z 400.08 [M+H]⁺. HPLC t_{R} (min): 17.72. M_{P} 39-41° C.

### 8. (5-Methyl-furan-2-ylmethyl)[4-morpholin-4-yl-6-(2,2,2-trifluoro-ethoxy)-[1,3,5]triazin-2-yl]-amine

Yield 165 mg, 57%. ¹H-NMR (400 MHz, DMSO-D₆) δ_{H}: 2.21 (3H, s), 3.60 (4H, m), 3.70 (4H, broad), 4.39 (2H, broad d, J=7.5 Hz), 4.90 (2H, superposition of two broad q, J=7.5 Hz, Z/E forms), 5.95 (1H, broad), 6.10 (1H, broad), 7.80 (1H, broad). MW 373.34. LCMS t_{R} (min): 1.87. MS (APCI), m/z 374.04 [M+H]⁺. HPLC t_{R} (min): 14.42. M_{P} 148-150° C.

### 9. (5-Methyl-furan-2-ylmethyl)-[4-pyrrolidin-1-yl-6-(2,2,2-trifluoro-ethoxy)-[1,3,5]triazin-2-yl]-amine

Yield 100 mg, 36%. ¹H-NMR (400 MHz, DMSO-D₆) δ_{H}: 1.88 (4H, broad), 2.20 (3H, s), 3.45 (4H, broad), 4.38 (2H, broad), 4.90 (2H, broad), 5.95 (1H, broad), 6.10 (1H, broad), 7.60-7.76 (1H, broad, Z/E forms). MW 357.34. LCMS t_{R} (min): 2.00. MS (APCI) m/z 358.04 [M+H]⁺. HPLC t_{R} (min) 14.88. M_{P} 150-152° C.

### 10. (5-Methyl-furan-2-ylmethyl)-(4-(2,2,2-trifluoro-ethoxy)-6-[4-(3-trifluoromethyl-phenyl)-piperazin-1-yl]-[1,3,5]triazin-2-yl}-amine

Yield 412 mg, 86%. ¹H-NMR (400 MHz, DMSO-D₆) δ_{H}: 2.20 (3H, s), 3.28 (4H, m), 3.88 (4H, broad), 4.40 (2H, d, J=7.5 Hz), 4.92 (2H, superposition of two q, J=7.5 Hz, Z/E forms), 5.98 (1H, broad), 6.06-6.16 (1H, broad, Z/E forms), 7.08 (1H, d, J=8.5 Hz), 7.19 (1H, s), 7.24 (1H, d, J=8.5 Hz), 7.42 (1H, t, J=8.5 Hz), 7.83 (1H, broad t, J=7.5 Hz). MW 516.45. LCMS t_{R} (min): 2.25. MS (APCI) m/z 517.10 [M+H]⁺. HPLC t_{R} (min) 18.07. M_{P} 148-150° C.

### 11. [4-[4-(3-Chloro-phenyl)-piperazin-1-yl]-6-(2,2,2-trifluoro-ethoxy)-[1,3,5]triazin-2-yl]-(5-methyl-furan-2-ylmethyl)-amine

Yield 120 mg, 27%. ¹H-NMR (400 MHz, DMSO-D₆) δ_{H}: 2.20 (3H, s), 3.22 (4H, m), 3.85 (4H, broad), 4.40 (2H, d, J=7.5 Hz), 4.92 (2H, superposition of two q, J=7.5 Hz, Z/E forms), 5.98 (1H, broad), 6.11 (1H, broad), 6.79 (1H, d, J=8.5 Hz), 6.92 (1H, d, J=8.5 Hz), 6.97 (1H, s), 7.22 (1H, t, J=8.5 Hz), 7.83 (1H, broad). MW 482.90. LCMS t_{R} (min): 2.23. MS (APCI), m/z 483.05, 485.00 [M+H]⁺. HPLC t_{R} (min): 17.82. M_{P} 147-149° C.

### 12. [4-[4-(3-Methoxy-phenyl)-piperazin-1-yl]-6-(2,2,2-trifluoro-ethoxy)-[1,3,5]triazin-2-yl]-(5-methyl-furan-2-ylmethyl)-amine

Yield 197 mg, 44%. ¹H-NMR (400 MHz, DMSO-D₆) δ_{H}: 2.21 (3H, s), 3.16 (4H, m), 3.72 (3H, s), 3.87 (4H, broad), 4.40 (2H, broad), 4.92 (2H, superposition of two broad q, J=7.5 Hz, Z/E forms), 5.95 (1H, d, J=3.6 Hz), 6.10 (1H, broad), 6.39 (1H, d, J=8.5 Hz), 6.48 (1H, s), 6.55 (1H, d, J=8.5 Hz), 7.12 (1H, t, J=8.5 Hz), 7.82 (1H, broad). MW 478.48. LCMS t_{R} (min): 2.11. MS (APCI), m/z 479.10 [M+H]⁺. HPLC t_{R} (min): 16.21. M_{P} 118-120° C.

### 13. 1-{-4-[4-[(5-Methyl-furan-2-ylmethyl)-amino]-6-(2,2,2-trifluoro-ethoxy)-[1,3,5]triazin-2-yl]-piperazin-1-yl}-ethanone

Yield 245 mg, 64%. ¹H-NMR (400 MHz, DMSO-D₆) δ_{H}: 2.03 (3H, s), 2.21 (3H, s), 3.48 (4H, m), 3.62-3.81 (4H, broad, Z/E forms), 4.39 (2H, d, J=7.5 Hz), 4.90 (2H, superposition of two q, J=7.5 Hz, Z/E forms), 5.95 (1H, d, J=3.6 Hz), 6.10 (1H, broad, Z/E forms), 7.82 (1H, broad t, J=7.5 Hz, Z/E forms). MW 414.39. LCMS t_{R} (min): 1.71. MS (APCI) m/z 415.04 [M+H]⁺. HPLC t_{R} (min) 12.90. M_{P} 140-142° C.

### 14. 4-[4-[(5-Methyl-furan-2-ylmethyl)-amino]-6-(2,2,2-trifluoro-ethoxy)-[1,3,5]triazin-2-yl]-piperazine-1-carboxylic acid ethyl ester

Yield 138 mg, 33%. ¹H-NMR (400 MHz, DMSO-D₆) δ_{H}: 1.20 (3H, t, J=7.5 Hz), 2.21 (3H, s), 3.40 (4H, m), 3.72 (4H, broad), 4.08 (2H, broad q, J=7.5 Hz), 4.40 (2H, broad d, J=7.5 Hz), 4.90 (2H, broad q, J=7.5 Hz), 5.95 (1H, d, J=3.6 Hz), 6.06-6.12 (1H, two broad signals, Z/E forms), 7.82 (1H, broad). MW 444.42. LCMS t_{R} (min): 1.95. MS (APCI), m/z 445.09 [M+H]⁺. HPLC t_{R} (min): 15.32. M_{P} 165-167° C.

### 15. N,N-Bis-(2-methoxy-ethyl)-N′-(5-methyl-furan-2-ylmethyl)-6-(2,2,2-trifluoro-ethoxy)-[1,3,5]triazine-2,4-diamine

Yield 83 mg, 32%. ¹H-NMR (400 MHz, DMSO-D₆) δ_{H}: 2.20 (3H, s), 3.25 (6H, s), 3.49 (4H, m), 3.68 (4H, m), 4.35 (2H, d, J=7.5 Hz), 4.90 (2H, superposition of two quartets, J=7.5 Hz), 5.95 (1H, d, J=3.6 Hz), 6.07 (1H, broad), 7.78 (1H, broad). MW 419.41. LCMS t_{R} (min): 1.97. MS (APCI), m/z 420.07 [M+H]⁺. HPLC t_{R} (min): 15.04. M_{P} 31-33° C.

### 16. [4-[4-(3,4-Dichloro-phenyl)-piperazin-1-yl]-6-(2,2,2-trifluoro-ethoxy)-[1,3,5]triazin-2-yl]-(5-methyl-furan-2-ylmethyl)-amine

Yield 173 mg, 36%. ¹H-NMR (400 MHz, DMSO-D₆) δ_{H}: 2.22 (3H, s), 3.25 (4H, m), 3.85 (4H, broad), 4.40 (2H, broad d, J=7.5 Hz), 4.93 (2H, superposition of two q, J=7.5 Hz, Z/E forms), 5.97 (1H, d, J=3.6 Hz), 6.10 (1H, broad), 6.95 (1H, d, J=8.5 Hz), 7.15 (1H, s), 7.40 (1H, d, J=8.5 Hz), 7.83 (1H, broad t, J=7.5 Hz). MW 517.34. LCMS t_{R} (min): 2.31. MS (APCI), m/z 517.03, 519.00 [M+H]⁺. HPLC t_{R} (min): 18.45. M_{P} 159-161° C.

### 17. N-Cyclopropyl-N′-(5-methyl-furan-2-ylmethyl)-N-pyridin-2-ylmethyl-6-(2,2,2-trifluoro-ethoxy)-[1,3,5]triazine-2,4-diamine

Yield 129 mg, 64%. ¹H-NMR (400 MHz, DMSO-D₆) δ_{H}: 0.61-0.81 (4H, broad, Z/E forms), 2.18 (3H, broad), 2.88 (1H, broad), 4.10-4.40 (1H, broad, Z/E forms), 4.30-4.55 (1H, broad, Z/E forms), 4.70-5.00 (2H, broad, Z/E forms), 4.80 (2H, s), 5.80 (1H, broad), 5.90-6.13 (1H, broad, Z/E forms), 7.13 (1H, broad), 7.20 (1H, broad), 7.68 (1H, broad), 7.78 (1H, broad), 8.45 (1H, d, J=5.0 Hz). MW 434.43. LCMS t_{R} (min): 1.75. MS (APCI), m/z 435.08 [M+H]⁺. HPLC t_{R} (min): 11.58.

### 18. [4-(4-Benzothiazol-2-yl-piperazin-1-yl)-6-(2,2,2-trifluoro-ethoxy)-[1,3,5]triazin-2-yl]-(5-methyl-furan-2-ylmethyl)-amine

Yield 295 mg, 63%. ¹H-NMR (400 MHz, DMSO-D₆) δ_{H}: 2.20 (3H, s), 3.65 (4H, m), 3.92 (4H, broad), 4.40 (2H, broad d, J=7.5 Hz), 4.93 (2H, superposition of two q, J=7.5 Hz, Z/E forms), 5.98 (1H, broad), 6.07-6.20 (1H, broad, Z/E forms), 7.08 (1H, t, J=8.5 Hz), 7.28 (1H, t, J=8.5 Hz), 7.47 (1H, d, J=8.5 Hz), 7.76 (1H, d, J=8.5 Hz), 7.87 (1H, broad). MW 505.53. LCMS t_{R} (min): 2.12. MS (APCI), m/z 506.07 [M+H]⁺. HPLC t_{R} (min): 15.88. M_{P} 162-164° C.

### 19. [4-[4-(4-Methyl-benzyl)-piperazin-1-yl]-6-(2,2,2-trifluoro-ethoxy)-[1,3,5]triazin-2-yl]-(5-methyl-furan-2-ylmethyl)-amine

Yield 110 mg, 25%. ¹H-NMR (400 MHz, DMSO-D₆) δ_{H}: 2.20 (3H, s), 2.29 (3H, s), 2.37 (4H, broad), 3.45 (2H, s), 3.72 (4H, broad), 4.35 (2H, broad), 4.89 (2H, superposition of two q, J=7.5 Hz, Z/E forms), 5.94 (1H, broad), 6.09 (1H, broad), 7.12 (2H, d, J=8.5 Hz), 7.18 (2H, d, J=8.5 Hz), 7.74 (1H, broad). MW 476.51. LCMS t_{R} (min): 1.71. MS (APCI) m/z 477.24 [M+H]⁺. HPLC t_{R} (min) 12.32. M_{P} 121-123° C.

### 20. [4-(4-Ethanesulfonyl-piperazin-1-yl)-6-(2,2,2-trifluoro-ethoxy)-[1,3,5]triazin-2-yl]-(5-methyl-furan-2-ylmethyl)-amine

Yield 127 mg, 30%. ¹H-NMR (400 MHz, DMSO-D₆) δ_{H}: 1.22 (3H, broad t, J=7.5 Hz), 2.20 (3H, s), 3.05 (2H, broad q, J=7.5 Hz), 3.20 (4H, broad), 3.80 (4H, broad), 4.39 (2H, d, J=7.5 Hz), 4.91 (2H, superposition of two q, J=7.5 Hz, Z/E forms), 5.95 (1H, broad), 6.10 (1H, broad), 7.88 (1H, broad). MW 464.47. LCMS t_{R} (min): 1.87. MS (APCI) m/z 465.04 [M+H]⁺. HPLC t_{R} (min) 14.58. M_{P} 190-192° C.

### 21. [4-[4-(2-Methoxy-5-methyl-benzenesulfonyl)-piperazin-1-yl]-6-(2,2,2-trifluoro-ethoxy)-[1,3,5]triazin-2-yl]-(5-methyl-furan-2-ylmethyl)-amine

Yield 160 mg, 31%. ¹H-NMR (400 MHz, DMSO-D₆) δ_{H}: 2.19 (3H, s), 2.31 (3H, s), 3.12 (4H, m), 3.72 (4H, broad), 3.82 (3H, s), 4.37 (2H, broad), 4.90 (2H, superposition of two q, J=7.5 Hz, Z/E forms), 5.92 (1H, broad), 6.10 (1H, broad), 7.13 (1H, d, J=8.5 Hz), 7.43 (1H, broad doublet, J=8.5 Hz), 7.55 (1H, broad), 7.84 (1H, broad). MW 556.57. LCMS t_{R} (min): 2.10. MS (APCI) m/z 557.36 [M+H]⁺. HPLC t_{R} (min) 16.09. M_{P} 170-172° C.

### 22. (5-Methyl-furan-2-ylmethyl)-[4-[4-(toluene-4-sulfonyl)-piperazin-1-yl]-6-(2,2,2-trifluoro-ethoxy)-[1,3,5]triazin-2-yl]-amine

Yield 146 mg, 30%. ¹H-NMR (400 MHz, DMSO-D₆) δ_{H}: 2.20 (3H, s), 2.40 (3H, s), 2.90 (4H, m), 3.80 (4H, broad, Z/E forms), 4.34 (2H, broad, Z/E forms), 4.87 (2H, superposition of two broad q, J=7.5 Hz, Z/E forms), 5.93 (1H, d, J=3.6 Hz), 6.07 (1H, broad), 7.43 (2H, d, J=8.5 Hz), 7.61 (2H, d, J=8.5 Hz), 7.83 (1H, broad). MW 526.54. LCMS t_{R} (min): 2.07. MS (APCI), m/z 527.05 [M+H]⁺. HPLC t_{R} (min): 16.45. M_{P} 172-174° C.

### 23. [4-(4-Methyl-[1,4]bipiperidinyl-1′-yl)-6-(2,2,2-trifluoro-ethoxy)-[1,3,5]triazin-2-yl]-(5-methyl-furan-2-ylmethyl)-amine

Yield 182 mg, 50%. ¹H-NMR (400 MHz, DMSO-D₆) δ_{H}: 0.88 (3H, d, J=7.5 Hz), 1.09 (2H, m), 1.30 (3H, m), 1.57 (2H, m), 1.77 (2H, m), 2.10 (2H, m), 2.20 (3H, s), 2.80 (4H, broad m, Z/E forms), 3.25 (1H, m), 4.38 (2H, broad d, J=7.5 Hz, Z/E forms), 4.50-4.75 (2H, broad, Z/E forms), 4.89 (2H, superposition of two broad q, J=7.5 Hz, Z/E forms), 5.95 (1H, d, J=3.6 Hz), 6.10 (1H, d, J=3.6 Hz), 7.72 (1H, broad t, J=7.5 Hz, Z/E forms). MW 468.53. LCMS t_{R} (min): 1.56. MS (APCI) m/z 469.19 [M+H]⁺. HPLC t_{R} (min) 11.64. M_{P} 159-161° C.

### 24. (5-Methyl-furan-2-ylmethyl)-[4-(4-morpholin-4-yl-piperidin-1-yl)-6-(2,2,2-trifluoro-ethoxy)-[1,3,5]triazin-2-yl]-amine

Yield 110 mg, 31%. ¹H-NMR (400 MHz, DMSO-D₆) δ_{H}: 1.28 (2H, broad, Z/E forms), 1.80 (2H, broad, Z/E forms), 2.20 (3H, s), 2.41 (4H, m), 2.88 (2H, m), 3.20 (1H, m), 3.55 (4H, broad, Z/E forms), 4.37 (2H, broad doublet, J=7.5 Hz, Z/E forms), 4.58 (2H, broad, Z/E forms), 4.89 (2H, broad, Z/E forms), 5.95 (1H, d, J=3.6 Hz), 6.09 (1H, broad, Z/E forms), 7.73 (1H, broad triplet, J=7.5 Hz, Z/E forms). MW 456.47. LCMS t_{R} (min): 1.49. MS (APCI) m/z 547.13 [M+H]⁺. HPLC t_{R} (min) 10.51. M_{P} 143-145° C.

### 25. [4-[4-(4-Fluoro-benzyl)-piperazin-1-yl]-6-(2,2,2-trifluoro-ethoxy)-[1,3,5]triazin-2-yl]-(5-methyl-furan-2-ylmethyl)-amine

Yield 90 mg, 20%. ¹H-NMR (400 MHz, DMSO-D₆) δ_{H}: 2.20 (3H, s), 2.36 (4H, m), 3.48 (2H, s), 3.70 (4H, broad, Z/E forms), 4.36 (2H, broad, Z/E forms), 4.88 (2H, superposition of two q, J=7.5 Hz, Z/E forms), 5.92 (1H, d, J=3.6 Hz), 6.07 (1H, broad, Z/E forms), 7.12 (2H, dd, J=8.0 Hz), 7.33 (2H, broad, Z/E forms), 7.74 (1H, broad, Z/E forms). MW 480.47. LCMS t_{R} (min): 1.71. MS (APCI) m/z 481.10 [M+H]⁺. HPLC t_{R} (min) 11.99. M_{P} 144-146° C.

### 26. 1-{1-[4-[(5-Methyl-furan-2-ylmethyl)-amino]-6-(2,2,2-trifluoro-ethoxy)-[1,3,5]triazin-2-yl]-piperidin-4-yl}-1,3-dihydro-benzoimidazol-2-one

Yield 218 mg, 56%. ¹H-NMR (400 MHz, DMSO-D₆) δ_{H}: 1.78 (2H, m), 2.23 (5H, m), 3.00 (2H, broad), 4.42 (2H, broad), 4.48 (1H, broad), 4.80 (2H, broad), 4.92 (2H, broad), 5.95 (1H, broad), 6.10 (1H, broad), 6.97 (3H, broad), 7.20 (1H, broad), 7.80 (1H, broad), 10.75 (1H, broad). MW 503.49. LCMS t_{R} (min): 1.90. MS (APCI) m/z 504.12 [M+H]⁺. HPLC t_{R} (min) 4.36. M_{P} 191-193° C.

### 27. N-Methyl-N′-(5-methyl-furan-2-ylmethyl)-N-(2-pyridin-2-yl-ethyl)-6-(2,2,2-trifluoro-ethoxy)-[1,3,5]triazine-2,4-diamine

Yield 80 mg, 10%. ¹H-NMR (400 MHz, DMSO-D₆) δ_{H}: 2.12-2.28 (3H, broad, Z/E forms), 3.00 (5H, m), 3.88 (2H, broad), 4.39 (2H, d, J=7.5 Hz), 4.88 (2H, broad), 5.93 (1H, broad), 6.09 (1H, broad), 7.19 (2H, broad), 7.67 (2H, broad), 8.47 (1H, broad). MW 422.41. LCMS t_{R} (min): 1.61. MS (APCI), m/z 423.09 [M+H]⁺. HPLC t_{R} (min): 10.80. M_{P} 43-45° C.

### 28. N,N-Diisobutyl-N′-(5-methyl-furan-2-ylmethyl)-6-(2,2,2-trifluoro-ethoxy)-[1,3,5]triazine-2,4-diamine

Yield 170 mg, 66%. ¹H-NMR (400 MHz, DMSO-D₆) δ_{H}: 0.80 (12H, broad d, J=7.5 Hz), 2.04 (1H, broad), 2.20 (3H, s), 3.34 (2H, broad), 4.35 (2H, broad d, J=7.5 Hz), 4.88 (2H, superposition of two quartets, J=7.5 Hz, Z/E forms), 5.94 (1H, broad), 6.00-6.10 (1H, broad, Z/E forms), 7.65 (1H, broad). MW 415.46. LCMS t_{R} (min): 2.39. MS (APCI), m/z 416.09 [M+H]⁺. HPLC t_{R} (min): 18.76. M_{P} 40-42° C.

### 29. (5-Methyl-furan-2-ylmethyl)-[4-(4-pyridin-3-ylmethyl-piperazin-1-yl)-6-(2,2,2-trifluoro-ethoxy)-[1,3,5]triazin-2-yl]-amine

Yield 75 mg, 17%. ¹H-NMR (400 MHz, DMSO-D₆) δ_{H}: 2.21 (3H, s), 2.42 (4H, m), 3.55 (2H, s), 3.75 (4H, broad), 4.38 (2H, broad), 4.91 (2H, superposition of two q, J=7.5 Hz, Z/E forms), 5.95 (1H, broad), 6.12 (1H, broad), 7.35 (1H, broad t, J=8.0/5.0 Hz), 7.72 (1H, d, J=8.5 Hz), 7.77 (1H, broad), 8.48 (1H, broad d, J=5.0 Hz), 8.52 (1H, s). MW 463.47. LCMS t_{R} (min): 1.47. MS (APCI) m/z 464.03 [M+H]⁺. HPLC t_{R} (min) 9.80. M_{P} 117-119° C.

### 30. (5-Methyl-furan-2-ylmethyl)-[4-[4-(piperidine-1-sulfonyl)-piperazin-1-yl]-6-(2,2,2-trifluoro-ethoxy)-[1,3,5]triazin-2-yl]-amine

Yield 411 mg, 30%. ¹H-NMR (400 MHz, DMSO-D₆) δ_{H}: 1.50 (6H, m), 2.20 (3H, s), 3.15 (8H, m), 3.79 (4H, broad, Z/E forms), 4.38 (2H, d, J=7.5 Hz, Z/E forms), 4.90 (2H, broad q, J=7.5 Hz, Z/E forms), 5.95 (1H, d, J=3.6 Hz), 6.07-6.15 (1H, two broad signals, Z/E forms), 7.85 (1H, broad t, J=7.5 Hz, Z/E forms). MW 519.55. LCMS t_{R} (min): 2.05. MS (APCI) m/z 520.09 [M+H]⁺. HPLC t_{R} (min) 16.34. M_{P} 181-184° C.

### 31. 3-[4-[(5-Methyl-furan-2-ylmethyl)-amino]-6-(2,2,2-trifluoro-ethoxy)-[1,3,5]triazin-2-yl]-1,2,3,4,5,6-hexahydro-1,5-methano-pyrido[1,2-a][1,5]dia° C.in-8-one

Yield 262 mg, 73%. ¹H-NMR (400 MHz, DMSO-D₅) δ_{H}: 1.98 (2H, m), 2.20 (3H, broad), 3.09 (4H, m), 3.65 (1H, broad), 3.80-3.92 (1H, broad, Z/E forms), 4.12-4.27 (1H, broad, Z/E forms), 4.32 (1H, broad), 4.60 (1H, broad), 4.75 (2H, broad), 4.82 (1H, broad), 5.94 (1H, broad), 5.98-6.05 (1H, broad, Z/E forms), 6.10 (1H, broad), 6.18 (1H, broad), 7.26 (1H, broad t, J=8.5 Hz), 7.70 (1H, broad). MW 476.46. LCMS t_{R} (min): 1.71. MS (APCI), m/z 477.00 [M+H]⁺. HPLC t_{R} (min): 15.73. M_{P} 152-154° C.

### 32. {4-[4-[(5-Methyl-furan-2-ylmethyl)-amino]-6-(2,2,2-trifluoro-ethoxy)-[1,3,5]triazin-2-yl]-piperazin-1-yl}-morpholin-4-yl-methanone

Yield 80 mg, 18%. ¹H-NMR (400 MHz, DMSO-D₆) δ_{H}: 2.22 (3H, s), 3.20 (8H, m), 3.58 (4H, broad, Z/E forms), 3.70 (4H, broad, Z/E forms), 4.38 (2H, broad d, J=7.5 Hz, Z/E forms), 4.90 (2H, broad q, J=7.5 Hz, Z/E forms), 5.95 (1H, d, J=3.6 Hz), 6.10 (1H, broad, Z/E forms), 7.80 (1H, broad t, Z/E forms). MW 485.47. LCMS t_{R} (min): 1.76. MS (APCI) m/z 486.25 [M+H]⁺. HPLC t_{R} (min) 13.28. M_{P} 150-153° C.

### 33. (5-Methyl-furan-2-ylmethyl)-[4-(4-pyrrolidin-1-yl-piperidin-1-yl)-6-(2,2,2-trifluoro-ethoxy)-[1,3,5]triazin-2-yl]-amine

Yield 202 mg, 60%. ¹H-NMR (400 MHz, DMSO-D₆) δ_{H}: 1.28 (2H, m), 1.65 (4H, m), 1.82 (2H, m), 2.20 (3H, s), 2.23 (1H, m), 2.45 (4H, broad), 3.03 (2H, m), 4.34 (2H, broad d, J=7.5 Hz), 4.42 (2H, broad, Z/E forms), 4.89 (2H, superposition of two broad q, J=7.5 Hz), 5.94 (1H, d, J=3.6 Hz), 6.10 (1H, d, J=3.6 Hz), 7.72 (1H, broad t, J=7.5 Hz). MW 440.47. LCMS t_{R} (min): 1.54. MS (APCI), m/z 441.17 [M+H]⁺. HPLC t_{R} (min): 10.38. M_{P} 121-123° C.

### 34. (5-Methyl-furan-2-ylmethyl)-[4-[4-(4-methyl-quinolin-2-yl)-piperazin-1-yl]-6-(2,2,2-trifluoro-ethoxy)-[1,3,5]triazin-2-yl]-amine

Yield 82 mg, 26%. ¹H-NMR (400 MHz, DMSO-D₆) δ_{H}: 2.21 (3H, s), 2.59 (3H, s), 3.75 (4H, broad), 3.85 (4H, broad), 4.42 (2H, broad), 4.95 (2H, superposition of two q, J=7.5 Hz, Z/E forms), 5.95 (1H, broad), 6.10-6.13 (1H, two broad signals, Z/E forms), 7.12 (1H, broad), 7.25 (1H, t, J=8.5 Hz), 7.52 (1H, t, J=8.5 Hz), 7.58 (1H, d, J=8.5 Hz), 7.81 (1H, broad), 7.87 (1H, broad). MW 513.53. LCMS t_{R} (min): 1.72. MS (APCI) m/z 514.19 [M+H]⁺. HPLC t_{R} (min) 12.71. M_{P} 82-84° C.

### 35. 4-[4-[(5-Methyl-furan-2-ylmethyl)-amino]-6-(2,2,2-trifluoro-ethoxy)-[1,3,5]triazin-2-yl]-piperazine-1-carboxylic acid diethylamide

Yield 218 mg, 50%. ¹H-NMR (400 MHz, DMSO-D₆) δ_{H}: 1.08 (6H, broad, Z/E forms), 2.21 (3H, s), 3.13 (8H, broad m, Z/E forms), 3.75 (4H, broad, Z/E forms), 4.40 (2H, broad d, J=7.5 Hz, Z/E forms), 4.91 (2H, superposition of two q, J=7.5 Hz, Z/E forms), 5.95 (1H, d, J=3.6 Hz), 6.10 (1H, broad, Z/E forms), 7.80 (1H, broad, Z/E forms). MW 471.49. LCMS t_{R} (min): 1.98. MS (APCI) m/z 472.07 [M+H]⁺. HPLC t_{R} (min) 15.17. M_{P} 116-118° C.

### 36. [4-(4-Benzyloxy-piperidin-1-yl)-6-(2,2,2-trifluoro-ethoxy)-[1,3,5]triazin-2-yl]-(5-methyl-furan-2-ylmethyl)-amine

Yield 75 mg, 20%. ¹H-NMR (400 MHz, DMSO-D₆) δ_{H}: 1.45 (2H, broad, Z/E forms), 1.86 (2H, broad, Z/E forms), 2.20 (3H, s), 3.40 (2H, broad, Z/E forms), 3.68 (1H, broad, Z/E forms), 4.10 (2H, broad, Z/E forms), 4.38 (2H, d, J=7.5 Hz), 4.55 (2H, s), 4.90 (2H, superposition of two q, J=7.5 Hz, Z/E forms), 5.95 (1H, broad, Z/E forms), 6.08 (1H, broad, Z/E forms), 7.25 (1H, broad, Z/E forms), 7.32 (4H, m), 7.73 (1H, broad t, J=7.5 Hz, Z/E forms). MW 477.49. LCMS t_{R} (min): 2.19. MS (APCI) m/z 478.11 [M+H]⁺. HPLC t_{R} (min) 17.44. M_{P} 111-113° C.

### 37. [4-[4-(4-Fluoro-phenyl)-piperazin-1-yl]-6-(2,2,2-trifluoro-ethoxy)-[1,3,5]triazin-2-yl]-(5-methyl-furan-2-ylmethyl)-amine

Yield 280 mg, 65%. ¹H-NMR (400 MHz, DMSO-D₆) δ_{H}: 2.20 (3H, s), 3.09 (4H, m), 3.86 (4H, broad), 4.40 (2H, d, J=7.5 Hz), 4.90 (2H, superposition of two q, J=7.5 Hz, Z/E forms), 5.95 (1H, broad), 6.10 (1H, broad), 6.98 (2H, broad), 7.04 (2H, t, J=8.5 Hz), 7.80 (1H, broad t, J=7.5 Hz). MW 466.44. LCMS t_{R} (min): 2.23. MS (APCI) m/z 467.09 [M+H]⁺. HPLC t_{R} (min) 16.52. M_{P} 119-121° C.

### 38. [4-(4-Benzyloxy-piperidin-1-yl)-6-(2,2,2-trifluoro-ethoxy)-[1,3,5]triazin-2-yl]-(5-methyl-furan-2-ylmethyl)-amine

Yield 207 mg, 47%. ¹H-NMR (400 MHz, DMSO-D₆) δ_{H}: 1.40 (2H, broad, Z/E forms), 1.70 (1H, broad, Z/E forms), 1.80 (1H, broad, Z/E forms), 1.92 (1H, broad, Z/E forms), 2.20 (3H, s), 2.93-3.08 (2H, two broad signals, Z/E forms), 4.15 (2H, superposition of two q, J=7.5 Hz, Z/E forms), 4.35 (2H, broad, Z/E forms), 4.45-4.70 (2H, two broad signals, Z/E forms), 4.85 (2H, broad, Z/E forms), 5.85-5.98 (1H, two broad signals, Z/E forms), 6.09 (1H, broad, Z/E forms), 6.80 (1H, broad, Z/E forms), 6.95 (1H, broad, Z/E forms), 7.70 (2H, broad, Z/E forms), 8.13 (1H, d, J=5.0 Hz, Z/E forms). MW 478.48. LCMS t_{R} (min): 2.14. MS (APCI) m/z 479.04 [M+H]⁺. HPLC t_{R} (min) 16.44. M_{P} 107-109° C.

### 39. (5-Methyl-furan-2-ylmethyl)-[4-[4-(2-pyridin-2-yl-ethyl)-piperazin-1-yl]-6-(2,2,2-trifluoro-ethoxy)-[1,3,5]triazin-2-yl]-amine

Yield 280 mg, 63%. ¹H-NMR (400 MHz, DMSO-D₆) δ_{H}: 2.20 (3H, s), 2.45 (4H, m), 2.70 (2H, broad, Z/E forms), 2.90 (2H, broad t, J=7.5 Hz, Z/E forms), 3.70 (4H, broad, Z/E forms), 4.38 (2H, broad, Z/E forms), 4.90 (2H, broad, Z/E forms), 5.95 (1H, broad, Z/E forms), 6.10 (1H, broad, Z/E forms), 7.17 (1H, broad dd, J=8.0/5.0 Hz, Z/E forms), 7.28 (1H, d, J=8.0 Hz), 7.67 (1H, t, J=8.0 Hz), 7.75 (1H, broad t, J=7.5 Hz, Z/E forms), 8.45 (1H, d, J=5.0 Hz). MW 477.49. LCMS t_{R} (min): 1.55. MS (APCI) m/z 478.13 [M+H]⁺. HPLC t_{R} (min) 9.98. M_{P} 116-119° C.

### 40. (5-Methyl-furan-2-ylmethyl)-[4-[4-(4-methyl-furazan-3-ylmethyl)-piperazin-1-yl]-6-(2,2,2-trifluoro-ethoxy)-[1,3,5]triazin-2-yl]-amine

Yield 285 mg, 66%. ¹H-NMR (400 MHz, DMSO-D₆) δ_{H}: 2.20 (3H, s), 2.38 (4H, m), 2.45 (3H, s), 3.68 (4H, m), 3.74 (2H, s), 4.35 (2H, broad d, J=7.5 Hz), 4.88 (2H, superposition of two q, J=7.5 Hz, Z/E forms), 5.93 (1H, d, J=3.6 Hz), 6.07 (1H, d, J=3.6 Hz), 7.78 (1H, broad t, J=7.5 Hz). MW 468.44. LCMS t_{R} (min): 1.97. MS (APCI) m/z 469.07 [M+H]⁺. HPLC t_{R} (min) 12.37. M_{P} 148-150° C.

### 41. N,N-Dimethyl-2-{4-[4-[(5-methyl-furan-2-ylmethyl)-amino]-6-(2,2,2-trifluoro-ethoxy)-[1,3,5]triazin-2-yl]-piperazin-1-yl}-acetamide

Yield 287 mg, 68%. ¹H-NMR (400 MHz, DMSO-D₆) δ_{H}: 2.20 (3H, s), 2.48 (4H, m), 2.81 (3H, s), 3.02 (3H, s), 3.18 (2H, s), 3.72 (4H, broad, Z/E forms), 4.38 (2H, broad d, J=7.5 Hz, Z/E forms), 4.89 (2H, broad q, J=7.5 Hz, Z/E forms), 5.95 (1H, broad, Z/E forms), 6.10 (1H, broad, Z/E forms), 7.78 (1H, broad t, Z/E forms). MW 457.46. LCMS t_{R} (min): 1.52. MS (APCI) m/z 458.09 [M+H]⁺. HPLC t_{R} (min) 10.46. M_{P} 114-116° C.

### 42. (5-Methyl-furan-2-ylmethyl)-[4-(4-thiazol-2-yl-piperazin-1-yl)-6-(2,2,2-trifluoro-ethoxy)-[1,3,5]triazin-2-yl]-amine

Yield 278 mg, 66%. ¹H-NMR (400 MHz, DMSO-D₆) δ_{H}: 2.20 (3H, s), 3.45 (4H, m), 3.88 (4H, broad, Z/E forms), 4.40 (2H, broad d, J=7.5 Hz), 4.92 (2H, superposition of two q, J=7.5 Hz, Z/E forms), 5.95 (1H, broad, Z/E forms), 6.07-6.16 (1H, two broad signals, Z/E forms), 6.85 (1H, broad, Z/E forms), 7.20 (1H, broad, Z/E forms), 7.87 (1H, broad t, J=7.5 Hz, Z/E forms). MW 455.47. LCMS t_{R} (min): 1.89. MS (APCI) m/z 456.04 [M+H]⁺. HPLC t_{R} (min) 12.12. M_{P} 135-137° C.

### 43. [4-[4-(4-Isobutyl-benzenesulfonyl)-piperazin-1-yl]-6-(2,2,2-trifluoro-ethoxy)-[1,3,5]triazin-2-yl]-(5-methyl-furan-2-ylmethyl)-amine

Yield 237 mg, 45%. ¹H-NMR (400 MHz, DMSO-D₆) δ_{H}: 0.85 (6H, d, J=7.5 Hz), 1.88 (1H, m), 2.20 (3H, s), 2.55 (2H, d, J=7.5 Hz), 2.92 (4H, broad, Z/E forms), 3.80 (4H, broad, Z/E forms), 4.35 (2H, broad, Z/E forms), 4.88 (2H, superposition of two q, J=7.5 Hz, Z/E forms), 5.95 (1H, d, J=3.6 Hz), 6.08 (1H, broad, Z/E forms), 7.41 (2H, d, J=8.5 Hz), 7.64 (2H, d, J=8.5 Hz), 7.83 (1H, broad, Z/E forms). MW 568.62. LCMS t_{R} (min): 2.31. MS (APCI) m/z 569.10 [M+H]⁺. HPLC t_{R} (min) 17.99. M_{P} 177-179° C.

### 44. [4-[4-(7-Chloro-quinolin-4-yl)-piperazin-1-yl]-6-(2,2,2-trifluoro-ethoxy)-[1,3,5]triazin-2-yl]-(5-methyl-furan-2-ylmethyl)-amine

Yield 376 mg, 76%. ¹H-NMR (400 MHz, DMSO-D₆) δ_{H}: 2.21 (3H, s), 3.20 (4H, broad), 4.01 (4H, broad), 4.41 (2H, broad d, J=7.5 Hz), 4.95 (2H, superposition of two q, J=7.5 Hz, Z/E forms), 5.98 (1H, d, J=3.6 Hz), 6.12 (1H, broad), 7.03 (1H, broad d, J=5.0 Hz), 7.56 (1H, d, J=8.5 Hz), 7.85 (1H, broad t, J=7.5 Hz), 7.89 (1H, s), 8.13 (1H, d, J=8.5 Hz), 8.72 (1H, broad d, J=5.0 Hz). MW 533.94. LCMS t_{R} (min): 1.74. MS (APCI) m/z 534.17, 536.16 [M+H]⁺. HPLC t_{R} (min) 12.54. M_{P} 249-252° C.

### 45. (5-Methyl-furan-2-ylmethyl)-{4-(2,2,2-trifluoro-ethoxy)-6-[4-(1,3,5-trimethyl-1H-pyrazol-4-ylmethyl)-piperazin-1-yl]-[1,3,5]triazin-2-yl}-amine

Yield 287 mg, 63%. ¹H-NMR (400 MHz, DMSO-D₆) δ_{H}: 2.02 (3H, s), 2.12 (3H, s), 2.20 (3H, s), 2.29 (4H, broad), 3.20 (3H, s), 3.60 (2H, s), 3.69 (4H, m), 4.35 (2H, broad), 4.87 (2H, superposition of two q, J=7.5 Hz, Z/E forms), 5.93 (1H, d, J=3.6 Hz), 6.07 (1H, d, J=3.6 Hz), 7.75 (1H, broad). MW 494.52. LCMS t_{R} (min): 1.59. MS (APCI) m/z 494.92 [M+H]⁺. HPLC t_{R} (min) 10.67. M_{P} 115-118° C.

### 46. [4-[4-(4-Fluoro-benzenesulfonyl)-piperazin-1-yl]-6-(2,2,2-trifluoro-ethoxy)-[1,3,5]triazin-2-yl]-(5-methyl-furan-2-ylmethyl)-amine

Yield 370 mg, 75%. ¹H-NMR (400 MHz, DMSO-D₆) δ_{H}: 2.20 (3H, s), 2.94 (4H, broad), 3.80 (4H, broad), 4.34 (2H, broad), 4.88 (2H, superposition of two q, J=7.5 Hz, Z/E forms), 5.92 (1H, broad), 6.09 (1H, broad), 7.45 (2H, broad t, J=8.5 Hz), 7.81 (3H, broad). MW 530.50. LCMS t_{R} (min): 2.10. MS (APCI) m/z 531.02 [M+H]⁺. HPLC t_{R} (min) 16.12. M_{P} 189-191° C.

### 47. [4-[4-(5-Methyl-1H-benzoimidazol-2-yl)-piperidin-1-yl]-6-(2,2,2-trifluoro-ethoxy)-[1,3,5]-triazin-2-yl]-(5-methyl-furan-2-ylmethyl)-amine

Yield 60 mg, 13%. ¹H-NMR (400 MHz, DMSO-D₆) δ_{H}: 1.71 (2H, m), 2.05 (2H, broad), 2.21 (3H, s), 2.41 (3H, s), 3.15 (3H, broad), 4.38 (2H, broad), 4.60-4.68 (2H, broad, Z/E forms), 4.98 (2H, superposition of two q, J=7.5 Hz, Z/E forms), 5.95 (1H, d, J=3.6 Hz), 6.15 (1H, broad), 6.91 (1H, d, J=8.5 Hz), 7.22 (1H, broad), 7.32 (1H, broad), 7.78 (1H, broad), 11.95 (1H, broad). MW 501.52. LCMS t_{R} (min): 1.61. MS (APCI) m/z 502.15 [M+H]⁺. HPLC t_{R} (min) 12.18. M_{P} 101-103° C.

### 48. (5-Methyl-furan-2-ylmethyl)-[4-[4-(2-methyl-imidazol-1-ylmethyl)-piperidin-1-yl]-6-(2,2,2-trifluoro-ethoxy)-[1,3,5]triazin-2-yl]-amine

Yield 184 mg, 64%. ¹H-NMR (400 MHz, DMSO-D₆) δ_{H}: 1.22 (2H, m), 1.68 (2H, m), 1.94 (1H, m), 2.28 (3H, s), 2.39 (3H, s), 2.80 (2H, m), 3.72 (2H, d, J=7.5 Hz), 4.51 (2H, d, J=7.5 Hz), 4.70 (2H, broad), 4.80 (2H, broad), 5.30 (1H, broad), 5.88 (1H, d, J=3.6 Hz), 6.09 (1H, d, J=3.6 Hz), 6.78 (1H, s), 6.92 (1H, s). MW 465.48. LCMS t_{R} (min): 1.58. MS (APCI) m/z 466.13 [M+H]⁺. HPLC t_{R} (min) 11.16. M_{P} 92-94° C.

### 49. 2-{1-[4-[(5-Methyl-furan-2-ylmethyl)-amino]-6-(2,2,2-trifluoro-ethoxy)-[1,3,5]triazin-2-yl]-piperidin-4-yl}-4-thiophen-2-yl-2H-[1,2,3]oxadiazol-5-one

Yield 253 mg, 76%. ¹H-NMR (400 MHz, DMSO-D₆) δ_{H}: 1.72 (2H, m), 1.96 (2H, m), 2.20 (3H, s), 3.06 (2H, broad), 4.25 (1H, m), 4.38 (2H, broad), 4.68 (2H, broad d, J=7.5 Hz), 4.90 (2H, superposition of two q, J=7.5 Hz, Z/E forms), 5.94 (1H, d, J=3.6 Hz), 6.10 (1H, d, J=3.6 Hz), 7.21 (1H, d/d, J=5.4/4.0 Hz), 7.63 (1H, d, J=4.0 Hz), 7.78 (1H, broad), 7.84 (1H, d, J=5.4 Hz). MW 537.52. LCMS t_{R} (min): 2.18. MS (APCI) m/z 538.04 [M+H]⁺. HPLC t_{R} (min) 16.32. M_{P} 196-198° C.

### 50. 1-[4-[(5-Methyl-furan-2-ylmethyl)-amino]-6-(2,2,2-trifluoro-ethoxy)-[1,3,5]triazin-2-yl]-piperidine-3-carbonitrile

Yield 90 mg, 29%. ¹H-NMR (400 MHz, DMSO-D₆) δ_{H}: 1.60 (2H, broad m), 1.90 (2H, broad m), 2.20 (3H, s), 3.05 (1H, broad), 3.50-3.97 (2H, broad, Z/E forms), 3.70-4.20 (2H, broad, Z/E forms), 4.40 (2H, broad d, J=8.5 Hz), 4.91 (2H, superposition of two q, J=7.5 Hz, Z/E forms), 5.95 (1H, d, J=3.6 Hz), 6.10 (1H, broad), 7.88 (1H, broad). MW 396.38. LCMS t_{R} (min): 1.91. MS (APCI) m/z 397.04 [M+H]⁺. HPLC t_{R} (min) 14.88. M_{P} 120-122° C.

### 51. [4-(1,1-Dioxo-1lambda*6*-thiomorpholin-4-yl)-6-(2,2,2-trifluoro-ethoxy)-[1,3,5]triazin-2-yl]-(5-methyl-furan-2-ylmethyl)-amine

Yield 140 mg, 43%. ¹H-NMR (400 MHz, DMSO-D₆) δ_{H}: 2.21 (3H, s), 3.12 (4H, broad), 4.16 (4H, broad), 4.40 (2H, broad), 4.92 (2H, superposition of two q, J=7.5 Hz, Z/E forms), 5.95 (1H, broad), 6.11 (1H, broad), 7.99 (1H, broad). MW 421.40. LCMS t_{R} (min): 1.81. MS (APCI) m/z 421.95 [M+H]⁺. HPLC t_{R} (min) 13.50. M_{P} 197-199° C.

### 52. 6-Chloro-N-(4-fluoro-3-trifluoromethyl-phenyl)-N′-(5-methyl-furan-2-ylmethyl)-[1,3,5]triazine-2,4-diamine

¹H-NMR (400 MHz, DMSO-D₆) δ_{H}: 2.15-2.25 (3H, two s, Z/E forms), 4.42 (2H, broad d, J=7.5 Hz), 5.93-5.97 (1H, two broad signals, Z/E forms), 6.08-6.13 (1H, two broad signals, Z/E forms), 7.45 (1H, superposition of two m, J=8.5 Hz, Z/E forms), 7.90-8.05 (1H, two broad signals, Z/E forms), 8.05-8.29 (1H, two broad signals, Z/E forms), 8.49-8.59 (1H, two broad signals, Z/E forms), 10.18-10.32 (1H, two broad signals, Z/E forms). MW 401.75. LCMS t_{R} (min): 2.05. MS (APCI), m/z 401.91, 403.88 [M+H]⁺. HPLC t_{R} (min): 16.19. M_{P} 180-182° C.

### 53. [4-(4-Fluoro-phenyl)-6-(2,2,2-trifluoro-ethoxy)-[1,3,5]triazin-2-yl]-(5-methyl-furan-2-ylmethyl)-amine

Yield 277 mg, 47%. ¹H-NMR (400 MHz, DMSO-D₆) δ_{H}: 2.21 (3H, s), 4.50-4.61 (2H, two d, J=7.5 Hz, Z/E forms), 5.10 (2H, superposition of two q, J=7.5 Hz, Z/E forms), 5.99 (1H, broad peak, Z/E forms), 6.20 (1H, d, J=3.6 Hz), 7.36 (2H, broad t, J=8.5/8.0 Hz), 8.38-8.41 (2H, two broad m, Z/E forms), 8.54-8.66 (1H, two broad t, J=7.5 Hz, Z/E forms). MW 382.32. LCMS t_{R} (min): 2.11. MS (APCI+), m/z 382.85 [M+H]⁺. HPLC t_{R} (min): 17.58. M_{P} 155-157° C.

### 54. (5-Methyl-furan-2-ylmethyl)-[4-(2,2,2-trifluoro-ethoxy)-6-(3-trifluoromethyl-phenoxy)-[1,3,5]triazin-2-yl]-amine

To a solution of compound 13 (300 mg, 0.93 mmol) in acetonitrile (5 mL) m-trifluoromethyl-phenol (301 mg, 1.86 mmol) and K₂CO₃ (385 mg, 2.79 mmol) were added. The mixture was stirred at 50° C. for 1 hour and diluted with water. The formed solid was collected by filtration, washed with water and hexane and dried giving the (350 mg, 84%). ¹H-NMR (400 MHz, DMSO-D₆) δ_{H}: 2.20 (3H, s), 4.24-4.41 (2H, two d, J=7.5 Hz, Z/E forms), 4.85-5.05 (2H, two q, J=7.5 Hz, Z/E forms), 5.82-5.91 (1H, two d, J=3.6 Hz, Z/E forms), 5.95-6.15 (1H, two d, J=3.6 Hz, Z/E forms), 7.55 (1H, t, J=8.5 Hz), 7.63 (1H, s), 7.65 (2H, broad, Z/E forms), 8.65 (1H, broad t, J=8.5 Hz). MW 448.33. LCMS t_{R} (min): 2.10. MS (APCI) m/z 449.11 [M+H]⁺. HPLC t_{R} (min) 17.01. M_{P} 116-118° C.

### Preparation of O,C(Ar),N-Triazines in Table 13

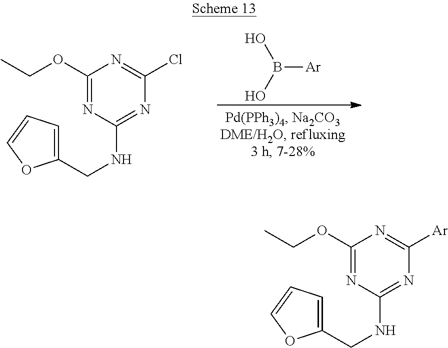

General procedure: A mixture of chloro-triazine (255 mg, 1.0 mmol), boronic acid (1.0 mmol), Pd(PPh₃)₄ (120 mg, 0.1 mol, 10 mol %), Na₂CO₃ (424 mg, 4.0 mmol), dimethoxy ethane (3 mL) and water (3 mL) was stirred at refluxing for 3 hours, cooled to room temperature, filtered through a pad of Celite, extracted with ethyl acetate (2×20 mL). The combined organic phases were combined, dried over sodium sulfate and concentrated. Purification by column chromatography gave a final compound.

TABLE 13

| En | Structure | IUPAC Name | MW | Formula |
|---|---|---|---|---|
| 1 | 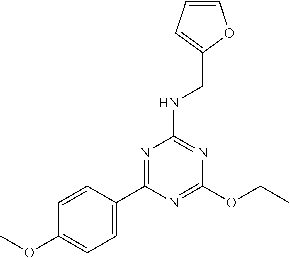 | 4-ethoxy-N-(furan-2-ylmethyl)-6-(4- methoxyphenyl)-1,3,5-triazin-2- amine | 326.4 | C17H16F3N5O2 |
| 2 | 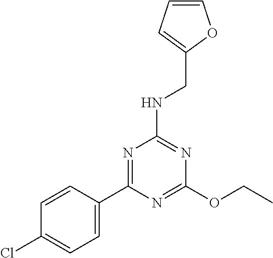 | 4-(4-chlorophenyl)-6-ethoxy-N- (furan-2-ylmethyl)-1,3,5-triazin-2- amine | 330.8 | C19H21N7O3 |
| 3 | 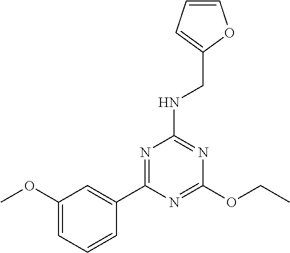 | 4-ethoxy-N-(furan-2-ylmethyl)-6-(3- methoxyphenyl)-1,3,5-triazin-2- amine | 326.3 | C20H22N6O4 |
| 4 | 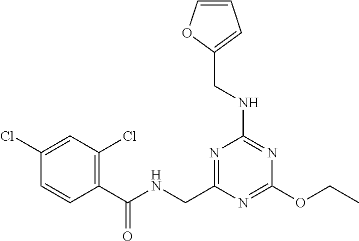 | 2,4-dichloro-N-({4-ethoxy-6-[(furan- 2-ylmethyl)amino]-1,3,5-triazin-2- yl}methyl)benzamide | 422.2 | C21H27N7O2 |

### Procedures and Analytical Data for Compounds in Table 13.

### 1. [4-Ethoxy-6-(4-methoxy-phenyl)-[1,3,5]triazin-2-yl]-furan-2-ylmethyl-amine

Yield 92 mg, 28%. ¹H-NMR (400 MHz, DMSO-D₆) δ_{H}: 1.32 (3H, t, J=7.5 Hz), 3.85 (3H, s), 4.40 (2H, superposition of two quartets, Z/E forms), 4.50-4.68 (2H, broad, Z/E forms), 6.25-6.33 (1H, broad Z/E forms), 6.88 (1H, broad), 7.03 (2H, d, J=8.5 Hz), 7.55 (1H, s), 8.15-8.22 (1H, broad, Z/E forms), 8.30 (2H, superposition of two doublets, Z/E forms). LCMS t_{R} (min): 1.97. MS (APCI), m/z 327.05 [M+H]⁺. HPLC t_{R} (min): 14.44. M_{P} 134-136° C.

### 2. [4-(4-Chloro-phenyl)-6-ethoxy-[1,3,5]triazin-2-yl]-furan-2-ylmethyl-amine

Yield 22 mg, 7%. ¹H-NMR (400 MHz, DMSO-D₆) δ_{H}: 1.33 (3H, broad triplet, J=7.5 Hz), 4.41 (2H, superposition of two quartets, J=7.5 Hz, Z/E forms), 4.50-4.69 (2H, two broad d, J=7.5 Hz, Z/E forms), 6.28-6.34 (1H, broad, Z/E forms), 6.40 (1H, broad), 7.52 (1H, s), 7.58 (2H, d, J=8.5 Hz), 8.25-8.39 (2H, two broad doublets, J=8.5 Hz, Z/E forms), 8.30-8.50 (1H, broad, Z/E forms). LCMS t_{R} (min): 2.15. MS (APCI), m/z 331.01, 332.99 [M+H]⁺. HPLC t_{R} (min): 16.53.

### 3. [4-Ethoxy-6-(3-methoxy-phenyl)-[1,3,5]triazin-2-yl]-furan-2-ylmethyl-amine

Yield 93 mg, 28%. ¹H-NMR (400 MHz, DMSO-D₆) δ_{H}: 1.32 (3H, t, J=7.5 Hz), 3.84 (3H, s), 4.40 (2H, superposition of two quartets, Z/E forms), 4.50-4.68 (2H, broad, Z/E forms), 6.30 (1H, broad), 6.40 (1H, broad), 7.15 (1H, broad doublet, J=8.5 Hz), 7.42 (1H, broad triplet, J=8.5 Hz), 7.55 (1H, s), 7.80-7.87 (1H, broad, Z/E forms), 7.87-8.00 (1H, two doublets, J=8.5 Hz, Z/E forms), 8.25-8.47 (1H, broad, Z/E forms). LCMS t_{R} (min): 1.97. MS (APCI), m/z 327.04 [M+H]⁺. HPLC t_{R} (min): 14.94. M_{P} 129-131° C.

### 4. 2,4-Dichloro-N-{4-ethoxy-6-[(furan-2-ylmethyl)-amino]-[1,3,5]triazin-2-ylmethyl}-benzamide

Preparation of Methylamine Derivatives in the Following Scheme was to convert intermediate 2 to cyano-derivative by the reaction of 2 with sodium cyanide in DMSO at 60° C. The cyano group was further reduced by LAH in THF at −30° C. to provide amine intermediate. Acylation of the amine by various acyl chloride to provide the desired amides.

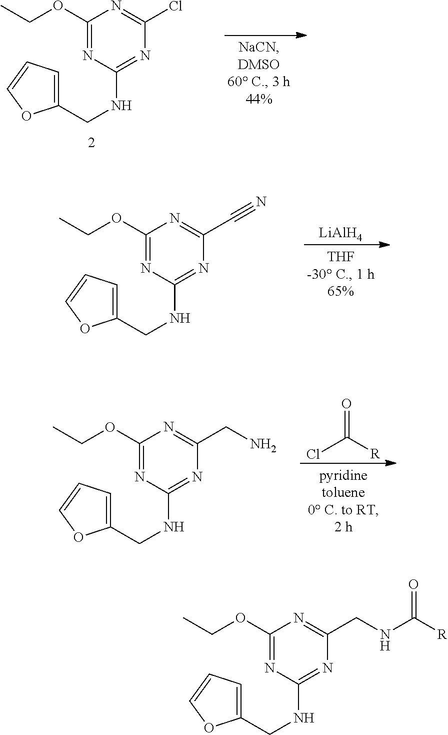

A mixture of compound 2 (1.200 g, 4.71 mmol), NaCN (1.155 g, 23.56 mmol) and DMSO (12 mL) was stirred at 60° C. for 4 hours, cooled to room temperature, diluted with water and extracted with dichloromethane. The combined organic phases were washed with brine, dried over sodium sulfate and purified by column chromatography (silica gel, dichloromethane) giving compound Ethoxy-6-[(furan-2-ylmethyl)-amino]-[1,3,5]-triazine-2-carbonitrile. Yield 503 mg, 44%.

Ethoxy-6-[(furan-2-ylmethyl)-amino-]-[1,3,5]triazine-2-carbonitrile (500 mg, 2.03 mmol) was added portionwise to a suspension of LiAlH₄ (387 mg, 10.19 mmol) in THF (12 mL) at −35° C. The mixture was stirred at the same temperature for 1 hour and then was let warm up slowly. Ethanol (6 mL) was added dropwise, when internal temperature of the mixture was −10° C. Then 15% aqueous KOH solution (50 mL) was added to the reaction mixture. The formed solid was filtered off and washed with ethyl acetate. The combined solutions were washed with water, brine, dried over sodium sulfate, concentrated at reduced pressure and dried in vacuum giving (4-Aminomethyl-6-ethoxy-[1,3,5]-triazin-2-yl)-furan-2-ylmethyl-amine. Yield 329 mg, 65%.

A solution of 2,4-dichloro-benzoyl chloride (0.18 mL, 1.28 mmol) in toluene (2 mL) was added to a solution of (4-Aminomethyl-6-ethoxy-[1,3,5]triazin-2-yl)-furan-2-ylmethyl-amine (320 mg, 1.28 mmol) at 0° C. during 1 hour. Then the obtained solution was stirred at 0° C. for 1 hour and at room temperature for 1 hour. 6% aqueous HCl solution was added the reaction mixture to reach pH 3. The mixture was extracted with ethyl acetate. The combined organic phases were washed with brine, dried over sodium sulfate and concentrated. The residue was purified by column chromatography on silica gel to provide the final compound. Yield 133 mg, 24%. ¹H-NMR (400 MHz, DMSO-D₆) δ_{H}: 1.22 (3H, broad), 4.07 (2H, broad), 4.23 (2H, broad), 4.42 (2H, broad), 6.20 (1H, broad), 6.20-6.40 (1H, broad, Z/E forms), 7.40 (1H, d, J=8.5 Hz), 7.43 (1H, broad), 7.52 (1H, broad), 7.58 (1H, broad), 7.62 (1H, broad), 7.90 (1H, broad), 9.40 (1H, broad). LCMS t_{R} (min): 1.86. MS (APCI), m/z 437.00, 439.00 [M+H]⁺. HPLC t_{R} (min): 12.38. M_{P} 166-168° C.

### Preparation of O,O,N-Triazines in Table 14

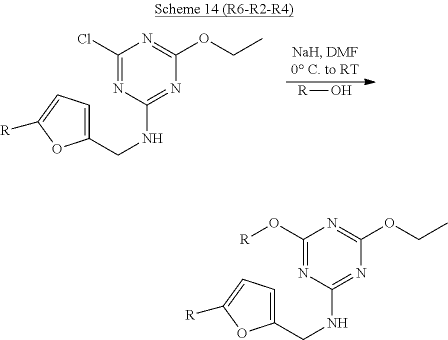

Derivatization of the common intermediate I-2 with O-nucleophiles (phenols, benzylic alcohols, alcohols) readily underwent under basic conditions. The reaction of I-2 with phenols proceeded in the presence of K₂CO₃ in acetonitrile. For benzyl alcohols, using of stronger base (NaH) is necessary. Conversion rate of this reaction was high.

TABLE 14

| En | Structure | IUPAC Name | MW | Formula |
|---|---|---|---|---|
| 1 | 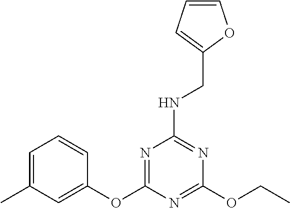 | 4-ethoxy-N-(furan-2-ylmethyl)-6- (3-methylphenoxy)-1,3,5-triazin- 2-amine | 326.4 | C18H21N5O4 |
| 2 | 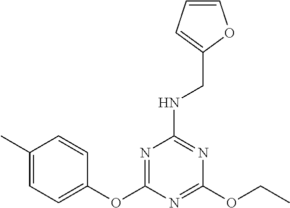 | 4-ethoxy-N-(furan-2-ylmethyl)-6- (4-methylphenoxy)-1,3,5-triazin- 2-amine | 326.4 | C17H18N4O3 |
| 3 | 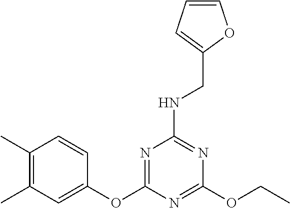 | 4-(3,4-dimethylphenoxy)-6- ethoxy-N-(furan-2-ylmethyl)- 1,3,5-triazin-2-amine | 340.4 | C18H19N5O4 |
| 4 | 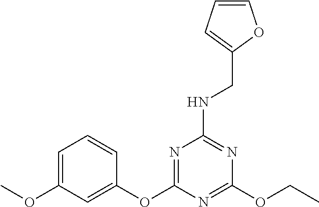 | 4-ethoxy-N-(furan-2-ylmethyl)-6- (3-methoxyphenoxy)-1,3,5- triazin-2-amine | 342.4 | C17H18N4O3 |
| 5 | 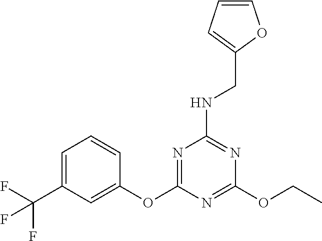 | 4-ethoxy-N-(furan-2-ylmethyl)-6- [3-(trifluoromethyl)phenoxy]- 1,3,5-triazin-2-amine | 380.3 | C17H18N4O4 |
| 6 | 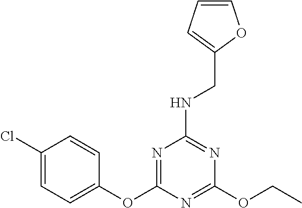 | 4-(4-chlorophenoxy)-6-ethoxy-N- (furan-2-ylmethyl)-1,3,5-triazin-2- amine | 346.8 | C18H20N4O3 |
| 7 | 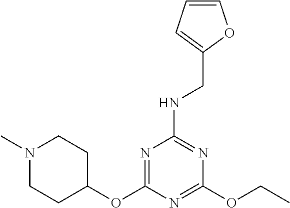 | 4-ethoxy-N-(furan-2-ylmethyl)-6- [(1-methylpiperidin-4-yl)oxy]- 1,3,5-triazin-2-amine | 333.4 | C18H17N7O2 |

### Procedures and Analytical Data for Compounds in Table 14.

### 1. (4-Ethoxy-6-m-tolyloxy-[1,3,5]triazin-2-yl)-furan-2-ylmethyl-amine

¹H-NMR (400 MHz, DMSO-D₆) δ_{H}: 1.20-1.28 (3H, m, Z/E forms), 2.30 (3H, s), 4.20-4.45 (2H, m, Z/E forms), 4.25-4.50 (2H, d, J=7.5 Hz, Z/E forms), 6.00-6.25 (1H, broad, Z/E forms), 6.35 (1H, broad, Z/E forms), 6.90-7.02 (2H, broad, Z/E forms), 7.06 (1H, broad), 7.28 (1H, broad), 7.53 (1H, d, J=1.8 Hz), 8.33 (1H, broad). LCMS t_{R} 1.94 (min). MS (APCI), m/z 326.76 [M+H]⁺. Mₚ 110-112° C.

### 2. (4-Ethoxy-6-p-tolyloxy-[1,3,5]triazin-2-yl)-furan-2-ylmethyl-amine

¹H-NMR (400 MHz, DMSO-D₆) δ_{H}: 1.20-1.28 (3H, m, Z/E forms), 2.30 (3H, s), 4.20-4.40 (2H, m, Z/E forms), 4.20-4.48 (2H, d, J=7.5 Hz, Z/E forms), 6.05-6.25 (1H, broad, Z/E forms), 6.36 (1H, broad, Z/E forms), 7.04 (2H, broad), 7.18 (2H, broad), 7.52 (1H, d, J=1.8 Hz), 8.28 (1H, t, J=7.5 Hz). LCMS t_{R} 1.93 (min). MS (APCI), m/z 326.79 [M+H]⁺. Mₚ 146-148° C.

### 3. [4-(3,4-Dimethyl-phenoxy)-6-ethoxy-[1,3,5]triazin-2-yl]-furan-2-ylmethyl-amine

¹H-NMR (400 MHz, DMSO-D₆) δ_{H}: 1.24 (3H, t, J=7.5 Hz), 2.20 (6H, s), 4.20-4.31 (2H, m, Z/E forms), 4.30-4.46 (2H, d, J=7.5 Hz, Z/E forms), 6.05-6.25 (1H, broad, Z/E forms), 6.37 (1H, broad, Z/E forms), 6.87 (1H, broad, Z/E forms), 6.94 (1H, broad, Z/E forms), 7.13 (1H, d, J=8.5 Hz), 7.52 (1H, s), 8.31 (1H, broad, Z/E forms). LCMS t_{R} 1.95 (min). MS (APCI), m/z 340.99 [M+H]⁺. Mₚ 110-112° C.

### 4. [4-(3,4-Dimethyl-phenoxy)-6-ethoxy-[1,3,5]triazin-2-yl]-furan-2-ylmethyl-amine

¹H-NMR (400 MHz, DMSO-D₆) δ_{H}: 1.20-1.30 (3H, m, Z/E forms), 3.72 (3H, s), 4.20-4.40 (2H, m, Z/E forms), 4.20-4.46 (2H, d, J=7.5 Hz, Z/E forms), 6.03-6.25 (1H, broad, Z/E forms), 6.30-6.38 (1H, broad, Z/E forms), 6.70-6.86 (3H, m), 7.29 (1H, t, J=8.5 Hz), 7.52 (1H, d, J=1.8 Hz), 8.34 (1H, t, J=7.5 Hz). LCMS t_{R} 1.86 (min). MS (APCI), m/z 342.77 [M+H]⁺. Mₚ 165-167° C.

### 5. [4-Ethoxy-6-(3-trifluoromethyl-phenoxy)-[1,3,5]triazin-2-yl]-furan-2-ylmethyl-amine

Sodium hydride (60% in oil, 24 mg, 0.6 mmol) was added portionwise to a solution of phenol (97 mg, 0.6 mmol) in DMF (5 mL) at 0° C. The obtained mixture was stirred at 0° for 5 minutes. Then 2 (128 mg, 0.5 mmol) was added portionwise at 0° C. The resulting mixture was stirred at 0° C. for 30 minutes and for 2 hours at room temperature, diluted with water (30 mL). The formed solid was collected by filtration and purified by column chromatography (silica gel, ethyl acetate/hexane) to give the product (50 mg, 26%). ¹H-NMR (400 MHz, DMSO-D₆) δ_{H}: 1.20-1.30 (3H, m, Z/E forms), 4.20-4.40 (2H, m, Z/E forms), 4.20-4.50 (2H, d, J=7.5 Hz, Z/E forms), 6.00-6.27 (1H, broad, Z/E forms), 6.30-6.40 (1H, broad, Z/E forms), 6.48-6.70 (5H, m), 8.38 (1H, broad). LCMS t_{R} 2.91 (min). MS (APCI), m/z 380.90 [M+H]⁺. Mₚ 97-99° C.

### 6. [4-(4-Chloro-phenoxy)-6-ethoxy-[1,3,5]triazin-2-yl]-furan-2-ylmethyl-amine

¹H-NMR (400 MHz, DMSO-D₆) δ_{H}: 1.20-1.30 (3H, m, Z/E forms), 4.20-4.31 (2H, m, Z/E forms), 4.31-4.47 (2H, d, J=7.5 Hz, Z/E forms), 6.05-6.25 (1H, broad, Z/E forms), 6.37 (1H, broad, Z/E forms), 7.22 (2H, d, J=8.5 Hz), 7.45 (2H, d, J=8.5 Hz), 7.52 (1H, s), 8.38 (1H, broad, Z/E forms). LCMS t_{R} 1.92 (min). MS (ARCI), m/z 346.94 [M+H]⁺. Mₚ 166-168° C.

### 7. [4-Ethoxy-6-(1-methyl-piperidin-4-yloxy)-[1,3,5]-triazin-2-yl]-furan-2-ylmethyl-amine

A solution of 1-methyl-piperidin-4-ol (276 mg, 2.4 mmol) in THF (1 mL) was added to a suspension of sodium hydride (60% in oil, 96 mg, 2.4 mmol) in THF (3 mL). The mixture was stirred at room temperature for 30 minutes. A solution of -(Furan-2-ylmethyl-amino)-4-Chloro-6-ethoxy-[1,3,5]triazine (300 mg, 1.18 mmol) in THF (2 mL) was added to the obtained mixture and the resulting mixture was stirred at refluxing for 2 hours, cooled to room temperature, poured into water (30 mL) and extracted with chloroform. The combined organic phases were washed with water, dried over sodium sulfate and concentrated. The residue was purified by column chromatography on silica gel (dichloromethane/methanol) and triturated with hexane giving a final compound.

Yield 80 mg, 20%. ¹H-NMR (400 MHz, DMSO-D₆) δ_{H}: 1.28 (3H, broad triplet, J=7.5 Hz), 1.63 (2H, broad), 1.90 (2H, broad), 2.18 (5H, m), 2.62 (2H, broad), 4.29 (2H, broad quartet, J=7.5 Hz), 4.46 (2H, broad), 4.90 (1H, broad), 6.22 (1H, broad), 6.38 (1H, broad), 7.52 (1H, s), 8.12 (1H, broad). LCMS t_{R} (min): 1.37. MS (APCI), m/z 333.94 [M+H]⁺. HPLC t_{R} (min): 8.41. M_{P} 192-194° C.

### Preparation O,N,N-Triazines in Table 15

There are two approaches to make this table of compounds: R6-R2-R4 route and R6-R4-R2 route.

General R6-R2-R4 method shown in the following Scheme is suitable for synthesis of R4 libraries. Having a common intermediate with R6 and R2 substituents a varied R4 fragment could be introduced on the last stage. Using R6-R2-R4 method based on the common intermediate, compounds with aryl amino moieties were synthesized in 23-63% yield, but treatment of the reactions and purification of products were convenient for fast synthesis of the final targets. Fragments of aliphatic amines were introduced at basic conditions with 20-38% yields.

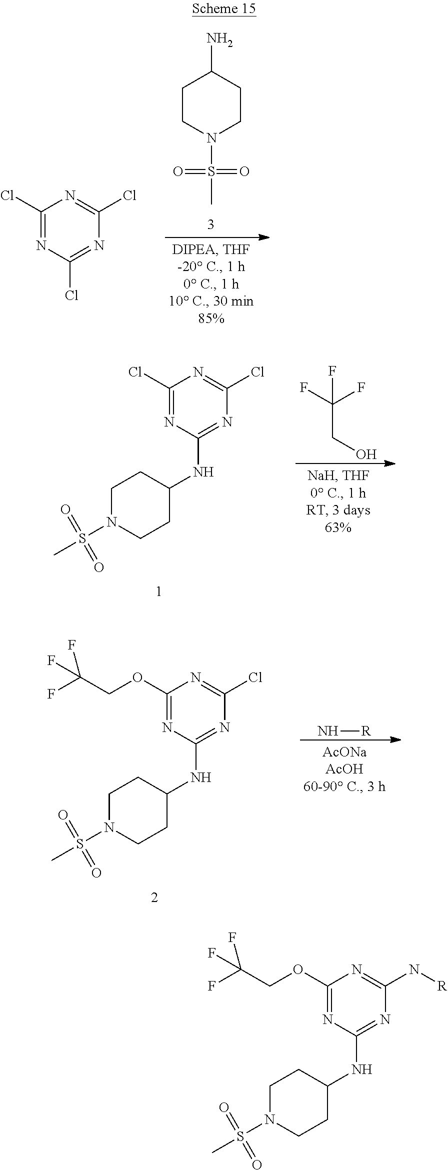

### (4,6-Dichloro-[1,3,5]triazin-2-yl)-(1-methanesulfonyl-piperidin-4-yl)-amine (1)

To a solution of cyanuric chloride (4.50 g, 24.13 mmol) in THF (50 mL) a suspension of 1-methanesulfonyl-piperidin-4-ylamine hydrochloride (3) (10.40 g, 48.26 mmol) and DIPEA (8 mL, 48.26 mmol) in THF (50 mL) was added slowly in portions at −20° C. The resulting mixture was stirred at −20° C. for 1 hour, warmed up to 0° C. and stirred for 1 hour at 0° C. and for 30 minutes at 10° C. Then, the reaction mixture was concentrated at reduced pressure. The residue was dissolved in dichloromethane and washed with water. The organic layer was dried over Na₂SO₄, filtered and concentrated. Purification by crystallization with a mixture of acetonitrile and hexane (1/1) gave compound 1 as yellow crystalline solid (3.84 g, 49%). The concentrated mother liquid (3.3 g) was purified by column chromatography on silica gel (DCM/ethyl acetate, 5/1) gave the additional portion of the compound 1 (2.80 g, 36%). Yield 6.64 g, 85%. MW 326.21. LCMS t_{R} (min): 1.53. MS (APCI+), m/z 326, 328, 330 [M+H]⁺.

### [4-Chloro-6-(2,2,2-trifluoro-ethoxy)-[1,3,5]triazin-2-yl]-(1-methanesulfonyl-piperidin-4-yl)-amine (2)

To a suspension of NaH (60% in oil, 100 mg, 2.5 mmol) in THF (5 mL) 2,2,2-trifluoroethanol (0.2 mL, 270 mg, 2.69 mmol) was added under stirring, and the resulting mixture was stirred for 15 minutes. Then, a solution of compound 1 (800 mg, 2.45 mmol) in THF (10 mL) was added to the mixture dropwise. The obtained mixture was stirred at 0° C. for 1 hour and at room temperature for 3 days, concentrated, diluted with dichloromethane and washed with water. The organic layer was dried over sodium sulfate and concentrated. Purification by column chromatography on silica gel (ethyl acetate/hexane) gave compound 2 as white crystals. Yield 600 mg, 63%.

A mixture of compound 2 (200 mg, 0.51 mmol), aniline (0.56 mmol), sodium acetate (152 mg, 1.12 mmol), acetic aside (2 mL) was stirred at 70° C. for 3 hours, cooled down to room temperature and diluted with water. The formed precipitate was filtered off and washed with water. Purification by column chromatography on silica gel or by recrystallization gave a final compound.

To a solution of compound 2 (260 mg, 0.66 mmol) in CH₃CN (5 mL) a solution of DIPEA (0.12 mL, 90 mg, 0.73 mmol) and amine (0.73 mmol) in CH₃CN (3 mL) was added dropwise at 0° C. Then, the mixture was allowed to warm up to 20° C., and stirred at that temperature for 2 hours. The reaction was monitored by TLC. Then, the mixture was concentrated. The residue was diluted with CH₂Cl₂, washed with water and brine, dried over Na₂SO₄ and concentrated. The residue was purified by column chromatography on silica gel or by recrystallization that gave a final compound.

1-Methanesulfonyl-piperidin-4-ylamine (3) was prepared via two paths. The paths had the last common stage which was performed in iso-propanol media and in dioxane media as well. The path starting from commercially available mono-protected diamine 5 was more efficient in terms of total yield after two steps (89%).

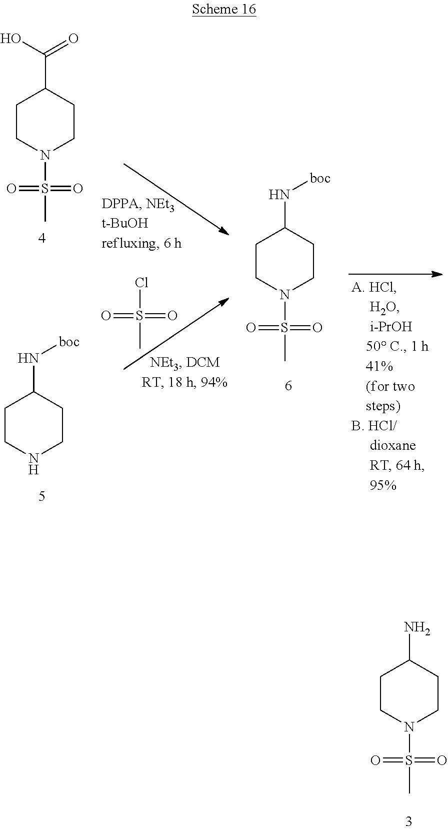

### 1-Methanesulfonyl-piperidin-4-ylamine hydrochloride (3)

Method A To a suspension of 1-methanesulfonyl-piperidine-4-carboxylic acid (2.5 g, 12.1 mmol) in t-butanol (30 mL) NEt₃ (1.46 g, 14.4 mmol) was added. The mixture was stirred for 10 minutes and DPPA (3.73 g, 13.3 mmol) was added. The resulting mixture was stirred at refluxing for 6 hours (TLC control), cooled down to room temperature and diluted with water. The obtained mixture was extracted with chloroform. The combined organic layers were washed with 40% K₂CO₃ aqueous solution, dried over Na₂SO₄ and concentrated at reduced pressure. The obtained residue was purified by column chromatography (silica gel, 5% ethanol/chloroform) and the solvent was evaporated on half. The formed precipitate was collected by filtration and washed with cold ether giving compound 4 used on the next stage.

To a solution of compound 4 in iso-propanol (40 mL) 6N HCl solution in iso-propanol (20 mL) was added at 60° C. The mixture was stirred at 50° C. for 1 hour, cooled down to room temperature and concentrated. The formed solid was collected by filtration and washed with ether giving compound 3 as hydrochloride. Yield 1.05 g, 41%.

Method B To a solution of N-Boc-piperidin-4-ylamine (7.39 g, 36.9 mmol) and NEt₃ (11.2 g, 110.7 mmol) in anhydrous dichloromethane (50 mL) a solution of methylsulfonyl chloride (4.44 g, 38.7 mmol) in dichloromethane (22 mL) was added at room temperature. The resulting mixture was stirred at room temperature for 18 hours. The formed precipitate was filtered off, washed with hexane and dried furnishing compound 4 as white crystalline solid. Yield 9.68 g, 94%. Compound 4 (9.68 g, 34.8 mmol) was dissolved in 16% HCl/dioxane (140 mL) and stirred at room temperature for 64 hours. The formed solid was collected by filtration, washed with hexane and dried giving compound 3 as hydrochloride as white crystals. Yield 7.09 g, 95%.

General R4-R6-R2 approach in the following Scheme was started with reaction of cyanuric chloride with anilines. The reactions were performed in presence of an organic base and without any base as well. In both cases low temperature was applied to avoid formation of bis-substituted byproducts. Substitution of the second chlorine atom in the triazine core by the 1-methanesulfonyl-piperidin-4-ylamine fragment was carried out at room temperature. Reactions with 2,2,2-trifluoro-ethanol were performed in presence of potassium carbonate and DMSO as a solvent. Generally this approach yields more than 50%

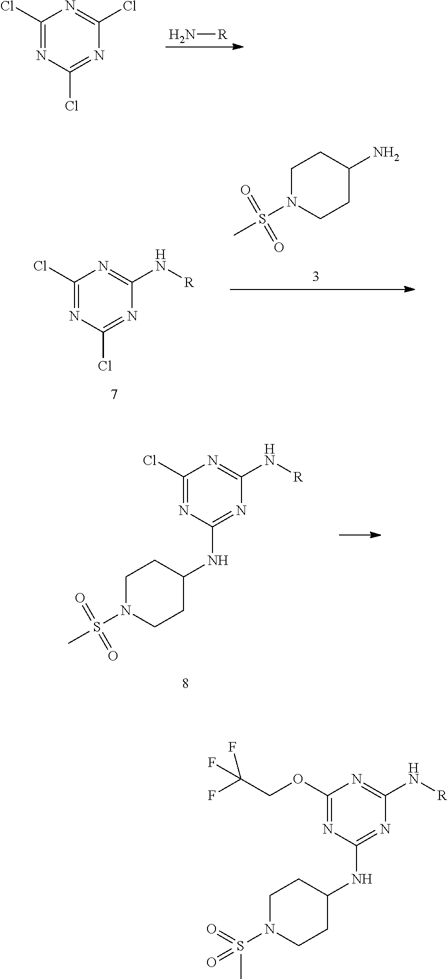

### N-(1-Methanesulfonyl-piperidin-4-yl)-6-chloro-N′-(3-trifluoromethyl-phenyl)-[1,3,5]triazine-2,4-diamine (8)

A mixture of compound 7 (618 mg, 2.00 mmol), 1-methanesulfonyl-piperidin-4-ylamine hydrochloride 3 as hydrochloride (430 mg, 2.00 mmol), DIPEA (645 mg, 5.00 mmol) and CH₃CN (15.0 mL) was stirred at room temperature for 3 hours (TLC control) and diluted with water. The formed solid was collected by filtration. Recrystallization from CH₃CN/H₂O and washing with Et₂O/hexane (1/2) gave the desired compound as white powder. Yield 520 mg, 58%. MW 450.87. LCMS t_{R} (min): 1.82. MS (APCI+), m/z 451.09, 453.06 [M+H]⁺. HPLC t_{R} (min): 14.69 (purity 93.86% (220 nm), 94.90% (254 nm). Impurity: HPLC t_{R} (min) 10.26 (6.14% (220 nm), 5.10% (254 nm)). M_{P} 220-222° C.

TABLE 15

| En | Structure | IUPAC Name | MW | Formula |
|---|---|---|---|---|
| 1 | 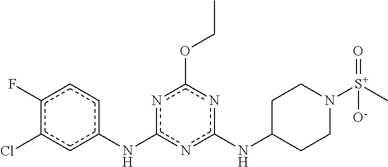 | N-(3-chloro-4-fluorophenyl)- 6-ethoxy-N′-[1- (methylsulfonyl)piperidin-4- yl]-1,3,5-triazine-2,4-diamine | 444.9 | C17H22ClFN6O3S |
| 2 | 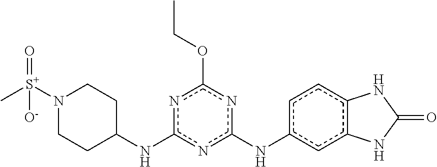 | 5-[(4-ethoxy-6-{[1- (methylsulfonyl)piperidin-4- yl]amino}-1,3,5-triazin-2- yl)amino]-1,3-dihydro-2H- benzimidazol-2-one | 448.5 | C18H24N8O4S |
| 3 | 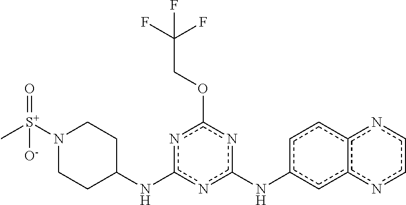 | N-[1- (methylsulfonyl)piperidin-4- yl]-N′-(quinoxalin-6-yl)-6- (2,2,2-trifluoroethoxy)-1,3,5- triazine-2,4-diamine | 498.5 | C19H21F3N8O3S |
| 4 | 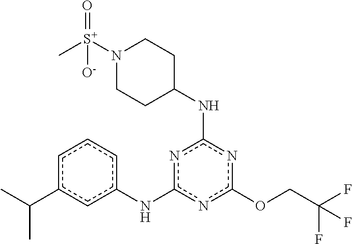 | N-[1- (methylsulfonyl)piperidin-4- yl]-N′-[3-(propan-2- yl)phenyl]-6-(2,2,2- trifluoroethoxy)-1,3,5- triazine-2,4-diamine | 488.5 | C20H27F3N6O3S |
| 5 | 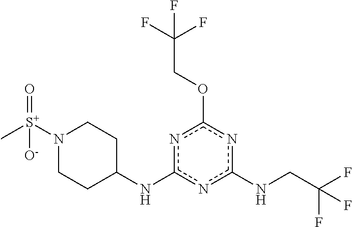 | N-[1- (methylsulfonyl)piperidin-4- yl]-6-(2,2,2-trifluoroethoxy)- N′-(2,2,2-trifluoroethyl)-1,3,5- triazine-2,4-diamine | 452.4 | C13H18F6N6O3S |
| 6 | 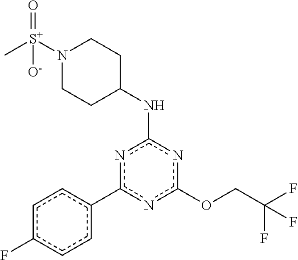 | 4-(4-fluorophenyl)-N-[1- (methylsulfonyl)piperidin-4- yl]-6-(2,2,2-trifluoroethoxy)- 1,3,5-triazin-2-amine | 449.4 | C17H19F4N5O3S |
| 7 | 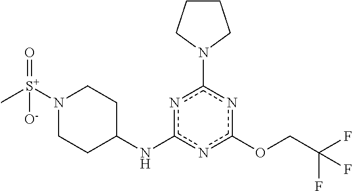 | N-[1- (methylsulfonyl)piperidin-4- yl]-4-(pyrrolidin-1-yl)-6- (2,2,2-trifluoroethoxy)-1,3,5- triazin-2-amine | 424.4 | C15H23F3N6O3S |
| 8 | 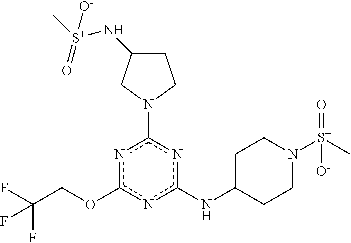 | N-{1-[4-{[1- (methylsulfonyl)piperidin-4-yl]- amino}-6-(2,2,2- trifluoroethoxy)-1,3,5-triazin-2- yl]pyrrolidin-3- yl}methanesulfonamide | 517.5 | C16H26F3N7O5S2 |
| 9 | 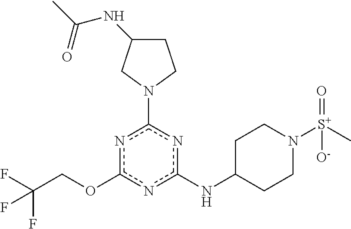 | N-{1-[4-{[1- (methylsulfonyl)piperidin-4- yl]amino}-6-(2,2,2- trifluoroethoxy)-1,3,5-triazin- 2-yl]pyrrolidin-3-yl}acetamide | 481.5 | C17H26F3N7O4S |
| 10 | 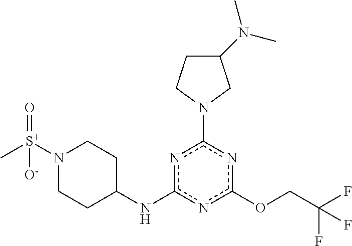 | 4-[3- (dimethylamino)pyrrolidin-1- yl]-N-[1- (methylsulfonyl)piperidin-4- yl]-6-(2,2,2-trifluoroethoxy)- 1,3,5-triazin-2-amine | 467.5 | C17H28F3N7O3S |
| 11 | 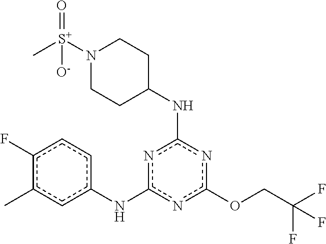 | N-(4-fluoro-3-methylphenyl)- N′-[1- (methylsulfonyl)piperidin-4- yl]-6-(2,2,2-trifluoroethoxy)- 1,3,5-triazine-2,4-diamine | 478.5 | C18H22F4N6O3S |
| 12 | 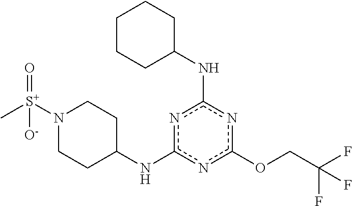 | N-cyclohexyl-N′-[1- (methylsulfonyl)piperidin-4- yl]-6-(2,2,2-trifluoroethoxy)- 1,3,5-triazine-2,4-diamine | 452.5 | C17H27F3N6O3S |
| 13 | 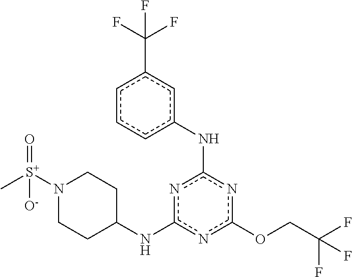 | N-[1- (methylsulfonyl)piperidin-4- yl]-6-(2,2,2-trifluoroethoxy)- N′-[3-(trifluoro- methyl)phenyl]-1,3,5-triazine- 2,4-diamine | 514.4 | C18H20F6N6O3S |
| 14 | 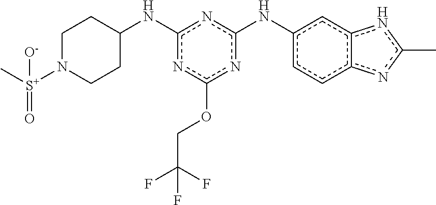 | N-(2-methyl-1H- benzimidazol-6-yl)-N′-[1- (methylsulfonyl)piperidin-4- yl]-6-(2,2,2-trifluoroethoxy)- 1,3,5-triazine-2,4-diamine | 500.5 | C19H23F3N8O3S |
| 15 | 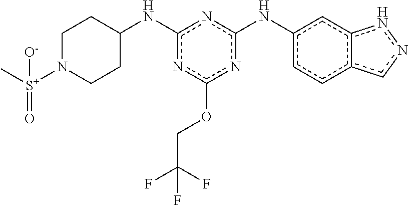 | N-(1H-indazol-6-yl)-N′-[1- (methylsulfonyl)piperidin-4- yl]-6-(2,2,2-trifluoroethoxy)- 1,3,5-triazine-2,4-diamine | 486.5 | C18H21F3N8O3S |
| 16 | 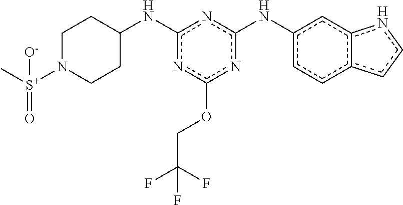 | N-(1H-indol-6-yl)-N′-[1- (methylsulfonyl)piperidin-4- yl]-6-(2,2,2-trifluoroethoxy)- 1,3,5-triazine-2,4-diamine | 485.5 | C19H22F3N7O3S |
| 17 | 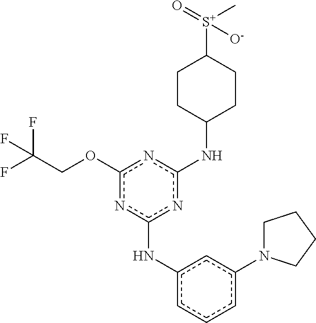 | N-[1- (methylsulfonyl)piperidin-4- yl]-N′-[3-(pyrrolidin-1- yl)phenyl]-6-(2,2,2- trifluoroethoxy)-1,3,5- triazine-2,4-diamine | 515.6 | C21H28F3N7O3S |
| 18 | 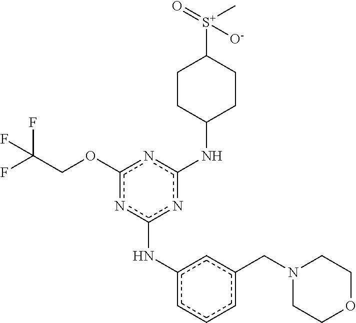 | N-[1- (methylsulfonyl)piperidin-4- yl]-N′-[3-(morpholin-4- ylmethyl)phenyl]-6-(2,2,2- trifluoroethoxy)-1,3,5- triazine-2,4-diamine | 545.6 | C22H30F3N7O4S |
| 19 | 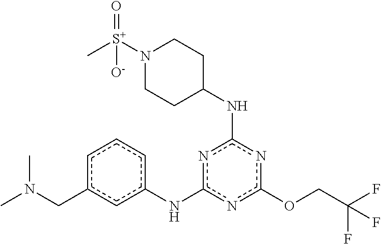 | N-{3- [(dimethylamino)methyl]phen- yl}-N′-[1- (methylsulfonyl)piperidin-4- yl]-6-(2,2,2-trifluoroethoxy)- 1,3,5-triazine-2,4-diamine | 503.5 | C20H28F3N7O3S |
| 20 | 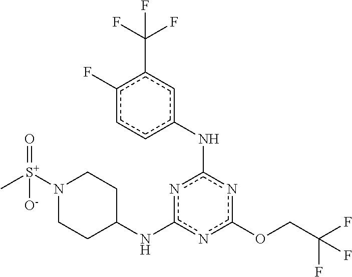 | N-[4-fluoro-3- (trifluoromethyl)phenyl]-N′-[1- (methylsulfonyl)piperidin-4- yl]-6-(2,2,2-trifluoroethoxy)- 1,3,5-triazine-2,4-diamine | 532.4 | C18H19F7N6O3S |
| 21 | 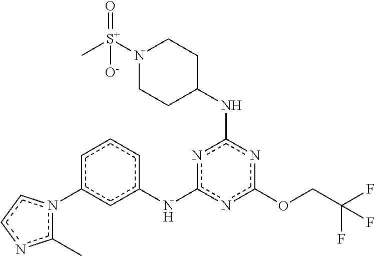 | 1,3,5-triazine-2,4-diamine, N2-[3-(2-methyl-1H-imidazol- 1-yl)phenyl]-N4-[1- (methylsulfonyl)-4- piperidinyl]-6-(2,2,2- trifluoroethoxy)- | 526.5 | C21H25F3N8O3S |
| 22 | 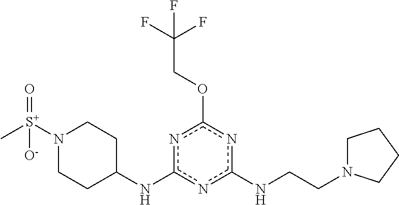 | N-[1- (methylsulfonyl)piperidin-4- yl]-N′-[2-(pyrrolidin-1- yl)ethyl]-6-(2,2,2- trifluoroethoxy)-1,3,5- triazine-2,4-diamine | 467.5 | C17H28F3N7O3S |
| 23 | 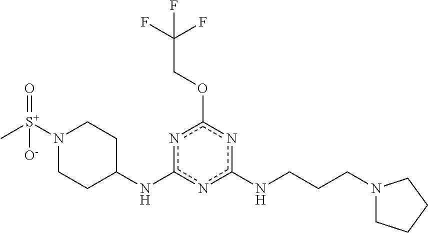 | N-[1- (methylsulfonyl)piperidin-4- yl]-N′-[3-(pyrrolidin-1- yl)propyl]-6-(2,2,2- trifluoroethoxy)-1,3,5- triazine-2,4-diamine | 481.5 | C18H30F3N7O3S |
| 24 | 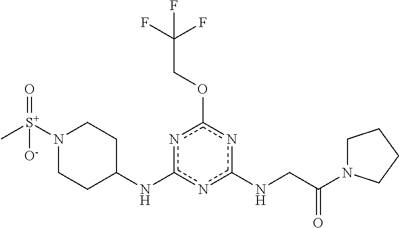 | 2-{[4-{[1- (methylsulfonyl)piperidin-4-yl]- amino}-6-(2,2,2- trifluoroethoxy)-1,3,5-triazin-2- yl]amino}-1-(pyrrolidin-1- yl)ethanone | 481.5 | C17H26F3N7O4S |
| 25 | 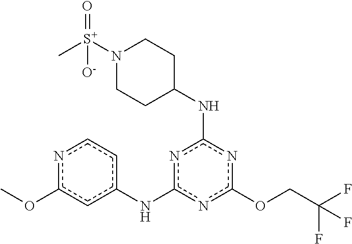 | N-(2-methoxypyridin-4-yl)-N′- [1-(methylsulfonyl)piperidin- 4-yl]-6-(2,2,2-trifluoroethoxy)- 1,3,5-triazine-2,4-diamine | 477.5 | C17H22F3N7O4S |
| 26 | 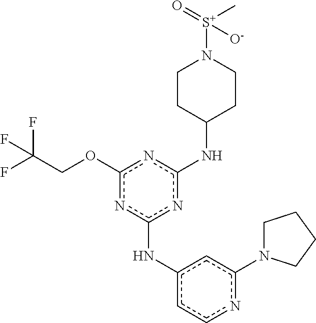 | N-[1- (methylsulfonyl)piperidin-4- yl]-N′-[2-(pyrrolidin-1- yl)pyridin-4-yl]-6-(2,2,2- trifluoroethoxy)-1,3,5- triazine-2,4-diamine | 516.5 | C20H27F3N8O3S |
| 27 | 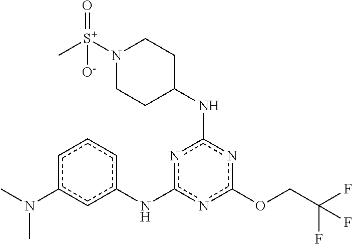 | N-[3-(dimethylamino)phenyl]- N′-[1- (methylsulfonyl)piperidin-4- yl]-6-(2,2,2-trifluoroethoxy)- 1,3,5-triazine-2,4-diamine | 489.5 | C19H26F3N7O3S |
| 28 | 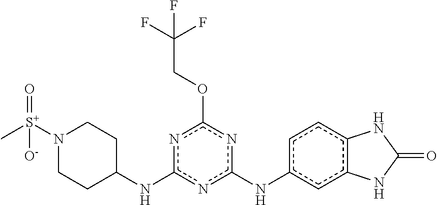 | 5-{[4-{[1- (methylsulfonyl)piperidin-4- yl]amino}-6-(2,2,2- trifluoroethoxy)-1,3,5-triazin-2- yl]amino}-1,3-dihydro-2H- benzimidazol-2-one | 502.5 | C18H21F3N8O4S |
| 29 | 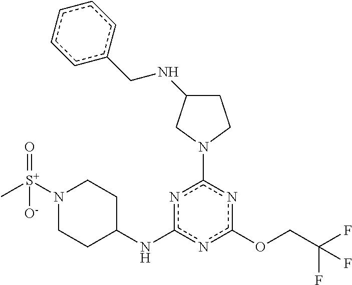 | 4-[3-(benzylamino)pyrrolidin- 1-yl]-N-[1- (methylsulfonyl)piperidin-4- yl]-6-(2,2,2-trifluoroethoxy)- 1,3,5-triazin-2-amine | 529.6 | C22H30F3N7O3S |
| 30 | 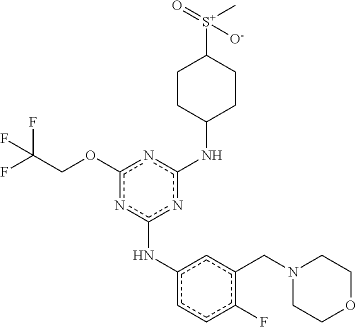 | N-[4-fluoro-3-(morpholin-4- ylmethyl)phenyl]-N′-[1- (methylsulfonyl)piperidin-4-yl]- 6-(2,2,2-trifluoroethoxy)-1,3,5- triazine-2,4-diamine | 563.6 | C22H29F4N7O4S |
| 31 | 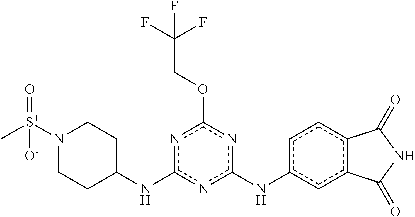 | 1H-isoindole-1,3(2H)-dione, 5- [[4-[[1-(methyl-sulfonyl)-4- piperidinyl]amino]-6-(2,2,2- trifluoroethoxy)-1,3,5-triazin-2- yl]amino]- | 515.5 | C19H20F3N7O5S |
| 32 | 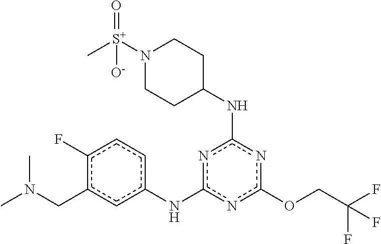 | N-{3-[(dimethylamino)methyl]- 4-fluorophenyl}-N′-[1- (methylsulfonyl)piperidin-4-yl]- 6-(2,2,2-trifluoroethoxy)-1,3,5- triazine-2,4-diamine | 521.5 | C20H27F4N7O3S |
| 33 | 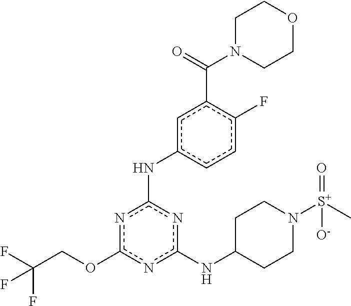 | methanone, [2-fluoro-5-[[4-[[1- (methylsulfonyl)-4- piperidinyl]amino]-6-(2,2,2- trifluoroethoxy)-1,3,5-triazin-2- yl]amino]phenyl]-4- morpholinyl- | 577.6 | C22H27F4N7O5S |
| 34 | 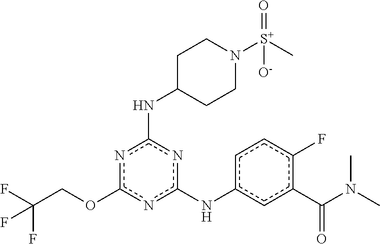 | benzamide, 2-fluoro-N,N- dimethyl-5-[[4-[[1- (methylsulfonyl)-4- piperidinyl]amino]-6-(2,2,2- trifluoroethoxy)-1,3,5-triazin-2- yl]amino]- | 535.5 | C20H25F4N7O4S |
| 35 | 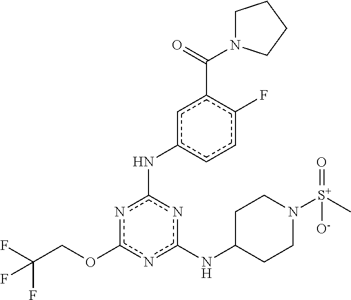 | (2-fluoro-5-{[4-{[1- (methylsulfonyl)-piperidin-4- yl]amino}-6-(2,2,2-trifluoro- ethoxy)-1,3,5-triazin-2- yl]amino}-phenyl)(pyrrolidin-1- yl)methanone | 561.6 | C22H27F4N7O4S |

### Procedures and Analytical Data for Compounds in Table 15.

### 1. N-(3-Chloro-4-fluoro-phenyl)-6-ethoxy-N′-(1-methanesulfonyl-piperidin-4-yl)-[1,3,5]triazine-2,4-diamine

Yield 163 mg, 73%. ¹H-NMR (400 MHz, DMSO-D₆) δ_{H}: 1.28 (3H, broad), 1.58 (2H, broad), 1.95 (2H, broad), 2.85 (5H, superposition of s and m), 3.59 (2H, broad), 3.90 (1H, broad), 4.30 (2H, broad q, J=7.5 Hz), 7.31 (1H, broad), 7.43-7.57 (1H, two broad peaks, Z/E forms), 7.57-7.70 (1H, two broad peaks, Z/E forms), 8.00-8.10 (1H, two broad peaks), 9.40-9.58 (1H, two broad peaks). MW 444.92. LCMS t_{R} (min): 1.82. MS (APCI), m/z 445.11 [M+H]⁺. HPLC t_{R} (min): 13.18. M_{P} 197-199° C.

### 2. 5-[4-Ethoxy-6-(1-methanesulfonyl-piperidin-4-ylamino)-[1,3,5]triazin-2-yl-amino]-1,3-dihydro-benzoimidazol-2-one

¹H-NMR (400 MHz, DMSO-D₆) δ_{H}: 1.25 (3H, broad), 1.57 (2H, m), 1.95 (2H, m), 2.87 (5H, m), 3.55 (2H, m), 3.90 (1H, broad peak), 4.30 (2H, broad q, J=7.5 Hz), 6.80 (1H, d, J=8.5 Hz, Z/E forms), 7.18 (1H, broad peak, Z/E forms), 7.30 (1H, broad peak, Z/E forms), 7.31-7.50 (1H, broad peak, Z/E forms), 9.00-9.20 (1H, broad peak, Z/E forms), 10.31 (1H, broad peak, Z/E forms), 10.32-10.50 (1H, broad peak, Z/E forms). MW 448.51. LCMS t_{R} (min): 1.4. MS (APCI), m/z 449.11 [M+H]⁺. HPLC t_{R} (min): 7.95. M_{P} 333° C. (dec).

### 3. N-(1-Methanesulfonyl-piperidin-4-yl)-N′-quinoxalin-6-yl-6-(2,2,2-trifluoro-ethoxy)-[1,3,5]triazine-2,4-diamine

Yield 130 mg, 41%. ¹H-NMR (400 MHz, DMSO-D₆) δ_{H}: 0.93 (3H, t, J=7.5 Hz), 1.58 (2H, m), 1.71 (2H, q, J=7.5 Hz), 1.94 (2H, m), 2.40 (2H, broad peak), 2.86 (11H, broad peak), 3.55 (2H, m), 3.98 (1H, broad peak, Z/E forms), 6.38 (1H, broad peak, Z/E forms), 7.08 (2H, broad peak, Z/E forms), 7.26 (1H, broad peak, Z/E forms), 7.27-7.42 (1H, broad peaks, Z/E forms), 8.98-9.12 (1H, broad peak, Z/E forms). MW 498.49. LCMS t_{R} (min): 1.68. MS (APCI), m/z 499.1 [M+H]⁺. HPLC t_{R} (min): 12.14. M_{P} 178-180°.

### 4. N-(3-Isopropyl-phenyl)-N′-(1-methanesulfonyl-piperidin-4-yl)-6-(2,2,2-trifluoro-ethoxy)-[1,3,5]triazine-2,4-diamine

Yield 580 mg, 89%. ¹H-NMR (400 MHz, DMSO-D₆) δ_{H}: 1.20 (6H, d, J=7.5 Hz), 1.60 (2H, broad), 1.93 (2H, broad), 2.85 (6H, broad m), 3.58 (2H, m), 3.95 (1H, broad peak, Z/E forms), 4.95 (2H, q, J=7.5 Hz), 6.90 (1H, d, J=8.5 Hz), 7.20 (1H, broad peak, Z/E forms), 7.45 (1H, broad peak, Z/E forms), 7.50-7.61 (1H, broad peaks, Z/E forms), 7.65 (1H, broad peak, Z/E forms), 9.30-9.50 (1H, broad peak, Z/E forms). MW 488.54. LCMS t_{R} (min): 2.01. MS (APCI), m/z 489.16 [M+H]⁺. HPLC t_{R} (min): 15.90. M_{P} 228-229° C.

### 5. N-(1-Methanesulfonyl-piperidin-4-yl)-6-(2,2,2-trifluoro-ethoxy)-N′-(2,2,2-trifluoro-ethyl)-[1,3,5]triazine-2,4-diamine

Yield 139 mg, 38%. ¹H-NMR (400 MHz, DMSO-D₆) δ_{H}: 1.55 (2H, m), 1.90 (2H, m), 2.85 (5H, m), 3.53 (2H, m), 3.90 (1H, broad peak, Z/E forms), 4.10 (2H, broad peak, Z/E forms), 4.90 (2H, broad peak, Z/E forms), 7.45-7.65 (1H, two broad peaks, Z/E forms), 7.76-7.99 (1H, two broad peaks, Z/E forms). MW 452.38. LCMS t_{R} (min): 1.71. MS (APCI+), m/z 453.09 [M+H]⁺. HPLC t_{R} (min): 13.56. M_{P} 216-218° C.

### 6. [4-(4-Fluoro-phenyl)-6-(2,2,2-trifluoro-ethoxy)-[1,3,5]triazin-2-yl]-(1-methanesulfonyl-piperidin-4-yl)-amine

Yield 160 mg, 52%. ¹H-NMR (400 MHz, DMSO-D₆) δ_{H}: 1.62 (2H, m), 2.00 (2H, m), 2.87 (3H, s), 2.95 (2H, m), 3.59 (2H, m), 4.00-4.18 (1H, two broad peaks, Z/E forms), 5.08 (2H, superposition of two q, J=7.5 Hz), 7.33 (2H, d/d, J=8.5/8.0 Hz, Z/E forms), 8.20-8.30 (1H, two broad peaks, Z/E forms), 8.42 (2H, superposition of two m, Z/E forms). MW 449.43. LCMS t_{R} (min): 1.91. MS (APCI+), m/z 450.11 [M+H]⁺. HPLC t_{R} (min): 15.72. M_{P} 221-223° C.

### 7. (1-Methanesulfonyl-piperidin-4-yl)-[4-pyrrolidin-1-yl-6-(2,2,2-trifluoro-ethoxy)-[1,3,5]triazin-2-yl]-amine

Yield 300 mg, 85%. ¹H-NMR (400 MHz, DMSO-D₆) δ_{H}: 1.63 (2H, m), 1.88 (6H, broad peak), 2.86 (5H, superposition of s and m), 3.42 (4H, broad peaks, Z/E forms), 3.52 (2H, m), 3.88 (1H, broad peak, Z/E forms), 4.89 (2H, broad q, J=7.5 Hz), 7.30 (1H, broad peak, Z/E forms). MW 424.44. LCMS t_{R} (min): 1.77. MS (APCI+), m/z 425.16 [M+H]⁺. HPLC t_{R} (min): 13.10. M_{P} 232-234° C.

### 8. N-{1-[4-(1-Methanesulfonyl-piperidin-4-ylamino)-6-(2,2,2-trifluoro-ethoxy)-[1,3,5]triazin-2-yl]-pyrrolidin-3-yl}-methanesulfonamide

Yield 60 mg, 29%. ¹H-NMR (400 MHz, DMSO-D₆) δ_{H}: 1.54 (2H, broad peak, Z/E forms), 1.90 (2H, broad peak, Z/E forms), 1.90-2.18 (2H, two broad peaks, Z/E forms), 2.86 (5H, m), 2.97 (3H, s), 3.37-3.46 (2H, broad peak, Z/E forms), 3.52 (2H, m), 3.52-3.60 (2H, two broad peaks, Z/E forms), 3.73 (1H, m), 3.93 (1H, broad peak, Z/E forms), 4.90 (2H, broad q, J=7.5 Hz), 7.30 (1H, d, J=7.5 Hz), 7.33-7.45 (1H, broad d, J=7.5 Hz, Z/E forms). MW 517.55. LCMS t_{R} (min): 1.55. MS (APCI+), m/z 518.16 [M+H]⁺. HPLC t_{R} (min): 10.85. M_{P} 118-120° C.

### 9. N-{1-[4-(1-Methanesulfonyl-piperidin-4-ylamino)-6-(2,2,2-trifluoro-ethoxy)-[1,3,5]triazin-2-yl]-pyrrolidin-3-yl}-acetamide

Yield 55 mg, 22%. ¹H-NMR (400 MHz, DMSO-D₆) δ_{H}: 1.53 (2H, broad peak, Z/E forms), 1.80 (3H, s), 1.90 (2H, broad peak, Z/E forms), 2.10 (2H, broad peak, Z/E forms), 2.85 (5H, superposition of s and m, Z/E forms), 3.26-3.38 (2H, two broad peaks, Z/E forms), 3.52 (2H, m), 3.52-3.90 (2H, two broad peaks, Z/E forms), 3.64 (1H, broad peak, Z/E forms), 4.27 (1H, broad peak, Z/E forms), 4.88 (2H, broad q, J=7.5 Hz), 7.34 (1H, broad peak, Z/E forms), 8.00 (1H, broad peak, Z/E forms). MW 481.50. LCMS t_{R} (min): 1.50. MS (APCI+), m/z 482.16 [M+H]⁺. HPLC t_{R} (min): 9.90. M_{P} 218-220° C.

### 10. [4-(3-Dimethylamino-pyrrolidin-1-O-6-(2,2,2-trifluoro-ethoxy)-[1,3,5]triazin-2-yl]-(1-methanesulfonyl-piperidin-4-yl)-amine

Yield 170 mg, 49%. ¹H-NMR (400 MHz, DMSO-D₆) δ_{H}: 1.53 (2H, m), 1.74-1.90 (2H, broad, Z/E forms), 1.90-2.08 (2H, broad peaks, Z/E forms), 2.19 (6H, s), 2.73 (2H, broad peak, Z/E forms), 2.87 (5H, superposition of s and m), 3.37-3.65 (2H, two broad peaks, Z/E forms), 3.52 (2H, m), 3.70-3.90 (2H, two broad peaks, Z/E forms), 4.90 (2H, broad q, J=7.5 Hz), 7.38 (1H, broad d, J=7.5 Hz, Z/E forms). MW 467.51. LCMS t_{R} (min): 1.33. MS (APCI+), m/z 468.21 [M+H]⁺. HPLC t_{R} (min): 8.79. M_{P} 190-192° C.

### 11. N-(4-Fluoro-3-methyl-phenyl)-N′-(1-methanesulfonyl-piperidin-4-yl)-6-(2,2,2-trifluoro-ethoxy)-[1,3,5]triazine-2,4-diamine

Yield 207 mg, 78%. ¹H-NMR (400 MHz, DMSO-D₆) δ_{H}: 1.60 (2H, m), 1.90-2.05 (2H, broad peak, Z/E forms), 2.22 (3H, s), 2.88 (5H, superposition of s and m), 3.60 (2H, m), 3.92 (1H, broad peak, Z/E forms), 4.95 (2H, broad q, J=7.5 Hz), 7.06 (1H, m), 7.48 (1H, broad peak, Z/E forms), 7.55 (1H, broad peak, Z/E forms), 7.60-7.70 (1H, two broad peaks, Z/E forms), 9.37-9.57 (1H, two broad peaks, Z/E forms). MW 478.47. LCMS t_{R} (min): 1.87. MS (APCI+), m/z 479.12 [M+H]⁺. HPLC t_{R} (min): 15.14. M_{P} 228-229° C.

### 12. N-Cyclohexyl-N′-(1-methanesulfonyl-piperidin-4-yl)-6-(2,2,2-trifluoro-ethoxy)-[1,3,5]triazine-2,4-diamine

Yield 159 mg, 50%. ¹H-NMR (400 MHz, DMSO-D₆) δ_{H}: 1.05-1.18 (4H, two broad peaks, Z/E forms), 1.25-1.55 (4H, broad peak, Z/E forms), 1.71 (2H, broad peak, Z/E forms), 1.82 (2H, broad peak, Z/E forms), 1.92 (2H, broad peak), 2.83 (5H, broad peak, Z/E forms), 3.52 (2H, m), 3.71 (1H, broad peak, Z/E forms), 3.88 (1H, broad peak, Z/E forms), 4.87 (2H, broad q, J=7.5 Hz), 7.07-7.20 (1H, two broad peaks, Z/E forms), 7.20-7.32 (1H, two broad peaks, Z/E forms). MW 452.50. LCMS t_{R} (min): 1.95. MS (APCI+), m/z 453.14 [M+H]⁺. HPLC t_{R} (min): 14.04. M_{P} 98-100° C.

### 13. N-(1-Methanesulfonyl-piperidin-4-yl)-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 1.61 (2H, m), 1.98 (2H, m), 2.79 (2H, m), 2.91 (3H, s), 3.60 (2H, m), 3.91 (1H, broad peak), 4.95 (2H, broad q, J=7.5 Hz), 7.31 (1H, broad d, J=8.5 Hz, Z/E forms), 7.51 (1H, superposition of two t, J=8.5 Hz, Z/E forms), 7.83 (1H, broad d, J=8.5 Hz, Z/E forms), 7.62-8.11 (1H, two broad peaks, Z/E forms), 8.01-8.31 (1H, broad s, Z/E forms), 9.71-10.01 (1H, two broad peaks, Z/E forms). MW 514.45. LCMS t_{R} (min): 1.92. MS (APCI+), m/z 515.12 [M+H]⁺. HPLC t_{R} (min): 15.94 (purity 99.98%).

### 14. N-(1-Methanesulfonyl-piperidin-4-yl)-N′-(2-methyl-3H-benzoimidazol-5-yl)-6-(2,2,2-trifluoro-ethoxy)-[1,3,5]triazine-2,4-diamine

¹H-NMR (400 MHz, DMSO-D₆) δ_{H}: 1.60 (2H, m), 2.00 (2H, m), 2.45 (3H, s), 2.90 (5H, superposition of m and s), 3.58 (2H, m), 3.95 (1H, broad peak), 4.45 (2H, broad q, J=7.5 Hz), 7.33 (2H, broad peaks), 7.50-7.61 (1H, two broad peaks, Z/E forms), 7.79-7.90 (1H, two broad peaks, Z/E forms), 9.30-9.50 (1H, two broad peaks, Z/E forms), 11.98 (1H, broad peak). MW 500.51. LCMS t_{R} (min): 1.44. MS (APCI+), m/z 501.16 [M+H]⁺. HPLC t_{R} (min): 9.49. M_{P} 268-272° C.

### 15. N-(1H-Indazol-6-yl)-N′-(1-methanesulfonyl-piperidin-4-yl)-6-(2,2,2-trifluoro-ethoxy)-[1,3,5]triazine-2,4-diamine

¹H-NMR (400 MHz, DMSO-D₆) δ_{H}: 1.60 (2H, m), 2.00 (2H, m), 2.90 (5H, superposition of s and m), 3.59 (2H, m), 3.98 (1H, broad peaks), 4.98 (2H, broad q, J=7.5 Hz), 7.30-7.42 (1H, broad d, J=8.5 Hz, Z/E forms), 7.62 (1H, broad peaks, Z/E forms), 7.62-7.72 (1H, two broad peaks, Z/E forms), 7.94 (1H, s), 7.94-8.10 (1H, two s, Z/E forms), 9.52-9.72 (1H, two broad peaks, Z/E forms), 12.72-12.82 (1H, two broad peaks, Z/E forms). MW 486.47. LCMS t_{R} (min): 1.63. MS (APCI+), m/z 487.13 [M+H]⁺. HPLC t_{R} (min): 11.96. M_{P} 233-234° C.

### 16. N-(1H-Indol-6-yl)-N′-(1-methanesulfonyl-piperidin-4-yl)-6-(2,2,2-trifluoro-ethoxy)-[1,3,5]-triazine-2,4-diamine

¹H-NMR (400 MHz, DMSO-D₆) δ_{H}: 1.60 (2H, m), 1.95 (2H, m), 2.88 (5H, superposition of m and s), 3.55 (2H, m), 3.95 (1H, broad peak), 4.95 (2H, broad qt, J=7.5 Hz), 6.32 (1H, s), 7.22 (2H, m), 7.40 (1H, s), 7.45-7.58 (1H, broad peaks, Z/E forms), 7.70-7.85 (1H, broad peaks, Z/E forms), 9.25-9.45 (1H, broad peaks, Z/E forms), 10.86 (1H, broad peak). MW 485.49. LCMS t_{R} (min): 1.78. MS (APCI+), m/z 486.11 [M+H]⁺. HPLC t_{R} (min): 13.68. M_{P} 240-242° C.

### 17. N-(1-Methanesulfonyl-piperidin-4-yl)-N′-(3-pyrrolidin-1-yl-phenyl)-6-(2,2,2-trifluoro-ethoxy)-[1,3,5]triazine-2,4-diamine

¹H-NMR (400 MHz, DMSO-D₆) δ_{H}: 1.58 (2H, m), 1.93 (6H, m), 2.83 (5H, superposition of s and m), 3.20 (4H, m), 3.45 (2H, m), 3.97 (1H, broad peak), 4.93 (2H, q, J=7.5 Hz), 6.22 (1H, d, J=8.5 Hz), 6.80-6.92 (1H, two broad peaks, Z/E forms), 7.02 (2H, broad peaks), 7.45-7.60 (1H, two broad peaks, Z/E forms), 9.14-9.33 (1H, two broad peaks, Z/E forms). MW 515.56. LCMS t_{R} (min): 1.94. MS (APCI+), m/z 516.18, 518.19 [M+H]⁺. HPLC t_{R} (min): 14.25. M_{P} 216-218° C.

### 18. N-(1-Methanesulfonyl-piperidin-4-yl)-N′-(3-morpholin-4-ylmethyl-phenyl)-6-(2,2,2-trifluoro-ethoxy)-[1,3,5]triazine-2,4-diamine

¹H-NMR (400 MHz, DMSO-D₆) δ_{H}: 1.60 (2H, m), 1.95 (2H, m), 2.38 (4H, m), 2.90 (2H, s), 3.45 (6H, m), 3.70 (5H, superposition of m and s), 3.95 (1H, broad peak, Z/E forms), 4.95 (2H, q, J=7.5 Hz), 6.96 (1H, d, J=8.5 Hz), 7.24 (1H, broad peak, Z/E forms), 7.52 (1H, broad peak), 7.52-7.70 (1H, two broad peaks, Z/E forms), 7.70 (1H, broad peak, Z/E forms), 9.38-9.60 (1H, two broad peaks, Z/E forms). MW 545.58. LCMS t_{R} (min): 1.46. MS (APCI+), m/z 546.12 [M+H]⁺. HPLC t_{R} (min): 9.86. M_{P} 289-290° C.

### 19. N-(3-Dimethylaminomethyl-phenyl)-N′-(1-methanesulfonyl-piperidin-4-yl)-6-(2,2,2-trifluoro-ethoxy)-[1,3,5]triazine-2,4-diamine

¹H-NMR (400 MHz, DMSO-D₆) δ_{H}: 1.62 (2H, m), 2.01 (2H, m), 2.15 (6H, s), 2.89 (5H, superposition of s (3H) and m (2H)), 3.48 (2H, s), 3.61 (2H, m), 3.95 (1H, broad peak, Z/E forms), 4.95 (2H, q, J=7.5 Hz), 6.91 (1H, broad peak, Z/E forms), 7.21 (1H, broad peak, Z/E forms), 7.58 (1H, broad peak, Z/E forms), 7.71 (2H, broad peak, Z/E forms), 9.38-9.65 (1H, two broad peaks, Z/E forms). MW 503.55. LCMS t_{R} (min): 1.43. MS (APCI+), m/z 504.12 [M+H]⁺. HPLC t_{R} (min): 9.82. M_{P} 115-117° C.

### 20. N-(4-Fluoro-3-trifluoromethyl-phenyl)-N′-(1-methanesulfonyl-piperidin-4-yl)-6-(2,2,2-trifluoro-ethoxy)-[1,3,5]triazine-2,4-diamine

Yield 90 mg, 33%. ¹H-NMR (400 MHz, DMSO-D₆) δ_{H}: 1.60 (2H, m), 1.95 (2H, m), 2.80 (2H, m), 2.89 (3H, s), 3.58 (2H, m), 3.89 (1H, broad), 4.95 (2H, broad q, J=7.5 Hz), 7.43 (1H, superposition of two m), 7.68-7.82 (1H, two broad peaks, Z/E forms), 7.88-8.02 (1H, two broad peaks, Z/E forms), 8.12-8.26 (1H, two broad peaks, Z/E forms), 9.71-9.92 (1H, two broad peaks, Z/E forms). MW 532.44. LCMS t_{R} (min): 1.94. MS (APCI+), m/z 533.12 [M+H]⁺. HPLC t_{R} (min): 16.02. M_{P} 210-212° C.

### 21. N-(1-Methanesulfonyl-piperidin-4-yl)-N′-[3-(2-methyl-imidazol-1-yl)-phenyl]-6-(2,2,2-trifluoro-ethoxy)-[1,3,5]triazine-2,4-diamine

MW 526.5. MS (APCI+), m/z 527.28 [M+H]⁺, 459.26 (imp.). HPLC t_{R} (min): 10.26.

### 22. N-(1-Methanesulfonyl-piperidin-4-yl)-N′-(2-pyrrolidin-1-yl-ethyl)-6-(2,2,2-trifluoro-ethoxy)-[1,3,5]triazine-2,4-diamine

¹H-NMR (400 MHz, DMSO-D₆) δ_{H}: 1.52 (2H, m), 1.67 (4H, m), 1.90 (2H, m), 2.51 (6H, m), 2.85 (5H, superposition of s and m), 3.35 (2H, broad peak, Z/E forms), 3.54 (2H, m), 3.90 (1H, broad peak, Z/E forms), 4.88 (2H, broad peak, Z/E forms), 7.00-7.20 (1H, two broad peaks, Z/E forms), 7.27-7.38 (1H, two broad peaks, Z/E forms). MW 467.52. LCMS t_{R} (min): 1.36. MS (APCI+), m/z 468.13 [M+H]⁺. HPLC t_{R} (min): 9.08. M_{P} 179-182° C.

### 23. N-(1-Methanesulfonyl-piperidin-4-yl)-N′-(3-pyrrolidin-1-yl-propyl)-6-(2,2,2-trifluoro-ethoxy)-[1,3,5]triazine-2,4-diamine

¹H-NMR (400 MHz, DMSO-D₆) δ_{H}: 1.58 (2H, broad peak, Z/E forms), 1.77 (2H, m), 1.89 (4H, m), 2.85 (5H, superposition of s and m), 3.12 (2H, m), 3.30 (2H, broad peak, Z/E forms), 3.35 (6H, broad peak, Z/E forms) 3.43 (2H, broad peak, Z/E forms), 3.52 (2H, broad peak, Z/E forms), 3.80-3.90 (1H, two broad peaks, Z/E forms), 4.00 (2H, broad peak, Z/E forms), 4.88 (2H, broad q, J=7.5 Hz), 6.92-7.40 (1H, two broad peaks, Z/E forms), 7.08-7.33 (1H, two broad peaks, Z/E forms). MW 481.54. LCMS t_{R} (min): 1.41. MS (APCI+), m/z 411.10 [M+H]⁺. HPLC t_{R} (min): 9.16. M_{P} 190-194° C.

### 24. 2-[4-(1-Methanesulfonyl-piperidin-4-ylamino)-6-(2,2,2-trifluoro-ethoxy)-[1,3,5]triazin-2-ylamino]-1-pyrrolidin-1-yl-ethanone

¹H-NMR (400 MHz, DMSO-D₆) δ_{H}: 1.54 (2H, m), 1.88 (4H, two broad peaks, Z/E forms), 2.06 (2H, broad peak, Z/E forms), 2.18 (2H, broad peak, Z/E forms), 2.88 (5H, superposition of s and m), 3.50 (2H, m), 3.90 (1H, broad peak, Z/E forms), 4.88 (2H, superposition of two q, J=7.5 Hz, Z/E forms), 7.28-7.40 (1H, two broad peaks, Z/E forms), 8.70-8.95 (1H, two broad peaks, Z/E forms). MW 4817.50. LCMS t_{R} (min): 1.55. MS (APCI+), m/z 482.05 [M+H]⁺. HPLC t_{R} (min): 10.61. M_{P} 205-206° C.

### 25. N*2*-(1-Methanesulfonyl-piperidin-4-yl)-N*4*-(2-methoxy-pyridin-4-yl)-6-(2,2,2-trifluoro-ethoxy)-1,3,5-triazine-2,4-diamine

¹H-NMR (400 MHz, DMSO-D₆) δ_{H}: 1.60 (2H, m), 1.98 (2H, m), 2.89 (5H, superposition of m and s), 3.60 (2H, broad peaks), 3.82 (3H, s), 3.95 (1H, broad peak), 4.99 (2H, superposition of two broad q, J=7.5 Hz, Z/E forms), 7.26 (1H, broad peak, Z/E forms), 7.30-7.36 (1H, two broad peaks, Z/E forms), 7.89-7.94 (1H, two broad peaks, Z/E forms), 7.96-8.00 (1H, broad d, J=7.5 Hz, Z/E forms), 9.82-9.98 (1H, broad d, Z/E forms). MW 477.5. LCMS t_{R} (min): 1.52. MS (APCI+), m/z 478.15 [M+H]⁺. HPLC t_{R} (min): 9.89. M_{P} 188-191° C.

### 26. N*2*-(1-Methanesulfonyl-piperidin-4-yl)-N*4*-(2-pyrrolidin-1-yl-pyridin-4-yl)-6-(2,2,2-trifluoro-ethoxy)-1,3,5-triazine-2,4-diamine

¹H-NMR (400 MHz, DMSO-D₆) δ_{H}: 1.71 (2H, m), 1.92 (6H, m), 2.82 (2H, m), 2.89 (3H, s), 3.36 (4H, broad peak, Z/E forms), 3.61 (2H, m), 4.01 (1H, broad peak, Z/E forms), 4.98 (2H, broad q, J=7.5 Hz), 6.74-6.84 (1H, two broad peaks, Z/E forms), 7.01-7.15 (1H, two broad peaks, Z/E forms), 7.71-7.91 (1H, two broad peaks, Z/E forms), 7.95 (1H, broad peak, Z/E forms), 9.59-9.78 (1H, two broad peaks, Z/E forms). MW 516.5. LCMS t_{R} (min): 1.54. MS (APCI+), m/z 517.22 [M+H]⁺. HPLC t_{R} (min): 10.48. M_{P} 146-148° C.

### 27. N*2*-(3-Dimethylamino-phenyl)-N*4*-(1-methanesulfonyl-piperidin-4-yl)-6-(2,2,2-trifluoro-ethoxy)-1,3,5-triazine-2,4-diamine

Yield 134 mg, 83%. ¹H-NMR (400 MHz, DMSO-D₆) δ_{H}: 1.60 (2H, m), 1.94 (2H, m), 2.90 (5H, superposition of s and m), 3.19-3.28 (6H, two s, Z/E forms), 3.57 (2H, m), 3.95 (1H, broad peak, Z/E forms), 4.95 (2H, q, J=7.5 Hz), 6.40 (1H, d, J=8.5 Hz), 6.97-7.07 (1H, two broad peaks, Z/E forms), 7.10 (1H, d, J=8.5 Hz), 7.17 (1H, s), 7.50-7.65 (1H, two broad peaks, Z/E forms), 9.20-9.38 (1H, two broad peaks, Z/E forms). MW 489.5. LCMS t_{R} (min): 1.63. MS (APCI+), m/z 490.14 [M+H]⁺. HPLC t_{R} (min): 10.31. M_{P} 176-178° C.

### 28. 5-[4-(1-Methanesulfonyl-piperidin-4-ylamino)-6-(2,2,2-trifluoro-ethoxy)-[1,3,5]-triazin-2-ylamino]-1,3-dihydro-benzoimidazol-2-one

Yield 195 mg, 66%. ¹H-NMR (400 MHz, DMSO-D₆) δ_{H}: 1.60 (2H, broad peak, Z/E forms), 1.98 (2H, broad peak, Z/E forms), 2.90 (1H, superposition of s and m), 3.60 (2H, m), 3.94 (1H, broad peak, Z/E forms), 4.92 (2H, broad q, J=7.5 Hz), 6.84 (1H, broad peak, Z/E forms), 7.15 (1H, broad peak, Z/E forms), 7.21-7.42 (1H, two broad peaks, Z/E forms), 7.42-7.58 (1H, two broad peaks, Z/E forms), 9.19-9.42 (1H, two broad peaks, Z/E forms), 10.33 (1H, broad peak, Z/E forms), 10.40-10.48 (1H, two broad peaks, Z/E forms). MW 502.47. LCMS t_{R} (min): 1.53. MS (APCI+), m/z 503.12 [M+H]⁺. HPLC t_{R} (min): 10.30. M_{P} 210-212° C.

### 29. [4-(3-Benzylamino-pyrrolidin-1-yl)-6-(2,2,2-trifluoro-ethoxy)-[1,3,5]triazin-2-yl]-(1-methanesulfonyl-piperidin-4-yl)-amine

¹H-NMR (400 MHz, DMSO-D₆) δ_{H}: 1.54 (2H, m), 1.78-1.90 (2H, two broad peaks, Z/E forms), 2.00 (2H, m), 2.82 (5H, superposition of m and s), 3.30 (2H, broad t, J=7.5 Hz), 3.45 (1H, broad peak, Z/E forms), 3.57 (5H, superposition of two m), 3.72 (2H, broad peak, Z/E forms), 3.90 (1H, broad peak, Z/E forms), 4.88 (2H, broad q, J=7.5 Hz), 7.21 (1H, broad t, J=8.5 Hz), 7.29 (2H, t, J=8.5 Hz), 7.34 (2H, broad peak, Z/E forms) 7.39 (2H, d, J=8.5 Hz). MW 529.58. LCMS t_{R} (min): 1.51. MS (APCI+), m/z 530.17 [M+H]⁺. HPLC t_{R} (min): 10.28. M_{P} 163-165° C.

### 30. N-(4-Fluoro-3-morpholin-4-ylmethyl-phenyl)-N′-(1-methanesulfonyl-piperidin-4-yl)-6-(2,2,2-trifluoro-ethoxy)-[1,3,5]triazine-2,4-diamine

To a suspension of {2-Fluoro-5-[4-(1-methanesulfonyl-piperidin-4-ylamino)-6-(2,2,2-trifluoro-ethoxy)-[1,3,5]triazin-2-ylamino]-phenyl}-morpholin-4-yl-methanone (146 mg, 0.25 mmol) in THF (4 mL) LiAlH₄ (29 mg, 0.76 mmol) was added portionwise at room temperature. The resulting mixture was stirred at room temperature for 3 hours, diluted with water and ethyl acetate. Then the mixture was filtered through “Celite”, washed with ethyl acetate and dichloromethane. The water layer was basified to pH˜10 by addition of K₂CO₃ and extracted with dichloromethane. The combined organic phases were dried over Na₂SO₄ and concentrated. Purification by column chromatography on silica gel (ethyl acetate) gave a final compound. Yield 82 mg, 58%. ¹H-NMR (400 MHz, DMSO-D₆) δ_{H}: 1.55 (2H, m), 1.88 (2H, m), 2.43 (2H, broad peak, Z/E forms), 2.80 (2H, m), 2.88 (3H, s), 3.43 (2H, s), 3.51 (2H, m), 3.60 (4H, broad peak, Z/E forms), 3.80-3.89 (1H, two broad peaks, Z/E forms), 4.90 (2H, broad q, J=7.5 Hz, Z/E forms), 7.13 (2H, superposition of two m, Z/E forms), 7.48 (1H, broad peak, Z/E forms), 7.63 (1H, broad peak, Z/E forms), 8.92-9.10 (1H, two broad peaks, Z/E forms). MW 563.58. LCMS t_{R} (min): 1.44. MS (APCI+), m/z 564.07 [M+H]⁺. HPLC t_{R} (min): 10.07. M_{P} 96-98° C.

### 31. 5-[4-(1-Methanesulfonyl-piperidin-4-ylamino)-6-(2,2,2-trifluoro-ethoxy)-[1,3,5]triazin-2-ylamino]-isoindole-1,3-dione

Yield 30 mg, 2% (for three steps). ¹H-NMR (400 MHz, DMSO-D₆) δ_{H}: 1.61 (2H, m), 1.91 (2H, m), 2.51 (2H, m), 2.91 (3H, s), 3.64 (2H, m), 3.91 (1H, broad peak, Z/E forms), 4.92 (2H, superposition of two q, J=7.5 Hz, Z/E forms), 7.71-7.72 (1H, two broad d, J=8.5 Hz, Z/E forms), 7.81 (1H, broad d, J=8.5 Hz), 7.95-8.21 (1H, two broad peaks, Z/E forms), 10.09-10.29 (1H, two broad peaks, Z/E forms), 11.05 (1H, broad peak, Z/E forms). MW 515.47. LCMS t_{R} (min): 1.65. MS (APCI+), m/z 516.04 [M+H]⁺. HPLC t_{R} (min): 12.26. M_{P} 311-312° C.

### 32. N-(3-Dimethylaminomethyl-4-fluoro-phenyl)-N′-(1-methanesulfonyl-piperidin-4-yl)-6-(2,2,2-trifluoro-ethoxy)-[1,3,5]triazine-2,4-diamine

Yield 85 mg, 58%. ¹H-NMR (400 MHz, DMSO-D₆) δ_{H}: 1.55 (2H, m), 1.90 (2H, m), 2.15 (6H, s), 2.80 (2H, m), 2.88 (3H, s), 3.35 (2H, s), 3.53 (2H, m), 3.88 (1H, broad peak, Z/E forms), 4.90 (2H, broad q, J=7.5 Hz, Z/E forms), 7.08 (1H, broad peak, Z/E forms), 7.14 (1H, m), 7.46 (1H, broad peak, Z/E forms), 7.63 (1H, broad peak, Z/E forms), 8.90-9.08 (1H, two broad peaks, Z/E forms). MW 521.54. LCMS t_{R} (min): 1.45. MS (APCI+), m/z 521.79 [M+H]⁺. HPLC t_{R} (min): 9.97. M_{P} 81-83° C.

The following compounds were synthesized in two approaches. One way is R4-R6-R2 route and the R4s were synthesized in two steps starting from acylation of acids (1) with amines and reduction of nitro compounds (2) with SnCl₂*2H₂O. The amide moieties were further reduced with LiAlH₄.on the last step to provide the desired compounds. Another approach is to reduced 2 with LiAlH₄.followed by reduction of nitrous to amines (5), which were introduced to triazine core in the first step.

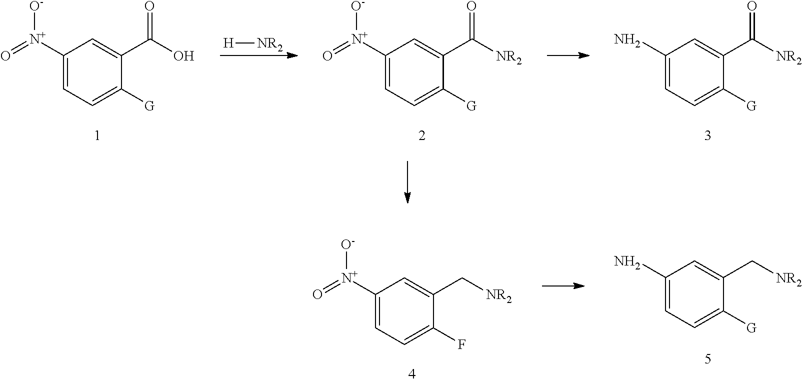

### 33. {2-Fluoro-5-[4-(1-methanesulfonyl-piperidin-4-ylamino)-6-(2,2,2-trifluoro-ethoxy)-[1,3,5]triazin-2-ylamino]-phenyl}-morpholin-4-yl-methanone

A mixture of 2-fluoro-5-nitro-benzoic acid (2.0 g, 10.80 mmol), morpholine (941 mg, 10.80 mmol), TBTU (3.712 g, 11.56 mmol), DIPEA (2.79 g, 21.61 mmol) in dichloromethane (100 mL) was stirred at room temperature for 18 hours, diluted with 5% aqueous solution of K₂CO₃ and 1N aqueous solution of HCl. The resulting mixture was extracted with dichloromethane. The combined organic phases were concentrated. Purification by column chromatography on silica gel (ethyl acetate) gave (2-Fluoro-5-nitro-phenyl)-morpholin-4-yl-methanone. Yield 1.518 g, 55%. MW 254.22. LCMS t_{R} (min): 1.41. MS (APCI+), m/z 255.10 [M+H]⁺.

A mixture of (2-Fluoro-5-nitro-phenyl)-morpholin-4-yl-methanone (1.518 g, 8.97 mmol), SnCl₂*2H₂O (6.737 g, 29.86 mmol) in ethanol (15 mL) was refluxed for 40 minutes, cooled down to room temperature and poured into ice. To the resulting mixture K₂CO₃ was added to reach pH=9. The formed precipitate was filtered through “Celite”, washed with ethyl acetate, acetone and dichloromethane. The combined organic phases were washed with brine, dried over Na₂SO₄, concentrated and dried giving (5-Amino-2-fluoro-phenyl)-morpholin-4-yl-methanone. Yield 1.416 g, 94%. MW 224.24. LCMS t_{R} (min): 1.28. MS (APCI+), m/z 225.14 [M+H]⁺.

To a solution of cyanuric chloride (1.416 g, 7.73 mmol) in THF (10 mL) a mixture of (5-Amino-2-fluoro-phenyl)-morpholin-4-yl-methanone (1.733 g, 7.73 mmol), DIPEA (1.10 g, 8.50 mmol) in THF (15 mL) was added. The resulting mixture was stirred at −20° C. for 3 hours, warmed up to room temperature and diluted with water. The obtained precipitate was filtered off, washed with water, dried on air giving [3-(4,6-Dichloro-[1,3,5]-triazin-2-ylamino)-2-fluoro-phenyl]-morpholin-4-yl-methanone. Yield 2.604 g, 91%. MW 372.19. LCMS t_{R} (min): 1.56. MS (APCI+), m/z 371.98, 373.96 [M+H]⁺.

To a solution of [3-(4,6-Dichloro-[1,3,5]triazin-2-ylamino)-2-fluoro-phenyl]-morpholin-4-yl-methanone (972 mg, 2.61 mmol) in acetonitrile (20 mL) a mixture of 1-methanesulfonyl-piperidin-4-ylamine hydrochloride (561 mg, 2.61 mmol), DIPEA (1.015 g, 7.83 mmol) in acetonitrile (20 mL) was added at 0° C. The resulting mixture was stirred at 0° C. for 1 hour, then warmed up to room temperature, stirred for 50 hours and concentrated. The formed residue was washed with water, refluxed in ethyl acetate for 1 hour, filtered off and dried giving {5-[4-Chloro-6-(1-methanesulfonyl-piperidin-4-ylamino)-[1,3,5]-triazin-2-ylamino]-2-fluoro-phenyl}-morpholin-4-yl-methanone. Yield 1.004 g, 75%. MW 513.98. LCMS t_{R} (min): 1.56. MS (APCI+), m/z 514.03, 516.07 [M+H]⁺.

A mixture of Chloro-6-(1-methanesulfonyl-piperidin-4-ylamino)-[1,3,5]triazin-2-ylamino]-2-fluoro-phenyl}-morpholin-4-yl-methanone (400 mg, 0.78 mmol), 2,2,2-trifluoroethanol (234 mg, 2.33 mmol), K₂CO₃ (323 mg, 2.33 mmol) in DMSO (4 mL) was stirred at 100° C. for 6 hours. The resulting mixture was cooled down to room temperature and diluted with water. The formed precipitate was filtered off, washed with ethyl acetate. Purification by column chromatography on silica gel (dichloromethane/acetone, 4/1) gave {2-Fluoro-5-[4-(1-methanesulfonyl-piperidin-4-ylamino)-6-(2,2,2-trifluoro-ethoxy)-[1,3,5]triazin-2-ylamino]-phenyl}-morpholin-4-yl-methanone. Yield 340 mg, 76%. ¹H-NMR (400 MHz, DMSO-D₆) δ_{H}: 1.61 (2H, m), 1.91 (2H, m), 2.91 (5H, superposition of s (3H) and broad peak (2H)), 3.51 (6H, m), 3.62 (4H, broad peak, Z/E forms), 3.85 (1H, broad peak, Z/E forms), 4.91 (2H, q, J=7.5 Hz), 7.32 (1H, d/d, J=8.5/8.5 Hz), 7.69-7.52 (1H, two broad peaks, Z/E forms), 7.69-8.12 (1H, two broad peaks, Z/E forms), 9.12 (1H, broad peak, Z/E forms). MW 577.56. LCMS t_{R} (min): 1.66. MS (APCI+), m/z 578.02, 579.16 [M+H]⁺. HPLC t_{R} (min): 12.32. M_{P} 217-219° C.

### 34. 2-Fluoro-5-[4-(1-methanesulfonyl-piperidin-4-ylamino)-6-(2,2,2-trifluoro-ethoxy)-[1,3,5]triazin-2-ylamino]-N,N-dimethyl-benzamide

Yield 326 mg, 72%. ¹H-NMR (400 MHz, DMSO-D₆) δ_{H}: 1.61 (2H, m), 1.91 (2H, m), 2.81 (5H, superposition of s (3H) and m (2H)), 2.98 (6H, s), 3.51 (2H, m), 3.82-3.92 (1H, two broad peaks, Z/E forms), 4.91 (2H, q, J=7.5 Hz), 7.18-7.29 (1H, two broad peaks, Z/E forms), 7.29 (1H, d/d, J=8.0/8.5 Hz), 7.58-7.65 (1H, two broad peaks, Z/E forms), 7.72-7.98 (1H, two broad peaks, Z/E forms), 9.09-9.31 (1H, two broad peaks, Z/E forms). MW 535.52. LCMS t_{R} (min): 1.68. MS (APCI+), m/z 536.08 [M+H]⁺. HPLC t_{R} (min): 12.57. Mp 192-193° C.

### 35. {{2-Fluoro-5-[4-(1-methanesulfonyl-piperidin-4-ylamino)-6-(2,2,2-trifluoro-ethoxy)-[1,3,5]triazin-2-ylamino]-phenyl}-pyrrolidin-1-yl-methanone

Yield 283 mg, 28%. ¹H-NMR (400 MHz, DMSO-D₆) δ_{H}: 1.53 (2H, m), 1.83 (6H, m), 2.80 (2H, m), 2.88 (3H, s), 3.42 (4H, broad peak, Z/E forms), 3.52 (2H, m), 3.88 (1H, broad peak, Z/E forms), 4.92 (2H, q, J=7.5 Hz), 7.30 (2H, superposition of two m, Z/E forms), 7.55-7.73 (1H, two broad peaks, Z/E forms), 7.73-8.02 (1H, two broad peaks, Z/E forms), 9.10-9.30 (1H, two broad peaks, Z/E forms). MW 561.56. LCMS t_{R} (min): 1.74. MS (APCI+), m/z 562.01 [M+H]⁺. HPLC t_{R} (min): 13.40. M_{P} 110-112° C.

### Preparation of O,N,N-Triazines in Table 16

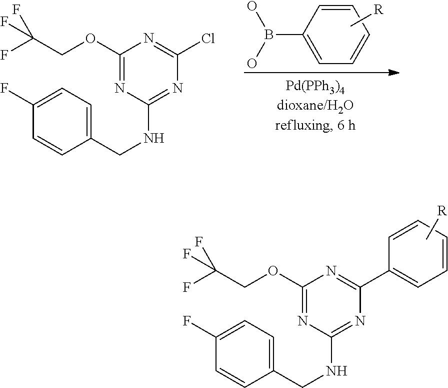

O,N,N-Triazine were synthesized by Suzuki coupling of the chloro-intermediate I-36 with aryl boronic acid.

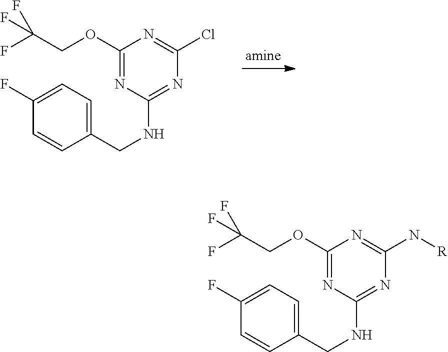

O,N,N-Triazine were synthesized by several methods:

Method A: A mixture of compound I-36 (337 mg, 1.0 mmol), corresponding aniline (1.0 mmol), K₂CO₃ (400 mg, 3 mmol) and DMSO (5.0 mL) was stirred at 80-150° C. for 1-4 hours (TLC control), cooled down to room temperature, diluted with water (50 mL). The formed solid was collected by filtration or the reaction mixture was extracted with dichloromethane. Purification by column chromatography on silica gel or by other appropriate methods furnished final compound.

Method B: A mixture of compound I-36 (397 mg, 1.18 mmol), corresponding aniline (1.18 mmol), DIPEA (460 mg, 3.54 mmol) and acetonitrile (6 mL) was stirred at room temperature for 4-60 hours, diluted with water. The formed solid was collected by filtration. Purification by appropriate method gave compound.

Method C: To a solution I-36 (337 mg, 1 mmol) in AcOH (3 mL) sodium acetate (100 mg, 1.22 mmol) and corresponding amine (1.15 mmol) were added. The mixture was stirred at 50° C.-90° C. for 3 hours, cooled down to room temperature, neutralized with aqueous ammonia solution and extracted with ethyl acetate. The organic layers was combined, dried over Na₂SO₄ and concentrated. The residue was purified by appropriate method giving final compound.

Method D: A mixture of compound I-36 (397 mg, 1.18 mmol), corresponding amine (1.30 mmol), NEt₃ (360 mg, 3.54 mmol) or DIPEA (457 mg, 3.54 mmol) and acetonitrile (6 mL) was stirred at refluxing for 2-12 hours (TLC control), cooled to room temperature, diluted with water and extracted with chloroform. The combined organic phases were concentrated. Purification by column chromatography gave final compound.

Method E: The mixture of compound I-36 (317 mg, 0.94 mmol), corresponding amine (1.13 mmol), NaHCO₃ (95 mg, 1.13 mmol) or K₂CO₃ (156 mg, 1.13 mmol) and acetonitrile (3 mL) was stirred at 60° C. or at refluxing for 2-24 hours. The mixture was diluted with water (20 mL), extracted with chloroform (2×30 mL), dried over Na₂SO₄ and concentrated. The residue was purified by column chromatography and preparative TLC giving final compounds.

TABLE 16

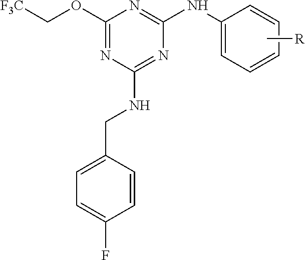

| En | Structure | IUPAC Name | MW | Formula |
|---|---|---|---|---|
| 1 | 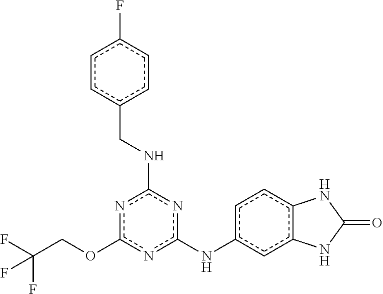 | 5-({4-[(4-fluorobenzyl) amino]-6-(2,2,2- trifluoroethoxy)-1,3,5- triazin-2-yl}amino)-1,3- dihydro-2H-benzimidazol- 2-one | 449.4 | C19H15F4N7O2 |
| 2 | 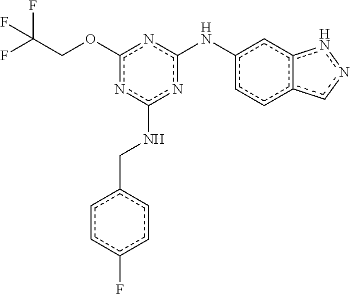 | N-(4-fluorobenzyl)-N′-(1H- indazol-6-yl)-6-(2,2,2- trifluoroethoxy)-1,3,5- triazine-2,4-diamine | 433.4 | C19H15F4N7O |
| 3 | 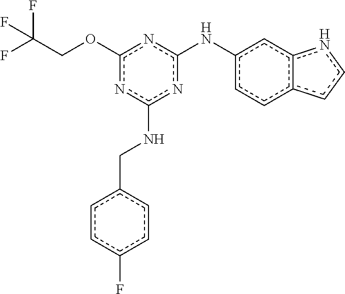 | N-(4-fluorobenzyl)-N′-(1H- indol-6-yl)-6-(2,2,2- trifluoroethoxy)- 1,3,5-triazine-2,4-diamine | 432.4 | C20H16F4N6O |
| 4 | 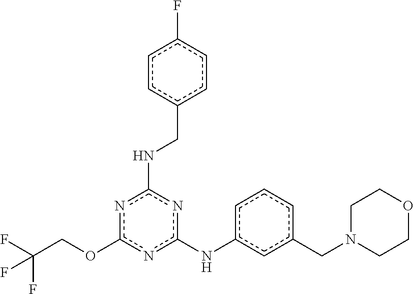 | N-(4-fluorobenzyl)-N′-[3- (morpholin-4-ylmethyl) phenyl]-6-(2,2,2- trifluoroethoxy)-1,3,5- triazine-2,4-diamine | 492.5 | C23H24F4N6O2 |
| 5 | 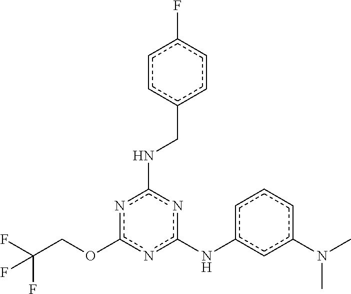 | N-[3-(dimethylamino) phenyl]-N′-(4-fluorobenzyl)- 6-(2,2,2-trifluoroethoxy)- 1,3,5-triazine-2,4-diamine | 436.4 | C20H20F4N6O |
| 6 | 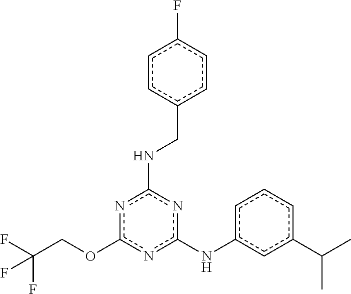 | N-(4-fluorobenzyl)-N′-[3- (propan-2-yl)phenyl]-6- (2,2,2-trifluoroethoxy)- 1,3,5-triazine-2,4-diamine | 435.4 | C21H21F4N5O |
| 7 | 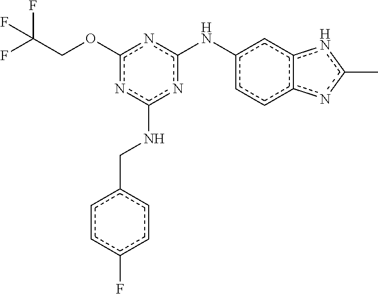 | N-(4-fluorobenzyl)-N′-(2- methyl-1H-benzimidazol-6- yl)-6-(2,2,2-trifluoroethoxy)- 1,3,5-triazine-2,4-diamine | 447.4 | C20H17F4N7O |
| 8 | 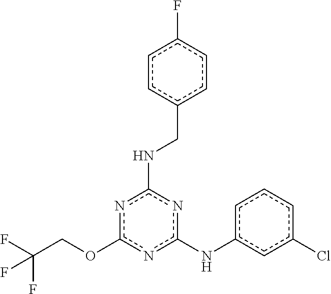 | N-(3-chlorophenyl)-N′-(4- fluorobenzyl)-6-(2,2,2- trifluoroethoxy)-1,3,5- triazine-2,4-diamine | 427.8 | C18H14CIF4N5O |
| 9 | 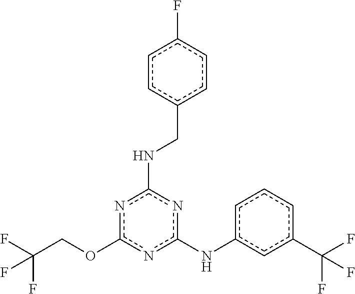 | N-(4-fluorobenzyl)-6-(2,2,2- trifluoro-ethoxy)-N′-[3- (trifluoromethyl)phenyl]- 1,3,5-triazine-2,4-diamine | 461.3 | C19H14F7N5O |
| 10 | 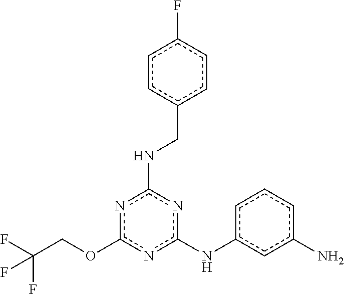 | N-(3-aminophenyl)-N′-(4- fluorobenzyl)-6-(2,2,2- trifluoroethoxy)-1,3,5- triazine-2,4-diamine | 408.4 | C18H16F4N6O |
| 11 | 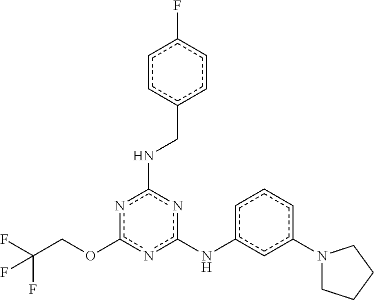 | N-(4-fluorobenzyl)-N′-[3- (pyrrolidin-1-yl)phenyl]-6- (2,2,2-trifluoroethoxy)- 1,3,5-triazine-2,4-diamine | 462.4 | C22H22F4N6O |
| 12 | 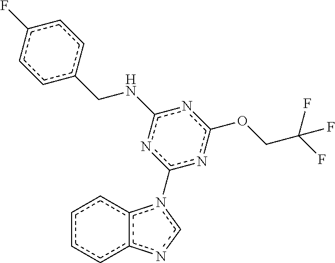 | 4-(1H-benzimidazol-1-yl)- N-(4-fluorobenzyl)-6- (2,2,2-trifluoroethoxy)- 1,3,5-triazin-2-amine | 418.3 | C19H14F4N6O |
| 13 | 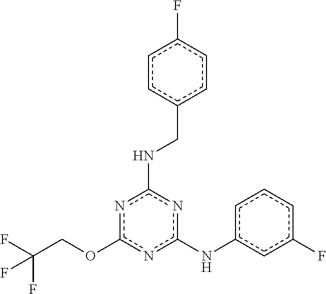 | N-(4-fluorobenzyl)-N′-(3- fluorophenyl)-6-(2,2,2- trifluoroethoxy)-1,3,5- triazine-2,4-diamine | 411.3 | C18H14F5N5O |
| 14 | 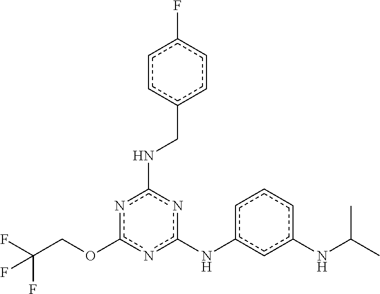 | N-(4-fluorobenzyl)-N′-[3- (propan-2-ylamino)phenyl]- 6-(2,2,2-trifluoroethoxy)- 1,3,5-triazine-2,4-diamine | 450.4 | C21H22F4N6O |
| 15 | 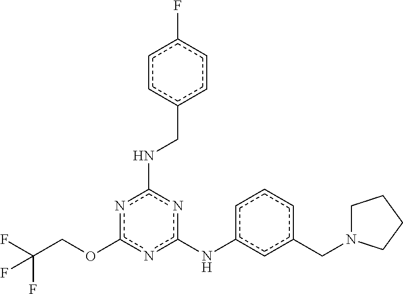 | N-(4-fluorobenzyl)-N′-[3- (pyrrolidin-1-ylmethyl) phenyl]-6-(2,2,2- trifluoroethoxy)-1,3,5- triazine-2,4-diamine | 476.5 | C23H24F4N6O |
| 16 | 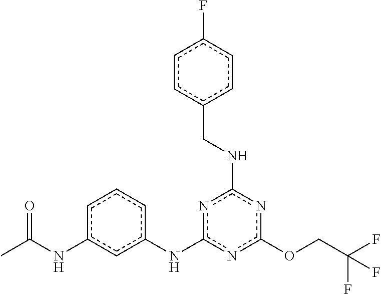 | N-[3-({4-[(4- fluorobenzyl)amino]-6- (2,2,2-trifluoroethoxy)- 1,3,5-triazin-2-yl} amino)phenyl]acetamide | 450.4 | C20H18F4N6O2 |
| 17 | 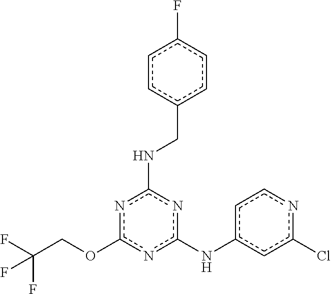 | N-(2-chloropyridin-4-yl)- N′-(4-fluorobenzyl)-6- (2,2,2-trifluoroethoxy)- 1,3,5-triazine-2,4-diamine | 428.8 | C17H13ClF4N6O |
| 18 | 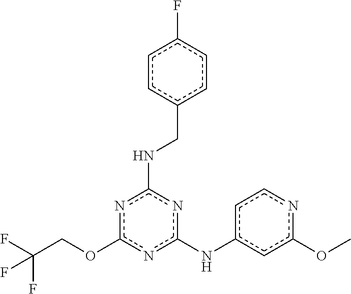 | N-(4-fluorobenzyl)-N′-(2- methoxypyridin-4-yl)-6- (2,2,2-trifluoroethoxy)- 1,3,5-triazine-2,4-diamine | 424.4 | C18H16F4N6O2 |
| 19 | 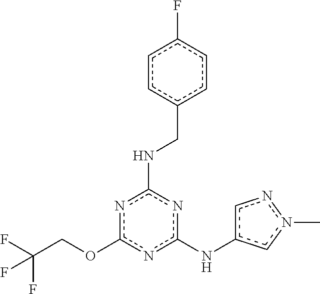 | N-(4-fluorobenzyl)-N′-(1- methyl-1H-pyrazol-4-yl)-6- (2,2,2-trifluoroethoxy)- 1,3,5-triazine-2,4-diamine | 397.3 | C16H15F4N7O |
| 20 | 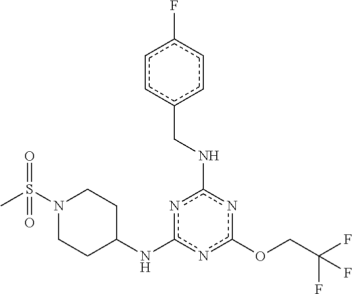 | N-(4-fluorobenzyl)-N′-[1- (methylsulfonyl)piperidin- 4-yl]-6-(2,2,2- trifluoroethoxy)-1,3,5- triazine-2,4-diamine | 478.5 | C18H22F4N6O3S |
| 21 | 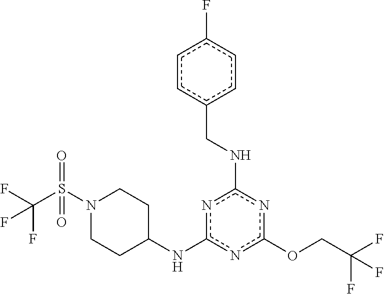 | N-(4-fluorobenzyl)-6-(2,2,2- trifluoroethoxy)-N′-{1- [(trifluoromethyl)sulfonyl] piperidin-4-yl}-1,3,5- triazine-2,4-diamine | 532.4 | C18H19F7N6O3S |
| 22 | 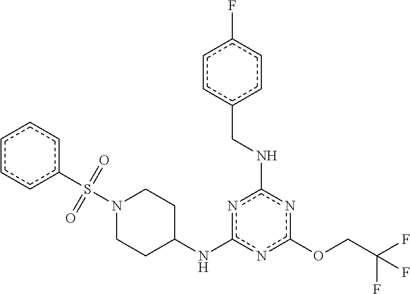 | N-(4-fluorobenzyl)-N′-[1- (phenylsulfonyl)piperidin-4- yl]-6-(2,2,2-trifluoroethoxy)- 1,3,5-triazine-2,4-diamine | 540.5 | C23H24F4N6O3S |
| 23 | 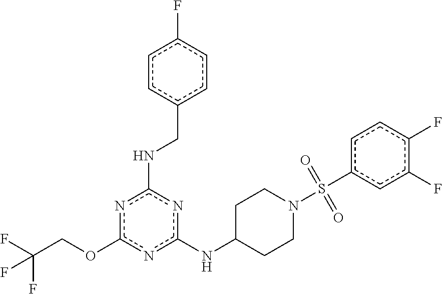 | N-{1-[(3,4- difluorophenyl)sulfonyl] piperidin-4-yl}-N′-(4- fluorobenzyl)-6-(2,2,2- trifluoroethoxy)-1,3,5- triazine-2,4-diamine | 576.5 | C23H22F6N6O3S |
| 24 | 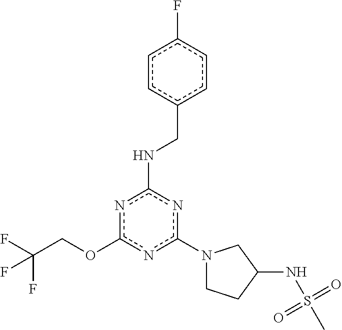 | N-(1-{4-[(4-fluorobenzyl) amino]-6-(2,2,2-trifluoro- ethoxy)-1,3,5-triazin-2-yl} pyrrolidin-3-yl) methanesulfonamide | 464.4 | C17H20F4N6O3S |
| 25 | 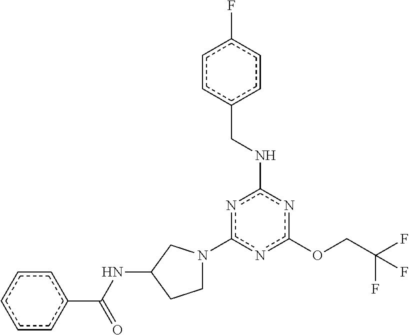 | N-(1-{4-[(4-fluorobenzyl) amino]-6-(2,2,2-trifluoro- ethoxy)-1,3,5-triazin-2-yl} pyrrolidin-3-yl)benzamide | 490.5 | C23H22F4N6O2 |
| 26 | 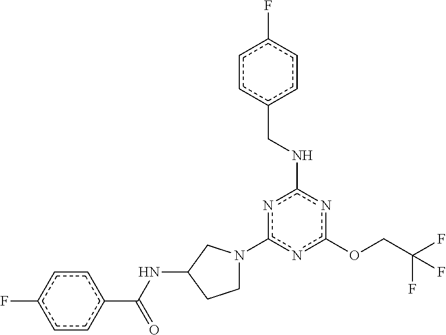 | 4-fluoro-N-(1-{4-[(4- fluorobenzyl)amino]-6- (2,2,2-trifluoroethoxy)- 1,3,5-triazin-2-yl} pyrrolidin-3-yl)benzamide | 508.4 | C23H21F5N6O2 |
| 27 | 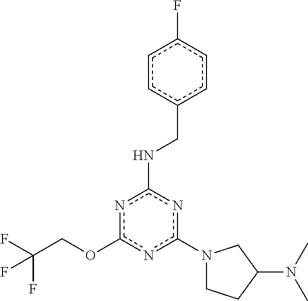 | 4-[3-(dimethylamino) pyrrolidin-1-yl]-N-(4- fluorobenzyl)-6- (2,2,2-trifluoroethoxy)- 1,3,5-triazin-2-amine | 414.4 | C18H22F4N6O |
| 28 | 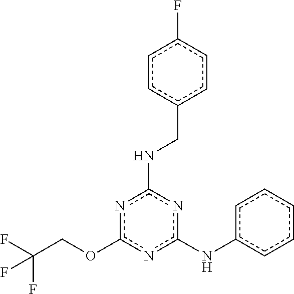 | N-(4-fluorobenzyl)-N′- phenyl-6-(2,2,2- trifluoroethoxy)-1,3,5- triazine-2,4-diamine | 393.3 | C18H15F4N5O |
| 29 | 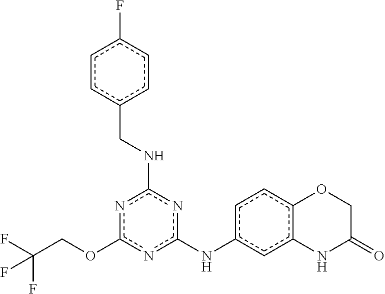 | 6-({4-[(4-fluorobenzyl) amino]-6-(2,2,2- trifluoroethoxy)-1,3,5- triazin-2-yl}amino)-2H- 1,4-benzoxazin-3(4H)-one | 464.4 | C20H16F4N6O3 |
| 30 | 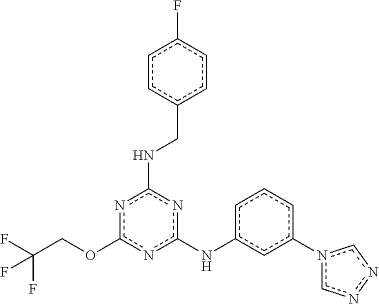 | N-(4-fluorobenzyl)-N′-[3- (4H-1,2,4-triazol-4-yl) phenyl]-6-(2,2,2- trifluoroethoxy)-1,3,5- triazine-2,4-diamine | 460.4 | C20H16F4N8O |
| 31 | 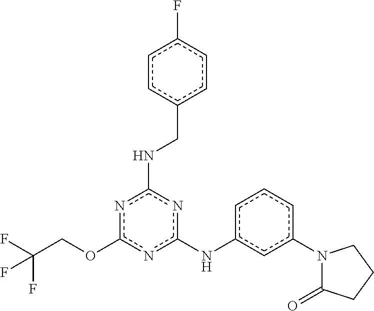 | 1-[3-({4-[(4-fluorobenzyl) amino]-6-(2,2,2-trifluoro- ethoxy)-1,3,5-triazin-2- yl}amino)phenyl] pyrrolidin-2-one | 476.4 | C22H20F4N6O2 |
| 32 | 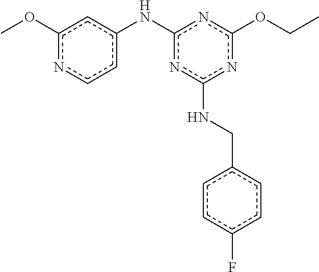 | 6-ethoxy-N-(4- fluorobenzyl)-N′-(2- methoxypyridin-4-yl)- 1,3,5-triazine-2,4- diamine | 370.4 | C18H19FN6O2 |
| 33 | 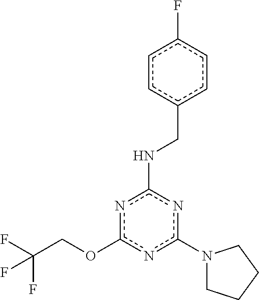 | N-(4-fluorobenzyl)-4- (pyrrolidin-1-yl)-6-(2,2,2- trifluoroethoxy)- 1,3,5-triazin-2-amine | 371.3 | C16H17F4N5O |
| 34 | 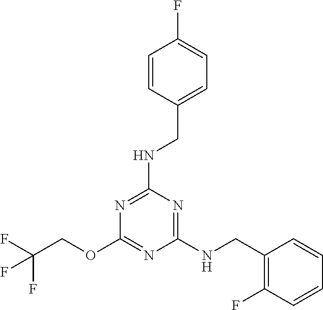 | N-(2-fluorobenzyl)-N′-(4- fluorobenzyl)-6-(2,2,2- trifluoroethoxy)-1,3,5- triazine-2,4-diamine | 425.3564 | C19H16F5N5O |
| 35 | 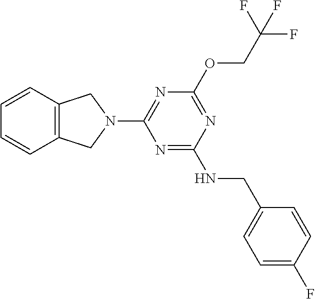 | 4-(1,3-dihydro-2H-isoindol- 2-yl)-N-(4-fluorobenzyl)-6- (2,2,2-trifluoroethoxy)- 1,3,5-triazin-2-amine | 419.3767 | C20H17F4N5O |
| 36 | 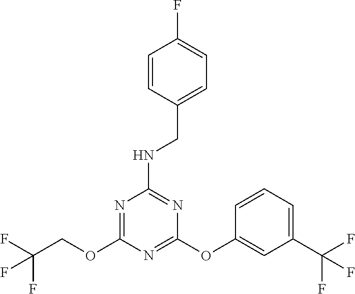 | N-(4-fluorobenzyl)-4-(2,2,2- trifluoroethoxy)-6-[3- (trifluoromethyl)phenoxy]- 1,3,5-triazin-2-amine | 462.3 | C19H13F7N4O2 |
| 37 | 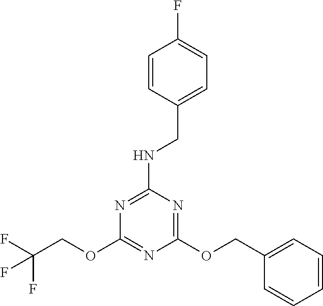 | 4-(benzyloxy)-N-(4- fluorobenzyl)-6-(2,2,2- trifluoroethoxy)-1,3,5- triazin-2-amine | 408.4 | C19H16F4N4O2 |
| 38 | 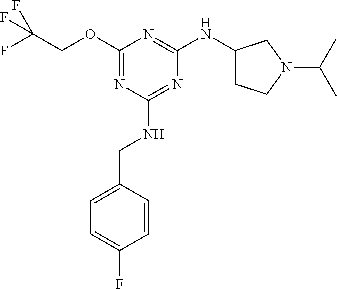 | N-(4-Fluoro-benzyl)-N′-(1- isopropyl-pyrrolidin-3-yl)-6- (2,2,2-trifluoro-ethoxy)- [1,3,5]triazine-2,4-diamine | 428.2 | C19H24F4N6O |

### Procedures and Analytical Data for Compounds in Table 16.

### 1. 5-[4-(4-Fluoro-benzylamino)-6-(2,2,2-trifluoro-ethoxy)-[1,3,5]triazin-2-ylamino]-1,3-dihydro-benzoimidazol-2-one

¹H-NMR (400 MHz, DMSO-D₆) δ_{H}: 4.50 (2H, broad), 4.90 (2H, superposition of two broad q, J=7.5 Hz, Z/E forms), 6.80 (1H, broad), 7.10 (2H, broad), 7.10-7.22 (1H, two broad signals, Z/E forms), 7.34 (2H, broad), 7.34-7.39 (1H, two broad signals, Z/E forms), 7.98-8.04 (1H, two broad signals, Z/E forms), 9.29-9.43 (1H, two broad signals, Z/E forms), 10.32 (1H, broad), 10.42-10.49 (1H, two broad signals, Z/E forms). MW 449.37. LCMS t_{R} (min): 1.68. MS (APCI), m/z 450.18 [M+H]⁺. HPLC t_{R} (min): 12.00. M_{P} 142-144° C.

### 2. N-(4-Fluoro-benzyl)-N′-(1H-indazol-6-yl)-6-(2,2,2-trifluoro-ethoxy)-[1,3,5]triazine-2,4-diamine

¹H-NMR (400 MHz, DMSO-D₆) δ_{H}: 4.45-4.63 (2H, broad doublets, J=7.5 Hz, Z/E forms), 4.95 (2H, superposition of two q, J=7.5 Hz), 7.12 (2H, broad), 7.28 (1H, d, J=8.5 Hz), 7.38 (2H, broad), 7.63 (1H, d, J=8.5 Hz), 7.94 (1H, s), 7.95-8.10 (1H, two s, Z/E forms), 8.08-8.23 (1H, broad, Z/E forms), 9.60-9.78 (1H, broad, Z/E forms), 12.70-12.86 (1H, broad, Z/E forms). MW 433.37. LCMS t_{R} (min): 1.81. MS (APCI), m/z 434.17 [M+H]⁺. HPLC t_{R} (min): 13.73. M_{P} 212-214° C.

### 3. N-(4-Fluoro-benzyl)-N′-(1H-indol-6-yl)-6-(2,2,2-trifluoro-ethoxy)-[1,3,5]triazine-2,4-diamine

¹H-NMR (400 MHz, DMSO-D₆) δ_{H}: 4.41-4.59 (2H, broad, Z/E forms), 4.92 (2H, superposition of two q, J=7.5 Hz, Z/E forms), 6.32 (1H, s), 7.09 (2H, m), 7.12-7.19 (1H, broad, Z/E forms), 7.22 (1H, broad), 7.36 (3H, broad), 7.72-7.87 (1H, broad, Z/E forms), 8.02 (1H, broad), 9.30-9.50 (1H, broad, Z/E forms), 10.91 (1H, broad). MW 432.39. LCMS t_{R} (min): 1.94. MS (APCI), m/z 433.07 [M+H]⁺. HPLC t_{R} (min): 15.02. M_{P} 155-157° C.

### 4. N-(4-Fluoro-benzyl)-N % (3-morpholin-4-ylmethyl-phenyl)-6-(2,2,2-trifluoro-ethoxy)-[1,3,5]triazine-2,4-diamine

¹H-NMR (400 MHz, DMSO-D₆) δ_{H}: 2.22-2.40 (4H, broad, Z/E forms), 3.30-3.45 (2H, two s, Z/E forms), 3.45-3.62 (4H, broad, Z/E forms), 4.45-4.60 (2H, broad, Z/E forms), 4.92 (2H, superposition of two q, J=7.5 Hz, Z/E forms), 6.92 (1H, t, J=8.5 Hz), 7.11 (2H, two d, J=8.5 Hz, Z/E forms), 7.19 (1H, d, J=8.5 Hz), 7.35 (2H, broad), 7.39-7.58 (1H, broad, Z/E forms), 7.61-7.74 (1H, broad, Z/E forms), 8.00-8.21 (1H, broad, Z/E forms), 9.41-9.62 (1H, broad, Z/E forms). MW 492.48. LCMS t_{R} (min): 1.61. MS (APCI), m/z 493.28 [M+H]⁺. HPLC t_{R} (min): 11.29. M_{P} 52-54° C.

### 5. N-(3-Dimethylamino-phenyl)-N′-(4-fluoro-benzyl)-6-(2,2,2-trifluoro-ethoxy)-[1,3,5]triazine-2,4-diamine

¹H-NMR (400 MHz, DMSO-D₆) δ_{H}: 2.75-2.90 (6H, two s, Z/E forms), 4.41-4.58 (2H, two broad doublets, J=7.5 Hz, Z/E forms), 4.94 (2H, superposition of two broad q, J=7.5 Hz, Z/E forms), 6.39 (1H, broad), 6.89-7.07 (1H, broad doublets, J=8.5 Hz, Z/E forms), 7.13 (4H, m), 7.34 (2H, broad), 8.08 (1H, broad), 9.22-9.42 (1H, broad, Z/E forms). MW 436.42. LCMS t_{R} (min): 1.87. MS (APCI), m/z 437.22 [M+H]⁺. HPLC t_{R} (min): 12.05. M_{P} 155-1570 g.

### 6. N-(4-Fluoro-benzyl)-N′-(3-isopropyl-phenyl)-6-(2,2,2-trifluoro-ethoxy)-[1,3,5]triazine-2,4-diamine

¹H-NMR (400 MHz, DMSO-D₆) δ_{H}: 1.12-1.20 (6H, two broad d, J=7.5 Hz, Z/E forms), 2.75-2.85 (1H, two broad signals, Z/E forms), 4.49-4.51 (2H, two broad signals, Z/E forms), 4.93 (2H, broad q, J=7.5 Hz, Z/E forms), 6.87 (1H, broad), 7.15 (3H, broad), 7.34 (3H, broad), 7.52-7.60 (1H, broad, Z/E forms), 8.07-8.13 (1H, two broad signals, Z/E forms), 9.40-9.55 (1H, two broad signals, Z/E forms). MW 435.43. LCMS t_{R} (min): 2.19. MS (APCI), m/z 436.17 [M+H]⁺. HPLC t_{R} (min): 17.44. M_{P} 140-142° C.

### 7. N-(4-Fluoro-benzyl)-N′-(2-methyl-3H-benzoimidazol-5-yl)-6-(2,2,2-trifluoro-ethoxy)-[1,3,5]triazine-2,4-diamine

Yield 105 mg, 30%. ¹H-NMR (400 MHz, DMSO-D₆) δ_{H}: 2.43 (3H, s), 4.50 (2H, broad), 4.95 (2H, broad q, J=7.5 Hz), 7.11 (2H, broad), 7.33 (1H, d, J=8.5 Hz), 7.36 (3H, broad), 7.80-7.85 (1H, broad, Z/E forms), 8.05 (1H, broad), 9.38-9.50 (1H, broad, Z/E forms), 12.00 (1H, broad). MW 447.40. LCMS t_{R} (min): 1.56. MS (APCI), m/z 448.13 [M+H]⁺. HPLC t_{R} (min): 10.91. M_{P} 216-218° C.

### 8. N-(3-Chloro-phenyl)-N′-(4-fluoro-benzyl)-6-(2,2,2-trifluoro-ethoxy)-[1,3,5]triazine-2,4-diamine

Method J. Yield 174 mg, 55%. ¹H-NMR (400 MHz, DMSO-D₆) δ_{H}: 4.50 (2H, broad), 4.95 (2H, broad q, J=7.5 Hz), 7.00 (1H, d, J=8.5 Hz), 7.12 (2H, d/d, J=8.0/8.5 Hz), 7.30 (1H, superposition of two t, J=8.5 Hz, Z/E forms), 7.39 (2H, broad m), 7.50-7.59 (1H, two d, J=8.5 Hz, Z/E forms), 7.85-7.90 (1H, broad, Z/E forms), 8.30 (1H, broad), 9.70-9.80 (1H, broad, Z/E forms). MW 427.79. LCMS t_{R} (min): 2.09. MS (APCI), m/z 428.13 [M+H]⁺. HPLC t_{R} (min): 16.68. M_{P} 148-150° C.

### 9. N-(4-Fluoro-benzyl)-N′-(3-fluoro-phenyl)-6-(2,2,2-trifluoro-ethoxy)-[1,3,5]triazine-2,4-diamine

Yield 50 mg, 10%. ¹H-NMR (400 MHz, DMSO-D₆) δ_{H}: 4.45-4.51 (2H, two d, J=7.5 Hz, Z/E forms), 4.98 (2H, broad q, J=7.5 Hz, Z/E forms), 7.12 (2H, broad m), 7.33 (3H, broad m), 7.48 (1H, m), 7.81-8.03 (1H, two broad d, J=8.5 Hz, Z/E forms), 8.10-8.20 (1H, broad, Z/E forms), 8.21-8.30 (1H, broad, Z/E forms), 9.83-9.96 (1H, broad, Z/E forms). MW 411.34. LCMS t_{R} (min): 2.01. MS (APCI), m/z 412.03 [M+H]⁺. HPLC t_{R} (min): 16.94. M_{P} 63-65° C.

### 10. N-(3-Amino-phenyl)-N′-(4-fluoro-benzyl)-6-(2,2,2-trifluoro-ethoxy)-[1,3,5]triazine-2,4-diamine

¹H-NMR (400 MHz, DMSO-D₆) δ_{H}: 4.50 (2H, broad, Z/E forms), 4.82 (2H, broad), 4.90 (2H, broad q, J=7.5 Hz, Z/E forms), 6.21 (1H, broad d, J=8.5 Hz, Z/E forms), 6.78-6.85 (1H, two broad signals, Z/E forms), 6.87 (2H, broad), 7.10 (2H, m, J=8.5 Hz), 7.35 (2H, broad), 7.95-8.05 (1H, two broad signals, Z/E forms), 9.18-9.30 (1H, two broad signals, Z/E forms). MW 408.36. LCMS t_{R} (min): 1.70. MS (APCI), m/z 409.08 [M+H]⁺. HPLC t_{R} (min): 11.15. M_{P} 171-173° C.

### 11. N-(4-Fluoro-benzyl)-N′-(3-pyrrolidin-1-yl-phenyl)-6-(2,2,2-trifluoro-ethoxy)-[1,3,5]triazine-2,4-diamine

¹H-NMR (400 MHz, DMSO-D₆) δ_{H}: 1.85 (2H, m), 1.95 (2H, m), 3.05 (2H, m), 3.20 (2H, m), 4.45-4.55 (2H, broad, Z/E forms), 4.95 (2H, superposition of two q, J=7.5 Hz, Z/E forms), 6.20 (1H, broad), 6.79-6.93 (1H, broad, Z/E forms), 6.99 (1H, t, J=7.5 Hz), 7.05 (1H, broad), 7.11 (2H, broad), 7.32 (2H, broad), 8.01-8.12 (1H, broad, Z/E forms), 9.25-9.40 (1H, broad, Z/E forms). MW 462.45. LCMS t_{R} (min): 2.15. MS (APCI), m/z 463.20 [M+H]⁺. HPLC t_{R} (min): 15.84. M_{P} 202-204° C.

### 12. [4-Benzoimidazol-1-yl-6-(2,2,2-trifluoro-ethoxy)-[1,3,5]triazin-2-yl]-(4-fluoro-benzyl)-amine

¹H-NMR (400 MHz, DMSO-D₆) δ_{H}: 4.61-4.70 (2H, two broad d, J=7.5 Hz, Z/E forms), 5.19 (2H, superposition of two broad q, J=7.5 Hz, Z/E forms), 7.17 (2H, broad, Z/E forms), 7.36 (1H, broad, Z/E forms), 7.45 (3H, broad, Z/E forms), 7.76 (1H, broad, Z/E forms), 8.29-8.63 (1H, two broad signals, Z/E forms), 9.02-9.09 (1H, two broad signals, Z/E forms), 9.14 (1H, broad). MW 418.36. LCMS t_{R} (min): 1.93. MS (APCI), m/z 419.21 [M+H]⁺. HPLC t_{R} (min): 15.41. M_{P} 215-21° C.

### 13. N-(4-Fluoro-benzyl)-6-(2,2,2-trifluoro-ethoxy)-N′-(3-trifluoromethyl-phenyl)-[1,3,5]triazine-2,4-diamine

Yield 50 mg, 15%. ¹H-NMR (400 MHz, DMSO-D₆) δ_{H}: 4.50 (2H, broad), 4.95 (2H, q, J=7.5 Hz), 6.79 (1H, t, J=8.5 Hz), 7.15 (2H, m), 7.25 (1H, superposition of two d, J=8.5 Hz, Z/E forms), 7.35 (2H, broad, Z/E forms), 7.35-7.45 (1H, broad, Z/E forms), 7.61-7.82 (1H, two d, J=8.5 Hz, Z/E forms), 8.27 (1H, broad), 9.70-9.80 (1H, broad, Z/E forms). MW 461.35. LCMS t_{R} (min): 2.10. MS (APCI), m/z 462.12 [M+H]⁺. HPLC t_{R} (min): 16.09. M_{P} 47-49° C.

### 14. N-(4-Fluoro-benzyl)-N′-(3-isopropylamino-phenyl)-6-(2,2,2-trifluoro-ethoxy)-[1,3,5]triazine-2,4-diamine

¹H-NMR (400 MHz, DMSO-D₆) δ_{H}: 1.05-1.12 (6H, broad d, J=7.5 Hz, Z/E forms), 3.40-3.50 (1H, two broad signals, Z/E forms), 4.49-4.55 (2H, two broad d, J=7.5 Hz, Z/E forms), 4.95 (2H, broad q, J=7.5 Hz), 5.10 (1H, broad), 6.25 (1H, broad d, J=8.5 Hz), 6.75-6.88 (1H, two broad d, J=8.5 Hz, Z/E forms), 6.92 (2H, broad), 7.16 (2H, m, J=8.5 Hz), 7.35 (2H, broad), 7.98-8.08 (1H, two broad signals, Z/E forms), 9.22-9.33 (1H, two broad signals, Z/E forms). MW 450.44. LCMS t_{R} (min): 1.77. MS (APCI), m/z 451.16 [M+H]⁺. HPLC t_{R} (min): 11.78. M_{P} 144-146° C.

### 15. N-(4-Fluoro-benzyl)-N′-(3-pyrrolidin-1-ylmethyl-phenyl)-6-(2,2,2-trifluoro-ethoxy)-[1,3,5]triazine-2,4-diamine

¹H-NMR (400 MHz, DMSO-D₆) δ_{H}: 1.60-1.70 (4H, two broad signals, Z/E forms), 2.39-2.49 (4H, two broad signals, Z/E forms), 3.45-3.55 (2H, two broad s, Z/E forms), 4.49-4.55 (2H, two broad d, J=7.5 Hz, Z/E forms), 4.92 (2H, broad q, J=7.5 Hz, Z/E forms), 6.91 (1H, superposition of two d, Z/E forms), 7.12 (2H, m, J=8.5 Hz), 7.16 (1H, broad, Z/E forms), 7.35 (2H, broad, Z/E forms), 7.39-7.56 (1H, two broad signals, Z/E forms), 7.64-7.73 (1H, two broad signals, Z/E forms), 8.05-8.15 (1H, two broad signals, Z/E forms), 9.45-9.58 (1H, two broad signals, Z/E forms). MW 476.48. LCMS t_{R} (min): 1.63. MS (APCI), m/z 477.16 [M+H]⁺. HPLC t_{R} (min): 11.55.

### 16. N-{3-[4-(4-Fluoro-benzylamino)-6-(2,2,2-trifluoro-ethoxy)-[1,3,5]triazin-2-ylamino]-phenyl}-acetamide

¹H-NMR (400 MHz, DMSO-D₆) δ_{H}: 2.05 (3H, s), 4.45-4.55 (2H, broad, Z/E forms), 4.95 (2H, broad q, J=7.5 Hz, Z/E forms), 7.15 (4H, broad m), 7.16-7.25 (1H, broad d, J=8.5 Hz, Z/E forms), 7.31-7.42 (2H, broad, Z/E forms), 7.81-8.05 (1H, broad, Z/E forms), 8.09-8.12 (1H, broad, Z/E forms), 9.51-9.60 (1H, broad, Z/E forms), 9.80 (1H, broad). MW 450.40. LCMS t_{R} (min): 1.87. MS (APCI), m/z 451.12 [M+H]⁺. HPLC t_{R} (min): 14.08. M_{P} 231-233° C.

### 17. N-(3,4-Difluoro-phenyl)-N′-(4-fluoro-benzyl)-6-(2,2,2-trifluoro-ethoxy)-[1,3,5]triazine-2,4-diamine

¹H-NMR (400 MHz, DMSO-D₆) δ_{H}: 4.50 (2H, broad), 4.95 (2H, q, J=7.5 Hz), 7.15 (2H, m), 7.33 (4H, broad), 7.75-8.04 (1H, broad, Z/E forms), 8.30 (1H, broad), 9.70-9.80 (1H, broad, Z/E forms). MW 429.33. LCMS t_{R} (min): 2.07. MS (ARCI), m/z 430.10 [M+H]⁺. HPLC t_{R} (min): 16.30. M_{P} 170-172° C.

### 18. N*2*-(4-Fluoro-benzyl)-N*4*-(2-methoxy-pyridin-4-yl)-6-(2,2,2-trifluoro-ethoxy)-1,3,5-triazine-2,4-diamine

¹H-NMR (400 MHz, DMSO-D₆) δ_{H}: 3.82 (3H, s), 4.52 (2H, superposition of two broad d, J=7.5 Hz, Z/E forms), 5.00 (2H, q, J=7.5 Hz), 7.14 (2H, d/d, J=8.5/8.0 Hz), 7.20-7.30 (2H, two broad peaks, Z/E forms), 7.40 (2H, broad peaks, Z/E forms), 7.95 (1H, broad peak, Z/E forms), 8.42 (1H, broad peak, Z/E forms), 9.87-9.95 (1H, two broad peaks, Z/E forms). MW 424.4. LCMS t_{R} (min): 1.75. MS (APCI+), m/z 425.16 [M+H]⁺. HPLC t_{R} (min): 11.79. M_{P} 158-160° C.

### 19. N-(4-Fluoro-benzyl)-N′-(1-methyl-1H-pyrazol-4-yl)-6-(2,2,2-trifluoro-ethoxy)-[1,3,5]triazine-2,4-diamine

¹H-NMR (400 MHz, DMSO-D₆) δ_{H}: 3.71-3.80 (3H, two broad signals, Z/E forms), 4.45-4.55 (2H, two broad signals, Z/E forms), 4.90 (2H, superposition of two broad q, J=7.5 Hz, Z/E forms), 7.11 (2H, broad), 7.35 (2H, broad), 7.42-7.55 (1H, two broad signals, Z/E forms), 7.75-7.92 (1H, two broad signals, Z/E forms), 7.95-8.13 (1H, two broad signals, Z/E forms), 9.41-9.52 (1H, two broad signals, Z/E forms). MW 397.34. LCMS t_{R} (min): 1.81. MS (APCI), m/z 389.13 [M+H]⁺. HPLC t_{R} (min): 13.06. M_{P} 184-186° C.

### 20 N-(4-Fluoro-benzyl)-N′-(1-methanesulfonyl-piperidin-4-yl)-6-(2,2,2-trifluoro-ethoxy)-[1,3,5]triazine-2,4-diamine

¹H-NMR (400 MHz, DMSO-D₆) δ_{H}: 1.50 (2H, broad, Z/E forms), 1.78-1.90 (2H, two broad signals, Z/E forms), 2.75 (2H, m), 2.85 (3H, superposition of two s, Z/E forms), 3.50 (2H, broad, Z/E forms), 3.88 (1H, broad), 4.42 (2H, broad, Z/E forms), 4.88 (2H, broad q, J=7.5 Hz), 7.11 (2H, broad, Z/E forms), 7.31 (2H, broad, Z/E forms), 7.41 (1H, broad, Z/E forms), 7.68-7.95 (1H, broad, Z/E forms). MW 478.47. LCMS t_{R} (min): 1.81. MS (APCI), m/z 479.07 [M+H]⁺. HPLC t_{R} (min): 13.49. M_{P} 166-168° C.

### 21. N-(4-Fluoro-benzyl)-6-(2,2,2-trifluoro-ethoxy)-N′-(1-trifluoromethanesulfonyl-piperidin-4-yl)-[1,3,5]triazine-2,4-diamine

¹H-NMR (400 MHz, DMSO-D₆) δ_{H}: 1.51 (2H, broad, Z/E forms), 1.82-1.95 (2H, two broad signals, Z/E forms), 3.31 (2H, broad, Z/E forms), 3.78 (2H, broad, Z/E forms), 4.00 (1H, broad, Z/E forms), 4.42 (2H, broad, Z/E forms), 4.88 (2H, broad q, J=7.5 Hz, Z/E forms), 7.10 (2H, dd, J=8.5/8.0 Hz), 7.32 (2H, broad, Z/E forms), 7.45 (1H, broad), 7.75-7.93 (1H, two broad signals, Z/E forms). MW 532.44. LCMS t_{R} (min): 2.05. MS (APCI), m/z 533.12 [M+H]⁺. HPLC t_{R} (min): 13.23. M_{P} 186-188° C.

### 22. N-(1-Benzenesulfonyl-piperidin-4-yl)-N′-(4-fluoro-benzyl)-6-(2,2,2-trifluoro-ethoxy)-[1,3,5]triazine-2,4-diamine

Yield 180 mg, 56%. ¹H-NMR (400 MHz, DMSO-D₆) δ_{H}: 1.50 (2H, broad, Z/E forms), 1.75-1.85 (2H, two broad signals, Z/E forms), 2.40 (2H, broad), 3.55 (2H, broad, Z/E forms), 3.70 (1H, broad, Z/E forms), 4.33-4.42 (2H, two broad signals, Z/E forms), 4.82 (2H, q, J=7.5 Hz), 6.99-7.11 (2H, two broad signals, Z/E forms), 7.21-7.29 (2H, two broad signals, Z/E forms), 7.31-7.35 (1H, broad, Z/E forms), 7.65 (2H, broad, Z/E forms), 7.73 (3H, broad), 7.83-7.88 (1H, two broad signals, Z/E forms). MW 540.54. LCMS t_{R} (min): 1.98. MS (APCI), m/z 541.10 [M+H]⁺. HPLC t_{R} (min): 15.03. M_{P} 157-159° C.

### 23. N-[1-(3,4-Difluoro-benzenesulfonyl)-piperidin-4-yl]-N′-(4-fluoro-benzyl)-6-(2,2,2-trifluoro-ethoxy)-[1,3,5]triazine-2,4-diamine

¹H-NMR (400 MHz, DMSO-D₆) δ_{H}: 1.48 (2H, broad, Z/E forms), 1.71-1.85 (2H, broad, Z/E forms), 2.55 (2H, broad, Z/E forms), 3.55 (2H, broad, Z/E forms), 3.68-3.76 (1H, two broad signals, Z/E forms), 4.31-4.42 (2H, two broad signals, Z/E forms), 4.82 (2H, broad q, J=7.5 Hz), 7.11 (2H, broad, Z/E forms), 7.30 (2H, broad), 7.30-7.41 (1H, two broad signals, Z/E forms), 7.65 (1H, broad, Z/E forms), 7.72 (1H, broad, Z/E forms), 7.75-7.88 (1H, two broad signals), 7.90 (1H, broad). MW 576.52. LCMS t_{R} (min): 2.04. MS (APCI), m/z 577.28 [M+H]⁺. HPLC t_{R} (min): 16.32. M_{P} 182-184° C.

### 24. N-{1-[4-(4-Fluoro-benzylamino)-6-(2,2,2-trifluoro-ethoxy)-[1,3,5]triazin-2-yl]-pyrrolidin-3-yl}-methanesulfonamide

Yield 147 mg, 53%. ¹H-NMR (400 MHz, DMSO-D₆) δ_{H}: 1.85 (1H, m), 2.13 (1H, m), 2.92 (3H, s), 3.34 (1H, broad), 3.41-3.58 (2H, two broad signals, Z/E forms), 3.71-3.98 (2H, two broad signals, Z/E forms), 4.40 (2H, broad, Z/E forms), 4.89 (2H, broad q, J=7.5 Hz), 7.09 (2H, broad dd, J=8.5/8.0 Hz), 7.31 (3H, broad), 7.87-7.95 (1H, two broad signals, Z/E forms). MW 464.44. LCMS t_{R} (min): 1.77. MS (APCI), m/z 465.11 [M+H]⁺. HPLC t_{R} (min): 13.03. M_{P} 126-128° C.

### 25. N-{1-[4-(4-Fluoro-benzylamino)-6-(2,2,2-trifluoro-ethoxy)-[1,3,5]triazin-2-yl]-pyrrolidin-3-yl}-benzamide

Yield 55 mg, 19%. ¹H-NMR (400 MHz, DMSO-D₆) δ_{H}: 2.00 (1H, broad, Z/E forms), 2.12 (1H, broad, Z/E forms), 3.50 (2H, broad, Z/E forms), 3.63 (1H, broad, Z/E forms), 3.78 (1H, broad, Z/E forms), 4.41 (2H, broad, Z/E forms), 4.51 (1H, broad, Z/E forms), 4.88 (2H, broad q, J=7.5 Hz), 7.08 (2H, broad, Z/E forms), 7.31 (2H, broad, Z/E forms), 7.43 (2H, t, J=8.5 Hz), 7.51 (1H, t, J=8.5 Hz), 7.83 (2H, d, J=8.5 Hz), 7.85-7.95 (1H, two broad signals, Z/E forms), 8.51 (1H, broad). MW 490.46. LCMS t_{R} (min): 1.90. MS (APCI), m/z 491.13 [M+H]⁺. HPLC t_{R} (min): 14.38. M_{P} 206-208° C.

### 26. 4-Fluoro-N-{1-[4-(4-fluoro-benzylamino)-6-(2,2,2-trifluoro-ethoxy)-[1,3,5]triazin-2-yl]-pyrrolidin-3-yl}-benzamide

Yield 188 mg, 42%. ¹H-NMR (400 MHz, DMSO-D₆) δ_{H}: 1.99 (1H, broad, Z/E forms), 2.18 (1H, broad, Z/E forms), 3.48 (1H, broad, Z/E forms), 3.54 (1H, broad, Z/E forms), 3.63 (1H, broad, Z/E forms), 3.77 (1H, broad, Z/E forms), 4.41 (2H, broad, Z/E forms), 4.50 (1H, broad, Z/E forms), 4.88 (2H, broad q, J=7.5 Hz), 7.08 (2H, broad, Z/E forms), 7.25 (2H, dd, J=8.5/8.0 Hz), 7.32 (2H, broad, Z/E forms), 7.81 (1H, broad), 7.91 (2H, broad m, Z/E forms), 8.50 (1H, broad d, J=7.5 Hz). MW 508.45. LCMS t_{R} (min): 1.93. MS (APCI), m/z 509.14 [M+H]⁺. HPLC t_{R} (min): 14.76. M_{P} 228-230° C.

### 27. [4-(3-Dimethylamino-pyrrolidin-1-yl)-6-(2,2,2-trifluoro-ethoxy)-[1,3,5]triazin-2-yl]-(4-fluoro-benzyl)-amine

¹H-NMR (400 MHz, DMSO-D₆) δ_{H}: 1.70 (1H, m), 2.05 (1H, broad), 2.20 (6H, broad), 2.71 (1H, broad), 3.15 (1H, broad), 3.32 (1H, broad), 3.62 (1H, broad), 3.70 (1H, broad), 4.40 (2H, broad d, J=7.5 Hz), 4.90 (2H, q, J=7.5 Hz), 7.08 (2H, m, J=8.5 Hz), 7.32 (2H, broad), 7.83-7.91 (1H, two broad signals, Z/E forms). MW 414.41. LCMS t_{R} (min): 1.51. MS (APCI), m/z 415.13 [M+H]⁺. HPLC t_{R} (min): 10.35. M_{P} 168-170° C.

### 28. N-(4-Fluoro-benzyl)-N′-phenyl-6-(2,2,2-trifluoro-ethoxy)-[1,3,5]triazine-2,4-diamine

¹H-NMR (400 MHz, DMSO-D₆) δ_{H}: 4.50 (2H, broad, Z/E forms), 4.95 (2H, broad q, J=7.5 Hz, Z/E forms), 6.97 (1H, broad t, J=8.5 Hz, Z/E forms), 7.12 (2H, m, J=8.5 Hz), 7.25 (2H, superposition of two t, J=8.5 Hz, Z/E forms), 7.35 (2H, broad), 7.58-7.70 (2H, broad d, J=8.5 Hz, Z/E forms), 8.18 (1H, broad), 9.50-9.60 (1H, broad, Z/E forms). MW 393.35. LCMS t_{R} (min): 2.01. MS (APCI), m/z 394.09 [M+H]⁺. HPLC t_{R} (min): 15.84. M_{P} 78-80° C.

### 29. 6-[4-(4-Fluoro-benzylamino)-6-(2,2,2-trifluoro-ethoxy)-[1,3,5]triazin-2-ylamino]-4H-benzo[1,4]oxazin-3-one

Yield 110 mg, 24%. ¹H-NMR (400 MHz, DMSO-D₆) δ_{H}: 4.50 (4H, broad), 4.92 (2H, broad q, J=7.5 Hz), 6.81 (1H, broad, Z/E forms), 7.10 (2H, broad, Z/E forms), 7.21-7.31 (1H, two broad signals, Z/E forms), 7.34 (3H, broad), 7.95-8.10 (1H, two broad signals), 9.41-9.55 (1H, two broad signals), 10.70 (1H, broad). MW 464.38. LCMS t_{R} (min): 1.82. MS (APCI), m/z 465.13 [M+H]⁺. HPLC t_{R} (min): 13.65. M_{P} 211-213° C.

### 30. N-(4-Fluoro-benzyl)-N′-(3-[1,2,4]triazol-4-yl-phenyl)-6-(2,2,2-trifluoro-ethoxy)-[1,3,5]triazine-2,4-diamine

¹H-NMR (400 MHz, DMSO-D₆) δ_{H}: 4.51 (2H, broad, Z/E forms), 4.95 (2H, broad q, J=7.5 Hz), 7.06 (1H, broad, Z/E forms), 7.11 (1H, t, J=8.5 Hz), 7.25 (2H, broad), 7.35-7.43 (2H, broad m, Z/E forms), 7.58 (1H, superposition of two d, J=8.5 Hz, Z/E forms), 8.00-8.26 (1H, two broad signals, Z/E forms), 8.28-8.35 (1H, two broad signals, Z/E forms), 8.89 (1H, s), 9.05 (1H, s), 9.85 (1H, broad). MW 460.40. LCMS t_{R} (min): 1.73. MS (APCI), m/z 461.17 [M+H]⁺. HPLC t_{R} (min): 12.93. M_{P} 300-302° C. (decomp).

### 31. 1-{3-[4-(4-Fluoro-benzylamino)-6-(2,2,2-trifluoro-ethoxy)-[1,3,5]triazin-2-ylamino]-phenyl}-pyrrolidin-2-one

¹H-NMR (400 MHz, DMSO-D₆) δ_{H}: 2.00 (2H, broad, Z/E forms), 2.45 (2H, broad, Z/E forms), 3.70-3.80 (2H, two broad t, J=7.5 Hz, Z/E forms), 4.49-4.55 (2H, two broad d, J=7.5 Hz, Z/E forms), 4.95 (2H, broad quartet, J=7.5 Hz, Z/E forms), 7.10 (2H, m, J=8.5 Hz), 7.23 (1H, superposition of two t, J=8.5 Hz, Z/E forms), 7.33 (3H, broad), 7.35-7.48 (1H, two broad, signals, Z/E forms), 7.91-8.15 (1H, two broad signals, Z/E forms), 8.05 (1H, broad), 9.55-9.63 (1H, two broad signals, Z/E forms). MW 476.44. LCMS t_{R} (min): 1.88. MS (APCI), m/z 477.09 [M+H]⁺. HPLC t_{R} (min): 14.42. M_{P} 190-192° C.

### 32. 6-Ethoxy-N-(4-fluoro-benzyl)-N′-(2-methoxy-pyridin-4-yl)-[1,3,5]triazine-2,4-diamine LCMS t_{R} (min): 1.60. MS (APCI+), m/z 371.16 [M+H]⁺.

### 33. (4-Fluoro-benzyl)[4-pyrrolidin-1-yl-6-(2,2,2-trifluoro-ethoxy)-[1,3,5]triazin-2-yl]-amine

¹H-NMR (400 MHz, DMSO-D₆) δ_{H}: 1.87 (4H, broad peak, Z/E forms), 3.42 (4H, broad peak, Z/E forms), 4.42 (2H, d, J=7.5 Hz), 4.88 (2H, q, J=7.5 Hz), 7.10 (2H, d/d, J=8.5/8.0 Hz, Z/E forms), 7.32 (2H, broad peaks, Z/E forms), 7.78-7.85 (1H, two broad peaks, Z/E forms). MW 371.34. LCMS t_{R} (min): 2.02. MS (APCI+), m/z 372.17 [M+H]⁺. HPLC t_{R} (min): 15.58. M_{P} 193-194° C.

### 34. N-(4-Fluoro-benzyl)-N′-(2-fluoro-benzyl)-6-(2,2,2-trifluoro-ethoxy)-[1,3,5]triazine-2,4-diamine

Method M. Yield 80 mg, 21%. ¹H-NMR (400 MHz, DMSO-D₆) δ_{H}: 4.32-4.42 (2H, broad, Z/E forms), 4.51 (2H, broad), 4.89 (2H, broad q, J=7.5 Hz, Z/E forms), 6.98-7.12 (1H, broad, Z/E forms), 7.15 (3H, broad), 7.21-7.32 (1H, broad, Z/E forms), 7.32 (3H, broad), 7.72-7.81 (1H, broad, Z/E forms), 7.92 (1H, broad). MW 425.36. LCMS t_{R} (min): 2.02. MS (APCI), m/z 426.14 [M+H]⁺. HPLC t_{R} (min): 15.64. M_{P} 105-107° C.

### 35. [4-(1,3-Dihydro-isoindol-2-yl)-6-(2,2,2-trifluoro-ethoxy)-[1,3,5]triazin-2-yl]-(4-fluoro-benzyl)-amine

¹H-NMR (400 MHz, DMSO-D₆) δ_{H}: 4.45-4.55 (2H, two broad d, J=7.5 Hz, Z/E forms), 4.80 (4H, broad), 4.95 (2H, q, J=7.5 Hz), 7.15 (2H, broad), 7.31 (2H, broad), 7.40 (4H, broad), 7.95-8.05 (1H, two broad signals, Z/E forms). MW 419.38. LCMS t_{R} (min): 2.16. MS (APCI), m/z 420.06 [M+H]⁺. HPLC t_{R} (min): 17.06. M_{P} 229-231° C.

### 36. (4-Fluoro-benzyl)-[4-(2,2,2-trifluoro-ethoxy)-6-(3-trifluoromethyl-phenoxy)-[1,3,5]triazin-2-yl]-amine

To a solution of compound I-36 (300 mg, 0.89 mmol) in acetonitrile (5 mL) m-trifluoromethyl-phenol (290 mg, 1.79 mmol) and K₂CO₃ (370 mg, 2.68 mmol) were added. The mixture was stirred at refluxing for 5 minutes, cooled to room temperature and diluted with water. The formed solid was collected by filtration, washed with water and diethyl ether and dried giving the compound. Yield 355 mg, 86%. ¹H-NMR (400 MHz, DMSO-D₆) δ_{H}: 4.49-4.25 (2H, two broad d, J=7.5 Hz, Z/E forms), 4.95 (2H, superposition of two q, J=7.5 Hz, Z/E forms), 7.05 (1H, t, J=8.5 Hz), 7.10 (2H, broad), 7.32 (1H, broad), 7.55 (1H, superposition of two d, J=8.5 Hz), 7.65 (3H, broad), 8.75 (1H, broad). MW 462.33. LCMS t_{R} (min): 2.12. MS (APCI), m/z 463.10 [M+H]⁺. HPLC t_{R} (min): 17.03. M_{P} 107-109° C.

### 37. [4-Benzyloxy-6-(2,2,2-trifluoro-ethoxy)-[1,3,5]triazin-2-yl]-(4-fluoro-benzyl)-amine

To a suspension of sodium hydride (605.53 mg, 2.2 mmol) in THF (4 mL) benzyl alcohol (140 mg, 1.29 mmol) was added. The mixture was stirred at room temperature for 3 minutes. Then compound I-36 (300 mg, 0.89 mmol) was added to the obtained solution. The resulting mixture was stirred at room temperature for 1 hour, diluted with water. The formed solid was collected by filtration, washed with water, hexane and ethanol and purified by prepTLC (hexane/ethyl acetate, 6/1) giving the compound. Yield 60 mg, 17%. ¹H-NMR (400 MHz, DMSO-D₆) δ_{H}: 4.50 (2H, broad), 4.95 (2H, broad q, J=7.5 Hz), 5.35 (2H, s), 7.12 (2H, superposition of two m, J=8.5 Hz, Z/E forms), 7.35 (6H, broad), 7.40-7.44 (1H, t, J=8.5 Hz, Z/E forms), 8.60 (1H, broad). MW 408.36. LCMS t_{R} (min): 2.06. MS (APCI), m/z 409.02 [M+H]⁺. HPLC t_{R} (min): 16.39. M_{P} 101-103° C.

### 38. N-(4-Fluoro-benzyl)-N′-(1-isopropyl-pyrrolidin-3-yl)-6-(2,2,2-trifluoro-ethoxy)-[1,3,5]triazine-2,4-diamine

To a solution of compound I-36 (337 mg, 1.0 mmol) in acetonitrile (5 mL) 1-isopropyl-pyrrolidin-3-ylamine (221 mg, 1.1 mmol) and NEt₃ (405 mg, 4.0 mmol) were added dropwise at room temperature. The resulting mixture was stirred at 50° C. for 2 hours and diluted with water. The residue was washed with water and hexane. Purification by column chromatography (silica gel, 50% chloroform/acetone) and prepTLC (chloroform/ethanol, 20/1) gave the compound.

Yield 220 mg, 51%. ¹H-NMR (400 MHz, DMSO-D₆) δ_{H}: 0.95 (6H, broad, Z/E forms), 1.65 (1H, broad, Z/E forms), 2.02 (1H, broad), 2.33 (2H, broad, Z/E forms), 2.58 (2H, broad, Z/E forms), 2.72-2.85 (1H, two broad peaks, Z/E forms), 4.25 (1H, broad, Z/E forms), 4.45 (2H, broad, Z/E forms), 4.87 (2H, broad q, J=7.5 Hz), 6.99 (2H, dd, J=8.5/8.0 Hz), 7.30 (2H, broad m), 7.31-7.39 (1H, two broad peaks, Z/E forms), 7.68-7.88 (1H, two broad peaks, Z/E forms). MW 428.43 LCMS t_{R} (min): 1.56. MS (APCI), m/z 429.21 [M+H]⁺. HPLC t_{R} (min): 11.00. M_{P} 98-99° C.

### Preparation of O,C,N-Triazines in Table 17

### I. Synthesis of Intermediates and Final Compounds

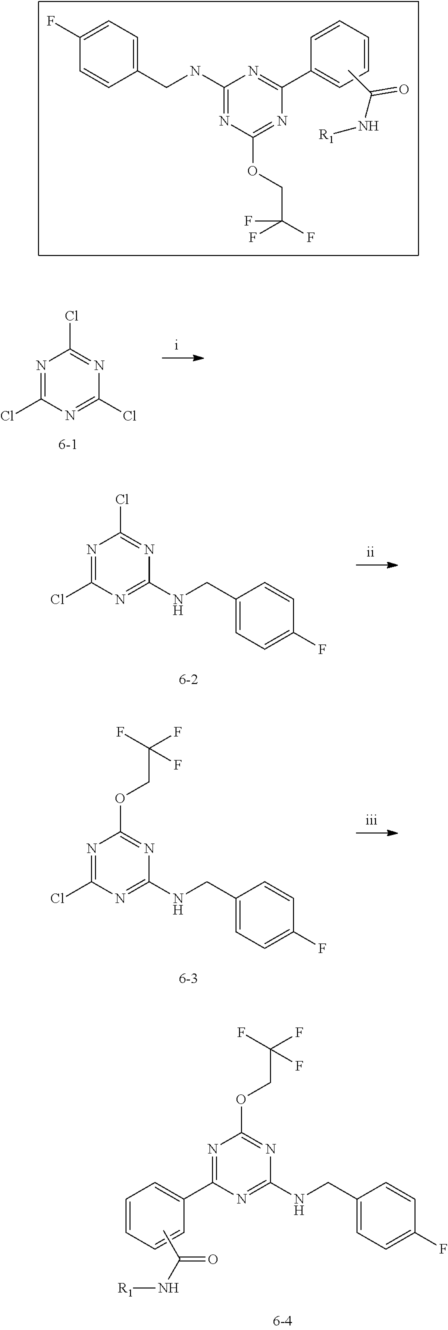

Synthesis of 6-2: In a 1-L flask, 50.0 g (0.27 mol) of cyanuric chloride 6-1 and 24.4 g of sodium acetate were dissolved in 500 mL of dry dioxane. The mixture was cooled to 0 and 34.0 g of 4-fluorobenzylamine in 100 mL of dioxane were added within 30 min under constant stirring. The temperature of the reaction mixture was maintained at 0 □C for 2.5 h till the completion of the reaction (TLC control). The solvent was removed in vacuo. The solids were suspended in CHCl₃, and filtered. The filtrate was concentrated in vacuo, and the precipitate formed was additionally washed with hexane. Yield: 62.2 g, 0.23 mol, 84%. ¹H NMR (DMSO-d₆, 27° C., ppm): d=4.51 d (2H, CH₂), 7.07 t (2H, Ar), 7.34 t (2H, Ar), 9.58 t br. (1H, NH). LC-MS: Not informative due to the lack of ionization.

Synthesis of 6-3: In a 2-L flask, 35.0 g (0.128 mol) of 6-2 were dissolved in 1 L of acetonitrile. The solution was cooled to −30° C. A solution of potassium tert-butoxide (14.4 g, 0.128 mol, 1 eq.) in 2,2,2-trifluoro-1-ethanol (64 g, 0.64 mol, 5 eq.) was added dropwise to the cooled solution of 6-2 during 1.5 h. After complete addition the reaction mixture was allowed to warm to r.t., and reaction was continued for overnight. The solid precipitate was filtered and washed with dry acetonitrile (3×200 mL). The filtrate was concentrated in vacuo producing yellowish oil. To this oil, dry hexane (3×80 mL) was added, the mixture was heated to reflux, and in 3 min the hexane layer was decanted. After three washes, the residue was dried using rotary evaporation, followed by lyophilization. Yield: 32.2 g, 0.10 mol, 75%. ¹H NMR (DMSO-d₆, 90° C., ppm): d=4.53 s (2H, CH₂), 4.95 q (2H, CH₂), 7.10 t (2H, Ar), 7.36 t (2H, Ar), 8.92 s br. (1H, NH). LC-MS: Not informative due to the lack of ionization.

Compound 6-4 was synthesized according to the following procedure: 6-3 (27 mmol), boronic acid (32 mmol), and triphenylphosphine (1.05 g, 4 mmol) were charged into the flask containing 200 mL of dioxane and 30 mL of 2 M aqueous Na₂CO₃ solution. After purging the mixture with Ar for 20 min, [Pd(PPh₃)₄] catalyst (5 mol %) was added. The reaction mixture was heated at 100 □C for 6 h under an Ar atmosphere. After cooling the reaction mixture to rt, the solvent was removed under reduced pressure to produce a yellow oil. The residue was washed with water (20 mL) and extracted with CHCl₃ (20 mL). The organic layer was isolated, and the remaining aqueous portion was washed again with CHCl₃ (3×20 mL). The organic extracts were combined, and solvent was removed in vacuo. The crude product was purified by silica gel chromatography (ca. 120 mL) using CHCl₃-hexanes (3:1 v/v) as an eluent.

TABLE 17

| En | Structure | IUPAC Name | MW | Formula |
|---|---|---|---|---|
| 1 | 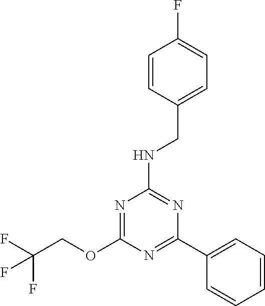 | N-(4-fluorobenzyl)- 4-phenyl-6-(2,2,2- trifluoroethoxy)- 1,3,5-triazin-2-amine | 378.3 | C₁₈H₁₄F₄N₄O |
| 2 | 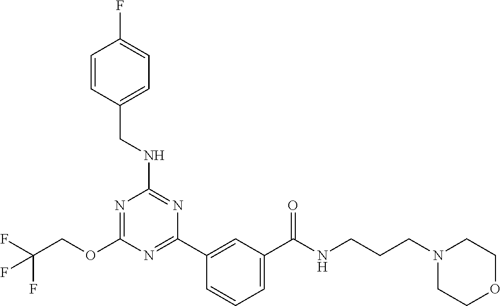 | 3-{4-[(4-fluorobenzyl) amino]-6-(2,2,2- trifluoroethoxy)-1,3,5- triazin-2-yl}-N-[3- (morpholin-4-yl) propyl]benzamide | 548.5 | C₂₆H₂₈F₄N₆O₃ |
| 3 | 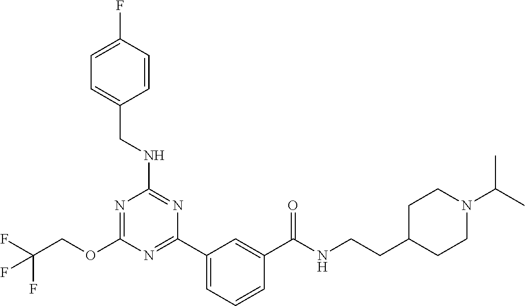 | 3-{4-[(4-fluorobenzyl) amino]-6-(2,2,2- trifluoroethoxy)-1,3,5- triazin-2-yl}-N-{2-[1- (propan-2-yl) piperidin-4-yl]ethyl} benzamide | 574.6 | C₂₉H₃₄F₄N₆O₂ |
| 4 | 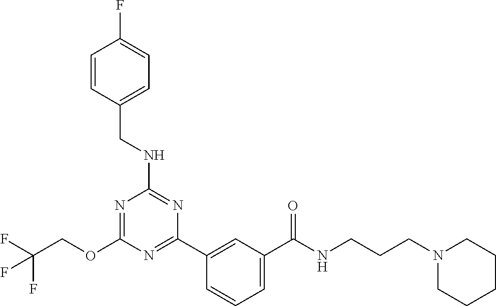 | 3-{4-[(4-fluorobenzyl) amino]-6-(2,2,2- trifluoroethoxy)-1,3,5- triazin-2-yl}-N-[3- (piperidin-1-yl)propyl] benzamide | 546.6 | C₂₇H₃₀F₄N₆O₂ |
| 5 | 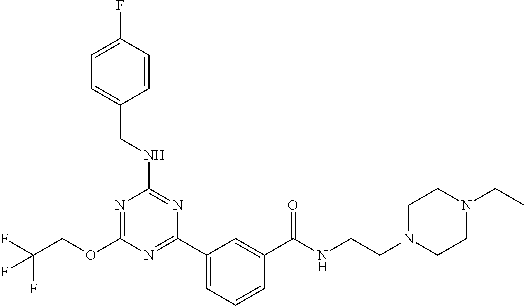 | N-[2-(4-ethyl- piperazin- 1-yl)ethyl]-3-{4-[(4- fluorobenzyl)amino]- 6-(2,2,2- trifluoroethoxy)- 1,3,5-triazin-2- yl}benzamide | 561.6 | C₂₇H₃₁F₄N₇O₂ |
| 6 | 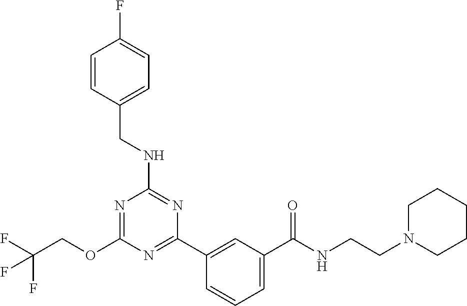 | 3-{4-[(4-fluorobenzyl) amino]-6-(2,2,2- trifluoroethoxy)-1,3,5- triazin-2-yl}-N-[2- (piperidin-1-yl)ethyl] benzamide | 532.5 | C₂₆H₂₈F₄N₆O₂ |
| 7 | 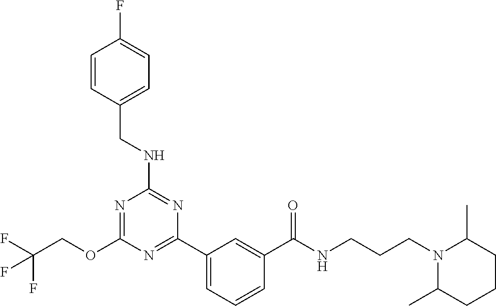 | N-[3-(2,6-dimethyl- piperidin-1-yl) propyl]-3-{4-[(4- fluorobenzyl) amino]-6-(2,2,2- trifluoroethoxy)- 1,3,5-triazin-2- yl}benzamide | 574.6 | C₂₉H₃₄F₄N₆O₂ |
| 8 | 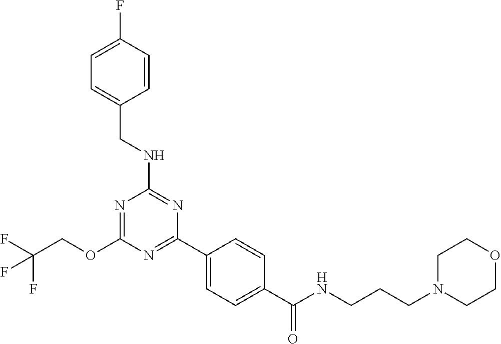 | 4-{4-[(4-fluorobenzyl) amino]-6-(2,2,2- trifluoroethoxy)-1,3,5- triazin-2-yl}-N-[3- (morpholin-4-yl) propyl]benzamide | 548.5 | C₂₆H₂₈F₄N₆O₃ |
| 9 | 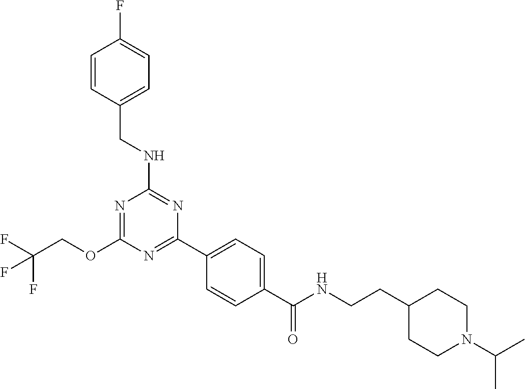 | 4-[4-[(4-fluorobenzyl) amino]-6-(2,2,2- trifluoroethoxy)-1,3,5- triazin-2-yl]-N-[2-(1- isopropylpiperidin-4- yl)ethyl]benzamide | 574.6 | C₂₉H₃₄F₄N₆O₂ |
| 10 | 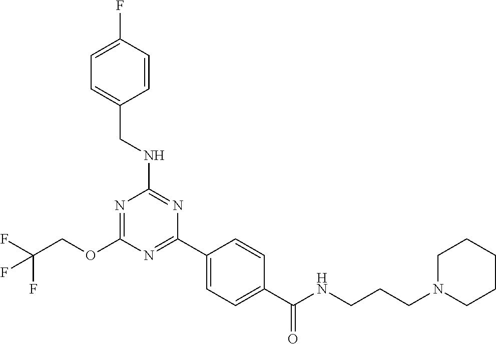 | 4-{4-[(4-fluorobenzyl) amino]-6-(2,2,2- trifluoroethoxy)-1,3,5- triazin-2-yl}-N-[3- (piperidin-1-yl) propyl]benzamide | 546.6 | C₂₇H₃₀F₄N₆O₂ |
| 11 | 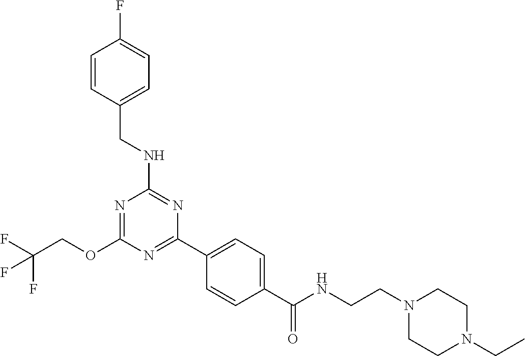 | N-[2-(4-ethyl- piperazin-1-yl) ethyl]-4-{4-[(4- fluorobenzyl)amino]- 6-(2,2,2- trifluoroethoxy)- 1,3,5-triazin-2- yl}benzamide | 561.6 | C₂₇H₃₁F₄N₇O₂ |
| 12 | 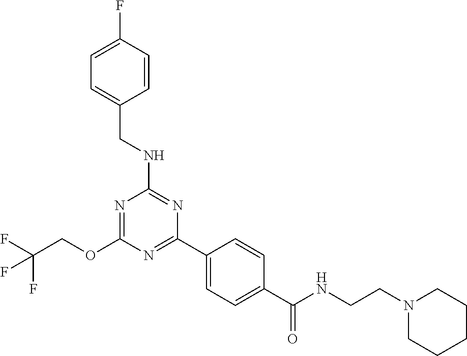 | 4-{4-[(4-fluorobenzyl) amino]-6-(2,2,2- trifluoroethoxy)-1,3,5- triazin-2-yl}-N-[2- (piperidin-1-yl)ethyl] benzamide | 532.5 | C₂₆H₂₈F₄N₆O₂ |
| 13 | 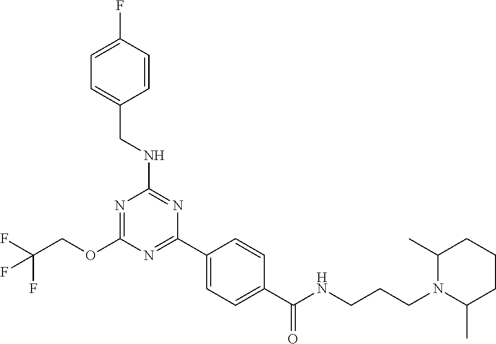 | N-[3-(2,6-dimethyl- piperidin-1-yl)propyl]- 4-{4-[(4-fluorobenzyl) amino]-6-(2,2,2- trifluoroethoxy)-1,3,5- triazin-2- yl}benzamide | 574.6 | C₂₉H₃₄F₄N₆O₂ |
| 14 | 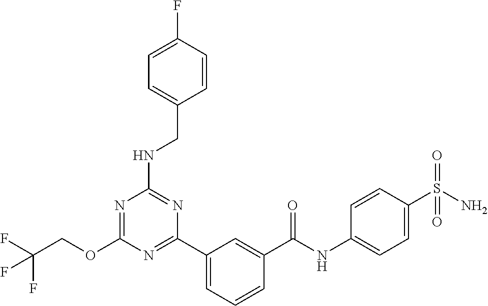 | 3-{4-[(4-fluorobenzyl) amino]-6-(2,2,2- trifluoroethoxy)-1,3,5- triazin-2-yl}-N-(4- sulfamoylphenyl) benzamide | 577.5 | C₂₅H₂₀F₄N₆O₄S |
| 15 | 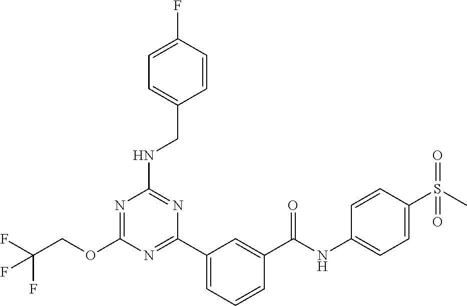 | 3-{4-[(4-fluorobenzyl) amino]-6-(2,2,2- trifluoroethoxy)-1,3,5- triazin-2-yl}-N-[4- (methylsulfonyl) phenyl]benzamide | 576.5 | C₂₆H₂₁F₄N₅O₄S |
| 16 | 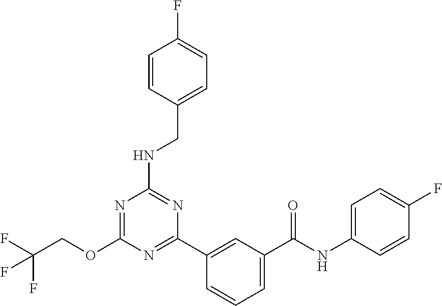 | 3-{4-[(4-fluorobenzyl) amino]-6-(2,2,2- trifluoroethoxy)-1,3,5- triazin-2-yl}-N-(4- fluorophenyl) benzamide | 515.4 | C₂₅H₁₈F₅N₅O₂ |
| 17 | 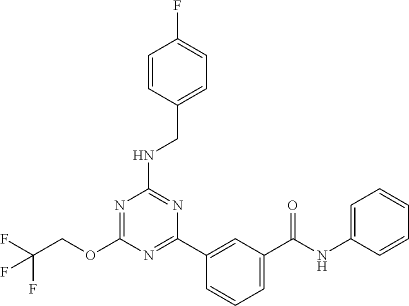 | 3-{4-[(4-fluorobenzyl) amino]-6-(2,2,2- trifluoroethoxy)-1,3,5- triazin-2-yl}-N- phenylbenzamide | 497.4 | C₂₅H₁₉F₄N₅O₂ |
| 18 | 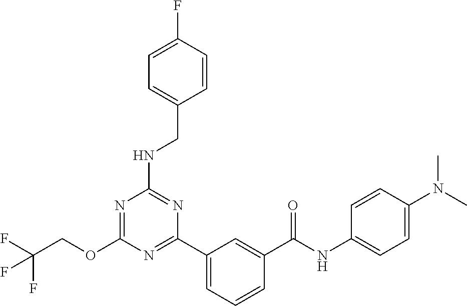 | N-[4-(dimethylamino) phenyl]-3-{4-[(4- fluorobenzyl)amino]- 6-(2,2,2-trifluoro- ethoxy)-1,3,5-triazin- 2-yl}benzamide | 540.5 | C₂₇H₂₄F₄N₆O₂ |
| 19 | 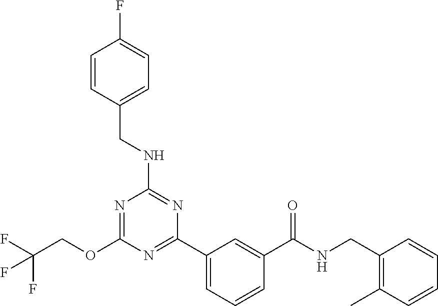 | 3-{4-[(4-fluorobenzyl) amino]-6-(2,2,2- trifluoroethoxy)-1,3,5- triazin-2-yl}-N-(2- methylbenzyl) benzamide | 525.5 | C₂₇H₂₃F₄N₅O₂ |
| 20 | 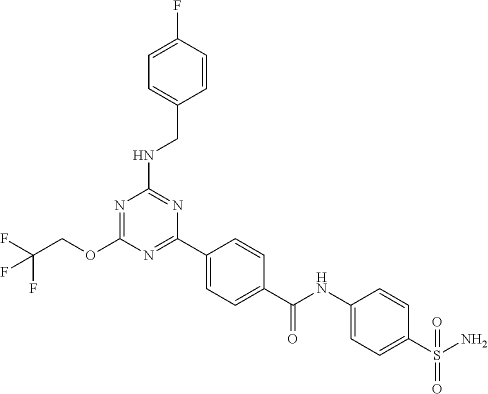 | N-[4-(aminosulfonyl) phenyl]-4-[4-[(4- fluorobenzyl)amino]- 6-(2,2,2-trifluoro- ethoxy)-1,3,5-triazin- 2-yl]benzamide | 577.5 | C₂₅H₂₀F₄N₆O₄S |
| 21 | 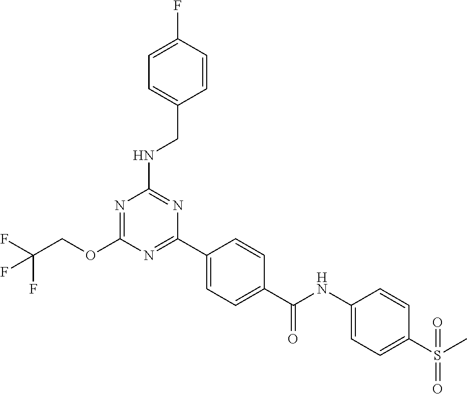 | 4-{4-[(4-fluorobenzyl) amino]-6-(2,2,2- trifluoroethoxy)-1,3,5- triazin-2-yl}-N-[4- (methylsulfonyl) phenyl]benzamide | 576.5 | C₂₆H₂₁F₄N₅O₄S |
| 22 | 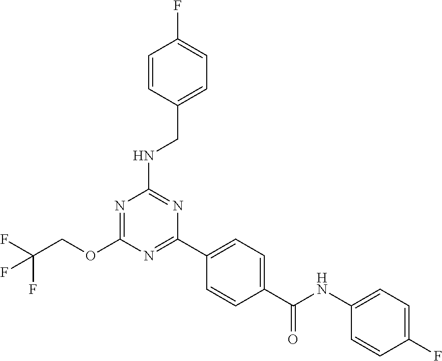 | 4-[4-[(4-fluorobenzyl) amino]-6-(2,2,2- trifluoroethoxy)- 1,3,5-triazin-2-yl]-N- (4-fluorophenyl) benzamide | 515.4 | C₂₅H₁₈F₅N₅O₂ |
| 23 | 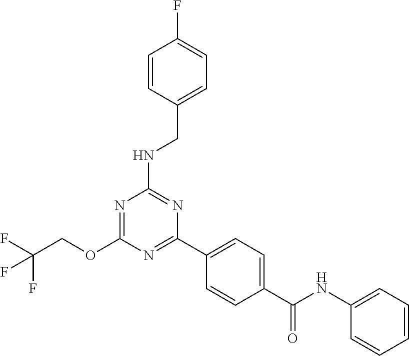 | 4-[4-[(4-fluorobenzyl) amino]-6-(2,2,2- trifluoroethoxy)-1,3,5- triazin-2-yl]-N- phenylbenzamide | 497.4 | C₂₅H₁₉F₄N₅O₂ |
| 24 | 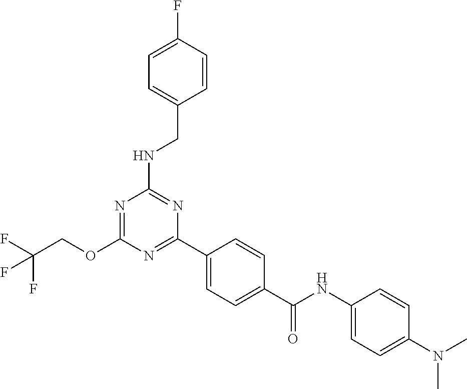 | N-[4-(dimethylamino) phenyl]-4-[4-[(4- fluorobenzyl)amino]- 6-(2,2,2-trifluoro- ethoxy)-1,3,5-triazin- 2-yl]benzamide | 540.5 | C₂₇H₂₄F₄N₆O₂ |
| 25 | 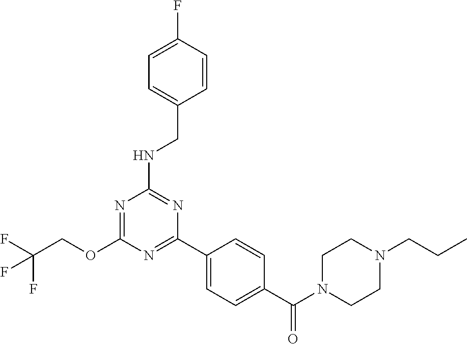 | (4-{4-[(4- fluorobenzyl) amino]-6-(2,2,2- trifluoroethoxy)- 1,3,5-triazin-2- yl}phenyl)(4- propylpiperazin-1- yl)methanone | 532.5 | C₂₆H₂₈F₄N₆O₂ |
| 26 | 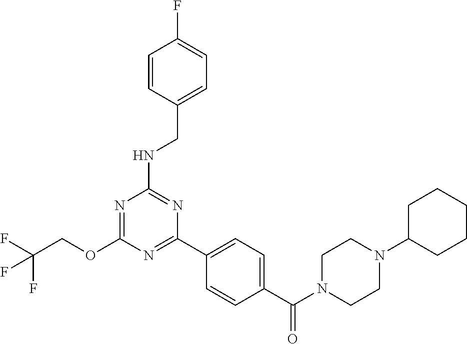 | (4-cyclohexyl- piperazin- 1-yl)(4-{4-[(4- fluorobenzyl)amino]- 6-(2,2,2- trifluoroethoxy)- 1,3,5-triazin-2- yl}phenyl)methanone | 572.6 | C₂₉H₃₂F₄N₆O₂ |
| 27 | 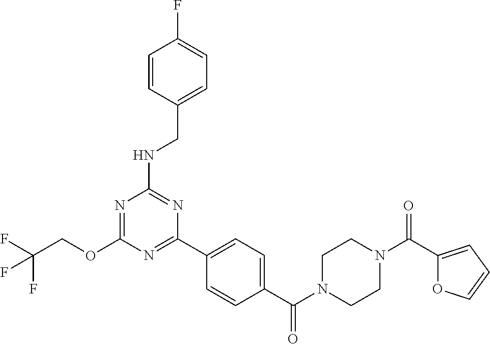 | (4-{4-[(4- fluorobenzyl) amino]-6-(2,2,2- trifluoroethoxy)- 1,3,5-triazin-2-yl} phenyl)[4- (furan-2-ylcarbonyl) piperazin-1-yl] methanone | 584.5 | C₂₈H₂₄F₄N₆O₄ |
| 28 | 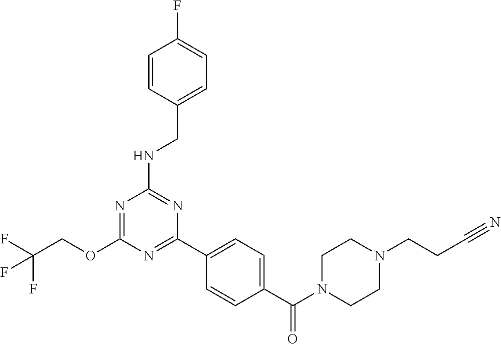 | 3-{4-[(4-{4-[(4- fluorobenzyl)amino]- 6-(2,2,2- trifluoroethoxy)- 1,3,5-triazin-2- yl}phenyl)carbonyl] piperazin-1- yl}propanenitrile | 543.5 | C₂₆H₂₅F₄N₇O₂ |
| 29 | 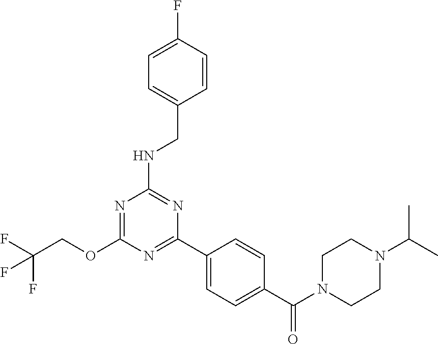 | (4-{4-[(4- fluorobenzyl) amino]-6-(2,2,2- trifluoroethoxy)- 1,3,5-triazin-2- yl}phenyl)[4- (propan-2-yl) piperazin-1- yl]methanone | 532.5 | C₂₆H₂₈F₄N₆O₂ |
| 30 | 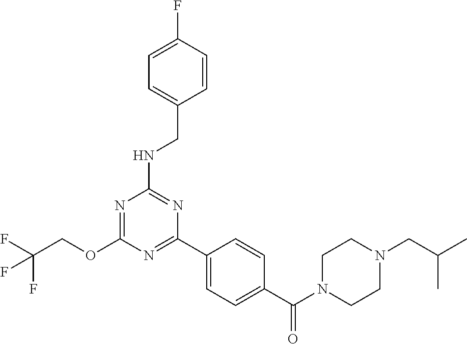 | (4-{4-[(4- fluorobenzyl) amino]-6-(2,2,2- trifluoroethoxy)- 1,3,5-triazin-2- yl}phenyl)[4-(2- methylpropyl) piperazin-1-yl] methanone | 546.6 | C₂₇H₃₀F₄N₆O₂ |
| 31 | 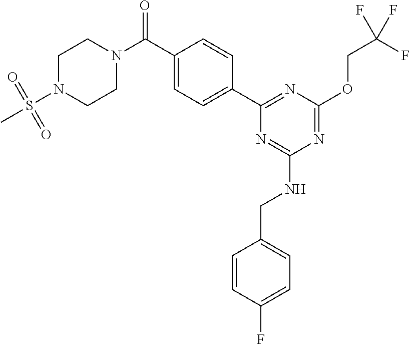 | N-(4-fluorobenzyl)- 4-(4-{[4- (methylsulfonyl) piperazin-1-yl] carbonyl}phenyl)-6- (2,2,2- trifluoroethoxy)- 1,3,5-triazin-2- amine | 568.6 | C₂₄H₂₄F₄N₆O₄S |
| 32 | 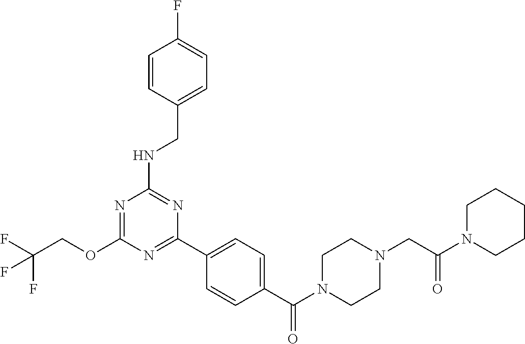 | 2-{4-[(4-{4-[(4- fluorobenzyl) amino]-6- (2,2,2-trifluoro- ethoxy)- 1,3,5-triazin-2- yl}phenyl)carbonyl] piperazin-1-yl}-1- (piperidin-1-yl) ethanone | 615.6 | C₃₀H₃₃F₄N₇O₃ |
| 33 | 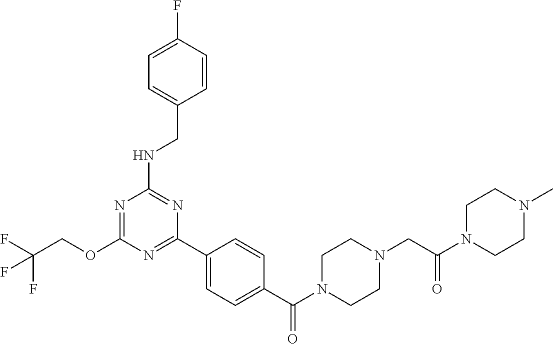 | 2-{4-[(4-{4-[(4- fluorobenzyl)amino]- 6-(2,2,2- trifluoroethoxy)- 1,3,5-triazin-2-yl} phenyl)carbonyl] piperazin-1-yl}-1-(4- methylpiperazin-1- yl)ethanone | 630.6 | C₃₀H₃₄F₄N₈O₃ |
| 34 | 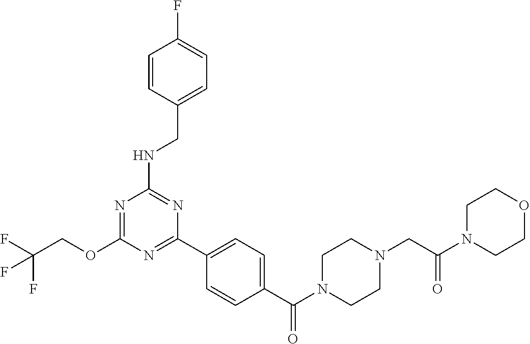 | 2-{4-[(4-{4-[(4- fluorobenzyl)amino]- 6-(2,2,2-tri- fluoroethoxy)- 1,3,5-triazin-2-yl} phenyl)carbonyl]- piperazin-1-yl}-1- (morpholin-4-yl) ethanone | 617.6 | C₂₉H₃₁F₄N₇O₄ |
| 35 | 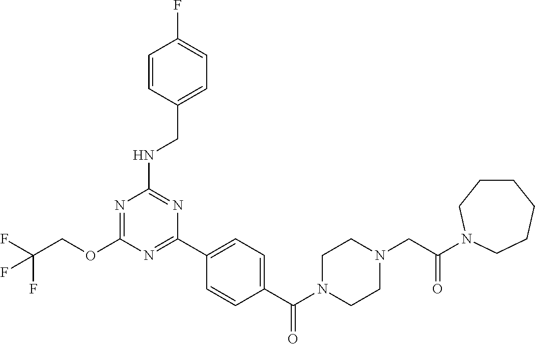 | 1-(azepan-1-yl)-2- {4-[(4-{4-[(4- fluorobenzyl)-amino]- 6-(2,2,2- trifluoroethoxy)-1,3,5- triazin-2-yl}phenyl) carbonyl]piperazin-1- yl}ethanone | 629.7 | C₃₁H₃₅F₄N₇O₃ |
| 36 | 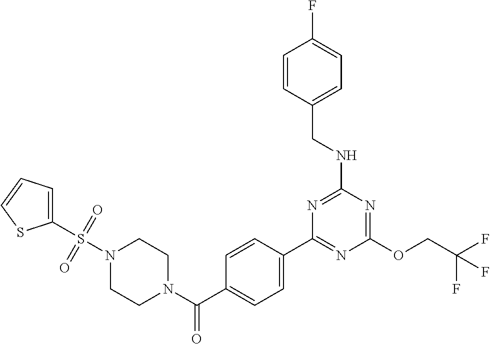 | (4-{4-[(4- fluorobenzyl) amino]-6-(2,2,2- trifluoroethoxy)- 1,3,5-triazin-2- yl}phenyl)[4- (thiophen-2- ylsulfonyl) piperazin-1-yl] methanone | 637.7 | C₂₇H₂₄F₄N₆O₄S₂ |
| 37 | 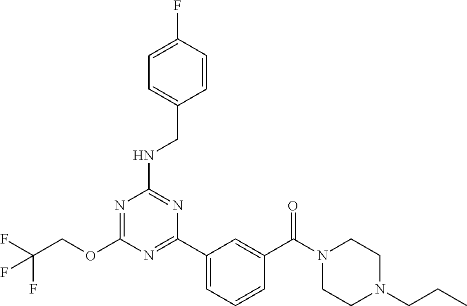 | (3-{4-[(4- fluorobenzyl) amino]-6-(2,2,2- trifluoroethoxy)- 1,3,5-triazin-2- yl}phenyl)(4- propylpiperazin-1- yl)methanone | 532.5 | C₂₆H₂₈F₄N₆O₂ |
| 38 | 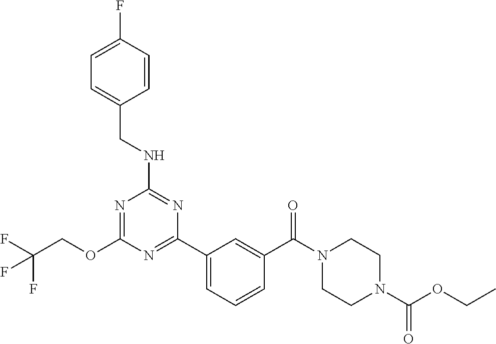 | ethyl 4-[(3-{4-[(4- fluorobenzyl)amino]- 6-(2,2,2- trifluoroethoxy)- 1,3,5-triazin-2- yl}phenyl)carbonyl] piperazine-1- carboxylate | 562.5 | C₂₆H₂₆F₄N₆O₄ |
| 39 | 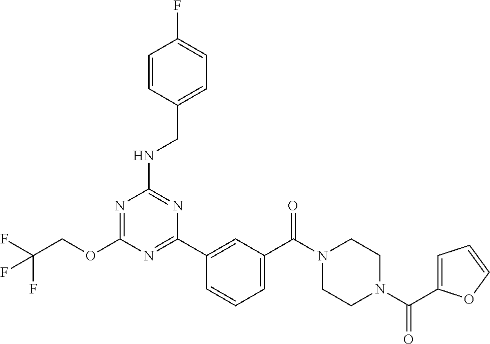 | (3-{4-[(4- fluorobenzyl) amino]-6-(2,2,2- trifluoroethoxy)- 1,3,5-triazin-2- yl}phenyl)[4-(furan- 2-ylcarbonyl) piperazin-1- yl]methanone | 584.5 | C₂₈H₂₄F₄N₆O₄ |
| 40 | 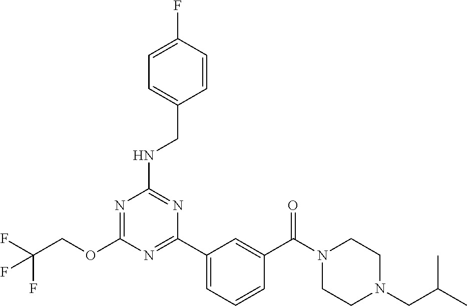 | (3-{4-[(4- fluorobenzyl) amino]-6-(2,2,2- trifluoroethoxy)- 1,3,5-triazin-2- yl}phenyl)[4-(2- methylpropyl) piperazin-1-yl] methanone | 546.6 | C₂₇H₃₀F₄N₆O₂ |
| 41 | 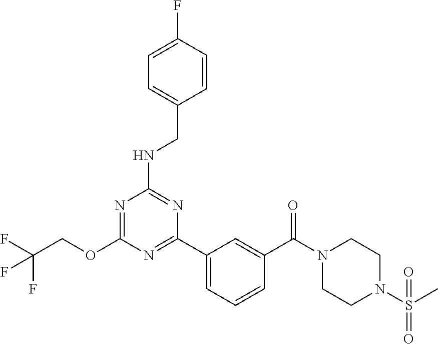 | (3-{4-[(4- fluorobenzyl) amino]-6-(2,2,2- trifluoroethoxy)- 1,3,5-triazin-2-yl} phenyl)[4- (methylsulfonyl) piperazin-1-yl] methanone | 569.6 | C₂₄H₂₄F₄N₆O₄S |
| 42 | 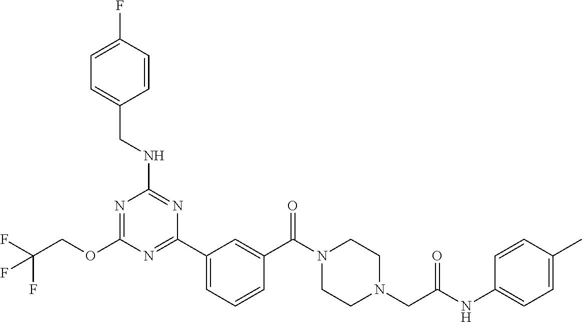 | 2-{4-[(3-{4-[(4- fluorobenzyl)amino]- 6-(2,2,2- trifluoroethoxy)- 1,3,5-triazin-2-yl} phenyl)carbonyl]- piperazin-1-yl}-N- (4-methylphenyl) acetamide | 637.6 | C₃₂H₃₁F₄N₇O₃ |
| 43 | 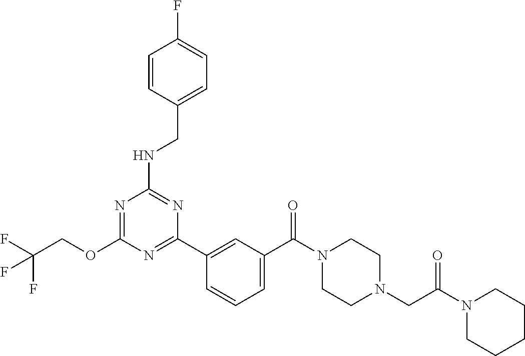 | 2-{4-[(3-{4-[(4- fluorobenzyl)amino]- 6-(2,2,2- trifluoroethoxy)- 1,3,5-triazin-2-yl} phenyl)carbonyl]- piperazin-1-yl}-1- (piperidin-1-yl) ethanone | 615.6 | C₃₀H₃₃F₄N₇O₃ |
| 44 | 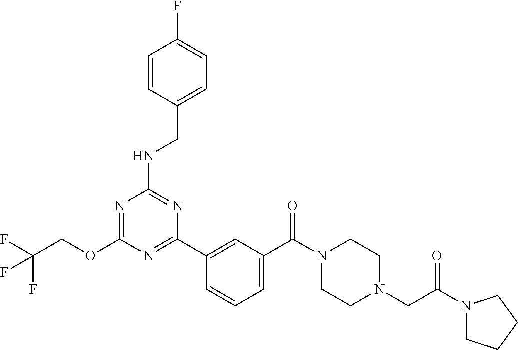 | 2-{4-[(3-{4-[(4- fluorobenzyl)amino]- 6-(2,2,2- trifluoroethoxy)- 1,3,5-triazin-2- yl}phenyl)carbonyl]- piperazin-1-yl}-1- (pyrrolidin-1-yl) ethanone | 601.6 | C₂₉H₃₁F₄N₇O₃ |
| 45 | 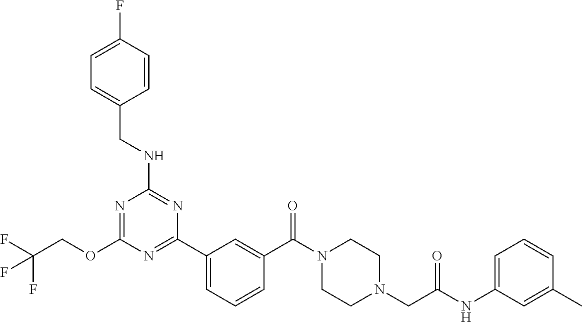 | 2-{4-[(3-{4-[(4- fluorobenzyl)amino]- 6-(2,2,2- trifluoroethoxy)- 1,3,5-triazin-2- yl}phenyl)carbonyl]- piperazin-1-yl}-N- (3-methylphenyl) acetamide | 637.6 | C₃₂H₃₁F₄N₇O₃ |
| 46 | 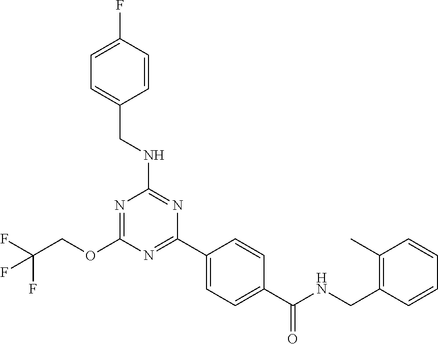 | 4-(4-(4-fluorobenzyl- amino)-6-(2,2,2- trifluoroethoxy)- 1,3,5-triazin-2-yl)- N-(2-methylbenzyl) benzamide | 525.5 | C₂₇H₂₃F₄N₅O₂ |

### Procedures and Analytical Data for Table 17.

### 1. (4-Fluoro-benzyl)-[4-phenyl-6-(2,2,2-trifluoro-ethoxy)-[1,3,5]triazin-2-yl]-amine

A mixture of compound I-36 (505 mg, 1.5 mmol), phenyl boronic acid (182 mg, 1.5 mmol), Pd(PPh₃)₄ (0.03 mmol, 34.7 mg), Na₂CO₃ (318 mg), dioxane (3 mL) and water (3 mL) was stirred at refluxing for 6 hours, cooled down to room temperature and diluted with cold water. The formed solid was collected by filtration, filtered through Celite and purified by column chromatography (dichloromethane/hexane) and recrystallized from ethyl acetate/hexane giving the compound (60 mg, 11%). ¹H-NMR (400 MHz, DMSO-D₆) δ_{H}: 4.55-4.65 (2H, two d, J=7.5 Hz, Z/E forms), 5.17 (2H, q, J=7.5 Hz), 7.12 (2H, d/d, J=8.5/8.0 Hz), 7.39 (2H, broad, Z/E forms), 7.50 (2H, t, J=8.5 Hz), 7.57 (1H, t, J=8.5 Hz), 8.31 (2H, superposition of two d, J=8.5 Hz, Z/E forms), 8.65-8.78 (1H, two broad signals, Z/E forms). MW 378.33. LCMS t_{R} (min): 2.11. MS (APCI), m/z 379.05 [M+H]⁺. HPLC t_{R} (min): 16.92. M_{P} 170-172° C.

### 2. 3-{4-[(4-fluorobenzyl)amino]-6-(2,2,2-trifluoroethoxy)-1,3,5-triazin-2-yl}-N-[3-(morpholin-4-yl)propyl]benzamide

1H NMR (DMSO-d6, 90° C., ppm): d=1.75 m (2H, CH2), 2.34-2.44 m (6H, 3CH2), 3.37 q (2H, CH2), 3.58 t (4H, 2CH2), 4.64 s br. (2H, CH2), 5.06 q (2H, CH2), 7.11 t (2H, Ar), 7.43 t (2H, Ar), 7.59 t (1H, Ar), 8.01 d (1H, Ar), 8.29 s br. (1H, NH), 8.38-8.62 m br. (2H, Ar and NH), 8.75 s (1H, Ar). LC-MS [M+1]: calc'd: 549.5; obs'd: 549.6

### 3. 3-{4-[(4-fluorobenzyl)amino]-6-(2,2,2-trifluoroethoxy)-1,3,5-triazin-2-yl}-N-{2-[1-(propan-2-yl)piperidin-4-yl]ethyl}benzamide

1H NMR (DMSO-d6, 90° C., ppm): d=1.00 d (6H, 2CH3), 1.09-1.45 m br. (4H, 2CH2), 1.53 q (2H, CH2), 1.73 d (2H, CH2), 2.18 t (2H, CH2), 2.63-3.02 m (2H, 2CH), 3.35 q (2H, CH2), 4.64 s br. (2H, CH2), 5.06 q (2H, CH2), 7.11 t (2H, Ar), 7.43 t (2H, Ar), 7.58 t (1H, Ar), 8.01 d (1H, Ar), 8.21 s br. (1H, NH), 8.44 d (1H, Ar), 8.50 s br. (1H, NH), 8.75 s (1H, Ar). LC-MS [M+1]: calc'd: 575.6; obs'd: 575.7

### 4. 3-{4-[(4-fluorobenzyl)amino]-6-(2,2,2-trifluoroethoxy)-1,3,5-triazin-2-yl}-N-[3-(piperidin-1-yl)propyl]benzamide

1H NMR (DMSO-d6, 90° C., ppm): d=1.39 q (2H, CH2), 1.50 m (4H, 2CH2), 1.73 m (2H, CH2), 2.32-2.39 m (6H, 3CH2), 3.35 q (2H, CH2), 4.64 s br. (2H, CH2), 5.06 q (2H, CH2), 6.99 s (2H, CH2), 7.11 t (2H, Ar), 7.43 t br. (2H, Ar), 7.58 m (2H, Ar), 8.00 d (1H, Ar), 8.32 s br. (1H, NH), 8.44 d (1H, Ar), 8.53 s br. (1H, NH), 8.74 s (1H, Ar). LC-MS [M+1]: calc'd: 547.6; obs'd: 547.6.

### 5. N-[2-(4-ethylpiperazin-1-yl)ethyl]-3-{4-[(4-fluorobenzyl)amino]-6-(2,2,2-trifluoroethoxy)-1,3,5-triazin-2-yl}benzamide

1H NMR (DMSO-d6, 90° C., ppm): d=0.99 t (3H, CH3), 2.24-2.44 m (6H, 3CH2), 2.44-2.58 m (6H, 3CH2), 3.43 q (2H, CH2), 4.64 s br. (2H, CH2), 5.06 q (2H, CH2), 7.11 t (2H, Ar), 7.43 t (2H, Ar), 7.59 t (1H, Ar), 8.00 d (1H, Ar), 8.13 m (1H, Ar), 8.44 d (1H, Ar), 8.52 s br. (1H, NH), 8.75 s (1H, NH). LC-MS [M+1]: calc'd: 562.6; obs'd: 562.5.

### 6. 3-{4-[(4-fluorobenzyl)amino]-6-(2,2,2-trifluoroethoxy)-1,3,5-triazin-2-yl}-N-[2-(piperidin-1-yl)ethyl]benzamide

1H NMR (DMSO-d6, 90° C., ppm): d=1.42 d (2H, CH2), 1.54 t (4H, 2CH2), 2.49 s br. (4H, 2CH2), 2.58 t (2H, CH2), 3.44 q (2H, CH2), 4.63 s br. (2H, CH2), 5.06 q (2H, CH2), 7.10 t (2H, Ar), 7.42 t (2H, Ar), 7.58 t (1H, Ar), 8.00 t (1H, Ar), 8.16 s br. (1H, Ar), 8.44 d (1H, Ar), 8.52 s br. (1H, NH), 8.75 s (1H, Ar). LC-MS [M+1]: calc'd: 533.5; obs'd: 533.4.

### 7. N-[3-(2,6-dimethylpiperidin-1-yl)propyl]-3-{4-[(4-fluorobenzyl)amino]-6-(2,2,2-trifluoroethoxy)-1,3,5-triazin-2-yl}benzamide

1H NMR (DMSO-d6, 90° C., ppm): d=1.05 d (6H, 2CH3), 1.11-1.36 m (4H, 2CH2), 1.45-1.80 m (5H, 2CH2 & CH), 2.30-2.55 m (1H, CH), 2.72 t (2H, CH2), 3.28 q (2H, CH2), 4.64 s br. (2H, CH2), 5.06 q (2H, CH2), 7.11 t (2H, Ar), 7.43 t (2H, Ar), 7.58 t (1H, Ar), 8.00 d (1H, Ar), 8.33 m (1H, Ar), 8.44 d (1H, Ar), 8.51 s br. (1H, NH), 8.75 s (1H, NH). LC-MS [M+1]: calc'd: 575.6; obs'd: 575.5.

### 8. 4-{4-[(4-fluorobenzyl)amino]-6-(2,2,2-trifluoroethoxy)-1,3,5-triazin-2-yl}-N-[3-(morpholin-4-yl)propyl]benzamide

1H NMR (DMSO-d6, 90° C., ppm): d=1.74 m (2H, CH2), 2.34-2.45 m (6H, 3CH2), 3.36 q (2H, CH2), 3.59 t (4H, 2CH2), 4.64 s br. (2H, CH2), 5.06 q (2H, CH2), 7.11 t (2H, Ar), 7.42 t (2H, Ar), 7.94 d (2H, Ar), 8.26 m br. (1H, NH), 8.38 d (2H, Ar), 8.49 s br. (1H, NH). LC-MS [M+1]: calc'd: 549.5; obs'd: 549.6.

### 9. 4-[4-[(4-fluorobenzyl)amino]-6-(2,2,2-trifluoroethoxy)-1,3,5-triazin-2-yl]-N-[2-(1-isopropylpiperidin-4-yl)ethyl]benzamide

1H NMR (DMSO-d6, 90° C., ppm): d=1.01 d (6H, 2CH3), 1.11-1.46 m br. (2H, 2CH2), 1.53 q (2H, CH2), 1.74 d (2H, CH2), 2.22 t (2H, CH2), 2.67-3.00 m (3H, CH and CH2), 3.34 q (2H, CH2), 4.63 s br. (2H, CH2), 5.05 q (2H, CH2), 7.11 t (2H, Ar), 7.42 t (2H, Ar), 7.94 d (2H, Ar), 8.20 s br. (1H, NH), 8.37 d (2H, Ar), 8.50 s br. (1H, NH). LC-MS [M+1]: calc'd: 575.6; obs'd: 575.7.

### 10. 4-{4-[(4-fluorobenzyl)amino]-6-(2,2,2-trifluoroethoxy)-1,3,5-triazin-2-yl}-N-[3-(piperidin-1-yl)propyl]benzamide

1H NMR (DMSO-d6, 90° C., ppm): d=1.59 s br. (2H, CH2), 1.78 s br. (4H, 2CH2), 1.99 m (2H, CH2), 3.12 t (6H, 3CH2), 3.40 q (2H, CH2), 4.64 s br. (2H, CH2), 5.06 q (2H, CH2), 7.12 t (2H, Ar), 7.43 t (2H, Ar), 7.97 d (2H, Ar), 8.40 d (2H, Ar), 8.43-8.66 (1H, NH). LC-MS [M+1]: calc'd: 547.6; obs'd: 547.6.

### 11. N-[2-(4-ethylpiperazin-1-yl)ethyl]-4-{4-[(4-fluorobenzyl)amino]-6-(2,2,2-trifluoroethoxy)-1,3,5-triazin-2-yl}benzamide

1H NMR (DMSO-d6, 90° C., ppm): d=1.00 t (3H, CH3), 2.27-2.43 m (6H, 3CH2), 2.43-2.60 m (6H, 3CH2), 3.42 q (2H, CH2), 4.64 s br. (2H, CH2), 5.06 q (2H, CH2), 7.11 t (2H, Ar), 7.42 t (2H, Ar), 7.93 d (2H, Ar), 8.11 t br. (1H, NH), 8.38 d (2H, Ar), 8.48 s br. (1H, NH). LC-MS [M+1]: calc'd: 562.6; obs'd: 562.5.

### 12. 4-{4-[(4-fluorobenzyl)amino]-6-(2,2,2-trifluoroethoxy)-1,3,5-triazin-2-yl}-N-[2-(piperidin-1-yl)ethyl]benzamide

1H NMR (DMSO-d6, 90° C., ppm): d=1.06 d (6H, 2CH3), 1.09-1.39 m (4H, 2CH2), 1.46-1.80 m (5H, 2CH2 & CH), 2.38-2.56 m (1H, CH), 2.72 t (2H, CH2), 3.28 q (2H, CH2), 4.63 s br. (2H, CH2), 5.06 q (2H, CH2), 7.11 t (2H, Ar), 7.42 t (2H, Ar), 7.94 d (2H, Ar), 8.30 s (1H, NH), 8.38 d (2H, Ar), 8.49 s br. (1H, NH). LC-MS [M+1]: calc'd: 575.6; obs'd: 575.7.

### 13. N-[3-(2,6-dimethylpiperidin-1-yl)propyl]-4-{4-[(4-fluorobenzyl)amino]-6-(2,2,2-trifluoroethoxy)-1,3,5-triazin-2-yl}benzamide

LCMS: M+1=574.6; 1H NMR (DMSO-d6, 90° C., ppm): =1.11 s (6H), 1.25 m (4H), 1.61 m (4H), 2.48 m (2H), 4.68 s (2H), 4.99 m (2H), 7.15 m (2H), 7.42 m (2H), 8.19 dd (4H), 8.29 m (1H), 8.49 s (1H).

### 14. 3-{4-[(4-fluorobenzyl)amino]-6-(2,2,2-trifluoroethoxy)-1,3,5-triazin-2-yl}-N-(4-sulfamoylphenyl)benzamide

1H NMR (DMSO-d6, 90° C., ppm): d=4.64 s br. (2H, CH2), 5.08 q (2H, CH2), 6.97 s (2H, Ar), 7.11 t (2H, Ar), 7.44 t (2H, Ar), 7.68 t (1H, Ar), 7.84 d (2H, Ar), 7.95 d (2H, Ar), 8.17 d (1H, Ar), 8.41-8.71 m (2H, Ar & NH), 8.87 s (1H, NH), 10.46 s (1H, NH). LC-MS [M+1]: calc'd: 577.5; obs'd: 577.5.

### 15. 3-{4-[(4-fluorobenzyl)amino]-6-(2,2,2-trifluoroethoxy)-1,3,5-triazin-2-yl}-N-[4-(methylsulfonyl)phenyl]benzamide

1H NMR (DMSO-d6, 90° C., ppm): d=3.15 s (3H, CH3), 4.64 s br. (2H, CH2), 5.08 q (2H, CH2), 7.11 t (2H, Ar), 7.44 t (2H, Ar), 7.69 t (1H, Ar), 7.92 d (2H, Ar), 8.06 d (2H, Ar), 8.18 d (1H, Ar), 8.34-8.76 m (2H, Ar & NH), 8.88 s (1H, NH), 10.58 s (1H, NH). LC-MS [M+1]: calc'd: 576.5; obs'd: 576.4.

### 16. 3-{4-[(4-fluorobenzyl)amino]-6-(2,2,2-trifluoroethoxy)-1,3,5-triazin-2-yl}-N-(4-fluorophenyl)benzamide

1H NMR (DMSO-d6, 90° C., ppm): d=4.65 s br. (2H, CH2), 5.08 q (2H, CH2), 7.00-7.26 m (4H, Ar), 7.44 t (2H, Ar), 7.66 t (1H, Ar), 7.79 t (2H, Ar), 8.14 d (1H, Ar), 8.30-8.72 m br. (2H, Ar and NH), 8.86 s (1H, Ar), 10.19 s (1H, NH). LC-MS [M+1]: calc'd: 516.4; obs'd: 516.4.

### 17. 3-{4-[(4-fluorobenzyl)amino]-6-(2,2,2-trifluoroethoxy)-1,3,5-triazin-2-yl}-N-phenylbenzamide

1H NMR (DMSO-d6, 90° C., ppm): d=4.64 s br. (2H, CH2), 5.08 q (2H, CH2), 7.05-7.18 m (3H, Ar), 7.36 t (2H, Ar), 7.44 t (2H, Ar), 7.66 t (1H, Ar), 7.78 d (2H, Ar), 8.15 d (1H, Ar), 8.51 d (1H, Ar), 8.57 s br. (1H, NH), 8.87 s (1H, Ar), 10.13 s (1H, NH). LC-MS [M+1]: calc'd: 498.5; obs'd: 498.4.

### 18. N-[4-(dimethylamino)phenyl]-3-{4-[(4-fluorobenzyl)amino]-6-(2,2,2-trifluoroethoxy)-1,3,5-triazin-2-yl}benzamide

1H NMR (DMSO-d6, 90° C., ppm): d=2.89 s (6H, 2CH3), 4.64 s br. (2H, CH2), 5.08 q (2H, CH2), 6.75 d (2H, Ar), 7.11 t (2H, Ar), 7.43 t (2H, Ar), 7.49-7.71 m (3H, Ar), 8.12 d (1H, Ar), 8.35-8.67 m br. (2H, Ar and NH), 8.85 s (1H, Ar), 9.85 s (1H, NH). LC-MS [M+1]: calc'd: 541.5; obs'd: 541.4.

### 19. 3-{4-[(4-fluorobenzyl)amino]-6-(2,2,2-trifluoroethoxy)-1,3,5-triazin-2-yl}-N-(2-methylbenzyl)benzamide

1H NMR (DMSO-d6, 90° C., ppm): d=2.37 s (3H, CH3), 4.53 d (2H, CH2), 4.63 s br. (2H, CH2), 5.06 q (2H, CH2), 7.01-7.22 m (5H, Ar), 7.32 t (1H, Ar), 7.42 t (2H, Ar), 7.60 t (1H, Ar), 8.07 d (1H, Ar), 8.36-8.60 m br. (2H, Ar and NH), 8.68 s br. (1H, NH), 8.81 s (1H, Ar). LC-MS [M+1]: calc'd: 526.5; obs'd: 526.6.

### 20. N-[4-(aminosulfonyl)phenyl]-4-[4-[(4-fluorobenzyl)amino]-6-(2,2,2-trifluoroethoxy)-1,3,5-triazin-2-yl]benzamide

1H NMR (DMSO-d6, 90° C., ppm): d=4.64 s br. (2H, CH2), 5.08 q (2H, CH2), 6.96 s (1H, Ar), 7.121 t (2H, Ar), 7.43 t (2H, Ar), 7.85 d (1H, Ar), 7.95 d (1H, Ar), 8.09 m (2H, Ar), 8.44 t (2H, Ar), 8.52 s br. (1H, NH), 10.38 s (1H, NH). LC-MS [M+1]: calc'd: 577.5; obs'd: 577.5.

### 21. 4-{4-[(4-fluorobenzyl)amino]-6-(2,2,2-trifluoroethoxy)-1,3,5-triazin-2-yl}-N-[4-(methylsulfonyl)phenyl]benzamide

1H NMR (DMSO-d6, 90° C., ppm): d=3.15 s (3H, CH3), 4.65 s br. (2H, CH2), 5.08 q (2H, CH2), 7.12 t (2H, Ar), 7.44 t (2H, Ar), 7.92 d (2H, Ar), 7.98-8.18 m (4H, Ar), 8.47 d (2H, Ar), 8.54 s br. (1H, NH), 10.50 s (1H, NH). LC-MS [M+1]: calc'd: 576.6; obs'd: 576.5.

### 22. 4-[4-[(4-fluorobenzyl)amino]-6-(2,2,2-trifluoroethoxy)-1,3,5-triazin-2-yl]-N-(4-fluorophenyl)benzamide

1H NMR (DMSO-d6, 90° C., ppm): d=4.64 s br. (2H, CH2), 5.08 q (2H, CH2), 7.14 q (4H, Ar), 7.44 t (2H, Ar), 7.79 t (2H, Ar), 8.08 d (2H, Ar), 8.45 d (2H, Ar), 8.55 s br. (1H, NH), 10.15 s (1H, NH). LC-MS [M+1]: calc'd: 516.4; obs'd: 516.4.

### 23. 4-[4-[(4-fluorobenzyl)amino]-6-(2,2,2-trifluoroethoxy)-1,3,5-triazin-2-yl]-N-phenylbenzamide

1H NMR (DMSO-d6, 90° C., ppm): d=4.65 s br. (2H, CH2), 5.08 q (2H, CH2), 7.12 t (3H, Ar), 7.26-7.56 m (4H, Ar), 7.77 d (2H, Ar), 8.08 d (2H, Ar), 8.45 d (2H, Ar), 8.54 s br. (1H, NH), 10.07 s (1H, NH). LC-MS [M+1]: calc'd: 498.5; obs'd: 498.6.

### 24. N-[4-(dimethylamino)phenyl]-4-[4-[(4-fluorobenzyl)amino]-6-(2,2,2-trifluoroethoxy)-1,3,5-triazin-2-yl]benzamide

1H NMR (DMSO-d6, 90° C., ppm): d=2.89 s (6H, 2CH3), 4.63 s br. (2H, CH2), 5.06 q (2H, CH2), 6.73 m (2H, Ar), 7.11 t (2H, Ar), 7.43 t (2H, Ar), 7.56 m (2H, Ar), 8.05 m (2H, Ar), 8.41 m (2H, Ar), 8.51 s br. (1H, NH), 9.79 s (1H, NH). LC-MS [M+1]: calc'd: 541.5; obs'd: 541.4.

### 25. (4-{4-[(4-fluorobenzyl)amino]-6-(2,2,2-trifluoroethoxy)-1,3,5-triazin-2-yl}phenyl)(4-propylpiperazin-1-yl)methanone

1H NMR (DMSO-d6, 90° C., ppm): d=0.88 t (3H, CH3), 1.47 m (2H, CH2), 2.31 t (2H, CH2), 2.41 m (4H, 2CH2), 3.49 m (41-1, 2CH2), 4.64 s br. (2H, CH2), 5.06 q (2H, CH2), 7.11 t (2H, Ar), 7.42 t (2H, Ar), 7.50 d (2H, Ar), 8.38 d (2H, Ar), 8.49 s br. (1H, NH). LC-MS [M+1]: calc'd: 533.5; obs'd: 533.4.

### 26. (4-cyclohexyl piperazin-1-yl)(4-{4-[(4-fluorobenzyl)amino]-6-(2,2,2-trifluoroethoxy)-1,3,5-triazin-2-yl}phenyl)methanone

1H NMR (DMSO-d6, 90° C., ppm): d=1.23 m (6H, 3CH2), 1.77 m (4H, 2CH2), 2.30 m (1H, CH), 2.54 m (4H, 2CH2), 2.95 m (4H, 2CH2), 4.64 s br. (2H, CH2), 5.06 q (2H, CH2), 7.11 t (2H, Ar), 7.42 t (2H, Ar), 7.50 d (2H, Ar), 8.38 d (2H, Ar), 8.47 s br. (1H, NH). LC-MS [M+1]: calc'd: 573.6; obs'd: 573.7.

### 27. (4-{4-[(4-fluorobenzyl)amino]-6-(2,2,2-trifluoroethoxy)-1,3,5-triazin-2-yl}phenyl)[4-(furan-2-ylcarbonyl)piperazin-1-yl]methanone

1H NMR (DMSO-d6, 90° C., ppm): d=3.60 m (4H, 2CH2), 3.76 m (4H, 2CH2), 4.64 s br. (2H, CH2), 5.07 q (2H, CH2), 6.59 s (1H, Ar), 6.99 s (1H, Ar), 7.12 t (2H, Ar), 7.42 t (2H, Ar), 7.57 d (2H, Ar), 7.76 s (1H, Ar), 8.40 d (2H, Ar), 8.49 s br. (1H, NH). LC-MS [M+1]: calc'd: 585.5; obs'd: 585.4.

### 28. 3-{4-[(4-{4-[(4-fluorobenzyl)amino]-6-(2,2,2-trifluoroethoxy)-1,3,5-triazin-2-yl}phenyl)carbonyl]piperazin-1-yl}propanenitrile

1H NMR (DMSO-d6, 90° C., ppm): d=2.77 m (6H, 3CH2), 2.92 m (2H, CH2), 3.59 s (4H, 2CH2), 4.62 s br. (2H, CH2), 5.07 q (2H, CH2), 7.12 t (2H, Ar), 7.41 t (2H, Ar), 7.54 d (2H, Ar), 8.39 d (2H, Ar), 8.53 s br. (1H, NH). LC-MS [M+1]: calc'd: 544.5; obs'd: 544.4.

### 29. (4-{4-[(4-fluorobenzyl)amino]-6-(2,2,2-trifluoroethoxy)-1,3,5-triazin-2-yl}phenyl)[4-(propan-2-yl)piperazin-1-yl]methanone

1H NMR (DMSO-d6, 90° C., ppm): d=1.30 d (6H, 2CH3), 3.16 m (1H, CH), 3.27 m (4H, 2CH2), 3.76 m (4H, CH2), 4.62 s br. (2H, CH2), 5.07 q (2H, CH2), 7.12 t (2H, Ar), 7.42 t (2H, Ar), 7.62 d (2H, Ar), 8.41 d (2H, Ar), 8.55 s br. (1H, NH). LC-MS [M+1]: calc'd: 533.5; obs'd: 533.5.

### 30. (4-{4-[(4-fluorobenzyl)amino]-6-(2,2,2-trifluoroethoxy)-1,3,5-triazin-2-yl}phenyl)[4-(2-methylpropyl)piperazin-1-yl]methanone

1H NMR (DMSO-d6, 90° C., ppm): d=0.99 d (6H, 2CH3), 2.07 m (1H, CH), 2.91 d (2H, CH2), 3.16 m (4H, 2CH2), 3.76 m (4H, 2CH2), 4.62 s br. (2H, CH2), 5.06 q (2H, CH2), 7.12 t (2H, Ar), 7.42 t (2H, Ar), 7.59 d (2H, Ar), 8.40 d (2H, Ar), 8.54 s br. (1H, NH). LC-MS [M+1]: calc'd: 547.6; obs'd: 547.6.

### 31. N-(4-fluorobenzyl)-4-(4-{[4-(methylsulfonyl)piperazin-1-yl]carbonyl}phenyl)-6-(2,2,2-trifluoroethoxy)-1,3,5-triazin-2-amine

1H NMR (DMSO-d6, 90° C., ppm): d=2.50 m (4H, 2CH2), 2.97 s (3H, CH3), 3.20 m (4H, 2CH2), 4.62 s br. (2H, CH2), 5.07 q (2H, CH2), 7.12 t (2H, Ar), 7.42 t (2H, Ar), 7.52 d (1H, Ar), 8.07 s (1H, Ar), 8.32-8.46 m (2H, Ar), 8.54 s br. (1H, NH). LC-MS [M+1]: calc'd: 569.6; obs'd: 569.4.

### 32. 2-{4-[(4-{4-[(4-fluorobenzyl)amino]-6-(2,2,2-trifluoroethoxy)-1,3,5-triazin-2-yl}phenyl)carbonyl]piperazin-1-yl}-1-(piperidin-1-yl)ethanone

1H NMR (DMSO-d6, 90° C., ppm): d=1.50 m (4H, 4CH2), 1.59 m (2H, CH2), 2.53 m (4H, 2CH2), 3.20 s (2H, CH2), 3.36-3.54 m (8H, 4CH2), 4.64 s br. (2H, CH2), 5.06 q (2H, CH2), 7.11 t (2H, Ar), 7.43 t (2H, Ar), 7.51 d (2H, Ar), 8.38 d (2H, Ar), 8.47 s br. (1H, NH). LC-MS [M+1]: calc'd: 616.6; obs'd: 616.5.

### 33. 2-{4-[(4-{4-[(4-fluorobenzyl)amino]-6-(2,2,2-trifluoroethoxy)-1,3,5-triazin-2-yl}phenyl)carbonyl]piperazin-1-yl}-1-(4-methylpiperazin-1-yl)ethanone

1H NMR (DMSO-d6, 90° C., ppm): d=2.21 s (3H, CH3), 2.32 m (4H, 2CH2), 2.53 m (4H, 2CH2), 3.22 s (2H, CH2), 3.50 m (8H, 4CH2), 4.63 s br. (2H, CH2), 5.06 q (2H, CH2), 7.11 t (2H, Ar), 7.42 t (2H, Ar), 7.51 d (2H, Ar), 8.38 d (2H, Ar), 8.47 s br. (1H, NH). LC-MS [M+1]: calc'd: 631.7; obs'd: 631.8.

### 34. 2-{4-[(4-{4-[(4-fluorobenzyl)amino]-6-(2,2,2-trifluoroethoxy)-1,3,5-triazin-2-yl}phenyl)carbonyl]piperazin-1-yl}-1-(morpholin-4-yl)ethanone

1H NMR (DMSO-d6, 90° C., ppm): d=2.54 m (4H, 2CH2), 3.23 s (2H, CH2), 3.50 m (8H, 4CH2), 3.57 m (4H, 2CH2), 4.63 s br. (2H, CH2), 5.06 q (2H, CH2), 7.11 t (2H, Ar), 7.42 t (2H, Ar), 7.51 d (2H, Ar), 8.38 d (2H, Ar), 8.47 s br. (1H, NH). LC-MS [M+1]: calc'd: 618.6; obs'd: 617.7.

### 35. 1-(azepan-1-yl)-2-{4-[(4-{4-[(4-fluorobenzyl)amino]-6-(2,2,2-trifluoroethoxy)-1,3,5-triazin-2-yl}phenyl)carbonyl]piperazin-1-yl}ethanone

1H NMR (DMSO-d6, 90° C., ppm): d=1.53 m (4H, 2CH2), 1.66 m (4H, 2CH2), 2.54 m (4H, 2CH2), 3.21 s (2H, CH2), 3.50 m (8H, 4CH2), 4.64 s br. (2H, CH2), 5.06 q (2H, CH2), 7.11 t (2H, Ar), 7.42 t (2H, Ar), 7.51 d (2H, Ar), 8.38 d (2H, Ar), 8.47 s br. (1H, NH). LC-MS [M+1]: calc'd: 630.7; obs'd: 630.8.

### 36. (4-{4-[(4-fluorobenzyl)amino]-6-(2,2,2-trifluoroethoxy)-1,3,5-triazin-2-yl}phenyl)[4-(thiophen-2-ylsulfonyl)piperazin-1-yl]methanone

1H NMR (DMSO-d6, 90° C., ppm): d=3.10 t (4H, 2CH2), 3.61 m (4H, 2CH2), 4.62 s br. (2H, CH2), 5.06 q (2H, CH2), 7.12 t (2H, Ar), 7.28 m (1H, Ar), 7.41 t (2H, Ar), 7.50 d (2H, Ar), 7.64 m (1H, Ar), 8.02 m (1H, Ar), 8.36 d (2H, Ar), 8.53 s br. (1H, NH). LC-MS [M+1]: calc'd: 637.7; obs'd: 637.6.

### 37. (3-{4-[(4-fluorobenzyl)amino]-6-(2,2,2-trifluoroethoxy)-1,3,5-triazin-2-yl}phenyl)(4-propylpiperazin-1-yl)methanone

1H NMR (DMSO-d6, 90° C., ppm): d=0.88 t (3H, CH3), 1.46 m (2H, CH2), 2.30 t (2H, CH2), 2.41 m (4H, 2CH2), 3.50 m (4H, 2CH2), 4.63 s br. (2H, CH2), 5.06 q (2H, CH2), 7.11 t (2H, Ar), 7.41 t (2H, Ar), 7.59 d (2H, Ar), 8.31 s (1H, Ar), 8.39 t (1H, Ar), 8.48 s br. (1H, NH). LC-MS [M+1]: calc'd: 533.5; obs'd: 533.5.

### 38. ethyl 4-[(3-{4-[(4-fluorobenzyl)amino]-6-(2,2,2-trifluoroethoxy)-1,3,5-triazin-2-yl}phenyl)carbonyl]piperazine-1-carboxylate

1H NMR (DMSO-d6, 90° C., ppm): d=1.21 t (3H, CH3), 3.46 m (4H, 2CH2), 3.52 m (4H, 2CH2), 4.10 q (2H, CH2), 4.63 s br. (2H, CH2), 5.06 q (2H, CH2), 7.11 t (2H, Ar), 7.41 t (2H, Ar), 7.53-7.72 m (2H, Ar), 8.34 s (1H, Ar), 8.40 d (1H, Ar), 8.48 s br. (1H, NH). LC-MS [M+1]: calc'd: 563.5; obs'd: 563.5.

### 39. (3-{4-[(4-fluorobenzyl)amino]-6-(2,2,2-trifluoroethoxy)-1,3,5-triazin-2-yl}phenyl)[4-(furan-2-ylcarbonyl)piperazin-1-yl]methanone

1H NMR (DMSO-d6, 90° C., ppm): d=3.61 m (4H, 2CH2), 3.75 m (4H, 2CH2), 4.63 s br. (2H, CH2), 5.06 q (2H, CH2), 6.59 t (1H, Ar), 6.99 d (1H, Ar), 7.11 t (2H, Ar), 7.41 t (2H, Ar), 7.52-7.70 m (2H, Ar), 7.75 d (1H, Ar), 8.37 s (1H, Ar), 8.41 d (1H, Ar), 8.51 s br. (1H, NH). LC-MS [M+1]: calc'd: 585.5; obs'd: 585.5.

### 40. (3-{4-[(4-fluorobenzyl)amino]-6-(2,2,2-trifluoroethoxy)-1,3,5-triazin-2-yl}phenyl)[4-(2-methylpropyl)piperazin-1-yl]methanone

1H NMR (DMSO-d6, 90° C., ppm): d=0.88 d (6H, 2CH3), 1.76 s (1H, CH), 2.09 d (2H, CH2), 2.39 m (4H, 2CH2), 3.50 m (4H, 2CH2), 4.61 s br. (2H, CH2), 5.06 q (2H, CH2), 7.11 t (2H, Ar), 7.41 t (2H, Ar), 7.59 d (2H, Ar), 8.31 s (1H, Ar), 8.39 d (1H, Ar), 8.56 s br. (1H, NH). LC-MS [M+1]: calc'd: 547.6; obs'd: 547.7.

### 41. (3-{4-[(4-fluorobenzyl)amino]-6-(2,2,2-trifluoroethoxy)-1,3,5-triazin-2-yl}phenyl)[4-(methylsulfonyl)piperazin-1-yl]methanone

1H NMR (DMSO-d6, 90° C., ppm): d=2.89 s (3H, CH3), 3.22 m (4H, 2CH2), 3.62 m (4H, 2CH2), 4.62 s br. (2H, CH2), 5.07 q (2H, CH2), 7.12 t (2H, Ar), 7.41 t (2H, Ar), 7.55-7.70 m (2H, Ar), 8.35 s (1H, Ar), 8.41 d (1H, Ar), 8.55 s br. (1H, NH). LC-MS [M+1]: calc'd: 569.6; obs'd: 569.3.

### 42. 2-{4-[(3-{4-[(4-fluorobenzyl)amino]-6-(2,2,2-trifluoroethoxy)-1,3,5-triazin-2-yl}phenyl)carbonyl]piperazin-1-yl}-N-(4-methylphenyl)acetamide

1H NMR (DMSO-d6, 90° C., ppm): d=2.27 s (3H, CH3), 2.62 m (4H, 2CH2), 3.17 s (2H, CH2), 3.60 m (4H, 2CH2), 4.63 s br. (2H, CH2), 5.06 q (2H, CH2), 7.03-7.16 m (4H, Ar), 7.36-7.51 m (4H, Ar), 7.60 d (2H, Ar), 8.33 s (1H, Ar), 8.39 t (1H, Ar), 8.47 s br. (1H, NH), 9.35 s br. (1H, NH). LC-MS [M+1]: calc'd: 638.6; obs'd: 638.2.

### 43. 2-{4-[(3-{4-[(4-fluorobenzyl)amino]-6-(2,2,2-trifluoroethoxy)-1,3,5-triazin-2-yl}phenyl)carbonyl]piperazin-1-yl}-1-(piperidin-1-yl)ethanone

1H NMR (DMSO-d6, 90° C., ppm): d=1.52 m (4H, 2CH2), 1.61 m (2H, CH2), 3.00 m (4H, 2CH2), 3.43 m (4H, 2CH2), 3.71 m (4H, 2CH2), 3.81 s (2H, CH2), 4.64 s br. (2H, CH2), 5.06 q (2H, CH2), 7.11 t (2H, Ar), 7.42 t (2H, Ar), 7.53-7.70 m (2H, Ar), 8.35 s (1H, Ar), 8.41 d (1H, Ar), 8.51 s br. (1H, NH). LC-MS [M+1]: calc'd: 616.6; obs'd: 616.6.

### 44. 2-{4-[(3-{4-[(4-fluorobenzyl)amino]-6-(2,2,2-trifluoroethoxy)-1,3,5-triazin-2-yl}phenyl)carbonyl]piperazin-1-yl}-1-(pyrrolidin-1-yl)ethanone

1H NMR (DMSO-d6, 90° C., ppm): d=1.83 m (4H, 2CH2), 2.56 m (4H, 2CH2), 3.16 s (2H, CH2), 3.21-3.59 m (8H, 4CH2), 4.63 s br. (2H, CH2), 5.06 q (2H, CH2), 7.11 t (2H, Ar), 7.42 t (2H, Ar), 7.59 d (2H, Ar), 8.31 s (1H, Ar), 8.39 d (1H, Ar), 8.49 s br. (1H, NH). LC-MS [M+1]: calc'd: 602.6; obs'd: 602.7.

### 45. 2-{4-[(3-{4-[(4-fluorobenzyl)amino]-6-(2,2,2-trifluoroethoxy)-1,3,5-triazin-2-yl}phenyl)carbonyl]piperazin-1-yl}-N-(3-methylphenyl)acetamide

1H NMR (DMSO-d6, 90° C., ppm): d=2.29 s (3H, CH3), 2.62 m (4H, 2CH2), 3.18 s (2H, CH2), 3.59 m (4H, 2CH2), 4.61 s br. (2H, CH2), 5.07 q (2H, CH2), 6.88 s (1H, Ar), 7.03-7.21 m (3H, Ar), 7.33-7.48 m (4H, Ar), 7.54-7.66 m (2H, Ar), 8.32 s (1H, Ar), 8.39 t (1H, Ar), 8.58 s br. (1H, NH), 9.41 s br. (1H, NH). LC-MS [M+1]: calc'd: 638.6; obs'd: 638.5.

### 46. 4-(4-(4-fluorobenzylamino)-6-(2,2,2-trifluoroethoxy)-1,3,5-triazin-2-yl)-N-(2-methylbenzyl)benzamide

1H NMR (DMSO-d6, 90° C., ppm): d=2.32 s (3H, CH3), 4.5 d (2H, CH2), 4.65 br. (2H, CH2), 5.08 q (2H, CH2), 7.05-7.21 m (5H, Ar), 7.31 t (1H, Ar), 7.44 t (2H, Ar), 7.98 d (2H, Ar), 8.40 d (2H, Ar), 8.51 s br. (1H, NH), 8.66 m (1H, NH). LC-MS [M+1]: calc'd: 526.5; obs'd: 526.5.

### Preparation of C(Ar),N,N-Triazines in Table 18

### A. Synthesis of Intermediates and Final Compounds

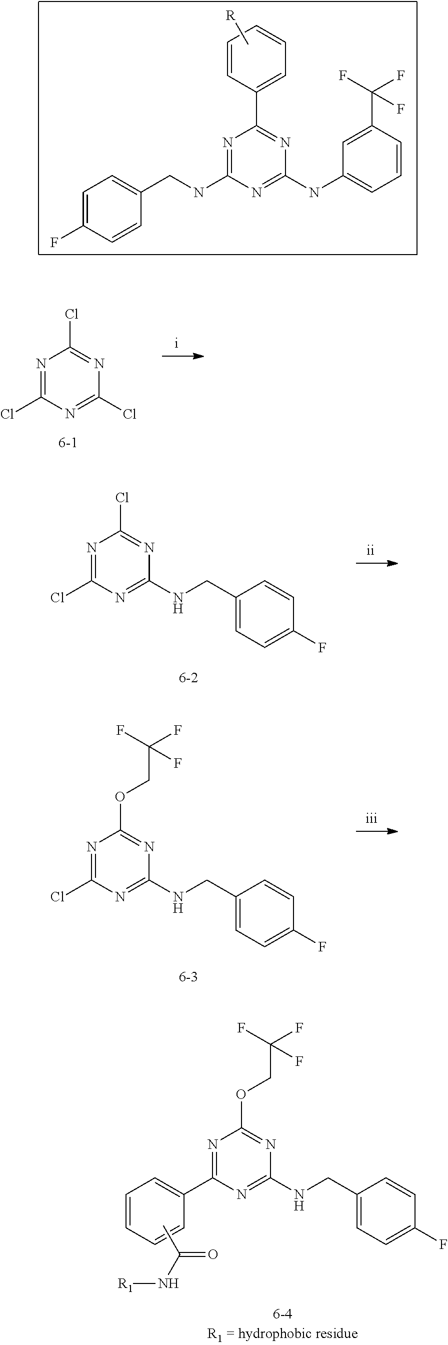

Synthesis of 6-2: In a 1-L flask, 50.0 g (0.27 mol) of cyanuric chloride 6-1 and 24.4 g of sodium acetate were dissolved in 500 mL of dry dioxane. The mixture was cooled to 0 and 34.0 g of 4-fluorobezylamine in 100 mL of dioxane were added within 30 min under constant stirring. The temperature of the reaction mixture was maintained at 0 □C for 2.5 h till the completion of the reaction (TLC control). The solvent was removed in vacuo. The solids were suspended in CHCl₃, and filtered. The filtrate was concentrated in vacuo, and the precipitate was additionally washed with hexane. Yield: 62.2 g, 0.23 mol, 84%. ¹H NMR (DMSO-d₆, 27 ppm): d=4.51 d (2H, CH₂), 7.07 t (2H, Ar), 7.34 t (2H, Ar), 9.58 t br. (1H, NH). LC-MS: Not informative due to the lack of ionization.

Synthesis of 6-3: In a 2-L flask, 35.0 g (0.128 mol) of 6-2 were dissolved in 1 L of acetonitrile. The solution was cooled to −30° C. A solution of potassium tert-butoxide (14.4 g, 0.128 mol, 1 eq.) in 2,2,2-trifluoro-1-ethanol (64 g, 0.64 mol, 5 eq.) was added dropwise to the cooled solution of 6-2 during 1.5 h. After complete addition, the reaction mixture was allowed to warm to r.t., and reaction was allowed to proceed overnight. The solid precipitate was filtered and washed with dry acetonitrile (3×200 mL). The filtrate was concentrated in vacuo producing a yellowish oil. To this oil, dry hexane (3×80 mL) was added, the mixture was heated to reflux, and in 3 min the hexane layer was decanted. After three washes, the residue was first dried using rotary evaporation, followed by lyophilization. Yield: 32.2 g, 0.10 mol, 75%. ¹H NMR (DMSO-d₆, 90° C., ppm): d=4.53 s (2H, CH₂), 4.95 q (2H, CH₂), 7.10 t (2H, Ar), 7.36 t (2H, Ar), 8.92 s br. (1H, NH). LC-MS: Not informative due to the lack of ionization.

Synthesis of 6-4: Monochlorotriazine 6-3 (0.19 mmol) was dissolved in 1,4-dioxane (5 ml). Boronic acid (0.2 mmol), Na₂CO₃ (5% water solution, 0.5 ml), PdCl₂ (5 mol %) were then added. The reaction mixture was stirred for 12 hours at 80° C. LCMS analysis of the reaction mixture after this time demonstrated presence of product (90%). The reaction mixture was diluted with water (200 ml) and extracted with ethyl acetate. The solvent was removed under reduced pressure to afford final compounds.

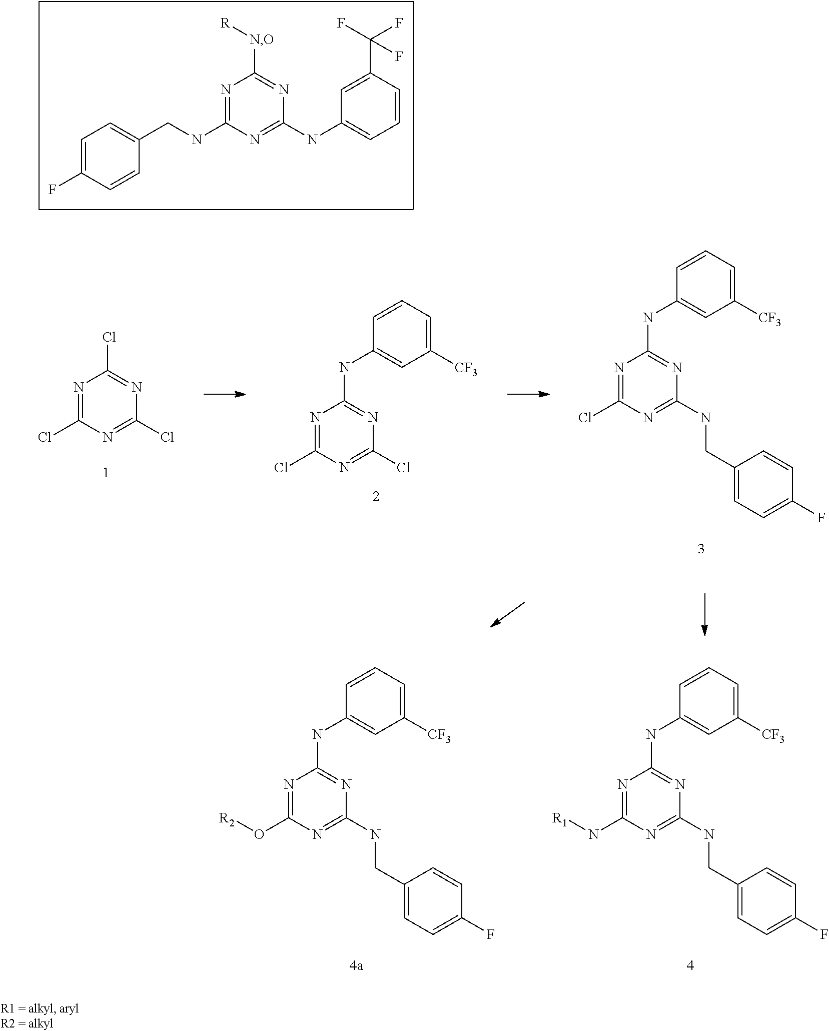

Preparation of 2-(3-trifluoromethylfenylamino)-4-(4-fluorobenzylamino)-6-aminoalkyl-1,3,5-triazines (4) 2-(3-trifluoromethylfenylamino)-4-(4-fluorobenzylamino)-6-chloro-1,3,5-triazine (3) (0.100 g, 0.252 mmol) was suspended in dioxane (5 ml) with K₂CO₃ (0.070 g, 0.504 mmol). The mixture was then treated with amine (0.252 mmol) at ambient temperature. The reaction mixture was stirred for 3-4 hours at 70-80° C. and analyzed by LCMS. The reaction mixture was diluted with water (200 ml) and extracted with ethyl acetate. The solvent was removed under reduced pressure, the precipitate filtered off, and washed with ether to afford the desired product.

Preparation of 2-(3-trifluoromethylfenylamino)-4-(4-fluorobenzylamino)-6-alkoxy-1,3,5-tri-azines (4a) Alcohol (0.300 mmol) was dissolved in dry dioxane (5 ml) and treated with potassium tert-butoxide (0.041 g, 0.360 mmol). The mixture was stirred for 2 h at 60° C. and then cooled to room temperature. 2-Chloro-4-ethoxy-6-(N-3-trifluoromethylanilino)-1,3,5-triazine (0.12 g, 0.300 mmol) was added to this solution and the reaction mixture was heated to 60 The The reaction mixture was stirred at this temperature overnight. The reaction mixture was diluted with water (200 ml) and extracted with ethyl acetate. The solvent was removed under reduced pressure, the precipitate filtered off, and washed with ether to afford the desired product.

TABLE 18

| En | Structure | IUPAC Name | MW | Formula |
|---|---|---|---|---|
| 1 | 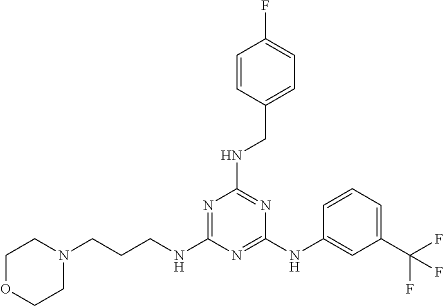 | N-2-(4-fluorobenzyl)-N-4-[3- (morpholin-4-yl)propyl]-N-6-[3- (trifluoromethyl)phenyl]-1,3,5- triazine-2,4,6-triamine | 505.5 | C24H27F4N7O |
| 2 | 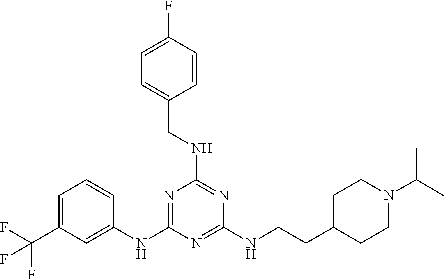 | N-2-(4-fluorobenzyl)-N-4-{2-[1- (propan-2-yl)piperidin-4-yl]ethyl}-N- 6-[3-(trifluoromethyl)-phenyl]-1,3,5- triazine-2,4,6-triamine | 531.6 | C27H33F4N7 |
| 3 | 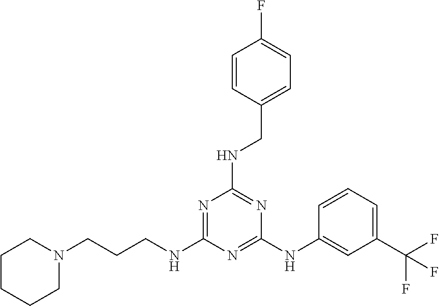 | N-2-(4-fluorobenzyl)-N-4-[3- (piperidin-1-yl)propyl]-N-6-[3- (trifluoromethyl)phenyl]-1,3,5- triazine-2,4,6-triamine | 503.5 | C25H29F4N7 |
| 4 | 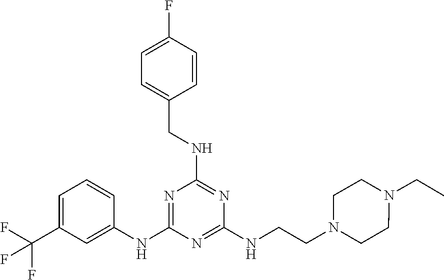 | N-2-[2-(4-ethylpiperazin-1-yl)ethyl]- N-4-(4-fluorobenzyl)-N-6-[3- (trifluoromethyl)phenyl]-1,3,5- triazine-2,4,6-triamine | 518.6 | C25H30F4N8 |
| 5 | 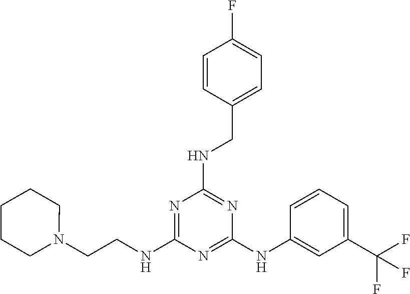 | N-2-(4-fluorobenzyl)-N-4-[2- (piperidin-1-yl)ethyl]-N-6-[3- (trifluoromethyl)phenyl]-1,3,5- triazine-2,4,6-triamine | 489.5 | C24H27F4N7 |
| 6 | 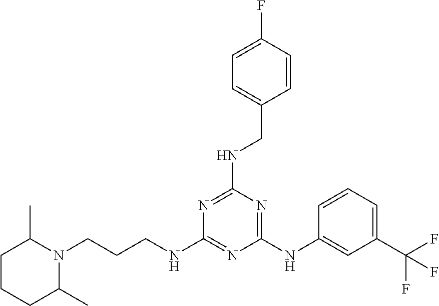 | N-2-[3-(2,6-dimethylpiperidin-1- yl)propyl]-N-4-(4-fluorobenzyl)-N-6- [3-(trifluoromethyl)phenyl]-1,3,5- triazine-2,4,6-triamine | 531.6 | C27H33F4N7 |
| 7 | 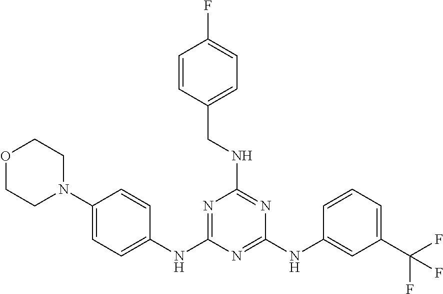 | N-2-(4-fluorobenzyl)-N-4-[4- (morpholin-4-yl)phenyl]-N-6-[3- (trifluoromethyl)phenyl]-1,3,5- triazine-2,4,6-triamine | 539.5 | C27H25F4N7O |
| 8 | 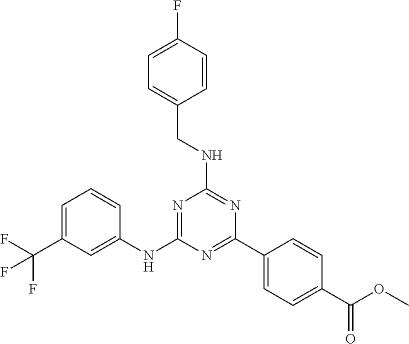 | methyl 4-(4-[(4-fluorobenzyl)amino]- 6-{[3-(trifluoromethyl)phenyl]amino}- 1,3,5-triazin-2-yl)benzoate | 497.4 | C25H19F4N5O2 |
| 9 | 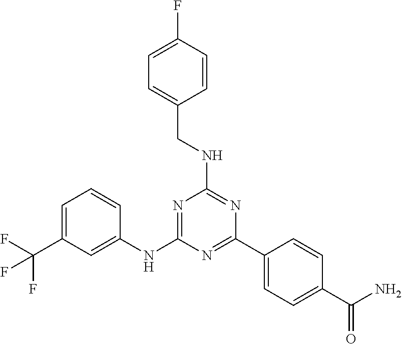 | 4-(4-[(4-fluorobenzyl)amino]-6-{[3- (trifluoromethyl)phenyl]amino}-1,3,5- triazin-2-yl)benzamide | 482.4 | C24H18F4N6O |
| 10 | 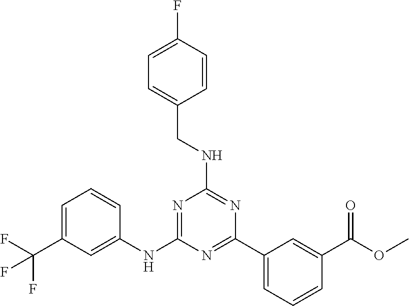 | methyl 3-(4-[(4-fluorobenzyl)amino]- 6-{[3-(trifluoromethyl)phenyl]amino}- 1,3,5-triazin-2-yl)benzoate | 497.4 | C25H19F4N5O2 |
| 11 | 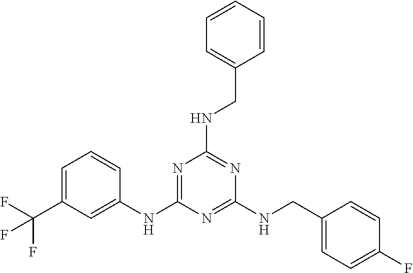 | N-2-benzyl-N-4-(4-fluorobenzyl)-N- 6-[3-(trifluoromethyl)phenyl]-1,3,5- triazine-2,4,6-triamine | 468.5 | C24H20F4N6 |
| 12 | 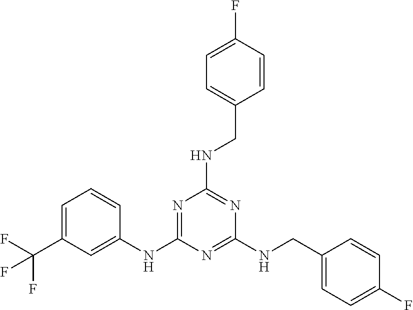 | N-2-,N-4-bis(4-fluorobenzyl)-N-6-[3- (trifluoromethyl)phenyl]-1,3,5- triazine-2,4,6-triamine | 486.4 | C24H19F5N6 |
| 13 | 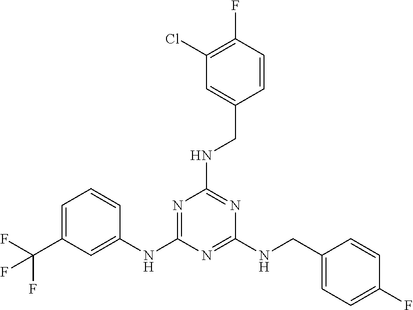 | N-2-(3-chloro-4-fluorobenzyl)-N-4- (4-fluorobenzyl)-N-6-[3- (trifluoromethyl)phenyl]-1,3,5- triazine-2,4,6-triamine | 520.9 | C24H18ClF5N6 |
| 14 | 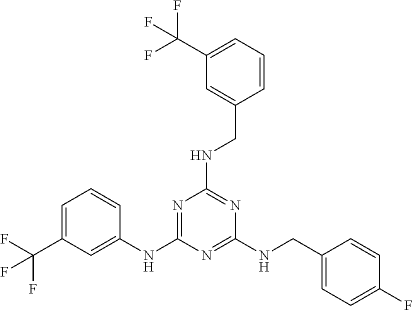 | N-2-(4-fluorobenzyl)-N-4-[3- (trifluoromethyl)-benzyl]-N-6-[3- (trifluoromethyl)phenyl]-1,3,5- triazine-2,4,6-triamine | 536.4 | C25H19F7N6 |
| 15 | 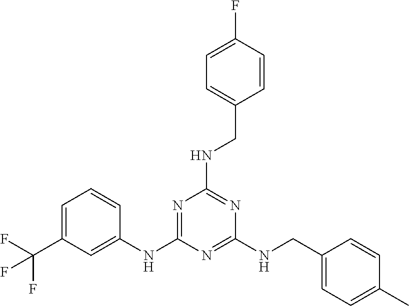 | N-2-(4-fluorobenzyl)-N-4-(4- methylbenzyl)-N-6-[3- (trifluoromethyl)phenyl]-1,3,5- triazine-2,4,6-triamine | 482.5 | C25H22F4N6 |
| 16 | 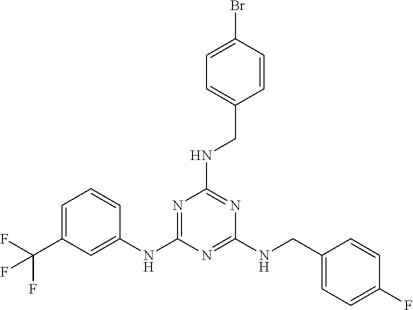 | N-2-(4-bromobenzyl)-N-4-(4- fluorobenzyl)-N-6-[3- (trifluoromethyl)phenyl]-1,3,5- triazine-2,4,6-triamine | 547.3 | C24H19BrF4N6 |
| 17 | 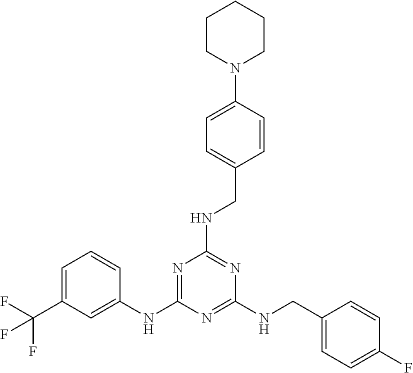 | N-2-(4-fluorobenzyl)-N-4-[4- (piperidin-1-yl)benzyl]-N-6-[3- (trifluoromethyl)phenyl]-1,3,5- triazine-2,4,6-triamine | 551.6 | C29H29F4N7 |
| 18 | 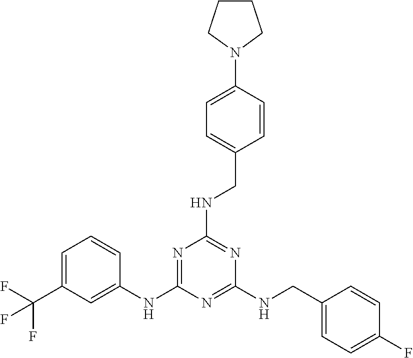 | N-2-(4-fluorobenzyl)-N-4-[4- (pyrrolidin-1-yl)benzyl]-N-6-[3- (trifluoromethyl)phenyl]-1,3,5- triazine-2,4,6-triamine | 537.6 | C28H27F4N7 |
| 19 | 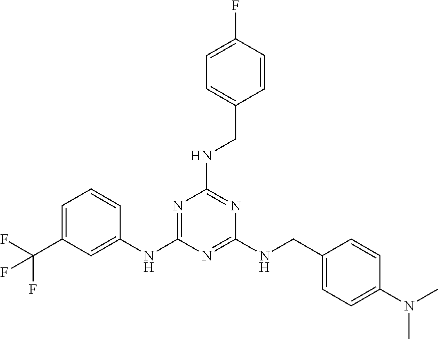 | N-2-[4-(dimethylamino)benzyl]-N-4- (4-fluorobenzyl)-N-6-[3- (trifluoromethyl)phenyl]-1,3,5- triazine-2,4,6-triamine | 511.5 | C26H25F4N7 |
| 20 | 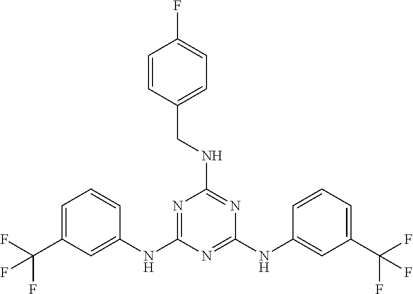 | N-2-(4-fluorobenzyl)-N-4-,N-6-bis[3- (trifluoromethyl)phenyl]-1,3,5- triazine-2,4,6-triamine | 522.4 | C24H17F7N6 |

### B. Procedures and Analytical Data

### 1. N-2-(4-fluorobenzyl)-N-4-[3-(morpholin-4-yl)propyl]-N-6-[3-(trifluoromethyl)phenyl]-1,3,5-triazine-2,4,6-triamine

LCMS: M+1=506.5 NMR 1H, DMSO-d6 d, ppm: 1.40 m (2H); 2.35 m (6H); 3.35 m (2H); 3.60 m (4H); 4.50 m (2H); 6.55 t (1H); 6.95 t (1H); 7.10 t (1H); 7.20 d (2H); 7.40 m (3H); 7.95 d (1H); 8.20 s (1H); 9.80 s (1H).

### 2. N-2-(4-fluorobenzyl)-N-4-{24′-(propan-2-yl)piperidin-4-yl)ethyl}-N-6-[3-(trifluoromethyl)phenyl]-1,3,5-triazine-2,4,6-triamine

LCMS: M+1=532.4 NMR 1H, DMSO-d6 d, ppm: 1.30 m (6H); 1.40-1.70 m (5H); 1.90 m (2H); 2.80 m (1H); 3.35 m (5H); 4.50 d (2H); 6.50 t (1H); 6.90-7.10 m (3H); 7.20 d (1H); 7.30-7.50 m (3H); 7.95 d (1H); 8.40 s (1H); 8.85 s (1H).

### 3. N-2-(4-fluorobenzyl)-N-4-[3-(piperidin-1-yl)propyl]-N-6-[3-(trifluoromethyl)phenyl]-1,3,5-triazine-2,4,6-triamine

LCMS: M+=504.3 NMR 1H, DMSO-d6 δ, ppm: 1.40-1.60 m (6H); 1.70 m (2H); 2.40 m (5H); 3.30 m (2H); 4.50 m (2H); 6.65 s (1H); 7.10 m (3H); 7.20 t (1H); 7.40 m (3H); 7.98 s (1H); 8.25 s (1H); 8.90 s (1H).

### 4. N-2-[2-(4-ethylpiperazin-1-yl)ethyl]-N-4-(4-fluorobenzyl)-N-6-[3-(trifluoromethyl)phenyl]-1,3,5-triazine-2,4,6-triamine

LCMS: M+1=519.5 NMR 1H, DMSO-d6 δ, ppm: 1.10 t (3H); 2.25-2.60 m (12H); 3.40 m (2H); 4.50 m (2H); 6.25 t (1H); 6.90-7.10 m (3H); 7.20 d (1H); 7.30-7.50 m (3H); 7.95 d (1H); 8.20 s (1H); 8.60 s (1H).

### 5. N-2-(4-fluorobenzyl)-N-4-[2-(piperidin-1-yl)ethyl]-N-6-[3-(trifluoromethyl)phenyl]-1,3,5-triazine-2,4,6-triamine

LCMS: M+=490.7 NMR 1H, DMSO-d6 δ, ppm: 1.25-1.65 m (8H); 2.40 m (4H); 3.40 m (2H); 4.50 m (2H); 6.35 s (1H); 7.10 t (1H); 7.20 d (1H); 7.35 m (3H); 7.95 d (1H); 8.20 s (1H); 8.95 s (1H).

### 6. N-2-(4-fluorobenzyl)-N-4-(2-piperidin-1-ylethyl)-N-6-[3-(trifluoromethyl)phenyl]-1,3,5-triazine-2,4,6-triamine

LCMS: M+=490.7 NMR 1H, DMSO-d6 δ, ppm: 1.25-1.65 m (8H); 2.40 m (4H); 3.40 m (2H); 4.50 m (2H); 6.35 s (1H); 7.10 t (1H); 7.20 d (1H); 7.35 m (3H); 7.95 d (1H); 8.20 s (1H); 8.95 s (1H).

### 7. N-2-(4-fluorobenzyl)-N-4-[4-(morpholin-4-yl)phenyl]-N-6-[3-(trifluoromethyl)phenyl]-1,3,5-triazine-2,4,6-triamine

LCMS: M+1=540.6 NMR 1H, DMSO-d6 δ, ppm: 3.10 m (4H); 3.75 m (4H); 4.50 d (2H); 6.86 m (2H); 7.10 t (2H); 7.24 d (1H); 7.32-7.56 m (6H); 7.96-8.10 m (2H); 8.60 (1H); 9.10 s (1H).

### 8. methyl 4-(4-[(4-fluorobenzyl)amino]-6-{[3-(trifluoromethyl)phenyl]amino}-1,3,5-triazin-2-yl)benzoate

1H NMR (DMSO-d6, 90° C., ppm): d=3.92 s (3H, CH3), 4.65 d (2H, CH2), 7.10 t (2H, 2CH), 7.32 d (1H, CH), 7.43 t (2H, 2CH), 7.52 t (1H, CH), 8.00 s (2H, 2CH), 8.08 d (2H, 2CH), 8.31 s (1H, NH), 8.43 d (2H, 2CH), 9.66 s (1H, NH). LC-MS [M+1]: calc'd: 497.4; obs'd: 498.5.

### 9. 4-(4-[(4-fluorobenzyl)amino]-6-{[3-(trifluoromethyl)phenyl]amino}-1,3,5-triazin-2-yl)benzamide

1H NMR (DMSO-d6, 90° C., ppm): d=4.65 d (2H, CH2), 7.32 d (2H, 2CH), 7.42 m (6H, 6CH), 7.98 d (4H, 4CH), 8.36 d (3H, 3CH), 9.62 s (1H, NH). LC-MS [M+1]: calc'd: 482.3; obs'd: 483.4.

### 10. methyl 3-(4-[(4-fluorobenzyl)amino]-6-{[3-(trifluoromethyl)phenyl]amino}-1,3,5-triazin-2-yl)benzoate

1H NMR (DMSO-d6, 90° C., ppm): d=3.92 s (3H, CH3), 4.64 d (2H, CH2), 7.11 t (2H, 2CH), 7.32 d (1H, CH), 7.43 t (2H, 2CH), 7.52 t (1H, CH), 7.66 t (1H, CH), 8.04 (2H, 2CH), 8.13 d (1H, CH), 8.33 s (1H, NH), 8.56 d (1H, CH), 8.95 s (1H, CH), 9.72 s (1H, NH). LC-MS [M+1]: calc'd: 497.4; obs'd: 498.5.

### 11. N-2-benzyl-N-4-(4-fluorobenzyl)-N-6-[3-(trifluoromethyl)phenyl]-1,3,5-triazine-2,4,6-triamine

LCMS: M+1=469.5 NMR 1H, DMSO-d6 δ, ppm: 4.50 m (4H); 6.95-7.12 m (4H); 7.16-7.26 m (2H); 7.24-7.44 m (7H); 7.96 d (1H); 8.17 s (1H); 8.90 s (1H).

### 12. N-2-,N-4-bis(4-fluorobenzyl)-N-6-[3-(trifluoromethyl)phenyl]-1,3,5-triazine-2,4,6-triamine

LCMS: M+=487.5 NMR 1H, DMSO-d6 δ, ppm: 4.50 m (4H); 7.06 m (6H); 7.20 d (1H); 7.28-7.46 m (5H); 7.96 d (1H); 8.16 s (1H); 8.90 s (1H).

### 13. N-2-(3-chloro-4-fluorobenzyl)-N-4-(4-fluorobenzyl)-N-6-[3-(trifluoromethyl)phenyl]-1,3,5-triazine-2,4,6-triamine

LCMS: M+1=521.5 NMR 1H, DMSO-d6 δ, ppm: 4.50 m (4H); 7.00-7.18 m (4H); 7.18-7.50 m (7H); 7.96 d (1H); 8.14 s (1H); 8.90 s (1H).

### 14. N-2-(4-fluorobenzyl)-N-4-[3-(trifluoromethyl)benzyl]-N-6-[3-(trifluoromethyl)phenyl]-1,3,5-triazine-2,4,6-triamine

LCMS: M+1=537.5 NMR 1H, DMSO-d6 δ, ppm: 4.50 d (2H); 4.60 d (2H); 6.98-7.12 m (4H); 7.20 d (1H); 7.28-7.44 m (3H); 7.48-7.58 m (2H); 7.59-7.68 m (2H); 7.95 d (1H); 8.16 s (1H); 8.90 s (1H).

### 15. N-2-(4-fluorobenzyl)-N-4-(4-methylbenzyl)-N-6-[3-(trifluoromethyl)phenyl]-1,3,5-triazine-2,4,6-triamine

LCMS: M+1=483.5 NMR 1H, DMSO-d6 δ, ppm: 2.25 s (3H); 4.50 m (4H); 6.90-7.14 m (6H); 7.20 m (3H); 7.30-7.44 m (3H); 7.97 d (1H); 8.17 s (1H); 8.90 s (1H).

### 16. N-2-(4-bromobenzyl)-N-4-(4-fluorobenzyl)-N-6-[3-(trifluoromethyl)phenyl]-1,3,5-triazine-2,4,6-triamine

LCMS: M+1=547.5 NMR 1H, DMSO-d6 δ, ppm: 4.50 m (4H); 7.00-7.30 m (4H); 7.30-7.50 m (8H); 7.90 d (1H); 8.30 s (1H); 8.80 s (1H); 8.90 s (1H).

### 17. N-2-(4-fluorobenzyl)-N-4-[4-(piperidin-1-yl)benzyl]-N-6-[3-(trifluoromethyl)phenyl]-1,3,5-triazine-2,4,6-triamine

LCMS: M+1=552.4 NMR 1H, DMSO-d6 δ, ppm: 1.60 m (6H); 3.15 m (4H); 4.40 d (2H); 4.50 d (2H); 6.78-6.90 m (3H); 6.94-7.10 m (3H); 7.12-7.24 m (3H); 7.30-7.46 m (3H); 7.95 d (1H); 8.20 s (1H); 8.85 s (1H).

### 18. N-2-(4-fluorobenzyl)-N-4-[4-(pyrrolidin-1-yl)benzyl]-N-6-[3-(trifluoromethyl)phenyl]-1,3,5-triazine-2,4,6-triamine

LCMS: M+1=538.5 NMR 1H, DMSO-d6 δ, ppm: 1.95 m (4H); 3.20 m (4H); 4.40 d (2H); 4.50 d (2H); 6.45 d (2H); 6.80 t (1H); 6.95-7.25 m (6H); 7.30-7.45 m (3H); 8.00 d (1H); 8.35 s (1H); 8.85 s (1H).

### 19. N-2-[4-(dimethylamino)benzyl]-N-4-(4-fluorobenzyl)-N-6-[3-(trifluoromethyl)phenyl]-1,3,5-triazine-2,4,6-triamine

LCMS: M+1=512.7 NMR 1H, DMSO-d6 δ, ppm: 2.85 s (6H); 4.40 d (2H); 4.60 d (2H); 6.65 d (2H); 6.80 t (1H); 6.92-7.22 m (6H); 7.18-7.46 m (3H); 7.95 d (1H); 8.18 (1H); 8.85 s (1H).

### 20. N-2-(4-fluorobenzyl)-N-4-,N-6-bis[3-(trifluoromethyl)phenyl]-1,3,5-triazine-2,4,6-triamine

LCMS: M+1=540.6 NMR 1H, DMSO-d6 δ, ppm: 3.10 m (4H); 3.75 m (4H); 4.50 d (2H); 6.86 m (2H); 7.10 t (2H); 7.24 d (1H); 7.32-7.56 m (6H); 7.96-8.10 m (2H); 8.60 (1H); 9.10 s (1H).

### Section 4. Preparation of R6-Triazine Libraries

### Generic Synthesis of Intermediates and Final Compounds for Table 19.

Compounds of that series were constructed through linear 3 step synthesis according to 4 representative Schemes:

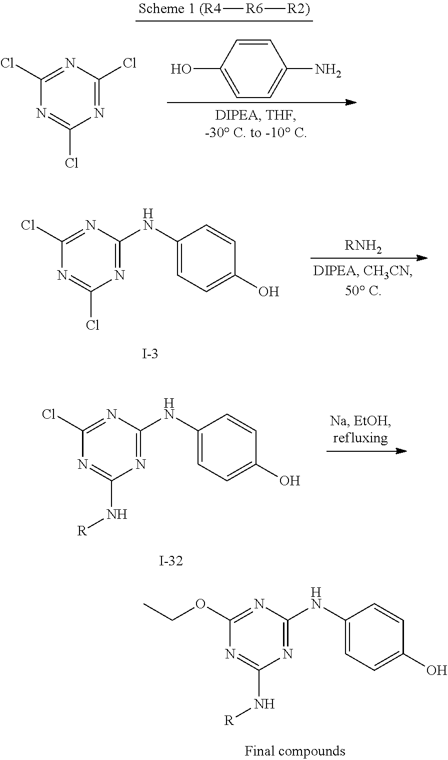

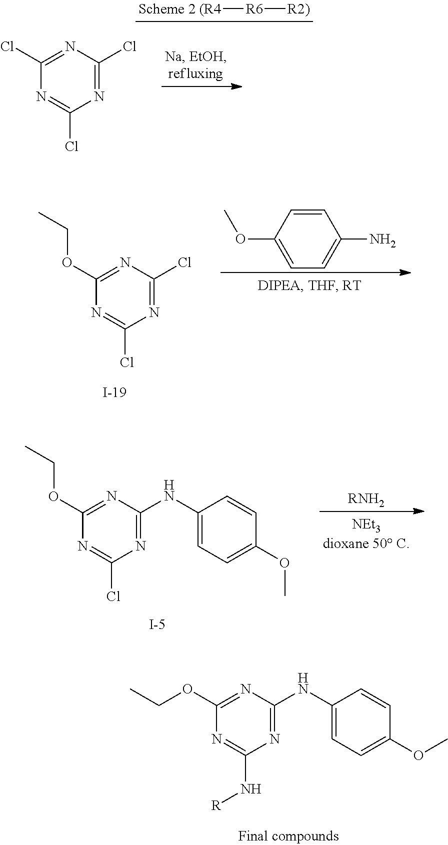

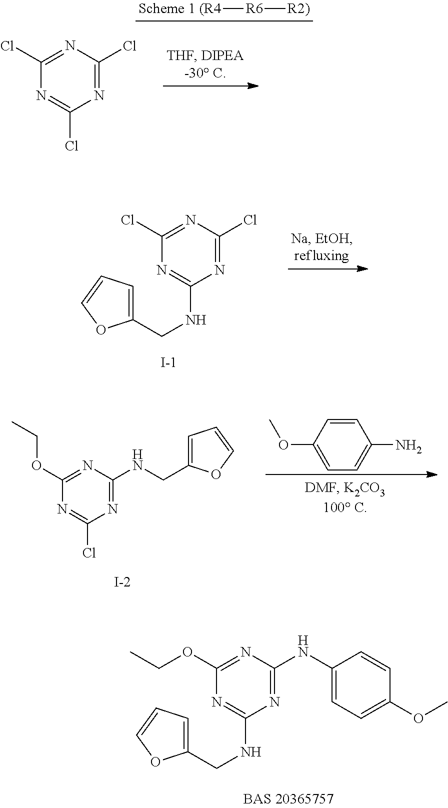

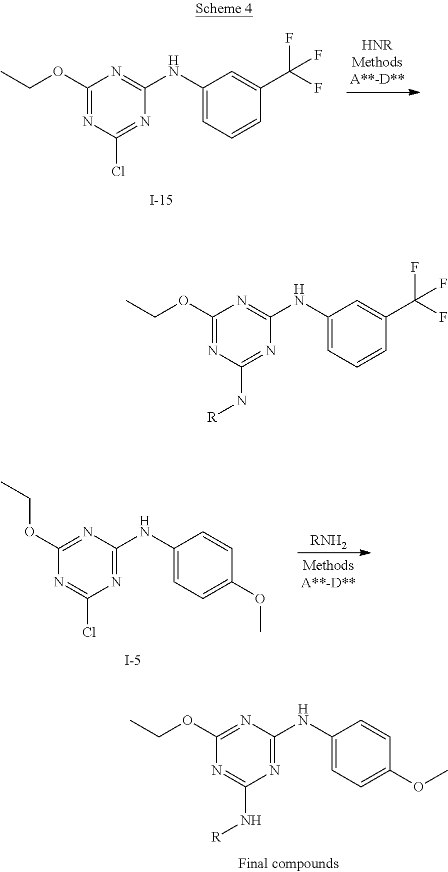

| Method | Conditions | Yields |
|---|---|---|
| A** | DIPEA, THF, RT-100^{° C.}, 2-4 h | 7-50% |
| B** | DIPEA or NEt₃, acetonitrile, RT or 50^{° C.} or | 15-85% |
| | refluxing, 3-12 h | |
| C** | K₂CO₃, DMSO, 60-100^{° C.}, 4 h | 36-57% |
| D** | DIPEA or NEt₃, dioxane, 50^{° C.}, 3-16 h | — |

### Generic Procedure for Method A**

A mixture of I-5 or I-15 (1.78 mmol), amine (1.78 mmol), DIPEA (230 mg, 1.78 mmol) and THF (10 mL) was stirred at room temperature or at 50° C. or at 100° C. for 2-4 hours, cooled to room temperature, concentrated and subjected to preparative TLC to give a final compound.

### Generic Procedure for Method B**

A mixture of I-5 or I-15 (1.0 mmol), corresponding amine (1.1 mmol), DIPEA (0.192 mL, 1.1 mmol) and acetonitrile or NEt₃ (5 mL) was stirred at room temperature or at 50° C. or refluxing for up to 8 hours (TLC control), cooled down to room temperature, diluted with water, filtered or extracted with chloroform. The combined organic phases were concentrated at reduced pressure. Purification by purification by appropriate method furnished a final compound.

Generic Procedure for Method C** A mixture of I-5 or I-15 (1.5 mmol), corresponding amine (1.5 mmol), K₂CO₃ (417 mg, 3.0 mmol) and DMSO (0.2 mL) was stirred at 60-100° C. for 3-16 hours (TLC control), cooled down to room temperature, diluted with water, extracted with dichloromethane. The combined organic phases were concentrated at reduced pressure. Purification by appropriate method furnished a final compound.

TABLE 19

| En | Structure | IUPAC name | MW | Formula |
|---|---|---|---|---|
| 1 | 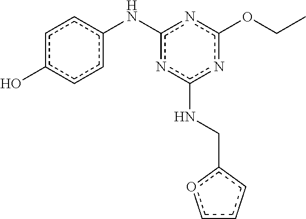 | 4-({4-ethoxy-6-[(furan-2- ylmethyl)amino]-1,3,5-triazin-2- yl}amino)phenol | 327.3 | C16H17N5O3 |
| 2 | 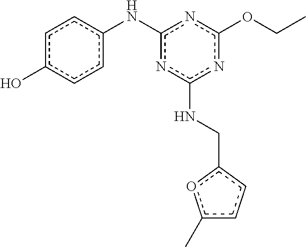 | 4-[(4-ethoxy-6-{[(5-methylfuran-2- yl)methyl]amino}-1,3,5-triazin- 2-yl)amino]phenol | 341.4 | C17H19N5O3 |
| 3 | 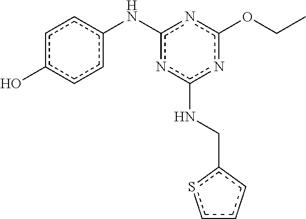 | 4-({4-ethoxy-6-[(thiophen-2- ylmethyl)amino]-1,3,5-triazin-2- yl}amino)phenol | 343.4 | C16H17N5O2S |
| 4 | 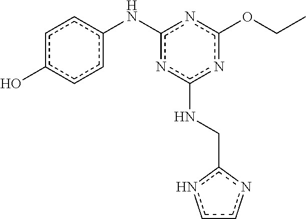 | 4-({4-ethoxy-6-[(1H-imidazol-2- ylmethyl)amino]-1,3,5-triazin-2- yl}amino)phenol | 327.3 | C15H17N7O2 |
| 5 | 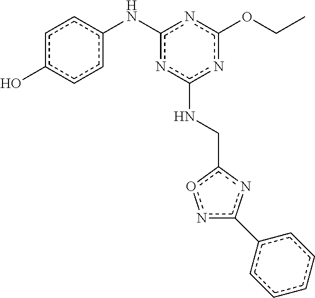 | 4-[(4-ethoxy-6-{[(3-phenyl-1,2,4- oxadiazol-5-yl)methyl]amino}- 1,3,5-triazin-2-yl)amino]phenol | 405.4 | C20H19N7O3 |
| 6 | 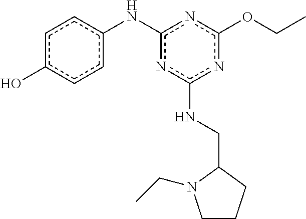 | 4-[(4-ethoxy-6-{[(1-ethylpyrrolidin- 2-yl)methyl]amino}-1,3,5-triazin- 2-yl)amino]phenol | 358.4 | C18H26N6O2 |
| 7 | 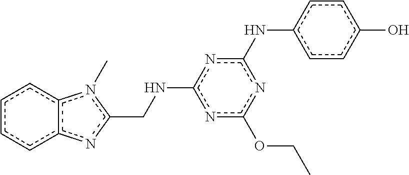 | 4-[(4-ethoxy-6-{[(1-methyl-1H- benzimidazol-2-yl)methyl]amino}- 1,3,5-triazin-2-yl)amino]phenol | 391.4 | C20H21N7O2 |
| 8 | 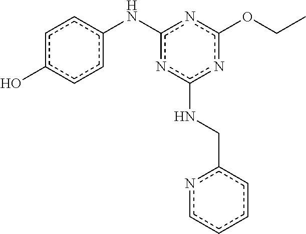 | 4-({4-ethoxy-6-[(pyridin-2- ylmethyl)amino]-1,3,5-triazin-2- yl}amino)phenol | 338.4 | C17H18N6O2 |
| 9 | 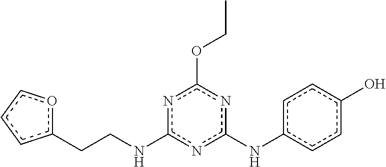 | 4-[(4-ethoxy-6-{[2-(furan-2- yl)ethyl]amino}-1,3,5-triazin-2- yl)amino]phenol | 341.4 | C17H19N5O3 |
| 10 | 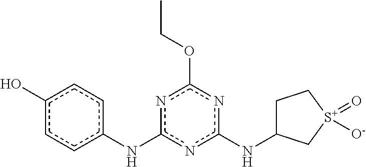 | 4-({4-[(1,1- dioxidotetrahydrothiophen-3- yl)amino]-6-ethoxy-1,3,5- triazin-2-yl}amino)phenol | 365.4 | C15H19N5O4S |
| 11 | 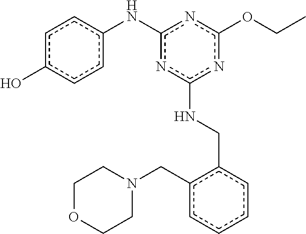 | 4-[(4-ethoxy-6-{[2-(morpholin- 4-ylmethyl)benzyl)amino}- 1,3,5-triazin-2-yl)amino]phenol | 436.5 | C23H28N6O3 |
| 12 | 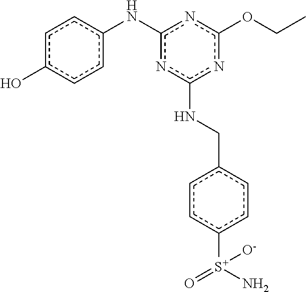 | 4-[({4-ethoxy-6-[(4- hydroxyphenyl)amino]-1,3,5- triazin-2- yl}amino)methyl]benzenesulfon amide | 416.5 | C18H20N6O4S |
| 13 | 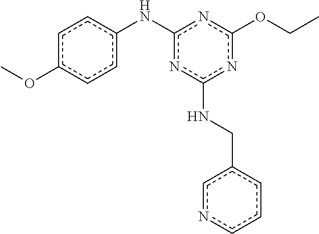 | 6-ethoxy-N-(4-methoxyphenyl)- N′-(pyridin-3-ylmethyl)-1,3,5- triazine-2,4-diamine | 352.4 | C18H20N6O2 |
| 14 | 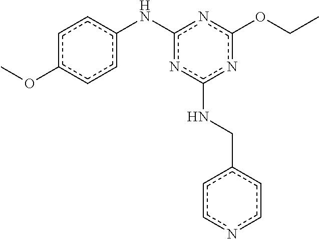 | 6-ethoxy-N-(4-methoxyphenyl)- N′-(pyridin-4-ylmethyl)-1,3,5- triazine-2,4-diamine | 352.4 | C18H20N6O2 |
| 15 | 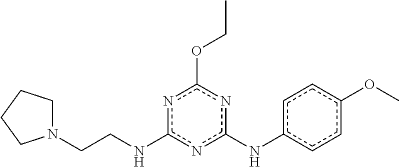 | 6-ethoxy-N-(4-methoxyphenyl )- N′[2-(pyrrolidin-1-yl)ethyl]- 1,3,5-triazine-2,4-diamine | 358.4 | C18H26N6O2 |
| 16 | 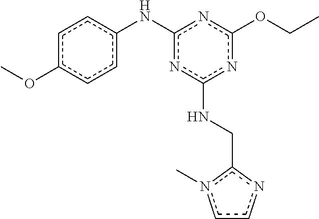 | 6-ethoxy-N-(4-methoxyphenyl)- N′-[(1-methyl-1H-imidazol-2- yl)methyl]-1,3,5-triazine-2,4- diamine | 355.4 | C17H21N7O2 |
| 17 | 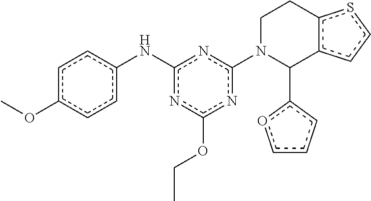 | 4-ethoxy-6-[4-(furan-2-yl)-6,7- dihydro-thieno[3,2-c]pyridin- 5(4H)-yl]-N-(4-methoxyphenyl)- 1,3,5-triazin-2-amine | 449.5 | C23H23N5O3S |
| 18 | 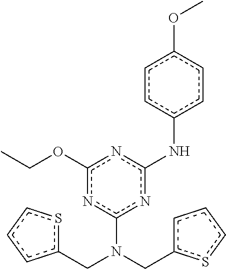 | 6-ethoxy-N′-(4-methoxyphenyl)- N,N-bis(thiophen-2-ylmethyl)- 1,3,5-triazine-2,4-diamine | 453.6 | C22H23N5O2S2 |
| 19 | 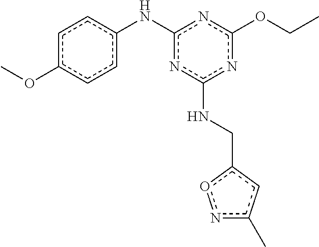 | 6-ethoxy-N-(4-methoxyphenyl)- N′-[(3-methylisoxazol-5-yl)methyl]- 1,3,5-triazine-2,4-diamine | 356.4 | C17H20N6O3 |
| 20 | 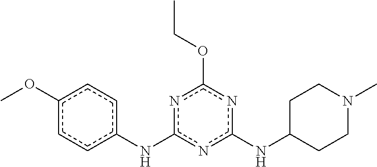 | 6-ethoxy-N-(4-methoxyphenyl )- N′-(1-methylpiperidin-4-yl)- 1,3,5-triazine-2,4-diamine | 358.4 | C18H26N6O2 |
| 21 | 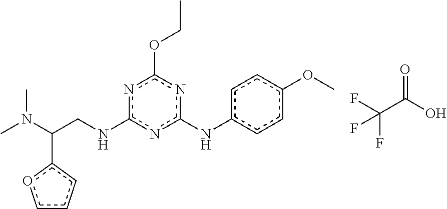 | N-[2-(dimethylamino)-2-(furan-2- yl)ethyl]-6-ethoxy-N′-(4- methoxyphenyl)-1,3,5-triazine- 2,4-diamine trifluoroacetate | 512.5 | C22H27F3N6O5 |
| 22 | 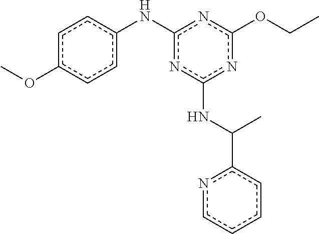 | 6-ethoxy-N-(4-methoxyphenyl)- N′[1-(pyridin-2-yl)ethyl]-1,3,5- triazine-2,4-diamine | 366.4 | C19H22N6O2 |
| 23 | 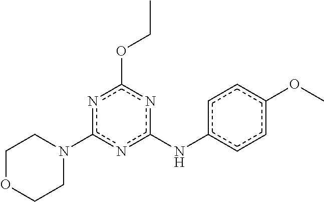 | 4-ethoxy-N-(4-methoxyphenyl )- 6-(morpholin-4-yl)-1,3,5-triazin- 2-amine | 331.4 | C16H21N5O3 |

### Procedures and Analytical Data for Table 19.

### 1. 2-(4-Hydroxy-phenylamino)-4-Ethoxy-6-[(furan-2-ylmethyl)-amino]-[1,3,5]triazine

Sodium (15 mg, 0.63 mmol) was dissolved in ethanol (0.3 mL). The obtained solution was added dropwise at room temperature to a solution of I-45 (200 mg, 0.63 mmol) in ethanol (2 mL). The resulting mixture was stirred at room temperature for 1 hour, then at refluxing for 4 hours (TLC control), cooled down to room temperature, concentrated at reduced pressure. Purification by column chromatography on silica gel (ethyl acetate/hexane) furnished the product (72 mg, 35%). ¹H-NMR (400 MHz, DMSO-D₆) δ_{H}: 1.24 (3H, t, J=7.5 Hz), 4.24 (2H, broad), 4.46 (2H, broad), 6.22 (1H, broad), 6.36 (1H, broad), 6.76 (2H, d, J=8.5 Hz), 7.42 (2H, d, J=8.5 Hz, broad), 7.51 (1H, d, J=1.8 Hz), 7.62 (1H, broad), 8.94 (1H, s), 9.00-9.50 (1H, broad, Z/E forms). LCMS t_{R} (min) 1.50. MS (APCI), m/z 327.70 [M+H]⁺. Mₚ 45° C.

### 2. 4-{4-Ethoxy-6-[(5-methyl-furan-2-ylmethyl)-amino]-[1,3,5]triazin-2-ylamino}-phenol

To a solution of I-3 (514 mg, 2.0 mmol) in THF (10 mL) a solution of 5-methylfurfurylamine (222 mg, 2.0 mmol) and N,N-diisopropylethylamine (260 mg, 2.0 mmol) in THF (10 mL) was added slowly dropwise at 0° C. The resulting mixture was stirred at 0° C. for 2 hours and 3 hours at room temperature (TLC control) and concentrated at reduced pressure. Purification by column chromatography on silica gel (ethyl acetate/hexane) gave mono-chloro-intermediate (463 mg, 70%).

Sodium (46 mg, 2.0 mmol) was dissolved in anhydrous ethanol (1 mL) at room temperature. The obtained solution was added dropwise to a solution of the mono-chloro-intermediate (332 mg, 1.0 mmol) in anhydrous ethanol (3 mL). The resulting mixture was stirred at room temperature for 20 minutes, and then at refluxing for 2 hours. After completion of the reaction (TLC control) the solvent was removed at reduced pressure. The reaction mixture was washed with water (10 mL) and extracted with chloroform (3×5 mL). The combined organic phases were concentrated at reduced pressure. Purification by column chromatography (silica gel, methanol/ethyl acetate) gave a final compound (34 mg, 10%). ¹H-NMR (400 MHz, DMSO-D₆) δ_{H}: 1.23 (3H, t, J=7.5 Hz), 2.19 (3H, s), 4.22 (2H, broad, Z/E forms), 4.36 (2H, d, J=7.5 Hz), 5.91 (1H, broad), 6.05 (1H, broad), 6.63 (2H, d, J=8.5 Hz), 7.41 (2H, d, J=8.5 Hz), 7.54 (1H, broad, Z/E forms), 8.93 (1H, s), 9.15 (1H, broad, Z/E forms). LCMS t_{R} 1.60 (min). MS (APCI), m/z 341.78 [M+H]⁺. Mₚ 40-42° C.

### 3. 4-{4-Ethoxy-6-[(thiophen-2-ylmethyl)-amino]-[1,3,5]triazin-2-ylamino}-phenol

¹H-NMR (400 MHz, DMSO-D₆) δ_{H}: 1.20-1.30 (3H, m, Z/E forms), 4.27 (2H, broad, Z/E forms), 4.61 (2H, d, J=7.5 Hz), 6.73 (2H, d, J=8.5 Hz), 6.92 (1H, dd, J=5.4, 4.0 Hz), 6.97 (1H, d, J=1.5 Hz), 7.30 (1H, d, J=4.0 Hz), 7.41 (2H, d, J=8.5 Hz), 7.68-7.80 (1H, broad, Z/E forms), 8.93 (1H, s), 9.08 (1H, broad, Z/E forms). LCMS t_{R} 1.60 (min). MS (APCI), m/z 343.73 [M+H]⁺. Mₚ 43-45° C.

### 4. 4-{4-Ethoxy-6-[(1H-imidazol-2-ylmethyl)-amino]-[1,3,5]triazin-2-ylamino}-phenol

¹H-NMR (400 MHz, DMSO-D₆) δ_{H}: 1.23 (3H, broad), 4.24 (2H, broad, Z/E forms), 4.53 (2H, broad), 6.65 (2H, d, J=8.5 Hz), 6.97 (2H, d, J=8.5 Hz), 7.00 (1H, s), 7.25-7.60 (2H, broad, Z/E forms), 8.83 (1H, s), 8.95-9.20 (1H, broad, Z/E forms), 12.20 (1H, broad). LCMS t_{R} 1.20 (min). MS (APCI), m/z 327.84 [M+H]⁺. Mₚ 78-80° C.

### 5. 4-{4-Ethoxy-6-[(3-phenyl-[1,2,4]oxadiazol-5-ylmethyl)-amino-]-[1,3,5]triazin-2-ylamino}-phenol

¹H-NMR (400 MHz, DMSO-D₆) δ_{H}: 1.26 (3H, broad), 4.15-4.40 (2H, broad, Z/E forms), 4.80 (2H, broad), 6.64 (2H, d, J=8.5 Hz), 7.20-7.50 (3H, m), 7.55 (2H, d, J=8.5 Hz), 7.57 (1H, s), 8.00 (2H, d, J=8.5 Hz), 8.96 (1H, broad), 9.12 (1H, s). LCMS t_{R} 1.65 (min). MS (APCI), m/z 405.77 [M+H]⁺. Mₚ 105-107° C.

### 6. 4-{4-Ethoxy-6-[(1-ethyl-pyrrolidin-2-ylmethyl)-amino]-[1,3,5]triazin-2-ylamino}-phenol

¹H-NMR (400 MHz, DMSO-D₆) δ_{H}: 1.10-1.30 (6H, m), 1.70-2.20 (4H, m, broad, Z/E forms), 3.05 (2H, broad), 3.30-3.80 (5H, m, broad, Z/E forms), 4.28 (2H, broad, Z/E forms), 6.70 (2H, broad, Z/E forms), 7.40 (2H, broad, Z/E forms), 7.48 (1H, broad, Z/E forms), 9.10 (1H, broad), 10.00-10.35 (1H, broad, Z/E forms). LCMS t_{R} 1.26 (min). MS (APCI), m/z 358.87 [M+H]⁺. Mₚ 96-98° C.

### 7. 4-{4-Ethoxy-6-[(1-methyl-1H-benzoimidazol-2-ylmethyl)-amino]-[1,3,5]triazin-2-ylamino}-phenol

¹H-NMR (400 MHz, DMSO-D₆) δ_{H}: 1.23 (3H, broad, Z/E forms), 3.78 (3H, s), 4.23 (2H, broad, Z/E forms), 4.71 (2H, broad, Z/E forms), 6.62 (2H, broad, Z/E forms), 7.14 (1H, t, J=8.5 Hz), 7.18 (1H, t, J=8.5 Hz), 7.25-7.50 (2H, broad), 7.48 (1H, d, J=8.5 Hz), 7.54 (1H, d, J=8.5 Hz), 7.64 (1H, broad), 8.91 (1H, s), 9.03 (1H, broad). LCMS t_{R} 1.29 (min). MS (APCI), m/z 391.81 [M+H]⁺. Mₚ 119-121° C.

### 8. 4-{4-Ethoxy-6-[(pyridin-2-ylmethyl)-amino]-[1,3,5]triazin-2-ylamino}-phenol

¹H-NMR (400 MHz, DMSO-D₆) δ_{H}: 1.28 (3H, t, J=7.5 Hz), 4.10-4.35 (2H, broad, Z/E forms), 4.55 (2H, broad), 6.58 (1H, broad), 6.67 (1H, broad), 7.22 (1H, m), 7.28 (2H, d, J=7.4 Hz), 7.42 (1H, broad), 7.71 (2H, m, Z/E forms), 8.49 (1H, m, broad), 8.90-9.05 (2H, broad, Z/E forms). LCMS t_{R} 1.23 (min). MS (APCI), m/z 338.74 [M+H]⁺. Mₚ 60-62° C.

### 9. 4-[4-Ethoxy-6-(2-furan-2-yl-ethylamino)-[1,3,5]triazin-2-ylamino]-phenol

¹H-NMR (400 MHz, DMSO-D₆) δ_{H}: 1.23 (3H, t, J=7.5 Hz), 2.85 (2H, t, J=7.5 Hz), 3.50 (2H, t, J=7.5 Hz), 4.23 (2H, q, J=7.5 Hz), 6.22 (1H, broad), 6.34 (1H, broad), 6.65 (2H, d, J=8.5 Hz), 7.25-7.40 (1H, broad, Z/E forms), 7.40-7.55 (2H, broad), 7.51 (1H, d, J=7.5 Hz), 8.85-9.15 (1H, broad, Z/E/forms), 8.94 (1H, s). LCMS t_{R} 2.15 (min). MS (APCI), m/z 342.01 [M+H]⁺. Mₚ 45-47° C.

### 10. 4-[4-(1λ*6*-thiophen-3-ylamino)-6-ethoxy-[1,3,5]triazin-2-ylamino]-phenol

¹H-NMR (400 MHz, DMSO-D₆) δ_{H}: 1.24 (3H, t, J=7.5 Hz), 2.16 (1H, m), 2.97 (1H, m), 3.05-3.50 (4H, m), 4.24 (2H, q, J=7.5 Hz), 4.58 (1H, broad, Z/E forms), 6.65 (2H, d, J=8.5 Hz), 7.39 (2H, d, J=8.5 Hz), 7.57 (1H, broad), 8.97 (1H, broad), 9.15 (1H, broad). LCMS t_{R} 1.39 (min). MS (APCI), m/z 365.80 [M+H]⁺. Mₚ 52-54° C.

### 11. 4-[4-Ethoxy-6-(2-morpholin-4-ylmethyl-benzylamino)[1,3,5]triazin-2-ylamino]-phenol

¹H-NMR (400 MHz, DMSO-D₆) δ_{H}: 1.24 (3H, t, J=7.5 Hz), 2.18 (4H, m), 2.26 (2H, broad), 3.50-3.60 (4H, m), 4.25 (2H, broad), 4.59 (2H, broad), 6.63 (2H, broad), 7.22 (2H, d, J=8.5 Hz), 7.25-8.90 (5H, m, broad), 8.90-9.00 (2H, m, broad, Z/E forms). LCMS t_{R} 1.31 (min). MS (APCI), m/z 436.74 [M+H]⁺. Mₚ 55-57° C.

### 12. 4-{[4-Ethoxy-6-(4-hydroxy-phenylamino)-[1,3,5]triazin-2-ylamino]-methyl}-benzenesulfonamide

¹H-NMR (400 MHz, DMSO-D₆) δ_{H}: 1.23 (3H, broad, Z/E forms), 4.23 (2H, broad, Z/E forms), 4.50 (2H, broad, Z/E forms), 6.62 (2H, broad, Z/E forms), 7.18 (2H, s), 7.40 (3H, broad), 7.45 (2H, d, J=8.5 Hz), 7.74 (2H, d, J=8.5 Hz), 8.90-9.10 (2H, broad, Z/E forms). LCMS t_{R} 1.41 (min). MS (APCI), m/z 416.78 [M+H]⁺. Mₚ 145-147° C.

### 13. 6-Ethoxy-N-(4-methoxy-phenyl)-N′-pyridin-3-ylmethyl-[1,3,5]triazine-2,4-diamine

¹H-NMR (400 MHz, DMSO-D₆) δ_{H}: 1.28 (3H, broad), 3.72 (3H, s), 4.27 (2H, q, J=7.5 Hz), 4.50 (2H, d, J=7.5 Hz), 6.83 (2H, broad), 7.32 (1H, d/d, J=8/5 Hz), 7.45-7.62 (2H, broad, Z/E forms), 7.72 (1H, d, J=8.0 Hz), 7.83 (1H, broad), 8.43 (1H, d, J=5 Hz), 8.55 (1H, d, J=1.5 Hz), 9.08-9.20 (1H, broad, Z/E forms).

### 14. 6-Ethoxy-N-(4-methoxy-phenyl)-N′-pyridin-4-ylmethyl-[1,3,5]triazine-2,4-diamine

¹H-NMR (400 MHz, DMSO-D₆) δ_{H}: 1.16-1.35 (3H, broad, Z/E forms), 3.74 (3H, s), 4.15-4.40 (2H, broad, Z/E forms), 4.50 (2H, broad), 6.70-6.90 (2H, broad, Z/E forms), 7.29 (2H, d, J=5 Hz), 7.30-7.70 (2H, broad, Z/E forms), 7.88 (1H, broad, Z/E forms), 8.49 (2H, d, J=5 Hz), 9.16 (1H, broad). LCMS t_{R} (min): 1.38. MS (APCI), m/z 353.07 [M+H]⁺. HPLC t_{R} (min): 8.30. Mₚ 201-203° C.

### 15. 6-Ethoxy-N-(4-methoxy-phenyl)-N′-(2-pyrrolidin-1-yl-ethyl)-[1,3,5]triazine-2,4-diamine

¹H-NMR (400 MHz, DMSO-D₆) δ_{H}: 1.28 (3H, t, J=7.5 Hz), 1.90 (4H, m), 2.80-3.80 (6H, broad, Z/E forms), 3.59 (2H, m), 3.71 (3H, s), 4.29 (2H, broad), 6.88 (2H, broad), 7.41 (1H, broad), 7.59 (2H, broad), 9.12-9.29 (1H, broad, Z/E forms). LCMS t_{R} (min): 1.38. MS (APCI), m/z 359.01 [M+H]⁺. HPLC t_{R} (min): 8.49. Mₚ 76-78° C.

### 16. 6-Ethoxy-N-(4-methoxy-phenyl)-N′-(1-methyl-1H-imidazol-2-ylmethyl)-[1,3,5]triazine-2,4-diamine

¹H-NMR (400 MHz, DMSO-D₆) δ_{H}: 1.27 (3H, t, J=7.5 Hz), 3.62 (3H, s), 3.70 (3H, s), 4.28 (2H, q, J=7.5 Hz), 4.53 (2H, d, J=7.5 Hz), 6.77 (1H, s), 6.84 (2H, d, J=8.5 Hz), 7.05 (1H, s), 7.45-7.65 (3H, m, broad), 9.10-9.30 (1H, broad, Z/E forms). LCMS t_{R} (min): 1.38. MS (APCI), m/z 356.08 [M+H]⁺. HPLC t_{R} (min): 8.64. Mₚ 57-59° C.

### 17. [4-Ethoxy-6-(4-furan-2-yl-6,7-dihydro-4H-thieno[3,2-c]pyridin-5-yl)-[1,3,5]triazin-2-yl]-(4-methoxy-phenyl)-amine

¹H-NMR (400 MHz, DMSO-D₆) δ_{H}: 1.29 (3H, t, J=7.5 Hz), 2.91 (2H, broad), 3.35 (2H, m), 3.74 (3H, s), 4.33 (2H, q, J=7.5 Hz), 4.88-4.99 (1H, broad, Z/E forms), 6.15 (1H, broad), 6.39 (1H, broad), 6.88 (2H, d, J=8.5 Hz), 6.92 (1H, d, J=5.4 Hz), 7.38 (1H, d, 5.4 Hz), 7.56 (2H, d, J=8.5 Hz), 7.60 (1H, s), 9.34 (1H, broad). LCMS t_{R} (min): 2.12. MS (APCI), m/z 450.03 [M+H]⁺. HPLC t_{R} (min): 16.30. Mₚ 89-91° C.

### 18. 6-Ethoxy-N-(4-methoxy-phenyl)-N′,N′-bis-thiophen-2-ylmethyl-[1,3,5]triazine-2,4-diamine

¹H-NMR (400 MHz, DMSO-D₆) δ_{H}: 1.31 (3H, t, J=7.5 Hz), 3.71 (3H, s), 4.38 (2H, q, J=7.5 Hz), 4.90 (4H, s), 6.83 (2H, d, J=8.5 Hz), 6.96 (2H, dd, J=5.4/4.0 Hz), 7.08 (2H, d, J=4.0 Hz), 7.39 (2H, d, J=5.4 Hz), 7.57 (2H, d, J=8.5 Hz), 9.38 (1H, s). LCMS t_{R} (min): 2.22. MS (APCI), m/z 454.08 [M+H]⁺. HPLC t_{R} (min): 16.53. Mₚ 63-65° C.

### 19. 6-Ethoxy-N-(4-methoxy-phenyl)-N′-(3-methyl-isoxazol-5-ylmethyl)-[1,3,5]triazine-2,4-diamine

¹H-NMR (400 MHz, DMSO-D₆) δ_{H}: 1.29 (3H, t, J=7.5 Hz), 2.19 (3H, s), 3.71 (3H, s), 4.29 (2H, q, J=7.5 Hz), 4.54 (2H, broad), 6.12 (1H, s), 6.81 (2H, d, J=8.5 Hz), 7.54 (2H, broad), 7.81 (1H, broad), 9.10-9.30 (1H, broad, Z/E forms). LCMS t_{R} (min): 1.64. MS (APCI), m/z 357.08 [M+H]⁺. HPLC t_{R} (min): 11.30. Mₚ 59-61° C.

### 20 6-Ethoxy-N-(4-methoxy-phenyl)-N′-(1-methyl-piperidin-4-yl)-[1,3,5]triazine-2,4-diamine

¹H-NMR (400 MHz, DMSO-D₆) δ_{H}: 1.28 (3H, t, J=7.5 Hz), 1.52 (2H, m), 1.81 (2H, broad), 2.00 (2H, m), 2.20 (3H, s), 2.79 (2H, m), 3.70 (1H, m), 3.72 (3H, s), 4.28 (2H, q, J=7.5 Hz), 6.84 (2H, d, J=8.5 Hz), 7.06-7.18 (1H, broad, Z/E forms), 7.60 (2H, d, J=8.5 Hz), 8.91-9.18 (1H, broad, Z/E forms). LCMS t_{R} (min): 1.38. MS (APCI), m/z 359.17 [M+H]⁺. HPLC t_{R} (min): 8.18. Mₚ 42-44° C.

### 21. N-(2-Dimethylamino-2-furan-2-yl-ethyl)-6-ethoxy-N′-(4-methoxy-phenyl)-[1,3,5]triazine-2,4-diamine

¹H-NMR (400 MHz, DMSO-D₆) δ_{H}: 1.29 (3H, broad), 2.50 (3H, s), 3.62 (2H, broad), 3.70 (6H, s), 3.95 (1H, broad), 4.28 (2H, broad), 6.30 (1H, broad), 6.41 (1H, broad), 6.82 (2H, broad), 6.99 (1H, broad, Z/E forms), 7.59 (3H, broad), 9.00-9.25 (1H, broad, Z/E forms). LCMS t_{R} (min): 1.42. MS (APCI), m/z 399.03 [M+H]⁺. HPLC t_{R} (min): 9.52. Mₚ 63-65° C.

### 23. 6-Ethoxy-N-(4-methoxy-phenyl)-N′-(1-pyridin-2-yl-ethyl)-[1,3,5]triazine-2,4-diamine

¹H-NMR (400 MHz, DMSO-D₆) δ_{H}: 1.12-1.35 (3H, broad, Z/E forms), 1.48 (3H, t, J=7.5 Hz), 3.72 (3H, s), 4.13-4.35 (2H, broad, Z/E forms), 5.17 (1H, broad, Z/E forms), 6.81 (2H, d, J=8.5 Hz), 7.22 (1H, d/d, J=8.0/5.0 Hz), 7.39 (1H, d, J=8.0 Hz), 7.43 (1H, broad), 7.61 (2H, broad), 7.73 (1H, t, J=8.0 Hz), 8.52 (1H, broad), 9.05-9.15 (1H, broad, Z/E forms) LCMS t_{R} (min): 1.43. MS (ARCI), m/z 367.12 M+H]⁺. HPLC t_{R} (min): 8.84. Mₚ 34-36° C.

### 23. (4-Ethoxy-6-morpholin-4-yl-[1,3,5]triazin-2-yl)-(4-methoxy-phenyl)-amine

¹H-NMR (400 MHz, DMSO-D₆) δ_{H}: 1.28 (3H, t, J=7.5 Hz), 3.64 (4H, m), 3.68 (4H, m), 3.71 (3H, s), 4.30 (2H, q, J=7.5 Hz), 6.87 (2H, d, J=8.5 Hz), 7.75 (2H, d, J=8.5 Hz), 9.22 (1H, broad).

### Generic Synthesis of Intermediates and Final Compounds for Table 20.

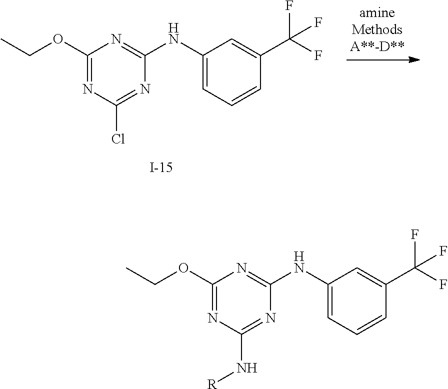

Nucleophilic substitution of the chlorine at I-15 was performed using several general procedures (Methods A**, B**, C**). Basically, the reaction with primary amines was carried out in THF or acetonitrile in the presence of organic bases (DIPEA, TEA, methods A**, B**). The elevated temperatures were required for the reaction with less reactive or sterically hindered aliphatic amines. Interestingly, that in some cases, changing the method B** to the method C** allowed to simplify the work-up procedure and thereby increase the total yield.

| Method | Conditions | Yields |
|---|---|---|
| A** | DIPEA, THF, RT-100^{° C.}, 2-4 h | 7-50% |
| B** | DIPEA or NEt₃, acetonitrile, RT or 50^{° C.} or | 15-85% |
| | refluxing, 3-12 h | |
| C** | K₂CO₃, DMSO, 60-100^{° C.}, 4 h | 36-57% |
| D** | DIPEA or NEt₃, dioxane, 50^{° C.}, 3-16 h | — |

### Generic Procedure for Method A**

A mixture of compound I-15 (500 mg, 1.78 mmol), amine (1.78 mmol), DIPEA (230 mg, 1.78 mmol) and THF (10 mL) was stirred at room temperature or at 50° C. or at 100° C. for 2-4 hours, cooled to room temperature, concentrated and subjected to preparative TLC to give a final product.

Generic Procedure for Method B**

A mixture of compound I-15 (318 mg, 1.0 mmol), corresponding amine (1.1 mmol), DIPEA (0.192 mL, 1.1 mmol) and acetonitrile or NEt₃ (5 mL) was stirred at room temperature or at 50° C. or refluxing for up to 8 hours (TLC control), cooled down to room temperature, diluted with water, filtered or extracted with chloroform. The combined organic phases were concentrated at reduced pressure. Purification by purification by appropriate method furnished a final product.

### Generic Procedure for Method C**

A mixture of compound I-15 478 mg, 1.5 mmol), corresponding amine (1.5 mmol), K₂CO₃ (417 mg, 3.0 mmol) and DMSO (0.2 mL) was stirred at 60-100° C. for 3-16 hours (TLC control), cooled down to room temperature, diluted with water, extracted with dichloromethane. The combined organic phases were concentrated at reduced pressure. Purification by appropriate method furnished a final product.

TABLE 20

| En | Structure | IUPAC name | MW | Formula |
|---|---|---|---|---|
| 1 | 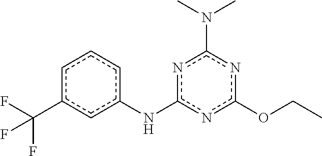 | 6-ethoxy-N,N-dimethyl-N′-[3- (trifluoromethyl)phenyl]-1,3,5- triazine-2,4-diamine | 327.3 | C14H16F3N5O |
| 2 | 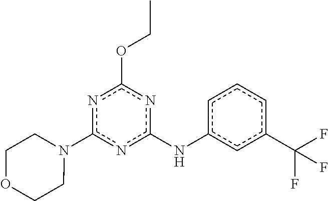 | 4-ethoxy-6-(morpholin-4-yl)-N-[3- (trifluoromethyl)phenyl]-1,3,5- triazin-2-amine | 369.3 | C16H18F3N5O2 |
| 3 | 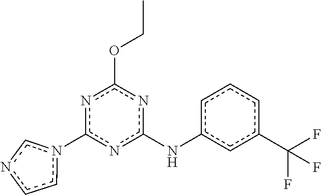 | 4-ethoxy-6-(1H-imidazol-1-yl)-N- [3-(trifluoromethyl)phenyl]-1,3,5- triazin-2-amine | 350.3 | C15H13F3N6O |
| 4 | 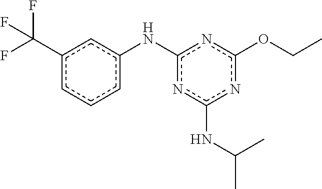 | 6-ethoxy-N-(propan-2-yl)-N′-[3- (trifluoromethyl)phenyl]-1,3,5- triazine-2,4-diamine | 341.3 | C15H18F3N5O |
| 5 | 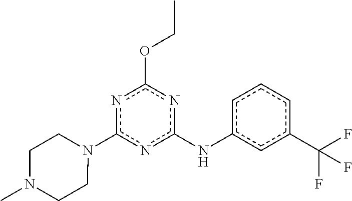 | 4-ethoxy-6-(4-methylpiperazin-1- yl)-N-[3-(trifluoromethyl)phenyl]- 1,3,5-triazin-2-amine | 382.4 | C17H21F3N6O |
| 6 | 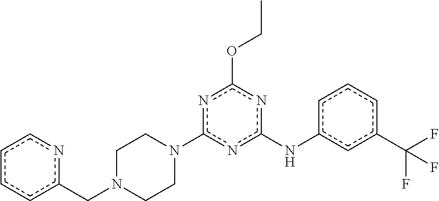 | 4-ethoxy-6-[4-(pyridin-2- ylmethyl)piperazin-1-yl]-N-[3- (trifluoromethyl)phenyl]-1,3,5- triazin-2-amine | 459.5 | C22H24F3N7O |
| 7 | 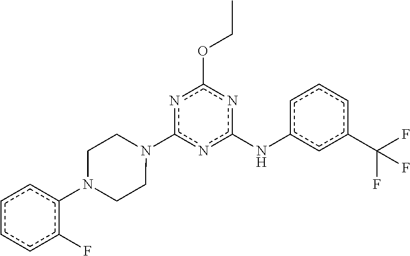 | 4-ethoxy-6-[4-(2- fluorophenyl)piperazin-1-yl]-N-[3- (trifluoromethyl)phenyl]-1,3,5- triazin-2-amine | 462.4 | C22H22F4N6O |
| 8 | 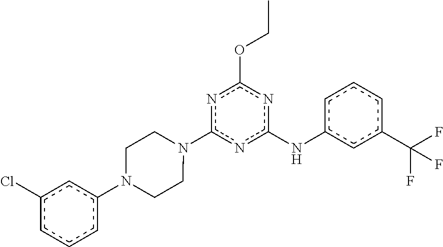 | 4-[4-(3-chlorophenyl)piperazin-1- yl]-6-ethoxy-N-[3- (trifluoromethyl)phenyl]-1,3,5- triazin-2-amine | 478.9 | C22H22ClF3N6O |
| 9 | 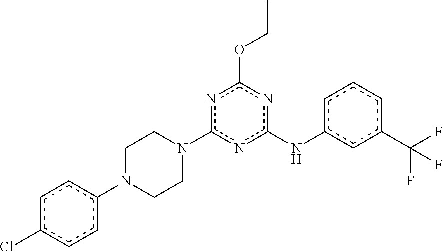 | 4-[4-(4-chlorophenyl)piperazin-1- yl]-6-ethoxy-N-[3- (trifluoromethyl)phenyl]-1,3,5- triazin-2-amine | 478.9 | C22H22ClF3N6O |
| 10 | 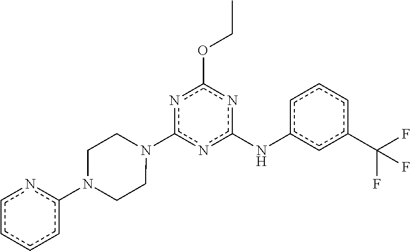 | 4-ethoxy-6-[4-(pyridin-2- yl)piperazin-1-yl]-N-[3- (trifluoromethyl)phenyl]-1,3,5- triazin-2-amine | 445.4 | C21H22F3N7O |
| 11 | 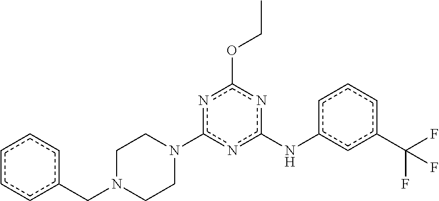 | 4-(4-benzylpiperazin-1-yl)-6- ethoxy-N-[3- (trifluoromethyl)phenyl]-1,3,5- triazin-2-amine | 458.5 | C23H25F3N6O |
| 12 | 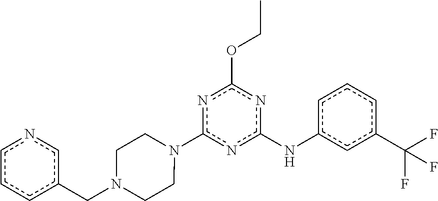 | 4-ethoxy-6-[4-(pyridin-3- ylmethyl)piperazin-1-yl]-N-[3- (trifluoromethyl)phenyl]-1,3,5- triazin-2-amine | 459.5 | C22H24F3N7O |
| 13 | 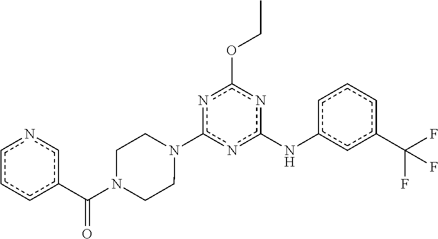 | [4-(4-ethoxy-6-{[3- (trifluoromethyl)phenyl]amino}- 1,3,5-triazin-2-yl)piperazin-1- yl](pyridin-3-yl)methanone | 473.5 | C22H22F3N7O2 |
| 14 | 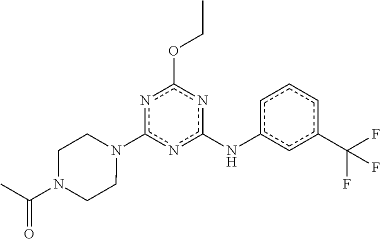 | 1-[4-(4-ethoxy-6-{[3- (trifluoromethyl)phenyl]amino}- 1,3,5-triazin-2-yl)piperazin-1- yl]ethanone | 410.4 | C18H21F3N6O2 |
| 15 | 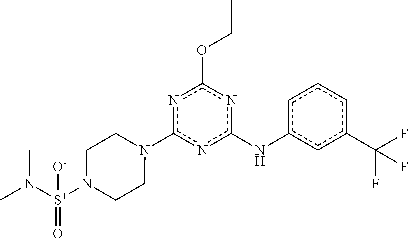 | 4-(4-ethoxy-6-{[3- (trifluoromethyl)phenyl]amino)- 1,3,5-triazin-2-yl)-N,N- dimethylpiperazine-1- sulfonamide | 475.5 | C18H24F3N7O3S |
| 16 | 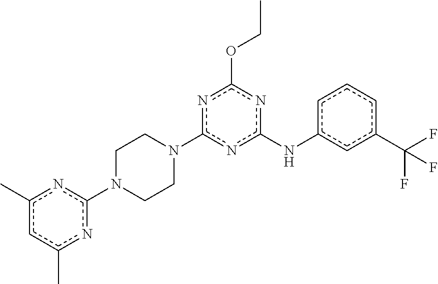 | 4-[4-(4,6-dimethylpyrimidin-2- yl)piperazin-1-yl]-6-ethoxy-N-[3- (trifluoromethyl)phenyl]-1,3,5- triazin-2-amine | 474.5 | C22H25F3N8O |
| 17 | 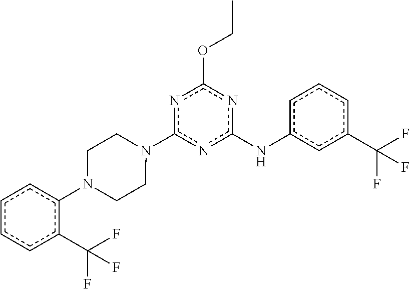 | 4-ethoxy-N-[3- (trifluoromethyl)phenyl]-6-{4-[2- (trifluoromethyl)phenyl]piperazin- 1-yl}-1,3,5-triazin-2-amine | 512.5 | C23H22F6N6O |
| 18 | 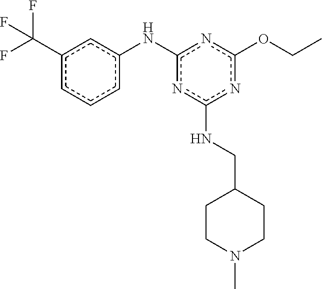 | 6-ethoxy-N-[(1-methylpiperidin-4- yl)methyl]-N′-[3- (trifluoromethyl)phenyl]-1,3,5- triazine-2,4-diamine | 410.4 | C19H25F3N6O |
| 19 | 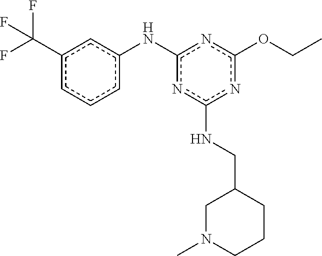 | 6-ethoxy-N-[(1-methylpiperidin-3- yl)methyl]-N′-[3- (trifluoromethyl)phenyl]-1,3,5- triazine-2,4-diamine | 410.4 | C19H25F3N6O |
| 20 | 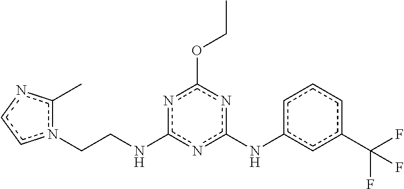 | 6-ethoxy-N-[2-(2-methyl-1H- imidazol-1-yl)ethyl]-N′-[3- (trifluoromethyl)phenyl]-1,3,5- triazine-2,4-diamine | 407.4 | C18H20F3N7O |
| 21 | 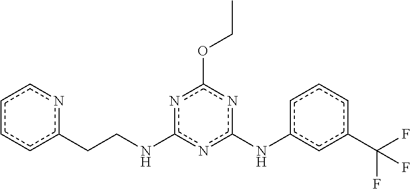 | 6-ethoxy-N-[2-(pyridin-2-yl)ethyl]- N′-[3-(trifluoromethyl)phenyl]- 1,3,5-triazine-2,4-diamine | 404.4 | C19H19F3N6O |
| 22 | 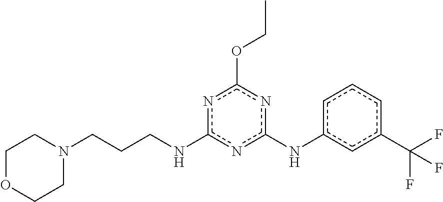 | 6-ethoxy-N-[3-(morpholin-4- yl)propyl]-N′-[3- (trifluoromethyl)phenyl]-1,3,5- triazine-2,4-diamine | 426.4 | C19H25F3N6O2 |
| 23 | 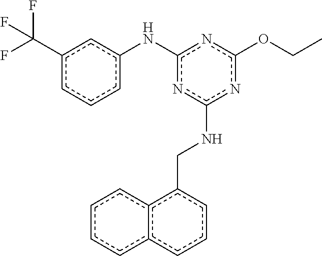 | 6-ethoxy-N-(naphthalen-1- ylmethyl)-N′-[3- (trifluoromethyl)phenyl]-1,3,5- triazine-2,4-diamine | 439.4 | C23H20F3N5O |
| 24 | 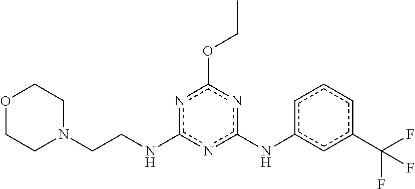 | 6-ethoxy-N-[2-(morpholin-4- yl)ethyl]-N′-[3- (trifluoromethyl)phenyl]-1,3,5- triazine-2,4-diamine | 412.4 | C18H23F3N6O2 |
| 25 | 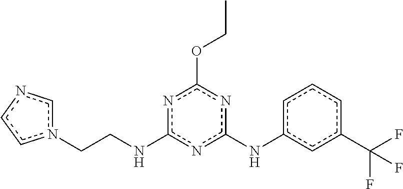 | 6-ethoxy-N-[2-(1H-imidazol-1- yl)ethyl]-N′-[3- (trifluoromethyl)phenyl]-1,3,5- triazine-2,4-diamine | 393.4 | C17H18F3N7O |
| 26 | 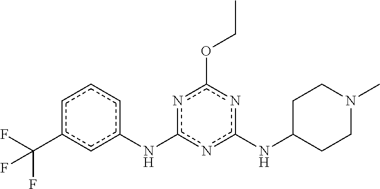 | 6-ethoxy-N-(1-methylpiperidin-4- yl)-N′-[3-(trifluoromethyl)phenyl]- 1,3,5-triazine-2,4-diamine | 396.4 | C18H23F3N6O |
| 27 | 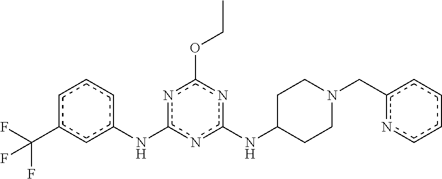 | 6-ethoxy-N-[1-(pyridin-2- ylmethyl)pipendin-4-yl]-N′-[3- (trifluoromethyl)phenyl]-1,3,5- triazine-2,4-diamine | 473.5 | C23H26F3N7O |
| 28 | 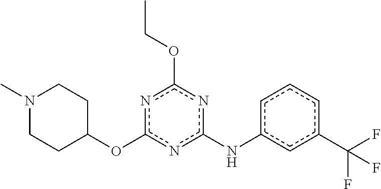 | 4-ethoxy-6-[(1-methylpiperidin-4- yl)oxy]-N-[3- (trifluoromethyl)phenyl]-1,3,5- triazin-2-amine | 397.4 | C18H22F3N5O2 |

### Procedures and Analytical Data for Table 20.

### 1. 6-Ethoxy-N,N-dimethyl-N′-(3-trifluoromethyl-phenyl)-[1,3,5]triazine-2,4-diamine

Method A. Yield 248 mg, 80%. ¹H-NMR (400 MHz, DMSO-D₆) δ_{H}: 1.30 (3H, t, J=7.5 Hz), 3.11 (6H, s), 4.33 (2H, q, J=7.5 Hz), 7.29 (1H, d, J=8.5 Hz), 7.50 (1H, t, J=8.5 Hz), 7.85 (1H, d, J=8.5 Hz), 8.41 (1H, s), 9.71 (1H, s). LCMS t_{R} (min): 2.07. MS (APCI), m/z 328.08 [M+H]⁺. HPLC t_{R} (min): 14.90. Mₚ 84-86° C.

### 2. (4-Ethoxy-6-morpholin-4-yl-[1,3,5]triazin-2-yl)-(3-trifluoromethyl-phenyl)-amine

Method A. Yield 267 mg, 77%. ¹H-NMR (400 MHz, DMSO-D₆) δ_{H}: 1.30 (3H, t, J=7.5 Hz), 3.65 (4H, m), 3.75 (4H, m), 4.35 (2H, q, J=7.5 Hz), 7.30 (1H, d, J=8.5 Hz), 7.50 (1H, t, J=8.5 Hz), 7.86 (1H, d, J=8.5 Hz), 8.30 (1H, s), 9.80 (1H, s). LCMS t_{R} (min): 2.02. MS (APCI), m/z 370.12 [M+H]⁺. HPLC t_{R} (min): 15.02. Mₚ 85-87° C.

### 3. (4-Ethoxy-6-imidazol-1-yl-[1,3,5]triazin-2-yl)-(3-trifluoromethyl-phenyl)-amine

Method B. Yield 235 mg, 67%. ¹H-NMR (400 MHz, DMSO-D₆) δ_{H}: 1.38 (3H, t, J=7.5 Hz), 4.50 (2H, q, J=7.5 Hz), 7.16 (1H, s), 7.43 (1H, d, J=8.5 Hz), 7.61 (1H, t, J=8.5 Hz), 7.83 (1H, s), 7.94 (1H, d, J=8.5 Hz), 8.25 (1H, broad), 8.51 (1H, s), 10.68 (1H, broad). LCMS t_{R} (min): 1.76. MS (APCI), m/z 351.13 [M+H]⁺. HPLC t_{R} (min): 11.53. Mₚ 253-255° C.

### 4. 6-Ethoxy-N-isopropyl-N′-(3-trifluoromethyl-phenyl)-[1,3,5]triazine-2,4-diamine

Method B. Yield 139 mg, 41%. ¹H-NMR (400 MHz, DMSO-D₆) δ_{H}: 1.18 (6H, d, J=7.5 Hz), 1.30 (3H, broad t, J=7.5 Hz), 4.11 (1H, broad m), 4.31 (2H, broad), 7.18-7.40 (1H, broad, Z/E forms), 7.28 (1H, d, J=8.5 Hz), 7.49 (1H, t, J=8.5 Hz), 7.77-8.17 (1H, broad, Z/E forms), 8.05-8.53 (1H, broad, Z/E forms), 9.43-9.73 (1H, broad, Z/E forms). LCMS t_{R} (min): 1.96. MS (APCI), m/z 342.14 [M+H]⁺. HPLC t_{R} (min): 14.30. Mₚ 25-28° C.

### 5. [4-Ethoxy-6-(4-methyl-piperazin-1-yl)-[1,3,5]triazin-2-yl]-(3-trifluoromethyl-phenyl)-amine

Method B. Yield 196 mg, 54%. ¹H-NMR (400 MHz, DMSO-D₆) δ_{H}: 1.30 (3H, t, J=7.5 Hz), 2.21 (3H, s), 2.38 (4H, broad multiplet), 3.75 (4H, broad multiplet), 4.32 (2H, q, J=7.5 Hz), 7.30 (1H, d, J=8.5 Hz), 7.50 (1H, t, J=8.5 Hz), 7.82 (1H, d, J=8.5 Hz), 8.32 (1H, broad), 9.78 (1H, broad). LCMS t_{R} (min): 1.51. MS (APCI), m/z 383.14 [M+H]⁺. HPLC t_{R} (min): 10.84. Mₚ 49-51° C.

### 6. [4-Ethoxy-6-(4-pyridin-2-ylmethyl-piperazin-1-yl)-[1,3,5]triazin-2-yl]-(3-trifluoromethyl-phenyl)-amine

Method A. Yield 385 mg, 52%. ¹H-NMR (400 MHz, DMSO-D₆) δ_{H}: 1.29 (3H, t, J=7.5 Hz), 3.25 (4H, m), 3.66 (2H, s), 3.78 (4H, broad), 4.32 (2H, q, J=7.5 Hz), 7.27 (2H, m), 7.49 (2H, m), 7.78 (1H, t, J=8.0 Hz), 7.82 (1H, d, J=8.0 Hz), 8.30 (1H, broad), 8.50 (1H, broad), 9.77 (1H, broad). LCMS t_{R} (min): 1.61. MS (APCI), m/z 460.17 [M+H]⁺. HPLC t_{R} (min): 11.46. Mₚ 72-75° C.

### 7. {4-Ethoxy-6-[4-(2-fluoro-phenyl)-piperazin-1-yl]-[1,3,5]triazin-2-yl}-(3-trifluoromethyl-phenyl)-amine

Method A. Yield 255 mg, 37%. ¹H-NMR (400 MHz, DMSO-D₆) δ_{H}: 1.32 (3H, t, J=7.5 Hz), 3.08 (4H, m), 3.91 (4H, m), 4.35 (2H, q, J=7.5 Hz), 7.00 (1H, broad m), 7.11 (3H, m), 7.30 (1H, d, J=8.5 Hz), 7.52 (1H, t, J=8.5 Hz), 7.87 (1H, d, J=8.5 Hz), 8.30 (1H, s), 9.81 (1H, broad). LCMS t_{R} (min): 2.32. MS (APCI), m/z 463.14 [M+H]⁺. HPLC t_{R} (min): 17.90. Mₚ 118-120° C.

### 8. {4-[4-(3-Chloro-phenyl)-piperazin-1-yl]-6-ethoxy-[1,3,5]triazin-2-yl}-(3-trifluoromethyl-phenyl)-amine

Method A. Yield 424 mg, 89%. ¹H-NMR (400 MHz, DMSO-D₆) δ_{H}: 1.31 (3H, t, J=7.5 Hz), 3.28 (4H, m), 3.89 (4H, broad), 4.36 (2H, q, J=7.5 Hz), 6.80 (1H, d, J=8.5 Hz), 6.93 (1H, d, J=8.5 Hz), 7.00 (1H, s), 7.22 (1H, t, J=8.5 Hz), 7.31 (1H, d, J=8.5 Hz), 7.52 (1H, t, J=8.5 Hz), 7.88 (1H, d, J=8.5 Hz), 8.30 (1H, s), 9.82 (1H, broad). LCMS t_{R} (min): 2.39. MS (APCI), m/z 479.14, 481.13 [M+H]⁺. HPLC t_{R} (min): 18.42. Mₚ 170-172° C.

### 9. {4-[4-(4-Chloro-phenyl)-piperazin-1-yl]-6-ethoxy-[1,3,5]triazin-2-yl}-(3-trifluoromethyl-phenyl)-amine

Method B. Yield 350 mg, 73%. ¹H-NMR (400 MHz, DMSO-D₆) δ_{H}: 1.32 (3H, t, J=7.5 Hz), 3.25 (4H, m), 3.90 (4H, m), 4.34 (2H, q, J=7.5 Hz), 7.00 (2H, d, J=8.5 Hz), 7.24 (2H, d, J=8.5 Hz), 7.31 (1H, d, J=8.5 Hz), 7.56 (1H, t, J=8.5 Hz), 7.73 (1H, d, J=8.5 Hz), 8.30 (1H, s), 9.80 (1H, broad). LCMS t_{R} (min): 2.38. MS (APCI), m/z 479.12, 481.13 [M+H]⁺. HPLC t_{R} (min): 18.32. Mₚ 198-200° C.

### 10. [4-Ethoxy-6-(4-pyridin-2-yl-piperazin-1-yl)-[1,3,5]triazin-2-yl]-(3-trifluoromethyl-phenyl)-amine

Method B. Yield 384 mg, 86%. ¹H-NMR (400 MHz, DMSO-D₆) δ_{H}: 1.32 (3H, t, J=7.5 Hz), 3.60 (4H, m), 3.88 (4H, m), 4.36 (2H, q, J=7.5 Hz), 6.67 (1H, t, J=8.5 Hz), 6.88 (1H, d, J=8.5 Hz), 7.31 (1H, d, J=8.5 Hz), 7.52 (2H, m), 7.86 (1H, d, J=8.5 Hz), 8.23 (1H, d, J=5.0 Hz), 8.32 (1H, broad), 9.81 (1H, broad). LCMS t_{R} (min): 1.70. MS (APCI), m/z 446.15 [M+H]⁺. HPLC t_{R} (min): 11.62. Mₚ 179-181° C.

### 11. [4-(4-Benzyl-piperazin-1-yl)-6-ethoxy-[1,3,5]triazin-2-yl]-(3-trifluoromethyl-phenyl)-amine

Method B. Yield 364 mg, 79%. ¹H-NMR (400 MHz, DMSO-D₆) δ_{H}: 1.30 (3H, t, J=7.5 Hz), 2.42 (4H, m), 3.52 (2H, s), 3.74 (4H, m), 4.32 (2H, q, J=7.5 Hz), 7.28 (1H, d, J=8.5 Hz), 7.32 (5H, m), 7.50 (1H, t, J=8.5 Hz), 7.83 (1H, d, J=8.5 Hz), 8.30 (1H, broad), 9.77 (1H, broad). LCMS t_{R} (min): 1.97. MS (APCI), m/z 459.13 [M+H]⁺. HPLC t_{R} (min): 12.39. Mₚ 158-160° C.

### 12. [4-Ethoxy-6-(4-pyridin-3-ylmethyl-piperazin-1-yl)-[1,3,5]triazin-2-yl]-(3-trifluoromethyl-phenyl)-amine

Yield 70 mg, 15%. ¹H-NMR (400 MHz, DMSO-D₆) δ_{H}: 1.30 (3H, t, J=7.5 Hz), 2.43 (4H, m), 3.57 (2H, s), 3.76 (4H, m), 4.31 (2H, q, J=7.5 Hz), 7.28 (1H, d, J=8.5 Hz), 7.37 (1H, dd, J=8.0, 5.0 Hz), 7.50 (1H, t, J=8.5 Hz), 7.73 (1H, d, J=8.0 Hz), 7.82 (1H, d, J=8.5 Hz), 8.29 (1H, s), 8.48 (1H, d, J=5.0 Hz), 8.52 (1H, s), 8.78 (1H, broad). LCMS t_{R} (min): 1.61. MS (APCI), m/z 460.16 [M+H]⁺. HPLC t_{R} (min): 12.13. Mₚ 158-160° C.

### 13. {4-[4-Ethoxy-6-(3-trifluoromethyl-phenylamino)-[1,3,5]-triazin-2-yl]-piperazin-1-yl}-pyridin-3-yl-methanone

Yield 135 mg, 29%. ¹H-NMR (400 MHz, DMSO-D₆) δ_{H}: 1.30 (3H, t, J=7.5 Hz), 3.60 (4H, broad), 3.83 (4H, m), 4.34 (2H, q, J=7.5 Hz), 7.31 (1H, dd, J=8.0, 5.0 Hz), 7.50 (2H, broad), 7.89 (2H, superposition of two signals), 8.28 (1H, broad), 8.69 (2H, superposition of two signals), 9.82 (1H, s). LCMS t_{R} (min): 1.84. MS (APCI), m/z 474.16 [M+H]⁺. HPLC t_{R} (min): 12.13. M_{P} 128-129° C.

### 14. 1-{-4-[4-Ethoxy-6-(3-trifluoromethyl-phenylamino)-[1,3,5]triazin-2-yl]-piperazin-1-yl}-ethanone

Method B. Yield 300 mg, 73%. ¹H-NMR (400 MHz, DMSO-D₆) δ_{H}: 1.30 (3H, t, J=7.5 Hz), 2.05 (3H, s), 3.52 (4H, m), 3.73 (2H, broad), 3.79 (2H, broad), 4.34 (2H, q, J=7.5 Hz), 7.31 (1H, d, J=8.5 Hz), 7.51 (1H, t, J=8.5 Hz), 7.86 (1H, d, J=8.5 Hz), 8.29 (1H, broad), 9.82 (1H, broad). LCMS t_{R} (min): 1.83. MS (APCI), m/z 411.13 [M+H]⁺. HPLC t_{R} (min): 11.45. Mₚ 172-174° C.

### 15. 4-[4-Ethoxy-6-(3-trifluoromethyl-phenylamino)-[1,3,5]triazin-2-yl]-piperazine-1-sulfonic acid dimethylamide

Method B. Yield 444 mg, 93%. ¹H-NMR (400 MHz, DMSO-D₆) δ_{H}: 1.31 (3H, t, J=7.5 Hz), 2.80 (6H, s), 3.25 (4H, m), 3.82 (4H, broad), 4.35 (2H, q, J=7.5 Hz), 7.32 (1H, d, J=8.5 Hz), 7.52 (1H, t, J=8.5 Hz), 7.88 (1H, d, J=8.5 Hz), 8.28 (1H, s), 9.85 (1H, broad). LCMS t_{R} (min): 2.04. MS (APCI), m/z 476.14 [M+H]⁺. HPLC t_{R} (min): 15.52. Mₚ 129-131° C.

### 16. {4-[4-(4,6-Dimethyl-pyrimidin-2-yl)-piperazin-1-yl]-6-ethoxy-[1,3,5]triazin-2-yl}-(3-trifluoromethyl-phenyl)-amine

Method C. Yield 218 mg, 45%. ¹H-NMR (400 MHz, DMSO-D₆) δ_{H}: 1.32 (3H, t, J=7.5 Hz), 2.50 (6H, s), 3.82 (8H, m), 4.36 (2H, q, J=7.5 Hz), 6.42 (1H, s), 7.30 (1H, d, J=8.5 Hz), 7.52 (1H, t, J=8.5 Hz), 7.88 (1H, d, J=8.5 Hz), 8.33 (1H, s), 9.81 (1H, s). LCMS t_{R} (min): 2.28. MS (APCI), m/z 475.16 [M+H]⁺. HPLC t_{R} (min): 14.10. Mₚ 233-235° C.

### 17. {4-Ethoxy-6-[4-(2-trifluoromethyl-phenyl)-piperazin-1-yl]-[1,3,5]triazin-2-yl}-(3-trifluoromethyl-phenyl)-amine

Method A. Yield 256 mg, 50%. ¹H-NMR (400 MHz, DMSO-D₆) δ_{H}: 1.31 (3H, t, J=7.5 Hz), 2.92 (4H, m), 3.90 (4H, m), 4.36 (2H, q, J=7.5 Hz), 7.29 (1H, d, J=8.5 Hz), 7.36 (1H, t, J=8.5 Hz), 7.52 (1H, t, J=8.5 Hz), 7.59 (1H, d, J=8.5 Hz), 7.66 (1H, t, J=8.5 Hz), 7.70 (1H, d, J=8.5 Hz), 7.87 (1H, d, J=8.5 Hz), 8.31 (1H, s), 9.82 (1H, broad). LCMS t_{R} (min): 2.46. MS (APCI), m/z 513.16 [M+H]⁺. HPLC t_{R} (min): 18.99. Mₚ 167-169° C.

### 18. 6-Ethoxy-N-(1-methyl-piperidin-4-ylmethyl)-N′-(3-trifluoromethyl-phenyl)-[1,3,5]triazine-2,4-diamine

Yield 40 mg, 7%. ¹H-NMR (400 MHz, DMSO-D₆) δ_{H}: 1.18 (2H, m), 1.28 (3H, broad triplet, J=7.5 Hz), 1.53 (1H, broad), 1.63 (2H, m), 1.90 (2H, broad), 2.19 (3H, broad), 2.80 (2H, broad), 3.40 (2H, broad), 4.31 (2H, broad q, J=7.5 Hz), 7.27 (1H, broad), 7.40-7.58 (1H, broad, Z/E forms), 7.47 (1H, t, J=8.5 Hz), 7.80-8.08 (1H, two broad doublets, J=8.5 Hz, Z/E forms), 8.10-8.45 (1H, broad, Z/E forms), 9.50-9.75 (1H, broad, Z/E forms). LCMS t_{R} (min): 1.56. MS (APCI), m/z 411.18 [M+H]⁺. HPLC t_{R} (min): 10.31. Mₚ 104-106° C.

### 19. 6-Ethoxy-N-(1-methyl-piperidin-3-ylmethyl)-N′-(3-trifluoromethyl-phenyl)-[1,3,5]triazine-2,4-diamine

Yield 272 mg, 71%. ¹H-NMR (400 MHz, DMSO-D₆) δ_{H}: 0.90 (1H, m), 1.30 (3H, broad), 1.42 (1H, m), 1.62 (3H, broad), 1.85 (2H, broad), 2.12 (3H, s), 2.52-2.74 (2H, superposition of two doublets, J=7.5 Hz, Z/E forms), 3.25 (2H, m), 4.30 (2H, broad), 7.28 (1H, broad), 7.42-7.57 (1H, broad, Z/E forms), 7.48 (1H, t, J=8.5 Hz), 7.80-8.10 (1H, broad doublets, J=8.5 Hz, Z/E forms), 8.12-8.45 (1H, broad, Z/E forms), 9.51-9.72 (1H, broad, Z/E forms). LCMS t_{R} (min): 1.56. MS (APCI), m/z 411.24 [M+H]⁺. HPLC t_{R} (min): 10.41. Mₚ 157-159° C.

### 20. 6-Ethoxy-N-[2-(2-methyl-imidazol-1-yl)-ethyl]-N′-(3-trifluoromethyl-phenyl)-[1,3,5]triazine-2,4-diamine

Yield 164 mg, 43%. ¹H-NMR (400 MHz, DMSO-D₆) δ_{H}: 1.30 (3H, broad), 2.15-2.30 (3H, two singlets, Z/E forms), 3.58 (2H, broad), 4.07 (2H, broad triplet, J=7.5 Hz), 4.32 (2H, broad), 6.70 (1H, broad), 6.98 (1H, broad), 7.30 (1H, broad), 7.50 (1H, broad doublet, J=8.5 Hz), 7.50-7.60 (1H, broad, Z/E forms), 7.82-8.05 (1H, broad doublets, J=8.5 Hz, Z/E forms), 8.13-8.33 (1H, broad, Z/E forms), 9.60-9.78 (1H, broad, Z/E forms). LCMS t_{R} (min): 1.56. MS (APCI), m/z 408.05 [M+H]⁺. HPLC t_{R} (min): 10.47. M_{P} 181-183° C.

### 21. 6-Ethoxy-N-(2-pyridin-2-yl-ethyl)-N′-(3-trifluoromethyl-phenyl)-[1,3,5]-triazine-2,4-diamine

Yield 380 mg, 77%. ¹H-NMR (400 MHz, DMSO-D₆) δ_{H}: 1.30 (3H, broad), 3.02 (2H, broad triplet, J=7.5 Hz), 3.68 (2H, broad), 4.32 (2H, broad triplet, J=7.5 Hz), 7.22 (1H, m), 7.29 (2H, m), 7.48 (1H, t, J=8.5 Hz), 7.50-7.58 (1H, broad, Z/E forms), 7.70 (1H, broad), 7.90-8.05 (1H, superposition of two doublets, J=8.5 Hz, Z/E forms), 8.13-8.37 (1H, broad, Z/E forms), 8.50 (1H, broad), 9.52-9.78 (1H, broad, Z/E forms). LCMS t_{R} (min): 1.60. MS (APCI), m/z 405.10 [M+H]⁺. HPLC t_{R} (min): 10.51. M_{P} 98-100° C.

### 22. 6-Ethoxy-N-(3-morpholin-4-yl-propyl)-N′-(3-trifluoromethyl-phenyl)-[1,3,5]triazine-2,4-diamine

Yield 362 mg, 85%. ¹H-NMR (400 MHz, DMSO-D₆) δ_{H}: 1.30 (3H, broad), 1.70 (2H, broad), 2.33 (6H, m), 3.33 (2H, broad), 3.58 (4H, broad), 4.32 (2H, broad), 7.29 (1H, broad), 7.38-7.50 (1H, broad, Z/E forms), 7.51 (1H, broad), 7.82-8.09 (1H, broad, Z/E forms), 8.12-8.48 (1H, broad, Z/E forms), 9.50-9.74 (1H, broad, Z/E forms). LCMS t_{R} (min): 1.57. MS (APCI), m/z 427.13 [M+H]⁺. HPLC t_{R} (min): 10.34. M_{P} 177-179° C.

### 23. 6-Ethoxy-N-naphthalen-1-ylmethyl-N′-(3-trifluoromethyl-phenyl)-[1,3,5]triazine-2,4-diamine

Yield 247 mg, 56%. ¹H-NMR (400 MHz, DMSO-D₆) δ_{H}: 1.18-1.33 (3H, superposition of two broad triplets, J=7.5 Hz, Z/E forms), 4.30 (2H, broad quartet, J=7.5 Hz), 4.91-5.08 (2H, broad, Z/E forms), 7.19 (1H, broad), 7.26 (1H, broad), 7.45 (2H, broad), 7.53 (2H, broad), 7.80 (2H, broad), 7.92 (1H, d, J=8.5 Hz), 8.05 (1H, broad), 8.13 (1H, broad), 8.18 (1H, broad), 9.60-9.74 (1H, broad, Z/E forms). LCMS t_{R} (min): 2.19. MS (APCI), m/z 440.11 [M+H]⁺. HPLC t_{R} (min): 16.51. M_{P} 77-79° C.

### 24. 6-Ethoxy-N-(2-morpholin-4-yl-ethyl)-N′-(3-trifluoromethyl-phenyl)-[1,3,5]triazine-2,4-diamine

Yield 87 mg, 21%. ¹H-NMR (400 MHz, DMSO-D₆) δ_{H}: 1.30 (3H, broad), 2.40 (4H, m), 2.48 (2H, m), 3.41 (2H, broad), 3.58 (4H, m), 4.32 (2H, broad), 7.30 (2H, broad), 7.50 (1H, t, J=8.5 Hz), 7.84-8.08 (1H, broad doublets, J=8.5 Hz, Z/E forms), 8.12-8.39 (1H, broad, Z/E forms), 9.53-9.74 (1H, broad, Z/E forms). LCMS t_{R} (min): 1.54. MS (APCI), m/z 413.12 [M+H]⁺. HPLC t_{R} (min): 10.69. M_{P} 138-139° C.

### 25. 6-Ethoxy-N-(2-imidazol-1-yl-ethyl)-N′-(3-trifluoromethyl-phenyl)-[1,3,5]triazine-2,4-diamine

A mixture of compound 15 (343 mg, 1.09 mmol), 2-imidazol-1-yl-ethylamine (200 mg, 1.09 mmol), triethylamine (0.31 mL, 2.20 mmol) and acetonitrile (6 mL) was stirred at room temperature for 24 hours, diluted with water. The formed solid was collected by filtration, washed with water and purified by column chromatography (silica gel, ethanol/ethyl acetate) giving the compound. Yield 124 mg, 29%. ¹H-NMR (400 MHz, DMSO-D₆) δ_{H}: 1.27 (3H, broad), 3.60 (2H, broad), 4.14 (2H, broad), 4.30 (2H, broad), 6.84 (1H, s), 7.10 (1H, broad), 7.27 (1H, broad), 7.46 (1H, broad), 7.47-7.55 (1H, broad, Z/E forms), 7.55 (1H, s), 7.82-8.02 (1H, broad, Z/E forms), 8.10-8.30 (1H, broad, Z/E forms), 9.55-9.78 (1H, broad, Z/E forms). LCMS t_{R} (min): 1.51. MS (APCI), m/z 394.00 [M+H]⁺. HPLC t_{R} (min): 10.29. M_{P} ° C.

### 26. 6-Ethoxy-N-pyridin-2-ylmethyl-N′-(3-trifluoromethyl-phenyl)-[1,3,5]triazine-2,4-diamine

Yield 285 mg, 57%. ¹H-NMR (400 MHz, DMSO-D₆) δ_{H}: 1.18-1.37 (3H, broad, Z/E forms), 4.20-4.40 (2H, broad, Z/E forms), 4.62 (2H, broad), 7.22 (2H, broad), 7.30 (1H, broad), 7.39-7.52 (1H, broad Z/E forms), 7.73 (1H, broad), 7.79-8.00 (1H, broad Z/E forms), 7.90-8.11 (1H, broad Z/E forms), 8.20 (1H, s), 8.50 (1H, broad), 9.62-9.77 (1H, broad, Z/E forms). LCMS t_{R} (min): 1.64. MS (APCI), m/z 391.11 [M+H]⁺. HPLC t_{R} (min): 10.73. M_{P} 150-152° C.

### 27. 6-Ethoxy-N-(1-methyl-piperidin-4-yl)-N′-(3-trifluoromethyl-phenyl)-[1,3,5]triazine-2,4-diamine hydrochloride*HCl

¹H-NMR (400 MHz, DMSO-D₆) δ_{H}: 1.30 (3H, broad triplet, J=7.5 Hz), 1.95 (2H, m), 2.00-2.15 (2H, broad, Z/E forms), 2.71 (3H, broad), 2.93-3.12 (2H, broad, Z/E forms), 3.20-3.50 (2H, broad, Z/E forms), 3.95 (1H, broad), 4.38 (2H, broad q, J=7.5 Hz), 7.32 (1H, broad), 7.52 (1H, broad triplet, J=7.5 Hz), 7.69-7.90 (1H, broad, Z/E forms), 7.90-8.12 (1H, broad, Z/E forms), 8.12-8.38 (1H, broad, Z/E forms), 9.68-10.00 (1H, broad, Z/E forms), 10.70-10.90 (1H, broad, Z/E forms). LCMS t_{R} (min): 1.57. MS (APCI), m/z 397.14 [M+H]⁺. HPLC t_{R} (min): 10.35. M_{P} 95-97° C.

### 28. 6-Ethoxy-N-(1-pyridin-2-ylmethyl-piperidin-4-yl)-N′-(3-trifluoromethyl-phenyl)-[1,3,5]triazine-2,4-diamine hydrochloride *HCl

Yield 200 mg, 41%. ¹H-NMR (400 MHz, DMSO-D₆) δ_{H}: 1.30 (3H, broad triplet, J=7.5 Hz), 1.95 (2H, broad multiplet), 2.11 (2H, broad), 3.15 (2H, broad), 3.45 (2H, broad), 4.01 (1H, broad), 4.31 (2H, broad quartet, J=7.5 Hz), 4.45 (2H, broad), 7.30 (1H, d, J=8.5 Hz), 7.50 (2H, broad), 7.55-7.78 (1H, broad, Z/E forms), 7.70 (1H, broad), 7.85-8.10 (1H, broad, Z/E forms), 7.94 (1H, t, J=8.5 Hz), 8.10-8.32 (1H, broad, Z/E forms), 8.69 (1H, broad), 9.53-9.81 (1H, broad, Z/E forms), 10.66 (1H, broad). LCMS t_{R} (min): 1.61. MS (APCI), m/z 474.20 [M+H]⁺. HPLC t_{R} (min): 11.01. M_{P} 21

### Generic Synthesis of Intermediates and Final Compounds for Table 21 (see Table 19).

TABLE 21

| En | Structure | IUPAC name | MW | Formula |
|---|---|---|---|---|
| 1 | 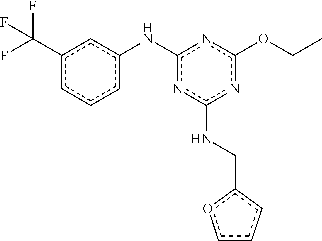 | 6-ethoxy-N-(furan-2-ylmethyl)- N′-[3-(trifluoromethyl)phenyl]- 1,3,5-triazine-2,4-diamine | 379.3 | C17H16F3N5O2 |
| 2 | 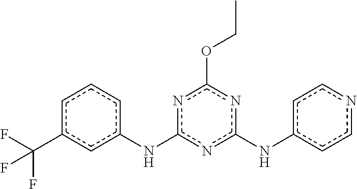 | 6-ethoxy-N-(pyridin-4-yl)-N′-[3- (trifluoromethyl)phenyl]-1,3,5- triazine-2,4-diamine | 376.3 | C17H15F3N6O |
| 3 | 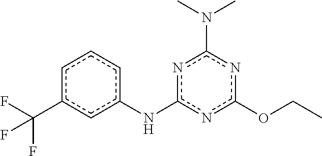 | 6-ethoxy-N,N-dimethyl-N′-[3- (trifluoromethyl)phenyl]-1,3,5- triazine-2,4-diamine | 327.3 | C14H16F3N5O |
| 4 | 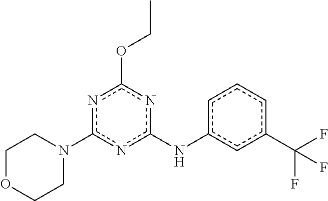 | 4-ethoxy-6-(morpholin-4-yl)-N- [3-(trifluoromethyl)phenyl]- 1,3,5-triazin-2-amine | 369.3 | C16H18F3N5O2 |
| 5 | 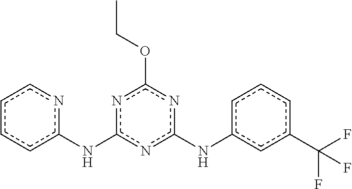 | 6-ethoxy-N-(pyridin-2-yl)-N′-[3- (trifluoromethyl)phenyl]-1,3,5- triazine-2,4-diamine | 376.3 | C17H15F3N6O |
| 6 | 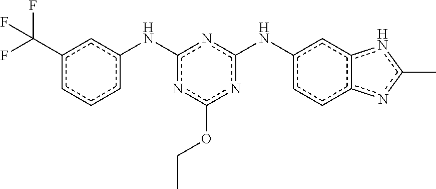 | 6-ethoxy-N-(2-methyl-1H- benzimidazol-6-yl)-N′-[3- (trifluoromethyl)phenyl]-1,3,5- triazine-2,4-diamine | 429.4 | C20H18F3N7O |
| 7 | 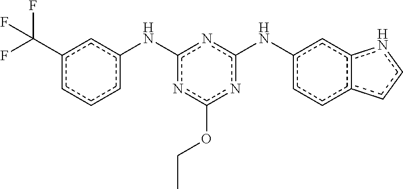 | 6-ethoxy-N-(1H-indol-6-yl)-N′- [3-(trifluoromethyl)phenyl]- 1,3,5-triazine-2,4-diamine | 414.4 | C20H17F3N6O |
| 8 | 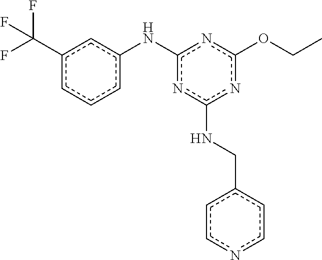 | 6-ethoxy-N-(pyridin-4-ylmethyl)- N′-[3-(trifluoromethyl)phenyl]- 1,3,5-triazine-2,4-diamine | 390.4 | C18H17F3N6O |
| 9 | 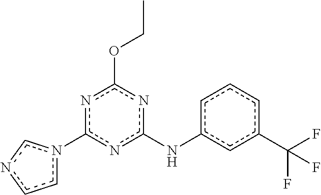 | 4-ethoxy-6-(1H-imidazol-1-yl)- N-[3-(trifluoromethyl)phenyl]- 1,3,5-triazin-2-amine | 350.3 | C15H13F3N6O |
| 10 | 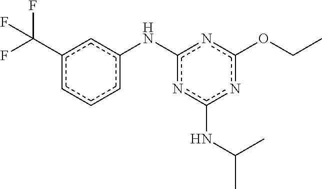 | 6-ethoxy-N-(propan-2-yl)-N′-[3- (trifluoromethyl)phenyl]-1,3,5- triazine-2,4-diamine | 341.3 | C15H18F3N5O |
| 11 | 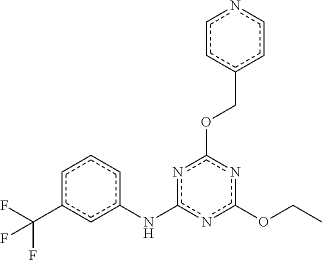 | 4-ethoxy-6-(pyridin-4- ylmethoxy)-N-[3- (trifluoromethyl)phenyl]-1,3,5- triazin-2-amine | 391.3 | C18H16F3N5O2 |
| 12 | 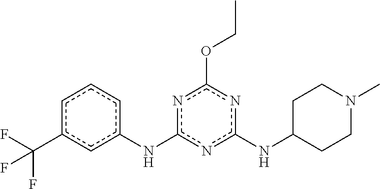 | 6-ethoxy-N-(1-methylpiperidin- 4-yl)-N′-[3- (trifluoromethyl)phenyl]-1,3,5- triazine-2,4-diamine | 396.4 | C18H23F3N6O |
| 13 | 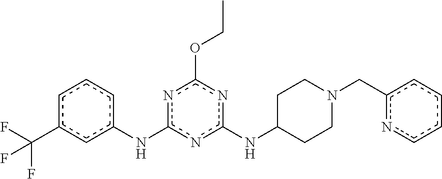 | 6-ethoxy-N-[1-(pyridin-2- ylmethyl)piperidin-4-yl]-N′-[3- (trifluoromethyl)phenyl]-1,3,5- triazine-2,4-diamine | 473.5 | C23H26F3N7O |
| 14 | 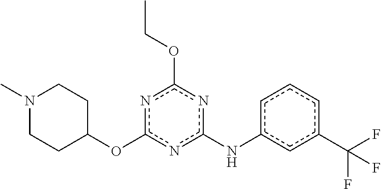 | 4-ethoxy-6-[(1-methylpiperidin- 4-yl)oxy]-N-[3- (trifluoromethyl)phenyl]-1,3,5- triazin-2-amine | 397.4 | C18H22F3N5O2 |
| 15 | 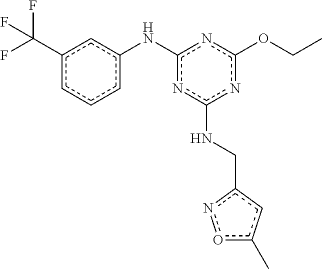 | 6-ethoxy-N-[(5-methylisoxazol- 3-y)methyl]-N′-[3- (trifluoromethyl)phenyl]-1,3,5- triazine-2,4-diamine | 394.4 | C17H17F3N6O2 |
| 16 | 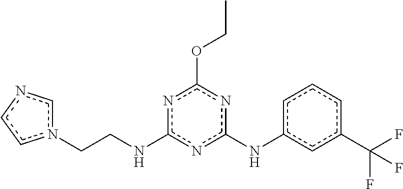 | 6-ethoxy-N-[2-(1H-imidazol-1- yl)ethyl]-N′-[3- (trifluoromethyl)phenyl]-1,3,5- triazine-2,4-diamine | 393.4 | C17H18F3N7O |
| 17 | 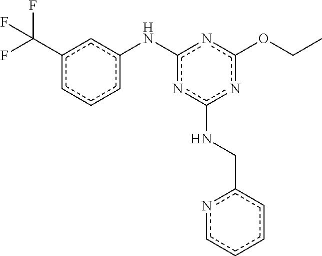 | 6-ethoxy-N-(pyridin-2-ylmethyl)- N′-[3-(trifluoromethyl)phenyl]- 1,3,5-triazine-2,4-diamine | 390.4 | C18H17F3N6O |
| 18 | 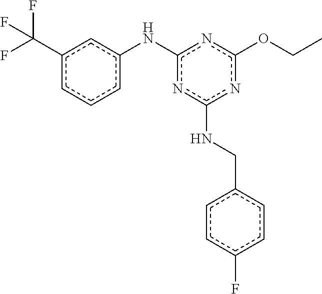 | 6-ethoxy-N-(4-fluorobenzyl)-N′- [3-(trifluoromethyl)phenyl]- 1,3,5-triazine-2,4-diamine | 407.4 | C19H17F4N5O |
| 19 | 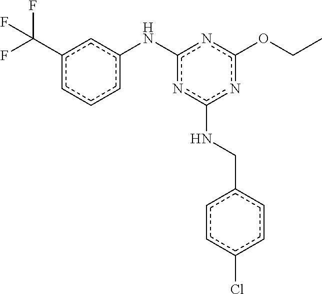 | N-(4-chlorobenzyl)-6-ethoxy-N′- [3-(trifluoromethyl)phenyl]- 1,3,5-triazine-2,4-diamine | 423.8 | C19H17C1F3N5O |
| 20 | 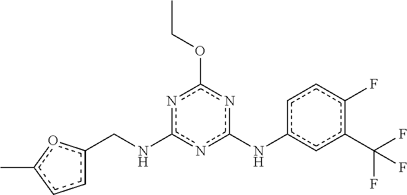 | 6-ethoxy-N-[4-fluoro-3- (trifluoromethyl)phenyl]-N′-[(5- methylfuran-2-yl)methyl]-1,3,5- triazine-2,4-diamine | 411.4 | C18H17F4N5O2 |
| 21 | 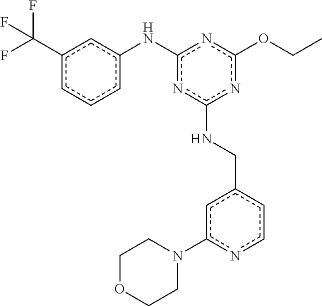 | 6-ethoxy-N-([2-(morpholin-4- yl)pyridin-4-yl]methyl}-N′-[3- (trifluoromethyl)phenyl]-1,3,5- triazine-2,4-diamine | 475.5 | C22H24F3N7O2 |

### Procedures and Analytical Data for Table 21.

### 1. 6-Ethoxy-N-furan-2-ylmethyl-N′-(3-trifluoromethyl-phenyl)-[1,3,5]triazine-2,4-diamine

¹H-NMR (400 MHz, DMSO-D₆) δ_{H}: 1.30 (3H, t, J=7.5 Hz), 4.30 (2H, broad), 4.51 (2H, broad), 6.22 (1H, broad), 6.37 (1H, broad), 7.28 (1H, d, J=8.5 Hz), 7.46 (1H, t, J=8.5 Hz), 7.50 (1H, s), 7.80-8.10 (2H, broad, Z/E forms), 8.10-8.40 (1H, broad, Z/E forms), 9.55-9.80 (1H, broad, Z/E forms). LCMS t_{R} 2.00 (min). MS (APCI), m/z 379.95 [M+H]⁺. Mₚ 147-149° C.

### 2. 6-Ethoxy-N-pyridin-4-yl-N′-(3-trifluoromethyl-phenyl)-[1,3,5]triazine-2,4-diamine

¹H-NMR (400 MHz, DMSO-D₆) δ_{H}: 1.36 (3H, t, J=7.5 Hz), 4.42 (2H, q, J=7.5 Hz), 7.38 (1H, d, J=7.5 Hz), 7.58 (1H, t, J=7.5 Hz), 7.78 (2H, broad), 7.96-8.03 (1H, broad), 8.00 (1H, d, J=8.5 Hz), 7.98-8.19 (1H, broad, Z/E forms), 8.38 (2H, d, J=5.0 Hz), 10.01 (1H, broad). LCMS t_{R} (min): 1.56. MS (APCI), m/z 376.98 [M+H]⁺. HPLC t_{R} (min): 11.15. Mₚ 245-247° C.

### 3. 6-Ethoxy-N,N-dimethyl-N′-(3-trifluoromethyl-phenyl)-[1,3,5]triazine-2,4-diamine

¹H-NMR (400 MHz, DMSO-D₆) δ_{H}: 1.30 (3H, t, J=7.5 Hz), 3.11 (6H, s), 4.33 (2H, q, J=7.5 Hz), 7.29 (1H, d, J=8.5 Hz), 7.50 (1H, t, J=8.5 Hz), 7.85 (1H, d, J=8.5 Hz), 8.41 (1H, s), 9.71 (1H, s). LCMS t_{R} (min): 2.07. MS (APCI), m/z 328.08 [M+H]⁺. HPLC t_{R} (min): 14.90. Mₚ 84-86° C.

### 4. (4-Ethoxy-6-morpholin-4-yl-[1,3,5]triazin-2-yl)-(3-trifluoromethyl-phenyl)-amine

¹H-NMR (400 MHz, DMSO-D₆) δ_{H}: 1.30 (3H, t, J=7.5 Hz), 3.65 (4H, m), 3.75 (4H, m), 4.35 (2H, q, J=7.5 Hz), 7.30 (1H, d, J=8.5 Hz), 7.50 (1H, t, J=8.5 Hz), 7.86 (1H, d, J=8.5 Hz), 8.30 (1H, s), 9.80 (1H, s). LCMS t_{R} (min): 2.02. MS (APCI), m/z 370.12 [M+H]⁺. HPLC t_{R} (min): 15.02. Mₚ 85-87° C.

### 5. 6-Ethoxy-N-pyridin-2-yl-N′-(3-trifluoromethyl-phenyl)-[1,3,5]triazine-2,4-diamine

¹H-NMR (400 MHz, DMSO-D₆) δ_{H}: 1.35 (3H, t, J=7.5 Hz), 4.41 (2H, q, J=7.5 Hz), 7.08 (1H, d/d, J=8.0/5.0 Hz), 7.35 (1H, d, J=8.5 Hz), 7.54 (1H, t, J=8.5 Hz), 7.77 (1H, t, J=8.5 Hz), 8.08 (1H, broad), 8.20 (2H, broad), 8.32 (1H, broad), 9.78 (1H, broad), 9.98 (1H, broad). LCMS t_{R} (min): 1.65. MS (APCI), m/z 376.98 [M+H]⁺. HPLC t_{R} (min): 11.42. Mₚ 72-74° C.

### 6. 6-Ethoxy-N-(2-methyl-3H-benzoimidazol-5-yl)-N′-(3-trifluoromethyl-phenyl)-[1,3,5]triazine-2,4-diamine

Yield 29 mg, 7%. ¹H-NMR (400 MHz, DMSO-D₆) δ_{H}: 1.32 (3H, t, J=7.5 Hz), 4.40 (2H, q, J=7.5 Hz), 7.30 (1H, d, J=8.5 Hz), 7.37 (3H, broad), 7.50 (2H, broad), 7.78 (1H, broad), 7.92-8.15 (1H, broad, Z/E forms), 8.12 (2H, broad), 9.48 (1H, broad), 9.68 (1H, broad), 11.96 (1H, broad). LCMS t_{R} (min): 1.60. MS (APCI), m/z 430.04 [M+H]⁺. HPLC t_{R} (min): 11.23. Mₚ 116-118° C.

### 7. 6-Ethoxy-N-(1H-indol-6-yl)₄β-(3-trifluoromethyl-phenyl)-[1,3,5]triazine-2,4-diamine

¹H-NMR (400 MHz, DMSO-D₆) δ_{H}: 1.33 (3H, t, J=7.5 Hz), 4.40 (2H, q, J=7.5 Hz), 6.39 (1H, broad), 7.28 (1H, s), 7.45 (2H, m), 7.48 (2H, m), 7.65 (1H, broad), 8.12 (2H, broad), 9.47 (1H, broad), 9.73 (1H, broad), 10.90 (1H, broad). LCMS t_{R} (min): 2.00. MS (APCI), m/z 415.01 [M+H]⁺. HPLC t_{R} (min): 15.34. Mₚ 91-93° C.

### 8. 6-Ethoxy-N-(2-morpholin-4-yl-ethyl)-N′-(3-trifluoromethyl-phenyl)-[1,3,5]triazine-2,4-diamine (34k)

¹H-NMR (400 MHz, DMSO-D₆) δ_{H}: 1.30 (3H, broad), 2.40 (4H, m), 2.48 (2H, m), 3.41 (2H, broad), 3.58 (4H, m), 4.32 (2H, broad), 7.30 (2H, broad), 7.50 (1H, t, J=8.5 Hz), 7.84-8.08 (1H, broad doublets, J=8.5 Hz, Z/E forms), 8.12-8.39 (1H, broad, Z/E forms), 9.53-9.74 (1H, broad, Z/E forms). LCMS t_{R} (min): 1.54. MS (APCI), m/z 413.12 [M+H]⁺. HPLC t_{R} (min): 10.69. M_{P} 138-139° C.

### 9. (4-Ethoxy-6-imidazol-1-yl-[1,3,5]triazin-2-yl)-(3-trifluoromethyl-phenyl)-amine

Yield 235 mg, 67%. ¹H-NMR (400 MHz, DMSO-D₆) δ_{H}: 1.38 (3H, t, J=7.5 Hz), 4.50 (2H, q, J=7.5 Hz), 7.16 (1H, s), 7.43 (1H, d, J=8.5 Hz), 7.61 (1H, t, J=8.5 Hz), 7.83 (1H, s), 7.94 (1H, d, J=8.5 Hz), 8.25 (1H, broad), 8.51 (1H, s), 10.68 (1H, broad). LCMS t_{R} (min): 1.76. MS (APCI), m/z 351.13 [M+H]⁺. HPLC t_{R} (min): 11.53. Mₚ 253-255° C.

### 10. 6-Ethoxy-N-isopropyl-N′-(3-trifluoromethyl-phenyl)-[1,3,5]triazine-2,4-diamine

Yield 139 mg, 41%. ¹H-NMR (400 MHz, DMSO-D₆) δ_{H}: 1.18 (6H, d, J=7.5 Hz), 1.30 (3H, broad t, J=7.5 Hz), 4.11 (1H, broad m), 4.31 (2H, broad), 7.18-7.40 (1H, broad, Z/E forms), 7.28 (1H, d, J=8.5 Hz), 7.49 (1H, t, J=8.5 Hz), 7.77-8.17 (1H, broad, Z/E forms), 8.05-8.53 (1H, broad, Z/E forms), 9.43-9.73 (1H, broad, Z/E forms). LCMS t_{R} (min): 1.96. MS (APCI), m/z 342.14 [M+H]⁺. HPLC t_{R} (min): 14.30. Mₚ 25-28° C.

### 11. [4-Ethoxy-6-(pyridin-4-ylmethoxy)-[1,3,5]triazin-2-yl]-(3-trifluoromethyl-phenyl)-amine

Yield 160 mg, 27%. ¹H-NMR (400 MHz, DMSO-D₆) δ_{H}: 1.31 (3H, t, J=7.5 Hz), 4.40 (2H, q, J=7.5 Hz), 5.49 (2H, s), 7.40 (3H, m), 7.56 (1H, t, J=8.5 Hz), 7.90 (1H, d, J=8.5 Hz), 8.20 (1H, broad), 8.60 (2H, d, J=5.0 Hz), 10.34 (1H, broad). LCMS t_{R} (min): 1.58. MS (APCI), m/z 391.92 [M+H]⁺. HPLC t_{R} (min): 10.94. M_{P} 94-96° C.

### 12. 6-Ethoxy-N-(1-methyl-piperidin-4-yl)-N′-(3-trifluoromethyl-phenyl)-[1,3,5]triazine-2,4-diamine hydrochloride *HCl

¹H-NMR (400 MHz, DMSO-D₆) δ_{H}: 1.30 (3H, broad triplet, J=7.5 Hz), 1.95 (2H, m), 2.00-2.15 (2H, broad, Z/E forms), 2.71 (3H, broad), 2.93-3.12 (2H, broad, Z/E forms), 3.20-3.50 (2H, broad, Z/E forms), 3.95 (1H, broad), 4.38 (2H, broad q, J=7.5 Hz), 7.32 (1H, broad), 7.52 (1H, broad triplet, J=7.5 Hz), 7.69-7.90 (1H, broad, Z/E forms), 7.90-8.12 (1H, broad, Z/E forms), 8.12-8.38 (1H, broad, Z/E forms), 9.68-10.00 (1H, broad, Z/E forms), 10.70-10.90 (1H, broad, Z/E forms). LCMS t_{R} (min): 1.57. MS (APCI), m/z 397.14 [M+H]⁺. HPLC t_{R} (min): 10.35. M_{P} 95-97° C.

### 13. 6-Ethoxy-N-(1-pyridin-2-ylmethyl-piperidin-4-yl)-N′-(3-trifluoromethyl-phenyl)-[1,3,5]triazine-2,4-diamine hydrochloride *HCl

¹H-NMR (400 MHz, DMSO-D₆) δ_{H}: 1.30 (3H, broad triplet, J=7.5 Hz), 1.95 (2H, broad multiplet), 2.11 (2H, broad), 3.15 (2H, broad), 3.45 (2H, broad), 4.01 (1H, broad), 4.31 (2H, broad quartet, J=7.5 Hz), 4.45 (2H, broad), 7.30 (1H, d, J=8.5 Hz), 7.50 (2H, broad), 7.55-7.78 (1H, broad, Z/E forms), 7.70 (1H, broad), 7.85-8.10 (1H, broad, Z/E forms), 7.94 (1H, t, J=8.5 Hz), 8.10-8.32 (1H, broad, Z/E forms), 8.69 (1H, broad), 9.53-9.81 (1H, broad, Z/E forms), 10.66 (1H, broad). LCMS t_{R} (min): 1.61. MS (APCI), m/z 474.20 [M+H]⁺. HPLC t_{R} (min): 11.01. M_{P} 21 14. [4-Ethoxy-6-(1-methyl-piperidin-4-yloxy)-[1,3,5]triazin-2-yl]-(3-trifluoromethyl-phenyl)-amine hydrochloride *HCl

¹H-NMR (400 MHz, DMSO-D₆) δ_{H}: 1.32 (3H, t, J=7.5 Hz), 2.03 (1H, m), 2.20 (2H, m), 2.30 (1H, m), 2.69-2.80 (3H, broad, Z/E forms), 3.10 (2H, m), 3.32 (1H, m), 3.48 (1H, m), 4.40 (2H, q, J=8.5 Hz), 5.06-5.33 (1H, broad, Z/E forms), 7.40 (1H, d, J=8.5 Hz), 7.59 (1H, broad triplet, J=8.5 Hz), 7.88-8.00 (1H, superposition of two doublets, J=8.5 Hz, Z/E forms), 8.10-8.25 (1H, broad, Z/E forms), 10.30 (1H, broad), 10.91 (1H, broad). LCMS t_{R} (min): 1.58. MS (APCI), m/z 397.94 [M+H]⁺. HPLC t_{R} (min): 10.79. M_{P} 88-90° C.

### 15. 6-Ethoxy-N-(5-methyl-isoxazol-3-ylmethyl)-N′-(3-trifluoromethyl-phenyl)-[1,3,5]triazine-2,4-diamine

¹H-NMR (400 MHz, DMSO-D₆) δ_{H}: 1.30 (3H, broad), 2.34 (3H, s), 4.32 (2H, q, J=7.5 Hz), 4.52 (2H, broad), 6.15 (1H, s), 7.30 (1H, d, J=8.5 Hz), 7.50 (1H, broad triplet, J=8.5 Hz), 7.90 (1H, broad), 8.00 (1H, broad), 8.16-8.30 (1H, broad Z/E forms), 9.65-9.81 (1H, broad Z/E forms). LCMS t_{R} (min): 1.97. MS (APCI), m/z 395.08 [M+H]⁺. HPLC t_{R} (min): 14.21. M_{P} 153-155° C.

### 16. 6-Ethoxy-N-(2-imidazol-1-yl-ethyl)-N′-(3-trifluoromethyl-phenyl)-[1,3,5]triazine-2,4-diamine

A mixture of compound 15 (343 mg, 1.09 mmol), 2-imidazol-1-yl-ethylamine (200 mg, 1.09 mmol), triethylamine (0.31 mL, 2.20 mmol) and acetonitrile (6 mL) was stirred at room temperature for 24 hours, diluted with water. The formed solid was collected by filtration, washed with water and purified by column chromatography (silica gel, ethanol/ethyl acetate) giving the compound. Yield 124 mg, 29%. ¹H-NMR (400 MHz, DMSO-D₆) δ_{H}: 1.27 (3H, broad), 3.60 (2H, broad), 4.14 (2H, broad), 4.30 (2H, broad), 6.84 (1H, s), 7.10 (1H, broad), 7.27 (1H, broad), 7.46 (1H, broad), 7.47-7.55 (1H, broad, Z/E forms), 7.55 (1H, s), 7.82-8.02 (1H, broad, Z/E forms), 8.10-8.30 (1H, broad, Z/E forms), 9.55-9.78 (1H, broad, Z/E forms). LCMS t_{R} (min): 1.51. MS (APCI), m/z 394.00 [M+H]⁺. HPLC t_{R} (min): 10.29. M_{P} ° C.

### 17. 6-Ethoxy-N-pyridin-2-ylmethyl-N′-(3-trifluoromethyl-phenyl)-[1,3,5]triazine-2,4-diamine

¹H-NMR (400 MHz, DMSO-D₆) δ_{H}: 1.18-1.37 (3H, broad, Z/E forms), 4.20-4.40 (2H, broad, Z/E forms), 4.62 (2H, broad), 7.22 (2H, broad), 7.30 (1H, broad), 7.39-7.52 (1H, broad Z/E forms), 7.73 (1H, broad), 7.79-8.00 (1H, broad Z/E forms), 7.90-8.11 (1H, broad Z/E forms), 8.20 (1H, s), 8.50 (1H, broad), 9.62-9.77 (1H, broad, Z/E forms). LCMS t_{R} (min): 1.64. MS (APCI), m/z 391.11 [M+H]⁺. HPLC t_{R} (min): 10.73. M_{P} 150-152° C.

### 18. 6-Ethoxy-N-(4-fluoro-benzyl)-N′-(3-trifluoromethyl-phenyl)-[1,3,5]triazine-2,4-diamine

¹H-NMR (400 MHz, DMSO-D₆) δ_{H}: 1.25 (3H, broad t, J=7.5 Hz, Z/E forms), 4.30 (2H, broad), 4.48-4.52 (2H, two broad signals, Z/E forms), 7.10 (2H, broad m), 7.25 (1H, d, J=8.5 Hz), 7.33 (2H, broad), 7.45 (1H, broad d, J=8.5 Hz), 7.81-7.95 (1H, two broad d, J=8.5 Hz, Z/E forms), 8.03 (1H, broad), 8.25-8.30 (1H, two broad signals, Z/E forms), 9.63-9.75 (1H, two broad signals, Z/E forms). MW 407.37. LCMS t_{R} (min): 2.05. MS (APCI) m/z 408.07 [M+H]⁺. HPLC t_{R} (min): 15.84. M_{P} 85-87° C.

### 19. N-(4-Chloro-benzyl)-6-ethoxy-N′-(3-trifluoromethyl-phenyl)-[1,3,5]triazine-2,4-diamine

¹H-NMR (400 MHz, DMSO-D₆) δ_{H}: 1.28 (3H, superposition of two t, J=7.5 Hz, Z/E forms), 4.30 (2H, broad, Z/E forms), 4.45-4.55 (2H, two broad signals, Z/E forms), 7.26 (1H, broad, Z/E forms), 7.35 (4H, broad), 7.45 (1H, broad, Z/E forms), 7.81-7.97 (1H, two broad signals, Z/E forms), 8.05 (1H, broad, Z/E forms), 8.17-8.24 (1H, two broad signals, Z/E forms), 9.65-9.73 (1H, two broad signals, Z/E forms). MW 423.83. LCMS t_{R} (min): 2.13. MS (APCI), m/z 424.02 [M+H]⁺. HPLC t_{R} (min): 16.64. M_{P} 146-148° C.

### 20. 6-Ethoxy-N-(2-pyridin-2-yl-ethyl)-N′-(3-trifluoromethyl-phenyl)-[1,3,5]triazine-2,4-diamine

¹H-NMR (400 MHz, DMSO-D₆) δ_{H}: 1.30 (3H, broad), 3.02 (2H, broad triplet, J=7.5 Hz), 3.68 (2H, broad), 4.32 (2H, broad triplet, J=7.5 Hz), 7.22 (1H, m), 7.29 (2H, m), 7.48 (1H, t, J=8.5 Hz), 7.50-7.58 (1H, broad, Z/E forms), 7.70 (1H, broad), 7.90-8.05 (1H, superposition of two doublets, J=8.5 Hz, Z/E forms), 8.13-8.37 (1H, broad, Z/E forms), 8.50 (1H, broad), 9.52-9.78 (1H, broad, Z/E forms). LCMS t_{R} (min): 1.60. MS (APCI), m/z 405.10 [M+H]⁺. HPLC t_{R} (min): 10.51. M_{P} 98-100° C.

### 21. 2. 6-Ethoxy-N-(3-morpholin-4-yl-propyl)-N′-(3-trifluoromethyl-phenyl)-[1,3,5]triazine-2,4-diamine

¹H-NMR (400 MHz, DMSO-D₆) δ_{H}: 1.30 (3H, broad), 1.70 (2H, broad), 2.33 (6H, m), 3.33 (2H, broad), 3.58 (4H, broad), 4.32 (2H, broad), 7.29 (1H, broad), 7.38-7.50 (1H, broad, Z/E forms), 7.51 (1H, broad), 7.82-8.09 (1H, broad, Z/E forms), 8.12-8.48 (1H, broad, Z/E forms), 9.50-9.74 (1H, broad, Z/E forms). LCMS t_{R} (min): 1.57. MS (APCI), m/z 427.13 [M+H]⁺. HPLC t_{R} (min): 10.34. M_{P} 177-179° C.

### Generic Synthesis of Intermediates and Final Compounds for Table 22

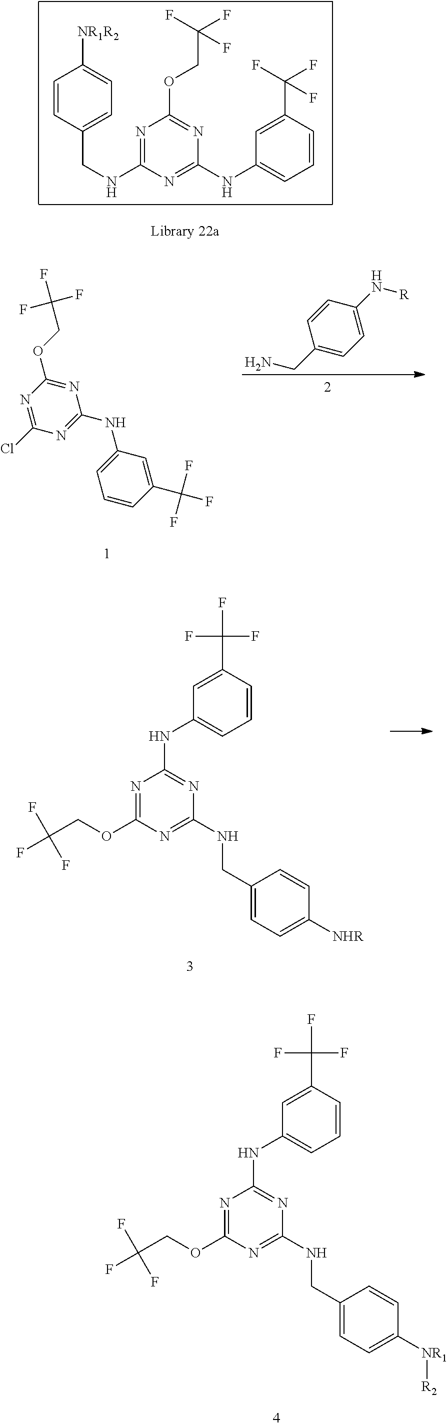

Intermediate 3 was obtained according to the following procedure: A mixture of 2 (0.1 mol) and triazine 1 (0.1 mol) in dioxane was treated with triethylamine (0.18 mol) at 50° C. for 5 hours. The solvent was removed under reduced pressure and the residue was washed with water, filtered, dried, and purified by using column chromatography with CH₂Cl₂ as eluent to give intermediate 3 as white solid.

Intermediate 4 was obtained according to the following procedure: A solution of 10 mmol of 3 in 10 ml of CH₂Cl₂ was treated with acyl chloride (10 mmol) and triethylamine (22 mmol). This mixture was stirred at r.t. for 3 hours, then cooled and evaporated. The residue was treated with water. The solvent was removed and the product purified by column chromatography.

A solution of 10 mmol of 3 in 10 ml of dioxane was treated with sulphochloride (10 mmol) and triethylamine (22 mmol). This mixture was stirred at reflux for 3 hours, then cooled and evaporated. The residue was treated with water. The resulting precipitate was filtered, washed with water, dried, and purified by column chromatography.

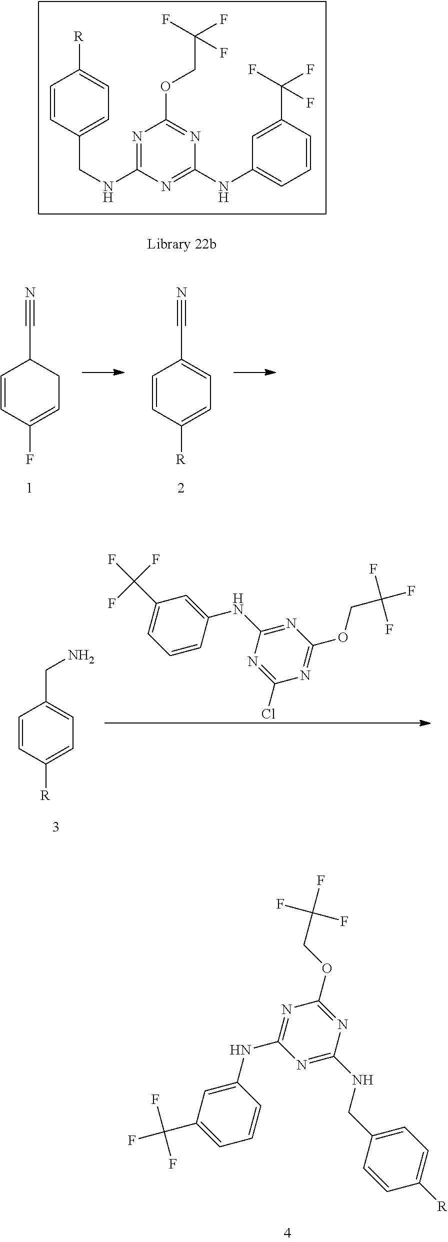

Intermediate 2 was obtained according to the following procedure: A mixture of intermediates 1 (10 g) and amine (15.38 g) in the presence of potassium carbonate (11.41 g) was stirred in DMSO (100 ml) at 100° C. for 5 hours, after which it was cooled, and treated with 500 ml of water, filtered, and washed with IPA and hexane providing intermediate 2 as yellow solid.

Intermediate 3 was obtained according to the following procedure: Intermediate 2 (2 g.) was dissolved in THF (132 ml) and hydrogenated in the presence of Raney nickel (2 g) and 25% aqueous ammonia (9 ml) under 2 atm at r.t. After completion of the reaction, the catalyst was removed by filtration, and the solvent was removed under reduced pressure and pure intermediate 3 was obtained as white solid. Yield 1.53 g (75%).

Intermediate 4 was obtained according to the following procedure: A mixture of intermediate 3 (1.53 g) and monochlorotriazine (2 g) in dioxane was treated with triethylamine (0.8 g) at 50° C. for 5 hours. The solvent was removed under reduced pressure and the residue was washed with water, filtered, dried, and purified by using column chromatography with CH₂Cl₂ as eluent to give intermediate 4 as white solid.

TABLE 22

| En | Structure | IUPAC Name | MW | Formula |
|---|---|---|---|---|
| 1 | 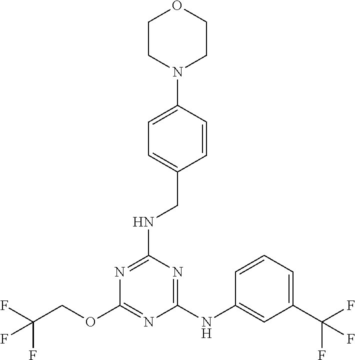 | N-[4-(morpholin-4-yl)benzyl]-6- (2,2,2-trifluoroethoxy)-N′-[3- (trifluoromethyl)phenyl]-1,3,5- triazine-2,4-diamine | 528.5 | C23H22F6N6O2 |
| 2 | 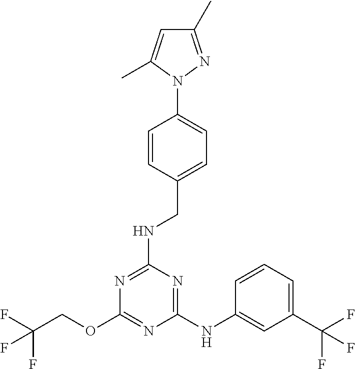 | N-[4-(3,5-dimethyl-1H-pyrazol-1- yl)benzyl]-6-(2,2,2-trifluoroethoxy)- N′-[3-(trifluoromethyl)-phenyl]- 1,3,5-triazine-2,4-diamine | 537.5 | C24H21F6N7O |
| 3 | 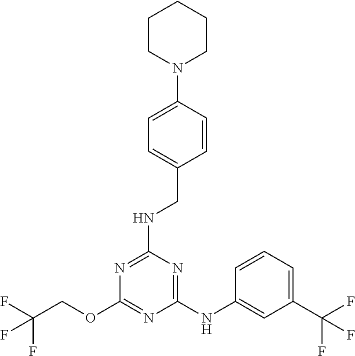 | N-[4-(piperidin-1-yl)benzyl]-6- (2,2,2-trifluoroethoxy)-N′-[3- (trifluoromethyl)phenyl]-1,3,5- triazine-2,4-diamine | 526.5 | C24H24F6N6O |
| 4 | 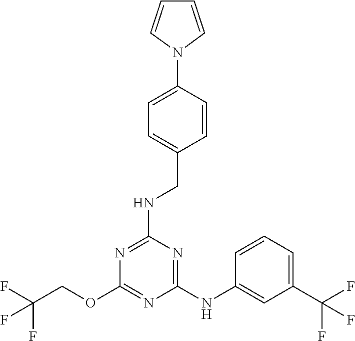 | N-[4-(1H-pyrrol-1-yl)benzyl]-6- (2,2,2-trifluoroethoxy)-N′-[3- (trifluoromethyl)phenyl]-1,3,5- triazine-2,4-diamine | 508.4 | C23H18F6N6O |
| 5 | 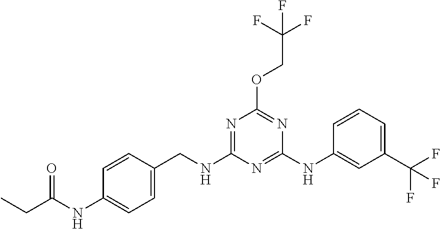 | N-[4-({[4-(2,2,2-trifluoroethoxy)-6- {[3-(trifluoromethyl)phenyl]amino}- 1,3,5-triazin-2- yl]amino}methyl)phenyl]furan-2- carboxamide | 514.4 | C22H20F6N6O2 |
| 6 | 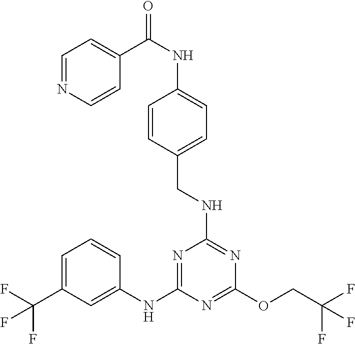 | N-(4-{[(4-(2,2,2-trifluoroethoxy)-6- {[3-(trifluoromethyl)phenyl]amino]- 1,3,5-triazin-2- yl)amino]methyl}phenyl)isonicotinamide | 563.5 | C25H19F6N7O2 |
| 7 | 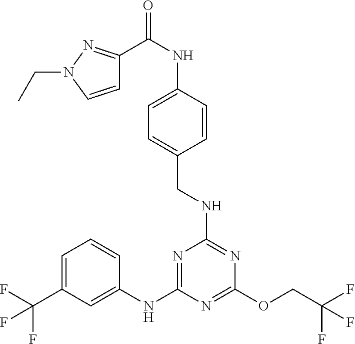 | 1-ethyl-N-(4-{[(4-(2,2,2- trifluoroethoxy)-6-{[3- (trifluoromethyl)phenyl]amino}- 1,3,5-triazin-2-yl)amino]methyl}phenyl)- 1H-pyrazole-3-carboxamide | 580.5 | C25H22F6N8O2 |
| 8 | 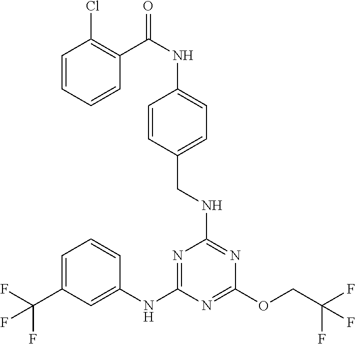 | 2-chloro-N-(4-{[(4-(2,2,2- trifluoroethoxy)-6-{[3- (trifluoromethyl)phenyl]amino)- 1,3,5-triazin-2- yl)amino]methyl}phenyl)benzamide | 596.9 | C26H19ClF6N6O |
| 9 | 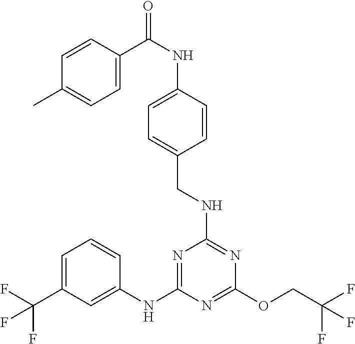 | 4-methyl-N-(4-{[(4-(2,2,2- trifluoroethoxy)-6-{[3- (trifluoromethyl)phenyl]amino}- 1,3,5-triazin-2- yl)amino]methyl}phenyl)benzamide | 576.5 | C27H22F6N6O2 |
| 10 | 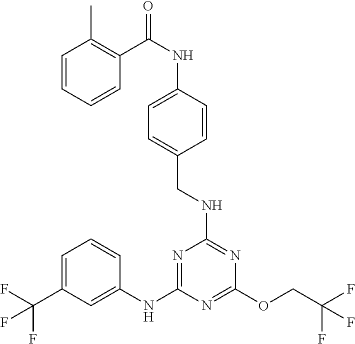 | 2-methyl-N-(4-{[(4-(2,2,2- trifluoroethoxy)-6-{[3- (trifluoromethyl)phenyl]amino]- 1,3,5-triazin-2- yl)amino]methyl}phenyl)benzamide | 576.5 | C27H22F6N6O2 |
| 11 | 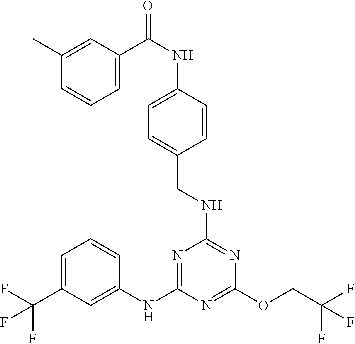 | 3-methyl-N-(4-{[(4-(2,2,2- trifluoroethoxy)-6-{[3- (trifluoromethyl)phenyl]amino}- 1,3,5-triazin-2- yl)amino]methyl}phenyl)benzamide | 576.5 | C27H22F6N6O2 |
| 12 | 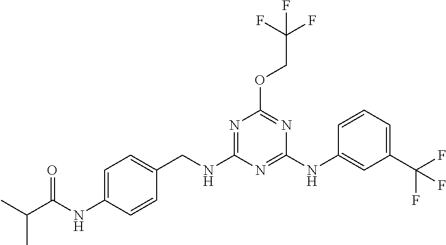 | 2-methyl-N-(4-{[(4-(2,2,2- trifluoroethoxy)-6-{[3- (trifluoromethyl)phenyl]amino)-1,3,5- triazin-2- yl)amino]methyl}phenyl)propanamide | 528.5 | C23H22F6N6O2 |
| 13 | 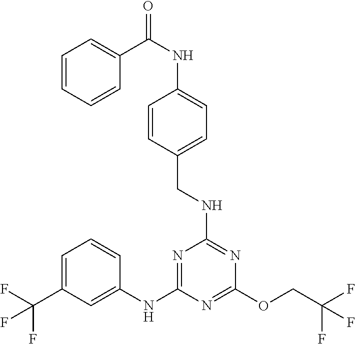 | N-(4-{[(4-(2,2,2-trifluoroethoxy)-6- {[3-(trifluoromethyl)phenyl]amino)- 1,3,5-triazin-2- yl)amino]methyl}phenyl)benzamide | 562.5 | C26H20F6N6O2 |
| 14 | 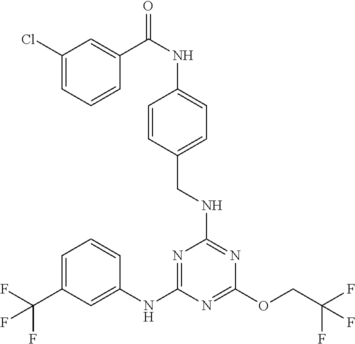 | 3-chloro-N-(4-{[(4-(2,2,2- trifluoroethoxy)-6-{[3- (trifluoromethyl)phenyl]amino}- 1,3,5-triazin-2- yl)amino]methyl}phenyl)benzamide | 596.9 | C26H19ClF6N6O |
| 15 | 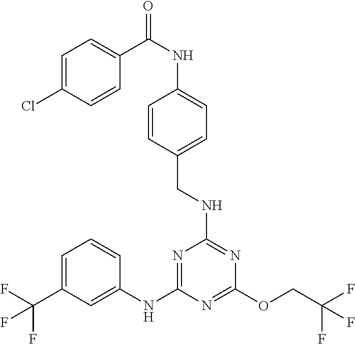 | 4-chloro-N-(4-{[(4-(2,2,2- trifluoroethoxy)-6-{[3- (trifluoromethyl)phenyl]amino}- 1,3,5-triazin-2- yl)amino]methyl}phenyl)benzamide | 596.9 | C26H19ClF6N6O |
| 16 | 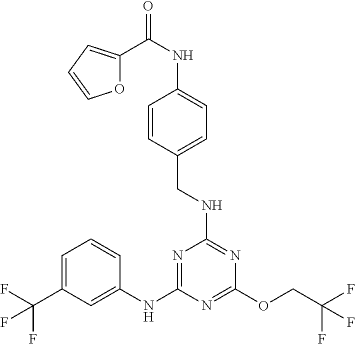 | N-(4-{[(4-(2,2,2-trifluoroethoxy)-6- {[3-(trifluoromethyl)phenyl]amino}- yl)amino]methyl}phenyl)-2- furamide | 552.4 | C24H18F6N6O3 |
| 17 | 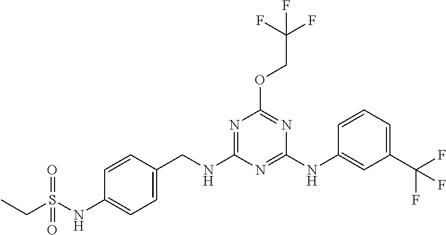 | N-(4-{[(4-(2,2,2-trifluoroethoxy)-6- {[3-(trifluoromethyl)phenyl]amino}- 1,3,5-triazin-2- yl)amino]methyl}phenyl)ethanesulf onamide | 551.5 | C21H20F6N6O3 |
| 18 | 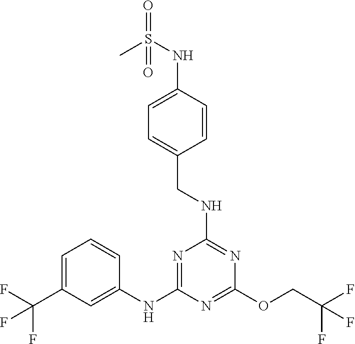 | N-(4-{[(4-(2,2,2-trifluoroethoxy)-6- {[3-(trifluoromethyl)phenyl]amino}- 1,3,5-triazin-2- yl)amino]methyl}phenyl)methanesu lfonamide | 537.5 | C20H18F6N6O3 |
| 19 | 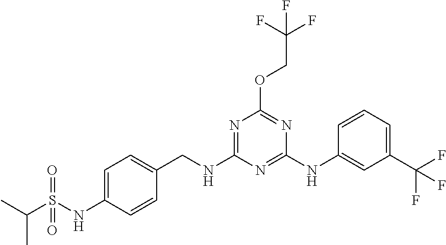 | N-(4-{[(4-(2,2,2-trifluoroethoxy)-6-{[3- (trifluoromethyl)phenyl]amino}-1,3,5- triazin-2- yl)amino]methyl}phenyl)propane-2- sulfonamide | 565.5 | C22H22F6N6O3 |
| 20 | 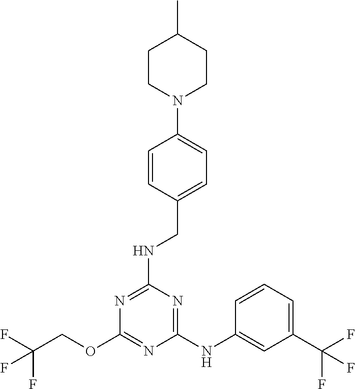 | N-[4-(4-methylpiperidin-1- yl)benzyl]-6-(2,2,2-trifluoroethoxy)- N′-[3-(trifluoromethyl)phenyl]-1,3,5- triazine-2,4-diamine | 540.5 | C25H26F6N6O |
| 21 | 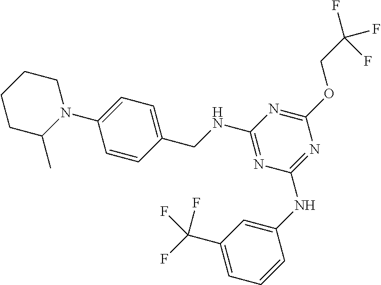 | N-[4-(2-methylpiperidin-1- yl)benzyl]-6-(2,2,2-trifluoroethoxy)- N′-[3-(trifluoromethyl)phenyl]-1,3,5- triazine-2,4-diamine | 540.5 | C25H26F6N6O |
| 22 | 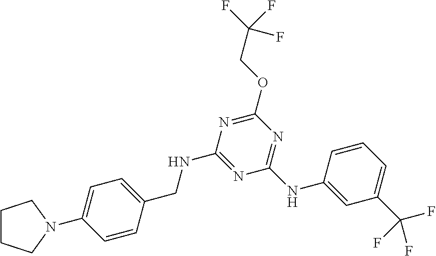 | N-(4-pyrrolidin-1-ylbenzyl)-6-(2,2,2- trifluoroethoxy)-N′-[3- (trifluoromethyl)phenyl]-1,3,5- triazine-2,4-diamine | 512.5 | C23H22F6N6O |
| 23 | 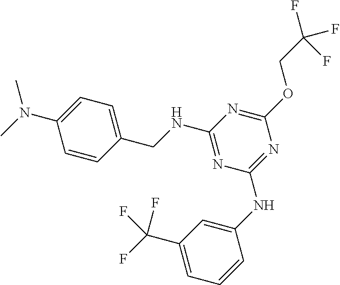 | N-[4-(dimethylamino)benzyl]-6- (2,2,2-trifluoroethoxy)-N′-[3- (trifluoromethyl)phenyl]-1,3,5- triazine-2,4-diamine | 486.4 | C21H20F6N6O |
| 24 | 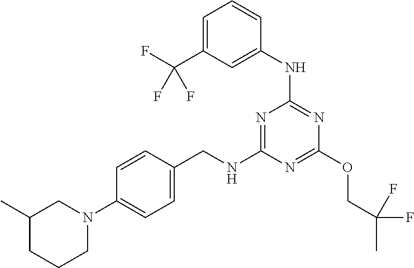 | N-[4-(3-methylpiperidin-1- yl)benzyl]-6-(2,2,2-trifluoroethoxy)- N′-[3-(trifluoromethyl)phenyl]-1,3,5- triazine-2,4-diamine | 540.5 | C25H26F6N6O |
| 25 | 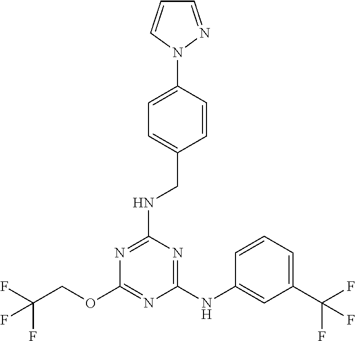 | N-[4-(1H-pyrazol-1-yl)benzyl]-6- (2,2,2-trifluoroethoxy)-N′-[3- (trifluoromethyl)phenyl]-1,3,5- triazine-2,4-diamine | 509.4 | C22H17F6N7O |
| 26 | 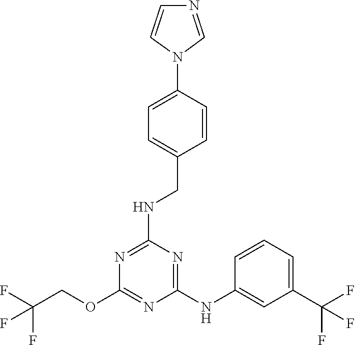 | N-[4-(1H-imidazol-1-yl)benzyl]-6- (2,2,2-trifluoroethoxy)-N′-[3- (trifluoromethyl)phenyl]-1,3,5- triazine-2,4-diamine | 509.4 | C22H17F6N7O |
| 27 | 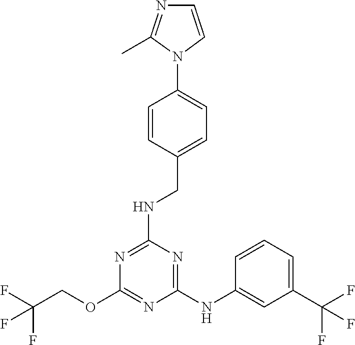 | N-[4-(2-methyl-1H-imidazol-1- yl)benzyl]-6-(2,2,2-trifluoroethoxy)- N′-[3-(trifluoromethyl)-phenyl]- 1,3,5-triazine-2,4-diamine | 523.4 | C23H19F6N7O |
| 28 | 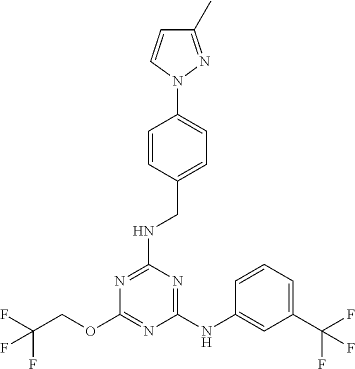 | N-[4-(3-methyl-1H-pyrazol-1- [symbol] l)benzyl]-6-(2,2,2-trifluoroethoxy)-N′- 3-(trifluoromethyl)phenyl]-1,3,5- [symbol] razine-2,4-diamine | 523.4 | C23H19F6N7O |
| 29 | 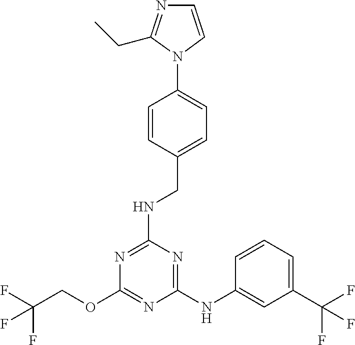 | N-[4-(2-ethyl-1H-imidazol-1- yl)benzyl]-6-(2,2,2-trifluoroethoxy)- N′-[3-(trifluoromethyl)phenyl]-1,3,5- triazine-2,4-diamine | 537.5 | C24H21F6N7O |
| 30 | 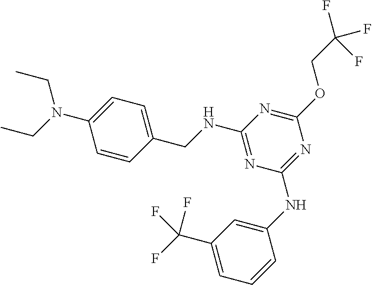 | N-[4-(diethylamino)benzyl]-6-(2,2,2- trifluoroethoxy)-N′-[3- (trifluoromethyl)phenyl]-1,3,5-triazine- 2,4-diamine | 514.5 | C23H24F6N6O |

[symbol]

indicates data missing or illegible when filed

### Procedures and Analytical Data for Table 22.

Entries 1, 3, 5 to 19, and 30 were prepared by the methods for Library 22a. Entries 2, 4 and 20 to 29 were prepared by the methods for Library 22b.

### 1. N-[4-(morpholin-4-yl)benzyl]-6-(2,2,2-trifluoroethoxy)-N′-[3-(trifluoromethyl)phenyl]-1,3,5-triazine-2,4-diamine

LCMS: M+1=528.4; 1H NMR (DMSO-d6, 90° C., ppm): d=3.08 q (4H), 3.78 t (4H), 4.52 s (2H), 4.86 q (2H), 6.84 d (2H), 7.40 m (5H), 7.96 s (1H), 8.16 s (1H), 9.40 s (1H).

### 2. N-[4-(3,5-dimethyl-1H-pyrazol-1-yl)benzyl]-6-(2,2,2-trifluoroethoxy)-N′-[3-(trifluoromethyl)phenyl]-1,3,5-triazine-2,4-diamine

LCMS: M+1=538.6 NMR 1H, DMSO-d6 δ, ppm: 2.27 d (6H); 4.52 d (2H); 4.92 m (2H); 6.00 s (1H); 7.30-7.55 m (6H); 7.80-8.20 m (3H); 9.62 bs (1H).

### 3. N-[4-(piperidin-1-yl)benzyl]-6-(2,2,2-trifluoroethoxy)-N′-[3-(trifluoromethyl)phenyl]-1,3,5-triazine-2,4-diamine

LCMS: M+1=526.4.

### 4. N-[4-(1H-pyrrol-1-yl)benzyl]-6-(2,2,2-trifluoroethoxy)-N′-[3-(trifluoromethyl)phenyl]-1,3,5-triazine-2,4-diamine

1H NMR (DMSO-d6, 90° C., ppm): d=4.59 d (2H, CH2), 4.94 q (2H, CH2), 6.25 t (2H, Ar), 7.24 s (2H, Ar), 7.31 d (1H, Ar), 7.45 m (5H, Ar), 7.96 s (2H, Ar), 8.15 s (1H, Ar), 9.62 s (1H, NH).

LC-MS [M+1]: calc'd: 508.4; obs'd: 509.8.

### 5. N-[4-{[4-(2,2,2-trifluoroethoxy)-6-{[3-(trifluoromethyl)phenyl]amino}-1,3,5-triazin-2-yl]amino}methyl)phenyl]furan-2-carboxamide

LCMS: M+1=515.1 NMR 1H, DMSO-d6 δ, ppm: 1.14 t (3H); 2.10 m (2H); 4.55 d (2H); 4.92 m (2H); 7.25 t (3H); 7.40-7.60 m (3H); 7.80-8.20 m (3H); 9.40 bs (1H); 9.60 s (1H).

### 6. N-(4-{[(4-(2,2,2-trifluoroethoxy)-6-{[3-(trifluoromethyl)phenyl]amino}-1,3,5-triazin-2-yl)amino]methyl}phenyl)isonicotinamide

LCMS: M+1=564.1 NMR 1H, DMSO-d6 δ, ppm: 4.55 d (2H); 4.96 m (2H); 7.26 t (3H); 7.52 t (1H); 7.70 d (2H); 7.88 m (4H); 8.20 m (1H); 8.78 d (2H); 9.58 bs (1H); 10.16 s (1H).

### 7. 1-ethyl-N-(4-{[(4-(2,2,2-trifluoroethoxy)-6-{[3-(trifluoromethyl)phenyl]amino}-1,3,5-triazin-2-yl)amino]methyl}phenyl)-1H-pyrazole-3-carboxamide

LCMS: M+1=581.1 NMR 1H, DMSO-d6 δ, ppm: 1.50 t (3H); 4.27 m (2H); 4.55 d (2H); 4.92 m (2H); 6.70 d (1H); 7.25 m (3H); 7.45 t (1H); 7.80-8.20 m (6H); 9.25 s (1H). 9.58 bs (1H).

### 8. 2-chloro-N-(4-{[(4-(2,2,2-trifluoroethoxy)-6-{[3-(trifluoromethyl)phenyl]amino}-1,3,5-triazin-2-yl)amino]methyl}phenyl)benzamide

LCMS: M+1=597.1 NMR 1H, DMSO-d6 δ, ppm: 4.55 d (2H); 4.92 m (2H); 7.20-7.30 m (3H); 7.35-7.55 m (5H); 7.62 d (2H); 7.80-8.20 m (3H); 9.58 bs (1H); 10.00 s (1H).

### 9. 4-methyl-N-(4-{[(4-(2,2,2-trifluoroethoxy)-6-{[3-(trifluoromethyl)phenyl]amino}-1,3,5-triazin-2-yl)amino]methyl}phenyl)benzamide

LCMS: M+1=577.6 NMR 1H, DMSO-d6 δ, ppm: 2.42 s (3H); 4.55 d (2H); 4.92 m (2H); 7.28 m (5H); 7.44 t (1H); 7.69 d (2H); 7.78 t (1H); 7.80-8.20 m (4H); 9.58 bs (1H); 9.78 s (1H).

### 10. 2-methyl-N-(4-{[(4-(2,2,2-trifluoroethoxy)-6-{[3-(trifluoromethyl)phenyl]amino}-1,3,5-triazin-2-yl)amino]methyl}phenyl)benzamide

LCMS: M+1=577.6 NMR 1H, DMSO-d6 δ, ppm: 2.42 s (3H); 4.55 d (2H); 4.92 m (2H); 7.20-7.36 m (6H); 7.44 t (2H); 7.69 d (2H); 7.80-8.20 m (3H); 9.58 bs (1H); 9.82 s (1H).

### 11. 3-methyl-N-(4-{[(4-(2,2,2-trifluoroethoxy)-6-{[3-(trifluoromethyl)phenyl]amino}-1,3,5-triazin-2-yl)amino]methyl}phenyl)benzamide

LCMS: M+1=577.6 NMR 1H, DMSO-d6 δ, ppm: 2.42 s (3H); 4.55 d (2H); 4.92 m (2H); 7.25-7.36 m (5H); 7.44 t (1H); 7.66-7.76 m (5H); 7.80-8.20 m (2H); 9.58 bs (1H); 9.81 s (1H).

### 12. 2-methyl-N-(4-{[(4-(2,2,2-trifluoroethoxy)-6-{[3-(trifluoromethyl)phenyl]amino}-1,3,5-triazin-2-yl)amino]methyl}phenyl)propanamide

LCMS: M+1=529.1 NMR 1H, DMSO-d6 δ, ppm: 1.12 d (6H); 2.55 m (1H); 4.55 d (2H); 4.92 m (2H); 7.25 t (3H); 7.40-7.60 m (3H); 7.80-8.20 m, (3H); 9.30 bs (1H); 9.60 s (1H).

### 13. N-(4-{[(4-(2,2,2-trifluoroethoxy)-6-{[3-(trifluoromethyl)phenyl]amino}-1,3,5-triazin-2-yl)amino]methyl}phenyl)benzamide

LCMS: M+1=563.6; 1H NMR, DMSO-d6 δ, ppm: 4.55 d (2H); 4.92 m (2H); 7.22-7.32 m (3H); 7.42-7.56 m (4H); 7.72 d (2H); 7.78 t (1H); 7.96 d (3H); 8.20 bm (1H); 9.58 bs (1H); 9.85 s (1H).

### 14. 3-chloro-N-(4-{[(4-(2,2,2-trifluoroethoxy)-6-{[3-(trifluoromethyl)phenyl]amino}-1,3,5-triazin-2-yl)amino]methyl}phenyl)benzamide

LCMS: M+1=597.1 NMR 1H, DMSO-d6 δ, ppm: 4.55 d (2H); 4.92 m (2H); 7.20-7.30 m (3H); 7.40-7.60 m (3H); 7.70 d (2H); 7.80 t (1H); 7.95 m (3H); 8.20 bs (1H); 9.58 bs (1H); 10.00 s (1H).

### 15. 4-chloro-N-(4-{[(4-(2,2,2-trifluoroethoxy)-6-{[3-(trifluoromethyl)phenyl]amino}-1,3,5-triazin-2-yl)amino]methyl}phenyl)benzamide

LCMS: M+1=597.1; 1H NMR, DMSO-d6 δ, ppm: 4.55 d (2H); 4.92 m (2H); 7.20-7.30 m (3H); 7.35-7.55 m (3H); 7.62 d (2H); 7.80-8.20 m (5H); 9.58 bs (1H); 10.00 s (1H).

### 16. N-(4-{[(4-(2,2,2-trifluoroethoxy)-6-{[3-(trifluoromethyl)phenyl]amino}-1,3,5-triazin-2-yl)amino]methyl}phenyl)-2-furamide

LCMS: M+1=553.1; 1H NMR, DMSO-d6 δ, ppm: 4.55 d (2H); 4.92 m (2H); 6.65 t (1H); 7.20-7.30 m (3H); 7.50 t (1H); 7.75 d (2H); 7.80-8.20 m (4H); 9.38 bs (1H); 9.78 (1H).

### 17. N-(4-{[(4-(2,2,2-trifluoroethoxy)-6-{[3-(trifluoromethyl)phenyl]amino}-1,3,5-triazin-2-yl)amino]methyl}phenyl)ethanesulfonamide

LCMS: M+1=551.1; 1H NMR, DMSO-d6 δ, ppm: 1.22 t (3H); 3.10 m (2H); 4.55 d (2H); 4.92 m (2H); 7.17 d (2H); 7.30 t (3H); 7.50 t (1H); 7.80-8.20 m (3H); 9.40 s (1H); 9.60 bs (1H).

### 18. N-(4-{[(4-(2,2,2-trifluoroethoxy)-6-{[3-(trifluoromethyl)phenyl]amino}-1,3,5-triazin-2-yl)amino]methyl}phenyl)methanesulfonamide

LCMS: M+1=537.1; 1H NMR, DMSO-d6 δ, ppm: 2.95 s (3H); 4.55 d (2H); 4.92 m (2H); 7.17 d (2H); 7.27 d (2H); 7.50 t (1H); 7.80-8.20 m (3H); 9.35 s (1H); 9.60 bs (1H).

### 19. N-(4-{[(4-(2,2,2-trifluoroethoxy)-6-{[3-(trifluoromethyl)phenyl]amino}-1,3,5-triazin-2-yl)amino]methyl}phenyl)propane-2-sulfonamide

LCMS: M+1=565.1; 1H NMR, DMSO-d6 δ, ppm: 1.25 d (6H); 3.21 m (1H); 4.55 d (2H); 4.92 m (2H); 7.15-7.50 m (6H); 7.80-8.20 m (3H); 9.35 s (1H); 9.60 bs (1H).

### 20. N-[4-(4-methylpiperidin-1-yl)benzyl]-6-(2,2,2-trifluoroethoxy)-N′-[3-(trifluoromethyl)phenyl]-1,3,5-triazine-2,4-diamine

1H NMR (DMSO-d6, 90° C., ppm): d=0.94 d (3H, CH3), 1.26 q (2H, CH2), 1.52 m (1H, CH), 1.68 q (2H, CH2), 2.68 t (2H, CH2), 3.58 t (2H, CH2), 4.46 d (2H, CH2), 4.92 q (2H, CH2), 6.84 d (2H, Ar), 7.16 d (2H, Ar), 7.30 d (1H, Ar), 7.48 t (1H, Ar), 7.78 t (1H, Ar), 7.94 t (1H, Ar), 8.16 d (1H, Ar), 9.58 s (1H, Ar). LC-MS [M+1]: calc'd: 540.5; obs'd: 541.4.

### 21. N-[4-(2-methylpiperidin-1-yl)benzyl]-6-(2,2,2-trifluoroethoxy)-N′-[3-(trifluoromethyl)phenyl]-1,3,5-triazine-2,4-diamine

1H NMR (DMSO-d6, 90° C., ppm): d=0.94 d (3H, CH3), 1.52 q (2H, CH2), 1.68 m (2H, CH2), 2.90 t (1H, CH2), 3.00 s (2H, CH2), 3.18 d (1H, CH2), 3.84 m (1H, CH), 4.46 d (2H, CH2), 4.94 q (2H, CH2), 6.84 d (2H, Ar), 7.16 d (2H, Ar), 7.30 d (1H, Ar), 7.48 t (1H, Ar), 7.88 t (2H, Ar), 8.16 t (1H, Ar), 9.64 s (1H, Ar). LC-MS [M+1]: calc'd: 540.5; obs'd: 541.5.

### 22. N-(4-pyrrolidin-1-ylbenzyl)-6-(2,2,2-trifluoroethoxy)-N′-[3-(trifluoromethyl)phenyl]-1,3,5-triazine-2,4-diamine

1H NMR (DMSO-d6, 90° C., ppm): d=1.96 m (4H, 2CH2), 3.22 t (4H, 2CH2), 4.44 d (2H, CH2), 4.94 q (2H, CH2), 6.50 d (2H, Ar), 7.16 d (2H, Ar), 7.30 d (1H, Ar), 7.50 t (1H, Ar), 7.74 t (1H, Ar), 7.96 t (1H, Ar), 8.18 d (1H, Ar), 9.58 s (1H, Ar). LC-MS [M+1]: calc'd: 512.5; obs'd: 513.7.

### 23. N-[4-(dimethylamino)benzyl]-6-(2,2,2-trifluoroethoxy)-N′-[3-(trifluoromethyl)phenyl]-1,3,5-triazine-2,4-diamine

1H NMR (DMSO-d6, 90° C., ppm): d=2.86 m (6H, 2CH3), 4.44 d (2H, CH2), 4.94 q (2H, CH2), 6.68 d (2H, Ar), 7.16 d (2H, Ar), 7.30 d (1H, Ar), 7.50 t (1H, Ar), 7.80 t (1H, Ar), 7.94 m (1H, Ar), 8.18 s (1H, Ar), 9.64 s (1H, Ar). LC-MS [M+1]: calc'd: 486.4; obs'd: 487.5.

### 24. N-[4-(3-methylpiperidin-1-yl)benzyl]-6-(2,2,2-trifluoroethoxy)-N′-[3-(trifluoromethyl)phenyl]-1,3,5-triazine-2,4-diamine

1H NMR (DMSO-d6, 90° C., ppm): d=0.90 d (3H, CH3), 1.04 q (1H, CH), 1.68 m (4H, 2CH2), 2.32 t (1H, CH2), 2.64 t (1H, CH2), 3.50 d (2H, CH2), 4.46 d (2H, CH2), 4.84 q (2H, CH2), 6.86 d (2H, Ar), 7.16 d (2H, Ar), 7.30 d (1H, Ar), 7.48 t (1H, Ar), 7.88 t (2H, Ar), 8.16 s (1H, Ar), 9.64 s (1H, Ar). LC-MS [M+1]: calc'd: 540.5; obs'd: 541.5.

### 25. N-[4-(1H-pyrazol-1-yl)benzyl]-6-(2,2,2-trifluoroethoxy)-N′-[3-(trifluoromethyl)phenyl]-1,3,5-triazine-2,4-diamine

LCMS: M+1=510.6; 1H NMR, DMSO-d6 δ, ppm: 4.52 d (2H); 4.92 m (2H); 6.47 s (1H); 7.25-8.25 m (10H); 9.61 bs (1H).

### 26. N-[4-(1H-imidazol-1-yl)benzyl]-6-(2,2,2-trifluoroethoxy)-N′-[3-(trifluoromethyl)phenyl]-1,3,5-triazine-2,4-diamine

LCMS: M+1=510.6; 1H NMR, DMSO-d6 δ, ppm: 4.52 d (2H); 4.92 m (2H); 7.08 s (1H); 7.25-7.60 m (7H); 7.90-8.20 m (4H); 9.58 bs (1H).

### 27. N-[4-(2-methyl-1H-imidazol-1-yl)benzyl]-6-(2,2,2-trifluoroethoxy)-N′-[3-(trifluoromethyl)phenyl]-1,3,5-triazine-2,4-diamine

LCMS: M+1=525.6; 1H NMR, DMSO-d6 δ, ppm: 2.27 s (3H); 4.52 d (2H); 4.92 m (2H); 6.84 s (1H); 7.06 s (1H); 7.25-7.30 m (3H); 7.45-7.55 m (3H); 7.92 t (2H); 8.12 s (1H); 9.62 bs (1H).

### 28. N-[4-(3-methyl-1H-pyrazol-1-yl)benzyl]-6-(2,2,2-trifluoroethoxy)-N′-[3-(trifluoromethyl)phenyl]-1,3,5-triazine-2,4-diamine

LCMS: M+1=525.6; 1H NMR, DMSO-d6 δ, ppm: 2.27 s (3H); 4.52 d (2H); 4.92 m (2H); 6.20 d (1H); 7.25 d (1H); 7.43 m (3H); 7.65 d (2H); 7.85 m (2H); 8.20 m (2H); 9.62 bs (1H).

### 29. N-[4-(2-ethyl-1H-imidazol-1-yl)benzyl]-6-(2,2,2-trifluoroethoxy)-N′-[3-(trifluoromethyl)phenyl]-1,3,5-triazine-2,4-diamine

LCMS: M+1=538.6; 1H NMR, DMSO-d6 δ, ppm: 1.13 t (3H); 2.58 m (2H); 4.52 d (2H); 4.92 m (2H); 6.90 s (1H); 7.10 s (1H); 7.25-7.55 m (6H); 7.45-7.55 m (3H); 7.92-8.20 m (3H); 9.62 bs (1H).

### 30. N-[4-(diethylamino)benzyl]-6-(2,2,2-trifluoroethoxy)-N′-[3-(trifluoromethyl)phenyl]-1,3,5-triazine-2,4-diamine

1H NMR (DMSO-d6, 90° C., ppm): d=1.08 t (6H, 2CH3), 3.30 q (4H, 2CH2), 4.42 d (2H, CH2), 4.94 q (2H, CH2), 6.64 d (2H, Ar), 7.14 d (2H, Ar), 7.30 d (1H, Ar), 7.48 t (1H, Ar), 7.82 t (1H, Ar), 7.94 t (1H, Ar), 8.18 d (1H, Ar), 9.64 s (1H, Ar). LC-MS [M+1]: calc'd: 514.5; obs'd: 515.6.

### Generic Synthesis of Intermediates and Final Compounds for Table 23

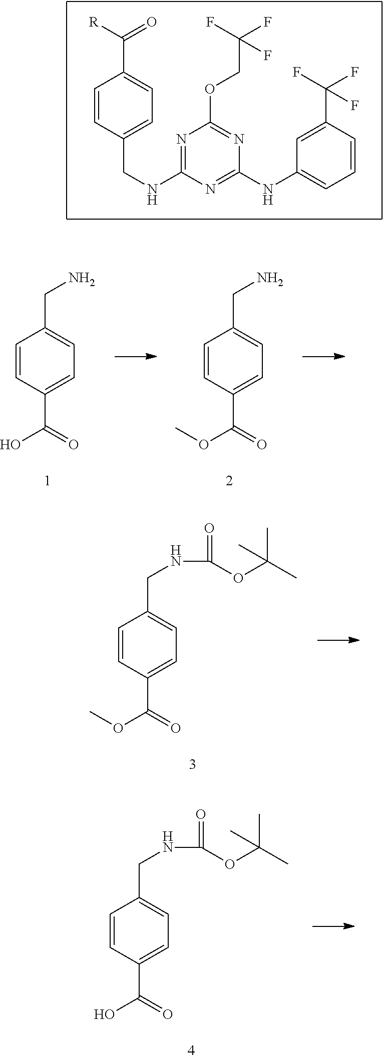

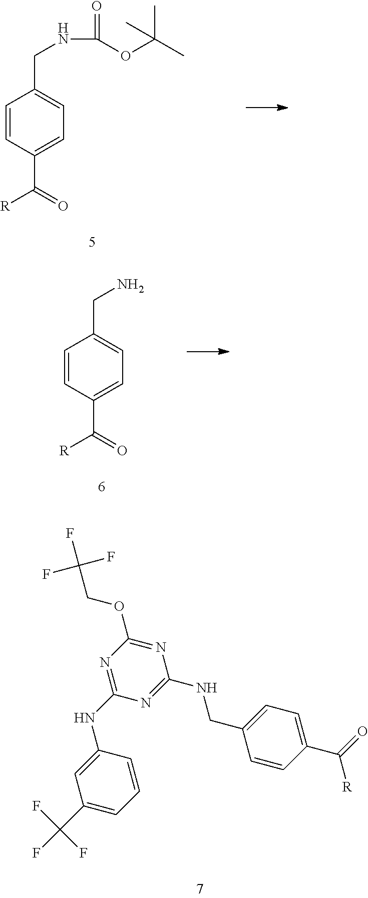

Intermediate 2 was obtained according to the following procedure: To a stirred solution of 70 g of intermediate 1 in 700 ml of methanol, 76 ml of SOCl₂ were added dropwise and the reaction mixture was stirred at reflux for 14 hours. At this time, the solvent was removed and the product was used in the next step without further purification.

Intermediate 3 was obtained according to the following procedure: BOC-anhydride (46 g) was added dropwise to a stirred solution of crude ester 2 in CH₂Cl₂. The reaction mixture was stirred at r.t. for 7 hours, concentrated in vacuo, and the residue washed with hexane providing 3 as white solid. Yield 56% (26 g).

Intermediate 4 was obtained according to the following procedure: KOH (4 g) was added portion-wise to a stirred suspension of N-Boc-ester 3 (14 g) in water. The reaction mixture was stirred at 50° C. for 7 h, then acidified with HCl, filtered and washed with water to provide intermediate 4 as white solid, 85% yield.

Intermediate 6 (via 5) was obtained according to the following procedure: Intermediate 4 (0.01 mol) was combined with CDI (0.011 mol) in 1,4-dioxane and stirred for 2 h at 50° C. Corresponding amine (0.01 mol) was added to the reaction mixture. The mixture was stirred at 50° C. for 7 hours, diluted with water, extracted with ethyl acetate, dried over sodium sulphate, concentrated in vacuo and treated with saturated HCl-dioxane for 5 h at rt. The solvent was removed under reduced pressure to provide 6 as a hydrochloride salt.

Compound 7 was obtained according to the following procedure: A mixture of compound 6 (1.53 g) and monochlorotriazine (2 g) in dioxane was treated with triethylamine (0.8 g) at 50° C. for 5 hours. The solvent was removed under reduced pressure and the residue was washed with water, filtered, dried, and purified using column chromatography with CH₂Cl₂ as eluent to give compound 7 as white solid.

TABLE 23

| En | Structure | IUPAC Name | MW | Formula |
|---|---|---|---|---|
| 1 | 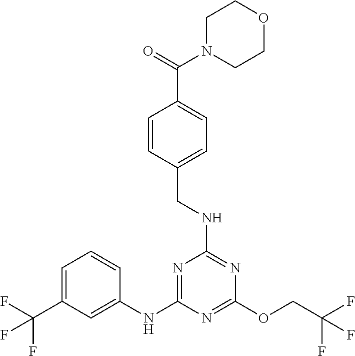 | morpholin-4-yl[4-({[4-(2,2,2- trifluoroethoxy)-6-{[3- (trifluoromethyl)phenyl]amino}- 1,3,5-triazin-2- yl]amino}methyl)phenyl]methanone | 556.5 | C₂₄H₂₂F₆N₆O₃ |
| 2 | 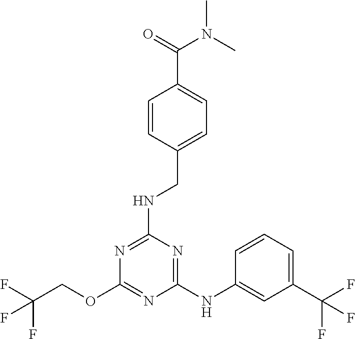 | N,N-dimethyl-4-({[4-(2,2,2- trifluoroethoxy)-6-{[3- (trifluoromethyl)phenyl]amino}- 1,3,5-triazin-2- yl]amino}methyl)benzamide | 514.4 | C₂₂H₂₀F₆N₆O₂ |
| 3 | 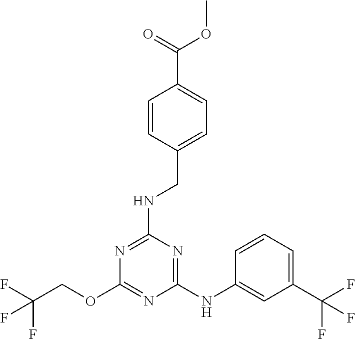 | methyl 4-({[4-(2,2,2-trifluoroethoxy)- 6-{[3-(trifluoromethyl)phenyl]amino}- 1,3,5-triazin-2-yl]amino}methyl)benzoate | 501.4 | C₂₁H₁₇F₆N₅O₃ |
| 4 | 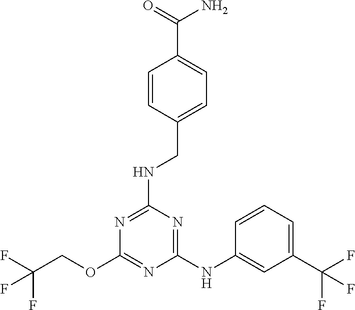 | 4-({[4-(2,2,2-trifluoroethoxy)-6-{[3- (trifluoromethyl)phenyl]amino}-1,3,5- triazin-2-yl]amino}methyl)benzamide | 486.4 | C₂₀H₁₆F₆N₆O₂ |
| 5 | 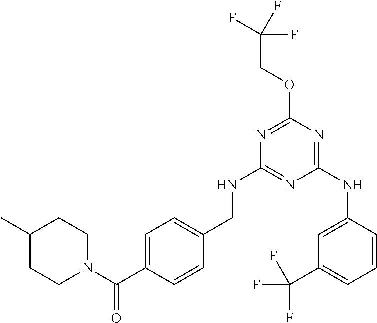 | N-{4-[(4-methylpiperidin- 1-yl)carbonyl]benzyl}- 6-(2,2,2-trifluoroethoxy)-N′-[3- (trifluoromethyl)phenyl]- 1,3,5-triazine-2,4-diamine | 568.5 | C₂₆H₂₆F₆N₆O₂ |
| 6 | 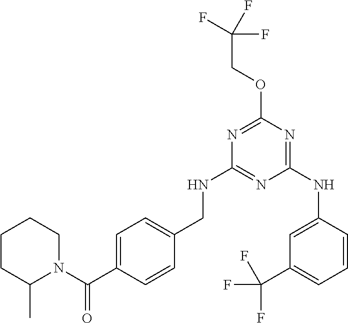 | N-[4-(4-benzylpiperazin-1- yl)benzyl]-6-(2,2,2- trifluoroethoxy)-N′-[3- (trifluoromethyl)phenyl]- 1,3,5-triazine-2,4-diamine | 568.5 | C₂₆H₂₆F₆N₆O₂ |
| 7 | 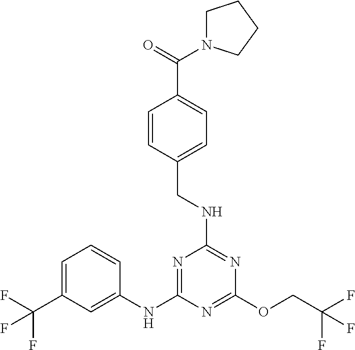 | pyrrolidin-1-yl(4-((4-(2,2,2- trifuoroethoxy)-6- (3-(trifluoromethyl)phenylamino)- 1,3,5-triazin- 2-ylamino)methyl)phenyl)methanone | 540.5 | C₂₄H₂₂F₆N₆O₂ |
| 8 | 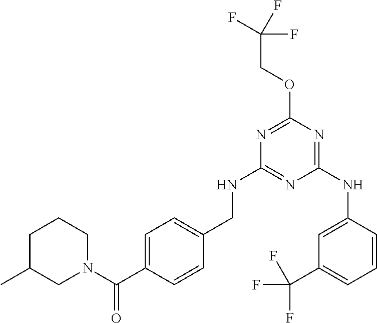 | N-{4-[(3-methylpiperidin-1- yl)carbonyl]benzyl}- 6-(2,2,2-trifluoroethoxy)-N′-[3- (trifluoromethyl)phenyl]-1,3,5-triazine- 2,4-diamine | 568.5 | C₂₆ H₂₆F₆N₆O₂ |
| 9 | 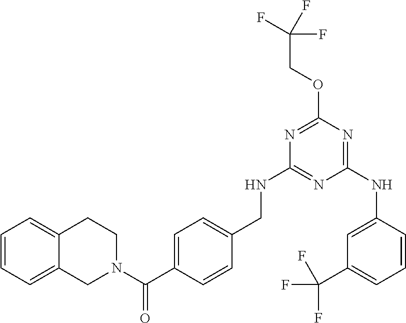 | N-[4-(3,4-dihydroisoquinolin-2(1H)- ylcarbonyl)benzyl]-6-(2,2,2- trifluoroethoxy)-N′-[3- (trifluoromethyl)phenyl]-1,3,5-triazine- 2,4-diamine | 602.5 | C₂₉H₂₄F₆N₆ |
| 10 | 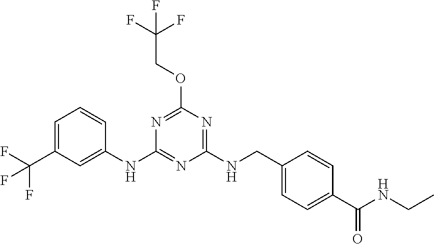 | N-ethyl-4-({[4-(2,2,2-trifluoroethoxy)-6- {[3-(trifluoromethyl)phenyl]amino]-1,3,5- triazin-2-yl]amino}methyl)benzamide | 514.4 | C₂₁H₂₀F₆N₆O₃S₂ |
| 11 | 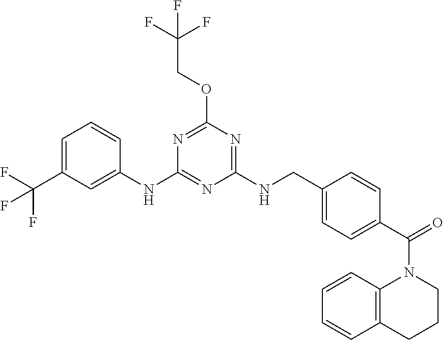 | N-ethyl-2-{4-[4-({[4-(2,2,2- trifluoroethoxy)-6-{[3- (trifluoromethyl)phenyl]amino}-1,3,5- triazin-2- yl]amino}methyl)phenyl]piperazin-1- yl}acetamide | 602.5 | C₂₉H₂₅F₆N₆O₂ |

### Procedures and Analytical Data for Table 23.

All compounds were prepared by the above procedures.

### 1. morpholin-4-yl[4-({[4-(2,2,2-trifluoroethoxy)-6-{[3-(trifluoromethyl)phenyl]amino}-1,3,5-triazin-2-yl]amino}methyl)phenyl]methanone

LCMS: M+1=556.4; 1H NMR (DMSO-d6, 90° C., ppm): d=3.5 (8H, m); 4.6 (2H, d); 4.9 (2H. q); 7.4 (6H, m); 8.0 (3H, m); 9.65 (1H, s).

### 2. N,N-dimethyl-4-{[4-(2,2,2-trifluoroethoxy)-6-{[3-(trifluoromethyl)phenyl]amino}-1,3,5-triazin-2-yl]amino}methyl)benzamide

LCMS: M+1=514.4; 1H NMR (DMSO-d6, 90° C., ppm): d=3.0 (6H, m); 4.6 (2H, d); 4.95 (2H, q); 7.4 (6H, m); 7.95 (2H, m); 8.1 (1H, s); 9.6 (1H, s).

### 3. methyl 4-{[4-(2,2,2-trifluoroethoxy)-6-{[3-(trifluoromethyl)phenyl]amino}-1,3,5-triazin-2-yl]amino}methyl)benzoate

1H NMR (DMSO-d6, 90° C., ppm): d=3.85 s (3H, CH3), 4.64 d (2H, CH2), 4.93 q (2H, CH2), 7.30 d (1H, Ar), 7.46 d (3H, Ar), 7.92 d (3H, Ar), 8.06 t (1H, Ar), 8.13 (1H, Ar), 9.65 s (1H, NH); LC-MS [M+1]: calc'd: 501.3; obs'd: 502.2.

### 4. 4-{[4-(2,2,2-trifluoroethoxy)-6-{[3-(trifluoromethyl)phenyl]amino}-1,3,5-triazin-2-yl]amino}methyl)benzamide

1H NMR (DMSO-d6, 90° C., ppm): d=4.60 s (2H, CH2), 4.94 d (2H, CH2), 7.22 s (1H, Ar), 7.30 d (2H, Ar), 7.40 d (2H, Ar), 7.48 t (1H, Ar), 7.82 d (2H, Ar), 7.91 s 1H, Ar), 8.02 s (1H, Ar), 8.15 s (1H, Ar), 9.64 s (1H, NH). LC-MS [M+1]: calc'd: 486.3; obs'd: 487.5.

### 5. N-{4-[(4-methylpiperidin-1-yl)carbonyl]benzyl}-6-(2,2,2-trifluoroethoxy)-N′-[3-(trifluoromethyl)phenyl]-1,3,5-triazine-2,4-diamine

LCMS: M+1=569.6; 1H NMR, DMSO-d6 δ, ppm: 0.93 d (3H); 1.08 m (2H); 1.65 m (3H); 2.88 m (2H); 3.95 d (2H); 4.60 d (2H); 4.92 m (2H); 7.30 d (3H); 7.37 d (2H); 7.47 t (1H); 7.95 m (2H); 8.12 m (1H); 9.58 bs (1H).

### 6. N-[4-(4-benzylpiperazin-1-yl)benzyl]-6-(2,2,2-trifluoroethoxy)-N′-[3-(trifluoromethyl)phenyl]-1,3,5-triazine-2,4-diamine

LCMS: M+1=569.6; 1H NMR, DMSO-d6 δ, ppm: 1.10-1.60 m (9H); 3.85 d (1H); 4.35 s (1H); 4.52 d (2H); 4.92 m (2H); 7.25-7.55 m (6H); 7.80-8.20 m (3H); 9.35 bs (1H).

### 7. pyrrolidin-1-yl(4-((4-(2,2,2-trifluoroethoxy)-6-(3-(trifluoromethyl)phenylamino)-1,3,5-triazin-2-ylamino)methyl)phenyl)methanone

LCMS: M+1=541.6; 1H NMR, DMSO-d6 δ, ppm: 1.87 t (4H); 3.44 t (4H); 4.60 d (2H); 4.87 m (2H); 7.24 d (1H); 7.40 m (5H); 7.90-8.20 m (3H); 9.58 bs (1H).

### 8. N-{4-[(3-methylpiperidin-1-yl)carbonyl]benzyl}-6-(2,2,2-trifluoroethoxy)-N′-[3-(trifluoromethyl)phenyl]-1,3,5-triazine-2,4-diamine

LCMS: M+1=569.6; 1H NMR, DMSO-d6 δ, ppm: 0.83 d (3H); 1.14 m (1′-1); 1.38 m (1H); 1.64 m (2H); 1.80 m (1H); 2.62 t (1H); 2.92 t (1H); 3.85 bs (2H); 4.52 d (2H); 4.92 m (2H); 7.25-7.55 m (6H); 7.80-8.20 m (3H); 9.68 bs (1H).

### 9. N-[4-(3,4-dihydroisoquinolin-2(1H)-ylcarbonyl)benzyl]-6-(2,2,2-trifluoroethoxy)-N′-[3-(trifluoromethyl)phenyl]-1,3,5-triazine-2,4-diamine

LCMS: M+1=603.6; 1H NMR, DMSO-d6 δ, ppm: 2.85 t (2H); 3.68 t (2H); 4.62 t (4H); 4.92 m (2H); 7.08 m (1H); 7.17 d (3H); 7.23-7.43 m (6H); 7.80-8.20 m (3H); 9.60 bs (1H).

### 10. N-ethyl-4-{[4-(2,2,2-trifluoroethoxy)-6-{[3-(trifluoromethyl)phenyl]amino}-1,3,5-triazin-2-yl]amino}methyl)benzamide

LCMS: M+1=603.6; 1H NMR, DMSO-d6 δ, ppm: 2.85 t (2H); 3.68 t (2H); 4.62 t (4H); 4.92 m (2H); 7.08 m (1H); 7.17 d (3H); 7.23-7.43 m (6H); 7.80-8.20 m (3H); 9.60 bs (1H).

### 11. N-[4-(3,4-dihydroquinolin-1(2H)-ylcarbonyl)benzyl]-6-(2,2,2-trifluoroethoxy)-N′-[3-(trifluoromethyl)phenyl]-1,3,5-triazine-2,4-diamine

LCMS: M+1=603.6 NMR 1H, DMSO-d6 δ, ppm: 1.99 m (2H); 2.85 t (2H); 3.77 t (2H); 4.57 d (2H); 4.87 m (2H); 6.84 d (2H); 6.96 m (1H); 7.17 d (1H); 7.28 m (5H); 7.44 t (1H); 7.90-8.20 m (3H); 9.68 bs (1H).

### Generic Synthesis of Intermediates and Final Compounds for Table 24

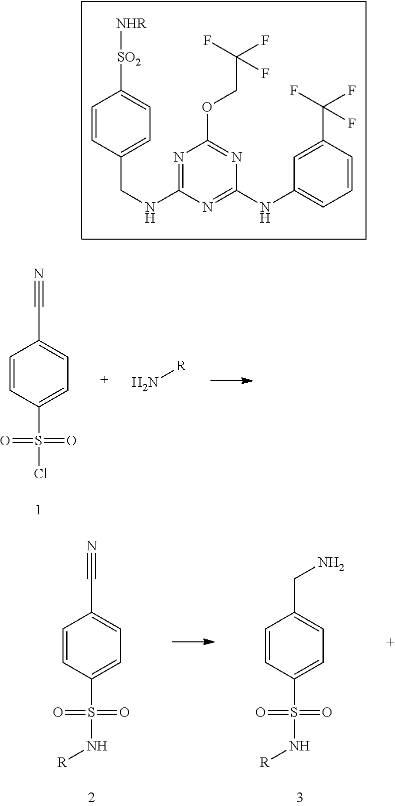

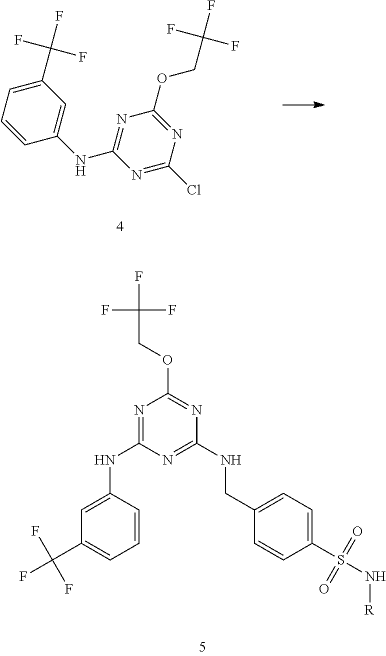

Intermediate 2 was synthesized according to the following procedure: A solution of 5 mmol of amine in 10 ml of dioxane was treated with sulphochloride 1 (5 mmol) and triethylamine (6 mmol). This mixture was stirred at reflux for 3 hours, then cooled and evaporated. The residue was treated with water. The precipitate thus formed was filtered and washed with water.

Intermediate 3 was synthesized according to the following procedure: Method A: Compound 2 (1 g.) was dissolved in THF (132 ml) and hydrogenated in the presence of LiAlH₄ (1 g) at 80° C. After the reaction was complete, a solution of aqueous KOH was added, and the reaction was filtered, and the solvent was removed under reduced pressure. Method B: Intermediate 2 (0.8 g, 3.2 mmol) was dissolved in ethanol (75 ml) and PtO₂.(75 mg) and Pd/C (10%. 100 mg) were added. The mixture was stirred for 2 h at 50° C. under 3 atm of H₂. LCMS analysis of the reaction mixture demonstrated the presence of target intermediate 4 in 70% conversion. The reaction mixture was filtered, and the volatile components were removed under reduced pressure. The solid obtained was washed with ether and dried.

Compound 5 was synthesized according to the following procedure: A mixture of 3 (1 mmol) and 4 (1 mmol) in dioxane was treated with triethylamine (1 mmol g) at 50° C. for 5 hours. The solvent was removed under reduced pressure and the residue was washed with water, filtered, dried, and purified by using column chromatography with CH₂Cl₂ as eluent to give 5 as white solid.

TABLE 24

| En | Structure | IUPAC Name | MW | Formula |
|---|---|---|---|---|
| 1 | 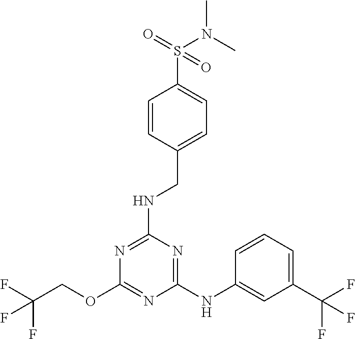 | N,N-dimethyl-4-({[4-(2,2,2- trifluoroethoxy)-6-{[3- (trifluoromethyl)phenyl]amino}- 1,3,5-triazin-2- yl]amino}methyl)benzenesulfonamide | 551.5 | C21H20F6N6O3S |
| 2 | 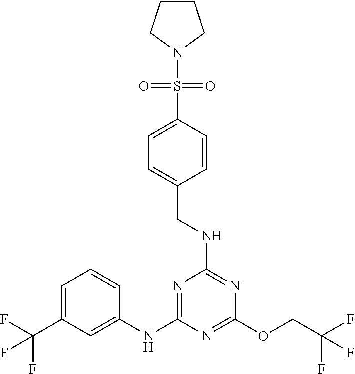 | N-[4-(pyrrolidin-1- ylsulfonyl)benzyl]-6-(2,2,2- trifluoroethoxy)-N′-[3- (trifluoromethyl)phenyl]-1,3,5- triazine-2,4-diamine | 577.5 | C23H22F6N6O3S |
| 3 | 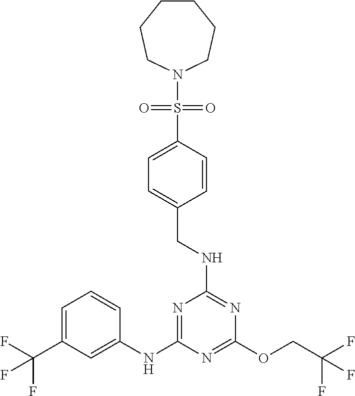 | N-[4-(azepan-1-ylsulfonyl)benzyl]- 6-(2,2,2-trifluoroethoxy)-N′-[3- (trifluoromethyl)phenyl]-1,3,5- triazine-2,4-diamine | 605.6 | C25H26F6N6O3S |
| 4 | 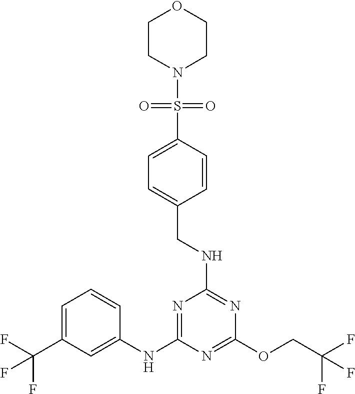 | N-[4-(morpholin-4- ylsulfonyl)benzyl]-6-(2,2,2- trifluoroethoxy)-N′-[3- (trifluoromethyl)phenyl]-1,3,5- triazine-2,4-diamine | 593.5 | C23H22F6N6O4S |
| 5 | 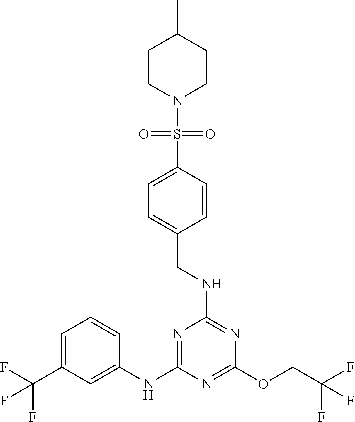 | N2-(4-(4-methylpiperidin-1- ylsulfonyl)benzyl)-6-(2,2,2- trifluoroethoxy)-N4-(3- (trifluoromethyl)-phenyl)-1,3,5- triazine-2,4-diamine | 605.6 | C25H26F6N6O3S |
| 6 | 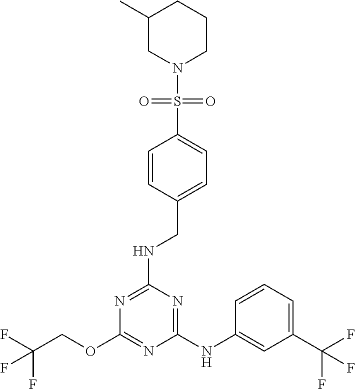 | N2-(4-(3-methylpiperidin-1- ylsulfonyl)benzyl)-6-(2,2,2- trifluoroethoxy)-N4-(3-(trifluoro- methyl)phenyl)-1,3,5-triazine-2,4- diamine | 605.6 | C25H26F6N6O3S |
| 7 | 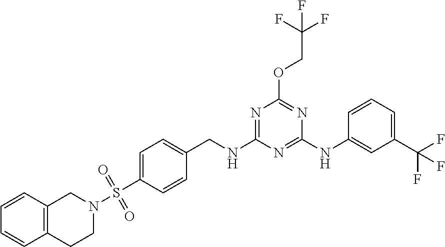 | N2-(4-(3,4-dihydroisoquinolin-2(1H)- ylsulfonyl)-benzyl)-6-(2,2,2- trifluoroethoxy)-N4-(3-(trifluoro- methyl)phenyl)-1,3,5-triazine-2,4- diamine | 639.6 | C28H24F6N6O3S |
| 8 | 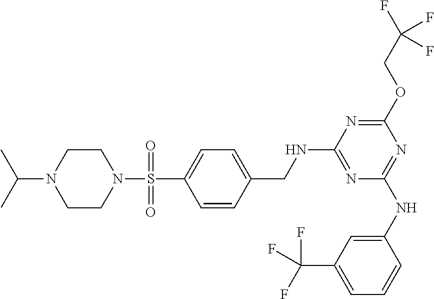 | N-{4-[(4-isopropylpiperazin-1- yl)sulfonyl]benzyl}-6-(2,2,2- trifluoroethoxy)-N′-[3- (trifluoromethyl)-phenyl]-1,3,5- triazine-2,4-diamine | 633.6 | C26H29F6N7O3S |

### Procedures and Analytical Data for Table 24.

Entries 2-4 were prepared by Method B for Intermediate 3. Entries 1 and 5-8 were prepared by Method A.

### 1. N,N-dimethyl-4-{[4-(2,2,2-trifluoroethoxy)-6-{[3-(trifluoromethyl)phenyl]amino}-1,3,5-triazin-2-yl]amino}methyl)benzenesulfonamide

LCMS: M+1=550.4; 1H NMR (DMSO-d6, 90° C., ppm): d=2.65 (6H, s); 4.6 (2H, d); 4.85 (2H, q); 7.3 (1H, d); 7.45 (1H, t); 7.55 (2H, d); 7.65 (2H, d); 8.0 (3H, m); 9.55 (1H, s).

### 2. N-[4-(pyrrolidin-1-ylsulfonyl)benzyl]-6-(2,2,2-trifluoroethoxy)-N′-[3-(trifluoromethyl)phenyl]-1,3,5-triazine-2,4-diamine

LCMS: M+1=576.5; 1H NMR (DMSO-d6, 90° C., ppm): d=1.65 (4H, m); 3.15 (4H, t); 4.6 (2H, d); 4.95 (2H, q); 7.3 (1H, d); 7.5 (3H, m); 7.9 (5H, m); 9.6 (1H, s).

### 3. N-[4-(azepan-1-ylsulfonyl)benzyl]-6-(2,2,2-trifluoroethoxy)-N′-[3-(trifluoromethyl)phenyl]-1,3,5-triazine-2,4-diamine

LCMS: M+1=604.5; 1H NMR (DMSO-d6, 90 o° C., ppm): d=1.6 (8H, m); 3.2 (4H, t); 4.6 (2H, d); 4.95 (2H, q); 7.3 (1H, d); 7.5 (3H, m); 7.9 (5H, m); 9.6 (1H, s).

### 4. N-[4-(morpholin-4-ylsulfonyl)benzyl]-6-(2,2,2-trifluoroethoxy)-N′-[3-(trifluoromethyl)phenyl]-1,3,5-triazine-2,4-diamine

LCMS: M+1=592.5; 1H NMR (DMSO-d6, 90° C., ppm): d=2.8 (4H, t); 3.6 (4H, t); 4.6 (2H, d); 4.9 (2H, q); 7.3 (1H, d); 7.45 (1Ht); 7.6 (2H, d); 7.7 (2H, d); 8.0 (3H, m); 9.7 (1H, s).

### 5. N-2-(4-(4-methylpiperidin-1-ylsulfonyl)benzyl)-6-(2,2,2-trifluoroethoxy)-N-4-(3-(trifluoromethyl)phenyl)-1,3,5-triazine-2,4-diamine

1H NMR (DMSO-d6, 90° C., ppm): d=0.84 d (3H, CH3), 1.12 t (2H, CH2), 1.30 d (2H, CH), 1.62 t (2H, CH2), 2.32 t (2H, CH2), 3.58 m (1H, CH), 4.64 d (2H, CH2), 4.94 q (2H, CH2), 7.30 d (1H, Ar), 7.58 m (5H, Ar), 7.88 s (1H, Ar), 8.08 t (2H, Ar), 9.62 s (1H, Ar). LC-MS [M+1]: calc'd: 604.6; obs'd: 605.4.

### 6. N-2-(4-(3-methylpiperidin-1-ylsulfonyl)benzyl)-6-(2,2,2-trifluoroethoxy)-N-4-(3-(trifluoromethyl)phenyl)-1,3,5-triazine-2,4-diamine

1H NMR (DMSO-d6, 90° C., ppm): d=0.84 s (4H, 2CH2), 1.44 t (1H, CH), 1.64 s (3H, CH3), 2.06 t (1H, CH), 2.36 t (1H, CH), 3.46 t (2H, CH2), 4.66 d (2H, CH2), 4.94 q (2H, CH2), 7.30 t (1H, Ar), 7.50 m (3H, Ar), 7.68 d (2H, Ar), 7.88 t (2H, Ar), 9.62 s (1H, Ar). LC-MS [M+1]: calc'd: 604.6; obs'd: 605.7.

### 7. N-2-(4-(3,4-dihydroisoquinolin-2(1H)-ylsulfonyl)benzyl)-6-(2,2,2-trifluoroethoxy)-N-4-(3-(trifluoromethyl)phenyl)-1,3,5-triazine-2,4-diamine

1H NMR (DMSO-d6, 90° C., ppm): d=1.68 t (2H, CH2), 2.42 t (2H, CH2), 3.74 t (2H, CH2), 4.62 d (2H, CH2), 4.92 q (2H, CH2), 7.02 t (2H, Ar), 7.12 m (1H, Ar), 7.28 d (1H, Ar), 7.48 t (3H, Ar), 7.58 d (3H, Ar), 7.88 t (1H, Ar), 8.02 s (1H, Ar), 8.10 s (1H, Ar), 9.62 s (1H, Ar). LC-MS [M+1]: calc'd: 638.6; obs'd: 639.4.

### 8. N-{4-[(4-isopropylpiperazin-1-yl)sulfonyl]benzyl}-6-(2,2,2-trifluoroethoxy)-N′-[3-(trifluoromethyl)phenyl]-1,3,5-triazine-2,4-diamine

1H NMR (DMSO-d6, 90° C., ppm): d=0.92 d (6H, 2CH3), 2.46 t (4H, 2CH2), 2.62 m (1H, CH), 2.92 t (4H, 2CH2), 4.66 d (2H, CH2), 4.84 q (2H, CH2), 7.30 d (1H, Ar), 7.48 t (1H, Ar), 7.58 d (2H, Ar), 7.70 d (2H, Ar), 7.90 t (1H, Ar), 8.08 m (2H, Ar), 9.62 s (1H, Ar). LC-MS [M+1]: calc'd: 633.6; obs'd: 634.5.

### Generic Synthesis of Intermediates and Final Compounds for Table 25.

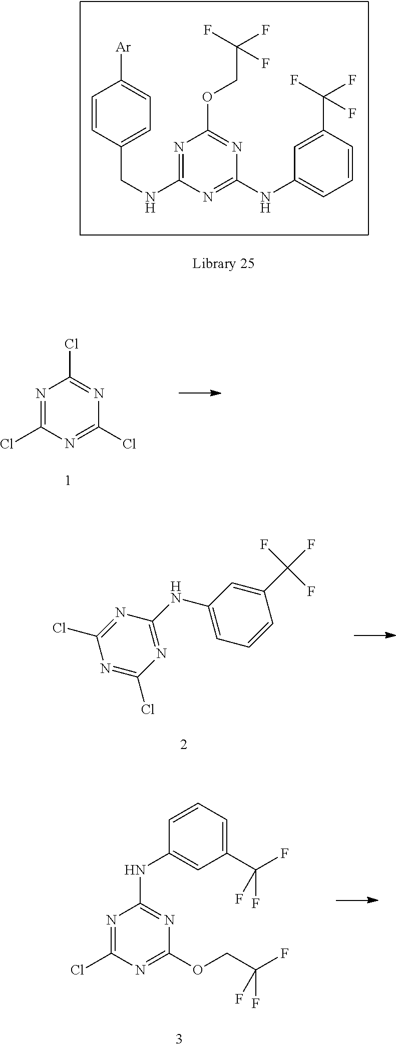

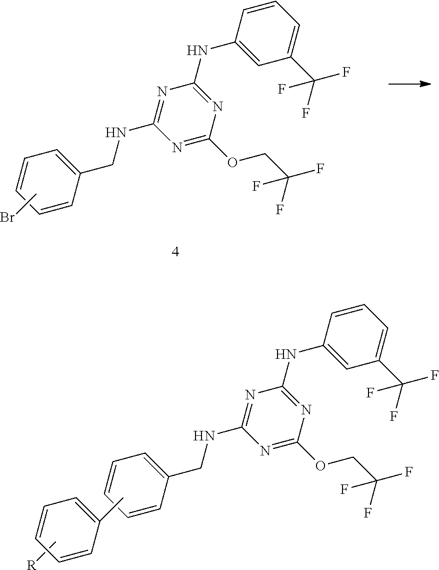

Preparation of 4-chloro-6-trifluoroethoxy-N-[3-(trifluoromethyl)phenyl]-1,3,5-triazine-2-amine 3. To a suspension of 0.500 g (2.781 mmol) 1, 0.225 g (2.781 mmol) of NaHCO₃ and 0.820 g (5.788 mmol) of Na₂SO₄ in 20 ml of anhydrous acetonitrile at −10° C. was added dropwise a solution of 3-(trifluoromethyl)aniline in 10 ml of dry acetonitrile, 0.450 g (2.781 mmol), over 2 h. After complete addition, the cooling bath was removed and the mixture was stirred at rt for 3 h. The resulting precipitate was filtered and the pale yellow solution of intermediate 2 (88% LCMS) was used in the next step without further purification.

To a solution of intermediate 2 in 30 ml anhydrous acetonitrile was added dropwise a cooled solution of potassium tert-butoxide (0.312 g, 2.781 mmol) in 2,2,2-trifluoro-1-ethanol (5 ml) over 2 h. After stirring this reaction mixture overnight at rt, the solid precipitate was filtered and washed with anhydrous acetonitrile (2×30 ml). The solvent was removed in vacuo to afford a yellow oil. To this oil was added (3×50 ml) anhydrous hexanes and the mixture was heated at reflux. After 1 min the hexane layer was decanted. The solvent was removed from the combined decanted fractions in vacuo to afford 3 as a white solid (0.595 g, 60%).

Preparation of N-(4-bromobenzyl)-6-(2,2,2-trifluoroethoxy)-N′-[3-(trifluoromethyl)phenyl]-1,3,5-triazine-2,4-diamine 4. Bromobenzylamine hydrochloride (2.8 mmol) was stirring for 1 hour in 1,4-dioxane (15 ml) with triethylamine (6 mmol) at 80° C. Chloride 3 (1 g, 2.67 mmol) was added and reaction mixture was stirred for 12 hours at 80° C. LCMS analysis of the reaction mixture after this time demonstrated presence of product (90%). The reaction mixture was diluted with water (200 mL) and extracted with ethyl acetate. The solvent was removed under reduced pressure, the precipitate filtered, and washed with ether to afford the desired product. P—Br: LCMS: M+1=523, ¹H NMR (DMSO, ppm) δ: 4.48 m (2H), 5.0 m (2H), 7.24 m (3H), 7.54 m (3H), 7.80 bs (1H), 8.18 d (1H), 8.4 bs (1H), 10.0 d (1H)

Preparation of N-(arylphenyl-4-ylmethyl)-6-(2,2,2-trifluoroethoxy)-N′-[3-(trifluoromethyl)-phenyl]-1,3,5-triazine-2,4-diamine 5 N-(4-bromobenzyl)-6-(2,2,2-trifluoroethoxy)-N′-[3-(tri-fluoromethyl)phenyl]-1,3,5-triazine-2,4-diamine 4 (100 mg, 0.19 mmol) was dissolved in 1,4-dioxane (5 ml). Boronic acid (0.2 mmol), Na₂CO₃ (5% water solution, 0.5 ml), and PdCl₂ (5 mol %) were then added. The reaction mixture was stirred for 12 hours at 80° C. LCMS analysis of the reaction mixture after this time demonstrated the presence of product (90%). The reaction mixture was diluted with water (200 mL) and extracted with ethyl acetate. The solvent was removed under reduced pressure to afford final compounds.

TABLE 25

| En | Structure | IUPAC Name | MW | Formula |
|---|---|---|---|---|
| 1 | 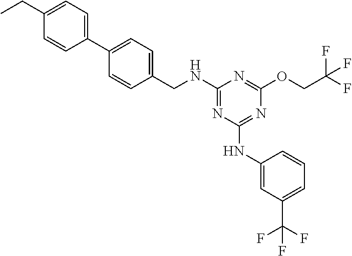 | N-[(4′-ethyl-1,1′-biphenyl-4-yl)methyl]-6- (2,2,2-trifluoroethoxy)-N′-[3- (trifluoromethyl)phenyl]-1,3,5-triazine- 2,4-diamine | 547.5 | C27H23F6N5O |
| 2 | 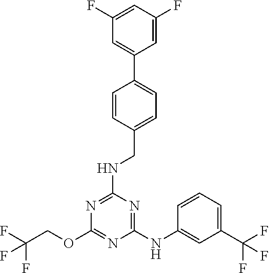 | N-[(3″,5″-difluorobiphenyl-4-yl)methyl]-6- (2,2,2-trifluoroethoxy)-N′-[3- (trifluoromethyl)phenyl]-1,3,5-triazine- 2,4-diamine | 555.4 | C25H17F8N5O |
| 3 | 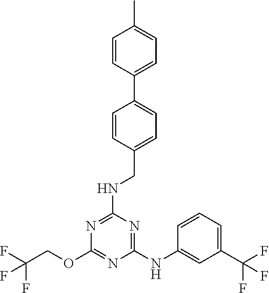 | N-[(4″-methylbiphenyl-4-yl)methyl]-6- (2,2,2-trifluoroethoxy)-N′-[3- (trifluoromethyl)phenyl]-1,3,5-triazine- 2,4-diamine | 533.5 | C26H21F6N5O |
| 4 | 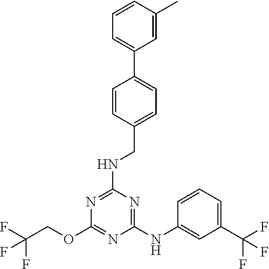 | N-[(3″-methylbiphenyl-4-yl)methyl]-6- (2,2,2-trifluoroethoxy)-N′-[3- (trifluoromethyl)phenyl]-1,3,5-triazine- 2,4-diamine | 533.5 | C26H21F6N5O |
| 5 | 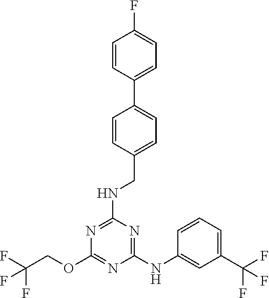 | N-[(4″-fluorobiphenyl-4-yl)methyl]-6- (2,2,2-trifluoroethoxy)-N′-[3- (trifluoromethyl)phenyl]-1,3,5-triazine- 2,4-diamine | 537.4 | C25H18F7N5O |
| 6 | 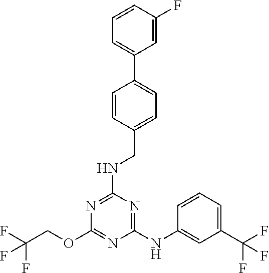 | N-[(3″-fluorobiphenyl-4-yl)methyl]-6- (2,2,2-trifluoroethoxy)-N′-[3- (trifluoromethyl)phenyl]-1,3,5-triazine- 2,4-diamine | 537.4 | C25H18F7N5O |
| 7 | 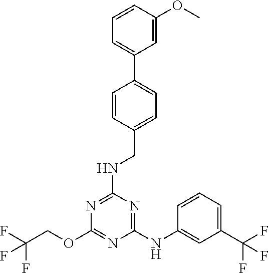 | N-[(3″-methoxybiphenyl-4-yl)methyl]-6- (2,2,2-trifluoroethoxy)-N′-[3- (trifluoromethyl)phenyl]-1,3,5-triazine- 2,4-diamine | 549.5 | C26H21F6N5O2 |
| 8 | 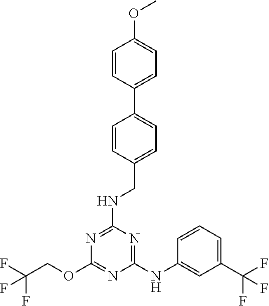 | N-[(4″-methoxybiphenyl-4-yl)methyl]-6- (2,2,2-trifluoroethoxy)-N′-[3- (trifluoromethyl)phenyl]-1,3,5-triazine- 2,4-diamine | 549.5 | C26H21F6N5O2 |
| 9 | 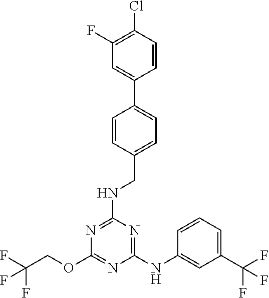 | N-[(4″-chloro-3″-fluorobiphenyl-4- yl)methyl]-6-(2,2,2-trifluoroethoxy)-N′-[3- (trifluoromethyl)-phenyl]-1,3,5-triazine- 2,4-diamine | 571.9 | C25H17ClF7N5O |
| 10 | 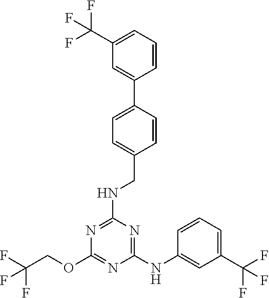 | 6-(2,2,2-trifluoroethoxy)-N-{[3″- (trifluoromethyl)-biphenyl-4-yl]methyl}- N′-[3-(trifluoromethyl)-phenyl]-1,3,5- triazine-2,4-diamine | 587.4 | C26H18F9N5O |
| 11 | 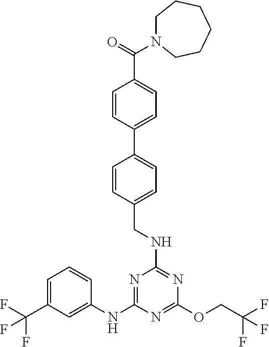 | 6-(2,2,2-trifluoroethoxy)-N-[3-(2,2,2- trifluoro-ethoxy)phenyl]-N′-[3- (trifluoromethyl)phenyl]-1,3,5-triazine- 2,4-diamine | 644.6 | C32H30F6N6O2 |
| 12 | 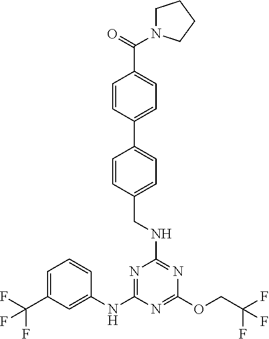 | 2-methyl-1-{4-[3-({[4-(2,2,2- trifluoroethoxy)-6-{[3- (trifluoromethyl)phenyl]amino}-1,3,5- triazin-2- yl]amino}methyl)phenyl]piperazin-1- yl}propan-1-one | 616.6 | C30H26F6N6O2 |
| 13 | 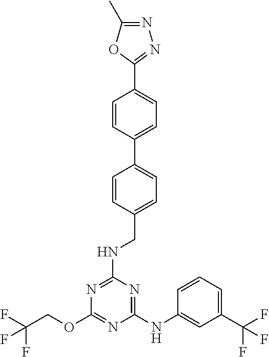 | N-{[4″-(5-methyl-1,3,4-oxadiazol-2- yl)biphenyl-4-yl]methyl}-6-(2,2,2- trifluoroethoxy)-N′-[3-(tri- fluoromethyl)phenyl]-1,3,5-triazine-2,4- diamine | 601.5 | C28H21F6N7O2 |
| 14 | 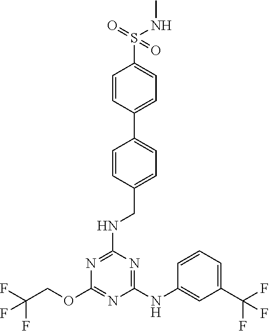 | N-methyl-4″-({[4-(2,2,2-trifluoroethoxy)- 6-{[3-(trifluoromethyl)phenyl]amino}- 1,3,5-triazin-2-yl]amino}methyl)biphenyl- 4-sulfonamide | 613.6 | C26H22F6N6O3S |
| 15 | 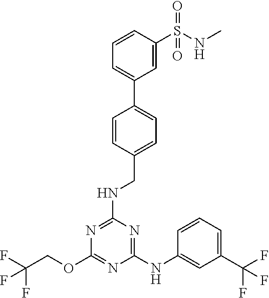 | N-methyl-4″-({[4-(2,2,2-trifluoroethoxy)- 6-{[3-(trifluoromethyl)phenyl]amino}- 1,3,5-triazin-2-yl]amino}methyl)biphenyl- 3-sulfonamide | 613.6 | C26H22F6N6O3S |
| 16 | 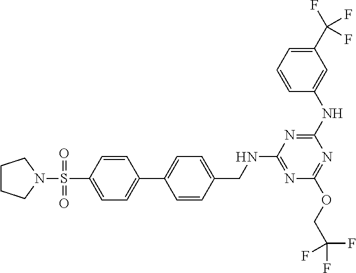 | N-{[4′-(pyrrolidin-1-ylsulfonyl)-1,1′- biphenyl-4-yl]methyl}-6-(2,2,2- trifluoroethoxy)-N′-[3-(trifluoro- methyl)phenyl]-1,3,5-triazine-2,4-diamine | 652.6 | C29H26F6N6O3S |
| 17 | 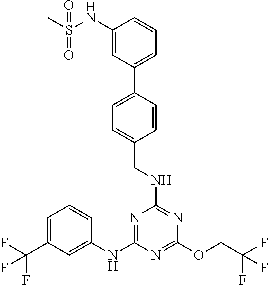 | N-[4″-({[4-(2,2,2-trifluoroethoxy)-6-{[3- (trifluoro-methyl)phenyl]amino}-1,3,5- triazin-2-yl]amino}-methyl)biphenyl-3- yl]methanesulfonamide | 613.6 | C26H22F6N6O3S |
| 18 | 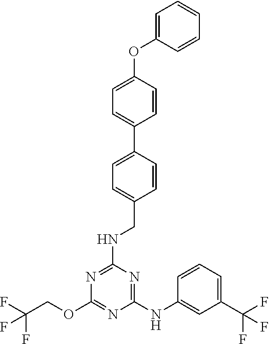 | N-[(4″-phenoxybiphenyl-4-yl)methyl]-6- (2,2,2-trifluoroethoxy)-N′-[3- (trifluoromethyl)phenyl]-1,3,5-triazine- 2,4-diamine | 611.5 | C31H23F6N5O2 |
| 19 | 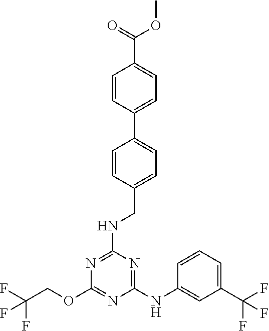 | methyl 4″-({[4-(2,2,2-trifluoroethoxy)-6- {[3-(trifluoromethyl)phenyl]amino}-1,3,5- triazin-2-yl]amino}methyl)biphenyl-4- carboxylate | 577.5 | C27H21F6N5O3 |
| 20 | 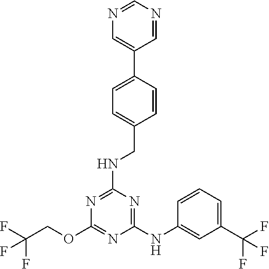 | 4-methyl-N-[4-({[4-(2,2,2- trifluoroethoxy)-6-{[3- (trifluoromethyl)phenyl]amino}-1,3,5- triazin-2- yl]amino}methyl)phenyl]benzamide | 521.4 | C23H17F6N7O |
| 21 | 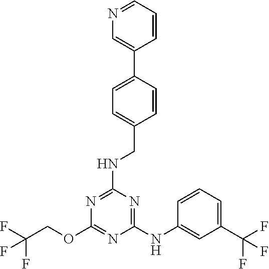 | 2-methyl-N-[4-({[4-(2,2,2- trifluoroethoxy)-6-{[3- (trifluoromethyl)phenyl]amino}-1,3,5- triazin-2- yl]amino}methyl)phenyl]benzamide | 520.4 | C24H18F6N6O |
| 22 | 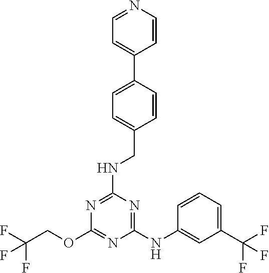 | 2-methyl-N-[4-({[4-(2,2,2- trifluoroethoxy)-6-{[3- (trifluoromethyl)phenyl]amino}-1,3,5- triazin-2- yl]amino}methyl)phenyl]propanamide | 520.4 | C24H18F6N6O |

### Procedures and Analytical Data for Table 25.

All analogs were prepared by Library 25 procedures.

### 1. N-[(4′-ethyl-1,1′-biphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 1.14 m (3H), 2.60 m (2H), 4.66 m (2H), 4.98 m (2H), 7.22 m (3H), 7.5 m (7H), 7.88 m (2H), 8.18 s (1H), 9.42 s (1H); LCMS: M+1=548

### 2. N-[(3″,5″-difluorobiphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 4.68 m (2H), 4.98 m (2H), 7.06 m (1H), 7.32 m (3H), 7.42 m (3H), 7.62 d (2H), 7.88 m (2H), 8.18 s (1H), 9.42 s (1H); LCMS: M+1=556

### 3. N-[(4″-methylbiphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 2.30 s (3H), 4.68 m (2H), 4.98 m (2H), 7.30 m (5H), 7.50 d (4H), 7.6 s (1H), 7.98 bs (2H), 8.2 bs (1H), 9.65 s (1H); LCMS: M+1=534

### 4. N-[(3″-methylbiphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 2.30 s (3H), 4.66 m (2H), 4.98 m (2H), 7.18 d (1H), 7.4 m (9H), 7.88 m (2H), 8.18 s (1H), 9.42 s (1H); LCMS: M+1=534

### 5. N-[(4″-fluorobiphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 4.70 m (2H), 4.98 m (2H), 7.2 t (2H), 7.32 d (1H), 7.58 m (7H), 7.88 m (2H), 8.18 s (1H), 9.40 s (1H); LCMS: M+1=538

### 6. N-[(3″-fluorobiphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 4.68 m (2H), 4.98 m (2H), 7.18 m (1H), 7.32 d (1H), 7.46 m (6H), 7.62 d (2H), 7.88 m (2H), 8.18 s (1H), 9.46 s (1H); LCMS: M+1=538

### 7. N-[(3″-methoxybiphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 3.8 s (3H), 4.70 m (2H), 4.98 m (2H), 6.94 d (1H), 7.2 t (2H), 7.4 m (5H), 7.6 d (2H), 7.88 m (2H), 8.18 s (1H), 9.42 s (1H); LCMS: M+1=550

### 8. N-[(4′-methoxy-1,1′-biphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 3.82 s (3H), 4.66 m (2H), 4.98 m (2H), 7.0 d (2H), 7.3 d (1H), 7.38 d (2H), 7.56 m (5H), 7.88 m (2H), 8.18 s (1H), 9.42 s (1H); LCMS: M+1=550

### 9. N-[(4″-chloro-3″-fluorobiphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 4.64 m (2H), 4.98 m (2H), 7.32 m (1H), 7.58 m (8H), 7.86 m (2H), 8.18 s (1H), 9.65 s (1H); LCMS: M+1=572

### 10. 6-(2,2,2-trifluoroethoxy)-N-{[3″-(trifluoromethyl)biphenyl-4-yl]methyl}-N′-[3-(trifluoromethyl)phenyl]-1,3,5-triazine-2,4-diamine

1H NMR (DMSO, ppm) 4.64 m (2H), 4.98 m (2H), 7.34 d (1H), 7.48 m (3H), 7.68 m (4H), 8.0 m (4H), 8.18 s (1H), 9.65 s (1H); LCMS: M+1=588

### 11. 6-(2,2,2-trifluoroethoxy)-N-[3-(2,2,2-trifluoroethoxy)phenyl]-N′-[3-(trifluoromethyl)phenyl]-1,3,5-triazine-2,4-diamine

LCMS: M+1=645.7 NMR 1H, DMSO-d6 δ, ppm: 1.65 d (8H); 3.50 s (4H); 4.61 d (2H); 4.97 m (2H); 7.30 d (1H); 7.38-7.55 m (6H); 7.60-7.70 m (4H); 7.85-8.05 bm (2H); 8.15 bm (1H); 9.60 bs (1H).

### 12. 2-methyl-1-{4-[3-{[4-(2,2,2-trifluoroethoxy)-6-{[3-(trifluoromethyl)phenyl]amino}-1,3,5-triazin-2-yl]amino}methyl)phenyl]piperazin-1-yl}propan-1-one

LCMS: M+1=617.6 NMR 1H, DMSO-d6 δ, ppm: 1.85 d (4H); 3.50 m (4H); 4.61 d (2H); 4.98 m (2H); 7.30 d (1H); 7.40-7.70 m (9H); 7.85-8.05 bm (2H); 8.15 bm (1H); 9.65 bs (1H).

### 13. N-{[4″-(5-methyl-1,3,4-oxadiazol-2-yl)biphenyl-4-yl]methyl}-6-(2,2,2-trifluoroethoxy)-N′-[3-(trifluoromethyl)phenyl]-1,3,5-triazine-2,4-diamine

1H NMR (DMSO, ppm) 2.6 s (3H), 4.70 d (2H), 4.98 m (2H), 7.34 d (1H), 7.48 m (3H), 7.7 d (2H), 7.8 d (2H), 7.96 bs (1H), 8.1 d (2H), 8.2 bs (1H), 9.55 bs (1H); LCMS: M+1=602

### 14. N-methyl-4″-{[4-(2,2,2-trifluoroethoxy)-6-{[3-(trifluoromethyl)phenyl]amino}-1,3,5-triazin-2-yl]amino}methyl)biphenyl-4-sulfonamide

1H NMR (DMSO, ppm) 3.0 s (3H), 4.68 m (2H), 4.98 m (2H), 7.18 s (1H), 7.32 d (1H), 7.46 m (3H), 7.64 d (2H), 7.8 m (6H), 7.88 s (1H), 9.45 s (1H); LCMS: M+1=613

### 15. N-methyl-4″-{[4-(2,2,2-trifluoroethoxy)-6-{[3-(trifluoromethyl)phenyl]amino}-1,3,5-triazin-2-yl]amino}methyl)biphenyl-3-sulfonamide

1H NMR (DMSO, ppm) 4.70 d (2H), 4.98 m (2H), 7.18 bs (1H), 7.3 d (1H), 7.44 m (3H), 7.68 m (3H), 7.78 d (1H), 7.98 m (4H), 8.2 bs (1H), 9.48 bs (1H); LCMS: M+1=613

### 16. N-{[4′-(pyrrolidin-1-ylsulfonyl)-1,1′-biphenyl-4-yl]methyl}-6-(2,2,2-trifluoroethoxy)-N′-[3-(trifluoromethyl)phenyl]-1,3,5-triazine-2,4-diamine

1H NMR (DMSO, ppm) 1.80 m (4H), 3.20 m (4H), 4.64 m (2H), 4.98 m (2H), 7.32 d (2H), 7.48 m (3H), 7.68 d (2H), 8.0 m (7H), 9.45 s (1H); LCMS: M+1=653

### 17. N-[(4″-phenoxybiphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 1.18 m (4H), 3.20 m (4H), 4.60 m (2H), 4.98 m (2H), 7.32 d (1H), 7.48 m (3H), 7.70 m (4H), 7.88 d (2H), 8.02 bs (1H), 8.18 s (1H), 9.65 s (1H); LCMS: M+1=653

### 18. N-(4′-{[(4-(2,2,2-trifluoroethoxy)-6-{[3-(trifluoromethyl)phenyl]amino}-1,3,5-triazin-2-yl)amino]methyl}-1,1′-biphenyl-3-yl)methanesulfonamide

1H NMR (DMSO, ppm) 3.0 s (3H), 4.66 d (2H), 4.98 m (2H), 7.22 d (1H), 7.0 (1H), 7.4 m (4H), 7.3 d (1H), 7.98 bs (2H), 8.18 bs (1H), 9.3 s (1H), 9.4 s (1H); LCMS: M+1=613

### 19. N-[(4″-phenoxybiphenyl-4-yl)methyl]-6-(2,2,2-trifluoroethoxy)-N′-[3-(trifluoromethyl)phenyl]-1,3,5-triazine-2,4-diamine

1H NMR (DMSO, ppm) 4.66 m (2H), 4.98 m (2H), 7.02 m (4H), 7.88 t (3H), 7.42 m (7H), 7.6 d (3H), 7.98 bs (2H), 8.18 s (1H), 9.65 s (1H); LCMS: M+1=612

### 20. methyl 4″-{[4-(2,2,2-trifluoroethoxy)-6-{[3-(trifluoromethyl)phenyl]amino}-1,3,5-triazin-2-yl]amino}methyl)biphenyl-4-carboxylate

1H NMR (DMSO, ppm) 3.90 s (3H), 4.72 m (2H), 4.98 m (2H), 7.36 d (1H), 7.48 m (3H), 7.68 d (2H), 7.78 d (2H), 8.0 m (5H), 9.46 s (1H); LCMS: M+1=678

### 21. 4-methyl-N-[4-{[4-(2,2,2-trifluoroethoxy)-6-{[3-(trifluoromethyl)phenyl]amino}-1,3,5-triazin-2-yl]amino}methyl)phenyl]benzamide

LCMS: M+1=522.6 NMR 1H, DMSO-d6 δ, ppm: 4.64 d (2H); 4.97 m (2H); 7.29 d (1H); 7.50 m (3H); 7.71 m (2H) 7.85-8.05 bm (2H); 8.15 bm (1H); 9.05 s (2H); 9.12 s (1H); 9.60 bs (1H).

### 22. N-(4-pyridin-3-ylbenzyl)-6-(2,2,2-trifluoroethoxy)-N′-[3-(trifluoromethyl)phenyl]-1,3,5-triazine-2,4-diamine

LCMS: M+1=521.6 NMR 1H, DMSO-d6 δ, ppm: 4.60 d (2H); 4.95 m (2H); 7.30 d (1H); 7.42-7.69 m (8H); 7.85-8.15 bm (4H); 8.54 d (1H); 8.87 s (1H); 9.63 bs (1H).

### 23. 2-methyl-N-[4-{[4-(2,2,2-trifluoroethoxy)-6-{[3-(trifluoromethyl)phenyl]amino}-1,3,5-triazin-2-yl]amino}methyl)phenyl]propanamide

LCMS: M+1=521.6 NMR 1H, DMSO-d6 δ, ppm: 4.62 d (2H); 4.93 m (2H); 7.28 d (1H); 7.47-7.53 m (3H); 7.69-7.80 m (4H); 7.87-8.15 bm (4H); 8.65 d (1H); 9.62 bs (1H).

### Generic Synthesis of Intermediates and Final Compounds for Table 26 (see Table 29).

TABLE 26

| En | Structure | IUPAC Name | MW | Formula |
|---|---|---|---|---|
| 1 | 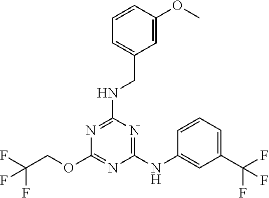 | N-(3-methoxybenzyl)-6-(2,2,2- trifluoroethoxy)-N′-[3- (trifluoromethyl)phenyl]-1,3,5-triazine- 2,4-diamine | 473.4 | C₂₀H₁₇F₆N₅O₂ |
| 2 | 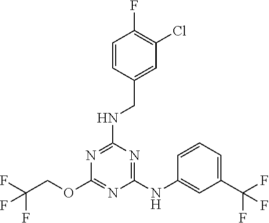 | N-(3-chloro-4-fluorobenzyl)-6-(2,2,2- trifluoroethoxy)-N′-[3- (trifluoromethyl)phenyl]-1,3,5-triazine- 2,4-diamine | 495.8 | C₁₉H₁₃ClF₇N₅O |
| 3 | 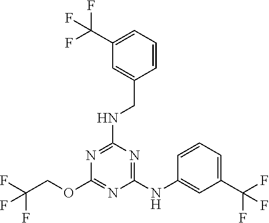 | 6-(2,2,2-trifluoroethoxy)-N-[3- (trifluoromethyl)benzyl]-N′-[3- (trifluoromethyl)phenyl]-1,3,5-triazine- 2,4-diamine | 511.3 | C₂₀H₁₄F₉N₅O |
| 4 | 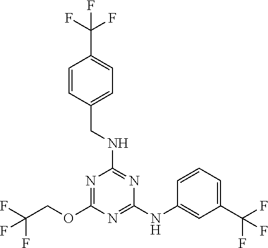 | 6-(2,2,2-trifluoroethoxy)-N-[4- (trifluoromethyl)benzyl]-N′-[3- (trifluoromethyl)phenyl]-1,3,5-triazine- 2,4-diamine | 511.3 | C₂₀H₁₄F₉N₅O |
| 5 | 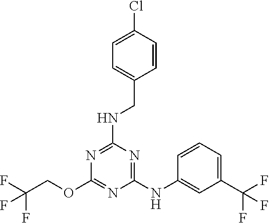 | N-(4-chlorobenzyl)-6-(2,2,2- trifluoroethoxy)-N′-[3- (trifluoromethyl)phenyl]-1,3,5-triazine- 2,4-diamine | 477.8 | C₁₉H₁₄ClF₆N₅O |
| 6 | 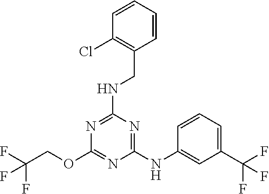 | N-(2-chlorobenzyl)-6-(2,2,2- trifluoroethoxy)-N′-[3- (trifluoromethyl)phenyl]-1,3,5-triazine- 2,4-diamine | 477.8 | C₁₉H₁₄ClF₆N₅O |
| 7 | 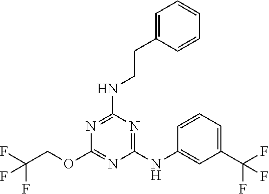 | N-(2-phenylethyl)-6-(2,2,2- trifluoroethoxy)-N′-[3- (trifluoromethyl)phenyl]-1,3,5-triazine- 2,4-diamine | 457.4 | C₂₀H₁₇F₆N₅O |
| 8 | 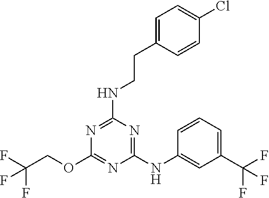 | N-[2-(4-chlorophenyl)ethyl]-6-(2,2,2- trifluoroethoxy)-N′-[3- (trifluoromethyl)phenyl]-1,3,5-triazine- 2,4-diamine | 491.8 | C₂₀H₁₆ClF₆N₅O |

### Procedures and Analytical Data for Table 26.

Entries 1 to 8 are from Library 29a (See Table 29 for procedures).

### 1. N-(3-methoxybenzyl)-6-(2,2,2-trifluoroethoxy)-N′-[3-(trifluoromethyl)phenyl]-1,3,5-triazine-2,4-diamine

LCMS: M+1=473.3; 1H NMR (DMSO-d6, 90° C., ppm): d=3.74 s (3H), 4.56 d (2H), 4.92 q (2H), 6.90 m (3H), 7.28 m (2H), 7.48 t (1H), 7.92 m (2H), 8.14 s (1H), 9.52 s (1H).

### 2. N-(3-chloro-4-fluorobenzyl)-6-(2,2,2-trifluoroethoxy)-N′-[3-(trifluoromethyl)phenyl]-1,3,5-triazine-2,4-diamine

LCMS: M+1=495.7; 1H NMR (DMSO-d6, 90° C., ppm): d=4.52 d (2H), 4.92 q (2H), 7.40 m (5H), 8.02 m (3H), 9.70 s (1H).

### 3. 6-(2,2,2-trifluoroethoxy)-N-[3-(trifluoromethyl)benzyl]-N′-[3-(trifluoromethyl)phenyl]-1,3,5-triazine-2,4-diamine

LCMS: M+1=511.3; 1H NMR (DMSO-d6, 90° C., ppm): d=4.68 d (2H), 4.92 q (2H), 7.28 d (2H), 7.56 m (5H), 8.10 m (3H), 9.68 s (1H).

### 4. 6-(2,2,2-trifluoroethoxy)-N-[4-(trifluoromethyl)benzyl]-N′-[3-(trifluoromethyl)phenyl]-1,3,5-triazine-2,4-diamine

LCMS: M+1=511.3; 1H NMR (DMSO-d6, 90° C., ppm): d=4.6 (2H, d); 4.8 (2H, q); 7.25 (1H, d); 7.4 (1H, t); 7.6 (4H, q); 7.85 (2H, s); 8.1 (1H, s); 9.5 (1H, s).

### 5. N-(4-chlorobenzyl)-6-(2,2,2-trifluoroethoxy)-N′-[3-(trifluoromethyl)phenyl]-1,3,5-triazine-2,4-diamine

LCMS: M+1=477.7; 1H NMR (DMSO-d6, 90° C., ppm): d=4.55 (2H, d); 4.9 (2H, q); 7.35 (5H, m); 7.5 (1H, t); 8.0 (3H, m); 9.6 (1H, s).

### 6. N-(2-chlorobenzyl)-6-(2,2,2-trifluoroethoxy)-N′-[3-(trifluoromethyl)phenyl]-1,3,5-triazine-2,4-diamine

LCMS: M+1=477.7; 1H NMR (DMSO-d6, 90° C., ppm): d=4.65 (2H, d); 4.9 (2H, q); 7.4 (6H, m); 7.9 (3H, m); 9.5 (1H, s).

### 7. N-(2-phenylethyl)-6-(2,2,2-trifluoroethoxy)-N′-[3-(trifluoromethyl)phenyl]-1,3,5-triazine-2,4-diamine

LCMS: M+1=457.3; 1H NMR (DMSO-d6, 90° C., ppm): 2.97 (m, 2H), 3.62 (m, 2H), 4.99 (m, 2H), 7.38 (m, 8H), 8.04 (m, 2H), 9.62 (s, 1H).

### 8. N-[2-(4-chlorophenyl)ethyl]-6-(2,2,2-trifluoroethoxy)-N′-[3-(trifluoromethyl)phenyl]-1,3,5-triazine-2,4-diamine

LCMS: M+1=491.8; 1H NMR (DMSO-d6, 90° C., ppm): 2.88 (m, 2H), 3.65 (m, 2H), 4.92 (m, 2H), 7.42 (m, 8H), 7.98 (m, 1H), 8.45 (s, 1H).

### Generic Synthesis of Intermediates and Final Compounds for Table 27

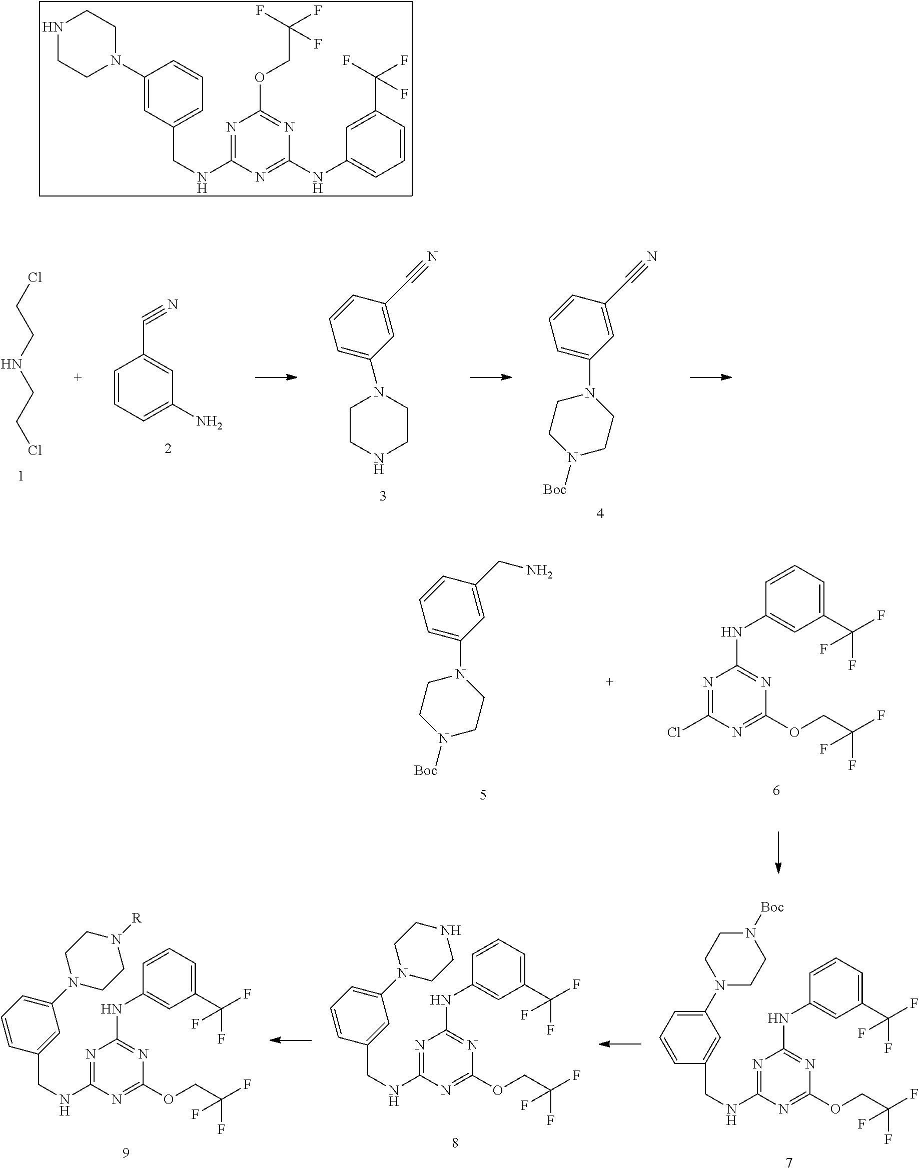

Preparation of 3. To 200 ml of water are added 20.0 g of 2 and 3 g of sodium carbonate and the mixture was dissolved by heating. To the solution was added 40 g of bis(2-chloroethyl)amine hydrochloride 1 and the mixture was refluxed by heating for 3 hours, And then, a suspension containing 13 g of sodium carbonate suspended in 30 ml of water was added thereto, and the mixture was refluxed by heating for 12 hours and then stirred for 6 hours at room temperature. Precipitate was filtered off and then dissolved in 100 mL of water and adjusted to ph 10 by addition of alkali. The precipitate was filtered off, dissolved in toluene and all solvents were removed under reduced pressure. Product was used for the next step without additional purification.

Preparation of 4. 3 (4 g, 21 mmol) was dissolved in DMF (50 ml) and 3 BOC-anhydride (45 mmol) was added and reaction mixture was stirred at 60° C. overnight. Solvents were removed under reduced pressure and the resulting precipitate was washed with ether.

Preparation of 5. Intermediate 4 (5 g) was dissolved in THF (100 ml) and hydrogenated in presence of Raney nickel (1 g) and 25% aqueous ammonia (5 ml) under 2 atm at room temperature overnight. Then catalyst was filtered off and solvent was removed under reduced pressure. The resulting oil was used for the next step without additional purification.

Preparation of 7. Intermediate 5 (4.1 g, 14 mmol) was mixed with 6 (14 mmol, 5.2 g) in 1,4-dioxane (100 ml) and triethylamine (15 mmol) was added. Reaction mixture was stirred at 60° C. overnight. Then reaction mixture was diluted cold with water (500 ml) and precipitate was filtered off.

Preparation of 8. Intermediate 7 (5.5) was dissolved in 1,4-dioxane*HCl (50 ml) and stirred at room temperature overnight. Then precipitate was filtered, washed with ether, and collected.

Preparation of 9: With acids: Method A. An appropriate acid (0.05 mmol) was dissolved in 1,4-dioxane (3 ml) and stirred with CDI (0.055 mmol) at 80° C. for 1.5 hours. Then amine 8 (0.05 mmol) was added and reaction mixture was stirred at this temperature overnight. Then solvent was removed under reduced pressure and residue was washed with water and purified by HPLC. Method B. Acid hydrochloride (0.05 mmol) was dissolved in 1,4-dioxane (3 ml). amine 8 (0.05 mmol) and EDC (0.06 mmol) and triethylamine (0.15 mmol) were added and reaction mixture was stirred at room temperature overnight. Then the solvent was removed under reduced pressure, and the residue was washed with water and purified by HPLC.

With alkylators: Amine 8 (0.05 mmol) was mixed with an appropriate alkylating agent (0.06 mmol) in 3 ml of CH₃CN. Potassium carbonate (0.1 mmol) was added and reaction mixture was stirred at room temperature overnight. Then reaction mixture was diluted with water and extracted with 20 mL of chloroform. Organic layer was separated and solvent was removed under reduced pressure. The residue was purified by HPLC.

With sufl° Chlorides: Amine 8 (0.05 mmol) was mixed with appropriate sulf° Chloride (0.06 mmol) in 1,4-dioxane (3 ml) and triethylamine (0.12 mmol) was added. The reaction mixture was stirred at room temperature overnight. Then solvent was removed under reduced pressure and the residue was washed with water and purified by HPLC.

TABLE 27

| En | Structure | IUPAC Name | MW | Formula |
|---|---|---|---|---|
| 1 | 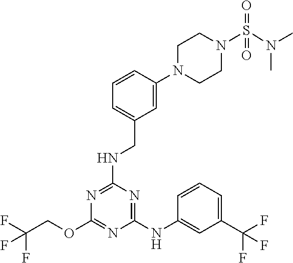 | N,N-dimethyl-4-[3-({[4-(2,2,2- trifluoroethoxy)-6-{[3- (trifluoromethyl)phenyl]amino}-1,3,5- triazin-2- yl]amino}methyl)phenyl]piperazine-1- sulfonamide | 635.6 | C25H28F6N8O3S |
| 2 | 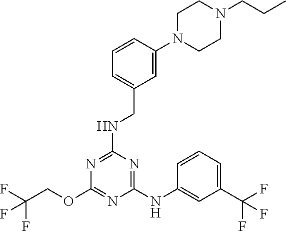 | N-[3-(4-propylpiperazin-1-yl)benzyl]-6- (2,2,2-trifluoroethoxy)-N′-[3- (trifluoromethyl)phenyl]-1,3,5-triazine- 2,4-diamine | 569.5 | C26H29F6N7O |
| 3 | 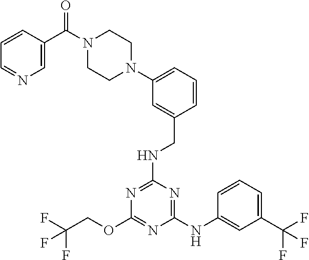 | pyridin-3-yl{4-[3-({[4-(2,2,2- trifluoroethoxy)-6-{[3- (trifluoromethyl)phenyl]amino}-1,3,5- triazin-2- yl]amino}methyl)phenyl]piperazin-1- yl}methanone | 632.6 | C29H26F6N8O2 |
| 4 | 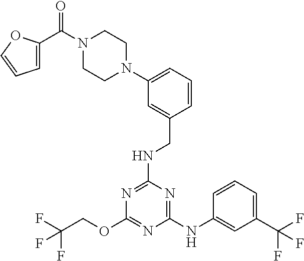 | furan-2-yl{4-[3-({[4-(2,2,2- trifluoroethoxy)-6-{[3- (trifluoromethyl)phenyl]amino}-1,3,5- triazin-2- yl]amino}methyl)phenyl]piperazin-1- yl}methanone | 621.5 | C28H25F6N7O3 |
| 5 | 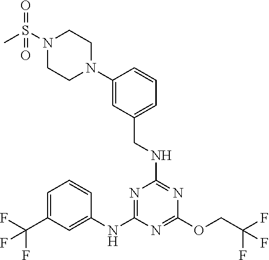 | N-{3-[4-(methylsulfonyl)piperazin-1- yl]benzyl}-6-(2,2,2-trifluoroethoxy)-N′- [3-(trifluoromethyl)phenyl]-1,3,5- triazine-2,4-diamine | 606.6 | C24H25F6N7O3S |
| 6 | 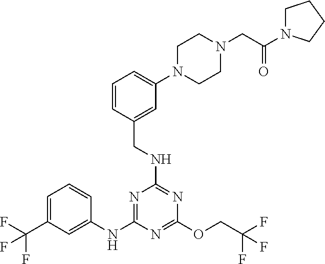 | 1-(pyrrolidin-1-yl)-2-{4-[3-({[4-(2,2,2- trifluoro-ethoxy)-6-{[3- (trifluoromethyl)phenyl]amino}-1,3,5- triazin-2- yl]amino}methyl)phenyl]piperazin-1- yl}ethanone | 638.6 | C29H32F6N8O2 |
| 7 | 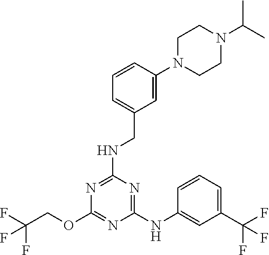 | N-{3-[4-(propan-2-yl)piperazin-1- yl]benzyl}-6-(2,2,2-trifluoroethoxy)-N′- [3-(trifluoromethyl)phenyl]-1,3,5- triazine-2,4-diamine | 569.5 | C26H29F6N7O |
| 8 | 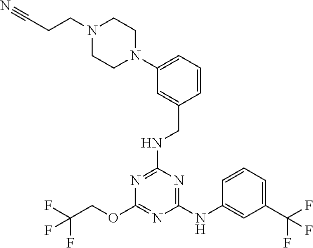 | 3-{4-[3-({[4-(2,2,2-trifluoroethoxy)-6-{[3- (trifluoro-methyl)phenyl]amino}-1,3,5- triazin-2-yl]amino}- methyl)phenyl]piperazin-1- yl}propanenitrile | 580.5 | C26H26F6N8O |
| 9 | 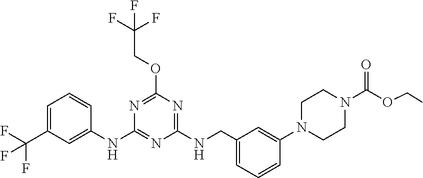 | ethyl 4-[3-({[4-(2,2,2-trifluoroethoxy)-6- {[3-(trifluoromethyl)phenyl]amino}-1,3,5- triazin-2- yl]amino}methyl)phenyl]piperazine-1- carboxylate | 599.5 | C26H27F6N7O3 |
| 10 | 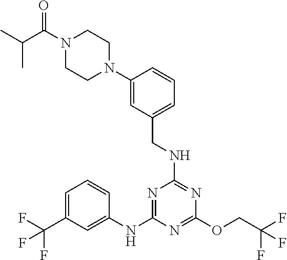 | N-[3-(pyridin-4-yl)benzyl]-6-(2,2,2- trifluoroethoxy)-N′-[3- (trifluoromethyl)phenyl]-1,3,5-triazine- 2,4-diamine | 597.6 | C27H29F6N7O2 |
| 11 | 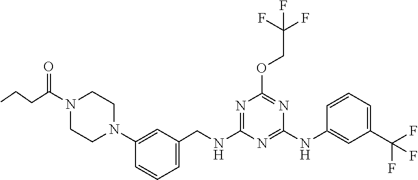 | N-[4-(pyrimidin-5-yl)benzyl]-6-(2,2,2- trifluoroethoxy)-N′-[3- (trifluoromethyl)phenyl]-1,3,5-triazine- 2,4-diamine | 597.6 | C27H29F6N7O2 |
| 12 | 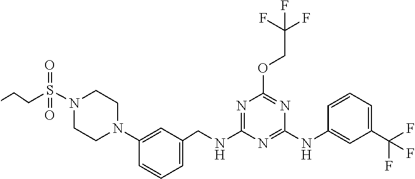 | N-{4-[4-(pyridin-2-ylmethyl)piperazin-1- yl]benzyl}-6-(2,2,2-trifluoroethoxy)-N′- [3-(trifluoromethyl)phenyl]-1,3,5- triazine-2,4-diamine | 634.6 | C26H29F6N7O3S |
| 13 | 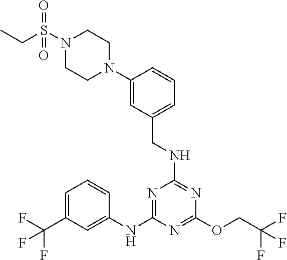 | N-(propan-2-yl)-2-{4-[4-({[4-(2,2,2- trifluoro-ethoxy)-6-{[3- (trifluoromethyl)phenyl]amino}-1,3,5- triazin-2- yl]amino}methyl)phenyl]piperazin-1- yl}acetamide | 620.6 | C25H27F6N7O3S |
| 14 | 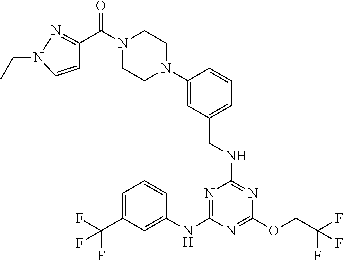 | N-[3-(morpholin-4-ylmethyl)benzyl]-6- (2,2,2-trifluoroethoxy)-N′-[3 (trifluoromethyl)phenyl]-1,3,5-triazine- 2,4-diamine | 649.6 | C29H29F6N9O2 |
| 15 | 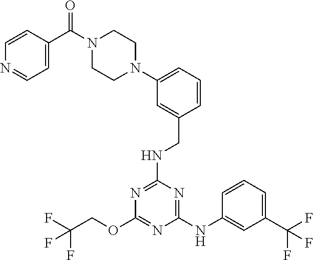 | N-(1H-indol-5-ylmethyl)-6-(2,2,2- trifluoroethoxy)-N′-[3- (trifluoromethyl)phenyl]-1,3,5-triazine- 2,4-diamine | 632.6 | C29H26F6N8O2 |
| 16 | 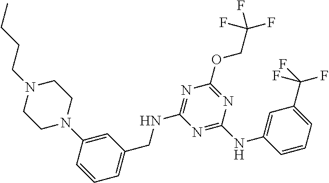 | N-[3-(4-butylpiperazin-1-yl)benzyl]-6- (2,2,2-trifluoroethoxy)-N′-[3- (trifluoromethyl)phenyl]-1,3,5-triazine- 2,4-diamine | 583.6 | C27H31F6N7O |
| 17 | 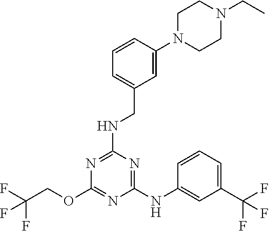 | N2-(3-(4-ethylpiperazin-1-yl)benzyl)-6- (2,2,2-trifluoroethoxy)-N4-(3- (trifluoromethyl)phenyl)-1,3,5-triazine- 2,4-diamine | 555.5 | C25H27F6N7O |
| 18 | 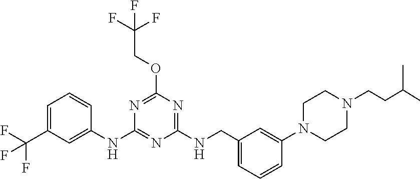 | N2-(3-(4-isopentylpiperazin-1- yl)benzyl)-6-(2,2,2-trifluoroethoxy)-N4- (3-(trifluoromethyl)phenyl)-1,3,5- triazine-2,4-diamine | 597.6 | C28H33F6N7O |
| 19 | 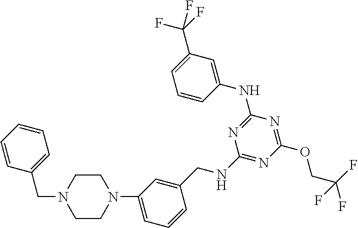 | N-[3-(4-benzylpiperazin-1-yl)benzyl]-6- (2,2,2-trifluoroethoxy)-N′-[3- (trifluoromethyl)phenyl]-1,3,5-triazine- 2,4-diamine | 617.6 | C30H29F6N7O |

### Procedures and Analytical Data for Table 27.

All compounds were prepared by the above procedures.

### 1. N,N-dimethyl-4-[3-{[4-(2,2,2-trifluoroethoxy)-6-{[3-(trifluoromethyl)phenyl]amino}-1,3,5-triazin-2-yl]amino}methyl)phenyl]piperazine-1-sulfonamide

1H NMR (DMSO, ppm) 2.88 s (6H), 3.1 d (4H), 3.18 d (4H), 4.56 d (2H), 4.98 m (2H), 6.8 m (2H), 7.0 s (1H), 7.18 t (1H), 7.32 d (1H), 7.58 t (1H), 7.88 bs (2H), 8.2 bs (1H), 9.82 bs (1H); LCMS: M+1=635

### 2. N-[3-(4-propylpiperazin-1-yl)benzyl]-6-(2,2,2-trifluoroethoxy)-N′-[3-(trifluoromethyl)phenyl]-1,3,5-triazine-2,4-diamine

1H NMR (DMSO, ppm) 0.98 t (3H), 1.70 m (2H), 3.08 m (2H), 3.4 m (8H), 4.68 d (2H), 4.98 m (2H), 6.82 d (2H), 7.0 s (1H), 7.2 t (1H), 7.26 d (1H), 7.58 t (1H), 7.88 bs (2H), 8.12 bs (1H), 9.6 bs (1H); LCMS: M+1=570

### 3. pyridin-3-yl{4-[3-{[4-(2,2,2-trifluoroethoxy)-6-{[3-(trifluoromethyl)phenyl]amino}-1,3,5-triazin-2-yl]amino}methyl)phenyl]piperazin-1-yl}methanone

1H NMR (DMSO, ppm) 3.02 d (4H), 3.5 d (4H), 4.50 s (2H), 4.98 m (2H), 6.82 m (2H), 6.98 s (1H), 7.28 t (1H), 7.40 d (1H), 7.48 d (3H), 7.8 m (3H), 8.25 s (1H), 8.65 s (2H), 9.6 bs (1H); LCMS: M+1=633

### 4. furan-2-yl{4-[3-{[4-(2,2,2-trifluoroethoxy)-6-{[3-(trifluoromethyl)phenyl]amino}-1,3,5-triazin-2-yl]amino}methyl)phenyl]piperazin-1-yl}methanone

1H NMR (DMSO, ppm) 3.14 m (4H), 3.82 m (4H), 4.32 d (2H), 4.98 m (2H), 6.70 s (1H), 6.96 t (2H), 7.0 s (2H), 7.20 t (1H), 7.40 d (1H), 7.5 t (1H), 7.70 s (1H), 7.88 bs (2H), 8.28 s (1H), 9.75 s (1H); LCMS: M+1=622

### 5. N-{3-[4-(methylsulfonyl)piperazin-1-yl]benzyl}-6-(2,2,2-trifluoroethoxy)-N′-[3-(trifluoromethyl)phenyl]-1,3,5-triazine-2,4-diamine

1H NMR (DMSO, ppm) 2.86 s (3H), 3.0 m (4H), 3.20 m (4H), 4.58 d (2H), 4.98 m (2H), 6.8 m (2H), 7.0 s (1H), 7.2 m (1H), 7.38 d (1H), 7.5 t (1H), 8.9 bs (2H), 8.2 bs (1H), 9.58 s (1H); LCMS: M+1=606

### 6. 1-(pyrrolidin-1-yl)-2-{-4-[3-{[4-(2,2,2-trifluoroethoxy)-6-{[3-(trifluoromethyl)phenyl]amino}-1,3,5-triazin-2-yl]amino}methyl)phenyl]piperazin-1-yl}ethanone

1H NMR (DMSO, ppm) 1.90 m (4H), 3.48 m (10H), 4.0 s (2H), 4.5 d (2H), 4.98 m (2H), 6.88 d (2H), 7.0 s (1H), 7.2 t (1H), 7.3 d (1H), 7.52 t (1H), 7.88 bs (2H), 8.18 bs (1H), 9.6 bs (1H); LCMS: M+1=639

### 7. N-{3-[4-(propan-2-yl)piperazin-1-yl]benzyl}-6-(2,2,2-trifluoroethoxy)-N′-[3-(trifluoromethyl)phenyl]-1,3,5-triazine-2,4-diamine

1H NMR (DMSO, ppm) 1.12 d (6H), 3.30 m (87H), 3.36 m (1H), 4.60 d (2H), 4.98 m (2H), 6.82 d (2H), 7.02 s (1H), 7.2 t (1H), 7.34 d (1H), 7.5 t (1H), 7.88 bs (2H), 8.2 bs (1H), 9.75 bs (1H); LCMS: M+1=570

### 8. 3-{4-[3-({[4-(2,2,2-trifluoroethoxy)-6-{[3-(trifluoromethyl)phenyl]amino}-1,3,5-triazin-2-yl]amino}methyl)phenyl]piperazin-1-yl}propanenitrile

LCMS: M+1=580.5; 1H NMR (DMSO-d6, 90° C., ppm): 1=2.90 m (4H), 3.33 m (4H), 3.82 m (4H), 4.25 s (6H), 4.98 m (2H), 6.89 m (3H), 7.34 m (3H), 8.01 m (3H), 9.61 s (1H).

### 9. ethyl 4-[3-{[4-(2,2,2-trifluoroethoxy)-6-{[3-(trifluoromethyl)phenyl]amino}-1,3,5-triazin-2-yl]amino}methyl)phenyl]piperazine-1-carboxylate

1H NMR (DMSO, ppm) 1.06 m (3H), 3.05 d (4H), 3.5 d (4H), 4.05 m (2H), 4.60 s (2H), 4.98 m (2H), 6.8 m (2H), 7.0 s (1H), 7.2 t (1H), 7.3 d (1H), 7.5 t (1H), 7.88 bs (2H), 8.2 s (1H), 9.70 s (1H); LCMS: M+1=600

### 10. N-[3-(pyridin-4-yl)benzyl]-6-(2,2,2-trifluoroethoxy)-N′-[3-(trifluoromethyl)phenyl]-1,3,5-triazine-2,4-diamine

LCMS: M+1=598.5 NMR 1H, DMSO-d6 δ, ppm: 1.01 s (3H); 1.02 s (3H); 2.89 m (1H); 3.12 m (4H); 3.55 m (4H); 4.49 d (2H); 4.94 m (2H); 6.81 t (2H); 6.95 s (1H); 7.19 t (1H); 7.31 d (1H); 7.44 t (1H); 7.92 bm (2H); 8.17 bs (1H); 9.58 bs (1H).

### 11. N-[4-(pyrimidin-5-yl)benzyl]-6-(2,2,2-trifluoroethoxy)-N′-[3-(trifluoromethyl)phenyl]-1,3,5-triazine-2,4-diamine

LCMS: M+1=598.5 NMR 1H, DMSO-d6 δ, ppm: 0.85 t (3H); 1.56 m (2H); 2.30 t (2H); 3.12 m (4H); 3.55 m (4H); 4.49 d (2H); 4.94 m (2H); 6.55 t (2H); 6.86 s (1H); 7.15 t (1H); 7.30 d (1H); 7.49 t (1H); 7.74-8.00 bm (2H); 8.25-8.40 bs (1H); 9.58 bs (1H).

### 12. N-{4-[4-(pyridin-2-ylmethyl)piperazin-1-yl]benzyl}-6-(2,2,2-trifluoroethoxy)-N′-[3-(trifluoromethyl)phenyl]-1,3,5-triazine-2,4-diamine

LCMS: M+1=634.5 NMR 1H, DMSO-d6 δ, ppm: 1.04 t (3H); 1.77 m (2H); 2.94 t (2H); 3.27 d (8H); 4.52 d (2H); 4.86 q (2H); 6.81 s (2H); 6.96 s (1H); 7.16 t (1H); 7.26 d (1H); 7.44 t (1H); 7.70 s (1H); 7.93 s (1H); 8.16 s (1H); 9.51 s (1H).

### 13. N-(propan-2-yl)-2-{4-[4-({[4-(2,2,2-trifluoroethoxy)-6-{[3-(trifluoromethyl)phenyl]amino}-1,3,5-triazin-2-yl]amino}methyl)phenyl]piperazin-1-yl}acetamide

LCMS: M+1=620.6 NMR 1H, DMSO-d6 δ, ppm: 1.28 t (3H); 3.04 q (2H); 3.30 m (8H); 4.52 d (2H); 4.87 q (2H); 6.81 m (2H); 6.95 s (1H); 7.16 t (1H); 7.26 d (1H); 7.44 t (1H); 7.69 t (1H); 7.92 s (1H); 8.15 s (1H); 9.50 s (1H).

### 14. N-[3-(morpholin-4-ylmethyl)benzyl]-6-(2,2,2-trifluoroethoxy)-N′-[3-(trifluoromethyl)phenyl]-1,3,5-triazine-2,4-diamine

1H NMR (DMSO, ppm) 1.40 m (3H), 3.05 m (4H), 3.98 m (4H), 4.06 m (2H), 4.50 m (2H), 4.98 m (2H), 6.54 s (1H), 6.80 t (2H), 7.0 s (1H), 7.18 t (1H), 7.26 d (1H), 7.5 t (1H), 7.70 s (1H), 7.98 bs (2H), 8.10 bs (1H), 9.70 bs (1H); LCMS: M+1=650

### 15. N-(1H-indol-5-ylmethyl)-6-(2,2,2-trifluoroethoxy)-N′-[3-(trifluoromethyl)phenyl]-1,3,5-triazine-2,4-diamine

LCMS: M+1=633.5 NMR 1H, DMSO-d6 δ, ppm: 3.18 s (4H); 3.61 s (4H); 4.52 d (2H); 4.90 q (2H); 6.81 d (2H); 6.95 s (1H); 7.17 t (1H); 7.27 d (1H); 7.36 m (2H); 7.45 t (1H); 7.78 s (1H); 7.93 s (1H); 8.15 s (1H); 8.15 s (1H); 8.66 d (2H); 9.56 s (1H).

### 16. N-[3-(4-butylpiperazin-1-yl)benzyl]-6-(2,2,2-trifluoroethoxy)-N′-[3-(trifluoromethyl)phenyl]-1,3,5-triazine-2,4-diamine

LCMS: M+1=584.6 NMR 1H, DMSO-d6 δ, ppm: 0.92 t (3H); 1.40 m (4H); 2.34 t (2H); 3.12 s (4H); 4.51 d (2H); 4.92 q (2H); 6.74 t (2H); 6.90 s (1H); 7.13 t (1H); 7.23 d (1H); 7.49 t (1H); 7.90 m (2H); 8.16 s (1H); 9.58 s (1H).

### 17. N-2-(3-(4-ethylpiperazin-1-yl)benzyl)-6-(2,2,2-trifluoroethoxy)-N-4-(3-(trifluoromethyl)phenyl)-1,3,5-triazine-2,4-diamine

LCMS: M+1=556.4 NMR 1H, DMSO-d6 δ, ppm: 1.13 t (3H); 2.41 q (2H); 2.50 s (4H); 3.14 m (4H); 4.53 d (2H); 4.88 q (2H); 6.75 d (2H); 6.91 s (1H); 7.16 t (1H); 7.23 d (1H); 7.42 t (1H); 7.64 s (1H); 7.87 s (1H); 8.18 s (1H); 9.50 s (1H).

### 18. N-2-(3-(4-isopentylpiperazin-1-yl)benzyl)-6-(2,2,2-trifluoroethoxy)-N-4-(3-(trifluoromethyl)phenyl)-1,3,5-triazine-2,4-diamine

LCMS: M+1=598.6 NMR 1H, DMSO-d6 δ, ppm: 0.92 d (6H); 1.39 m (2H); 1.65 m (1H); 2.37 t (2H); 2.50 s (4H); 3.12 s (4H); 4.50 d (2H); 4.86 q (2H); 6.75 d (2H); 6.89 s (1H); 7.13 t (1H); 7.25 d (1H); 7.44 t (1H); 7.64 zt (1H); 7.94 s (1H); 8.15 s (1H); 9.48 s (1H).

### 19. N-[3-(4-benzylpiperazin-1-yl)benzyl]-6-(2,2,2-trifluoroethoxy)-N′-[3-(trifluoromethyl)phenyl]-1,3,5-triazine-2,4-diamine

LCMS: M+1=618.6 NMR 1H, DMSO-d6 δ, ppm: 2.50 m (4H); 3.13 t (4H); 3.54 s (2H); 4.50 d (2H); 4.93 q (2H); 6.76 t (2H); 6.91 s (1H); 7.13 t (1H); 7.26 m (1H); 7.33 d (4H); 7.48 t (1H); 7.90 m (2H); 8.16 s (1H); 9.59 S (1H).

### Generic Synthesis of Intermediates and Final Compounds for Table 28

See Library 25, Table 25.

TABLE 28

| En | Structure | IUPAC Name | MW | Formula |
|---|---|---|---|---|
| 1 | 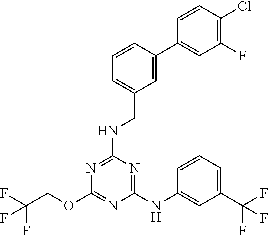 | N-[(4″-chloro-3″-fluorobiphenyl-3- yl)methyl]-6-(2,2,2- trifluoroethoxy)-N′-[3-(trifluoro- methyl)phenyl]-1,3,5-triazine-2,4- diamine | 571.9 | C25H17ClF7N5O |
| 2 | 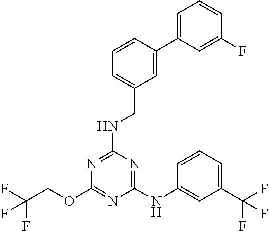 | N-[(3″-fluorobiphenyl-3-yl)methyl]- 6-(2,2,2-trifluoroethoxy)-N′-[3- (trifluoromethyl)phenyl]-1,3,5- triazine-2,4-diamine | 537.4 | C25H18F7N5O |
| 3 | 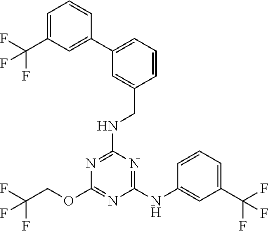 | 6-(2,2,2-trifluoroethoxy)-N-{[3″- (trifluoro-methyl)biphenyl-3- yl]methyl}-N′-[3-(trifluoro- methyl)phenyl]-1,3,5-triazine-2,4- diamine | 587.4 | C26H18F9N5O |
| 4 | 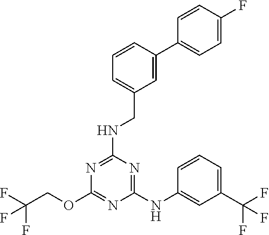 | N-[(4″-fluorobiphenyl-3-yl)methyl]- 6-(2,2,2-trifluoroethoxy)-N′-[3- (trifluoromethyl)phenyl]-1,3,5- triazine-2,4-diamine | 537.4 | C25H18F7N5O |
| 5 | 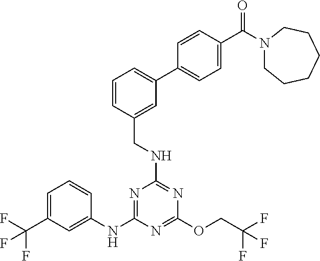 | N-(4-fluorophenyl)-4-{[4-(2,2,2- trifluoroethoxy)-6-{[3- (trifluoromethyl)phenyl]amino}- 1,3,5-triazin-2- yl]amino}benzenesulfonamide | 644.6 | C32H30F6N6O2 |
| 6 | 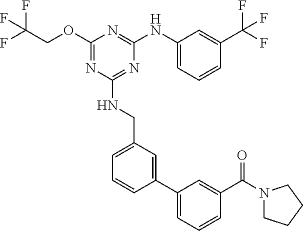 | N-{[4′-(pyrrolidin-1-ylcarbonyl)- 1,1′-biphenyl-3-yl]methyl}-6- (2,2,2-trifluoroethoxy)-N′-[3-(tri- fluoromethyl)phenyl]-1,3,5- triazine-2,4-diamine | 616.6 | C30H26F6N6O2 |
| 7 | 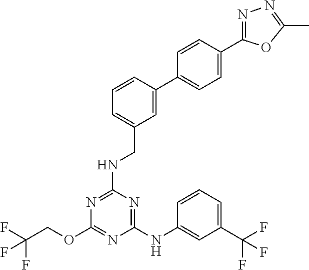 | N-{[4″-(5-methyl-1,3,4-oxadiazol- 2-yl)biphenyl-3-yl]methyl}-6- (2,2,2-trifluoroethoxy)-N′-[3-(tri- fluoromethyl)phenyl]-1,3,5- triazine-2,4-diamine | 601.5 | C28H21F6N7O2 |
| 8 | 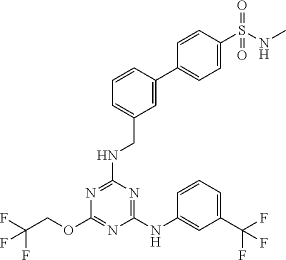 | N-methyl-3″-({[4-(2,2,2- trifluoroethoxy)-6-{[3- (trifluoromethyl)phenyl]amino}- 1,3,5-triazin-2- yl]amino}methyl)biphenyl-4- sulfonamide | 613.6 | C26H22F6N6O3S |
| 9 | 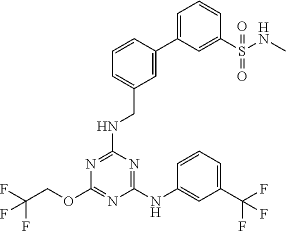 | N-methyl-3″-({[4-(2,2,2- trifluoroethoxy)-6-{[3- (trifluoromethyl)phenyl]amino}- 1,3,5-triazin-2- yl]amino}methyl)biphenyl-3- sulfonamide | 613.6 | C26H22F6N6O3S |
| 10 | 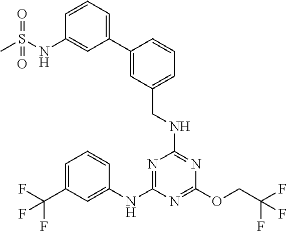 | N-[3″-({[4-(2,2,2-trifluoroethoxy)-6- {[3-(trifluoro- methyl)phenyl]amino}-1,3,5- triazin-2-yl]amino}- methyl)biphenyl-3- yl]methanesulfonamide | 613.6 | C26H22F6N6O3S |
| 11 | 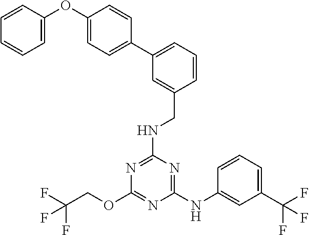 | N-[(4″-phenoxybiphenyl-3- yl)methyl]-6-(2,2,2- trifluoroethoxy)-N′-[3- (trifluoromethyl)phenyl]-1,3,5- triazine-2,4-diamine | 611.5 | C31H23F6N5O2 |
| 12 | 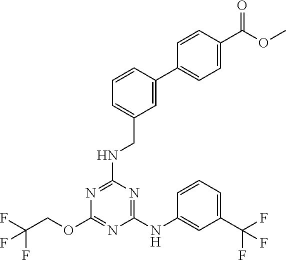 | methyl 3″-({[4-(2,2,2- trifluoroethoxy)-6-{[3- (trifluoromethyl)phenyl]amino}- 1,3,5-triazin-2- yl]amino}methyl)biphenyl-4- carboxylate | 577.5 | C27H21F6N5O3 |
| 13 | 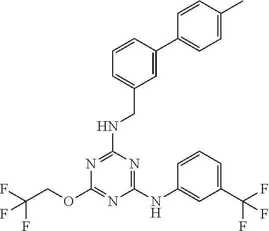 | N-[(4″-methylbiphenyl-3- yl)methyl]-6-(2,2,2- trifluoroethoxy)-N′-[3- (trifluoromethyl)phenyl]-1,3,5- triazine-2,4-diamine | 533.5 | C26H21F6N5O |
| 14 | 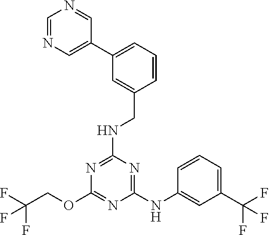 | N-[4-(pyridin-3-yl)benzyl]-6-(2,2,2- trifluoroethoxy)-N′-[3- (trifluoromethyl)phenyl]-1,3,5- triazine-2,4-diamine | 521.4 | C23H17F6N7O |
| 15 | 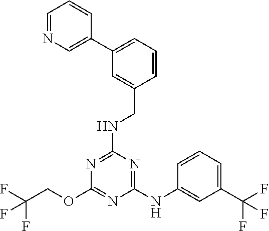 | 1-ethyl-N-[4-({[4-(2,2,2- trifluoroethoxy)-6-{[3- (trifluoromethyl)phenyl]amino}- 1,3,5-triazin-2- yl]amino}methyl)phenyl]-1H- pyrazole-3-carboxamide | 520.4 | C24H18F6N6O |
| 16 | 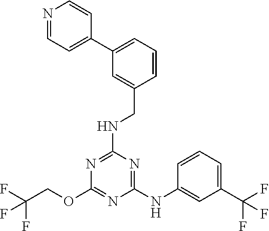 | 2-chloro-N-[4-({[4-(2,2,2- trifluoroethoxy)-6-{[3- (trifluoromethyl)phenyl]amino}- 1,3,5-triazin-2- yl]amino}methyl)phenyl]benzamide | 520.4 | C24H18F6N6O |

### Procedures and Analytical Data for Table 28

All entries were prepared by the methods for Library 25, Table 25.

### 1. N-[(4″-chloro-3″-fluorobiphenyl-3-yl)methyl]-6-(2,2,2-trifluoroethoxy)-N′-[3-(trifluoromethyl)phenyl]-1,3,5-triazine-2,4-diamine

1H NMR (DMSO, ppm) 4.80 m (2H), 4.98 m (2H), 7.26 d (1H), 7.44 m (4H), 7.6 m (4H), 7.98 bs (2H), 8.18 bs (1H), 9.65 bs (1H); LCMS: M+1=572

### 2. N-[(3″-fluorobiphenyl-3-yl)methyl]-6-(2,2,2-trifluoroethoxy)-N′-[3-(trifluoromethyl)phenyl]-1,3,5-triazine-2,4-diamine

1H NMR (DMSO, ppm) 4.72 m (2H), 4.98 m (2H), 7.18 t (1H), 7.40 m (8H), 7.64 s (1H), 7.86 bs (2H), 8.18 s (1H), 9.65 s (1H); LCMS: M+1=538

### 3. 6-(2,2,2-trifluoroethoxy)-N-{[3″-(trifluoromethyl)biphenyl-3-yl]methyl}-N′-[3-(trifluoromethyl)phenyl]-1,3,5-triazine-2,4-diamine

1H NMR (DMSO, ppm) 4.68 m (2H), 4.98 m (2H), 7.38 d (7H), 7.6 m (3H), 7.78 bs (2H), 8.18 bs (1H), 9.6 s (1H); LCMS: M+1=588

### 4. N-[(4″-fluorobiphenyl-3-yl)methyl]-6-(2,2,2-trifluoroethoxy)-N′-[3-(trifluoromethyl)phenyl]-1,3,5-triazine-2,4-diamine

1H NMR (DMSO, ppm) 4.70 m (2H), 4.98 m (2H), 7.28 d (1H), 7.42 m (3H), 7.60 d (1H), 7.70 bs (3H), 8.0 m (5H), 9.65 s (1H); LCMS: M+1=538

### 5. N-(4-fluorophenyl)-4-{[4-(2,2,2-trifluoroethoxy)-6-{[3-(trifluoromethyl)phenyl]amino}-1,3,5-triazin-2-yl]amino}benzenesulfonamide

LCMS: M+1=645.6 NMR 1H, DMSO-d6 δ, ppm: 1.57-1.70 m (8H); 3.49 bs (4H); 4.62 d (2H); 4.92 m (2H); 7.25-7.68 m (9H); 7.60-7.70 m (4H); 7.85-8.15 bm (3H); 9.60 bs (1H).

### 6. N-{[4′-(pyrrolidin-1-ylcarbonyl)-1,1′-biphenyl-3-yl]methyl}-6-(2,2,2-trifluoroethoxy)-N′-[3-(trifluoromethyl)phenyl]-1,3,5-triazine-2,4-diamine

1H NMR (DMSO, ppm) 1.17 m (4H), 3.52 m (4H), 4.64 m (2H), 4.98 m (2H), 7.38 m (4H), 7.58 d (3H), 7.64 d (3H), 8.0 m (2H), 8.18 s (1H), 9.70 s (1H); LCMS: M+1=617

### 7. N-{[4″-(5-methyl-1,3,4-oxadiazol-2-yl)biphenyl-3-yl]methyl}-6-(2,2,2-trifluoroethoxy)-N′-[3-(trifluoromethyl)phenyl]-1,3,5-triazine-2,4-diamine

1H NMR (DMSO, ppm) 2.70 s (3H), 4.78 d (2H), 4.98 m (2H), 7.3 d (1H), 7.42 m (3H), 7.6 d (1H), 7.72 s (1H), 7.82 d (2H), 8.0 m (5H), 9.56 bs (1H); LCMS: M+1=602

### 8. N-methyl-3″-{[4-(2,2,2-trifluoroethoxy)-6-{[3-(trifluoromethyl)phenyl]amino}-1,3,5-triazin-2-yl]amino}methyl)biphenyl-4-sulfonamide

1H NMR (DMSO, ppm) 2.5 s (3H), 4.74 m (2H), 4.98 m (2H), 7.16 s (1H), 7.30 d (1H), 7.44 m (3H), 7.58 d (1H), 7.72 s (1H), 7.8 m (3H), 7.78 m (2H), 8.18 s (1H), 9.64 s (1H); LCMS: M+1=613

### 9. N-methyl-3″-{[4-(2,2,2-trifluoroethoxy)-6-{[3-(trifluoromethyl)phenyl]amino}-1,3,5-triazin-2-yl]amino}methyl)biphenyl-3-sulfonamide

1H NMR (DMSO, ppm) 3.0 s (3H), 4.72 m (2H), 4.98 m (2H), 7.18 s (1H), 7.32 d (1H), 7.44 m (3H), 7.58 d (1H), 7.68 m (2H), 7.8 d (1H), 7.86 d (1H), 8.0 m (3H), 8.18 s (1H), 9.64 s (1H); LCMS: M+1=613

### 10. N-[3″-{[4-(2,2,2-trifluoroethoxy)-6-{[3-(trifluoromethyl)phenyl]amino}-1,3,5-triazin-2-yl]amino}methyl)biphenyl-3-yl]methanesulfonamide

1H NMR (DMSO, ppm) 2.98 s (3H), 4.70 m (2H), 4.98 m (2H), 7.24 d (1H), 7.40 m (8H), 7.6 s (1H), 8.0 m (2H), 8.18 s (1H), 9.65 s (1H); LCMS: M+1=613

### 11. N-[(4″-phenoxybiphenyl-3-yl)methyl]-6-(2,2,2-trifluoroethoxy)-N′-[3-(trifluoromethyl)phenyl]-1,3,5-triazine-2,4-diamine

1H NMR (DMSO, ppm) 4.70 d (2H), 4.98 m (2H), 7.04 m (4H), 7.18 t (1H), 7.4 m (7H), 7.6 m (3H), 7.98 bs (2H), 8.18 bs (1H), 9.7 bs (1H); LCMS: M+1=612

### 12. methyl 3″-{[4-(2,2,2-trifluoroethoxy)-6-{[3-(trifluoromethyl)phenyl]amino}-1,3,5-triazin-2-yl]amino}methyl)biphenyl-4-carboxylate

1H NMR (DMSO, ppm) 3.98 s (3H), 4.66 d (2H), 4.98 m (2H), 7.3 d (1H), 7.42 m (3H), 7.6 d (1H), 7.74 m (4H), 8.0 m (5H), 9.62 bs (1H); LCMS: M+1=578

### 13. N-[(4″-methylbiphenyl-3-yl)methyl]-6-(2,2,2-trifluoroethoxy)-N′-[3-(trifluoromethyl)phenyl]-1,3,5-triazine-2,4-diamine

1H NMR (DMSO, ppm) 2.3 s (3H), 4.70 d (2H), 4.98 m (2H), 7.3 m (5H), 7.44 m (4H), 7.6 s (1H), 7.88 bs (2H), 8.2 bs (1H), 9.8 bs (1H); LCMS: M+1=534.

14. N-[4-(pyridin-3-yl)benzyl]-6-(2,2,2-trifluoroethoxy)-N′-[3-(trifluoromethyl)phenyl]-1,3,5-triazine-2,4-diamine

LCMS: M+1=522.6 NMR 1H, DMSO-d6 δ, ppm: 4.63 d (2H); 4.93 m (2H); 7.28 d (1H); 7.49 m (3H); 7.60 d (1H); 7.79 s (1H); 7.85-8.15 bm (3H); 9.00 s (2H); 9.13 s (1H); 9.63 bs (1H).

### 15. 1-ethyl-N-[4-{[4-(2,2,2-trifluoroethoxy)-6-{[3-(trifluoromethyl)phenyl]amino}-1,3,5-triazin-2-yl]amino}methyl)phenyl]-1H-pyrazole-3-carboxamide

LCMS: M+1=521.6 NMR 1H, DMSO-d6 δ, ppm: 4.65 d (2H); 4.96 m (2H); 7.29 d (1H); 7.41-7.70 m (7H); 7.87-8.20 bm (4H); 8.61 d (2H); 8.88 s (1H); 9.63 bs (1H).

### 16. 2-chloro-N-[4-{[4-(2,2,2-trifluoroethoxy)-6-{[3-(trifluoromethyl)phenyl]amino}-1,3,5-triazin-2-yl]amino}methyl)phenyl]benzamide

LCMS: M+1=521.6 NMR 1H, DMSO-d6 δ, ppm: 4.63 d (2H); 4.95 m (2H); 7.28 d (1H); 7.42-7.69 m (7H); 7.75 s (1H); 7.79 s (1H); 7.85-8.15 bm (3H); 8.63 d (2H); 9.61 bs (1H).

### Generic Synthesis of Intermediates and Final Compounds for Table 29

(See Table 35 for other intermediates).

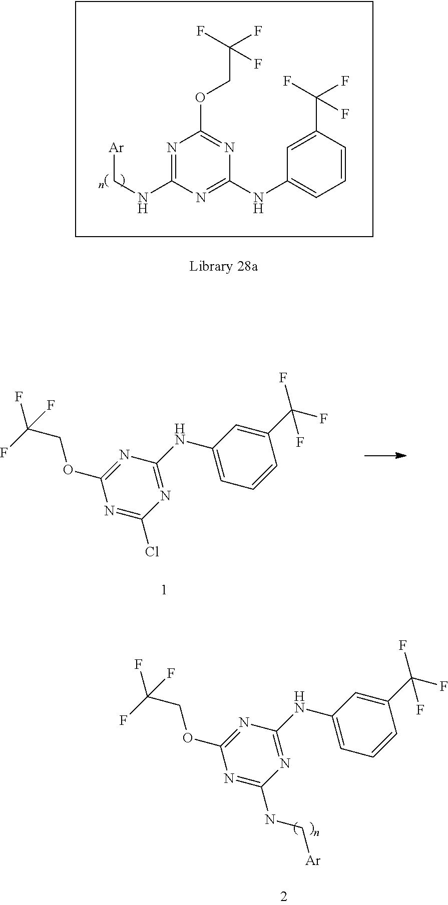

Preparation of 2: 0.0027 mol of compound 1 was dissolved in 100 ml of dry acetone, reaction mixture was cooled down to 0-5° C., then 0.0027 mol of amine in 50 ml of acetone were added dropwise, while the reaction temperature was maintained at 0-5° C. The reaction mixture was stirred at this temperature 0.5 h. Then, the reaction mixture was poured into 100 ml of ice water, and the resulting precipitate was filtered, washed with water, and lyophilized to afford final compounds

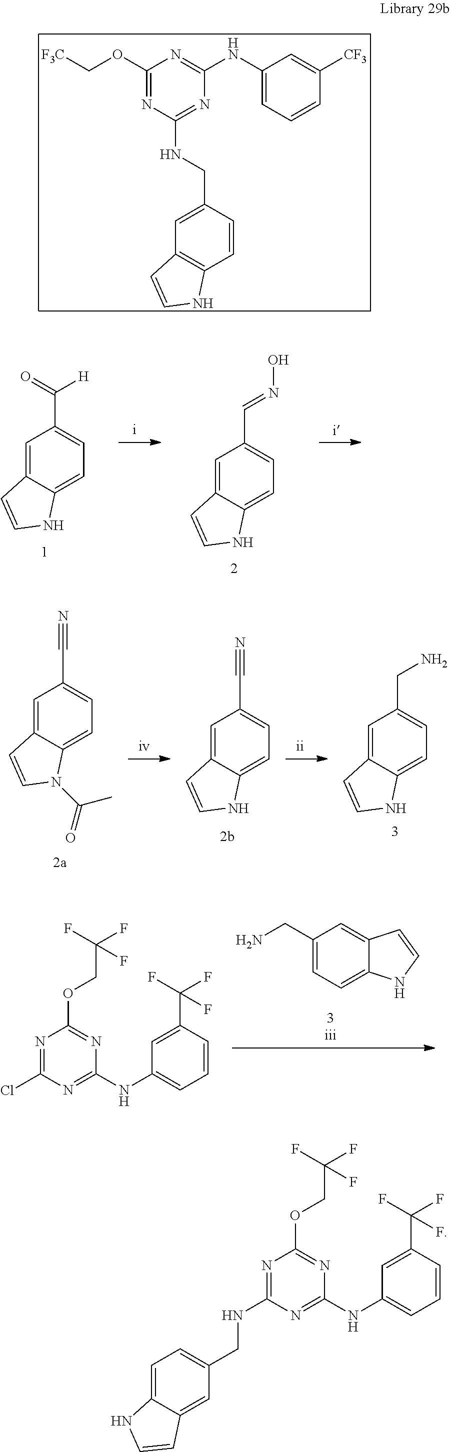

Intermediate 2a, 2b: To a solution of 1 (10 g) and ammonium formate (2 eq.) in pyridine (0.1 eq.) was added acetic anhydride (5 eq.) dropwise. The addition was exothermic. After the completion of the addition, the reaction mixture was refluxed overnight after which no starting material remained. The mixture was cooled and poured into ice-water. The resulting solution was filtered. The filtrate was collected, transferred to separatory funnel, and extracted with CHCl₃ (3×200 mL). The organic portions were combined, dried with Na₂SO₄, and filtered. The solvent was removed in vacuo producing 2a as a yellow oil. The residue was treated with 3% aq. HCl until the pH reached 3-4. The resulting white precipitate was collected by filtration, affording desired intermediate 2b. The solids were further recrystallized from MeOH giving analytically pure 2b.

Intermediate 3: To an ice-cold solution of LiAlH₄ (1.15 g, 1.8 eq.) in 50 mL of THF was added dropwise a solution of 2b in 50 mL of THF under Ar atmosphere. After the addition was completed, the reaction mixture was allowed to warm to r.t., and was stirred overnight. The reaction was quenched by a dropwise addition of 10% aq. NaOH under constant stirring in an ice-water bath. The product was extracted with ethyl acetate, dried with Na₂SO₄, and filtered. The filtrate was evaporated under reduced pressure, and the resulting residue was recrystallized from EtOAc/hexanes mixture (50/50, v/v) producing analytically pure 3.

Intermediate 3.1, Step iii: 250 mg of monochlorotriazine, 1.2 eq. of Et₃N and 1.2 of amine 3 were stirred in 5 mL of dioxane under reflux for 8 h. After cooling the solvent was removed under reduced pressure, and the dark residue was purified by column chromatography using 1% MeOH in CHCl₃.

TABLE 29

| En | Structure | IUPAC Name | MW | Formula |
|---|---|---|---|---|
| 1 | 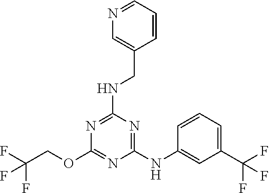 | N2-(pyridin-3-ylmethyl)-6-(2,2,2- trifluoroethoxy)-N4-(3- (trifluoromethyl)phenyl)-1,3,5-triazine-2,4- diamine | 444.3 | C₁₈H₁₄F₆N₆O |
| 2 | 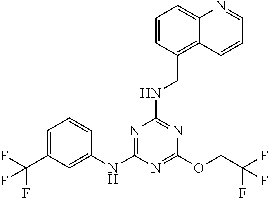 | N-(quinolin-5-ylmethyl)-6-(2,2,2- trifluoroethoxy)-N′-[3- (trifluoromethyl)phenyl]-1,3,5-triazine-2,4- diamine | 494.4 | C₂₂H₁₆F₆N₆O |
| 3 | 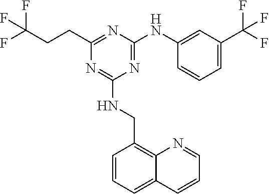 | N-(quinolin-8-ylmethyl)-6-(2,2,2- trifluoroethoxy)-N′-[3- (trifluoromethyl)phenyl]-1,3,5-triazine-2,4- diamine | 494.4 | C₂₂H₁₆F₆N₆O |
| 4 | 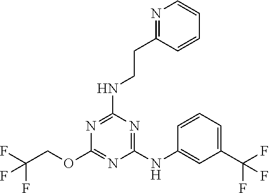 | N-[2-(pyridin-2-yl)ethyl]-6-(2,2,2- trifluoroethoxy)-N′-[3- (trifluoromethyl)phenyl]-1,3,5-triazine-2,4- diamine | 458.4 | C₁₉H₁₆F₆N₆O |
| 5 | 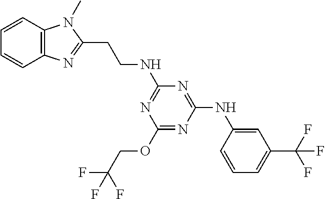 | N-[2-(1-methyl-1H-benzimidazol-2- yl)ethyl]-6-(2,2,2-trifluoroethoxy)-N′-[3 (trifluoromethyl)phenyl]-1,3,5-triazine-2,4- diamine | 511.4 | C22H19F6N7O |
| 6 | 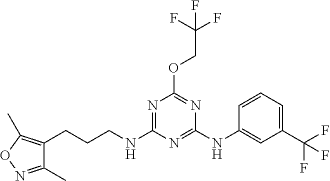 | N-[3-(3,5-dimethylisoxazol-4-yl)propyl]-6- (2,2,2-trifluoroethoxy)-N′-[3- (trifluoromethyl)phenyl]-1,3,5-triazine-2,4- diamine | 490.4 | C₂₀H₂₀F₆N₆O₂ |
| 7 | 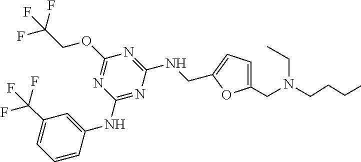 | N-[(5-{[butyl(ethyl)amino]methyl}furan-2- yl)methyl]-6-(2,2,2-trifluoroethoxy)-N′-[3- (trifluoromethyl)phenyl]-1,3,5-triazine-2,4- diamine | 546.5 | C₂₄H₂₈F₆N₆O₂ |
| 8 | 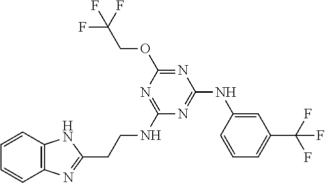 | N-[2-(1H-benzimidazol-2-yl)ethyl]-6- (2,2,2-trifluoroethoxy)-N′-[3- (trifluoromethyl)phenyl]-1,3,5-triazine-2,4- diamine | 497.4 | C₂₁H₁₇F₆N₇O |
| 9 | 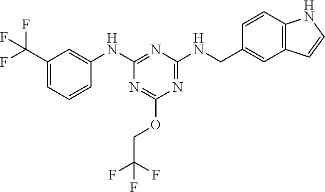 | N2-((1H-indol-5-yl)methyl)-6-(2,2,2- trifluoroethoxy)-N4-(3- (trifluoromethyl)phenyl)-1,3,5-triazine-2,4- diamine | 482.4 | C₂₁H₁₆F₆N₆O |
| 10 | 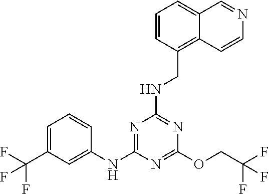 | N2-(isoquinolin-5-ylmethyl)-6-(2,2,2- trifluoroethoxy)-N4-(3- (trifluoromethyl)phenyl)-1,3,5-triazine-2,4- diamine | 494.4 | C₂₂H₁₆F₆N₆O |
| 11 | 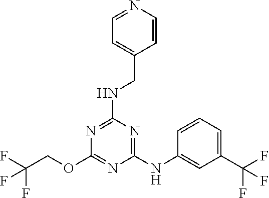 | N2-(pyridin-4-ylmethyl)-6-(2,2,2- trifluoroethoxy)-N4-(3- (trifluoromethyl)phenyl)-1,3,5-triazine-2,4- diamine | 444.3 | C₁₈H₁₄F₆N₆O |
| 12 | 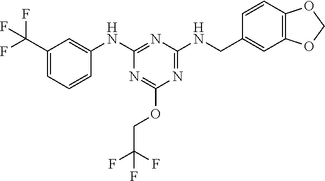 | N2-(benzo[d][1,3]dioxol-5-ylmethyl)-6- (2,2,2-trifluoroethoxy)-N4-(3- (trifluoromethyl)phenyl)-1,3,5-triazine-2,4- diamine | 487.4 | C₂₀H₁₅F₆N₅O₃ |
| 13 | 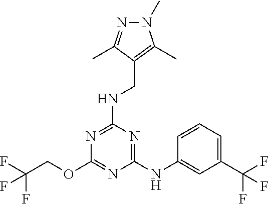 | 6-(2,2,2-trifluoroethoxy)-N2-(3- (trifluoromethyl)phenyl)-N4-((1,3,5- trimethyl-1H-pyrazol-4-yl)methyl)-1,3,5- triazine-2,4-diamine | 475.4 | C19H19F6N7O |
| 14 | 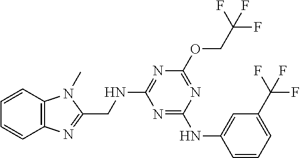 | N2-((1-methyl-1H-benzo[d]imidazol-2- yl)methyl)-6-(2,2,2-trifluoroethoxy)-N4-(3- (trifluoromethyl)phenyl)-1,3,5-triazine-2,4- diamine | 497.4 | C₂₁H₁₇F₆N₇O |

### Procedures and Analytical Data for Table 29.

Entry 9 is a Library 29b analog. Entries 1 to 8, and 10 to 14 are from Library 29a.

### 1. N-2-(pyridin-3-ylmethyl)-6-(2,2,2-trifluoroethoxy)-N-4-(3-(trifluoromethyl)phenyl)-1,3,5-triazine-2,4-diamine

LCMS: M+1=445.6; 1H NMR, DMSO-d6 δ, ppm: 4.59 d (2H); 4.88 m (2H); 7.25 t (2H); 7.43 t (1H); 7.71 d (1H); 7.88 m (2H); 8.14 s (1H); 8.43 d (1H); 8.57 s (1H); 9.54 bs (1H).

### 2. N-(quinolin-5-ylmethyl)-6-(2,2,2-trifluoroethoxy)-N′-[3-(trifluoromethyl)phenyl]-1,3,5-triazine-2,4-diamine

LCMS: M+1=494.4; 1H NMR, DMSO-d6 δ, ppm: 4.9 (4H, m); 7.5 (5H, m); 7.9 (4H, m); 8.6 (1H, d); 8.9 (1H, q); 9.6 (1H, s).

### 3. N-(quinolin-8-ylmethyl)-6-(2,2,2-trifluoroethoxy)-N′-[3-(trifluoromethyl)phenyl]-1,3,5-triazine-2,4-diamine

LCMS: M+1=494.4; 1H NMR, DMSO-d6 δ, ppm: 4.9 (2H, q); 5.2 (2H, d); 7.5 (5H, m); 7.2 (2H, m); 7.7 (6H, m); 8.6 (1H, d); 8.1 (1H, s); 8.4 (1H, d); 8.9 (1H, m); 9.6 (1H, s).

### 4. N-[2-(pyridin-2-yl)ethyl]-6-(2,2,2-trifluoroethoxy)-N′-[3-(trifluoromethyl)phenyl]-1,3,5-triazine-2,4-diamine

LCMS: M+1=458.3; 1H NMR, DMSO-d6 δ, ppm: 3.0 (2H, t); 3.7 (2H, q); 4.9 (2H, q); 7.3 (5H, m); 7.6 (1H, t); 7.9 (1H, d); 8.2 (1H, s); 8.5 (1H, d); 9.6 (1H, s).

### 5. N-[2-(1-methyl-1H-benzimidazol-2-yl)ethyl]-6-(2,2,2-trifluoroethoxy)-N′-[3-(trifluoromethyl)phenyl]-1,3,5-triazine-2,4-diamine

LCMS: M+1=511.4; 1H NMR, DMSO-d6 δ, ppm: 3.2 (2H, t); 3.7 (3H, s); 3.9 (2H, d); 4.9 (2H, q); 7.15 (2H, m); 7.3 (1H, d); 7.5 (4H, m); 7.9 (1H, d); 8.2 (1H, s); 9.6 (1H, s).

### 6. N-[3-(3,5-dimethylisoxazol-4-yl)propyl]-6-(2,2,2-trifluoroethoxy)-N′-[3-(trifluoromethyl)phenyl]-1,3,5-triazine-2,4-diamine

LCMS: M+1=490.4; 1H NMR, DMSO-d6 δ, ppm: 1.8 (2H, m); 2.1 (3H, s); 2.2 (3H, s); 2.3 (2H, t); 3.35 (2H, q); 4.9 (2H, q); 7.4 (3H, m); 8.0 (2H, m); 9.6 (1H, s).

### 7. N-[(5-{[butyl(ethyl)amino]methyl}furan-2-yl)methyl]-6-(2,2,2-trifluoroethoxy)-N′-[3-(trifluoromethyl)phenyl]-1,3,5-triazine-2,4-diamine

LCMS: M+1=546.5; 1H NMR, DMSO-d6 δ, ppm: 0.9 (6H, m); 1.3 (5H, m); 2.4 (3H, m); 3.5 (2H, s); 4.5 (2H, d); 4.9 (2H, q); 6.1 (2H, d); 7.3 (1H, d); 7.5 (1H, t); 7.75 (1H, s); 7.96 (1H, d); 8.2 (1H, s); 9.6 (1H, s).

### 8. N-[2-(1H-benzimidazol-2-yl)ethyl]-6-(2,2,2-trifluoroethoxy)-N′-[3-(trifluoromethyl)phenyl]-1,3,5-triazine-2,4-diamine

LCMS: M+1=498.1; NMR 1H, DMSO-d6 δ, ppm: 3.85 m (2H); 4.95 m (2H); 7.06-7.16 m (2H); 7.3 d (1H); 7.38-7.60 m (4H); 7.97 d (1H); 8.1-8.24 m (2H); 9.6 s (1H); 11.95 s (1H).

### 9. N-2-((1H-indol-5-yl)methyl)-6-(2,2,2-trifluoroethoxy)-N-4-(3-(trifluoromethyl)phenyl)-1,3,5-triazine-2,4-diamine

1H NMR (DMSO-d6, 90° C., ppm): d=4.64 d (2H, CH2), 4.94 q (2H, CH2), 6.36 s (1H, Ar), 7.10 d (1H, Ar), 7.21-7.38 m (3H, Ar), 7.43-7.54 m (2H, Ar), 7.83 t (1H, Ar), 7.95 d (1H, Ar), 8.19 s (1H, Ar), 9.56 s (1H, NH), 10.68 s (1H, NH). LC-MS [M+1]: calc'd: 483.4; obs'd: 483.2.

### 10. N-2-(isoquinolin-5-ylmethyl)-6-(2,2,2-trifluoroethoxy)-N-4-(3-(trifluoromethyl)phenyl)-1,3,5-triazine-2,4-diamine

LCMS: M+1=495.6 NMR 1H, DMSO-d6 δ, ppm: 4.98 m (4H); 7.25 m (2H); 7.43 m (1H); 7.60 t (1H); 7.72 m (2H); 8.00-8.20 m (4H); 8.57 d (1H); 9.20 s (1H); 9.70 bs (1H).

### 11. N-2-(pyridin-4-ylmethyl)-6-(2,2,2-trifluoroethoxy)-N-4-(3-(trifluoromethyl)phenyl)-1,3,5-triazine-2,4-diamine

LCMS: M+1=445.6; 1H NMR, DMSO-d6 δ, ppm: 4.59 t (2H); 4.92 m (2H); 7.28 t (3H); 7.48 m (1H); 7.80-8.10 m (3H); 8.48 t (2H); 9.58 bs (1H).

### 12. N-2-(benzo[d][1,3]dioxol-5-ylmethyl)-6-(2,2,2-trifluoroethoxy)-N-4-(3-(trifluoromethyl)phenyl)-1,3,5-triazine-2,4-diamine

LCMS: M+1=488.6 NMR 1H, DMSO-d6 δ, ppm: 4.59 d (2H); 4.88 m (2H); 5.95 s (2H); 6.80 m (3H); 7.25 d (2H); 7.43 t (1H); 7.71 t (1H); 7.82-8.20 m (2H); 9.54 bs (1H).

### 13. 6-(2,2,2-trifluoroethoxy)-N-2-(3-(trifluoromethyl)phenyl)-N-4-((1,3,5-trimethyl-1H-pyrazol-4-yl)methyl)-1,3,5-triazine-2,4-diamine

LCMS: M+1=476.6 NMR 1H, DMSO-d6 δ, ppm: 2.10 s (3H); 2.24 s (3H); 3.66 s (3H); 4.59 d (2H); 4.88 m (2H); 7.25 m (2H); 7.43 t (1H);); 7.82-8.20 m (2H); 9.54 bs (1H).

### 14. N-2-((1-methyl-1H-benzo[d]imidazol-2-yl)methyl)-6-(2,2,2-trifluoroethoxy)-N-4-(3-(trifluoromethyl)phenyl)-1,3,5-triazine-2,4-diamine

LCMS: M+1=498.6 NMR 1H, DMSO-d6 δ, ppm: 4.85 d (2H); 4.97 m (2H); 6.80 m (3H); 7.20 m (3H); 7.50 m (3H); 7.82-8.20 m (3H); 9.80 bs (1H).

### Generic Synthesis of Intermediates and Final Compounds in Table 30

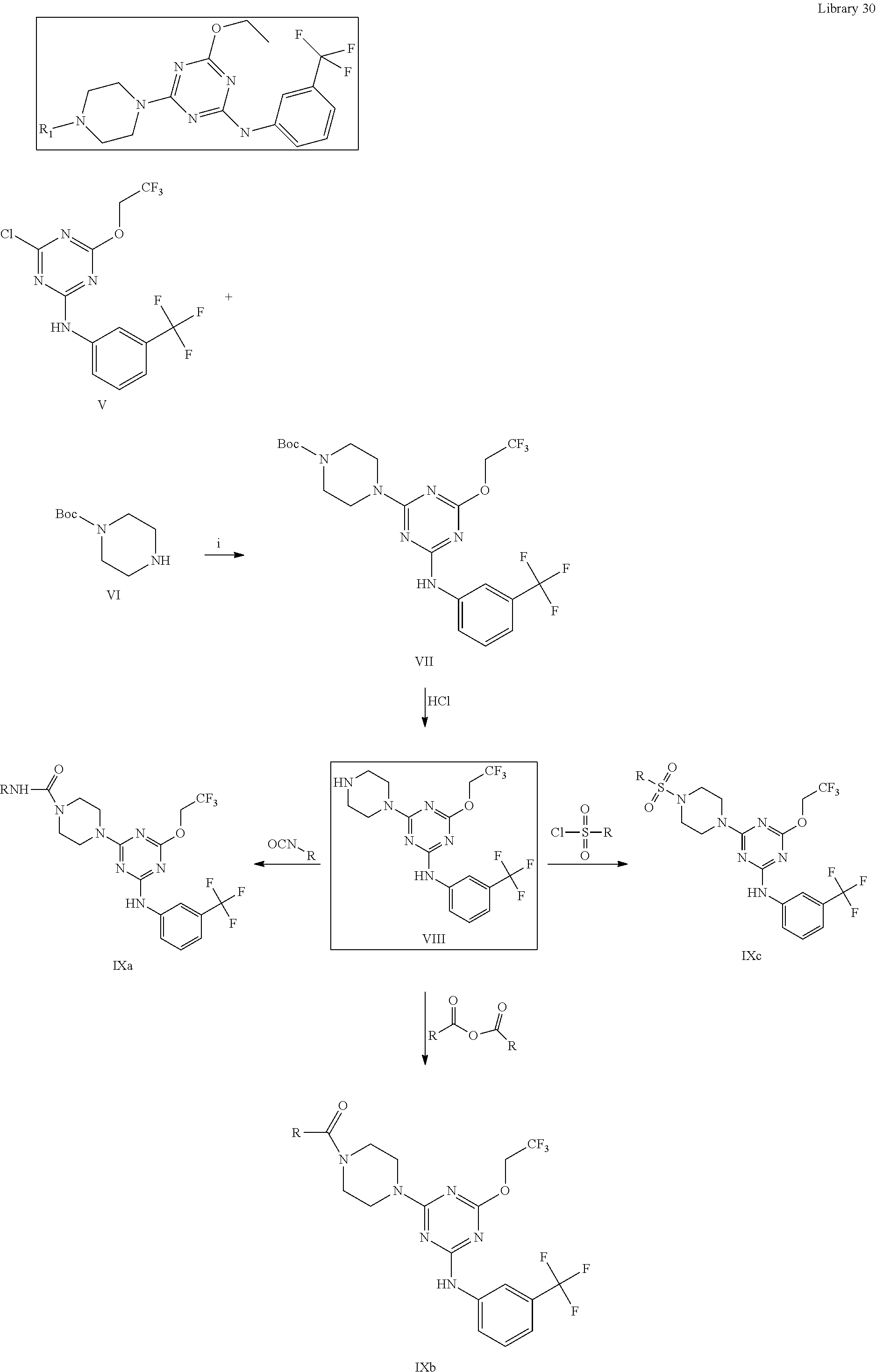

Preparation of tert-butyl 4-(2,2,2-trifluoroethoxy)-6-{[3-(trifluoromethyl)phenyl]amino}-1,3,5-triazin-2-yl)piperazine-1-carboxylate VII. BOC-piperazine VI (2.50 g) was added portion-wise to a stirred solution of trifluoroethoxyanilinotriazine V (5 g) in 200 ml of dioxane in the presence of triethylamine (1.63 g). After 7 h with stirring at r.t., the reaction mixture was checked by LCMS, the solvent was removed under reduced pressure, and the residue was treated with water and extracted with CH₂Cl₂. The organic phase was dried over sodium sulphate, filtered and purified by chromatography on silica gel with hexane:ethylacetate mixture (3:1) as eluent. The target fractions were concentrated and washed with hexane providing intermediate VII as a white solid. Yield 50% (3.5 g).

Preparation of 4-(4-(2,2,2-trifluoroethoxy)-6-{[3-(trifluoromethyl)phenyl]amino}-1,3,5-triazin-2-yl)piperazine VIII. A suspension of compound VII (0.6 g) was stirred for 6 h. at rt in 6 N HCl in ethanol-ethyl acetate, then concentrated and diluted with water. The aqueous solution was basified with aqueous NaOH solution to pH 10. The precipitate formed was filtered, washed with water and air-dried. Compound VIII was obtained as a white solid in 80% yield (0.3 g).

Preparation of 4-(4-(2,2,2-trifluoroethoxy)-6-{[3-(trifluoromethyl)phenyl]amino}-1,3,5-triazin-2-yl)N-substituted-piperazinecarboxamide IXa. A solution of 4-(4-ethoxy-6-{[3-(trifluoro-methyl)-phenyl]-anilino}-1,3,5-triazin-2-yl)piperazine VIII (10 mmol) in 2 ml of dioxane was treated with an appropriate isocyanate (10 mmol) and triethylamine (11 mmol). This mixture was stirred at rt for 3 h, then poured into water. The formed precipitate IXa was purified via silica gel chromatography with appropriate eluent (hexanes-ethyl acetate or methylene chloride-ethanol).

Preparation of 4-(4-(2,2,2-trifluoroethoxy)-6-{[3-(trifluoromethyl)phenyl]amino}-1,3,5-triazin-2-yl)N-substituted-piperazineamide IXb. A solution of 4-(4-ethoxy-6-{[3-(trifluoro-methyl)-phenyl]-anilino}-1,3,5-triazin-2-yl)piperazine VIII (12 mmol) in dioxane (2 ml) was treated with acyl anhydride (10 mmol) in the presence of Et₃N (12 mmol). The reaction mixture was stirred at rt for 3 h and poured into saturated aqueous brine solution. The precipitate IXb was filtered, washed with water and purified via silica gel chromatography with appropriate eluent (hexanes-ethyl acetate or methylene chloride-ethanol).

Preparation of 4-(4-(2,2,2-trifluoroethoxy)-6-{[3-(trifluoromethyl)phenyl]amino}-1,3,5-triazin-2-yl)N-substituted-piperazinesulfonamide IXc. 4-(4-ethoxy-6-{[3-(trifluoro-methyl)-phenyl]-anilino}-1,3,5-triazin-2-yl)piperazine VIII (10 mmol) was dissolved in 2 ml of dioxane and treated successively with an appropriate sulfonyl chloride (11.5 mmol) and triethylamine (11 mmol). The precipitate IXc was filtered, washed with water, and then washed with 3% aqueous HCl to afford the final compounds.

TABLE 30

| En | Structure | IUPAC Name | MW | Formula |
|---|---|---|---|---|
| 1 | 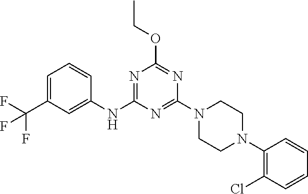 | 4-[4-(2-chlorophenyl)piperazin- 1-yl]-6-ethoxy-N-[3- (trifluoromethyl)phenyl]-1,3,5- triazin-2-amine | 478.9 | C22H22ClF3N6O |
| 2 | 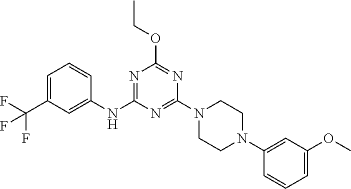 | 4-ethoxy-6-[4-(3- methoxyphenyl)piperazin-1-yl] N-[3-(trifluoromethyl)phenyl]- 1,3,5-triazin-2-amine | 474.5 | C23H25F3N6O2 |
| 3 | 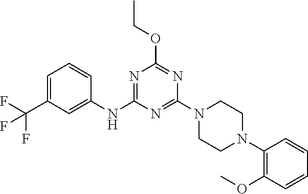 | 4-ethoxy-6-[4-(2- methoxyphenyl)piperazin-1-yl]- N-[3-(trifluoromethyl)phenyl]- 1,3,5-triazin-2-amine | 474.5 | C23H25F3N6O2 |
| 4 | 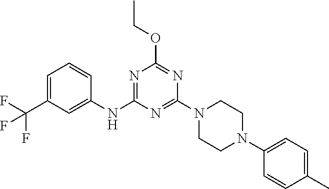 | 4-ethoxy-6-(4-p-tolylpiperazin-1- yl)-N-(3-(trifluoromethyl)phenyl)- 1,3,5-triazin-2-amine | 458.5 | C23H25F3N6O |
| 5 | 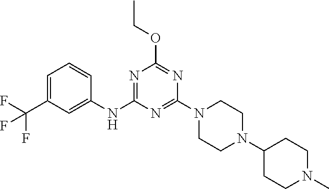 | 4-ethoxy-6-[4-(1- methylpiperidin-4-yl)piperazin-1- yl]-N-[3-(trifluoromethyl)-phenyl]- 1,3,5-triazin-2-amine | 465.5 | C22H30F3N7O |
| 6 | 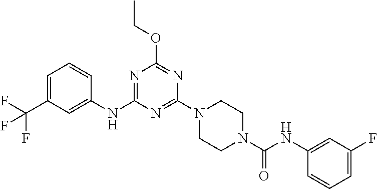 | 4-(4-ethoxy-6-{[3- (trifluoromethyl)-phenyl]amino}- 1,3,5-triazin-2-yl)-N-(3- fluorophenyl)piperazine-1- carboxamide | 505.5 | C23H23F4N7O2 |
| 7 | 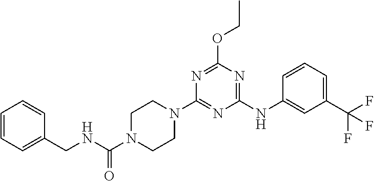 | N-benzyl-4-(4-ethoxy-6-{[3- (trifluoromethyl)phenyl]amino}- 1,3,5-triazin-2-yl)piperazine-1- carboxamide | 501.5 | C24H26F3N7O2 |
| 8 | 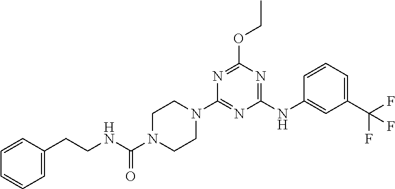 | 4-(4-ethoxy-6-{[3- (trifluoromethyl)-phenyl]amino}- 1,3,5-triazin-2-yl)-N-(2- phenylethyl)piperazine-1- carboxamide | 515.5 | C25H28F3N7O2 |
| 9 | 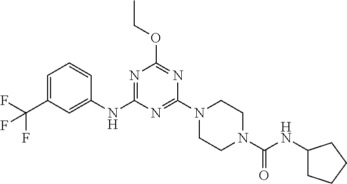 | N-cyclopentyl-4-(4-ethoxy-6-{[3- (trifluoromethyl)phenyl]amino}- 1,3,5-triazin-2-yl)piperazine-1- carboxamide | 479.5 | C22H28F3N7O2 |
| 10 | 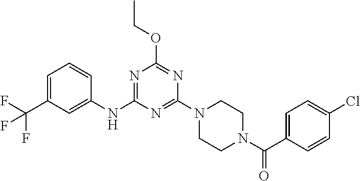 | (4-chlorophenyl)[4-(4-ethoxy-6- {[3- (trifluoromethyl)phenyl]amino}- 1,3,5-triazin-2-yl)piperazin-1- yl]methanone | 506.9 | C23H22ClF3N6O2 |
| 11 | 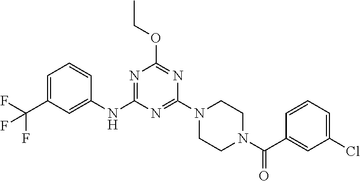 | (3-chlorophenyl)[4-(4-ethoxy-6- {[3- (trifluoromethyl)phenyl]amino}- 1,3,5-triazin-2-yl)piperazin-1- yl]methanone | 506.9 | C23H22ClF3N6O2 |
| 12 | 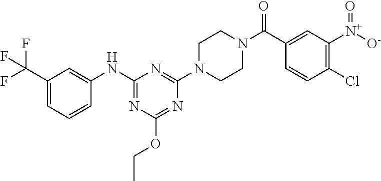 | (4-chloro-3-nitrophenyl)[4-(4- ethoxy-6-{[3- (trifluoromethyl)phenyl]amino}- 1,3,5-triazin-2-yl)piperazin-1- yl]methanone | 551.9 | C23H21ClF3N7O4 |
| 13 | 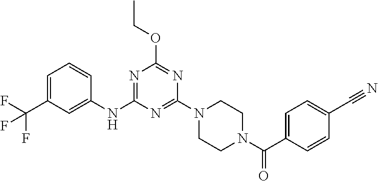 | 4-{[4-(4-ethoxy-6-{[3- (trifluoromethyl)phenyl]amino}- 1,3,5-triazin-2-yl)piperazin-1- yl]carbonyl}benzonitrile | 497.5 | C24H22F3N7O2 |
| 14 | 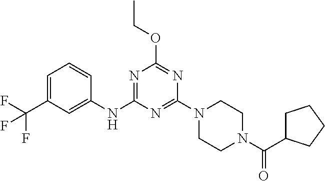 | cyclopentyl[4-(4-ethoxy-6-{[3- (trifluoromethyl)phenyl]amino}- 1,3,5-triazin-2-yl)piperazin-1- yl]methanone | 464.5 | C22H27F3N6O2 |
| 15 | 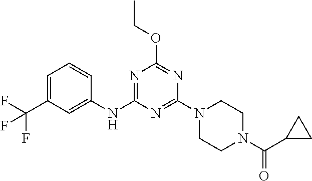 | cyclopropyl[4-(4-ethoxy-6-{[3- (trifluoromethyl)phenyl]amino}- 1,3,5-triazin-2-yl)piperazin-1- yl]methanone | 436.4 | C20H23F3N6O2 |
| 16 | 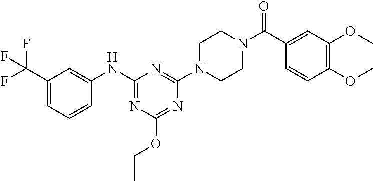 | (3,4-dimethoxyphenyl)[4-(4- ethoxy-6-{[3- (trifluoromethyl)phenyl]amino}- 1,3,5-triazin-2-yl)piperazin-1- yl]methanone | 532.5 | C25H27F3N6O4 |
| 17 | 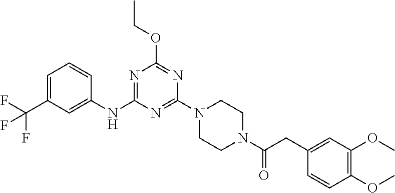 | 2-(3,4-dimethoxyphenyl)-1-[4-(4- ethoxy-6-{[3- (trifluoromethyl)phenyl]amino}- 1,3,5-triazin-2-yl)piperazin-1- yl]ethanone | 546.5 | C26H29F3N6O4 |
| 18 | 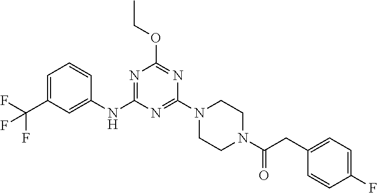 | 1-[4-(4-ethoxy-6-{[3- (trifluoromethyl)phenyl]amino}- 1,3,5-triazin-2-yl)piperazin-1-yl]- 2-(4-fluorophenyl)ethanone | 504.5 | C24H24F4N6O2 |
| 19 | 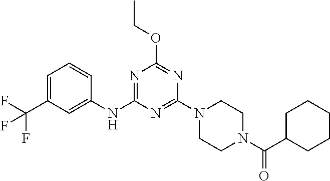 | cyclohexyl[4-(4-ethoxy-6-{[3- (trifluoromethyl)phenyl]amino}- 1,3,5-triazin-2-yl)piperazin-1- yl]methanone | 478.5 | C23H29F3N6O2 |
| 20 | 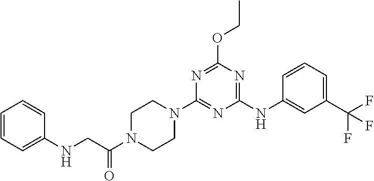 | 1-[4-(4-ethoxy-6-{[3- (trifluoromethyl)-phenyl]amino}- 1,3,5-triazin-2-yl)piperazin-1-yl]- 2-(phenylamino)ethanone | 501.5 | C24H26F3N7O2 |
| 21 | 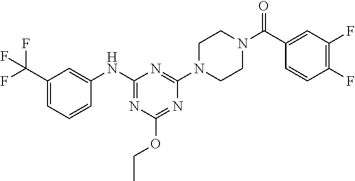 | (3,4-difluorophenyl)[4-(4-ethoxy- 6-{[3- (trifluoromethyl)phenyl]amino}- 1,3,5-triazin-2-yl)piperazin-1- yl]methanone | 508.4 | C23H21F5N6O2 |
| 22 | 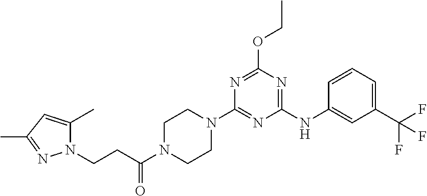 | 3-(3,5-dimethyl-1H-pyrazol-1-yl)-1- [4-(4-ethoxy-6-{[3- (trifluoromethyl)phenyl]amino}- 1,3,5-triazin-2-yl)piperazin-1- yl]propan-1-one | 518.5 | C24H29F3N8O2 |
| 23 | 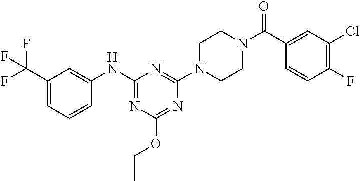 | (3-chloro-4-fluorophenyl)[4-(4- ethoxy-6-{[3- (trifluoromethyl)phenyl]amino}- 1,3,5-triazin-2-yl)piperazin-1- yl]methanone | 524.9 | C23H21ClF4N6O2 |
| 24 | 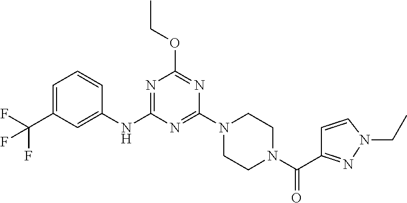 | [4-(4-ethoxy-6-{[3- (trifluoromethyl)phenyl]-amino}- 1,3,5-triazin-2-yl)piperazin-1- yl](1-ethyl-1H-pyrazol-3- yl)methanone | 490.5 | C22H25F3N8O2 |
| 25 | 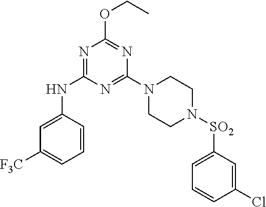 | 4-{4-[(3- chlorophenyl)sulfonyl]piperazin- 1-yl}-6-ethoxy-N-[3- (trifluoromethyl)phenyl]-1,3,5- triazin-2-amine | 544.0 | C22H22ClF3N6O3S |
| 26 | 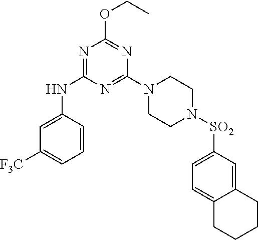 | 4-ethoxy-6-[4-(5,6,7,8- tetrahydronaphthalen-2- ylsulfonyl)piperazin-1-yl]-N-[3- (trifluoromethyl)phenyl]-1,3,5- triazin-2-amine | 563.6 | C26H29F3N6O3S |

### Procedures and Analytical Data for Table 30.

Entries 1-26 were prepared by the methods for library 30.

### 1. 4-[4-(2-chlorophenyl)piperazin-1-yl]-6-ethoxy-N-[3-(trifluoromethyl)phenyl]-1,3,5-triazin-2-amine

LCMS: M+1=478.9; NMR 1H, DMSO-d6 δ, ppm: 1.3 t (3H); 3.05 m (4H); 3.95 m (4H); 4.35 m (2H); 7.05 t (1H); 7.28 d (1H); 7.30 t (2H); 7.44 d (1H); 7.54 t (1H); 7.90 d (1H); 8.30 s (1H); 9.90 S (1H).

### 2. 4-ethoxy-6-[4-(3-methoxyphenyl)piperazin-1-yl]-N-[3-(trifluoromethyl)phenyl]-1,3,5-triazin-2-amine

LCMS: M+1=474.4; NMR CDCl3, ppm: 1.40 t (3H); 3.25 m (4H); 3.60 s (3H); 4.00 m (4H); 4.45 m (2H); 6.45-6.60 m (3H); 7.10-7.35 m (4H); 7.40-7.60 m (2H); 8.30 s (1H).

### 3. 4-ethoxy-6-[4-(2-methoxyphenyl)piperazin-1-yl]-N-[3-(trifluoromethyl)phenyl]-1,3,5-triazin-2-amine

LCMS: M+1=474.4; 1H NMR, DMSO-d6 δ, ppm: 1.3 t (3H); 3.05 m (4H); 3.80 s (3H); 3.95 m (4H); 4.35 m (2H); 6.8-7.10 m (4H); 7.30 d (1H); 7.50 t (2H); 7.90 d (1H); 7.54 t (1H); 7.90 d (1H); 9.90 S (1H).

### 4. 4-ethoxy-6-(4-p-tolylpiperazin-1-yl)-N-(3-(trifluoromethyl)phenyl)-1,3,5-triazin-2-amine

LCMS: M+1=458.4; 1H NMR CDCl3, ppm: 1.40 t (3H); 2.30 s (3H); 3.20 m (4H); 4.00 m (4H); 4.45 m (2H); 6.90 d (2H); 7.12 d (2H); 7.28 s (1H); 7.32 m (2H): 7.44 t (1H); 7.54 d (1H); 8.30 s (1H).

### 5. 4-ethoxy-6-[4-(1-methylpiperidin-4-yl)piperazin-1-yl]-N-[3-(trifluoromethyl)phenyl]-1,3,5-triazin-2-amine

LCMS: M+1=465.5; 1H NMR, DMSO-d6 δ, ppm: 1.20-1.50 m (6H); 1.70 t (2H); 1.90 t (2H); 2.20 m (4H); 2.85 t (2H); 3.75 m (4H); 4.35 m (2H); 7.30 d (1H); 7.42 t (1H); 7.84 d (1H); 8.34 s (1H); 9.85 s (1H).

### 6. 4-(4-ethoxy-6-{[3-(trifluoromethyl)phenyl]amino}-1,3,5-triazin-2-yl)-N-(3-fluorophenyl)piperazine-1-carboxamide

LCMS: M+1=505.4; 1H NMR, DMSO-d6 δ, ppm: 1.35 t (3H); 3.60 m (4H); 3.85 m (4H); 4.35 m (2H); 6.70 t (1H); 7.15-7.30 m (3H); 7.35-7.50 m (2H); 7.85 d (1H); 8.35 s (1H); 8.75 s (1H); 9.85 s (1H).

### 7. N-benzyl-4-(4-ethoxy-6-{[3-(trifluoromethyl)phenyl]amino}-1,3,5-triazin-2-yl)piperazine-1-carboxamide

LCMS: M+1=501.5; 1H NMR, DMSO-d6 δ, ppm: 1.35 t (3H); 3.45 m (4H); 3.85 m (4H); 4.35 m (2H); 4.40 m (2H); 7.05-7.35 m (7H); 7.45 t (1H); 7.63 d (1H); 8.35 s (1H); 9.80 (1H).

### 8. 4-(4-ethoxy-6-{[3-(trifluoromethyl)phenyl]amino}-1,3,5-triazin-2-yl)-N-(2-phenylethyl)piperazine-1-carboxamide

LCMS: M+1=515.5; 1H NMR, DMSO-d6 δ, ppm: 1.35 t (3H); 2.75 t (2H); 3.45 m (4H); 3.30 t (2H); 3.40 m (4H); 3.75 m (4H); 4.35 m (2H); 6.65 s (1H); 7.15-7.40 m (6H); 7.45 t (1H); 7.80 d (1H); 8.35 s (1H); 9.80 s (1H).

### 9. N-cyclopentyl-4-(4-ethoxy-6-{[3-(trifluoromethyl)phenyl]amino}-1,3,5-triazin-2-yl)piperazine-1-carboxamide

LCMS: M+1=479.5; 1H NMR, DMSO-d6 δ, ppm: 1.30-1.70 m (10H); 2.80 m (2H); 3.35 m (4H); 3.85 m (4H); 3.90 m (1H); 4.35 m (2H); 6.25 d (1H); 7.24 d (1H); 7.46 t (1H); 7.80 d (1H); 8.34 s (1H); 9.80 s (1H).

### 10. (4-chlorophenyl)[4-(4-ethoxy-6-{[3-(trifluoromethyl)phenyl]amino}-1,3,5-triazin-2-yl)piperazin-1-yl]methanone

LCMS: M+1=506.9; 1H NMR, DMSO-d6 δ, ppm: 1.35 t (3H); 3.60 m (4H); 4.90 m (4H); 4.35 m (2H); 7.28 d (1H); 7.42-7.58 m (5H); 7.88 d (1H); 8.24 s (1H); 9.50 s (1H).

### 11. (3-chlorophenyl)[4-(4-ethoxy-6-{[3-(trifluoromethyl)phenyl]amino}-1,3,5-triazin-2-yl)piperazin-1-yl]methanone

LCMS: M+1=506.9; 1H NMR, DMSO-d6 δ, ppm: 1.35 t (3H); 3.60 m (4H); 3.90 m (4H); 4.40 m (2H); 7.30 d (1H); 7.40 d (1H); 7.44-7.58 m (4H); 7.88 d (1H); 8.24 s (1H); 9.50 (1H).

### 12. (4-chloro-3-nitrophenyl)[4-(4-ethoxy-6-{[3-(trifluoromethyl)phenyl]amino}-1,3,5-triazin-2-yl)piperazin-1-yl]methanone

LCMS: M+1=372.6; 1H NMR (DMSO-d6, 90° C., ppm): =4.98 m (2H), 7.42 m (1H), 7.58 t (1H), 7.95 d (1H), 8.48 s (1H), 11.01 s (1H).

### 13. 4-{[4-(4-ethoxy-6-{[3-(trifluoromethyl)phenyl]amino}-1,3,5-triazin-2-yl)piperazin-1-yl]carbonyl}benzonitrile

LCMS: M+1=497.4; 1H NMR, DMSO-d6 δ, ppm: 1.35 t (3H); 3.30-4.00 m (8H); 4.35 m (2H); 7.24 d (1H); 7.46 t (1H); 7.64 d (2H); 7.84 d (1H); 7.90 d (2H); 8.30 m (1H); 9.85 s (1H).

### 14. cyclopentyl[4-(4-ethoxy-6-{[3-(trifluoromethyl)phenyl]amino}-1,3,5-triazin-2-yl)piperazin-1-yl]methanone

LCMS: M+1=464.4; 1H NMR, DMSO-d6 δ, ppm: 1.35 t (3H); 1.50-1.90 m (8H); 3.05 m (1H); 3.60 m (4H); 3.85 m (4H); 4.40 m (2H); 7.30 d (1H); 7.50 t (1H); 7.88 d (1H); 8.26 s (1H); 9.50 s (1H).

### 15. cyclopropyl[4-(4-ethoxy-6-{[3-(trifluoromethyl)phenyl]amino}-1,3,5-triazin-2-yl)piperazin-1-yl]methanone

LCMS: M+1=436.4; NMR 1H, DMSO-d6 δ, ppm: 0.80 m (4H); 1.35 t (3H); 2.00 m (1H); 3.50-4.00 m (8H); 4.35 m (2H); 7.24 d (1H); 7.46 t (1H); 7.84 d (1H); 7.84 d (1H); 8.30 s (1H); 9.85 s (1H).

### 16. (3,4-dimethoxyphenyl)[4-(4-ethoxy-6-{[3-(trifluoromethyl)phenyl]amino}-1,3,5-triazin-2-yl)piperazin-1-yl]methanone

LCMS: M+1=532.5; 1H NMR, DMSO-d6 δ, ppm: 1.35 t (3H); 3.65 m (4H); 3.85 m (10H); 4.40 m (2H); 7.04 d (3H); 7.30 d (1H); 7.50 t (1H); 7.88 d (1H); 8.24 s (1H); 9.50 s (1H).

### 17. 2-(3,4-dimethoxyphenyl)-1-[4-(4-ethoxy-6-{[3-(trifluoromethyl)phenyl]amino}-1,3,5-triazin-2-yl)piperazin-1-yl]ethanone

LCMS: M+1=424.4; 1H NMR, DMSO-d6 δ, ppm: 1.35 t (3H); 3.65 m (4H); 3.85 m (12H); 4.40 m (2H); 6.80 d (1H); 6.88 m (2H); 7.30 d (1H); 7.50 t (1H); 7.86 d (1H); 8.24 s (1H); 9.50 s (1H).

### 18. 1-[4-(4-ethoxy-6-{[3-(trifluoromethyl)phenyl]amino}-1,3,5-triazin-2-yl)piperazin-1-yl]-2-(4-fluorophenyl)ethanone

LCMS: M+1=504.4; 1H NMR, DMSO-d6 δ, ppm: 1.35 t (3H); 3.50-3.90 m (10H); 4.35 m (2H); 7.07 t (2H); 7.26 m (3H); 7.46 t (1H); 7.82 d (1H); 8.28 s (1H); 9.85 s (1H).

### 19. cyclohexyl[4-(4-ethoxy-6-{[3-(trifluoromethyl)phenyl]amino}-1,3,5-triazin-2-yl)piperazin-1-yl]methanone

LCMS: M+1=478.5; 1H NMR, DMSO-d6 δ, ppm: 1.20-1.50 m (9H); 1.60-1.80 m (6H); 2.65 m (1H); 3.60 m (4H); 3.80 m (4H); 4.40 m (2H); 7.30 d (1H); 7.50 t (1H); 7.86 d (1H); 8.24 s (1H); 9.50 s (1H).

### 20. 1-[4-(4-ethoxy-6-{[3-(trifluoromethyl)phenyl]amino}-1,3,5-triazin-2-yl)piperazin-1-yl]-2-(phenylamino)ethanone

LCMS: M+1=501.5; 1H NMR, DMSO-d6 δ, ppm: 1.35 m (3H); 3.50 m (4H); 3.80 m (4H); 4.35 m (2H); 7.32 d (1H); 7.40-7.50 m (1H); 7.52 t (1H); 7.84 d (1H); 8.10 s (1H); 8.30 (1H); 9.90 s (1H).

### 21. (3,4-difluorophenyl)[4-(4-ethoxy-6-{[3-(trifluoromethyl)phenyl]amino}-1,3,5-triazin-2-yl)piperazin-1-yl]methanone

LCMS: M+1=508.4; 1H NMR, DMSO-d6 δ, ppm: 1.30 m (3H); 3.60 m (4H); 3.90 m (4H); 4.35 m (2H); 7.30 m (2H); 7.40-7.58 m (3H); 7.88 d (1H); 8.22 s (1H); 9.50 s (1H).

### 22. 3-(3,5-dimethyl-1H-pyrazol-1-yl)-1-[4-(4-ethoxy-6-{[3-(trifluoromethyl)phenyl]amino}-1,3,5-triazin-2-yl)piperazin-1-yl]propan-1-one

LCMS: M+1=518.5; 1H NMR, DMSO-d6 δ, ppm: 1.30 m (3H); 2.10 s (1H); 2.25 s (1H); 3.55 m (4H); 3.75 m (4H); 4.15 m (2H); 4.40 m (2H); 7.30 d (1H); 7.50 t (1H); 7.88 d (1H); 8.24 s (1H); 9.50 s (1H).

### 23. (3-chloro-4-fluorophenyl)[4-(4-ethoxy-6-{[3-(trifluoromethyl)phenyl]amino}-1,3,5-triazin-2-yl)piperazin-1-yl]methanone

LCMS: M+1=524.9; 1H NMR, DMSO-d6 δ, ppm: 1.35 t (3H); 3.30-4.00 m (8H); 4.35 m (2H); 7.24 d (111); 7.38-7.52 m (3H); 7.66 d (1H); 7.84 d (1H); 8.28 s (1H); 9.85 s (1H).

### 24. [4-(4-ethoxy-6-{[3-(trifluoromethyl)phenyl]amino}-1,3,5-triazin-2-yl)piperazin-1-yl](1-ethyl-1H-pyrazol-3-yl)methanone

LCMS: M+1=490.4; 1H NMR, DMSO-d6 δ, ppm: 1.35 t (3H); 1.45 t (3H); 3.60-4.00 m (6H); 4.05-4.30 m (4H); 4.35 m (2H); 6.55 s (1H); 7.24 d (1H); 7.48 t (1H); 7.72-7.88 m (2H); 8.22 s (1H); 9.85 s (1H).

### 25. 4-{4-[(3-chlorophenyl)sulfonyl]piperazin-1-yl}-6-ethoxy-N-[3-(trifluoromethyl)phenyl]-1,3,5-triazin-2-amine

LCMS: M+1=542.9; 1H NMR, DMSO-d6 δ, ppm: 1.35 t (3H); 3.05 m (4H); 3.90 m (4H); 4.35 m (2H); 7.24 d (1H); 7.46 t (1H); 7.60-7.76 m (4H); 7.80 d (1H); 8.20 s (1H); 9.80 (1H).

### 26. 4-ethoxy-6-[4-(5,6,7,8-tetrahydronaphthalen-2-ylsulfonyl)piperazin-1-yl]-N-[3-(trifluoromethyl)phenyl]-1,3,5-triazin-2-amine

LCMS: M+1=562.6; 1H NMR, DMSO-d6 δ, ppm: 1.30 t (3H); 1.80 m (4H); 2.75-3.00 m (8H); 3.90 m (4H); 4.35 m (2H); 7.15-7.50 m (5H); 7.70 d (1H); 8.20 s (1H); 9.85 s (1H).

### Generic Synthesis of Intermediates and Final Compounds for Table 31

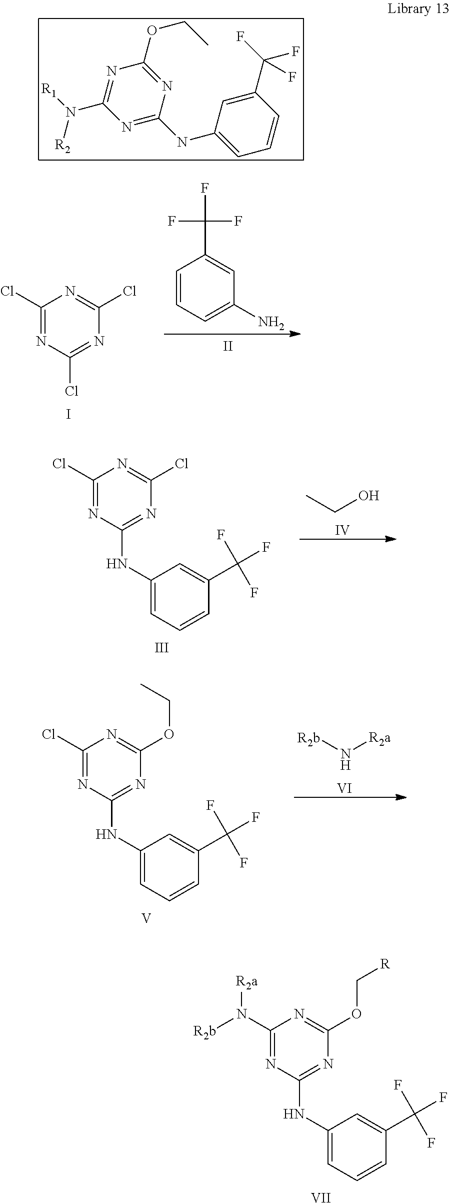

Preparation of 2,4-dichloro-6-(N-3-trifluoromethylanilino)-1,3,5-triazine III. To a stirred solution of 2,4,6-trichlorotriazine I (5 g.) and anhydrous sodium acetate (2.46 g) in dry dioxane (200 ml) was added a solution of aniline II (4.39 g) in 50 mL of dioxane dropwise while the reaction temperature was maintained under 15°

C. After 1 hour of stirring at r.t, the solvent was removed under the reduced pressure and the solid residue was treated successively with saturated aqueous K₂CO₃ solution and then with benzene (2*20 ml). The white solid obtained was dissolved in 100 ml of chloroform; insoluble material was filtered off. The filtrate was dried over sodium sulfate and concentrated under reduced pressure. 2,4-Dichloro-6-(N-3-trifluoro-methylanilino)-1,3,5-triazine 2 was obtained as a white solid in 30% yield (2.56 g).

Preparation of 4-chloro-6-ethoxy-N-[3-(trifluoromethyl)phenyl]-1,3,5-triazine-2-amine V. Sodium (0.075 g, 3.24 mmol) was carefully dissolved in dry ethanol (20 ml) at r.t. 2,4-Dichloro-6-(N-3-trifluoromethyl-anilino)-1,3,5-triazine III (1 g, 3.24 mmol) was dissolved separately in dry ethanol (30 mL), cooled to −30° C., and treated with the sodium ethoxide solution. The reaction mixture was stirred for 3 hours at r.t. LCMS analysis of the reaction mixture demonstrated no starting 2,4-dichloro-6-(N-3-trifluoromethyl-anilino)-1,3,5-triazine. The main product of the reaction was 2-ethoxy-4-chloro-6-(N-3-trifluoromethyl-anilino)-1,3,5-triazine V (M+1=319.4), 90%. Minor components were 2,4-diethoxy-6-(N-3-trifluoromethylanilino)-1,3,5-triazine and 2,4-dioxy-6-(N-3-trifluoromethyl-anilino)-1,3,5-triazine (M+1=329.0 and M+1=273.0, correspondingly), 10% of total. The reaction mixture was concentrated and the crude product V was re-crystallized from hexane. Yield was 60% (0.62 g).

Preparation of 6-ethoxy-N-[3-(trifluoromethyl)phenyl]-1,3,5-triazine-2,4-diamines VII. 4-chloro-6-ethoxy-N-[3-(trifluoromethyl)phenyl]-1,3,5-triazin-2-amine V (0.040 g, 0.125 mmol) was suspended in dioxane (5 ml) in the presence of K₂CO₃ (0.035 g, 0.251 mmol). This mixture was treated with appropriate amines (0.125 mmol) at rt. The reaction mixture was then stirred for 30-40 min at 70-80° C. The reaction mixture was cooled, poured into water and extracted with CH₂Cl₂ or alternatively, CHCl₃. The organic extract was separated, dried over MgSO₄, filtered and concentrated. The residue was filtered, washed with hot hexanes, then with water and dried in vacuo (method a). Noncrystalline products were purified by silica gel chromatography with appropriate eluents (hexanes-ethyl acetate or methylene chloride-ethanol) (method b).

TABLE 31

| En | Structure | IUPAC Name | MW | Formula |
|---|---|---|---|---|
| 1 | 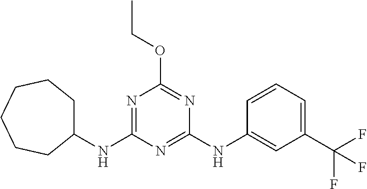 | N-cycloheptyl-6-ethoxy-N′-[3- (trifluoromethyl)phenyl]-1,3,5- triazine-2,4-diamine | 395.4 | C19H24F3N5O |
| 2 | 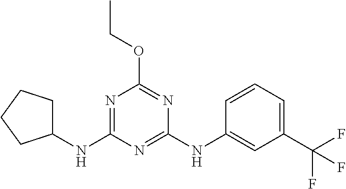 | N-cyclopentyl-6-ethoxy-N′-[3- (trifluoromethyl)phenyl]-1,3,5- triazine-2,4-diamine | 367.4 | C17H20F3N5O |
| 3 | 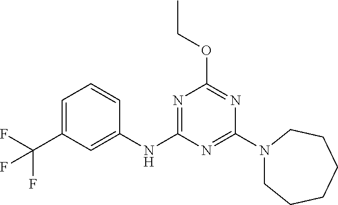 | 4-(azepan-1-yl)-6-ethoxy-N-[3- (trifluoromethyl)phenyl]-1,3,5- triazin-2-amine | 381.4 | C18H22F3N5O |
| 4 | 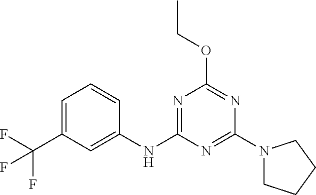 | 4-ethoxy-6-(pyrrolidin-1-yl)-N-[3- (trifluoromethyl)phenyl]-1,3,5- triazin-2-amine | 353.3 | C16H18F3N5O |
| 5 | 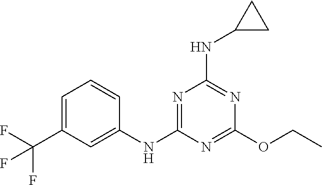 | N-cyclopropyl-6-ethoxy-N′-[3- (trifluoromethyl)phenyl]-1,3,5- triazine-2,4-diamine | 339.3 | C15H16F3N5O |
| 6 | 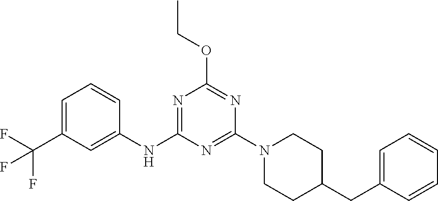 | 4-(4-benzylpiperidin-1-yl)-6- ethoxy-N-[3- (trifluoromethyl)phenyl]-1,3,5- triazin-2-amine | 457.5 | C24H26F3N5O |
| 7 | 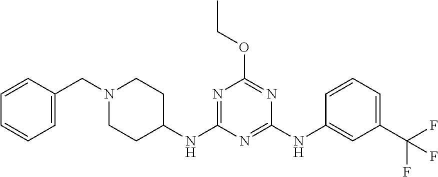 | N-(1-benzylpiperidin-4-yl)-6- ethoxy-N′-[3- (trifluoromethyl)phenyl]-1,3,5- triazine-2,4-diamine | 472.5 | C24H27F3N6O |
| 8 | 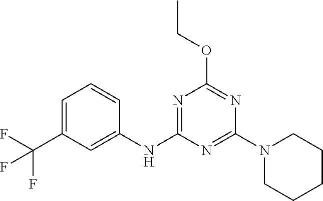 | 4-ethoxy-6-(piperidin-1-yl)-N-[3- (trifluoromethyl)phenyl]-1,3,5- triazin-2-amine | 367.4 | C17H20F3N5O |
| 9 | 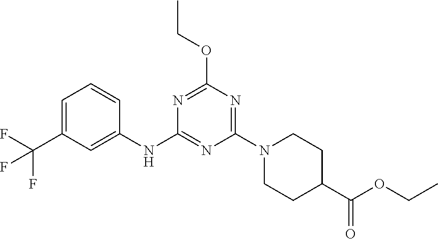 | ethyl 1-(4-ethoxy-6-{[3- (trifluoromethyl)phenyl]amino}- 1,3,5-triazin-2-yl)piperidine-4- carboxylate | 439.4 | C20H24F3N5O3 |

### Procedures and Analytical Data for Table 31

Entries 1 to 9 are from Library 31.

### 1. N-cycloheptyl-6-ethoxy-N′-[3-(trifluoromethyl)phenyl]-1,3,5-triazine-2,4-diamine

LCMS: M+1=395.4; 1H NMR, DMSO-d6 δ, ppm: 1.3 t (3H); 1.40-1.80 m (10H); 1.95 m (2H); 4.05 m (1H); 4.35 m (2H); 6.92 d (1H); 7.28 d (1H); 7.50 t (1H); 7.98 d (1H); 8.24 s (1H); 9.30 s (1H).

### 2. N-cyclopentyl-6-ethoxy-N′-[3-(trifluoromethyl)phenyl]-1,3,5-triazine-2,4-diamine

LCMS: M+=368.5 1H NMR, DMSO-d6 δ, ppm: 1.35 m (3H); 1.50-2.10 m (8H); 4.20-4.45 m (3H); 6.80 s (1H); 7.20 d (1H); 7.45 t (1H); 7.95 d (1H); 8.30 s (1H); 8.50 s (1H)

### 3. 4-(azepan-1-yl)-6-ethoxy-N-[3-(trifluoromethyl)phenyl]-1,3,5-triazin-2-amine

LCMS: M+1=381.4; NMR CDCl3, ppm: 1.40 t (3H); 1.80 m (4H); 3.55 m (4H); 4.45 m (2H); 7.20-7.55 m (5H); 8.45 s (1H).

### 4. 4-ethoxy-6-(pyrrolidin-1-yl)-N-[3-(trifluoromethyl)phenyl]-1,3,5-triazin-2-amine

LCMS: M+=354.5 NMR CDCl3, ppm: 1.45 t (3H); 2.00 m (4H); 3.60 m (4H); 4.45 m (2H); 7.15-7.35 m (3H); 7.35-7.60 m (2H); 8.40 s (1H)

### 5. N-cyclopropyl-6-ethoxy-N′-[3-(trifluoromethyl)phenyl]-1,3,5-triazine-2,4-diamine

LCMS: M+1=339.3; 1H NMR, DMSO-d6 δ, ppm: 0.55 m (2H); 0.75 m (2H); 1.35 t (3H); 2.85 m (1H); 4.40 m (2H); 7.30 m (2H); 7.50 t (1H); 8.0 d (1H); 8.35 s (1H); 9.40 s 1H).

### 6. 4-(4-benzylpiperidin-1-yl)-6-ethoxy-N-[3-(trifluoromethyl)phenyl]-1,3,5-triazin-2-amine

LCMS: M+=458.6; 1H NMR CDCl3, δ, ppm: 1.10-1.50 m (5H); 1.55-1.95 m (4H); 2.50-3.00 m (4H); 4.40 m (2H); 4.75 m (2H); 7.05-7.60 m (10H); 8.25 s (1H)

### 7. N-(1-benzylpiperidin-4-yl)-6-ethoxy-N′-[3-(trifluoromethyl)phenyl]-1,3,5-triazine-2,4-diamine

LCMS: M+=473.4 1H NMR, DMSO-d6 δ, ppm: 1.30 m (3H); 1.60 m (2H); 1.85 m (2H); 2.10 t (2H); 2.75-3.10 m (7H); 3.50 s (2H); 3.85 m (1H); 4.35 m (2H); 6.95 s (1H); 7.10-7.30 m (7H); 7.95 s (1H); 8.25 s (1H); 9.30 s (1H)

### 8. 4-ethoxy-6-(piperidin-1-yl)-N-[3-(trifluoromethyl)phenyl]-1,3,5-triazin-2-amine

LCMS: M+=368.5 1H NMR CDCl3, ppm: 1.40 m (3H); 1.50-1.75 m (7H); 3.80 m (4H); 4.40 m (2H); 7.15-7.35 m (3H); 7.40-7.60 m (2H); 8.30 s (1H)

### 9. ethyl 1-(4-ethoxy-6-{[3-(trifluoromethyl)phenyl]amino}-1,3,5-triazin-2-yl)piperidine-4-carboxylate

LCMS: M+1=439.4; 1H NMR (DMSO-d6, 90° C., ppm): =1.22 t (3H), 1.41 t (3H), 1.6 m (2H), 1.96 m (2H), 2.75 m (1H), 3.21 m (1H), 4.12 m (2H), 4.42 m (2H), 7.51 m (1H), 7.58 dd (2H), 8.31 s (1H), 9.42 s (1H).

### Generic Synthesis of Intermediates and Final Compounds for Table 32

There are several approaches to make the table of compounds. One approach is R4-R6-R2 route. In this route, the R6 fragments were either introduced as compound 4 or by removing BOC-group in 2 following modification of the R6 fragments to get desired compounds via various reductive aminations, acylation, sulfonation, alkylation, and arylations, and etc. The R4-R6-R2 route and procedures to prepare final compounds are following.

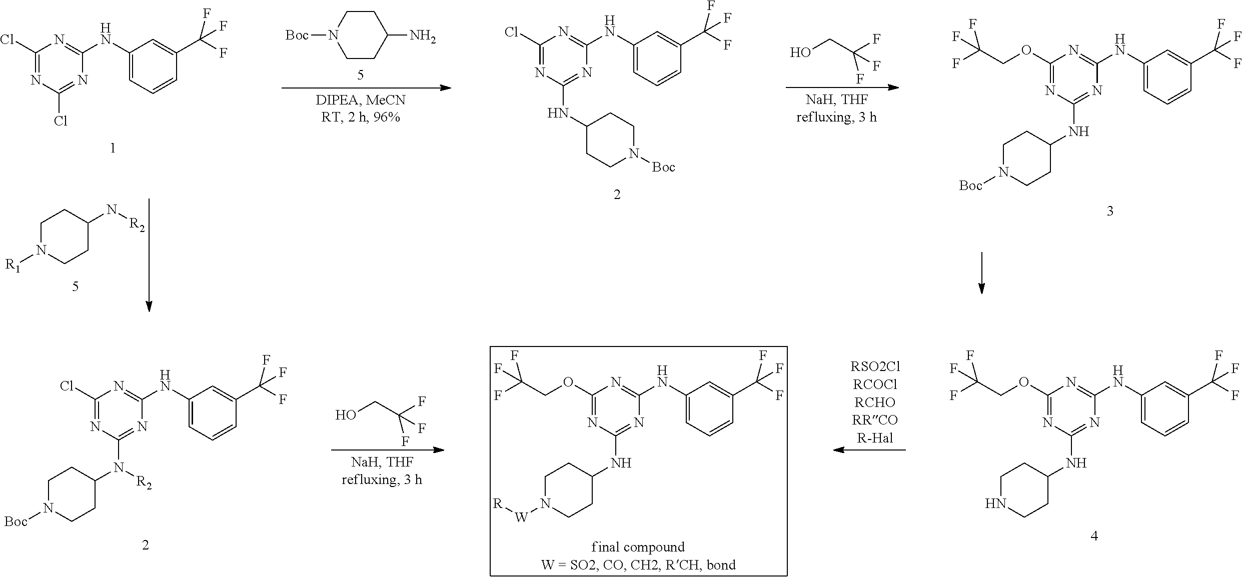

### 4-[4-Chloro-6-(3-trifluoromethyl-phenylamino)-[1,3,5]triazin-2-ylamino]-piperidine-1-carboxylic acid tert-butyl ester (2)

A mixture of compound 1 (7.00 g, 22.7 mmol), 4-amino-piperidine-1-carboxylic acid tert-butyl ester (4.53 g, 22.7 mmol), DIPEA (3.23 g, 25.0 mmol) and acetonitrile (100 mL) was stirred at room temperature for 2 hours, then poured into water (200 mL) and stirred for 30 minutes. The formed precipitate was collected by filtration, washed with hexane and dried giving compound 2. Yield 10.25 g, 96%. MW 472.90. MS (APCI+), m/z 473, 475 [M+H]⁺ (APCI−), m/z 471, 473 [M−H]⁻

### 4-[4-(2,2,2-Trifluoro-ethoxy)-6-(3-trifluoromethyl-phenylamino)-[1,3,5]triazin-2-ylamino]-piperidine-1-carboxylic acid tert-butyl ester (3)

Sodium hydride (60% in oil, 1.69 g, 42.3 mmol) was added portionwise to a solution of 2,2,2-trifluoroethanol (6 mL) in THF (50 mL) at room temperature. The resulting mixture was stirred at room temperature for 15 minutes. Then compound 14 (10.00 g, 21.1 mmol) was added at room temperature. The obtained mixture was stirred at refluxing for 3 hours, cooled down to room temperature, diluted with water and extracted with dichloromethane (2×100 mL). The combined organic phases were dried over potassium carbonate and concentrated. The residue was triturated with hexane and dried giving compound 3. Yield 10.84 g, 96%. MW 536.48. MS (APCI+), m/z 537 [M+H]⁺

### N-Piperidin-4-yl-6-(2,2,2-trifluoro-ethoxy)-N′-(3-trifluoromethyl-phenyl)-[1,3,5]triazine-2,4-diamine (4)

A solution of compound 3 (10.84 g, 20.22 mmol) in dioxane saturated with HCl (16%, 50 mL) was stirred at room temperature for 2 hours (TLC control). Then the mixture was poured in 5% aqueous solution of sodium hydroxide (200 mL). The formed precipitate was collected by filtration, washed with water and dried giving compound 4. Yield 8.60 g, 98%. S (APCI+), m/z 437 [M+H]⁺.

### Generic Procedure for Synthesis of Final Compounds:

Method A: A mixture of the compound 4 (0.55 mmol), sulfonyl chloride (0.82 mmol), NEt₃ (138 mg, 1.36 mmol), acetonitrile (5 mL) was stirred at room temperature for 8 hours and diluted with water. The formed precipitate was filtered off, purified by recrystallization or/and column chromatography on silica gel giving a final compound.

Method B: A mixture of compound 4 (1.03 mmol), corresponding sulfonyl chloride (1.24 mmol) and K₂CO₃ (3.09 mmol) in dichloromethane (10 mL) was stirred at room temperature for 8 hours, diluted with water and extracted with dichloromethane. The combined organic phases were dried over Na₂SO₄ and concentrated. Purification by column chromatography on silica gel (ethyl acetate/hexane) gave a final compound.

TABLE 32

| En | Structure | IUPAC name | MW | Formula |
|---|---|---|---|---|
| 1 | 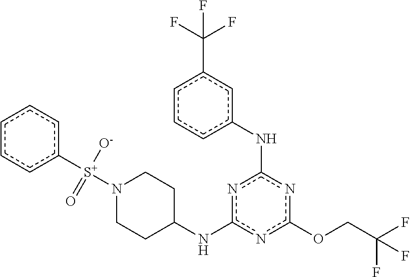 | N-[1-(phenylsulfonyl)piperidin-4- yl]-6-(2,2,2-trifluoroethoxy)-N′-[3- (trifluoromethyl)phenyl]-1,3,5- triazine-2,4-diamine | 576.5 | C23H22F6N6O3S |
| 2 | 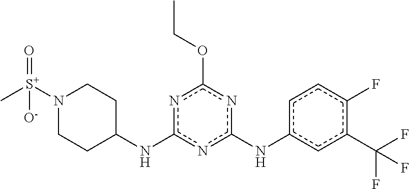 | 6-ethoxy-N-[4-fluoro-3- (trifluoromethyl)-phenyl]-N′-[1- (methylsulfonyl)piperidin-4-yl)- 1,3,5-triazine-2,4-diamine | 478.5 | C18H22F4N6O3S |
| 3 | 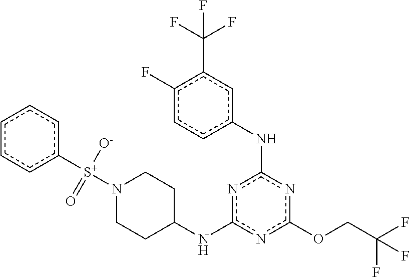 | N-[4-fluoro-3- (trifluoromethyl)phenyl]-N′-[1- (phenylsulfonyl)piperidin-4-yl]-6- (2,2,2-trifluoroethoxy)-1,3,5- triazine-2,4-diamine | 594.5 | C23H21F7N6O3S |
| 4 | 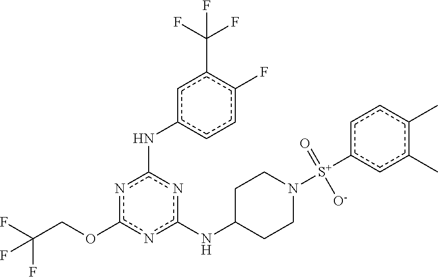 | N-{1-[(3,4- dimethylphenyl)sulfonyl]piperidin- 4-yl}-N′-[4-fluoro-3- (trifluoromethyl)phenyl]-6-(2,2,2- trifluoroethoxy)-1,3,5-triazine- 2,4-diamine | 622.6 | C25H25F7N6O3S |
| 5 | 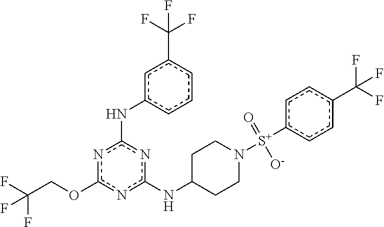 | 6-(2,2,2-trifluoroethoxy)-N-[3- (trifluoromethyl)-phenyl]-N′-(1- {[4-(trifluoromethyl)phenyl]sul- fonyl}piperidin-4-yl)-1,3,5- triazine-2,4-diamine | 644.5 | C24H21F9N6O3S |
| 6 | 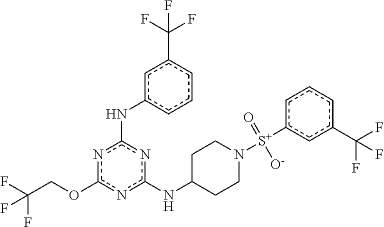 | 6-(2,2,2-trifluoroethoxy)-N-[3- (trifluoromethyl)-phenyl]-N′-(1- {[3-(trifluoromethyl)phenyl]sul- fonyl}piperidin-4-yl)-1,3,5- triazine-2,4-diamine | 644.5 | C24H21F9N6O3S |
| 7 | 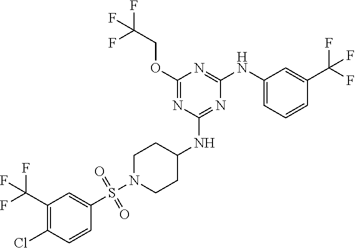 | N*2*-[1-(2-Chloro-5- trifluoromethyl-benzenesulfonyl)- piperidin-4-yl]-6-(2,2,2-trifluoro- ethoxy)-N*4*-(3-trifluoromethyl- phenyl)-1,3,5-triazine-2,4- diamine | 678.1 | C24H20ClF9N6O3S |
| 8 | 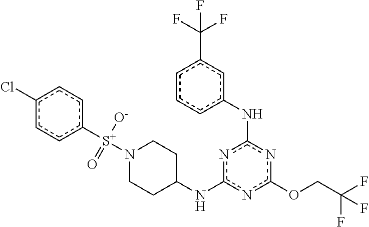 | N-{1-[(4- chlorophenyl)sulfonyl]piperidin- 4-yl}-6-(2,2,2-trifluoroethoxy)-N′- [3-(trifluoro-methyl)phenyl]-1,3,5- triazine-2,4-diamine | 611.0 | C23H21ClF6N6O3S |
| 9 | 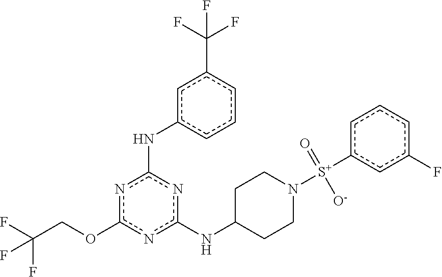 | N-{1-[(3- fluorophenyl)sulfonyl]piperidin-4- yl}-6-(2,2,2-trifluoroethoxy)-N′-[3- (trifluoro-methyl)phenyl]-1,3,5- triazine-2,4-diamine | 594.5 | C23H21F7N6O3S |
| 10 | 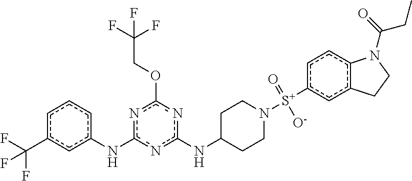 | 1-{5-[(4-{[4-(2,2,2- trifluoroethoxy)-6-{[3- (trifluoromethyl)phenyl]amino}- 1,3,5-triazin-2-yl]amino}piperidin- 1-yl)sulfonyl]-2,3-dihydro-1H- indol-1-yl}propan-1-one | 673.6 | C28H29F6N7O4S |
| 11 | 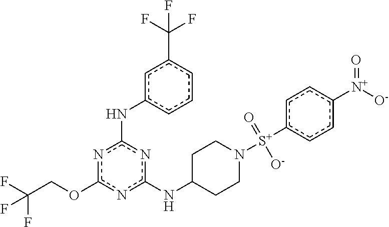 | N-{1-[(4- nitrophenyl)sulfonyl]piperidin-4- yl}-6-(2,2,2-trifluoroethoxy)-N′-[3- (trifluoro-methyl)phenyl]-1,3,5- triazine-2,4-diamine | 621.5 | C23H21F6N7O5S |
| 12 | 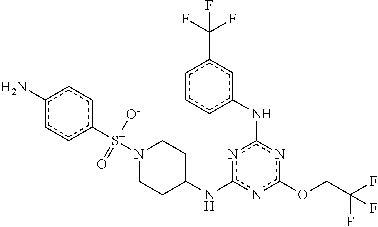 | N-{1-[(4- aminophenyl)sulfonyl]piperidin- 4-yl}-6-(2,2,2-trifluoroethoxy)-N′- [3-(trifluoro-methyl)phenyl]-1,3,5- triazine-2,4-diamine | 591.5 | C23H23F6N7O3S |
| 13 | 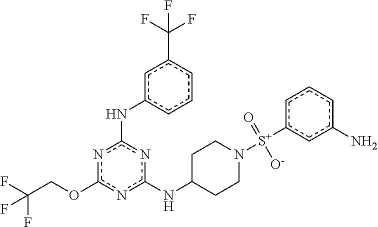 | N-{1-[(3- aminophenyl)sulfonyl]piperidin- 4-yl}-6-(2,2,2-trifluoroethoxy)-N′- [3-(trifluoro-methyl)phenyl]-1,3,5- triazine-2,4-diamine | 591.5 | C23H23F6N7O3S |
| 14 | 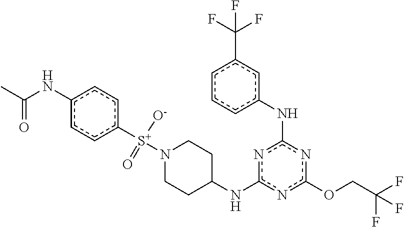 | N-{4-[(4-{[4-(2,2,2- trifluoroethoxy)-6-{[3- (trifluoromethyl)phenyl]amino}- 1,3,5-triazin-2-yl]amino}piperidin- 1-yl)sulfonyl]phenyl}acetamide | 633.6 | C25H25F6N7O4S |
| 15 | 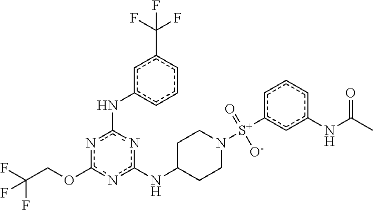 | N-{3-[(4-{[4-(2,2,2- trifluoroethoxy)-6-{[3- (trifluoromethyl)phenyl]amino}- 1,3,5-triazin-2-yl]amino}piperidin- 1-yl)sulfonyl]phenyl}acetamide | 633.6 | C25H25F6N7O4S |
| 16 | 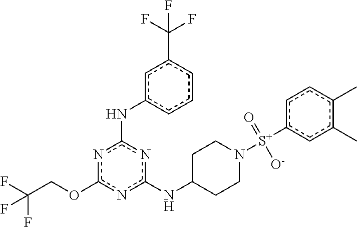 | N-{1-[(3,4- dimethylphenyl)sulfonyl]piperidin- 4-yl}-6-(2,2,2-trifluoroethoxy)-N′- [3-(trifluoro-methyl)phenyl]-1,3,5- triazine-2,4-diamine | 604.6 | C25H26F6N6O3S |
| 17 | 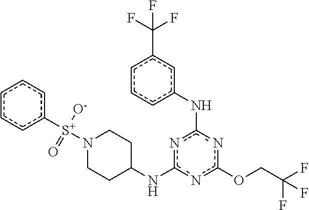 | 4-{[1-(phenylsulfonyl)piperidin-4- yl]oxy}-6-(2,2,2-trifluoroethoxy)- N-[3-(trifluoromethyl)phenyl]- 1,3,5-triazin-2-amine | 577.5 | C23H21F6N5O4S |
| 18 | 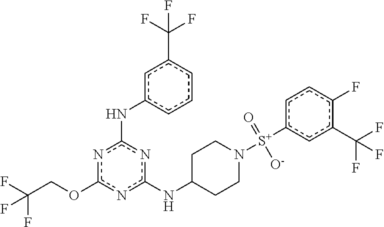 | N-(1-{[4-fluoro-3- (trifluoromethyl)phenyl]- sulfonyl}piperidin-4-yl)-6-(2,2,2- trifluoroethoxy)-N′-[3- (trifluoromethyl)phenyl]-1,3,5- triazine-2,4-diamine | 662.5 | C24H20F10N6O3S |
| 19 | 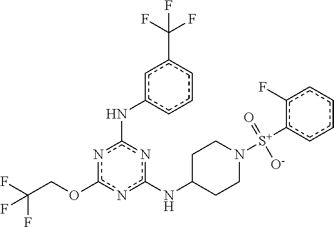 | N-{1-[(2- fluorophenyl)sulfonyl]piperidin-4- yl}-6-(2,2,2-trifluoroethoxy)-N′-[3- (trilfluoro-methyl)phenyl]-1,3,5- triazine-2,4-diamine | 594.5 | C23H21F7N6O3S |
| 20 | 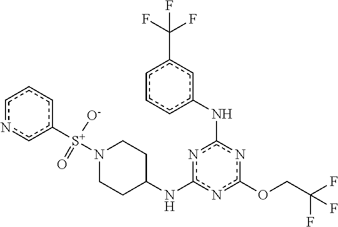 | N-[1-(pyridin-3- ylsulfonyl)piperidin-4-yl]-6-(2,2,2- trifluoroethoxy)-N′-[3-(trifluoro- methyl)phenyl]-1,3,5-triazine- 2,4-diamine | 577.5 | C22H21F6N7O3S |
| 21 | 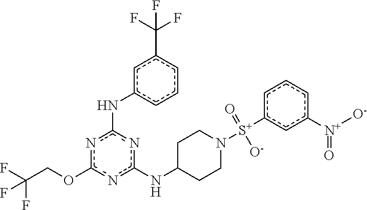 | N-{1-[(3- nitrophenyl)sulfonyl]piperidin-4- yl}-6-(2,2,2-trifluoroethoxy)-N′-[3- (trifluoromethyl)-phenyl]-1,3,5- triazine-2,4-diamine | 621.5 | C23H21F6N7O5S |
| 22 | 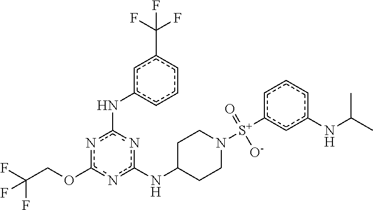 | N-(1-{[3-(propan-2- ylamino)phenyl]-sulfonyl}- piperidin-4-yl)-6-(2,2,2- trifluoroethoxy)-N′-[3- (trifluoromethyl)phenyl]-1,3,5- triazine-2,4-diamine | 633.6 | C26H29F6N7O3S |
| 23 | 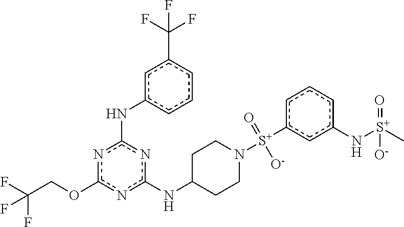 | N-{3-[(4-{[4-(2,2,2- trifluoroethoxy)-6-{[3- (trifluoromethyl)phenyl]amino}- 1,3,5-triazin-2-yl]amino}piperidin- 1- yl)sulfonyl]phenyl} methanesulfonamide | 669.6 | C24H25F6N7O5S2 |
| 24 | 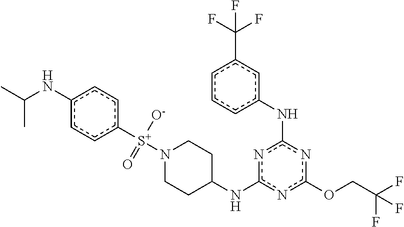 | N-(1-{[4-(propan-2- ylamino)phenyl]sulfonyl}- piperidin-4-yl)-6-(2,2,2- trifluoroethoxy)-N′-[3- (trifluoromethyl)phenyl]-1,3,5- triazine-2,4-diamine | 633.6 | C26H29F6N7O3S |
| 25 | 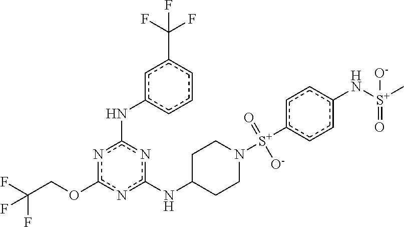 | N-{4-[(4-{[4-(2,2,2- trifluoroethoxy)-6-{[3- (trifluoromethyl)phenyl]amino}- 1,3,5-triazin-2-yl]amino}piperidin- 1- yl)sulfonyl]phenyl} methanesulfonamide | 669.6 | C24H25F6N7O5S2 |
| 26 | 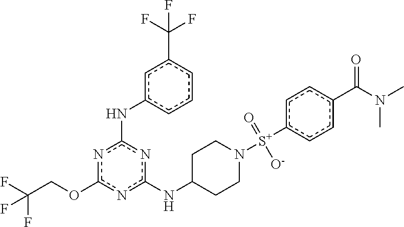 | N,N-dimethyl-4-[(4-{[4-(2,2,2- trifluoroethoxy)-6-{[3- (trifluoromethyl)phenyl]amino}- 1,3,5-triazin-2-yl]amino}piperidin- 1-yl)sulfonyl]benzamide | 647.6 | C26H27F6N7O4S |
| 27 | 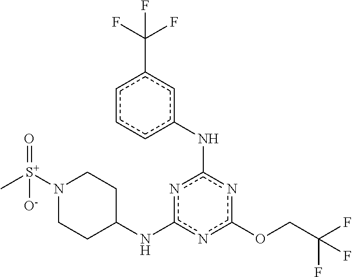 | N-[1-(methylsulfonyl)piperidin-4- yl]-6-(2,2,2-trifluoroethoxy)-N′-[3- (trifluoromethyl)phenyl]-1,3,5- triazine-2,4-diamine | 514.5 | C18H20F6N6O3S |
| 28 | 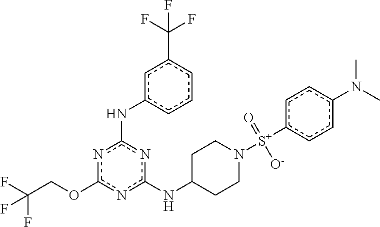 | N-(1-{[4- (dimethylamino)phenyl]sulfon- yl}piperidin-4-yl)-6-(2,2,2- trifluoroethoxy)-N′-[3- (trifluoromethyl)phenyl]-1,3,5- triazine-2,4-diamine | 619.6 | C25H27F6N7O3S |
| 29 | 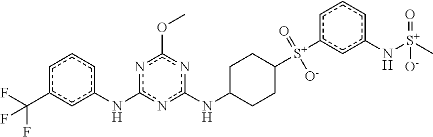 | N-[3-({4-[(4-methoxy-6-{[3- (trifluoromethyl)-phenyl]amino}- 1,3,5-triazin-2-yl)amino] piperidin-1- yl}sulfonyl)phenyl] methanesulfonamide | 601.6 | C23H26F3N7O5S2 |
| 30 | 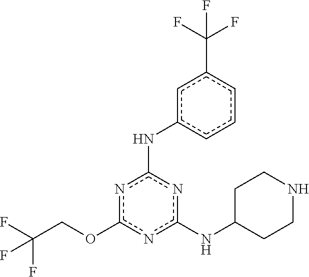 | N-(piperidin-4-yl)-6-(2,2,2- trifluoroethoxy)-N′-[3- (trifluoromethyl)phenyl]-1,3,5- triazine-2,4-diamine | 436.4 | C17H18F6N6O |
| 31 | 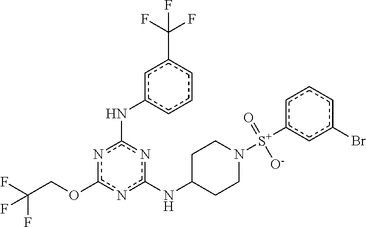 | N-{1-[(3- bromophenyl)sulfonyl]piperidin- 4-yl}-6-(2,2,2-trifluoroethoxy)-N′- [3-(trifluoro-methyl)phenyl]-1,3,5- triazine-2,4-diamine | 655.4 | C23H21BrF6N6O3S |
| 32 | 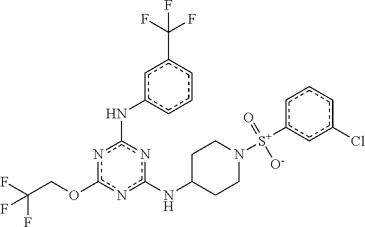 | N-{1-[(3- chlorophenyl)sulfonyl]piperidin- 4-yl}-6-(2,2,2-trifluoroethoxy)-N′- [3-(trifluoro-methyl)phenyl]-1,3,5- triazine-2,4-diamine | 611.0 | C23H21ClF6N6O3S |
| 33 | 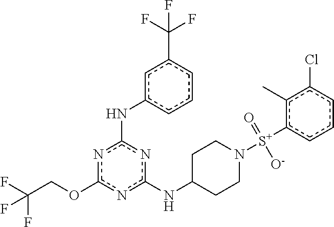 | N-{1-[(3-chloro-2- methylphenyl)sulfonyl]-piperidin- 4-yl}-6-(2,2,2-trifluoroethoxy)-N′- [3-(trifluoromethyl)phenyl]-1,3,5- triazine-2,4-diamine | 625.0 | C24H23ClF6N6O3S |
| 34 | 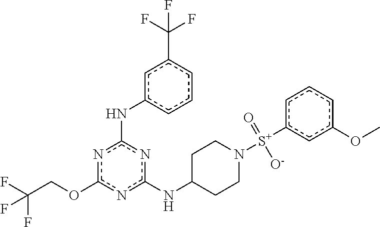 | N-{1-[(3- methoxyphenyl)sulfonyl]piperidin- 4-yl}-6-(2,2,2-trifluoroethoxy)-N′- [3-(trifluoro-methyl)phenyl]-1,3,5- triazine-2,4-diamine | 606.5 | C24H24F6N6O4S |
| 35 | 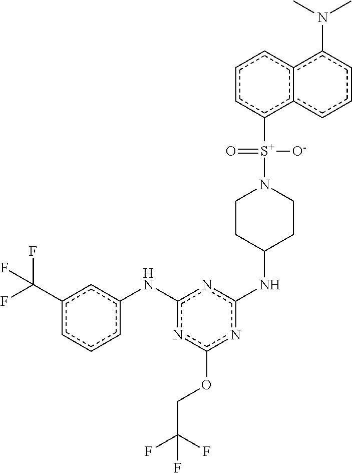 | N-(1-{[5- (dimethylamino)naphthalen-1- yl]sulfonyl}piperidin-4-yl)-6- (2,2,2-trifluoroethoxy)-N′-[3- (trifluoromethyl)phenyl]-1,3,5- triazine-2,4-diamine | 669.6 | C29H29F6N7O3S |
| 36 | 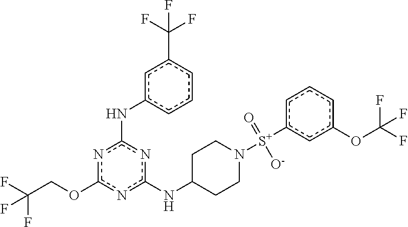 | 6-(2,2,2-trifluoroethoxy)-N-(1-{[3- (trifluoromethoxy)phenyl]sulfonyl} piperidin-4-yl)-N′-[3- (trifluoromethyl)phenyl]-1,3,5- triazine-2,4-diamine | 660.5 | C24H21F9N6O4S |
| 37 | 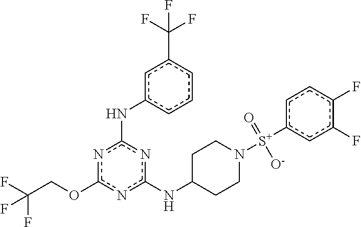 | N-{1-[(3,4- difluorophenyl)sulfonyl]piperidin- 4-yl}-6-(2,2,2-trifluoroethoxy)-N′- [3-(trifluoro-methyl)phenyl]-1,3,5- triazine-2,4-diamine | 612.5 | C23H20F8N6O3S |
| 38 | 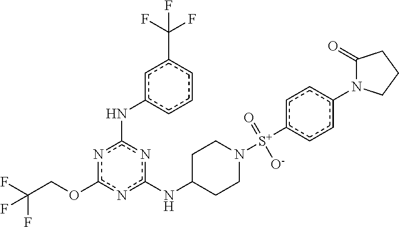 | 1-{4-[(4-{[4-(2,2,2- trifluoroethoxy)-6-{[3- (trifluoromethyl)phenyl]amino}- 1,3,5-triazin-2-yl]amino}piperidin- 1-yl)sulfonyl]phenyl}pyrrolidin-2- one | 659.6 | C27H27F6N7O4S |
| 39 | 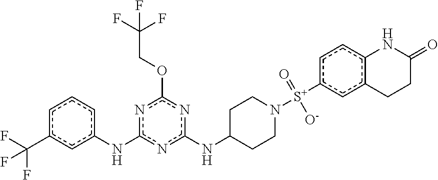 | 6-[(4-{[4-(2,2,2-trifluoroethoxy)-6- {[3- (trifluoromethyl)phenyl]amino}- 1,3,5-triazin-2-yl]amino}piperidin- 1-yl)sulfonyl]-3,4- dihydroquinolin-2(1H)-one | 645.6 | C26H25F6N7O4S |
| 40 | 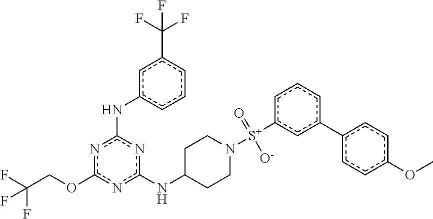 | N-{1-(4′-methoxybiphenyl-3- yl)sulfonyl]-piperidin-4-yl}-6- (2,2,2-trifluoroethoxy)-N′-[3- (trifluoromethyl)phenyl]-1,3,5- triazine-2,4-diamine | 682.6 | C30H28F6N6O4S |
| 41 | 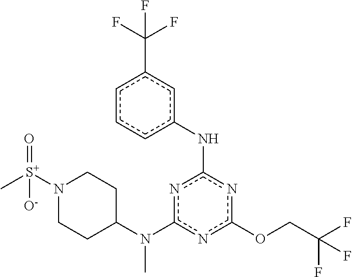 | N-methyl-N-[1- (methylsulfonyl)piperidin-4-yl]-6- (2,2,2-trifluoroethoxy)-N′-[3- (trifluoro-methyl)phenyl]-1,3,5- triazine-2,4-diamine | 528.5 | C19H22F6N6O3S |
| 42 | 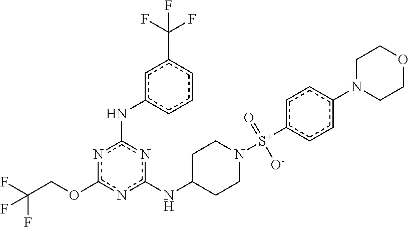 | N-(1-{[4-(morpholin-4- yl)phenyl]sulfonyl}-piperidin-4- yl)-6-(2,2,2-trifluoroethoxy)-N′-[3- (trifluoromethyl)phenyl]-1,3,5- triazine-2,4-diamine | 661.6 | C27H29F6N7O4S |
| 43 | 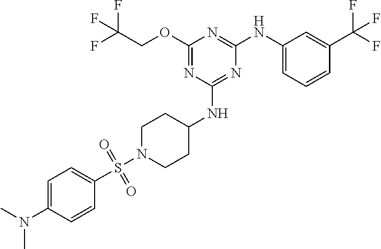 | N-[1-(4-Dimethylamino- benzenesulfonyl)-piperidin-4-yl]- 6-(2,2,2-trifluoro-ethoxy)-N′-(3- trifluoromethyl-phenyl)- [1,3,5]triazine-2,4-diamine | 619.2 | C25H27F6N7O3S |

### Procedures and Analytical Data for Table 32.

### 1. 1. N-(1-Benzenesulfonyl-piperidin-4-yl)-6-(2,2,2-trifluoro-ethoxy)-N′-(3-trifluoromethyl-phenyl)-[1,3,5]triazine-2,4-diamine

Yield 220 mg, 76%. ¹H-NMR (400 MHz, DMSO-D₆) δ_{H}: 1.60 (2H, m), 1.92 (2H, m), 2.38 (2H, m), 3.62 (2H, m), 3.79 (1H, broad), 4.92 (2H, superposition of two q, J=7.5 Hz), 7.27 (1H, superposition of two d, J=8.5 Hz, Z/E forms), 7.45 (1H, superposition of two d, J=8.5 Hz, Z/E forms), 7.65 (3H, broad peak, Z/E forms), 7.75 (3H, broad peak, Z/E forms), 7.82-7.98 (1H, two broad peaks, Z/E forms), 8.08-8.32 (1H, two broad peaks, Z/E forms), 9.70-9.90 (1H, two broad peaks, Z/E forms). MW 576.52. LCMS t_{R} (min): 2.11. MS (APCI+), m/z 577.10 [M+H]⁺. HPLC t_{R} (min): 17.49. M_{P} 195-197° C.

### 2. 6-Ethoxy-N-(4-fluoro-3-trifluoromethyl-phenyl)-N′-(1-methanesulfonyl-piperidin-4-yl)-[1,3,5]triazine-2,4-diamine

Yield 160 mg, 58%. ¹H-NMR (400 MHz, DMSO-D₆) δ_{H}: 1.28 (3H, broad t), 1.59 (2H, m), 1.94 (2H, m), 2.82 (2H, m), 2.91 (3H, s), 3.61 (2H, m), 3.92 (1H, broad peak, Z/E forms), 4.32 (2H, broad q, J=7.5 Hz), 7.38 (1H, broad peak, Z/E forms), 7.38-7.54 (1H, two broad peaks, Z/E forms), 7.91-8.11 (1H, two broad peaks, Z/E forms), 8.11-8.31 (1H, two broad peaks, Z/E forms), 9.51-9.71 (1H, two broad peaks, Z/E forms). MW 478.95. LCMS t_{R} (min): 1.88. MS (APCI+), m/z 479.08, 461.01 [M+H]⁺. HPLC t_{R} (min): 14.53. M_{P} 204-206° C.

### 3. N-(1-Benzenesulfonyl-piperidin-4-yl)-N′-(4-fluoro-3-trifluoromethyl-phenyl)-6-(2,2,2-trifluoro-ethoxy)-[1,3,5]triazine-2,4-diamine

¹H-NMR (400 MHz, DMSO-D₆) δ_{H}: 1.61 (2H, m), 1.91 (2H, m), 2.39 (2H, m), 3.61 (2H, m), 3.71 (1H, broad peak, Z/E forms), 4.91 (2H, superposition of two q, J=7.5 Hz), 7.38 (1H, superposition of two m), 7.61 (2H, broad peak, Z/E forms), 7.71 (4H, broad peak, Z/E forms), 7.79-7.99 (1H, two broad peaks, Z/E forms), 8.19-8.31 (1H, two broad peaks, Z/E forms), 9.62-9.92 (1H, two broad peaks, Z/E forms). MW 594.51. LCMS t_{R} (min): 2.17. MS (APCI+), m/z 595.18 [M+H]⁺. HPLC t_{R} (min): 17.76. M_{P} 209-212° C.

### 4. N-[1-(3,4-Dimethyl-benzenesulfonyl)-piperidin-4-yl]-N′-(4-fluoro-3-trifluoro-methyl-phenyl)-6-(2,2,2-trifluoro-ethoxy)-[1,3,5]triazine-2,4-diamine

¹H-NMR (400 MHz, DMSO-D₆) δ_{H}: 1.61 (2H, m), 1.91 (2H, m), 2.32 (6H, s), 2.41 (2H, m), 3.21 (2H, m), 3.61 (2H, m), 3.69 (1H, broad peak, Z/E forms), 7.29 (2H, broad peak, Z/E forms), 7.35-7.38 (1H, two broad peaks, Z/E forms), 7.49 (1H, broad peak, Z/E forms), 7.58-7.65 (1H, two broad peaks, Z/E forms), 7.81-7.95 (1H, two broad peaks, Z/E forms), 8.09-8.31 (1H, two broad peaks, Z/E forms), 9.65-9.81 (1H, two broad peaks, Z/E forms). MW 622.56. LCMS t_{R} (min): 2.26. MS (APCI−), m/z 620.94 [M−H]⁻. HPLC t_{R} (min): 18.66. M_{P} 248-249° C.

### 5. 6-(2,2,2-Trifluoro-ethoxy)-N*2*-[1-(4-trifluoromethyl-benzenesulfonyl)-piperidin-4-yl]-N*4*-(3-trifluoromethyl-phenyl)-1,3,5-triazine-2,4-diamine

Yield 177 mg, 60%. ¹H-NMR (400 MHz, DMSO-D₆) δ_{H}: 1.60 (2H, m), 1.94 (2H, m), 2.40 (2H, m), 3.70 (2H, m), 3.80 (1H, broad peak), 4.92 (2H, superposition of two q, J=7.5 Hz, Z/E forms), 7.25-7.30 (1H, superposition of two d, J=8.5 Hz, Z/E forms), 7.42-7.50 (1H, superposition of two t, J=8.5 Hz, Z/E forms), 7.63 (1H, broad, Z/E forms), 7.81-7.93 (1H, two broad peaks, Z/E forms), 7.97 (2H, broad peak, Z/E forms), 8.08-8.13 (2H, two d, J=8.5 Hz, Z/E forms), 8.34 (1H, broad, Z/E forms), 9.70-9.90 (1H, two broad peaks, Z/E forms). MW 644.5. LCMS t_{R} (min): 2.19. MS (APCI+), m/z 645.07 [M+H]⁺. HPLC t_{R} (min): 18.57. M_{P} 223-225° C.

### 6. 6-(2,2,2-Trifluoro-ethoxy)-N-[1-(3-trifluoromethyl-benzenesulfonyl)-piperidin-4-yl]-N′-(3-trifluoromethyl-phenyl)-[1,3,5]triazine-2,4-diamine

Yield 256 mg, 86%. ¹H-NMR (400 MHz, DMSO-D₆) δ_{H}: 1.60 (2H, m), 1.92 (2H, m), 2.40 (2H, m), 3.69 (2H, m), 3.80 (1H, broad peaks, Z/E forms), 4.90 (2H, superposition of two q, Z/E forms), 7.23-7.29 (1H, two d, J=8.5 Hz, Z/E forms), 7.40-7.51 (1H, two t, J=8.5 Hz, Z/E forms), 7.62 (1H, broad peak, Z/E forms), 7.82-7.91 (1H, two broad peaks, Z/E forms), 7.96 (2H, m), 8.07 (1H, d, J=8.5 Hz), 8.12 (1H, broad d, J=8.5 Hz), 8.33 (1H, broad peaks, Z/E forms), 9.60-9.80 (1H, two broad peaks, Z/E forms). MW 644.52. LCMS t_{R} (min): 2.18. MS (APCI+), m/z 644.88, 645.91 [M+H]⁺. HPLC t_{R} (min): 18.72. M_{P} 223-224° C.

### 7. N*2*-[1-(2-Chloro-5-trifluoromethyl-benzenesulfonyl)-piperidin-4-yl]-6-(2,2,2-trifluoro-ethoxy)-N*4*-(3-trifluoromethyl-phenyl)-1,3,5-triazine-2,4-diamine

Yield 248 mg, 79%. ¹H-NMR (400 MHz, DMSO-D₆) δ_{H}: 1.58 (2H, m), 1.94 (2H, m), 2.88-3.01 (2H, two broad m, Z/E forms), 3.80 (2H, broad m), 3.98 (1H, broad peak, Z/E forms), 4.95 (2H, q, J=7.5 Hz), 7.30 (1H, broad d, J=8.5 Hz), 7.50 (1H, broad t, J=8.5 Hz), 7.72 (1H, broad peak, Z/E forms), 7.88-7.98 (1H, broad, Z/E forms), 8.00 (1H, d, J=8.5 Hz), 8.08 (1H, broad peak, Z/E forms), 8.18 (1H, broad peak, Z/E forms), 8.18-8.33 (1H, two broad peaks, Z/E forms), 9.73-9.95 (1H, two broad peaks, Z/E forms). MW 679.0. LCMS t_{R} (min): 2.24. MS (APCI+), m/z 679.05, 681.03 [M+H]⁺. HPLC t_{R} (min): 18.96. M_{P} 185-186° C.

### 8. N-[1-(4-Chloro-benzenesulfonyl)-piperidin-4-yl]-6-(2,2,2-trifluoro-ethoxy)-N′-(3-trifluoromethyl-phenyl)-[1,3,5]triazine-2,4-diamine

Yield 274 mg, 78%. ¹H-NMR (400 MHz, DMSO-D₆) δ_{H}: 1.61 (2H, m), 1.91 (2H, m), 2.42 (2H, m), 3.61 (2H, broad peak, Z/E forms), 3.81 (1H, broad peak, Z/E forms), 4.92 (1H, superposition of two q, J=7.5 Hz, Z/E forms), 7.29 (1H, d, J=8.5 Hz), 7.48 (1H, superposition of two m, Z/E forms), 7.71 (5H, superposition of two m, Z/E forms), 7.71-7.91 (1H, two broad peaks, Z/E forms), 8.11-8.39 (1H, two broad peaks, Z/E forms), 9.69-9.91 (1H, two broad peaks, Z/E forms). MW 610.93. LCMS t_{R} (min): 2.22. MS (APCI+), m/z 610.90, 612.96 [M+H]⁺. HPLC t_{R} (min): 18.27. M_{P} 208-210° C.

### 9. N-[1-(3-Fluoro-benzenesulfonyl)-piperidin-4-yl]-6-(2,2,2-trifluoro-ethoxy)-N′-(3-trifluoromethyl-phenyl)-[1,3,5]triazine-2,4-diamine

Yield 192 mg, 65%. ¹H-NMR (400 MHz, DMSO-D₆) δ_{H}: 1.60 (2H, m), 1.92 (2H, m), 2.48 (2H, m), 3.62 (2H, m), 3.80 (1H, broad peaks, Z/E forms), 4.92 (2H, broad q, J=7.5 Hz), 7.28 (1H, t, J=8.5 Hz), 7.47 (1H, superposition of two m, Z/E forms), 7.57 (1H, d, J=8.5 Hz), 7.61 (3H, m), 7.72 (1H, m), 7.82-7.98 (1H, two broad peaks, Z/E forms), 8.09-8.33 (1H, two broad peaks, Z/E forms), 9.68-9.90 (1H, two broad peaks, Z/E forms). MW 594.51. LCMS t_{R} (min): 2.12. MS (APCI+), m/z 595.05 [M+H]⁺. HPLC t_{R} (min): 18.06. M_{P} 199-200° C.

### 10. 1-(5-{4-[4-(2,2,2-Trifluoro-ethoxy)-6-(3-trifluoromethyl-phenylamino)-[1,3,5]triazin-2-ylamino]-piperidine-1-sulfonyl}-2,3-dihydro-indol-1-yl)-propan-1-one

Yield 231 mg, 69%. ¹H-NMR (400 MHz, DMSO-D₆) δ_{H}: 1.10 (3H, broad peak, Z/E forms), 1.60 (4H, m), 1.90 (4H, m), 2.38 (2H, m), 3.20 (2H, m), 3.58 (2H, m), 3.77 (1H, broad peak, Z/E forms), 4.17 (2H, broad peak, Z/E forms), 4.90 (2H, superposition of two q, J=7.5 Hz, Z/E forms), 7.27 (1H, superposition of two m, Z/E forms), 7.44 (1H, superposition of two m, Z/E forms), 7.53 (2H, m), 7.65-7.82 (1H, two broad peaks, Z/E forms), 7.97-8.08 (1H, two broad peaks, Z/E forms), 8.22 (1H, broad peak, Z/E forms), 8.29 (1H, broad peak, Z/E forms), 9.70-9.90 (1H, two broad peaks, Z/E forms). MW 673.64. LCMS t_{R} (min): 2.07. MS (APCI+), m/z 674.11 [M+H]⁺. HPLC t_{R} (min): 17.37. M_{P} 238-239° C.

### 11. N-[1-(4-Nitro-benzenesulfonyl)-piperidin-4-yl]-6-(2,2,2-trifluoro-ethoxy)-N′-(3-trifluoromethyl-phenyl)-[1,3,5]triazine-2,4-diamine

Yield 1.32 g, 93%. ¹H-NMR (400 MHz, DMSO-D₆) δ_{H}: 1.58 (2H, m), 1.90 (2H, m), 2.48-2.62 (2H, broad peak, Z/E forms), 3.78 (2H, m), 3.80 (1H, broad peak, Z/E forms), 4.91 (2H, superposition of two broad q, J=7.5 Hz, Z/E forms), 7.24-7.30 (1H, two d, J=7.5 Hz, Z/E forms), 7.45 (1H, superposition of two t, J=7.5 Hz, Z/E forms), 7.64 (1H, broad peak, Z/E forms), 7.83-7.96 (1H, two broad peaks, Z/E forms), 8.03 (2H, d, J=8.5 Hz), 8.32-8.42 (1H, two broad peaks, Z/E forms), 8.46 (2H, d, J=8.5 Hz), 9.70-9.92 (1H, two broad peaks, Z/E forms). MW 621.52. LCMS t_{R} (min): 2.26. MS (APCI+), m/z 622.08 [M+H]⁺. HPLC t_{R} (min): 17.75. M_{P} 238-240° C.

### 12. N-[1-(4-Amino-benzenesulfonyl)-piperidin-4-yl]-6-(2,2,2-trifluoro-ethoxy)-N′-(3-trifluoromethyl-phenyl)-[1,3,5]triazine-2,4-diamine

Yield 790 mg, 83%. ¹H-NMR (400 MHz, DMSO-D₆) δ_{H}: 1.58 (2H, m), 1.90 (2H, m), 2.30 (2H, m), 3.50 (2H, m), 3.72 (1H, broad peaks, Z/E forms), 4.92 (2H, broad q, J=7.5 Hz), 5.95 (2H, broad peaks, Z/E forms), 6.68 (2H, d, J=8.5 Hz), 7.25-7.32 (1H, two broad peaks, Z/E forms), 7.35 (2H, d, J=8.5 Hz, Z/E forms), 7.44 (1H, superposition of two t, J=8.5 Hz, Z/E forms), 7.62-7.71 (1H, two broad peaks, Z/E forms), 7.80-7.98 (1H, two broad peaks, Z/E forms), 8.08-8.28 (1H, two broad peaks, Z/E forms), 9.70-9.90 (1H, two broad peaks, Z/E forms). MW 591.53. LCMS t_{R} (min): 2.13. MS (APCI+) m/z 592.09 [M+H]⁺. HPLC t_{R} (min) 16.07. M_{P} 215-217° C.

### 13. N-[1-(3-Amino-benzenesulfonyl)-piperidin-4-yl]-6-(2,2,2-trifluoro-ethoxy)-N′-(3-trifluoromethyl-phenyl)-[1,3,5]triazine-2,4-diamine

Yield 220 mg, 78%. ¹H-NMR (400 MHz, DMSO-D₆) δ_{H}: 1.58 (2H, m), 1.92 (2H, m), 2.42 (2H, m), 3.55 (2H, m), 3.78 (1H, broad peak, Z/E forms), 4.92 (2H, broad q, J=7.5 Hz), 5.52 (2H, broad peak, Z/E forms), 6.82 (2H, m), 6.93 (1H, s), 7.22 (1H, m), 7.30 (1H, broad peak, Z/E forms), 7.43 (1H, m), 7.62-7.72 (1H, two broad peaks, Z/E forms), 7.80-7.98 (1H, two broad peaks, Z/E forms), 8.09-8.27 (1H, two broad peaks, Z/E forms), 9.65-9.92 (1H, two broad peaks, Z/E forms). MW 591.54. LCMS t_{R} (min): 1.98. MS (APCI+), m/z 592.09 [M+H]⁺. HPLC t_{R} (min): 16.19. M_{P} 195-197° C.

### 14. N-(4-{4-[4-(2,2,2-Trifluoro-ethoxy)-6-(3-trifluoromethyl-phenylamino)-[1,3,5]triazin-2-ylamino]-piperidine-1-sulfonyl}-phenyl)-acetamide

A mixture of compound 12 (220 mg, 0.37 mmol), acetic anhydride (57 mg, 0.56 mmol), DIPEA (72 mg, 0.56 mmol), acetonitrile (10 mL) was stirred at 50° C. for 6 hours, diluted with water. The formed precipitate was filtered off, washed with water and ether giving compound 14 as white crystals.

Yield 122 mg, 52%. ¹H-NMR (400 MHz, DMSO-D₆) δ_{H}: 1.63 (2H, m), 1.92 (2H, m), 2.15 (3H, s), 2.42 (2H, m), 3.61 (2H, broad peak, Z/E forms), 3.81 (1H, broad peak, Z/E forms), 4.92 (2H, broad q, J=7.5 Hz), 7.25 (1H, broad peak, Z/E forms), 7.51 (1H, broad peak, Z/E forms), 7.72 (1H, broad peak, Z/E forms), 7.82 (1H, broad peak, Z/E forms), 7.82-8.01 (1H, two broad peaks, Z/E forms), 8.05-8.31 (1H, two broad peaks, Z/E forms), 9.71-9.91 (1H, two broad peaks, Z/E forms), 10.31 (1H, broad peak, Z/E forms). MW 633.57. LCMS t_{R} (min): 2.00. MS (APCI+) m/z 633.65 [M+H]⁺. HPLC t_{R} (min) 15.17. M_{P} 180-182° C.

### 15. N-(3-{4-[4-(2,2,2-Trifluoro-ethoxy)-6-(3-trifluoromethyl-phenylamino)-[1,3,5]-triazin-2-ylamino]-piperidine-1-sulfonyl}-phenyl)-acetamide

A mixture of 13 (150 mg, 0.25 mmol), DIPEA (0.2 ml) and acetic anhydride (200 mg, 2 mmol) in acetonitrile (6 ml) was stirred at room temperature for 18 hours. The formed precipitate was filtered, washed with acetonitrile/water and aqueous K₂CO₃ giving compound 15 as a cream powder.

Yield 105 mg (66%). ¹H-NMR (400 MHz, DMSO-D₆) δ_{H}: 1.60 (2H, m), 1.92 (2H, m), 2.10 (3H, s), 2.42 (2H, m), 3.60 (2H, m), 3.80 (1H, broad peaks, Z/E forms), 4.92 (2H, broad q, J=7.5 Hz), 7.27 (1H, superposition of two d, J=8.5 Hz, Z/E forms), 7.38 (1H, d, J=8.5 Hz), 7.45 (1H, broad peak, Z/E forms), 7.57 (1H, t, J=8.5 Hz), 7.62-7.72 (1H, two broad peaks, Z/E forms), 7.85 (2H, broad peak, Z/E forms), 7.98-8.06 (1H, two broad peaks, Z/E forms), 8.09-8.28 (1H, two broad peaks, Z/E forms), 9.70-9.90 (1H, two broad peaks, Z/E forms), 10.22 (1H, broad peak, Z/E forms). LCMS t_{R} (min): 2.02. MS (APCI+), m/z 633.97 [M+H]⁺. HPLC t_{R} (min): 16.00. Mp 142-144° C.

### 16. N-[1-(3,4-Dimethyl-benzenesulfonyl)-piperidin-4-yl]-6-(2,2,2-trifluoro-ethoxy)-N′-(3-trifluoromethyl-phenyl)-[1,3,5]triazine-2,4-diamine

Yield 280 mg, 93%. MS (APCI+), m/z 605 [M+H]⁺.

### 17. [4-(1-Benzenesulfonyl-piperidin-4-yloxy)-6-(2,2,2-trifluoro-ethoxy)-[1,3,5]-triazin-2-yl]-(3-trifluoromethyl-phenyl)-amine

Yield 150 mg, 39%. ¹H-NMR (400 MHz, DMSO-D₆) δ_{H}: 1.82 (2H, m), 2.05 (2H, m), 2.95 (2H, m), 3.20 (2H, m), 4.98 (2H, q, J=7.5 Hz, Z/E forms), 5.09 (1H, broad peak, Z/E forms), 7.38 (1H, d, J=8.5 Hz), 7.54 (1H, d, J=8.5 Hz), 7.67 (2H, m), 7.77 (4H, m), 8.10 (1H, broad peak, Z/E forms), 10.42 (1H, broad peak, Z/E forms). MW 577.51. LCMS t_{R} (min): 2.13. MS (APCI+), m/z 578.13 [M+H]⁺. MS (APCI−), m/z 575.99 [M−H]. HPLC t_{R} (min): 18.02. M_{P} 96-99° C.

### 18. N-[1-(4-Fluoro-3-trifluoromethyl-benzenesulfonyl)-piperidin-4-yl]-6-(2,2,2-trifluoro-ethoxy)-N′-(3-trifluoromethyl-phenyl)-[1,3,5]triazine-2,4-diamine

To a solution of N-Piperidin-4-yl-6-(2,2,2-trifluoro-ethoxy)-N′-(3-trifluoromethyl-phenyl)-[1,3,5]triazine-2,4-diamine (200 mg, 0.46 mmol) in acetone (5 mL) DIPEA (0.2 mL) and then a solution of 4-fluoro-3-trifluoromethyl-benzenesulfonyl chloride (150 mg, 0.57 mmol) in acetone (2 mL) were added. The reaction mixture was stirred at room temperature for 2 hours, diluted with water and filtered. The precipitate was crystallized from dichloromethane giving a final product as white powder. Yield 250 mg, 92%. ¹H-NMR (400 MHz, DMSO-D₆) δ_{H}: 1.60 (2H, m), 1.93 (2H, m), 2.60 (2H, m), 3.70 (2H, m), 3.82 (1H, broad peak, Z/E forms), 4.92 (2H, broad q, J=7.5 Hz), 7.28 (1H, broad peak, Z/E forms), 7.48 (1H, broad peak, Z/E forms), 7.62 (1H, broad), 7.82 (2H, broad peak, Z/E forms), 7.99 (1H, broad peak, Z/E forms), 8.05-8.18 (1H, two broad peaks, Z/E forms), 8.18-8.38 (1H, two broad peaks, Z/E forms), 9.70-9.90 (1H, two broad peaks, Z/E forms). MW 662.51. LCMS t_{R} (min): 2.21. MS (APCI+), m/z 663.10 [M+H]⁺. HPLC t_{R} (min): 18.62. Mₚ 250-252° C.

### 19. N-[1-(2-Fluoro-benzenesulfonyl)-piperidin-4-yl]-6-(2,2,2-trifluoro-ethoxy)-N′-(3-trifluoromethyl-phenyl)-[1,3,5]triazine-2,4-diamine

Yield 300 mg, 75%. ¹H-NMR (400 MHz, DMSO-D₆) δ_{H}: 1.58 (2H, m), 1.92 (2H, m), 2.62-2.78 (2H, two m, Z/E forms), 3.70 (2H, m), 3.90 (1H, broad peak, Z/E forms), 4.95 (2H, broad q, J=7.5 Hz), 7.29 (1H, broad t, J=8.5 Hz), 7.47 (3H, broad m), 7.68 (1H, broad peak, Z/E forms), 7.78 (2H, m), 7.85-7.98 (1H, two broad peaks, Z/E forms), 8.08-8.34 (1H, two broad peaks, Z/E forms), 9.72-9.95 (1H, two broad peaks, Z/E forms). MW 594.51. LCMS t_{R} (min): 2.12. MS (APCI+), m/z 594.96 [M+H]⁺. HPLC t_{R} (min): 17.65. Mₚ 171-173° C.

### 20. N-[1-(Pyridine-3-sulfonyl)-piperidin-4-yl]-6-(2,2,2-trifluoro-ethoxy)-N′-(3-trifluoromethyl-phenyl)-[1,3,5]triazine-2,4-diamine

To a mixture of compound 17 (400 mg, 0.92 mmol) and NEt₃ (139 mg, 1.37 mmol) in acetonitrile (10 mL) a pyridine-3-sulfonyl chloride (163 mg, 0.92 mmol) was added. The reaction mixture was stirred at room temperature for 3 hours, diluted with 30% aqueous solution of NaOH and the formed solid was collected by filtration, washed with water, hexane. The product was purified by column chromatography on silica gel (15% ethyl acetate/dichloromethane) and reprecipitated from DMSO/H₂O to give a final compound as white crystals. Yield 87 mg, 17%. ¹H-NMR (400 MHz, DMSO-D₆) δ_{H}: 1.61 (2H, m), 2.05 (2H, m), 2.72 (2H, m), 3.68 (2H, m), 3.81 (1H, broad peak, Z/E forms), 4.95 (2H, broad q, J=7.5 Hz), 7.21 (1H, broad peak, Z/E forms), 7.48 (1H, broad peak, Z/E forms), 7.72 (2H, broad peak, Z/E forms), 7.81-7.98 (1H, two broad peaks, Z/E forms), 8.11-8.18 (1H, two broad peaks, Z/E forms), 8.18-8.31 (1H, two broad peaks, Z/E forms), 8.31 (2H, broad peak, Z/E forms), 9.71-9.91 (1H, two broad peaks, Z/E forms). MW 577.51. LCMS t_{R} (min): 1.97. MS (APCI+), m/z 577.96 [M+H]⁺. HPLC t_{R} (min): 16.28. Mₚ 222-224° C.

### 21. N-[1-(3-Nitro-benzenesulfonyl)-piperidin-4-yl]-6-(2,2,2-trifluoro-ethoxy)-N′-(3-trifluoromethyl-phenyl)-[1,3,5]triazine-2,4-diamine

Yield 566 mg, 91%. ¹H-NMR (400 MHz, DMSO-D₆) δ_{H}: 1.60 (2H, m), 1.92 (2H, m), 2.50 (1H, m) 2.62 (1H, m), 3.70 (2H, m), 3.80 (1H, broad peak, Z/E forms), 4.92 (2H, superposition of two q, J=8.5 Hz, Z/E forms), 7.25-7.30 (1H, superposition of two d, J=8.5 Hz, Z/E forms), 7.44-7.50 (1H, superposition of two t, J=8.5 Hz, Z/E forms), 7.64 (1H, broad peak, Z/E forms), 7.80-7.96 (1H, two broad peaks, Z/E forms), 7.98-8.12 (1H, two broad peaks, Z/E forms), 8.18 (1H, m), 8.20-8.30 (1H, two broad peaks, Z/E forms), 8.40 (1H, broad peak, Z/E forms), 8.51-8.59 (1H, two broad d, J=8.5 Hz, Z/E forms), 9.70-9.90 (1H, two broad peaks, Z/E forms). MW 621.5221. LCMS t_{R} (min): 2.10. MS (APCI+), m/z 621.87 [M+H]⁺. HPLC t_{R} (min): 17.55. M_{P} 225-227° C.

### 22. N-[1-(4-Dimethylamino-benzenesulfonyl)-piperidin-4-yl]-6-(2,2,2-trifluoro-ethoxy)-N′-(3-trifluoromethyl-phenyl)-[1,3,5]triazine-2,4-diamine

To a suspension of compound 22 (300 mg, 0.51 mmol) in dichloroethane (25 mL), acetone (88 mg, 1.52 mmol), acetic acid (0.02 mL) and NaHB(OAc)₃ (430 mg, 2.03 mmol) were added. The reaction mixture was stirred at room temperature for 5 days. The resulting mixture was poured into aqueous NaHCO₃, stirred for 3 hours and extracted with dichloromethane (2×30 mL). The combined organic phases were washed with water, dried over sodium sulfate and concentrated. The residue was purified by column chromatography on silica gel (10% ethyl acetate/dichloromethane) and triturated with hexane giving a final compound as white crystals.

Yield 110 mg, 51%. ¹H-NMR (400 MHz, DMSO-D₆) δ_{H}: 1.19 (6H, broad d, J=7.5 Hz), 1.58 (2H, m), 1.90 (2H, m), 2.33 (2H, m), 3.50 (2H, m), 3.62 (1H, broad peak. Z/E forms), 3.77 (1H, broad peak. Z/E forms), 4.92 (2H, broad q, J=7.5 Hz), 6.33 (1H, broad peak. Z/E forms), 6.65 (2H, d, J=8.5 Hz), 7.27 (1H, broad m), 7.38 (2H, d, J=8.5 Hz), 7.45 (1H, m), 7.60-7.70 (1H, broad peak. Z/E forms), 7.78-7.97 (1H, two broad peaks. Z/E forms), 8.08-8.28 (1H, two broad peaks. Z/E forms), 9.68-9.88 (1H, two broad peaks. Z/E forms). MW 633.62. LCMS t_{R} (min): 2.14. MS (APCI+), m/z 633.97 [M+H]⁺ HPLC t_{R} (min): 17.47. Mₙ 190-192° C.

### 23. N-(3-{4-[4-(2,2,2-Trifluoro-ethoxy)-6-(3-trifluoromethyl-phenylamino)-[1,3,5]triazin-2-ylamino]-piperidine-1-sulfonyl}-phenyl)-methanesulfonamide

To a solution of compound 22 (200 mg, 0.34 mmol) and DIPEA (100 mg, 0.78 mmol) in THF (5 mL) methanesulfonyl chloride (90 mg, 0.78 mmol) was added. The reaction mixture was refluxed for 6 hours, the solvent was evaporated and the residue was dissolved in 2,2,2-trifluoroethanol (3 mL), NaOH (100 mg) was added and stirred at 60° C. for 30 minutes. The mixture was concentrated, dissolved in dichloromethane (50 mL) and washed with 3% aqueous solution of HCl (2×10 mL). The organic layer dried over Na₂SO₄ and concentrated. Purification by column chromatography (dichloromethane/acetone, 10/1) and reprecipitation from methanol/water, then from DMSO/water gave compound 24.

Yield 90 mg, 42%. ¹H-NMR (400 MHz, DMSO-D₆) δ_{H}: 1.60 (2H, m), 1.92 (2H, m), 3.05 (3H, s), 3.20 (2H, m), 3.60 (2H, m), 3.80 (1H, broad peak. Z/E forms), 4.92 (2H, broad q, J=7.5 Hz), 7.28 (1H, broad t, J=8.5 Hz), 7.44 (2H, broad peak. Z/E forms), 7.53 (1H, broad peak. Z/E forms), 7.58 (2H, broad peak, Z/E forms), 7.60-7.69 (1H, two broad peaks. Z/E forms), 7.80-7.98 (1H, two broad peaks. Z/E forms), 8.07-8.28 (1H, two broad peaks. Z/E forms), 9.70-9.90 (1H, two broad peaks. Z/E forms), 10.10 (1H, broad peak. Z/E forms). MW 669.63. LCMS t_{R} (min): 1.97. MS (APCI+), m/z 667.80, 669.80 [M+H]⁺. HPLC t_{R} (min): 16.23. Mₚ. 145-148° C.

### 24. N-[1-(4-Nitro-benzenesulfonyl)-piperidin-4-yl]-6-(2,2,2-trifluoro-ethoxy)-N′-(3-trifluoromethyl-phenyl)-[1,3,5]triazine-2,4-diamine

Yield 1.32 g, 93%. ¹H-NMR (400 MHz, DMSO-D₆) δ_{H}: 1.58 (2H, m), 1.90 (2H, m), 2.48-2.62 (2H, broad peak. Z/E forms), 3.78 (2H, m), 3.80 (1H, broad peak, Z/E forms), 4.91 (2H, superposition of two broad q, J=7.5 Hz, Z/E forms), 7.24-7.30 (1H, two d, J=7.5 Hz, Z/E forms), 7.45 (1H, superposition of two t, J=7.5 Hz, Z/E forms), 7.64 (1H, broad peak. Z/E forms), 7.83-7.96 (1H, two broad peaks. Z/E forms), 8.03 (2H, d, J=8.5 Hz), 8.32-8.42 (1H, two broad peaks. Z/E forms), 8.46 (2H, d, J=8.5 Hz), 9.70-9.92 (1H, two broad peaks, Z/E forms). MW 621.52. LCMS t_{R} (min): 2.26. MS (APCI+), m/z 622.08 [M+H]⁺. HPLC t_{R} (min): 17.75. M_{P} 238-240° C.

### 25. N-[1-(4-Dimethylamino-benzenesulfonyl)-piperidin-4-yl]-6-(2,2,2-trifluoro-ethoxy)-N′-(3-trifluoromethyl-phenyl)-[1,3,5]triazine-2,4-diamine

To a solution of the nitro precursor (1 g, 1.61 mmol) and hydrazine hydrate (447 mg, 8.93 mmol) in THF (30 mL) a suspension of Ra—Ni in water (600 mg, 10.22 mmol) was added dropwise at room temperature. The mixture was stirred at 50° C. for 1 hour, filtered and concentrated. The residue was purified by column chromatography on silica gel (chloroform/ethyl acetate, 5/1) giving the amino intermediate as cream crystals. Yield 790 mg, 83%. ¹H-NMR (400 MHz, DMSO-D₆) δ_{H}: 1.58 (2H, m), 1.90 (2H, m), 2.30 (2H, m), 3.50 (2H, m), 3.72 (1H, broad peaks. Z/E forms), 4.92 (2H, broad q, J=7.5 Hz), 5.95 (2H, broad peaks. Z/E forms), 6.68 (2H, d, J=8.5 Hz), 7.25-7.32 (1H, two broad peaks. Z/E forms), 7.35 (2H, d, J=8.5 Hz, Z/E forms), 7.44 (1H, superposition of two t, J=8.5 Hz, Z/E forms), 7.62-7.71 (1H, two broad peaks. Z/E forms), 7.80-7.98 (1H, two broad peaks. Z/E forms), 8.08-8.28 (1H, two broad peaks. Z/E forms), 9.70-9.90 (1H, two broad peaks. Z/E forms). MW 591.53. LCMS t_{R} (min): 2.13. MS (APCI+) m/z 592.09 [M+H]⁺. HPLC t_{R} (min) 16.07. M_{P} 215-21° C.

To a suspension of the amino intermediate (300 mg, 0.51 mmol) in dichloroethane (25 mL), acetone (88 mg, 1.52 mmol), acetic acid (0.02 mL) and NaHB(OAc)₃ (430 mg, 2.03 mmol) were added. The reaction mixture was stirred at room temperature for 36 hours. The resulting mixture was poured into aqueous NaHCO₃, stirred for 3 hours and extracted with dichloromethane (2×30 mL). The combined organic phases were washed with water, dried over sodium sulfate and concentrated. The residue was purified by column chromatography on silica gel (10% ethyl acetate/dichloromethane) and triturated with hexane giving the compound as white crystals. Yield 200 mg, 62%. ¹H-NMR (400 MHz, DMSO-D₆) δ_{H}: 1.19 (6H, broad d, J=7.5 Hz), 1.58 (2H, m), 1.90 (2H, m), 2.33 (2H, m), 3.50 (2H, m), 3.62 (1H, broad peak, Z/E forms), 3.77 (1H, broad peak, Z/E forms), 4.92 (2H, broad q, J=7.5 Hz), 6.33 (1H, broad peak. Z/E forms), 6.65 (2H, d, J=8.5 Hz), 7.27 (1H, broad m), 7.38 (2H, d, J=8.5 Hz), 7.45 (1H, m), 7.60-7.70 (1H, broad peak. Z/E forms), 7.78-7.97 (1H, two broad peaks, Z/E forms), 8.08-8.28 (1H, two broad peaks, Z/E forms), 9.68-9.88 (1H, two broad peaks, Z/E forms). MW 633.62. LCMS t_{R} (min): 2.14. MS (APCI+), m/z 633.97 [M+H]⁺ HPLC t_{R} (min): 17.47. Mₚ 190-192° C.

### 26. N,N-Dimethyl-4-{4-[4-(2,2,2-trifluoro-ethoxy)-6-(3-trifluoromethyl-phenylamino)-[1,3,5]triazin-2-ylamino]-piperidine-1-sulfonyl}-benzamide

¹H-NMR (400 MHz, DMSO-D₆) δ_{H}: 1.60 (2H, broad peak, Z/E forms), 1.92 (2H, broad peak, Z/E forms), 2.49 (2H, m), 2.95 (3H, broad peak, Z/E forms), 3.19 (3H, broad peak, Z/E forms), 3.63 (2H, m), 3.81 (1H, broad peak, Z/E forms), 4.92 (2H, broad q, J=7.5 Hz), 7.28 (1H, broad t, J=8.5 Hz), 7.41-7.52 (1H, two broad d, J=8.5 Hz, Z/E forms), 7.64 (3H, m), 7.79 (2H, m), 7.81-7.97 (1H, two broad peaks, Z/E forms), 8.07-8.28 (1H, two broad peaks, Z/E forms), 9.70-9.90 (1H, two broad peaks, Z/E forms). MW 647.60. LCMS t_{R} (min): 1.97. MS (APCI+), m/z 648.50 [M+H]⁺. HPLC t_{R} (min): 16.14. M_{P} 178-179° C.

### 27. N-(1-Methanesulfonyl-piperidin-4-yl)-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 1.60 (2H, m), 1.97 (2H, m), 2.83 (2H, m), 2.88 (3H, s), 3.60 (2H, m), 3.93 (1H, broad peak, Z/E forms), 4.95 (2H, broad q, J=7.5 Hz, Z/E forms), 7.31 (1H, broad d, J=8.5 Hz, Z/E forms), 7.52 (1H, superposition of two t, J=8.5 Hz, Z/E forms), 7.66-7.82 (1H, two broad d, J=8.5 Hz, Z/E forms), 7.80-8.00 (1H, two broad peaks, Z/E forms), 8.10-8.28 (1H, two broad peaks, Z/E forms), 9.71-9.93 (1H, two broad peaks, Z/E forms). MW 514.45. LCMS t_{R} (min): 1.93. MS (APCI+), m/z 514.98 (100) [M+H]⁺. MS (APCI−), m/z 512.88 (100) [M−H]⁻,

### 28. N-[1-(4-Dimethylamino-benzenesulfonyl)-piperidin-4-yl]-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 1.60 (2H, m), 1.90 (2H, m), 2.34 (2H, m), 3.03 (6H, s), 3.52 (2H, broad peak, Z/E forms), 3.75 (1H, broad peak, Z/E forms), 4.92 (2H, broad q, J=7.5 Hz), 6.82 (2H, d, J=8.5 Hz), 7.28 (1H, broad peak, Z/E forms), 7.45 (1H, broad peak, Z/E forms), 7.50 (2H, d, J=8.5 Hz), 7.60-7.67 (1H, broad, Z/E forms), 7.78-7.98 (1H, two broad peaks, Z/E forms), 8.08-8.31 (1H, two broad peaks, Z/E forms), 9.70-9.88 (1H, two broad peaks, Z/E forms). MW 619.59. LCMS t_{R} (min): 2.14. MS (APCI+), m/z 620.04 [M+H]⁺. HPLC t_{R} (min): 17.79. M_{P} 251.5-252.5° C.

### 29. N-(3-{4-[4-(2,2,2-Trifluoro-ethoxy)-6-(3-trifluoromethyl-phenylamino)-[1,3,5]triazin-2-ylamino]-piperidine-1-sulfonyl}-phenyl)-methanesulfonamide

A mixture of compound 22 (150 mg, 0.25 mmol), DIPEA (0.2 mL) and methanesulfonyl chloride (11) (148 mg, 1.28 mmol) in acetonitrile (5 mL) was stirred at room temperature for 20 hours, then a saturated aqueous solution of K₂CO₃ (5 mL) was added and the resulting mixture was stirred at room temperature for 48 hours. The formed precipitate was filtered, treated with a solution of sodium hydroxide (100 mg) in methanol (5 mL) and refluxed for 30 min. Then the mixture cooled down to room temperature and acidified with 5% aqueous solution of HCl to pH 4. The formed solid was collected by filtration, washed with water and dried to give the compound as a white powder.

Yield 120 mg, 80%. ¹H-NMR (400 MHz, DMSO-D₆) δ_{H}: 1.61 (2H, m), 1.93 (2H, m), 2.42 (2H, m), 3.11 (3H, s), 3.51 (2H, m), 3.71 (1H, broad peak, Z/E forms), 3.82 (3H, s), 7.25 (1H, broad peak, Z/E forms), 7.41 (2H, m), 7.51 (1H, m), 7.61 (2H, m), 7.71 (1H, broad peak, Z/E forms), 8.11 (1H, broad d, J=8.5 Hz), 8.31 (1H, broad peak, Z/E forms), 9.51-9.71 (1H, two broad peaks, Z/E forms), 10.21 (1H, broad peak, Z/E forms). MW 601.63. LCMS t_{R} (min): 1.84. MS (APCI+), m/z 602.09 [M+H]⁺. HPLC t_{R} (min): 14.14. M_{P} 249-251° C.

### 30. N-Piperidin-4-yl-6-(2,2,2-trifluoro-ethoxy)-N′-(3-trifluoromethyl-phenyl)-[1,3,5]triazine-2,4-diamine (23)

Yield 8.60 g, 98%. ¹H-NMR (400 MHz, DMSO-D₆) δ_{H}: 1.41 (2H, m), 1.75 (2H, m), 2.51 (2H, m), 3.11 (2H, m), 3.84 (1H, broad peak, Z/E forms), 4.95 (2H, q, J=7.5 Hz), 7.31 (1H, d, J=8.5 Hz), 7.42 (1H, t, J=8.5 Hz), 7.61-7.72 (1H, two broad peaks, Z/E forms), 7.85-7.99 (1H, two broad peaks, Z/E forms), 8.12-8.41 (1H, two broad peaks, Z/E forms), 9.71-9.95 (1H, two broad peaks, Z/E forms). MW 436.36. LCMS t_{R} (min): 1.60. MS (APCI+), m/z 437.07 [M+H]⁺. HPLC t_{R} (min): 11.37. M_{P} 168-170° C.

### 31. N-[1-(3-Bromo-benzenesulfonyl)-piperidin-4-yl]-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 1.61 (2H, m), 1.91 (2H, m), 2.49 (2H, m), 3.61 (2H, m), 3.81 (1H, broad peak, Z/E forms), 4.91 (2H, broad q, J=7.5 Hz), 7.25 (1H, superposition of two d, J=8.5 Hz, Z/E forms), 7.41 (1H, superposition of two d, J=8.5 Hz, Z/E forms), 7.59 (1H, t, J=8.5 Hz, Z/E forms), 7.61 (1H, broad d, J=8.5 Hz, Z/E forms), 7.79 (1H, broad d, J=8.5 Hz, Z/E forms), 7.80 (1H, broad d, J=8.5 Hz, Z/E forms), 7.82 (1H, broad s), 7.85-7.99 (1H, two broad peaks, Z/E forms), 8.05-8.31 (1H, two broad peaks, Z/E forms), 9.61-9.91 (1H, two broad peaks, Z/E forms). MW 655.42. LCMS t_{R} (min): 2.18. MS (APCI+), m/z 656.85, 658.04 [M+H]⁺. HPLC t_{R} (min): 18.69. M_{P} 216-217° C.

### 32. N-[1-(3-Chloro-benzenesulfonyl)-piperidin-4-yl]-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 1.61 (2H, m), 1.98 (2H, m), 2.51 (2H, m), 3.61 (2H, m), 3.81 (1H, broad peak, Z/E forms), 4.92 (2H, broad q, J=7.5 Hz), 7.29 (1H, m), 7.42 (1H, m), 7.75 (4H, m), 7.81 (1H, m), 7.81-7.91 (1H, two broad peaks, Z/E forms), 8.11-8.39 (1H, two broad peaks, Z/E forms), 9.65-9.91 (1H, two broad peaks, Z/E forms). MW 610.97. LCMS t_{R} (min): 2.17. MS (APCI+), m/z 610.92, 612.96 [M+H]⁺. HPLC t_{R} (min): 18.49. M_{P} 207-208° C.

### 33. N-[1-(3-Chloro-2-methyl-benzenesulfonyl)-piperidin-4-yl]-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 1.52 (2H, m), 1.92 (2H, m), 2.61 (3H, s), 2.72-2.91 (2H, two m, Z/E forms), 3.62 (2H, m), 3.91 (1H, broad peak, Z/E forms), 4.92 (2H, q, J=7.5 Hz), 7.31 (1H, d, J=8.5 Hz), 7.48 (2H, m), 7.61-7.75 (1H, two broad peaks, Z/E forms), 7.75 (1H, t, J=8.5 Hz), 7.81 (1H, d, J=8.5 Hz), 7.81-8.01 (1H, two broad peaks, Z/E forms), 8.11-8.35 (1H, two broad peaks, Z/E forms), 9.71-9.95 (1H, two broad peaks, Z/E forms). MW 625.00. LCMS t_{R} (min): 2.24. MS (APCI+), m/z 624.94, 626.99 [M+H]⁺. HPLC t_{R} (min): 19.11. M_{P} 202-203° C.,

### 34. N-[1-(3-Methoxy-benzenesulfonyl)-piperidin-4-yl]-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 1.61 (2H, m), 1.91 (2H, m), 2.41 (2H, m), 3.62 (2H, m), 3.78 (1H, broad peak, Z/E forms), 3.91 (3H, s), 4.92 (2H, broad q, J=7.5 Hz), 7.18 (1H, broad peak, Z/E forms), 7.28 (3H, broad peak, Z/E forms), 7.45 (1H, t, J=8.5 Hz), 7.59 (1H, t, J=8.5 Hz), 7.69 (1H, broad peak, Z/E forms), 7.71-7.98 (1H, two broad peaks, Z/E forms), 8.15-8.41 (1H, two broad peaks, Z/E forms), 9.69-9.91 (1H, two broad peaks, Z/E forms). MW 606.55. LCMS t_{R} (min): 2.10. MS (APCI+), m/z 607.00 [M+H]⁺. HPLC t_{R} (min): 17.86. M_{P} 204-205° C.

### 35. N-[1-(5-Dimethylamino-naphthalene-1-sulfonyl)-piperidin-4-yl]-6-(2,2,2-tri-fluoro-ethoxy)-N′-(3-trifluoromethyl-phenyl)-[1,3,5]triazine-2,4-diamine

Yield 92 mg, 30%. ¹H-NMR (400 MHz, DMSO-D₆) δ_{H}: 1.61 (2H, m), 1.91 (2H, m), 2.49 (1H, m), 2.69 (1H, m), 2.91 (6H, s), 3.71 (2H, m), 3.81-3.91 (1H, two broad peaks, Z/E forms), 4.92 (2H, q, J=7.5 Hz), 7.29 (2H, m), 7.41-7.51 (1H, two broad peaks, Z/E forms), 7.61 (2H, s), 7.61-7.81 (1H, two broad peaks, Z/E forms), 8.01-8.09 (1H, two broad peaks, Z/E forms), 8.15 (1H, d, J=8.5 Hz), 8.25 (1H, broad peak, Z/E forms), 8.35 (1H, d, J=8.5 Hz), 8.59 (1H, d, J=8.5 Hz), 9.71-9.91 (1H, two broad peaks, Z/E forms). MW 669.65. LCMS t_{R} (min): 2.26. MS (APCI+), m/z 670.01, 671.09 [M+H]⁺. HPLC t_{R} (min): 18.52. M_{P} 125-126° C.

### 36. 6-(2,2,2-Trifluoro-ethoxy)-N-[1-(3-trifluoromethoxy-benzenesulfonyl)-piperidin-4-yl]-N′-(3-trifluoromethyl-phenyl)-[1,3,5]triazine-2,4-diamine

Yield 227 mg, 50%. ¹H-NMR (400 MHz, DMSO-D₆) δ_{H}: 1.61 (2H, m), 1.95 (2H, m), 2.51 (2H, m), 3.61 (2H, m), 3.81 (1H, broad peak, Z/E forms), 4.92 (2H, broad q, J=7.5 Hz), 7.25-7.31 (1H, two broad d, J=8.5 Hz, Z/E forms), 7.48-7.56 (1H, two broad t, J=8.5 Hz, Z/E forms), 7.61 (2H, broad peak, Z/E forms), 7.82 (3H, broad peak, Z/E forms), 7.82-8.05 (1H, two broad peaks, Z/E forms), 8.11-8.31 (1H, two broad peaks, Z/E forms), 9.71-9.91 (1H, two broad peaks, Z/E forms). MW 660.52. LCMS t_{R} (min): 2.22. MS (APCI+), m/z 661.08 [M+H]⁺. HPLC t_{R} (min): 18.84. Mₚ 209-211° C.

### 37. N-[1-(3,4-Difluoro-benzenesulfonyl)-piperidin-4-yl]-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 1.61 (2H, m), 1.95 (2H, m), 2.45 (2H, m), 3.62 (2H, m), 3.87 (1H, broad peak, Z/E forms), 4.95 (2H, broad q, J=7.5 Hz), 7.29 (1H, broad peak, Z/E forms), 7.48 (1H, broad peak, Z/E forms), 7.68 (2H, broad peak, Z/E forms), 7.72 (1H, broad peak, Z/E forms), 7.98-8.85 (1H, two broad peaks, Z/E forms), 8.15-8.39 (1H, two broad peaks, Z/E forms), 9.71-9.98 (1H, two broad peaks, Z/E forms).

MW 612.51. LCMS t_{R} (min): 2.13. MS (APCI+), m/z 612.99 [M+H]⁺. HPLC t_{R} (min): 18.09. Mp 195-198° C.

### 38. 1-(4-{4-[4-(2,2,2-Trifluoro-ethoxy)-6-(3-trifluoromethyl-phenylamino)-[1,3,5]triazin-2-ylamino]-piperidine-1-sulfonyl}-phenyl)-pyrrolidin-2-one

¹H-NMR (400 MHz, DMSO-D₆) δ_{H}: 1.60 (2H, m), 1.90 (2H, m), 2.10 (2H, m), 2.38 (2H, m), 2.56 (2H, m), 3.60 (2H, m), 3.76 (1H, broad peak, Z/E forms), 3.90 (2H, t, J=7.5 Hz), 4.92 (2H, superposition of two quartets, J=7.5 Hz, Z/E forms), 7.28 (1H, broad m, Z/E forms), 7.45 (1H, broad m, Z/E forms), 7.62-7.69 (1H, two broad peaks, Z/E forms), 7.73 (2H, d, J=8.5 Hz), 7.80-7.92 (1H, two broad peaks, Z/E forms), 7.94 (2H, d, J=8.5 Hz), 8.09-8.28 (1H, two broad peaks, Z/E forms), 9.70-9.90 (1H, two broad peaks, Z/E forms). MW 659.62. LCMS t_{R} (min): 1.98. MS (APCI+), m/z 660.03 [M+H]⁺. HPLC t_{R} (min): 16.89. M_{P} 267-270° C.

### 39. 6-{4-[4-(2,2,2-Trifluoro-ethoxy)-6-(3-trifluoromethyl-phenylamino)-[1,3,5]triazin-2-ylamino]-piperidine-1-sulfonyl}-3,4-dihydro-1H-quinolin-2-one

Yield 170 mg, 53%. ¹H-NMR (400 MHz, DMSO-D₆) δ_{H}: 1.61 (2H, m), 1.92 (2H, m), 2.37 (2H, t, J=7.5 Hz), 2.52 (2H, m), 3.00 (2H, t, J=7.5 Hz), 3.60 (2H, m), 3.78 (1H, broad peak, Z/E forms), 4.92 (2H, superposition of two q, J=7.5 Hz, Z/E forms), 7.05 (1H, d, J=8.5 Hz), 7.27 (1H, broad peak, Z/E forms), 7.45 (1H, broad peak, Z/E forms), 7.52 (1H, d, J=8.5 Hz), 7.54 (1H, broad peak, Z/E forms), 7.61-7.67 (1H, two broad peaks, Z/E forms), 7.80-7.98 (1H, two broad peaks, Z/E forms), 8.08-8.33 (1H, two broad peaks, Z/E forms), 9.68-9.89 (1H, two broad peaks, Z/E forms), 10.37-10.40 (1H, two broad peaks, Z/E forms). MW 645.59. LCMS t_{R} (min): 1.88. MS (APCI+), m/z 646.01 [M+H]⁺. HPLC t_{R} (min): 15.68. M_{P} 265-267° C.

### 40. N-[1-(4′-Methoxy-biphenyl-3-sulfonyl)-piperidin-4-yl]-6-(2,2,2-trifluoro-ethoxy)-N′-(3-trifluoromethyl-phenyl)-[1,3,5]triazine-2,4-diamine

A mixture of compound 33 (450 mg, 0.687 mmol), 4-methoxy-phenyl-boronic acid (27) (105 mg, 0.687 mmol), Pd(PPh₃)₄ (79 mg, 0.0687 mmol) in dioxane (10 mL) was stirred at room temperature for 30 minutes, then 1M aqueous solution of Na₂CO₃ (10 mL) was added. The resulting mixture was stirred at refluxing for 3 hours, cooled down to room temperature and concentrated. The obtained residue was diluted with water and extracted with dichloromethane. The combined organic phases were concentrated. Purification by column chromatography on silica gel (hexane/ethyl acetate, 1/1.5) gave the compound. Yield 102 mg, 22%. ¹H-NMR (400 MHz, DMSO-D₆) δ_{H}: 1.61 (2H, m), 1.98 (2H, m), 2.51 (2H, m), 3.61 (2H, m), 3.80 (4H, superposition of s (3H) and broad peak (1H)), 4.91 (2H, broad q, J=7.5 Hz), 7.01 (2H, d, J=8.5 Hz), 7.21-7.31 (1H, two broad q, J=7.5 Hz, Z/E forms), 7.41-7.51 (1H, two broad t, J=7.5 Hz, Z/E forms), 7.71 (4H, m), 7.81 (2H, m), 7.92 (2H, m), 8.11-8.28 (1H, two broad peaks, Z/E forms), 9.71-9.81 (1H, two broad peaks, Z/E forms). MW 682.65. LCMS t_{R} (min): 2.22. MS (APCI+), m/z 682.98 [M+H]⁺. HPLC t_{R} (min): 18.86. M_{P} 180-182° C.

### 41. N-(1-Methanesulfonyl-piperidin-4-yl)-N-methyl-6-(2,2,2-trifluoro-ethoxy)-N′-(3-trifluoromethyl-phenyl)-[1,3,5]triazine-2,4-diamine

A solution of N-Methyl-N-(BOC)-piperidin-4-yl-6-(2,2,2-trifluoro-ethoxy)-N′-(3-trifluoromethyl-phenyl)-[1,3,5]-triazine-2,4-diamine (370 mg, 0.67 mmol) in dioxane saturated with HCl (16%, 20 mL) was stirred at room temperature for 3 hours (TLC control). Then the mixture was poured in aqueous solution of K₂CO₃ and extracted with ethyl acetate. The organic phases were dried over K₂CO₃ and concentrated. The residue oil was triturated with hexane and dried giving de-BOC-compound as a cream solid. Yield 245 mg, 81%. MW 450.39. LCMS t_{R} (min): 1.62. MS (APCI+), m/z 451.10 [M+H]⁺.

To a solution of the de-BOC-compound (240 mg, 0.53 mmol), DIPEA (89 mg, 0.69 mmol) in acetonitrile (5 mL) methanesulfonyl chloride (79 mg, 0.69 mmol) was added at room temperature. The resulting mixture was stirred at room temperature for 3 hours, diluted with water and filtered. Purification by column chromatography on silica gel (dichloromethane) and recrystallization (methanol/water) gave the compound. Yield 106 mg, 40%. ¹H-NMR (400 MHz, DMSO-D₆) δ_{H}: 1.71 (2H, m), 1.91 (2H, m), 2.81-2.91 (2H, two broad peaks, Z/E forms), 2.91 (3H, s), 2.98 (3H, s), 3.71 (2H, m), 4.61 (1H, broad peak, Z/E forms), 4.98 (2H, q, J=7.5 Hz), 7.31 (1H, broad d, J=8.5 Hz), 7.51 (1H, broad peak, Z/E forms), 7.81 (1H, d, J=8.5 Hz), 8.21-8.41 (1H, two broad peaks, Z/E forms), 9.91 (1H, broad peak, Z/E forms). MW 528.48. LCMS t_{R} (min): 2.01. MS (APCI+), m/z 529.08 [M+H]⁺. HPLC t_{R} (min): 17.06. M_{P} 161-163° C.

### 42. N-[1-(4-Morpholin-4-yl-benzenesulfonyl)-piperidin-4-yl]-6-(2,2,2-trifluoro-ethoxy)-N′-(3-trifluoromethyl-phenyl)-[1,3,5]triazine-2,4-diamine

A mixture of compound 37 (200 mg, 0.538 mmol), compound 40 (195 mg, 0.538 mmol), Et₃N (218 mg, 2.152 mmol) and acetonitrile (3 mL) was stirred at 50° C. for 4 hours, diluted with water (30 mL). The formed solid was collected by filtration, washed with water and acetonitrile and dried. Purification by crystallization from acetonitrile gave 90% pure compound (21 mg) which was sent as “crude” sample. Do we have pure MS?

### 43. N-[1-(4-Dimethylamino-benzenesulfonyl)-piperidin-4-yl]-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 1.59 (2H, m), 1.90 (2H, m), 2.31 (2H, m), 2.98 (6H, s), 3.52 (2H, broad peak, Z/E forms), 3.72 (1H, broad peak, Z/E forms), 4.92 (2H, broad q, J=7.5 Hz), 6.82 (2H, d, J=8.5 Hz), 7.28 (1H, broad peak, Z/E forms), 7.45 (1H, broad peak, Z/E forms), 7.50 (2H, d, J=8.5 Hz), 7.60-7.67 (1H, two broad peaks, Z/E forms), 7.79-7.97 (1H, two broad peaks, Z/E forms), 8.08-8.31 (1H, two broad peaks, Z/E forms), 9.68-9.88 (1H, two broad peaks, Z/E forms). MW 619.59. LCMS t_{R} (min): 2.11. MS (APCI+), m/z 620.06, 621.08 [M+H]⁺. HPLC t_{R} (min): 18.17.

### Generic Synthesis of Intermediates and Final Compounds for Table 33.

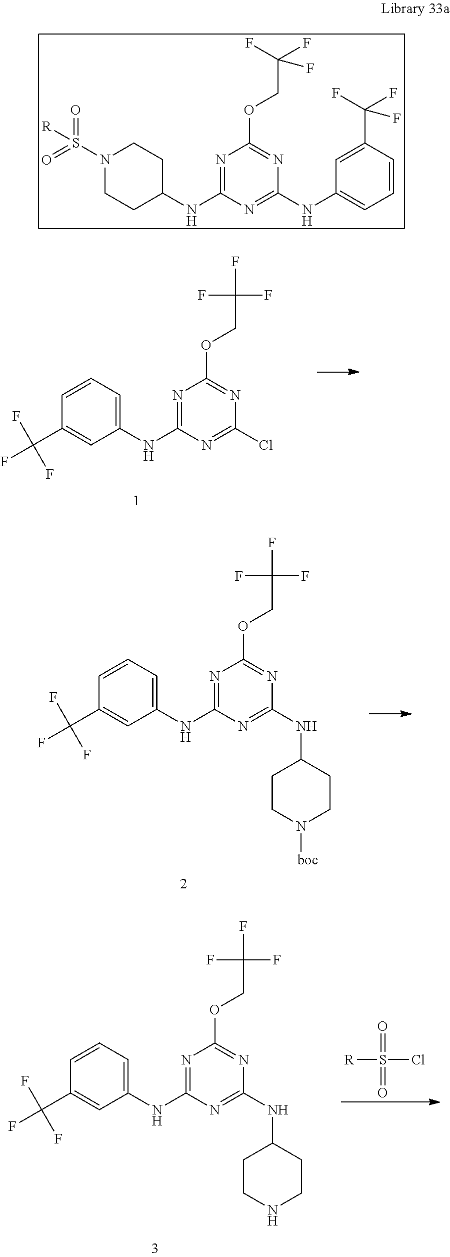

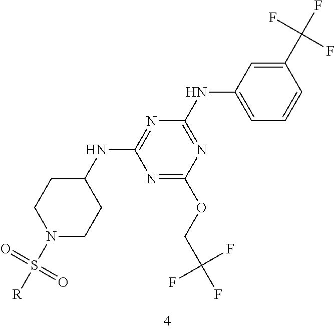

Preparation of 2 1 (10 g, 27 mmol) was dissolved in 1,4-dioxane (50 ml). 4-amino-1-Boc-piperidine (30 mmol) and triethylamine (33 mmol) were added. The reaction mixture was stirred at 80° C. overnight. Then solvent was removed under reduced pressure and the resulting precipitate was washed with water and filtered. The product was washed with ether.

Preparation of 3 2 (12 g, 23 mmol) was dissolved in 1,4-dioxane*HCl (200 ml) and stirred at room temperature overnight. Then solvent was removed under reduced pressure and the precipitate was washed with ether.

Preparation of 4 A solution of 10 mmol of 3 in 10 ml of dioxane was treated with appropriate sulphochloride (10 mmol) and triethylamine (22 mmol). This mixture was stirred at reflux for 3 hours, then cooled and evaporated. The residue was treated with water. The precipitate was filtered, washed with water, dried, and purified by column chromatography.

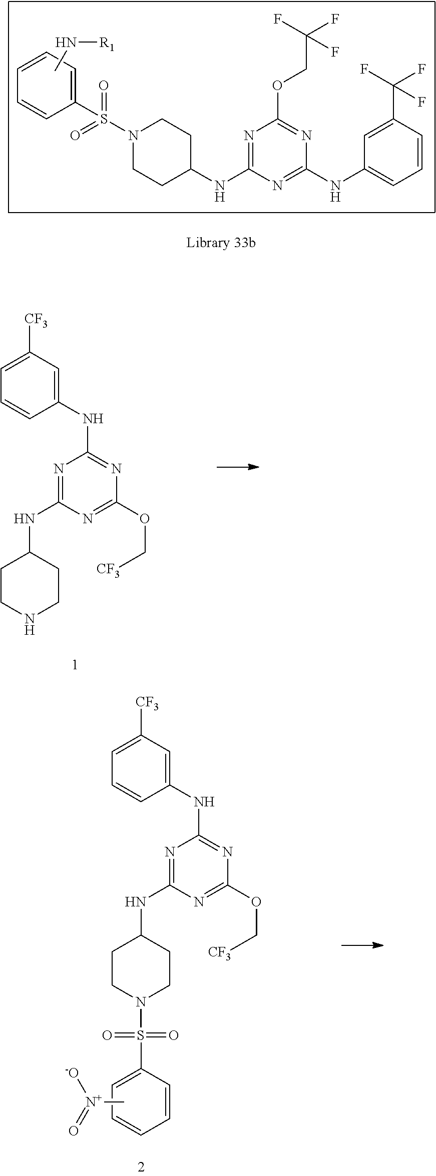

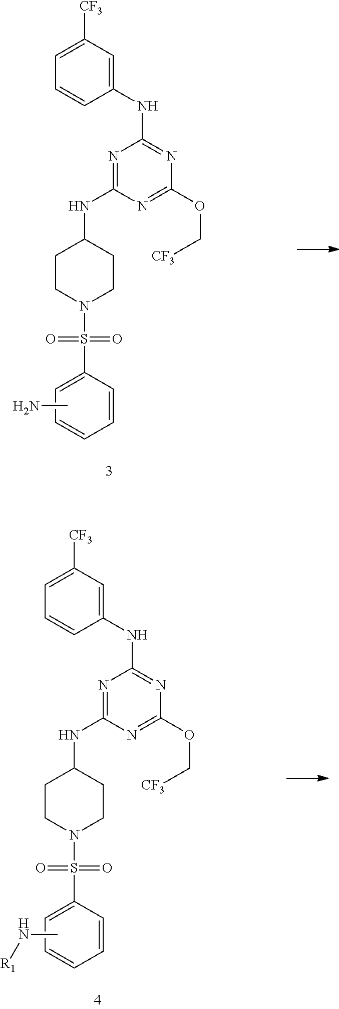

Compound 2 was obtained according to the following procedure: A solution of 10 mmol of 1 in 10 ml of dioxane was treated with sulphochloride (10 mmol) and triethylamine (22 mmol). This mixture was stirred at reflux for 3 hours, then cooled and evaporated. The residue was treated with water. The precipitate was filtered off, washed with water, dried and purified by column chromatography.

Compound 3 was obtained according to the following procedure: Compound 2 (1 g.) was dissolved in ethanol (100 ml) and hydrogenated in the presence of Pd/C catalyst under 2 atm at r.t. After completion of the reaction the catalyst was removed by filtration, the solvent was removed under reduced pressure and pure compound 3 was obtained as white solid. Yield 0.53 g.

Compound 4 Amides: Obtained according to the following procedure: A solution of 10 mmol of 3 in 10 ml of CH₂Cl₂ was treated with acyl chloride (10 mmol) and triethylamine (22 mmol). This mixture was stirred at r.t. for 3 hours, then cooled and evaporated. The residue was treated with water. The solvent was removed and purified by column chromatography. Sulfonamides: A solution of 10 mmol of 3 in 10 ml of dioxane was treated with the appropriate sulphochloride (10 mmol) and triethylamine (22 mmol). This mixture was stirred at reflux for 3 hours, then cooled and evaporated. The residue was treated with water. The precipitate formed was filtered, washed with water, dried, and purified by column chromatography. Ureas: A solution of 10 mmol of intermediate 3 in 10 ml of dioxane was treated with appropriate isocyanate (10 mmol). This mixture was stirred at reflux for 3 hours, then cooled and poured into water. The precipitate formed was filtered, washed with water and dried. The precipitate was purified by column chromatography.

TABLE 33

| En | Structure | IUPAC Name | MW | Formula |
|---|---|---|---|---|
| 1 | 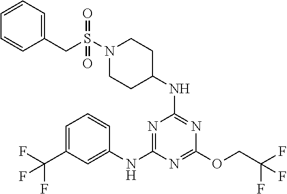 | N-[1-(benzyl- sulfonyl)piperidin- 4-yl]-6-(2,2,2- trifluoroethoxy)- N′-[3- (trifluoromethyl) phenyl]-1,3,5- triazine-2,4- diamine | 591.6 | C₂₄H₂₄F₆N₆O₃S |
| 2 | 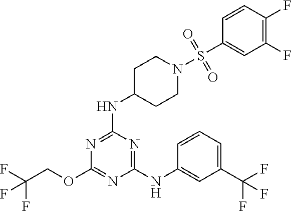 | N-{1-[(3,4- difluorophenyl) sulfonyl]piperidin- 4-yl}-6-(2,2,2- trifluoroethoxy)- N′-[3-(trifluoro- methyl)phenyl]- 1,3,5-triazine- 2,4-diamine | 613.5 | C₂₃H₂₀F₈N₆O₃S |
| 3 | 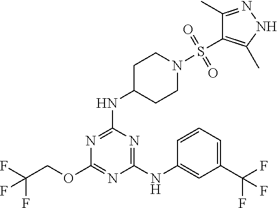 | N-{1-[(3,5- dimethyl-1H- pyrazol-4-yl) sulfonyl]piperidin- 4-yl}-6-(2,2,2- trifluoroethoxy)- N′-[3- (trifluoromethyl) phenyl]-1,3,5- triazine-2,4- diamine | 595.5 | C₂₂H₂₄F₆N₈O₃S |
| 4 | 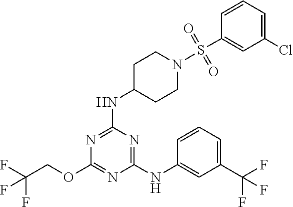 | N-{1-[(3- chlorophenyl) sulfonyl]piperidin- 4-yl}-6-(2,2,2- trifluoroethoxy)- N′-[3-(trifluoro- methyl)phenyl]- 1,3,5-triazine- 2,4-diamine | 612.0 | C₂₃H₂₁ClF₆N₆O₃S |
| 5 | 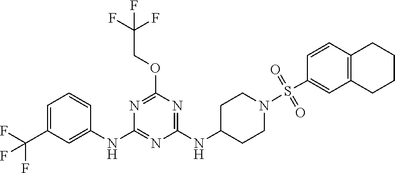 | N-[1-(5,6,7,8- tetrahydro- napthalen-2- ylsulfonyl)- piperidin-4-yl]- 6-(2,2,2- trifluoroethoxy)- N′-[3-(trifluoro- methyl)phenyl]- 1,3,5-triazine- 2,4-diamine | 631.6 | C₂₇H₂₈F₆N₆O₃S |
| 6 | 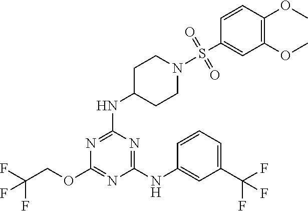 | N-{1-[(3,4- dimethoxy- phenyl)sulfonyl] piperidin-4-yl}-6- (2,2,2-trifluoro- ethoxy)-N′-[3- (trifluoromethyl) phenyl]-1,3,5- triazine-2,4- diamine | 637.6 | C₂₅H₂₆F₆N₆O₅S |
| 7 | 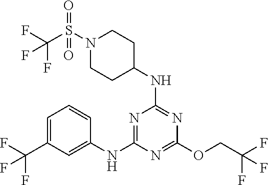 | 6-(2,2,2-trifluoro- ethoxy)-N-[3- (trifluoromethyl)- phenyl]-N′-{1- [(trifluoromethyl) sulfonyl]piperidin- 4-yl}-1,3,5-triazine- 2,4-diamine | 569.4 | C₁₈H₁₇F₉N₆O₃S |
| 8 | 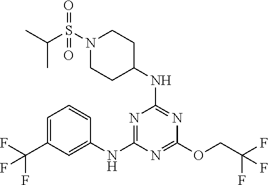 | N-[1-(propan- 2-ylsulfonyl) piperidin-4-yl]- 6-(2,2,2-trifluoro- ethoxy)-N′-[3- (trifluoromethyl) phenyl]-1,3,5- triazine-2,4- diamine | 543.5 | C₂₀H₂₄F₆N₆O₃S |
| 9 | 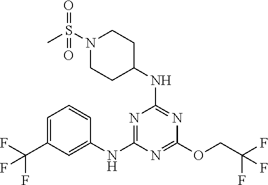 | N-[1-(methyl- sulfonyl)piperidin- 4-yl]-6-(2,2,2- trifluoroethoxy)- N′-[3-(trifluoro- methyl)phenyl]- 1,3,5-triazine-2,4- diamine | 515.5 | C₁₈H₂₀F₆N₆O₃S |
| 10 | 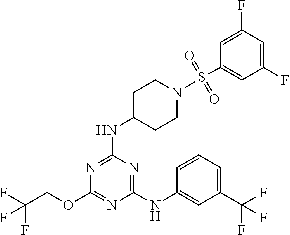 | N-{1-[(3,5- difluorophenyl) sulfonyl]- piperidin-4-yl}-6- (2,2,2-trifluoro- ethoxy)-N′-[3- (trifluoromethyl) phenyl]-1,3,5- triazine-2,4- diamine | 613.5 | C₂₃H₂₀F₈N₆O₃S |
| 11 | 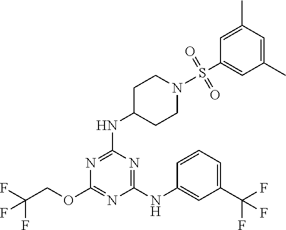 | N-{1-[(3,5- dimethylphenyl) sulfonyl]piperidin- 4-yl}-6-(2,2,2- trifluoroethoxy)- N′-[3-(trifluoro- methyl)phenyl]- 1,3,5-triazine- 2,4-diamine | 605.6 | C₂₅H₂₆F₆N₆O₃S |
| 12 | 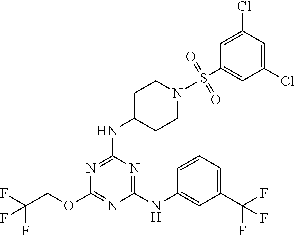 | N-{1-[(3,5- dichlorophenyl) sulfonyl]piperidin- 4-yl}-6-(2,2,2- trifluoroethoxy)- N′-[3-(trifluoro- methyl)phenyl]- 1,3,5-triazine- 2,4-diamine | 646.4 | C₂₃H₂₀Cl₂F₆N₆O₃S |
| 13 | 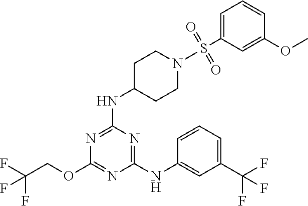 | N-{1-[(3-methoxy- phenyl)sulfonyl] piperidin-4-yl}- 6-(2,2,2-trifluoro- ethoxy)-N′-[3- (trifluoromethyl) phenyl]-1,3,5- triazine-2,4- diamine | 607.6 | C₂₄H₂₄F₆N₆O₄S |
| 14 | 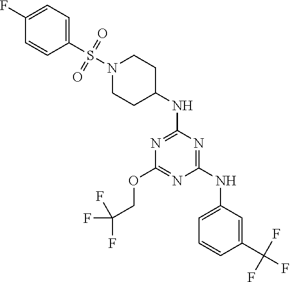 | N-{1-[(3-fluoro- phenyl)sulfonyl] piperidin-4- yl}-6-(2,2,2- trifluoroethoxy)- N′-[3-(trifluoro- methyl)phenyl]- 1,3,5-triazine- 2,4-diamine | 594.5 | C₂₃H₂₁F₇N₆O₃S |
| 15 | 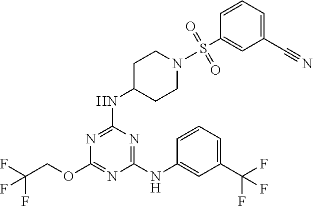 | 3-[(4-{[4-(2,2,2- trifluoroethoxy)- 6-{[3-(trifluoro- methyl)phenyl] amino}-1,3,5- triazin-2-yl]amino} piperidin-1-yl) sulfonyl] benzonitrile | 602.5 | C₂₄H₂₁F₆N₇O₃S |
| 16 | 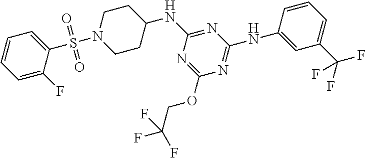 | N-{1-[(2-fluoro- phenyl)sulfonyl] piperidin-4-yl}-6- (2,2,2-trifluoro- ethoxy)-N′-[3- (trifluoromethyl) phenyl]-1,3,5- triazine-2,4- diamine | 594.5 | C₂₃H₂₁F₇N₆O₃S |
| 17 | 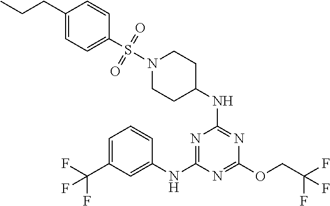 | N-{1-[(4-propyl- phenyl)sulfonyl] piperidin-4-yl}- 6-(2,2,2-trifluoro- ethoxy)-N′-[3- (trifluoromethyl) phenyl]-1,3,5- triazine-2,4- diamine | 619.6 | C₂₆H₂₈F₆N₆O₃S |
| 18 | 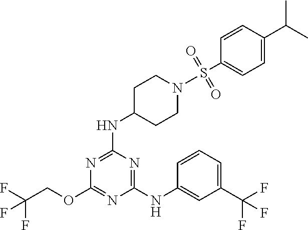 | N-(1-{[4-propan- 2-yl)phenyl] sulfonyl}piperidin- 4-yl)-6-(2,2,2- trifluoroethoxy)- N′-[3-(trifluoro- methyl)phenyl]- 1,3,5-triazine- 2,4-diamine | 619.6 | C₂₆H₂₈F₆N₆O₃S |
| 19 | 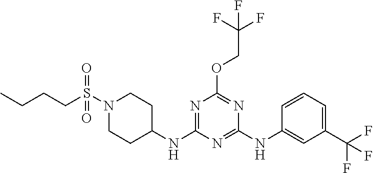 | N-[1-(butyl- sulfonyl)piperidin- 4-yl]-6-(2,2,2- trifluoroethoxy)- N′-[3-(trifluoro- methyl)phenyl]- 1,3,5-triazine- 2,4-diamine | 557.5 | C₂₁H₂₆F₆N₆O₃S |
| 20 | 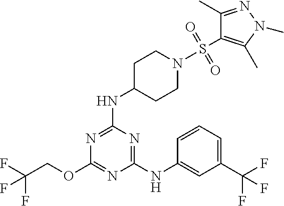 | 6-(2,2,2-trifluoro- ethoxy)-N-[3- (trifluoromethyl)- phenyl]-N′-{1- [(1,3,5-trimethyl- 1H-pyrazol-4- yl)sulfonyl] piperidin-4-yl}- 1,3,5-triazine- 2,4-diamine | 609.6 | C₂₃H₂₆F₆N₈O₃S |
| 21 | 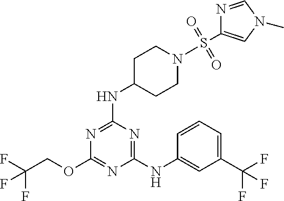 | N-{1-[(1-methyl- 1H-imidazol-4-yl) sulfonyl]piperidin- 4-yl}-6-(2,2,2- trifluoroethoxy)- N′-[3-(trifluoro- methyl)phenyl]- 1,3,5-triazine- 2,4-diamine | 581.5 | C₂₁H₂₂F₆N₈O₃S |
| 22 | 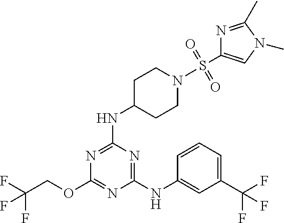 | N-{1-[(1,2- dimethyl-1H- imidazol-4-yl) sulfonyl]- piperidin-4-yl}- 6-(2,2,2-trifluoro- ethoxy)-N′-[3- (trifluoromethyl) phenyl]- 1,3,5-triazine- 2,4-diamine | 595.5 | C₂₂H₂₄F₆N₈O₃S |
| 23 | 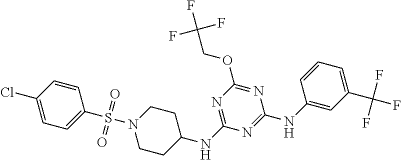 | N-{1-[(4-chloro- phenyl)sulfonyl] piperidin-4-yl}-6- (2,2,2-trifluoro- ethoxy)-N′- [3-(trifluoro- methyl)phenyl]- 1,3,5-triazine- 2,4-diamine | 611.0 | C₂₃H₂₁ClF₆N₆O₃S |
| 24 | 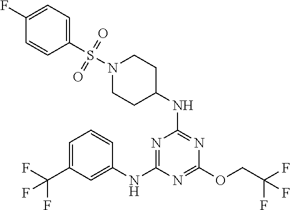 | N-{1-[(4-fluoro- phenyl)sulfonyl] piperidin-4-yl}- 6-(2,2,2-trifluoro- ethoxy)-N′-[3- (trifluoromethyl) phenyl]-1,3,5- triazine- 2,4-diamine | 595.5 | C₂₃H₂₁F₇N₆O₃S |
| 25 | 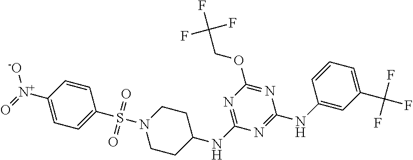 | N2-(1-(4-nitro- phenylsulfonyl) piperidin-4-yl)- 6-(2,2,2-trifluoro- ethoxy)-N4-(3- (trifluoromethyl) phenyl)-1,3,5- triazine-2,4- diamine | 621.5 | C₂₃H₂₁F₆N₇O₅S |
| 26 | 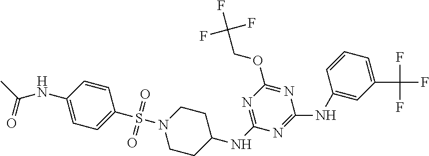 | N-[4-({4-[(4- (2,2,2-trifluoro- ethoxy)-6-{[3- (trifluoromethyl) phenyl]amino}- 1,3,5-triazin- 2-yl)amino] piperidin- 1-yl}sulfonyl) phenyl] acetamide | 633.6 | C₂₅H₂₅F₆N₇O₄S |
| 27 | 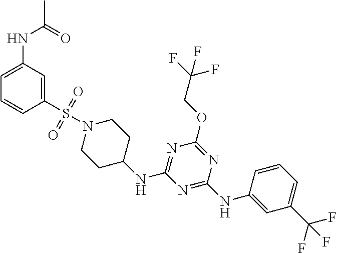 | N-[3-({4-[(4- (2,2,2-trifluoro- ethoxy)-6-{[3- (trifluoromethyl) phenyl]amino}- 1,3,5-triazin-2-yl) amino]piperidin-1- yl}sulfonyl) phenyl]acetamide | 633.6 | C₂₅H₂₅F₆N₇O₄S |
| 28 | 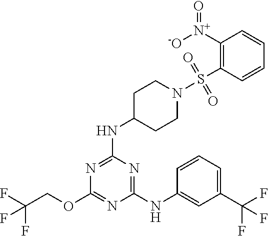 | N-{1-[(2-nitro- phenyl)sulfonyl] piperidin-4-yl}- 6-(2,2,2-trifluoro- ethoxy)-N′-[3- (trifluoromethyl) phenyl]- 1,3,5-triazine- 2,4-diamine | 622.5 | C₂₃H₂₁F₆N₇O₅S |
| 29 | 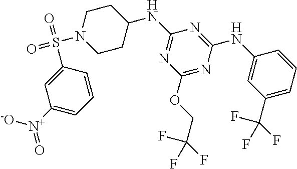 | N2-(1-(3-nitro- phenylsulfonyl) piperidin-4-yl)- 6-(2,2,2-trifluoro- ethoxy)-N4-(3- (trifluoromethyl) phenyl)-1,3,5- triazine-2,4-diamine | 621.5 | C₂₃H₂₁F₆N₇O₅S |
| 30 | 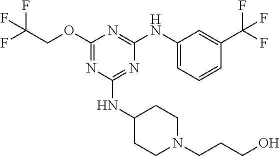 | N-[1-(propyl- sulfonyl)piperidin- 4-yl]-6-(2,2,2- trifluoroethoxy)- N′-[3-(trifluoro- methyl)phenyl]- 1,3,5-triazine- 2,4-diamine | 542.5 | C₂₀H₂₄F₆N₆O₃S |
| 31 | 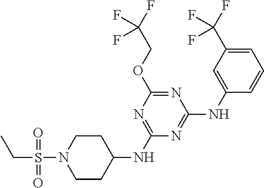 | N-[1-(ethyl- sulfonyl)piperidin- 4-yl]-6-(2,2,2- trifluoroethoxy)- N′-[3-(trifluoro- methyl)phenyl]- 1,3,5-triazine- 2,4-diamine | 528.5 | C₁₉H₂₂F₆N₆O₃S |
| 32 | 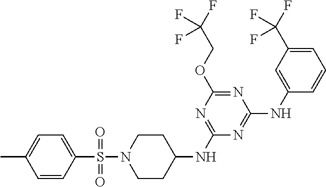 | N-{1-[(4-methyl- phenyl)sulfonyl] piperidin-4-yl}- 6-(2,2,2-trifluoro- ethoxy)-N′-[3- (trifluoromethyl) phenyl]-1,3,5- triazine-2,4- diamine | 590.6 | C₂₄H₂₄F₆N₆O₃S |
| 33 | 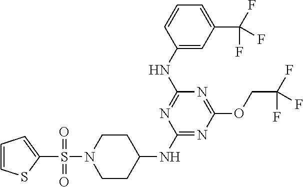 | N-[1-(thien-2- ylsulfonyl) piperidin-4-yl]- 6-(2,2,2- trifluoroethoxy)- N′-[3-(trifluoro- methyl)phenyl]- 1,3,5-triazine- 2,4-diamine | 582.6 | C₂₁H₂₀F₆N₆O₃S₂ |
| 34 | 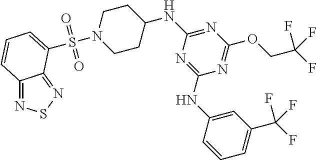 | N-[1-(2,1,3- benzo- thiadiazol- 4-ylsulfonyl) piperidin-4- yl]-6-(2,2,2- trifluoroethoxy)- N′-[3-(trifluoro- methyl)phenyl]- 1,3,5-triazine- 2,4-diamine | 634.6 | C₂₃H₂₀F₆N₈O₃S₂ |
| 35 | 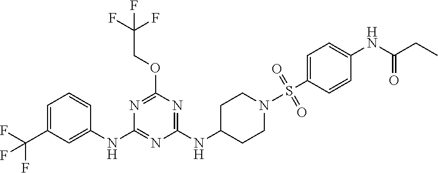 | N-{4-[(4- {[4-(2,2,2- trifluoroethoxy)- 6-{[3-(trifluoro- methyl)phenyl] amino}-1,3,5- triazin-2-yl] amino}-piperidin- 1-yl)sulfonyl] phenyl} propanamide | 648.6 | C₂₆H₂₇F₆N₇O₄S |
| 36 | 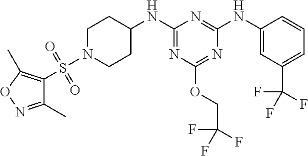 | N-{1-[(3,5- dimethyl- isoxazol-4-yl) sulfonyl]piperidin- 4-yl}-6-(2,2,2- trifluoroethoxy)- N′-[3-(trifluoro- methyl)phenyl]- 1,3,5-triazine- 2,4-diamine | 595.5 | C₂₂H₂₃F₆N₇O₄S |
| 37 | 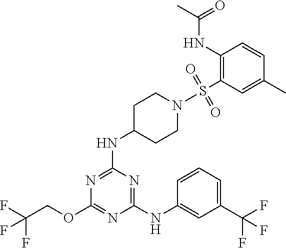 | N-{4-methyl- 2-[(4-{[4- (2,2,2-trifluoro- ethoxy)-6- {[3-(trifluoro- methyl)phenyl] amino}-1,3,5- triazin-2-yl] amino}piperidin- 1-yl)sulfonyl] phenyl}acetamide | 648.6 | C₂₆H₂₇F₆N₇O₄S |
| 38 | 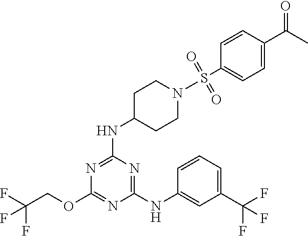 | 1-{4-[(4-{[4- (2,2,2-trifluoro- ethoxy)-6-{[3- (trifluoromethyl) phenyl]amino}- 1,3,5-triazin-2-yl] amino}piperidin- 1-yl)sulfonyl] phenyl}ethanone | 619.6 | C₂₅H₂₄F₆N₆O₄S |
| 39 | 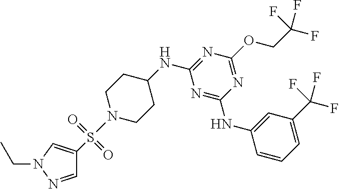 | N-{1-[(1-ethyl- 1H-pyrazol- 4-yl)sulfonyl] piperidin-4- yl}-6-(2,2,2- trifluoroethoxy)- N′-[3-(trifluoro- methyl)phenyl]- 1,3,5-triazine- 2,4-diamine | 594.5 | C22H24F6N8O3S |

### Procedures and Analytical Data for Table 33.

Entries 1 to 25 and entries 28 to 39 are analogs from Library 33a. Entries 26 and 27 are from Library 33b.

### 1. N-[1-(benzylsulfonyl)piperidin-4-yl]-6-(2,2,2-trifluoroethoxy)-N′-[3-(trifluoromethyl)phenyl]-1,3,5-triazine-2,4-diamine

LCMS: M+1=590.5; 1H NMR (DMSO-d6, 90° C., ppm): I=1.19 s (2H), 1.68 m (2H), 1.92 m (2H), 3.62 d (2H), 3.95 s (1H), 4.36 s (2H), 4.99 m (2H), 7.44 m (8H), 8.01 d (2H), 9.95 s 1H).

### 2. N-{1-[(3,4-difluorophenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=612.5; 1H NMR (DMSO-d6, 90° C., ppm): I=1.62 m (2H), 1.92 m (2H), 2.60 m (2H), 3.62 m (2H), 4.85 s (1H), 4.90 m (2H), 7.78 m (8H), 9.60 s (1H).

### 3. N-{1-[(3,5-dimethyl-1H-pyrazol-4-yl)sulfonyl]piperidin-4-yl}-6-(2,2,2-trifluoroethoxy)-N′-[3-(trifluoromethyl)phenyl]-1,3,5-triazine-2,4-diamine

1H NMR (DMSO-d6, 90° C., ppm): d=1.53-1.70 m (2H, CH2), 1.88-2.01 m (2H, CH2), 2.33 s (6H, 2CH3), 2.61 t (2H, CH2), 3.56 d (2H, CH2), 3.85 m (1H, CH), 4.92 q (2H, CH2), 7.30 d (1H, Ar), 7.48 t (2H, Ar), 7.82 s br. (1H, Ar), 8.20 m br. (1H, NH), 9.59 s br. (1H, NH), 12.78 s br. (1H, NH). LC-MS [M+1]: calc'd: 595.5; obs'd: 595.2.

### 4. N-{1-[(3-chlorophenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=610.9; 1H NMR (DMSO-d6, 90° C., ppm): 1=1.63 m (2H), 1.95 m (2H), 2.65 m (2H), 3.59 d (2H), 3.89 m (1H), 4.95 m (2H), 7.71 m (8H), 9.62 s (1H).

### 5. N-[1-(5,6,7,8-tetrahydronaphthalen-2-ylsulfonyl)piperidin-4-yl]-6-(2,2,2-trifluoroethoxy)-N′-[3-(trifluoromethyl)phenyl]-1,3,5-triazine-2,4-diamine

LCMS: M+1=630.6; 1H NMR (DMSO-d6, 90° C., ppm): 1=1.59 m (2H), 1.81 m (2H), 1.92 m (2H), 2.89 m (4H), 3.61 m (2H), 3.81 m (1H), 4.99 m (2H), 7.98 m (8H), 9.51 s (1H).

### 6. N-{1-[(3,4-dimethoxyphenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=636.5; 1H NMR (DMSO-d6, 90° C., ppm): 1=1.68 m (2H), 1.96 m (2H), 2.71 m (2H), 3.63 m (2H), 3.82 m (7H), 4.99 m (2H), 7.28 m (5H), 7.43 t (1H), 7.88 d (1H), 8.16 s (1H), 9.46 s (1H).

### 7. 6-(2,2,2-trifluoroethoxy)-N-[3-(trifluoromethyl)phenyl]-N′-{1-[(trifluoromethyl)sulfonyl]piperidin-4-yl}-1,3,5-triazine-2,4-diamine

LCMS: M+1=559 NMR 1H, DMSO, ppm: 1.8 m (2H, CH); 2.05 t (2H CH); 3.28 m (2H, CH); 3.88 t (2H, CH); 4.05 m (1H, CH); 5.0 m (2H, CH); 7.38 d (1H, CH); 7.58 m (2H, CH); 7.88 d (1H, CH); 8.1 s (1H, NH); 9.6 s (1H, NH)

### 8. N-[1-(propan-2-ylsulfonyl)piperidin-4-yl]-6-(2,2,2-trifluoroethoxy)-N′-[3-(trifluoromethyl)phenyl]-1,3,5-triazine-2,4-diamine

LCMS: M+1=543 NMR 1H, DMSO-d6 δ, ppm: 1.25 d (6H); 1.62 m (2H); 1.95 m (2H); 3.08 m (2H); 3.2 m (1H); 3.72 bd (2H), 4.01 m (1H); 4.95 m (2H); 7.32 d (1H); 7.54 t (2H); 7.92 bs (1H); 8.23 bs (1H); 9.63 bs (1H).)

### 9. N-[1-(methylsulfonyl)piperidin-4-yl]-6-(2,2,2-trifluoroethoxy)-N′-[3-(trifluoromethyl)phenyl]-1,3,5-triazine-2,4-diamine

LCMS: M+1=515 NMR 1H, DMSO-d6 δ, ppm: 1.67 m (2H); 1.98 m (2H); 2.50 s (3H); 2.85 m (2H); 3.62 m (2H) 3.95 bs (1H); 4.95 m (2H); 7.35 d (1H); 7.53 t (2H); 7.92 bs (1H); 8.18 bs

### 10. N-{1-[(3,5-difluorophenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=613.4 NMR 1H, DMSO-d6 δ, ppm: 1.63 m (2H); 1.97 m (2H); 2.70 m (2H); 3.68 m (2H); 3.90 m (1H); 4.92 m (2H); 7.28 d (1H); 7.50 m (5H); 7.85 bs (1H); 8.20 bs (1H); 9.57 bs (1H).

### 11. N-{1-[(3,5-dimethylphenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=605.6 NMR 1H, DMSO-d6 δ, ppm: 1.65 m (2H); 1.95 m (2H); 2.40 (6H); 2.57 m (2H); 3.63 m (2H); 3.82 bm (1H); 4.91 m (2H); 7.34 m (4H); 7.47 t (2H); 8.02 bm (2H); 9.54 bs (1H).

### 12. N-{1-[(3,5-dichlorophenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=645.6 NMR 1H, DMSO-d6 δ, ppm: 1.65 m (2H); 1.95 m (2H); 2.72 t (2H); 3.69 m (2H); 3.90 bm (1H); 4.92 m (2H); 7.29 d (1H); 7.49 t (2H); 7.72-7.92 bm (3H); 8.20 bs (1H); 9.57 bs (1H).

### 13. N-{1-[(3-methoxyphenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=607.6 NMR 1H, DMSO-d6 δ, ppm: 1.65 m (2H); 1.95 m (2H); 2.57 m (2H); 3.63 m (2H); 3.82 bm (1H); 3.83 s (3H); 4.91 m (2H); 7.20-7.60 m (7H); 7.80-8.20 m (2H); 9.54 bs (1H).

### 14. N-{1-[(3-fluorophenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=595.6 NMR 1H, DMSO-d6 δ, ppm: 1.65 m (2H); 1.95 m (2H); 2.72 t (2H); 3.69 m (2H); 3.90 bm (1H); 4.92 m (2H); 7.29 d (1H); 7.41-8.20 m (8H); 9.57 bs (1H).

### 15. 3-[(4-{[4-(2,2,2-trifluoroethoxy)-6-{[3-(trifluoromethyl)phenyl]amino}-1,3,5-triazin-2-yl]amino}piperidin-1-yl)sulfonyl]benzonitrile

LCMS: M+1=602.6 NMR 1H, DMSO-d6 δ, ppm: 1.65 m (2H); 1.95 m (2H); 2.72 t (2H); 3.69 m (2H); 3.90 bm (1H); 4.92 m (2H); 7.29 d (1H); 7.49 t (2H); 7.85 t (2H); 8.05-8.15 m (4H); 9.57 bs (1H).

### 16. N-{1-[(2-fluorophenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=595.6 NMR 1H, DMSO-d6 δ, ppm: 1.65 m (2H); 1.95 m (2H); 2.72 t (2H); 3.69 m (2H); 3.90 bm (1H); 4.92 m (2H); 7.29 d (1H); 7.49 m (4H); 7.82 m (3H); 8.20 bs (1H); 9.57 bs (1H).

### 17. N-{1-[(4-propylphenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=619.6 NMR 1H, DMSO-d6 δ, ppm: 0.95 t (3H); 1.65 m (4H); 1.95 m (2H); 2.62 m (2H); 2.72 t (2H); 3.65 m (2H); 3.85 bm (1H); 4.92 m (2H); 7.29 d (1H); 7.49 m (4H); 7.67 d (2H); 7.80-8.15 m (2H); 9.57 bs (1H).

### 18. N-(1-{[4-(propan-2-yl)phenyl]sulfonyl}piperidin-4-yl)-6-(2,2,2-trifluoroethoxy)-N′-[3-(trifluoromethyl)phenyl]-1,3,5-triazine-2,4-diamine

LCMS: M+1=619.6 NMR 1H, DMSO-d6 δ, ppm: 1.28 d (6H); 1.65 m (2H); 1.95 m (2H); 2.62 m (2H); 3.00 m (1H); 3.65 m (2H); 3.85 bm (1H); 4.92 m (2H); 7.29 d (1H); 7.49 m (4H); 7.67 d (2H); 7.80-8.15 m (2H); 9.57 bs (1H).

### 19. N-[1-(butylsulfonyl)piperidin-4-yl]-6-(2,2,2-trifluoroethoxy)-N′-[3-(trifluoromethyl)phenyl]-1,3,5-triazine-2,4-diamine

LCMS: M+1=557.6 NMR 1H, DMSO-d6 δ, ppm: 0.95 t (3H); 1.40-1.80 m (6H); 1.95 m (2H); 2.90-3.05 m (4H); 3.65 m (2H); 3.99 bm (1H); 4.92 m (2H); 7.29 d (1H); 7.49 m (2H); 7.80-8.15 m (2H); 9.57 bs (1H).

### 20. 6-(2,2,2-trifluoroethoxy)-N-[3-(trifluoromethyl)phenyl]-N′-{1-[(1,3,5-trimethyl-1H-pyrazol-4-yl)sulfonyl]piperidin-4-yl}-1,3,5-triazine-2,4-diamine

LCMS: M+1=605.6 NMR 1H, DMSO-d6 δ, ppm: 1.65 m (2H); 1.95 m (2H); 2.28 (3H); 2.44 s (3H); 2.57 t (2H); 3.60 m (2H); 3.72 s (3H); 3.82 bm (1H); 4.91 m (2H); 7.29 d (1H); 7.49 m (2H); 7.80-8.15 m (2H); 9.57 bs (1H).

### 21. N-{1-[(1-methyl-1H-imidazol-4-yl)sulfonyl]piperidin-4-yl}-6-(2,2,2-trifluoroethoxy)-N′-[3-(trifluoromethyl)phenyl]-1,3,5-triazine-2,4-diamine

LCMS: M+1=581.6 NMR 1H, DMSO-d6 δ, ppm: 1.60 m (2H); 1.95 m (2H); 2.72 t (2H); 3.58-3.90 m (6H); 4.92 m (2H); 7.29 d (1H); 7.49 m (2H); 7.70 d (2H); 7.80-8.20 m (2H); 9.57 bs (1H)

### 22. N-{1-[(1,2-dimethyl-1H-imidazol-4-yl)sulfonyl]piperidin-4-yl}-6-(2,2,2-trifluoroethoxy)-N′-[3-(trifluoromethyl)phenyl]-1,3,5-triazine-2,4-diamine

LCMS: M+1=595.6 NMR 1H, DMSO-d6 δ, ppm: 1.65 m (2H); 1.95 m (2H); 2.35 (3H); 2.72 t (2H); 3.65 m (5H); 3.82 bm (1H); 4.91 m (2H); 7.25-7.50 m (3H); 7.60 s (1H); 7.85 bs (1H); 8.15 bs (1H); 9.57 bs (1H).

### 23. N-{1-[(4-chlorophenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=611.1 NMR 1H, DMSO-d6 δ, ppm: 1.65 m (2H); 1.95 m (2H); 2.65 (2H); 3.69 m (2H); 3.90 bm (1H); 4.92 m (2H); 7.29 d (1H); 7.49 t (2H); 7.70 d (2H); 7.83 d (2H); 8.18 bs (1H); 9.57 bs (1H).

### 24. N-{1-[(4-fluorophenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=595.6 NMR 1H, DMSO-d6 δ, ppm: 1.65 m (2H); 1.95 m (2H); 2.65 (2H); 3.69 m (2H); 3.90 bm (1H); 4.92 m (2H); 7.29 d (1H); 7.49 t (2H); 7.70 d (2H); 7.83 d (2H); 8.18 bs (1H); 9.57 bs (1H).

### 25. N-2-(1-(4-nitrophenylsulfonyl)piperidin-4-yl)-6-(2,2,2-trifluoroethoxy)-N-4-(3-(trifluoromethyl)phenyl)-1,3,5-triazine-2,4-diamine

LCMS: M+1=622.6 NMR 1H, DMSO-d6 δ, ppm: 1.65 m (2H); 1.95 m (2H); 2.69 (2H); 3.69 m (2H); 3.90 bm (1H); 4.92 m (2H); 7.29 d (1H); 7.49 t (2H); 7.84 bs (1H); 8.05 d (2H); 8.18 bs (1H); 8.45 d (2H); 9.57 bs (1H).

### 26. N-[4-({4-[(4-(2,2,2-trifluoroethoxy)-6-{[3-(trifluoromethyl)phenyl]amino}-1,3,5-triazin-2-yl)amino]piperidin-1-yl}sulfonyl)phenyl]acetamide

LCMS: M+1=634.6 NMR 1H, DMSO-d6 δ, ppm: 1.65 m (2H); 1.95 m (2H); 2.12 d (3H); 2.65 t (2H); 3.69 m (2H); 3.90 bm (1H); 4.92 m (2H); 7.29 d (1H); 7.49 t (2H); 7.70 d (2H); 7.83 d (3H); 8.18 bs (1H); 9.57 bs (1H); 10.06 bs (1H)

### 27. N-[3-({4-[(4-(2,2,2-trifluoroethoxy)-6-{[3-(trifluoromethyl)phenyl]amino}-1,3,5-triazin-2-yl)amino]piperidin-1-yl}sulfonyl)phenyl]acetamide

LCMS: M+1=634.6 NMR 1H, DMSO-d6 δ, ppm: 0.97 t (1H); 1.65 m (2H); 1.95 m (2H); 2.10 s (3H); 2.60 m (2H); 3.62 m (2H); 3.85 bm (1H); 4.92 m (2H); 7.29 d (1H); 7.37-7.57 m (4H); 7.85 d (2H); 8.05-8.20 m (2H); 9.57 bs (1H); 9.96 bs (1H).

### 28. N-{1-[(2-nitrophenyl)sulfonyl]piperidin-4-yl}-6-(2,2,2-trifluoroethoxy)-N′-[3-(trifluoromethyl)phenyl]-1,3,5-triazine-2,4-diamine

LCMS: M+1=622.6 NMR 1H, DMSO-d6 δ, ppm: 1.65 m (2H); 1.95 m (2H); 2.72 t (2H); 3.72 m (2H); 3.90 bm (1H); 4.92 m (2H); 7.30 d (1H); 7.50 t (2H); 7.90 m (4H); 8.05 d (1H); 8.20 bs (1H); 9.57 bs (1H).

### 29. N-2-(1-(3-nitrophenylsulfonyl)piperidin-4-yl)-6-(2,2,2-trifluoroethoxy)-N-4-(3-(trifluoromethyl)phenyl)-1,3,5-triazine-2,4-diamine

LCMS: M+1=622.6 NMR 1H, DMSO-d6 δ, ppm: 1.65 m (2H); 1.95 m (2H); 2.72 t (2H); 3.72 m (2H); 3.90 bm (1H); 4.92 m (2H); 7.30 d (1H); 7.50 t (2H); 7.80 bm (1H); 7.95 t (1H); 8.20 m (2H); 8.40 s (1H); 8.50 d (1H); 9.57 bs (1H).

### 30. N-[1-(propylsulfonyl)piperidin-4-yl]-6-(2,2,2-trifluoroethoxy)-N′-[3-(trifluoromethyl)phenyl]-1,3,5-triazine-2,4-diamine

LCMS: M+1=543.6 NMR 1H, DMSO-d6 δ, ppm: 1.00 t (3H); 1.66 m (4H); 1.95 d (2H); 2.72 t (2H); 2.95 m (2H); 3.64 d (2H); 3.99 m (1H); 4.92 m (2H); 7.29 d (1H); 7.49 m (2H); 7.70 d (2H); 7.80-8.20 m (2H); 9.57 bs (1H);

### 31. N-[1-(ethylsulfonyl)piperidin-4-yl]-6-(2,2,2-trifluoroethoxy)-N′-[3-(trifluoromethyl)phenyl]-1,3,5-triazine-2,4-diamine

LCMS: M+1=529.6 NMR 1H, DMSO-d6 δ, ppm: 1.25 t (3H); 1.62 m (2H); 1.95 d (2H); 2.95 m (2H); 3.64 d (2H); 3.99 m (1H); 4.92 m (2H); 7.29 d (1H); 7.49 m (2H); 7.70 d (2H); 7.80-8.20 m (2H); 9.57 bs (1H);

### 32. N-{1-[(4-methylphenyl)sulfonyl]piperidin-4-yl}-6-(2,2,2-trifluoroethoxy)-N′-[3-(trifluoromethyl)phenyl]-1,3,5-triazine-2,4-diamine

NMR 1H, DMSO-d6 δ, ppm: 1.65 m (2H); 1.95 m (2H); 2.40 s (3H); 2.65 (2H); 3.69 m (2H); 3.90 bm (1H); 4.92 m (2H); 7.20-7.60 m (8H); 7.70 m (1H); 9.57 bs (1H).

### 33. N-[1-(thien-2-ylsulfonyl)piperidin-4-yl]-6-(2,2,2-trifluoroethoxy)-N′-[3-(trifluoromethyl)phenyl]-1,3,5-triazine-2,4-diamine

LCMS: M+1=583.6 NMR 1H, DMSO-d6 δ, ppm: 1.65 m (2H); 1.95 m (2H); 2.72 t (2H); 3.72 m (2H); 3.90 bm (1H); 4.92 m (2H); 7.20-7.60 m (9H); 7.70-8.01 m (3H); 8.20 bs (1H); 9.57 bs (1H).

### 34. N-[1-(2,1,3-benzothiadiazol-4-ylsulfonyl)piperidin-4-yl]-6-(2,2,2-trifluoroethoxy)-N′-[3-(trifluoromethyl)phenyl]-1,3,5-triazine-2,4-diamine

LCMS: M+1=635.6 NMR 1H, DMSO-d6 δ, ppm: 1.65 m (2H); 1.95 m (2H); 2.72 t (2H); 3.72 m (2H); 3.90 bm (1H); 4.92 m (2H); 7.20-7.50 m (3H); 7.75 m (2H); 8.15 bs (1H); 8.20 d (1H); 8.40 d (1H); 9.57 bs (1H).

### 35. N-{4-[(4-{[4-(2,2,2-trifluoroethoxy)-6-{[3-(trifluoromethyl)phenyl]amino}-1,3,5-triazin-2-yl]amino}piperidin-1-yl)sulfonyl]phenyl}propanamide

LCMS: M+1=648.6 NMR 1H, DMSO-d6 δ d, ppm: 1.14 t (3H); 1.65 m (2H); 1.95 m (2H); 2.38 m (2H); 2.65 m (2H); 3.69 m (2H); 3.90 bm (1H); 4.92 m (2H); 7.28 d (1H); 7.40-8.20 m (8H); 9.57 bs (1H), 10.00 s (1H).

### 36. N-{1-[(3,5-dimethylisoxazol-4-yl)sulfonyl]piperidin-4-yl}-6-(2,2,2-trifluoroethoxy)-N′-[3-(trifluoromethyl)phenyl]-1,3,5-triazine-2,4-diamine

LCMS: M+1=596.6 NMR 1H, DMSO-d6 δ, ppm: 1.65 m (2H); 1.95 m (2H); 2.35 (3H); 2.63 s (3H); 2.82 t (2H); 3.60 m (2H); 3.72 s (3H); 3.82 bm (1H); 4.91 m (2H); 7.29 d (1H); 7.49 m (2H); 7.80-8.15 m (2H); 9.57 bs (1H).

### 37. N-{4-methyl-2-[(4-{[4-(2,2,2-trifluoroethoxy)-6-{[3-(trifluoromethyl)phenyl]amino}-1,3,5-triazin-2-yl]amino}piperidin-1-yl)sulfonyl]phenyl}acetamide

LCMS: M+1=648.6 NMR 1H, DMSO-d6 δ, ppm: 1.14 t (3H); 1.65 m (2H); 1.95 m (2H); 2.08 s (3H); 2.52 d (3H); 2.65 m (2H); 3.69 m (2H); 3.90 bm (1H); 4.92 m (2H); 7.30 t (2H); 7.50 m (2H); 7.70-8.20 m (4H); 9.57 bs (1H), 9.90 s (1H).

### 38. 1-{4-[(4-{[4-(2,2,2-trifluoroethoxy)-6-{[3-(trifluoromethyl)phenyl]amino}-1,3,5-triazin-2-yl]amino}piperidin-1-yl)sulfonyl]phenyl}ethanone

LCMS: M+1=619.6 NMR 1H, DMSO-d6 δ, ppm: 1.65 m (2H); 1.95 m (2H); 2.63 s (3H); 2.65 (2H); 3.69 m (2H); 3.90 bm (1H); 4.92 m (2H); 7.20-7.50 m (3H); 7.90 m (3H); 8.15 d (3H); 9.57 bs (1H).

### 39. N-{1-[(1-ethyl-1H-pyrazol-4-yl)sulfonyl]piperidin-4-yl}-6-(2,2,2-trifluoroethoxy)-N′-[3-(trifluoromethyl)phenyl]-1,3,5-triazine-2,4-diamine

LCMS: M+1=595.6 NMR 1H, DMSO-d6 δ, ppm: 1.45 t (3H); 1.65 m (2H); 1.95 m (2H); 2.72 t (2H); 3.72 m (2H); 3.90 bm (1H); 4.22 m (2H); 4.92 m (2H); 7.25-8.25 m (7H); 9.57 bs (1H).

### Generic Synthesis of Intermediates and Final Compounds for Table 34

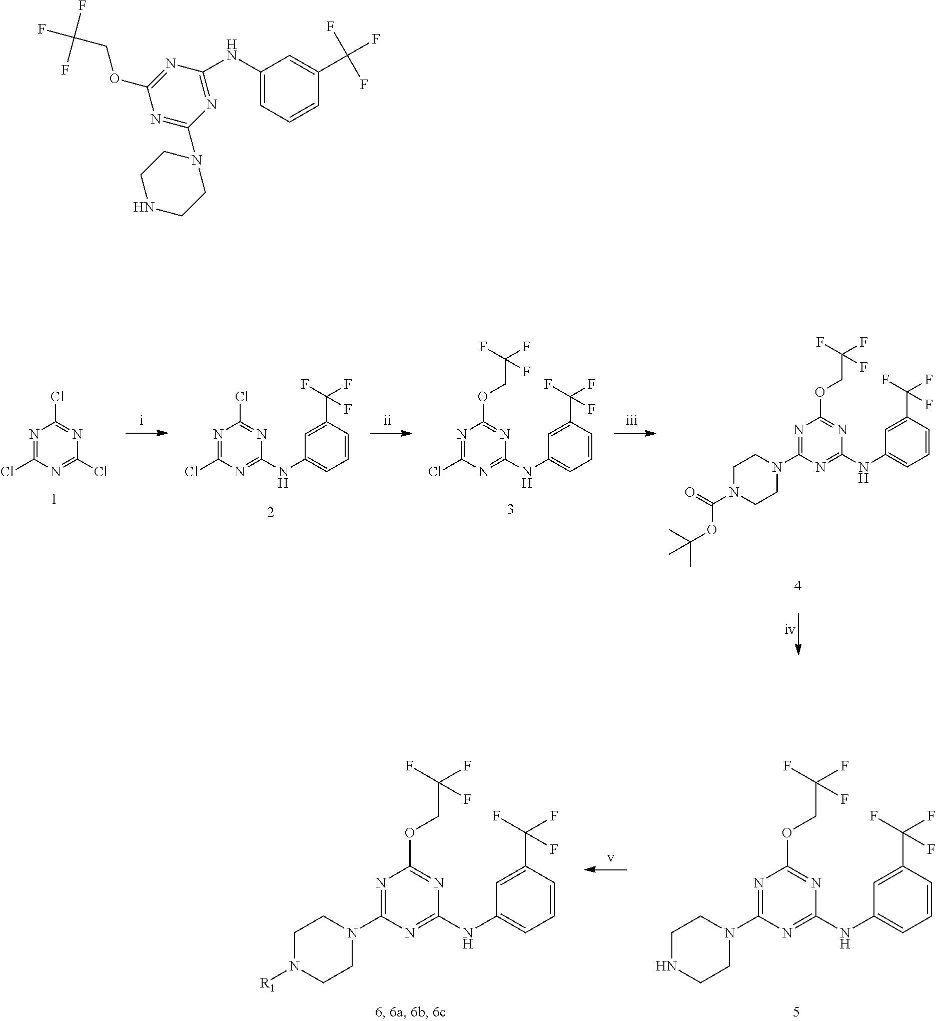

Intermediate 2, Steps i: In a 1-L flask, 50.0 g (0.27 mol) of cyanuric chloride 1 and 24.4 g of sodium acetate were dissolved in 500 mL of dry dioxane. The mixture was cooled to 0° C., and 43.8 g (0.27 mol) of 3-(trifluoromethyl)aniline in 100 mL of dioxane were added within 30 min under constant stirring. The temperature of the reaction mixture was maintained at 0 C for 2.5 h till the completion of the reaction (TLC control). The solvent was removed in vacuo. The solids were suspended in CHCl₃, and filtered. The filtrate was concentrated in vacuo, and additionally washed with hexane.

Intermediate 3, Steps ii: In a 2-L flask, 39.6 g (0.128 mol) of 2 were dissolved in 1 L of acetonitrile. The solution was cooled to −30° C. A solution of potassium tert-butoxide (14.4 g, 0.128 mol, 1 eq.) in 2,2,2-trifluoro-1-ethanol (64 g, 0.64 mol, 5 eq.) was added dropwise to the cooled solution of 2 during 1.5 h. After complete addition, the reaction mixture was allowed to warm to r.t., and the reaction was allowed to proceed overnight. The solid precipitate was filtered and washed with dry acetonitrile (3×200 mL). The filtrate was concentrated in vacuo to afford a yellowish oil. To this oil, dry hexane (3×80 mL) was added, the mixture was heated to reflux; after 3 min, the hexane layer was decanted. After three washes, the residual solvent was removed by rotary evaporation, followed by lyophilization to afford the product.

Intermediate 4, Step iii: In a representative procedure, 1 eq. of 3, 1.1 eq. of BOC-piperazine, and 1.2 eq. of triethylamine were stirred at reflux in dioxane for 8 h. The solvent was removed under reduced pressure. Crude intermediate 3 was purified by flash chromatography using 5% MeOH in CHCl₃.

Intermediate 5, Step iv: Intermediate 4 was dissolved in dioxane saturated with gaseous HCl (concentration of HCl is ca. 2 M). Generally, a 40-fold excess of HCl per BOC group was used. After completion of the reaction in 2 h, the solvent was removed under reduced pressure. The residue was re-dissolved in a minimal volume of CHCl₃, and diethyl ether was added until the product precipitated. Re-crystallization in the freezer overnight afforded analytically pure intermediate 5 in the form of 3×HCl salt.

General procedure for preparation of 6. Carboxylic acid (1 eqv) was dissolved in 1,4-dioxane, CDI (1 eq) was added and the reaction mixture was stirred at 80° C. for 1.5 hours. Then intermediate 5 (1 eqv) was added, and the reaction mixture was allowed to stir at 80° C. overnight. At this time, the reaction mixture was diluted with water and the resulting precipitate was collected by filtration.

General procedure to preparation of Sulfonamides 6a. Intermediate 5 (1 eqv) was dissolved in 1,4-dioxane, and sulf° Chloride (1.3 eq) and triethylamine (1.5 eq) were added. The reaction mixture was stirred at 80° C. overnight. At this time, the reaction mixture was diluted with water and the resulting precipitate was collected by filtration.

Amines 6b. A solution of intermediate 5 (10 mmol) in 10 ml of isopropanol was treated with alkyl chloride (10 mmol) and DIPEA (22 mmol). This mixture was stirred at reflux for 3 hours, then cooled and concentrated. The residue was treated with water. The precipitate was filtered, washed with water, dried, and purified by column chromatography.

Ureas 6c were obtained according to the following procedure: A solution of 10 mmol of intermediate 5 in 10 ml of dioxane was treated with isocyanate (10 mmol). This mixture was stirred at reflux for 3 hours, then cooled and poured into water. The precipitate was filtered, washed with water, dried, and purified by column chromatography.

TABLE 34

| En | Structure | IUPAC Name | MW | Formula |
|---|---|---|---|---|
| 1 | 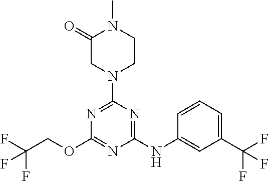 | 1-methyl-4-[4- (2,2,2-trifluoro- ethoxy)-6-{[3- (trifluoromethyl) phenyl]amino}- 1,3,5-triazin-2- yl]piperazin- 2-one | 450.3 | C₁₇H₁₆F₆N₆O₂ |
| 2 | 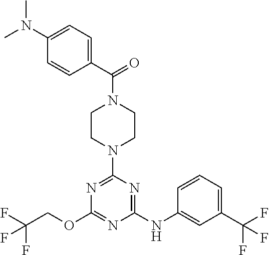 | [4-(dimethyl- amino)phenyl]{4- [4-(2,2,2- trifluoro-ethoxy)- 6-{[3-(trifluoro- methyl)phenyl] amino}-1,3,5- triazin-2-yl] piperazin-1-yl} methanone | 569.5 | C₂₅H₂₅F₆N₇O₄S |
| 3 | 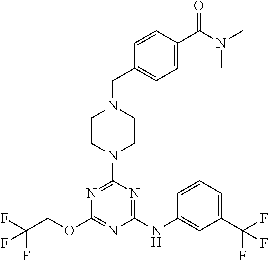 | N,N-dimethyl- 4-((4-(4-(2,2,2- trifluoroethoxy)- 6-(3-(trifluoro- methyl)phenyl- amino)-1,3,5- triazin-2-yl) piperazin-1-yl) methyl)benzamide | 583.5 | C₂₃H₂₁F₆N₇O₅S |
| 4 | 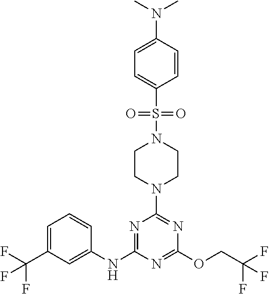 | 4-(4-{[4-(di- methylamino) phenyl]sulfonyl}- piperazin-1-yl)- 6-(2,2,2-trifluoro- ethoxy)-N-[3- (trifluoromethyl) phenyl]-1,3,5- triazin-2-amine | 606.6 | C₂₃H₂₃F₆N₇O₃S |
| 5 | 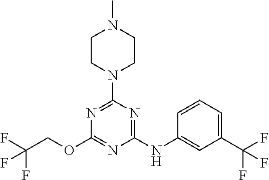 | 4-(4-methyl- piperazin-1- yl)-6-(2,2,2- trifluoro- ethoxy)-N-[3- (trifluoro- methyl)phenyl]- 1,3,5-triazin- 2-amine | 436.4 | C₁₇H₁₈F₆N₆O |
| 6 | 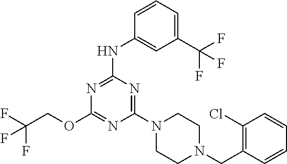 | 4-[4-(2-chloro- benzyl)piperazin- 1-yl]-6-(2,2,2- trifluoroethoxy)- N-[3-(trifluoro- methyl)phenyl]- 1,3,5-triazin-2- amine | 546.9 | C₂₃H₂₁ClF₆N₆O |
| 7 | 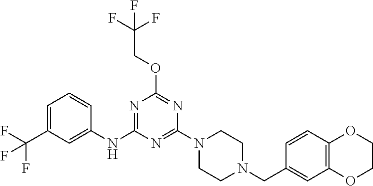 | 4-[4-(2,3-dihydro- 1,4-benzodioxin- 6-ylmethyl) piperazin-1-yl]- 6-(2,2,2-tri- fluoroethoxy)- N-[3-(trifluoro- methyl)phenyl]- 1,3,5-triazin- 2-amine | 570.5 | C₂₅H₂₄F₆N₆O₃ |
| 8 | 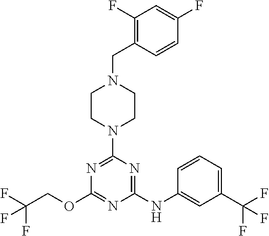 | 4-[4-(2,4-difluoro- benzyl)piperazin- 1-yl]-6-(2,2,2- trifluoroethoxy)- N-[3-(trifluoro- methyl)phenyl]- 1,3,5-triazin-2- amine | 548.4 | C₂₃H₂₀F₈N₆O |
| 9 | 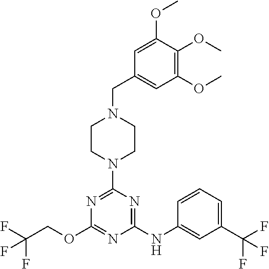 | 4-(2,2,2-trifluoro- ethoxy)-N-[3- (trifluoromethyl) phenyl]-6-[4- (3,4,5-trimethoxy- benzyl)piperazin- 1-yl]-1,3,5-triazin- 2-amine | 602.5 | C₂₆H₂₈F₆N₆O₄ |
| 10 | 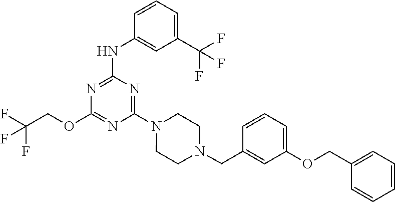 | 4-{4-[3- (benzyloxy) benzyl]-piperazin- 1-yl}-6-(2,2,2- trifluoroethoxy)- N-[3-(trifluoro- methyl)phenyl]- 1,3,5-triazin-2- amine | 618.6 | C₃₀H₂₈F₆N₆O₂ |
| 11 | 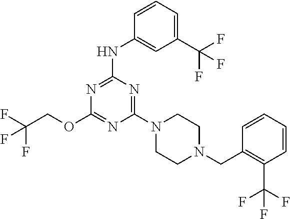 | 4-(2,2,2-trifluoro- ethoxy)-6-{4-[2- (trifluoromethyl) benzyl]piperazin- 1-yl}-N-[3- (trifluoromethyl) phenyl]-1,3,5- triazin-2-amine | 580.5 | C₂₄H₂₁F₉N₆O |
| 12 | 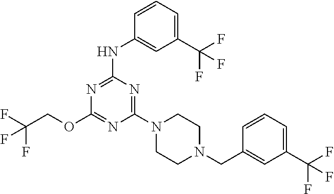 | 4-(2,2,2-trifluoro- ethoxy)-6-{4-[3- (trifluoromethyl) benzyl]piperazin-1- yl}-N-[3-(trifluoro- methyl)phenyl]- 1,3,5-triazin-2- amine | 580.5 | C₂₄H₂₁F₉N₆O |
| 13 | 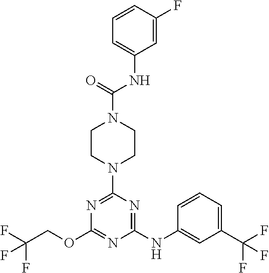 | N-(3-fluoro- phenyl)-4-[4- (2,2,2-trifluoro- ethoxy)-6-{[3-(tri- fluoromethyl) phenyl]amino}- 1,3,5-triazin-2-yl] piperazine-1- carboxamide | 559.4 | C₂₃H₂₀F₇N₇O₂ |
| 14 | 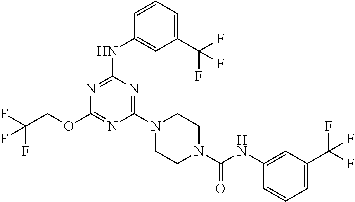 | 4-[4-(2,2,2-tri- fluoroethoxy)-6- {[3-(trifluoro- methyl)-phenyl] amino}-1,3,5- triazin-2-yl]-N-[3- (trifluoromethyl) phenyl]piperazine- 1-carboxamide | 609.4 | C₂₄H₂₀F₉N₇O₂ |
| 15 | 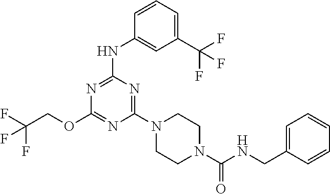 | N-benzyl-4-[4- (2,2,2-trifluoro- ethoxy)-6-{[3- (trifluoromethyl) phenyl]amino}- 1,3,5-triazin-2- yl]piperazine-1- carboxamide | 555.5 | C₂₄H₂₃F₆N₇O₂ |
| 16 | 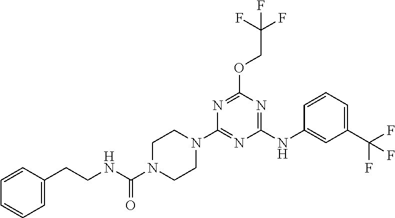 | N-(2-phenylethyl)- 4-[4-(2,2,2-tri- fluoroethoxy)- 6-{[3-(trifluoro- methyl)phenyl] amino}-1,3,5- triazin-2-yl] piperazine-1- carboxamide | 569.5 | C₂₅H₂₅F₆N₇O₂ |
| 17 | 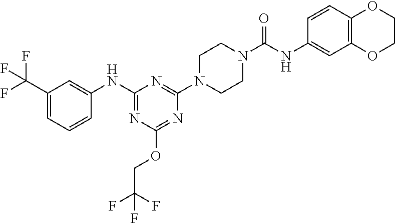 | N-(2,3-dihydro- 1,4-benzodioxin- 6-yl)-4-[4-(2,2,2- trifluoroethoxy)- 6-{[3-(trifluoro- methyl)phenyl]- amino}-1,3,5- triazin-2-yl] piperazine-1- carboxamide | 599.5 | C₂₅H₂₃F₆N₇O₄ |
| 18 | 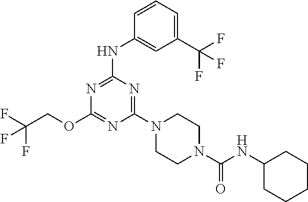 | N-cyclohexyl- 4-[4-(2,2,2- trifluoroethoxy)- 6-{[3-(trifluoro- methyl)phenyl] amino}-1,3,5- triazin-2-yl]- piperazine- 1-carboxamide | 547.5 | C₂₃H₂₇F₆N₇O₂ |
| 19 | 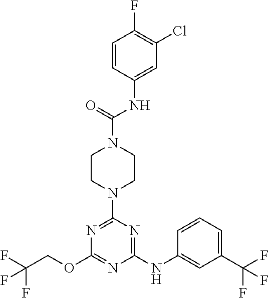 | N-(3-chloro-4- fluorophenyl)- 4-[4-(2,2,2- trifluoroethoxy)- 6-{[3-(trifluoro- methyl)phenyl]- amino}-1,3,5- triazin-2-yl] piperazine- 1-carboxamide | 593.9 | C₂₃H₁₉ClF₇N₇O₂ |
| 20 | 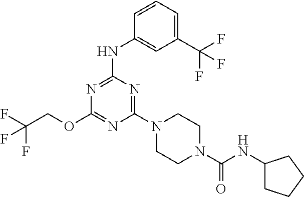 | N-cyclopentyl- 4-[4-(2,2,2- trifluoroethoxy)- 6-{[3-(trifluoro- methyl)phenyl] amino}-1,3,5- triazin-2-yl] piperazine-1- carboxamide | 533.5 | C₂₂H₂₅F₆N₇O₂ |
| 21 | 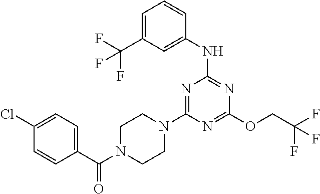 | (4-chlorophenyl) {4-[4-(2,2,2-tri- fluoroethoxy)- 6-{[3-(trifluoro- methyl)phenyl] amino}-1,3,5- triazin-2-yl] piperazin-1- yl}methanone | 560.9 | C₂₃H₁₉ClF₆N₆O₂ |
| 22 | 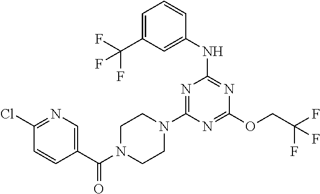 | (6-chloropyridin- 3-yl){4-[4-(2,2,2- trifluoroethoxy)- 6-{[3-(trifluoro- methyl)phenyl] amino}-1,3,5- triazin-2-yl] piperazin-1- yl}methanone | 561.9 | C₂₂H₁₈ClF₆N₇O₂ |
| 23 | 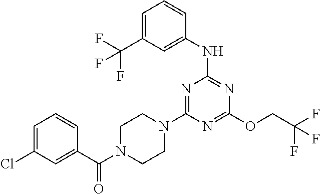 | (3-chlorophenyl) {4-[4-(2,2,2- trifluoroethoxy)- 6-{[3-(trifluoro- methyl)phenyl] amino}-1,3,5- triazin-2-yl] piperazin-1- yl}methanone | 560.9 | C₂₃H₁₉ClF₆N₆O₂ |
| 24 | 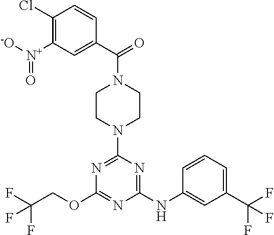 | (4-chloro-3- nitrophenyl){4- [4-(2,2,2-tri- fluoro-ethoxy)- 6-{[3-(trifluoro- methyl)phenyl] amino}-1,3,5- triazin-2-yl] piperazin-1- yl}methanone | 605.9 | C₂₃H₁₈ClF₆N₇O₄ |
| 25 | 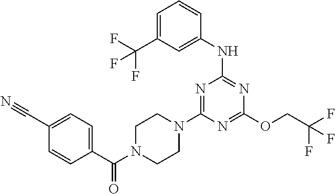 | 4-({4-[4-(2,2,2- trifluoroethoxy)- 6-{[3-(trifluoro- methyl)phenyl] amino}-1,3,5- triazin-2-yl] piperazin-1- yl}carbonyl) benzonitrile | 551.4 | C₂₄H₁₉F₆N₇O₂ |
| 26 | 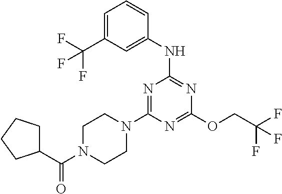 | cyclopentyl{4- [4-(2,2,2- trifluoroethoxy)- 6-{[3-(trifluoro- methyl)phenyl] amino}-1,3,5- triazin-2-yl] piperazin-1-yl} methanone | 518.5 | C₂₂H₂₄F₆N₆O₂ |
| 27 | 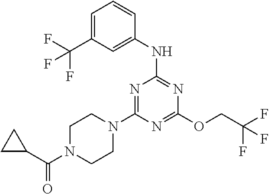 | cyclopropyl{4- [4-(2,2,2- trifluoroethoxy)- 6-{[3-(trifluoro- methyl)phenyl] amino}-1,3,5- triazin-2-yl] piperazin-1- yl}methanone | 490.4 | C₂₀H₂₀F₆N₆O₂ |
| 28 | 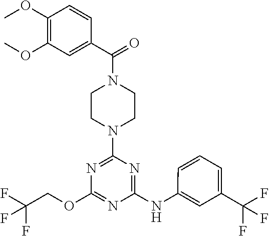 | (3,4-dimethoxy- phenyl){4-[4- (2,2,2-trifluoro- ethoxy)-6-{[3- (trifluoromethyl) phenyl]amino}- 1,3,5-triazin-2- yl]piperazin- 1-yl}methanone | 586.5 | C₂₅H₂₄F₆N₆O₄ |
| 29 | 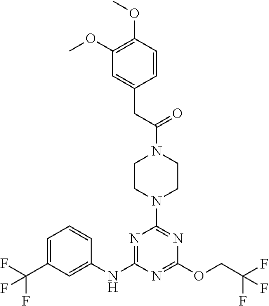 | 2-(3,4-dimethoxy- phenyl)-1-{4-[4- (2,2,2-trifluoro- ethoxy)-6-{[3- (trifluoromethyl) phenyl]amino}- 1,3,5-triazin-2- yl]piperazin-1- yl}ethanone | 600.5 | C₂₆H₂₆F₆N₆O₄ |
| 30 | 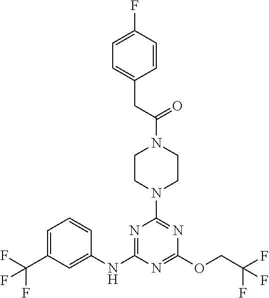 | 2-(4-fluoro- phenyl)-1-{4-[4- (2,2,2-trifluoro- ethoxy)-6-{[3- (trifluoromethyl) phenyl]amino}- 1,3,5-triazin-2- yl]piperazin- 1-yl}ethanone | 558.5 | C₂₄H₂₁F₇N₆O₂ |
| 31 | 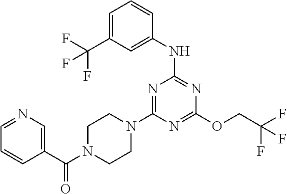 | pyridin-3-yl{4- [4-(2,2,2-tri- fluoroethoxy)-6- {[3-(trifluoro- methyl)phenyl] amino}-1,3,5- triazin-2- yl]piperazin- 1-yl}methanone | 527.4 | C₂₂H₁₉F₆N₇O₂ |
| 32 | 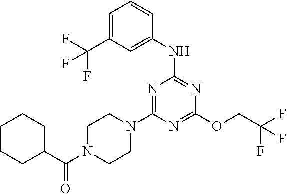 | cyclohexyl{4-[4- (2,2,2-trifluoro- ethoxy)-6-{[3- (trifluoromethyl) phenyl]amino}- 1,3,5-triazin-2- yl]piperazin- 1-yl}methanone | 532.5 | C₂₃H₂₆F₆N₆O₂ |
| 33 | 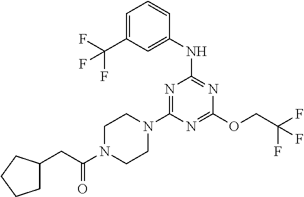 | 2-cyclopentyl- 1-{4-[4-(2,2,2- trifluoroethoxy)- 6-{[3-(trifluoro- methyl)phenyl] amino}-1,3,5- triazin-2-yl] piperazin-1-yl} ethanone | 532.5 | C₂₃H₂₆F₆N₆O₂ |
| 34 | 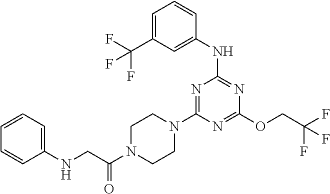 | 2-(phenylamino)- 1-{4-[4-(2,2,2- trifluoroethoxy)- 6-{[3-(trifluoro- methyl)phenyl] amino}-1,3,5- triazin-2-yl] piperazin-1-yl} ethanone | 555.5 | C₂₄H₂₃F₆N₇O₂ |
| 35 | 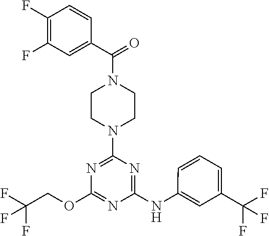 | (3,4-difluoro- phenyl){4-[4- (2,2,2-trifluoro- ethoxy)-6-{[3- (trifluoromethyl) phenyl]amino}- 1,3,5-triazin-2-yl] piperazin- 1-yl}methanone | 562.4 | C₂₃H₁₈F₈N₆O₂ |
| 36 | 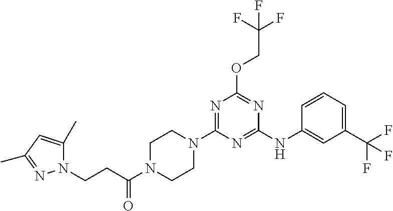 | 3-(3,5-dimethyl- 1H-pyrazol-1-yl)- 1-{4-[4-(2,2,2- tri-fluoroethoxy)- 6-{[3-(trifluoro- methyl)phenyl] amino}-1,3,5- triazin-2-yl] piperazin-1-yl} propan-1-one | 572.5 | C₂₄H₂₆F₆N₈O₂ |
| 37 | 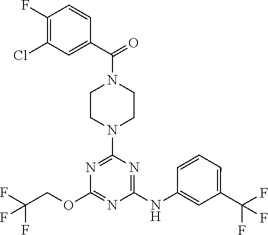 | (3-chloro-4-fluoro- phenyl){4-[4- (2,2,2-trifluoro- ethoxy)-6-{[3- (trifluoroethoxy)- 6-{[3-(trifluoro- methyl)phenyl]- amino}-1,3,5- triazin-2-yl] piperazin-1-yl} methanone | 578.9 | C₂₃H₁₈ClF₇N₆O₂ |
| 38 | 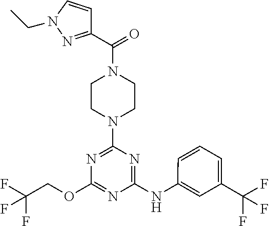 | (1-ethyl-1H- pyrazol-3-yl){4- [4-(2,2,2- trifluoroethoxy)- 6-{[3-(trifluoro- methyl)phenyl]- amino}-1,3,5- triazin-2-yl] piperazin-1- yl}methanone | 544.5 | C₂₂H₂₂F₆N₈O₂ |
| 39 | 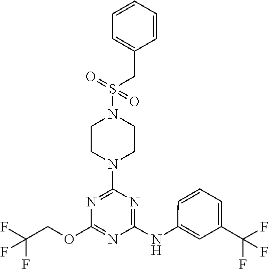 | 4-[4-(benzyl- sulfonyl)piperazin- 1-yl]-6-(2,2,2- trifluoroethoxy)- N-[3-(trifluoro- methyl)phenyl]- 1,3,5-triazin-2- amine | 577.5 | C₂₃H₂₂F₆N₆O₃S |
| 40 | 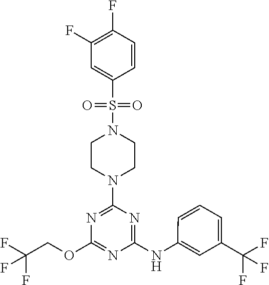 | 4-{4-[(3,4- difluorophenyl) sulfonyl]piperazin- 1-yl}-6- (2,2,2-trifluoro- ethoxy)-N-[3- (trifluoromethyl) phenyl]-1,3,5- triazin-2-amine | 599.5 | C₂₂H₁₈F₈N₆O₃S |
| 41 | 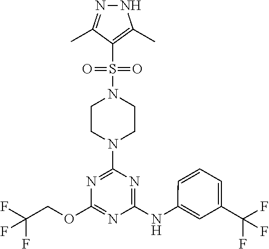 | 4-{4-[(3,5- dimethyl-1H- pyrazol-4-yl) sulfonyl]piperazin- 1-yl}-6-(2,2,2- trifluoroethoxy)- N-[3-(trifluoro- methyl)phenyl]- 1,3,5-triazin- 2-amine | 581.5 | C₂₁H₂₂F₆N₈O₃S |
| 42 | 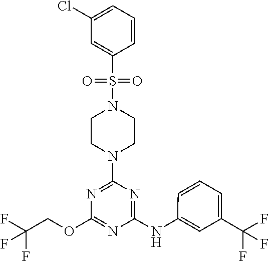 | 4-{4-[(3-chloro- phenyl)sulfonyl] piperazin-1-yl}- 6-(2,2,2-trifluoro- ethoxy)-N-[3- (trifluoromethyl) phenyl]-1,3,5- triazin-2-amine | 598.0 | C₂₂H₁₉ClF₆N₆O₃S |
| 43 | 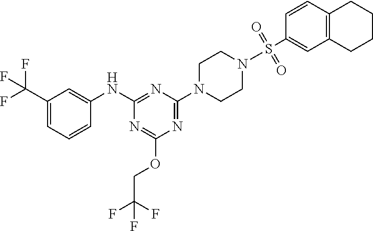 | 4-[4-(5,6,7,8- tetrahydro- naphthalen-2- ylsulfonyl) piperazin-1-yl]- 6-(2,2,2- trifluoroethoxy)- N-[3-(trifluoro- methyl)phenyl]- 1,3,5-triazin-2- amine | 617.6 | C₂₆H₂₆F₆N₆O₃S |
| 44 | 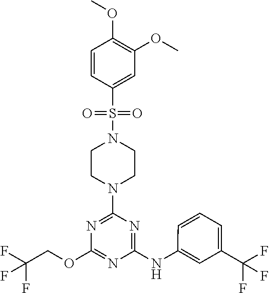 | 4-{4-[(3,4- dimethoxy- phenyl)sulfonyl] piperazin-1-yl}- 6-(2,2,2- trifluoroethoxy)- N-[3-(trifluoro- methyl)phenyl]- 1,3,5-triazin- 2-amine | 623.6 | C₂₄H₂₄F₆N₆O₅S |
| 45 | 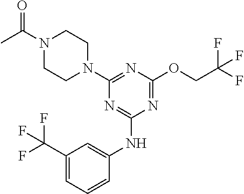 | 1-{4-[4-(2,2,2- trifluoroethoxy)- 6-{[3-(trifluoro- methyl)phenyl] amino}-1,3,5- triazin-2-yl] piperazin-1-yl} ethanone | 464.4 | C₁₈H₁₈F₆N₆O₂ |
| 46 | 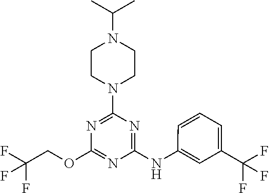 | 4-[4-(propan-2- yl)piperazin- 1-yl]-6-(2,2,2- trifluoroethoxy)- N-[3-(trifluoro- methyl)phenyl]- 1,3,5-triazin- 2-amine | 464.4 | C₁₉H₂₂F₆N₆O |
| 47 | 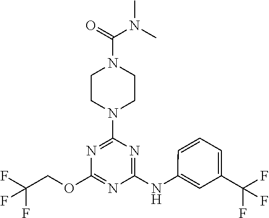 | N,N-dimethyl- 4-[4-(2,2,2- trifluoroethoxy)- 6-{[3-(trifluoro- methyl)phenyl] amino}-1,3,5- triazin-2-yl] piperazine-1- carboxamide | 493.4 | C₁₉H₂₁F₆N₇O₂ |
| 48 | 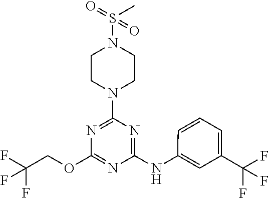 | 4-[4-(methyl- sulfonyl)piperazin- 1-yl]-6-(2,2,2- trifluoroethoxy)- N-[3- (trifluoromethyl) phenyl]-1,3,5- triazin-2-amine | 501.4 | C₁₇H₁₈F₆N₆O₃S |
| 49 | 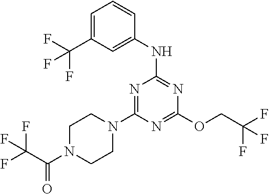 | 2,2,2-trifluoro- 1-{4-[4-(2,2,2- trifluoroethoxy)- 6-{[3- (trifluoromethyl) phenyl]amino}- 1,3,5-triazin- 2-yl]piperazin- 1-yl}ethanone | 518.3 | C₁₈H₁₅F₉N₆O₂ |
| 50 | 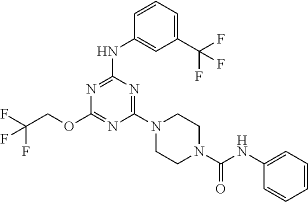 | N-phenyl-4-[4- (2,2,2-trifluoro- ethoxy)-6-{[3- (trifluoromethyl) phenyl]amino}- 1,3,5-triazin-2-yl] piperazine-1- carboxamide | 541.5 | C₂₃H₂₁F₆N₇O₂ |
| 51 | 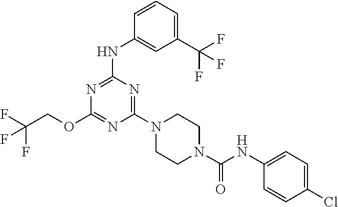 | N-(4-chloro- phenyl)-4-[4- (2,2,2-trifluoro- ethoxy)-6-{[3- (trifluoromethyl) phenyl]amino}- 1,3,5-triazin-2- yl]piperazine- 1-carboxamide | 575.9 | C₂₃H₂₀ClF₆N₇O₂ |
| 52 | 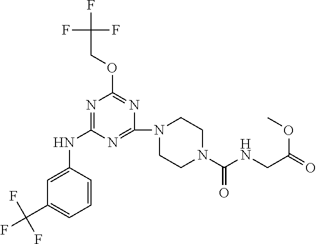 | methyl N-{[4-(4- (2,2,2-trifluoro- ethoxy)-6-{[3- (trifluoromethyl) phenyl]amino}- 1,3,5-triazin-2-yl) piperazin-1-yl] carbonyl} glycinate | 537.4 | C₂₀H₂₁F₆N₇O₄ |
| 53 | 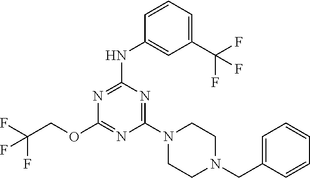 | 4-(4-benzyl- piperazin-1- yl)-6-(2,2,2- trifluoro- ethoxy)-N-[3- (trifluoro- methyl)phenyl]- 1,3,5-triazin- 2-amine | 512.5 | C₂₃H₂₂F₆N₆O |
| 54 | 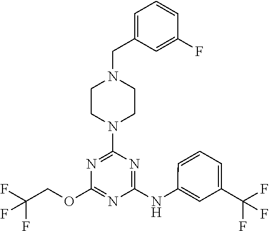 | 4-[4-(3-fluoro- benzyl)piperazin- 1-yl]-6-(2,2,2- trifluoroethoxy)- N-[3-(trifluoro- methyl)phenyl]- 1,3,5-triazin-2- amine | 530.4 | C₂₃H₂₁F₇N₆O |
| 55 | 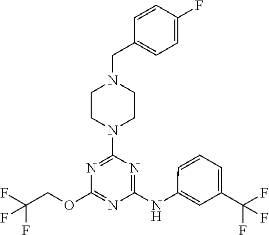 | 4-[4-(4-fluoro- benzyl)piperazin- 1-yl]-6-(2,2,2- trifluoroethoxy)- N-[3-(trifluoro- methyl)phenyl]- 1,3,5-triazin-2- amine | 530.4 | C₂₃H₂₁F₇N₆O |
| 56 | 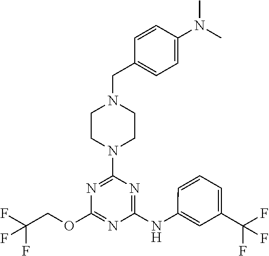 | 4-{4-[4-(dimethyl- amino)benzyl] piperazin-1-yl}- 6-(2,2,2-trifluoro- ethoxy)-N-[3- (trifluoromethyl) phenyl]-1,3,5- triazin-2-amine | 555.5 | C₂₅H₂₇F₆N₇O |
| 57 | 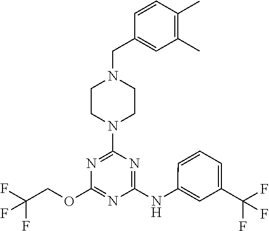 | 4-[4-(3,4-di- methylbenzyl) piperazin- 1-yl]-6-(2,2,2- trifluoroethoxy)- N-[3-(trifluoro- methyl)phenyl]- 1,3,5-triazin-2- amine | 540.5 | C₂₅H₂₇F₆N₇O₃S |
| 58 | 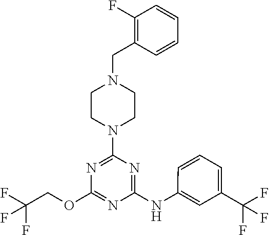 | 4-[4-(2-fluoro- benzyl)piperazin- 1-yl]-6-(2,2,2- trifluoroethoxy)- N-[3-(trifluoro- methyl)phenyl]- 1,3,5-triazin-2- amine | 530.4 | C₂₃H₂₁F₇N₆O |
| 59 | 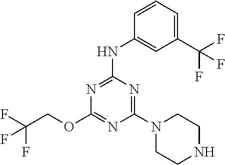 | 4-(piperazin-1- yl)-6-(2,2,2- trifluoroethoxy)- N-[3-(trifluoro- methyl)phenyl]- 1,3,5-triazin- 2-amine | 422.3 | C₁₆H₁₆F₆N₆O |
| 60 | 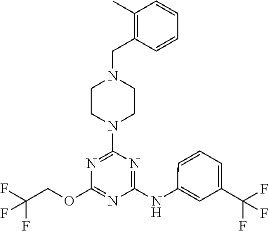 | 4-[4-(2-methyl- benzyl)piperazin- 1-yl]-6-(2,2,2- trifluoroethoxy)- N-[3-(trifluoro- methyl)phenyl]- 1,3,5-triazin- 2-amine | 526.5 | C₂₄H₂₄F₆N₆O |
| 61 | 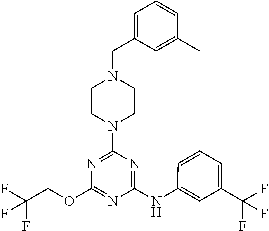 | 4-[4-(3-methyl- benzyl)piperazin- 1-yl]-6-(2,2,2- trifluoroethoxy)- N-[3-(trifluoro- methyl)phenyl]- 1,3,5-triazin-2- amine | 526.5 | C₂₄H₂₄F₆N₆O |
| 62 | 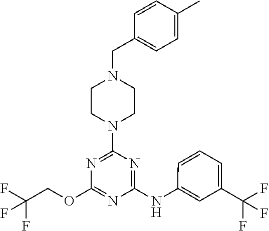 | 4-[4-(4-methyl- benzyl)piperazin- 1-yl]-6-(2,2,2- trifluoroethoxy)- N-[3-(trifluoro- methyl)phenyl]- 1,3,5-triazin-2- amine | 526.5 | C₂₄H₂₄F₆N₆O |
| 63 | 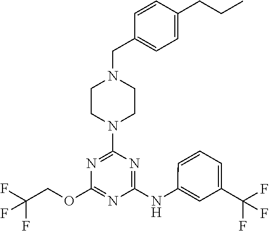 | 4-[4-(4-propyl- benzyl)piperazin- 1-yl]-6-(2,2,2- trifluoroethoxy)- N-[3-(trifluoro- methyl)phenyl]- 1,3,5-triazin-2- amine | 554.5 | C₂₆H₂₈F₆N₆O |
| 64 | 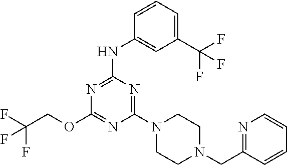 | 4-[4-(pyridin- 2-ylmethyl) piperazin-1-yl]- 6-(2,2,2-tri- fluoroethoxy)- N-[3-(trifluoro- methyl)phenyl]- 1,3,5-triazin-2- amine | 513.4 | C₂₂H₂₁F₆N₇O |
| 65 | 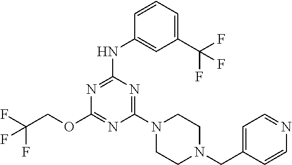 | 4-[4-(pyridin- 4-ylmethyl) piperazin-1-yl]- 6-(2,2,2- trifluoroethoxy)- N-[3-(trifluoro- methyl)phenyl]- 1,3,5-triazin-2- amine | 513.4 | C₂₂H₂₁F₆N₇O |
| 66 | 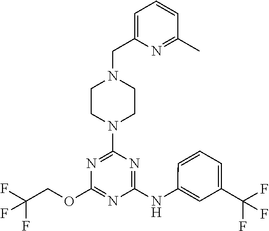 | 4-{4-[(6-methyl- pyridin-2-yl) methyl] piperazin-1- yl}-6-(2,2,2- trifluoro- ethoxy)-N- [3-(trifluoro- methyl)phenyl]- 1,3,5-triazin- 2-amine | 527.5 | C₂₃H₂₃F₆N₇O |
| 67 | 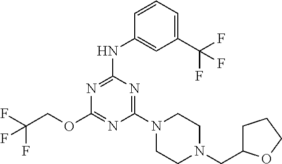 | 4-(4-((tetra- hydrofuran- 2-yl)methyl) piperazin-1- yl)-6-(2,2,2-tri- fluoroethoxy)- N-(3-(tri- fluoromethyl) phenyl)-1,3,5- triazin-2-amine | 506.4 | C₂₁H₂₄F₆N₆O₂ |
| 68 | 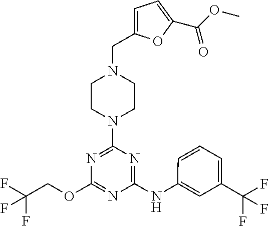 | methyl 5-({4- [4-(2,2,2-tri- fluoroethoxy)- 6-{[3-(trifluoro- methyl)phenyl] amino}-1,3,5- triazin-2-yl] piperazin-1-yl} methyl)furan- 2-carboxylate | 560.5 | C₂₃H₂₂F₆N₆O₄ |
| 69 | 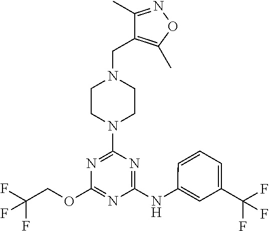 | 4-{4-[(3,5-di- methylisoxazol- 4-yl)methyl] piperazin-1-yl}- 6-(2,2,2- trifluoroethoxy)- N-[3- (trifluoro- methyl)phenyl]- 1,3,5-triazin-2- amine | 531.5 | C₂₂H₂₃F₆N₇O₂ |
| 70 | 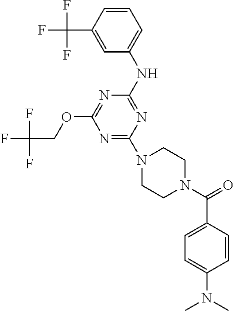 | 4-{4-[4-(di- methylamino) benzoyl] piperazin-1-yl}- 6-(2,2,2- trifluoroethoxy)- N-[3-(trifluoro- methyl)phenyl]- 1,3,5-triazin- 2-amine | 569.5 | C₂₅H₂₅F₆N₇O₂ |

### Procedures and Analytical Data for Table 34.

Entries 5 to 70 were prepared by the procedures described above.

### [4-Piperazin-1-yl-6-(2,2,2-trifluoro-ethoxy)-[1,3,5]triazin-2-yl]-(3-trifluoro-methyl-phenyl)-amine hydrochloride

¹H-NMR (400 MHz, DMSO-D₆) δ_{H}: 3.15 (4H, broad peak, Z/E forms), 4.11 (4H, broad peak, Z/E forms), 5.05 (1H, q, J=7.5 Hz), 7.35 (1H, d, J=8.5 Hz), 7.59 (1H, t, J=8.5 Hz), 7.85 (1H, d, J=8.5 Hz), 8.15 (1H, s), 9.51 (1H, broad peak, Z/E forms), 10.11 (1H, s). MW 422.33. LCMS t_{R} (min): 1.60. MS (APCI+), m/z 423.05 [M+H]⁺ HPLC t_{R} (min): 11.47. Mₚ 248-250° C.

### 1. 1-Methyl-4-[4-(2,2,2-trifluoro-ethoxy)-6-(3-trifluoromethyl-phenylamino)-[1,3,5]triazin-2-yl]-piperazin-2-one

Yield 117 mg, 46%. ¹H-NMR (400 MHz, DMSO-D₆) δ_{H}: 2.90 (3H, s), 3.43 (2H, broad), 4.00 (2H, broad t, J=7.5 Hz), 4.30 (2H, s), 5.01 (2H, q, J=7.5 Hz), 7.35 (1H, d, J=8.5 Hz), 7.53 (1H, t, J=8.5 Hz), 7.84 (1H, d, J=8.5 Hz), 8.23 (1H, s), 10.07 (1H, broad). MW 450.35. LCMS t_{R} (min): 1.95. MS (APCI), m/z 451.07 [M+H]⁺. HPLC t_{R} (min): 14.70. M_{P} 147-149° C.

### 2. (4-Dimethylamino-phenyl)-{4-[4-(2,2,2-trifluoro-ethoxy)-6-(3-trifluoromethyl-phenylamino)-[1,3,5]triazin-2-yl]-piperazin-1-yl}-methanone

A mixture of compound 31 as hydrochloride (300 mg, 0.65 mmol), 4-dimethylamino-benzoic acid (119 mg, 0.72 mmol), TBTU (241 mg, 0.75 mmol), Et₃N (133 mg, 1.30 mmol) in acetonitrile (10 mL) was stirred at 50° C. for 3 hours, diluted with 40% aqueous solution of K₂CO₃, extracted with ethyl acetate. The combined organic phases were washed with water, dried over sodium sulfate and concentrated. The residue was purified by column chromatography on silica gel (5→15% ethyl acetate/dichloromethane) and triturated with hexane giving an acid as white crystals.

A mixture of the acid (260 mg, 0.47 mmol), dimethylamine hydrochloride (76 mg, 0.93 mmol), TBTU (225 mg, 0.70 mmol) and Et₃N (142 mg, 1.40 mmol) in acetonitrile (10 ml) was stirred at 50° C. for 5 hours, diluted with 40% aqueous solution of K₂CO₃, extracted with ethyl acetate. The combined organic phases were washed with water, dried over sodium sulfate and concentrated. The residue was purified by column chromatography on silica gel (ethyl acetate/dichloromethane) and triturated with hexane giving the target compound as cream crystals. Yield 87 mg, 18% (for two steps). ¹H-NMR (400 MHz, DMSO-D₆) δ_{H}: 2.49 (4H, broad peak, Z/E forms), 2.91 (6H, s), 3.61 (2H, s), 3.81 (4H, broad peak, Z/E forms), 5.01 (2H, q, J=7.5 Hz), 7.31 (1H, d, J=8.5 Hz), 7.38 (4H, superposition of two d), 7.51 (1H, t, J=8.5 Hz), 7.81 (1H, d, J=8.5 Hz), 8.21 (1H, s), 9.95 (1H, broad peak, Z/E forms).

MW 583.54. LCMS t_{R} (min): 1.62. MS (APCI+), m/z 584.58, 585.90 [M+H]⁺. HPLC t_{R} (min): 12.24. Mₚ 210-212° C.

### 3. 4-{4-[3-(2,2,2-Trifluoro-ethoxy)-5-(3-trifluoromethyl-phenylamino)-phenyl]-piperazin-1-ylmethyl}-benzoic acid

To a suspension of compound 31 (400 mg, 0.87 mmol) in dichloroethane (20 mL), a 4-formyl-benzoic acid (137 mg, 0.91 mmol), acetic acid (0.02 mL), NaHB(OAc)₃ (739 mg, 3.49 mmol) and Et₃N (88 mg, 0.87 mmol) were added. The reaction mixture was stirred at room temperature for 16 hours. The resulting mixture was poured into aqueous solution NaHCO₃, stirred for 3 hours and extracted with dichloromethane (2×35 mL). The combined organic phases were washed with water, dried over sodium sulfate and concentrated. The residue was purified by column chromatography on silica gel (10-35%, methanol/ethyl acetate) and triturated with hexane giving yellowish crystals (269 mg). According to LCMS of the product the material consisted of a mixture of the target compound (50%). LCMS t_{R} (min): 1.65, MS (APCI+), m/z 557.02 [M+H]⁺. The material was used on the next stage without additional purification

### 4. [4-[4-(4-Dimethylamino-benzenesulfonyl)-piperazin-1-yl]-6-(2,2,2-trifluoro-ethoxy)-[1,3,5]-triazin-2-yl]-(3-trifluoromethyl-phenyl)-amine

¹H-NMR (400 MHz, DMSO-D₆) δ_{H}: 2.98 (4H, broad peak, Z/E forms), 2.99 (6H, s), 3.82 (4H, broad peak, Z/E forms), 4.95 (1H, q, J=7.5 Hz), 6.78 (1H, d, J=8.5 Hz), 7.51 (3H, superposition of d (2H) and t (1H)), 7.81 (1H, d, J=8.5 Hz), 8.15 (1H, s), 9.99 (1H, broad peak, Z/E forms). MW 605.57. LCMS t_{R} (min): 2.17. MS (APCI+), m/z 606.05 [M+H]⁺. HPLC t_{R} (min): 18.47. Mp 136-137° C.

### 5. 4-(4-methylpiperazin-1-yl)-6-(2,2,2-trifluoroethoxy)-N-[3-(trifluoromethyl)phenyl]-1,3,5-triazin-2-amine

LCMS: M+1=436.3; 1H NMR (DMSO-d6, 90° C., ppm): d=2.30 s (3H), 2.98 t (4H), 3.82 t (4H), 4.92 q (2H), 7.34 d (1H), 7.48 t (1H), 7.86 d (1H), 8.28 s (1H), 9.64 s (1H).

### 6. 4-[4-(2-chlorobenzyl)piperazin-1-yl]-6-(2,2,2-trifluoroethoxy)-N-[3-(trifluoromethyl)phenyl]-1,3,5-triazin-2-amine

LCMS: M+1=546.9; 1H NMR (DMSO-d6, 90° C., ppm): d=2.58 t (4H), 3.68 s (2H), 3.84 t (4H), 4.92 q (2H), 7.42 m (5H), 7.82 d (1H), 8.22 s (1H), 9.70 s (1H).

### 7. 4-[4-(2,3-dihydro-1,4-benzodioxin-6-ylmethyl)piperazin-1-yl]-6-(2,2,2-trifluoroethoxy)-N-[3-(trifluoromethyl)phenyl]-1,3,5-triazin-2-amine

LCMS: M+1=570.4; 1H NMR (DMSO-d6, 90° C., ppm): d=3.02 t (2H), 3.42 t (2H), 3.76 t (4H), 4.22 t (4H), 4.92 q (2H), 6.82 d (3H), 7.28 d (1H), 7.46 t (1H), 7.78 d (1H), 8.18 (1H), 9.52 s (1H).

### 8. 4-[4-(2,4-difluorobenzyl)piperazin-1-yl]-6-(2,2,2-trifluoroethoxy)-N-[3-(trifluoromethyl)phenyl]-1,3,5-triazin-2-amine

LCMS: M+1=548.4; 1H NMR (DMSO-d6, 90° C., ppm): d=3.02 t (4H), 3.62 s (2H), 4.78 t (4H), 4.92 q (2H), 7.16 m (2H), 7.32 d (1H), 7.48 m (2H), 7.84 d (1H), 8.22 s (1H), 9.74 s (1H).

### 9. 4-(2,2,2-trifluoroethoxy)-N-[3-(trifluoromethyl)phenyl]-6-[4-(3,4,5-trimethoxybenzyl)piperazin-1-yl]-1,3,5-triazin-2-amine

LCMS: M+1=602.5; 1H NMR (DMSO-d6, 90° C., ppm): d=3.58 t (4H), 3.84 t (4H), 4.92 q (2H), 6.72 m (1H), 7.36 m (5H), 7.88 d (1H), 8.22 s (1H), 8.54 s (1H), 9.76 s (1H).

### 10. 4-{4-[3-(benzyloxy)benzyl]piperazin-1-yl}-6-(2,2,2-trifluoroethoxy)-N-[3-(trifluoromethyl)phenyl]-1,3,5-triazin-2-amine

LCMS: M+1=618.5; 1H NMR (DMSO-d6, 90° C., ppm): d=2.46 t (4H), 3.52 s (2H), 3.80 t (4H), 5.04 m (4H), 6.96 m (3H), 7.36 m (8H), 8.82 s (1H), 8.28 s (1H), 10.08 s (1H).

### 11. 4-(2,2,2-trifluoroethoxy)-6-{4-[2-(trifluoromethyl)benzyl]piperazin-1-yl}-N-[3-(trifluoromethyl)phenyl]-1,3,5-triazin-2-amine

LCMS: M+1=580.4; 1H NMR (DMSO-d6, 90° C., ppm): d=3.02 t (4H), 3.72 s (2H), 3.82 t (4H), 4.92 q (2H), 7.30 d (1H), 7.48 t (2H), 7.66 m (2H), 7.84 t (2H), 8.22 s (1H), 9.72 s (1H).

### 12. 4-(2,2,2-trifluoroethoxy)-6-{4-[3-(trifluoromethyl)benzyl]piperazin-1-yl}-N-[3-(trifluoromethyl)phenyl]-1,3,5-triazin-2-amine

LCMS: M+1=580.4; 1H NMR (DMSO-d6, 90° C., ppm): d=2.46 t (4H), 3.68 s (2H), 3.82 t (4H), 4.92 q (2H), 7.34 d (1H), 7.72 m (6H), 8.22 t (1H), 9.72 s (1H).

### 13. N-(3-fluorophenyl)-4-[4-(2,2,2-trifluoroethoxy)-6-{[3-(trifluoromethyl)phenyl]amino}-1,3,5-triazin-2-yl]piperazine-1-carboxamide

LCMS: M+1=559.4; NMR 1H, DMSO-d6 δ, ppm: 1.10 bm (1H); 1.50 m (2H); 1.85 d (2H); 2.85 m (2H); 4.00 m (3H); 4.30 d (2H); 4.95 m (2H); 6.75 t (1H); 7.15-7.45 m (6H); 7.52 t (1H); 7.92 bm (1H); 8.20 bm (1H); 9.50 bs (1H).

### 14. 4-[4-(2,2,2-trifluoroethoxy)-6-{[3-(trifluoromethyl)phenyl]amino}-1,3,5-triazin-2-yl]-N-[3-(trifluoromethyl)phenyl]piperazine-1-carboxamide

LCMS: M+1=609.4; NMR 1H, DMSO-d6 δ, ppm: 1.10 bm (1H); 1.50 m (2H); 1.85 d (2H); 2.85 m (4H); 3.30 t (2H); 3.95 m (3H); 4.95 m (2H); 6.15 t (1H); 7.15-7.45 m (6H); 7.52 t (1H); 7.92 bm (1H); 8.15 bm (1H); 9.50 bs (1H).

### 15. N-benzyl-4-[4-(2,2,2-trifluoroethoxy)-6-{[3-(trifluoromethyl)phenyl]amino}-1,3,5-triazin-2-yl]piperazine-1-carboxamide

LCMS: M+1=555.4; NMR 1H, DMSO-d6 δ, ppm: 1.50 m (6H); 1.65 m (2H); 1.83 m (4H); 2.85 m (2H); 3.97 m (4H); 4.95 m (2H); 5.85 d (1H); 7.35 m (2H); 7.52 t (1H); 7.92 bm (1H); 8.15 bm (1H); 9.50 bs (1H).

### 16. N-(2-phenylethyl)-4-[4-(2,2,2-trifluoroethoxy)-6-{[3-(trifluoromethyl)phenyl]amino}-1,3,5-triazin-2-yl]piperazine-1-carboxamide

LCMS: M+1=569.5; NMR 1H, DMSO-d6 δ, ppm: 3.60 t (4H); 3.80 t (4H); 4.95 m (2H); 6.75 m (1H); 7.20-7.50 m (5H); 7.85 d (1H); 8.15 s (1H); 8.30 s (1H); 9.80 bs (1H).

### 17. N-(2,3-dihydro-1,4-benzodioxin-6-yl)-4-[4-(2,2,2-trifluoroethoxy)-6-{[3-(trifluoromethyl)phenyl]amino}-1,3,5-triazin-2-yl]piperazine-1-carboxamide

LCMS: M+1=599.4; NMR 1H, DMSO-d6 δ, ppm: 3.50 t (4H); 3.80 t (4H); 4.30 d (2H); 4.95 m (2H); 6.75 m (1H); 7.10-7.30 m (5H); 7.50 t (1H); 7.85 d (1H); 8.20 s (1H); 9.80 bs (1H).

### 18. N-cyclohexyl-4-[4-(2,2,2-trifluoroethoxy)-6-{[3-(trifluoromethyl)phenyl]amino}-1,3,5-triazin-2-yl]piperazine-1-carboxamide

LCMS: M+1=547.5; NMR 1H, DMSO-d6 δ, ppm: 2.75 t (2H); 3.40 t (2H); 3.50 t (4H); 3.80 t (4H); 4.95 m (2H); 6.40 m (1H); 7.10-7.30 m (6H); 7.50 t (1H); 7.85 d (1H); 8.20 s (1H); 9.80 bs (1H).

### 19. N-(3-chloro-4-fluorophenyl)-4-[4-(2,2,2-trifluoroethoxy)-6-{[3-(trifluoromethyl)phenyl]amino}-1,3,5-triazin-2-yl]piperazine-1-carboxamide

LCMS: M+1=593.8; NMR 1H, DMSO-d6 δ, ppm: 1.10-1.40 m (5H); 1.60-1.80 m (5H); 3.50 m (5H); 3.80 t (4H); 4.95 m (2H); 5.80 d (1H); 7.30 d (1H); 7.50 t (1H); 7.85 d (1H); 8.20 s (1H); 9.80 bs (1H).

### 20. N-cyclopentyl-4-[4-(2,2,2-trifluoroethoxy)-6-{[3-(trifluoromethyl)phenyl]amino}-1,3,5-triazin-2-yl]piperazine-1-carboxamide

LCMS: M+1=533.4; NMR 1H, DMSO-d6 δ, ppm: 1.40-1.80 m (8H); 3.40 t (4H); 3.80 t (4H); 4.00 m (1H); 4.95 m (2H); 6.00 d (1H); 7.30 d (1H); 7.50 t (1H); 7.85 d (1H); 8.20 s (1H); 9.80 bs (1H).

### 21. (4-chlorophenyl){4-[4-(2,2,2-trifluoroethoxy)-6-{[3-(trifluoromethyl)phenyl]amino}-1,3,5-triazin-2-yl]piperazin-1-yl}methanone

LCMS: M+1=560.8; NMR 1H, DMSO-d6 δ, ppm: 3.60 t (4H); 3.90 t (4H); 4.95 m (2H); 7.30 d (1H); 7.50 m (4H); 7.85 d (1H); 8.20 s (1H); 9.80 bs (1H).

### 22. (6-chloropyridin-3-yl){4-[4-(2,2,2-trifluoroethoxy)-6-{[3-(trifluoromethyl)phenyl]amino}-1,3,5-triazin-2-yl]piperazin-1-yl}methanone

LCMS: M+1=561.8; NMR 1H, DMSO-d6 δ, ppm: 3.60 t (4H); 3.90 t (4H); 4.95 m (2H); 7.30 d (1H); 7.50 d (1H); 7.55 m (4H); 7.85 d (1H); 8.20 s (1H); 9.80 bs (1H).

### 23. (3-chlorophenyl){4-[4-(2,2,2-trifluoroethoxy)-6-{[3-(trifluoromethyl)phenyl]amino}-1,3,5-triazin-2-yl]piperazin-1-yl}methanone

LCMS: M+1=560.8; NMR 1H, DMSO-d6 δ, ppm: 3.60 t (4H); 3.90 t (4H); 4.95 m (2H); 7.30 d (1H); 7.50 t (1H); 7.60 d (2H); 7.90 m (3H); 8.20 s (1H); 9.80 bs (1H).

### 24. (4-chloro-3-nitrophenyl){4-[4-(2,2,2-trifluoroethoxy)-6-{[3-(trifluoromethyl)phenyl]amino}-1,3,5-triazin-2-yl]piperazin-1-yl}methanone

LCMS: M+1=584.6; NMR 1H, DMSO-d6 δ, ppm: 0.92 t (3H); 1.40 m (4H); 2.34 t (2H); 3.12 s (4H); 4.51 d (2H); 4.92 q (2H); 6.74 t (2H); 6.90 s (1H); 7.13 t (1H); 7.23 d (1H); 7.49 t (1H); 7.90 m (2H); 8.16 s (1H); 9.58 s (1H).

### 25. 4-({4-[4-(2,2,2-trifluoroethoxy)-6-{[3-(trifluoromethyl)phenyl]amino}-1,3,5-triazin-2-yl]piperazin-1-yl}carbonyl)benzonitrile

LCMS: M+1=551.4; NMR 1H, DMSO-d6 δ, ppm: 1.70 m (8H); 3.10 m (1H); 3.60 t (4H); 3.90 t (4H); 4.95 m (2H); 7.35 d (1H); 7.55 t (1H); 7.85 d (1H); 8.20 s (1H); 9.80 bs (1H).

### 26. cyclopentyl{4-[4-(2,2,2-trifluoroethoxy)-6-{[3-(trifluoromethyl)phenyl]amino}-1,3,5-triazin-2-yl]piperazin-1-yl}methanone

LCMS: M+1=518.4; NMR 1H, DMSO-d6 δ, ppm: 0.80 m (4H); 1.97 m (1H); 3.70 t (4H); 3.90 t (4H); 4.95 m (2H); 7.30 d (1H); 7.50 t (1H); 7.80 d (1H); 8.20 s (1H); 9.80 bs (1H).

### 27. cyclopropyl{4-[4-(2,2,2-trifluoroethoxy)-6-{[3-(trifluoromethyl)phenyl]amino}-1,3,5-triazin-2-yl]piperazin-1-yl}methanone

LCMS: M+1=490.4; NMR 1H, DMSO-d6 δ, ppm: 3.50-3.80 m (16H); 4.95 m (2H); 6.85 m (3H); 7.30 d (1H); 7.50 t (1H); 7.80 d (1H); 8.20 s (1H); 9.80 bs (1H).

### 28. (3,4-dimethoxyphenyl){4-[4-(2,2,2-trifluoroethoxy)-6-{[3-(trifluoromethyl)phenyl]amino}-1,3,5-triazin-2-yl]piperazin-1-yl}methanone

LCMS: M+1=586.4; NMR 1H, DMSO-d6 δ, ppm: 3.60 t (4H); 3.90 m (6H); 4.95 m (2H); 7.05 m (2H); 7.30 m (3H); 7.50 t (1H); 7.80 d (1H); 8.20 s (1H); 9.80 bs (1H).

### 29. 2-(3,4-dimethoxyphenyl)-1-{4-[4-(2,2,2-trifluoroethoxy)-6-{[3-(trifluoromethyl)phenyl]amino}-1,3,5-triazin-2-yl]piperazin-1-yl}ethanone

LCMS: M+1=600.5; NMR 1H, DMSO-d6 δ, ppm: 3.60 t (4H); 3.90 t (4H); 4.05 s (2H); 4.95 m (2H); 5.35 bs (1H); 6.65 m (3H); 7.10 t (2H); 7.30 d (1H); 7.50 t (1H); 7.80 d (1H); 8.20 s (1H); 9.80 bs (1H).

### 30. 2-(4-fluorophenyl)-1-{4-[4-(2,2,2-trifluoroethoxy)-6-{[3-(trifluoromethyl)phenyl]amino}-1,3,5-triazin-2-yl]piperazin-1-yl}ethanone

LCMS: M+1=558.4; NMR 1H, DMSO-d6 δ, ppm: 3.60 t (4H); 3.90 t (4H); 4.95 m (2H); 7.30 d (2H); 7.50 m (3H); 7.80 d (1H); 8.20 s (1H); 9.80 bs (1H).

### 31. pyridin-3-yl{4-[4-(2,2,2-trifluoroethoxy)-6-{[3-(trifluoromethyl)phenyl]amino}-1,3,5-triazin-2-yl]piperazin-1-yl}methanone

LCMS: M+1=527.4; NMR 1H, DMSO-d6 δ, ppm: 2.00 s (3H); 2.25 s (3H); 2.85 t (2H); 3.50 t (4H); 3.75 t (4H); 4.95 m (2H); 5.75 s (1H); 7.30 d (2H); 7.50 m (3H); 7.80 d (1H); 8.20 s (1H); 9.80 bs (1H).

### 32. cyclohexyl{4-[4-(2,2,2-trifluoroethoxy)-6-{[3-(trifluoromethyl)phenyl]amino}-1,3,5-triazin-2-yl]piperazin-1-yl}methanone

LCMS: M+1=532.4; NMR 1H, DMSO-d6 δ, ppm: 3.55 t (4H); 3.85 t (4H); 4.95 m (2H); 7.30 d (1H); 7.50 m (3H); 7.80 d (1H); 8.20 s (1H); 9.80 bs (1H).

### 33. 2-cyclopentyl-1-{4-[4-(2,2,2-trifluoroethoxy)-6-{[3-(trifluoromethyl)phenyl]amino}-1,3,5-triazin-2-yl]piperazin-1-yl}ethanone

LCMS: M+1=532.4; NMR 1H, DMSO-d6 δ, ppm: 1.50 t (3H); 3.90 t (8H); 4.20 m (2H); 4.95 m (2H); 6.60 s (1H); 7.30 d (1H); 7.50 m (3H); 7.80 d (1H); 8.20 s (1H); 9.80 bs 1H).

### 34. 2-(phenylamino)-1-{4-[4-(2,2,2-trifluoroethoxy)-6-{[3-(trifluoromethyl)phenyl]amino}-1,3,5-triazin-2-yl]piperazin-1-yl}ethanone

LCMS: M+1=555.4; NMR 1H, DMSO-d6 δ, ppm: 3.40 t (4H); 3.90 t (8H); 4.95 m (2H); 7.30 d (1H); 7.50 m (3H); 7.80 m (5H); 8.20 s (1H); 9.80 bs (1H).

### 35. (3,4-difluorophenyl){4-[4-(2,2,2-trifluoroethoxy)-6-{[3-(trifluoromethyl)phenyl]amino}-1,3,5-triazin-2-yl]piperazin-1-yl}methanone

LCMS: M+1=562.4; NMR 1H, DMSO-d6 δ, ppm: 1.60 m (2H); 1.85 d (2H); 2.20 t (2H); 2.85 d (2H); 3.85 m (1H); 4.95 m (2H); 7.20-7.50 m (6H); 7.80-8.20 m (2H); 9.60 bs (1H).

### 36. 3-(3,5-dimethyl-1H-pyrazol-1-yl)-1-{4-[4-(2,2,2-trifluoroethoxy)-6-{[3-(trifluoromethyl)phenyl]amino}-1,3,5-triazin-2-yl]piperazin-1-yl}propan-1-one

LCMS: M+1=572.5; NMR 1H, DMSO-d6 δ, ppm: 1.60 m (2H); 1.85 d (2H); 2.20 t (2H); 2.85 d (2H); 3.50 s (1H); 3.85 m (1H); 4.95 m (2H); 7.00 m (2H); 7.30-7.50 m (4H); 7.80-8.20 m (2H); 9.60 bs (1H).

### 37. (3-chloro-4-fluorophenyl){4-[4-(2,2,2-trifluoroethoxy)-6-{[3-(trifluoromethyl)phenyl]amino}-1,3,5-triazin-2-yl]piperazin-1-yl}methanone

LCMS: M+1=578.8; NMR 1H, DMSO-d6 δ, ppm: 1.60 m (2H); 1.85 d (2H); 2.20 t (2H); 2.85 d (2H); 3.65 s (2H); 3.85 m (1H); 4.95 m (2H); 7.30-7.50 m (4H); 7.60-8.00 m (4H); 8.20 bs (1H); 9.60 bs (1H).

### 38. (1-ethyl-1H-pyrazol-3-yl){4-[4-(2,2,2-trifluoroethoxy)-6-{[3-(trifluoromethyl)phenyl]amino}-1,3,5-triazin-2-yl]piperazin-1-yl}methanone

LCMS: M+1=544.4; NMR 1H, DMSO-d6 δ, ppm: 1.60 m (2H); 1.85 d (2H); 2.20 t (2H); 2.85 d (2H); 3.55 s (1H); 3.85 m (1H); 4.95 m (2H); 7.30 m (2H); 7.50-7.70 m (5H); 7.80-8.20 m (2H); 9.60 bs (1H).

### 39. 4-[4-(benzylsulfonyl)piperazin-1-yl]-6-(2,2,2-trifluoroethoxy)-N-[3-(trifluoromethyl)phenyl]-1,3,5-triazin-2-amine

LCMS: M+1=576.5; NMR 1H, DMSO-d6 δ, ppm: 1.60 m (2H); 1.85 d (2H); 2.90 d (2H); 4.10 d (3H); 4.95 m (2H); 6.75 m (1H); 7.20-7.60 m (6H); 7.80-8.20 m (2H); 8.50 (1H); 9.60 bs (1H).

### 40. 4-{4-[(3,4-difluorophenyl)sulfonyl]piperazin-1-yl}-6-(2,2,2-trifluoroethoxy)-N-[3-(trifluoromethyl)phenyl]-1,3,5-triazin-2-amine

LCMS: M+1=598.4; NMR 1H, DMSO-d6 δ, ppm: 1.60 m (2H); 1.85 d (2H); 2.90 d (2H); 4.10 d (3H); 4.95 m (2H); 7.20 t (1H); 7.30 d (1H); 7.40-7.60 m (3H); 7.75 d (1H); 7.80-8.20 m (2H); 8.50 s (1H); 9.60 bs (1H).

### 41. 4-{4-[(3,5-dimethyl-1H-pyrazol-4-yl)sulfonyl]piperazin-1-yl}-6-(2,2,2-trifluoroethoxy)-N-[3-(trifluoromethyl)phenyl]-1,3,5-triazin-2-amine

LCMS: M+1=580.5; NMR 1H, DMSO-d6 δ, ppm: 1.60 m (2H); 1.85 d (2H); 3.10 t (2H); 4.10 d (3H); 4.95 m (2H); 7.30-7.50 m (7H); 7.80-8.20 m (2H); 9.60 bs (1H).

### 42. 4-{4-[(3-chlorophenyl)sulfonyl]piperazin-1-yl}-6-(2,2,2-trifluoroethoxy)-N-[3-(trifluoromethyl)phenyl]-1,3,5-triazin-2-amine

LCMS: M+1=596.9; NMR 1H, DMSO-d6 δ, ppm: 1.60 m (2H); 1.85 d (2H); 3.10 t (2H); 4.10 d (3H); 4.95 m (2H); 7.25-7.50 m (4H); 7.60-7.90 m (2H); 8.10 bs (1H); 8.40 bs (1H); 9.50 bs (1H).

### 43. 4-[4-(5,6,7,8-tetrahydronaphthalen-2-ylsulfonyl)piperazin-1-yl]-6-(2,2,2-trifluoroethoxy)-N-[3-(trifluoromethyl)phenyl]-1,3,5-triazin-2-amine

LCMS: M+1=617 NMR 1H, DMSO-d6 δ, ppm: 1/9 bs (4H); 2.48 bs (4H); 2.83 bs (4H); 3.15 bs (4H); 3.87 bs (4H); 4.95 m (2H); 7.28 m (2H); 7.48 m (3H); 7.88 d (1H); 8.12 bs (1H); 9.68 bs (1H).

### 44. 4-{4-[(3,4-dimethoxyphenyl)sulfonyl]piperazin-1-yl}-6-(2,2,2-trifluoroethoxy)-N-[3-(trifluoromethyl)phenyl]-1,3,5-triazin-2-amine

1H NMR (DMSO-d6, 90° C., ppm): δ=2.88 s (4H, 2CH2), 3.10 t (4H. 2CH2), 3.84 (3H, CH3), 3.86 s (3H, CH3), 4.92 q (2H, CH2), 7.15 d (1H, Ar), 7.23 d (1H, Ar), 7.29-7.41 m (2H, Ar), 7.51 t (1H, Ar), 7.83 d (1H, Ar), 8.12 s (1H, Ar), 9.63 s (1H, NH); LC-MS [M+1]: calc'd: 622.6; obs'd: 623.5.

### 45. 1-{4-[4-(2,2,2-trifluoroethoxy)-6-{[3-(trifluoromethyl)phenyl]amino}-1,3,5-triazin-2-yl]piperazin-1-yl}ethanone

LCMS: M+1=464.3; 1H NMR (DMSO-d6, 90° C., ppm): δ=2.0 (3H, s); 3.6 (4H, t); 3.8 (4H, t); 4.9 (2H, q); 7.35 (1H, d); 7.5 (1 Kt); 7.85 (1H, d); 8.2 (1H, s); 9.7 (1H, s).

### 46. 4-[4-(propan-2-yl)piperazin-1-yl]-6-(2,2,2-trifluoroethoxy)-N-[3-(trifluoromethyl)phenyl]-1,3,5-triazin-2-amine

LCMS: M+1=464.4; 1H NMR (DMSO-d6, 90° C., ppm): δ=1.0 (6H, d); 2.5 (4H, m); 2.7 (1H, m); 3.75 (4H, t); 4.9 (2H, q); 7.3 (1H, d); 7.5 (1H, t); 7.85 (1H, d); 8.2 (1H, s); 9.6 (1H, s).

### 47. N,N-dimethyl-4-[4-(2,2,2-trifluoroethoxy)-6-{[3-(trifluoromethyl)phenyl]amino}-1,3,5-triazin-2-yl]piperazine-1-carboxamide

LCMS: M+1=493.4; 1H NMR (DMSO-d6, 90° C., ppm): δ=2.8 (6H, s); 3.25 (4H, t); 3.8 (4H, t); 4.95 (2H, q); 7.35 (1H, d); 7.5 (1H, t); 7.85 (1H, d); 8.2 (1H, s); 9.6 (1H, s).

### 48. 4-[4-(methylsulfonyl)piperazin-1-yl]-6-(2,2,2-trifluoroethoxy)-N-[3-(trifluoromethyl)phenyl]-1,3,5-triazin-2-amine

LCMS: M+1=500.4; 1H NMR (DMSO-d6, 90° C., ppm): δ=2.5 (3H, m); 3.25 (4H, t); 3.9 (4H, t); 4.95 (2H, q); 7.35 (1H, d); 7.55 (1H, t); 7.85 (1H, d); 8.2 (1H, s); 9.55 (1H, s).

### 49. 2,2,2-trifluoro-1-{4-[4-(2,2,2-trifluoroethoxy)-6-{[3-(trifluoromethyl)phenyl]amino}-1,3,5-triazin-2-yl]piperazin-1-yl}ethanone

LCMS: M+1=518.3; 1H NMR (DMSO-d6, 90° C., ppm): δ=3.8 (8H, m); 4.95 (2H, q); 7.3 (1H, d); 7.55 (1H, t); 7.85 (1H, d); 8.15 (1H, s); 9.8 (1H, s).

### 50. N-phenyl-4-[4-(2,2,2-trifluoroethoxy)-6-{[3-(trifluoromethyl)phenyl]amino}-1,3,5-triazin-2-yl]piperazine-1-carboxamide

LCMS: M+1=464.3; 1H NMR (DMSO-d6, 90° C., ppm): δ=2.0 (3H, s); 3.6 (4H, t); 3.8 (4H, t); 4.9 (2H, q); 7.35 (1H, d); 7.5 (1H, t); 7.85 (1H, d); 8.2 (1H, s); 9.7 (1H, s).

### 51. N-(4-chlorophenyl)-4-[4-(2,2,2-trifluoroethoxy)-6-{[3-(trifluoromethyl)phenyl]amino}-1,3,5-triazin-2-yl]piperazine-1-carboxamide

LCMS: M+1=464.4; 1H NMR (DMSO-d6, 90° C., ppm): δ=1.0 (6H, d); 2.5 (4H, m); 2.7 (1H, m); 3.75 (4H, t); 4.9 (2H, q); 7.3 (1H, d); 7.5 (1H, t); 7.85 (1H, d); 8.2 (1H, s); 9.6 (1H, s).

### 52. methyl N-{[4-(4-(2,2,2-trifluoroethoxy)-6-{[3-(trifluoromethyl)phenyl]amino}-1,3,5-triazin-2-yl)piperazin-1-yl]carbonyl}glycinate

1H NMR (DMSO-d6, 90° C., ppm): δ=2.48 t (4H, 2CH2), 3.58 s (2H, CH2), 3.81 t (4H, 2CH2), 4.94 q (2H, CH2), 7.04 td (1H, Ar), 7.16 t (2H, Ar), 7.25-7.41 m (2H, Ar), 7.51 t (1H, Ar), 7.84 d (1H, Ar), 8.20 s (1H, Ar), 9.66 s (1H, NH). LC-MS [M+1]: calc'd: 531.5; obs'd: 531.5.

### 53. 4-(4-benzylpiperazin-1-yl)-6-(2,2,2-trifluoroethoxy)-N-[3-(trifluoromethyl)phenyl]-1,3,5-triazin-2-amine

1H NMR (DMSO-d6, 90° C., ppm): d=2.48 t (4H, 2CH2), 3.55 s (2H, CH2), 3.80 t (4H, 2CH2), 4.94 q (2H, CH2), 7.21-7.37 m (6H, Ar), 7.51 t (1H, Ar), 7.83 d (1H, Ar), 8.20 s (1H, Ar), 9.66 s (1H, NH). LC-MS [M+1]: calc'd: 513.5; obs'd: 513.6.

### 54. 4-[4-(3-fluorobenzyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=2.48 t (4H, 2CH2), 3.58 s (2H, CH2), 3.81 t (4H, 2CH2), 4.94 q (2H, CH2), 7.04 td (1H, Ar), 7.16 t (2H, Ar), 7.25-7.41 m (2H, Ar), 7.51 t (1H, Ar), 7.84 d (1H, Ar), 8.20 s (1H, Ar), 9.66 s (1H, NH). LC-MS [M+1]: calc'd: 531.5; obs'd: 531.5.

### 55. 4-[4-(4-fluorobenzyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=1.25 t (4H, 2CH2), 3.04 m (4H, 2CH2), 3.00-3.14 m (2H, CH2), 4.05 s br. (4H, 2CH2), 4.12 s (2H, CH2), 4.97 q (2H, CH2), 7.21 t (2H, Ar), 7.34 d (1H, Ar), 7.48-7.72 m (3H, Ar), 7.87 d (1H, Ar), 8.15 s (1H, Ar), 9.80 s (1H, NH). LC-MS [M+1]: calc'd: 531.5; obs'd: 531.5.

### 56. 4-{-4-[4-(dimethylamino)benzyl]piperazin-1-yl}-6-(2,2,2-trifluoroethoxy)-N-[3-(trifluoromethyl)phenyl]-1,3,5-triazin-2-amine

1H NMR (DMSO-d6, 90° C., ppm): d=2.44 t (4H, 2CH2), 2.88 s (6H, 2CH3), 3.43 (2H, CH2), 3.78 t (4H, 2CH2), 4.94 q (2H, CH2), 6.70 d (2H, Ar), 7.13 d (2H, Ar), 7.31 d (1H, Ar), 7.50 t (1H, Ar), 7.83 d (1H, Ar), 8.20 s (1H, Ar), 9.65 s (1H, NH). LC-MS [M+1]: calc'd: 556.5; obs'd: 556.5 (decomp'd: 134.3).

### 57. 4-[4-(3,4-dimethylbenzyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=2.22 d (6H, 2CH3), 2.45 t (4H, 2CH2), 3.46 (2H, CH2), 3.78 t (4H, 2CH2), 4.95 q (2H, CH2), 6.96-7.13 m (3H, Ar), 7.31 d (1H, Ar), 7.51 t (1H, Ar), 7.83 d (1H, Ar), 8.21 s (1H, Ar), 9.72 s (1H, NH). LC-MS [M+1]: calc'd: 541.5; obs'd: 541.5.

### 58. 4-[4-(2-fluorobenzyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=2.48 t (4H, 2CH2), 3.63 s (2H, CH2), 3.80 t (4H, 2CH2), 4.94 q (2H, CH2), 7.08-7.23 m (2H, Ar), 7.26-7.36 m (2H, Ar), 7.40-7.56 m (2H, Ar), 7.84 d (1H, Ar), 8.20 s (1H, Ar), 9.67 s (1H, NH). LC-MS [M+1]: calc'd: 531.5; obs'd: 531.4.

### 59. 4-(piperazin-1-yl)-6-(2,2,2-trifluoroethoxy)-N-[3-(trifluoromethyl)phenyl]-1,3,5-triazin-2-amine

LCMS: M+1=422.3; 1H NMR (DMSO-d6, 90° C., ppm): 1=2.88 m (4H), 3.38 m (1H), 3.79 m (4H), 4.99 m (2H), 7.52 t (1H), 7.61 dd (2H), 8.30 s (1H), 10.14 s (1H).

### 60. 4-[4-(2-methylbenzyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=2.40 s (3H, CH3), 3.00 m (4H, 2CH2), 3.93 m (4H, 2CH2), 4.03 s (2H, CH2), 4.98 q (2H, CH2), 7.17-7.31 m (3H, Ar), 7.34 d (1H, Ar), 7.41 d (1H, Ar), 7.53 t (1H, Ar), 7.86 d (1H, Ar), 8.17 s (1H, Ar), 9.82 s (1H, NH); LC-MS [M+1]: calc'd: 527.5; obs'd: 527.4.

### 61. 4-[4-(3-methylbenzyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=2.36 s (3H, CH3), 3.13 m (4H, 2CH2), 3.99 m (4H, 2CH2), 4.17 s (2H, CH2), 4.98 q (2H, CH2), 7.20-7.40 m (5H, Ar), 7.54 t (1H, Ar), 7.86 d (1H, Ar), 8.15 s (1H, Ar), 9.86 s (1H, NH); LC-MS [M+1]: calc'd: 527.5; obs'd: 527.4.

### 62. 4-[4-(4-methylbenzyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=2.30 s (3H, CH3), 2.46 t (4H, 2CH2), 3.51 s (2H, CH2), 3.79 t (4H, 2CH2), 4.95 q (2H, CH2), 7.14 d (2H, Ar), 7.22 d (2H, Ar), 7.32 d (1H, Ar), 7.51 t (1H, Ar), 7.83 d (1H, Ar), 8.21 s (1H, Ar), 9.73 s (1H, NH). LC-MS [M+1]: calc'd: 527.5; obs'd: 527.5.

### 63. 4-[4-(4-propylbenzyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=0.91 t (3H, CH3), 1.62 m (2H, CH2), 2.46 t (4H, 2CH2), 2.56 t (2H, CH2), 3.51 s (2H, CH2), 3.79 t (4H, 2CH2), 4.95 q (2H, CH2), 7.14 d (2H, Ar), 7.23 d (2H, Ar), 7.31 d (1H, Ar), 7.51 t (1H, Ar), 7.84 d (1H, Ar), 8.21 s (1H, Ar), 9.72 s (1H, NH). LC-MS [M+1]: calc'd: 555.5; obs'd: 555.3.

### 64. 4-[4-(pyridin-2-ylmethyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=2.29 m (6H, 2CH3), 2.49 m (4H, 2CH2), 3.47 s (2H, CH2), 3.78 t (4H, 2CH2), 4.95 q (2H, CH2), 6.92-7.12 m (3H, Ar), 7.31 d (1H, Ar), 7.51 t (1H, Ar), 7.85 d (1H, Ar), 8.21 s (1H, Ar), 9.66 s (1H, NH). LC-MS [M+1]: calc'd: 541.5; obs'd: 541.5.

### 65. 4-[4-(pyridin-4-ylmethyl)piperazin-1-yl]-6-(2,2,2-trifluoroethoxy)-N-[3-(trifluoromethyl)phenyl]-1,3,5-triazin-2-amine

1H NMR (DMSO-d6, 90° C., ppm): d=2.50 m (4H, 2CH2), 3.59 s (2H, CH2), 3.82 m (4H, 2CH2), 4.96 q (2H, CH2), 7.26-7.39 m (3H, Ar), 7.52 t (1H, Ar), 7.84 d (1H, Ar), 8.52 d (2H, Ar), 9.74 s (1H, NH); LC-MS [M+1]: calc'd: 514.5; obs'd: 514.6.

### 66. 4-{4-[(6-methylpyridin-2-yl)methyl]piperazin-1-yl}-6-(2,2,2-trifluoroethoxy)-N-[3-(trifluoromethyl)phenyl]-1,3,5-triazin-2-amine

1H NMR (DMSO-d6, 90° C., ppm): d=2.46 s (3H, CH3), 2.56 t (4H, 2CH2), 3.64 s (2H, CH2), 3.82 t (4H, 2CH2), 4.95 q (2H, CH2), 7.11 d (1H, Ar), 7.21-7.37 m (2H, Ar), 7.52 t (1H, Ar), 7.64 t (1H, Ar), 7.84 d (1H, Ar), 8.21 s (1H, Ar), 9.68 s (1H, NH); LC-MS [M+1]: calc'd: 528.5; obs'd: 528.7.

### 67. 4-(4-((tetrahydrofuran-2-yl)methyl)piperazin-1-yl)-6-(2,2,2-trifluoroethoxy)-N-(3-(trifluoromethyl)phenyl)-1,3,5-triazin-2-amine

1H NMR (DMSO-d6, 90° C., ppm): d=1.45-1.63 m (1H, CH2), 1.72-1.88 m (2H, CH2), 1.89-2.02 m (1H, CH2), 2.47 m (2H, CH2), 2.58 m (2H, CH2), 3.63 q (1H, CH2), 3.71-3.82 m (5H, 2.5CH2), 3.98 m (1H, CH), 4.95 q (2H, CH2), 7.32 d (1H, Ar), 7.52 t (1H, Ar), 7.84 d (1H, Ar), 8.22 s (1H, Ar), 9.67 s (1H, NH). LC-MS [M+1]: calc'd: 507.5; obs'd: 507.7.

### 68. methyl 5-({4-[4-(2,2,2-trifluoroethoxy)-6-{[3-(trifluoromethyl)phenyl]amino}-1,3,5-triazin-2-yl]piperazin-1-yl}methyl)furan-2-carboxylate

1H NMR (DMSO-d6, 90° C., ppm): d=2.54 m (4H, 2CH2), 3.68 s (2H, CH2), 3.79 m (4H, 2CH2), 3.81 s (3H, CH3), 4.96 q (2H, CH2), 6.53 d (1H, Ar), 7.21 d (1H, Ar), 7.33 d (1H, Ar), 7.52 t (1H, Ar), 7.84 d (1H, Ar), 8.21 s (1H, Ar), 9.73 s (1H, NH); LC-MS [M+1]: calc'd: 561.5; obs'd: 561.5.

### 69. 4-{4-[(3,5-dimethylisoxazol-4-yl)methyl]piperazin-1-yl}-6-(2,2,2-trifluoroethoxy)-N-[3-(trifluoromethyl)phenyl]-1,3,5-triazin-2-amine

1H NMR (DMSO-d6, 90° C., ppm): d=2.21 s (3H, CH3), 2.34 s (3H, CH3), 2.44 m (4H, 2CH2), 3.32 s (2H, CH2), 3.78 m (4H, 2CH2), 4.96 q (2H, CH2), 7.33 d (1H, Ar), 7.52 t (1H, Ar), 7.84 d (1H, Ar), 8.22 s (1H, Ar), 9.74 s (1H, NH); LC-MS [M+1]: calc'd: 532.5; obs'd: 532.4.

### 70. 4-{4-[4-(dimethylamino)benzoyl]piperazin-1-yl}-6-(2,2,2-trifluoroethoxy)-N-[3-(trifluoromethyl)phenyl]-1,3,5-triazin-2-amine

1H NMR (DMSO-d6, 90° C., ppm): d=2.97 s (6H, 2CH3), 3.63 m (4H, 2CH2), 3.85 t (4H, 2CH2), 4.97 q (2H, CH2), 6.75 d (2H, Ar), 7.34 d (3H, Ar), 7.53 t (1H, Ar), 7.88 d (1H, Ar), 8.19 s (1H, Ar), 9.73 s (1H, NH). LC-MS [M+1]: calc'd: 570.5; obs'd: 570.9.

### Generic Synthesis of Intermediates and Final Compounds for Table 35.

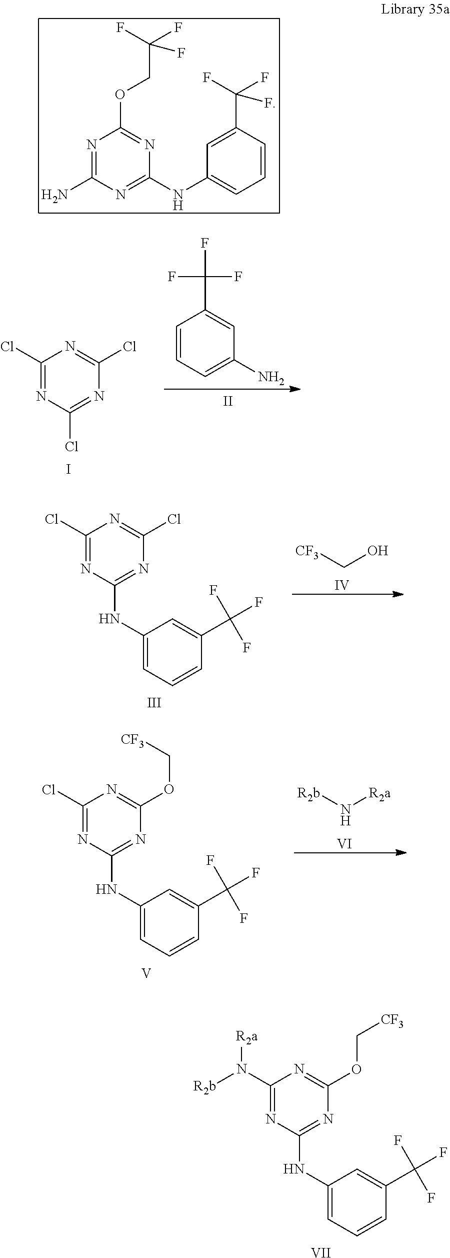

Preparation of 4-chloro-6-trifluoroethoxy-N-[3-(trifluoromethyl)phenyl]-1,3,5-triazine-2-amine V. Step 1: To a suspension of 0.500 g (2.78 s1 mmol) I, 0.225 g (2.781 mmol) of NaHCO₃ and 0.820 g (5.788 mmol) of Na₂SO₄ in 20 ml of anhydrous acetonitrile at −10° C. was added dropwise a solution of 3-(trifluoromethyl)aniline II, 0.450 g (2.781 mmol), in 10 ml of dry acetonitrile, over 2 h. After the addition was complete, the cooling bath was removed and the mixture was stirred at rt for 3 h. The resulting precipitate was filtered and the pale yellow solution of compound III (88% LCMS) was used in the next step without further purification.

Step 2: To a solution of compound III in 30 ml anhydrous acetonitrile was added dropwise a cooled solution of potassium tert-butoxide (0.312 g, 2.781 mmol) in 2,2,2-trifluoro-1-ethanol (5 ml) over 2 h. After stirring this reaction mixture overnight at rt, the solid precipitate was filtered and washed with anhydrous acetonitrile (2×30 ml). The solvent was removed in vacuo to afford a yellow oil. To this oil was added (3×50 ml) anhydrous hexanes and the mixture was heated at reflux. After 1 min the hexane layer was decanted. The solvent was removed in vacuo to afford Va as a white solid (0.595 g, 60%).

Preparation of 6-trifluoroethoxy-N-[3-(trifluoromethyl)phenyl]-1,3,5-triazine-2,4-diamines VII. 4-chloro-6-ethoxy-N-[3-(trifluoromethyl)phenyl]-1,3,5-triazin-2-amine V (0.040 g, 0.125 mmol) was suspended in dioxane (5 ml) in the presence of K₂CO₃ (0.035 g, 0.251 mmol). This mixture was treated with appropriate amines (0.125 mmol) at rt. The reaction mixture was then stirred for 30-40 min at 70-80° C. The reaction mixture was cooled, poured into water and extracted with CH₂Cl₂ or alternatively, CHCl₃. The organic extract was separated, dried over MgSO₄, filtered and concentrated. The residue was filtered, washed with hot hexanes, then with water and dried in vacuo (method a). Noncrystalline products were purified by silica gel chromatography with appropriate eluents (hexanes-ethyl acetate or methylene chloride-ethanol) (method b).

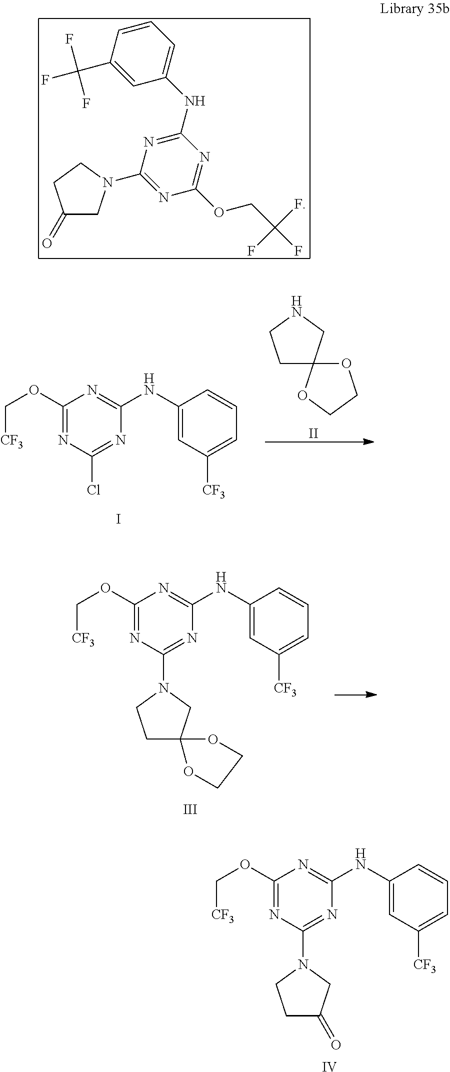

Preparation of 4-(1,4-dioxa-7-azaspiro[4.4]non-7-yl)-6-(2,2,2-trifluoroethoxy)-N-[3-(trifluoro-methyl)phenyl]-1,3,5-triazin-2-amine (III). 2-Chloro-4-trifluoroethoxy-6-(N-3-trifluoromethylanilino)-1,3,5-triazine I (0.5 mmol) was dissolved in 1,4-dioxane (5 ml) and treated with K₂CO₃ (0.75 mmol). 1,4-dioxa-7-azaspiro[4.4]nonane II (0.55 mmol) was added to this solution and the reaction mixture was heated to 80° C. LCMS analysis of reaction mixtures after stirring for 12 h demonstrated that the main product of this reaction was intended compound III. The reaction mixture was diluted with water (500 ml) and the resultant solid filtered, washed with water and then with hexanes. Crude material was crystallized from ethanol and then lyophilized (1.1 g, 76%). LCMS: M+1=466; NMR ¹H, DMSO, ppm: 1.88 m (4H, CH); 2.44 s (2H CH); 2.56 m (2H, CH); 3.64 s (2H, CH); 4.0 m (2H, CH); 6.54 d (2H, CH); 6.56 t (1H, CH); 7.2 d (1H, CH), 9.44 s (1H, NH).

Preparation of 1-(4-(2,2,2-trifluoroethoxy)-6-{[3-(trifluoromethyl)phenyl]amino}-1,3,5-triazin-2-yl)pyrrolidin-3-one (IV) 4-(1,4-dioxa-7-azaspiro[4.4]non-7-yl)-6-(2,2,2-trifluoroethoxy)-N-[3-(trifluoromethyl)phenyl]-1,3,5-triazin-2-amine III (0.5 g) was dissolved in 3 ml of THF and hydrolyzed with 11 ml of 3N HCl at rt for 72 h. The product was isolated by vacuum filtration (0.75 g, 78%). LCMS: M+1=422; NMR 1H, DMSO, ppm: 2.64 m (2H, CH); 4.02 m (4H CH); 4.88 m (2H, CH); 7.22 d (1H, CH); 7.45 t (1H, CH); 7.88 d (1H, CH), 8.28 s (1H, CH), 10.1 s (1H, NH)

TABLE 35

| En | Structure | IUPAC Name | MW | Formula |
|---|---|---|---|---|
| 1 | 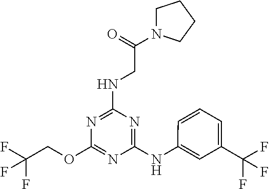 | 1-(pyrrolidin-1-yl)-2-{[4- (2,2,2-trifluoroethoxy)-6- {[3-(trifluoromethyl)phenyl] amino}-1,3,5-triazin-2- yl]amino}ethanone | 464.4 | C₁₈H₁₈F₆N₆O₂ |
| 2 | 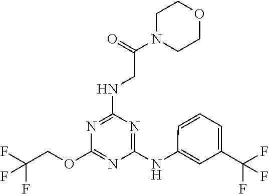 | 1-(morpholin-4-yl)-2-{[4- (2,2,2-trifluoroethoxy)-6- {[3-(trifluoromethyl)phenyl] amino}-1,3,5-triazin-2-yl] amino}ethanone | 480.4 | C₁₈H₁₈F₆N₆O₃ |
| 3 | 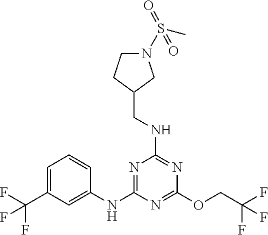 | N-{[1-(methylsulfonyl) pyrrolidin-3-yl]methyl}-6- (2,2,2-trifluoroethoxy)-N′- [3-(trifluoromethyl)phenyl]- 1,3,5-triazine-2,4-diamine | 515.5 | C₁₈H₂₀F₆N₆O₃S |
| 4 | 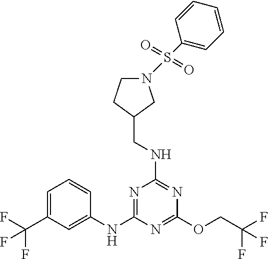 | N-{[1-(phenylsulfonyl) pyrrolidin-3-yl]methyl}- 6-(2,2,2-trifluoroethoxy)- N′-[3-(trifluoromethyl) phenyl]-1,3,5-triazine- 2,4-diamine | 577.5 | C₂₃H₂₂F₆N₆O₃S |
| 5 | 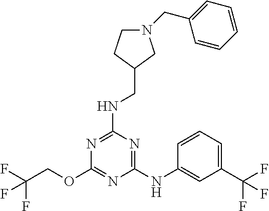 | N-[(1-benzylpyrrolidin-3- yl)methyl]-6-(2,2,2- trifluoroethoxy)-N′-[3- (trifluoromethyl)phenyl]- 1,3,5-triazine-2,4-diamine | 526.5 | C₂₄H₂₄F₆N₆O |
| 6 | 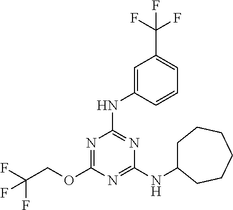 | N-cycloheptyl-6-(2,2,2- trifluoroethoxy)-N′- [3-(trifluoromethyl)phenyl]- 1,3,5-triazine-2,4-diamine | 449.4 | C₁₉H₂₁F₆N₅O |
| 7 | 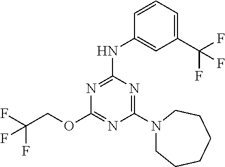 | 4-(azepan-1-yl)-6-(2,2,2- trifluoroethoxy)-N-[3- (trifluoromethyl)phenyl]- 1,3,5-triazin-2-amine | 435.4 | C₁₈H₁₉F₆N₅O |
| 8 | 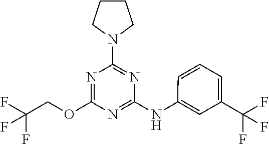 | 4-(pyrrolidin-1-yl)-6- (2,2,2-trifluoroethoxy)- N-[3-(trifluoromethyl) phenyl]-1,3,5-triazin- 2-amine | 407.3 | C₁₆H₁₅F₆N₅O |
| 9 | 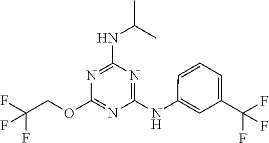 | N-(propan-2-yl)-6-(2,2,2- trifluoroethoxy)-N′-[3- (trifluoromethyl)phenyl]- 1,3,5-triazine-2,4-diamine | 395.3 | C₁₅H₁₅F₆N₅O |
| 10 | 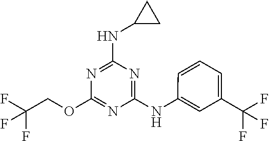 | N-cyclopropyl-6-(2,2,2- trifluoroethoxy)-N′-[3- (trifluoromethyl)phenyl]- 1,3,5-triazine-2,4-diamine | 393.3 | C₁₅H₁₃F₆N₅O |
| 11 | 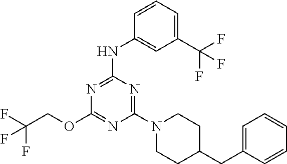 | 4-(4-benzylpiperidin-1-yl)- 6-(2,2,2-trifluoroethoxy)-N- [3-(trifluoromethyl)phenyl]- 1,3,5-triazin-2-amine | 511.5 | C₂₄H₂₃F₆N₅O |
| 12 | 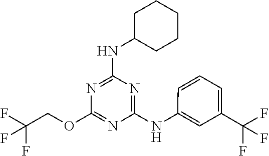 | N-cyclohexyl-6-(2,2,2- trifluoroethoxy)-N′-[3- (trifluoromethyl)phenyl]- 1,3,5-triazine-2,4-diamine | 435.4 | C₁₈H₁₉F₆N₅O |
| 13 | 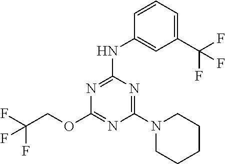 | 4-(piperidin-1-yl)-6- (2,2,2-trifluoroethoxy)- N-[3-(trifluoromethyl) phenyl]-1,3,5-triazin- 2-amine | 421.3 | C₁₇H₁₇F₆N₅O |
| 14 | 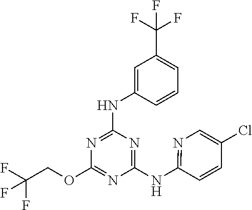 | N-(5-chloropyridin-2-yl)- 6-(2,2,2-trifluoroethoxy)- N′-[3-(trifluoromethyl) phenyl]-1,3,5-triazine- 2,4-diamine | 464.7 | C₁₇H₁₁ClF₆N₆O |
| 15 | 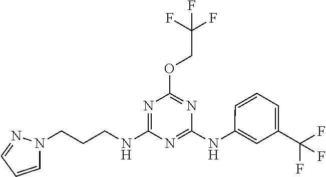 | N-[3-(1H-pyrazol-1- yl)propyl]-6-(2,2,2- trifluoroethoxy)-N′-[3- (trifluoromethyl)phenyl]- 1,3,5-triazine-2,4-diamine | 461.4 | C₁₈H₁₇F₆N₇O |
| 16 | 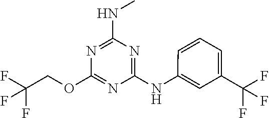 | N-methyl-6-(2,2,2- trifluoroethoxy)-N′-[3- (trifluoromethyl)phenyl]- 1,3,5-triazine-2,4-diamine | 367.3 | C₁₃H₁₁F₆N₅O |
| 17 | 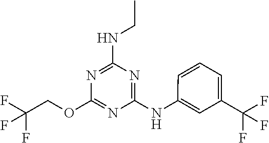 | N-ethyl-6-(2,2,2- trifluoroethoxy)-N′-[3- (trifluoromethyl)phenyl]- 1,3,5-triazine-2,4-diamine | 381.3 | C₁₄H₁₃F₆N₅O |
| 18 | 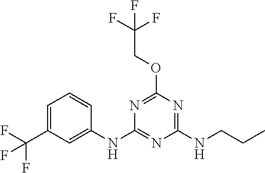 | N-propyl-6-(2,2,2- trifluoroethoxy)-N′-[3- (trifluoromethyl)phenyl]- 1,3,5-triazine-2,4-diamine | 395.3 | C₁₅H₁₅F₆N₅O |
| 19 | 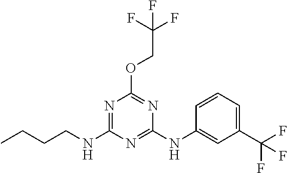 | N-butyl-6-(2,2,2- trifluoroethoxy)-N′-[3- (trifluoromethyl)phenyl]- 1,3,5-triazine-2,4-diamine | 409.3 | C₁₆H₁₇F₆N₅O |
| 20 | 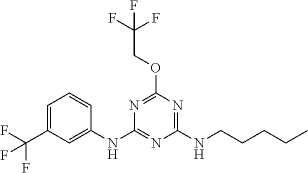 | N-pentyl-6-(2,2,2- trifluoroethoxy)-N′- [3-(trifluoromethyl) phenyl]-1,3,5- triazine-2,4-diamine | 423.4 | C₁₇H₁₉F₆N₅O |
| 21 | 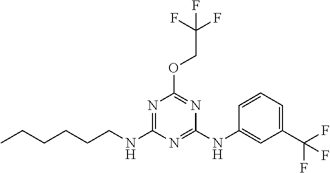 | N-hexyl-6-(2,2,2- trifluoroethoxy)-N′-[3- (trifluoromethyl)phenyl]- 1,3,5-triazine-2,4-diamine | 437.4 | C₁₈H₂₁F₆N₅O |
| 22 | 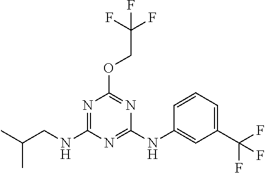 | N-(2-methylpropyl)-6- (2,2,2-trifluoroethoxy)- N′-[3-(trifluoromethyl) phenyl]-1,3,5-triazine- 2,4-diamine | 409.3 | C₁₆H₁₇F₆N₅O |
| 23 | 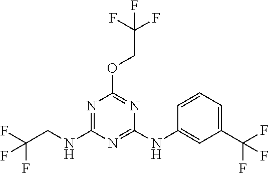 | 6-(2,2,2-trifluoroethoxy)- N-(2,2,2- trifluoroethyl)-N′-[3- (trifluoromethyl)phenyl]- 1,3,5-triazine-2,4-diamine | 435.2 | C₁₄H₁₀F₉N₅O |
| 24 | 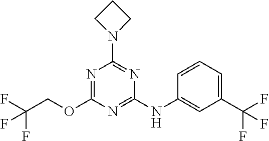 | 4-(azetidin-1-yl)-6-(2,2,2- trifluoroethoxy)-N-[3- (trifluoromethyl)phenyl]- 1,3,5-triazin-2-amine | 393.3 | C₁₅H₁₃F₆N₅O |
| 25 | 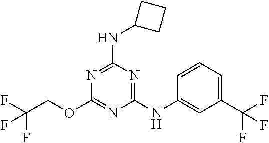 | N-cyclobutyl-6-(2,2,2- trifluoroethoxy)-N′-[3- (trifluoromethyl)phenyl]- 1,3,5-triazine-2,4-diamine | 407.3 | C₁₆H₁₅F₆N₅O |
| 26 | 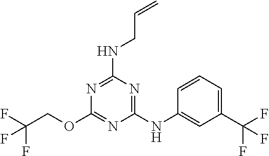 | N-(prop-2-en-1-yl)-6- (2,2,2-trifluoroethoxy)-N′- [3-(trifluoromethyl)phenyl]- 1,3,5-triazine-2,4-diamine | 393.3 | C₁₅H₁₃F₆N₅O |
| 27 | 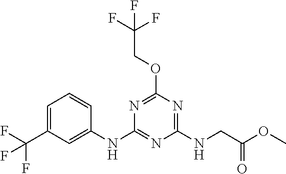 | methyl N-[4-(2,2,2- trifluoroethoxy)-6-{[3- (trifluoromethyl)phenyl] amino}-1,3,5-triazin- 2-yl]glycinate | 425.3 | C₁₅H₁₃F₆N₅O₃ |
| 28 | 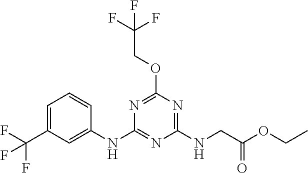 | ethyl N-[4-(2,2,2- trifluoroethoxy)-6- {[3-(trifluoromethyl) phenyl]amino}-1,3,5- triazin-2-yl]glycinate | 439.3 | C₁₆H₁₅F₆N₅O₃ |
| 29 | 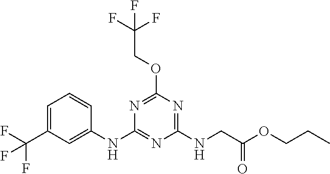 | propyl N-[4-(2,2,2- trifluoroethoxy)-6-{[3- (trifluoromethyl)phenyl] amino}-1,3,5-triazin- 2-yl]glycinate | 453.3 | C₁₇H₁₇F₆N₅O₃ |
| 30 | 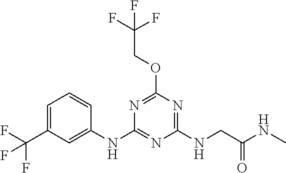 | N-methyl-N-2-[4-(2,2,2- trifluoroethoxy)-6-{[3- (trifluoromethyl)phenyl] amino}-1,3,5-triazin-2- yl]glycinamide | 424.4 | C₁₅H₁₄F₆N₆O₂ |
| 31 | 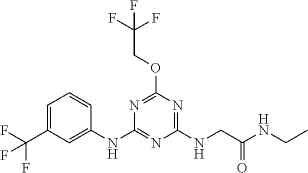 | N-ethyl-N-2-[4-(2,2,2- trifluoroethoxy)-6-{[3- (trifluoromethyl)phenyl] amino}-1,3,5-triazin- 2-yl]glycinamide | 438.3 | C₁₆H₁₆F₆N₆O₂ |
| 32 | 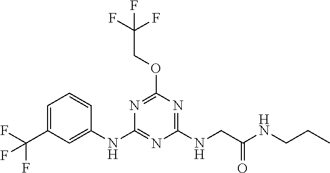 | N-propyl-N-2-[4-(2,2,2- trifluoroethoxy)-6-{[3- (trifluoromethyl)phenyl] amino}-1,3,5-triazin- 2-yl]glycinamide | 452.4 | C₁₇H₁₈F₆N₆O₂ |
| 33 | 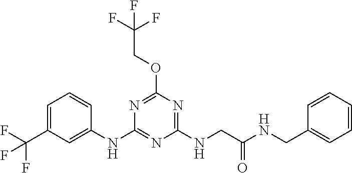 | N-benzyl-N-2-[4-(2,2,2- trifluoroethoxy)-6-{[3- (trifluoromethyl)phenyl] amino}-1,3,5-triazin- 2-yl]glycinamide | 500.4 | C₂₁H₁₈F₆N₆O₂ |
| 34 | 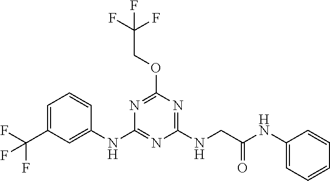 | N-phenyl-N-2-[4-(2,2,2- trifluoroethoxy)-6-{[3- (trifluoromethyl)phenyl] amino}-1,3,5-triazin- 2-yl]glycinamide | 486.4 | C₂₀H₁₆F₆N₆O₂ |
| 35 | 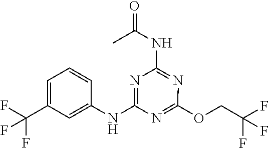 | N-[4-(2,2,2-trifluoroethoxy)- 6-{[3-(trifluoromethyl) phenyl]amino}-1,3,5- triazin-2-yl]acetamide | 395.3 | C₁₄H₁₁F₆N₅O₂ |
| 36 | 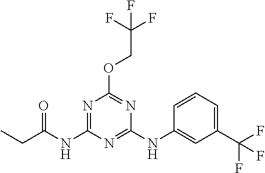 | N-[4-(2,2,2-trifluoroethoxy)- 6-{[3-(trifluoromethyl) phenyl]amino}-1,3,5- triazin-2-yl]propanamide | 409.3 | C₁₅H₁₃F₆N₅O₂ |
| 37 | 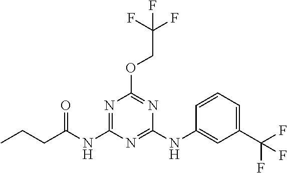 | N-[4-(2,2,2-trifluoroethoxy)- 6-{[3-(trifluoromethyl) phenyl]amino}-1,3,5- triazin-2-yl]butanamide | 423.3 | C₁₆H₁₅F₆N₅O₂ |
| 38 | 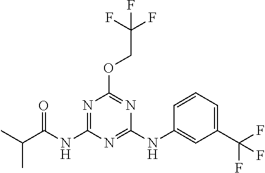 | 2-methyl-N-[4-(2,2,2- trifluoroethoxy)-6-{[3- (trifluoromethyl)phenyl] amino}-1,3,5-triazin-2- yl]propanamide | 423.3 | C₁₆H₁₅F₆N₅O₂ |
| 39 | 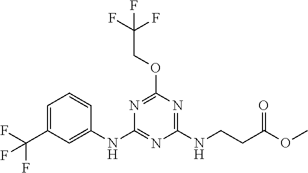 | methyl N-[4-(2,2,2- trifluoroethoxy)-6-{[3- (trifluoromethyl)phenyl] amino}-1,3,5-triazin- 2-yl]-beta-alaninate | 439.3 | C₁₆H₁₅F₆N₅O₃ |
| 40 | 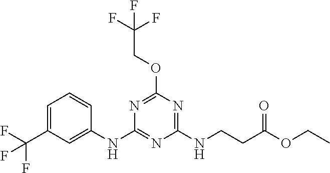 | ethyl N-[4-(2,2,2-trifluoro- ethoxy)-6-{[3-(trifluoro- methyl)phenyl]amino}- 1,3,5-triazin-2-yl]-beta- alaninate | 453.3 | C₁₇H₁₇F₆N₅O₃ |
| 41 | 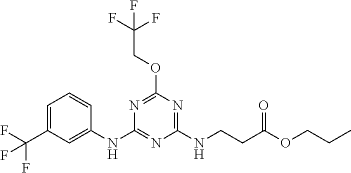 | propyl N-[4-(2,2,2-tri- fluoroethoxy)-6-{[3- (trifluoromethyl)phenyl] amino}-1,3,5-triazin- 2-yl]-beta-alaninate | 467.4 | C₁₈H₁₉F₆N₅O₃ |
| 42 | 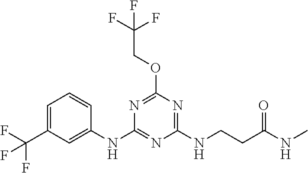 | N-methyl-N-3-[4-(2,2,2- trifluoroethoxy)-6-{[3- (trifluoromethyl)phenyl] amino}-1,3,5-triazin- 2-yl]-beta-alaninamide | 438.3 | C₁₆H₁₆F₆N₆O₂ |
| 43 | 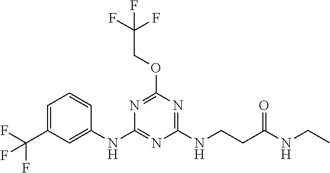 | N-ethyl-N-3-[4-(2,2,2- trifluoroethoxy)-6-{[3- (trifluoromethyl)phenyl] amino}-1,3,5-triazin- 2-yl]-beta-alaninamide | 452.4 | C₁₇H₁₈F₆N₆O₂ |
| 44 | 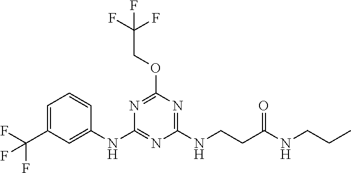 | N-propyl-N-3-[4-(2,2,2- trifluoroethoxy)-6-{[3- (trifluoromethyl)phenyl] amino}-1,3,5-triazin- 2-yl]-beta-alaninamide | 466.4 | C₁₈H₂₀F₆N₆O₂ |
| 45 | 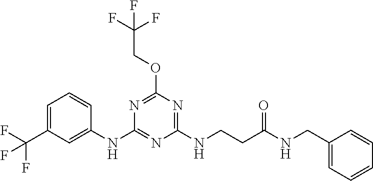 | N-benzyl-N-3-[4-(2,2,2- trifluoroethoxy)-6-{[3- (trifluoromethyl)phenyl] amino}-1,3,5-triazin- 2-yl]-beta-alaninamide | 514.4 | C₂₂H₂₀F₆N₆O₂ |
| 46 | 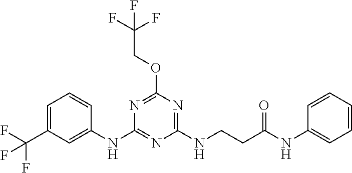 | N-phenyl-N-3-[4-(2,2,2- trifluoroethoxy)-6-{[3- (trifluoromethyl)phenyl] amino}-1,3,5-triazin-2- yl]-beta-alaninamide | 500.4 | C₂₁H₁₈F₆N₆O₂ |
| 47 | 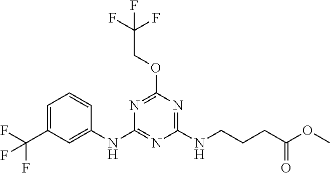 | methyl 4-{[4-(2,2,2- trifluoroethoxy)-6-{[3- (trifluoromethyl)phenyl] amino}-1,3,5-triazin- 2-yl]amino}butanoate | 453.3 | C₁₇H₁₇F₆N₅O₃ |
| 48 | 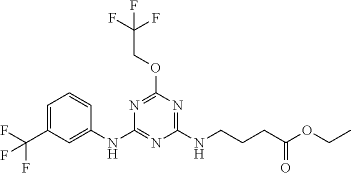 | ethyl 4-{[4-(2,2,2- trifluoroethoxy)-6-{[3- (trifluoromethyl)phenyl] amino}-1,3,5-triazin- 2-yl]amino}butanoate | 467.4 | C₁₈H₁₉F₆N₅O₃ |
| 49 | 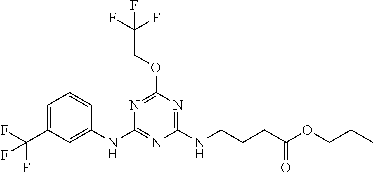 | propyl 4-{[4-(2,2,2- trifluoroethoxy)-6-{[3- (trifluoromethyl)phenyl] amino}-1,3,5-triazin- 2-yl]amino}butanoate | 481.4 | C₁₉H₂₁F₆N₅O₃ |
| 50 | 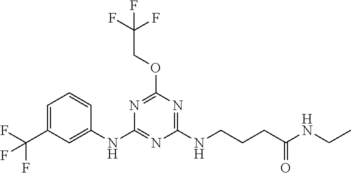 | N-ethyl-4-{[4-(2,2,2- trifluoroethoxy)-6-{[3- (trifluoromethyl)phenyl] amino}-1,3,5-triazin- 2-yl]amino}butanamide | 466.4 | C₁₈H₂₀F₆N₆O₂ |
| 51 | 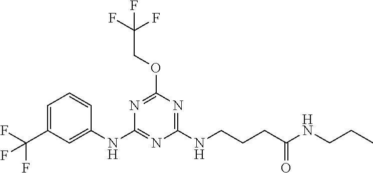 | N-propyl-4-{[4-(2,2,2- trifluoroethoxy)-6-{[3- (trifluoromethyl)phenyl] amino}-1,3,5-triazin- 2-yl]amino}butanamide | 480.4 | C₁₉H₂₂F₆N₆O₂ |
| 52 | 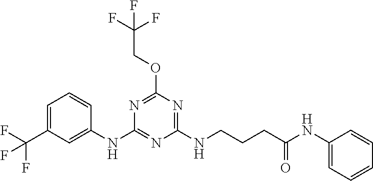 | N-phenyl-4-{[4-(2,2,2- trifluoroethoxy)-6-{[3- (trifluoromethyl)phenyl] amino}-1,3,5-triazin- 2-yl]amino}butanamide | 514.4 | C₂₂H₂₀F₆N₆O₂ |
| 53 | 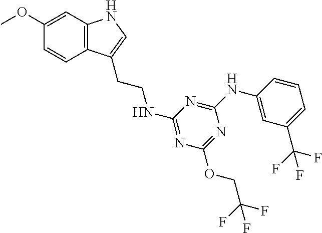 | N-[2-(6-methoxy- 1H-indol-3-yl)ethyl]- 6-(2,2,2-trifluoro- ethoxy)-N′-[3- (trifluoromethyl)phenyl]- 1,3,5-triazine-2,4- diamine | 526.4 | C₂₃H₂₀F₆N₆O₂ |
| 54 | 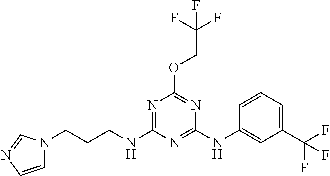 | N-[3-(1H-imidazol- 1-yl)propyl]-6-(2,2,2- trifluoroethoxy)-N′-[3- (trifluoromethyl)phenyl]- 1,3,5-triazine-2,4-diamine | 461.4 | C₁₈H₁₇F₆N₇O |
| 55 | 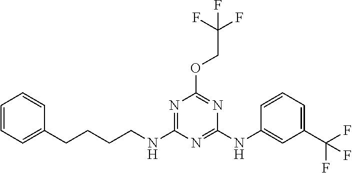 | N-(4-phenylbutyl)-6- (2,2,2-trifluoroethoxy)- N′-[3-(trifluoromethyl) phenyl]-1,3,5-triazine- 2,4-diamine | 485.4 | C₂₂H₂₁F₆N₅O |
| 56 | 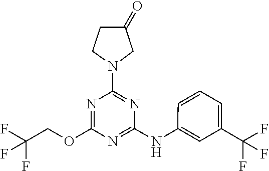 | 1-[4-(2,2,2-trifluoro- ethoxy)-6-{[3-(trifluoro- methyl)phenyl]amino}- 1,3,5-triazin-2- yl]pyrrolidin-3-one | 421.2 | C₁₆H₁₃F₆N₅O₂ |
| 57 | 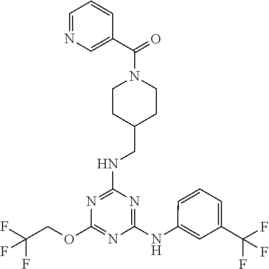 | pyridin-3-yl[4-({[4-(2,2,2- trifluoroethoxy)-6-{[3- (trifluoromethyl)phenyl] amino}-1,3,5- triazin-2-yl]amino} methyl)piperidin-1- yl]methanone | 555.5 | C₂₄H₂₃F₆N₇O₂ |
| 58 | 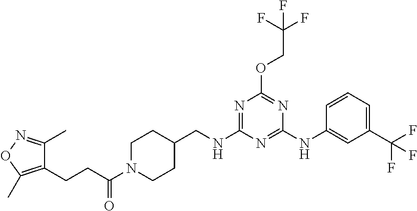 | 3-(3,5-dimethylisoxazol- 4-yl)-1-[4-({[4-(2,2,2- trifluoroethoxy)-6-{[3- (trifluoromethyl)phenyl] amino}-1,3,5-triazin- 2-yl]amino}methyl) piperidin-1-yl]propan- 1-one | 601.5 | C₂₆H₂₉F₆N₇O₃ |
| 59 | 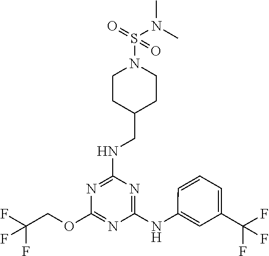 | N,N-dimethyl-4-({[4- (2,2,2-trifluoroethoxy)- 6-{[3-(trifluoromethyl) phenyl]amino}-1,3,5- triazin-2-yl]amino} methyl)piperidine-1- sulfonamide | 558.5 | C₂₀H₂₅F₆N₇O₃S |
| 60 | 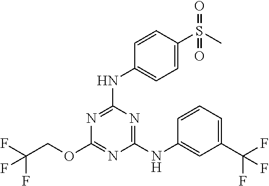 | N-[4-(methylsulfonyl) phenyl]-6-(2,2,2- trifluoroethoxy)-N′- [3-(trifluoromethyl) phenyl]-1,3,5- triazine-2,4-diamine | 508.4 | C₁₉H₁₅F₆N₅O₃S |
| 61 | 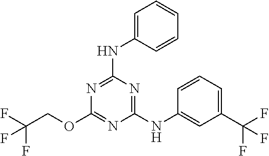 | N-phenyl-6-(2,2,2- trifluoroethoxy)-N′- [3-(trifluoromethyl) phenyl]-1,3,5-triazine- 2,4-diamine | 429.3 | C18H13F6N5O |
| 62 | 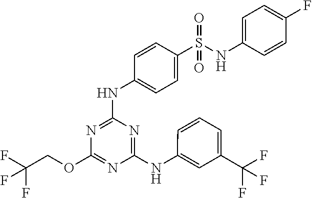 | N-(4-fluorophenyl)-4- [(4-(2,2,2-trifluoroethoxy)- 6-{[3-(trifluoromethyl) phenyl]amino}-1,3,5- triazin-2-yl)amino] benzenesulfonamide | 603.4 | C₂₄H₁₇F₇N₆O₃S |
| 63 | 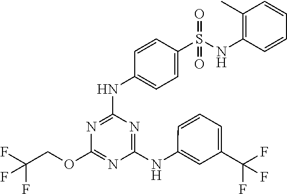 | N-(2-methylphenyl)-4- [(4-(2,2,2-trifluoroethoxy)- 6-{[3-(trifluoromethyl) phenyl]amino}-1,3,5- triazin-2-yl)amino] benzenesulfonamide | 599.5 3054 | C₂₅H₂₀F₆N₆O₃S |
| 64 | 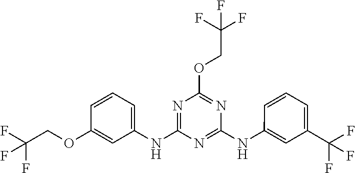 | 6-(2,2,2-trifluoroethoxy)- N-[3-(2,2,2- trifluoroethoxy)phenyl]- N′-[3-(trifluoromethyl) phenyl]-1,3,5-triazine- 2,4-diamine | 527.3 441 | C₂₀H₁₄F₉N₅O₂ |

### Procedures and Analytical Data for Table 35.

Entries 1 to 55, and 57 to 64 are Library 35a compounds. Entry 56 is a Library 35b compound.

### 1. 1-Pyrrolidin-1-yl-2-[4-(2,2,2-trifluoro-ethoxy)-6-(3-trifluoromethyl-phenylamino)-[1,3,5]-triazin-2-ylamino]-ethanone

Yield 200 mg, 86%. ¹H-NMR (400 MHz, DMSO-D₆) δ_{H}: 1.78 (2H, m), 1.93 (2H, m), 3.33 (2H, broad), 3.48 (2H, broad), 4.08 (2H, broad), 4.95 (2H, superposition of two broad q, J=7.5 Hz, Z/E forms), 7.32 (1H, d, J=8.5 Hz), 7.48 (2H, broad), 7.85-8.01 (1H, two broad d, J=8.5 Hz, Z/E forms), 8.13-8.16 (1H, two broad peaks, Z/E forms), 9.84-9.92 (1H, two broad peaks, Z/E forms). MW 464.37. LCMS t_{R} (min): 1.94. MS (APCI), m/z 465.12 [M+H]⁺. HPLC t_{R} (min): 14.15. M_{P} 226-227° C.

### 2. 1-Morpholin-4-yl-2-[4-(2,2,2-trifluoro-ethoxy)-6-(3-trifluoromethyl-phenylamino)-[1,3,5]-triazin-2-ylamino]-ethanone

Yield 200 mg, 83%. ¹H-NMR (400 MHz, DMSO-D₆) δ_{H}: 3.46 (4H, m), 3.59 (4H, m), 4.20 (2H, superposition of two d, J=7.5 Hz, Z/E forms), 4.95 (2H, broad q, J=7.5 Hz), 7.32 (1H, broad), 7.51 (1H, t, J=8.5 Hz), 7.56 (1H, broad), 7.87-8.02 (1H, two broad d, J=8.5 Hz, Z/E forms), 8.14 (1H, broad), 9.85-9.93 (1H, two broad peaks). MW 480.37. LCMS t_{R} (min): 1.92. MS (APCI), m/z 481.13 [M+H]⁺. HPLC t_{R} (min): 14.94. M_{P} 238-239° C.

### 3. N-(1-Methanesulfonyl-pyrrolidin-3-ylmethyl)-6-(2,2,2-trifluoro-ethoxy)-N′-(3-trifluoromethyl-phenyl)-[1,3,5]triazine-2,4-diamine (40b)

Yield 22 mg, 16%. ¹H-NMR (400 MHz, DMSO-D₆) δ_{H}: 1.66 (1H, m), 2.00 (1H, m), 2.55 (1H, m), 2.86 (3H, m) 3.00 (1H, m), 3.22 (1H, m), 3.31 (2H, m), 3.40 (2H, m), 4.97 (2H, superposition of two quartets, Z/E forms), 7.32 (1H, m), 7.50 (1H, m), 7.83 (1H, m), 7.95-8.05 (1H, broad, Z/E forms), 8.05-8.34 (1H, broad, Z/E forms), 9.77-9.95 (1H, broad, Z/E forms).

MW 514.45. LCMS t_{R} (min): 1.93. MS (APCI+), m/z 515.16 [M+H]⁺. HPLC t_{R} (min): 15.63. M_{P} 202-204° C.

### 4. N-(1-Benzenesulfonyl-pyrrolidin-3-ylmethyl)-6-(2,2,2-trifluoro-ethoxy)-N′-(3-trifluoromethyl-phenyl)-[1,3,5]triazine-2,4-diamine (40c)

Yield 120 mg, 39%. ¹H-NMR (400 MHz, DMSO-D₆) δ_{H}: 1.51 (1H, m), 1.91 (1H, m), 2.41 (1H, m), 2.91 (1H, m), 3.21 (5H, m), 4.92 (2H, q, J=7.5 Hz), 7.31 (1H, d, J=8.5 Hz), 7.52 (3H, m), 7.61 (1H, m), 7.72 (3H, m), 8.05 (1H, s), 8.28 (1H, broad peak, Z/E forms), 9.71-9.92 (1H, two broad peaks, Z/E forms).

MW 576.52. LCMS t_{R} (min): 2.12. MS (APCI+), m/z 576.99 [M+H]⁺. HPLC t_{R} (min): 17.41. M_{P} 240-242° C.

### 5. N-(1-Benzyl-pyrrolidin-3-ylmethyl)-6-(2,2,2-trifluoro-ethoxy)-N′-(3-trifluoro-methyl-phenyl)-[1,3,5]triazine-2,4-diamine

Yield 40 mg, 8%. ¹H-NMR (400 MHz, DMSO-D₆) δ_{H}: 1.45 (1H, broad peak), 1.91 (1H, broad peak), 2.31 (1H, broad peak), 3.02 (2H, m), 3.28 (2H, broad peak, Z/E forms), 3.61 (2H, broad peak, Z/E forms), 4.92 (2H, superposition of two broad q, J=7.5 Hz), 7.31 (5H, m), 7.51 (1H, t, J=8.5 Hz), 7.72-7.82 (1H, two broad peaks, Z/E forms), 7.82 (1H, broad peak, Z/E forms), 8.05 (1H, broad peak, Z/E forms), 8.42 (1H, broad peak, Z/E forms), 9.71-9.91 (1H, two broad peaks, Z/E forms).

MW 526.48. LCMS t_{R} (min): 1.72. MS (APCI+), m/z 527.19 [M+H]⁺. HPLC t_{R} (min): 13.99. M_{P} 167-168° C.

### 6. N-cycloheptyl-6-(2,2,2-trifluoroethoxy)-N′-[3-(trifluoromethyl)phenyl]-1,3,5-triazine-2,4-diamine

LCMS: M+1=450.5; 1H NMR, DMSO-d6 δ, ppm: 1.35-1.80 m (10H); 1.90 m (2H); 4.00 m (1H); 4.95 m (2H); 7.20-7.38 m (2H); 7.50 t (1H); 7.88-8.34 m (2H); 9.50 s (1H).

### 7. 4-(azepan-1-yl)-6-(2,2,2-trifluoroethoxy)-N-[3-(trifluoromethyl)phenyl]-1,3,5-triazin-2-amine

LCMS: M+1=435.3; 1H NMR (DMSO-d6, 90° C., ppm): d=1.54 t (4H), 1.78 q (4H), 3.76 m (4H), 4.96 q (2H), 7.30 d (1H), 7.50 t (1H), 7.82 d (1H), 8.34 d (1H), 9.62 s (1H).

### 8. 4-(pyrrolidin-1-yl)-6-(2,2,2-trifluoroethoxy)-N-[3-(trifluoromethyl)phenyl]-1,3,5-triazin-2-amine

LCMS: M+1=407.3; 1H NMR (DMSO-d6, 90° C., ppm): d=1.96 t (4H), 3.58 t (4H), 4.96 q (2H), 7.32 d (1H), 7.48 t (1H), 7.91 t (1H), 8.36 s (1H), 9.56 s (1H).

### 9. N-(propan-2-yl)-6-(2,2,2-trifluoroethoxy)-N′-[3-(trifluoromethyl)phenyl]-1,3,5-triazine-2,4-diamine

LCMS: M+1=395.3; 1H NMR (DMSO-d6, 90° C., ppm): d=1.24 d (6H), 4.20 m (1H), 4.92 q (2H), 7.28 m (2H), 7.48 t (1H), 7.92 s (1H), 8.28 s (1H), 9.52 s (1H).

### 10. N-cyclopropyl-6-(2,2,2-trifluoroethoxy)-N′-[3-(trifluoromethyl)phenyl]-1,3,5-triazine-2,4-diamine

LCMS: M+1=393.2; 1H NMR (DMSO-d6, 90° C., ppm): d=0.68 m (4H), 2.84 t (2H), 4.92 q (2H), 7.32 d (1H), 7.54 m (2H), 7.98 d (1H), 8.32 s (1H), 9.70 s (1H).

### 11. 4-(4-benzylpiperidin-1-yl)-6-(2,2,2-trifluoroethoxy)-N-[3-(trifluoromethyl)phenyl]-1,3,5-triazin-2-amine

LCMS: M+1=511.4; 1H NMR (DMSO-d6, 90° C., ppm): d=1.20 q (2H), 1.70 d (2H), 1.90 q (1H), 2.58 d (2H), 4.56 d (2H), 4.48 q (2H), 7.24 m (6H), 7.52 d (1H), 7.84 d (1H), 8.28 s (1H), 9.56 s (1H).

### 12. N-cyclohexyl-6-(2,2,2-trifluoroethoxy)-N′-[3-(trifluoromethyl)phenyl]-1,3,5-triazine-2,4-diamine

LCMS: M+1=435.3; 1H NMR (DMSO-d6, 90° C., ppm): d=1.60 m (11H), 3.02 m (1H), 3.86 s (1H), 4.92 q (2H), 7.36 m (3H), 8.12 t (2H), 9.52 s (1H).

### 13. 4-(piperidin-1-yl)-6-(2,2,2-trifluoroethoxy)-N-[3-(trifluoromethyl)phenyl]-1,3,5-triazin-2-amine

LCMS: M+1=421.3; 1H NMR (DMSO-d6, 90° C., ppm): d=1.64 d (6H), 3.80 t (4H), 4.92 q (2H), 7.32 d (1H), 7.50 t (1H), 7.68 d (1H), 8.28 s (1H), 9.64 s (1H).

### 14. N-(5-chloropyridin-2-yl)-6-(2,2,2-trifluoroethoxy)-N′-[3-(trifluoromethyl)phenyl]-1,3,5-triazine-2,4-diamine

LCMS: M+1=464.7; 5.0 (2H, m); 7.3 (1H, d); 7.6 (1H, t); 7.7 (1H, m); 8.2 (2H, m); 8.7 (1H, s); 9.9 (1H, d).

### 15. N-[3-(1H-pyrazol-1-yl)propyl]-6-(2,2,2-trifluoroethoxy)-N′-[3-(trifluoromethyl)phenyl]-1,3,5-triazine-2,4-diamine

LCMS: M+1=461.3; 2.1 (2H, m); 3.3 (2H, m); 4.2 (2H, t); 4.9 (2H, q); 6.2 (1H, m); 7.3 (1H, d); 7.5 (3H, m); 7.65 (1H, d); 7.9 (1H, d); 8.1 (1H, s); 9.6 (1H, s).

### 16. N-methyl-6-(2,2,2-trifluoroethoxy)-N′-[3-(trifluoromethyl)phenyl]-1,3,5-triazine-2,4-diamine

LCMS: M+1=367.2; 1H NMR (DMSO-d6, 90° C., ppm): 1=2.78 s (3H), 4.94 m (2H), 7.28 m (2H), 7.51 t (1H), 7.92 d (1H), 8.27 s (1H), 9.58 s (1H).

### 17. N-ethyl-6-(2,2,2-trifluoroethoxy)-N′-[3-(trifluoromethyl)phenyl]-1,3,5-triazine-2,4-diamine

LCMS: M+1=381.2; 1H NMR (DMSO-d6, 90° C., ppm): 1=1.09 t (3H), 3.42 s (2H), 4.98q (2H), 7.41 m (3H), 7.85 m (1H), 7.85 s (1H), 8.28 s (1H).

### 18. N-propyl-6-(2,2,2-trifluoroethoxy)-N′-[3-(trifluoromethyl)phenyl]-1,3,5-triazine-2,4-diamine

LCMS: M+1=395.3; 1H NMR (DMSO-d6, 90° C., ppm): 1=0.92 t (3H), 3.62 m (2H), 3.21q (2H), 4.98 m (2H), 7.49 m (3H), 7.88 s (1H), 8.42 s (1H), 9.62 s (1H).

### 19. N-butyl-6-(2,2,2-trifluoroethoxy)-N′-[3-(trifluoromethyl)phenyl]-1,3,5-triazine-2,4-diamine

LCMS: M+1=409.3; 1H NMR (DMSO-d6, 90° C., ppm): 1=0.93 t (3H), 1.43 q (2H), 2.60 m (2H), 3.64 m (2H), 4.95 m (2H), 7.36 m (2H), 7.51 t (1H), 7.90 s (1H), 8.21 s (1H), 9.52 s (1H).

### 20. N-pentyl-6-(2,2,2-trifluoroethoxy)-N′-[3-(trifluoromethyl)phenyl]-1,3,5-triazine-2,4-diamine

LCMS: M+1=423.3; 1H NMR (DMSO-d6, 90° C., ppm): 1=0.89 t (3H), 1.35 m (4H), 1.65 m (2H), 3.43 m (2H), 4.92q (2H), 7.39 m (2H), 7.49 t (1H), 7.95 s (1H), 8.16 s (1H), 9.61 s (1H).

### 21. N-hexyl-6-(2,2,2-trifluoroethoxy)-N′-[3-(trifluoromethyl)phenyl]-1,3,5-triazine-2,4-diamine

LCMS: M+1=437.3; 1H NMR (DMSO-d6, 90° C., ppm): 1=0.87 m (8H), 1.31 m (4H), 1.62 m (2H), 3.33 m (2H), 4.98 m (2H), 7.31 m (2H), 7.50 t (1H), 7.85 s (1H), 8.41 s (1H), 9.59 s (1H).

### 22. N-(2-methylpropyl)-6-(2,2,2-trifluoroethoxy)-N′-[3-(trifluoromethyl)phenyl]-1,3,5-triazine-2,4-diamine

LCMS: M+1=409.3; 1H NMR (DMSO-d6, 90° C., ppm): 1=0.95 d (6H), 1.92 m (1H), 1.62 m (2H), 3.21 m (2H), 4.94 m (2H), 7.30 m (2H), 7.51 t (1H), 7.83 s (1H), 8.42 s (1H), 9.60 s 1H).

### 23. 6-(2,2,2-trifluoroethoxy)-N-(2,2,2-trifluoroethyl)-N′-[3-(trifluoromethyl)phenyl]-1,3,5-triazine-2,4-diamine

LCMS: M+1=435.2; 1H NMR (DMSO-d6, 90° C., ppm): 1=4.15 m (2H), 5.01 m (2H), 7.35 m (2H), 7.56 t (1H), 8.13 m (3H), 9.85 s (1H).

### 24. 4-(azetidin-1-yl)-6-(2,2,2-trifluoroethoxy)-N-[3-(trifluoromethyl)phenyl]-1,3,5-triazin-2-amine

LCMS: M+1=393.2; 1H NMR (DMSO-d6, 90° C., ppm): 1=2.32 m (2H), 4.16 m (2H), 4.95 m (2H), 7.36 m (1H), 7.52 t (1H), 7.88 m (1H), 8.34 s (1H), 9.78 s (1H).

### 25. N-cyclobutyl-6-(2,2,2-trifluoroethoxy)-N′-[3-(trifluoromethyl)phenyl]-1,3,5-triazine-2,4-diamine

LCMS: M+1=408.5 NMR 1H, DMSO-d6 δ, ppm: 1.73 m (2H); 2.07 m (2H); 2.30 m (2H); 4.43 m (1H); 4.95 m (2H); 7.32 d (1H); 7.50 t (1H); 7.62 d (1H); 7.90 d (1H); 8.24 s (1H); 9.50 s (1H).

### 26. N-(prop-2-en-1-yl)-6-(2,2,2-trifluoroethoxy)-N′-[3-(trifluoromethyl)phenyl]-1,3,5-triazine-2,4-diamine

LCMS: M+1=393.2; 1H NMR (DMSO-d6, 90° C., ppm): 1=3.99 d (2H), 4.99 m (2H), 5.12 m (2H), 5.92 m (1H), 7.32 m (1H), 7.51 m (2H), 7.92 m (1H), 8.20 s (1H), 9.59 s (1H).

### 27. methyl N-[4-(2,2,2-trifluoroethoxy)-6-{[3-(trifluoromethyl)phenyl]amino}-1,3,5-triazin-2-yl]glycinate

1H NMR (DMSO-d6, 90° C., ppm): d=3.66 s (3H, CH3), 4.09 d (2H, CH2), 4.93 q (2H, CH2), 7.32 d (1H, Ar), 7.50 t (1H, Ar), 7.67 m br. (1H, NH), 7.92 d br. (1H, NH), 8.11 s (1H, Ar), 9.63 s (1H, NH). LC-MS [M+1]: calc'd: 426.3; obs'd: 426.2.

### 28. ethyl N-[4-(2,2,2-trifluoroethoxy)-6-{[3-(trifluoromethyl)phenyl]amino}-1,3,5-triazin-2-yl]glycinate

1H NMR (DMSO-d6, 90° C., ppm): d=1.19 t (3H, CH3), 4.03-4.19 m (4H, 2CH2), 4.93 q (2H, CH2), 7.32 d (1H, Ar), 7.50 t (1H, Ar), 7.64 m br. (1H, NH), 7.91 d br. (1H, NH), 8.11 s (1H, Ar), 9.62 s (1H, NH). LC-MS [M+1]: calc'd: 440.3; obs'd: 440.2.

### 29. propyl N-[4-(2,2,2-trifluoroethoxy)-6-{[3-(trifluoromethyl)phenyl]amino}-1,3,5-triazin-2-yl]glycinate

LC-MS [M+1]: calc'd: 454.3; obs'd: 454.3. Z221-0391emf (NMR-1H) 1H NMR (DMSO-d6, 90° C., ppm): d=0.87 t (3H, CH3), 1.60 m (2H, CH2), 4.00-4.14 m (4H, 2CH2), 4.92 q (2H, CH2), 7.32 d (1H, Ar), 7.49 m (2H, Ar and NH), 7.92 d (1H, Ar), 8.10 s (1H, Ar), 9.48 s (1H, NH).

### 30. N-methyl-N-2-[4-(2,2,2-trifluoroethoxy)-6-{[3-(trifluoromethyl)phenyl]amino}-1,3,5-triazin-2-yl]glycinamide

1H NMR (DMSO-d6, 90° C., ppm): d=2.64 d (3H, CH3), 3.93 d (2H, CH2), 4.94 q (2H, CH2), 7.32 d (1H, Ar), 7.49 m (3H, Ar), 7.98 t (1H, Ar), 8.10 s (1H, Ar), 9.66 s (1H, NH).

LC-MS [M+1]: calc'd: 424.3; obs'd: 425.3.

### 31. N-ethyl-N-2-[4-(2,2,2-trifluoroethoxy)-6-{[3-(trifluoromethyl)phenyl]amino}-1,3,5-triazin-2-yl]glycinamide

1H NMR (DMSO-d6, 90° C., ppm): d=1.06 t (3H, CH3), 3.14 t (2H, CH2), 3.94 d (2H, CH2), 4.94 q (2H, CH2), 7.32 d (1H, Ar), 7.52 t (3H, Ar), 8.00 d (1H, Ar), 8.10 s (1H, Ar), 9.70 s (1H, NH). LC-MS [M+1]: calc'd: 438.3; obs'd: 439.3.

### 32. N-propyl-N-2-[4-(2,2,2-trifluoroethoxy)-6-{[3-(trifluoromethyl)phenyl]amino}-1,3,5-triazin-2-yl]glycinamide

1H NMR (DMSO-d6, 90° C., ppm): d=0.85 t (3H, CH3), 1.45 q (2H, CH2), 3.08 q (2H, CH2), 3.02 t (2H, CH2), 3.94 d (2H, CH2), 4.94 q (2H, CH2), 7.32 d (1H, Ar), 7.42 s (1H, CH), 7.52 t (2H, 2CH), 7.98 d (1H, Ar), 8.10 s (1H, Ar), 9.60 s (1H, NH). LC-MS [M+1]: calc'd: 452.3; obs'd: 453.3.

### 33. N-benzyl-N-2-[4-(2,2,2-trifluoroethoxy)-6-{[3-(trifluoromethyl)phenyl]amino}-1,3,5-triazin-2-yl]glycinamide

1H NMR (DMSO-d6, 90° C., ppm): d=4.02 d (2H, CH2), 4.32 d (2H, CH2), 4.92 q (2H, CH2), 7.26 m (5H, Ar), 7.50 t (2H, CH), 8.01 t (2H, Ar), 8.11 s (1H, Ar), 9.64 s (1H, NH). LC-MS [M+1]: calc'd: 500.4; obs'd: 501.5.

### 34. N-phenyl-N-2-[4-(2,2,2-trifluoroethoxy)-6-{[3-(trifluoromethyl)phenyl]amino}-1,3,5-triazin-2-yl]glycinamide

1H NMR (DMSO-d6, 90° C., ppm): d=4.15 d (2H, CH2), 4.94 q (2H, CH2), 7.05 t (1H, Ar), 7.32 d (3H, Ar), 7.48 s (1H, CH), 7.58 d (3H, Ar), 8.02 d (1H, Ar), 8.09 s (1H, Ar), 9.68 s (1H, NH).

LC-MS [M+1]: calc'd: 486.3; obs'd: 487.3.

### 35. N-[4-(2,2,2-trifluoroethoxy)-6-{[3-(trifluoromethyl)phenyl]amino}-1,3,5-triazin-2-yl]acetamide

LCMS: M+1=396.1; NMR 1H, DMSO-d6 δ, ppm: 2.32 s (3H); 5.00 m (2H); 7.35 d (1H); 7.51 t (1H); 8.08 d (1H); 8.16 s (1H); 10.00 s (2H).

### 36. N-[4-(2,2,2-trifluoroethoxy)-6-{[3-(trifluoromethyl)phenyl]amino}-1,3,5-triazin-2-yl]propanamide

LCMS: M+1=410.1 NMR 1H, DMSO-d6 δ, ppm: 1.10 t (3H); 2.65 q (2H); 5.00 m (2H); 7.35 d (1H); 7.54 t (1H); 8.08 d (1H); 8.16 s (1H); 9.90 s (2H).

### 37. N-[4-(2,2,2-trifluoroethoxy)-6-{[3-(trifluoromethyl)phenyl]amino}-1,3,5-triazin-2-yl]butanamide

LCMS: M+1=424.3; NMR 1H, DMSO-d6 δ, ppm: 0.92 t (3H); 1.58 q (2H); 5.05 m (2H); 7.35 d (1H); 7.54 t (1H); 8.08 d (1H); 8.16 s (1H); 10.60 s (2H).

### 38. 2-methyl-N-[4-(2,2,2-trifluoroethoxy)-6-{[3-(trifluoromethyl)phenyl]amino}-1,3,5-triazin-2-yl]propanamide

LCMS: M+1=424.3; NMR 1H, DMSO-d6 δ, ppm: 1.14 d (6H); 3.03 m (1H); 5.00 m (2H); 7.35 d (1H); 7.54 t (1H); 8.08 d (1H); 8.18 s (1H); 9.90 s (2H).

### 39. methyl N-[4-(2,2,2-trifluoroethoxy)-6-{[3-(trifluoromethyl)phenyl]amino}-1,3,5-triazin-2-yl]-beta-alaninate

1H NMR (DMSO-d6, 90° C., ppm): d=2.62 t (2H, CH2), 3.60 m (2H, CH2), 3.62 s (3H, CH3), 4.93 q (2H, CH2), 7.31 d (1H, Ar), 7.38 s br. (1H, NH), 7.50 t (1H, Ar), 7.93 d (1H, NH), 8.17 s (1H, Ar), 9.56 s (1H, NH). LC-MS [M+1]: calc'd: 440.3; obs'd: 440.2.

### 40. ethyl N-[4-(2,2,2-trifluoroethoxy)-6-{[3-(trifluoromethyl)phenyl]amino}-1,3,5-triazin-2-yl]-beta-alaninate

1H NMR (DMSO-d6, 90° C., ppm): d=2.28 q (2H, CH2), 2.62 t (2H, CH2), 3.60 m (2H, CH2), 3.32 t (3H, CH3), 4.93 q (2H, CH2), 7.31 d (1H, Ar), 7.38 s br. (1H, NH), 7.50 t (1H, Ar), 7.93 d (1H, NH), 8.17 s (1H, Ar), 9.56 s (1H, NH). LC-MS [M+1]: calc'd: 440.3; obs'd: 440.2.

### 41. propyl N-[4-(2,2,2-trifluoroethoxy)-6-{[3-(trifluoromethyl)phenyl]amino}-1,3,5-triazin-2-yl]-beta-alaninate

LC-MS [M+1]: calc'd: 468.4; obs'd: 468.3. 1H NMR (DMSO-d6, 90° C., ppm): d=0.89 t (3H, CH3), 1.60 m (2H, CH2), 2.62 t (2H, CH2), 3.62 q (2H, CH2), 4.01 t (2H, CH2), 4.92 q (2H, CH2), 7.22 s br. (1H, NH), 7.31 d (1H, Ar), 7.49 t (1H, Ar), 7.93 d (1H, Ar), 8.14 (1H, Ar), 9.43 s (1H, NH).

### 42. N-methyl-N-3-[4-(2,2,2-trifluoroethoxy)-6-{[3-(trifluoromethyl)phenyl]amino}-1,3,5-triazin-2-yl]-beta-alaninamide

1H NMR (DMSO-d6, 90° C., ppm): d=2.42 t (2H, CH2), 2.62 s (3H, CH3), 3.58 t (2H, CH2), 4.93 q (2H, CH2), 7.30 d (1H, Ar), 7.50 t (1H, Ar), 7.97 d (1H, Ar), 8.17 s (1H, Ar).

LC-MS [M+1]: calc'd: 438.3; obs'd: 439.5.

### 43. N-ethyl-N-3-[4-(2,2,2-trifluoroethoxy)-6-{[3-(trifluoromethyl)phenyl]amino}-1,3,5-triazin-2-yl]-beta-alaninamide

1H NMR (DMSO-d6, 90° C., ppm): d=1.04 t (3H, CH2), 2.41 t (2H, CH2), 3.12 m (2H, CH2), 3.58 q (2H,), 4.94 q (2H, CH2), 7.23 s (1H, Ar), 7.32 d (1H, Ar), 7.42 s (1H, Ar), 7.51 t (1H, Ar), 7.97 d (1H, NH), 8.17 s (1H, NH), 9.55 s (1H, NH). LC-MS [M+1]: calc'd: 452.3; obs'd: 453.5.

### 44. N-propyl-N-3-[4-(2,2,2-trifluoroethoxy)-6-{[3-(trifluoromethyl)phenyl]amino}-1,3,5-triazin-2-yl]-beta-alaninamide

1H NMR (DMSO-d6, 90° C., ppm): d=0.87 t (3H, CH3), 1.46 m (2H, CH2), 2.43 t (2H, CH2), 3.07 q (2H, CH2), 3.60 q (2H, CH2), 4.92 q (2H, CH2), 7.09 s (1H, Ar), 7.31 d (2H, Ar), 7.50 t (1H, Ar), 7.96 d (1H, NH), 8.15 s (1H, NH), 9.43 s (1H, NH). LC-MS [M+1]: calc'd: 466.4; obs'd: 467.3.

### 45. N-benzyl-N-3-[4-(2,2,2-trifluoroethoxy)-6-{[3-(trifluoromethyl)phenyl]amino}-1,3,5-triazin-2-yl]-beta-alaninamide

1H NMR (DMSO-d6, 90° C., ppm): d=3.63 q (2H, CH2), 4.31 d (2H, CH2), 4.92 q (2H, CH2), 7.08 s (1H, Ar), 7.22 t (3H, Ar), 7.31 d (2H, Ar), 7.49 t (1H, Ar), 7.80 s (1H, Ar), 7.96 d (1H, Ar), 8.13 s (1H, NH), 9.38 s (1H, NH). LC-MS: Z221-0417.jpg; LC-MS [M+1]: calc'd: 514.4; obs'd: 515.2.

### 46. N-phenyl-N-3-[4-(2,2,2-trifluoroethoxy)-6-{[3-(trifluoromethyl)phenyl]amino}-1,3,5-triazin-2-yl]-beta-alaninamide

1H NMR (DMSO-d6, 90° C., ppm): d=2.65 t (2H, CH2), 3.68 q (2H, CH2), 4.94 q (2H, CH2), 7.03 t (1H, Ar), 7.29 q (3H, Ar), 7.41 m (1H, Ar), 7.54 m (3H, Ar), 8.18 s (1H, NH), 9.58 s (2H, NH). LC-MS [M+1]: calc'd: 500.4; obs'd: 501.5.

### 47. methyl 4-{[4-(2,2,2-trifluoroethoxy)-6-{[3-(trifluoromethyl)phenyl]amino}-1,3,5-triazin-2-yl]amino}butanoate

LC-MS [M+1]: calc'd: 454.3; obs'd: 454.3. 1H NMR (DMSO-d6, 90° C., ppm): d=1.87 m (2H, CH2), 2.37 t (2H, CH2), 3.39 q (2H, CH2), 3.60 s (3H, CH3), 4.92 q (2H, CH2), 7.18-7.38 m br. (2H, Ar and NH), 7.49 t (1H, Ar), 7.93 d (1H, Ar), 8.16 s (1H, Ar), 9.39 s (1H, NH).

### 48. ethyl 4-{[4-(2,2,2-trifluoroethoxy)-6-{[3-(trifluoromethyl)phenyl]amino}-1,3,5-triazin-2-yl]amino}butanoate

1H NMR (DMSO-d6, 90° C., ppm): d=1.18 t (3H, CH3), 1.85 m (2H, CH2), 2.34 t (2H, CH2), 3.38 q (2H, CH2), 4.06 q (2H, CH2), 4.93 q (2H, CH2), 7.31 d (1H, Ar), 7.41 s br. (1H, NH), 7.50 t (1H, Ar), 7.93 d (1H, NH), 8.18 s (1H, Ar), 9.53 s (1H, NH). LC-MS [M+1]: calc'd: 468.4; obs'd: 468.2.

### 49. propyl 4-{[4-(2,2,2-trifluoroethoxy)-6-{[3-(trifluoromethyl)phenyl]amino}-1,3,5-triazin-2-yl]amino}butanoate

LC-MS [M+1]: calc'd: 482.4; obs'd: 482.3. 1H NMR (DMSO-d6, 90° C., ppm): d=0.89 t (3H, CH3), 1.59 m (2H, CH2), 1.87 m (2H, CH2), 2.36 t (2H, CH2), 3.40 d (2H, CH2), 3.99 t (2H, CH2), 4.92 q (2H, CH2), 7.16-7.39 m br. (2H, Ar and NH), 7.49 t (1H, Ar), 7.93 d (1H, Ar), 8.17 s (1H, Ar), 9.44 s br. (1H, NH).

### 50. N-ethyl-4-{[4-(2,2,2-trifluoroethoxy)-6-{[3-(trifluoromethyl)phenyl]amino}-1,3,5-triazin-2-yl]amino}butanamide

1H NMR (DMSO-d6, 90° C., ppm): d=1.03 t (3H, CH3), 1.82 q (2H, CH2), 2.15 t (2H, CH2), 3.10 m (2H, CH2), 3.37 q (2H, CH2), 4.94 q (2H, CH2), 7.31 d (1H, Ar), 7.40 m (2H, Ar), 7.51 t (1H, Ar), 7.95 d (1H, Ar), 8.20 s (1H, Ar), 9.55 s (1H, NH). LC-MS [M+1]: calc'd: 466.3; obs'd: 467.3.

### 51. N-propyl-4-{[4-(2,2,2-trifluoroethoxy)-6-{[3-(trifluoromethyl)phenyl]amino}-1,3,5-triazin-2-yl]amino}butanamide

1H NMR (DMSO-d6, 90° C., ppm): d=2.62 t (2H, CH2), 3.60 m (2H, CH2), 3.62 s (3H, CH3), 4.93 q (2H, CH2), 7.31 d (1H, Ar), 7.38 s br. (1H, NH), 7.50 t (1H, Ar), 7.93 d (1H, NH), 8.17 s (1H, Ar), 9.56 s (1H, NH). LC-MS [M+1]: calc'd: 440.3; obs'd: 440.2.

### 52. N-phenyl-4-{[4-(2,2,2-trifluoroethoxy)-6-{[3-(trifluoromethyl)phenyl]amino}-1,3,5-triazin-2-yl]amino}butanamide

1H NMR (DMSO-d6, 90° C., ppm): d=2.62 t (2H, CH2), 3.60 m (2H, CH2), 3.62 s (3H, CH3), 4.93 q (2H, CH2), 7.31 d (1H, Ar), 7.38 s br. (1H, NH), 7.50 t (1H, Ar), 7.93 d (1H, NH), 8.17 s (1H, Ar), 9.56 s (1H, NH). LC-MS [M+1]: calc'd: 440.3; obs'd: 440.2.

### 53. N-[2-(6-methoxy-1H-indol-3-yl)ethyl]-6-(2,2,2-trifluoroethoxy)-N′-[3-(trifluoromethyl)phenyl]-1,3,5-triazine-2,4-diamine

LCMS: M+1=527.2 NMR 1H, DMSO-d6 δ, ppm: 3.00 m (2H); 3.60 m (2H); 4.00 s (3H); 4.90 m (2H); 6.64 d (1H); 6.88 s (1H); 6.98 s (1H); 7.26-7.52 m (4H); 7.96 d (1H); 8.16 s (1H); 10.3 s (1H).

### 54. N-[3-(1H-imidazol-1-yl)propyl]-6-(2,2,2-trifluoroethoxy)-N′-[3-(trifluoromethyl)phenyl]-1,3,5-triazine-2,4-diamine

LCMS: M+1=462.2 NMR 1H, DMSO-d6 δ, ppm: 2.00 m (2H); 3.35 m (2H); 4.05 m (2H); 4.95 m (2H); 6.88 s (1H); 7.10 s (1H); 7.32 d (1H); 7.44-7.60 m (3H); 7.94 d (1H); 8.16 s (1H); 9.55 s (1H).

### 55. N-(4-phenylbutyl)-6-(2,2,2-trifluoroethoxy)-N′-[3-(trifluoromethyl)phenyl]-1,3,5-triazine-2,4-diamine

LCMS: M+1=486.5 NMR 1H, DMSO-d6 δ, ppm: 1.65 m (4H); 2.65 m (2H); 3.87 m (2H); 4.92 m (2H); 7.10-7.34 m (7H); 7.37-7.55 s (2H); 9.52 s (1H).

### 56. 1-[4-(2,2,2-trifluoroethoxy)-6-{[3-(trifluoromethyl)phenyl]amino}-1,3,5-triazin-2-yl]pyrrolidin-3-one

LCMS: M+1=422; NMR 1H, DMSO, ppm: 2.64 m (2H, CH); 4.02 m (4H CH); 4.88 m (2H, CH); 7.22 d (1H, CH); 7.45 t (1H, CH); 7.88 d (1H, CH), 8.28 s (1H, CH), 10.1 s (1H, NH)

### 57. pyridin-3-yl[4-{[4-(2,2,2-trifluoroethoxy)-6-{[3-(trifluoromethyl)phenyl]amino}-1,3,5-triazin-2-yl]amino}methyl)piperidin-1-yl]methanone

1H NMR (DMSO, ppm) 1.12 m (2H), 1.82 d (2H), 1.98 bs (1H), 3.0 m (2H), 3.28 m (2H), 4.0 bs (2H), 4.48 m (2H), 7.28 d (1H), 7.48 m (3H), 7.78 d (1H), 7.88 bs (1H), 8.24 bs (1H), 8.6 m (2H), 9.48 s (1H); LCMS: M+1=556

### 58. 3-(3,5-dimethyl isoxazol-4-yl)-1-[4-({[4-(2,2,2-trifluoroethoxy)-6-{[3-(trifluoromethyl)phenyl]amino}-1,3,5-triazin-2-yl]amino}methyl)piperidin-1-yl]propan-1-one

1H NMR (DMSO, ppm) 1.12 m (2H), 1.52 s (1H), 1.8 m (4H), 2.18 s (3H), 2.28 s (3H), 2.5 m (2H), 2.8 bs (2H), 3.25 m (2H), 4.02 bs (2H), 4.98 m (2H), 7.26 d (1H), 7.56 m (2H), 7.88 bs (1H), 8.2 bs (1H), 9.3 bs (1H); LCMS: M+1=602

### 59. N,N-dimethyl-4-{[4-(2,2,2-trifluoroethoxy)-6-{[3-(trifluoromethyl)phenyl]amino}-1,3,5-triazin-2-yl]amino}methyl)piperidine-1-sulfonamide

1H NMR (DMSO, ppm) 1.14 m (2H), 1.18 m (3H), 2.82 m (8H), 3.34 m (2H), 3.52 m (2H), 4.98 m (2H), 7.28 d (1H), 7.48 m (2H), 7.86 bs (1H), 8.2 bs (1H), 9.62 s (1H); LCMS: M+1=558

### 60. N-[4-(methylsulfonyl)phenyl]-6-(2,2,2-trifluoroethoxy)-N′-[3-(trifluoromethyl)phenyl]-1,3,5-triazine-2,4-diamine

LCMS: M+1=508.4 NMR 1H, DMSO-d6 δ, ppm: 3.01 s (3H); 5.00 m (2H); 7.42 d (1H); 7.58 t (1H); 7.80-8.10 m (6H); 10.00 (1H); 10.04 s (1H).

### 61. N-phenyl-6-(2,2,2-trifluoroethoxy)-N′-[3-(trifluoromethyl)phenyl]-1,3,5-triazine-2,4-diamine

LCMS: M+1=430.5; 1H NMR, DMSO-d6 δ, ppm: 4.60 m (2H); 5.00 m (2H); 6.78 d (1H); 7.20-7.46 m (4H); 7.53 t (1H); 8.00 d (2H); 9.55 s (1H); 9.75 s (1H).

### 62. N-(4-fluorophenyl)-4-[(4-(2,2,2-trifluoroethoxy)-6-{[3-(trifluoromethyl)phenyl]amino}-1,3,5-triazin-2-yl)amino]benzenesulfonamide

LCMS: M+1=603.5 NMR 1H, DMSO-d6 δ, ppm: 5.00 m (2H); 5.94-7.18 m (4H); 7.34-7.70 m (4H); 7.78-8.08 m (4H); 9.70-10.00 (3H).

### 63. N-(2-methylphenyl)-4-[(4-(2,2,2-trifluoroethoxy)-6-{[3-(trifluoromethyl)phenyl]amino}-1,3,5-triazin-2-yl)amino]benzenesulfonamide

LCMS: M+1=599.3 NMR 1H, DMSO-d6 δ, ppm: 2.05 s (3H); 5.00 m (2H); 6.98-7.18 m (4H); 3.38 d (1H); 7.48-7.66 m (3H); 7.86 d (2H); 7.90-8.08 d (2H); 9.05 s (1H); 9.95 (1H); 10.00 s (1H).

### 64. 6-(2,2,2-trifluoroethoxy)-N-[3-(2,2,2-trifluoroethoxy)phenyl]-N′-[3-(trifluoromethyl)phenyl]-1,3,5-triazine-2,4-diamine

LCMS: M+1=528.4 NMR 1H, DMSO-d6 δ, ppm: 5.00 m (2H); 7.18 t (1H); 7.26-7.40 m (3H); 7.52 t (1H); 7.66 d (2H); 7.98 d (1H); 8.08 s (1H); 9.50 s (1H); 9.75 s (1H).

### Generic Synthesis of Intermediates and Final Compounds for Table 36

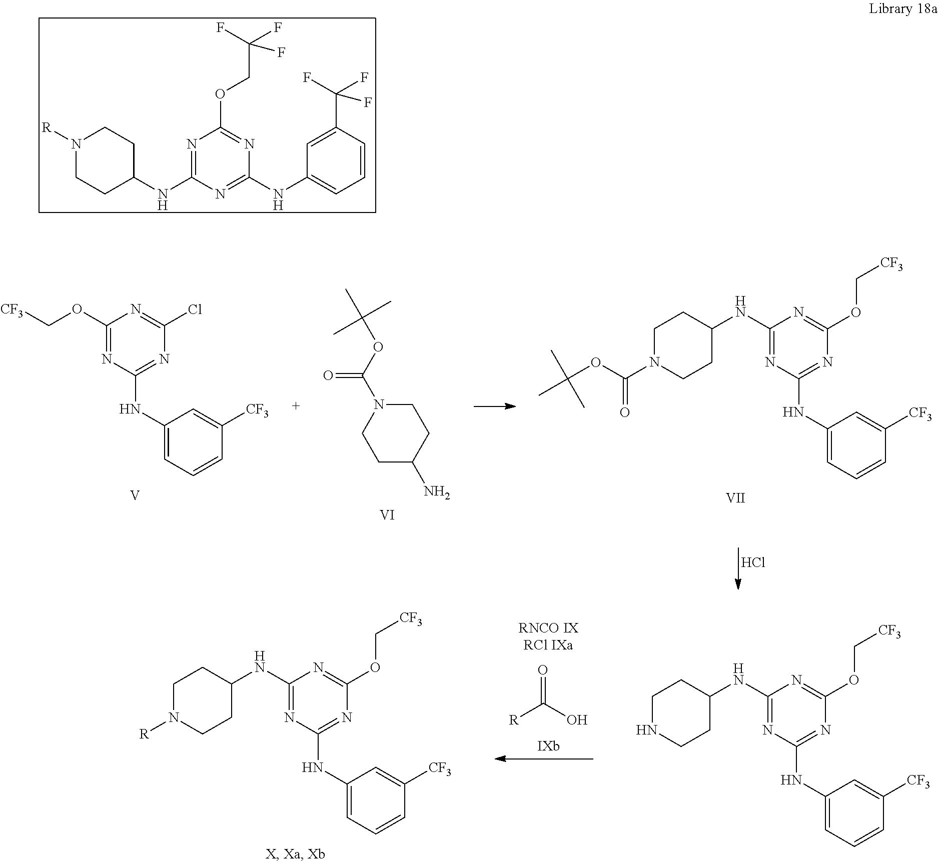

Preparation of 6-trifluoroethoxy-N-[3-(trifluoromethyl)phenyl]-1,3,5-triazine-2-(tert-butyl-4-(aminopyperidinecarboxylate VII. To a stirred solution of intermediate V (5 g) and triethylamine (1.63 g) in 200 ml of dioxane was added portionwise compound VI (2.68 g). After stirring for 7 h at rt, the reaction mixture was checked for completion by LCMS and the solvent was removed under reduced pressure. The residue was treated with water, extracted with CH₂Cl₂, and the organic phase was dried over sodium sulfate. The organic layer was then treated with acetonitrile and filtered. The precipitate formed was washed with ether to provide Boc-amine VII as white solid, Yield 41% (3 g). LCMS: M+1=537.5.

Preparation of 6-trifluoroethoxy-N-[3-(trifluoromethyl)phenyl]-1,3,5-triazine-2-4-amino-piperidine VIII. Compound VII (1 g) was dissolved in saturated dioxane-HCl solution (10 ml) and stirred at rt for 5 h. The solvent was removed under reduced pressure and the resultant residue was treated with water, neutralized with 10% aqueous KOH solution, filtered and washed with water to provide VIII as white solid. Yield 75% (0.61 g). LCMS: M+1=437.3.

Compound X (Urea) was obtained according to the following procedure: A solution of 10 mmol of intermediate VIII in 10 ml of dioxane was treated with isocyanate IX (10 mmol). This mixture was stirred at reflux for 3 hours, then cooled and poured into water. The precipitate formed was filtered, washed with water, and dried. The precipitate was purified by column chromatography.

Compound Xa (Amide). Carboxylic acid IXb (10 mmol) was dissolved in 2 ml of DMF and CDI (0.11 mmol) was added. The reaction mixture was stirred for 1 hour at ambient temperature, then treated with a solution of intermediate VIII (10 mmol) in 2 ml of DMF. This mixture was stirred at 70° C. for 2 hours, then cooled and concentrated under reduced pressure. The residue was washed with 10% aqueous NaHCO₃, then with water, dried, and purified by column chromatography.

Compound Xb. A solution of intermediate VIII (10 mmol) in 10 ml of isopropanol was treated with alkyl halide IXa (10 mmol) and DIPEA (22 mmol). This mixture was stirred at reflux for 3 hours, then cooled and concentrated. The residue was treated with water. The resulting precipitate was filtered, washed with water, dried, and purified by column chromatography.

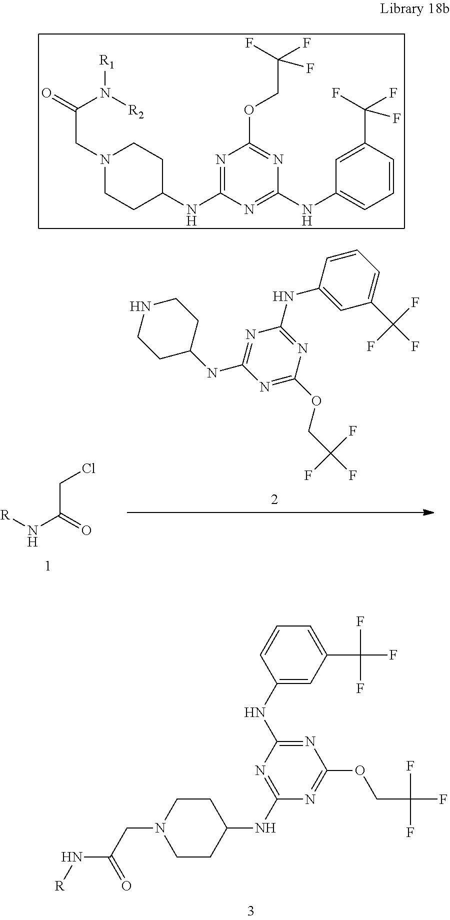

Compound 3 was synthesized according to the following procedure: A solution of intermediate 2 (1 mmol) in 5 ml of isopropanol was treated with alkyl chloride 1 (1 mmol) and NEt₃ (4 mmol). This mixture was stirred at reflux for 5 hours, then cooled and concentrated. Residue was treated with water. The formed precipitate was filtered off, washed with water, dried and purified by column chromatography.

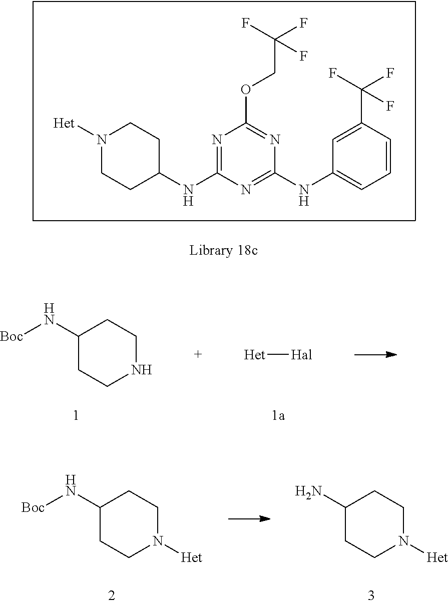

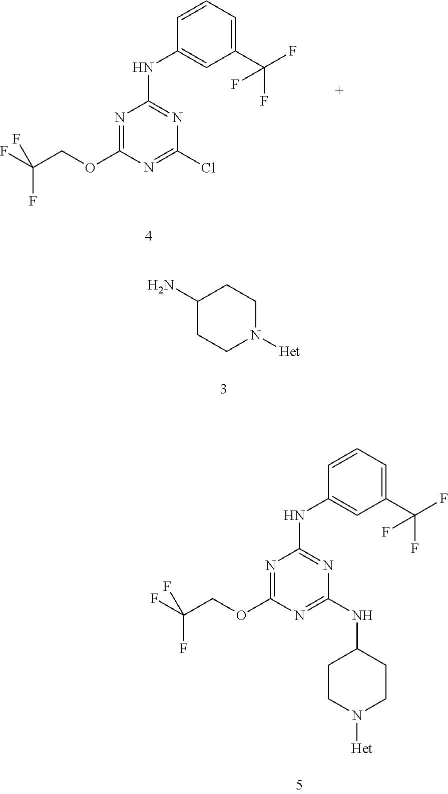

Preparation of Intermediate 2.1 g (0.005 mol) of compound 1 was dissolved in 50 ml of DMF, then 0.005 mol of halo-heterocycles 1a and 0.01 mol of potassium carbonate were added. Reaction mixture stirred at 80° C. for about 48 hours. LCMS of reaction mixture demonstrated about 85% of compound 2. The precipitate was filtered off, washed with ether and lyophilized.

Preparation of Intermediate 3.1 g (0.0030 mol) of compound 2 was suspended in 50 ml of dioxane with HCl. The reaction mixture was allowed to stir overnight at rt. LCMS analysis of the reaction mixture demonstrated total conversion of starting material; precipitate was filtered, washed with ether and lyophilized. Compound 3 was obtained as hydrochloride.

Preparation of Compound 5. 0.5 g (0.0016 mol) of compound 3 was suspended in 5 ml of dry dioxane, then 1 ml of TEA was added. The reaction mixture was stirred for about 3-5 min and 0.5 g (0.0013 mol) of compound 4 were added. The reaction mixture was stirred at 80° C. for about 48 hours. LCMS analysis of the reaction mixture demonstrated about 60% of compound 5. The reaction mixture was poured into water, extracted with chloroform, and allowed to sit overnight to allow the product to slowly crystallize from chloroform. The crystals of final product were then collected.

TABLE 36

| En | Structure | IUPAC Name | MW | Formula |
|---|---|---|---|---|
| 1 | 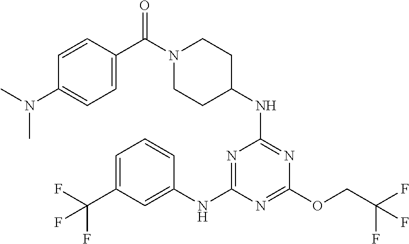 | [4-(dimethylamino)phenyl](4-{[4-(2,2,2- trifluoro-ethoxy)-6-{[3- (trifluoromethyl)phenyl]amino}-1,3,5- triazin-2-yl]amino}piperidin-1- yl)methanone | 583.5 | C₂₆H₂₇F₆N₇O₂ |
| 2 | 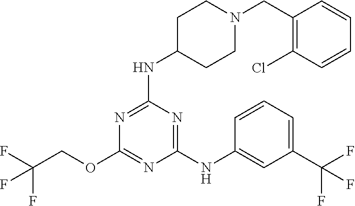 | N-[1-(2-chlorobenzyl)piperidin-4-yl]-6- (2,2,2-trifluoroethoxy)-N′-[3- (trifluoromethyl)phenyl]-1,3,5-triazine-2,4- diamine | 560.9 | C₂₄H₂₃ClF₆N₆O |
| 3 | 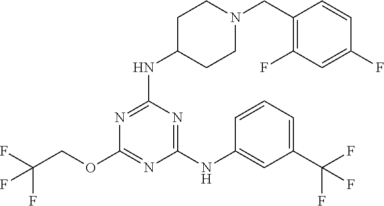 | N-[1-(2,4-difluorobenzyl)piperidin-4-yl]-6- (2,2,2-trifluoroethoxy)-N′-[3- (trifluoromethyl)phenyl]-1,3,5-triazine-2,4- diamine | 562.5 | C₂₄H₂₂F₈N₆O |
| 4 | 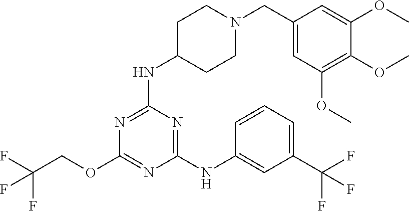 | 6-(2,2,2-trifluoroethoxy)-N-[3- (trifluoromethyl)-phenyl]-N′-[1-(3,4,5- trimethoxybenzyl)piperidin-4-yl]-1,3,5- triazine-2,4-diamine | 616.6 | C₂₇H₃₀F₆N₆O₄ |
| 5 | 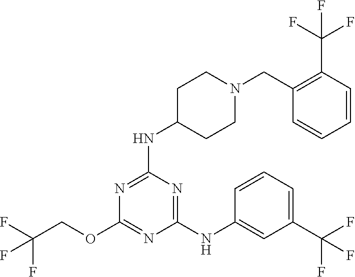 | 6-(2,2,2-trifluoroethoxy)-N-{1-[2- (trifluoromethyl)benzyl]piperidin-4-yl}-N′- [3-(trifluoromethyl)phenyl]-1,3,5-triazine- 2,4-diamine | 594.5 | C₂₅H₂₃F₉N₆O |
| 6 | 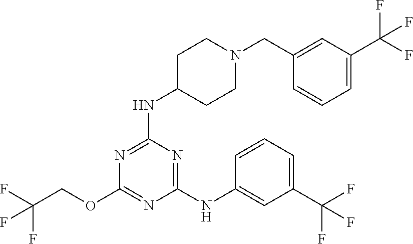 | 6-(2,2,2-trifluoroethoxy)-N-{1-[3- (trifluoromethyl)benzyl]piperidin-4-yl}-N′- [3-(trifluoromethyl)phenyl]-1,3,5-triazine- 2,4-diamine | 594.5 | C₂₅H₂₃F₉N₆O |
| 7 | 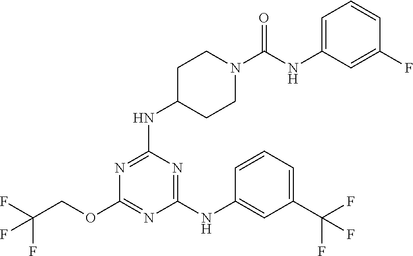 | N-(3-fluorophenyl)-4-{[4-(2,2,2- trifluoroethoxy)-6-{[3- (trifluoromethyl)phenyl]amino}-1,3,5- triazin-2-yl]amino}piperidine-1- carboxamide | 573.5 | C₂₄H₂₂F₇N₇O₂ |
| 8 | 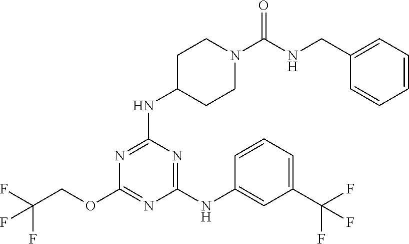 | N-benzyl-4-{[4-(2,2,2-trifluoroethoxy)-6- {[3-(trifluoromethyl)phenyl]amino}-1,3,5- triazin-2-yl]amino}piperidine-1- carboxamide | 569.5 | C₂₅H₂₅F₆N₇O₂ |
| 9 | 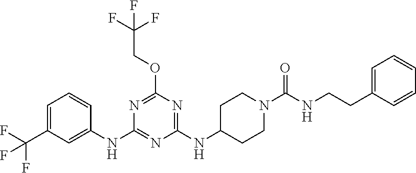 | N-(2-phenylethyl)-4-{[4-(2,2,2- trifluoroethoxy)-6-{[3- (trifluoromethyl)phenyl]amino}-1,3,5- triazin-2-yl]amino}piperidine-1- carboxamide | 583.5 | C₂₆H₂₇F₆N₇O₂ |
| 10 | 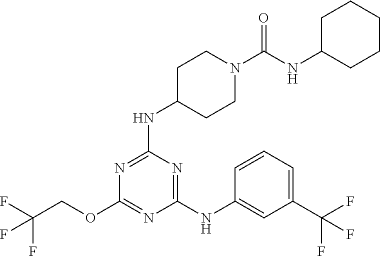 | N-cyclohexyl-4-{[4-(2,2,2-trifluoroethoxy)- 6-{[3-(trifluoromethyl)phenyl]amino}-1,3,5- triazin-2-yl]amino}piperidine-1- carboxamide | 561.5 | C₂₄H₂₉F₆N₇O₂ |
| 11 | 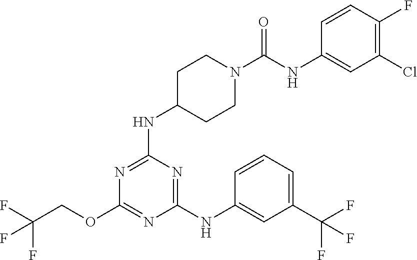 | N-(3-chloro-4-fluorophenyl)-4-{[4-(2,2,2- trifluoro-ethoxy)-6-{[3- (trifluoromethyl)phenyl]amino}-1,3,5- triazin-2-yl]amino}piperidine-1- carboxamide | 607.9 | C₂₄H₂₁ClF₇N₇O₂ |
| 12 | 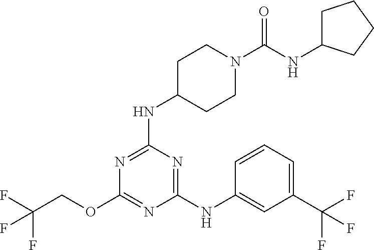 | N-cyclopentyl-4-{[4-(2,2,2-trifluoroethoxy)- 6-{[3-(trifluoromethyl)phenyl]amino}-1,3,5- triazin-2-yl]amino}piperidine-1- carboxamide | 547.5 | C₂₃H₂₇F₆N₇O₂ |
| 13 | 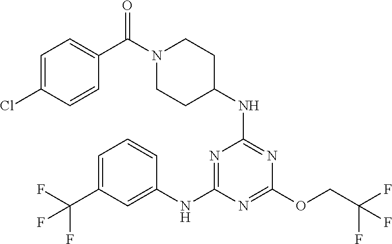 | (4-chlorophenyl)(4-{[4-(2,2,2- trifluoroethoxy)-6-{[3- (trifluoromethyl)phenyl]amino}-1,3,5- triazin-2-yl]amino}piperidin-1- yl)methanone | 574.9 | C₂₄H₂₁ClF₆N₆O₂ |
| 14 | 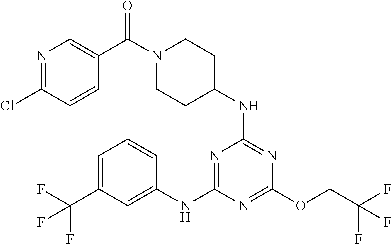 | (6-chloropyridin-3-yl)(4-{[4-(2,2,2- trifluoroethoxy)-6-{[3- (trifluoromethyl)phenyl]amino}-1,3,5- triazin-2-yl]amino}piperidin-1- yl)methanone | 575.9 | C₂₃H₂₀ClF₆N₇O₂ |
| 15 | 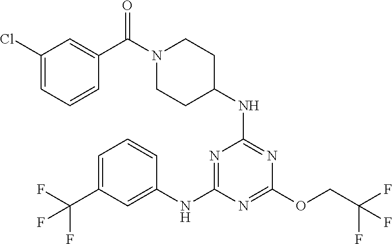 | (3-chlorophenyl)(4-{[4-(2,2,2- trifluoroethoxy)-6-{[3- (trifluoromethyl)phenyl]amino}-1,3,5- triazin-2-yl]amino}piperidin-1- yl)methanone | 574.9 | C₂₄H₂₁ClF₆N₆O₂ |
| 16 | 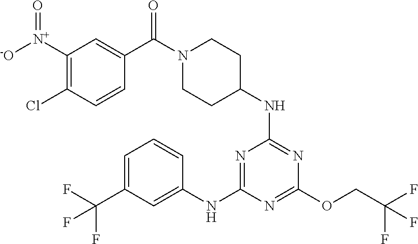 | (4-chloro-3-nitrophenyl)(4-{[4-(2,2,2- trifluoro-ethoxy)-6-{[3- (trifluoromethyl)phenyl]amino}-1,3,5- triazin-2-yl]amino}piperidin-1- yl)methanone | 619.9 | C₂₄H₂₀ClF₆N₇O₄ |
| 17 | 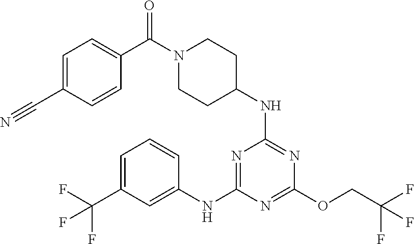 | 4-[(4-{[4-(2,2,2-trifluoroethoxy)-6-{[3- (trifluoromethyl)phenyl]amino}-1,3,5- triazin-2-yl]amino}piperidin-1- yl)carbonyl]benzonitrile | 565.5 | C₂₅H₂₁F₆N₇O₂ |
| 18 | 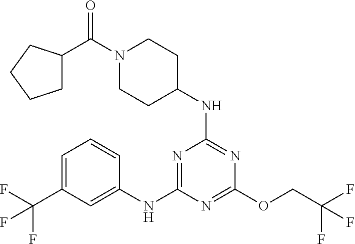 | cyclopentyl(4-{[4-(2,2,2-trifluoroethoxy)-6- {[3-(trifluoromethyl)phenyl]amino}-1,3,5- triazin-2-yl]amino}piperidin-1- yl)methanone | 532.5 | C₂₃H₂₆F₆N₆O₂ |
| 19 | 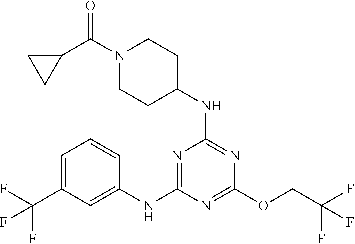 | cyclopropyl(4-{[4-(2,2,2-trifluoroethoxy)-6- {[3-(trifluoromethyl)phenyl]amino}-1,3,5- triazin-2-yl]amino}piperidin-1- yl)methanone | 504.4 | C₂₁H₂₂F₆N₆O₂ |
| 20 | 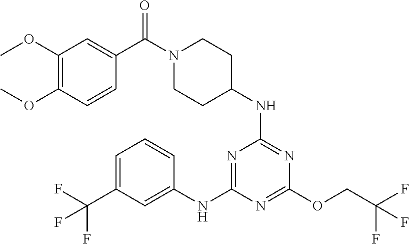 | (3,4-dimethoxyphenyl)(4-{[4-(2,2,2- trifluoro-ethoxy)-6-{[3- (trifluoromethyl)phenyl]amino}-1,3,5- triazin-2-yl]amino}piperidin-1- yl)methanone | 600.5 | C₂₆H₂₆F₆N₆O₄ |
| 21 | 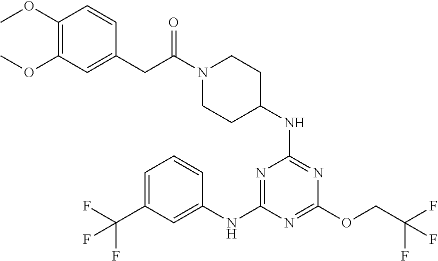 | 2-(3,4-dimethoxyphenyl)-1-(4-{[4-(2,2,2- trifluoro-ethoxy)-6-{[3- (trifluoromethyl)phenyl]amino}-1,3,5- triazin-2-yl]amino}piperidin-1-yl)ethanone | 614.5 | C₂₇H₂₈F₆N₆O₄ |
| 22 | 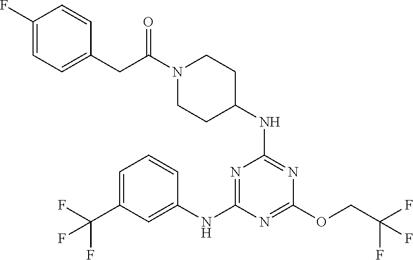 | 2-(4-fluorophenyl)-1-(4-{[4-(2,2,2- trifluoroethoxy)-6-{[3- (trifluoromethyl)phenyl]amino}-1,3,5- triazin-2-yl]amino}piperidin-1-yl)ethanone | 572.5 | C₂₅H₂₃F₇N₆O₂ |
| 23 | 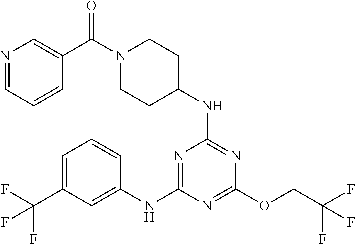 | pyridin-3-yl(4-{[4-(2,2,2-trifluoroethoxy)-6- {[3-(trifluoromethyl)phenyl]amino}-1,3,5- triazin-2-yl]amino}piperidin-1- yl)methanone | 541.5 | C₂₃H₂₁F₆N₇O₂ |
| 24 | 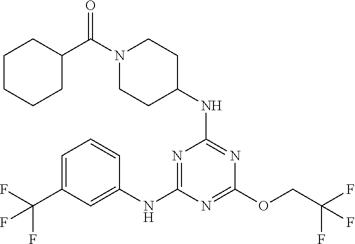 | cyclohexyl(4-{[4-(2,2,2-trifluoroethoxy)-6- {[3-(trifluoromethyl)phenyl]amino}-1,3,5- triazin-2-yl]amino}piperidin-1- yl)methanone | 546.5 | C₂₄H₂₈F₆N₆O₂ |
| 25 | 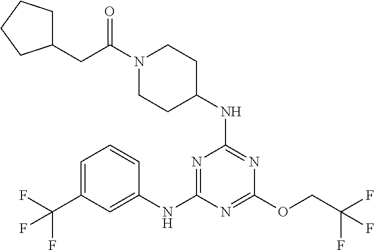 | 2-cyclopentyl-1-(4-{[4-(2,2,2- trifluoroethoxy)-6-{[3- (trifluoromethyl)phenyl]amino}-1,3,5- triazin-2-yl]amino}piperidin-1-yl)ethanone | 546.5 | C₂₄H₂₈F₆N₆O₂ |
| 26 | 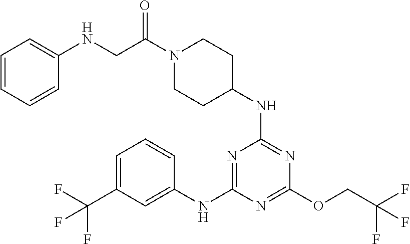 | 2-(phenylamino)-1-(4-{[4-(2,2,2- trifluoroethoxy)-6-{[3- trifluoromethyl)phenyl]amino}-1,3,5- triazin-2-yl]amino}piperidin-1-yl)ethanone | 569.5 | C₂₅H₂₅F₆N₇O₂ |
| 27 | 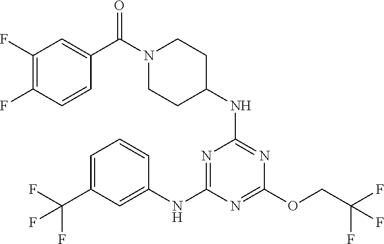 | (3,4-difluorophenyl)(4-{[4-(2,2,2- trifluoroethoxy)-6-{[3- (trifluoromethyl)phenyl]amino}-1,3,5- triazin-2-yl]amino}piperidin-1- yl)methanone | 576.4 | C₂₄H₂₀F₈N₆O₂ |
| 28 | 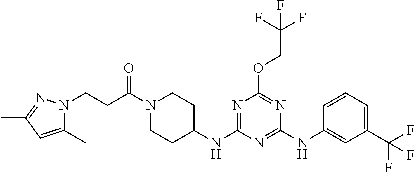 | 3-(3,5-dimethyl-1H-pyrazol-1-yl)-1-(4-{[4- (2,2,2-trifluoroethoxy)-6-{[3- (trifluoromethyl)phenyl]amino}-1,3,5- triazin-2-yl]amino}piperidin-1-yl)propan-1- one | 586.5 | C₂₅H₂₈F₆N₈O₂ |
| 29 | 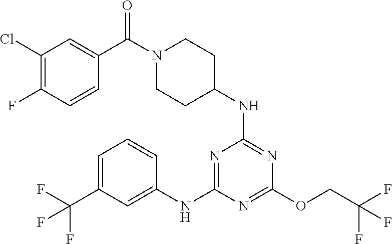 | (3-chloro-4-fluorophenyl)(4-{[4-(2,2,2- trifluoro-ethoxy)-6-{[3- (trifluoromethyl)phenyl]amino}-1,3,5- triazin-2-yl]amino}piperidin-1- yl)methanone | 592.9 | C₂₄H₂₀ClF₇N₆O₂ |
| 30 | 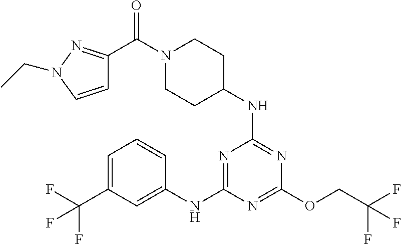 | (1-ethyl-1H-pyrazol-3-yl)(4-{[4-(2,2,2- trifluoro-ethoxy)-6-{[3- (trifluoromethyl)phenyl]amino}-1,3,5- triazin-2-yl]amino}piperidin-1- yl)methanone | 558.5 | C₂₃H₂₄F₆N₈O₂ |
| 31 | 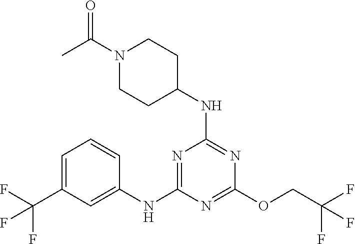 | 1-(4-{[4-(2,2,2-trifluoroethoxy)-6-{[3- (trifluoromethyl)phenyl]amino}-1,3,5- triazin-2-yl]amino}piperidin-1-yl)ethanone | 478.4 | C₁₉H₂₀F₆N₆O₂ |
| 32 | 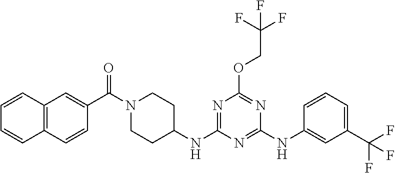 | naphthalen-2-yl(4-{[4-(2,2,2- trifluoroethoxy)-6-{[3- (trifluoromethyl)phenyl]amino}-1,3,5- triazin-2-yl]amino}piperidin-1- yl)methanone | 590.5 | C₂₈H₂₄F₆N₆O₂ |
| 33 | 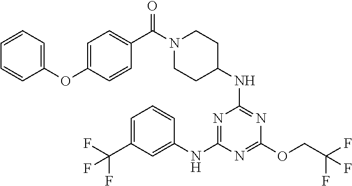 | (4-phenoxyphenyl)(4-{[4-(2,2,2- trifluoroethoxy)-6-{[3- (trifluoromethyl)phenyl]amino}-1,3,5- triazin-2-yl]amino}piperidin-1- yl)methanone | 632.6 | C₃₀H₂₆F₆N₆O₃ |
| 34 | 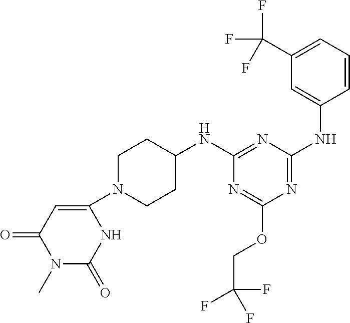 | 3-methyl-6-(4-[(4-(2,2,2-trifluoroethoxy)-6- {[3-(trifluoromethyl)phenyl]amino}-1,3,5- triazin-2-yl)-amino]piperidin-1- yl}pyrimidine-2,4(1H,3H)-dione | 560.5 | C₂₂H₂₂F₆N₈O₃ |
| 35 | 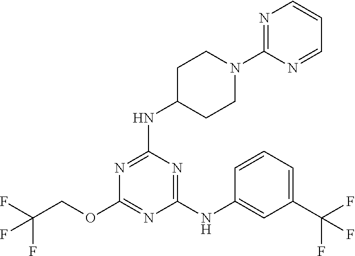 | N2-(1-(pyrimidin-2-yl)piperidin-4-yl)-6- (2,2,2-trifluoroethoxy)-N4-(3- (trifluoromethyl)phenyl)-1,3,5-triazine-2,4- diamine | 514.4 | C₂₁H₂₀F₆N₈O |
| 36 | 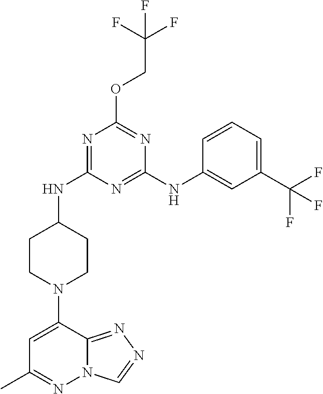 | N-[1-(6-methyl[1,2,4]triazolo[4,3- b]pyridazin-8-yl)piperidin-4-yl]-6-(2,2,2- trifluoroethoxy)-N′-[3- (trifluoromethyl)phenyl]-1,3,5-triazine-2,4- diamine | 568.5 | C₂₃H₂₂F₆N₁₀O |
| 37 | 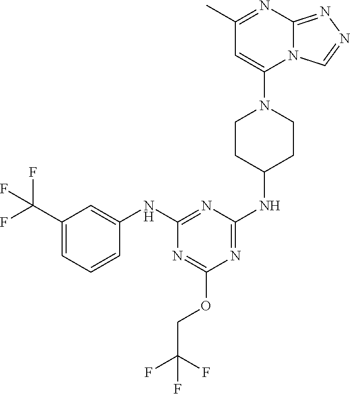 | N2-(1-(7-methyl-[1,2,4]triazolo[4,3- a]pyrimidin-5-yl)piperidin-4-yl)-6-(2,2,2- trifluoroethoxy)-N4-(3- (trifluoromethyl)phenyl)-1,3,5-triazine-2,4- diamine | 568.5 | C₂₃H₂₂F₆N₁₀O |
| 38 | 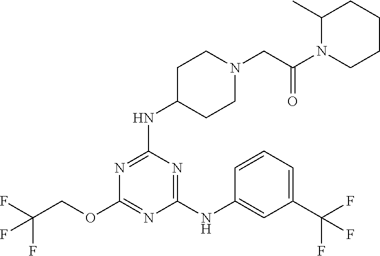 | 1-(2-methylpiperidin-1-yl)-2-(4-{[4-(2,2,2- trifluoro-ethoxy)-6-{[3- (trifluoromethyl)phenyl]-amino}-1,3,5- triazin-2-yl]amino}piperidin-1-yl)ethanone | 575.6 | C₂₅H₃₁F₆N₇O₂ |
| 39 | 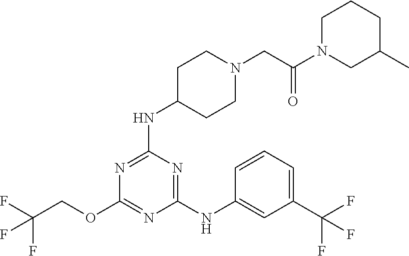 | 1-(3-methylpiperidin-1-yl)-2-(4-(4-(2,2,2- trifluoroethoxy)-6-(3- (trifluoromethyl)phenylamino)-1,3,5- triazin-2-ylamino)piperidin-1-yl)ethanone | 575.6 | C₂₅H₃₁F₆N₇O₂ |
| 40 | 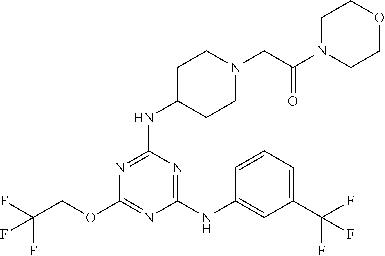 | 1-morpholino-2-(4-(4-(2,2,2- trifluoroethoxy)-6-(3- (trifluoromethyl)phenylamino)-1,3,5- triazin-2-ylamino)piperidin-1-yl)ethanone | 563.5 | C₂₃H₂₇F₆N₇O₃ |
| 41 | 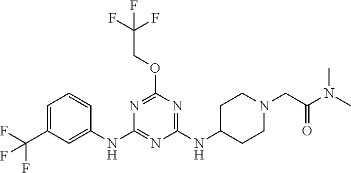 | N,N-dimethyl-2-(4-(4-(2,2,2- trifluoroethoxy)-6-(3- (trifluoromethyl)phenylamino)-1,3,5- triazin-2-ylamino)piperidin-1-yl)acetamide | 521.5 | C₂₁H₂₅F₆N₇O₂ |
| 42 | 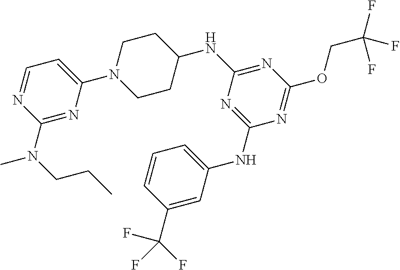 | N-(1-{2-[methyl(propyl)amino]pyrimidin-4- yl}piperidin-4-yl)-6-(2,2,2-trifluoroethoxy)- N′-[3-(trifluoromethyl)phenyl]-1,3,5- triazine-2,4-diamine | 585.6 | C₂₅H₂₉F₆N₉O |
| 43 | 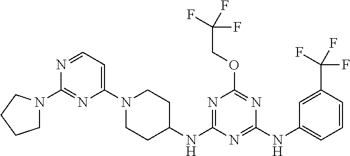 | N-[1-(2-pyrrolidin-1-ylpyrimidin-4- yl)piperidin-4-yl]-6-(2,2,2-trifluoroethoxy)- N′-[3-(trifluoromethyl)phenyl]-1,3,5- triazine-2,4-diamine | 583.5 | C₂₅H₂₇F₆N₉O |
| 44 | 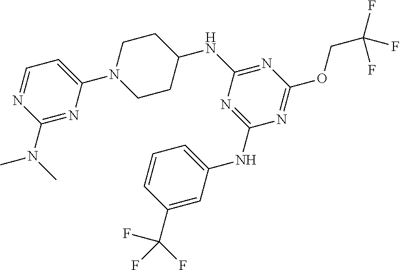 | N-{1-[2-(dimethylamino)pyrimidin-4- yl]piperidin-4-yl}-6-(2,2,2-trifluoroethoxy)- N′-[3-(trifluoromethyl)phenyl]-1,3,5- triazine-2,4-diamine | 557.5 | C₂₃H₂₅F₆N₉O |
| 45 | 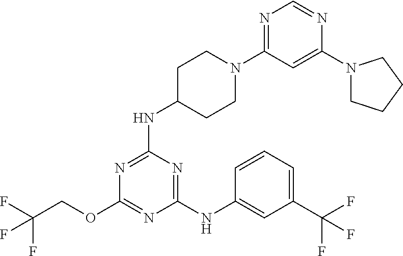 | N2-(1-(6-(pyrrolidin-1-yl)pyrimidin-4- yl)piperidin-4-yl)-6-(2,2,2-trifluoroethoxy)- N4-(3-(trifluoromethyl)phenyl)-1,3,5- triazine-2,4-diamine | 583.5 | C₂₅H₂₇F₆N₉O |
| 46 | 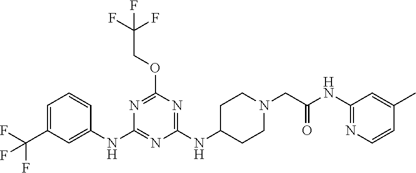 | N-(4-methylpyridin-2-yl)-2-(4-(4-(2,2,2- trifluoroethoxy)-6-(3- (trifluoromethyl)phenylamino)-1,3,5- triazin-2-ylamino)piperidin-1-yl)acetamide | 584.5 | C₂₅H₂₆F₆N₈O₂ |
| 47 | 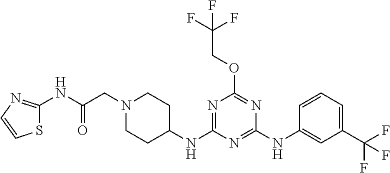 | N-1,3-thiazol-2-yl-2-{4-[(4-(2,2,2- trifluoroethoxy)-6-{[3- (trifluoromethyl)phenyl]amino}-1,3,5- triazin-2-yl)amino]piperidin-1-yl}acetamide | 576.5 | C₂₂H₂₂F₆N₈O₂S |
| 48 | 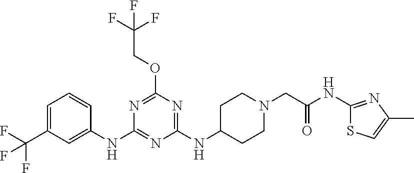 | N-(4-methylpyridin-2-yl)-2-(4-(4-(2,2,2- trifluoroethoxy)-6-(3- (trifluoromethyl)phenylamino)-1,3,5- triazin-2-ylamino)piperidin-1-yl)acetamide | 590.5 | C₂₃H₂₄F₆N₈O₂S |
| 49 | 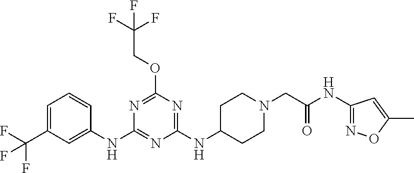 | N-(5-methylisoxazol-3-yl)-2-(4-(4-(2,2,2- trifluoroethoxy)-6-(3- (trifluoromethyl)phenylamino)-1,3,5- triazin-2-ylamino)piperidin-1-yl)acetamide | 574.5 | C₂₃H₂₄F₆N₈O₃ |
| 50 | 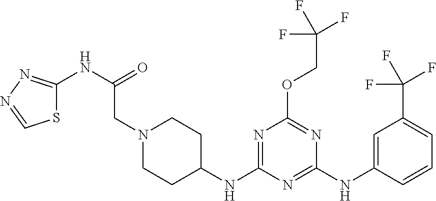 | N-1,3,4-thiadiazol-2-yl-2-{4-[(4-(2,2,2- trifluoro-ethoxy)-6-{[3- (trifluoromethyl)phenyl]amino}-1,3,5- triazin-2-yl)amino]piperidin-1-yl}acetamide | 577.5 | C21H21F6N9O2S |
| 51 | 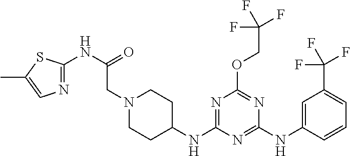 | N-(5-methyl-1,3-thiazol-2-yl)-2-{4-[(4- (2,2,2-tri-fluoroethoxy)-6-{[3- (trifluoromethyl)phenyl]amino}-1,3,5- triazin-2-yl)amino]piperidin-1-yl}acetamide | 590.6 | C23H24F6N8O2S |
| 52 | 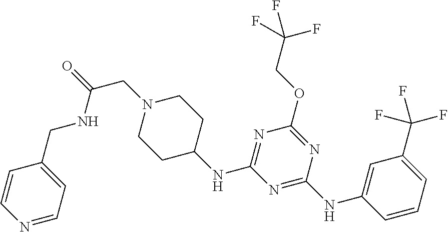 | N-(pyridin-4-ylmethyl)-2-{4-[(4-(2,2,2- trifluoro-ethoxy)-6-{[3- (trifluoromethyl)phenyl]amino}-1,3,5- triazin-2-yl)amino]piperidin-1-yl}acetamide | 584.5 | C25H26F6N8O2 |
| 53 | 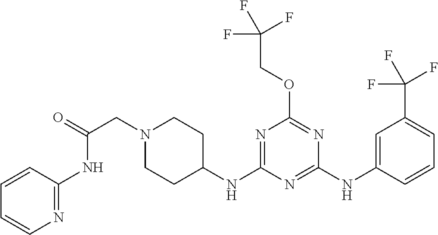 | N-pyridin-2-yl-2-{4-[(4-(2,2,2- trifluoroethoxy)-6-{[3- (trifluoromethyl)phenyl]amino}-1,3,5- triazin-2-yl)amino]piperidin-1-yl}acetamide | 570.5 | C24H24F6N8O2 |
| 54 | 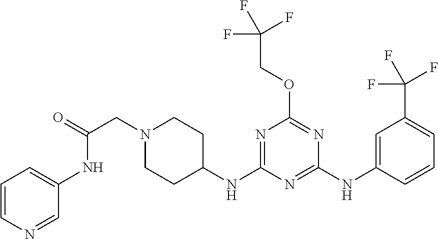 | N-pyridin-3-yl-2-{4-[(4-(2,2,2- trifluoroethoxy)-6-{[3- (trifluoromethyl)phenyl]amino}-1,3,5- triazin-2-yl)amino]piperidin-1-yl}acetamide | 570.5 | C24H24F6N8O2 |
| 55 | 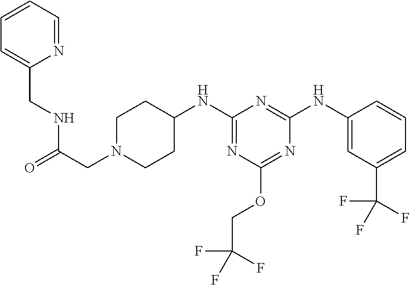 | N-(pyridin-2-ylmethyl)-2-{4-[(4-(2,2,2- trifluoro-ethoxy)-6-{[3- (trifluoromethyl)phenyl]amino}-1,3,5- triazin-2-yl)amino]piperidin-1-yl}acetamide | 584.5 | C25H26F6N8O2 |
| 56 | 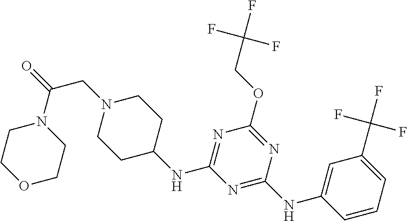 | N-morpholin-4-yl-2-{4-[(4-(2,2,2- trifluoroethoxy)-6-{[3- (trifluoromethyl)phenyl]amino}-1,3,5- triazin-2-yl)amino}piperidin-1-yl}acetamide | 563.5 | C23H27F6N7O3 |
| 57 | 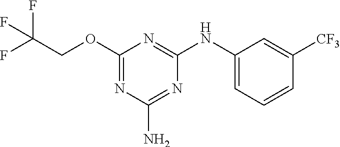 | 6-(2,2,2-Trifluoro-ethoxy)-N-(3- trifluoromethyl-phenyl)-[1,3,5]triazine-2,4- diamine | 353 | C12H9F6N5O |

### Procedures and Analytical Data for Table 36.

Entries 2 to 33 were prepared by the methods associated with Library 36a. Entries 38 to 41 and entries 46 to 56 were prepared by the methods corresponding to Library 36b. Entries 34 to 37 and 42 to 45 were prepared by the procedures described for Library 36c.

### 1. (4-Dimethylamino-phenyl)-{4-[4-(2,2,2-trifluoro-ethoxy)-6-(3-trifluoromethyl-phenylamino)-[1,3,5]triazin-2-ylamino]-piperidin-1-yl}-methanone

A mixture of compound a (300 mg, 0.69 mmol), 4-dimethylamino-benzoic acid (b) (125 mg, 0.76 mmol), TBTU (254 mg, 0.79 mmol), NEt₃ (139 mg, 1.37 mmol) in acetonitrile (10 mL) was stirred at 50° C. for 3 hours, cooled down to room temperature, diluted with 40% aqueous K₂CO₃ solution and extracted with ethyl acetate. The combined organic phases were washed with water, dried over sodium sulfate and evaporated. The residue was purified by column chromatography on silica gel (25% ethyl acetate/dichloromethane) and triturated with hexane giving compound c as white crystals. Yield 292 mg, 73%. ¹H-NMR (400 MHz, DMSO-D₆) δ_{H}: 1.50 (2H, m), 1.90 (2H, m), 2.94 (6H, s), 3.02 (2H, m), 4.10 (3H, broad peaks, Z/E forms), 4.96 (2H, broad q, J=7.5 Hz), 6.72 (2H, d, J=8.5 Hz), 7.26 (2H, d, J=8.5 Hz), 7.32 (1H, m), 7.52 (1H, broad t, J=8.5 Hz), 7.61-7.77 (1H, two broad peaks, Z/E forms), 7.82-8.03 (1H, two broad peaks, Z/E forms), 8.08-8.32 (1H, two broad peaks, Z/E forms), 9.70-9.92 (1H, two broad peaks, Z/E forms). MW 583.54. LCMS t_{R} (min): 2.08. MS (APCI+), m/z 584.50, 585.83 [M+H]⁺ HPLC t_{R} (min): 15.77. Mₚ 123-125° C.

### 2. N-[1-(2-chlorobenzyl)piperidin-4-yl]-6-(2,2,2-trifluoroethoxy)-N′-[3-(trifluoromethyl)phenyl]-1,3,5-triazine-2,4-diamine

LCMS: M+1=560.9; NMR 1H, DMSO-d6 δ, ppm: 1.60 m (2H); 1.85 d (2H); 3.10 t (2H); 4.10 d (3H); 4.95 m (2H); 7.30-7.50 m (7H); 7.80-8.20 m (2H); 9.60 bs (1H).

### 3. N-[1-(2,4-difluorobenzyl)piperidin-4-yl]-6-(2,2,2-trifluoroethoxy)-N′-[3-(trifluoromethyl)phenyl]-1,3,5-triazine-2,4-diamine

LCMS: M+1=562.4; NMR 1H, DMSO-d6 δ, ppm: 1.60 m (2H); 1.85 d (2H); 3.10 t (2H); 4.10 d (3H); 4.95 m (2H); 7.30-7.50 m (3H); 7.70-8.20 m (5H); 9.70 bs (1H).

### 4. 6-(2,2,2-trifluoroethoxy)-N-[3-(trifluoromethyl)phenyl]-N′-[1-(3,4,5-trimethoxybenzyl)piperidin-4-yl]-1,3,5-triazine-2,4-diamine

LCMS: M+1=617.5 NMR 1H, DMSO-d6 δ, ppm: 1.60 m (2H); 1.85 d (2H); 2.10 t (2H); 2.82 d (2H); 3.45 s (2H); 3.67 s 3H); 3.83 m (7H); 4.95 m (2H); 6.65 s (2H); 7.31 d (1H); 7.45 m (2H); 8.03 m (2H); 9.75 bs (1H).

### 5. 6-(2,2,2-trifluoroethoxy)-N-{1-[2-(trifluoromethyl)benzyl]piperidin-4-yl}-N′-[3-(trifluoromethyl)phenyl]-1,3,5-triazine-2,4-diamine

LCMS: M+1=594.4; NMR 1H, DMSO-d6 δ, ppm: 1.60 m (2H); 1.85 d (2H); 3.10 t (2H); 4.10 d (3H); 4.95 m (2H); 7.25-7.45 m (2H); 7.50-7.60 m (3H); 7.80-8.00 m (3H); 8.20 d (1H); 9.55 bs (1H).

### 6. 6-(2,2,2-trifluoroethoxy)-N-{1-[3-(trifluoromethyl)benzyl]piperidin-4-yl}-N′-[3-(trifluoromethyl)phenyl]-1,3,5-triazine-2,4-diamine

LCMS: M+1=594.4; NMR 1H, DMSO-d6 δ, ppm: 3.60 t (4H); 3.90 t (4H); 4.95 m (2H); 7.25 d (1H); 7.35 d (1H); 7.50 m (2H); 7.75-7.95 m (3H); 8.20 s (1H); 8.70 s (1H); 9.80 bs (1H).

### 7. N-(3-fluorophenyl)-4-{[4-(2,2,2-trifluoroethoxy)-6-{[3-(trifluoromethyl)phenyl]amino}-1,3,5-triazin-2-yl]amino}piperidine-1-carboxamide

LCMS: M+1=573.4; NMR 1H, DMSO-d6 δ ppm: 3.60 t (4H); 3.90 t (4H); 4.20 s (4H); 4.95 m (2H); 6.70-6.90 m (2H); 7.00 s (1H); 7.35 d (1H); 7.50 t (1H); 7.80 d (1H); 8.20 t (2H); 9.80 bs (1H).

### 8. N-benzyl-4-{[4-(2,2,2-trifluoroethoxy)-6-{[3-(trifluoromethyl)phenyl]amino}-1,3,5-triazin-2-yl]amino}piperidine-1-carboxamide

LCMS: M+1=569.5; NMR 1H, DMSO-d6 δ, ppm: 3.60 t (4H); 3.80 t (4H); 4.00 m (1H); 4.95 m (2H); 7.20 t (1H); 7.30 d (1H); 7.50 m (2H); 7.75 d (1H); 7.90 d (1H); 8.25 s (1H); 8.60 s (1H); 9.80 bs (1H).

### 9. N-(2-phenylethyl)-4-{[4-(2,2,2-trifluoroethoxy)-6-{[3-(trifluoromethyl)phenyl]amino}-1,3,5-triazin-2-yl]amino}piperidine-1-carboxamide

LCMS: M+1=583.5; NMR 1H, DMSO-d6 δ, ppm: 3.60 t (4H); 3.90 t (4H); 4.95 m (2H); 7.30 d (1H); 7.50-7.60 m (2H); 7.90 m (2H); 8.20 s (1H); 8.50 s (1H); 9.80 bs (1H).

### 10. N-cyclohexyl-4-{[4-(2,2,2-trifluoroethoxy)-6-{[3-(trifluoromethyl)phenyl]amino}-1,3,5-triazin-2-yl]amino}piperidine-1-carboxamide

LCMS: M+1=562.5 NMR 1H, DMSO-d6 δ, ppm: 1.45 m (14H); 2.75 t (2H); 3.48 bm (1H); 3.93 d (3H); 4.95 m (2H); 5.82 d (1H); 7.32 d (1H); 7.53 t (2H); 8.08 bm (2H); 9.68 bs (1H).

### 11. N-(3-chloro-4-fluorophenyl)-4-{[4-(2,2,2-trifluoroethoxy)-6-{[3-(trifluoromethyl)phenyl]amino}-1,3,5-triazin-2-yl]amino}piperidine-1-carboxamide

LCMS: M+1=607.9; NMR 1H, DMSO-d6 δ, ppm: 3.60 t (4H); 3.90 m (10H); 4.95 m (2H); 7.00 m (3H); 7.30 d (1H); 7.50 t (1H); 7.80 d (1H); 8.20 s (1H); 9.80 bs (1H).

### 12. N-cyclopentyl-4-{[4-(2,2,2-trifluoroethoxy)-6-{[3-(trifluoromethyl)phenyl]amino}-1,3,5-triazin-2-yl]amino}piperidine-1-carboxamide

LCMS: M+1=547.5; NMR 1H, DMSO-d6 δ, ppm: 3.60 t (4H); 3.90 t (4H); 4.95 m (2H); 7.00 m (3H); 7.30 d (1H); 7.50 m (2H); 7.80 m (2H); 8.20 s (1H); 8.70 s (2H); 9.80 bs (1H).

### 13. (4-chlorophenyl)(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=574.9; NMR 1H, DMSO-d6 δ, ppm: 1.20-1.40 m (5H); 1.60-1.80 m (5H); 2.65 m (1H); 3.60 t (4H); 3.80 t (4H); 4.95 m (2H); 7.30 d (1H); 7.50 t (1H); 7.80 d (1H); 8.20 s (1H); 9.80 bs (1H).

### 14. (6-chloropyridin-3-yl)(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=575.9; NMR 1H, DMSO-d6 δ, ppm: 1.20 t (2H); 1.50-1.80 m (5H); 2.15 m (1H); 2.40 d (2H); 3.60 t (4H); 3.80 t (4H); 4.95 m (2H); 7.30 d (1H); 7.50 t (1H); 7.80 d (1H); 8.20 s (1H); 9.80 bs (1H).

### 15. (3-chlorophenyl)(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=574.9; NMR 1H, DMSO-d6 δ, ppm: 3.30 t (4H); 3.90 t (4H); 4.47 s (2H); 4.95 m (2H); 7.30 m (6H); 7.50 t (1H); 7.80 d (1H); 8.20 s (1H); 9.80 bs (1H).

### 16. (4-chloro-3-nitrophenyl)(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=619.9; NMR 1H, DMSO-d6 δ, ppm: 3.30 t (4H); 3.90 t (4H); 4.95 m (2H); 7.30 d (1H); 7.50 t (1H); 7.60 m (2H); 7.80 m (2H); 8.20 s (1H); 9.80 bs (1H).

### 17. 4-[(4-{[4-(2,2,2-trifluoroethoxy)-6-{[3-(trifluoromethyl)phenyl]amino}-1,3,5-triazin-2-yl]amino}piperidin-1-yl)carbonyl]benzonitrile

LCMS: M+1=565.4; NMR 1H, DMSO-d6 δ, ppm: 2.35 s (6H); 3.10 t (4H); 3.90 t (4H); 4.95 m (2H); 7.30 d (1H); 7.50 t (1H); 7.80 d (1H); 8.20 s (1H); 9.80 bs (1H).

### 18. cyclopentyl(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=532.4; 1H NMR (DMSO-d6, 90° C., ppm): d=1.66 m (12H), 3.00 m (3H), 4.12 m (3H), 4.92 q (2H), 7.38 m (2H), 7.52 t (1H), 7.92 s (1H), 8.18 s (1H), 9.52 s (1H).

### 19. cyclopropyl(4-{[4-(2,2,2-trifluoroethoxy)-6-{[3-(trifluoromethyl)phenyl]amino}-1,3,5-triazin-2-yl]amino}piperidin-1-yl)methanone

1H NMR (DMSO-d6, 90° C., ppm): d=0.74 m (4H, 2CH2), 1.50 q (2H, CH2), 1.94 t (3H, CH), 3.02 t (2H, CH2), 4.09 m (1H, CH), 4.25 d (2H, CH), 4.94 q (2H, CH2), 7.32 d (1H, Ar), 7.42 s (1H, CH), 7.52 t (1H, CH), 7.93 s (1H, Ar), 8.20 s (1H, Ar), 9.58 s (1H, NH). LC-MS [M+1]: calc'd: 504.4; obs'd: 505.5.

### 20. (3,4-dimethoxyphenyl)(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=600.5; 1H NMR (DMSO-d6, 90° C., ppm): d=1.56 q (2H), 1.94 d (2H), 3.08 t (8H), 3.86 s (6H), 4.10 d (3H), 4.92 q (2H), 7.00 m (3H), 7.34 d (1H), 7.50 m (2H), 8.04 m (2H), 9.62 s (1H).

### 21. 2-(3,4-dimethoxyphenyl)-1-(4-{[4-(2,2,2-trifluoroethoxy)-6-{[3-(trifluoromethyl)phenyl]amino}-1,3,5-triazin-2-yl]amino}piperidin-1-yl)ethanone

LCMS: M+1=614.5; 1H NMR (DMSO-d6, 90° C., ppm): d=1.38 m (2H), 1.86 d (2H), 3.72 m (8H), 4.12 m (3H), 4.92 q (2H), 6.86 m (3H), 7.42 m (3H), 8.06 m (2H), 9.62 s (1H).

### 22. 2-(4-fluorophenyl)-1-(4-{[4-(2,2,2-trifluoroethoxy)-6-{[3-(trifluoromethyl)phenyl]amino}-1,3,5-triazin-2-yl]amino}piperidin-1-yl)ethanone

LCMS: M+1=572.4; 1H NMR (DMSO-d6, 90° C., ppm): d=1.40 q (2H), 1.88 d (2H), 3.74 s (2H), 4.08 m (3H), 4.92 q (2H), 7.12 t (2H), 7.40 m (5H), 8.06 m (2H), 9.62 s (1H).

### 23. pyridin-3-yl(4-{[4-(2,2,2-trifluoroethoxy)-6-{[3-(trifluoromethyl)phenyl]amino}-1,3,5-triazin-2-yl]amino}piperidin-1-yl)methanone

H NMR (DMSO-d6, 90° C., ppm): d=1.58 q (2H, CH2), 1.96 d (2H, CH2), 3.16 t (3H, CH2), 4.06 d (3H, CH), 4.96 m (2H, CH2), 7.34 d (1H, Ar), 7.50 m (3H, Ar), 7.82 d (1H, Ar), 8.08 d (2H, Ar), 8.64 t (2HAr), 9.64 s (1H, NH). LC-MS [M+1]: calc'd: 541.4; obs'd: 542.2.

### 24. cyclohexyl(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=546.5; 1H NMR (DMSO-d6, 90° C., ppm): d=1.60 m (15H), 2.58 t (2H), 4.12 m (3H), 4.92 q (2H), 7.42 m (3H), 8.06 m (2H), 9.62 s (1H).

### 25. 2-cyclopentyl-1-(4-{[4-(2,2,2-trifluoroethoxy)-6-{[3-(trifluoromethyl)phenyl]amino}-1,3,5-triazin-2-yl]amino}piperidin-1-yl)ethanone

1H NMR (DMSO-d6, 90° C., ppm): d=1.18 m (2H, CH2), 1.54 m (6H, 3CH2), 1.77 q (2H, CH2), 1.92 d (2H, CH2), 2.19 m (1H, CH), 2.34 d (2H, CH2), 4.07 m (3H, CH), 4.95 q (2H, CH2), 7.32 d (1H, Ar), 7.44 s (1H, Ar), 7.52 t (1H, Ar), 7.93 s (1H, Ar), 8.20 s (1H, Ar), 9.62 s (1H, NH). LC-MS [M+1]: calc'd: 546.5; obs'd: 547.4.

### 26. 2-(phenylamino)-1-(4-{[4-(2,2,2-trifluoroethoxy)-6-{[3-(trifluoromethyl)phenyl]amino}-1,3,5-triazin-2-yl]amino}piperidin-1-yl)ethanone

1H NMR (DMSO-d6, 90° C., ppm): d=1.53 m (2H, CH2), 1.94 d (2H, CH2), 2.95 m (5H, from 3 CH2), 3.91 s (2H, CH2), 4.10 m (3H, from 2 CH2), 4.94 q (2H, CH2), 5.26 m (1H, CH), 6.58 t (1H, Ar), 6.66 d (1H, Ar), 7.09 t (1H, Ar), 7.32 d (1H, Ar), 7.50 m (2H, Ar), 7.92 s br. (1H, NH), 8.19 s br. (1H, NH), 9.60 s br. (1H, NH). LC-MS [M+1]: calc'd: 570.5; obs'd: 570.4.

### 27. (3,4-difluorophenyl)(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=576.4; 1H NMR (DMSO-d6, 90° C., ppm): d=1.54 q (2H), 2.94 d (2H), 3.12 t (2H), 4.08 m (3H), 4.92 q (2H), 7.38 m (6H), 8.12 m (2H), 9.62 s (1H).

### 28. 3-(3,5-dimethyl-1H-pyrazol-1-yl)-1-(4-{[4-(2,2,2-trifluoroethoxy)-6-{[3-(trifluoromethyl)phenyl]amino}-1,3,5-triazin-2-yl]amino}piperidin-1-yl)propan-1-one

LCMS: M+1=586.5; 1H NMR (DMSO-d6, 90° C., ppm): 1=1.49 m (2H), 1.98 m (2H), 2.09 s (3H), 2.22 s (3H), 2.89 m (5H), 4.05 m (4H), 4.99 m (2H), 5.76 s (1H), 7.42 m (3H), 8.28 d (2H), 9.62 s (1H).

### 29. (3-chloro-4-fluorophenyl)(4-{[4-(2,2,2-trifluoroethoxy)-6-{[3-(trifluoromethyl)phenyl]amino}-1,3,5-triazin-2-yl]amino}piperidin-1-yl)methanone

1H NMR (DMSO-d6, 90° C., ppm): d=1.58 q (2H, CH2), 1.94 d (2H, CH2), 3.12 t (3H, CH2), 3.98 d (2H, CH2), 4.12 m (1H, CH), 4.96 q (2H, CH2), 7.50 m (6H, Ar), 7.96 s (1H, Ar), 8.20 s (1H, Ar), 9.64 s (1H, NH). LC-MS [M+1]: calc'd: 592.9; obs'd: 593.2.

### 30. (1-ethyl-1H-pyrazol-3-yl)(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=558.4; 1H NMR (DMSO-d6, 80° C., ppm): 1=1.39 t (3H), 1.52q (2H), 1.98 m (2H), 3.12 m (2H), 4.15 m (3H), 4.50 d (2H), 4.97 m (2H), 6.50 s (1H), 7.48 m (3H), 7.71 s (1H), 8.01 m (2H), 9.63 s (1H).

### 31. 1-(4-{[4-(2,2,2-trifluoroethoxy)-6-{[3-(trifluoromethyl)phenyl]amino}-1,3,5-triazin-2-yl]amino}piperidin-1-yl)ethanone

LCMS: M+1=479; 1H NMR, DMSO-d6 δ, ppm: 1.48 m (2H); 1.90 m (2H); 2.01 s (3H); 3.05 bs (2H); 4.08 bs (3H); 4.95 m (2H); 7.32 d (1H); 7.45 m (3H); 7.95 bs (1H); 8.22 bs (1H); 9.64 bs (1H).

### 32. naphthalen-2-yl(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=591; 1H NMR, DMSO-d6 δ, ppm: 1.62 m (2H); 1.98 m (2H); 3.04 m (2H); 4.04 bs (3H); 4.98 m (2H); 7.04 m (1H); 7.52 m (5H); 7.92 m (5H); 8.04 bs (1H); 9.62 bs (1H).

### 33. (4-phenoxyphenyl)(4-{[4-(2,2,2-trifluoroethoxy)-6-{[3-(trifluoromethyl)phenyl]amino}-1,3,5-triazin-2-yl]amino}piperidin-1-yl)methanone

LCMS: M+1=633; 1H NMR, DMSO-d6 δ, ppm: 1.65 m (2H); 1.87 m (2H); 3.25 m (2H); 4.18 m (3H); 4.95 m (2H); 7.32 m (12H); 7.92 d (1H); 8.17 bs (1H); 9.39 bs (1H).

### 34. 3-methyl-6-{4-[(4-(2,2,2-trifluoroethoxy)-6-{[3-(trifluoromethyl)phenyl]amino}-1,3,5-triazin-2-yl)amino]piperidin-1-yl}pyrimidine-2,4(1H,3H)-dione

LCMS: M+1=561.0; 1H NMR, DMSO-d6 δ, ppm: 1.6 m (2H); 1.95 m (2H); 3.10 s (3H); 3.75 m (2H); 4.05 m (1H); 4.80 s (1H); 4.95 m (2H); 7.30-7.50 m (3H); 7.80-8.30 m (2H); 9.60 s (1H); 10.2 s (1H).

### 35. N-2-(1-(pyrimidin-2-yl)piperidin-4-yl)-6-(2,2,2-trifluoroethoxy)-N-4-(3-(trifluoromethyl)phenyl)-1,3,5-triazine-2,4-diamine

LCMS: M+1=515.5; 1H NMR, DMSO-d6 δ, ppm: 1.55 m (2H); 1.95 d (2H); 3.10 t (2H); 4.15 m (1H); 4.60 d (2H); 4.95 m (2H); 6.60 t (1H); 7.26-7.56 m (3H); 7.84-8.38 m (4H); 9.60 s (1H).

### 36. N-[1-(6-methyl[1,2,4]triazolo[4,3-b]pyridazin-8-yl)-piperidin-4-yl]-6-(2,2,2-trifluoroethoxy)-N′-[3-(trifluoromethyl)phenyl]-1,3,5-triazine-2,4-diamine

LCMS: M+1=569.6; 1H NMR, DMSO-d6 δ, ppm: 1.75 m (2H); 2.01 m (2H); 2.40 (3H); 3.40 t (2H); 4.20 m (1H); 4.70-5.00 m (4H); 6.20 s (1H); 7.20-7.60 m (3H); 7.90-8.20 m (2H); 9.15 s (1H); 9.60 s (1H).

### 37. N-2-(1-(7-methyl-[1,2,4]triazolo[4,3-a]pyrimidin-5-yl)piperidin-4-yl)-6-(2,2,2-trifluoroethoxy)-N-4-(3-(trifluoromethyl)phenyl)-1,3,5-triazine-2,4-diamine

LCMS: M+1=569.5; 1H NMR, DMSO-d6 δ, ppm: 1.8 m (2H); 1.95 m (2H); 3.35 m (2H); 4.20 m (1H); 4.55 m (2H); 4.95 m (2H); 6.50 s (1H); 7.20-7.60 m (3H); 7.80-8.40 m (3H); 9.60 s (1H).

### 38. 1-(2-methylpiperidin-1-yl)-2-(4-{[4-(2,2,2-trifluoroethoxy)-6-{[3-(trifluoromethyl)phenyl]amino}-1,3,5-triazin-2-yl]amino}piperidin-1-yl)ethanone

LCMS: M+1=576.6; 1H NMR, DMSO-d6 δ, ppm: 1.12 d (3H); 1.32 m (1H); 1.60 m (7H); 1.88 d (2H); 2.15 t (2H); 2.82 m (3H); 3.15 m (2H) 3.80 bs (1H); 4.07 d (1H); 4.52 s (1H); 4.92 m (2H); 7.25-7.55 m (3H); 7.80-8.20 m (2H); 9.55 bs (1H).

### 39. 1-(3-methylpiperidin-1-yl)-2-(4-(4-(2,2,2-trifluoroethoxy)-6-(3-(trifluoromethyl)phenylamino)-1,3,5-triazin-2-ylamino)piperidin-1-yl)ethanone

LCMS: M+1=576.6; 1H NMR, DMSO-d6 δ, ppm: 0.9 d (3H); 1.00-1.90 m (10H); 2.18 t (2H); 2.70-3.20 m (5H); 3.92 m (3H); 4.92 m (2H); 7.25-7.55 m (3H); 7.80-8.20 m (2H); 9.55 bs (1H).

### 40. 1-morpholino-2-(4-(4-(2,2,2-trifluoroethoxy)-6-(3-(trifluoromethyl)phenylamino)-1,3,5-triazin-2-ylamino)piperidin-1-yl)ethanone

LCMS: M+1=564.6; 1H NMR, DMSO-d6 δ, ppm: 1.60 m (2H); 1.85 d (2H); 2.18 t (2H); 2.85 d (2H); 3.18 s (2H); 3.58 m (8H); 3.82 m (1H); 4.92 m (2H); 7.30 d (2H); 7.50 t (1H); 7.80-8.20 m (2H); 9.55 bs (1H).

### 41. N,N-dimethyl-2-(4-(4-(2,2,2-trifluoroethoxy)-6-(3-(trifluoromethyl)phenylamino)-1,3,5-triazin-2-ylamino)piperidin-1-yl)acetamide

LCMS: M+1=548.6; 1H NMR, DMSO-d6 δ, ppm: 1.60 m (2H); 1.85 d (2H); 2.24 t (2H); 2.82 m (2H); 2.90 s (6H); 3.12 s (2H); 3.88 bs (1H); 4.92 m (2H); 7.30 d (2H); 7.50 t (1H); 7.90-8.20 m (2H); 9.55 bs (1H).

### 42. N-{1-[2-(dimethylamino)pyrimidin-4-yl]piperidin-4-yl}-6-(2,2,2-trifluoroethoxy)-N′-[3-(trifluoromethyl)phenyl]-1,3,5-triazine-2,4-diamine

LCMS: M+1=586.6; 1H NMR, DMSO-d6 δ, ppm: 0.85 t (3H); 1.55 m (4H); 1.99 d (2H); 3.09 m (5H); 3.51 t (2H); 4.11 m (2H); 4.29 d (1H); 4.98 q (2H); 6.00 d (1H); 7.44 m (3H); 8.12 m (3H); 9.62 s (1H).

### 43. N-[1-(2-pyrrolidin-1-ylpyrimidin-4-yl)piperidin-4-yl]-6-(2,2,2-trifluoroethoxy)-N′-[3-(trifluoromethyl)phenyl]-1,3,5-triazine-2,4-diamine

LCMS: M+1=584.4; 1H NMR, DMSO-d6 δ, ppm: 1.55 m (2H); 1.92 m (6H); 3.05 m (2H); 3.55 m (4H); 4.09 m (2H); 4.23 m (1H); 4.98 q (2H); 6.01 d (1H); 7.41 m (3H); 8.01 m (3H); 9.67 s (1H).

### 44. N-{1-[2-(dimethylamino)pyrimidin-4-yl]piperidin-4-yl}-6-(2,2,2-trifluoroethoxy)-N′-[3-(trifluoromethyl)phenyl]-1,3,5-triazine-2,4-diamine

LCMS: M+1=558.6; 1H NMR, DMSO-d6 δ, ppm: 1.55 m (2H); 1.99 m (2H); 3.11 m (8H); 4.12 m (2H); 4.30 d (1H); 4.97 q (2H); 6.01 d (1H); 7.45 m (3H); 8.10 m (3H); 9.62 (1H).

### 45. N-2-(1-(6-(pyrrolidin-1-yl)pyrimidin-4-yl)piperidin-4-O-6-(2,2,2-trifluoroethoxy)-N-4-(3-(trifluoromethyl)phenyl)-1,3,5-triazine-2,4-diamine

LCMS: M+1=584.5; 1H NMR, DMSO-d6 δ, ppm: 1.60 m (2H); 2.00 m (6H); 3.00-3.60 m (9H); 4.00-4.40 m (3H); 4.95 m (2H); 5.70 s (1H); 6.60 t (1H); 7.25-7.60 m (3H); 7.85-8.20 m (3H); 9.60 s (1H).

### 46. N-(4-methylpyridin-2-yl)-2-(4-(4-(2,2,2-trifluoroethoxy)-6-(3-(trifluoromethyl)phenylamino)-1,3,5-triazin-2-ylamino)piperidin-1-yl)acetamide

LCMS: M+1=585.6; 1H NMR, DMSO-d6 δ, ppm: 1.65 m (2H); 1.95 m (2H); 2.35 m (5H); 2.82 m (2H); 3.18 s (2H); 3.88 bs (1H); 4.92 m (2H); 6.95 d (2H); 7.28 m (2H); 7.50 t (1H); 7.90-8.20 m (4H); 9.60 bs (1H).

### 47. N-1,3-thiazol-2-yl-2-{4-[(4-(2,2,2-trifluoroethoxy)-6-{[3-(trifluoromethyl)phenyl]amino}-1,3,5-triazin-2-yl)amino]piperidin-1-yl}acetamide

LCMS: M+1=577.6; 1H NMR, DMSO-d6 δ, ppm: 1.65 m (2H); 1.90 m (2H); 2.35 t (2H); 2.82 m (2H); 3.28 s (2H); 3.88 bs (1H); 4.92 m (2H); 7.18 d (1H); 7.33 m (2H); 7.44 d (1H); 7.52 t (1H); 7.90-8.20 m (2H); 9.60 bs (1H).

### 48. N-(4-methylpyridin-2-yl)-2-(4-(4-(2,2,2-trifluoroethoxy)-6-(3-(trifluoromethyl)phenylamino)-1,3,5-triazin-2-ylamino)piperidin-1-yl)acetamide

LCMS: M+1=591.6; 1H NMR, DMSO-d6 δ, ppm: 1.65 m (2H); 1.90 m (2H); 2.35 m (5H); 2.82 m (2H); 3.28 s (2H); 3.88 bs (1H); 4.92 m (2H); 6.70 s (1H); 7.33 m (2H); 7.50 t (1H); 7.90-8.20 m (2H); 9.60 bs (1H).

### 49. N-(5-methylisoxazol-3-yl)-2-(4-(4-(2,2,2-trifluoroethoxy)-6-(3-(trifluoromethyl)phenylamino)-1,3,5-triazin-2-ylamino)piperidin-1-yl)acetamide

LCMS: M+1=575.6; 1H NMR, DMSO-d6 δ, ppm: 1.65 m (2H); 1.90 m (2H); 2.35 m (5H); 2.82 m (2H); 3.18 s (2H); 3.88 bs (1H); 4.92 m (2H); 6.65 s (1H); 7.33 m (2H); 7.50 t (1H); 7.90-8.20 m (2H); 9.60 bs (1H). 10.00 bs (1H).

### 50. N-1,3,4-thiadiazol-2-yl-2-{4-[(4-(2,2,2-trifluoroethoxy)-6-{[3-(trifluoromethyl)phenyl]amino}-1,3,5-triazin-2-yl)amino]piperidin-1-yl}acetamide

LCMS: M+1=578.6; 1H NMR, DMSO-d6 δ, ppm: 1.65 m (2H); 1.90 m (2H); 2.35 t (2H); 2.82 m (2H); 3.38 s (2H); 3.88 bs (1H); 4.92 m (2H); 7.25 m (2H); 7.50 t (1H); 7.90-8.20 m (2H); 9.10 s (1H); 9.60 bs (1H).

### 51. N-(5-methyl-1,3-thiazol-2-yl)-2-{4-[(4-(2,2,2-trifluoroethoxy)-6-{[3-(trifluoromethyl)phenyl]amino}-1,3,5-triazin-2-yl)amino]piperidin-1-yl}acetamide

LCMS: M+1=591.6; 1H NMR, DMSO-d6 δ, ppm: 1.65 m (2H); 1.90 m (2H); 2.35 t (2H); 2.50 s (3H); 2.82 m (2H); 3.28 s (2H); 3.88 bs (1H); 4.92 m (2H); 7.10 s (1H); 7.33 m (2H); 7.50 t (1H); 7.90-8.20 m (2H); 9.60 bs (1H).

### 52. N-(pyridin-4-ylmethyl)-2-{4-[(4-(2,2,2-trifluoroethoxy)-6-{[3-(trifluoromethyl)phenyl]amino}-1,3,5-triazin-2-yl)amino]piperidin-1-yl}acetamide

LCMS: M+1=585.6; 1H NMR, DMSO-d6 δ, ppm: 1.65 m (2H); 1.95 m (2H); 2.35 t (2H); 2.82 m (2H); 3.00 s (2H); 3.88 bs (1H); 4.37 d (2H); 4.92 m (2H); 7.25 d (2H); 7.30 m (2H); 7.50 t (1H); 7.90-8.20 m (3H); 8.50 d (2H); 9.60 bs (1H).

### 53. N-pyridin-2-yl-2-{4-[(4-(2,2,2-trifluoroethoxy)-6-{[3-(trifluoromethyl)phenyl]amino}-1,3,5-triazin-2-yl)amino]piperidin-1-yl}acetamide

LCMS: M+1=571.6; 1H NMR, DMSO-d6 δ, ppm: 1.65 m (2H); 1.95 m (2H); 2.35 t (2H); 2.82 m (2H); 3.18 s (2H); 3.88 bs (1H); 4.92 m (2H); 7.08 t (1H); 7.33 m (2H); 7.50 t (1H); 7.78 t (1H); 7.85-8.30 m (4H); 9.70 bd (2H).

### 54. N-pyridin-3-yl-2-{4-[(4-(2,2,2-trifluoroethoxy)-6-{[3-(trifluoromethyl)phenyl]amino}-1,3,5-triazin-2-yl)amino]piperidin-1-yl}acetamide

LCMS: M+1=571.6; 1H NMR, DMSO-d6 δ, ppm: 1.65 m (2H); 1.95 m (2H); 2.35 t (2H); 2.82 m (2H); 3.18 s (2H); 3.88 bs (1H); 4.92 m (2H); 7.28 m (2H); 7.50 t (1H); 7.90-8.20 m (4H); 8.78 s (1H); 9.70 bd (2H).

### 55. N-(pyridin-2-ylmethyl)-2-{4-[(4-(2,2,2-trifluoroethoxy)-6-{[3-(trifluoromethyl)phenyl]amino}-1,3,5-triazin-2-yl)amino]piperidin-1-yl}acetamide

LCMS: M+1=585.6; 1H NMR, DMSO-d6 δ, ppm: 1.65 m (2H); 1.95 m (2H); 2.30 t (2H); 2.82 m (2H); 3.00 s (2H); 3.88 bs (1H); 4.37 d (2H); 4.92 m (2H); 7.28 m (4H); 7.50 t (1H); 7.70 t (1H); 7.90-8.20 m (3H); 8.50 d (2H); 9.60 bs (1H).

### 56. N-morpholin-4-yl-2-{4-[(4-(2,2,2-trifluoroethoxy)-6-{[3-(trifluoromethyl)phenyl]amino}-1,3,5-triazin-2-yl)amino]piperidin-1-yl}acetamide

LCMS: M+1=579.6; 1H NMR, DMSO-d6 δ, ppm: 1.65 m (2H); 1.95 m (2H); 2.35 t (2H); 2.82 m (2H); 3.00 t (4H); 3.18 s (2H); 3.28 t (1H); 3.55 t (1H); 3.65 t (4H); 3.88 bs (1H); 4.92 m (2H); 7.25-7.50 m (3H); 7.80-8.50 m (3H); 9.60 m (1H).

### 57. 6-(2,2,2-Trifluoro-ethoxy)-N-(3-trifluoromethyl-phenyl)-[1,3,5]triazine-2,4-diamine

NaH (250 mg, 60% in oil, 6.25 mmol) was added to a solution of 2,2,2-trifluoroethanol (1 mL) in THF (10 mL) at room temperature. The mixture was stirred for 15 minutes at room temperature. Then a solution of compound a (450 mg, 1.55 mmol) in THF (5 mL) was added to the obtained suspension at room temperature. The final reaction mixture was stirred at room temperature for 24 hours and at 45° C. for 0.5 hours, cooled down to room temperature, diluted with water and extracted with dichloromethane. The combined organic phases were dried over K₂CO₃. The solvent was evaporated at reduced pressure; the residue was triturated with hexane and dried to give a final compound. Yield 500 mg, 91%. ¹H-NMR (400 MHz, DMSO-D₆) δ_{H}: 4.91 (2H, broad q, J=7.5 Hz), 7.21 (2H, broad peak, Z/E forms), 7.32 (1H, broad d, J=8.5 Hz), 7.48 (1H, t, J=8.5 Hz), 7.95 (1H, d, J=8.5 Hz), 8.09 (1H, s), 9.79 (1H, broad peak, Z/E forms). MW 353.23. LCMS t_{R} (min): 1.82. MS (APCI+), m/z 354.16 [M+H]⁺. HPLC t_{R} (min): 15.03. Mp 133-135° C.

### Generic Synthesis of Intermediates and Final Compounds for Table 37

There are two approaches to make the table of compounds shown in the following Scheme. One approach is R4-R6-R2 route. In this route, the R6 fragments were either introduced on the second step or by various protuction/deprotection follow by modification of the R6 fragments to get desired compounds via reductive aminations, acylation, sulfonation, alkylation, and arylations, and etc. Another is R6-R4-R2 route described above. An R4-R6-R2 route and procedures to prepare final compounds in the table are following.

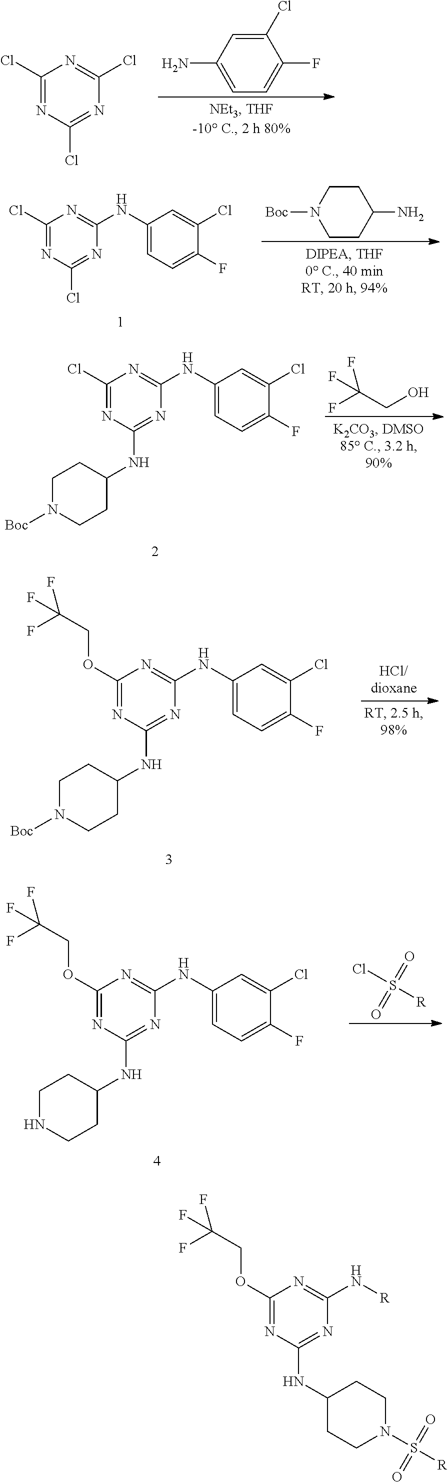

### (3-Chloro-4-fluoro-phenyl)-(4,6-dichloro-[1,3,5]triazin-2-yl)-amine (1)

To a solution of cyanuric chloride (5.530 g, 30 mmol) in THF (40 mL) a solution of 3-chloro-4-fluoro-phenylamine (4.367 g, 30 mmol) and NEt₃ (3.34 g, 33 mmol) in THF (35 mL) was added slowly dropwise at −10° C. The resulting mixture was stirred at −10° C. for 2 hours (TLC control), warmed up to room temperature and diluted with water. The precipitate formed was collected by filtration, washed with water and dried to give compound 1 as white crystalline solid. Yield 7.01 g, 80%. ¹H-NMR (400 MHz, DMSO-D₆) δ_{H}: 7.31-7.41 (1H, two m, Z/E forms), 7.61 (1H, broad peak, Z/E forms), 7.81-7.91 (1H, two broad m, Z/E forms), 10.70-11.20 (1H, broad peaks, Z/E forms). MW 293.52. LCMS t_{R} (min): 1.80. MS (APCI−), m/z 291.29 [M−H]. HPLC t_{R} (min): 15.92 (purity 98.70% (220 nm) 98.65% (254 nm).

### 6-Chloro-N-(3-chloro-4-fluoro-phenyl)-N′-piperidin-4-yl-[1,3,5]triazine-2,4-diamine-N-Boc (2)

To a solution of compound 1 (3.816 g, 13 mmol) in THF (40 mL) a mixture of N-Boc-piperidin-4-ylamine (2.604 g, 13 mmol), DIPEA (1.938 g, 15 mmol) and THF (30 mL) was added at 0° C. The resulting mixture was stirred at 0° C. for 40 minutes, warmed up to room temperature and stirred for 20 hours at room temperature. Then, the mixture was concentrated in vacuum. The residue was diluted with water, filtered and dried to give compound 2 as white crystalline solid. Yield 5.589 g, 94%. ¹H-NMR (400 MHz, DMSO-D₆) δ_{H}: 1.41 (10H, superposition of s (9H) and m (1H)), 1.75 (2H, m), 1.91 (1H, m), 2.95 (1H, m), 3.65 (2H, m), 3.92 (2H, broad peak, Z/E forms), 7.31 (1H, superposition of two m, Z/E forms), 7.51 (1H, broad peak, Z/E forms), 7.65-7.91 (1H, broad peaks, Z/E forms), 8.11-8.25 (1H, broad peaks, Z/E forms), 9.08-10.19 (1H, broad peaks, Z/E forms). MW 457.34. LCMS t_{R} (min): 2.05. MS (APCI+), m/z 456.79, 458.78 [M+H]⁺ (97%).

### N-Boc-N-(3-Chloro-4-fluoro-phenyl)-N′-piperidin-4-yl-6-(2,2,2-trifluoro-ethoxy)-[1,3,5]-triazine-2,4-diamine (3)

A mixture of compound 2 (4.57 g, 10 mmol), 2,2,2-trifluoroethanol (3 g, 30 mmol), powdered K₂CO₃ (2.76 g, 20 mmol) and DMSO (5 mL) was stirred for 3.2 hours at 85° C. When the reaction was over according to TLC, the mixture was cooled down to room temperature and diluted with water. The residue was filtered and washed with water. Purification by column chromatography on silica gel (ethyl acetate/hexane) gave compound C as white crystalline solid. Yield 4.665 g, 90%. ¹H-NMR (400 MHz, DMSO-D₆) δ_{H}: 1.41 (11H, superposition of s (9H) and m (2H)), 1.83 (2H, m), 2.89 (2H, m), 3.95 (3H, broad peak), 4.95 (2H, broad peak), 7.31 (1H, superposition of two m, Z/E forms), 7.51-7.69 (1H, broad peaks, Z/E forms), 7.72 (1H, broad peak, Z/E forms), 8.05-8.19 (1H, broad peaks, Z/E forms), 9.60-9.82 (1H, broad peaks, Z/E forms). MW 520.80. LCMS t_{R} (min): 2.15. MS (APCI+), m/z 520.83, 522.82 [M+H]⁺ (97%).

### N-(3-Chloro-4-fluoro-phenyl)-N′-piperidin-4-yl-6-(2,2,2-trifluoro-ethoxy)-[1,3,5]-triazine-2,4-diamine hydrochloride (4)

Compound 3 (1.86 g, 3.57 mmol) was dissolved in 12% HCl solution in dioxane (11 mL) and stirred at room temperature for 2.5 hours. The solvent was evaporated and the residue was dried giving compound 4 as hydrochloride as white crystals. Yield 1.632 g, 98%. ¹H-NMR (400 MHz, DMSO-D₆) δ_{H}: 1.71 (2H, m), 2.05 (2H, m), 2.98 (2H, m), 3.31 (2H, m), 3.92-4.01 (1H, broad peaks, Z/E forms), 4.95 (2H, superposition of two q, J=7.5 Hz, Z/E forms), 7.31 (1H, superposition of two m, Z/E forms), 7.51-7.68 (1H, broad peaks, Z/E forms), 8.05 (2H, broad peak, Z/E forms), 8.91 (1H, broad peak, Z/E forms), 9.05 (1H, broad peak, Z/E forms), 9.71-9.91 (1H, broad peaks, Z/E forms). MW 420.80. LCMS t_{R} (min): 1.64. MS (APCI+), m/z 421.05, 423.00 [M+H]⁺ (96%).

### Generic Procedure for Synthesis of Final Compounds:

Method A: A mixture of the compound 4 (0.55 mmol), sulfonyl chloride (0.82 mmol), NEt₃ (138 mg, 1.36 mmol), acetonitrile (5 mL) was stirred at room temperature for 8 hours and diluted with water. The formed precipitate was filtered off, purified by recrystallization or/and column chromatography on silica gel giving a final compound.

Method B: A mixture of compound 4 (1.03 mmol), corresponding sulfonyl chloride (1.24 mmol) and K₂CO₃ (3.09 mmol) in dichloromethane (10 mL) was stirred at room temperature for 8 hours, diluted with water and extracted with dichloromethane. The combined organic phases were dried over Na₂SO₄ and concentrated. Purification by column chromatography on silica gel (ethyl acetate/hexane) gave a final compound.

TABLE 37

| En | Structure | IUPAC name | MW | Formula |
|---|---|---|---|---|
| 1 | 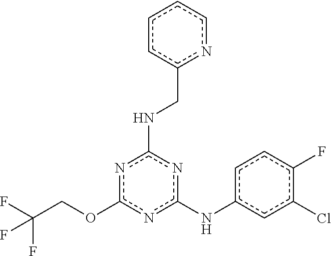 | N-(3-chloro-4-fluorophenyl)-N′- (pyridin-2-ylmethyl)-6-(2,2,2- trifluoroethoxy)-1,3,5-triazine-2,4- diamine | 428.8 | C17H13ClF4N6O |
| 2 | 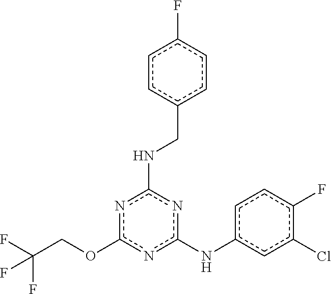 | N-(3-chloro-4-fluorophenyl)-N′-(4- fluorobenzyl)-6-(2,2,2- trifluoroethoxy)-1,3,5-triazine-2,4- diamine | 445.8 | C18H13ClF5N5O |
| 3 | 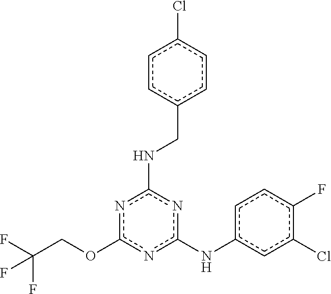 | N-(4-chlorobenzyl)-N′-(3-chloro-4- fluorophenyl)-6-(2,2,2- trifluoroethoxy)-1,3,5-triazine-2,4- diamine | 462.2 | C18H13Cl2F4N5O |
| 4 | 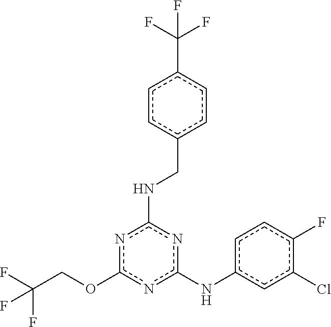 | N-(3-chloro-4-fluorophenyl)-6-(2,2,2- tri-fluoroethoxy)-N′-[4- (trifluoromethyl)benzyl]-1,3,5-triazine- 2,4-diamine | 495.8 | C19H13ClF7N5O |
| 5 | 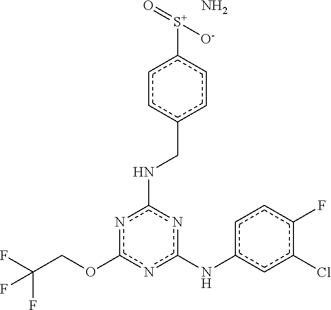 | 4-[({4-[(3-chloro-4- fluorophenyl)amino]-6-(2,2,2- trifluoroethoxy)-1,3,5-triazin-2- yl}amino)methyl]benzenesulfonamide | 506.9 | C18H15ClF4N6O3S |
| 6 | 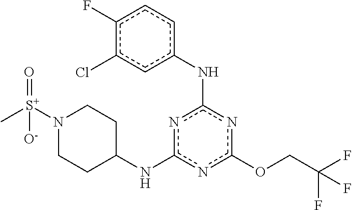 | N-(3-chloro-4-fluorophenyl)-N′-[1- (methylsulfonyl)piperidin-4-yl]-6- (2,2,2-trifluoroethoxy)-1,3,5-triazine- 2,4-diamine | 498.9 | C17H19ClF4N6O3S |
| 7 | 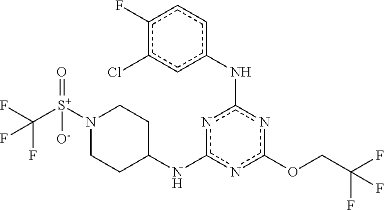 | N-(3-chloro-4-fluorophenyl)-6-(2,2,2- trifluoro-ethoxy)-N′-{1- [(trifluoromethyl)sulfonyl]-piperidin-4- yl}-1,3,5-triazine-2,4-diamine | 552.9 | C17H16ClF7N6O3S |
| 8 | 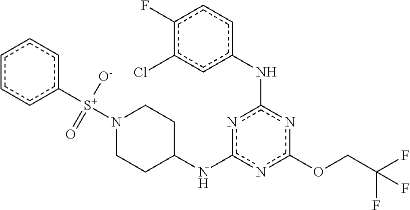 | N-(3-chloro-4-fluorophenyl)-N′-[1- (phenylsulfonyl)piperidin-4-yl]-6- (2,2,2-trifluoroethoxy)-1,3,5-triazine- 2,4-diamine | 561.0 | C22H21ClF4N6O3S |
| 9 | 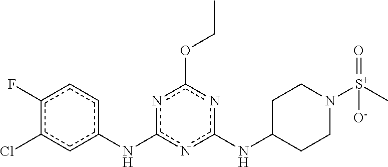 | N-(3-chloro-4-fluorophenyl)-6-ethoxy- N′-[1-(methylsulfonyl)piperidin-4-yl]- 1,3,5-triazine-2,4-diamine | 444.9 | C17H22ClFN6O3S |
| 10 | 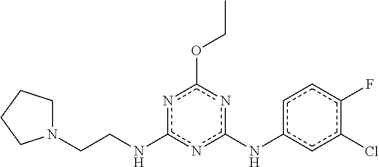 | N-(3-chloro-4-fluorophenyl)-6-ethoxy- N′-[2-pyrrolidin-1-yl)ethyl]-1,3,5- triazine-2,4-diamine | 380.8 | C17H22ClFN6O |
| 11 | 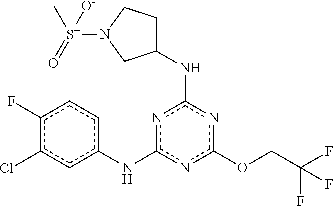 | N-(3-chloro-4-fluorophenyl)-N′-[1- (methylsulfonyl)pyrrolidin-3-yl]-6- (2,2,2-trifluoroethoxy)-1,3,5-triazine- 2,4-diamine | 484.9 | C16H17ClF4N6O3S |
| 12 | 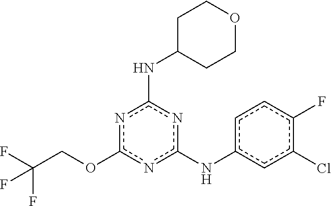 | N-(3-chloro-4-fluorophenyl)-N′- (tetrahydro-2H-pyran-4-yl)-6-(2,2,2- trifluoroethoxy)-1,3,5-triazine-2,4- diamine | 421.8 | C16H16ClF4N5O2 |
| 13 | 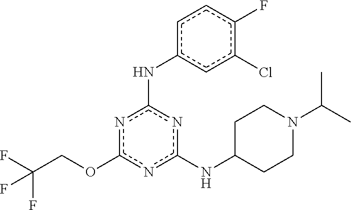 | N-(3-chloro-4-fluorophenyl)-N′-[1- (propan-2-yl)piperidin-4-yl]-6-(2,2,2- trifluoroethoxy)-1,3,5-triazine-2,4- diamine | 462.9 | C19H23ClF4N6O |
| 14 | 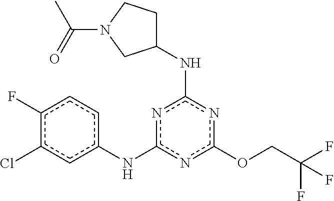 | 1-[3-({4-[(3-chloro-4- fluorophenyl)amino]-6-(2,2,2- trifluoroethoxy)-1,3,5-triazin-2- yl}amino)pyrrolidin-1-yl]ethanone | 448.8 | C17H17ClF4N6O2 |
| 15 | 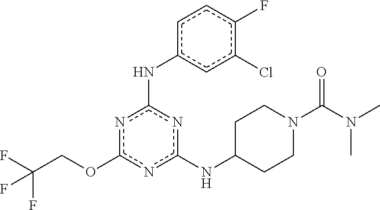 | 4-({4-[(3-chloro-4-fluorophenyl)amino]- 6-(2,2,2-trifluoroethoxy)-1,3,5-triazin-2- yl}-amino)-N,N-dimethylpiperidine-1- carboxamide | 491.9 | C19H22ClF4N7O2 |
| 16 | 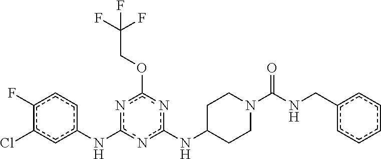 | N-benzyl-4-({4-[(3-chloro-4- fluorophenyl)-amino]-6-(2,2,2- trifluoroethoxy)-1,3,5-triazin-2- yl}amino)piperidine-1-carboxamide | 553.9 | C24H24ClF4N7O2 |
| 17 | 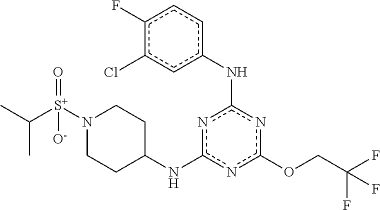 | N-(3-chloro-4-fluorophenyl)-N′-[1- (propan-2-ylsulfonyl)piperidin-4-yl]-6- (2,2,2-trifluoroethoxy)-1,3,5-triazine- 2,4-diamine | 526.9 | C19H23ClF4N6O3S |
| 18 | 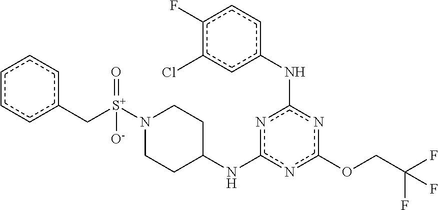 | N-[1-(benzylsulfonyl)piperidin-4-yl]- N′-(3-chloro-4-fluorophenyl)-6-(2,2,2- trifluoroethoxy)-1,3,5-triazine-2,4- diamine | 575.0 | C23H23ClF4N6O3S |
| 19 | 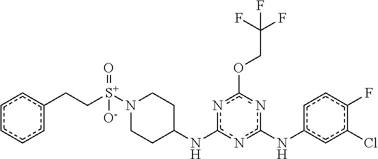 | N-(3-chloro-4-fluorophenyl)-N′-{1-[(2- phenylethyl)sulfonyl]piperidin-4-yl}-6- (2,2,2-trifluoroethoxy)-1,3,5-triazine- 2,4-diamine | 589.0 | C24H25ClF4N6O3S |
| 20 | 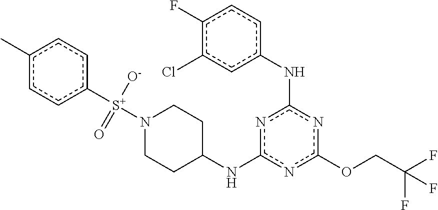 | N-(3-chloro-4-fluorophenyl)-N′-{1-[(4- methyl-phenyl)sulfonyl]piperidin-4- yl}-6-(2,2,2-trifluoroethoxy)-1,3,5- triazine-2,4-diamine | 575.0 | C23H23ClF4N6O3S |
| 21 | 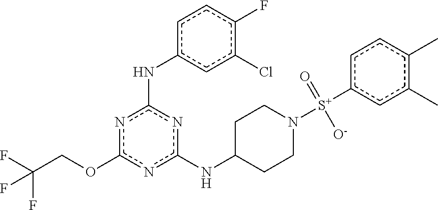 | (N-(3-chloro-4-fluorophenyl)-N′-{1-[(3,4- dimethylphenyl)sulfonyl]piperidin-4- yl}-6-(2,2,2-trifluoroethoxy)-1,3,5- triazine-2,4-diamine | 589.0 | C24H25ClF4N6O3S |
| 22 | 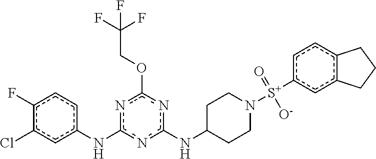 | N-(3-chloro-4-fluorophenyl)-N′-[1- (2,3-dihydro-1H-inden-5- ylsulfonyl)piperidin-4-yl}-6-(2,2,2- trifluoroethoxy)-1,3,5-triazine-2,4- diamine | 601.0 | C25H25ClF4N6O3S |
| 23 | 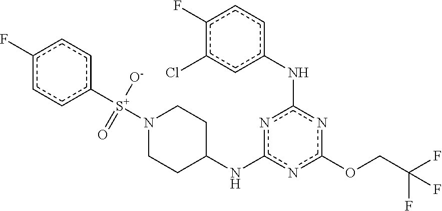 | N-(3-chloro-4-fluorophenyl)-N′-{1-[(4- fluorophenyl)sulfonyl]piperidin-4-yl}- 6-(2,2,2-trifluoroethoxy)-1,3,5- triazine-2,4-diamine | 578.9 | C22H20ClF5N6O3S |
| 24 | 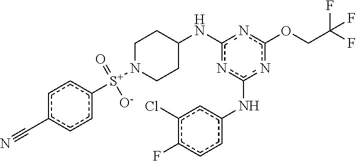 | 4-{[4-({4-[(3-chloro-4- fluorophenyl)amino]-6-(2,2,2- trifluoroethoxy)-1,3,5-triazin-2- yl}amino)piperidin-1- yl]sulfonyl}benzonitrile | 586.0 | C23H20ClF4N7O3S |
| 25 | 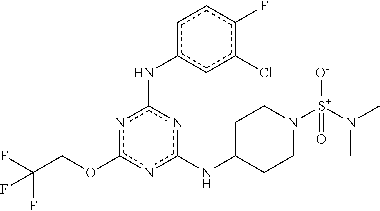 | 4-({4-[(3-chloro-4-fluorophenyl)amino]- 6-(2,2,2-trifluoroethoxy)-1,3,5-triazin-2- yl}-amino)-N,N-dimethylpiperidine-1- sulfonamide | 527.9 | C18H22ClF4N7O3S |
| 26 | 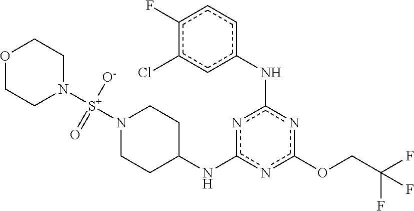 | N-(3-chloro-4-fluorophenyl)-N′-[1- (morpholin-4-ylsulfonyl)piperidin-4-yl]- 6-(2,2,2-trifluoroethoxy)-1,3,5-triazine- 2,4-diamine | 570.0 | C20H24ClF4N7O4S |
| 27 | 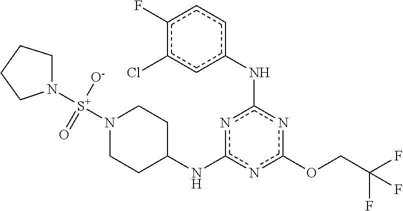 | N-(3-chloro-4-fluorophenyl)-N′-[1- (pyrrolidin-1-ylsulfonyl)piperidin-4-yl]-6- (2,2,2-trifluoroethoxy)-1,3,5-triazine- 2,4-diamine | 554.0 | C20H24ClF4N7O3S |
| 28 | 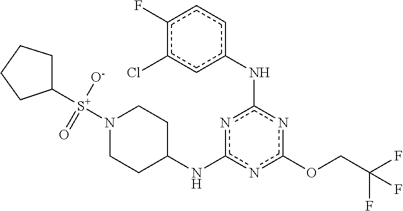 | N-(3-chloro-4-fluorophenyl)-N′-[1- (cyclopentylsulfonyl)piperidin-4-yl]-6- (2,2,2-trifluoroethoxy)-1,3,5-triazine- 2,4-diamine | 553.0 | C21H25ClF4N6O3S |
| 29 | 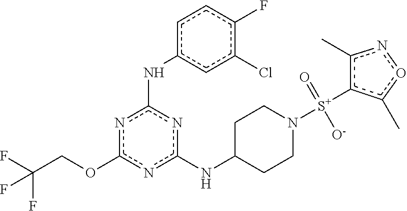 | N-(3-chloro-4-fluorophenyl)-N′-{1-[(3,5- dimethylisoxazol-4- yl)sulfonyl]piperidin-4-yl}-6-(2,2,2- trifluoroethoxy)-1,3,5-triazine-2,4- diamine | 580.0 | C21H22ClF4N7O4S |
| 30 | 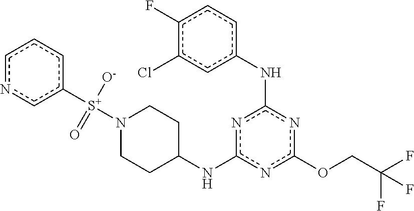 | N-(3-chloro-4-fluorophenyl)-N′-[1- (pyridin-3-ylsulfonyl)piperidin-4-yl]-6- (2,2,2-trifluoroethoxy)-1,3,5-triazine- 2,4-diamine | 561.9 | C21H20ClF4N7O3S |
| 31 | 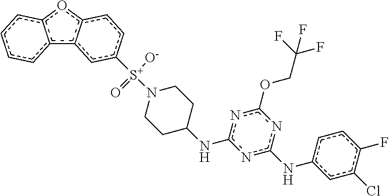 | N-(3-chloro-4-fluorophenyl)-N′-[1- (dibenzo-[b,d]furan-2- ylsulfonyl)piperidin-4-yl]-6-(2,2,2- trifluoroethoxy)-1,3,5-triazine-2,4- diamine | 651.0 | C28H23ClF4N6O4S |
| 32 | 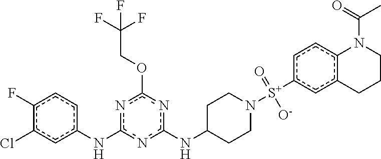 | 1-[6-{[4-({4-[(3-chloro-4- fluorophenyl)amino]-6-(2,2,2- trifluoroethoxy)-1,3,5-triazin-2- yl}amino)piperidin-1-yl]sulfonyl}- 3,4-dihydroquinolin-1(2H)-yl]ethanone | 658.1 | C27H28ClF4N7O4S |
| 33 | 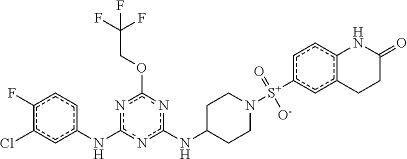 | 6-{[4-({4-[(3-chloro-4- fluorophenyl)amino]-6-(2,2,2- trifluoroethoxy)-1,3,5-triazin-2- yl}amino)piperidin-1-yl]sulfonyl}-3,4- dihydroquinolin-2(1H)-one | 630.0 | C25H24ClF4N7O4S |
| 34 | 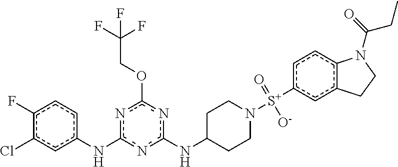 | 1-(5-{[4-({4-[(3-chloro-4- fluorophenyl)amino]-6-(2,2,2- trifluoroethoxy)-1,3,5-triazin-2- yl}amino)piperidin-1-yl]sulfonyl}-2,3- dihydro-1H-indol-1-yl)propan-1-one | 658.1 | C27H28ClF4N7O4S |
| 35 | 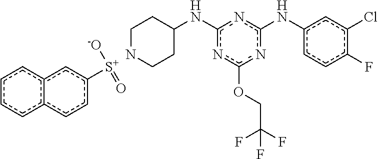 | N-(3-chloro-4-fluorophenyl)-N′-[1- (naphthalen-2-ylsulfonyl)piperidin-4-yl]- 6-(2,2,2-trifluoroethoxy)-1,3,5-triazine- 2,4 diamine | 611.0 | C26H23ClF4N6O3S |
| 36 | 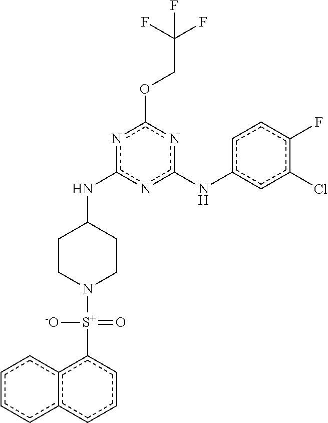 | N-(3-chloro-4-fluorophenyl)-N′-[1- (naphthalen-1-ylsulfonyl)piperidin-4-yl]- 6-(2,2,2-trifluoroethoxy)-1,3,5-triazine- 2,4-diamine | 611.0 | C26H23ClF4N6O3S |
| 37 | 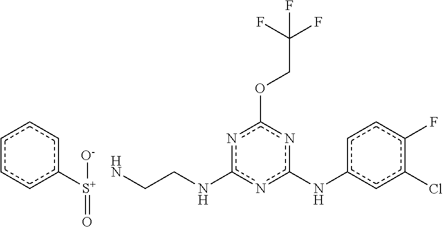 | N-[2-({4-[(3-chloro-4- fluorophenyl)amino]-6-(2,2,2- trifluoroethoxy)-1,3,5-triazin-2- yl}amino)ethyl]benzenesulfonamide | 520.9 | C19H17ClF4N6O3S |
| 38 | 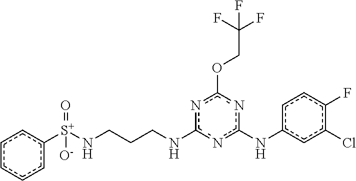 | N-[3-({4-[(3-chloro-4- fluorophenyl)amino]-6-(2,2,2- trifluoroethoxy)-1,3,5-triazin-2- yl}amino)propyl]benzenesulfonamide | 534.9 | C20H19ClF4N6O3S |
| 39 | 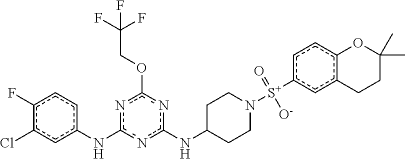 | N-(3-chloro-4-fluorophenyl)-N′-{1-[(2,2- dimethyl-3,4-dihydro-2H-chromen-6- yl)sulfonyl]piperidin-4-yl}-6-(2,2,2- trifluoroethoxy)-1,3,5-triazine-2,4- diamine | 645.1 | C27H29ClF4N6O4S |
| 40 | 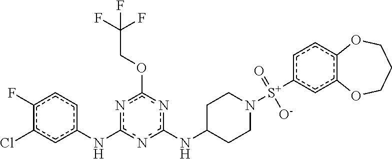 | N-(3-chloro-4-fluorophenyl)-N′-[1-(3,4- dihydro-2H-1,5-benzodioxepin-7- ylsulfonyl)piperidin-4-yl]-6-(2,2,2- trifluoroethoxy)-1,3,5-triazine-2,4- diamine | 633.0 | C25H25ClF4N6O5S |

### Procedures and Analytical Data for Table 37.

### 1. N-(3-Chloro-4-fluoro-phenyl)-N′-pyridin-2-ylmethyl-6-(2,2,2-trifluoro-ethoxy)-[1,3,5]triazine-2,4-diamine

¹H-NMR (400 MHz, DMSO-D₆) δ_{H}: 4.62 (2H, broad), 4.82-5.00 (2H, two q, J=7.5 Hz, Z/E forms), 7.22 (1H, broad), 7.23-7.30 (1H, broad, Z/E forms), 7.32 (1H, broad), 7.45-7.68 (1H, broad, Z/E forms), 7.73 (1H, t, J=8.0 Hz), 7.83-8.07 (1H, broad, Z/E forms), 8.28 (1H, broad), 8.51 (1H, broad t, J=7.5 Hz), 9.75 (1H, broad). MW 428.78. LCMS t_{R} (min): 1.71. MS (APCI), m/z 429.02, 431.00 [M+H]⁺. HPLC t_{R} (min): 11.15. M_{P} 159-161° C.

### 2. N-(3-Chloro-4-fluoro-phenyl)-N′-(4-fluoro-benzyl)-6-(2,2,2-trifluoro-ethoxy)-[1,3,5]triazine-2,4-diamine

¹H-NMR (400 MHz, DMSO-D₆) δ_{H}: 4.50 (2H, broad), 4.95 (2H, superposition of two q, J=7.5 Hz, Z/E forms), 7.15 (2H, m, J=8.5 Hz), 7.31-7.35 (1H, broad, Z/E forms), 7.37 (2H, broad), 7.53-7.65 (1H, broad, Z/E forms), 7.95-8.02 (1H, two broad d, J=8.5 Hz, Z/E forms), 8.30 (1H, broad), 9.70-9.82 (1H, broad, Z/E forms).

MW 445.78. LCMS t_{R} (min): 2.10. MS (APCI), m/z 446.11, 448.10 [M+H]⁺. HPLC t_{R} (min): 16.81. M_{P} 174-176° C.

### 3. N-(3-Chloro-4-fluoro-phenyl)-6-(2,2,2-trifluoro-ethoxy)-N′-(4-trifluoromethyl-benzyl)-[1,3,5]triazine-2,4-diamine

¹H-NMR (400 MHz, DMSO-D₆) δ_{H}: 4.61 (2H, broad), 4.90 (1H, superposition of two q, J=7.5 Hz, Z/E forms), 7.27 (1H, superposition of two m, J=8.5 Hz, Z/E forms), 7.45-7.53 (1H, two broad signals, Z/E forms), 7.52 (2H, broad doublet, J=8.5 Hz, Z/E forms), 7.63 (1H, broad), 7.67 (2H, broad d, J=8.5 Hz, Z/E forms), 7.85-8.02 (1H, two broad signals, Z/E forms), 8.36 (1H, broad), 9.72-9.80 (1H, two broad signals, Z/E forms). MW 495.79. LCMS t_{R} (min): 2.17. MS (APCI), m/z 496.14, 498.15 [M+H]⁺. HPLC t_{R} (min): 17.60. M_{P} 185-187° C.

### 4. N-(4-Chloro-benzyl)-N′-(3-chloro-4-fluoro-phenyl)-6-(2,2,2-trifluoro-ethoxy)-[1,3,5]triazine-2,4-diamine

¹H-NMR (400 MHz, DMSO-D₆) δ_{H}: 4.50 (2H, broad, Z/E forms), 4.95 (2H, superposition of two q, J=7.5 Hz, Z/E forms), 7.35 (5H, broad), 7.50-7.63 (1H, two broad signals, Z/E forms), 7.91-8.03 (1H, two broad signals, Z/E forms), 8.30 (1H, broad), 9.71-9.80 (1H, two broad signals, Z/E forms). MW 462.24. LCMS t_{R} (min): 2.17. MS (APCI), m/z 462.03, 464.03 [M+H]⁺. HPLC t_{R} (min): 18.03. M_{P} 199-201° C.

### 5. 4-{[4-(3-Chloro-4-fluoro-phenylamino)-6-(2,2,2-trifluoro-ethoxy)-[1,3,5]triazin-2-ylamino]-methyl}-benzenesulfonamide

¹H-NMR (400 MHz, DMSO-D₆) δ_{H}: 4.58 (2H, broad), 4.92 (2H, superposition of two q, J=7.5 Hz, Z/E forms), 7.25 (2H, broad m), 7.40 (2H, broad), 7.47 (2H, d, J=8.5 Hz), 7.62-7.89 (1H, two broad signals, Z/E forms), 7.76 (2H, d, J=8.5 Hz), 8.01-8.34 (1H, two broad signals, Z/E forms), 9.70 (1H, broad). MW 506.87. LCMS t_{R} (min): 1.85. MS (APCI), m/z 507.17, 509.17 [M+H]⁺. HPLC t_{R} (min): 14.04. M_{P} 206-208° C.

### 6. N-(3-Chloro-4-fluoro-phenyl)-N′-(1-methanesulfonyl-piperidin-4-yl)-6-(2,2,2-trifluoro-ethoxy)-[1,3,5]triazine-2,4-diamine

¹H-NMR (400 MHz, DMSO-D₆) δ_{H}: 1.64 (2H, broad), 1.95 (2H, m), 2.85 (5H, broad), 3.60 (2H, broad), 3.90 (1H, broad), 4.95 (2H, broad q, J=7.5 Hz), 7.32 (1H, superposition of two t, J=8.5 Hz, Z/E forms), 7.55-7.69 (1H, two broad signals, Z/E forms), 7.75-7.85 (1H, two broad signals, Z/E forms), 7.96-8.08 (1H, two broad signals, Z/E forms), 9.65-9.82 (1H, two broad signals, Z/E forms). MW 498.89. LCMS t_{R} (min): 1.92. MS (APCI), m/z 499.08, 501.08 [M+H]⁺. HPLC t_{R} (min): 14.72. M_{P} 250-252° C.

### 7. N-(3-Chloro-4-fluoro-phenyl)-6-(2,2,2-trifluoro-ethoxy)-N′-(1-trifluoromethanesulfonyl-piperidin-4-yl)-[1,3,5]triazine-2,4-diamine

¹H-NMR (400 MHz, DMSO-D₆) δ_{H}: 1.60 (2H, broad, Z/E forms), 2.01 (2H, broad, Z/E forms), 3.35 (2H, broad m), 3.83 (2H, broad, Z/E forms), 4.00-4.18 (1H, two broad signals, Z/E forms), 4.95 (2H, broad q, J=7.5 Hz, Z/E forms), 7.32 (1H, superposition of two m, Z/E forms), 7.55-7.67 (1H, two broad signals, Z/E forms), 7.75-7.87 (1H, two broad signals, Z/E forms), 7.98-8.05 (1H, two broad signals, Z/E forms), 9.61-9.82 (1H, two broad signals, Z/E forms). MW 552.86. LCMS t_{R} (min): 2.14. MS (APCI), m/z 553.04, 555.22 [M+H]⁺. HPLC t_{R} (min): 17.24. M_{P} 158-160° C.

### 8. N-(1-Benzenesulfonyl-piperidin-4-yl)-N′-(3-chloro-4-fluoro-phenyl)-6-(2,2,2-trifluoro-ethoxy)-[1,3,5]-triazine-2,4-diamine (730

¹H-NMR (400 MHz, DMSO-D₆) δ_{H}: 1.58 (2H, m), 1.91 (2H, m), 2.41 (2H, m), 3.65 (2H, broad, Z/E forms), 3.65-3.81 (1H, broad, Z/E forms), 4.40 (2H, superposition of two broad q, J-7.5 Hz, Z/E forms), 7.23-7.37 (2H, m), 7.66 (3H, m), 7.74 (2H, d, J=8.5 Hz), 7.74-7.82 (1H, two broad signals, Z/E forms), 7.95-8.15 (1H, two broad signals, Z/E forms), 9.60-9.80 (1H, two broad signals, Z/E forms). MW 560.96. LCMS t_{R} (min): 2.09. MS (APCI), m/z 561.09, 563.11 [M+H]⁺. HPLC t_{R} (min): 16.38. M_{P} 205-208° C.

### 9. N-(3-Chloro-4-fluoro-phenyl)-6-ethoxy-N′-(1-methanesulfonyl-piperidin-4-yl)-[1,3,5]triazine-2,4-diamine

To a solution of ethanol (69.7 mg, 1.52 mmol) in THF (3 mL) sodium hydride (60.6 mg, 2.53 mmol) was added. The obtained mixture was stirred at room temperature for 10 minutes. Then the mixture was added to a solution of 6-Chloro-N-(3-chloro-4-fluoro-phenyl)-N-(1-methanesulfonyl-piperidin-4-yl)-[1,3,5]triazine-2,4-diamine (220 mg, 0.51 mmol) in THF (7 mL). The resulting mixture was stirred at refluxing for 2 hours and diluted with water. The formed solid was collected by filtration. Purification by column chromatography (silica gel, hexane/ethyl acetate, 1/2) and recrystallization from hexane gave a final compound. Yield 163 mg, 73%. ¹H-NMR (400 MHz, DMSO-D₆) δ_{H}: 1.28 (3H, broad), 1.58 (2H, broad), 1.95 (2H, broad), 2.85 (5H, superposition of s and m), 3.59 (2H, broad), 3.90 (1H, broad), 4.30 (2H, broad q, J=7.5 Hz), 7.31 (1H, broad), 7.43-7.57 (1H, two broad peaks, Z/E forms), 7.57-7.70 (1H, two broad peaks, Z/E forms), 8.00-8.10 (1H, two broad peaks), 9.40-9.58 (1H, two broad peaks). MW 444.92. LCMS t_{R} (min): 1.82. MS (APCI), m/z 445.11 [M+H]⁺. HPLC t_{R} (min): 13.18. M_{P} 197-199° C.

### 10. N-(3-Chloro-4-fluoro-phenyl)-6-ethoxy-N′-(2-pyrrolidin-1-yl-ethyl)-[1,3,5]triazine-2,4-diamine

A solution of (3-Chloro-4-fluoro-phenyl)-(4,6-dichloro-[1,3,5]triazin-2-yl) (152 mg, 0.50 mmol), 2-pyrrolidin-1-yl-ethylamine (63 mg, 0.55 mmol) and NEt₃ (101 mg, 1.00 mmol) in MeCN (7 mL) was refluxed for 1.5 hour (TLC control). Then, the reaction mixture was cooled down to room temperature, concentrated and extracted with dichloromethane. The combined organic phases were washed with water, dried over Na₂SO₄, and concentrated. Purification by column chromatography on silica gel (EtOAc/MeOH) gave a final compound as a white crystalline solid. Yield 120 mg, 63%. ¹H-NMR (400 MHz, DMSO-D₆) δ_{H}: 1.30 (3H, broad), 1.70 (4H, broad), 2.59 (4H, broad peak), 2.70 (2H, broad peak), 3.42 (2H, broad peak), 4.30 (2H, broad peak), 7.27 (1H, d/d, J=8.5/8.0 Hz), 7.32 (1H, broad peak, Z/E forms), 7.62 (1H, broad peak, Z/E forms), 8.05 (1H, broad peak, Z/E forms), 9.42-9.52 (1H, two broad peaks, Z/E forms). MW 380.85. LCMS t_{R} (min): 1.55. MS (APCI+), m/z 381.10, 383.09 [M+H]⁺. HPLC t_{R} (min): 10.29. M_{P} 111-113° C.

### 11. N-(3-Chloro-4-fluoro-phenyl)-N′-(1-methanesulfonyl-pyrrolidin-3-yl)-6-(2,2,2-trifluoro-ethoxy)-[1,3,5]triazine-2,4-diamine

To a solution of (3-Chloro-4-fluoro-phenyl)-(4,6-dichloro-[1,3,5]triazin-2-yl)-amine (300 mg, 1.02 mmol) and 1-methanesulfonyl-pyrrolidin-3-ylamine (201 mg, 1.02 mmol) in acetonitrile (3 mL) DIPEA (264 mg, 2.04 mmol) was added. The reaction mixture was stirred at room temperature for 16 hours, diluted with water and extracted with chloroform. The combined organic phases were dried over sodium sulfate and concentrated. Purification by column chromatography on silica gel (20% methanol/chloroform) gave 6-Chloro-N-(3-chloro-4-fluoro-phenyl)-N′-(1-methanesulfonyl-pyrrolidin-3-yl)-[1,3,5]triazine-2,4-diamine. Yield 420 mg, 98%.

A mixture of 6-Chloro-N-(3-chloro-4-fluoro-phenyl)-N′-(1-methanesulfonyl-pyrrolidin-3-yl)-[1,3,5]-triazine-2,4-diamine (210 mg, 0.5 mmol), 2,2,2-trifluoro-ethanol (2 mL) and K₂CO₃ (138 mg, 1.0 mmol) was stirred at refluxing for 4 hours, cooled down to room temperature and diluted with water. The formed solid was collected by filtration, washed with water and dried giving a final compound. Yield 176 mg, 73%. ¹H-NMR (400 MHz, DMSO-D₆) δ_{H}: 1.97 (1H, m), 2.21 (1H, m), 2.90 (3H, s), 3.20 (1H, m) 3.30 (1H, m), 3.42 (1H, m), 3.58 (1H, m), 4.41-4.50 (1H, two broad peaks, Z/E forms), 4.93 (2H, superposition of two q, J=7.5 Hz, Z/E forms), 7.31 (1H, superposition of two m, Z/E forms), 7.58-7.66 (1H, two broad peaks, Z/E forms), 7.99 (2H, broad), 9.75-9.85 (1H, two broad peaks, Z/E forms). MW 484.86. LCMS t_{R} (min): 1.96. MS (APCI), m/z 485.36 [M+H]⁺. HPLC t_{R} (min): 14.78. M_{P} 199-200° C.

### 12. N-(3-Chloro-4-fluoro-phenyl)-N′-(tetrahydro-pyran-4-yl)-6-(2,2,2-trifluoro-ethoxy)-[1,3,5]triazine-2,4-diamine

To a suspension of tetrahydro-pyran-4-ylamine (826 mg, 6.0 mmol) and DIPEA (1.55 g, 12.0 mmol) in THF (15 mL) (3-Chloro-4-fluoro-phenyl)-(4,6-dichloro-[1,3,5]triazin-2-yl)-amine (1.76 g, 6.0 mmol) was added portionwise at room temperature. The resulting mixture was stirred at room temperature for 8 hours, diluted with water and extracted with THF. The combined organic phases were dried over Na₂SO₄, concentrated at reduced pressure and dried giving a final compound. Yield 1.767 g, 82%.

A mixture of 6-Chloro-N-(3-chloro-4-fluoro-phenyl)-N′-(tetrahydro-pyran-4-yl)-[1,3,5]triazine-2,4-diamine (358 mg, 1.0 mmol), 2,2,2-trifluoro-ethanol (300 mg, 3.0 mmol), K₂CO₃ (333 mg, 2.4 mmol) and DMSO (5.0 mL) was stirred at 100° C. for 4.5 hours (TLC control), cooled down to room temperature and diluted with water. The formed solid was collected by filtration and washed with water. Purification by recrystallization from acetonitrile gave a final compound. Yield 200 mg, 48%. ¹H-NMR (400 MHz, DMSO-D₆) δ_{H}: 1.55 (2H, m), 1.82 (2H, m), 3.39 (2H, m), 3.88 (2H, broad), 4.00 (1H, broad), 4.95 (2H, broad q, J=7.5 Hz), 7.32 (1H, superposition of two m, Z/E forms), 7.50-7.68 (1H, two broad peaks, Z/E forms), 7.75-7.97 (1H, two broad peaks, Z/E forms), 7.97-8.14 (1H, two broad peaks), 9.57-9.78 (1H, two broad peaks). MW 421.78. LCMS t_{R} (min): 1.96. MS (APCI), m/z 422.05 [M+H]⁺. HPLC t_{R} (min): 15.44. M_{P} 190-191° C.

### 13. N-(3-Chloro-4-fluoro-phenyl)-N′-(1-isopropyl-piperidin-4-yl)-6-(2,2,2-trifluoro-ethoxy)-[1,3,5]triazine-2,4-diamine

¹H-NMR (400 MHz, DMSO-D₆) δ_{H}: 0.98 (6H, d, J=7.5 Hz), 1.50 (2H, m), 1.84 (2H, m), 2.21 (2H, m), 2.70 (1H, broad), 2.80 (2H, broad), 3.72 (1H, broad, Z/E forms), 4.92 (2H, broad q, J=7.5 Hz), 7.31 (1H, superposition of two t, J=8.0 Hz, Z/E forms), 7.47-7.62 (1H, two broad peaks, Z/E forms), 7.68 (1H, broad, Z/E forms), 7.95-8.20 (1H, two broad peaks, Z/E forms), 9.55-9.78 (1H, two broad peaks, Z/E forms).

MW 462.88. LCMS t_{R} (min): 1.67. MS (APCI), m/z 463.11; 465.09 [M+H]⁺. HPLC t_{R} (min): 11.70. M_{P} 100-102° C.

### 14. 1-{3-[4-(3-Chloro-4-fluoro-phenylamino)-6-(2,2,2-trifluoro-ethoxy)-[1,3,5]-triazin-2-ylamino]-pyrrolidin-1-yl}-ethanone

¹H-NMR (400 MHz, DMSO-D₆) δ_{H}: 1.91 (4H, superposition of s and m), 2.09-2.21 (1H, two broad peaks, Z/E forms), 3.32 (1H, broad), 3.45 (1H, broad), 3.59 (1H, broad), 3.61-3.78 (1H, two broad peaks), 4.45 (1H, broad, Z/E forms), 4.95 (2H, broad, Z/E forms), 7.30 (1H, broad, Z/E forms), 7.57-7.70 (1H, two broad peaks, Z/E forms), 7.99 (1H, broad, Z/E forms), 8.02-8.26 (1H, two broad peaks), 9.60-9.80 (1H, two broad peaks, Z/E forms). MW 448.81. LCMS t_{R} (min): 1.78. MS (APCI), m/z 449.09 [M+H]⁺. HPLC t_{R} (min): 13.68. M_{P} 106-108° C.

### 14c15. 4-[4-(3-Chloro-4-fluoro-phenylamino)-6-(2,2,2-trifluoro-ethoxy)-[1,3,5]triazin-2-ylamino]-piperidine-1-carboxylic acid dimethylamide

¹H-NMR (400 MHz, DMSO-D₆) δ_{H}: 1.48 (2H, m), 1.84 (2H, m), 2.72 (6H, s), 2.80 (2H, broad), 3.58 (2H, broad, Z/E forms), 3.91 (1H, broad, Z/E forms), 4.93 (2H, broad q, J=7.5 Hz), 7.31 (1H, superposition of two t, J=8.5/8.0 Hz, Z/E forms), 7.51-7.66 (1H, two broad peaks, Z/E forms), 7.66-7.75 (1H, two broad peaks, Z/E forms), 7.98-8.12 (1H, two broad peaks, Z/E forms), 9.60-9.80 (1H, two broad peaks, Z/E forms). MW 491.87. LCMS t_{R} (min): 1.90. MS (APCI), m/z 492.03; 494.00 [M+H]⁺. HPLC t_{R} (min): 14.93. M_{P} 152-153° C.

### 16. 4-[4-(3-Chloro-4-fluoro-phenylamino)-6-(2,2,2-trifluoro-ethoxy)-[1,3,5]triazin-2-ylamino]-piperidine-1-carboxylic acid benzylamide

¹H-NMR (400 MHz, DMSO-D₆) δ_{H}: 1.42 (2H, m), 1.84 (2H, m), 2.82 (2H, m), 4.00 (3H, broad), 4.23 (2H, d, J=7.5 Hz, Z/E forms), 4.93 (2H, superposition of two q, J=7.5 Hz, Z/E forms), 7.00 (1H, broad), 7.26 (6H, broad), 7.52-7.66 (1H, broad peak, Z/E forms), 7.68-7.77 (1H, broad peak, Z/E forms), 7.98-8.13 (1H, broad peak, Z/E forms), 9.60-9.80 (1H, broad peak, Z/E forms). MW 553.95. LCMS t_{R} (min): 2.00. MS (APCI), m/z 554.05; 556.05 [M+H]⁺. HPLC t_{R} (min): 15.71. M_{P} 190-192° C.

### 17. N-(3-Chloro-4-fluoro-phenyl)-N′-[1-(propane-2-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.22 (6H, d, J=7.5 Hz), 1.52 (2H, m), 1.91 (2H, m), 3.02 (2H, m), 3.32 (1H, m), 3.68 (2H, broad, Z/E forms), 3.95 (1H, broad, Z/E forms), 4.95 (2H, broad q. J=7.5 Hz, Z/E forms), 7.32 (1H, superposition of two t, J=8.5/8.0 Hz, Z/E forms), 7.51-7.68 (1H, two broad peaks, Z/E forms), 7.68-7.81 (1H, two broad peaks, Z/E forms), 7.98-8.10 (1H, two broad peaks, Z/E forms), 9.60-9.80 (1H, two broad peaks, Z/E forms). MW 526.94. LCMS t_{R} (min): 2.02. MS (APCI), m/z 527.06; 529.05 [M+H]⁺. HPLC t_{R} (min): 16.05. M_{P} 218-220° C.

### 18. N-(3-Chloro-4-fluoro-phenyl)-N′-(1-phenylmethanesulfonyl-piperidin-4-yl)-6-(2,2,2-trifluoro-ethoxy)-[1,3,5]triazine-2,4-diamine

¹H-NMR (400 MHz, DMSO-D₆) δ_{H}: 1.50 (2H, m), 1.89 (2H, m), 2.82 (2H, m), 3.59 (2H, broad), 3.88 (1H, broad), 4.40 (2H, superposition of two s, Z/E forms), 4.95 (2H, broad q, J=7.5 Hz), 7.30-7.53 (1H, two broad peaks, Z/E forms), 7.38 (6H, m), 7.68-7.78 (1H, two broad peaks, Z/E forms), 7.97-8.08 (1H, two broad peaks, Z/E forms), 9.60-9.80 (1H, two broad peaks, Z/E forms). MW 574.98. LCMS t_{R} (min): 2.10. MS (APCI), m/z 575.25 [M+H]⁺. HPLC t_{R} (min): 16.77. M_{P} 214-216° C.

### 19. N-(3-Chloro-4-fluoro-phenyl)-N′-[1-(2-phenyl-ethanesulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.55 (2H, broad), 1.93 (2H, broad), 2.99 (4H, m), 3.35 (2H, broad), 3.65 (2H, broad), 3.92 (1H, broad), 4.92 (2H, broad q, J=7.5 Hz), 7.24 (1H, broad), 7.31 (5H, broad), 7.55-7.68 (1H, two broad peaks, Z/E forms), 7.73-7.81 (1H, two broad peaks, Z/E forms), 7.98-8.08 (1H, two broad peaks, Z/E forms), 9.60-9.80 (1H, two broad peaks, Z/E forms). MW 589.01. LCMS t_{R} (min): 2.14. MS (APCI), m/z 589.09 [M+H]⁺. HPLC t_{R} (min): 17.15. M_{P} 155-157° C.

### 20. N-(3-Chloro-4-fluoro-phenyl)-N′-[1-(toluene-4-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.59 (2H, broad), 1.90 (2H, broad peaks), 2.35 (3H, s), 2.40 (2H, broad peaks), 3.60 (2H, broad peaks), 3.72 (1H, broad peak), 4.90 (2H, broad q, J=7.5 Hz), 7.24 (1H, broad m), 7.27-7.37 (1H, broad peak, Z/E forms), 7.44 (2H, d, J=8.5 Hz), 7.62 (2H, d, J=8.5 Hz), 7.65-7.81 (1H, broad peak, Z/E forms), 7.93-8.12 (1H, broad peak, Z/E forms), 9.55-9.78 (1H, broad peak, Z/E forms). MW 574.98. LCMS t_{R} (min): 2.15. MS (APCI), m/z 575.06; 577.07 [M+H]⁺. HPLC t_{R} (min): 17.51. M_{P} 228-229° C.

### 21. N-(3-Chloro-4-fluoro-phenyl)-N′-[1-(3,4-dimethyl-benzenesulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.53 (2H, m), 1.90 (2H, m), 2.75 (6H, s), 2.97 (2H, m), 3.60 (2H, m), 3.92 (1H, broad, Z/E forms), 4.95 (2H, broad, Z/E forms), 7.32 (1H, broad m, Z/E forms), 7.52-7.69 (1H, two broad peaks, Z/E forms), 7.69-7.80 (1H, two broad peaks, Z/E forms), 7.98-8.12 (1H, two broad peaks, Z/E forms), 9.60-9.80 (1H, two broad peaks, Z/E forms). MW 589.02. LCMS t_{R} (min): 2.22. MS (APCI), m/z 589.02 [M+H]⁺. HPLC t_{R} (min): 17.74. M_{P} 250-253° C.

### 22. N-(3-Chloro-4-fluoro-phenyl)-N′-[1-(indane-5-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.67 (2H, broad), 1.91 (2H, broad), 2.08 (2H, broad), 2.36 (2H, m), 2.95 (4H, broad), 3.58 (2H, broad), 3.77 (1H, broad), 4.90 (2H, broad q, J=7.5 Hz), 7.25 (2H, broad m), 7.47 (2H, broad peak, Z/E forms), 7.56 (1H, broad peak, Z/E forms), 7.65-7.82 (1H, broad peak, Z/E forms), 7.93-8.14 (1H, broad peak, Z/E forms), 9.55-9.78 (1H, broad peak, Z/E forms). MW 601.00. LCMS t_{R} (min): 2.25. MS (APCI), m/z 601.11; 603.09 [M+H]⁺. HPLC t_{R} (min): 18.12.

### 23. N-(3-Chloro-4-fluoro-phenyl)-N′-[1-(4-fluoro-benzenesulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.61 (2H, m), 1.95-2.08 (2H, broad, Z/E forms), 2.84 (3H, s), 2.98 (2H, m), 3.60 (2H, broad), 4.00 (1H, broad), 5.00 (2H, broad q, J=7.5 Hz), 7.92 (1H, broad peak, Z/E forms), 8.00 (1H, superposition of two d, J=8.5 Hz, Z/E forms), 8.07-8.14 (1H, two d, J=8.5 Hz, Z/E forms), 8.67 (1H, d, J=7.5 Hz, Z/E forms), 8.76 (1H, s), 8.83 (1H, s), 10.00-10.18 (1H, broad peak, Z/E forms). MW 578.95. LCMS t_{R} (min): 2.11. MS (APCI), m/z 579.05; 581.02 [M+H]⁺. HPLC t_{R} (min): 16.94. M_{P} 224-226° C.

### 24. 4-{4-[4-(3-Chloro-4-fluoro-phenylamino)-6-(2,2,2-trifluoro-ethoxy)-[1,3,5]-triazin-2-ylamino]-piperidine-1-sulfonyl}-benzonitrile

¹H-NMR (400 MHz, DMSO-D₆) δ_{H}: 1.55 (2H, broad peak), 1.92 (2H, broad peak), 2.59 (2H, broad peak), 3.67 (2H, broad peak), 3.79 (1H, broad peak), 4.90 (2H, broad q, J=7.5 Hz), 7.28 (1H, broad peak), 7.41-7.65 (1H, broad peak, Z/E forms), 7.65-7.80 (1H, broad peak, Z/E forms), 7.98 (2H, broad peak, Z/E forms), 8.12 (3H, broad peak, Z/E forms), 9.58-9.77 (1H, broad peaks, Z/E forms). MW 585.97. LCMS t_{R} (min): 2.08. MS (APCI), m/z 586.08 [M+H]⁺. HPLC t_{R} (min): 16.54. M_{P} 247-24800.

### 25. 4-[4-(3-Chloro-4-fluoro-phenylamino)-6-(2,2,2-trifluoro-ethoxy)-[1,3,5]triazin-2-ylamino]-piperidine-1-sulfonic acid dimethylamide

¹H-NMR (400 MHz, DMSO-D₆) δ_{H}: 1.53 (2H, m), 1.90 (2H, m), 2.75 (6H, s), 2.97 (2H, m), 3.60 (2H, m), 3.92 (1H, broad, Z/E forms), 4.95 (2H, broad, Z/E forms), 7.32 (1H, broad m, Z/E forms), 7.52-7.69 (1H, two broad peaks, Z/E forms), 7.69-7.80 (1H, two broad peaks, Z/E forms), 7.98-8.12 (1H, two broad peaks, Z/E forms), 9.60-9.80 (1H, two broad peaks, Z/E forms). MW 527.93. LCMS t_{R} (min): 1.98. MS (APCI), m/z 528.08; 530.06 [M+H]⁺. HPLC t_{R} (min): 16.05.

### 26. N-(3-Chloro-4-fluoro-phenyl)-N′-[1-(morpholine-4-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.55 (2H, m), 1.92 (2H, m), 3.12 (2H, broad peak, Z/E forms), 3.20 (4H, broad peak), 3.62 (6H, broad peak, Z/E forms), 3.95 (1H, broad peak, Z/E forms), 4.95 (2H, broad q, J=7.5 Hz), 7.32 (1H, superposition of two m, Z/E forms), 7.52-7.67 (1H, two broad peak, Z/E forms), 7.72-7.82 (1H, two broad peak, Z/E forms), 7.98-8.11 (1H, two broad peak, Z/E forms), 9.60-9.81 (1H, two broad peak, Z/E forms). MW 569.96. LCMS t_{R} (min): 1.96. MS (APCI+), m/z 570.09, 572.09 [M+H]⁺. HPLC t_{R} (min): 16.08. M_{P} 172-174° C.

### 27. N-(3-Chloro-4-fluoro-phenyl)-N′-[1-(pyrrolidine-1-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.57 (2H, m), 1.85 (4H, m), 1.94 (2H, m), 2.92 (2H, t, J=7.5 Hz), 3.20 (4H, m), 3.60 (2H, broad peak, Z/E forms), 3.90 (1H, broad peak, Z/E forms), 4.95 (2H, broad q, J=7.5 Hz), 7.34 (1H, superposition of two m), 7.52-7.68 (1H, two broad peaks, Z/E forms), 7.68-7.82 (1H, two broad peaks, Z/E forms), 8.00-8.18 (1H, two broad peaks, Z/E forms), 9.62-9.82 (1H, two broad peaks, Z/E forms). MW 553.96. LCMS t_{R} (min): 1.53. MS (APCI+), m/z 554.09, 556.10 [M+H]⁺. HPLC t_{R} (min): 17.15. M_{P} 185-187° C.

### 28. N-(3-Chloro-4-fluoro-phenyl)-N′-(1-cyclopentanesulfonyl-piperidin-4-O-6-(2,2,2-trifluoro-ethoxy)-[1,3,5]triazine-2,4-diamine

¹H-NMR (400 MHz, DMSO-D₆) δ_{H}: 1.61 (4H, m), 1.65 (2H, m), 1.81 (2H, m), 2.05 (4H, m), 3.01 (2H, m), 3.61 (3H, m), 3.95 (1H, broad peak, Z/E forms), 4.95 (2H, broad q, J=7.5 Hz), 7.31 (1H, superposition of m, Z/E forms), 7.51-7.69 (1H, two broad peaks, Z/E forms), 7.69-7.81 (1H, two broad peaks, Z/E forms), 8.01-8.15 (1H, two broad peaks, Z/E forms), 9.61-9.81 (1H, two broad peaks, Z/E forms). MW 552.98. LCMS t_{R} (min): 2.22. MS (APCI+), m/z 553.08, 555.06 [M+H]⁺. HPLC t_{R} (min): 17.41. M_{P} 216-218° C.

### 29. N-(3-Chloro-4-fluoro-phenyl)-N-′-[1-(3,5-dimethyl-isoxazole-4-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.70 (2H, m), 1.98 (2H, m), 2.35 (3H, s), 2.62 (3H, s), 2.78 (2H, m), 3.60 (2H, m), 3.90 (1H, broad peak, Z/E forms), 4.95 (2H, q, J=7.5 Hz), 7.30 (1H, superposition of two m, Z/E forms), 7.45-7.67 (1H, two broad peaks, Z/E forms), 7.72-7.85 (1H, two broad peaks, Z/E forms), 7.96-8.13 (1H, two broad peaks, Z/E forms), 9.62-9.83 (1H, two broad peaks, Z/E forms). MW 579.96. LCMS t_{R} (min): 2.08. MS (APCI+), m/z 580.05, 582.04 [M+H]⁺. HPLC t_{R} (min): 17.43. M_{P} 232-234° C.

### 30. N-(3-Chloro-4-fluoro-phenyl)-N′-[1-(pyridine-3-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.61 (2H, m), 1.97 (2H, m), 2.61 (2H, m), 3.71 (2H, m), 3.72-3.85 (1H, two broad peaks, Z/E forms), 4.91 (2H, broad q, J=7.5 Hz), 7.25 (1H, superposition of two m), 7.25-7.38 (1H, two broad peaks, Z/E forms), 7.68 (1H, broad peak, Z/E forms), 7.68-7.79 (1H, two broad peaks, Z/E forms), 7.91-8.09 (1H, two broad peaks, Z/E forms), 8.18 (1H, broad peak, Z/E forms), 8.92 (2H, superposition of two broad peaks, Z/E forms), 9.61-9.81 (1H, two broad peaks, Z/E forms). MW 561.94. LCMS t_{R} (min): 1.94. MS (APCI+), m/z 562.10, 564.12 [M+H]⁺. HPLC t_{R} (min): 15.86. M_{P} 212-213° C.

### 31. N-(3-Chloro-4-fluoro-phenyl)-N′-[1-(dibenzofuran-2-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.60 (2H, m), 1.94 (2H, m), 2.65 (2H, m), 3.70 (3H, broad peak), 4.90 (2H, superposition of two q, J=7.5 Hz, Z/E forms), 7.19 (1H, m), 7.29 (1H, m), 7.48 (1H, t, J=8.5 Hz), 7.62 (1H, broad t, J=8.5 Hz), 7.63-7.68 (1H, two broad peaks, Z/E forms), 7.79 (1H, broad d, J=8.5 Hz, Z/E forms), 7.92 (2H, m), 7.93-8.07 (1H, two broad peaks, Z/E forms), 8.37 (1H, d, J=8.5 Hz), 8.63 (1H, broad peak), 9.58-9.78 (1H, two broad peaks, Z/E forms). MW 651.04391. LCMS t_{R} (min): 2.25. MS (APCI+), m/z 651.04, 653.01 [M+H]⁺. HPLC t_{R} (min): 18.73. M_{P} 213-214° C.

### 32. 1-(6-{4-[4-(3-Chloro-4-fluoro-phenylamino)-6-(2,2,2-trifluoro-ethoxy)-[1,3,5]triazin-2-ylamino]-piperidine-1-sulfonyl}-3,4-dihydro-2H-quinolin-1-yl)-ethanone

¹H-NMR (400 MHz, DMSO-D₆) δ_{H}: 1.59 (2H, m), 1.93 (4H, m), 2.23 (3H, s), 2.50 (2H, m), 2.84 (2H, broad peak, Z/E forms), 3.63 (2H, broad peak, Z/E forms), 3.77 (3H, broad peak, Z/E forms), 4.90 (2H, broad q, J=7.5 Hz), 7.28 (1H, broad peak, Z/E forms), 7.40 (2H, broad peak, Z/E forms), 7.52 (1H, broad m), 7.67-7.82 (1H, two broad peaks, Z/E forms), 7.85-7.95 (1H, two broad peaks, Z/E forms), 8.09 (1H, two broad peaks, Z/E forms), 9.57-9.78 (1H, two broad peaks, Z/E forms). MW 658.08. LCMS t_{R} (min): 1.99. MS (APCI+), m/z 658.66, 660.09 [M+H]⁺. HPLC t_{R} (min): 16.43. M_{P} 229-230° C.

### 33. 6-{4-[4-(3-Chloro-4-fluoro-phenylamino)-6-(2,2,2-trifluoro-ethoxy)-[1,3,5]triazin-2-ylamino]-piperidine-1-sulfonyl}-3,4-dihydro-1H-quinolin-2-one

¹H-NMR (400 MHz, DMSO-D₆) δ_{H}: 1.58 (2H, m), 1.92 (2H, m), 2.49 (4H, m), 2.99 (2H, broad), 3.60 (2H, m), 3.73 (1H, broad peak), 4.90 (2H, broad q, J=7.5 Hz), 7.05 (1H, d, J=8.5 Hz), 7.28 (1H, superposition of two t, J=8.5 Hz, Z/E forms), 7.35 (1H, broad peaks,), 7.52 (1H, d, J=8.5 Hz), 7.55 (1H, s), 7.67-7.84 (1H, two broad peaks, Z/E forms), 7.95-8.18 (1H, two broad peaks, Z/E forms), 9.60-9.80 (1H, two broad peaks, Z/E forms), 10.38-10.45 (1H, two broad peaks, Z/E forms). MW 630.02. LCMS t_{R} (min): 1.87. MS (APCI+), m/z 630.0; 632.0 [M+H]⁺. HPLC t_{R} (min): 14.89. M_{P} 289.4-290.3° C.

### 34. 1-(5-{4-[4-(3-Chloro-4-fluoro-phenylamino)-6-(2,2,2-trifluoro-ethoxy)-[1,3,5]triazin-2-ylamino]-piperidine-1-sulfonyl}-2,3-dihydro-indol-1-yl)-propan-1-one

¹H-NMR (400 MHz, DMSO-D₆) δ_{H}: 1.10 (3H, broad peak, Z/E forms), 1.58 (2H, m), 1.93 (2H, m), 2.40 (4H, m), 3.24 (2H, broad peak, Z/E forms), 3.60 (2H, m), 3.72 (1H, broad peak, Z/E forms), 4.18 (2H, broad q, J=7.5 Hz), 4.92 (2H, broad q, J=7.5 Hz), 7.28 (1H, superposition of two t, Z/E forms), 7.38-7.54 (1H, two broad peaks, Z/E forms), 7.57 (2H, broad peak, Z/E forms), 7.67-7.82 (1H, two broad peaks, Z/E forms), 7.94-8.14 (1H, two broad peaks, Z/E forms), 8.22 (1H, broad peak, Z/E forms), 9.60-9.80 (1H, two broad peaks, Z/E forms). MW 658.08. LCMS t_{R} (min): 2.03. MS (APCI+), m/z 658.66 [M+H]⁺. HPLC t_{R} (min): 16.64. M_{P} 245.3-245.9° C.

### 35. N-(3-Chloro-4-fluoro-phenyl)-N′-[1-(naphthalene-2-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.60 (2H, m), 1.95 (2H, m), 2.63 (2H, m), 3.70 (3H, two broad peaks, Z/E forms), 4.90 (2H, superposition of two q, J=7.5 Hz, Z/E forms), 7.23 (1H, broad peak), 7.31 (1H, t, J=8.5 Hz), 7.76 (4H, superposition of m), 7.94 (1H, broad peak, Z/E forms), 8.10 (1H, t, J=8.5 Hz), 8.21 (2H, superposition of m), 8.46 (1H, s), 9.68-9.85 (1H, two broad peaks, Z/E forms). MW 611.02. LCMS t_{R} (min): 2.18. MS (APCI+), m/z 611.11, 613.08 [M+H]⁺. HPLC t_{R} (min): 18.30. M_{P} 240.2-241.1° C.

### 36. N-(3-Chloro-4-fluoro-phenyl)-N′-[1-(naphthalene-1-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.52 (2H, m), 1.90 (2H, m), 2.75-2.80 (2H, two m, Z/E forms), 3.70 (2H, m), 3.71-3.88 (1H, two broad peaks), 4.88 (2H, broad q, J=7.5 Hz), 7.22 (1H, superposition of two m, Z/E forms), 7.24-7.38 (1H, two broad peaks, Z/E forms), 7.69 (4H, m), 7.93-8.04 (1H, two broad peaks, Z/E forms), 8.12 (2H, m), 8.28 (1H, t, J=8.5 Hz), 8.63-8.70 (1H, two d, J=8.5 Hz, Z/E forms), 9.58-9.75 (1H, two broad peaks, Z/E forms). MW 611.02. LCMS t_{R} (min): 2.16. MS (APCI), m/z 611.10, 613.08 [M+H]⁺. HPLC t_{R} (min): 18.12. M_{P} 208.1-209.0° C.

### 37. N-{2-[4-(3-Chloro-4-fluoro-phenylamino)-6-(2,2,2-trifluoro-ethoxy)-[1,3,5]triazin-2-ylamino]-ethyl}-benzenesulfonamide

¹H-NMR (400 MHz, DMSO-D₆) δ_{H}: 2.98 (2H, broad peak, Z/E forms), 3.37 (2H, broad peak, Z/E forms), 4.92 (2H, broad peak, Z/E forms), 7.31 (1H, m), 7.56 (2H, broad peak, Z/E forms), 7.62 (4H, two broad peaks, Z/E forms), 7.78 (2H, broad peak, Z/E forms), 7.89-7.99 (1H, two broad peaks, Z/E forms), 9.63-9.74 (1H, two broad peaks, Z/E forms). MW 520.89. LCMS t_{R} (min): 1.97. MS (APCI+), m/z 521.05, 523.06 [M+H]⁺. HPLC t_{R} (min): 16.25. M_{P} 194-196° C.

### 38. N-{3-[4-(3-Chloro-4-fluoro-phenylamino)-6-(2,2,2-trifluoro-ethoxy)-[1,3,5]-triazin-2-ylamino]-propyl}-benzenesulfonamide

¹H-NMR (400 MHz, DMSO-D₆) δ_{H}: 1.71 (2H, m), 2.89 (2H, broad peak, Z/E forms), 3.39 (2H, broad peak, Z/E forms), 4.95 (2H, broad q, J=7.5 Hz), 7.31 (1H, broad t), 7.61 (6H, superposition of broad peak, Z/E forms), 7.79 (2H, broad peak, Z/E forms), 8.01 (1H, broad t, J=7.5 Hz), 9.61-9.79 (1H, two broad peaks, Z/E forms). MW 534.92. LCMS t_{R} (min): 2.08. MS (APCI+), m/z 534.90, 536.95 [M+H]⁺. HPLC t_{R} (min): 16.44. M_{P} 189-190° C.

### 39. N-(3-Chloro-4-fluoro-phenyl)-N′-[1-(2,2-dimethyl-chroman-6-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.31 (6H, s), 1.61 (2H, m), 1.95 (2H, m), 2.01 (2H, m), 2.32 (2H, m), 2.79 (2H, broad peaks, Z/E forms), 3.61 (2H, m), 3.68 (1H, broad peak, Z/E forms), 4.92 (2H, broad q, J=7.5 Hz), 6.88 (1H, d, J=8.5 Hz), 7.21 (1H, superposition of two m, Z/E forms), 7.21-7.31 (1H, two broad peaks, Z/E forms), 7.38 (1H, d, J=8.5 Hz), 7.49 (1H, s), 7.65-7.71 (1H, two broad peaks, Z/E forms), 7.91-8.22 (1H, two broad peaks, Z/E forms), 9.51-9.51 (1H, two broad peaks, Z/E forms). MW 645.08. LCMS t_{R} (min): 2.22. MS (APCI+), m/z 644.97, 646.95 [M+H]⁺. HPLC t_{R} (min): 19.00. M_{P} 241-243° C.

### 40. N-(3-Chloro-4-fluoro-phenyl)-N′-[1-(3,4-dihydro-2H-benzo[b][1,4]dioxepine-7-sulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.58 (2H, m), 1.93 (2H, m), 2.20 (2H, m), 2.42 (2H, m), 3.62 (2H, m), 3.77 (1H, broad), 4.28 (4H, m), 4.92 (2H, broad q, J=7.5 Hz), 7.16 (1H, d, J=8.5 Hz), 7.25 (2H, broad peak, Z/E forms), 7.30 (1H, broad peak, Z/E forms), 7.30-7.38 (1H, two broad peaks, Z/E forms), 7.67-7.83 (1H, two broad peaks, Z/E forms), 7.95-8.18 (1H, two broad peaks, Z/E forms), 9.58-9.80 (1H, two broad peaks, Z/E forms). MW 633.02. LCMS t_{R} (min): 2.12. MS (APCI+), m/z 633.06, 635.05 [M+H]⁺. HPLC t_{R} (min): 17.60. M_{P} 213-215° C.

### Generic Synthesis of Intermediates and Final Compounds for Table 38

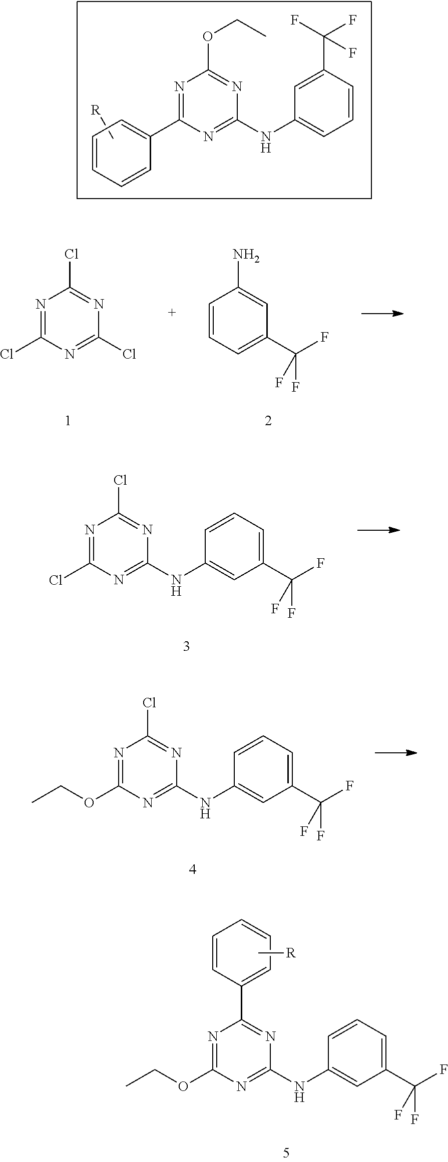

Intermediate 3. To a solution of 2,4,6-trichlorotriazine 1 (50 g, 271 mmol) in dioxane was added CH₃COONa (1 equiv., 22.23 g, 271 mmol) at 0-5° C. Then 3-trifluoromethylaniline 2 (1 equiv., 43.67 g, 271 mmol) was added dropwise on stirring while the reaction temperature was maintained below 5° C. The reaction mixture was stirred for 1 h. at r.t. and concentrated under reduced pressure. The residue was washed with CHCl₃, filtered, and concentrated under reduced pressure. The precipitate was washed with hexane. Yield 42 g. (52%).

Intermediate 4. Sodium (0.99 g, 43 mmol) was carefully dissolved in dry ethanol (100 ml) at r.t. 2,4-Dichloro-6-(N-3-trifluoromethyl-anilino)-1,3,5-triazine 3 (13.2 g, 43 mmol) was dissolved separately in dry ethanol (50 ml), cooled to −10° C. and treated with the sodium ethoxide solution. The reaction mixture was stirred for 3 hours at r.t. LCMS analysis of the reaction mixture demonstrated no initial 2,4-dichloro-6-(N-3-trifluoromethyl-anilino)-1,3,5-triazine. The main product of this reaction was 2-ethoxy-4-chloro-6-(N-3-trifluoromethyl-anilino)-1,3,5-triazine 4 (M+1=319.4) in 90% yield. Minor components were 2,4-diethoxy-6-(N-3-trifluoromethylanilino)-1,3,5-triazine and 2,4-dioxy-6-(N-3-trifluoromethyl-anilino)-1,3,5-triazine (M+1=329.0 and M+1=273.0, correspondingly), 10% of total. The reaction mixture was concentrated and the crude product 4 was re-crystallized from hexane. Yield 5.5 g. (40%).

Compound 5 was synthesized according to the following procedure: 4 (10 g, 27 mmol), 2 (10.82 g, 32 mmol), and triphenylphosphine (1.05 g, 4 mmol) were charged into the flask containing 200 mL of dioxane and 30 mL of aqueous 2 M Na₂CO₃. After purging the mixture with Ar for 20 min, [Pd(PPh₃)₄] catalyst (5 mol %) was added. The reaction mixture was heated at 100 □C for 6 h under an Ar atmosphere. After cooling the reaction mixture to rt, the solvent was removed under reduced pressure to produce a yellow oil. The residue was washed with water (20 mL) and extracted with CHCl₃ (20 mL). The organic layer was isolated, and the remaining aqueous portion was extracted again with CHCl₃ (3×20 mL). The organic extracts were combined, and the solvent was removed in vacuo. The crude product was purified by silica gel chromatography (ca. 120 mL) using CHCl₃-hexanes (3:1 v/v) as an eluent.

TABLE 38

| En | Structure | IUPAC Name | MW | Formula |
|---|---|---|---|---|
| 1 | 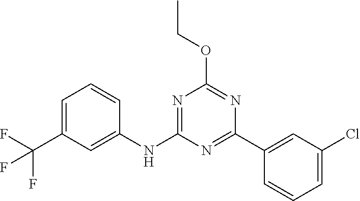 | 4-(3-chlorophenyl)-6-ethoxy-N-[3- (trifluoromethyl)phenyl]-1,3,5- triazin-2-amine | 394.8 | C18H14ClF3N4O |
| 2 | 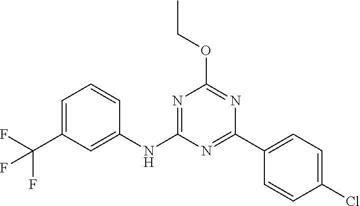 | 4-(4-chlorophenyl)-6-ethoxy-N-[3- (trifluoromethyl)phenyl]-1,3,5- triazin-2-amine | 394.8 | C18H14ClF3N4O |
| 3 | 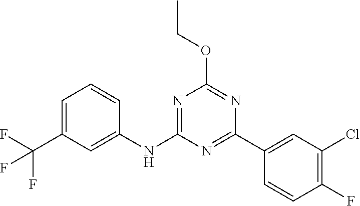 | 4-(3-chloro-4-fluorophenyl)-6- ethoxy-N-[3- (trifluoromethyl)phenyl]-1,3,5- triazin-2-amine | 412.8 | C18H13ClF4N4O |
| 4 | 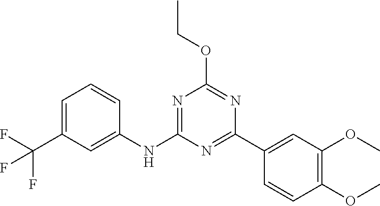 | 4-(3,4-dimethoxyphenyl)-6-ethoxy- N-[3-(trifluoromethyl)phenyl]-1,3,5- triazin-2-amine | 420.4 | C20H19F3N4O3 |
| 5 | 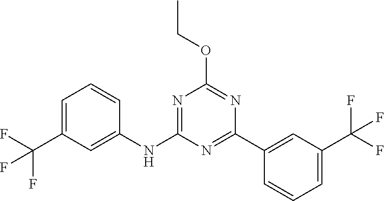 | 4-ethoxy-N,6-bis[3- (trifluoromethyl)phenyl]-1,3,5- triazin-2-amine | 428.3 | C19H14F6N4O |
| 6 | 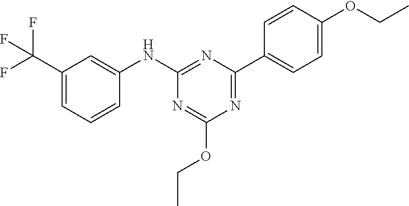 | 4-ethoxy-6-(4-ethoxyphenyl)-N-[3- (trifluoromethyl)phenyl]-1,3,5- triazin-2-amine | 404.4 | C20H19F3N4O2 |
| 7 | 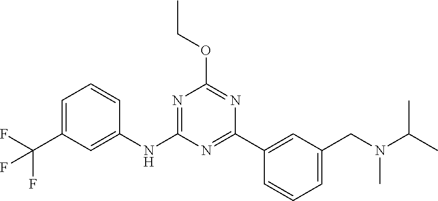 | 4-ethoxy-6-(3-{[methyl(propan-2- yl)amino]methyl}phenyl)-N-[3- (trifluoromethyl)phenyl]-1,3,5- triazin-2-amine | 445.5 | C23H26F3N5O |
| 8 | 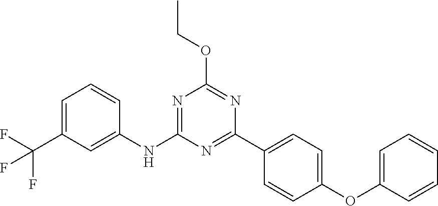 | 4-ethoxy-6-(4-phenoxyphenyl)-N- [3-(trifluoromethyl)phenyl]-1,3,5- triazin-2-amine | 452.4 | C24H19F3N4O2 |
| 9 | 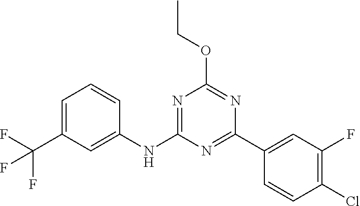 | 4-(4-chloro-3-fluorophenyl)-6- ethoxy-N-[3- (trifluoromethyl)phenyl]-1,3,5- triazin-2-amine | 412.8 | C18H13ClF4N4O |
| 10 | 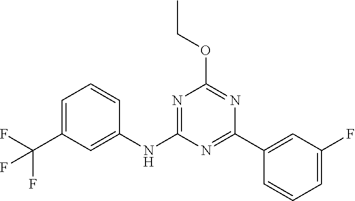 | 4-ethoxy-6-(3-fluorophenyl)-N-[3- (trifluoromethyl)phenyl]-1,3,5- triazin-2-amine | 378.3 | C18H14F4N4O |
| 11 | 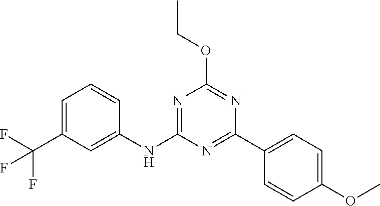 | 4-ethoxy-6-(4-methoxyphenyl)-N- [3-(trifluoromethyl)phenyl]-1,3,5- triazin-2-amine | 390.4 | C19H17F3N4O2 |
| 12 | 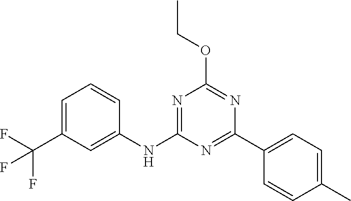 | 4-ethoxy-6-(4-methylphenyl)-N-[3- (trifluoromethyl)phenyl]-1,3,5- triazin-2-amine | 374.4 | C19H17F3N4O |
| 13 | 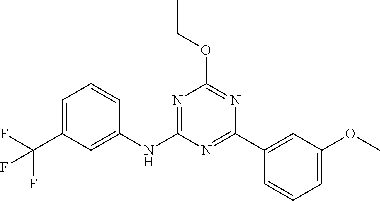 | 4-ethoxy-6-(3-methoxyphenyl)-N- [3-(trifluoromethyl)phenyl]-1,3,5- triazin-2-amine | 390.4 | C19H17F3N4O2 |
| 14 | 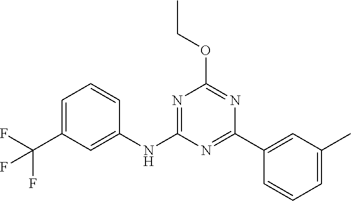 | 4-ethoxy-6-(3-methylphenyl)-N-[3- (trifluoromethyl)phenyl]-1,3,5- triazin-2-amine | 374.4 | C19H17F3N4O |
| 15 | 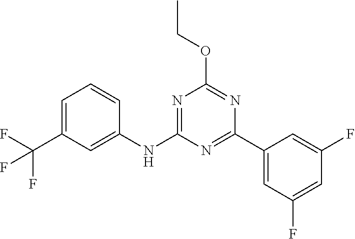 | 4-(3,5-difluorophenyl)-6-ethoxy-N- [3-(trifluoromethyl)phenyl]-1,3,5- triazin-2-amine | 396.3 | C18H13F5N4O |
| 16 | 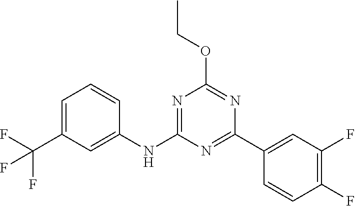 | 4-(3,4-difluorophenyl)-6-ethoxy-N- [3-(trifluoromethyl)phenyl]-1,3,5- triazin-2-amine | 396.3 | C18H13F5N4O |
| 17 | 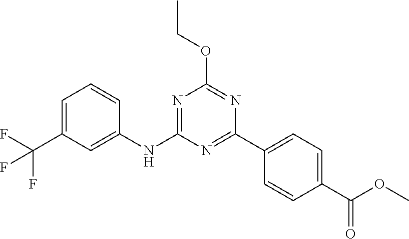 | methyl 4-(4-ethoxy-6-{[3- (trifluoromethyl)phenyl]amino}- 1,3,5-triazin-2-yl)benzoate | 418.4 | C20H17F3N4O3 |
| 18 | 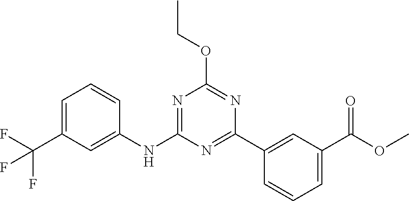 | methyl 3-(4-ethoxy-6-{[3- (trifluoromethyl)phenyl]amino}- 1,3,5-triazin-2-yl)benzoate | 418.4 | C20H17F3N4O3 |
| 19 | 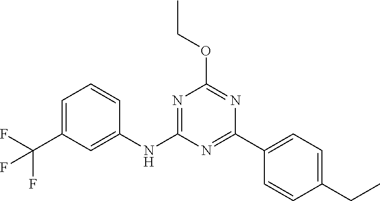 | 4-ethoxy-6-(4-ethylphenyl)-N-[3- (trifluoromethyl)phenyl]-1,3,5- triazin-2-amine | 388.4 | C20H19F3N4O |
| 20 | 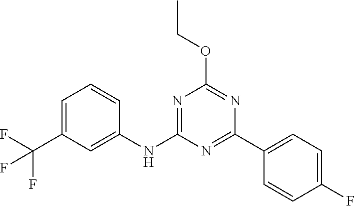 | 4-ethoxy-6-(4-fluorophenyl)-N-[3- (trifluoromethyl)phenyl]-1,3,5- triazin-2-amine | 378.3 | C18H14F4N4O |
| 21 | 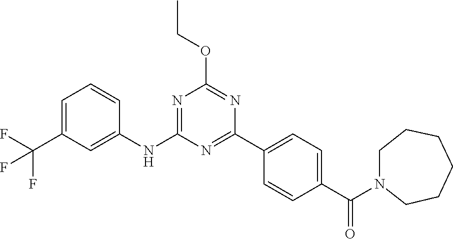 | azepan-1-yl[4-(4-ethoxy-6-{[3- (trifluoromethyl)phenyl]amino}- 1,3,5-triazin-2- yl)phenyl]methanone | 485.5 | C25H26F3N5O2 |
| 22 | 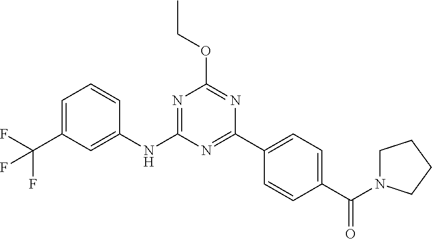 | [4-(4-ethoxy-6-{[3- (trifluoromethyl)phenyl]amino}- 1,3,5-triazin-2-yl)phenyl](pyrrolidin- 1-yl)methanone | 457.4 | C23H22F3N5O2 |
| 23 | 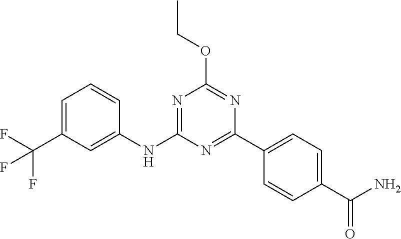 | 4-(4-ethoxy-6-{[3- (trifluoromethyl)phenyl]amino}- 1,3,5-triazin-2-yl)benzamide | 403.4 | C19H16F3N5O2 |
| 24 | 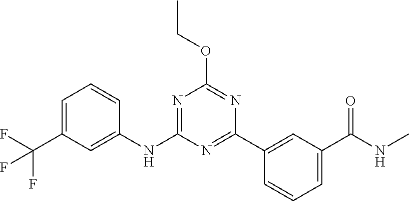 | 3-(4-ethoxy-6-{[3- (trifluoromethyl)phenyl]amino}- 1,3,5-triazin-2-yl)-N- methylbenzamide | 417.4 | C20H18F3N5O2 |
| 25 | 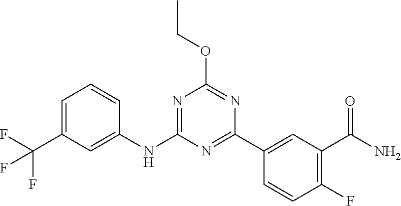 | 5-(4-ethoxy-6-{[3- (trifluoromethyl)phenyl]amino}- 1,3,5-triazin-2-yl)-2- fluorobenzamide | 421.3 | C19H15F4N5O2 |
| 26 | 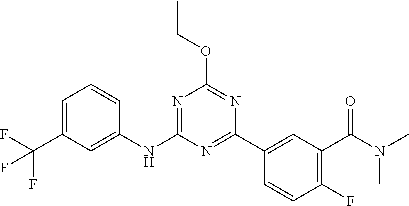 | 5-(4-ethoxy-6-{[3- (trifluoromethyl)phenyl]amino}- 1,3,5-triazin-2-yl)-2-fluoro-N,N- dimethylbenzamide | 449.4 | C21H19F4N5O2 |
| 27 | 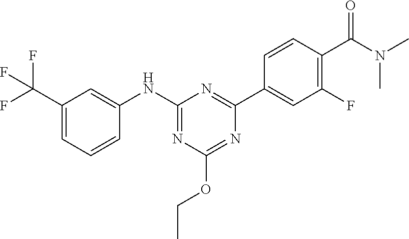 | 4-(4-ethoxy-6-{[3- (trifluoromethyl)phenyl]amino}- 1,3,5-triazin-2-yl)-2-fluoro-N,N- dimethylbenzamide | 449.4 | C21H19F4N5O2 |
| 28 | 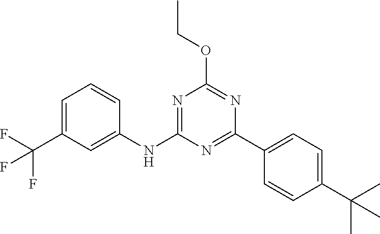 | 4-(4-tert-butylphenyl)-6-ethoxy-N- [3-(trifluoromethyl)phenyl]-1,3,5- triazin-2-amine | 416.4 | C22H23F3N4O |
| 29 | 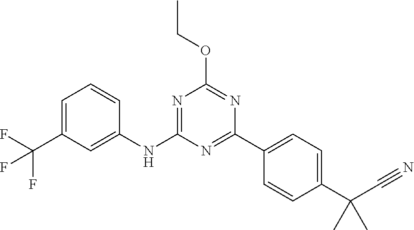 | 2-[4-(4-ethoxy-6-{[3- (trifluoromethyl)phenyl]amino}- 1,3,5-triazin-2-yl)phenyl]-2- methylpropanenitrile | 427.4 | C22H20F3N5O |
| 30 | 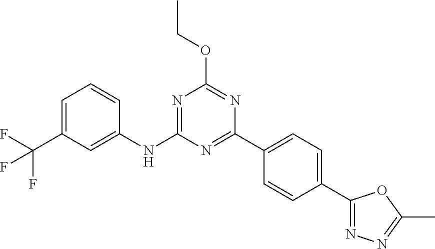 | 4-ethoxy-6-[4-(5-methyl-1,3,4- oxadiazol-2-yl)phenyl]-N-[3- (trifluoromethyl)phenyl]-1,3,5- triazin-2-amine | 442.4 | C21H17F3N6O2 |
| 31 | 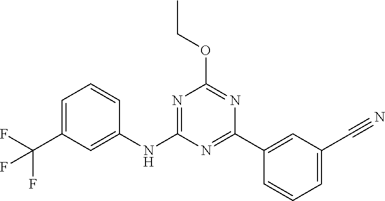 | 3-(4-ethoxy-6-{[3- (trifluoromethyl)phenyl]amino}- 1,3,5-triazin-2-yl)benzonitrile | 385.3 | C19H14F3N5O |
| 32 | 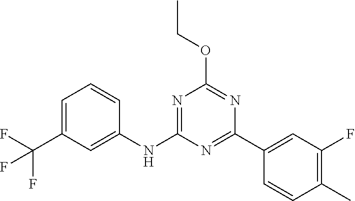 | 4-ethoxy-6-(3-fluoro-4- methylphenyl)-N-[3- (trifluoromethyl)phenyl]-1,3,5- triazin-2-amine | 392.4 | C19H16F4N4O |
| 33 | 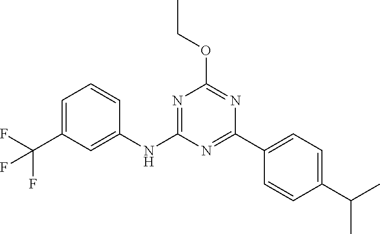 | 4-ethoxy-6-[4-(propan-2-yl)phenyl]- N-[3-(trifluoromethyl)phenyl]-1,3,5- triazin-2-amine | 402.4 | C21H21F3N4O |
| 34 | 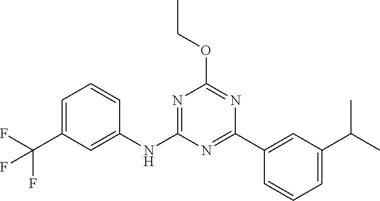 | 4-ethoxy-6[3-(propan-2-yl)phenyl]- N-[3-(trifluoromethyl)phenyl]-1,3,5- triazin-2-amine | 402.4 | C21H21F3N4O |
| 35 | 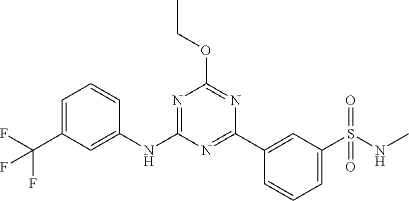 | 3-(4-ethoxy-6-{[3- (trifluoromethyl)phenyl]amino}- 1,3,5-triazin-2-yl)-N- methylbenzenesulfonamide | 454.4 | C19H18F3N5O3S |
| 36 | 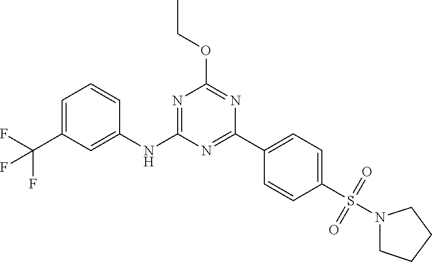 | 4-ethoxy-6-[4-(pyrrolidin-1- ylsulfonyl)phenyl]-N-[3- (trifluoromethyl)phenyl]-1,3,5- triazin-2-amine | 494.5 | C22H22F3N5O3S |
| 37 | 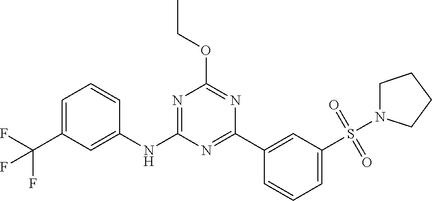 | 4-ethoxy-6-[4-(pyrrolidin-1- ylsulfonyl)phenyl]-N-[3- (trifluoromethyl)phenyl]-1,3,5- triazin-2-amine | 494.5 | C22H22F3N5O3S |
| 38 | 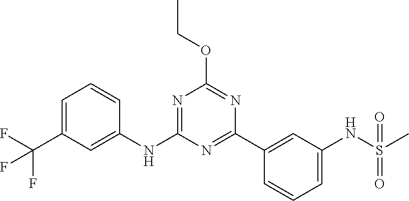 | N-[3-(4-ethoxy-6-{[3- (trifluoromethyl)phenyl]amino}- 1,3,5-triazin-2- yl)phenyl]methanesulfonamide | 454.4 | C19H18F3N5O3S |
| 39 | 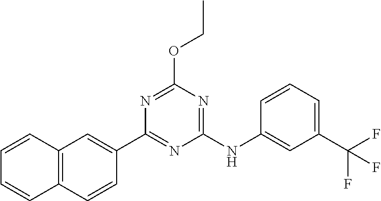 | 4-ethoxy-6-(naphthalen-2-yl)-N-[3- (trifluoromethyl)phenyl]-1,3,5- triazin-2-amine | 410.4 | C22H17F3N4O |

### Procedures and Analytical Data for Table 38.

An compounds were prepared by the above procedures.

### 1. 4-(3-chlorophenyl)-6-ethoxy-N-[3-(trifluoromethyl)phenyl]-1,3,5-triazin-2-amine

LCMS: M+1=394.7; 1H NMR (DMSO-d6, 90° C., ppm): d=1.48 t (3H), 4.56 q (2H), 7.46 m (4H), 7.66 d (1H), 7.84 s (1H), 8.36 m (3H).

### 2. 4-(4-chlorophenyl)-6-ethoxy-N-[3-(trifluoromethyl)phenyl]-1,3,5-triazin-2-amine

LCMS: M+1=394.7; 1H NMR (DMSO-d6, 90° C., ppm): d=1.48 t (3H), 4.56 q (2H), 7.44 m (4H), 7.68 d (1H), 7.94 s (1H), 8.32 m (3H).

### 3. 4-(3-chloro-4-fluorophenyl)-6-ethoxy-N-[3-(trifluoromethyl)phenyl]-1,3,5-triazin-2-amine

LCMS: M+1=412.7; 1H NMR (DMSO-d6, 90° C., ppm): d=1.46 t (3H), 4.54 q (2H), 7.40 d (1H), 7.54 m (2H), 7.94 d (1H), 8.34 m (2H), 8.46 d (1H), 10.02 s (1H).

### 4. 4-(3,4-dimethoxyphenyl)-6-ethoxy-N-[3-(trifluoromethyl)phenyl]-1,3,5-triazin-2-amine

LCMS: M+1=420.3; 1H NMR (DMSO-d6, 90° C., ppm): d=1.42 t (3H), 3.88 s (6H), 4.92 q (2H), 7.12 d (1H), 7.42 d (1H), 7.58 t (1H), 8.02 m (3H), 8.36 s (1H), 10.02 s (1H).

### 5. 4-ethoxy-N,6-bis[3-(trifluoromethyl)phenyl]-1,3,5-triazin-2-amine

LCMS: M+1=429.4 NMR 1H, DMSO-d6 δ, ppm: 1.42 t (3H); 4.55 m (2H); 7.41 d (1H); 7.59 t (1H); 7.79 t (1H); 7.94 d (2H); 8.38 s (1H); 8.64 d (2H); 10.30 s (1H).

### 6. 4-ethoxy-6-(4-ethoxyphenyl)-N-[3-(trifluoromethyl)phenyl]-1,3,5-triazin-2-amine

LCMS: M+1=404.3; 1H NMR (DMSO-d6, 90° C., ppm): d=1.38 t (6H), 4.20 q (2H), 4.52 q (2H), 7.08 t (2H), 7.36 d (1H), 7.58 t (1H), 7.92 d (1H), 8.34 m (1H), 10.08 s (1H).

### 7. 4-ethoxy-6-(3-{[methyl(propan-2-yl)amino]methyl}phenyl)-N-[3-(trifluoromethyl)phenyl]-1,3,5-triazin-2-amine

LCMS: M+1=445.4; 1H NMR (DMSO-d6, 90° C., ppm): d=1.20 m (6H), 1.42 t (3H), 2.40 m (3H), 3.60 m (2H), 4.54 q (2H), 7.40 d (1H), 7.64 m (3H), 7.82 d (1H), 8.42 m (3H), 10.08 s (1H).

### 8. 4-ethoxy-6-(4-phenoxyphenyl)-N-[3-(trifluoromethyl)phenyl]-1,3,5-triazin-2-amine

LCMS: M+1=453.3 NMR 1H, DMSO-d6 δ, ppm: 1.41 t (3H); 4.52 m (2H); 7.12 m (4H); 7.22 t (1H); 7.42 m (3H); 7.58 t (1H); 8.00 d (1H); 8.36 t (3H); 10.15 s (1H).

### 9. 4-(4-chloro-3-fluorophenyl)-6-ethoxy-N-[3-(trifluoromethyl)phenyl]-1,3,5-triazin-2-amine

LCMS: M+1=412.9; 1H NMR, DMSO-d6 δ, ppm: 1.41 t (3H); 4.55 m (2H); 7.40 d (1H); 7.58 t (1H); 7.73 t (1H); 7.96 d (1H); 8.17 d (2H); 8.31 s (1H); 10.30 s (1H).

### 10. 4-ethoxy-6-(3-fluorophenyl)-N-[3-(trifluoromethyl)phenyl]-1,3,5-triazin-2-amine

LCMS: M+1=378.3; 1H NMR (DMSO-d6, 90° C., ppm): 1=1.41 t (3H), 4.52q (2H), 7.41 m (2H), 7.59 m (2H), 8.01 m (2H), 8.21 d (1H), 8.38 s (1H), 10.15 s (1H).

### 11. 4-ethoxy-6-(4-methoxyphenyl)-N-[3-(trifluoromethyl)phenyl]-1,3,5-triazin-2-amine

LCMS: M+1=391.4; 1H NMR, DMSO-d6 δ, ppm: 1.42 t (3H); 3.88 s (3H); 4.52 m (2H); 7.08 d (2H); 7.37 d (1H); 7.57 t (1H); 7.98 d (1H); 8.34 d (3H); 10.09 s (1H).

### 12. 4-ethoxy-6-(4-methylphenyl)-N-[3-(trifluoromethyl)phenyl]-1,3,5-triazin-2-amine

LCMS: M+1=375.4; 1H NMR, DMSO-d6 δ, ppm: 1.41 t (3H); 2.40 s (3H); 4.54 m (2H); 7.36 t (3H); 7.58 t (1H); 7.98 d (1H); 8.28 d (2H); 8.38 s (1H); 10.12 s (1H).

### 13. 4-ethoxy-6-(3-methoxyphenyl)-N-[3-(trifluoromethyl)phenyl]-1,3,5-triazin-2-amine

LCMS: M+1=391.4; 1H NMR, DMSO-d6 δ, ppm: 1.41 t (3H); 3.88 s (3H); 4.55 m (2H); 7.18 dd (1H); 7.43 m (2H); 7.57 t (1H); 7.92 t (1H); 7.97 d (2H); 8.37 s (1H); 10.17 (1H).

### 14. 4-ethoxy-6-(3-methylphenyl)-N-[3-(trifluoromethyl)phenyl]-1,3,5-triazin-2-amine

LCMS: M+1=375.4; 1H NMR, DMSO-d6 δ, ppm: 1.43 t (3H); 4.55 m (2H); 7.41 m (3H); 7.58 t (1H); 7.95 d (1H); 8.20 m (2H); 8.45 s (1H); 10.10 bs (1H).

### 15. 4-(3,5-difluorophenyl)-6-ethoxy-N-[3-(trifluoromethyl)phenyl]-1,3,5-triazin-2-amine

LCMS: M+1=396.3; 1H NMR (DMSO-d6, 90° C., ppm): 1=1.48 t (3H), 4.55q (2H), 7.40 m (2H), 7.58 t (1H), 7.91 m (3H), 8.34 s (1H), 10.19 s (1H).

### 16. 4-(3,4-difluorophenyl)-6-ethoxy-N-[3-(trifluoromethyl)phenyl]-1,3,5-triazin-2-amine

LCMS: M+1=397.3; 1H NMR, DMSO-d6 δ, ppm: 1.41 t (3H); 4.55 m (2H); 7.41 d (1H); 7.59 m (2H); 7.96 d (1H); 8.22 m (2H); 8.33 s (1H); 10.24 s (1H).

### 17. methyl 4-(4-ethoxy-6-{[3-(trifluoromethyl)phenyl]amino}-1,3,5-triazin-2-yl)benzoate

LCMS: M+1=419.4; 1H NMR, DMSO-d6 δ, ppm: 1.41 t (3H); 3.93 s (3H); 4.55 m (2H); 7.41 d (1H); 7.60 t (1H); 7.99 d (1H); 8.11 d (2H); 8.34 s (1H); 8.47 d (2H); 10.27 s (1H).

### 18. methyl 3-(4-ethoxy-6-{[3-(trifluoromethyl)phenyl]amino}-1,3,5-triazin-2-yl)benzoate

LCMS: M+1=419.4; 1H NMR, DMSO-d6 δ, ppm: 1.43 t (3H); 3.93 s (3H); 4.55 m (2H); 7.41 d (1H); 7.57 t (1H); 7.69 t (1H); 7.98 d (1H); 8.16 m (1H); 8.35 s (1H); 8.59 m (1H); 8.95 t (1H); 10.23 s (1H).

### 19. 4-ethoxy-6-(4-ethylphenyl)-N-[3-(trifluoromethyl)phenyl]-1,3,5-triazin-2-amine

LCMS: M+1=389.3; 1H NMR, DMSO-d6 δ, ppm: 1.25 t (3H); 1.42 t (3H); 2.73 m (2H); 4.55 m (2H); 7.38 bd (3H); 7.57 t (1H); 7.99 d (1H); 8.30 d (2H); 8.38 s (1H); 10.07 s (1H).

### 20. 4-ethoxy-6-(4-fluorophenyl)-N-[3-(trifluoromethyl)phenyl]-1,3,5-triazin-2-amine

LCMS: M+1=379.4; 1H NMR, DMSO-d6 δ, ppm: 1.41 t (3H); 4.55 m (2H); 7.34 m (3H); 7.58 t (1H); 8.00 d (1H); 8.34 s (1H); 8.44 m (2H); 10.25 s (1H).

### 21. azepan-1-yl[4-(4-ethoxy-6-{[3-(trifluoromethyl)phenyl]amino}-1,3,5-triazin-2-yl)phenyl]methanone

LCMS: M+1=486.4; 1H NMR, DMSO-d6 δ, ppm: 1.42 t (3H); 1.65 m (8H); 3.49 m (4H); 4.55 m (2H); 7.41 d (1H); 7.51 d (2H); 7.59 t (1H); 8.01 d (1H); 8.34 s (1H); 8.43 d (2H); 10.17 s (1H).

### 22. [4-(4-ethoxy-6-{[3-(trifluoromethyl)phenyl]amino}-1,3,5-triazin-2-yl)phenyl](pyrrolidin-1-yl)methanone

LCMS: M+1=458.4; 1H NMR, DMSO-d6 δ, ppm: 1.42 t (3H); 1.88 t (4H); 3.46 t (4H); 4.55 m (2H); 7.41 d (1H); 7.63 m (3H); 8.01 d (1H); 8.34 s (1H); 8.43 d (2H); 10.17 s 1H).

### 23. 4-(4-ethoxy-6-{[3-(trifluoromethyl)phenyl]amino}-1,3,5-triazin-2-yl)benzamide

LCMS: M+1=404.4; 1H NMR, DMSO-d6 δ, ppm: 1.41 t (3H); 4.55 m (2H); 7.50 bm (4H); 8.02 bt (3H); 8.41 bt (3H); 10.27 s (1H).

### 24. 3-(4-ethoxy-6-{[3-(trifluoromethyl)phenyl]amino}-1,3,5-triazin-2-yl)-N-methylbenzamide

LCMS: M+1=418.4; 1H NMR, DMSO-d6 δ, ppm: 1.42 t (3H); 2.82 d (3H); 4.55 m (2H); 7.39; d (1H); 7.57 m (2H); 8.02 d (2H); 8.21 bs (1H); 8.34 s (1H); 8.46 d (1H); 8.82 s (1H); 10.20 s (1H).

### 25. 5-(4-ethoxy-6-{[3-(trifluoromethyl)phenyl]amino}-1,3,5-triazin-2-yl)-2-fluorobenzamide

LCMS: M+1=422.4; 1H NMR, DMSO-d6 δ, ppm: 1.41 t (3H); 4.55 m (2H); 7.45 m (5H); 7.98 d (1H); 8.31 bs (1H); 8.46 bm (1H); 8.74 d (1H); 10.31 bs (1H).

### 26. 5-(4-ethoxy-6-{[3-(trifluoromethyl)phenyl]amino}-1,3,5-triazin-2-yl)-2-fluoro-N,N-dimethylbenzamide

LCMS: M+1=450.4; 1H NMR, DMSO-d6 δ, ppm: 1.42 t (3H); 3.03 s (6H); 4.55 m (2H); 7.41; m (2H); 7.57 t (1H); 7.95 d (1H); 8.34 m (2H); 8.43 m (1H); 10.17 s (1H).

### 27. 4-(4-ethoxy-6-{[3-(trifluoromethyl)phenyl]amino}-1,3,5-triazin-2-yl)-2-fluoro-N,N-dimethylbenzamide

LCMS: M+1=450.45; 1H NMR, DMSO-d6 δ, ppm: 1.41 t (3H); 3.00 s (6H); 4.55 m (2H); 7.40 d (1H); 7.58 m (2H); 7.97 d (1H); 8.10 d (1H); 8.25 d (1H); 8.35 s (1H); 10.30 s (1H).

### 28. 4-(4-tert-butylphenyl)-6-ethoxy-N-[3-(trifluoromethyl)phenyl]-1,3,5-triazin-2-amine

LCMS: M+1=417.5; 1H NMR, DMSO-d6 δ, ppm: 1.37 s (9H); 1.42 t (3H); 4.55 m (2H); 7.39 d (1H); 7.57 t (3H); 7.99 d (1H); 8.31 t (3H); 10.07 s (1H).

### 29. 2-[4-(4-ethoxy-6-{[3-(trifluoromethyl)phenyl]amino}-1,3,5-triazin-2-yl)phenyl]-2-methylpropanenitrile

LCMS: M+1=428.5; 1H NMR, DMSO-d6 δ, ppm: 1.42 t (3H); 1.77 s (6H); 4.55 m (2H); 7.39 d (1H); 7.57 t (1H); 7.70 d (2H); 8.00 d (1H); 8.34 s (1H); 8.42 d (2H); 10.22 s 1H).

### 30. 4-ethoxy-6-[4-(5-methyl-1,3,4-oxadiazol-2-yl)phenyl]-N-[3-(trifluoromethyl)phenyl]-1,3,5-triazin-2-amine

LCMS: M+1=443.4; 1H NMR, DMSO-d6 δ, ppm: 1.41 t (3H); 2.61 s (3H); 4.55 m (2H); 7.41 d (1H); 7.60 t (1H); 7.99 d (1H); 8.10 d (211); 8.34 s (1H); 8.54 d (2H); 10.27 s (1H).

### 31. 3-(4-ethoxy-6-{[3-(trifluoromethyl)phenyl]amino}-1,3,5-triazin-2-yl)benzonitrile

LCMS: M+1=386.4; 1H NMR, DMSO-d6 δ, ppm: 1.41 t (3H); 4.55 m (2H); 7.41 d (1H); 7.60 t (1H); 7.77 t (1H); 7.97 d (1H); 8.03 d (1H); 8.34 s (1H); 8.63 d (2H); 10.25 s (1H).

### 32. 4-ethoxy-6-(3-fluoro-4-methylphenyl)-N-[3-(trifluoromethyl)phenyl]-1,3,5-triazin-2-amine

LCMS: M+1=393.3; 1H NMR, DMSO-d6 δ, ppm: 1.41 t (3H); 2.34 s (3H); 4.55 m (2H); 7.41 m (2H); 7.58 t (1H); 7.96 m (2H); 8.10 d (1H); 8.37 s (1H); 10.20 s (1H).

### 33. 4-ethoxy-6-[4-(propan-2-yl)phenyl]-N-[3-(trifluoromethyl)phenyl]-1,3,5-triazin-2-amine

LCMS: M+1=403.5; 1H NMR, DMSO-d6 δ, ppm: 1.27 d (6H) 1.42 t (3H); 3.00 m (1H); 4.55 m (2H); 7.41 t (3H); 7.60 t (1H); 7.99 d (1H); 8.31 d (2H); 8.39 s (1H); 10.12 s 1H).

### 34. 4-ethoxy-6-[3-(propan-2-yl)phenyl]-N-[3-(trifluoromethyl)phenyl]-1,3,5-triazin-2-amine

LCMS: M+1=403.4; 1H NMR, DMSO-d6 δ, ppm: 1.30 d (6H); 1.42 t (3H); 3.05 m (1H); 4.55 m (2H); 7.48 m (4H); 7.97 d (1H); 8.21 m (1H); 8.27 t (1H); 8.39 s (1H); 10.10 s 1H).

### 35. 3-(4-ethoxy-6-{[3-(trifluoromethyl)phenyl]amino}-1,3,5-triazin-2-yl)-N-methylbenzenesulfonamide

LCMS: M+1=454.4; 1H NMR, DMSO-d6 δ, ppm: 1.43 t (3H); 2.54 d (3H); 4.56 m (2H); 7.26 m (1H); 7.41 d (1H); 7.59 t (1H); 7.78 t (1H); 8.02 d (2H); 8.28 s (1H); 8.59 d (1H); 8.80 s (1H); 10.28 bs (1H).

### 36. 4-ethoxy-6-[4-(pyrrolidin-1-ylsulfonyl)phenyl]-N-[3-(trifluoromethyl)phenyl]-1,3,5-triazin-2-amine

LCMS: M+1=494.4; 1H NMR, DMSO-d6 δ, ppm: 1.43 t (3H); 1.72 m (4H); 3.24 m (4H); 4.56 m (2H); 7.41 d (1H); 7.60 t (1H); 7.98 m (3H); 8.30 s (1H); 8.55 d (2H); 10.30 s (1H).

### 37. 4-ethoxy-6-[4-(pyrrolidin-1-ylsulfonyl)phenyl]-N-[3-(trifluoromethyl)phenyl]-1,3,5-triazin-2-amine

LCMS: M+1=494.4; 1H NMR, DMSO-d6 δ, ppm: 1.43 t (3H); 1.75 m (4H); 3.25 m (4H); 4.56 m (2H); 7.41 d (1H); 7.58 t (1H); 7.82 t (1H); 8.03 d (2H); 8.28 s (1H); 8.63 d (1H); 8.77 t (1H); 10.30 bs (1H).

### 38. N-[3-(4-ethoxy-6-{[3-(trifluoromethyl)phenyl]amino}-1,3,5-triazin-2-yl)phenyl]methanesulfonamide

LCMS: M+1=454.4; 1H NMR, DMSO-d6 δ, ppm: 1.41 t (3H); 2.72 d (3H) 4.55 m (2H); 7.50 m (4H); 7.82 s (1H); 8.21 d (3H).

### 39. 4-ethoxy-6-(naphthalen-2-yl)-N-[3-(trifluoromethyl)phenyl]-1,3,5-triazin-2-amine

LCMS: M+1=411.6; 1H NMR, DMSO-d6 δ, ppm: 1.45 t (3H); 4.58 m (2H); 7.44 d (1H); 7.62 m (3H); 8.02 m (4H); 8.42 m (2H); 9.00 s (1H); 10.18 bs (1H).

### Generic Synthesis of Intermediates and Final Compounds for Table 39

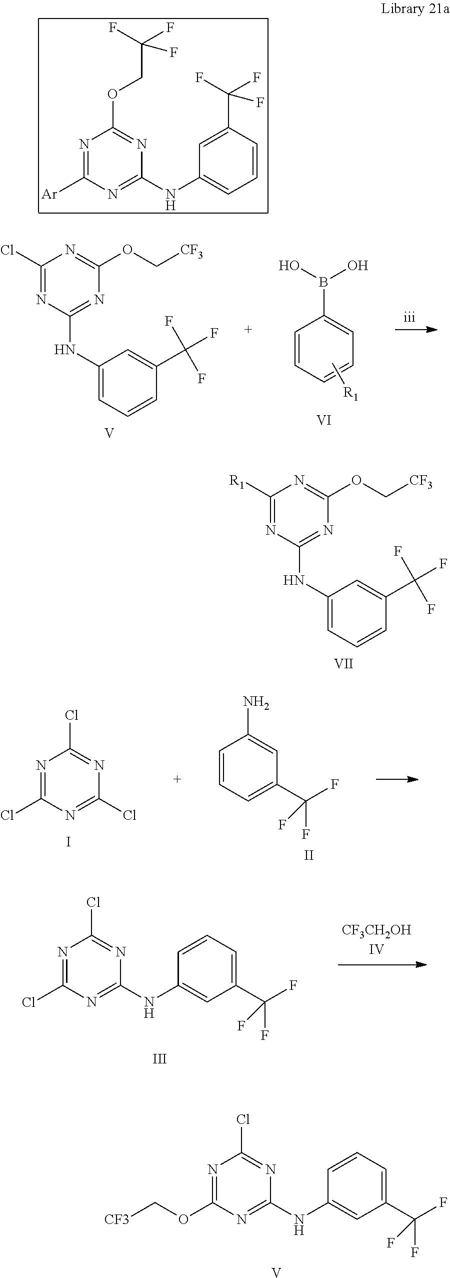

Preparation of 4-chloro-6-trifluoroethoxy-N-[3-(trifluoromethyl)phenyl]-1,3,5-triazine-2-amine V. To a suspension of 0.500 g (2.781 mmol) I, 0.225 g (2.781 mmol) of NaHCO₃ and 0.820 g (5.788 mmol) of Na₂SO₄ in 20 ml of anhydrous acetonitrile at −10° C. was added dropwise a solution of 3-(trifluoromethyl)aniline II in 10 ml of dry acetonitrile, 0.450 g (2.781 mmol), over 2 h. After complete addition, the cooling bath was removed and the mixture was stirred at rt for 3 h. The resulting precipitate was filtered, and the pale yellow solution of compound III (88% LCMS) was used in the next step without further purification.

To a solution of compound III in 30 ml anhydrous acetonitrile was added dropwise a cooled solution of potassium tert-butoxide (0.312 g, 2.781 mmol) in 2,2,2-trifluoro-1-ethanol (5 ml) over 2 h. After stirring this reaction mixture overnight at rt, the solid precipitate was filtered and washed with anhydrous acetonitrile (2×30 ml). The solvent was removed in vacuo to afford a yellow oil. To this oil was added (3×50 mL) anhydrous hexanes and the mixture was heated at reflux. After 1 min the hexane layer was decanted. The solvent was removed in vacuo to afford V as a white solid (0.595 g, 60%).

Preparation of analogs VII. Triazine V (200 mg, 0.63 mmol), boronic acid VI (150 mg, 0.72 mmol), and triphenylphosphine (25 mg, 0.10 mmol) were charged into a flask containing 4 mL of dioxane and 1.5 mL of 2 M Na₂CO₃ solution. After purging the mixture with Ar for 20 min, [Pd(PPh₃)₄] catalyst (36 mg, 0.03 mmol, 5 mol %) was added. The reaction mixture was heated at 100° C. for 4 h under an Ar atmosphere. After cooling the reaction mixture to rt, the solvent was removed under reduced pressure to produce a yellow oil. The residue was washed with water (20 mL) and extracted with CHCl₃ (20 mL). The organic layer was isolated and the remaining aqueous portion was washed again with CHCl₃ (3×20 mL). The organic extracts were combined, and the solvent was removed in vacuo. The crude product was purified by silica gel chromatography (ca. 120 mL) using CHCl₃-hexanes (3:1 v/v) as the eluent. If the compounds required further purification, they were triturated in hexanes in a sonicator for 5 min followed by re-crystallization from hexanes at −18° C. for 24 h.

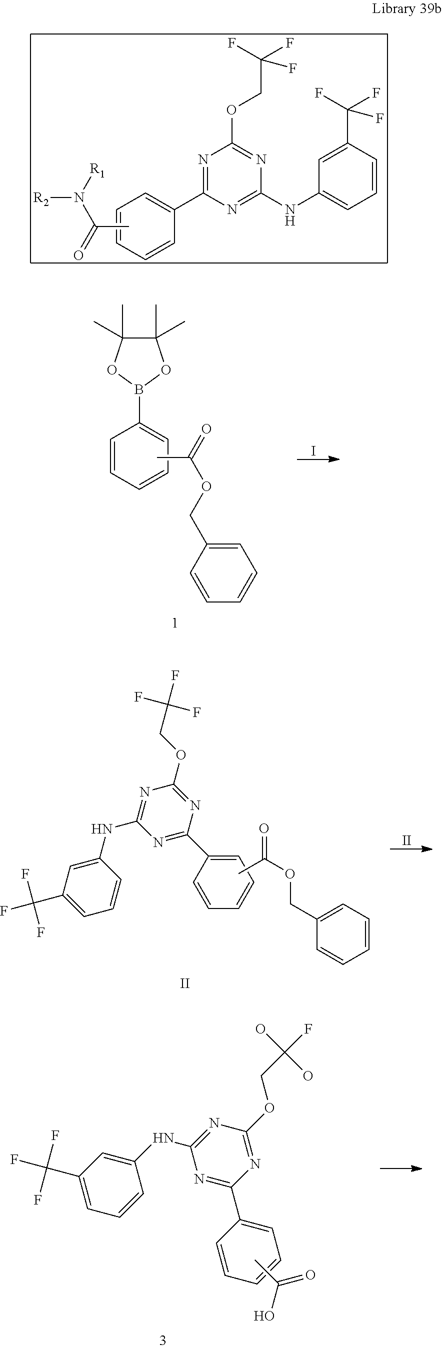

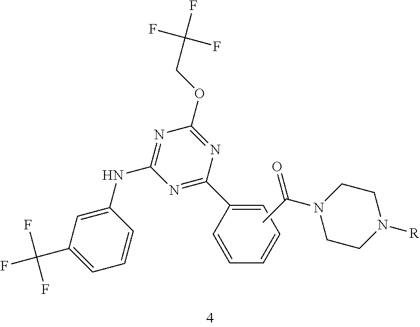

Intermediate 2 was synthesized according to the following procedure: Monochlorotriazine (10 g, 27 mmol), 1 (10.82 g, 32 mmol), and triphenylphosphine (1.05 g, 4 mmol) were charged into the flask containing 200 mL of dioxane and 30 mL of aqueous 2 M Na₂CO₃. After purging the mixture with Ar for 20 min, [Pd(PPh₃)₄] catalyst (5 mol %) was added. The reaction mixture was heated at 100° C. for 6 h under an Ar atmosphere. After cooling the reaction mixture to rt, the solvent was removed under reduced pressure to produce a yellow oil. The residue was washed with water (20 mL) and extracted with CHCl₃ (20 mL). The organic layer was isolated, and the remaining aqueous portion was washed again with CHCl₃ (3×20 mL). The organic extracts were combined, and the solvent was removed in vacuo. The crude product was purified by silica gel chromatography (ca. 120 mL) using CHCl₃-hexanes (3:1 v/v) as an eluent.

Intermediate 3 was synthesized according to the following procedure: To a stirred solution of 2 (2 mmol) in tetrahydrofuran (5 ml), ethanol (40 ml), and water (20 ml), was added 10% Pd/C (0.25 g). After stirring under hydrogen (1 atm) for 5 h, the mixture was filtered. The filtrate was concentrated in vacuo and the precipitate was re-crystallized from ether.

Intermediate 4 was synthesized according to the following procedure: Acid 3 (1 mmol) was dissolved in 2 ml of DMF and treated with CD (1 mmol). The reaction mixture was stirred for 1 hour at an ambient temperature, then treated with a solution of amine (1.2 mmol). This mixture was stirred at 70° C. for 8 hours, then cooled and concentrated under reduced pressure. The residue was washed with 10% aqueous NaHCO₃, then with water, and dried. The crude product was purified by silica gel chromatography using CH₂Cl₂-hexanes (3:1 v/v) as an eluent.

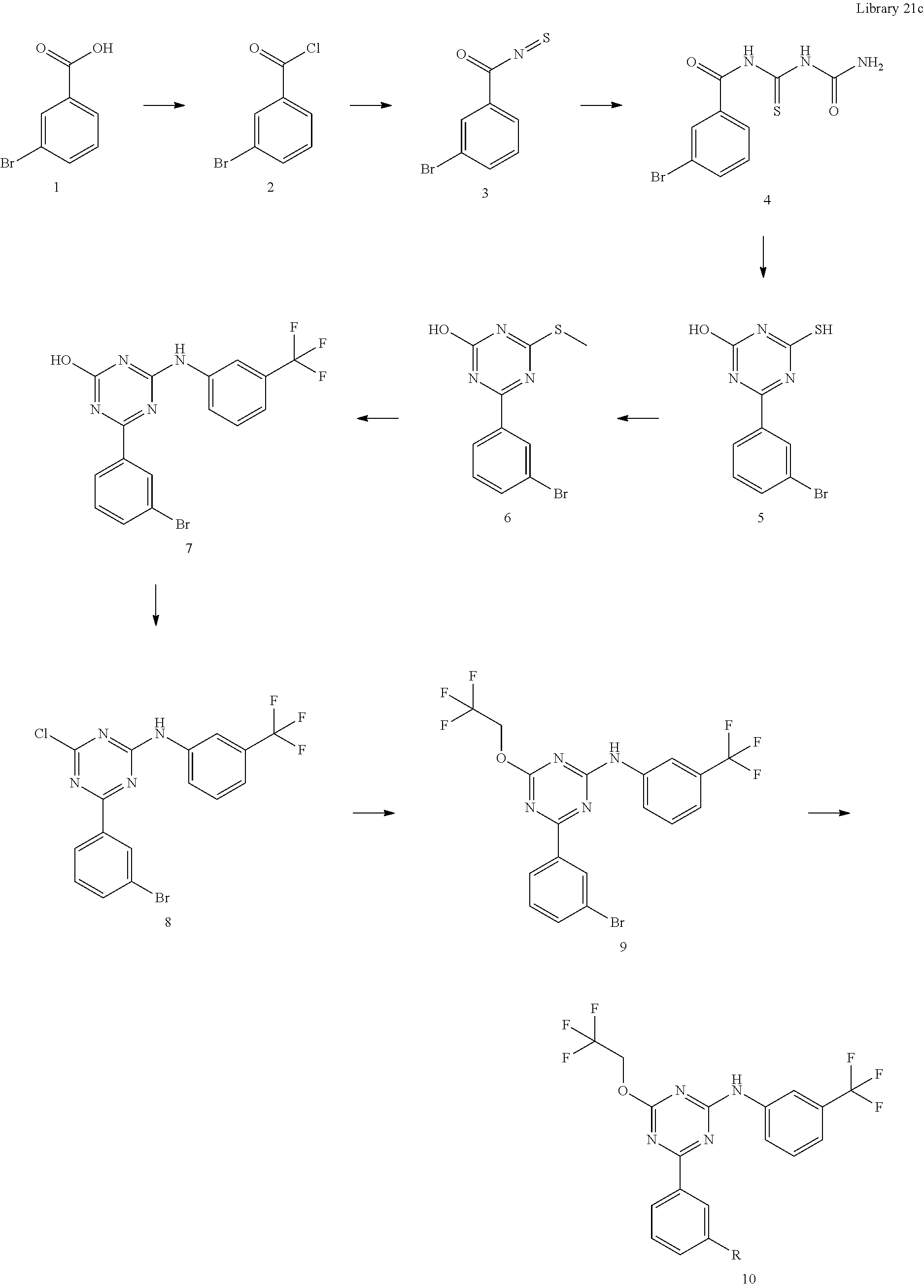

Preparation of 3 A solution of 2 (12 ml) in 10 ml of dry toluene was added to a suspension of dried KSCN (10.7 g) in 100 ml dry toluene. The reaction mixture was allowed to cool, KCl was filtered off and the filtrate evaporated leaving a residue, which solidifies on standing. This product was used in the next step without additional purification.

Preparation of 4 A solution of 3 in THF (50 ml) was added dropwise to a stirred suspension of urea (4.3 g) in THF (75 ml). The reaction mixture was stirred at reflux overnight. Then it was cooled and the precipitate was filtered, washed with ether, and dried.

Preparation of 5 Aqueous sodium hydroxide (50%, 6 g) was added to 10 g of 4 (33 mmol) and the reaction mixture was stirred for 90 min. The product was precipitated by the addition of glacial acetic acid, filtered, and washed with water. The solid was suspended in refluxing ethanol, filtered, and dried.

Preparation of 6 5 g of 5 (17 mmol) was suspended in methanol (100 mL), to which was added a solution of sodium methoxide (1.2 g) in methanol (30 mL). Methyl iodide (20 mmol) in methanol (10 mL) was added to the reaction mixture dropwise resulting in the formation of a precipitate. The solid was filtered, washed with water, air dried and re-crystallized from THF.

Preparation of 7 Intermediate 6 (14 mmol) was suspended in 42 mmol of 3-trifluoromethylaniline under Ar at 150° C. overnight. The reaction mixture was cooled, diluted with ether (200 mL) and filtered.

Preparation of 8 To the suspension of 7 (12 mmol) in chloroform (50 ml) was added 12 mmol of PCl₅ and 36 mmol of P° Cl₃ and the reaction mixture was refluxed overnight. At this time, the reaction mixture was diluted with toluene and the solvents were removed under reduced pressure. The product was used for the next step without additional purification.

Preparation of 9 To the suspension of potassium tert-butoxide (24 mmol) in 1,4-dioxane (50 ml) was added trifluoroethanol and the reaction mixture was stirred at room temperature for 1 hour. Then a solution of 8 (12 mmol) in 1,4-dioxane (100 ml) was added dropwise, and the reaction mixture was stirred at room temperature for 2 hours. Then reaction mixture was diluted with 1 L of water and extracted with ethyl acetate. The solvent was removed under reduced pressure and the precipitate was washed with hexane.

Preparation of 10 Bromide 10 (100 mg, 20 mmol) was dissolved in 1,4-dioxane (5 ml). Boronic acid (2.2 mmol), 10% aqueous solution of Na₂CO₃ (1 ml), and PdCl₂ (10 mol %) were added. The reaction mixture was stirred at 80° C. under Ar overnight. The reaction mixture was filtered and solvent was removed under reduced pressure to afford final compounds.

TABLE 39

| En | Structure | IUPAC Name | MW | Formula |
|---|---|---|---|---|
| 1 | 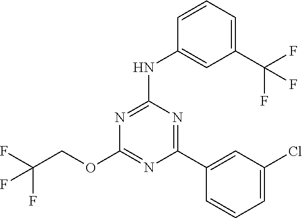 | 4-(3-chlorophenyl)-6-(2,2,2- trifluoroethoxy)-N-[3- (trifluoromethyl)phenyl]-1,3,5- triazin-2-amine | 448.8 | C18H11ClF6N4O |
| 2 | 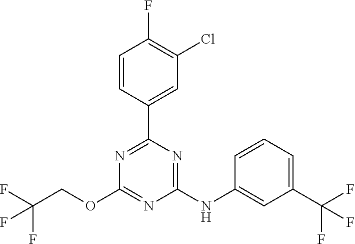 | 4-(3-chloro-4-fluorophenyl)-6-(2,2,2- trifluoroethoxy)-N-[3- (trifluoromethyl)phenyl]-1,3,5- triazin-2-amine | 466.8 | C18H10ClF7N4O |
| 3 | 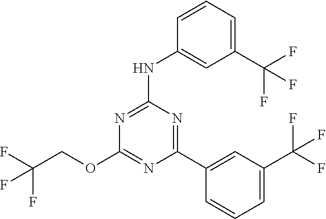 | 4-(2,2,2-trifluoroethoxy)-N,6-bis[3- (trifluoromethyl)phenyl]-1,3,5- triazin-2-amine | 482.3 | C19H11F9N4O |
| 4 | 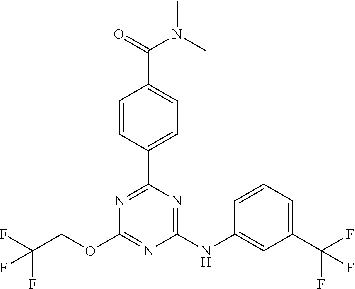 | N,N-dimethyl-4-[4-(2,2,2- trifluoroethoxy)-6-{[3- (trifluoromethyl)phenyl]amino}- 1,3,5-triazin-2-yl]benzamide | 485.4 | C21H17F6N5O2 |
| 5 | 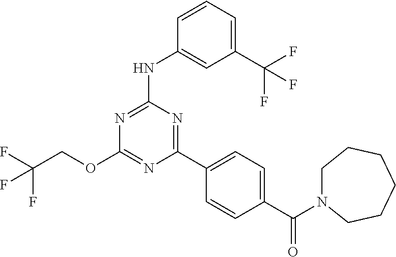 | azepan-1-yl{4-[4-(2,2,2- trifluoroethoxy)-6-{[3- (trifluoromethyl)phenyl]amino}- 1,3,5-triazin-2-yl]phenyl}methanone | 539.5 | C25H23F6N5O2 |
| 6 | 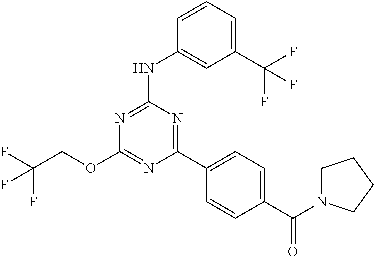 | pyrrolidin-1-yl{4-[4-(2,2,2- trifluoroethoxy)-6-{[3- (trifluoromethyl)phenyl]amino}- 1,3,5-triazin-2-yl]phenyl}methanone | 511.4 | C23H19F6N5O2 |
| 7 | 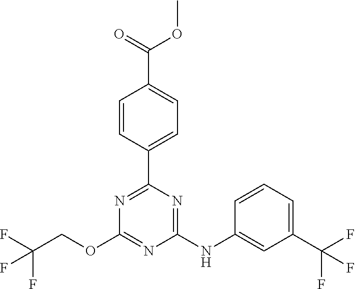 | methyl 4-[4-(2,2,2-trifluoroethoxy)-6- {[3-(trifluoromethyl)phenyl]amino}- 1,3,5-triazin-2-yl]benzoate | 472.3 | C20H14F6N4O3 |
| 8 | 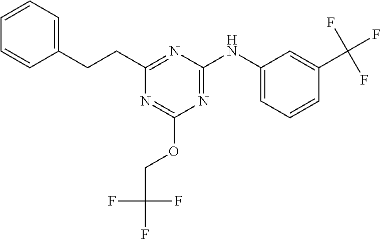 | 4-(2-phenylethyl)-6-(2,2,2- trifluoroethoxy)-N-[3- (trifluoromethyl)phenyl]-1,3,5- triazin-2-amine | 442.4 | C20H16F6N4O |
| 9 | 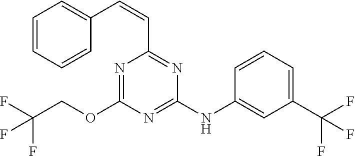 | 4-[(E)-2-phenylethenyl]-6-(2,2,2- trifluoroethoxy)-N-[3- (trifluoromethyl)phenyl]-1,3,5- triazin-2-amine | 440.3 | C20H14F6N4O |
| 10 | 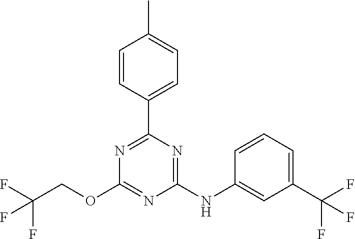 | 4-(4-methylphenyl)-6-(2,2,2- trifluoroethoxy)-N-[3- (trifluoromethyl)phenyl]-1,3,5- triazin-2-amine | 428.3 | C19H14F6N4O |
| 11 | 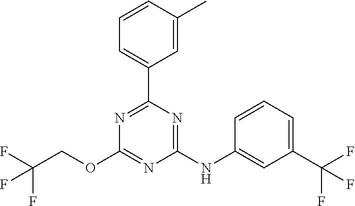 | 4-(3-methylphenyl)-6-(2,2,2- trifluoroethoxy)-N-[3- (trifluoromethyl)phenyl]-1,3,5- triazin-2-amine | 428.3 | C19H14F6N4O |
| 12 | 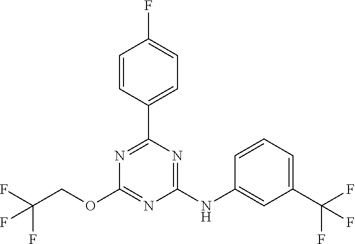 | 4-(4-fluorophenyl)-6-(2,2,2- trifluoroethoxy)-N-[3- (trifluoromethyl)phenyl]-1,3,5- triazin-2-amine | 432.3 | C18H11F7N4O |
| 13 | 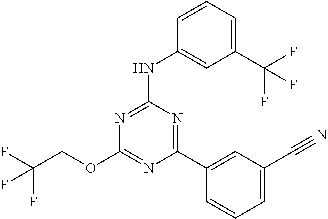 | 3-[4-(2,2,2-trifluoroethoxy)-6-{[3- (trifluoromethyl)phenyl]amino}- 1,3,5-triazin-2-yl]benzonitrile | 439.3 | C19H11F6N5O |
| 14 | 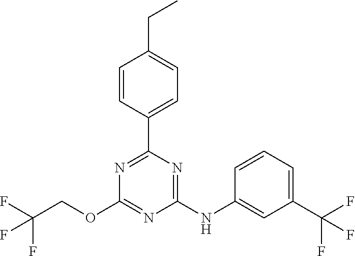 | 4-(4-ethylphenyl)-6-(2,2,2- trifluoroethoxy)-N-[3- (trifluoromethyl)phenyl]-1,3,5- triazin-2-amine | 442.4 | C20H16F6N4O |
| 15 | 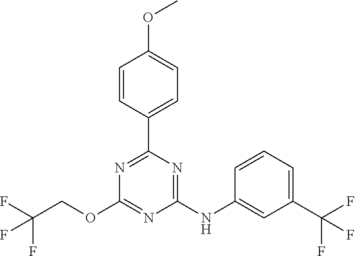 | 4-(4-methoxyphenyl)-6-(2,2,2- trifluoroethoxy)-N-[3- (trifluoromethyl)phenyl]-1,3,5- triazin-2-amine | 444.3 | C19H14F6N4O2 |
| 16 | 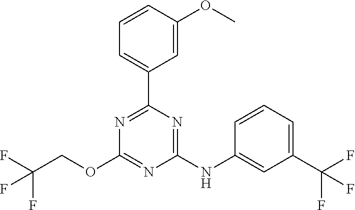 | 4-(3-methoxyphenyl)-6-(2,2,2- trifluoroethoxy)-N-[3- (trifluoromethyl)phenyl]-1,3,5- triazin-2-amine | 444.3 | C19H14F6N4O2 |
| 17 | 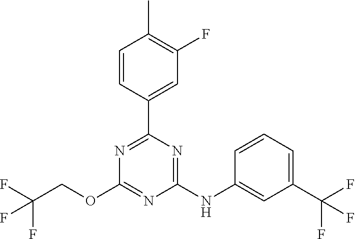 | 4-(3-fluoro-4-methylphenyl)-6- (2,2,2-trifluoroethoxy)-N-[3- (trifluoromethyl)phenyl]-1,3,5- triazin-2-amine | 446.3 | C19H13F7N4O |
| 18 | 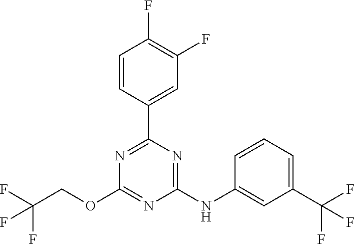 | 4-(3,4-difluorophenyl)-6-(2,2,2- trifluoroethoxy)-N-[3- (trifluoromethyl)phenyl]-1,3,5- triazin-2-amine | 450.3 | C18H10F8N4O |
| 19 | 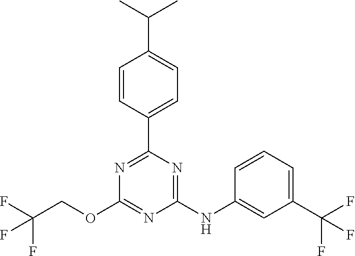 | 4-[4-(propan-2-yl)phenyl]-6-(2,2,2- trifluoroethoxy)-N-[3- (trifluoromethyl)phenyl]-1,3,5- triazin-2-amine | 456.4 | C21H18F6N4O |
| 20 | 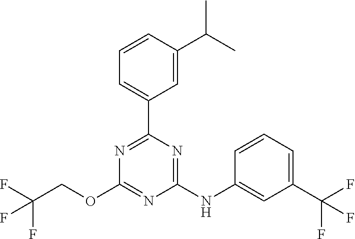 | 4-[3-(propan-2-yl)phenyl]-6-(2,2,2- trifluoroethoxy)-N-[3- (trifluoromethyl)phenyl]-1,3,5- triazin-2-amine | 456.4 | C21H18F6N4O |
| 21 | 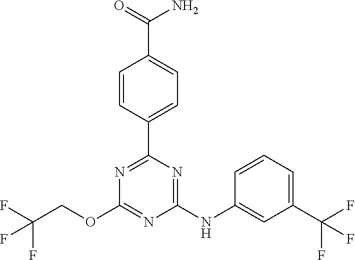 | 4-[4-(2,2,2-trifluoroethoxy)-6-{[3- (trifluoromethyl)phenyl]amino}- 1,3,5-triazin-2-yl]benzamide | 457.3 | C19H13F6N5O2 |
| 22 | 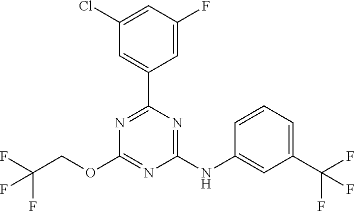 | 4-(3-chloro-5-fluorophenyl)-6-(2,2,2- trifluoroethoxy)-N-[3- (trifluoromethyl)phenyl]-1,3,5- triazin-2-amine | 466.8 | C18H10ClF7N4O |
| 23 | 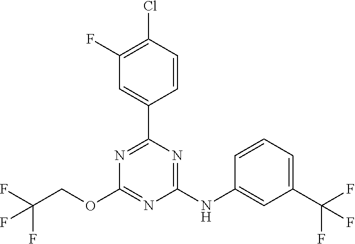 | 4-(4-chloro-3-fluorophenyl)-6-(2,2,2- trifluoroethoxy)-N-[3- (trifluoromethyl)phenyl]-1,3,5- triazin-2-amine | 466.8 | C18H10ClF7N4O |
| 24 | 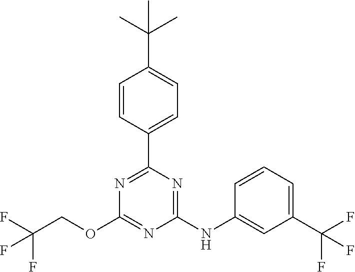 | 4-(4-tert-butylphenyl)-6-(2,2,2- trifluoroethoxy)-N-[3- (trifluoromethyl)phenyl]-1,3,5- triazin-2-amine | 470.4 | C22H20F6N4O |
| 25 | 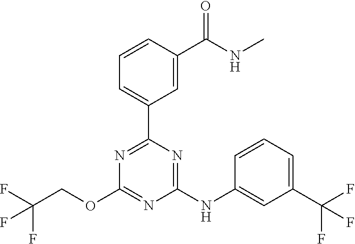 | N-methyl-3-[4-(2,2,2- trifluoroethoxy)-6-{[3- (trifluoromethyl)phenyl]amino}- 1,3,5-triazin-2-yl]benzamide | 471.4 | C20H15F6N5O2 |
| 26 | 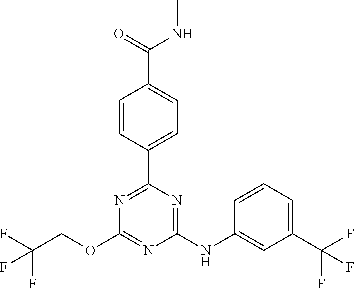 | N-methyl-4-[4-(2,2,2- trifluoroethoxy)-6-{[3- (trifluoromethyl)phenyl]amino}- 1,3,5-triazin-2-yl]benzamide | 471.4 | C20H15F6N5O2 |
| 27 | 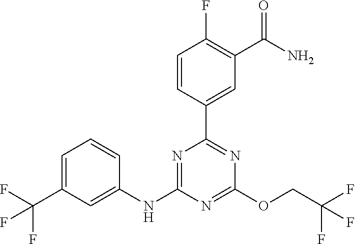 | 2-fluoro-5-[4-(2,2,2-trifluoroethoxy)- 6-{[3-(trifluoromethyl)phenyl]amino}- 1,3,5-triazin-2-yl]benzamide | 475.3 | C19H12F7N5O2 |
| 28 | 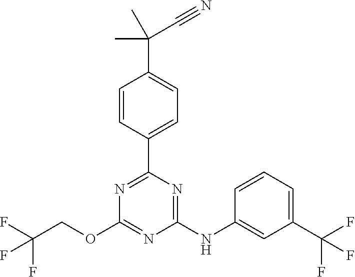 | 2-methyl-2-{4-[4-(2,2,2- trifluoroethoxy)-6-{[3- (trifluoromethyl)phenyl]amino}- 1,3,5-triazin-2- yl]phenyl}propanenitrile | 481.4 | C22H17F6N5O |
| 29 | 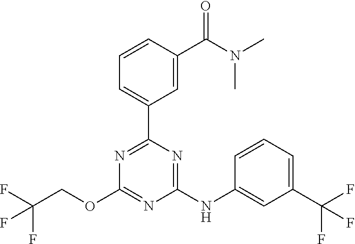 | N,N-dimethyl-3-[4-(2,2,2- trifluoroethoxy)-6-{[3- (trifluoromethyl)phenyl]amino}- 1,3,5-triazin-2-yl]benzamide | 485.4 | C21H17F6N5O2 |
| 30 | 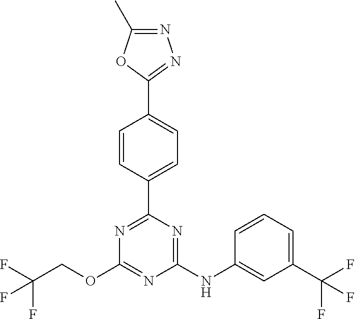 | 4-[4-(5-methyl-1,3,4-oxadiazol-2- yl)phenyl]-6-(2,2,2-trifluoroethoxy)- N-[3-(trifluoromethyl)phenyl]-1,3,5- triazin-2-amine | 496.4 | C21H14F6N6O2 |
| 31 | 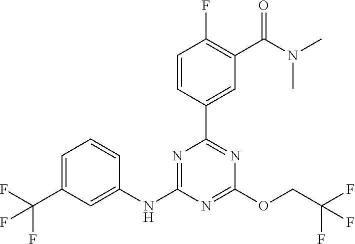 | 2-fluoro-N,N-dimethyl-5-[4-(2,2,2- trifluoroethoxy)-6-{[3- (trifluoromethyl)phenyl]amino}- 1,3,5-triazin-2-yl]benzamide | 503.4 | C21H16F7N5O2 |
| 32 | 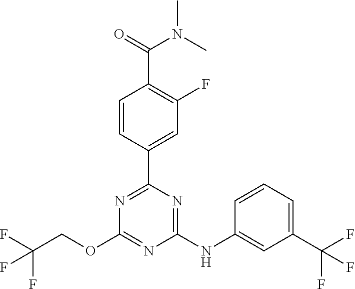 | 2-fluoro-N,N-dimethyl-4-[4-(2,2,2- trifluoroethoxy)-6-{[3- (trifluoromethyl)phenyl]amino}- 1,3,5-triazin-2-yl]benzamide | 503.4 | C21H16F7N5O2 |
| 33 | 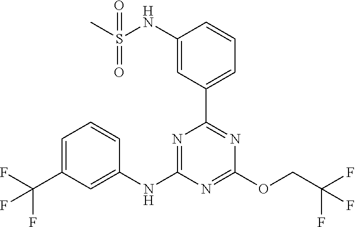 | N-{3-[4-(2,2,2-trifluoroethoxy)-6-{[3- (trifluoromethyl)phenyl]amino}- 1,3,5-triazin-2- yl]phenyl}methanesulfonamide | 508.4 | C19H15F6N5O3S |
| 34 | 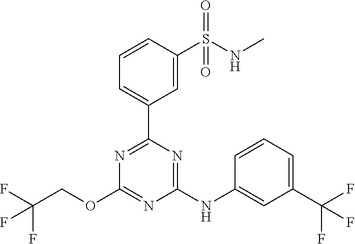 | N-methyl-3-[4-(2,2,2- trifluoroethoxy)-6-{[3- (trifluoromethyl)phenyl]amino}- 1,3,5-triazin-2- yl]benzenesulfonamide | 508.4 | C19H15F6N5O3S |
| 35 | 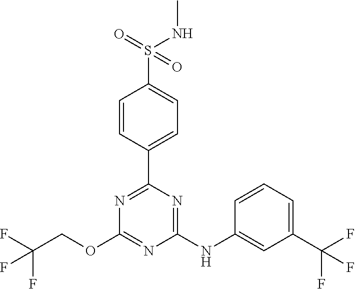 | N-methyl-4-[4-(2,2,2- trifluoroethoxy)-6-{[3- (trifluoromethyl)phenyl]amino}- 1,3,5-triazin-2- yl]benzenesulfonamide | 508.4 | C19H15F6N5O3S |
| 36 | 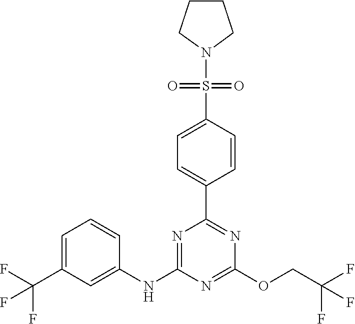 | 4-[4-(pyrrolidin-1-ylsulfonyl)phenyl]- 6-(2,2,2-trifluoroethoxy)-N-[3- (trifluoromethyl)phenyl]-1,3,5- triazin-2-amine | 548.5 | C22H19F6N5O3S |
| 37 | 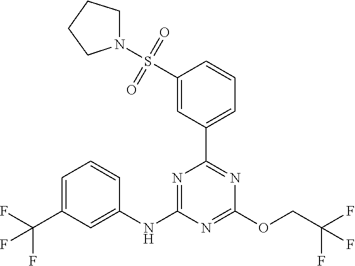 | 4-[3-(pyrrolidin-1-ylsulfonyl)phenyl]- 6-(2,2,2-trifluoroethoxy)-N-[3- (trifluoromethyl)phenyl]-1,3,5- triazin-2-amine | 548.5 | C22H19F6N5O3S |
| 38 | 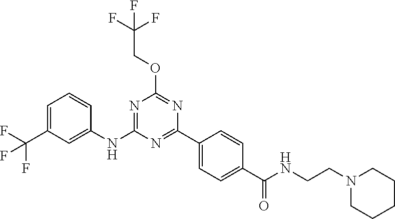 | N-[2-(piperdin-1-yl)ethyl]-4-[4- (2,2,2-trifluoroethoxy)-6-{[3- (trifluoromethyl)phenyl]amino}- 1,3,5-triazin-2-yl]benzamide | 568.5 | C26H26F6N6O2 |
| 39 | 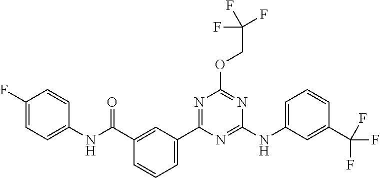 | N-(4-fluorophenyl)-3-(4-(2,2,2- trifluoroethoxy)-6-{[3- (trifluoromethyl)phenyl]amino}- 1,3,5-triazin-2-yl)benzamide | 551.4 | C25H16F7N5O2 |
| 40 | 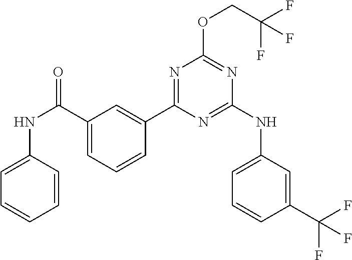 | N-phenyl-3-(4-(2,2,2- trifluoroethoxy)-6-{[3- (trifluoromethyl)phenyl]amino}- 1,3,5-triazin-2-yl)benzamide | 533.4 | C25H17F6N5O2 |
| 41 | 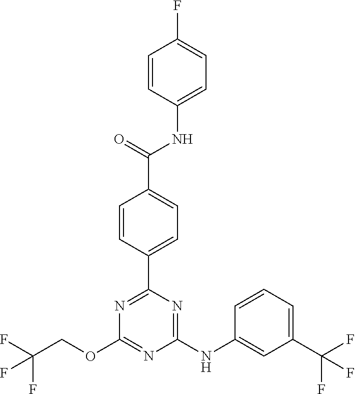 | N-(4-fluorophenyl)-4-[4-(2,2,2- trifluoroethoxy)-6-{[3- (trifluoromethyl)phenyl]amino}- 1,3,5-triazin-2-yl]benzamide | 551.4 | C25H16F7N5O2 |
| 42 | 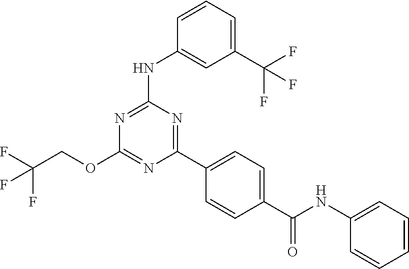 | N-phenyl-4-[4-(2,2,2- trifluoroethoxy)-6-{[3- (trifluoromethyl)phenyl]amino}- 1,3,5-triazin-2-yl]benzamide | 533.4 | C25H17F6N5O2 |
| 43 | 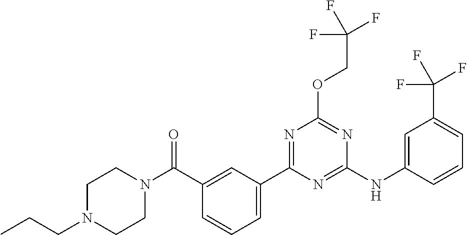 | 4-{3-[(4-propylpiperazin-1- yl)carbonyl]phenyl}-6-(2,2,2- trifluoroethoxy)-N-[3- (trifluoromethyl)phenyl]-1,3,5- triazin-2-amine | 568.5 | C26H26F6N6O2 |
| 44 | 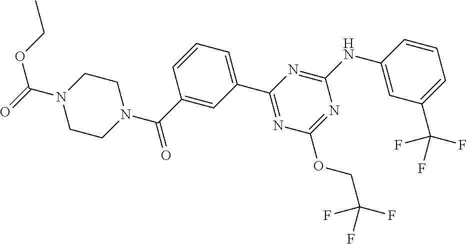 | ethyl 4-[3-(4-(2,2,2-trifluoroethoxy)- 6-{[3-(trifluoromethyl)phenyl]amino}- 1,3,5-triazin-2- yl)benzoyl]piperazine-1-carboxylate | 598.5 | C26H24F6N6O4 |
| 45 | 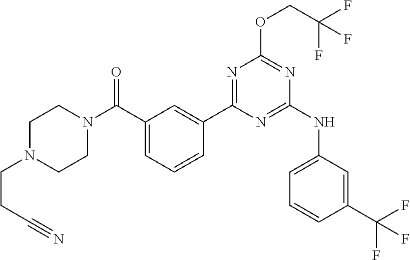 | 3-{4-[3-(4-(2,2,2-trifluoroethoxy)-6- {[3-(trifluoromethyl)phenyl]amino}- 1,3,5-triazin-2-yl)benzoyl]piperazin- 1-yl}propanenitrile | 579.5 | C26H23F6N7O2 |
| 46 | 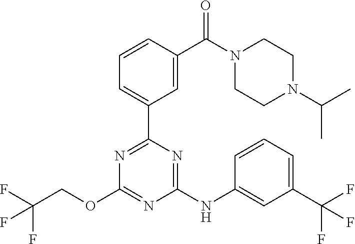 | [4-(propan-2-yl)piperazin-1-yl]{3-[4- (2,2,2-trifluoroethoxy)-6-{[3- (trifluoromethyl)phenyl]-amino}- 1,3,5-triazin-2-yl]phenyl}methanone | 568.5 | C26H26F6N6O2 |
| 47 | 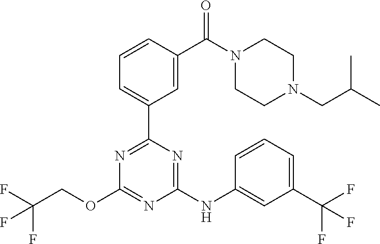 | [4-(2-methylpropyl)piperazin-1-yl]{3- [4-(2,2,2-trifluoroethoxy)-6-{[3- (trifluoromethyl)phenyl]-amino}- 1,3,5-triazin-2-yl]phenyl}methanone | 582.5 | C27H28F6N6O2 |
| 48 | 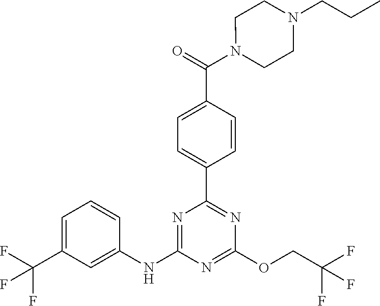 | (4-propylpiperazin-1-yl){4-[4-(2,2,2- trifluoroethoxy)-6-{[3- (trifluoromethyl)phenyl]amino}- 1,3,5-triazin-2-yl]phenyl}methanone | 568.5 | C26H26F6N6O2 |
| 49 | 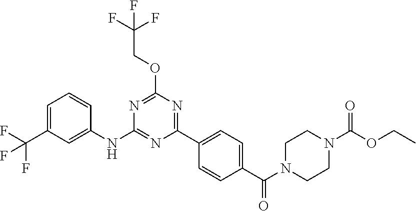 | ethyl 4-({4-[4-(2,2,2-trifluoroethoxy)- 6-{[3-(trifluoromethyl)phenyl]amino}- 1,3,5-triazin-2- yl]phenyl}carbonyl)piperazine-1- carboxylate | 598.5 | C26H24F6N6O4 |
| 50 | 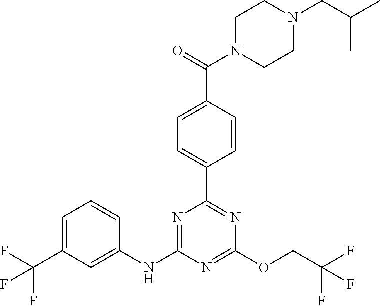 | [4-(2-methylpropyl)piperazin-1-yl]{4- [4-(2,2,2-trifluoroethoxy)-6-{[3- (trifluoromethyl)phenyl]-amino}- 1,3,5-triazin-2-yl]phenyl}methanone | 582.5 | C27H28F6N6O2 |
| 51 | 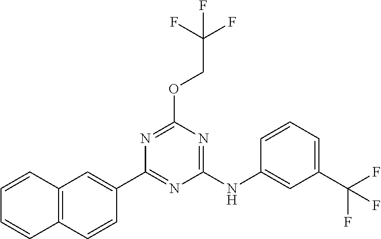 | 4-(naphthalen-2-yl)-6-(2,2,2- trifluoroethoxy)-N-[3- (trifluoromethyl)phenyl]-1,3,5- triazin-2-amine | 464.4 | C22H14F6N4O |
| 52 | 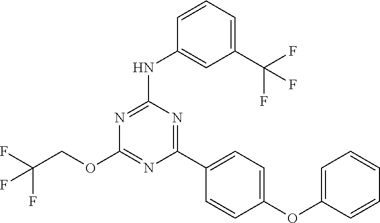 | 4-(4-phenoxyphenyl)-6-(2,2,2- trifluoroethoxy)-N-(3- (trifluoromethyl)phenyl)-1,3,5- triazin-2-amine | 506.4 | C24H16F6N4O2 |
| 53 | 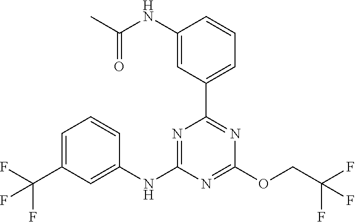 | N-{3-[4-(2,2,2-trifluoroethoxy)-6-{[3- (trifluoromethyl)phenyl]amino}- 1,3,5-triazin-2-yl]phenyl}acetamide | 471.4 | C20H15F6N5O2 |
| 54 | 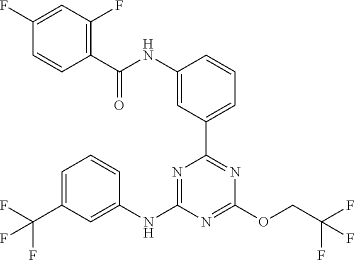 | 2,4-difluoro-N-{3-[4-(2,2,2- trifluoroethoxy)-6-{[3- (trifluoromethyl)phenyl]amino}- 1,3,5-triazin-2-yl]phenyl}benzamide | 569.4 | C25H15F8N5O2 |
| 55 | 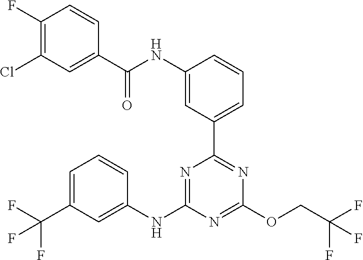 | 3-chloro-4-fluoro-N-{3-[4-(2,2,2- trifluoroethoxy)-6-{[3- (trifluoromethyl)phenyl]amino}- 1,3,5-triazin-2-yl]phenyl}benzamide | 585.9 | C25H15ClF7N5O2 |
| 56 | 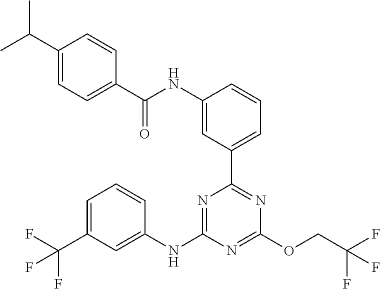 | 4-(propan-2-yl)-N-{3-[4-(2,2,2- trifluoroethoxy)-6-{[3- (trifluoromethyl)phenyl]amino}- 1,3,5-triazin-2-yl]phenyl}benzamide | 575.5 | C28H23F6N5O2 |
| 57 | 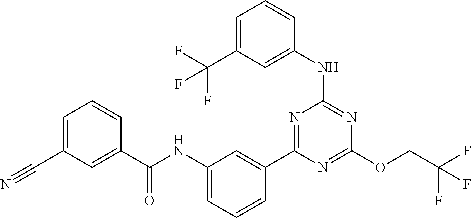 | 3-cyano-N-{3-[4-(2,2,2- trifluoroethoxy)-6-{[3- (trifluoromethyl)phenyl]amino}- 1,3,5-triazin-2-yl]phenyl}benzamide | 558.4 | C26H16F6N6O2 |
| 58 | 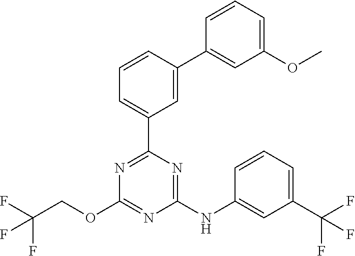 | 4-(3″-methoxybiphenyl-3-yl)-6- (2,2,2-trifluoroethoxy)-N-[3- (trifluoromethyl)phenyl]-1,3,5- triazin-2-amine | 520.4 | C25H18F6N4O2 |
| 59 | 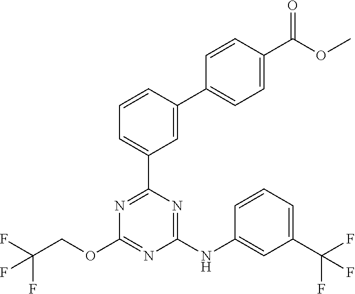 | methyl 3′-(4-(2,2,2-trifluoroethoxy)- 6-(3-(trifluoromethyl)phenylamino)- 1,3,5-triazin-2-yl)biphenyl-4- carboxylate | 548.4 | C26H18F6N4O3 |
| 60 | 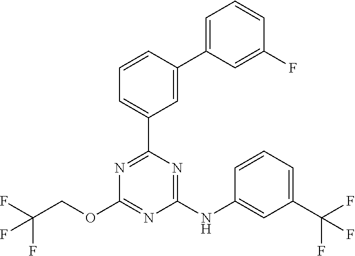 | 4-(3′-fluorobiphenyl-3-yl)-6-(2,2,2- trifluoroethoxy)-N-(3- (trifluoromethyl)phenyl)-1,3,5- triazin-2-amine | 508.4 | C24H15F7N4O |
| 61 | 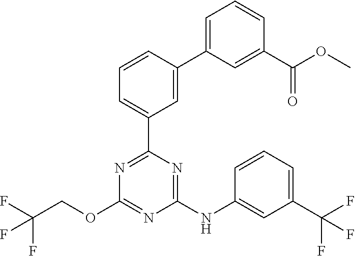 | 4-(3′-fluorobiphenyl-3-yl)-6-(2,2,2- trifluoroethoxy)-N-(3- (trifluoromethyl)phenyl)-1,3,5- triazin-2-amine | 548.4 | C26H18F6N4O3 |

### Procedures and Analytical Data for Table 39.

Entries 1 to 7, and 10 to 38, 51 and 52 were prepared according to the procedures for Library 39a. Entries 39 to 50 and 53 to 57 were prepared by the methods described for Library 39b. Entries 58 to 61 were prepared by the procedures for Library 39c.

### 1. 4-(3-chlorophenyl)-6-(2,2,2-trifluoroethoxy)-N-[3-(trifluoromethyl)phenyl]-1,3,5-triazin-2-amine

LCMS: M+1=449.3; NMR 1H, DMSO-d6 δ, ppm: 5.13 m (2H); 7.45 d (1H); 7.56 t (1H); 7.62 s (1H); 7.66 t (1H); 7.94 d (1H); 8.31 d (1H); 8.36 d (1H); 8.38 s (1H); 10.40 s (1H).

### 2. 4-(3-chloro-4-fluorophenyl)-6-(2,2,2-trifluoroethoxy)-N-[3-(trifluoromethyl)phenyl]-1,3,5-triazin-2-amine

LCMS: M+1=467.1 NMR 1H, DMSO-d6 δ, ppm: 5.15 m (2H); 7.45 d (1H); 7.52-7.68 m (2H); 7.94 d (1H); 8.28 s (1H); 8.34-8.52 m (2H); 10.45 s (1H).

### 3. 4-(2,2,2-trifluoroethoxy)-N,6-bis[3-(trifluoromethyl)phenyl]-1,3,5-triazin-2-amine

LCMS: M+1=483.3; 1H NMR, DMSO-d6 δ, ppm: 5.15 m (2H); 7.46 d (1H); 7.62 t (1H); 7.82 t (1H); 7.94 d (1H); 7.98 d (1H); 8.33 s (1H); 8.66 s (1H); 8.68 d (1H); 10.50 s 1H).

### 4. N,N-dimethyl-4-[4-(2,2,2-trifluoroethoxy)-6-{[3-(trifluoromethyl)phenyl]amino}-1,3,5-triazin-2-yl]benzamide

LCMS: M+1=485.3; 1H NMR, DMSO-d6 δ, ppm: 3.0 (6H, m); 5.1 (2H, q); 7.45 (1H, d); 7.6 (3H, m); 8.0 (1H, d); 8.25 (1H, s); 8.45 (2H, d); 10.4 (1H, s).

### 5. azepan-1-yl{4-[4-(2,2,2-trifluoroethoxy)-6-{[3-(trifluoromethyl)phenyl]amino}-1,3,5-triazin-2-yl]phenyl}methanone

LCMS: M+1=539.4; 1H NMR, DMSO-d6 δ, ppm: 1.6 (8H, m); 3.4 (4H, s); 5.15 (2H, q); 7.55 (4H, m); 8.0 (1H, d); 8.25 (1H, s); 8.45 (2H, d); 10.4 (1H, s).

### 6. pyrrolidin-1-yl{4-[4-(2,2,2-trifluoroethoxy)-6-{[3-(trifluoromethyl)phenyl]amino}-1,3,5-triazin-2-yl]phenyl}methanone

LCMS: M+1=511.4; 1H NMR, DMSO-d6 δ, ppm: 1.9 (4H, s); 3.5 (4H, s); 5.1 (2H, q); 7.4 (1H, d); 7.6 (3H, m); 8.0 (1H, d); 8.3 (1H, s); 8.45 (2H, d); 10.45 (1H, s).

### 7. methyl 4-[4-(2,2,2-trifluoroethoxy)-6-{[3-(trifluoromethyl)phenyl]amino}-1,3,5-triazin-2-yl]benzoate

LCMS: M+1=472.3; 1H NMR, DMSO-d6 δ, ppm: 3.9 (3H, s); 5.1 (2H, q); 7.45 (1H, d); 7.6 (1H, t); 7.95 (1H, d); 8.15 (2H, d); 8.25 (1H, s); 8.5 (2H, d); 10.5 (1H, s).

### 8. 4-(2-phenylethyl)-6-(2,2,2-trifluoroethoxy)-N-[3-(trifluoromethypphenyl]-1,3,5-triazin-2-amine

LCMS: M+1=443; 1H NMR, CDCl3, δ, ppm: 3.22 m (4H, CH); 4.6 m (4H CH); 7.22 m (6H, CH); 7.4 d (1H, CH); 7.5 t (1H, CH); 7.66 s (1H; CH;); 8.2 s (1H, NH).

### 9. 4-[(E)-2-phenylethenyl]-6-(2,2,2-trifluoroethoxy)-N-[3-(trifluoromethyl)phenyl]-1,3,5-triazin-2-amine

LCMS: M+1=441; 1H NMR, 00013-d6 δ, ppm: 4.89 m (2H); 6.95 d (1H); 7.45 m (6H); 7.68 m (3H); 8.18 bs (1H); 8.21 bs (1H)

### 10. 4-(4-methylphenyl)-6-(2,2,2-trifluoroethoxy)-N-[3-(trifluoromethyl)phenyl]-1,3,5-triazin-2-amine

LCMS: M+1=429.4; 1H NMR, DMSO-d6 δ, ppm: 2.40 s (3H); 5.15 m (2H); 7.34-7.46 m (3H); 7.61 t (1H); 7.70 d (1H); 8.31 m (3H); 10.30 s (1H).

### 11. 4-(3-methylphenyl)-6-(2,2,2-trifluoroethoxy)-N-[3-(trifluoromethyl)phenyl]-1,3,5-triazin-2-amine

LCMS: M+1=429.3; 1H NMR, DMSO-d6 δ, ppm: 2.40 s (3H); 5.15 m (2H); 7.40-7.48 m (3H); 7.61 t (1H); 7.94 d (1H); 8.22 m (2H); 8.37 s (1H); 10.35 s (1H).

### 12. 4-(4-fluorophenyl)-6-(2,2,2-trifluoroethoxy)-N-[3-(trifluoromethyl)phenyl]-1,3,5-triazin-2-amine

LCMS: M+1=433.5; 1H NMR, DMSO-d6 δ, ppm: 5.12 m (2H); 7.35 d (1H); 7.37 d (1H); 7.62 t (1H); 7.99 d (1H); 8.25 s (1H); 8.44 d (1H); 8.29 s (1H); 10.30 s (1H).

### 13. 3-[4-(2,2,2-trifluoroethoxy)-6-{[3-(trifluoromethyl)phenyl]amino}-1,3,5-triazin-2-yl]benzonitrile

LCMS: M+1=440.5; 1H NMR, DMSO-d6 δ, ppm: 5.15 m (2H); 7.46 d (1H); 7.63 t (1H); 7.78 t (1H); 7.95 d (1H); 8.05 d (1H); 8.28 s (1H); 8.66 m (2H); 10.45 s (1H).

### 14. 4-(4-ethylphenyl)-6-(2,2,2-trifluoroethoxy)-N-[3-(trifluoromethyl)phenyl]-1,3,5-triazin-2-amine

LCMS: M+1=443.7; 1H NMR, DMSO-d6 δ, ppm: 1.25 t (3H); 2.75 m (2H); 5.15 m (2H); 7.40 m (3H); 7.61 t (1H); 7.98 d (1H); 8.32 m (3H); 10.3 s (1H).

### 15. 4-(4-methoxyphenyl)-6-(2,2,2-trifluoroethoxy)-N-[3-(trifluoromethyl)phenyl]-1,3,5-triazin-2-amine

LCMS: M+1=445.6; 1H NMR, DMSO-d6 δ, ppm: 3.80 (3H); 5.15 m (2H); 7.08 d (2H); 7.42 d (1H); 7.60 t (1H); 8.00 d (1H); 8.26-8.42 m (3H); 10.25 s (1H).

### 16. 4-(3-methoxyphenyl)-6-(2,2,2-trifluoroethoxy)-N-[3-(trifluoromethyl)phenyl]-1,3,5-triazin-2-amine

LCMS: M+1=445.7; 1H NMR, DMSO-d6 δ, ppm: 3.90 s (3H); 5.15 m (2H); 7.21 d (1H); 7.44 d (1H); 7.47 t (1H); 7.62 t (1H); 7.74 d (1H); 7.79 s (1H); 8.01 d (1H); 8.29 s (1H); 10.30 s (1H).

### 17. 4-(3-fluoro-4-methylphenyl)-6-(2,2,2-trifluoroethoxy)-N-[3-(trifluoromethyl)phenyl]-1,3,5-triazin-2-amine

LCMS: M+1=447.3; 1H NMR, DMSO-d6 δ, ppm: 2.34 s (3H); 5.13 m (2H); 7.44 d (1H); 7.48 s (1H); 7.61 t (1H); 7.95 d (1H); 8.04 d (1H); 8.13 d (1H); 8.29 s (1H); 10.35 s (1H).

### 18. 4-(3,4-difluorophenyl)-6-(2,2,2-trifluoroethoxy)-N-[3-(trifluoromethyl)phenyl]-1,3,5-triazin-2-amine

LCMS: M+1=451.3; 1H NMR, DMSO-d6 δ, ppm: 5.15 m (2H); 7.43 d (1H); 7.60 m (2H); 7.96 d (1H); 8.22-8.32 m (3H); 10.40 s (1H).

### 19. 4-[4-(propan-2-yl)phenyl]-6-(2,2,2-trifluoroethoxy)-N-[3-(trifluoromethyl)phenyl]-1,3,5-triazin-2-amine

LCMS: M+1=457.5; 1H NMR, DMSO-d6 δ, ppm: 1.28 s (3H); 1.32 s (3H); 3.05 m (1H); 5.12 m (2H); 7.38-7.44 m (3H); 7.61 t (1H); 7.99 d (1H); 8.27-8.38 m (3H); 10.15 s (1H).

### 20. 4-[3-(propan-2-yl)phenyl]-6-(2,2,2-trifluoroethoxy)-N-[3-(trifluoromethyl)phenyl]-1,3,5-triazin-2-amine

LCMS: M+1=457.6; 1H NMR, DMSO-d6 δ, ppm: 1.3 m (6H); 3.05 m (1H); 5.15 m (2H); 7.4-7.68 m (4H); 7.96 d (1H); 8.22 d (1H); 8.28 s (1H); 8.36 s (1H); 10.35 s (1H).

### 21. 4-[4-(2,2,2-trifluoroethoxy)-6-{[3-(trifluoromethyl)phenyl]amino}-1,3,5-triazin-2-yl]benzamide

LCMS: M+1=458.4; 1H NMR, DMSO-d6 δ, ppm: 5.17 m (2H); 7.45 d (2H); 7.63 t (3H); 8.00 m (3H); 8.32 s (1H); 8.45 d (2H); 1025 s (1H).

### 22. 4-(3-chloro-5-fluorophenyl)-6-(2,2,2-trifluoroethoxy)-N-[3-(trifluoromethyl)phenyl]-1,3,5-triazin-2-amine

LCMS: M+1=467.4; 1H NMR, DMSO-d6 δ, ppm: 5.15 m (2H); 7.45 d (1H); 7.55-7.67 m (2H); 7.92 d (1H); 8.05 s (1H); 8.22 s (1H); 8.28 s (1H); 10.40 s (1H).

### 23. 4-(4-chloro-3-fluorophenyl)-6-(2,2,2-trifluoroethoxy)-N-[3-(trifluoromethyl)phenyl]-1,3,5-triazin-2-amine

LCMS: M+1=467.5; 1H NMR, DMSO-d6 δ, ppm: 5.15 m (2H); 7.45 d (1H); 7.62 t (1H); 7.76 t (1H); 7.97 d (3H); 8.18-8.28 m (3H); 10.45 s (1H).

### 24. 4-(4-tert-butylphenyl)-6-(2,2,2-trifluoroethoxy)-N-[3-(trifluoromethyl)phenyl]-1,3,5-triazin-2-amine

LCMS: M+1=471.5; 1H NMR, DMSO-d6 δ, ppm: 1.35 s (9H); 5.12 m (2H); 7.43 d (1H); 7.52-7.64 m (3H); 8.00 d (1H); 8.24-8.37 m (3H); 10.20 s (1H).

### 25. N-methyl-3-[4-(2,2,2-trifluoroethoxy)-6-{[3-(trifluoromethyl)phenyl]amino}-1,3,5-triazin-2-yl]benzamide

LCMS: M+1=472.7; 1H NMR, DMSO-d6 δ, ppm: 2.85 m (3H); 5.15 m (2H); 7.45 d (1H); 7.63 m (2H); 8.05 m (2H); 8.30 s (1H); 8.50 d (1H); 8.85 s (1H); 10.50 s (1H).

### 26. N-methyl-4-[4-(2,2,2-trifluoroethoxy)-6-{[3-(trifluoromethyl)phenyl]amino}-1,3,5-triazin-2-yl]benzamide

LCMS: M+1=472.7; 1H NMR, DMSO-d6 δ, ppm: 2.85 d (3H); 5.15 m (2H); 7.45 d (1H); 7.62 t (1H); 7.93-8.02 m (3H); 8.24-8.48 m (4H); 10.45 s (1H).

### 27. 2-fluoro-5-[4-(2,2,2-trifluoroethoxy)-6-{[3-(trifluoromethyl)phenyl]amino}-1,3,5-triazin-2-yl]benzamide

LCMS: M+1=476.3; 1H NMR, DMSO-d6 δ, ppm: 5.20 m (2H); 7.44-7.57 m (2H); 7.64 t (1H); 7.78 s (1H); 7.88 s (1H); 7.97 d (1H); 8.30 s (1H); 8.52 d (1H); 8.72 s (1H); 10.80 s (1H).

### 28. 2-methyl-2-{4-[4-(2,2,2-trifluoroethoxy)-6-{[3-(trifluoromethyl)phenyl]amino}-1,3,5-triazin-2-yl]phenyl}propanenitrile

LCMS: M+1=482.3; 1H NMR, DMSO-d6 δ, ppm: 1.80 s (6H); 5.15 m (2H); 7.44 d (1H); 7.62 t (1H); 7.72 d (2H); 8.00 d (1H); 8.28 s (1H); 8.44 d (2H); 10.40 s (1H).

### 29. N,N-dimethyl-3-[4-(2,2,2-trifluoroethoxy)-6-{[3-(trifluoromethyl)phenyl]amino}-1,3,5-triazin-2-yl]benzamide

LCMS: M+1=485.5; 1H NMR, DMSO-d6 δ, ppm: 3.00 s (6H); 5.15 m (2H); 7.43 d (1H); 7.54-7.68 m (3H); 7.75 d (1H); 8.09 s (1H); 8.14-8.47 m (2H); 10.35 s (1H).

### 30. 4-[4-(5-methyl-1,3,4-oxadiazol-2-yl)phenyl]-6-(2,2,2-trifluoroethoxy)-N-[3-(trifluoromethyl)phenyl]-1,3,5-triazin-2-amine

LCMS: M+1=497.1; 1H NMR, DMSO-d6 δ, ppm: 2.60 s (3H); 5.15 m (2H); 7.44 d (1H); 7.62 t (1H); 7.98 d (1H); 8.16 d (2H); 8.28 s (1H); 8.56 d (2H); 10.50 s (1H).

### 31. 2-fluoro-N,N-dimethyl-5-[4-(2,2,2-trifluoroethoxy)-6-{[3-(trifluoromethyl)phenyl]amino}-1,3,5-triazin-2-yl]benzamide

LCMS: M+1=504.3; 1H NMR, DMSO-d6 δ, ppm: 2.90 s (6H); 5.15 m (2H); 7.38-7.50 m (2H); 7.61 t (1H); 7.94 d (1H); 8.27 s (1H); 8.37 d (1H); 8.47 d (1H); 10.40 s (1H).

### 32. 2-fluoro-N,N-dimethyl-4-[4-(2,2,2-trifluoroethoxy)-6-{[3-(trifluoromethyl)phenyl]amino}-1,3,5-triazin-2-yl]benzamide

LCMS: M+1=504.1; 1H NMR, DMSO-d6 δ, ppm: 5.15 m (2H); 7.45 d (1H); 7.52-7.66 m (2H); 7.96 d (1H); 8.12-8.30 m (3H); 10.45 s (1H).

### 33. N-{3-[4-(2,2,2-trifluoroethoxy)-6-{[3-(trifluoromethyl)phenyl]amino}-1,3,5-triazin-2-yl]phenyl}methanesulfonamide

LCMS: M+1=508.3; 1H NMR, DMSO-d6 δ, ppm: 3.05 m (3H); 5.15 m (2H); 7.42-7.68 m (4H); 8.04-8.22 m (3H); 8.32 s (1H); 9.60 s (1H); 10.45 s (1H).

### 34. N-methyl-3-[4-(2,2,2-trifluoroethoxy)-6-{[3-(trifluoromethyl)phenyl]amino}-1,3,5-triazin-2-yl]benzenesulfonamide

LCMS: M+1=508.3; 1H NMR, DMSO-d6 δ, ppm: 5.15 m (2H); 7.28 s (1H); 7.45 d (1H); 7.62 t (1H); 7.80 t (1H); 8.02 t (2H); 8.23 s (1H); 8.60 d (1H); 8.80 s (1H); 10.50 s 1H).

### 35. N-methyl-4-[4-(2,2,2-trifluoroethoxy)-6-{[3-(trifluoromethyl)phenyl]amino}-1,3,5-triazin-2-yl]benzenesulfonamide

LCMS: M+1=508.3; 1H NMR, DMSO-d6 δ, ppm: 2.95 s (3H); 5.15 m (2H); 7.32 d (1H); 7.45 d (1H); 7.63 t (1H); 7.9-8.04 m (3H); 8.27 s (1H); 8.56 d (2H); 10.50 s (1H).

### 36. 4-[4-(pyrrolidin-1-ylsulfonyl)phenyl]-6-(2,2,2-trifluoroethoxy)-N-[3-(trifluoromethyl)phenyl]-1,3,5-triazin-2-amine

LCMS: M+1=548.6; 1H NMR, DMSO-d6 δ, ppm: 1.75 m (4H); 3.30 m (4H); 5.15 m (2H); 7.44 d (1H); 7.62 t (1H); 7.94-8.04 m (3H); 8.23 s (1H); 8.56 d (2H); 10.50 s (1H).

### 37. 4-[3-(pyrrolidin-1-ylsulfonyl)phenyl]-6-(2,2,2-trifluoroethoxy)-N-[3-(trifluoromethyl)phenyl]-1,3,5-triazin-2-amine

LCMS: M+1=548.6; 1H NMR, DMSO-d6 δ, ppm: 1.70 m (4H); 3.20 m (4H); 5.15 m (2H); 7.46 d (1H); 7.62 t (1H); 7.82 t (1H); 7.96-8.10 m (2H); 8.22 s (1H); 8.66 d (1H); 8.78 s (1H); 10.50 s (1H).

### 38. N-[2-(piperidin-1-yl)ethyl]-4-[4-(2,2,2-trifluoroethoxy)-6-{[3-(trifluoromethyl)phenyl]amino}-1,3,5-triazin-2-yl]benzamide

LCMS: M+1=568.5; 1H NMR (DMSO-d6, 90° C., ppm): =1.59 m (2H), 1.81 m (4H), 3.34 m (6H), 3.71q (2H), 4.98 m (2H), 7.62 t (1H), 7.78 dd (2H), 8.34 dd (4H), 8.38 s (1H), 8.62 s (1H), 10.52 s (1H).

### 39. N-(4-fluorophenyl)-3-(4-(2,2,2-trifluoroethoxy)-6-{[3-(trifluoromethyl)phenyl]amino}-1,3,5-triazin-2-yl)benzamide

1H NMR (DMSO-d6, 90° C., ppm): d=3.58 s (1H, NH), 5.16 q (2H, CH2), 7.18 t (2H, Ar), 7.44 d (1H, Ar), 7.62 t (1H, Ar), 7.72 t (1H, Ar), 7.80 t (2H, Ar), 8.02 d (1H, Ar), 8.18 d (1H, Ar), 8.28 s (1H, Ar), 8.56 d (1H, Ar), 8.94 s (1H, Ar), 10.22 s (1H, Ar), 10.46 s (1H, Ar); LC-MS [M+1]: calc'd: 551.4; obs'd: 552.6.

### 40. N-phenyl-3-(4-(2,2,2-trifluoroethoxy)-6-{[3-(trifluoromethyl)phenyl]amino}-1,3,5-triazin-2-yl)benzamide

1H NMR (DMSO-d6, 90° C., ppm): d=3.58 s (1H, NH), 5.16 q (2H, CH2), 7.12 t (1H, Ar), 7.38 m (3H, Ar), 7.70 m (4H, Ar), 8.00 t (1H, Ar), 8.18 t (1H, Ar), 8.28 d (1H, Ar), 8.56 d (1H, Ar), 8.94 s (1H, Ar), 10.16 s (1H, Ar), 10.48 s (1H, Ar). LC-MS [M+1]: calc'd: 533.4; obs'd: 534.2.

### 41. N-(4-fluorophenyl)-4-[4-(2,2,2-trifluoroethoxy)-6-{[3-(trifluoromethyl)phenyl]amino}-1,3,5-triazin-2-yl]benzamide

1H NMR (DMSO-d6, 90° C., ppm): d=3.58 s (1H, NH), 5.16 q (2H, CH2), 7.18 t (2H, Ar), 7.44 d (1H, Ar), 7.62 t (1H, Ar), 7.78 t (2H, Ar), 7.98 d (1H, Ar), 8.12 d (2H, Ar), 8.32 s (1H, Ar), 8.50 d (2H, Ar), 10.18 s (1H, Ar), 10.46 s (1H, Ar). LC-MS [M+1]: calc'd: 551.4; obs'd: 552.4.

### 42. N-phenyl-4-[4-(2,2,2-trifluoroethoxy)-6-{[3-(trifluoromethyl)phenyl]amino}-1,3,5-triazin-2-yl]benzamide

1H NMR (DMSO-d6, 90° C., ppm): d=4.00 s (1H, NH), 5.16 q (2H, CH2), 7.10 t (3H, Ar), 7.34 m (3H, Ar), 7.46 d (1H, Ar), 7.64 t (1H, Ar), 7.80 d (2H, Ar), 7.98 d (1H, Ar), 8.14 d (2H, Ar), 8.32 s (1H, Ar), 8.52 d (2H, Ar), 10.16 s (1H, Ar), 10.48 s (1H, Ar). LC-MS [M+1]: calc'd: 533.4; obs'd: 534.4.

### 43. 4-{3-[(4-propylpiperazin-1-yl)carbonyl]phenyl}-6-(2,2,2-trifluoroethoxy)-N-[3-(trifluoromethyl)phenyl]-1,3,5-triazin-2-amine

1H NMR (DMSO-d6, 90° C., ppm): d=0.88 t (3H, CH3), 1.46 m (2H, CH2), 2.32 t (2H, CH2), 2.42 t (4H, 2CH2), 3.52 t (4H, 2CH2), 5.14 q (2H, CH2), 7.44 d (1H, Ar), 7.62 t (3H, Ar), 7.96 d (1H, Ar), 8.30 s (1H, Ar), 8.38 s (1H, Ar), 8.46 t (1H, NH), 10.42 s (1H, 1NH). LC-MS [M+1]: calc'd: 568.5; obs'd: 569.6.

### 44. ethyl 4-[3-(4-(2,2,2-trifluoroethoxy)-6-{[3-(trifluoromethyl)phenyl]amino}-1,3,5-triazin-2-yl)benzoyl]piperazine-1-carboxylate

1H NMR (DMSO-d6, 90° C., ppm): d=1.22 t (3H, CH3), 3.50 m (8H, 4CH2), 4.10 q (2H, CH2), 5.16 q (2H, CH2), 7.44 d (1H, Ar), 7.62 t (3H, Ar), 7.96 d (1H, Ar), 8.28 s (1H, Ar), 8.38 s (1H, NH), 8.46 t (1H, CH), 10.44 s (1H, NH). LC-MS [M+1]: calc'd: 598.5; obs'd: 599.5.

### 45. 3-{4-[3-(4-(2,2,2-trifluoroethoxy)-6-{[3-(trifluoromethyl)phenyl]amino}-1,3,5-triazin-2-yl)benzoyl]piperazin-1-yl}propanenitrile

1H NMR (DMSO-d6, 90° C., ppm): d=2.54 t (4H, 2CH2), 2.65 (4H, 2CH2), 3.52 t (4H, 2CH2), 5.16 q (2H, CH2), 7.44 d (1H, Ar), 7.64 m (3H, Ar), 7.98 d (1H, Ar), 8.30 s (1H, Ar), 8.38 s (1H, NH), 8.46 t (1H, Ar), 10.42 s (1H, NH). LC-MS [M+1]: calc'd: 579.5; obs'd: 580.5.

### 46. [4-(propan-2-yl)piperazin-1-yl]{3-[4-(2,2,2-trifluoroethoxy)-6-{[3-(trifluoromethyl)phenyl]amino}-1,3,5-triazin-2-yl]phenyl}methanone

1H NMR (DMSO-d6, 90° C., ppm): d=1.04 d (6H, 2CH3), 2.58 t (4H, 2CH2), 2.80 m (1H, CH), 3.56 t (4H, 2CH2), 5.16 q (2H, CH2), 7.44 d (2H, Ar), 7.64 t (3H, Ar), 7.96 d (1H, Ar), 8.30 s (1H, Ar), 8.38 s (1H, Ar), 8.46 t (1H, NH), 10.44 s (1H, 1NH). LC-MS [M+1]: calc'd: 568.5; obs'd: 569.3.

### 47. [4-(2-methylpropyl)piperazin-1-yl]{3-[4-(2,2,2-trifluoroethoxy)-6-{[3-(trifluoromethyl)phenyl]amino}-1,3,5-triazin-2-yl]phenyl}methanone

1H NMR (DMSO-d6, 90° C., ppm): d=0.88 d (6H, 2CH3), 1.78 m (1H, CH), 2.12 d (2H, CH2), 2.40 q (4H, 4CH), 2.96 t (4H, 2 CH2), 3.54 t (4H, 2 CH2), 5.16 q (2H, CH2), 7.44 d (1H, Ar), 7.62 t (3H, Ar), 7.96 d (1H, Ar), 8.28 s (1H, Ar), 8.38 s (1H, NH), 8.46 t (1H, CH), 10.44 s (1H, NH). LC-MS [M+1]: calc'd: 582.5; obs'd:

### 48. (4-propyl piperazin-1-yl){4-[4-(2,2,2-trifluoroethoxy)-6-{[3-(trifluoromethyl)phenyl]amino}-1,3,5-triazin-2-yl]phenyl}methanone

1H NMR (DMSO-d6, 90° C., ppm): d=0.94 t (3H, CH3), 1.24 t (2H, CH2), 1.70 q (2H, CH2), 1.80 t (4H, 2CH2), 3.02 t (2H, CH2), 3.12 q (2H, CH2), 3.24 m (4H, 2CH2), 5.16 q (2H, CH2), 7.46 d (1H, Ar), 7.62 t (3H, Ar), 7.98 d (1H, Ar), 8.30 s (1H, Ar), 8.46 s (2H, Ar), 10.46 s (1H, NH). LC-MS [M+1]: calc'd: 568.5; obs'd: 569.5.

### 49. ethyl 4-({4-[4-(2,2,2-trifluoroethoxy)-6-{[3-(trifluoromethyl)phenyl]amino}-1,3,5-triazin-2-yl]phenyl}carbonyl)piperazine-1-carboxylate

1H NMR (DMSO-d6, 90° C., ppm): d=1.22 t (3H, CH3), 3.50 m (8H, 4CH2), 4.10 q (2H, CH2), 5.16 q (2H, CH2), 7.46 d (1H, Ar), 7.62 t (3H, Ar), 7.98 d (1H, Ar), 8.28 s (1H, Ar), 8.46 d (2H, Ar). LC-MS [M+1]: calc'd: 598.5; obs'd: 599.3.

### 50. [4-(2-methylpropyl)piperazin-1-yl]{4-[4-(2,2,2-trifluoroethoxy)-6-{[3-(trifluoromethyl)phenyl]amino}-1,3,5-triazin-2-yl]phenyl}methanone

1H NMR (DMSO-d6, 90° C., ppm): d=0.98 d (6H, 2CH3), 2.06 m (1H, CH), 2.82 d (2H, CH2), 3.14 t (4H, 2CH2), 3.76 t (4H, 2CH2), 5.16 q (2H, CH2), 7.46 d (1H, Ar), 7.62 t (3H, Ar), 7.98 d (1H, Ar), 8.28 s (1H, Ar), 8.48 d (2H, Ar), 10.46 s (1H, NH). LC-MS [M+1]: calc'd: 582.5; obs'd: 583.5.

### 51. 4-(naphthalen-2-yl)-6-(2,2,2-trifluoroethoxy)-N-[3-(trifluoromethyl)phenyl]-1,3,5-triazin-2-amine

LCMS: M+1=465.6 NMR 1H, DMSO-d6 δ, ppm: 5.17 m (2H); 7.44 d (1H); 7.62 m (3H); 8.02 m (4H); 8.42 m (2H); 9.00 s (1H); 10.40 bs (1H).

### 52. 4-(4-phenoxyphenyl)-6-(2,2,2-trifluoroethoxy)-N-(3-(trifluoromethyl)phenyl)-1,3,5-triazin-2-amine

1H NMR (DMSO, ppm) 5.02 m (2H), 7.18 d (4H), 7.22 m (1H), 7.42 m (3H), 7.6 m (1H), 8.0 d (1H), 8.24 s (1H), 8.42 d (2H), 10.42 s (1H); LCMS: M+1=507

### 53. N-{3-[4-(2,2,2-trifluoroethoxy)-6-{[3-(trifluoromethyl)phenyl]amino}-1,3,5-triazin-2-yl]phenyl}acetamide

LCMS: M+1=472.6 NMR 1H, DMSO-d6 δ, ppm: 2.10 s (3H); 5.14 m (2H); 7.45 m (2H); 7.63 t (1H); 7.83 d (1H); 8.06 d (2H); 8.23 s (1H); 8.63 s (1H); 9.80 bs (1H).

### 54. 2,4-difluoro-N-{3-[4-(2,2,2-trifluoroethoxy)-6-{[3-(trifluoromethyl)phenyl]amino}-1,3,5-triazin-2-yl]phenyl}benzamide

LCMS: M+1=570.6 NMR 1H, DMSO-d6 δ, ppm: 5.14 m (2H); 7.25 m (2H); 7.42 d (1H); 7.60 m (2H); 7.80 m (1H); 7.94 d (1H); 8.06 d (1H); 8.16 d (1H); 8.27 s (1H); 8.83 s (1H); 10.10 bs (1H); 10.40 bs (1H).

### 55. 3-chloro-4-fluoro-N-{3-[4-(2,2,2-trifluoroethoxy)-6-{[3-(trifluoromethyl)phenyl]amino}-1,3,5-triazin-2-yl]phenyl}benzamide

LCMS: M+1=585.9 NMR 1H, DMSO-d6 δ, ppm: 5.14 m (2H); 7.40-7.65 m (4H); 8.08 m (3H); 8.15-8.30 m (3H); 8.83 s (1H); 10.10 bs (1H); 10.40 bs (1H).

### 56. 4-(propan-2-yl)-N-{3-[4-(2,2,2-trifluoroethoxy)-6-{[3-(trifluoromethyl)phenyl]amino}-1,3,5-triazin-2-yl]phenyl}benzamide

LCMS: M+1=576.6 NMR 1H, DMSO-d6 δ, ppm: 1.29 d (6H); 5.14 m (2H); 7.40-7.65 m (5H); 7.92-8.15 m (5H); 8.27 bs (1H); 8.83 bt (1H); 10.10 bs (1H); 10.40 bs (1H).

### 57. 3-cyano-N-{3-[4-(2,2,2-trifluoroethoxy)-6-{[3-(trifluoromethyl)phenyl]amino}-1,3,5-triazin-2-yl]phenyl}benzamide

LCMS: M+1=559.6 NMR 1H, DMSO-d6 δ, ppm: 5.14 m (2H); 7.40 d (1H); 7.60 m (2H); 7.78 t (1H); 8.02 m (3H); 8.16 d (1H); 8.32 m (2H); 8.42 s (1H); 8.83 s (1H); 10.40 d (2H).

### 58. 4-(3″-methoxybiphenyl-3-yl)-6-(2,2,2-trifluoroethoxy)-N-[3-(trifluoromethyl)phenyl]-1,3,5-triazin-2-amine

LCMS: M+1=521.6; 1H NMR, DMSO-d6 δ, ppm: 3.85 s (3H); 5.15 m (2H); 7.01 d (1H); 7.20-7.32 m (2H); 7.35-7.48 m (2H); 7.55-7.70 m (2H); 7.89-8.00 m (2H); 8.30 s (1H); 8.35 d (1H); 8.62 s (1H); 10.41 s (1H).

### 59. methyl 3′-(4-(2,2,2-trifluoroethoxy)-6-(3-(trifluoromethyl)phenylamino)-1,3,5-triazin-2-yl)biphenyl-4-carboxylate

1H NMR (DMSO, ppm) 3.96 s (3H), 5.02 m (2H), 7.42 d (1H), 7.62 m (2H), 7.82 d (2H), 7.88 d (2H), 8.16 d (2H), 8.32 s (1H), 8.44 d (1H), 8.68 d (1H), 10.32 s (1H); LCMS: M+1=549

### 60. 4-(3′-fluorobiphenyl-3-yl)-6-(2,2,2-trifluoroethoxy)-N-(3-(trifluoromethyl)phenyl)-1,3,5-triazin-2-amine

1H NMR (DMSO, ppm) 5.02 m (2H), 7.2 m (1H), 7.60 m (6H), 7.98 m (2H), 8.38 s (1H), 8.42 d (1H), 8.62 s (1H), 10.42 s (1H); LCMS: M+1=509

### 61. 4-(3′-fluorobiphenyl-3-yl)-6-(2,2,2-trifluoroethoxy)-N-(3-(trifluoromethyl)phenyl)-1,3,5-triazin-2-amine

1H NMR (DMSO, ppm) 3.98 s (3H), 5.02 m (2H), 7.4 d (1H), 7.6 m (3H), 7.98 m (4H), 8.22 d (2H), 8.42 d (1H), 8.64 s (1H), 10.20 bs (1H); LCMS: M+1=549

### Generic Synthesis of Intermediates and Final Compounds for Table 40

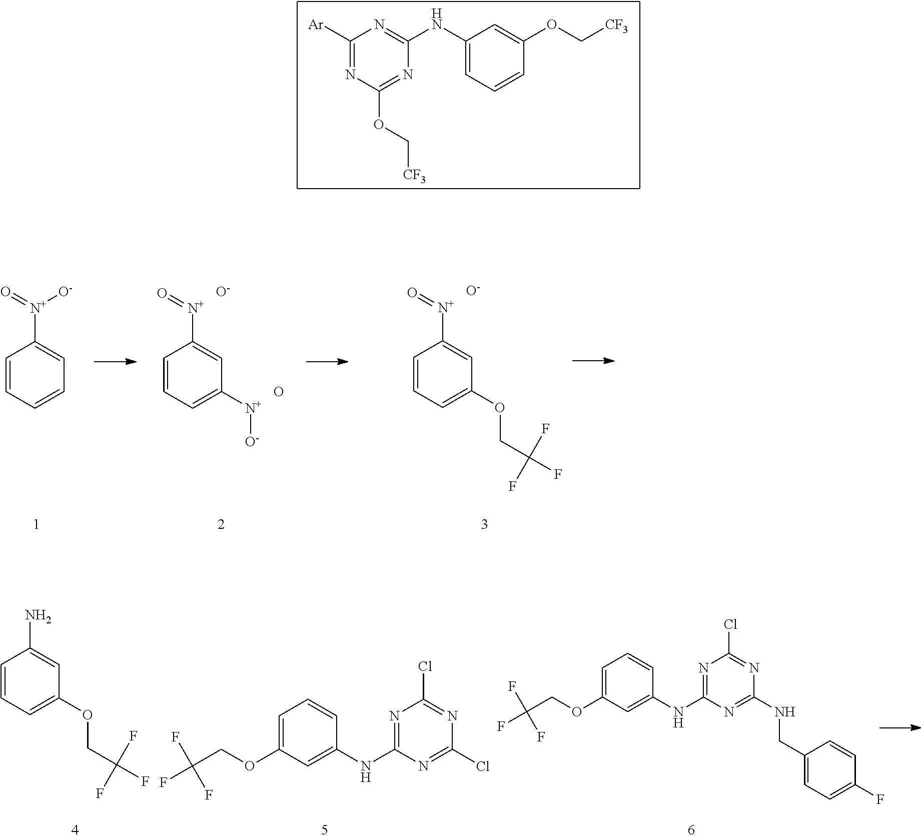

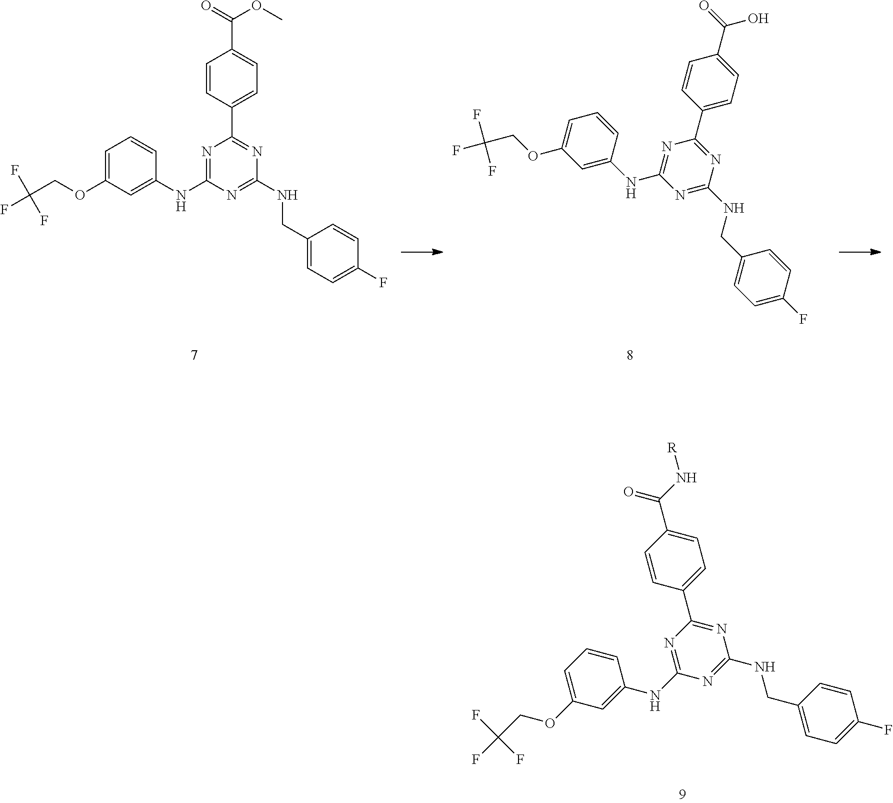

95 g of intermediate 2 was obtained according to the following procedure: The mixture of 112 ml of concentrated sulfuric acid and 80 ml of fuming nitric acid was added dropwise to 100 g of intermediate 1 at 60° C., then stirred 1 hour at 80° C. and diluted onto crushed ice. The precipitate obtained was filtered off and washed with water, ethanol and ether afforded 2 as yellow solid. Yield 95 g. NMR ¹H, DMSO-d₆ δ, ppm: 7.98 t (1H); 8.65 dd (2H); 8.87 s (1H).

100 g of intermediate 3 was obtained according to the following procedure: To 400 ml of DMF at r.t. 24.2 g of NaH (60% suspension in mineral oil) was added. After 30 min of stirring, 110.1 g of trifluoroethanol was added dropwise to the mixture. After 1 hour of stirring, 95 g of intermediate 2 was added and the mixture was heated for 7 hours, diluted with 1% aqueous HCl, and the precipitate obtained was filtered and washed with water to afford 3 as brown solid. Yield 100 g. NMR ¹H, DMSO-d₆ δ, ppm: 4.87 m (2H); 7.51 m (1H); 7.62 t (1H); 7.89 m (2H).

6 g of intermediate 4 was obtained according to the following procedure: 10 g of intermediate 3 was hydrogenated over Pd/C in ethanol at 2 atm. and r.t. for 48 hours, filtered, the solvent was removed to afford 4 as brown oil. Yield 6.1 g. NMR ¹H, DMSO-d₆ δ, ppm: 4.49 m (2H); 5.00 bs (2H); 6.21 d (1H); 6.27 s (1H); 6.35 d (1H); 6.97 t (1H).

Intermediate 5 was obtained according to the following procedure: To a stirred solution of 2,4,6-trichlorotriazine (5.8 g.) in 200 ml of dry dioxane at 0° C., a solution of 4 (6 g) in 50 ml of dioxane was added dropwise, maintaining the temperature under 15° C. After 4 hours of stirring at r.t. the reaction mixture was concentrated. The residue was washed with 200 ml of CH₂Cl₂ and filtered. The solvent was removed and the residue was washed with hexane to afford 5 as white solid Yield 9.2 g.

Intermediate 6 was obtained according to the following procedure: To a stirred solution of 5 (9.2 g.) in 250 ml of dry dioxane at 0° C., a solution of p-fluorobenzylamine (3.40 g) in 50 ml of dioxane was added dropwise, maintaining the temperature under 15° C. After 4 hours of stirring at r.t. the reaction mixture was concentrated. The residue was washed with 200 ml of CH₂Cl₂ and filtered. The solvent was removed and residue was washed with hexane afforded 5 as white solid Yield 3.2 g. LCMS [M+1]=428.1

Intermediate 7 was obtained according to the standard procedure: compound 6 (0.125 mmol) was suspended in dioxane (5 ml) in the presence of K₂CO₃ (0.035 g, 0.251 mmol). This mixture was treated with appropriate amines (0.125 mmol) at rt. The reaction mixture was then stirred for 30-40 min at 70-80° C. The reaction mixture was cooled, poured into water and extracted with CH₂Cl₂ or CHCl₃. The organic extract was separated, dried over MgSO₄, filtered and concentrated. Noncrystalline products were purified by silica gel chromatography with appropriate eluents. Yield after column chromatography (dichloromethane) 1 g.

Intermediate 8 was obtained according to the following procedure: Intermediate 7 (1 g) and KOH (0.15 g) were heated at 100° C. for 10 hours in 70 ml of water, acidified with HCl, and the precipitate obtained was filtered off and washed with water to afford 8 as white solid. LCMS: H+1=514.5

Compound 9 was synthesized according to the following procedure: Acid 8 (1.3 mmol) was dissolved in 2 ml of dioxane and treated with CDI (1.3 mmol). The reaction mixture was stirred for 1 hour at an ambient temperature, at which time it was treated with a solution of amine (1.4 mmol). This mixture was stirred at 70° C. for 8 hours, then cooled and concentrated under reduced pressure. The residue was washed with 10% aqueous NaHCO₃, then with water, dried, and purified by using column chromatography with CH₂Cl₂ as eluent.

TABLE 40

| En | Structure | IUPAC Name | MW | Formula |
|---|---|---|---|---|
| 1 | 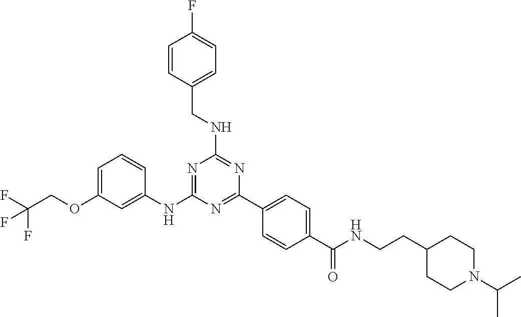 | 4-(4-[(4-fluorobenzyl)amino]-6- {[3-(2,2,2- trifluoroethoxy)phenyl]amino}- 1,3,5-triazin-2-yl)-N-{2-[1- (propan-2-yl)piperidin-4- yl]ethyl}benzamide | 665.7 | C35H39F4N7O2 |
| 2 | 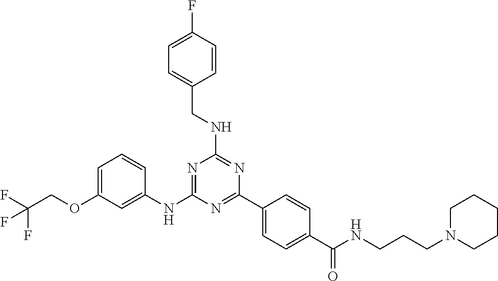 | 4-(4-[(4-fluorobenzyl)amino]-6- {[3-(2,2,2- trifluoroethoxy)phenyl]amino}- 1,3,5-triazin-2-yl)-N-[3- (piperidin-1-yl)propyl]benzamide | 637.7 | C33H35F4N7O2 |
| 3 | 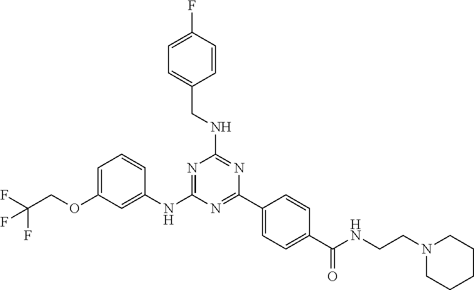 | 4-(4-[(4-fluorobenzyl)amino]-6- {[3-(2,2,2- trifluoroethoxy)phenyl]amino}- 1,3,5-triazin-2-yl)-N-[2- (piperidin-1-yl)ethyl]benzamide | 623.6 | C32H33F4N7O2 |

### Procedures and Analytical Data for Table 40.

The compounds were prepared by the above procedures.

### 1. 4-(4-[(4-fluorobenzyl)amino]-6-{[3-(2,2,2-trifluoroethoxy)phenyl]amino}-1,3,5-triazin-2-yl)-N-{2-[1-(propan-2-yl)piperidin-4-yl]ethyl}benzamide

LCMS: M+1=666.7; NMR 1H, DMSO-d6 δ, ppm: 1.01 d (6H); 1.30 m (3H); 1.54 m (2H); 1.74 d (2H); 2.20 t (2H); 2.80 m (3H); 3.35 m (2H); 4.65 m (4H); 6.73 dd (1H); 7.10 t (2H); 7.25 t (1H); 7.45 m (3H); 7.65 t (1H); 7.92 m (3H); 8.16 t (1H); 8.36 d (2H); 9.30 s (1H).

### 2. 4-(4-[(4-fluorobenzyl)amino]-6-{[3-(2,2,2-trifluoroethoxy)phenyl]amino}-1,3,5-triazin-2-yl)-N-[3-(piperidin-1-yl)propyl]benzamide

LCMS: M+1=638.7; NMR ¹H, DMSO-d₆ δ, ppm: 1.44 m (2H); 1.60 m (4H); 1.81 m (2H); 2.52 m (6H); 3.35 m (2H); 4.65 m (4H); 6.76 dd (1H); 7.10 t (2H); 7.25 t (1H); 7.45 m (3H); 7.65 t (1H); 7.95 m (3H); 8.30 m (3H); 9.30 s (1H).

### 3. 4-(4-[(4-fluorobenzyl)amino]-6-{[3-(2,2,2-trifluoroethoxy)phenyl]amino}-1,3,5-triazin-2-yl)-N-[2-(piperidin-1-yl)ethyl]benzamide

LCMS: M+1=624.7; NMR ¹H, DMSO-d₆ δ, ppm: 1.41 m (2H); 1.55 m (4H); 2.52 m (6H); 3.35 m (2H); 4.65 m (4H); 6.76 dd (1H); 7.10 t (2H); 7.25 t (1H); 7.45 m (3H); 7.65 t (1H); 7.90 m (3H); 8.10 t (1H); 8.38 d (2H); 9.30 s (1H).

### Generic Synthesis of Intermediates and Final Compounds for Table 41

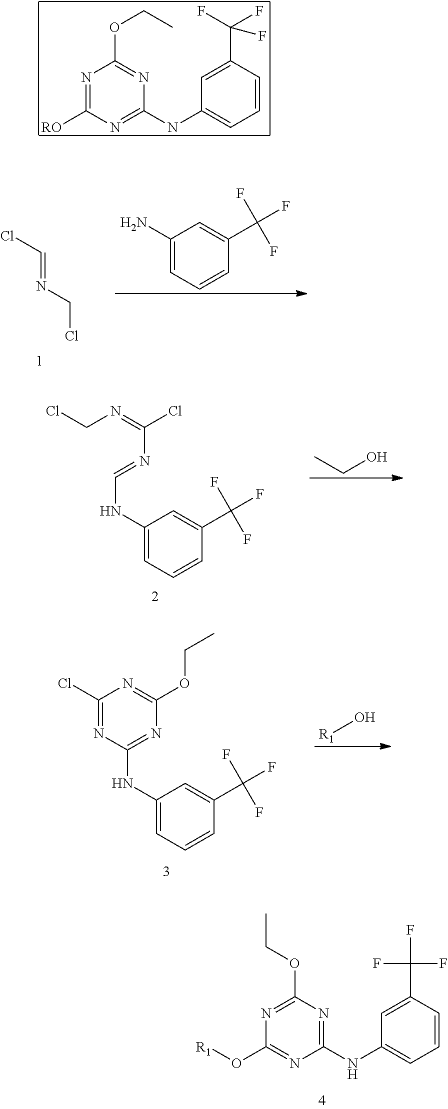

Preparation of 2,4-dichloro-6-(N-3-trifluoromethylanilino)-1,3,5-triazine 2. To a stirred solution of 2,4,6-trichlorotriazine 1 (5 g.) and anhydrous sodium acetate (2.46 g) in dry dioxane (200 ml) was added a solution of aniline (4.39 g) in 50 ml of dioxane dropwise while the reaction temperature was maintained under 15° C. After 1 hour of stirring at r.t., the solvent was removed under reduced pressure and the solid residue was treated successively with saturated aqueous K₂CO₃ solution and then with benzene (2*20 ml). The white solid obtained was dissolved in 100 ml of chloroform; insoluble material was filtered off. Filtrate was dried over sodium sulfate and concentrated under reduced pressure. 2,4-Dichloro-6-(N-3-trifluoro-methylanilino)-1,3,5-triazine 2 was obtained as a white solid in 30% yield (2.56 g).

Preparation of 4-chloro-6-ethoxy-N-[3-(trifluoromethyl)phenyl]-1,3,5-triazine-2-amine 3. Sodium (0.075 g, 3.24 mmol) was carefully dissolved in dry ethanol (20 ml) at a r.t. 2,4-Dichloro-6-(N-3-trifluoromethyl-anilino)-1,3,5-triazine 2 (1 g, 3.24 mmol) was dissolved separately in dry ethanol (30 ml), cooled to −30° C. and treated with the sodium ethoxide solution. The reaction mixture was stirred for 3 hours at r.t. LCMS analysis of the reaction mixture demonstrated no starting 2,4-dichloro-6-(N-3-trifluoromethyl-anilino)-1,3,5-triazine. The main product of the reaction was 2-ethoxy-4-chloro-6-(N-3-trifluoromethyl-anilino)-1,3,5-triazine (M+1=319.4)—90%. Minor components were 2,4-diethoxy-6-(N-3-trifluoromethylanilino)-1,3,5-triazine and 2,4-dioxy-6-(N-3-trifluoromethyl-anilino)-1,3,5-triazine (M+1=329.0 and M+1=273.0, correspondingly)—10% total. The reaction mixture was concentrated and the crude product 3 was recrystallized from hexane. Yield was 60% (0.62 g).

Preparation of 4. Alcohol (0.37 mmol) was dissolved in 1,4-dioxane (5 ml) and treated with potassium tert-butoxide (0.05 g, 0.45 mmol). The mixture was stirred for 2 h at 60° C. and then cooled to rt. 4-chloro-6-ethoxy-N-[3-(trifluoromethyl)phenyl]-1,3,5-triazine-2-amine 3 (0.12 g, 0.37 mmol) was added to this solution and the reaction mixture was heated to 60° C. for 1 h. The reaction mixture was cooled, diluted with water (100 ml) and extracted with ethyl acetate. The organic extract was separated, dried over MgSO₄ and concentrated. The crude product was purified by silica gel chromatography with ethyl acetate/hexanes (1:40) as eluent.

TABLE 41

| En | Structure | IUPAC Name | MW | Formula |
|---|---|---|---|---|
| 1 | 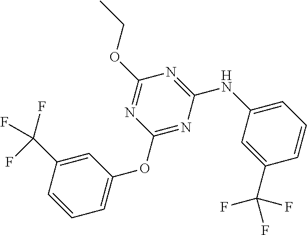 | 4-ethoxy-6-[3- (trifluoromethyl)phenoxy]-N-[3- (trifluoromethyl)phenyl]-1,3,5- triazin-2-amine | 444.3 | C₁₉H₁₄F₆N₄O₂ |
| 2 | 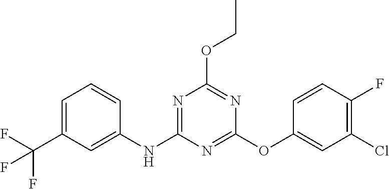 | 4-(3-chloro-4-fluorophenoxy)-6- ethoxy-N-[3- (trifluoromethyl)phenyl]-1,3,5- triazin-2-amine | 428.8 | C₁₈H₁₃ClF₄N₄O₂ |
| 3 | 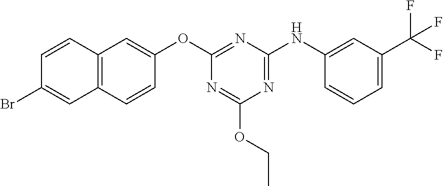 | 4-[(6-bromonaphthalen-2-yl)oxy]- 6-ethoxy-N-[3- (trifluoromethyl)phenyl]-1,3,5- triazin-2-amine | 505.3 | C₂₂H₁₆BrF₃N₄O₂ |
| 4 | 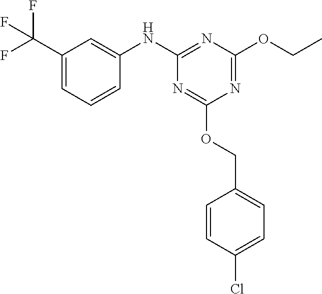 | 4-[(4-chlorobenzyl)oxy]-6-ethoxy- N-[3-(trifluoromethyl)phenyl]- 1,3,5-triazin-2-amine | 424.8 | C₁₉H₁₆ClF₃N₄O₂ |
| 5 | 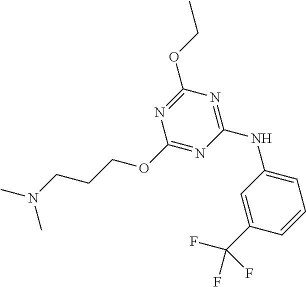 | 4-[3-(dimethylamino)propoxy]-6- ethoxy-N-[3- (trifluoromethyl)phenyl]-1,3,5- triazin-2-amine | 385.4 | C₁₇H₂₂F₃N₅O₂ |
| 6 | 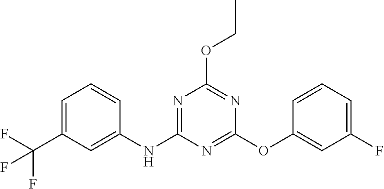 | 4-ethoxy-6-(3-fluorophenoxy)-N- [3-(trifluoromethyl)phenyl]-1,3,5- triazin-2-amine | 394.3 | C₁₈H₁₄F₄N₄O₂ |
| 7 | 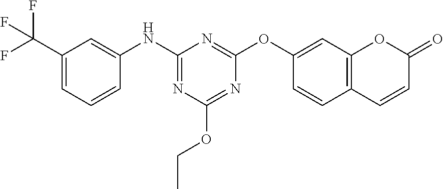 | 7-[(4-ethoxy-6-{[3- (trifluoromethyl)phenyl]amino}- 1,3,5-triazin-2-yl)oxy]-2H- chromen-2-one | 444.4 | C₂₁H₁₅F₃N₄O₄ |
| 8 | 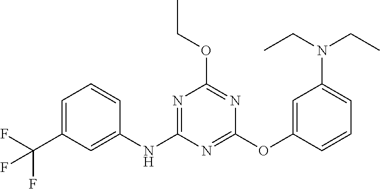 | 4-[3-(diethylamino)phenoxy]-6- ethoxy-N-[3- (trifluoromethyl)phenyl]-1,3,5- triazin-2-amine | 447.5 | C₂₂H₂₄F₃N₅O₂ |
| 9 | 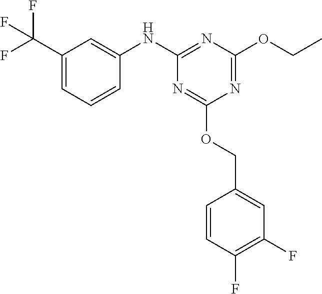 | 4-[(3,4-difluorobenzyl)oxy]-6- ethoxy-N-[3- (trifluoromethyl)phenyl]-1,3,5- triazin-2-amine | 426.3 | C₁₉H₁₅F₅N₄O₂ |
| 10 | 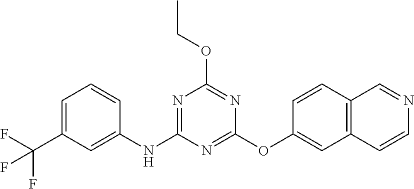 | 4-ethoxy-6-(isoquinolin-6-yloxy)- N-[3-(trifluoromethyl)phenyl]- 1,3,5-triazin-2-amine | 427.4 | C₂₁H₁₆F₃N₅O₂ |

### Procedures and Analytical Data for Table 41.

Entries 1 to 10 were prepared by the methods described above.

### 1. 4-ethoxy-6-[3-(trifluoromethyl)phenoxy]-N-[3-(trifluoromethyl)phenyl]-1,3,5-triazin-2-amine

LCMS: M+1=444.3; 1H NMR (DMSO-d6, 90° C., ppm): d=1.42 t (3H), 4.48 q (2H), 7.80 m (9H).

### 2. 4-(3-chloro-4-fluorophenoxy)-6-ethoxy-N-[3-(trifluoromethyl)phenyl]-1,3,5-triazin-2-amine

LCMS: M+1=428.7; 1H NMR (DMSO-d6, 90° C., ppm): d=1.34 t (3H), 4.42 q (2H), 7.40 m (5H), 7.82 d (1H), 8.04 s (1H), 10.12 s (1H).

### 3. 4-[(6-bromonaphthalen-2-yl)oxy]-6-ethoxy-N-[3-(trifluoromethyl)phenyl]-1,3,5-triazin-2-amine

LCMS: M+1=505.2; 1H NMR (DMSO-d6, 90° C., ppm): d=1.48 t (3H), 4.46 q (2H), 7.32 m (5H), 7.64 m (4H), 7.82 d (1H), 8.08 s (1H).

### 4. 4-[(4-chlorobenzyl)oxy]-6-ethoxy-N-[3-(trifluoromethyl)phenyl]-1,3,5-triazin-2-amine

LCMS: M+1=424.8; 1H NMR (DMSO-d6, 90° C., ppm): d=1.32 t (3H), 4.40 q (2H), 5.40 s (2H), 7.38 d (1H), 7.44 s (4H), 7.48 t (1H), 7.86 d (1H), 8.22 s (1H), 10.42 s (1H).

### 5. 4-[3-(dimethylamino)propoxy]-6-ethoxy-N-[3-(trifluoromethyl)phenyl]-1,3,5-triazin-2-amine

LCMS: M+1=385.3; 1H NMR (DMSO-d6, 90° C., ppm): d=1.48 t (3H), 1.96 m (2H), 2.30 d (5H), 2.48 t (2H), 2.70 s (1H), 4.48 q (4H), 7.36 d (2H), 7.48 t (2H), 7.66 d (1H), 8.12 s (1H).

### 6. 4-ethoxy-6-(3-fluorophenoxy)-N-[3-(trifluoromethyl)phenyl]-1,3,5-triazin-2-amine

LCMS: M+1=394.3; 1H NMR (DMSO-d6, 90° C., ppm): d=1.44 t (3H), 1.62 s (2H), 4.48 q (2H), 7.02 m (3H), 7.48 m (5H).

### 7. 7-[(4-ethoxy-6-{[3-(trifluoromethyl)phenyl]amino}-1,3,5-triazin-2-yl)oxy]-2H-chromen-2-one

LCMS: M+1=444.3; 1H NMR (DMSO-d6, 90° C., ppm): d=0.92 t (1H), 1.28 m (1H), 1.46 t (3H), 4.48 q (2H), 6.48 d (1H), 7.16 d (1H), 7.26 m (6H), 7.46 m (4H), 7.72 d (1H).

### 8. 4-[3-(diethylamino)phenoxy]-6-ethoxy-N-[3-(trifluoromethyl)phenyl]-1,3,5-triazin-2-amine

LCMS: M+1=444.3; 1H NMR (DMSO-d6, 90° C., ppm): d=0.92 t (1H), 1.28 m (1H), 1.46 t (3H), 4.48 q (2H), 6.48 d (1H), 7.16 d (1H), 7.26 m (6H), 7.46 m (4H), 7.72 d (1H).

### 9. 4-[(3,4-difluorobenzyl)oxy]-6-ethoxy-N-[3-(trifluoromethyl)phenyl]-1,3,5-triazin-2-amine

LCMS: M+1=426.3; 1H NMR (DMSO-d6, 90° C., ppm): d=1.36 t (3H), 4.92 q (2H), 5.42 s (2H), 7.42 m (5H), 7.92 d (1H), 8.18 s (1H), 10.02 s (1H).

### 10. 7-[(4-ethoxy-6-{[3-(trifluoromethyl)phenyl]amino}-1,3,5-triazin-2-yl)oxy]-2H-chromen-2-one

LCMS: M+1=427.3; 1H NMR (DMSO-d6, 90° C., ppm): d=1.40 t (3H), 4.48 q (2H), 6.28 s (1H), 6.58 t (1H), 7.08 t (1H), 7.86 m (4H), 8.34 d (2H), 9.16 s (1H), 10.84 s (1H).

### Generic Synthesis of Intermediates and Final Compounds in Table 42 (see Table 31).

TABLE 42

| En | Structure | IUPAC Name | MW | Formula |
|---|---|---|---|---|
| 1 | 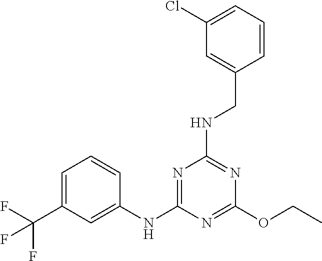 | N-(3-chlorobenzyl)-6-ethoxy-N′-[3- (trifluoromethyl)phenyl]-1,3,5-triazine- 2,4-diamine | 423.8 | C₁₉H₁₇ClF₃N₅O |
| 2 | 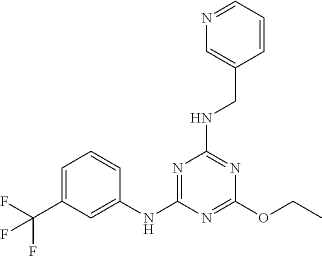 | 6-ethoxy-N-(pyridin-3-ylmethyl)-N′-[3- (trifluoromethyl)phenyl]-1,3,5-triazine- 2,4-diamine | 390.4 | C₁₈H₁₇F₃N₆O |
| 3 | 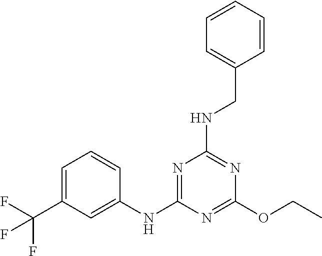 | N-benzyl-6-ethoxy-N′-[3- (trifluoromethyl)phenyl]-1,3,5-triazine- 2,4-diamine | 389.4 | C₁₉H₁₈F₃N₅O |
| 4 | 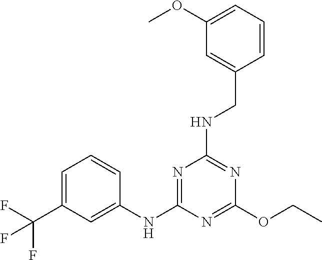 | 6-ethoxy-N-(3-methoxybenzyl)-N′-[3- (trifluoromethyl)phenyl]-1,3,5-triazine- 2,4-diamine | 419.4 | C₂₀H₂₀F₃N₅O₂ |
| 5 | 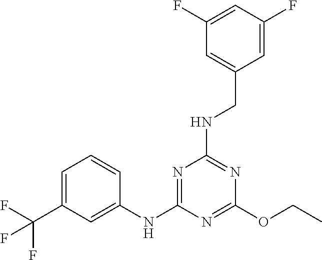 | N-(3,5-difluorobenzyl)-6-ethoxy-N′-[3- (trifluoromethyl)phenyl]-1,3,5-triazine- 2,4-diamine | 425.4 | C₁₉H₁₆F₅N₅O |
| 6 | 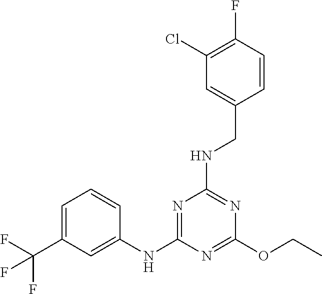 | N-(3-chloro-4-fluorobenzyl)-6-ethoxy- N′-[3-(trifluoromethyl)phenyl]-1,3,5- triazine-2,4-diamine | 441.8 | C₁₉H₁₆ClF₄N₅O |
| 7 | 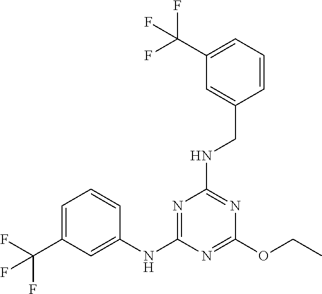 | 6-ethoxy-N-[3- (trifluoromethyl)benzyl]-N′-[3- (trifluoromethyl)phenyl]-1,3,5-triazine- 2,4-diamine | 457.4 | C₂₀H₁₇F₆N₅O |
| 8 | 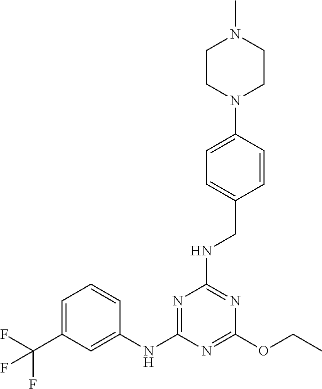 | 6-ethoxy-N-[4-(4-methylpiperazin-1- yl)benzyl]-N′-[3- (trifluoromethyl)phenyl]-1,3,5-triazine- 2,4-diamine | 487.5 | C₂₄H₂₈F₃N₇O |
| 9 | 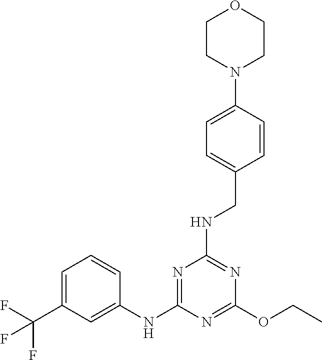 | 6-ethoxy-N-[4-(morpholin-4- yl)benzyl]-N′-[3- (trifluoromethyl)phenyl]-1,3,5-triazine- 2,4-diamine | 474.5 | C₂₃H₂₅F₃N₆O₂ |

### Procedures and Analytical Data for Table 42.

Entries 1 to 9 were prepared by the methods associated with Library 31 (see Table 31).

### 1. N-(3-chlorobenzyl)-6-ethoxy-N′-[3-(trifluoromethyl)phenyl]-1,3,5-triazine-2,4-diamine

LCMS: M+=424.4; 1H NMR CDCl3, ppm: 1.40 s (3H); 4.40 m (2H); 4.60 m (2H); 5.70-6.20 m (1H); 7.7.50 m (8H); 7.55-7.70 m (1H); 7.95-8.15 s (1H)

### 2. 6-ethoxy-N-(pyridin-3-ylmethyl)-N′-[3-(trifluoromethyl)phenyl]-1,3,5-triazine-2,4-diamine

LCMS: M+1=390.3; 1H NMR, DMSO-d6 δ, ppm: 1.3 t (3H); 4.35 m (2H); 4.60 d (2H); 7.1-7.60 m (4H); 7.70 d (1H); 7.85 d (1H); 8.20 s (1H); 8.30 m (2H); 9.30 s (1H).

### 3. N-benzyl-6-ethoxy-N′-[3-(trifluoromethyl)phenyl]-1,3,5-triazine-2,4-diamine

LCMS: M+=390.8; 1H NMR CDCl3, ppm: 1.40 s (3H); 4.40 m (2H); 4.60 m (2H); 5.50-5.80 m (1H); 7.20-7.50 m (10H); 7.55-7.75 m (1H); 7.95-8.25 s (1H)

### 4. 6-ethoxy-N-(3-methoxybenzyl)-N′-[3-(trifluoromethyl)phenyl]-1,3,5-triazine-2,4-diamine

LCMS: M+=420.5 1H NMR, DMSO-d6 δ, ppm: 1.35 m (3H); 3.75 s (3H); 4.35 m (2H); 4.55 m (2H); 6.75 d (1H); 6.90 s (2H); 7.10-7.50 m (4H); 7.95 d (1H); 8.20 s (1H); 9.25 s (1H)

### 5. N-(3,5-difluorobenzyl)-6-ethoxy-N′-[3-(trifluoromethyl)phenyl]-1,3,5-triazine-2,4-diamine

LCMS: M+1=425.3; 1H NMR, DMSO-d6 δ, ppm: 1.35 t (3H); 4.35 m (2H); 4.65 m (2H); 6.82 t (1H); 6.98 d (2H); 7.24 d (1H); 7.42 t (1H); 7.54 t (1H); 7.82 d (1H); 8.18 s (1H); 9.25 s (1H).

### 6. N-(3-chloro-4-fluorobenzyl)-6-ethoxy-N′-[3-(trifluoromethyl)phenyl]-1,3,5-triazine-2,4-diamine

LCMS: M+1=441.8; 1H NMR, DMSO-d6 δ, ppm: 1.35 t (3H); 4.35 m (2H); 4.65 m (2H); 7.22 d (1H); 7.40 t (1H); 7.50-7.72 m (5H); 7.92 d (1H); 8.18 s (1H); 9.25 s (1H).

### 7. 6-ethoxy-N-[3-(trifluoromethyl)benzyl]-N′-[3-(trifluoromethyl)phenyl]-1,3,5-triazine-2,4-diamine

LCMS: M+1=457.3; 1H NMR, DMSO-d6 δ, ppm: 1.35 t (3H); 4.35 m (2H); 4.65 m (2H); 7.22 d (1H); 7.42 t (1H); 7.35-7.70 m (5H); 7.92 d (1H); 8.18 s (1H); 9.25 s (1H).

### 8. 6-ethoxy-N-[4-(4-methylpiperazin-1-yl)benzyl]-N′-[3-(trifluoromethyl)phenyl]-1,3,5-triazine-2,4-diamine

LCMS: M+1=487.5; 1H NMR, DMSO-d6 δ, ppm: 1.35 t (3H); 2.25 s (3H); 2.55 m (4H); 3.15 m (4H); 4.35 m (2H); 4.45 m (2H); 6.88 d (2H); 7.20 d (2H); 7.28 d (1H); 7.48 t (2H); 7.98 d (1H); 8.22 s (1H); 9.35 s (1H).

### 9. 6-ethoxy-N-[4-(morpholin-4-yl)benzyl]-N′-[3-(trifluoromethyl)phenyl]-1,3,5-triazine-2,4-diamine

LCMS: M+1=474.4; 1H NMR, DMSO-d6 δ, ppm: 1.35 t (3H); 3.15 m (4H); 3.75 m (4H); 4.35 m (2H); 4.50 m (2H); 6.75 d (2H); 7.7.30 m (3H); 7.45 t (2H); 7.95 d (1H); 8.20 s (1H); 9.35 s (1H).

### Generic Synthesis of Intermediates and Final Compounds for Table 43

A combination of R2-R4-R6 library was synthesized utilizing combinations of the reactions described above.

TABLE 43

| En | Structure | IUPAC Name | MW | Formula |
|---|---|---|---|---|
| 1 | 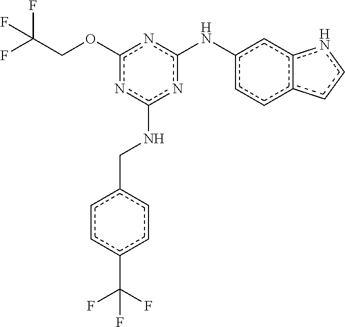 | N-(1H-indol-6-yl)-6-(2,2,2- trifluoro-ethoxy)-N′-[4- (trifluoromethyl)benzyl]- 1,3,5-triazine-2,4-diamine | 482.4 | C21H16F6N6O |
| 2 | 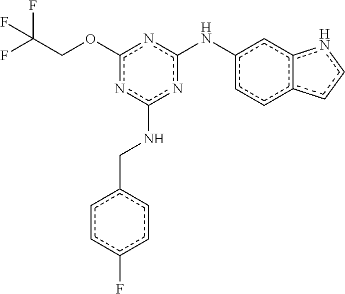 | N-(4-fluorobenzyl)-N′-(1H- indol-6-yl)-6-(2,2,2- trifluoroethoxy)-1,3,5- triazine-2,4-diamine | 432.4 | C20H16F4N6O |
| 3 | 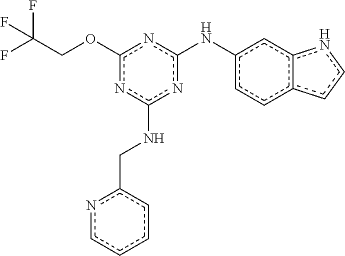 | N-(1H-indol-6-yl)-N′-(pyridin- 2-ylmethyl)-6-(2,2,2- trifluoroethoxy)-1,3,5- triazine-2,4-diamine | 415.4 | Cl9H16F3N7O |
| 4 | 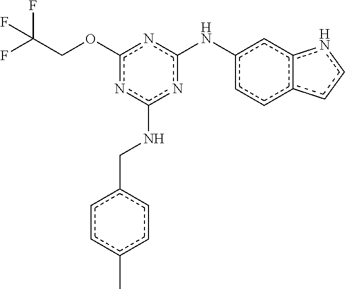 | N-(1H-indol-6-yl)-N′-(4- methylbenzyl)-6-(2,2,2- trifluoroethoxy)-1,3,5- triazine-2,4-diamine | 428.4 | C21H19F3N6O |
| 5 | 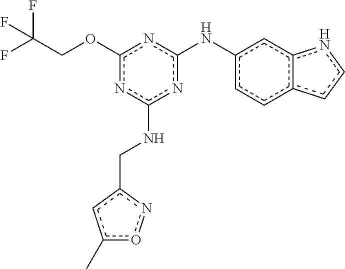 | N-(1H-indol-6-yl)-N′-[(5- methylisoxazol-3-yl)methyl]- 6-(2,2,2-trifluoroethoxy)- 1,3,5-triazine-2,4-diamine | 419.4 | C18H16F3N7O2 |
| 6 | 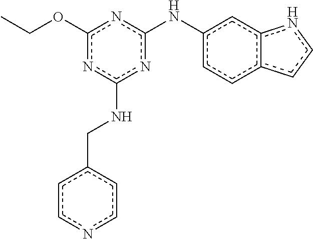 | 6-ethoxy-N-(1H-indol-6-yl)- N′-(pyridin-4-ylmethyl)-1,3,5- triazine-2,4-diamine | 361.4 | C19H19N7O |
| 7 | 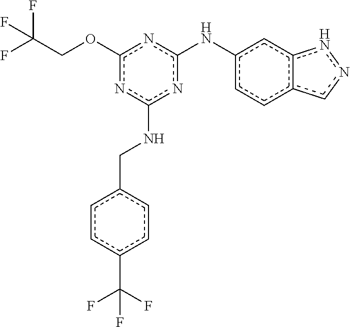 | N-(1H-indazol-6-yl)-6-(2,2,2- trifluoro-ethoxy)-N′-[4- (trifluoromethyl)benzyl]- 1,3,5-triazine-2,4-diamine | 483.4 | C20H15F6N7O |
| 8 | 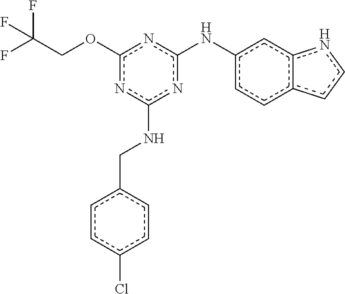 | N-(4-chlorobenzyl)-N′-(1H- indol-6-yl)-6-(2,2,2- trifluoroethoxy)-1,3,5- triazine-2,4-diamine | 448.8 | C20H16ClF3N6O |
| 9 | 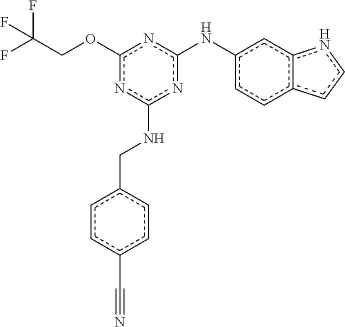 | 4-({[4-(1H-indol-6-ylamino)- 6-(2,2,2-trifluoroethoxy)- 1,3,5-triazin-2- yl]amino}methyl)benzonitrile | 439.4 | C21H16F3N7O |
| 10 | 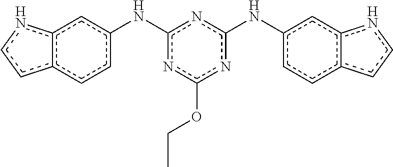 | 6-ethoxy-N,N″-di(1H-indol-6- yl)-1,3,5-triazine-2,4-diamine | 385.4 | C21H19N7O |
| 11 | 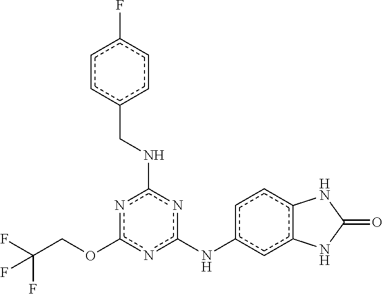 | 5-({4-[(4- fluorobenzyl)amino]-6-(2,2,2- trifluoroethoxy)-1,3,5-triazin- 2-yl}amino)-1,3-dihydro-2H- benzimidazol-2-one | 449.4 | C19H15F4N7O2 |
| 12 | 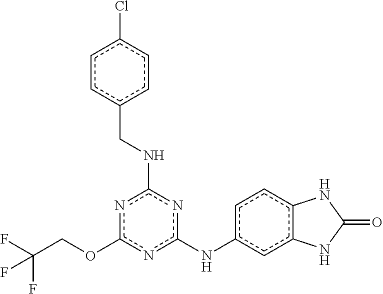 | 5-({4-[(4- chlorobenzyl)amino]-6- (2,2,2-trifluoroethoxy)-1,3,5- triazin-2-yl}amino)-1,3- dihydro-2H-benzimidazol-2- one | 465.8 | C19H15ClF3N7O2 |
| 13 | 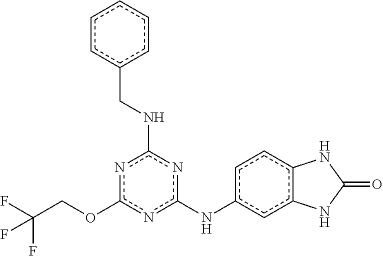 | 5-{[4-(benzylamino)-6-(2,2,2- trifluoroethoxy)-1,3,5-triazin- 2-yl]amino}-1,3-dihydro-2H- benzimidazol-2-one | 431.4 | C19H16F3N7O2 |
| 14 | 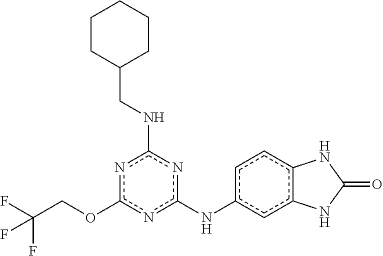 | 5-({4- [(cyclohexylmethyl)amino]-6- (2,2,2-trifluoroethoxy)-1,3,5- thazin-2-yl}amino)-1,3- dihydro-2H-benzimidazol-2-one | 437.4 | C19H22F3N7O2 |
| 15 | 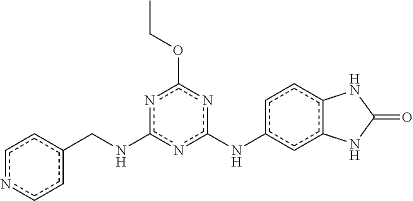 | 5-({4-ethoxy-6-[(pyridin-4- ylmethyl)amino]-1,3,5-triazin- 2-yl}amino)-1,3-dihydro-2H- benzimidazol-2-one | 378.4 | C18H18N8O2 |
| 16 | 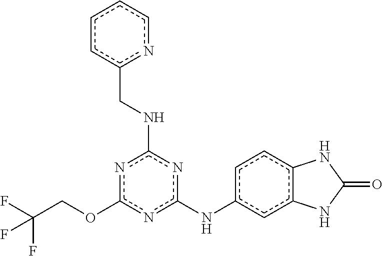 | 5-({4-[(pyridin-2- ylmethyl)amino]-6-(2,2,2- trifluoroethoxy)-1,3,5-triazin- 2-yl}amino)-1,3-dihydro-2H- benzimidazol-2-one | 432.4 | C18H15F3N8O2 |
| 17 | 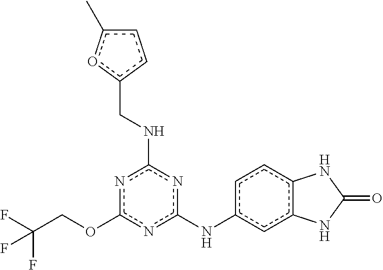 | 5-{[4-{[(5-methylfuran-2- yl)methyl]-amino}-6-(2,2,2- trifluoroethoxy)-1,3,5-triazin- 2-yl]amino}-1,3-dihydro-2H- benzimidazol-2-one | 435.4 | C18H16F3N7O3 |
| 18 | 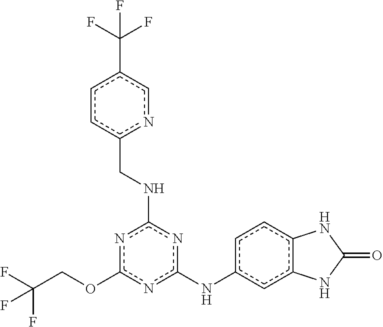 | 5-{[4-(2,2,2-trifluoroethoxy)- 6-{[4- (trifluoromethyl)benzyl]amino}- 1,3,5-triazin-2-yl]amino}- 1,3-dihydro-2H- benzimidazol-2-one | 499.4 | C20H15F6N7O2 |
| 19 | 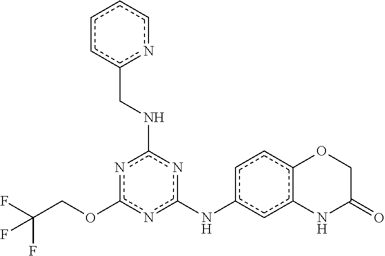 | 6-({4-[(pyridin-2- ylmethyl)amino]-6-(2,2,2- trifluoroethoxy)-1,3,5-triazin- 2-yl}amino)-2H-1,4- benzoxazin-3(4H)-one | 447.4 | C19H16F3N7O3 |
| 20 | 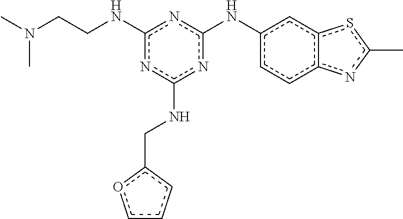 | N~2~-[2- (dimethylamino)ethyl]-N~4~- (furan-2-ylmethyl)-N~6~-(2- methyl-1,3-benzothiazol-6- yl)-1,3,5-triazine-2,4,6-triamine | 424.5 | C20H24N8OS |
| 21 | 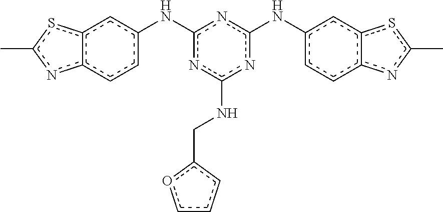 | N~2~-(furan-2-ylmethyl)- N~4~,N~6~-bis(2-methyl- 1,3-benzothiazol-6-yl)-1,3,5- triazine-2,4,6-triamine | 500.6 | C24H20N8OS2 |
| 22 | 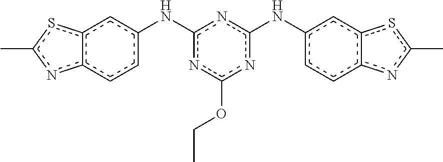 | 6-ethoxy-N,N″-bis(2-methyl- 1,3-benzothiazol-6-yl)-1,3,5- triazine-2,4-diamine | 449.6 | C21H19N7OS2 |
| 23 | 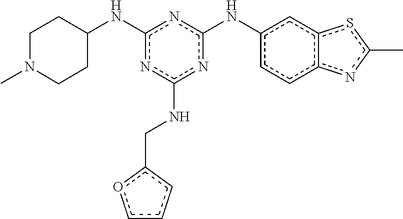 | N~2~-(furan-2-ylmethyl)- N~4~-(2-methyl-1,3- benzothiazol-6-yl)-N~6~-(1- methyl-piperidin-4-yl)-1,3,5- triazine-2,4,6-triamine | 450.6 | C22H26N8OS |
| 24 | 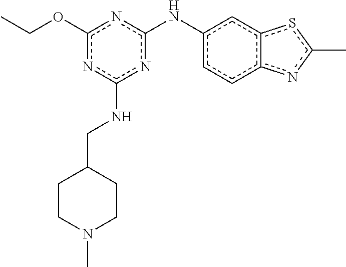 | 6-ethoxy-N-(2-methyl-1,3- benzothiazol-6-yl)-N′-[(1- methylpiperidin-4-yl)methyl]- 1,3,5-triazine-2,4-diamine | 413.5 | C20H27N7OS |
| 25 | 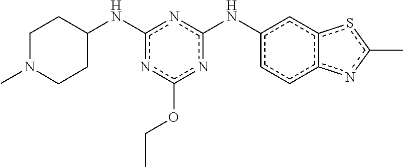 | 6-ethoxy-N-(2-methyl-1,3- benzothiazol-6-yl)-N′-(1- methylpiperidin-4-yl)-1,3,5- triazine-2,4-diamine | 399.5 | C19H25N7OS |
| 26 | 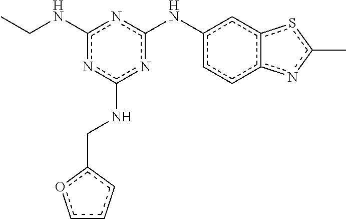 | N~2~-ethyl-N~4~-(furan-2- ylmethyl)-N~6~-(2-methyl- 1,3-benzothiazol-6-yl)-1,3,5- triazine-2,4,6-triamine | 381.5 | C18H19N7OS |
| 27 | 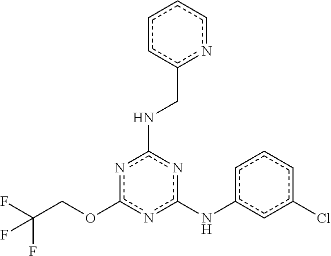 | N-(3-chlorophenyl)-N′- (pyridin-2-ylmethyl)-6-(2,2,2- trifluoroethoxy)-1,3,5- triazine-2,4-diamine | 410.8 | C17H14ClF3N6O |
| 28 | 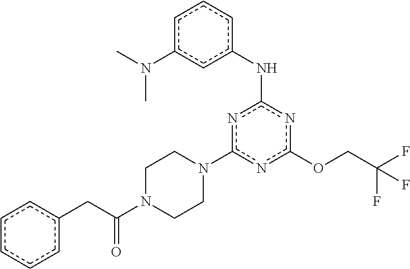 | 1-{4-[4-{[3- (dimethylamino)phenyl]- amino}-6-(2,2,2- trifluoroethoxy)-1,3,5-triazin-2- yl]piperazin-1-yl}-2- phenylethanone | 515.5 | C25H28F3N7O2 |
| 29 | 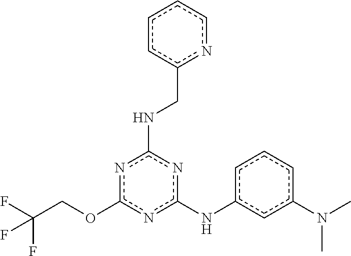 | N-[3-(dimethylamino)phenyl]- N′-(pyridin-2-ylmethyl)-6- (2,2,2-trifluoroethoxy)-1,3,5- triazine-2,4-diamine | 419.4 | C19H20F3N7O |
| 30 | Error! Objects | N-[3-(morpholin-4- | 419.2 | C20H20F3N5O2 |
| | cannot be created | ylmethyl)benzyl]-6-(2,2,2- | | |
| | from editing field | trifluoroethoxy)-N′-[3- | | |
| | codes. | (trifluoromethyl)phenyl]- | | |
| | | 1,3,5-triazine-2,4-diamine | | |
| 31 | 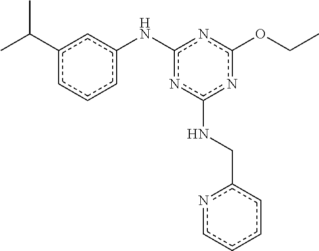 | 6-ethoxy-N-[3-(propan-2- yl)phenyl]-N′-(pyridin-2- ylmethyl)-1,3,5-triazine-2,4- diamine | 364.4 | C20H24N6O |
| 32 | 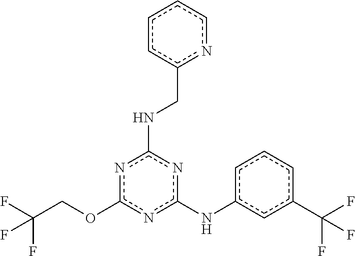 | N-(pyridin-2-ylmethyl)-6- (2,2,2-trifluoroethoxy)-N′-[3- (trifluoromethyl)-phenyl]- 1,3,5-triazine-2,4-diamine | 444.3 | C18H14F6N6O |
| 33 | 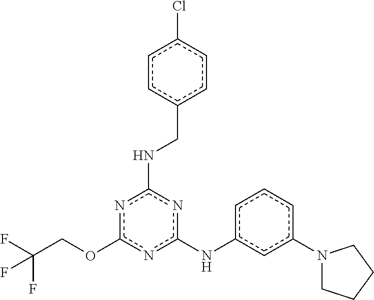 | N-(4-chlorobenzyl)-N′-[3- (pyrrolidin-1-yl)phenyl]-6- (2,2,2-trifluoroethoxy)-1,3,5- triazine-2,4-diamine | 478.9 | C22H22ClF3N6O |
| 34 | 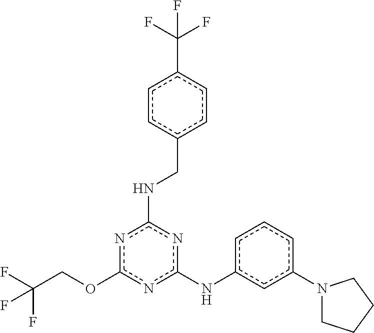 | N-[3-(pyrrolidin-1-yl)phenyl]- 6-(2,2,2-trifluoroethoxy)-N′- [4-(trifluoromethyl)-benzyl]- 1,3,5-triazine-2,4-diamine | 512.5 | C23H22F6N6O |
| 35 | 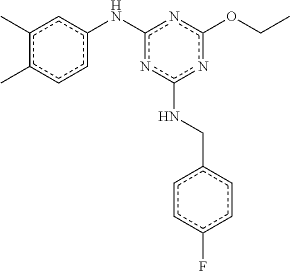 | N-(3,4-dimethylphenyl)-6- ethoxy-N′-(4-fluorobenzyl)- 1,3,5-triazine-2,4-diamine | 367.4 | C20H22FN5O |
| 36 | 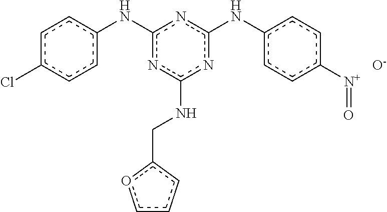 | N~2~-(4-chlorophenyl)- N~4~-(furan-2-ylmethyl)- N~6~-(4-nitrophenyl)-1,3,5- triazine-2,4,6-triamine | 437.8 | C20H16ClN7O3 |
| 37 | 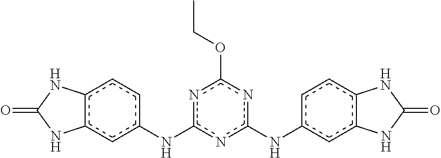 | 5,5″-[(6-ethoxy-1,3,5-triazine- 2,4-diyl)diimino]bis(1,3- dihydro-2H-benzimidazol-2-one) | 419.4 | C19H17N9O3 |
| 38 | 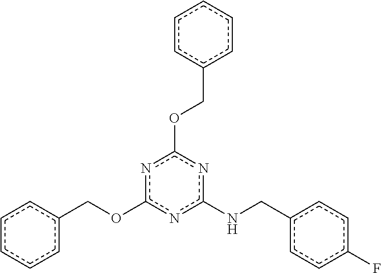 | 4,6-bis(benzyloxy)-N-(4- fluorobenzyl)-1,3,5-triazin-2- amine | 416.4 | C24H21FN4O2 |
| 39 | 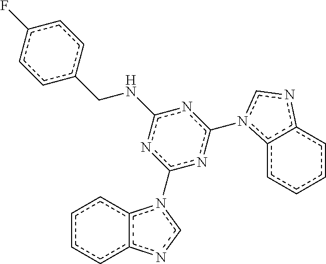 | 4,6-bis(1H-benzimidazol-1- yl)-N-(4-fluorobenzyl)-1,3,5- triazin-2-amine | 436.4 | C24H17FN8 |
| 40 | Error! Objects | 6-(2,2,2-Trifluoro-ethoxy)-N- | 353.1 | C12H9F6N5O |
| | cannot be created | (3-trifluoromethyl-phenyl)- | | |
| | from editing field | [1,3,5]triazine-2,4-diamine | | |
| | codes. | | | |
| 41 | 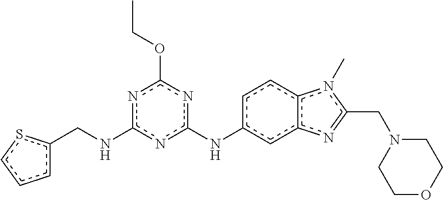 | 6-ethoxy-N-[1-methyl-2- (morpholin-4-ylmethyl)-1H- benzimidazol-5-yl]-N′- (thiophen-2-ylmethyl)-1,3,5- triazine-2,4-diamine | 480.6 | C23H28N8O2S |
| 41 | 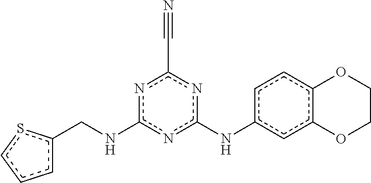 | 4-(2,3-dihydro-1,4- benzodioxin-6-ylamino)-6- [(thiophen-2- ylmethyl)amino]-1,3,5- triazine-2-carbonitrile | 366.4 | C17H14N6O2S |
| 42 | 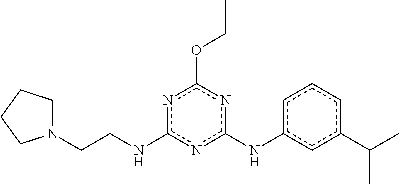 | 6-ethoxy-N-[3-(propan-2- yl)phenyl]-N′-[2-(pyrrolidin-1- yl)ethyl]-1,3,5-triazine-2,4- diamine | 370.5 | C20H30N6O |
| 43 | 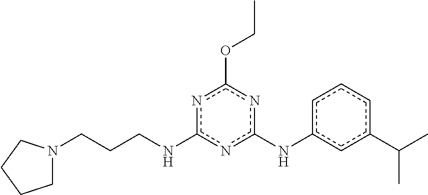 | 6-ethoxy-N-[3-(propan-2- yl)phenyl]-N′-[3-(pyrrolidin-1- yl)propyl]-1,3,5-triazine-2,4- diamine | 384.5 | C21H32N6O |
| 44 | 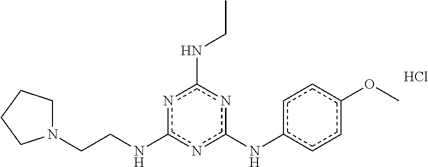 | N2-ethyl-N4-(4- methoxyphenyl)-N6-[2- (pyrrolidin-1-yl)ethyl]-1,3,5- triazine-2,4,6-triamine hydrochloride | 393.9 | C18H28ClN7O |
| 45 | 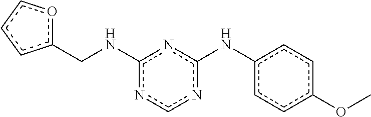 | N-(furan-2-ylmethyl)-N′-(4- methoxyphenyl)-1,3,5- triazine-2,4-diamine | 297.3 | C15H15N5O2 |
| 46 | 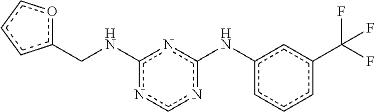 | N-(furan-2-ylmethyl)-N′-[3- (trifluoromethyl)phenyl]- 1,3,5-triazine-2,4-diamine | 335.3 | C15H12F3N5O |

### Procedures and Analytical Data for Table 43.

### 1. N-(1H-Indol-6-yl)-6-(2,2,2-trifluoro-ethoxy)-N′-(4-trifluoromethyl-benzyl)-[1,3,5]triazine-2,4-diamine

Sodium hydride (60% in oil, 60 mg, 1.5 mmol) was dissolved in a solution of 2,2,2-trifluoroethanol (150 mg, 1.5 mmol) in THF (20 mL) at 0° C. The resulting mixture was stirred at 0° C. for 10 minutes and added to a solution of N-(1H-Indol-6-yl)-6-(chloro)-N′-(4-trifluoromethyl-benzyl)-[1,3,5]triazine-2,4-diamine (420 mg, 1.0 mmol) in THF (30 mL) at 0° C. The obtained solution was stirred at 0° C. for 1 hour, at room temperature for 2 hours and at 50° C. for 1 hour. The solvent was removed at reduced pressure. The residue was diluted with water and extracted with dichloromethane. The combined organic phases were dried over sodium sulfate and concentrated. Purification by column chromatography (silica gel, 20% acetone/dichloromethane) and by recrystallization from dichloromethane/hexane gave the compound. Yield 226 mg, 50%. ¹H-NMR (400 MHz, DMSO-D₆) δ_{H}: 4.53-4.64 (2H, two broad signals, Z/E forms), 4.91 (2H, superposition of two q, J=7.5 Hz, Z/E forms), 6.32 (1H, s), 7.30-7.24 (1H, two broad d, J=8.5 Hz, Z/E forms), 7.22 (1H, s), 7.35-7.40 (1H, two broad d, J=8.5 Hz, Z/E forms), 7.53 (2H, broad, Z/E forms), 7.65 (2H, broad, Z/E forms), 7.71-7.81 (1H, broad, Z/E forms), 8.00-8.22 (1H, broad, Z/E forms), 9.39-9.50 (1H, two broad signals, Z/E forms), 10.89 (1H, broad). MW 482.39. LCMS t_{R} (min): 2.02. MS (APCI), m/z 483.15 [M+H]⁺. HPLC t_{R} (min): 15.90. M_{P} 119-121° C.

### 2. N-(4-Fluoro-benzyl)-N′-(1H-indol-6-yl)-6-(2,2,2-trifluoro-ethoxy)-[1,3,5]triazine-2,4-diamine

¹H-NMR (400 MHz, DMSO-D₆) δ_{H}: 4.41-4.59 (2H, broad, Z/E forms), 4.92 (2H, superposition of two q, J=7.5 Hz, Z/E forms), 6.32 (1H, s), 7.09 (2H, m), 7.12-7.19 (1H, broad, Z/E forms), 7.22 (1H, broad), 7.36 (3H, broad), 7.72-7.87 (1H, broad, Z/E forms), 8.02 (1H, broad), 9.30-9.50 (1H, broad, Z/E forms), 10.91 (1H, broad). MW 432.39. LCMS t_{R} (min): 1.94. MS (APCI), m/z 433.07 [M+H]⁺. HPLC t_{R} (min): 15.02. M_{P} 155-157° C.

### 3. N-(1H-Indol-6-yl)-N′-pyridin-2-ylmethyl-6-(2,2,2-trifluoro-ethoxy)-[1,3,5]triazine-2,4-diamine

¹H-NMR (400 MHz, DMSO-D₆) δ_{H}: 4.52-4.71 (2H, broad, Z/E forms), 4.80-5.01 (2H, two q, J=7.5 Hz, Z/E forms), 6.31 (1H, broad), 7.11-7.21 (1H, broad doublets, J=8.5 Hz, Z/E forms), 7.23 (2H, broad), 7.32 (1H, broad), 7.33-7.42 (1H, broad, Z/E forms), 7.71 (1H, broad), 7.77 (1H, broad), 7.98 (1H, broad), 8.50 (1H, broad), 9.32-9.52 (1H, broad, Z/E forms), 10.90 (1H, broad). MW 415.38. LCMS t_{R} (min): 1.55. MS (APCI), m/z 416.02 [M+H]⁺. HPLC t_{R} (min): 10.08. M_{P} 191-193° C.

### 4. N-(1H-Indol-6-yl)-N′-(4-methyl-benzyl)-6-(2,2,2-trifluoro-ethoxy)-[1,3,5]triazine-2,4-diamine

¹H-NMR (400 MHz, DMSO-D₆) δ_{H}: 2.25 (3H, s), 4.42-4.51 (2H, broad d, J=7.5 Hz, Z/E forms), 4.92 (2H, superposition of two broad q, J=7.5 Hz), 6.34 (1H, s), 7.10 (2H, broad), 7.22 (4H, broad), 7.40 (1H, broad), 7.71-7.88 (1H, two broad signals, Z/E forms), 7.96 (1H, broad, Z/E forms), 9.34-9.48 (1H, two broad signals, Z/E forms), 10.89 (1H, broad). MW 428.42. LCMS t_{R} (min): 1.99. MS (APCI), m/z 429.04 [M+H]⁺. HPLC t_{R} (min): 15.41. M_{P} 113-115° C.

### 5. N-(1H-Indol-6-yl)-N′-(5-methyl-isoxazol-3-ylmethyl)-6-(2,2,2-trifluoro-ethoxy)-[1,3,5]triazine-2,4-diamine

¹H-NMR (400 MHz, DMSO-D₆) δ_{H}: 2.32 (3H, s), 4.40-4.63 (2H, broad, Z/E forms), 4.92 (2H, superposition of two broad q, J=7.5 Hz), 6.12 (1H, broad), 6.32 (1H, broad), 7.20 (2H, broad), 7.40 (1H, broad), 7.62-7.89 (1H, broad, Z/E forms), 7.96 (1H, broad), 9.35-9.60 (1H, broad, Z/E forms), 10.89 (1H, broad). MW 419.37. LCMS t_{R} (min): 1.85. MS (APCI), m/z 420.15 [M+H]⁺. HPLC t_{R} (min): 13.59. M_{P} 205-207° C.

### 6. 6-Ethoxy-N-(1H-indol-6-yl)-N′-pyridin-4-ylmethyl-[1,3,5]triazine-2,4-diamine

DIPEA (516 mg, 4.0 mmol) was added dropwise at 0° C. to a mixture of 2,4-dichloro-6-ethoxy-1,3,5-triazine (580 mg, 4.0 mmol), 1H-Indol-6-ylamine (528 mg, 4.0 mmol) and THF (10 mL). The obtained mixture was stirred at room temperature 1 hours (TLC control). The obtained mixture was transferred onto a column with silica gel without work up and purified (20% ethyl acetate/hexane) giving compound 4-Chloro-6-ethoxy-[1,3,5]triazin-2-yl)-(1H-indol-6-yl)-amine Yield 587 mg, 50%.

A mixture of 4-Chloro-6-ethoxy-[1,3,5]triazin-2-yl)-(1H-indol-6-yl)-amine (580 mg, 2 mmol), C-Pyridin-4-yl-methylamine (216 mg, 2 mmol), K₂CO₃ (560 mg, 4.0 mmol) and DMSO (2 mL) was stirred at 90° C. for 5 hours, cooled to room temperature. The formed solid was collected by filtration and purified by column chromatography (silica gel, 20% ethyl acetate/hexane) furnishing a final compound. Yield 73 mg, 10%. ¹H-NMR (400 MHz, DMSO-D₆) δ_{H}: 1.18-1.38 (3H, broad, Z/E forms), 4.20-4.40 (2H, broad, Z/E forms), 4.48-4.61 (2H, broad, Z/E forms), 6.33 (1H, s), 7.10-7.18 (1H, broad), 7.22 (1H, s), 7.32 (3H, broad), 7.82 (2H, broad, Z/E forms), 8.49 (2H, broad), 9.18-9.28 (1H, broad, Z/E forms), 10.88 (1H, broad). LCMS t_{R} (min): 1.37. MS (APCI), m/z 361.94 [M+H]⁺. HPLC t_{R} (min): 8.13. Mₚ 198-200° C.

### 7. N-(1H-Indazol-6-yl)-6-(2,2,2-trifluoro-ethoxy)-N′-(4-trifluoromethyl-benzyl)-[1,3,5]triazine-2,4-diamine

¹H-NMR (400 MHz, DMSO-D₆) δ_{H}: 4.56-4.63 (2H, broad, Z/E forms), 4.95 (2H, superposition of two broad q, J=7.5 Hz, Z/E forms), 7.24-7.37 (1H, two broad doublets, J=8.5 Hz, Z/E forms), 7.56 (2H, d, J=8.5 Hz), 7.64 (2H, broad), 7.66 (1H, broad), 7.92 (1H, s), 7.98-8.02 (1H, two s, Z/E forms), 8.30 (1H, broad), 9.64-9.78 (1H, two broad signals, Z/E forms), 12.80 (1H, broad). MW 483.38. LCMS t_{R} (min): 1.89. MS (APCI), m/z 484.13 [M+H]⁺. HPLC t_{R} (min): 14.67. M_{P} 230-232° C.

### 8. N-(4-Chloro-benzyl)-N′-(1H-indol-6-yl)-6-(2,2,2-trifluoro-ethoxy)-[1,3,5]-triazine-2,4-diamine

¹H-NMR (400 MHz, DMSO-D₆) δ_{H}: 4.41-4.59 (2H, broad, Z/E forms), 4.92 (2H, superposition of two q, J=7.5 Hz), 6.35 (1H, s), 7.18 (1H, broad), 7.22 (1H, broad), 7.38 (5H, broad), 7.67-7.89 (1H, broad, Z/E forms), 8.05 (1H, broad), 9.32-9.55 (1H, broad, Z/E forms), 10.90 (1H, broad). MW 448.84. LCMS t_{R} (min): 1.99. MS (APCI), m/z 449.04, 451.08 [M+H]⁺. HPLC t_{R} (min): 15.63. M_{P} 192-194° C.

### 9. 4-{[4-(1H-Indol-6-ylamino)-6-(2,2,2-trifluoro-ethoxy)-[1,3,5]triazin-2-ylamino]-methyl}-benzonitrile

¹H-NMR (400 MHz, DMSO-D₆) δ_{H}: 4.52-4.68 (2H, broad, Z/E forms), 4.90 (2H, superposition of two q, Z/E forms), 6.32 (1H, s), 7.08-7.28 (1H, two broad d, J=8.5 Hz, Z/E forms), 7.22 (1H, s), 7.32-7.43 (1H, two d, J=8.5 Hz, Z/E forms), 7.50 (2H, broad), 7.75 (3H, broad), 8.00-8.20 (1H, broad, Z/E forms), 9.35-9.52 (1H, broad, Z/E forms), 10.90 (1H, broad). MW 439.40. LCMS t_{R} (min): 1.84. MS (APCI), m/z 440.09 [M+H]⁺. HPLC t_{R} (min): 14.29. M_{P} 104-106° C.

### 10. 6-Ethoxy-N,N′-bis-(1H-indol-6-yl)-[1,3,5]triazine-2,4-diamine

¹H-NMR (400 MHz, DMSO-D₆) δ_{H}: 1.35 (3H, t, J=7.5 Hz), 4.40 (2H, q, J=7.5 Hz), 6.36 (2H, broad), 7.22 (2H, s), 7.32 (2H, broad, 7.42 (2H, d, J=8.5 Hz), 7.81 (2H, broad), 9.26 (2H, broad), 10.86 (2H, broad). LCMS t_{R} (min): 1.77. MS (APCI), m/z 386.04 [M+H]⁺. HPLC t_{R} (min): 12.12. Mₚ 156-158° C.

### 11. 5-[4-(4-Fluoro-benzylamino)-6-(2,2,2-trifluoro-ethoxy)-[1,3,5]triazin-2-ylamino]-1,3-dihydro-benzoimidazol-2-one

¹H-NMR (400 MHz, DMSO-D₆) δ_{H}: 4.50 (2H, broad), 4.90 (2H, superposition of two broad q, J=7.5 Hz, Z/E forms), 6.80 (1H, broad), 7.10 (2H, broad), 7.10-7.22 (1H, two broad signals, Z/E forms), 7.34 (2H, broad), 7.34-7.39 (1H, two broad signals, Z/E forms), 7.98-8.04 (1H, two broad signals, Z/E forms), 9.29-9.43 (1H, two broad signals, Z/E forms), 10.32 (1H, broad), 10.42-10.49 (1H, two broad signals, Z/E forms). MW 449.37. LCMS t_{R} (min): 1.68. MS (APCI), m/z 450.18 [M+H]⁺. HPLC t_{R} (min): 12.00. M_{P} 142-144° C.

### 12. 5-[4-(4-Chloro-benzylamino)-6-(2,2,2-trifluoro-ethoxy)-[1,3,5]triazin-2-ylamino]-1,3-dihydro-benzoimidazol-2-one

¹H-NMR (400 MHz, DMSO-D₆) δ_{H}: 4.50 (2H, broad), 4.90 (2H, broad), 6.80 (1H, broad), 7.07-7.25 (1H, two d, J=8.5 Hz), 7.45 (5H, broad), 7.93-8.19 (1H, broad, Z/E forms), 9.28-9.50 (1H, broad, Z/E forms), 10.30-10.52 (2H, broad, Z/E forms). MW 465.83. LCMS t_{R} (min): 1.74. MS (APCI), m/z 466.13 [M+H]⁺. HPLC t_{R} (min): 12.06. M_{P} 255-257° C.

### 13. 5-[4-Benzylamino-6-(2,2,2-trifluoro-ethoxy)-[1,3,5]triazin-2-ylamino]-1,3-dihydro-benzoimidazol-2-one

¹H-NMR (400 MHz, DMSO-D₆) δ_{H}: 4.50 (2H, broad), 4.90 (2H, broad), 6.78 (1H, broad), 7.10-7.40 (7H, m), 8.02 (1H, broad), 9.25-9.48 (1H, broad, Z/E forms), 10.34 (1H, broad), 10.47 (1H, broad). MW 431.38. LCMS t_{R} (min): 1.65. MS (APCI), m/z 432.06 [M+H]⁺. HPLC t_{R} (min): 11.77. M_{P} 280-282° C.

### 14. 5-[4-(Cyclohexylmethyl-amino)-6-(2,2,2-trifluoro-ethoxy)-[1,3,5]triazin-2-ylamino]-1,3-dihydro-benzoimidazol-2-one

¹H-NMR (400 MHz, DMSO-D₆) δ_{H}: 0.90 (2H, m), 1.16 (3H, m), 1.58 (2H, m), 1.68 (4H, m), 3.12 (2H, broad), 4.90 (2H, superposition of two q, J=7.5 Hz), 6.77 (1H, d, J=8.5 Hz), 7.22 (1H, broad), 7.36 (1H, broad), 7.40-7.58 (1H, broad, Z/E forms), 9.13-9.40 (1H, broad, Z/E forms), 10.33 (1H, broad), 10.40-10.51 (1H, broad, Z/E forms). MW 437.43. LCMS t_{R} (min): 1.78. MS (APCI), m/z 438.11 [M+H]⁺. HPLC t_{R} (min): 13.01. M_{P} 300-302° C.

### 15. 5-{4-Ethoxy-6-[(pyridin-4-ylmethyl)-amino]-[1,3,5]triazin-2-ylamino}-1,3-dihydro-benzoimidazol-2-one

DIPEA (258 mg, 2.0 mmol) was added dropwise at room temperature to a mixture of 2,4-dichloro-6-ethoxy-1,3,5-triazine (288 mg, 2.0 mmol), 5-amino-1,3-dihydro-benzoimidazol-2-one (298 mg, 2.0 mmol) and THF (20 mL). The obtained mixture was stirred at room temperature up to 3 hours (TLC control). The obtained mixture was washed by water. The organic layer was concentrated and recrystallizad from DMSO/ether/hexane (1/4/4) giving compound 5-(4-Chloro-6-ethoxy-[1,3,5]triazin-2-ylamino)-1,3-dihydro-benzoimidazol-2-one. Yield 570 mg, 92%.

A mixture of compound 5-(4-Chloro-6-ethoxy-[1,3,5]triazin-2-ylamino)-1,3-dihydro-benzoimidazol-2-one (307 mg, 1 mmol), C-Pyridin-4-yl-methylamine (108 mg, 1 mmol), DIPEA (162 mg, 1.5 mmol) and DMSO (1 mL) was stirred at 60° C. for 45 minutes, cooled to room temperature and transferred to column without work up for purification by column chromatography (silica gel, 15% MeOH/ethyl acetate), then compound was recrystallized from DMSO/MeOH and purified by prepTLC (25% MeOH/ethyl acetate) furnishing a final compound.

Yield 64 mg, 17%. ¹H-NMR (400 MHz, DMSO-D₆) δ_{H}: 1.12-1.34 (3H, broad, Z/E forms), 4.15-4.35 (2H, broad, Z/E forms), 4.51 (2H, broad), 6.70-6.85 (1H, broad, Z/E forms), 7.02-7.12 (1H, broad, Z/E forms), 7.32 (2H, broad), 7.39-7.48 (1H, broad, Z/E forms), 7.74-7.95 (1H, broad, Z/E forms), 8.50 (2H, broad), 9.13-9.28 (1H, broad, Z/E forms), 10.33 (1H, s), 10.45 (1H, s). LCMS t_{R} (min): 1.20. MS (APCI), m/z 379.05 [M+H]⁺. HPLC t_{R} (min): 5.99. Mₚ 210-212° C.

### 16. 5-[4-[(Pyridin-2-ylmethyl)-amino]-6-(2,2,2-trifluoro-ethoxy)-[1,3,5]triazin-2-ylamino]-1,3-dihydro-benzoimidazol-2-one

Yield 290 mg, 68%. ¹H-NMR (400 MHz, DMSO-D₆) δ_{H}: 4.55-4.70 (2H, broad), 4.79-5.00 (2H, two q, J=7.5 Hz, Z/E forms), 6.77-6.85 (1H, broad, Z/E forms), 7.06-7.25 (1H, broad, Z/E forms), 7.19-7.28 (1H, broad, Z/E forms), 7.30 (2H, broad), 7.70 (1H, broad), 7.98 (1H, broad), 8.50 (1H, s), 9.30-9.47 (1H, broad, Z/E forms), 10.28-10.50 (2H, broad). LCMS t_{R} (min): 1.27. MS (APCI), m/z 433.22 [M+H]⁺. HPLC t_{R} (min): 7.66. M_{P} 325-327° C.

### 17. 5-[4-[(5-Methyl-furan-2-ylmethyl)-amino]-6-(2,2,2-trifluoro-ethoxy)-[1,3,5]triazin-2-ylamino]-1,3-dihydro-benzoimidazol-2-one

To a solution of compound 2,4-dichloro-6-[(5-methyl-furan-2-ylmethyl)-amino]-[1,3,5]triazin (777 mg, 3.0 mmol) in DMSO (2.5 mL) a solution of 5-amino-1,3-dihydro-benzoimidazol-2-one (447 mg, 3.0 mmol) and DIPEA (387 mg, 3.0 mmol) in DMSO (2.5 mL) was added at room temperature. The obtained mixture was stirred at room temperature for 2 hours, diluted with water. The formed solid was collected by filtration and purified by column chromatography (silica gel ethyl acetate) to give 5-{4-Chloro-6-[(5-methyl-furan-2-ylmethyl)-amino]-[1,3,5]triazin-2-ylamino}-1,3-dihydro-benzoimidazol-2-one. Yield 600 mg, 53%.

Sodium hydride (60% in oil, 117 mg 3.0 mmol) was added slowly to a solution of 2,2,2-trifluoroethanol (260 mg, 2.6 mmol) in DMF (5 mL) at 0° C. The obtained mixture was allowed to warm up to room temperature. 5-{4-Chloro-6-[(5-methyl-furan-2-ylmethyl)-amino]-[1,3,5]triazin-2-ylamino}-1,3-dihydro-benzoimidazol-2-one (500 mg, 1.3 mmol) was added to the mixture at room temperature and the resulting mixture was stirred at room temperature for 4 hours, diluted with water. The formed solid was collected by filtration and purified by recrystallization from ethanol, prepTLC (10% MeOH/ethyl acetate), recrystallization from ethanol and from MeOH/ether giving a final compound. Yield 93 mg, 16%. ¹H-NMR (400 MHz, DMSO-D₆) δ_{H}: 2.22 (3H, s), 4.42 (2H, broad), 4.93 (2H, broad), 5.96 (1H, broad), 6.12 (1H, broad), 6.80 (1H, d, J=8.5 Hz), 7.20-7.45 (1H, broad, Z/E forms), 7.22 (1H, broad), 7.89 (1H, broad), 9.29-9.51 (1H, broad, Z/E forms), 10.28-10.53 (2H, broad, Z/E forms). LCMS t_{R} (min): 1.66. MS (APCI), m/z 436.04 [M+H]⁺. HPLC t_{R} (min): 11.72. Mₚ 164-166° C.

### 18. 5-[4-(2,2,2-Trifluoro-ethoxy)-6-(4-trifluoromethyl-benzylamino)-[1,3,5]triazin-2-ylamino]-1,3-dihydro-benzoimidazol-2-one

¹H-NMR (400 MHz, DMSO-D₆) δ_{H}: 4.60 (2H, broad), 4.80-5.00 (2H, two q, J=7.5 Hz, Z/E forms), 6.70-6.86 (1H, two d, J=8.5 Hz, Z/E forms), 7.02-7.28 (1H, two d, J=8.5 Hz, Z/E forms), 7.15-7.40 (1H, broad, Z/E forms), 7.51 (2H, broad d, J=8.5 Hz), 7.67 (2H, broad d, J=8.5 Hz), 8.02-8.22 (1H, broad, Z/E forms), 9.30-9.50 (1H, broad, Z/E forms), 10.36 (1H, broad), 10.40-10.50 (1H, broad, Z/E forms). LCMS t_{R} (min): 1.77. MS (APCI), m/z 500.21 [M+H]⁺. HPLC t_{R} (min): 12.99. M_{P} 271-273° C.

### 19. 6-[4-[(Pyridin-2-ylmethyl)-amino]-6-(2,2,2-trifluoro-ethoxy)-[1,3,5]triazin-2-ylamino]-4H-benzo[1,4]oxazin-3-one

¹H-NMR (400 MHz, DMSO-D₆) δ_{H}: 4.48 (2H, s), 4.57-4.67 (2H, two broad signals, Z/E forms), 4.80-5.00 (2H, two q, J=7.5 Hz, Z/E forms), 6.76-6.82 (1H, broad d, J=8.5 Hz, Z/E forms), 7.01-7.22 (1H, two d, J=8.5 Hz, Z/E forms), 7.28 (1H, broad, Z/E forms), 7.31 (2H, broad, Z/E forms), 7.72 (1H, t, J=8.0 Hz), 7.91-8.07 (1H, two broad signals, Z/E forms), 8.49 (1H, broad, Z/E forms), 9.47-9.55 (1H, two broad signals, Z/E forms), 10.63 (1H, broad, Z/E forms). MW 447.38. LCMS t_{R} (min): 1.43. MS (APCI), m/z 448.14 [M+H]⁺. HPLC t_{R} (min): 8.79. M_{P} 141-143° C.

### 19. N-(2-Dimethylamino-ethyl)-N′-furan-2-ylmethyl-N″-(2-methyl-benzothiazol-6-yl)-[1,3,5]triazine-2,4,6-triamine

¹H-NMR (400 MHz, DMSO-D₆) δ_{H}: 2.19 (6H, s), 2.41 (2H, t, J=7.5 Hz), 2.72 (3H, s), 3.38 (2H, broad), 4.49 (2H, d, J=7.5 Hz), 6.23 (1H, broad), 6.38 (1H, broad), 6.60 (1H, broad), 7.18 (1H, broad), 7.52 (1H, s), 7.64 (1H, d, J=8.5 Hz), 7.71 (1H, d, J=8.5 Hz), 8.67 (1H, broad), 9.04 (1H, broad). LCMS t_{R} (min): 1.44. MS (APCI), m/z 425.11 [M+H]⁺. HPLC t_{R} (min): 8.24. Mₚ 92-94° C.

### 21. N-Furan-2-ylmethyl-N′,N″-bis-(2-methyl-benzothiazol-6-yl)-[1,3,5]triazine-2,4,6-triamine

¹H-NMR (400 MHz, DMSO-D₆) δ_{H}: 2.74 (6H, s), 4.55 (2H, d, J=7.5 Hz), 6.29 (1H, broad), 6.39 (1H, broad), 7.58 (2H, broad), 7.68 (2H, d, J=8.5 Hz), 7.77 (2H, d, J=8.5 Hz), 8.60 (2H, broad), 9.28 (2H, broad). LCMS t_{R} 1.64 (min). MS (APCI), m/z 501.01 [M+H]⁺. Mₚ 165-167° C.

### 22. 6-Ethoxy-N,N′-bis-(2-methyl-benzothiazol-6-yl)-[1,3,5]triazine-2,4-diamine

¹H-NMR (400 MHz, DMSO-D₆) δ_{H}: 1.32 (3H, t, J=7.5 Hz), 2.78 (6H, s), 4.40 (2H, q, J=7.5 Hz), 7.70 (2H, d, J=8.5 Hz), 7.82 (2H, d, J=8.5 Hz), 8.46 (2H, broad), 9.80 (2H, broad). LCMS t_{R} 1.76 (min). MS (APCI), m/z 449.98 [M+H]⁺. Mₚ 142-144° C.

### 23. N-Furan-2-ylmethyl-N′-(2-methyl-benzothiazol-6-yl)-N″-(1-methyl-piperidin-4-yl)-[1,3,5]triazine-2,4,6-triamine

¹H-NMR (400 MHz, DMSO-D₆) δ_{H}: 1.62 (2H, m), 1.88 (2H, m), 2.20-2.40 (5H, m), 2.72 (3H, s), 2.94 (2H, broad), 3.81 (1H, broad), 4.48 (2H, d, J=7.5 Hz), 6.22 (1H, dd, J=3.6 1.8 Hz), 6.38 (1H, d, J=3.6 Hz), 6.78 (1H, broad), 7.12 (1H, broad, Z/E forms), 7.52 (1H, d, J=1.8 Hz), 7.62 (1H, d, J=5.0 Hz), 7.71 (1H, d, J=8.5 Hz), 8.66 (1H, broad), 8.80-9.20 (1H, broad, Z/E forms). LCMS t_{R} 1.37 (min). MS (APCI), m/z 451.06 [M+H]⁺. Mₚ 62-65° C.

### 24. 6-Ethoxy-N-(2-methyl-benzothiazol-6-yl)-N′-(1-methyl-piperidin-4-ylmethyl)-[1,3,5]triazine-2,4-diamine

¹H-NMR (400 MHz, DMSO-D₆) δ_{H}: 1.18 (2H, m), 1.28 (3H, broad), 1.40-1.70 (3H, m), 1.80 (2H, m), 2.11 (3H, s), 2.74 (5H, m), 3.30 (2H, m), 4.30 (2H, broad), 7.42-7.53 (1H, broad, Z/E forms), 7.65 (1H, d, J=8.5 Hz), 7.76 (1H, d, J=8.5 Hz), 8.58 (1H, broad), 9.36-9.59 (1H, broad, Z/E forms). LCMS t_{R} (min): 1.38. MS (APCI), m/z 414.17 [M+H]⁺. HPLC t_{R} (min): 8.25. Mₚ 203-205° C.

### 25. 6-Ethoxy-N-(2-methyl-benzothiazol-6-yl)-N′-(1-methyl-piperidin-4-yl)-[1,3,5]triazine-2,4-diamine

¹H-NMR (400 MHz, DMSO-D₆) δ_{H}: 1.28 (3H, t, J=7.5 Hz), 1.54 (2H, m), 1.87 (2H, m), 2.06 (2H, m), 2.11 (3 h, m), 2.20 (3H, broad), 2.81, (2H, broad), 3.75 (1H, m, broad), 4.25-4.45 (2H, broad, Z/E forms), 7.34 (1H, broad), 7.63 (1H, broad), 7.78 (1H, d, J=8.5 Hz), 8.60 (1H, broad), 9.35-9.60 (1H, broad, Z/E forms). LCMS t_{R} (min): 1.33. MS (APCI), m/z 400.01 [M+H]⁺. HPLC t_{R} (min): 8.33. Mₚ 113-115° C.

### 26. N-Ethyl-N′-furan-2-ylmethyl-N″-(2-methyl-benzothiazol-6-yl)-[1,3,5]triazine-2,4,6-triamine

¹H-NMR (400 MHz, DMSO-D₆) δ_{H}: 1.10 (3H, t, J=7.5 Hz), 2.73 (3H, s), 3.28 (2H, q, J=7.5 Hz), 4.48 (2H, d, J=7.5 Hz), 6.22 (1H, broad), 6.36 (1H, broad), 6.77 (1H, broad), 7.18 (1H, broad), 7.53 (1H, s), 7.67 (1H, d, J=8.5 Hz, broad), 7.72 (1H, d, J=8.5 Hz), 8.60-8.75 (1H, broad, Z/E forms), 8.80-9.15 (1H, broad, Z/E forms). LCMS t_{R} (min): 1.52. MS (APCI), m/z [M+H]⁺ 381.99. HPLC t_{R} (min): 10.40. Mₚ 97-99° C.

### 27. N-(3-Chloro-phenyl)-N′-pyridin-2-ylmethyl-6-(2,2,2-trifluoro-ethoxy)-[1,3,5]triazine-2,4-diamine

¹H-NMR (400 MHz, DMSO-D₆) δ_{H}: 4.61 (2H, broad), 4.81-5.02 (2H, two q, J=7.5 Hz, Z/E forms), 6.95-7.05 (1H, two d, J=8.5 Hz, Z/E forms), 7.23 (2H, broad), 7.31 (1H, d, J=8.5 Hz), 7.45-7.68 (1H, two d, J=8.5 Hz, Z/E forms), 7.72 (1H, m), 7.73-7.94 (1H, broad, Z/E forms), 8.25 (1H, broad), 8.49 (1H, broad), 9.69-9.80 (1H, broad, Z/E forms). MW 410.79. LCMS t_{R} (min): 1.68. MS (APCI), m/z 411.00, 413.00 [M+H]⁺. HPLC t_{R} (min): 11.06. M_{P} 67-69° C.

### 28. 1-{4-[4-(3-Dimethylamino-phenylamino)-6-(2,2,2-trifluoro-ethoxy)-[1,3,5]triazin-2-yl]-piperazin-1-yl}-2-phenyl-ethanone

¹H-NMR (400 MHz, DMSO-D₆) δ_{H}: 2.90 (6H, s), 3.58 (4H, broad), 3.65-3.75 (4H, broad), 3.79 (2H, s), 4.98 (2H, q, J=7.5 Hz), 6.40 (1H, d, J=8.5 Hz), 6.89 (1H, broad d, J=8.5 Hz), 7.07 (1H, t, J=8.5 Hz), 7.15-7.22 (1H, broad, Z/E forms), 7.24 (3H, m), 7.30 (2H, d, J=8.5 Hz), 9.48 (1H, broad). MW 515.54. LCMS t_{R} (min): 1.85. MS (APCI), m/z 516.24 [M+H]⁺. HPLC t_{R} (min): 11.60. M_{P} 184-186° C.

### 29. N-(3-Dimethylamino-phenyl)-N′-pyridin-2-ylmethyl-6-(2,2,2-trifluoro-ethoxy)-[1,3,5]triazine-2,4-diamine

¹H-NMR (400 MHz, DMSO-D₆) δ_{H}: 2.70-2.92 (6H, two s, Z/E forms), 4.52-4.70 (2H, broad, Z/E forms), 4.80-5.00 (2H, two q, J=7.5 Hz, Z/E forms), 6.30-6.45 (1H, broad, Z/E forms), 6.80-7.00 (1H, broad, Z/E forms), 7.05 (1H, broad), 7.12 (1H, broad), 7.22 (1H, d/d, J=8.0/5.0 Hz), 7.29 (1H, d, J=8.0 Hz), 7.72 (1H, t, J=8.0 Hz), 8.06 (1H, broad), 8.49 (1H, broad), 9.28-9.40 (1H, broad, Z/E forms). MW 419.41. LCMS t_{R} (min): 1.40. MS (APCI), m/z 420.23 [M+H]⁺. HPLC t_{R} (min): 7.55. M_{P} 75-77° C.

### 30. N-(3-Isopropyl-phenyl)-N′-pyridin-2-ylmethyl-6-(2,2,2-trifluoro-ethoxy)-[1,3,5]triazine-2,4-diamine

To a solution of 4,6-diChloro-N-(3-isopropyl-phenyl)-[1,3,5]triazine-2-diamine (400 mg, 1.41 mmol) in acetonitrile (7 mL) C-pyridin-2-yl-methylamine (153 mg, 1.41 mmol) and NEt₃ (214 mg, 2.11 mmol) were added dropwise at room temperature. The resulting mixture was stirred at room temperature for 3 hours and diluted with water. The residue was washed with water and hexane and dried giving 6-Chloro-N-(3-isopropyl-phenyl)-N′-pyridin-2-ylmethyl-[1,3,5]triazine-2,4-diamine. Yield 454 g, 91%.

A mixture of 6-Chloro-N-(3-isopropyl-phenyl)-N′-pyridin-2-ylmethyl-[1,3,5]triazine-2,4-diamine (454 mg, 1.28 mmol), 2,2,2-trifluoro-ethanol (384 mg, 3.84 mmol), K₂CO₃ (353 mg, 2.56 mmol) and DMSO (4 mL) was stirred at 100° C. for 4 hours, cooled to room temperature and diluted with water. The formed solid was collected by filtration and purified by prepTLC (20% ethanol/chloroform) giving the compound. Yield 320 mg, 60%. ¹H-NMR (400 MHz, DMSO-D₆) δ_{H}: 1.10-1.22 (6H, broad, Z/E forms), 2.73-2.88 (1H, broad, Z/E forms), 4.60-4.70 (2H, broad, Z/E forms), 4.88-5.00 (2H, two broad q, J=7.5 Hz, Z/E forms), 6.83-6.89 (1H, two broad peaks, Z/E forms), 7.10-7.19 (1H, two broad peaks, Z/E forms), 7.23 (2H, broad), 7.30-7.52 (1H, broad, Z/E forms), 7.57 (1H, broad), 7.73 (1H, broad t, J=8.5 Hz), 8.09 (1H, broad), 8.51 (1H, broad), 9.42-9.49 (1H, broad). MW 418.42. LCMS t_{R} (min): 1.88. MS (APCI), m/z 419.10 [M+H]⁺. HPLC t_{R} (min): 12.15. M_{P} 130-132° C.

### 31. 6-Ethoxy-N-(3-isopropyl-phenyl)-N′-pyridin-2-ylmethyl-[1,3,5]triazine-2,4-diamine

¹H-NMR (400 MHz, DMSO-D₆) δ_{H}: 1.05-1.28 (6H, broad, Z/E forms), 1.28-1.38 (3H, broad peaks, Z/E forms), 2.78 (1H, broad peak, Z/E forms), 4.20-4.39 (2H, broad peak, Z/E forms), 4.55-4.70 (2H, broad peak, Z/E forms), 6.82 (1H, broad peak), 7.05-7.19 (1H, broad peak, Z/E forms), 7.23 (1H, broad peak), 7.30 (2H, m), 7.60 (1H, broad peak), 7.72 (1H, t, J=8.0 Hz), 7.72-7.82 (1H, broad peaks, Z/E forms), 8.50 (1H, broad peak), 9.15-9.30 (1H, broad peak, Z/E forms). MW 364.45. LCMS t_{R} (min): 1.70. MS (APCI), m/z 365.12 [M+H]⁺. HPLC t_{R} (min): 10.99. M_{P} 125-127° C.

### 32. N-Pyridin-2-ylmethyl-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 4.62 (2H, broad), 4.82-5.02 (2H, two q, J=7.5 Hz, Z/E forms), 721 (1H, broad), 7.31 (2H, broad), 7.40-7.58 (1H, two broad triplets, J=8.5 Hz, Z/E forms), 7.72 (1H, broad), 7.75-8.06 (1H, broad, Z/E forms), 8.12 (1H, broad), 8.23 (1H, broad), 8.50 (1H, broad), 9.91 (1H, broad). LCMS t_{R} (min): 1.77. MS (APCI), m/z 445.13 [M+H]⁺. HPLC t_{R} (min): 11.70. M_{P} 191-193° C.

### 33. N-(4-Chloro-benzyl)-N′-(3-pyrrolidin-1-yl-phenyl)-6-(2,2,2-trifluoro-ethoxy)-[1,3,5]triazine-2,4-diamine

¹H-NMR (400 MHz, DMSO-D₆) δ_{H}: 1.85-1.93 (4H, two broad signals, Z/E forms), 3.05-3.21 (4H, two broad signals, Z/E forms), 4.49-4.58 (2H, two broad signals, Z/E forms), 4.95 (2H, superposition of two broad q, J=7.5 Hz, Z/E forms), 6.21 (1H, broad, Z/E forms), 6.79-6.95 (1H, to broad signals, Z/E forms), 7.03 (2H, broad, Z/E forms), 7.31-7.37 (4H, broad, Z/E forms), 8.05-8.13 (1H, two broad signals, Z/E forms), 9.30-9.40 (1H, two broad signals, Z/E forms).

### 34. N-(3-Pyrrolidin-1-yl-phenyl)-6-(2,2,2-trifluoro-ethoxy)-N′-(4-trifluoromethyl-benzyl)-[1,3,5]triazine-2,4-diamine

¹H-NMR (400 MHz, DMSO-D₆) δ_{H}: 1.82-1.95 (4H, broad, Z/E forms), 3.05-3.21 (4H, broad, Z/E forms), 4.60-4.65 (2H, broad, Z/E forms), 4.85-5.00 (2H, two q, J=7.5 Hz, Z/E forms), 6.20 (1H, superposition of two d, J=8.5 Hz, Z/E forms), 6.72-6.95 (1H, broad, Z/E forms), 6.97 (1H, d, J=8.5 Hz), 7.00 (1H, broad), 7.50 (2H, broad), 7.66 (2H, broad d, J=8.5 Hz), 8.11-8.20 (1H, broad, Z/E forms), 9.28-9.40 (1H, broad, Z/E forms). MW 512.46. LCMS t_{R} (min): 2.30. MS (APCI), m/z 513.21 [M+H]⁺. HPLC t_{R} (min): 16.97. Mₚ 182-184° C.

### 35. N-Pyridin-2-ylmethyl-6-(2,2,2-trifluoro-ethoxy)-N′-(3-trifluoromethyl-phenyl)-[1,3,5]triazine-2,4-diamine

¹H-NMR (400 MHz, DMSO-D₆) δ_{H}: 4.62 (2H, broad), 4.82-5.02 (2H, two q, J=7.5 Hz, Z/E forms), 7.21 (1H, broad), 7.31 (2H, broad), 7.40-7.58 (1H, two broad triplets, J=8.5 Hz, Z/E forms), 7.72 (1H, broad), 7.75-8.06 (1H, broad, Z/E forms), 8.12 (1H, broad), 8.23 (1H, broad), 8.50 (1H, broad), 9.91 (1H, broad). LCMS t_{R} (min): 1.77. MS (APCI), m/z 445.13 [M+H]⁺. HPLC t_{R} (min): 11.70. M_{P} 191-193° C.

### 36. N-(4-Chloro-phenyl)-N′-furan-2-ylmethyl-N″-(4-nitro-phenyl)-[1,3,5]triazine-2,4,6-triamine

¹H-NMR (400 MHz, DMSO-D₆) δ_{H}: 4.56 (2H, d, J=7.5 Hz), 6.30 (1H, dd, J=3.6, 1.8 Hz), 6.40 (1H, broad), 7.32 (2H, d, J=8.5 Hz), 7.57 (1H, s), 7.70-7.90 (3H, m, broad), 8.80-8.20 (4H, m), 9.20-9.40 (1H, broad, Z/E forms), 9.75-9.90 (1H, broad, Z/E forms). LCMS t_{R} (min) 2.14. MS (APCI), m/z 437.59 [M+H]⁺.

### 37-Ethoxy-N,N′-bis-(1′,3′-dihydro-benzoimidazol-2′-one-5′-yl)-[1,3,5]triazine-2,4-diamine

A mixture of compound I-22 (194 mg, 1.0 mmol), 5-amino-1,3-dihydro-benzoimidazol-2-one (300 mg, 2.0 mmol), K₂CO₃ (834 mg, 6.0 mmol) and DMSO (0.7 mL) was stirred 100° C. for 6 hours, cooled to room temperature and diluted with water. The formed solid was collected by filtration and washed with mixture of acetone/ethanol (1/1) giving the compound. Yield 276 mg, 66%. ¹H-NMR (400 MHz, DMSO-D₆) δ_{H}: 1.30 (3H, t, J=7.5 Hz), 3.45 (4H, broad), 4.31 (2H, q, J=7.5 Hz), 6.67-6.82 (2H, d, J=8.5 Hz, Z/E forms), 7.03-7.22 (2H, d, J=8.5 Hz, Z/E forms), 7.33-7.73 (2H, broad, Z/E forms), 9.25 (2H, broad, Z/E forms). LCMS t_{R} (min): 1.32. MS (APCI), m/z 420.01 [M+H]⁺. HPLC t_{R} (min): 7.20. Mₚ 244-246° C.

### 38. (4,6-Bis-benzyloxy-[1,3,5]triazin-2-yl)-(4-fluoro-benzyl)-amine

To a suspension of sodium hydride (110 mg, 4.58 mmol) in THF (5 mL) benzyl alcohol (290 mg, 2.68 mmol) was added. The mixture was stirred at room temperature for 15 minutes. Then (4-(4-fluorobenzyloxy)-6-chloro-[1,3,5]triazin-2-yl)-(4-fluoro-benzyl)-amine (300 mg, 0.89 mmol) was added to the obtained solution. The resulting mixture was stirred at refluxing for 5 min, diluted with water. The formed solid was collected by filtration, washed with water. Recrystallization form ethanol gave the compound. Yield 205 mg, 55%. ¹H-NMR (400 MHz, DMSO-D₆) δ_{H}: 4.48 (2H, broad), 5.32 (4H, s), 7.10 (2H, m, J=8.5 Hz), 7.33 (10H, broad), 7.40 (2H, broad m), 8.40 (1H, broad). MW 416.46. LCMS t_{R} (min): 2.11. MS (APCI), m/z 417.01 [M+H]⁺. HPLC t_{R} (min): 16.78. M_{P} 157-159° C.

### 39. (4,6-Bis-benzoimidazol-1-yl-[1,3,5]triazin-2-yl)-(4-fluoro-benzyl)-amine

A mixture of (4-(4-fluorobenzyloxy)-6-chloro-[1,3,5]triazin-2-yl)-(4-fluoro-benzyl)-amine (300 mg, 0.89 mmol), benzoimidazole (126 mg, 1.07 mmol), K₂CO₃ (250 mg, 1.81 mmol) and DMSO (2 mL) was stirred at 90° C. for 30 minutes and cooled to room temperature. The formed solid was collected by filtration, washed with water and recrystallized from a mixture ethanol/DMF (1/2) giving the compound. Yield 127 mg, 33%. ¹H-NMR (400 MHz, DMSO-D₆) δ_{H}: 4.80 (2H, d, J=7.5 Hz), 7.20 (2H, dd, J=8.5/8.0 Hz), 7.43 (4H, m), 7.55 (2H, m), 7.80 (2H, t, J=8.5 Hz), 8.39 (1H, d, J=8.5 Hz), 8.73 (1H, d, J=8.5 Hz), 9.37 (2H, m), 9.42 (1H, broad). MW 436.46. LCMS t_{R} (min): 1.98. MS (APCI), m/z 437.25 [M+H]⁺. HPLC t_{R} (min): 15.32. M_{P} 296-298° C.

### 40. 6-(2,2,2-Trifluoro-ethoxy)-N-(3-trifluoromethyl-phenyl)-[1,3,5]triazine-2,4-diamine

¹H-NMR (400 MHz, DMSO-D₆) δ_{H}: 4.91 (2H, broad q, J=7.5 Hz), 7.21 (2H, broad peak, Z/E forms), 7.32 (1H, broad d, J=8.5 Hz), 7.48 (1H, t, J=8.5 Hz), 7.95 (1H, d, J=8.5 Hz), 8.09 (1H, s), 9.79 (1H, broad peak, Z/E forms). MW 353.23. LCMS t_{R} (min): 1.82. MS (APCI+), m/z 354.16 [M+H]⁺. HPLC t_{R} (min): 15.03. Mp 133-135° C.

### 41. 6-Ethoxy-N-(1-methyl-2-morpholin-4-ylmethyl-1H-benzoimidazol-5-yl)-N′-thiophen-2-ylmethyl-[1,3,5]triazine-2,4-diamine

¹H-NMR (400 MHz, CDCl₃) δ_{H}: 1.40 (3H, t, J=7.5 Hz), 2.54 (4H, m), 3.71 (4H, m), 3.82 (2H, s), 3.88 (3H, s), 4.41 (2H, broad), 4.83 (2H, d, J=7.5 Hz), 5.43 (1H, broad), 6.95 (1H, t, J=5.4/4.0 Hz), 6.98 (1H, broad), 7.01 (1H, d, J=4.0 Hz), 7.21 (1H, d, J=3.5 Hz), 7.25 (1H, m), 7.41 (1H, broad), 8.01 (1H, broad). LCMS t_{R} (min): 1.49. MS (APCI), m/z 481.09 [M+H]⁺. HPLC t_{R} (min): 9.55. Mₚ 177-179° C.

### 42. 4-(2,3-Dihydro-benzo[1,4]dioxin-6-ylamino)-6-[(thiophen-2-ylmethyl)-amino]-[1,3,5]triazine-2-carbonitrile

¹H-NMR (400 MHz, DMSO-D₆) δ_{H}: 4.21 (4H, m), 4.68 (2H, d, J=7.5 Hz), 6.78 (1H, d, J=8.5 Hz), 6.97 (1H, broad), 7.03 (1H, broad), 7.08 (1H, broad, Z/E forms), 7.29 (1H, broad), 7.38 (1H, broad, Z/E forms), 8.60-8.85 (1H, broad, Z/E forms), 9.80-10.09 (1H, broad, Z/E forms). LCMS t_{R} (min): 1.88. MS (APCI), m/z 367.00 [M+H]⁺. HPLC t_{R} (min): 14.15. Mₚ 184-186° C.

### 43. 6-Ethoxy-N-(3-isopropyl-phenyl)-N′-(2-pyrrolidin-1-yl-ethyl)-[1,3,5]triazine-2,4-diamine

¹H-NMR (400 MHz, DMSO-D₆) δ_{H}: 1.20 (6H, d, J=7.5 Hz), 1.28 (3H, broad t, J=7.5 Hz), 1.68 (4H, broad peak), 2.60 (2H, broad peak, Z/E forms), 2.82 (1H, m), 3.25 (2H, broad peak, Z/E forms), 3.40 (4H, broad peak, Z/E forms), 4.30 (2H, broad q, J=7.5 Hz), 6.84 (1H, d, J=8.5 Hz), 7.14 (1H, t, J=8.5 Hz), 7.49 (1H, broad peak, Z/E forms), 7.55 (1H, broad peak, Z/E forms), 7.70 (1H, broad peak, Z/E forms), 9.10-9.22 (1H, two broad peaks, Z/E forms). MW 370.50. LCMS t_{R} (min): 1.57. MS (APCI+), m/z 376.16 [M+H]⁺. HPLC t_{R} (min): 11.06. M_{P} 101-103° C.

### 44. 6-Ethoxy-N-(3-isopropyl-phenyl)-N′-(3-pyrrolidin-1-yl-propyl)-[1,3,5]triazine-2,4-diamine

¹H-NMR (400 MHz, DMSO-D₆) δ_{H}: 1.20 (6H, d, J=7.5 Hz), 1.28 (3H, broad t, J=7.5 Hz), 1.70 (6H, broad peak), 2.50 (6H, broad peak, Z/E forms), 2.83 (1H, m), 3.33 (2H, broad peak, Z/E forms), 4.30 (2H, broad q, J=7.5 Hz), 6.82 (1H, d, J=8.5 Hz), 7.14 (1H, t, J=8.5 Hz), 7.28-7.45 (1H, two broad peaks, Z/E forms), 7.55 (1H, broad peak, Z/E forms), 7.75 (1H, broad peak, Z/E forms), 9.05-9.22 (1H, two broad peaks, Z/E forms). MW 384.52. LCMS t_{R} (min): 1.62. MS (APCI+), m/z 385.17 [M+H]⁺. HPLC t_{R} (min): 10.62. M_{P} 115-116° C.

### 45. N-Ethyl-N′-(4-methoxy-phenyl)-N″-(2-pyrrolidin-1-yl-ethyl)-[1,3,5]triazine-2,4,6-triamine

¹H-NMR (400 MHz, DMSO-D₆) δ_{H}: 1.18 (3H, t, J=7.5 Hz), 1.68 (4H, m), 2.51 (2H, t, J=7.5 Hz), 3.22 (6H, m), 3.35 (2H, broad), 3.71 (3H, s), 6.20-6.70 (2H, broad, Z/E forms), 6.76 (2H, d, J=8.5 Hz), 7.61 (2H, d, J=8.5 Hz, broad), 8.55 (1H, broad). LCMS t_{R} (min): 1.25. MS (APCI), m/z 358.15 [M+H]⁺. HPLC t_{R} (min): 7.64. Mₚ 29-31° C.

### 46. N-Furan-2-ylmethyl-N′-(4-methoxy-phenyl)-[1,3,5]triazine-2,4-diamine

A mixture of compound I-31 (470 mg, 1.9 mmol), furfuryl amine (194 mg, 2 mmol), K₂CO₃ (278 mg, 4 mmol) and DMSO (1 mL) was stirred at 90° C. for 10 hours, cooled to room temperature and diluted with water. The formed solid was collected by filtration, recrystallized from CHCl₃/MeOH and purified via preparative TLC (ethyl acetate/hexane) to give the compound (300 mg, 50%).

¹H-NMR (400 MHz, DMSO-D₆) δ_{H}: 3.74 (3H, s), 4.49 (2H, broad, Z/E forms), 6.24 (2H, broad), 6.38 (1H, broad), 6.85 (2H, d, J=8.5 Hz), 7.54 (1H, d, J=1.5 Hz), 7.59 (2H, broad, Z/E forms), 7.71-7.89 (1H, broad, Z/E forms), 8.08-8.23 (1H, broad, Z/E forms), 9.19-9.42 (1H, broad, Z/E forms). LCMS t_{R} (min): 1.54. MS (APCI), m/z 298.08 [M+H]⁺. HPLC t_{R} (min): 9.65. Mₚ 184-186° C.

### 47. N-Furan-2-ylmethyl-N′-(3-trifluoromethyl-phenyl)-[1,3,5]triazine-2,4-diamine

A solution of m-CF₃-aniline (322 mg, 2 mmol) and DIPEA (258 mg, 2 mmol) in DMSO (0.5 mL) was added dropwise to a solution of dichlorotriazine (300 mg, 2 mmol) in DMSO (0.5 mL) at 10° C. The reaction mixture was stirred at room temperature for 1 hour. Then furfuryl amine (194 mg, 2 mmol) and K₂CO₃ (278 mg, 2 mmol) were added. The obtained mixture was stirred for 1 hour at 100° C., cooled down to room temperature and diluted with water. The formed solid was collected by filtration, purified by column chromatography (silica gel, ethyl acetate), preparative TLC (ethyl acetate) and recrystallized from ethyl acetate to give the compound (70 mg, 10%). ¹H-NMR (400 MHz, DMSO-D₆) δ_{H}: 4.52 (2H, d, J=7.5 Hz), 6.24 (1H, broad), 6.37 (1H, broad), 7.30 (1H, d, J=8.5 Hz), 7.50 (1H, t, J=8.5 Hz), 7.53 (1H, s), 7.85-8.00 (1H, broad, Z/E forms), 8.11 (1H, broad), 8.11-8.25 (1H, broad, Z/E forms), 8.31 (1H, broad), 9.70-9.90 (1H, broad, Z/E forms). LCMS t_{R} (min): 1.79. MS (APCI), m/z 336.04 [M+H]⁺. HPLC t_{R} (min): 12.53. Mₚ 207-209° C.

### Generic Synthesis of Intermediates and Final Compounds for Table 44 (see Table 32)

TABLE 44

| En | Structure | IUPAC Name | MW | Formula |
|---|---|---|---|---|
| 1 | 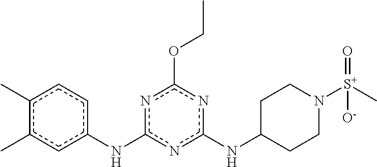 | N-(3,4-dimethylphenyl)-6- ethoxy-N′-[1- (methylsulfonyl)piperidin-4-yl]- 1,3,5-triazine-2,4-diamine | 420.5 | C19H28N6O3S |
| 2 | 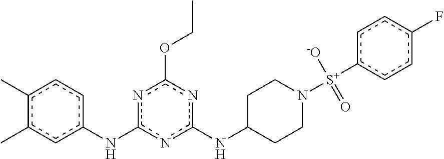 | N-(3,4-dimethylphenyl)-6- ethoxy-N′-{1-[(4- fluorophenyl)sulfonyl]piperidin- 4-yl}-1,3,5-triazine-2,4-diamine | 500.6 | C24H29FN6O3S |
| 3 | 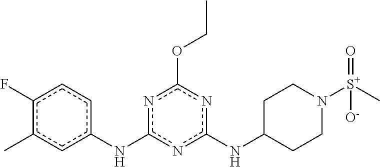 | 6-ethoxy-N-(4-fluoro-3- methylphenyl)-N′-[1- (methylsulfonyl)piperidin-4-yl]- 1,3,5-triazine-2,4-diamine | 424.5 | C18H25FN6O3S |
| 4 | 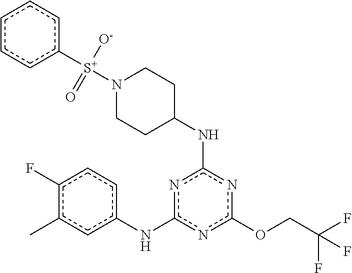 | N-(4-fluoro-3-methylphenyl)-N′- [1-(phenylsulfonyl)piperidin-4- yl]-6-(2,2,2-trifluoroethoxy)- 1,3,5-triazine-2,4-diamine | 540.5 | C23H24F4N6O3S |
| 5 | 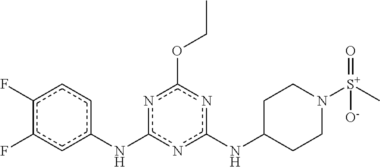 | N-(3,4-difluorophenyl)-6- ethoxy-N′-[1- (methylsulfonyl)piperidin-4-yl]- 1,3,5-triazine-2,4-diamine | 428.5 | C17H22F2N6O3S |
| 6 | 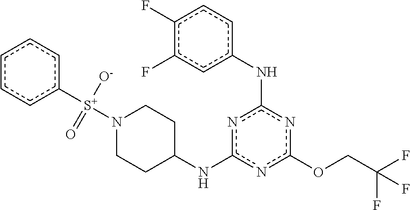 | N-(3,4-difluorophenyl)-N′-[1- (phenylsulfonyl)piperidin-4-yl]- 6-(2,2,2-trifluoroethoxy)-1,3,5- triazine-2,4-diamine | 544.5 | C22H21F5N6O3S |
| 7 | 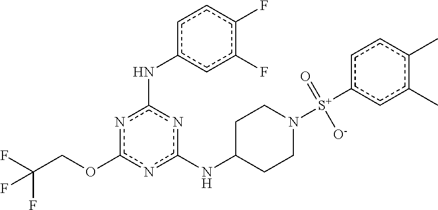 | N-(3,4-difluorophenyl)-N′-{1- [(3,4- dimethylphenyl)sulfonyl]piperidin- 4-yl}-6-(2,2,2-trifluoroethoxy)- 1,3,5-triazine-2,4-diamine | 572.6 | C24H25F5N6O3S |
| 8 | 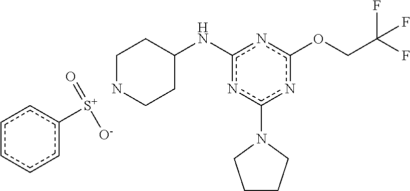 | N-[1-(phenylsulfonyl)piperidin- 4-yl]-4-(pyrrolidin-1-yl)-6-(2,2,2- trifluoroethoxy)-1,3,5-triazin-2- amine | 486.5 | C20H25F3N6O3S |
| 9 | 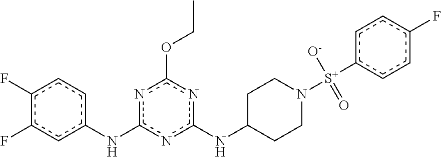 | N-(3,4-difluorophenyl)-6- ethoxy-N′-{1-[(4- fluorophenyl)sulfonyl]piperidin- 4-yl}-1,3,5-triazine-2,4-diamine | 508.5 | C22H23F3N6O3S |
| 10 | 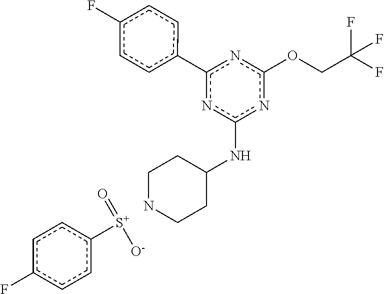 | 4-(4-fluorophenyl)-N-{1-[(4- fluorophenyl)sulfonyl]piperidin- 4-yl}-6-(2,2,2-trifluoroethoxy)- 1,3,5-triazin-2-amine | 529.5 | C22H20F5N5O3S |
| 11 | 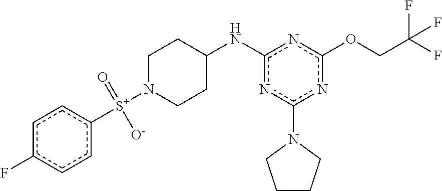 | N-{1-[(4- fluorophenyl)sulfonyl]piperidin- 4-yl}-4-(pyrrolidin-1-yl)-6-(2,2,2- trifluoroethoxy)-1,3,5-triazin-2- amine | 504.5 | C20H24F4N6O3S |
| 12 | 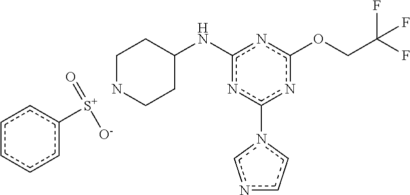 | 4-(1H-imidazol-1-yl)-N-[1- (phenylsulfonyl)piperidin-4-yl]- 6-(2,2,2-trifluoroethoxy)-1,3,5- triazin-2-amine | 483.5 | C19H20F3N7O3S |
| 13 | 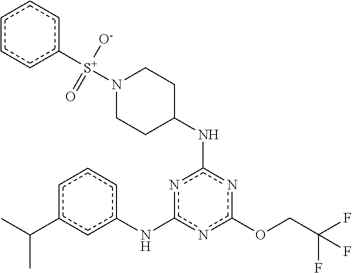 | N-[1-(phenylsulfonyl)piperidin- 4-yl]-N′-[3-(propan-2-yl)phenyl]- 6-(2,2,2-trifluoroethoxy)-1,3,5- triazine-2,4-diamine | 550.6 | C25H29F3N6O3S |
| 14 | 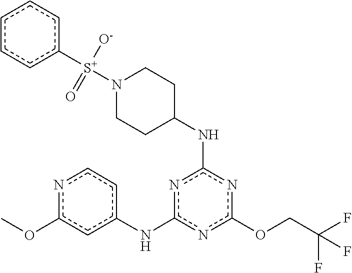 | N-(2-methoxypyridin-4-yl)-N′-[1- (phenylsulfonyl)piperidin-4-yl]- 6-(2,2,2-trifluoroethoxy)-1,3,5- triazine-2,4-diamine | 539.5 | C22H24F3N7O4S |
| 15 | 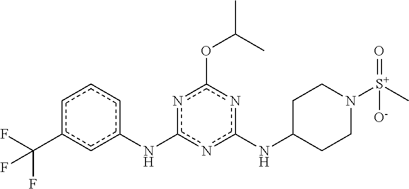 | N-[1-(methylsulfonyl)piperidin- 4-yl]-6-(propan-2-yloxy)-N′-[3- (trifluoromethyl)phenyl]-1,3,5- triazine-2,4-diamine | 474.5 | C19H25F3N6O3S |
| 16 | 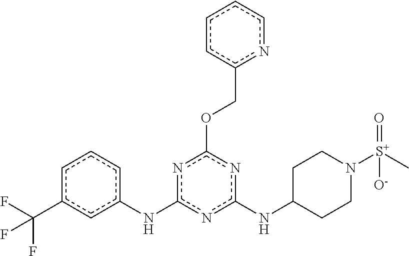 | N-[1-(methylsulfonyl)piperidin- 4-yl]-6-(pyridin-2-ylmethoxy)-N′- [3-(trifluoromethyl)phenyl]- 1,3,5-triazine-2,4-diamine | 523.5 | C22H24F3N7O3S |
| 17 | 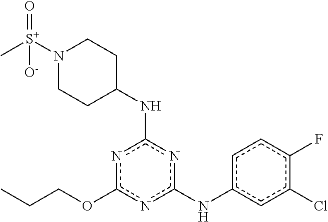 | N-(3-chloro-4-fluorophenyl)-N′- [1-(methylsulfonyl)piperidin-4- yl]-6-propoxy-1,3,5-triazine-2,4- diamine | 458.9 | C18H24ClFN6O3S |
| 18 | 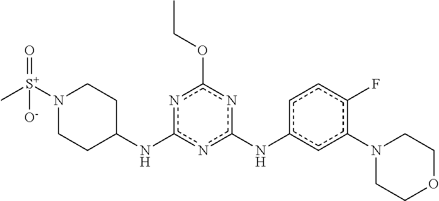 | 6-ethoxy-N-[4-fluoro-3- (morpholin-4-yl)phenyl]-N′-[1- (methylsulfonyl)piperidin-4-yl]- 1,3,5-triazine-2,4-diamine | 495.6 | C21H30FN7O4S |
| 19 | 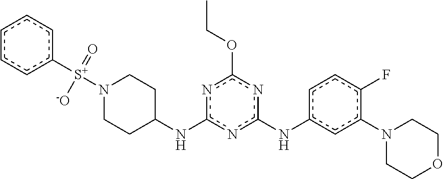 | 6-ethoxy-N-[4-fluoro-3- (morpholin-4-yl)phenyl]-N′-[1- (phenylsulfonyl)piperidin-4-yl]- 1,3,5-triazine-2,4-diamine | 557.6 | C26H32FN7O4S |

### Procedures and Analytical Data for Table 44.

### 1. N-(3,4-Dimethyl-phenyl)-6-ethoxy-N′-(1-methanesulfonyl-piperidin-4-yl)-[1,3,5]triazine-2,4-diamine

¹H-NMR (400 MHz, DMSO-D₆) δ_{H}: 1.27 (3H, broad t, J=7.5 Hz), 1.57 (2H, broad), 1.95 (2H, broad), 2.16 (3H, s), 2.19 (3H, s), 2.85 (5H, m), 3.59 (2H, m), 3.90 (1H, broad), 4.30 (2H, broad q, J=7.5 Hz), 7.00 (1H, broad d, J=8.5 Hz, Z/E forms), 7.22-7.33 (1H, broad, Z/E forms), 7.39-7.44 (1H, broad, Z/E forms), 7.49-7.55 (1H, broad, Z/E forms), 8.99-9.18 (1H, broad, Z/E forms). MW 420.54. LCMS t_{R} (min): 1.81. MS (APCI), m/z 421.15 [M+H]⁺. HPLC t_{R} (min): 12.15. M_{P} 177-179° C.

### 2. N-(3,4-Dimethyl-phenyl)-6-ethoxy-N′-[1-(4-fluoro-benzenesulfonyl)-piperidin-4-yl]-[1,3,5]triazine-2,4-diamine

¹H-NMR (400 MHz, DMSO-D₆) δ_{H}: 1.25 (3H, t, J=7.5 Hz), 1.58 (2H, m), 1.92 (2H, broad peak, Z/E forms), 2.02-2.17 (3H, two broad peaks, Z/E forms), 2.35-2.45 (3H, two broad peaks, Z/E forms), 2.49 (2H, m), 3.60 (2H, m), 3.72 (1H, broad peak, Z/E forms), 4.25 (2H, broad q, J=7.5 Hz), 6.90-7.03 (1H, two broad peaks, Z/E forms), 7.22 (1H, broad peak, Z/E forms), 7.40 (2H, broad peak, Z/E forms), 7.50 (2H, broad peak, Z/E forms), 7.85 (2H, broad peak, Z/E forms), 8.98-9.13 (1H, two broad peak, Z/E forms). MW 500.59. LCMS t_{R} (min): 2.00. MS (APCI+), m/z 501.15 [M+H]⁺. HPLC t_{R} (min): 14.76. M_{P} 231-233° C.

### 3. 6-Ethoxy-N-(4-fluoro-3-methyl-phenyl)-N′-(1-methanesulfonyl-piperidin-4-yl)-[1,3,5]triazine-2,4-diamine

Yield 147 mg, 53%. ¹H-NMR (400 MHz, DMSO-D₆) δ_{H}: 1.28 (3H, broad peak, Z/E forms), 1.59 (2H, broad peak, Z/E forms), 1.95 (2H, broad peak, Z/E forms), 2.21 (3H, s), 2.82 (2H, broad peak, Z/E forms), 2.88 (3H, s), 3.59 (2H, m), 3.90 (1H, broad peak, Z/E forms), 4.30 (2H, broad q, J=7.5 Hz), 7.02 (1H, broad peak, Z/E forms), 7.30-7.41 (1H, two broad peaks, Z/E forms), 7.50-7.59 (1H, two broad peaks, Z/E forms), 7.64 (1H, broad peak, Z/E forms), 9.13-9.32 (1H, two broad peaks, Z/E forms). MW 424.50. LCMS t_{R} (min): 1.76. MS (APCI+), m/z 425.13 [M+H]⁺. HPLC t_{R} (min): 12.13. M_{P} 188-190° C.

### 4. N-(1-Benzenesulfonyl-piperidin-4-yl)-N′-(4-fluoro-3-methyl-phenyl)-6-(2,2,2-trifluoro-ethoxy)-[1,3,5]triazine-2,4-diamine

¹H-NMR (400 MHz, DMSO-D₆) δ_{H}: 1.58 (2H, broad peak, Z/E forms), 1.93 (2H, broad peak, Z/E forms), 2.05-2.20 (4H, two broad peaks, Z/E forms), 3.62 (2H, m), 3.75 (2H, broad peak, Z/E forms), 4.90 (2H, broad q, J=7.5 Hz), 6.98 (1H, broad peak, Z/E forms), 7.30-7.52 (1H, two broad peaks, Z/E forms), 7.52 (1H, broad peak, Z/E forms), 7.66 (3H, broad peak, Z/E forms), 7.76 (3H, broad peak, Z/E forms), 9.30-9.50 (1H, two broad peaks, Z/E forms). MW 540.54. LCMS t_{R} (min): 2.03. MS (APCI+), m/z 541.13 [M+H]⁺. HPLC t_{R} (min): 16.95. M_{P} 204-206° C.

### 5. N-(1-Benzenesulfonyl-piperidin-4-yl)-N′-(3,4-difluoro-phenyl)-6-(2,2,2-trifluoro-ethoxy)-[1,3,5]triazine-2,4-diamine

¹H-NMR (400 MHz, DMSO-D₆) δ_{H}: 1.59 (2H, broad peak, Z/E forms), 1.93 (2H, broad peak, Z/E forms), 2.48 (2H, broad peak), 3.62 (2H, m), 3.78 (1H, broad peak, Z/E forms), 4.92 (2H, broad q, J=7.5 Hz, Z/E forms), 7.28 (1H, m), 7.30-7.38 (1H, two broad peaks, Z/E forms), 7.66-7.72 (2H, two broad peaks, Z/E forms), 7.76 (4H, broad peaks, Z/E forms), 7.83-7.98 (1H, two broad peak, Z/E forms), 9.60-9.74 (1H, two broad peaks, Z/E forms). MW 544.20. LCMS t_{R} (min): 2.03. MS (APCI+), m/z 545.21 [M+H]⁺. HPLC t_{R} (min): 16.18. M_{P} 224-225° C.

### 6. N-(3,4-Difluoro-phenyl)-N′-[1-(3,4-dimethyl-benzenesulfonyl)-piperidin-4-yl]-6-(2,2,2-trifluoro-ethoxy)-[1,3,5]triazine-2,4-diamine

¹H-NMR (400 MHz, DMSO-D₆) δ_{H}: 1.58 (2H, m), 1.92 (2H, broad peak, Z/E forms), 2.30 (6H, s), 2.41 (2H, m), 3.60 (2H, m), 3.72 (1H, broad peak, Z/E forms), 4.90 (2H, broad q), 7.22-7.32 (2H, two broad peaks, Z/E forms), 7.39 (1H, broad peak, Z/E forms), 7.46 (1H, m), 7.51 (1H, m), 7.70-7.85 (1H, two broad peaks, Z/E forms), 7.85-7.98 (1H, two broad peaks, Z/E forms), 9.60-9.75 (1H, two broad peaks, Z/E forms). MW 572.56. LCMS t_{R} (min): 2.13. MS (APCI+), m/z 573.10 [M+H]⁺. HPLC t_{R} (min): 17.91. M_{P} 228-230° C.

### 7. (1-Benzenesulfonyl-piperidin-4-yl)-[4-pyrrolidin-1-yl-6-(2,2,2-trifluoro-ethoxy)-[1,3,5]triazin-2-yl]-amine

A solution of pyrrolidine (0.09 mL, 77 mg, 1.08 mmol) and DIPEA (188 mg, 1.08 mmol) in dry THF (10 mL) was added gradually to a solution of cyanuric chloride (199 mg, 1.08 mmol) in THF (15 mL) at −20° C. within 1.5 hour. Then, the reaction mixture was stirred at this temperature for 2.5 hours. Then, it was allowed to warm up to 0° C., and the mixture of 1-benzenesulfonyl-piperidin-4-ylamine hydrochloride (300 mg, 1.08 mmol) and DIPEA (376 mg, 2.16 mmol) in THF (10 mL) was added to the reaction mixture within 1 hour. The reaction mixture was stirred at 0° C. for 1 hour, allowed to warm to room temperature and left overnight. Then, the mixture was diluted with water and extracted with ethyl acetate. The organic phase was washed with water, brine, dried over sodium sulfate, and concentrated. Purification by column chromatography on silica gel (ethyl acetate/hexane) gave compound (1-Benzenesulfonyl-piperidin-4-yl)-(4-chloro-6-pyrrolidin-1-yl-[1,3,5]triazin-2-yl)-amine. Yield 169 mg, 37%.

A mixture of (1-Benzenesulfonyl-piperidin-4-yl)-(4-chloro-6-pyrrolidin-1-yl-[1,3,5]triazin-2-yl)-amine (169 mg, 0.40 mmol), 2,2,2-trifluoroethanol (0.102 mL, 140 mg, 1.40 mmol), K₂CO₃ (194 mg, 1.40 mmol) in DMSO (2.5 mL) was stirred at 80° C. for 4.5 hours and left at room temperature overnight. Then, additional amount of 2,2,2-trifluoroethanol (0.100 mL) was added, and the mixture was stirred at 80° C. for 3 hours, cooled down to room temperature and diluted with water. The formed precipitate was filtered off, washed with water and dried on air and purified by column chromatography on silica gel (ethyl acetate) that gave the compound. Yield 138 mg, 71%. ¹H-NMR (400 MHz, DMSO-D₆) δ_{H}: 1.52 (2H, m), 1.88 (6H, m), 2.50 (2H, m), 3.40 (4H, broad peak), 3.57 (2H, broad peak), 3.72 (1H, broad peak, Z/E forms), 4.85 (2H, broad q, J=7.5 Hz), 7.22-7.33 (1H, two broad peaks, Z/E forms), 7.65 (2H, superposition of two t, J=8.5 Hz, Z/E forms), 7.73 (3H, m). MW 486.51. LCMS t_{R} (min): 2.01. MS (APCI+), m/z 487.16 [M+H]⁺. HPLC t_{R} (min): 15.33. M_{P} 184-186° C.

### 8. N-(3,4-Difluoro-phenyl)-6-ethoxy-N′-[1-(4-fluoro-benzenesulfonyl)-piperidin-4-yl]-[1,3,5]triazine-2,4-diamine

To a suspension of 4-fluoro-benzenesulfonyl-piperidin-4-ylamine hydrochloride (155 mg, 0.52 mmol) in MeCN (5 mL) NEt₃ (106 mg, 1.05 mmol) was added. The resulting mixture was stirred for 5 minutes at room temperature and N-(3,4-Difluoro-phenyl)-6-ethoxy-4-chloro-[1,3,5]triazine-2,4-diamine (150 mg, 0.52 mmol) was added. The reaction mixture was refluxed for 2 hours (TLC control). Then, the reaction mixture was concentrated, washed with small amount of water and with dichloromethane and dried that gave the compound. Yield 120 mg, 45%. ¹H-NMR (400 MHz, DMSO-D₆) δ_{H}: 1.25 (3H, broad t, J=7.5 Hz), 1.55 (2H, m), 1.90 (2H, m), 2.49 (2H, m), 3.60 (2H, m), 3.74 (1H, broad peak), 4.28 (2H, d, J=7.5 Hz), 7.29 (2H, broad peak, Z/E forms), 7.40 (1H, broad peak), 7.50 (2H, broad peak, Z/E forms), 7.82 (2H, broad peaks, Z/E forms), 7.90-8.01 (1H, two broad peaks, Z/E forms), 9.40-9.53 (1H, two broad peaks, Z/E forms). MW 508.52. LCMS t_{R} (min): 2.01. MS (APCI+), m/z 509.10 [M+H]⁺. HPLC t_{R} (min): 15.50. M_{P} 240-242° C.

### 9. N-(3,4-Difluoro-phenyl)-6-ethoxy-N′-(1-methanesulfonyl-piperidin-4-yl)-[1,3,5]triazine-2,4-diamine

¹H-NMR (400 MHz, DMSO-D₆) δ_{H}: 1.29 (3H, broad peak), 1.57 (2H, broad peak), 1.95 (2H, broad peak), 2.85 (5H, broad peak), 3.60 (2H, broad peak), 3.90 (1H, broad peak), 4.30 (2H, broad peak), 7.31 (1H, m), 7.43 (1H, broad peak, Z/E forms), 7.54 (1H, broad peak, Z/E forms), 7.91-8.05 (1H, two broad peaks, Z/E forms), 9.48-9.62 (1H, two broad peaks, Z/E forms). MW 428.46. LCMS t_{R} (min): 1.78. MS (APCI+), m/z 429.11 [M+H]⁺. HPLC t_{R} (min): 12.73. M_{P} 197-199° C.

### 10. [1-(4-Fluoro-benzenesulfonyl)-piperidin-4-yl]-[4-(4-fluoro-phenyl)-6-(2,2,2-trifluoro-ethoxy)-[1,3,5]triazin-2-yl]-amine

A mixture of [1-(4-Fluoro-benzenesulfonyl)-piperidin-4-yl]-[4-(chloro)-6-(2,2,2-trifluoro-ethoxy)-[1,3,5]triazin-2-yl]-amine (270 mg, 0.57 mmol), p-fluoro-phenyl-boronic acid (88 mg, 0.63 mmol), Pd(PPh₃)₄ (70 mg) and Na₂CO₃ (74 mg, 0.70 mmol) in dioxane and water (10/1; 7 mL) was refluxed under argon for 4 hours, cooled down to room temperature, filtered and the filtrate was concentrated. The dark residue was purified by column chromatography on silica gel (dichloromethane), and crystallized twice with ether and with a mixture of MeCN/H₂O (2/1) that gave the compound (114 mg, 38%). ¹H-NMR (400 MHz, DMSO-D₆) δ_{H}: 1.61 (2H, broad), 1.98 (2H, broad peak), 2.62 (2H, m), 3.60 (2H, m), 3.90-4.08 (1H, broad peak, Z/E forms), 5.07 (2H, q, J=8.5 Hz), 7.32 (2H, superposition of two m, Z/E forms), 7.49 (2H, superposition of two m, Z/E forms), 7.85 (2H, broad peak, Z/E forms), 8.14-8.25 (1H, two d, J=7.5 Hz, Z/E forms), 8.38 (2H, broad peak, Z/E forms). MW 529.49. LCMS t_{R} (min): 2.12. MS (APCI+), m/z 530.10 [M+H]⁺. HPLC t_{R} (min): 17.73. M_{P} 183-185° C.

### 11. (1-Benzenesulfonyl-piperidin-4-yl)-[4-pyrrolidin-1-yl-6-(2,2,2-trifluoro-ethoxy)-[1,3,5]triazin-2-yl]-amine

To a solution of [1-(4-Fluoro-benzenesulfonyl)-piperidin-4-yl]-[4-(chloro)-6-(2,2,2-trifluoro-ethoxy)-[1,3,5]triazin-2-yl]-amine (350 mg, 0.74 mmol) in MeCN (5 mL) a solution of pyrrolidine (0.07 mL, 0.82 mmol) and DIPEA (0.14 mL, 0.82 mmol) in MeCN (5 mL) was added. The resulting mixture was stirred at room temperature for 1.5 hour (TLC control). Then, the reaction mixture was diluted with water (40 mL), and in 30 minutes the formed precipitate was filtered off, washed with water and hexane, and dried in vacuum. Purification by column chromatography on silica gel (CH₂Cl₂/ethyl acetate) gave the compound as white crystalline powder. Yield 280 mg, 75%. ¹H-NMR (400 MHz, DMSO-D₆) δ_{H}: 1.52 (2H, broad peak, Z/E forms), 1.86 (6H, broad peak, Z/E forms), 3.18 (2H, broad peak, Z/E forms) 3.40 (4H, broad peak, Z/E forms), 3.40-3.55 (2H, two broad peaks, Z/E forms), 3.55-3.78 (1H, two broad peaks, Z/E forms), 4.87 (2H, q, J=7.5 Hz), 7.30 (1H, broad d, J=7.5 Hz, Z/E forms), 7.48 (2H, d/d, J=8.5/8.0 Hz, Z/E forms), 7.82 (2H, m). MW 504.51. LCMS t_{R} (min): 2.01. MS (APCI+), m/z 505.15 [M+H]⁺. HPLC t_{R} (min): 15.85. M_{P} 212-213° C.

### 12. (1-Benzenesulfonyl-piperidin-4-yl)-[4-imidazol-1-yl-6-(2,2,2-trifluoro-ethoxy)-[1,3,5]triazin-2-yl]-amine

To a solution of (1-Benzenesulfonyl-piperidin-4-yl)-(2,4-dichloro-[1,3,5]triazin-2-yl)-amine (311 mg, 0.8 mmol) and DIPEA (110 mg, 0.85 mmol) in THF (5 mL) a solution of imidazole (55 mg, 0.8 mmol) in THF (5 mL) was added dropwise at 0° C. The reaction mixture was stirred at room temperature for 16 hours, diluted with water and extracted with dichloromethane. The organic phase was dried over sodium sulfate and concentrated. Purification by column chromatography (silica gel, ethyl acetate/hexane, 1/1) gave mono-chloro-compound (200 mg, 59%).

A mixture of the mono-chloro-compound (200 mg, 0.48 mmol), 2,2,2-trifluoroethanol (143 mg, 1.43 mmol), K₂CO₃ (132 mg, 0.95 mmol) in DMSO (2 mL) and MeCN (4 mL) was stirred at 100° C. for 1 hour. Then, the mixture was cooled down to room temperature and diluted with water. The formed precipitate was filtered off, and purified by column chromatography on silica gel (hexane/ethyl acetate) that gave a final compound. Yield 80 mg, 35%. ¹H-NMR (400 MHz, DMSO-D₆) δ_{H}: 1.60 (2H, m), 2.58 (2H, m), 3.62 (2H, m), 3.82-4.00 (1H, broad, Z/E forms), 5.09 (2H, superposition of two q, J=7.5 Hz), 7.08-7.11 (1H, two peaks, Z/E forms), 7.67 (2H, broad peak, Z/E forms), 7.77 (5H, broad peak, Z/E forms), 7.80-7.88 (1H, two peaks, Z/E forms), 8.45-8.55 (1H, two d, Z/E forms), 8.48-8.60 (1H, two s, Z/E forms). MW 483.47. LCMS t_{R} (min): 1.69. MS (APCI+), m/z 484.17 [M+H]⁺. HPLC t_{R} (min): 11.45. M_{P} 201-203° C.

### 13. N-(1-Benzenesulfonyl-piperidin-4-yl)-N′-(3-isopropyl-phenyl)-6-(2,2,2-trifluoro-ethoxy)-[1,3,5]-triazine-2,4-diamine

¹H-NMR (400 MHz, DMSO-D₆) δ_{H}: 1.05-1.20 (6H, two broad peaks, Z/E forms), 1.60 (2H, m), 1.92 (2H, m), 2.40 (2H, m), 2.70-2.84 (1H, two broad peaks, Z/E forms), 3.62 (2H, m), 3.80 (1H, broad), 4.91 (2H, broad q, J=7.5 Hz), 6.81-6.90 (1H, broad, Z/E forms), 7.08-7.19 (1H, broad), 7.26-7.42 (1H, broad, Z/E forms), 7.48-7.67 (1H, broad, Z/E forms), 7.68 (3H, broad), 7.75 (3H, broad), 9.27-9.48 (1H, two broad peaks). MW 550.61. LCMS t_{R} (min): 2.16. MS (APCI), m/z 551.36 [M+H]⁺. HPLC t_{R} (min): 17.42. M_{P} 193-195° C.

### 14. N*2*-(1-Benzenesulfonyl-piperidin-4-yl)-N*4*-(2-methoxy-pyridin-4-yl)-6-(2,2,2-trifluoro-ethoxy)-1,3,5-triazine-2,4-diamine

¹H-NMR (400 MHz, DMSO-D₆) δ_{H}: 1.60 (2H, m), 1.95 (2H, m), 2.52 (2H, m), 3.64 (2H, m), 3.74-3.82 (5H, superposition of two s, Z/E forms), 4.93 (2H, broad q, J=7.5 Hz), 7.10-7.28 (1H, two broad peaks, Z/E forms), 7.32 (1H, broad), 7.68 (2H, broad peaks), 7.77 (3H, broad peaks), 7.91 (2H, broad peaks), 9.78-9.90 (1H, two broad peaks, Z/E forms). MW 539.5. LCMS t_{R} (min): 1.78. MS (APCI+), m/z 540.14 [M+H]⁺. HPLC t_{R} (min): 12.13. M_{P} 213-215° C.

### 15. 6-Isopropoxy-N-(1-methanesulfonyl-piperidin-4-yl)-N′-(3-trifluoromethyl-phenyl)-[1,3,5]triazine-2,4-diamine

¹H-NMR (400 MHz, DMSO-D₆) δ_{H}: 1.28 (6H, broad peak, Z/E forms), 1.61 (2H, m), 1.92 (2H, m), 2.71 (2H, m), 2.73 (3H, s), 3.58 (2H, m), 3.91 (1H, broad peak, Z/E forms), 5.19 (1H, broad m), 7.21 (1H, broad peak, Z/E forms), 7.41 (2H, broad peak, Z/E forms), 7.41-7.81 (1H, two broad peaks, Z/E forms), 8.11-8.31 (1H, two broad peaks, Z/E forms), 9.41-9.68 (1H, two broad peaks, Z/E forms). MW 474.50. LCMS t_{R} (min): 1.93. MS (APCI+), m/z 474.95 [M+H]⁺. HPLC t_{R} (min): 14.85. M_{P} 220-222° C.

### 16. N-(1-Methanesulfonyl-piperidin-4-yl)-6-(pyridin-2-ylmethoxy)-N′-(3-trifluoromethyl-phenyl)-[1,3,5]triazine-2,4-diamine

Sodium metal (46 mg, 0.5 mmol) was dissolved in a mixture of THF (1 mL) and pyridin-2-yl-methanol (2 mL). N-(1-Methanesulfonyl-piperidin-4-yl)-6-(chloro)-N′-(3-trifluoromethyl-phenyl)-[1,3,5]triazine-2,4-diamine (225 mg, 0.5 mmol) was added to the mixture and the resulting mixture was stirred for 3 hours at 70° C., diluted with water (20 mL). The obtained precipitate was filtered, washed with water and dried on air. Purification by column chromatography on silica gel (acetone/chloroform) gave the compound. Yield 180 mg, 69%. ¹H-NMR (400 MHz, DMSO-D₆) δ_{H}: 1.60 (2H, m), 1.82-1.98 (2H, two broad peaks, Z/E forms), 2.80 (2H, m), 2.90 (3H, s), 3.60 (2H, broad), 3.84-3.98 (1H, broad, Z/E forms), 5.45 (2H, s), 7.30 (1H, broad peak, Z/E forms), 7.34 (1H, d, J=8.5 Hz), 7.44 (1H, t, J=8.5 Hz), 7.50 (1H, broad peak, Z/E forms), 7.46-7.67 (1H, two broad peaks, Z/E forms), 7.83 (1H, broad t, J=8.0 Hz), 7.85-8.02 (1H, two broad peaks, Z/E forms), 8.13-8.29 (1H, two broad peaks, Z/E forms), 8.57 (1H, d, J=5.0 Hz), 9.60-9.80 (1H, two broad peaks, Z/E forms). MW 523.54. LCMS t_{R} (min): 1.72. MS (APCI+), m/z 524.11 [M+H]⁺. HPLC t_{R} (min): 11.22. M_{P} 160-162° C.

### 17. N-(3-Chloro-4-fluoro-phenyl)-N′-(1-methanesulfonyl-piperidin-4-yl)-6-propoxy-[1,3,5]triazine-2,4-diamine

NaH (73 mg, 60% in oil, 3.04 mmol) was added to a solution of propane-1-ol (110 mg, 1.83 mmol) in THF (5 mL) at 0° C. The mixture was stirred for 15 minutes at 0° C. Then a solution of N-(3-Chloro-4-fluoro-phenyl)-N′-(1-methanesulfonyl-piperidin-4-yl)-6-chloro-[1,3,5]triazine-2-amine (265 mg, 0.65 mmol) in THF (10 mL) was added to the obtained suspension at 0° C. The final reaction mixture was stirred at refluxing for 3 hours, cooled to room temperature, diluted with water, extracted with dichloromethane. The combined organic phases were concentrated at reduced pressure and purified by column chromatography on silica gel (45% ethyl acetate/hexane) and recrystallization (THF/water) to give final compound as white crystals. Yield 137 mg, 49%. ¹H-NMR (400 MHz, DMSO-D₆) δ_{H}: 0.94 (3H, t, J=7.5 Hz), 1.58 (2H, m), 1.69 (2H, m), 1.97 (2H, m), 2.88 (5H, superposition of s and m), 3.60 (2H, broad peak, Z/E forms), 3.90 (1H, broad peak, Z/E forms), 4.21 (2H, broad peaks), 7.30 (1H, broad peak, Z/E forms), 7.42-7.56 (1H, broad peaks, Z/E forms), 7.56-7.70 (1H, two broad peaks, Z/E forms), 8.00-8.11 (1H, two broad peaks, Z/E forms), 9.40-9.60 (1H, two broad peaks, Z/E forms). MW 458.95. LCMS t_{R} (min): 1.90. MS (APCI+), m/z 459.02, 461.01 [M+H]⁺. HPLC t_{R} (min): 14.11. M_{P} 199-201° C.

### 18. 6-Ethoxy-N-(4-fluoro-3-morpholin-4-yl-phenyl)-N′-(1-methanesulfonyl-piperidin-4-yl)-[1,3,5]triazine-2,4-diamine

To a solution of 6-chloro-N-(4-fluoro-3-morpholin-4-yl-phenyl)-N′-(1-methanesulfonyl-piperidin-4-yl)-[1,3,5]triazine-2-amine (177 mg, 0.5 mmol) in acetonitrile (3 mL) 1-methanesulfonyl-piperidin-4-ylamine hydrochloride 2 (118 mg, 0.55 mmol) and DIPEA (194 mg, 1.5 mmol) were added portionwise at room temperature. The resulting mixture was stirred at 50° C. for 2 hours and diluted with water. The residue was washed with water and hexane. Purification by column chromatography on silica gel (chloroform/acetone) and prepTLC (chloroform/ethanol, 20/1) gave the compound. Yield 39 mg, 16%. ¹H-NMR (400 MHz, DMSO-D₆) δ_{H}: 1.30 (3H, broad), 1.59 (2H, m), 1.93 (2H, m), 2.86 (5H, superposition of s and m), 3.00 (4H, m), 3.57 (2H, m), 3.74 (4H, m), 3.93 (1H, broad peak, Z/E forms), 4.30 (2H, broad q, J=7.5 Hz), 7.05 (1H, broad peak, Z/E forms), 7.23 (1H, broad peak, Z/E forms), 7.30-7.42 (1H, two broad peaks, Z/E forms), 7.49 (1H, broad peak, Z/E forms), 9.10-9.30 (1H, two broad peaks, Z/E forms). MW 495.58 LCMS t_{R} (min): 1.67. MS (APCI+), m/z 496.15 [M+H]⁺. HPLC t_{R} (min): 11.20. M_{P} 300° C. (dec).

### 19. N-(1-Benzenesulfonyl-piperidin-4-yl)-6-ethoxy-N′-(4-fluoro-3-morpholin-4-yl-phenyl)-[1,3,5]triazine-2,4-diamine

¹H-NMR (400 MHz, DMSO-D₆) δ_{H}: 1.25 (3H, t, J=7.5 Hz), 1.58 (2H, m), 1.90 (2H, m), 2.43 (2H, m), 2.89-3.02 (4H, two broad peaks, Z/E forms), 3.60 (2H, broad peak, Z/E forms), 3.66-3.77 (5H, two broad peaks, Z/E forms), 4.27 (2H, broad q, J=7.5 Hz), 6.96 (1H, broad peak, Z/E forms), 7.21 (1H, broad peak), 7.28-7.38 (1H, two broad peaks, Z/E forms), 7.66 (2H, t, J=8.5 Hz), 7.75 (3H, superposition of d and t), 9.08-9.20 (1H, two broad peaks, Z/E forms). MW 557.65. LCMS t_{R} (min): 1.89. MS (APCI+), m/z 558.17 [M+H]⁺. HPLC t_{R} (min): 13.98. M_{P} 141-143° C.

### EXAMPLES (CONT.)

### Materials and Methods for Biological Profiling of Compounds of the Invention

### Production of HCVpp

HCV pseudoparticles (HCVpp) recapitulate much of the known biology of HCV entry, including dependency on the co-receptor CD81. Functional HCVpp were produced in 293T cells by co-transfection of optimized HCV E1/E2 expression constructs and a non-replicating HIV-1-based reporter vector (pNLluc+Δ299) as described. (See, e.g., J. Dumonceaux et al., 2003; E. Cormier et al., 2004; T. Dragic et al., 1996; U.S. Patent Application No. 20050266400 to J. Dumonceaux et al., the contents of which are hereby incorporated by reference in their entirety). Plasmids for transfection included NLluc+env− vector (R. I. Conner et al., 1995) and an expression vector encoding the E1 and E2 glycoproteins from HCV (the pcDNA3.1-ΔC-E1*-E2* plasmid construct encoding HCV-ΔC-E1/E2 (HCV isolate H77, genotype 1a), lacking putative splice acceptor sites (Cormier, E. G. et al., 2004)). The HIV-1 backbone NLluc+env− contains a frameshift mutation in the HIV-1 envelope glycoprotein gene (env), which has the potential to revert. In order to generate vectors with enhanced safety properties, a 299 bp deletion in env was introduced by excision of the Nhe1/BsaB1 fragment. This construct, designated pNLluc+Δ299, encodes a packageable HIV core particle that is decorated with the E1/E2 envelope glycoproteins from HCV

The finding of a cryptic intron excision site within HCV E1 aided in the development of an HTS amenable HCVpp assay for screening small molecule compounds. This cryptic splice site is not utilized during natural infection by HCV because RNA splicing occurs in the nucleus and HCV replicates exclusively in the cytoplasm. However, in plasmid-based expression systems such as those used to generate HCVpp, RNA splicing results in the expression of aberrant, non-fusogenic forms of E1 along with native E1. When the putative splice acceptors were removed from E1/E2 by conservative mutagenesis, plasmid expression generated single E1 and E2 protein species that formed noncovalent heterodimers on the cell surface. HCVpp produced using splice-modified E1 was observed to mediate 5-10-fold higher levels of entry into cells (Dumonceaux, J., 2003; U.S. Patent Application No. 20050266400 to Dumonceaux et al., published on Dec. 1, 2005). Accordingly, the described HCVpp involved E1/E2 from the HCV genotype 1a isolate H77, modified by conservative mutagenesis to eliminate cryptic splice sites in E1, which resulted in a more uniform expression of E1 (Dumonceaux, J. et al., 2003; U.S. Patent Application No. 20050266400 to Dumonceaux et al., published on Dec. 1, 2005).

For HCVpp production, 293T cells were co-transfected with NLluc+Δ299 env⁻ reporter vector and E1/E2 expression vector (E1/E2 pcDNA 3.1) in a 1:2 or 1:3 ratio using Lipofectamine 2000 in serum-free OPTIMEM medium (Gibco BRL; Invitrogen) as described. (See, E. G. Cormier et al., 2004). Typically, 5×10⁶ 293T cells were cotransfected with 4 μg of the NLluc+Δ299(env⁻) reporter vector (R. I. Conner et al., 1995), and 8 μg of the E1/E2 expression vector in a 10 cm² dish (BD Falcon, Bedford, Mass.), (Id.), which is described in detail by Dumonceaux, J. et al., 2003, U.S. Application No. 20050266400 to Dumonceaux et al., and E. Cormier et al., 2004. Four hours post-transfection, medium (Gibco BRL; Invitrogen) supplemented with 10% FBS was added to the transfected cells. In this system, HIV-1 core particles decorated with the E1 and E2 envelope glycoproteins of HCV are shed directly into the culture medium. Cell culture supernatants containing HCVpp were collected at 48 hours post-transfection and centrifuged at 1000 rpm for 5 minutes to clarify and pellet cell debris. Clarified viral HCVpp containing supernatants were sterile filtered, aliquoted and stored at −80° C. HCVpp containing supernatants were quantified for HIV-1 p24 protein content by ELISA, for protein content by BCA assay (Pierce, Rockford, Ill.) or for E2 content by Western blot assay. In this latter assay, purified HCVpp were heat-denatured at 100° C. for 5 minutes and subjected to SDS-polyacrylamide gel electrophoresis using known quantities of purified recombinant soluble E2 (rsE2, Austral Biologicals, San Ramon, Calif.) as a standard. Proteins were transferred to nitrocellulose membranes, blocked, and then probed with anti-E2 monoclonal antibody (MAb) (MAb 303F76) followed by detection with alkaline-phosphatase-conjugated goat anti-mouse IgG.

Blocking HCV Entry into Susceptible Cells

JS-81, a human anti-CD81 monoclonal antibody and an irrelevant mouse immunoglobulin control were tested for their ability to inhibit the entry of HCV pseudoparticles (HCVpp) of different genotypes into human hepatic carcinoma cells (Hep3B). HCVpp containing supernatants were stored at −80° C. and then thawed at 25° C. for thirty minutes prior to use in the virus infection inhibition assay. Equal volumes (20 μl) of HCVpp and the cells, (2×10³ cells/well), were plated in solid white 384-well tissue culture plates (Perkin Elmer) in DMEM/2% FCS. After incubating the plates at 37° C. for 3 days, medium was removed from the wells and equal volumes of PBS and Bright-Glo (Promega, Madison Wis.) (25 μl each) were added. Viral entry was quantified by measuring luciferase activity in a simple homogeneous assay expressed as (Relative Light Units—R.L.U.) using a luminescence plate reader (Victor2, Perkin Elmer). Percent inhibition of virus entry was calculated from the R.L.U. values using the following formula: 100-[(R.L.U. (compound)−R.L.U. (minimum signal: cells no virus))/RLU (maximum signal: cells+virus)−R.L.U. (minimum signal: cells no virus))×100]. The anti-CD81 MAb JS-81 (BD Biosciences, San Jose, Calif.) was added to the HCVpp and Hep3B cells as a reference inhibitor.

Cloning Infectious HCV E1/E2 Envelope Glycoproteins from Patient Sera for the Production of HCVpp of Different Genotypes

Genotype specific primers and RT-Nested-PCR were used to amplify the E1/E2 gene from sera of individuals infected with different HCV 1a genotypes, or HCV 1b genotype, as described elsewhere (D. Lavillette et al., 2005). Briefly, viral RNA was isolated from 150 μL of infected patient serum using the QlAamp Viral RNA mini Kit (QIAGEN). Viral RNA was then reverse transcribed using the SuperScript™ III First-Strand Synthesis System for RT-PCR (Invitrogen). The resulting DNA served as template for a first round of amplification with genotype-specific outer primers followed by a second round of amplification with genotype-specific inner primers. The forward inner primer contained a 5′ CACC sequence to allow directional cloning of nucleic acid encoding HCV E1/E2 into the pcDNA3.1 TOPO vector (Invitrogen). Both rounds of amplification were performed with the high fidelity Platinum Pfx DNA polymerase (Invitrogen).

### High Throughput Screening Assay (HTS) for Inhibitors of HCVpp Entry

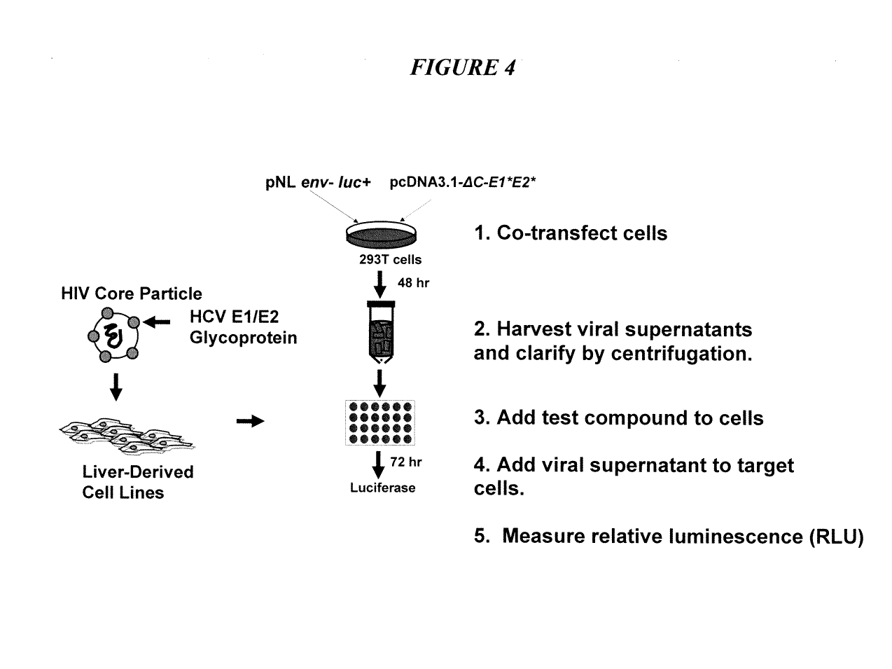

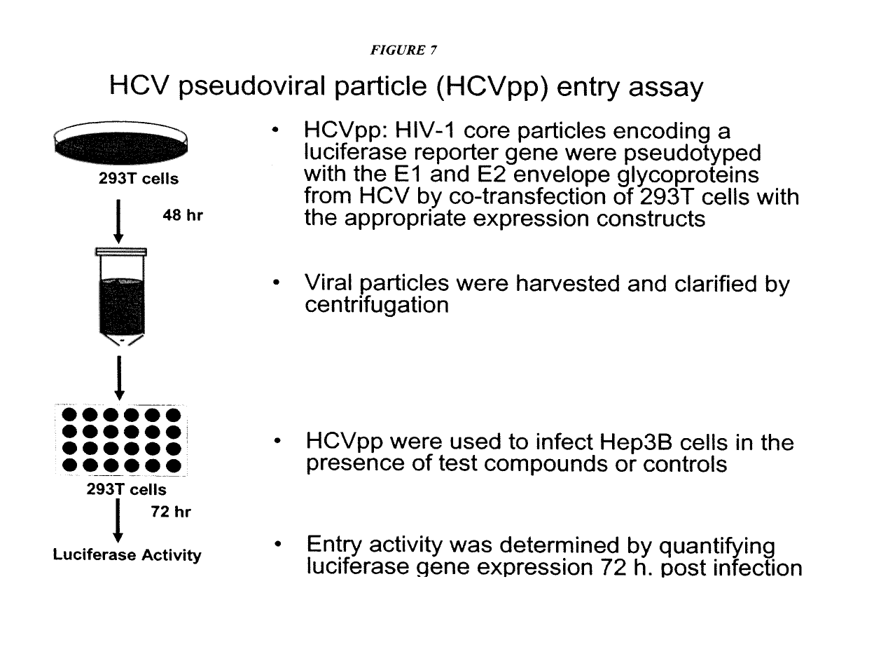

A schematic representation of the HIS assay is shown in FIGS. 4 and 7. Compounds were tested for their ability to inhibit HCVpp entry into the human hepatoma cell line Hep3B.

HCVpp were generated as described above. HCVpp-containing supernatants were stored at −80° C. and then thawed at 25° C. for thirty minutes prior to use in the inhibition of infection assay. Equal volumes (20 μl) of HCVpp and Hep3B cells (ATCC), (2×10³ cells/well), were plated in solid white 384-well plates (Perkin Elmer) in DMEM/2% FCS. Test compounds at 5 μM concentration in 0.5% DMSO (final) or control samples (5 μl) (DMSO alone or JS-81) were added to the HCVpp and Hep3B cells. After incubating the plates at 37° C. for 3 days, medium was removed from the wells and equal volumes of PBS and Bright-Glo (Promega, Madison Wis.) (25 μl) were added. Luciferase activity (Relative Light Units, R.L.U.) was measured by a luminescence plate reader (Victor2, Perkin Elmer). Percent neutralization of entry was calculated from the R.L.U. values using the following formula: 100−[(R.L.U. (compound)−R.L.U. (minimum signal: cells no virus))/RLU (maximum signal: cells+virus)−R.L.U. (minimum signal: cells no virus))×100]. The formulas used for data analysis were:

Maximum signal=average RLU values from wells with HCVpp and cells (vehicle alone)

Minimum signal=average RLU values from wells with cells only (no HCVpp or sample)

% Inhibition of entry=100−[(R.L.U. (compound)−R.L.U. (minimum signal: cells no virus))/RLU (maximum signal: cells+virus)−R.L.U. (minimum signal: cells no virus))×100].

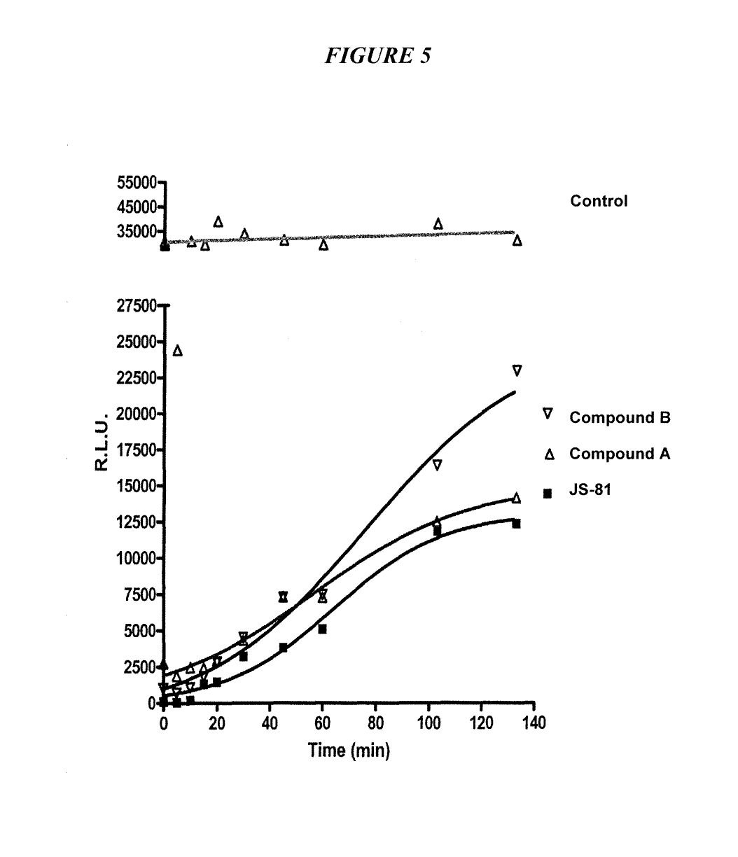

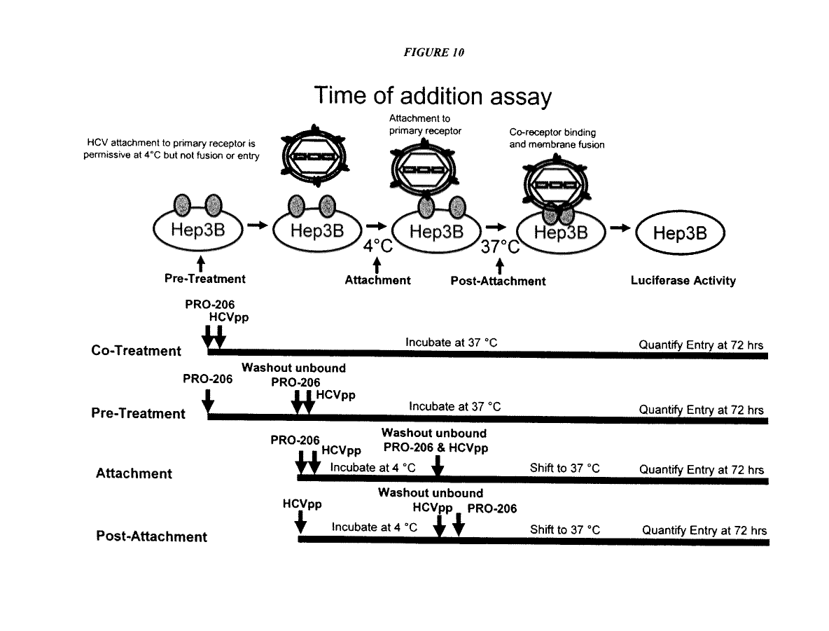

A time-of-addition assay was performed to evaluate inhibition of virus entry into cells and to assess the inhibitory effect of test compound addition as a function of time following exposure of susceptible target cells to HCVpp. (e.g., FIGS. 5, 10 and 11). The process of HCV entry can be subdivided into at least four distinct stages: 1) attachment to liver cells; 2) E2 binding to a co-receptor (e.g. CD81 and/or SR-BI); 3) rearrangement of tight junctions and co-localization of CD81/HCV complex with claudin-1; and 4) low pH induced E1 (or E2) mediated membrane fusion. The mechanism of action of an entry inhibitor can be inferred, in part, by establishing the time period that it is able to block entry of HCVpp into target cells. Time of addition studies were completed on compound PRO 206 (see Example 4) and showed that this compound potently inhibits a post-attachment step of HCV entry. The effect of anti-CD81 antibody JS-81 was used as a positive control. In FIG. 5, a non-active compound serves as a negative control.

### VSVpp Counterscreen

During HTS, test compounds were evaluated in parallel for inhibition of lentiviral particles pseudotyped with an envelope from an irrelevant virus, vesicular stomatitis virus (VSV-G) in order to eliminate from further consideration samples with non-specific activity. The resultant VSV pseudoparticles, VSVpp, used in screening potential HCV small molecule inhibitor compounds for specificity are well suited for this purposes because (a) VSV/G is unrelated to HCV E1/E2, (b) VSVpp possess a broad cellular tropism and efficiently infect Hep3B cells, and (c) VSVpp stocks can be produced at high titer and cryopreserved for later use in screening.

Activity screening results for exemplary compounds of the invention using the HCVpp assay are shown in Table 45.

TABLE 45
Relative Inhibition Effectiveness and Selectivity* for Blocking HCV Pseudovirus Particle (HCVpp) Entry into Hep3B Cells

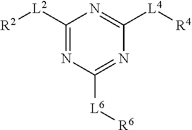

| L²R² | L⁴R⁴ | L⁶R⁶ | A¹ | A³ | A⁵ | Kᵢ (HCV)pp | SI** |
|---|---|---|---|---|---|---|---|
| 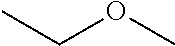 | 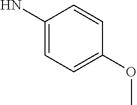 | 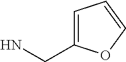 | N | N | N | +++ | +++ |
| 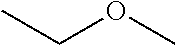 | 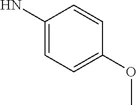 | 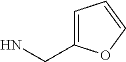 | CH | N | N | ++ | ++ |
| 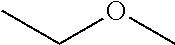 | 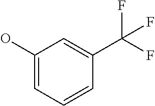 | 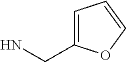 | N | N | N | + | + |
| 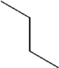 | 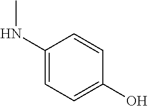 | 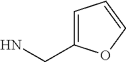 | N | N | N | +++ | ++ |
| 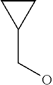 | 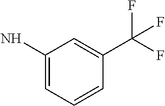 | 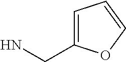 | N | N | N | +++ | ++ |
| —CN | 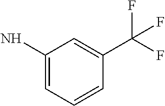 | 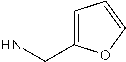 | N | N | N | ++ | + |
| 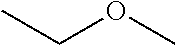 | 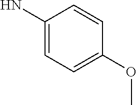 | 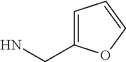 | N | N | N | ++ | ++ |
| —OH | 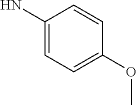 | 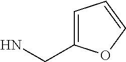 | N | N | N | + | + |
| 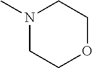 | 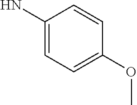 | 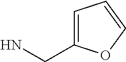 | N | N | N | ++ | + |
| 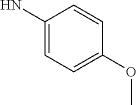 | 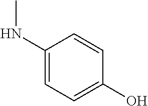 | 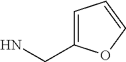 | N | N | N | +++ | +++ |
| 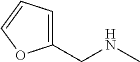 | 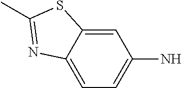 | 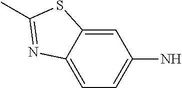 | N | N | N | +++ | + |
| 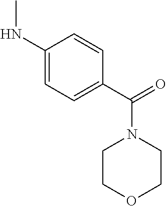 | 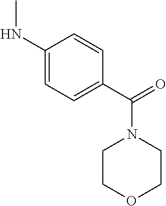 | 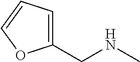 | N | N | N | ++ | + |
| 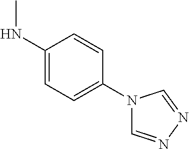 | 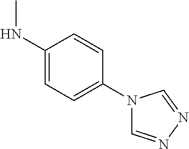 | 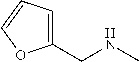 | N | N | N | +++ | +++ |
| 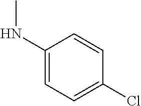 | 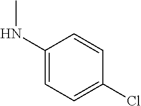 | 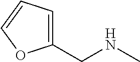 | N | N | N | ++ | ++ |
| 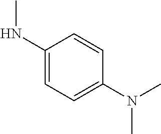 | 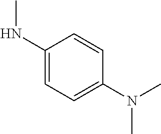 | 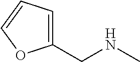 | N | N | N | +++ | +++ |
| 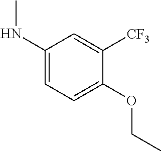 | 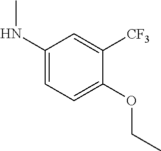 | 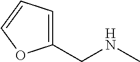 | N | N | N | ++ | ++ |
| 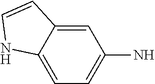 | 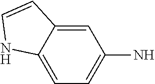 | 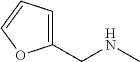 | N | N | N | +++ | ++ |
| 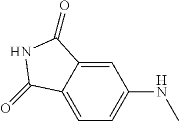 | 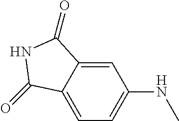 | 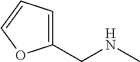 | N | N | N | +++ | +++ |
| —OEt | 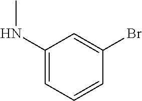 | 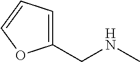 | N | N | N | + | + |
| —OEt | 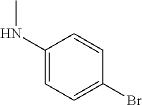 | 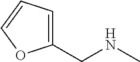 | N | N | N | +++ | ++ |
| —OEt | 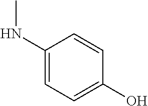 | 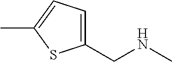 | N | N | N | ++ | +++ |
| —OEt | 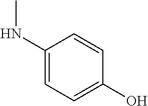 | 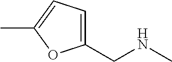 | N | N | N | +++ | +++ |
| —OEt | 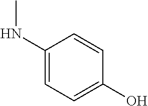 | 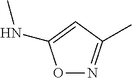 | N | N | N | +++ | +++ |
| —OEt | 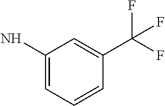 | 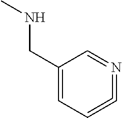 | N | N | N | +++ | +++ |
| —OEt | 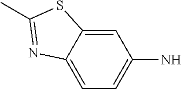 | 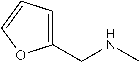 | N | N | N | +++ | ++ |
| —OEt | 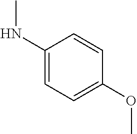 | 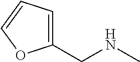 | N | CH | N | ++ | ++ |
| —CF₃ | 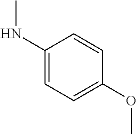 | 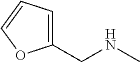 | N | CH | N | ++ | + |
| —OEt | 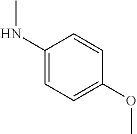 | 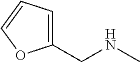 | CH | N | N | ++ | + |
| —CF₃ | 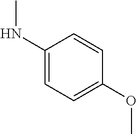 | 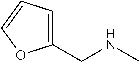 | CH | N | N | ++ | + |
| —CF₃ | 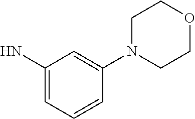 | 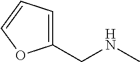 | N | CH | N | ++ | + |

*Specificity is determined with respect to cell entry inhibition of Vesicular Stomatitis Virus pseudo particles (see text)
**SI = selectivity Index = Kᵢ(VSV)pp/Kᵢ(HCV)pp. For Kᵢ (HCV)pp, the ranking scale is as follows: Kᵢ < 50 nanomolar, +++; 50 < Kᵢ < 1000 nanomolar, ++, Kᵢ > 1000 nanomolar, +. Pertaining to the Selectivity Index (SI), the ranking scale is as follows: SI < 10, +; 10 < SI < 100, ++; SI > 100, +++.

In the primary and subsequent HCVpp entry assays, compounds of the invention exhibited potent and selection inhibitory activity (EC₅₀s ranged from 28 nM to 151 nM). An antiviral selectivity index (SI) was calculated by dividing the EC₅₀, as determined in the VSVpp assay by the EC₅₀ determined in the HCVpp assay. In some cases, representative compounds exhibited 61 to 714-fold greater selectivity for HCVpp compared to VSVpp. The hit compounds also demonstrated excellent selectivity when assayed for their activity against irrelevant envelopes including those from MLV and HIV. Representative compounds were assayed for cytotoxicity against Hep3B cells under conditions that were identical to the primary HCVpp entry assay. A therapeutic window was calculated by dividing the CC₅₀ observed in Hep3B cells by the EC₅₀ determined in the HCVpp assay. Hit compounds exhibited low cytotoxicity, with a calculated therapeutic window of 84 to 2,579-fold.

### Inhibition of HCVpp of Different Genotypes.

Compounds were tested for their ability to inhibit infection of susceptible cells by various and representative genotype 1 envelopes of HCV in a pseudovirus infectivity assay employing HCVpp as described. The anti-CD81 JS-81 monoclonal antibody (BD Biosciences, San Jose, Calif.) was used as a reference standard and was tested in parallel with the compounds for inhibition of HCVpp (virus) entry into cells.

Compounds were diluted in DMSO and were added to Hep3B cells immediately prior to the addition of HCVpp derived from different genotype isolates, e.g., genotype 1a (HCV strain H77) or genotype 1b. Plates were incubated for 72 hours prior to measurement of luciferase activity.

In accordance with the invention, compounds generally exhibited virus infection inhibitory activity against HCV of genotype 1a and 1b isolates tested, with a median EC₅₀ value in the range of 0.00001-3 μM.

### EC₅₀ Determination

Primary HTS is a single point measurement that calculates % inhibition only at a 5 micromolar drug concentration. For dose response and EC₅₀ determination, compounds were subjected to 10 serial 0.5 log₁₀ dilutions in DMSO. The diluted compounds were added to target cells as described which were then infected with HCVpp. After 72 hours viral entry as measured by luciferase activity was determined using a 4-parameter curve fitting program within a customized template in Activity Base (IDBS).

Development of HCVpp Assays Based on Diverse E1/E2 Envelopes Obtained from HCV Infected Patient Sera

To examine the breadth of antiviral activity of potent and selective hits, multiple infectious E1/E2 variants were cloned from a given HCV+serum. Utilizing a modified high-throughput, miniaturized version of an E1/E2 cloning strategy, HCV E1/E2 gene sequences representing amino acids 170 to 746 were amplified by nested RT-PCR as described. (Lavillette, D. et al., 2005, Hepatology, 41:265-274). Purified viral RNA isolated from 150 μL of HCV⁺ patient serum was subjected to reverse transcription using the SuperScript™ III First-Strand Synthesis System (Invitrogen). The resulting cDNA served as a template for a nested PCR amplification of the E1/E2 envelope with genotype-specific primer pairs as described previously. The 1.7 kb PCR product was gel purified using the QIAquick Gel extraction kit (Qiagen) and ligated into the pcDNA3.1-TOPO expression vector (Invitrogen). Multiple plasmid DNA clones encoding unique E1/E2 quasispecies were isolated from each of the patient sera and verified by DNA sequencing. Co-transfection of 293T cells and production of HCVpp were adapted to a 96-well format from previously described methods. Cleared viral supernatant (200 μL) was incubated with Hep3B target cells (2,000 cells/well) and luciferase activity was measured 72 hrs post-infection using BriteGlo reagent (Promega).

HCVpp-based assays were developed representing 15 authentic HCV viral envelopes from different patients infected with genotype 1. Multiple quasispecies were identified within each serum. A relatively low percentage of the isolated E1/E2 expression constructs were fusogenic when expressed on HCVpp. Quasispecies were verified to be unique by sequencing and typically differed from one another at 5-10 positions within the 130-amino-acid HVR1 region of E2. Transfection conditions for each envelope were optimized and individual HCVpp assays were validated with JS-81. The genotype spectrum panel was used to evaluate the potency and breadth of the antiviral activity of compounds of the invention.

### Cytotoxicity Assay for Testing HCV Inhibitor Compounds

Profiling antiviral compounds in cytotoxicity assays is a critical step to determine that the antiviral activity observed in the primary assay is not due to overt cytotoxicity. To profile the HCV inhibitor compounds of the invention, a panel of cytotoxicity assays was developed based on nine cell lines (Hep3B, Huh-7, Huh-7.5.1, Ramos, Daudi, Jurkat, 293T, HeLa, and U87), as well as primary hepatocytes grown in culture. All cytotoxicity assays were benchmarked against the pan-kinase inhibitor staurosporine. The cytotoxic concentration required to reduce cell viability by 50% (CC₅₀) for staurosporine was determined concurrently in all cytotoxicity assays and served as a reference compound for assay validation and data acceptance.

The compounds of the invention were profiled in cytotoxicity assays against Hep3B cells. These assays were carried out for 72 hours under conditions that were identical to those used in the HCVpp primary assay. Briefly, cells were plated in 384-well plates in the presence or absence of serially diluted compounds. After 72 hours, cell proliferation was determined using the CellTiter-Glo assay (Promega). In this assay, luminescence is a direct read-out of the number of viable cells in the microplate. Dose-response curves were generated and the CC was determined from the curve for each cell line. A cytotoxicity-based therapeutic index (TI=CC₅₀/EC₅₀) was determined for all compounds. Compounds with TI>10 were determined to have a high likelihood of being specific antivirals.

### BIAcore Based Binding Assays

In order to determine whether HCV inhibitor compounds of the invention bound to the E2 envelope of HCV, the binding of entry inhibitors to a truncated soluble form of the E2 glycoprotein (sE2) was examined by BIACore. Soluble E2 was immobilized to BIACore chips according to the manufacturer's instructions. A binding assay using the E2 bound chip was established and validated with PA-25, a mouse MAb that was generated against sE2 (positive control) and two negative controls: PA-1, a MAb that recognizes HIV-1 gp120 and an isotype control. Surface plasmon resonance was monitored during the flow of antibody controls or small-molecule inhibitors over immobilized sE2. As expected, PA-25 demonstrated strong binding to sE2 in the assay, however, representative small-molecule entry inhibitors did not demonstrate binding to E2 in the BIACore assay. As expected, the negative controls, PA-1 and the isotype control, did not exhibit specific binding to sE2 in the study.

### Example 2

### In Vivo Animal Model Suitable for Assessing HCV Inhibitor Compounds

An in vivo model of HCV infection may be employed for assessing the activity of compounds of the invention. Such a model involves the use of SCID mice carrying a plasminogen activator transgene under control of the albumin promoter (Alb-uPA), (Kneteman, N. M. et al., 2003; Mercer, D. F. et al., 2001; Kneteman, N. M. et al., 2005; Meuleman, P. at al., 2005; Hiraga, N. et al., 2007, FEBS Letters, 581:1983-1987). SCID mice are homozygous for a mutation that impairs the recombination of gene segments (V, D and J) that code for the variable (antigen-binding) regions of antigen receptors (Ig molecules) in lymphocytes. Such mice lack mature, functional lymphocytes from both the T and B cell lineages. The transgene directs overproduction of urokinase in the liver resulting in accelerated death of hepatocytes. Engraftment of human liver cells into these mice rescues the animals from liver failure.

The integrity of the human liver tissue grafts is monitored by assessing human alpha-1 antitrypsin (hAAT). The human liver graft can be infected with HCV in vivo. SCID/Alb-uPA mice engrafted with human liver tissue are infected by inoculation of HCV positive human serum. Following the establishment of infection, viral load in the animals ranges from 10⁴-10⁷ RNA copies/ml (based on Amplicor test, Roche) and infection can be maintained in these animals for up to 4 months. In this system, the animals are treated with a candidate molecule (e.g., an HCV inhibitor, such as an HCV entry inhibitor compound of this invention) before and/or after exposure to HCV in order to examine the therapeutic effectiveness of the inhibitor.

### Example 3

### Assessment of Anti-Hepatitis C Virus Activity in an HCV Cell Culture (HCVcc) Assay

Prior to the development of the HCVcc assay, patient sera were utilized to infect primary hepatocytes or hepatoma cell lines, typically resulting in low level and poorly reproducible viral replication. Substantial efforts were made to develop systems that reliably and robustly produced infectious HCV in vitro. Such systems using an HCV clone (JFH1, genotype 2) derived from a Japanese patient with fulminant hepatitis were reported several years ago. (Kato, T. et al., 2003, Gastroenterology, 125:1808-1817; Lindenbach, B. D. et al., 2005, Science, 309:623-626; Wakita, T. et al., 2005, Nat Med, 11:791-796; Zhong, J. et al., 2005, Proc Natl Acad Sci USA, 102:9294-9299). The subgenomic (replicon) clone of this genotype 2 isolate replicates efficiently in cell culture in the absence of adaptive mutations typically associated with HCV replicon sequences. (Krieger, N. et al., 2001, J. Virol., 75:4614-24). Full-length clones, containing either the complete JFH1 consensus sequence or JFH1 nonstructural proteins in association with the core-through-NS2 regions of another genotype 2 clone, J6, demonstrated robust replication in Huh-7-derived cell lines. Cells transfected with the cloned viral genome secreted HCV particles that were infectious in vitro and in chimpanzees. HCVcc could be inhibited with IFN-α and by small-molecule inhibitors of the HCV serine protease NS3. HCVcc could be propagated in vitro, particularly on Huh-7 sublines that had been transfected with and then cured of HCV replicons.

HCVcc enabled the study of entry by authentic HCV in vitro, and the findings have been remarkably convergent with those obtained using HCVpp. HCVcc entry is pH dependent and is restricted to CD81-positive liver cells. CD81-negative HepG2 cells become permissive to HCVcc infection when modified to express CD81. HCVcc infection is inhibited by MAbs to CD81 and to recombinant forms of the large extracellular loop of CD81. Likewise, MAbs directed against SR-BI or tagged claudin-1 also inhibit HCVcc entry. HCVcc infection is inhibited by sera from HCV-infected individuals but not by normal human sera. Infection is inhibited by MAbs directed against the E1 and E2 envelope glycoproteins. The findings corroborate those obtained using HCVpp and support the view that HCVpp accurately recapitulate the essential biology of HCV entry.

In addition to studying the HCV biology and assessing the antiviral activity of HCV drugs, the HCVcc system has also been used successfully in drug resistance studies. The JFH1 HCVcc system was used to develop drug resistance against the protease inhibitor BILN-2061 (Cheng, G. et al., 2008, Efficient In Vitro Selection of Drug-Resistant Mutants Using the HCV Infection System. 15th International Symposium on Hepatitis C Virus and Related Viruses Oct. 5-9, 2008 San Antonio, Tex.). The in vitro resistance profile for BILN-2061 correlated with viral resistance patterns obtained in the replicon with BILN-2061 and in the clinic with other protease inhibitors. The recent availability of chimeric full-length constructs containing the nonstructural proteins of JFH-1 and the structural proteins of genotype 1 clones such as H77C, J4 or Coni have provided model, chimeric HCVcc systems in which to determine antiviral activity, mechanism of action and determinants of drug resistance for inhibitors of HCV entry using genotype 1 HCV strains.

To examine the anti-HCV activity of the small molecule triazine compounds according to the present invention, an HCV cell culture (HCVcc) assay (or model) was performed using an HCV susceptible cell line, such as Huh7.5, infected with a genetically engineered HCV of genotype 2a or genotype 1a/2a. The HCVcc system affords the opportunity to study the ability of compounds of the invention to inhibit HCV of different genotypes from infecting target cells, e.g., in a manner that is more akin to in vivo virus infection conditions. More specifically, testing the inhibition of HCV entry and infection of Huh 7.5 cells by the compounds of the invention in the HCVcc assay involved the following parameters:

Cytotoxicity. Cytotoxicity was evaluated with 9 concentrations per compound (drug) at 4-fold dilutions (if not otherwise specified) in DMEM+0.5% DMSO. Compounds at the various concentrations were added to Huh7.5 cell monolayers in replicates of 4 and incubated for 72 hours. Thereafter, the cytotoxic effects were determined by quantification of ATP levels using a CellTiter-Glo® Luminescent Cell Viability Assay kit (Promega Corporation) according to the manufacturer's instructions.

Dose Response. A dose response assay was performed by infecting naíve Huh7.5 cells with reporter virus, with or without adaptive mutations, in replicates of 4, unless the assay signal and/or variability required 8 replicates. For infection, the diluted compounds were added to the respective wells, using the same dilution series as described above for cytotoxicity. At 72 hours post-infection (p.i.), the luciferase levels of the cell lysates were determined using the Renilla Luciferase Assay System (Promega) according to the supplier's instructions. The cell supernatants were stored at −70° C. for TCID50 analysis. As a positive control, the anti-CD81 antibody JS-81 was assayed in parallel with test compounds.

Compound/Drug Preparation. The HCVcc assays were performed in 0.5% DMSO, i.e., for consistency with primary screening assays that were performed on the compounds. Huh7.5 and Huh-7 cells were compatible with this level of DMSO. Prior to analysis, plates were prepared containing compounds at the determined serial dilutions.

Study Design. For dose response, Huh7.5 cells were seeded into 96-well plates at a density of 6×10³ cells/well. The anti-HCV small molecule compounds to be analyzed were at 10 uL of 200× concentrated compound serially diluted in 100% DMSO in 96-well plates. Compounds were diluted stepwise into HCVcc medium containing HCV of genotype 1a/2a, e.g., H77/JFH-1, as follows: Step 1: The dilution plate was prepared by adding 237.5 uL of H77/JFH-1 supernatant into a 96-well plate. For the minus virus control, 237.5 uL of assay medium only was added. Step 2: 90 uL of viral supernatant was added to the plate containing test compounds to result in a 1:10 dilution. DMSO concentration was at 10% and compound concentration was 20×. 90 uL of assay medium only was added to the minus virus control. After each addition of virus, the compound was carefully mixed with virus and was rapidly pipetted up and down with a multi-channel pipette. Step 3: 12.5 uL of diluted compound was transferred to the dilution plate prepared in Step 1 above, resulting in a 1:20 dilution. The final concentration of DMSO was 0.5%. The final concentration of compound was 1×. After each transfer, the compound was carefully mixed with virus and rapidly pipetted up and down with the multi-channel pipette. Thereafter, medium was completely removed from the Huh-7.5 cells and replaced with 200 uL of virus+/−compound from Step 3 above. Luciferase levels were determined at 72 hours p.i.

For cytotoxicity analysis, Huh7.5 cells were seeded into 96-well plates at a density of 6×10³ cells/well. The following day, the compounds were diluted to their final concentrations as described above. Compounds were added to the 96-well plate in 200 μl final volume (a larger volume is preferred for screening to prevent evaporation) in replicates of 8, to mimic the dose response assay without virus addition. After 72 hours the ATP levels of each well were determined. The Cell Titer Glo® assay system was used for cytotoxicity testing, as per the standard protocol. For dose response testing, supernatants were collected; luciferase signal was determined on the cell lysates; and infectivity testing (TCID50) was performed on the supernatant.

### Example 4

The novel compounds discovered in accordance with the present invention were evaluated in a number of assays to assess their activity and function as HCV entry inhibitors. This example relates to discovery, findings and experiments involving the compounds of the invention, and more particularly to exemplified compound PRO206 whose properties were studied as described in this Example.

Background: Combinations of specific anti-viral drugs, with complementary mechanisms of action and determinants of resistance offer the potential to improve sustained virologic response (SVR) rates with reduced toxicity for HCV infected patients. To date, most drug discovery efforts have focused on two viral enzyme targets of HCV: the NS3/4A serine protease and the NS5B RNA-dependent RNA polymerase. Viral entry represents an additional therapeutic target that has been validated clinically for other pathogenic viruses.

Methods: To discover novel inhibitors of HCV entry, we established a robust high-throughput screen based on HCV pseudo-particle (HCVpp) technology. A hit-finding campaign was conducted using a proprietary 370,000-member library of drug-like compounds. Hit series were optimized for potency, selectivity and pharmacokinetic (PK) properties through multiple iterations of medicinal chemistry.

Results: The primary screen yielded multiple drug-like chemotypes. Following optimization, lead compounds demonstrated nanomolar activity against a panel of genotype 1 viruses in the HCVpp assay. Selective entry inhibitors exhibited minimal cytotoxicity and limited activity against retroviral particles psuedotyped with the envelope glycoproteins from multiple unrelated viruses. Finally, our lead compounds demonstrated favorable in vitro ADME (absorption/distribution/metabolism/excretion) properties and oral bioavailability in animals.

Conclusions: Drug-like compounds were identified that selectively inhibited HCV entry with low nanomolar potency. These compounds demonstrated favorable oral PK in animals and warrant further exploration as potential therapies from a novel class of drugs to treat HCV infection.

In connection with the discovery and optimization of one of the compounds of the invention, designated PRO206, a library of diversified small molecule compounds was screened for their ability to block entry of HCVpp into Hep3B cells. Hits were counter-screened against viral particles pseudotyped with the envelope G-glycoprotein from vesicular stomatitis virus VSV (VSVpp). This screening strategy yielded multiple chemotype inhibitors of HCV entry. Chemically tractable hit series with established structure activity relationships (SAR) and good drug-like properties were further optimized through a medicinal chemistry effort. Systematic chemical optimization to improve potency, selectivity, drug-like properties and pharmacokinetics led to the discovery of PRO206.

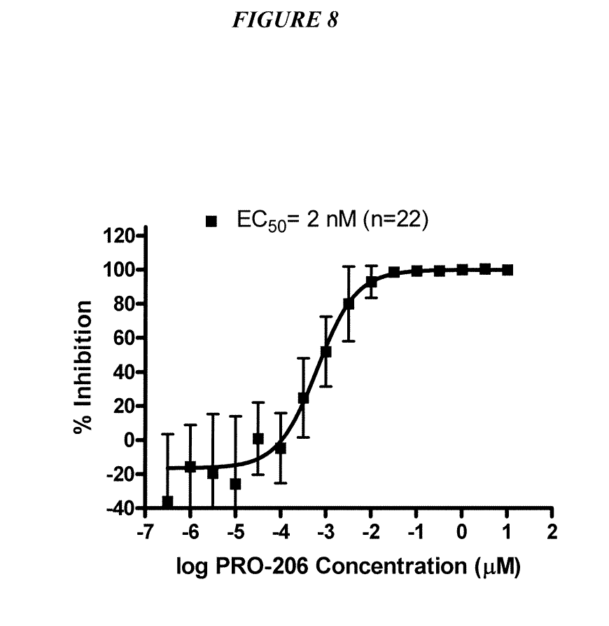

PRO206 is a broadly potent and selective inhibitor of HCV entry: With regard to the spectrum of activity of PRO206, unique envelope sequences were obtained from sera of patients infected with HCV. HCVpp assays derived from cloned E1/E2 envelopes were individually optimized and validated with the anti-CD81 mAb JS-81. For these experiments, PRO206 was subjected to ten 0.5 log₁₀ serial dilutions in DMSO. Various concentrations of PRO206 were assayed for their ability to block entry of H77 HCVpp into Hep3B cells as described above. The dose response curve shown in FIG. 8 represents a composite of 22 independent assays. PRO206 exhibited potent activity in the primary HCVpp entry assay, with an EC₅₀=2 nM. In contrast to the potent inhibitory activity against HCVpp exhibited by PRO206, PRO206 did not significantly inhibit the entry of pseudoviral particles complemented with the envelope glycoproteins from irrelevant viruses including VSV, MLV and HIV. The EC₅₀ for PRO206 acting against each envelope is summarized in Table 46. PRO206 displayed >17.000-fold selectivity for HCVpp over VSVpp or MLVpp. Likewise, PRO206 demonstrated >50.000-fold selectivity for HCVpp over HIVpp.

TABLE 46
Antiviral Selectivity of PRO 206

| Viral | EC50 |
|---|---|
| Envelope | (μM) |
| HCV (H77) | 0.002 |
| VSV | 39 |
| MLV | 34 |
| HIV_{SF162} | >100 |

To determine the potency and breadth of activity against multiple envelopes, PRO 206 was profiled against a panel of genotype 1-based HCVpp assays. The unique fusogenic envelopes were derived from HCV patient sera as described above. All HCVpp particles were produced by co-transfection of 293T cells with the HIV-1 luciferase reporter and the respective expression construct encoding unique E1/E2 envelope glycoproteins obtained from HCV⁺ patient sera. Viral titers were determined by serial dilution of pseudovirus stocks followed by infection of Hep3B cells. After 72 hrs, viral entry was quantified by measuring luciferase activity. Pseudoviral stocks were normalized to ˜100,000 RLU and used in profiling assays to determine the potency of PRO206 against each envelope. The results from multiple dose down experiments, summarized in Table 47, demonstrate that PRO206 potently inhibited viral entry mediated by 13 out of 16 genotype 1 envelopes. A median EC₅₀=11 nM was calculated across all of the envelopes in the panel.

TABLE 47
Spectrum of activity of PRO 206 against
envelopes isolated from HCV+ patient sera

| | EC₅₀ | |
|---|---|---|
| Envelope | (μM) | N |
| EG | 0.001 | 5 |
| H77 | 0.002 | 22 |
| MZ | 0.002 | 3 |
| NC | 0.002 | 3 |
| MA2 | 0.006 | 7 |
| CT | 0.007 | 6 |
| WF | 0.008 | 3 |
| MA6 | 0.009 | 4 |
| ML | 0.012 | 1 |
| DR | 0.041 | 4 |
| HPTK | 0.050 | 3 |
| JS | 0.183 | 2 |
| RA | 0.214 | 3 |
| C1 | 0.817 | 6 |
| VP | >3.8 | 6 |
| PG8 | >10 | 3 |

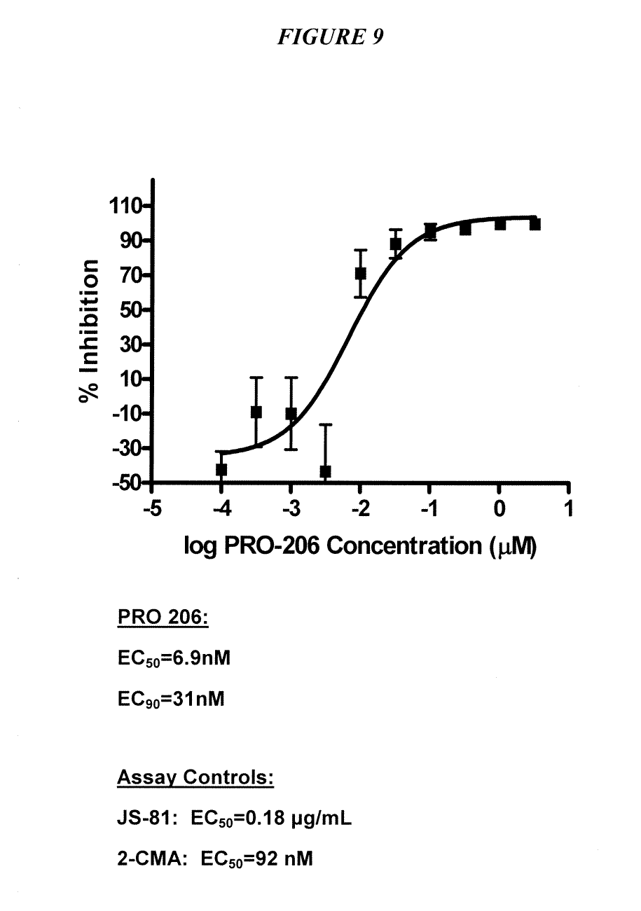

PRO206 demonstrates potent antiviral efficacy in the HCV cell culture model: There is currently a dearth of robust screening models for demonstrating antiviral efficacy in vivo. The HCV infected chimpanzee and the SCID/uPA mouse model of HCV infection are two systems that have been used to demonstrate preclinical proof-of-concept for inhibitors of the NS3 protease and the NS5B polymerase (Chen, C. M., et al., 2007, Antimicrob Agents Chemother., 51:4290-4296; Lanford, R. E. et al., 1994, Virology, 202:606-614; Mercer, D. F. et al., 2001, Nat Med, 7:927-33). However, such animal models are characterized by poor accessibility and restrictive costs. Moreover, neither model has been adequately validated for the evaluation of small-molecule inhibitors of HCV entry. PRO206 was tested in an H77/JFH-1 HCVcc system (Dr. Charles Rice, The Rockefeller University) which serves as the major determinant of antiviral efficacy for genotype 1 HCV in lieu of an in vivo screening model. A chimeric HCVcc RNA, encoding the non-structural proteins from H77 (genotype 1) fused to the structural proteins from JFH-1 (genotype 2) was electroporated into naïve Huh-7.5 cells. At 3 days post-transfection, viral supernatants were harvested and used to infect naïve Huh-7.5 cells in the presence of various concentrations of PRO206. Renilla luciferase activity was measured 72 hours post-infection. PRO206 demonstrated potent antiviral activity against H77/JFH-1 HCVcc in culture with an EC₅₀=6.9 nM and an EC₉₀=31 nM (shown in FIG. 9). PRO206 did not exhibit cytotoxic effects in Huh-7.5 cells at the highest concentration examined (3 μM).

PRO206 has a large cytotoxicity-based therapeutic index (T.I.): The cytotoxic potential of PRO206 was determined by evaluating the compound's effect on the proliferation of nine mammalian cell lines, representing multiple cell and tissue types. The time period used for the proliferation assays was 72 hours, which allows for at least two cell doublings and provides a rigorous assessment of the effect of PRO206 on dividing cells. The CC₅₀ for PRO206 in all cell lines was determined from dose response curves and ranged from 15 μM to 100 μM (the highest drug concentration tested). The cytotoxicity-based therapeutic window for each cell line is summarized in Table 48. Using the mammalian cell proliferation CC₅₀, and the median EC₅₀=11 nM for PRO206 acting against multiple envelopes, the TI for PRO206 was calculated to be >2,000 in all cell lines tested. In non-replicating primary hepatocytes, PRO206 did not affect cell viability at the highest concentration tested (100 μM), as determined in the CellTiter Glo assay. Importantly, the window between antiviral activity and cytotoxicity in Hep3B cells was >2.500-fold, further demonstrating that PRO206 is a highly selective inhibitor of HCV entry.

TABLE 48
PRO 206 demonstrates low cytotoxicity against
a panel of cell lines and primary hepatocytes

| Cell | Tissue | Morphology | T.I. (X) |
|---|---|---|---|
| Hep3B | Liver | Epithelial | 2,583 |
| Huh-7 | Liver | Epithelial | 1,575 |
| Huh-7.5.1 | Liver | Epithelial | >8,333 |
| 293T | Kidney | Epithelial | >8,333 |
| HeLa | Cervix | Epithelial | >8,333 |
| Daudi | Blood | Lymphoblast | 1,467 |
| Jurkat | Blood | Lymphoblast | 1,567 |
| Ramos | Blood | Lymphoblast | 1,283 |
| U87 | Brain | Epithelial | 1,975 |
| Primary | Liver | Epithelial | >8,333 |
| Hepatocyte | | | |

In Table 48, Cytotoxicity-based T.I. (Therapeutic Index) = CC₅₀/EC₅₀;
EC₅₀ = 12 nM is based upon the median EC₅₀ of 17 envelopes.

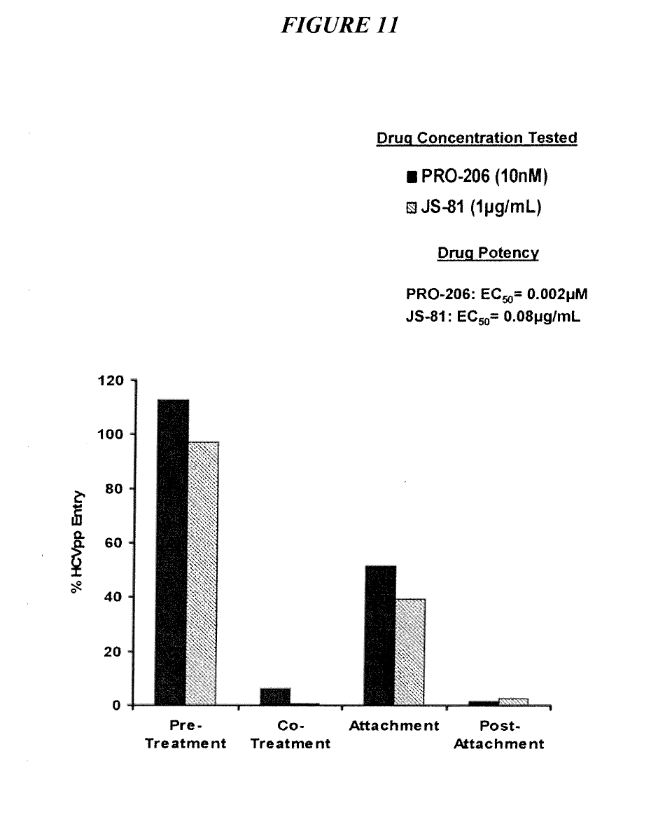

The mechanism of action of PRO206 is consistent with a post-attachment entry inhibitor: To determine whether PRO206 binds to a primary receptor and blocks attachment of HCVpp to target cells, inhibition of binding and entry experiments were performed. The assay takes advantage of the finding that HCVpp attachment, but not fusion, can occur at 4° C. To determine if PRO206 binds to the primary receptor on the cell surface, Hep3B cells were incubated with PRO206 or JS-81 (Pre-Treatment) at 4° C. After washing away unbound compound or MAb with PBS, HCVpp were added to Hep3B cells and the cultures were shifted to 37° C. Viral entry was quantified after 72 hours by measuring luciferase activity. Pre-treatment of Hep3B cells with PRO206 or JS-81 resulted in no inhibition of HCV entry. (FIG. 11). In contrast, when compounds were added to Hep3B cells in the standard assay format (Co-Treatment) potent inhibition of entry was observed. To further examine inhibition at the attachment phase, HCVpp were allowed to attach to Hep3B cells at 4° C. in the presence of PRO206. After 2 hours' incubation, unbound compound and HCVpp were washed out and the cultures were shifted to 37° C. to allow entry to proceed. Under these conditions, only weak inhibition (˜2-fold) was exhibited by JS-81 or PRO206. These results suggest that PRO206 does not inhibit the attachment of HCVpp to target cells. A contrasting result was obtained when PRO206 was added to infection assays after attachment of HCVpp to Hep3B. After a 2 hour incubation at 4° C. to allow HCVpp attachment, unbound virus was washed out with PBS and PRO206 or JS-81 was added to the cultures, which then were shifted to 37° C. to allow entry to take place. Potent inhibition of entry was observed (>95%) when either JS-81 or PRO206 was added post-attachment. As expected, addition of JS-81 or PRO206 four hours post-entry demonstrated only weak inhibitory activity. Taken together, the results demonstrate that PRO206 functions as a post-attachment entry inhibitor with a profile that is similar to the profile published for the anti-CD81 MAb, JS-81. (Cormier, E. G. et al., 2004, Proc Natl Aced Sci USA, 101:7270-7274; Evans, M. J. et al., 2007, Nature, 446:801-805).

As outlined and further described below, the in vitro ADME (Absorption, Disposition, Metabolism, Excretion) properties of PRO206 were found to be highly favorable. The ADME and drug-like profile of PRO206 involved the parameters of Drug-likeness, Cytochrome P450 inhibition, Caco-2 permeability and Human microsomal stability, with the following results:
Drug-likeness: Lipinski's rules
No major violations
MW<550
clogP<5
H-bond donors <5
H-bond acceptors <10
Cytochrome P450 Inhibition
No significant inhibition observed at drug concentrations >500× the EC₅₀ for PRO 206 (determined in the primary assay)
CYP3A4=0%
CYP2D6=7%
CYP2C19=15%
CYP2C9=7%
CYP1A2=0%
Caco2 permeability
High Caco-2 permeability suggests that PRO206 will be well absorbed in vivo
Human microsomal stability
No metabolism observed
Potential for long in vivo half life

PRO206 exhibits favorable ADME and drug-like properties: PRO206 exhibits excellent drug-like properties with no major violations of Lipinski's rules (Lipinski, C. A. et al., 2001, Advanced Drug Delivery Reviews, 46:3-20), (i.e. MW˜500, clogP<5, H-bond acceptors <10, H-bond donors<5, Rotatable bonds<10). To assess metabolic stability, PRO206 was incubated with pooled human liver microsomes in the presence of an NADPH-generating system at 37° C. for 45 minutes. Unchanged PRO206 was quantified by LC/MS. In this assay, no measurable degradation of PRO206 was observed. To ascertain the potential of PRO206 to inhibit several major isoforms of Cytochrome P450, PRO206 (1 μM) was incubated with human recombinant CYP2C9, CYP2C19, CYP1A2, CYP2D6 and CYP3A4 in the presence of the corresponding fluorescent substrates and an NADPH regeneration system. PRO206 did not significantly inhibit any of the five tested CYP isoforms (% inhibition≦15%). Taken together, these results indicate that PRO206 has a low potential for clinically meaningful drug-drug interactions.

To examine the absorption properties of the compound, PRO206 was assessed in a Caco2 permeability assay. The absorption potential of PRO206 as determined on Caco2 monolayers was found to be high, predicting that the compound will be well absorbed in humans. It is noted that the favorable in vitro ADME properties, obtained from multiple ADME screens, are consistent with the favorable PK properties observed in animals, as described below.

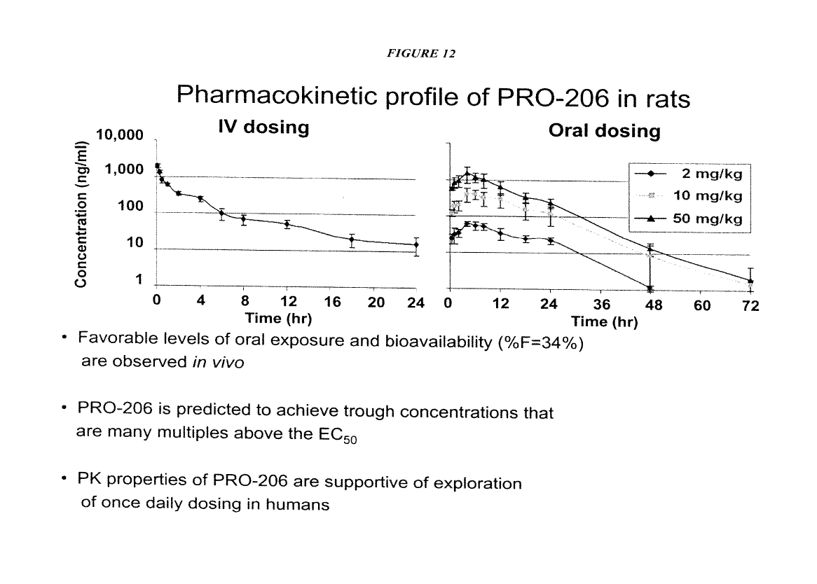

PRO206 exhibits a favorable pharmacokinetic (PK) profile in rats: The PK profile of PRO206 was evaluated in rats via IV dosing and oral dosing. (FIG. 12). The pharmacokinetics of PRO206 were measured following a single 2 mg/kg IV dose and single ascending oral doses (2, 10 and 50 mg/kg) to rats. Both IV and oral formulation were Solutol® hydroxylstearate 15-based (BASF). Solutol® HS15 is a non-ionic solubilizing agent consisting of mono- and di-esters of 12-hydroxystearic acid (lipophilic components) and ˜30% of free polyethylene glycol (a hydrophilic component). The resulting plasma concentration-time profiles are depicted in FIG. 12.

Following a 2 mg/kg IV dose, the systemic clearance of PRO206 averaged 615 mL/h/kg. Volume of distribution at steady state (V_{dss}) averaged 3415 mL/kg, indicating a wide distribution of PRO206 to the peripheral compartments. Plasma concentrations decayed with an average terminal half-life of 6.7 hours. After oral doses of 2, 10, and 50 mg/kg, Cₘₐₓ and AUC_{inf} increased approximately in proportion with increasing dose. Average Cₘₐₓ values were 61.9 ng/mL at 2 mg/kg, 445 ng/mL at 10 mg/kg, and 1482 ng/mL at 50 mg/kg. The rate of absorption was also apparently dose-independent, with Tₘₐₓ values averaging 5.2 h at 2 mg/kg, 6.4 h at 10 mg/kg, and 4.0 h at 50 mg/kg. The average terminal half-life was 6.6-6.7 h at each PO dose. Bioavailability (% F) of the oral dose was 34.0% at 2 mg/kg, 35.6% at 10 mg/kg, and 23.8% at 50 mg/kg. The results of the PK analysis showed that favorable in vivo levels of oral exposure and bioavailability (% F=34%) of PRO206. PRO206 is predicted to achieve trough concentrations that are many multiples above the EC₅₀. Thus, the PK properties of PRO206 support an oral dosing of this compound, and more particularly, an exploration of a once daily dosing regimen in humans.

PRO206 exhibits a favorable safety profile: PRO206 was well tolerated following a single oral dose of 2, 10 and 50 mg/kg to rats. During the 72-hour post-dose observation period, there were no drug-related abnormalities noted. To enhance the safety assessment, necropsies were conducted 72 hours following a single oral dose. Necropsies indicated normal appearance of all internal organs in all dose groups. In addition to the in vivo findings described above, PRO206 was assessed in an additional in vitro cardiac safety screen. The hERG potassium channel is a voltage-gated ion channel found in the heart. It is essential for cardiac re-polarization, yet many pharmacological agents can inhibit the hERG current and cause QTc prolongation leading to arrhythmia and death. The potential of PRO206 to inhibit hERG channel was tested in CHO cells stably transfected with hERG. The measurements were performed using a single-cell patch-clamp apparatus. The results showed that PRO206 did not demonstrate hERG activity at concentrations ranging to 10 μM.

The potential off-target effects of PRO206 were evaluated by measuring PRO 206 interactions with a diverse panel of 69 receptors and 16 enzymes. The interactions with the human recombinant receptors were evaluated in the receptor binding assays in the presence of the corresponding receptor-specific radio-labeled ligands, while interactions with therapeutically relevant enzymes were evaluated in enzyme inhibition assays performed in the presence of enzyme-specific substrates. PRO206 did not cause appreciable inhibition of any of the studied receptors or enzymes (% inhibition≦18% for all), indicating a low potential for off-target interactions.

Genetic toxicity of PRO206 was assessed in a bacterial reverse mutation test (AMES assay) conducted in two strains of Salmonella typhimurium (TA98 and TA 100) in the presence or absence of rat liver S9 (Ames, B. N. et al., 1973, Proc Natl Acad Sci USA, 70:2281-2285; Ames, B. N. et al., 1973, Proc Natl Acad Sci USA, 70:782-786). PRO206, at the concentration range between 0.5 and 10 μM, was found to be AMES-negative, either in the presence or absence of S9, indicating that PRO206 was not genotoxic.

In summary for PRO206 discovery, a robust HTS-ready HCVpp entry assay was developed; hit finding against a diversified compound library resulted in the discovery of multiple chemotype inhibitors that exhibited potency and selectivity; and hit series were further optimized through multiple iterations of medicinal chemistry.

In summary regarding PRO206's profile, PRO206 exhibited potent activity against a panel of HCVpp; the compound is a highly selective inhibitor of HCV entry; PRO 206's lack of cytotoxicity is indicative of a large therapeutic window; potent antiviral activity of PRO206 demonstrated in the HCV cell culture model; time of addition studies suggest that PRO206 acts post-attachment; and PRO206 exhibits favorable ADME and pharmacokinetic properties in rats.

In accordance with the invention, PRO206 was identified via high throughput screening of a random diversified compound library followed by multiple iterations of chemical optimization. PRO206 is a broadly potent and selective inhibitor of HCV entry, displaying favorable pharmacokinetic properties. Moreover, PRO206 demonstrates potent antiviral activity against authentic HCV in a cell culture efficacy model. The compound exhibits low cytotoxicity against a number of cell lines and displays a favorable ADME, pharmacokinetic and safety profile in preclinical testing. The ability of an antiviral drug to suppress viral replication and prevent the emergence of drug resistant variants is related to its target coverage, defined by the plasma concentration of the drug at trough and the drug's potency (C_{trough}/EC_{K}). After a single oral dose of PRO206, the target coverage of PRO206 at 24 hours was several multiples above the EC₅₀ and is consistent with exploration of once daily dosing in humans. Additionally, the HCV entry inhibitor PRO206 provides drug-like properties and may be suitable for oral administration, alone or in combination with other drugs or agents, such as an all oral combination therapy, for the treatment of HCV infection.

While the invention has been described with respect to preferred embodiments, those skilled in the art will readily appreciate that various changes and/or modifications can be made to the invention without departing from the spirit or scope of the invention as defined by the appended claims.

### REFERENCES CITED

1. World Health Organization (1997) Weekly Epidemiol. Rec. 72, 65.
2. Jenkins, T. M., et al., US 2003/0003448.
3. Lauer, G. M., and B. D. Walker (2001) N. Engl. J. Med., 345, 45.
4. Pawlotsky, J-M. (2005) Semin. Liver Dis., 25, 72.
5. VanCompernolle, S. E. et al. (2003) Virology, 314, 371.
6. Cormier, E. G., et al. (2004) Proc. Nat. Acad. Sci., 101, 7270.
7. Hsu, M., et al. (2003) Proc. Nat. Acad. Sci., 100, 7271.
8. Bartosch, B., et al. (2003) J. Exp. Med., 197, 633.
9. Dumonceaux, J., et al. (2003) J. Virol. 77, 13418.
10. Drummer, H. E., et al. (2003) FEBS Lett., 546, 385.
11. Drummer, H. E., et al. (2002) J. Virol. 76, 11143.
12. Lindenbach, B. D., et al. (2005) Science 309(22), 623
13. Tscherne, D. M. et al. (2006) J. Virol., Vol. 80, 1734
14. Wakita, T. et al. (2005) Nature Medicine, 11, 791
15. Zhong, J. (2005 Proc. Natl. Acad. Sci., 102, 9294
16. Lavillette, D. et al. (2005) Hepatology, 42, 1429
17. Logvinoff, C. et al. (2004) Proc. Natl. Acad. Sci., 101, 10149
18. Meunier, J. C. et al. (2005) Proc. Natl. Acad. Sci., 102, 4560
19. McKeating, J. A. et al. (2004) J. Virol., 78, 8496.
20. Zhang, J., et al. (2004) J. Virol., 78, 1448.
21. Roccasecca, R. et al., (2003) J. Virol., 77(3), 1856.

1. A compound of formula (I), its pharmaceutically acceptable salts, polymorphs, hydrates, stereoisomers, or prodrugs thereof 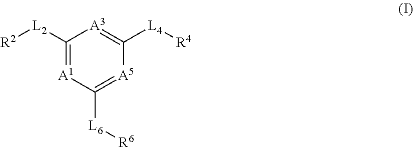
wherein:
A¹, A³, A⁵ are N;
L², L⁴, and L⁶ are independently H, O, S, NR, (CH₂)₀₋₅, CN, CRR′, SO₂, CO, CONR, NHCONR, halide, cycloalkyl, heterocycle, aryl, alkyne, alkene;
R², R⁴, and R⁶ are independently none, R, OR, amino, amine, alkoxy, (CH₂)₀₋₃CF₃, CF₃, (CH₂)₀₋₃W, alkyl, aryl, cycloalkyl, heterocycle, fused alkylaryl or heteroalkylaryl, substituted with 0-2 W;
W is H, halide, OR, CF₃ₕ NO₂, CN, amino, amine, aniline, ester, amide, sulfonamide, sulfone, amino acid, ether, urea acid, heterocycle, alkyl, aryl, arylalkyl, alkylaryl; and
R or R′ are independently H, alkyl, aryl, amide.

2. A compound of formula (Ia), its pharmaceutically acceptable salts, polymorphs, hydrates, stereoisomers, or prodrugs thereof:

4. At least one compound of formula (Ib), its pharmaceutically acceptable salts, polymorphs, hydrates, stereoisomers, or prodrugs thereof 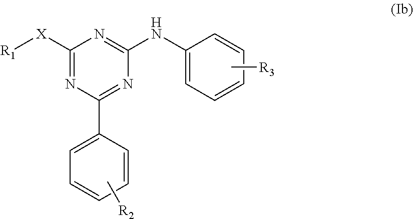
wherein:
X=H, O, NH, CH₂, halide, none;
R₁=H, OH, CF₃(CH₂)ₙ, (un)substituted alkyl or aryl, CN, CF₃;
R₂=halide, CF₃, CN, amide, amine, sulfonamide, (un)substituted alkyl or aryl, hetero ring;
R₃=H, (mono or bis) halide, CF₃, OR, amine, amide, sulfonamide, fused alkyl or aryl ring, hetero ring, (un)substituted alkyl or aryl;
R is independently H, alkyl, aryl, amide; and
n=0-5.

5. A composition comprising at least one of the compounds of claim 1.

6. A composition comprising at least one of the compounds of claim 2.

7. A composition comprising at least one of the compounds of claim 3.

8. A composition comprising at least one of the compounds of claim 4.

9. The composition of any of claims 5-8 and a pharmaceutically acceptable carrier.

10. A formulation of the composition of claim 9 selected from the group consisting of; a solid formulation, a semisolid formulation, a solution formulation, an aqueous formulation, an immediate release formulation, a sustained release formulation, an enteric coating formulation and a lyophilized formulation.

11. The formulation of claim 10, wherein the formulation is a packaged unit dosage.

12. The formulation of claim 11, wherein the packaged unit dosage is a solution, solid, powder, aerosol, liquid or gel.

13. The composition of claim 9, comprising at least one additional antiviral agent.

14. The composition of claim 13, wherein the at least one additional antiviral agent is selected from anti-Hepatitis C virus compounds, anti-HCV Antibodies, Hepatitis C virus protease inhibitors, Hepatitis C virus polymerase inhibitors, Hepatitis C virus helicase inhibitors, or a combination thereof.

15. The composition of claim 13, wherein the at least one additional antiviral agent is an interferon-alpha, pegylated interferon-alpha, ribavirin, or a combination thereof.

16. The composition of claim 9, comprising least one pharmaceutical agent that is not an antiviral agent.

17. The composition of claim 16, wherein the at least one pharmaceutical agent that is not an antiviral agent is an anti-infective agent, an anti-cancer agent, or a combination thereof.

18. Use of a composition of claim 9 in the manufacture of a medicament for treating or preventing infection by a virus of the family Flaviviridae, wherein the composition is to be administered to a patient in need thereof in an amount effective to treat or prevent the infection.

19. The use of claim 18, wherein the virus is Hepatitis C virus (HCV).

20. The use of claim 19, wherein HCV is of genotype 1.

21. The use of claim 19, wherein HCV is of genotype 1a, genotype 1b, or a combination thereof.

22. The use of claim 19, wherein HCV is of genotype 2.

23. The use of claim 18, wherein the composition is to be administered by a route selected from oral, parenteral, subcutaneous, intravenous, or a combination thereof.

24. Use of the compound of any of claims 1-4 in the manufacture of a medicament for inhibiting HCV infection of a cell susceptible to HCV infection, wherein the cell is to be contacted with the compound in an amount effective to inhibit HCV infection of the cell.

25. Use of the composition of any of claims 5-8 in the manufacture of a medicament for inhibiting HCV infection of a cell susceptible to HCV infection, wherein the cell is to be contacted with the composition in an amount effective to inhibit HCV infection of the cell.

26. The use of claim 24, wherein the susceptible cell is in a patient and the compound is to be administered to the patient.

27. The use of claim 25, wherein the susceptible cell is in a patient and the composition is to be administered to the patient.

28. The use of claim 26, wherein at least one additional antiviral agent is to be further administered to the patient.

29. The use of claim 28, wherein the at least one additional antiviral agent is selected from the group consisting of anti-Hepatitis C virus compounds, anti-HCV antibodies, Hepatitis C virus protease inhibitors, Hepatitis C virus polymerase inhibitors, Hepatitis C virus helicase inhibitors, or a combination thereof.

30. The use of claim 29, wherein the at least one additional antiviral agent is an interferon-alpha, pegylated interferon-alpha, ribavirin, or a combination thereof.

31. The use of claim 26, wherein at least one pharmaceutical agent that is not an antiviral agent is to be further administered to the patient.

32. The use of claim 31, wherein the at least one pharmaceutical agent that is not an antiviral agent is an anti-infective agent, an anti-cancer agent, or a combination thereof.

33. Use of a compound of any of claims 1-4 in the manufacture of a medicament for preventing or diminishing HCV infection in a subject, wherein the compound is to be administered to the subject in an amount effective to prevent or diminish the HCV infection.

34. The use of claim 33, wherein the compound is to be administered to the subject before, after, or during exposure of the subject to HCV.

35. Use of a composition of any of claims 5-8 in the manufacture of a medicament for preventing or diminishing HCV infection in a subject, wherein the composition is to be administered to the subject in an amount effective to prevent or diminish the HCV infection.

36. The use of claim 35, wherein the composition is to be administered to the subject before, after, or during exposure of the subject to HCV.

37. Use of a compound of any of claims 1-4 in the manufacture of a medicament for reducing exposure of a subject to HCV infection outside or on the external body surface of the subject, wherein the outside or external body surface of the subject is to be contacted with the compound in an amount effective to inactivate or inhibit the virus so as to reduce exposure of the subject to HCV infection.

38. A method of inactivating, inhibiting, decontaminating, or rendering inactive or weakly infective, objects, surfaces, or substances that have been contaminated with HCV, which comprises contacting the objects, surfaces, or substances with a compound of any of claims 1-4, in an amount effective to inactivate, inhibit, decontaminate, or render inactive or weakly infective the HCV.

39. Use of a compound of any of claims 1-4 in the manufacture of a medicament for reducing the occurrence of HCV infection in a population of individuals, wherein the compound is to be administered to the population of individuals in need thereof in an amount effective to reduce the occurrence of HCV infection in the population.

40. A pharmaceutical composition comprising a therapeutically effective amount of a compound of any of claims 1-4 and a pharmaceutically acceptable carrier or excipient.

41. The pharmaceutical composition of claim 40, in combination with at least one additional antiviral active ingredient selected from the group consisting of: interferons, anti-HCV monoclonal antibodies, anti-HCV polyclonal antibodies, HCV RNA polymerase inhibitors, HCV protease inhibitors, IRES inhibitors, helicase inhibitors, antisense compounds, anti-viral small molecules, ribozymes, or a combination thereof.

42. The pharmaceutical composition of claim 41, wherein the at least one antiviral active ingredient is selected from the group consisting of: ribavirin, interferon-α, interferon-α-2β, or a combination thereof.

43. Use of a compound of any of claims 1-4 in the manufacture of a medicament for treating or preventing a liver disease in a subject, wherein the compound is to be administered to the subject in an amount effective to inhibit infection of the subject's HCV susceptible cells, thereby treating or preventing the liver disease in the subject.

44. The use of claim 43, wherein at least one additional antiviral agent selected from the group consisting of: anti-Hepatitis C virus compounds, anti-HCV antibodies, Hepatitis C virus protease inhibitors, Hepatitis C virus polymerase inhibitors, Hepatitis C virus helicase inhibitors, or a combination thereof, is to be further administered to the subject.

45. The use of claim 44, wherein the at least one additional antiviral agent is an interferon-alpha, pegylated interferon-alpha, ribavirin, or a combination thereof.

46. The use of claim 43, wherein at least one pharmaceutical agent that is not an antiviral agent is to be further administered to the subject.

47. The use of claim 46, wherein the at least one pharmaceutical agent that is not an antiviral agent is an anti-infective agent, an anti-cancer agent, or a combination thereof.

48. Use of a composition of any of claims 5-8 in the manufacture of a medicament for treating or preventing a liver disease in a subject, wherein the composition is to be administered to the subject in an amount effective to inhibit infection of the subject's HCV susceptible cells, thereby treating or preventing the liver disease in the subject.

49. The use of claim 48, wherein at least one additional antiviral agent selected from the group consisting of: anti-Hepatitis C virus compounds, anti HCV antibodies, Hepatitis C virus protease inhibitors, Hepatitis C virus polymerase inhibitors, Hepatitis C virus helicase inhibitors, or a combination thereof, is to be further administered to the subject.

50. The use of claim 49, wherein the at least one additional antiviral agent is an interferon-alpha, pegylated interferon-alpha, ribavirin, or a combination thereof.

51. The use of claim 49, wherein at least one pharmaceutical agent that is not an antiviral agent is to be further administered to the subject.

52. The use of claim 51, wherein the at least one pharmaceutical agent that is not an antiviral agent is an anti-infective agent, an anti-cancer agent, or a combination thereof.

53. Use of a compound of any of claims 1-4 in the manufacture of a medicament for treating or preventing an HCV associated disorder in a subject, wherein the compound is to be administered to the subject in an amount effective to inhibit infection of the subject's HCV susceptible cells, thereby treating or preventing the HCV associated disorder in the subject.

54. Use of a composition of any of claims 5-8 in the manufacture of a medicament for treating or preventing an HCV associated disorder in a subject, wherein the composition is to be administered to the subject in an amount effective to inhibit infection of the subject's HCV susceptible cells, thereby treating or preventing the HCV associated disorder in the subject.

55. Use of a compound of any of claims 1-4, or a composition containing the compound, in the manufacture of a medicament for reducing or preventing HCV infection or recurrence in a liver transplant patient, wherein the compound or composition is to be administered to the patient in an amount effective to reduce or prevent HCV infection or recurrence in the liver transplant patient.

56. The use of claim 55, wherein the compound is to be administered to the patient at a time selected from prior to, at the time of, or following the liver transplant, or a combination thereof.

57. The use of claim 55, wherein the compound is to be administered to the patient prior to the liver transplant.

58. The use of claim 55, wherein the compound is to be administered to the patient at the time of the liver transplant.

59. The use of claim 55, wherein the compound is to be administered to the patient following the liver transplant.

60. The use of claim 55, wherein the compound is to be administered to the patient prior to, at the time of and following the liver transplant.

61. The use of claim 55, wherein the compound is to be administered to the patient in combination with at least one other antiviral drug or therapeutic.

62. The use of claim 61, wherein the at least one other antiviral drug or therapeutic is selected from the group consisting of: anti-HCV compounds, anti-HCV antibodies, HCV protease inhibitors, HCV polymerase inhibitors, HCV helicase inhibitors, or a combination thereof.

63. The use of claim 62, wherein the at least one other antiviral drug or therapeutic is an interferon-alpha, pegylated interferon-alpha, ribavirin, or a combination thereof.

64. The use of claim 55, wherein the compound is to be administered to the patient in combination with at least one other pharmaceutical agent that is not an antiviral agent.

65. The use of claim 64, wherein the at least one pharmaceutical agent is an anti-infective agent, an anti-cancer agent, or a combination thereof.

66. The formulation of claim 10 wherein the formulation is an oral formulation.

67. A compound of formula (Ic), its pharmaceutically acceptable salts, polymorphs, hydrates, stereoisomers, or prodrugs thereof 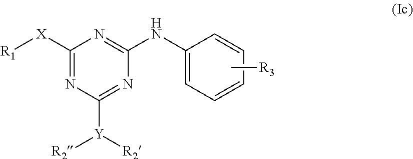
wherein:
X=H, O, NH, CH₂, halide, none;
Y=N, O(R₂″ is none), CH, alkene, alkyne;
R₁=H, OH, CF₃(CH₂)ₙ, (un)substituted alkyl or aryl, CN, CF₃;
R₂′, R₂″, independently, is H, (un)substituted alkyl, aryl, (CH₂)ₙR, (CH₂)ₙAr, (CH₂)ₙSO₂NRR, or together form a (un)substituted hetero ring;
R₃=H, (mono or bis) halide, CF₃, OR, amine, amide, sulfonamide, fused alkyl or aryl ring, hetero ring, (un)substituted alkyl or aryl;
R is independently H, alkyl, aryl, amide; and
n=0-5.

68. A compound of formula (Id), its pharmaceutically acceptable salts, polymorphs, hydrates, stereoisomers, or prodrugs thereof 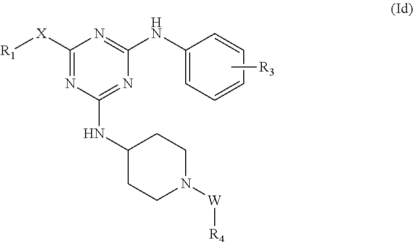
wherein:
X=H, O, NH, CH₂, halide, none;
R₁=H, OH, CF₃(CH₂)ₙ, (un)substituted alkyl or aryl, CN, CF₃;
R₃=H, (mono or bis) halide, CF₃, OR, amine, amide, sulfonamide, fused alkyl or aryl ring, hetero ring, (un)substituted alkyl or aryl;
R₄=(un)substituted alkyl, aryl, amine;
W=SO₂, CO, CH₂, none;
R is independently H, alkyl, aryl, amide; and
n=0-5.

69. A compound of formula (Ie), its pharmaceutically acceptable salts, polymorphs, hydrates, stereoisomers, or prodrugs thereof 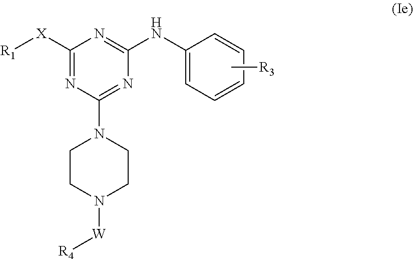
wherein:
X=H, O, NH, CH₂, halide, none;
R₁=H, OH, CF₃(CH₂)ₙ, (un)substituted alkyl or aryl, CN, CF₃;
R₃=H, (mono or bis) halide, CF₃, OR, amine, amide, sulfonamide, fused alkyl or aryl ring, hetero ring, (un)substituted alkyl or aryl;
R4=(un)substituted alkyl, aryl, amine;
W=SO₂, CO, CH₂, none;
R is independently H, alkyl, aryl, amide; and
n=0-5.

70. A compound of formula (If), its pharmaceutically acceptable salts, polymorphs, hydrates, stereoisomers, or prodrugs thereof 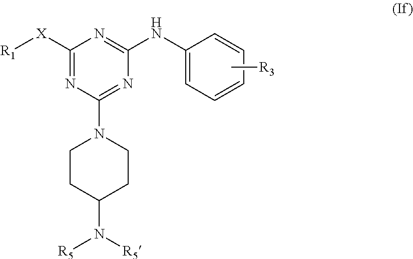
wherein:
X=H, O, NH, CH₂, halide, none;
R₁=H, OH, CF₃(CH₂)ₙ, (un)substituted alkyl or aryl, CN, CF₃;
R₃=H, (mono or bis) halide, CF₃, OR, amine, amide, sulfonamide, fused alkyl or aryl ring, hetero ring, (un)substituted alkyl or aryl;
R₅, R₅′, independently, is (un)substituted alkyl, aryl, sulfonamide, amide;
R is independently H, alkyl, aryl, amide; and
n=0-5.
